(19) United States
(12) Patent Application Publication
(10) Pub. No.: US 2007/0100561 A1
(43) Pub. Date: May 3, 2007

(54) Crystals of DPP-IV

(76) Inventors: Michael Hennig, Weil am Rhein (DE); Bernd Michael Loeffler, Oberrimsingen (DE); Ralf Thoma, Grenzach-Wyhlen (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/635,943

(22) Filed: Dec. 8, 2006

Related U.S. Application Data
(63) Continuation of application No. 10/722,049, filed on Nov. 25, 2003, now abandoned.

(30) Foreign Application Priority Data
Nov. 25, 2002 (EP) 02026367.9

(51) Int. Cl. G06F 19/00; C12N 9/64

(52) U.S. Cl. 702/19; 435/226

(57) ABSTRACT

The present invention provides a crystal of the extracellular domain of mammalian DPP-IV, wherein the crystal has an orthorhombic space group of P2₁2₁2₁ and one homodimer of DPP-IV in the asymmetric unit. Also provided is a co-crystal which includes a ligand bound to the active site of mammalian DPP-IV. The invention permits the identification or design of inhibitor compounds of DPP-IV activity, for use in treatment of type II diabetes.

### PRIORITY TO RELATED APPLICATIONS

This application is a continuation of U.S. application Ser. No. 10/722,049, filed Nov. 25, 2003, now pending; which claims the benefit of European Application No. 02026367.9, filed Nov. 25, 2002. The entire contents of the above-identified applications are hereby incorporated by reference.

### BACKGROUND OF THE INVENTION

Dipeptidyl peptidase (DPP-IV; T-cell activation antigen CD26 or adenosine binding protein) is a multifunctional type II cell surface glycoprotein. The protein is widely expressed in a variety of cell types, particularly on differential epithelial cells of the intestine, liver, prostate tissue, corpus luteum, and kidney proximal tubules (Hartel, S., Gossrau, R., Hanski, C. & Reutter, W. (1988). Dipeptidyl peptidase (DPP) IV in rat organs. Comparison of immunohistochemistry and activity histochemistry. Histochemistry 89, 151-161; McCaughan, G. W., Wickson, J. E., Creswick, P. F. & Gorrell, M. D. (1990). Identification of the bile canalicular cell surface molecule GP110 as the ectopeptidase dipeptidyl peptidase IV: an analysis by tissue distribution, purification and N-terminal amino acid sequence. Hepatology 11, 534-544) as well as leukocyte subsets (Gorrell, M. D., Wickson, J. & McCaughan, G. W. (1991). Expression of the rat CD26 antigen (dipeptidyl peptidase IV) on subpopulations of rat lymphocytes. Cell. Immunol. 134, 205-215), such as T-helper lymphocytes, and subsets of macrophages (Büihling, F., Kunz, D., Reinhold, D., Ulmer, A. J., Ernst, M., Flad, H. D. & Ansorge, S. (1994). Expression and functional role of dipeptidyl peptidase IV (CD26) on human natural killer cells. Nat. Immun. 13, 270-279) and a soluble form is reported to be present in plasma and urine (Iwaki-Egawa, S., Watanabe, Y., Kikuya, Y. & Fujimoto, Y. (1998). Dipeptidyl peptidase IV from human serum: purification, characterization, and N-terminal amino acid sequence. J. Biochem. 124, 428-433). Human DPP-IV has a short cytoplasmatic tail of six amino acids, a 22 amino acid hydrophobic transmembrane region and a 738 amino acid extracellular domain with ten potential glycosylation sites (Tanaka, T., Camerini, D., Seed, B., Torimoto, Y., Dang, N. H., Kameoka, J., Dahlberg, H. N., Schlossman, S. F. & Morimoto, C. (1992). Cloning and functional expression of the T cell activation antigen CD26. J. Immunol. 149, 481-486).

DPP-IV is involved in many biological processes, including a membrane-anchoring function for the localization of the extracellular enzyme adenosine deaminase (ADA) (Franco, R., Valenzuela, A., Lluis, C. & Blanco, J. (1998). Enzymatic and extraenzymatic role of ecto-adenosine deaminase in lymphocytes. Immunol. Rev. 161, 27-42), participation in cell matrix adhesion by binding to collagen and fibronectin (Loster, K., Zeilinger, K., Schuppan, D. & Reutter, W. (1995). The cysteine-rich region of dipeptidyl peptidase IV (CD 26) is the collagen-binding site. Biochem. Biophys. Res. Commun. 217, 341-348), interaction as a co-receptor for the HIV envelope protein gp 120 (Ohtsuki, T., Tsuda, H. & Morimoto, C. (2000). Good or evil: CD26 and HIV infection. J. Dermatol. Sci. 22, 152-160) and co-stimulatory function during T-cell activation and proliferation (von Bonin, A., Huhn, J. & Fleischer, B. (1998). Dipeptidyl-peptidase IV/CD26 on T cells: analysis of an alternative T-cell activation pathway. Immunol. Rev. 161, 43-53) by interaction with the protein tyrosine phosphatase (CD45) (Torimoto, Y., Dang, N. H., Vivier, E., Tanaka, T., Schlossman, S. F. & Morimoto, C. (1991). Coassociation of CD26 (dipeptidyl peptidase IV) with CD45 on the surface of human T lymphocytes. J. Immunol. 147, 2514-2517).

DPP-IV (EC 3.4.14.5) has postproline dipeptidyl amino peptidase activity, preferentially cleaving X-proline or X-alanine dipeptides from the N-terminus of polypeptides (Hopsu-Havu, V. K. & Glenner, G. G. (1966). A new dipeptide naphthylamidase hydrolyzing glycyl-prolyl-beta-naphthylamide. Histochemie 7, 197-201.) and belongs to the prolyl oligopeptidase family, a group of atypical serine proteases able to hydrolyse the prolyl bond (Cunningham, D. F. & O'Connor, B. (1997). Proline specific peptidases. Biochim. Biophys. Acta 1343, 160-186). It possesses a novel orientation of its catalytic triad residues (Ser-Asp-His) (Ikehara, Y., Ogata, S. & Misumi, Y. (1994). Dipeptidyl-peptidase IV from rat liver. Methods Enzymol. 244, 215-227.), inverse to that found in classical serine proteases (His-Asp-Ser). The cleavage of N-terminal peptides with Pro in the second position is a rate limiting step in the degradation of peptides. The natural substrates of DPP-IV include several chemokines, cytokines, neuropeptides, circulating hormones and bioactive peptides (Lambeir, A. M., Durinx, C., Proost, P., Van Damme, J., Scharpe, S. & De Meester, I. (2001). Kinetic study of the processing by dipeptidyl-peptidase IV/CD26 of neuropeptides involved in pancreatic insulin secretion. FEBS Lett. 507, 327-330.). The wide range of substrates suggests a key regulatory role in the metabolism of peptide hormones and in amino acid transport (Hildebrandt, M., Reutter, W., Arck, P., Rose, M. & Klapp, B. F. (2000). A guardian angel: the involvement of dipeptidyl peptidase IV in psychoneuroendocrine function, nutrition and immune defence. Clin Sci 99, 93-104). Its physiological relevance has been investigated by (Hinke, S. A., Pospisilik, J. A., Demuth, H. U., Mannhart, S., Kuhn-Wache, K., Hoffmann, T., Nishimura, E., Pederson, R. A. & McIntosh, C. H. (2000). Dipeptidyl peptidase IV (DPIV/CD26) degradation of glucagon. Characterization of glucagon degradation products and DPIV-resistant analogs. J. Biol. Chem. 275, 3827-3834).

The finding that DPP-IV is responsible for more than 95% of the degradation of GLP-1 led to an elevated interest in inhibition of this enzyme for the treatment of diabetes type II. Experiments in rats and humans have provided evidence that specific DPP-IV inhibition increased Cₘₐₓ, T_{1/2} and total circulating GLP-1 and decreased plasma glucose. It has been demonstrated that patients with impaired glucose-tolerance (IGT), type-II diabetes and with a secondary failure to respond to sulfonylurea treatment benefit from increased levels of GLP 1 peptides. In addition GLP-1 is effective in type-I diabetic patients due to its glucagono-static effect. More recent investigations show a delay of gastric emptying that could have beneficial effects on satiety and might be relevant for the treatment of obesity. Protection of functional GLP-1 by inhibition of DPP-IV and concomitant activation of the GLP-1 receptor might therefore have a synergistic potential in anti-diabetic drug research (Holst, J. J. & Deacon, C. F. (1998). Inhibition of the activity of dipeptidyl-peptidase IV as a treatment for type 2 diabetes. Diabetes 47, 1663-1670.). Selective and orally available small molecule inhibitors of DPP-IV have been discovered and are now in clinical trials (Villhauer, E. B., Brinkman, J. A., Naderi, G. B., Dunning, B. E., Mangold, B. L., Mone, M. D., Russell, M. E., Weldon, S. C. & Hughes, T. E. (2002). 1-[2-[(5-Cyanopyridin-2-yl)amino]ethylamino]acetyl-2-(S)-pyrrolidinecarbon nitrile: a potent, selective, and orally bioavailable dipeptidyl peptidase IV inhibitor with antihyperglycemic properties. J. Med. Chem. 45, 2362-2365; Pospisilik, J. A., Stafford, S. G., Demuth, H. U., McIntosh, C. H. & Pederson, R. A. (2002). Long-term treatment with dipeptidyl peptidase IV inhibitor improves hepatic and peripheral insulin sensitivity in the VDF zucker rat: a euglycemic-hyperinsulinemic clamp study. Diabetes 51, 2677-2683).

### SUMMARY OF THE INVENTION

The present invention provides a crystal of the extracellular domain of mammalian DPP-IV wherein the crystal has an orthorhombic space group of P2₁2₁2₁ and one homodimer of DPP-IV in the asymmetric unit.

The crystal of the present invention has unit cell dimensions of:

a is from 63 Å to 70 Å;

b is from 66 Å to 70 Å;

c is from 416 Å to 424 Å;

and a P2₁2₁2, symmetry.

Also provided is a co-crystal which includes a ligand bound to the active site of mammalion DPP-IV.

The invention permits the identification or design of inhibitor compounds of DPP-IV activity for use in treatment of type II diabetes.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Sequence alignment of DPP-IV and POP: Amino acid sequence alignment of DPP-IV from human (hDPP-IV) and rat (rDPP-IV, only different residues are shown). The alignment of POP from pork was performed using structural superposition for the α/β-hydrolase domain only, because of a lack of structural homology for the β-propeller domain. The top line gives additional information about the secondary structure of DPP-IV (yellow arrows and red bars), the glycosylation sites with visible electron density (Y), the potential glycosylation sites (marked in red), the disulphide bonds (green lines between cysteins that are involved) and an arrow that indicates the start of the cloned ectodomain. Sequences are highlighted light gray for the transmembrane part, gray for the part of the β-propeller involved in dimerization, green for residues involved in adenosine deaminase binding, blue for the tyrosine that is involved in the stabilization of the oxyanion of the catalytic intermediate and pink for the catalytic residues.

FIG. 2. Overall Structure of DPP-IV: Ribbon diagram of DPP-IV viewed perpendicular to the two-fold axis. The domains are colored dark green and light green for the α/β hydrolase and β-propeller domains of subunit A and dark/light blue for the other subunit, respectively. The overall dimension of the molecule is about 125×80×60 Å³. The active site is highlighted by the catalytic residues in ball and stick representation as well as residues that are identified by mutagenesis data to be important for ADA binding. The proposed location at the cell surface is shown by the schematic drawing of the membrane. This figure was prepared using Molscript (Kraulis, P. J. (1991). MOLSCRIPT: A program to produce both detailed and schematic plots of protein structures. J. Applied Crystallogr. 24, 946-950) and rendered with Raster3D (Merrit, E. A. & Bacon, D. J. (1997). Raster3D: photorealistic molecular graphics. Methods Enzymol. 277, 505-524).

FIG. 3. Ribbon drawing of the β-propeller domains of DPP-IV and POP:

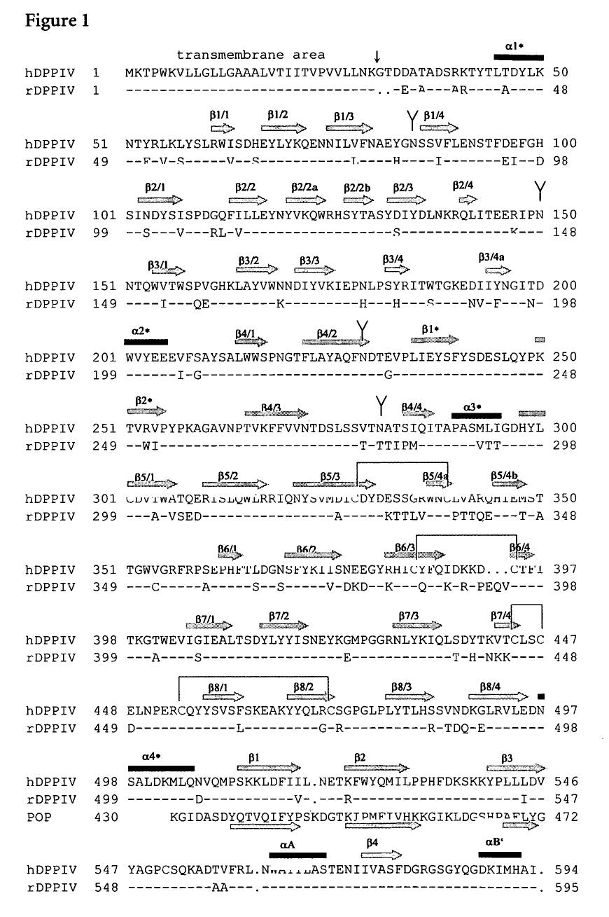

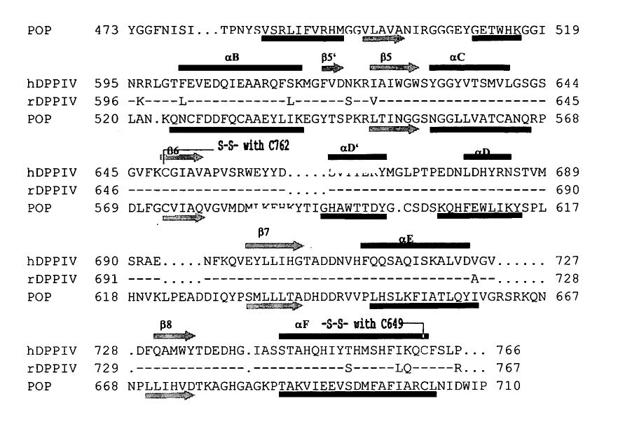

A: DPP-IV has 8 repeats of a structural motif that consists of four antiparallel β-strands or blades (blades are numbered 1 to 8). Additional secondary structural elements are colored magenta: An antiparallel β-sheet (β2/2a and β2/2b in FIG. 1) that is an extension of blade 2 with Arg125 at the tip of the turn that is involved in the substrate binding. An α-helix (α2* in FIG. 1) with the C-terminal glutamate rich loop that contributes to substrate recognition and specificity (Glu204/205/206). The antiparallel β-sheet that forms a main part of the dimer interface (β1* and β2* in FIG. 1). The latter structural elements are extensions of the blade 4.

B: β-propeller domain of DPP-IV rotated 90°

C: POP has 7 blades and no notable deviations from the β-propeller structure. The blades are numbered 1 to 7.

FIG. 4. Access to the active site: Schematic view on the subunit of DPP-IV with the active site surface coloured according to the atom types. The substrate Diprotin A is shown with white carbons indicating the substrate binding site. Arrows illustrate that the substrate may enter the active site at the well accessible and open active site cleft and the dipeptidic product of the catalytic reaction may leave the active site cavity via the more narrow tunnel that is formed by the β-propeller.

FIG. 5. Active site of DPP-IV with Diprotin A (Ile-Pro-Ile): The substrate Diprotin A is trapped as tetrahedral intermediate covalently bound to the active site Ser630. Dashed lines indicate hydrogen bonds. Bonds are dark blue for the protein and light blue for the ligand as well as the active site Ser630. Drawn with MOLOC (Gerber, P. R. (1992). Peptide mechanics: a force field for peptides and proteins working with entire residues as small unites. Biopolymers 32, 1003-1017). The insert shows the omit electron density (ligand and Ser630 were omitted from the calculations) contoured at 2.5 σ (green) and 4 σ (yellow).

### DETAILED DESCRIPTION OF THE INVENTION

The present invention relates to crystals of mammalian DPP-IV, with or without a ligand bound in the active site, where the crystals are of sufficient quality and size to allow for the determination of the three-dimensional X-ray diffraction at atomic resolution. The invention also relates to methods for producing and crystallizing the mammalian DPP-IV. The crystals of mammalian DPP-IV, as well as information derived from their crystal structures can be used to analyze and modify mammalian DPP-IV activity as well as to identify compounds that interact with DPP-IV.

In one aspect the present invention provides a crystal of the extracellular domain of mammalian DPP-IV, preferably having the orthorhombic space group symmetry P2₁2₁2₁ and one homodimer of DPP-IV in the asymmetric unit. Preferably, the crystal includes a unit cell having dimensions a, b, and c; wherein a is from 63 Å to 67 Å, b is from 66 Å to 70 Å, and c is from 416 Å to 424 Å; and α=β=γ=90°. Preferably, the crystal includes atoms arranged in a spatial relationship represented by the atomic structure coordinates listed in Table 4. Preferably, the crystal includes DPP-IV comprising the amino acid sequence from Gly31 to Pro766 of the native protein as well as shorter variants thereof comprising all amino acids necessary for forming the active site. Preferably, the crystal includes DPP-IV as set forth in SEQ ID NO:2 as well as shorter variants thereof comprising all amino acids necessary for forming the active site.

The crystals of the invention include apo crystals and co-crystals. The apo crystals of the invention refer to crystals of mammalian DPP-IV formed without a bound active site or allosteric ligand. The co-crystals generally comprise DPP-IV with a ligand bound to the active site or to an allosteric site. The “active site” refers in general to the site where the enzymatic reaction catalyzed by the enzyme takes place. An active site ligand refers to any compound which specifically binds to the active site of a mammalian DPP-IV.

Preferably, the co-crystal of the present invention is characterized as having an orthorhombic space group of P2₁2₁2₁ (space group No. 19) and one homodimer of DPP-IV in the asymmetric unit.

More preferably, the co-crystal has unit cell dimensions of a is from 63 Å to 67 Å, b is from 66 Å to 70 Å, and c is from 416 Å to 424 Å.; and α=β=γ=90° and a P2₁2₁2₁ symmetry.

The co-crystals of the invention generally comprise a crystalline DPP-IV polypeptide in association with one or more compounds at an active or allosteric binding site of the polypeptide. The association may be covalent or non-covalent.

The DPP-IV (dipeptidyl-peptidase, DPP-IV; T-cell activation antigen CD26 or adenosine binding protein) of the present invention may be a mammalian DPP-IV. Preferably, the DPP-IV of the present invention is a human DPP-IV. More preferably, the DPP-IV of the present invention is the extracellular domain of DPP-IV. Even more preferred is the extracellular domain of DPP-IV which is soluble. Most preferably, the human DPP-IV comprises the amino acid sequence from Gly31 to Pro766 of the native protein as well as shorter variants thereof comprising all amino acids necessary for forming the active site. Preferably, DPP-IV comprises the amino acid sequence as set forth in SEQ. ID NO:2 as well as shorter variants thereof comprising all amino acids necessary for forming the active site.

It is to be understood that the crystals of DPP-IV of the invention are not limited to naturally occurring or native DPP-IV. Indeed, the crystals of the invention include mutants of the native DPP-IV. Mutants of native DPP-IV are obtained by replacing at least one amino acid residue in a native DPP-IV domain with a different amino acid residue, or by adding or deleting amino acid residues within the native polypeptide or at the N- or C-terminus of the native polypeptide, and have substantially the same three-dimensional structure as the native DPP-IV from which the mutant is derived.

By having substantially the same three-dimensional structure is meant having a set of atomic structure coordinates from an apo- or co-crystal that have a root mean square deviation of less than or equal to about 1.5 Å when superimposed with the atomic structure coordinates of the native DPP-IV when at least 50% of the alpha carbon atoms of DPP-IV are included in the superposition.

In some instances, it may be particularly advantageous or convenient to substitute, delete and/or add amino acid residues to a native DPP-IV domain in order to provide convenient cloning sites in cDKA encoding the polypeptide, to aid in purification of the polypeptide, etc. Such substitutions, deletions and/or additions which do not substantially alter the three dimensional structure of the native DPP-IV will be apparent to those having skills in the art.

It should be noted that the mutants contemplated herein need not exhibit DPP-IV activity. Indeed, amino acid substitutions, additions or deletions that interfere with the peptidase activity of the DPP-IV but which do not significantly alter the three-dimensional structure of the domain are specifically contemplated by the invention. Such crystalline polypeptides, or the atomic structure coordinates obtained therefrom, can be used to identify compounds that bind to the native domain. These compounds may affect the activity or the native domain.

The derivative crystals of the invention generally comprise a crystalline DPP-IV polypeptide in covalent association with one or more heavy metal atoms. The polypeptide may correspond to a native or a mutated DPP-IV. Heavy metal atoms useful for providing derivative crystals include, by way of example and not limitation, gold and mercury. Alternatively, derivative crystals can be formed from proteins which have heavy atoms incorporated into one or more amino acids, such as seleno-methionine substitutions for methionine.

Therefore, in a preferred embodiment of the present invention the co-crystal is a co-crystal of the extracellular domain of mammalian DPP-IV and HgCl₂.

The native and mutated DPP-IV polypeptides described herein may be isolated from natural sources or produced by methods well known to those skilled in the art of molecular biology. Expression vectors to be used may contain a native or mutated DPP-IV polypeptide coding sequence and appropriate transcriptional and/or translational control signals. These methods include in vitro recombinant DNA techniques, synthetic techniques and in vivo recombination/genetic recombination. See, for example, the techniques described in Maniatis et al., 1989, Molecular Cloning: A Laboratory Manual, Cold Spring Harbor Laboratory, NY; and Ausubel et al., 1989, Current Protocols in Molecular Biology, Greene Publishing Associates and Wiley Interscience, NY.

A variety of host-expression vector systems may be utilized to express the DPP-IV coding sequence. These include but are not limited to microorganisms such as bacteria transformed with recombinant bacteriophage DNA, plasmid DNA or cosmid DNA expression vectors containing the DPP-IV coding sequence; yeast transformed with recombinant yeast expression vectors containing the DPP-IV coding sequence; insect cell systems infected with recombinant virus expression vectors (e.g. baculovirus) containing the DPP-IV coding sequence; plant cell systems infected with recombinant virus expression vectors (e.g., cauliflower mosaic virus, CaMV; tobacco mosiac virus, TMV) or transformed with recombinant plasmid expression vectors (e.g., Ti plasmid) containing the DPP-IV coding sequence; or animal cell systems. The expression elements of these systems vary in their strength and specificities. Depending on the host/vector system utilized, any of a number of suitable transcription and translation elements, including constitutive and inducible promoters such as pL of bacteriophage μ, plac, ptrp, ptac (ptrp-lac hybrid promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35 S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of the DPP-IV coding sequence, SV40-, BPV- and EBV-based vectors may be used with an appropriate selectable marker.

In a preferred embodiment of the present invention, an isolated nucleic acid sequence encoding the soluble extracellular domain of DPP-IV comprising the nucleotide sequence of SEQ ID NO:1 is provided.

Additionally, an expression vector containing an isolated nucleic acid sequence encoding the soluble extracellular domain of DPP-IV comprising the nucleotide sequence of SEQ ID NO:1 is provided. Preferably, the expression vector for the expression of proteins in P. pastoris which are to be secreted. Furthermore, a host cell transformed with the said expression vector is provided. Preferably, the host cell is Pichia pastoris.

A further aspect of the present invention relates to a method of producing the soluble extracellular domain of DPP-IV comprising culturing the host cell with the said expression vector under conditions permitting the expression of the soluble extracellular domain of DPP-IV by the host cell. Preferably, the host cell is P. pastoris. The present invention also provides the soluble extracellular domain of DPP-IV produced by this method.

Furthermore, the present invention relates to a polypeptide comprising the soluble extracellular domain of DPP-IV as set forth in SEQ ID NO:2.

The apo-, derivative and co-crystals of the invention can be obtained by techniques well-known in the art of protein crystallography, including batch, liquid bridge, dialysis, vapor diffusion and hanging drop methods (see e.g. McPherson, 1982, Preparation and Analysis of Protein Crystals, John Wiley, NY; McPherson, 1990, Eur. J. Biochem. 189:1-23; Webber, 1991, Adv. Protein Chem. 41:1-36; Crystallization of Nucleic Acids and Proteins, Edited by Arnaud Ducruix and Richard Giege, Oxford University Press; Protein Crystallization Techniques, Strategies, and Tips, Edited by Terese Bergfors, International University Line, 1999). Generally, the apo- or co-crystals of the invention are grown by placing a substantially pure DPP-IV polypeptide in an aqueous buffer containing a precipitant at a concentration just below that necessary to precipitate the protein. Water is then removed from the solution by controlled evaporation to produce crystallizing conditions, which are maintained until crystal growth ceases.

Preferably, the crystals are produced by a method for crystallizing mammalian DPP-IV, the method comprising (a) providing a buffered, aqueous solution of pH 7 to 8.5 with a concentration of 7 mg/ml to 22 mg/ml of the extracellular domain of mammalian DPP-IV; and (b) growing crystals by vapor diffusion using a buffered reservoir solution with between 10% and 30% PEG, between 10% and 20% glycerol, wherein PEG has an average molecular weight between 1000 and 20000. More preferably, the extracellular domain of mammalian DPP-IV of step (a) of the method is produced in the yeast Pichia pastoris (P. pastoris) and then deglycosylated. For deglycosylation, different enzymes may be used comprising Endoglycosidase F or PNGase.

Preferably, co-crystals are produced by a method for co-crystallizing mammalian DPP-IV and an active site ligand, the method comprising (a) providing a buffered, aqueous solution of pH 7 to 8.5 with a concentration of 7 mg/ml to 22 mg/ml of the extracellular domain of mammalian DPP-IV; (b) adding a molar excess of the active site ligand to the aqueous solution of mammalian DPP-IV; (c) growing crystals by vapor diffusion using a buffered reservoir solution with between 10% and 30% PEG, between 10% and 20% glycerol, wherein PEG has an average molecular weight between 1000 and 20000. More preferably, the extracellular domain of mammalian DPP-IV of step (a) of the method is produced in P. pastoris and then deglycosylated.

A further aspect of the present invention relates to a crystal produced by the methods for crystallizing or co-crystallizing DPP-IV of the present invention.

Crystals may be frozen prior to data collection.

The mosaic spread of the frozen crystals could sometimes be reduced by annealing, wherein the stream of cold nitrogen gas is briefly blocked, allowing the frozen crystal to thaw momentarily before re-freezing in the nitrogen gas stream.

Diffraction data typically extending to 2.7 Å was collected from the frozen crystals at the synchrotron beamline x06 at the Swiss light source (SLS), Villigen Switzerland. Under optimum conditions, data extending to 2.1 Å was recorded. Preferably, the data is collected at a resolution of 3.5 Å to 2.1 Å or better. More preferably, the data is collected at a resolution of 2.7 Å to 2.1 Å or better.

Derivative crystals of the invention can be obtained by soaking apo or co-crystals in mother liquor containing salts of heavy metal atoms, according to procedures known to those of skill in the art of X-ray crystallography.

Co-crystals of the invention can be obtained by soaking an apo crystal in mother liquor containing a ligand that binds to the active site, or can be obtained by co-crystallizing the DPP-IV polypeptide in the presence of one or more ligands that bind to the active site or to an allosteric site. Preferably, co-crystals are formed with an active site DPP-IV ligand which is slowly hydrolysable and forms a covalent bond. One example for such an active site ligand is Diprotin A.

In a further embodiment of the present invention a method for determining the three-dimensional structure of a crystallized extracellular domain of mammalian DPP-IV to a resolution of 3.5 Å to 2.1 Å or better is provided, the method comprising

(a) crystallizing an extracellular domain of mammalian DPP-IV; and

(b) analyzing the extracellular domain of mammalian DPP-IV by X-ray diffraction to determine the three-dimensional structure of the crystallized extracellular domain of mammalian DPP-IV, whereby the three-dimensional structure of a crystallized extracellular domain of mammalian DPP-IV is determined to a resolution of about 3.5 Å to 2.1 Å or better.

The present invention further relates to a machine-readable data storage medium comprising a data storage material encoded with machine readable data which, when using a machine programmed with instructions for using said data, displays a graphical three-dimensional representation of a molecule or molecular complex comprising at least a portion of the extracellular domain of mammalian DPP-IV comprising the amino acids of SEQ ID NO:2, the extracellular domain comprising the ligand binding active site being defined by a set of points having a root mean square deviation of less than about 1.5 Å from points representing the backbone atoms of said amino acids as represented by structure coordinates listed in Table 4.

The crystals of the invention, and particularly the atomic structure coordinates obtained therefrom, have a wide variety of uses. For example, the crystals and structure coordinates described herein are particularly useful for identifying compounds that interact with DPP-IV as an approach towards developing new therapeutic agents. Pharmaceutical compositions of said compounds can be developed, and said compounds can be used for the manufacture of a medicament comprising said compound for the treatment of IGT, type I and type II diabetes, obesity and cancer.

Therefore, the present invention also relates to the use of a crystal or a co-crystal of the invention for the identification and/or design of inhibitors of DPP-IV activity.

Moreover, the present invention relates to a method for identifying a compound that interacts with DPP-IV, comprising the steps of

(a) generating a three-dimensional model of DPP-IV using the structure coordinates listed in Table 4, a root mean square deviation from the backbone atoms of said amino acids of less than 1.5 Å; and

(b) employing said three-dimensional model to design or select a compound that interacts with DPP-IV.

In another aspect, the method further comprises the steps of

(c) obtaining the identified compound; and

(d) contacting the obtained compound with DPP-IV in order to determine the effect the compound has on DPP-IV activity.

The compound in these methods may be a compound that interacts with the active site of DPP-IV or may be a compound that interacts with an allosteric site of DPP-IV. Preferred are compounds which interact with the active site of DPP-IV. Even more preferred are compounds, which show an inhibitory effect on DPP-IV activity in step (d) of the methods of the present invention.

In a further aspect of the present invention the method for identifying a compound that interacts with DPP-IV is a computer-assisted method. Preferably, determining whether the compound is expected to bind to or interfere with the molecule or molecular complex includes performing a fitting operation between the compound and a binding site or substrate binding surface of the molecule or molecular complex, followed by computationally analyzing the results of the fitting operation to quantify the association between, or the interference with, the compound and the binding site. Optionally, the method further includes screening a library of compound. Optionally, the method further includes supplying or synthesizing the compound, then assaying the compound to determine whether it interacts with and has an effect on mammalian DPP-IV activity.

The present invention also relates to the compounds identified by the said methods for identifying a compound that interacts with DPP-IV.

The structure coordinates described herein can be used as phasing models in determining the crystal structures of additional native or mutated DPP-IV, as well as the structures of co-crystals of such DPP-IV with active site inhibitors or activators bound. The structure coordinates, as well as models of the three-dimensional structures obtained therefrom, can also be used to aid the elucidation of solution-based structures of native or mutated DPP-IVs, such as those obtained via NMR. Thus, the crystals and atomic structure coordinates of the invention provide a convenient means for elucidating the structures and functions of DPP-IV or other prolyl oligopeptidases.

For purposes of clarity and discussion, the crystals of the invention will be described by reference to specific DPP-IV exemplary apo crystals and co-crystals. Those skilled in the art will appreciate that the principles described herein are generally applicable to crystals of any mammalian DPP-IV, including, but not limited to DPP-IV.

Increased levels of glucagon like peptide 1 (GLP1) are beneficial for the decrease of plasma glucose in humans. The finding that DPP-IV is responsible for more than 95% of the degradation of GLP-1 led to an elevated interest in inhibition of this enzyme for the treatment of diabetes type II. Experiments in rats and humans have provided evidence that specific DPP-IV inhibition increased Cₘₐₓ, T_{1/2} and total circulating GLP-1 and decreased plasma glucose. It has been demonstrated that patients with impaired glucose-tolerance (IGT), type-II diabetes and with a secondary failure to respond to sulfonylurea treatment benefit from increased levels of GLP1 peptides. In addition GLP-1 is effective in type-I diabetic patients due to its glucagono-static effect. More recent investigations show a delay of gastric emptying that could have beneficial effects on satiety and might be relevant for the treatment of obesity. Protection of functional GLP-1 by inhibition of DPP-IV and concomitant activation of the GLP-1 receptor might therefore have a synergistic potential in anti-diabetic drug research (Holst, J. J. & Deacon, C. F. (1998). Inhibition of the activity of dipeptidyl-peptidase IV as a treatment for type 2 diabetes. Diabetes 47, 1663-1670). Selective and orally available small molecule inhibitors of DPP-IV have been discovered and are now in clinical trials.

Therefore, in a further aspect of the present invention a pharmaceutical composition comprising the compound identified by the methods of the present invention as having an effect on DPP-IV activity, or pharmaceutically acceptable salts thereof, and a pharmaceutically acceptable carrier is provided.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, benzenesulfonic, toluenesulfonic, methanesulfonic, ethane disulfonic, oxalic, isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

“Stable compound” and “stable structure” are meant to indicate a compound that is sufficiently robust to survive isolation to a useful degree of purity from a reaction mixture, and formulation into an efficacious therapeutic agent.

Furthermore, a compound identified by the methods of the present invention as having an effect on DPP-IV activity for use as a therapeutic active substance, in particular for the treatment of diabetes type I, diabetes type II, IGT, obesity and cancer, is provided.

A further aspect of the present invention relates to the use of a compound identified by the methods of the present invention as having an effect on DPP-IV activity for the manufacture of a medicament for the treatment of diabetes type-I, diabetes type-II, IG, obesity, and cancer.

Having now generally described this invention, the same will become better understood by reference to the specific examples, which are included herein for purpose of illustration only and are not intended to be limiting unless otherwise specified, in connection with the following figures.

### EXAMPLES

Commercially available reagents referred to in the examples were used according to manufacturer's instructions unless otherwise indicated.

### Example 1

### DNA Manipulation and Sequence Analysis

Preparation of DNA probes, digestion with restriction endonucleases, DNA ligation and transformation of E. coli strains were performed as described (Sambrook, J., Fritsch, E. F. & Maniatis, T. (1989). Molecular Cloning: A Laboratory Manual. Cold Spring Harbor Laboratory Press: Cold Spring Harbor, N.Y.). For DNA sequencing, the ABI PRISM BigDye Terminator Cycle Sequencing Ready Reaction Kit and ABI PRISM 310 Genetic analyzer were used. PCR were performed in the T3 Thermocycler (Whatman Biometra), using the Pfu polymerase (Stratagene).

Production and Purification of Recombinant Human sDPP-IV in P. pastoris

The ectodomain of DPP-IV, residues 31-766 (sDPP-IV), was amplified by PCR using a cDNA and the oligonudeotides 5′-TGCTGGAATTCGGCACAGATGATGCTAC-3′ (with an EcoRI site in bold) and 5′-GCA TGG TAC CTT GAG GTG CTA AG-3′ (with a KpnI site in bold). Using the two new restriction sites, the amplified DNA fragment (SEQ ID NO:1) was cloned into pPICZα-A vector (Invitrogen) to create a fusion with the α-mating factor signal sequence for the secretion of the protein. The use of the EcoRI restriction site added the amino acids glutamine and phenylalanine to the N-terminus of sDPP-IV. The sequence was confirmed by sequencing. pPICZα-sDPP-IV was linearized with SacI, transformed by electroporation in P. pastoris strain GS115 and the phenotype of the colonies obtained was checked as recommended by the distributor Invitrogen.

Eight transformants with phenotype MutS were screened for the expression of DPP-IV. Colonies were grown at 30° C. in YPD medium (1% yeast extract, 2% peptone, 2% glucose) with zeocin (100 μg/ml) to an OD₆₀₀ of 8-10. Cells were collected by centrifugation and resuspended in YP medium plus 2% methanol. The same amount of methanol was added every 24 h. After 48 h the medium of each clone was tested for activity (see below). sDPP-IV was then produced in a large scale culture using the transformed cell line with the highest activity per volume as described (Dale, G. E., D'Arcy, B., Yuvaniyama, C., Wipf, B., Oefner, C. & D'Arcy, A. (2000). Purification and crystallization of the extracellular domain of human neutral endopeptidase (neprilysin) expressed in Pichia pastoris. Acta Crystallogr. D 56, 894-897).

Ten liters of the collected sDPP-IV supernatant of the selected transformed P. pastoris cell line was filtered and concentrated to 180 ml by crossflow ultrafiltration (skannette) using a 30 kDA filtration module (AGT Technology corporation). The concentrate was passed over a Sephacryl 200 XK 50/100 size exclusion column (5×95 cm, Pharmacia) equilibrated with 50 mM Tris-HCl pH 7.8 and 100 mM NaCl (S-buffer). Collected fractions were screened on SDS-PAGE and for activity. Fractions containing sDPP-IV were dialysed against 50 mM Tris-HCl pH 7.9. The protein solution was loaded on a Fractogel-TMAE column (2.6×13 cm, Merck) equilibrated with 50 mM Tris-HCl pH 7.9, washed with two column volumes of the same buffer and eluted with 500 ml of a linear gradient from 0 to 200 mM NaCl. Fractions containing sDPP-IV were dialysed against 20 mM sodium acetat pH 4.8. The protein solution was loaded on a Fractogel-COO⁻ column (1×12 cm, Merck) equilibrated with the same buffer and washed with two column volumes of this buffer. Bound proteins were eluted with 200 ml of a linear gradient from 50 to 500 M NaCl. The elution profile showed a major peak at 250 mM NaCl. Preparation of enzymatically deglycosylated sDPP-IV (sDPPIV_{deglycos}) was carried out prior to loading on the last gelfiltration column. 0.1% EndoF1-GST was added to the pooled fractions of DPP-IV and incubated for 20 h at 21° C. The concentrated protein solution was loaded on a Biosec size exclusion column (1.6×60 cm, Merck), that was equilibrated with S-buffer. Fractions were analyzed by SDS-PAGE, showing a purity >95%. N-terminal sequencing showed that the protein was efficiently processed by the STE13 signal peptidase which cleaves off the α-mating factor. Preparation of the sDPPIV_{deglycos}:ADA-complex was performed by addition of a two times excess of ADA (Sigma Type IV, from calf intestinal Mucosa) and purification using a Biosec-size exclusion column.

The soluble extracellular domain of human dipeptidyl peptidase IV (sDPP-IV; residues 31-766) was expressed in the yeast Pichia pastoris. The protein was secreted at the low level of 1 mg/l as estimated from the total activity. As a first purification step the concentrated protein was passed through a size-exclusion column which removed the main fraction of contaminating peptides from the yeast-peptone medium. Sequential chromatography on anion- and cation-exchanger and a second size exclusion chromatography were used to get protein of 95% purity as judged by SDS-PAGE. The yield of pure protein was 0.3 mg/l growth medium. The purified protein shows essentially identical kinetic parameters and inhibition constants for known inhibitors of DPP-IV to those reported for the enzyme purified from human serum (Tables 1 and 2).

Analytical Methods

Purification of sDPP-IV was followed by electrophoresis on 10-20% Tricine SDS polyacrylamide gradient gels (Läimmli, U. K. (1970). Cleavage of structural proteins during assembly of the head of bacteriophage T4. Nature 227, 680-685). Protein concentrations were determined according to Bradford (Bradford, M. M. (1976). A rapid and sensitive method for the quantitation of microgram quantities of protein utilizing the principle of protein-dye binding. Anal. Biochem. 72, 248-254) or for pure protein by absorption spectroscopy using the calculated molecular extinction coefficient at 280 nm of 193'920 M⁻¹cm⁻¹ (A₂₈₀^{0.1%}=2.27 cm²/mg; Pace, C. N., Vajdos, F., Fee, L., Grimsley, G. & Gray, T. (1995). How to measure and predict the molar absorption coefficient of a protein. Protein Sci 4, 2411-2423). Analytical gel filtration chromatography was performed on a Superdex 200 12 HR 10/30 column (Pharmacia) equilibrated with S-buffer. The eluate was monitored with a miniDAWN multi-angle laser light scattering detector (Wyatt) and a refractive index-detector (Shodex), which allows the determination of the molecular weight and dispersity over the elution peak (Wyatt, P. J. (1993). Light scattering and the absolute characterisation of macromolecules. Analytica Chimica Acta 272, 1-40). Sedimentation equilibrium runs in a Beckman analytical ultracentrifuge (model Optima XL A) were performed at 20° C. and 9000 rpm sDPP-IV_{deglycos} and at 7000 rpm for sDPP-IV_{deglycos}:ADA-complex. The initial protein concentrations were 0.22 to 0.25 mg/ml in S-buffer. The absorption was followed at 280 nm. Assumed partial specific volumes for sDPP-IV of 0.729 cm³/g and ADA of 0.735 cm³/g were used to determine the molecular masses.

Free sulfhydryl groups were determined according the procedure described by Ellman (Ellman, G. L. (1959). Tissue sulfhydryl groups. Arch. Biochem. Biophys. 82, 70-77) under denaturing conditions (0.3% SDS in 50 mM Tris pH 8.0).

Thermostability Measurements

The irreversible loss of activity after incubation at various temperatures was used as an operational criterion of the thermostability of sDPP-IV. Kinetics of irreversible heat inactivation were performed as described by Sterner et al. (Sterner, R., Kleemann, G. R., Szadkowski, H., Lustig, A., Hennig, M. & Kirschner, K. (1996). Phosphoribosyl anthranilate isomerase from Thermotoga maritima is an extremely stable and active homodimer. Protein Sci. 5, 2000-2008) with a final protein concentration of 20 μg/ml in 50 mM potassium phosphate buffer at pH 7.5, containing 100 mM NaCl. The residual activity was determined by recording the initial velocity at 25° C. of the enzyme-catalyzed reaction (see below) and the averaged values obtained were plotted against the incubation temperature.

Biacore

DPP-IV was immobilized on a CM5 surface plasmon resonance sensor (Biacore) using standard amide coupling chemistry. The organic adlayer on this sensor type consists of carboxymethylated dextran (MW≈100 kDA). After activation of the carboxylic acid groups using carbodiimide/N-hydroxysuccinimide solutions, the surface was contacted with a DPP-IV solution (80 μl) containing≈100 μg/ml protein in acetate buffer (10 mM, pH 4.5). The amount immobilized corresponded to a sensor response of roughly 10 000 RU. The surfaces of two flow cells were modified with protein. To suppress baseline drift—possibly due to slow dimer dissociation—the protein of one cell was cross-linked by short contact with carbodiimide/N-hydroxysuccinimide solution. This treatment did not influence the protein activity since binding constants determined with cross-linked protein were similar to those determined with non-cross-linked protein. Hepes buffer (0.01 M Hepes, pH 7.4, 0.15 M NaCl, 3 mM EDTA, 0.005% polysorbate 20 (v/v)) was used as the running buffer. Diprotin-A was disolved directly in this buffer. NVP-DPP728 was first dissolved in pure DMSO and then diluted into running buffer. The final inhibitor solution contained less than 0.1% DMSO. Binding experiments were carried out by contacting the immobilized protein surfaces with inhibitor solutions of varying concentrations at a flow rate of 10 μl/min or 30 μl/min. After each contact with inhibitor, the protein surfaces were regenerated by extensively washing with running buffer.

Activity Assay

The activity assay is based on the increase of fluorescence of products compared to the substrate Ala-Pro-7-amido-4-trifluoromethylcoumarin (Calbiochem, Smith, R. E., Reynolds, C. J. & Elder, E. A. (1992). The evolution of proteinase substrates with special reference to dipeptidylpeptidase IV. Histochem. J. 24, 637-647). A 20 mM stock solution in 10% DMF is stored at −20° C. until use. Purification was followed by using a final substrate concentration of 50 μM and for the determination of kinetic parameters it was varied between 1.5 μM and 500 μM in the assay. DPP-IV activity assays were performed in 96 well plates in a total assay volume of 100 μl. The assay buffer consists of S-Buffer containing 0.1 mg/ml BSA. Fluorescence is detected in a Luminescence Spectrometer LS 50B (Perkin Elmer) at an excitation wavelength of 400 nm and an emission wavelength of 505 nm. Initial rate constants are calculated by best fit linear regression.

### Example 2

### Crystallization and Structure Determination

For crystallization trials, sDPP-IV_{deglycos} was concentrated to approximately 10 mg/ml. A reduced factorial screen was carried out using the vapour diffusion method. Crystals were obtained with 20-25% PEG 3350, 200 mM MgCl₂, Tris pH 8.5 and 15% glycerol. The crystals were flash-frozen in liquid nitrogen and exhibit the orthorhombic space group P2₁2₁2₁, with cell dimensions of about 65 Å, 68 Å and 420 Å and one dimer per asymmetric unit. They diffract to a maximum of 2.3 Å resolution using synchrotron radiation and show rather high mosaicity (0.5-1.2°). Addition of 1 mM Diprotin-A prior to crystallization led to crystals of the complex. The mercury derivative was produced by cocrystallization with 0.1 mM HgCl₂.

Data collection was performed using synchrotron radiation (Swiss light source, SLS Villigen, Switzerland and ID14, ESRF Grenoble, France) as well as in-house facilities (search for heavy atom derivatives, evaluation of crystal quality) and processed with DENZO (Otwinowski, Z. (1993). Oscillation data reduction program. In Proceedings of the CCP4 Study Weekend: Data Collection and Processing (Wawyey, L., Isaacs, N. & Bailey, S., eds.). pp. 56-62, SERC Daresbury Laboratory, UK). Details of the data collection statistics are given in Table 3. All programs used are part of the CCP4 (CCP4 (Collaborative Computational Project, Number 4) (1994). The CCP4 suite: programs for protein crystallography. Acta Crystallogr. D, 760-763) suite, except where indicated. The structure was determined by multiwavelength anomalous dispersion (MAD) of the mercury derivative. One major mercury binding site per subunit (Cys 551, one of the two free SH-groups Cys301 and Cys551 that are located near the active site) was identified by inspection of the difference patterson maps calculated from the peak wavelength data and was subsequently refined using SHARP (De la Fortelle, E. & Bricogne, G. (1997). Maximum likelihood heavy-atom parameter refinement for multiple isomorphus replacement and multiwavelength anomalous diffraction methods. Methods Enzymol. 276, 472-494). Location of the twofold non-crystallographic axis was performed using this mercury site and the program find2folds (Dunten, P. & Hennig, M. (2002). Locating non-crystallographic symmetry elements: The program Find2Folds. Acta Crystallogr. A58, C76). Further analysis revealed another site per subunit (Cys301) with less occupancy and the site branched in two positions with about 2.4 Å distance. Subsequently the phases were improved by application of twofold averaging combined with solvent flattening and histogram matching as implemented in DM. The initial electron density at 2.6 Å resolution was readily interpretable and about 90% of the polypeptide chain could be built. The molecular model was refined against 2.3 Å data. Subsequent rounds of manual rebuilding and refinement with REFMAC (Murshudov, G. N., Vagin, A. A., Lebedev, A., Wilson, K. S. & Dodson, E. J. (1999). Efficient anisotropic refinement of macromolecular structures using FFT. Acta Crystallogr. D 55, 247-255) led to a complete molecular structure of the polypeptide chain from residues Ser39 to Pro766. Details of the refined structures are reported in Table 3. Coordinates have been deposited in the Protein Data Bank PDB.

Overall Structure

The structure of human DPP-IV was solved by multiple anomalous dispersion (MAD) using a mercury derivative (see Table 3) and subsequently refined to an R-factor of 21.5% at 2.1 Å resolution. The current model consists of all residues from Ser39 to Pro766 of the amino acid sequence of the expressed ectodomain of the protein.

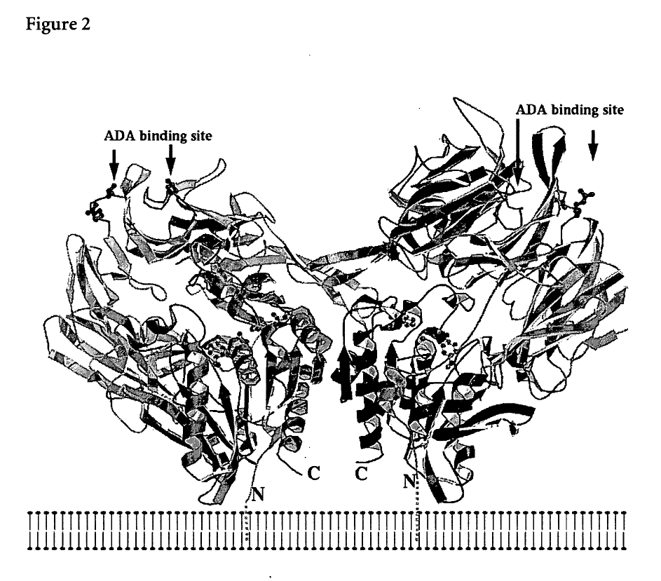

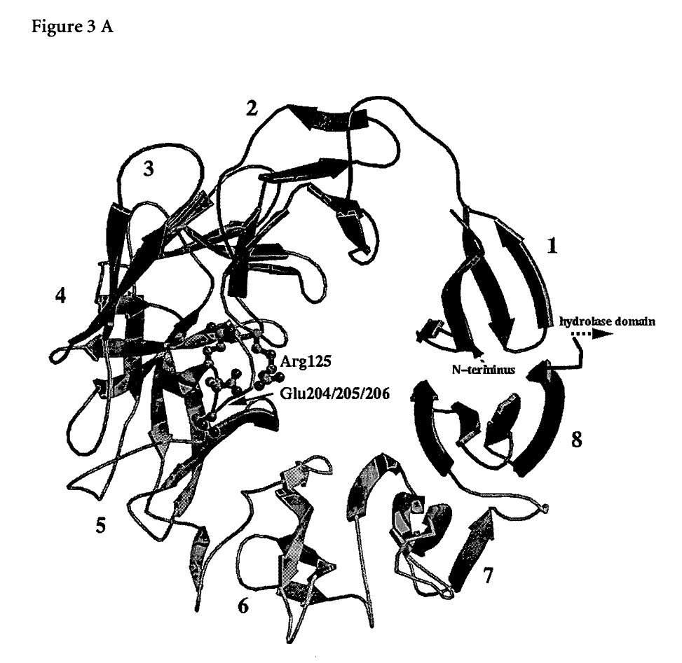

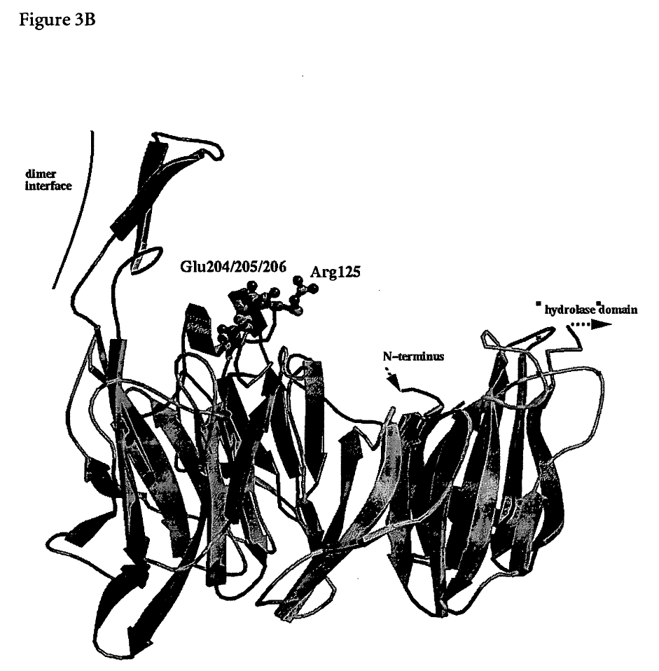

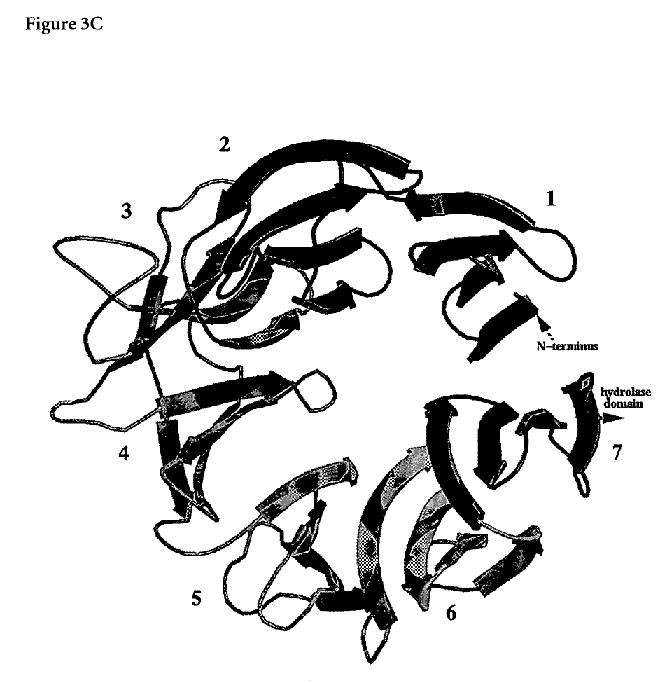

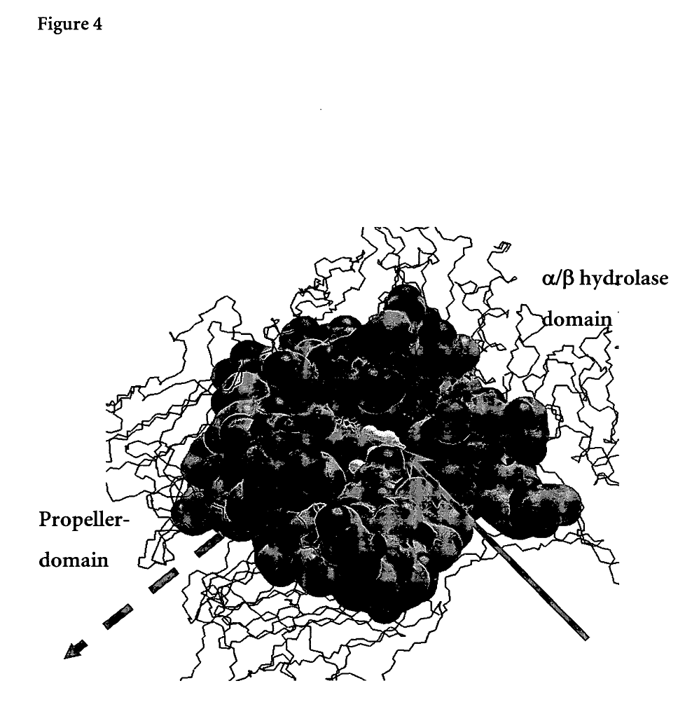

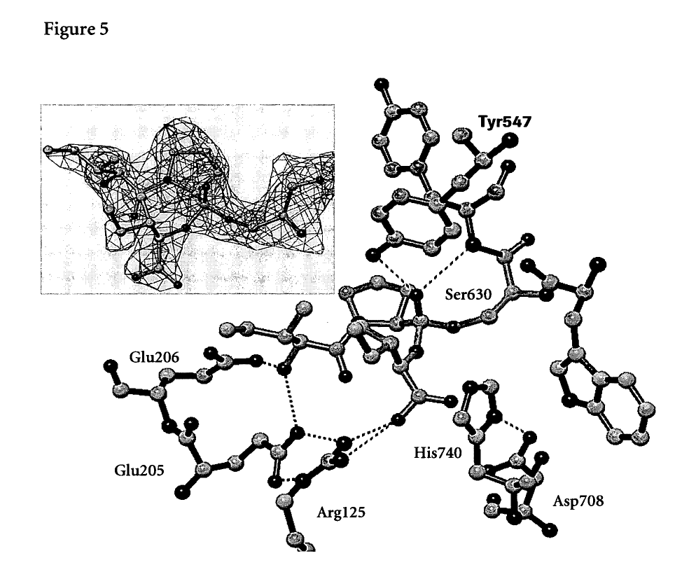

A homodimer of DPP-IV is situated in the asymmetric unit (FIG. 2). Dimerization is also observed in solution under various conditions and is required for activity. Each subunit is made of two domains, the catalytic domain with an α/β hydrolase fold containing the catalytic triad (Ser630, Asp708, His740) and a domain with an eight-bladed β-propeller fold, the β-propeller domain (FIG. 2). The assignment of the secondary structure is given in FIGS. 1 and 2. The only other known crystal structure of this class of enzyme is prolyl-oligopeptidase (POP) determined by Fülop (Fülop, V., Bocskei, Z. & Polgar, L. (1998). Prolyl oligopeptidase: an unusual beta-propeller domain regulates proteolysis. Cell 94, 161-170; pdb entry 1 qfm). POP also has an α/β-hydrolase and a β-propeller domain, but is monomeric and the β-propeller consists of seven repeats only (FIG. 3C).

Catalytic Domain

The catalytic domain is built up of residues Gln508 to Pro766 and contains a central eight-stranded parallel β-sheet that is flanked by 12 helices known as α/β hydrolase fold. 21% sequence identity to POP indicates significant structural homology (FIG. 1) and superposition of the central α-helix, carrying the catalytic Ser630 on its first turn, with the corresponding structure of POP gives an r.m.s deviation of 2.5 Å² for 238 residues. The catalytic domain is connected to the β-propeller by an N-terminal 15 residue linker, which is considerably shorter than the corresponding 76 residue region in POP. The residues lacking in DPP-IV are, however, replaced structurally and functionally by the C-terminal part of the catalytic domain of the second subunit of the dimer.

β-Propeller Domain

The β-propeller domain is formed by the residues Lys56 to Asn497. The preceding N-terminal residues Ser39 to Leu55 form a loop structure with a small α-helix (α*, FIG. 1) at the surface and in close proximity to the first residues of the catalytic domain. The β-propeller domain consists of an eight-fold repeat of a four-stranded antiparallel β-sheet motif (blade, FIG. 3). The blades are in circular arrangement such that they form a solvent filled tunnel with a diameter of about 13 Å.

The β-propeller domain in DPP-IV does not form a joint β-sheet motif (described as molecular “velcro”; Fülop, V. & Jones, D. T. (1999). Beta propellers: structural rigidity and functional diversity. Curr. Opin. Struct. Biol. 9, 715-721; Paoli, M. (2001). Protein folds propelled by diversity. Prog. Biophys. Mol. Biol. 76, 103-130), but rather the blades show a regular arrangement (β1/1 to β7/4 or β8/4) (FIG. 3A) around the central axis forming a ring system that is not closed.

DPP-IV deviates from the regular β-propeller fold by additional secondary structural elements. An anti-parallel β-sheet is inserted in blade two between the strands one and two. The tip of the turn carries the residues Arg125 that forms a salt bridge with Glu205, that is situated at the C-terminal turn of an α-helix (residues Trp154 to Thr199), that is inserted between the first and second strands of blade 4. Arg125, Glu205 and the neighboring Glu204 form a significant part of the substrate binding site and are mainly responsible for the substrate specificity. An further anti-parallel β-sheet motif formed by residues Asp230 to Asn263 is inserted between the strands three and four of blade four (FIG. 3B). This structural element forms a significant part of the dimer interface (see below).

Whereas the N-terminal β-sheet structure of the propeller has shorter strands and is somewhat tilted, the loop connecting the first and second β-sheet is longer, shows high temperature factors and may reduce the rigidity of the propeller architecture. The reduced stability of the circular domain structure at this position might be compensated by an extended hydrophobic cluster that consists of Ile63, Leu69, Ile76, Phe89, Leu90, Phe95, Phe98, Ile107, Ile114, Tyr135, Leu137 and Leu142, and a salt bridge between Arg61 and Asp 104 and a hydrogen bond between the main chain NH of Arg61 and Tyr105. This distortion leads to a reduced height of the propeller at the positions between blade one and two (FIG. 3B).

As no residues from the α/β hydrolase domain fill this up, a cleft between the two domains of the DPP-IV molecule is formed with a diameter of about 15 Å enabling access to the catalytic site (FIG. 4). Therefore, we propose that DPP-IV has two independent ways for the substrate and product to access and leave the active site, a cleft between the domains and the tunnel through the β-propeller. The open cleft may enable large peptides and partially folded proteins to access the active site. The more narrow tunnel could be an exit for the cleaved dipeptides (FIG. 4). The crystal structure of POP shows that the cleft between the two domains does not exist and the tunnel through the β-propeller is more narrow with about 4 Å compared to about 13 Å for DPP-IV (FIGS. 3A and 3C). This structural difference is supported by the observation that DPP-IV can process much larger substrates compared to POP. Peptides with a length of up to about 80 residues appear to be good substrates of DPP-IV. Larger proteins may also be cleaved depending on their tertiary structure. POP is reported to hydrolyse substrates with a maximum size of about 30 residues, only (Polgar, L. (1992). Unusual secondary specificity of prolyl oligopeptidase and the different reactivities of its two forms toward charged substrates. Biochemistry 31, 7729-7735.). As the diameter of the β-propeller tunnel in POP is significantly smaller, it is conceivable that the structure of DPP-IV represents a more open and active enzyme.

The β-propeller motif has been found in several further proteins, but no or only low sequence homology could be demonstrated (Polgar, L. (1992). Unusual secondary specificity of prolyl oligopeptidase and the different reactivities of its two forms toward charged substrates. Biochemistry 31, 7729-7735.). A search of the PDB for homologous structures gave the best results for clathrin (7 blades, ter Haar, E., Musacchio, A., Harrison, S. C. & Kirchhausen, T. (1998). Atomic structure of clathrin: a beta propeller terminal domain joins an alpha zigzag linker. Cell 95, 563-573), methylamine dehydrogenase (7 blades, Chen, L., Doi, M., Durley, R. C., Chistoserdov, A. Y., Lidstrom, M. E., Davidson, V. L. & Mathews, F. S. (1998). Refined crystal structure of methylamine dehydrogenase from Paracoccus denitrificans at 1.75 Å resolution. J. Mol. Biol. 276, 131-149) and nitrite reductase (8 blades, Nurizzo, D., Cutruzzola, F., Arese, M., Bourgeois, D., Brunori, M., Cambillau, C. & Tegoni, M. (1998). Conformational changes occurring upon reduction and NO binding in nitrite reductase from Pseudomonas aeruginosa. Biochemistry 37, 13987-13996), but no DPP-IV related function can be expected.

Active Site

The catalytic triad (Ser630, Asp708, His740) is located in a large cavity at the interface of the two domains. Ser630 is found at the tip of a very sharp turn between β-strand 5 and helix C, called the nucleophile elbow, which is a characteristic of hydrolases of the α/β type (Ollis, D. L., Cheah, E., Cygler, M., Dijkstra, B., Frolow, F., Franken, S. M., Harel, M., Remington, S. J., Silman, I., Schrag, J. & et al. (1992). The alpha/beta hydrolase fold. Protein Eng. 5, 197-211). The serine hydroxy group is well exposed to solvent and hydrogen bonded to the catalytic imidazole group of His740 on one side (2.6 Å) and accessible to the substrate on the other side. His740 is found in the middle of a loop between β-strand 8 and helix F. With a distance of 2.75 Å to Nε of the imidazole ring, one of the oxygen atoms of Asp708 is hydrogen bonded to His740 and completes the catalytic triad (FIG. 5). The other oxygen atom of the carboxylate group of Asp708 is coordinated by two main chain NH-groups (Val711 and Asn710). Thus, the location and geometry of the triad are very similar to that found in other α/β hydrolases with the “handedness” opposite to the classical serine peptidases.

The negatively charged oxyanion of the tetrahedral intermediate is stabilized by the main chain NH-group of Tyr631 and by the hydroxy group of Tyr547 (FIG. 5). Furthermore, the structure shows that the two Gly628 and Gly632 are important for the formation of the sharp turn to bring the catalytic residue Ser630 in the correct position. This is in accordance with mutagenesis studies on rat DPP-IV (Ogata, S., Misumi, Y., Tsuji, E., Takami, N., Oda, K. & Ikehara, Y. (1992). Identification of the active site residues in dipeptidyl peptidase IV by affinity labeling and site-directed mutagenesis. Biochemistry 31, 2582-2587) showing that the sequence Gly₆₂₈-X-Ser₆₃₀-Tyr₆₃₁-Gly₆₃₂ is essential for DPP-IV activity.

Substrate Binding

The substrate binding site of DPP-IV is indicated by the inhibitor Diprotin-A (Ile-Pro-Ile). It is a slowly hydrolysable substrate with k_{cat}/K_{M} a factor of 10 less than Ile-Pro-4-nitroanilides (Rahfeld, J., Schierhorn, M., Hartrodt, B., Neubert, K. & Heins, J. (1991). Are diprotin A (Ile-Pro-Ile) and diprotin B (Val-Pro-Leu) inhibitors or substrates of dipeptidyl peptidase IV? Biochim. Biophys. Acta 1076, 314-316). Inspection of the electron density map shows the ligand covalently bound to the active site Ser630 of the enzyme in both subunits. The N-terminal Ile (P2) and Pro residues (P1) are well defined and enable a detailed analysis of the interaction with the substrate binding site (according to the notation of Schechter; Schechter, I. & Berger, A. (1968). On the active site of proteases. 3. Mapping the active site of papain; specific peptide inhibitors of papain. Biochem. Biophys. Res. Commun. 32, 898-902). Less well defined electron density is found for the C-terminal Ile (P1′), but in subunit B the conformation of this part of the ligand could also be observed (FIG. 5). The side chain NE of the catalytic His740 is in hydrogen bonding distance to the NH-group of P1′ (2.90 Å) and to the Oγ of the Ser630 side chain (2.74 Å).

DPP-IV hydrolyzes oligopeptides and proteins from the N-terminus, cleaving dipeptide units when the second residue is proline, hydroxyproline, dehydroproline, pipecolic acid or alanine. In both subunits the proline in position P1 of Diprotin-A is in the trans-configuration and fits optimally into the pocket of the active site as expected (Fischer, G., Heins, J. & Barth, A. (1983). The conformation around the peptide bond between the P1- and P2-positions is important for catalytic activity of some proline-specific proteases. Biochim. Biophys. Acta 742, 452-462). The S1 pocket is formed by Val711, Val656, Tyr662, Tyr666, Tyr659 and Tyr631 which shape a well defined hydrophobic pocket that would be filled by proline much better than by alanine. Gly is also accepted, but with very low k_{cat}/K_{M} values (Brandt, W., Lehmann, T., Thondorf, I., Born, I., Schutkowski, M., Rahfeld, J. U., Neubert, K. & Barth, A. (1995). A model of the active site of dipeptidyl peptidase IV predicted by comparative molecular field analysis and molecular modelling simulations. Int. J. Pept. Protein Res. 46, 494-507). All other naturally occurring amino acids residues cannot occupy position P1. Either the side chains are too bulky or hydrophilic. The side chains of the residues P2 and P1′ point into the solvent and no interaction with the protein occurs. This explains the large diversity of amino acids accepted in substrates at these positions.

Essential for substrate binding and catalysis is the N-terminus of the substrates, which has to be unprotected and protonated (Brandt, W., Ludwig, O., Thondorf, I. & Barth, A. (1996). A new mechanism in serine proteases catalysis exhibited by dipeptidyl peptidase IV (DP IV)—Results of PM3 semiempirical thermodynamic studies supported by experimental results. Eur. J. Biochem. 236, 109-114). The Diprotin-A complex shows that the terminal —NH₃⁺-group is held very precisely in position by strong interactions with the carboxylates of Glu205 and Glu206 (FIG. 5). A third glutamate, Glu204, stabilizes this substrate recognition site by an hydrogen bonding network with the backbone NH of Arg125, His126 and Ser127 as well as the hydroxy group of Ser127. Importance of the glutamate residues is confirmed by single point mutations that abolish DPP-IV activity (Abbott, C. A., McCaughan, G. W. & Gorrell, M. D. (1999). Two highly conserved glutamic acid residues in the predicted beta propeller domain of dipeptidyl peptidase IV are required for its enzyme activity. FEBS Lett. 458, 278-284). The double Glu-motif is located at the end of an helical segment (α2* in FIG. 1, see also FIG. 3) that is highly conserved in the DPP IV-like gene family (Asp-Trp-X-Tyr-Glu-Glu-Glu-X). The helix represents a deviation from the regular β-sheet architecture of the β-propeller domain (FIGS. 1 and 3A). The superposition of the active sites of the exopeptidase DPP-IV complexed with Diprotin A and the endopeptidase POP complexed with an octapeptide (Fülop, V., Szeltner, Z., Renner, V. & Polgar, L. (2001). Structures of prolyl oligopeptidase substrate/inhibitor complexes. Use of inhibitor binding for titration of the catalytic histidine residue. J. Biol. Chem. 276, 1262-1266) shows clear differences. The octapeptide substrate of POP coincides with the double Glu-motif in DPP-IV indicating that this additional structural element functions is very important for substrate selection. Thus, the double Glu-motif is a recognition site for the N-terminus of substrates and restricts the cleavage to dipeptides and the S1 pocket provides an optimal binding to proline and alanine residues leading to a highly specific peptidase.

Mode of Inhibition by Diprotin-A

Inspection of the electron density of the bound inhibitor shows a covalent linkage to Ser630 and a sp³-configuration for the C-atom of the former carbonyl-group of the scissile peptide. Consequently, a tetrahedral intermediate is observed in the complex structure with the substrate Diprotin A (FIG. 5) with the oxyanion stabilized by hydrogen bonds to the hydroxy group of the side chain of Tyr547 (2.80 Å) and the main chain amine of Tyr631 (3.38 Å). As much catalytic power of serine proteases derives from its preferential binding of this transition state, the tetrahedral intermediate is a well-defined but high energy state with a short lifetime and its accumulation must be a result of a kinetic barrier.

Inspection of the active site structure reveals several structural features that are special to Diprotin A and may lead to the competitive inhibition of this substrate. First, the two hydrophobic isoleucine side chains point into the same direction in proximity and, therefore, this hydrophobic interaction may stabilize the tripeptide in a unsuitable conformation for the progress of the reaction. Second, a large network of salt bridges and hydrogen bonds stabilize the complex. It involves the carboxyl groups of Glu205/206 that interact with the N-terminus of the tripeptide, but Glu205 makes another salt bridge to Arg125 and this in turn interacts with the C-terminal carboxyl group of the tripeptide (FIG. 5). It is obvious that this interaction is only present in tripeptidic substrates and may stabilize the observed intermediate by protection of the leaving group.

Dimerization

The crystal structure as well as analytical ultracentrifugation indicate dimeric oligomerization for deglycosylated sDPP-IV with a molecular weight of 169 kDa and non-crystallographic twofold symmetry (FIG. 2). Six percent or 1837 Å² of the total solvent accessible surface area of each subunit is buried in the dimer interface (program XSAE, Broger, C. personal communication). This interface is mainly build up by two extra β-strands (β1* and β2*) in the loop between the strands two and three of the fourth blade of the β-propeller domain (FIGS. 3A and 3B). Further interaction is provided by the α/β hydrolase domain with helix αE, β-strand β8 and helix αF with mainly hydrophobic interactions. The active site is very close to this dimer interface (FIG. 2) with His740 from the catalytic triad located in the loop connecting αF and β7 (FIG. 1). Consequently disruption of the dimer interface would also strongly affect the catalytic activity and dimerization is required for activity.

Stability of DPP-IV

As a cell surface protein DPP-IV is extremely stable. Consequently the recombinant sDPP-IV shows a half life of 5 min at 71° C. in irreversible heat inactivation experiments independent of the protein concentration and the degree of glycosylation indicating high thermal stability. In unfolding experiments (Lambeir, A. M., Diaz Pereira, J. F., Chacon, P., Vermeulen, G., Heremans, K., Devreese, B., Van Beeumen, J., De Meester, I. & Scharpe, S. (1997). A prediction of DPP IV/CD26 domain structure from a physico-chemical investigation of dipeptidyl peptidase IV (CD26) from human seminal plasma. Biochim. Biophys. Acta 1340, 215-2) with protein purified from human seminal plasma DPP-IV retained its native conformation up to 8 M Urea.

The crystal structure points to several factors that may contribute to this stability. Firstly, the structural organization as a dimer with an extended hydrophobic interface stabilizes the molecule as shown for several other proteins (Thoma, R., Hennig, M., Sterner, R. & Kirschner, K. (2000). Structure and function of mutationally generated monomers of dimeric phosphoribosylanthranilate isomerase from Thermotoga maritima. Structure Fold. Des. 8, 265-276). Secondly, we observe five disulphide bonds and two free sulfhydryl groups by SH titration experiments under denaturing conditions that are now confirmed by the X-ray structure. All disulphide bridges in the β-propeller connect different strands in blades or stabilize loops (Cys444/Cys447; Cys385/Cys394, Cys454/Cys472, Cys328/Cys339). One disulfide bond is observed in the α/β-hydrolase domain (Cys649/Cys762) and covalently links the C-terminal helix αF to the core of the α/β hydrolase domain.

Glycosylation

sDPP-IV overexpressed in P. pastoris shows a decreasing molecular weight over the elution peak in the analytical gelfiltration as analyzed online with a multiangle laser light scattering detector. In contrast, sDPP-IV deglycosylated with EndoF glycosidase shows an uniform molecular weight over the whole peak range, because of the specific cleavage of asparagine linked oligomannose after the first N-acetylglucoamines residue (GlcNAc). This leads to a decrease in molecular weight of 20 kDa as estimated by SDS-PAGE. Crystals suitable for X-ray diffraction are only observed for deglycosylated sDPP-IV and structure analysis shows four GlcNAc with interpretable electron density at the positions N85, N150, N229 and N281 in subunit A. In subunit B, again N85, N150 and N229 are visible, but no electron density was found for N281 and an additional site could be identified at N92. The GlcNAc of N85 is involved in a crystal contact in both subunits.

DPP-IV expressed in human has a more complex type of glycosylation compared to P. pastoris (Cremata, J., Montensino, R., Quintero, O. & Garcia, R. (1998). Glycosylation Profiling of Heterologous Proteins. In Pichia Protocols (Higgins, D. R. & Cregg, J. M., eds.), vol. 103. pp. 95-106, Humana Press: Totowa, N. J.) and contains terminal sialic acid, however, this seems not to be a requirement for correct folding as shown here.

Interaction with ADA

Adenosine deaminase (ADA; EC 3.5.4.4) is a 41 kDa protein expressed in all mammaliantissues that catalyzes the deamidation of adenosine and 2′-deoxyadenosine to inosine and 2′-deoxyinosine, respectively. It is important for the regulation of the extracellular concentration of adenosine and for the regulation of the immune response. ADA is involved in T cell activation in general and the pathogensis of auto immune disorders (such as rheumatoid arthritis) as well as the mechanism of immunodeficiency disease (such as SCID or AIDS). Binding of the soluble extracellular ADA is a unique property of DPP-IV molecules of higher mammals and is not observed in mouse nor rat DPP-IV (Iwaki-Egawa, S., Watanabe, Y. & Fujimoto, Y. (1997). CD26/dipeptidyl peptidase IV does not work as an adenosine deaminase-binding protein in rat cells. Cell Immunol. 178, 180-186). Using analytical ultra-centrifugation, we observe a 1:1 complex of a ADA molecules with a sDPP-IV subunit giving a molecular weight of 252 kDa. Surface plasmon resonance (Biacore) measurements show a binding constant of 3.15±2 nM to ADA from bovine with a very low dissociation rate (k_{off}=8.75*10⁻⁵s⁻¹, kₒₙ=2.98*10⁴M⁻¹s⁻¹) indicating a strong interaction.

Mutagenesis studies (Abbott, C. A., McCaughan, G. W., Levy, M. T., Church, W. B. & Gorrell, M. D. (1999). Binding to human dipeptidyl peptidase IV by adenosine deaminase and antibodies that inhibit ligand binding involves overlapping, discontinuous sites on a predicted beta propeller domain. Eur. J. Biochem. 266, 798-810; Dong, R. P., Tachibana, K., Hegen, M., Munakata, Y., Cho, D., Schlossman, S. F. & Morimoto, C. (1997). Determination of adenosine deaminase binding domain on CD26 and its immunoregulatory effect on T cell activation. J. Immunol. 159, 6070-6076) identified two important regions in DPP-IV Leu₃₄₀-Val₃₄₁-Ala₃₄₂-Arg₃₄₃ (at the beginning of β5/4) and Leu294 (α4, at the end of blade 4) and a less important region Glu₃₃₂-Ser₃₃₃-Ser₃₃₄-Gly₃₃₅-Arg₃₃₆ (loop region, at the end of β5/3) that are all located at the surface of the β-propeller domain (FIG. 1). Mutation to amino acids found in rat DPP-IV reduces binding affinity to ADA. These residues form a binding site that is located far away from the active site (FIG. 2) confirming the independence of DPP-IV activity on ADA binding (Table 1; De Meester, I., Vanham, G., Kestens, L., Vanhoof, G., Bosmans, E., Gigase, P. & Scharpe, S. (1994). Binding of adenosine deaminase to the lymphocyte surface via CD26. Eur. J. Immunol. 24, 566-570). It is concluded that the function of DPP-IV is the localization and orientation of ADA for proper catalysis. The structure gives an indication for the orientation and localization at the cell surface, because the N-terminus must be close to the membrane and the ADA binding would be on the opposite site of the molecule-pointing away from the cell surface (FIG. 2). Further, there would be sufficient space enabling interaction of ADA to the A1-adenosine receptor (Ciruela, F., Saura, C., Canela, E. I., Mallol, J., Lluis, C. & Franco, R. (1996). Adenosine deaminase affects ligand-induced signaling by interacting with cell surface adenosine receptors. FEBS Lett. 380, 219-223) which probably plays an important role in the ontogenesis of immune tissues. This view would also support the hypothesis proposing a link for cell-cell interaction via the binding of DPP-IV, ADA and A1-adenosine.

Biological Implications

The crystal structure of DPP-IV at 2.1 Å resolution reveals a V-shaped dimeric molecule with an extended dimer interface fostering the conformation of the overall molecule. The membrane association and stability of DPP-IV is used for binding of other proteins like ADA in order to achieve localization without disturbance of the enzymatic functionality.

Analysis of the complex with Diprotin A shows key structural features for proline specific exopeptidase specificity and activity. The negative charge of the double Glu motif guides the N-terminus of the peptide to the active site and fixes the substrate in the correct position for cleavage. The distance between this motif and the catalytic Ser630 limits the cleavage to dipeptides and the S1 pocket can just adopt proline or with less affinity alanine as side chains.

The low turnover rate of Diprotin A may be explained by the hydrophobic interaction of the two Ile-residues in the P2 and P1′ positions as well as an extensive salt bridge cluster that involves the negatively charged C-terminus of Diprotin A. This structural information will aid the design of new specific inhibitors.

The active site is very accessible to the solvent by two entrances explaining that peptides can be cleaved by DPP-IV with almost no size limitation. A second access to the active site by the tunnel of the β-propeller domain is large enough to enable the release of the cleaved dipeptides. This structural arrangement certainly improves the catalytic turnover and is in great contrast to the crystal structure of POP that shows a much more narrow tunnel and no further access to the active site.

For most of the special features of DPP-IV namely dimerization, regulation of substrate access via two entrances, recognition of the substrate (double Glu-motif) and interaction with other proteins like ADA the β-propeller domain plays a key role. Thus, DPP-IV is an excellent example that the β-propeller fold can be tailored to adapt to different functionality.

TABLE 1
Enzyme Kinetic Constants of DPP-IV

| | k_{cat}* | K_{M}* | k_{cat}/K_{M} |
|---|---|---|---|
| proteins | (s⁻¹) | μM) | μM⁻¹s⁻¹) |
| sDPP-IV_{deglycos} | 43.1 | 17.2 | 2.51 |
| sDPP-IV_{glycos} | 37.3 | 15.5 | 2.41 |
| sDPP-IV_{deglycos.}/ADA | 39.6 | 14.8 | 2.68 |

*analyzed using Lineweaver-Burk plots; buffer: 50 mM Tris/HCl pH 7.8, containing 100 mM NaCl, 0.1 mg/ml BSA and 0.5% Dimethyl-formamid; temperature: 25° C.

TABLE 2
K_{I} and K_{D} Values of DPP-IV Inhibitors

| | K_{I} | K_{D} | kₒₙ | k_{off} |
|---|---|---|---|---|
| | μM) | μM) | M⁻¹s⁻¹ | s⁻¹ |
| Ile-Pro-Ile | 4.63^{‡} | 3.8^{†} | — | — |
| NVP-DPP728 | 0.006^{‡} | 0.002^{†} | 1.36 * 10^{6†} | 2.48 * 10^{-3†} |
| NVP-DPP728_{(Lit.)}* | 0.011 | 0.010 | 1.3 * 10⁵ | 1.3 * 10⁻³ |

^{†}measured with biacore; buffer: 0.01 M Hepes, pH 7.4, containing 0.15 M NaCl, 3 mM EDTA, 0.005% polysorbate 20 (v/v)
^{‡}temperature: 25° C.; in assay buffer (see Table 1); glycosylated sDPP-IV
*Hughes, T. E., Mone, M. D., Russell, M. E., Weldon, S. C. & Villhauer, E. B. (1999).
NVP-DPP728 (1-[[[2-[(5-cyanopyridin-2-yl)amino]ethyl]amino]acetyl]-2-cyano-(S)-pyrrolidine), a slow-binding inhibitor of dipeptidyl peptidase IV. Biochemistry 38, 11597-11603

TABLE 3
Crystallographic Data and Refinement Statistics

| | MAD | MAD | MAD | | Diprotin-A |
|---|---|---|---|---|---|
| Data set | Remote | Peak | Inflection | Apo | complex |
| Wavelength | 0.992 | 1.0065 | 1.009 | 0.9765 | 0.92 |
| X-ray source | SLS | SLS | SLS | ID14, ESRF | SLS |
| Detector | MAR IP^{a} | MAR IP^{a} | MAR IP^{a} | Quantum | MAR CCD |
| | | | | CCD | |
| Exposure time/frame (s) | 10 | 10 | 10 | 2 | 4 |
| angular increment per frame (°) | 2.0 | 2.0 | 2.0 | 0.25 | 0.25 |
| total rotation range (°) | 110 | 136 | 140 | 130 | 130 |
| crystal to detector distance (mm) | 410 | 410 | 410 | 240 | 260 |
| unit cell parameters a, b, c (Å) | 65.2; 68.7; | 65.2; 68.7; | 65.2; 68.7; | 65.5; 68.2; | 65.1; 67.1; |
| | 420.1 | 420.1 | 420.1 | 419.3 | 419.6 |

data reduction

| | | | | | |
|---|---|---|---|---|---|
| Maximum Resolution (Å) | 2.6 | 2.6 | 2.6 | 2.1 | 2.5 |
| No. of measurements | 212 619 | 263 910 | 276 921 | 234 528 | 171 090 |
| No. of unique reflections | 58 627 | 59 544 | 59 939 | 87 113 | 64 208 |
| completeness (%)* | 97.5 (99.4) | 99.9 (100.0) | 99.9 (99.9) | 82.9 (72.3) | 97.5 (99.4) |
| Rsym*,^{b*} | 9.1 (15.9) | 9.0 (18.1) | 8.6 (14.2) | 8.4 (26.8) | 9.1 (15.9) |

heavy-atom refinement paramet

| | | | |
|---|---|---|---|
| f′(e)/f′ (e) | −7.0/9.5 | −8.0/9.8 | −12.1/5.0 |
| Phasing power^{c} (anomalous) | 0.95 | 1.0 | 0.7 |

Refinement statistics

| | | |
|---|---|---|
| resolution range (Å) | 20-2.1 | 30-2.5 |
| R_{cryst} (R_{free})^{d} (%) | 21.5 (26.5) | 22.5 (28.2) |
| No. of protein atoms^{c} (mean B | 11 962 | 11 962 |
| in Å²) | (34.6) | (27.1) |
| No. of water molecules | 322 (33.4) | 268 (25.0) |
| No. of ligand/heavy atoms | 6 (77.3) | 24 (28.3) |
| (mean B in Å²) | | |
| No. of NAG atoms (mean B in | 112 (59.0) | 98 (51.4) |
| Å²) | | |
| rmsd^{f} bonds (Å²) | 0.018 | 0.019 |
| Rmsd^{f} angles (°) | 1.86 | 2.07 |

^{a}Marresearch image plate detector, diameter 345 mm, 100 μm pixel size Values in parentheses are statistics for highest resolution bin.
^{b}R_{sym} = ΣₕΣᵢ|Iᵢ(h) − <I(h)>|/ΣₕΣᵢ(h), where Iᵢ(h) und <I(h)> are the ith and mean measurement of the intensity of reflection h.
^{c}Phasing power = ΣₕF_{H}(h)/Σₕ|F_{D}(h) − |F_{N}(h) + F_{H}(h)||.
^{d}Σₕ||F_{obs}| − |F_{calc}||/Σₕ|F_{obs}|, where |F_{obs}| and |F_{calc}| are the observed and calculated structure factor amplitudes for the reflection h, applied to the working (R_{cryst}) and test (R_{free})sets, respectively.
^{e}Non-hydrogen atoms, only.
^{f}rmsd: root mean square deviation from mean.

TABLE 4
Structure coordinates for human DPP-IV
Table 4 lists the atomic structure coordinates for DPP-IV as derived by X-ray
diffraction from a crystal of DPP-IV.

| HEADER | | DPP-IV |
|---|---|---|
| COMPND | | Human Dipeptidyl peptidase IV |
| COMPND | | |
| SOURCE | | human |
| REMARK | 1 | |
| REMARK | 1 | REFINEMENT REMARKS: |
| REMARK | 1 | |
| REMARK | 1 | |
| REMARK | 1 | “apo”-structure |
| REMARK | 1 | (mercury derivative different from MAD experiment used for |

refinement)

| REMARK | 1 | | |
|---|---|---|---|
| REMARK | 1 | | |
| REMARK | 2 | | |
| REMARK | 2 | 2.1A resolution | |
| REMARK | 2 | | |
| REMARK | 3 | | |
| REMARK | 3 | REFINEMENT. | |
| REMARK | 3 | PROGRAM | : REFMAC 5.0 |
| REMARK | 3 | AUTHORS | : MURSHUDOV, VAGIN, DODSON |
| REMARK | 3 | | |

| REMARK | 3 | REFINEMENT TARGET: MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) : | 2.10 |
| REMARK | 3 | RESOLUTION RANGE LOW | (ANGSTROMS) : | 12.00 |
| REMARK | 3 | DATA CUTOFF | (SIGMA (F)) : | NONE |
| REMARK | 3 | COMPLETENESS FOR RANGE | (%) : | 82.99 |
| REMARK | 3 | NUMBER OF REFLECTIONS | : | 87113 |
| REMARK | 3 | | | |

| REMARK | 3 | FIT TO DATA USED IN REFINEMENT. | |
|---|---|---|---|
| REMARK | 3 | CROSS-VALIDATION METHOD | : THROUGHOUT |
| REMARK | 3 | FREE R VALUE TEST SET SELECTION | : RANDOM |
| REMARK | 3 | R VALUE (WORKING + TEST SET) | : 0.21747 |
| REMARK | 3 | R VALUE (WORKING SET) | : 0.21485 |
| REMARK | 3 | FREE R VALUE | : 0.26560 |
| REMARK | 3 | FREE R VALUE TEST SET SIZE (%) | : 5.0 |
| REMARK | 3 | FREE R VALUE TEST SET COUNT | : 4619 |
| REMARK | 3 | | |
| REMARK | 3 | FIT IN THE HIGHEST RESOLUTION BIN. | |
| REMARK | 3 | TOTAL NUMBER OF BINS USED | : 20 |
| REMARK | 3 | BIN RESOLUTION RANGE HIGH | : 2.100 |
| REMARK | 3 | BIN RESOLUTION RANGE LOW | : 2.153 |
| REMARK | 3 | REFLECTION IN BIN (WORKING SET) | : 2014 |
| REMARK | 3 | BIN R VALUE (WORKING SET) | : 0.246 |
| REMARK | 3 | BIN FREE R VALUE SET COUNT | : 91 |
| REMARK | 3 | BIN FREE R VALUE | : 0.278 |
| REMARK | 3 | | |

| REMARK | 3 | NUMBER OF NON-HYDROGEN ATOMS USED IN REFINEMENT. | | |
|---|---|---|---|---|
| REMARK | 3 | ALL ATOMS : 12366 | | |
| REMARK | 3 | | | |
| REMARK | 3 | ESTIMATED OVERALL COORDINATE ERROR. | | |
| REMARK | 3 | ESU BASED ON R VALUE | (A): | 0.280 |
| REMARK | 3 | ESU BASED ON FREE R VALUE | (A): | 0.228 |
| REMARK | 3 | ESU BASED ON MAXIMUM LIKELIHOOD | (A): | 0.244 |
| REMARK | 3 | ESU FOR B VALUES BASED ON MAXIMUM LIKELIHOOD | (A**2): | 9.427 |
| REMARK | 3 | | | |
| REMARK | 3 | | | |

| REMARK | 3 | RMS DEVIATIONS FROM IDEAL VALUES | | COUNT | RMS | WEIGHT |
|---|---|---|---|---|---|---|
| REMARK | 3 | BOND LENGTHS REFINED ATOMS | (A): | 12400 ; | 0.018 ; | 0.021 |
| REMARK | 3 | BOND LENGTHS OTHERS | (A): | 10588 ; | 0.001 ; | 0.020 |
| REMARK | 3 | BOND ANGLES REFINED ATOMS | (DEGREES): | 16876 ; | 1.867 ; | 1.936 |
| REMARK | 3 | BOND ANGLES OTHERS | (DEGREES): | 24632 ; | 0.889 ; | 3.000 |
| REMARK | 3 | TORSION ANGLES, PERIOD 1 | (DEGREES): | 1454 ; | 5.183 ; | 3.000 |
| REMARK | 3 | TORSION ANGLES, PERIOD 3 | (DEGREES): | 2075 ; | 19.350 ; | 15.000 |
| REMARK | 3 | CHIRAL-CENTER RESTRAINTS | (A**3): | 1790 ; | 0.135 ; | 0.200 |
| REMARK | 3 | GENERAL PLANES REFINED ATOMS | (A): | 13738 ; | 0.007 ; | 0.020 |
| REMARK | 3 | GENERAL PLANES OTHERS | (A): | 2674 ; | 0.004 ; | 0.020 |
| REMARK | 3 | NON-BONDED CONTACTS REFINED ATOMS | (A): | 2592 ; | 0.240 ; | 0.300 |
| REMARK | 3 | NON-BONDED CONTACTS OTHERS | (A): | 10721 ; | 0.223 ; | 0.300 |
| REMARK | 3 | NON-BONDED TORSION OTHERS | (A): | 17 ; | 0.494 ; | 0.500 |
| REMARK | 3 | H-BOND (X . . . Y) REFINED ATOMS | (A): | 820 ; | 0.155 ; | 0.500 |
| REMARK | 3 | H-BOND (X . . . Y) OTHERS | (A): | 7 ; | 0.115 ; | 0.500 |
| REMARK | 3 | SYMMETRY VDW REFINED ATOMS | (A): | 9 ; | 0.235 ; | 0.300 |
| REMARK | 3 | SYMMETRY VDW OTHERS | (A): | 38 ; | 0.277 ; | 0.300 |
| REMARK | 3 | SYMMETRY H-BOND REFINED ATOMS | (A): | 3 ; | 0.397 ; | 0.500 |
| REMARK | 3 | | | | | |
| REMARK | 3 | ISOTROPIC THERMAL FACTOR RESTRAINTS. | | COUNT | RMS | WEIGHT |
| REMARK | 3 | MAIN-CHAIN BOND REFINED ATOMS | (A**2): | 7252 ; | 0.874 ; | 1.500; |
| REMARK | 3 | MAIN-CHAIN ANGLE REFINED ATOMS | (A**2): | 11766 ; | 1.603 ; | 2.000 |
| REMARK | 3 | SIDE-CHAIN BOND REFINED ATOMS | (A**2): | 5148 ; | 2.300 ; | 3.000 |
| REMARK | 3 | SIDE-CHAIN ANGLE REFINED ATOMS | (A**2): | 5110 ; | 3.638 ; | 4.500 |
| REMARK | 3 | | | | | |
| REMARK | 3 | NCS RESTRAINTS STATISTICS | | | | |
| REMARK | 3 | NUMBER OF NCS GROUPS: NULL | | | | |
| REMARK | 3 | | | | | |
| REMARK | 3 | | | | | |
| REMARK | 3 | | | | | |

| REMARK | 4 | data collected at 100K at ID14 in Grenoble (ESRF, France) | | | | | | | | | | | | | | |
|---|---|---|---|---|---|---|---|---|---|---|---|---|---|---|---|---|
| REMARK | 4 | Phasing by MAD using Hg derivative and data collected to 2.7 A | | | | | | | | | | | | | | |
| REMARK | 4 | at Villigen (SLS, Switzerland) | | | | | | | | | | | | | | |
| REMARK | 4 | | | | | | | | | | | | | | | |
| SEQRES | 1 | A | 728 | SER | ARG | LYS | THR | TYR | THR | LEU | THR | ASP | TYR | LEU | LYS | ASN |
| SEQRES | 2 | A | 728 | THR | TYR | ARG | LEU | LYS | LEU | TYR | SER | LEU | ARG | TRP | ILE | SER |
| SEQRES | 3 | A | 728 | ASP | HIS | GLU | TYR | LEU | TYR | LYS | GLN | GLU | ASN | ASN | ILE | LEU |
| SEQRES | 4 | A | 728 | VAL | PHE | ASN | ALA | GLU | TYR | GLY | ASN | SER | SER | VAL | PHE | LEU |
| SEQRES | 5 | A | 728 | GLU | ASN | SER | THR | PHE | ASP | GLU | PHE | GLY | HIS | SER | ILE | ASN |
| SEQRES | 6 | A | 728 | ASP | TYR | SER | ILE | SER | PRO | ASP | GLY | GLN | PHE | ILE | LEU | LEU |
| SEQRES | 7 | A | 728 | GLU | TYR | ASN | TYR | VAL | LYS | GLN | TRP | ARG | HIS | SER | TYR | THR |
| SEQRES | 8 | A | 728 | ALA | SER | TYR | ASP | ILE | TYR | ASP | LEU | ASN | LYS | ARG | GLN | LEU |
| SEQRES | 9 | A | 728 | ILE | THR | GLU | GLU | ARG | ILE | PRO | ASN | ASN | THR | GLN | TRP | VAL |
| SEQRES | 10 | A | 728 | THR | TRP | SER | PRO | VAL | GLY | HIS | LYS | LEU | ALA | TYR | VAL | TRP |
| SEQRES | 11 | A | 728 | ASN | ASN | ASP | ILE | TYR | VAL | LYS | ILE | GLU | PRO | ASN | LEU | PRO |
| SEQRES | 12 | A | 728 | SER | TYR | ARG | ILE | THR | TRP | THR | GLY | LYS | GLU | ASP | ILE | ILE |
| SEQRES | 13 | A | 728 | TYR | ASN | GLY | ILE | THR | ASP | TRP | VAL | TYR | GLU | GLU | GLU | VAL |
| SEQRES | 14 | A | 728 | PHE | SER | ALA | TYR | SER | ALA | LEU | TRP | TRP | SER | PRO | ASN | GLY |
| SEQRES | 15 | A | 728 | THR | PHE | LEU | ALA | TYR | ALA | GLN | PHE | ASN | ASP | THR | GLU | VAL |
| SEQRES | 16 | A | 728 | PRO | LEU | ILE | GLU | TYR | SER | PHE | TYR | SER | ASP | GLU | SER | LEU |
| SEQRES | 17 | A | 728 | GLN | TYR | PRO | LYS | THR | VAL | ARG | VAL | PRO | TYR | PRO | LYS | ALA |
| SEQRES | 18 | A | 728 | GLY | ALA | VAL | ASN | PRO | THR | VAL | LYS | PHE | PHE | VAL | VAL | ASN |
| SEQRES | 19 | A | 728 | THR | ASP | SER | LEU | SER | SER | VAL | THR | ASN | ALA | THR | SER | ILE |
| SEQRES | 20 | A | 728 | GLN | ILE | THR | ALA | PRO | ALA | SER | MET | LEU | ILE | GLY | ASP | HIS |
| SEQRES | 21 | A | 728 | TYR | LEU | CYS | ASP | VAL | THR | TRP | ALA | THR | GLN | GLU | ARG | ILE |
| SEQRES | 22 | A | 728 | SER | LEU | GLN | TRP | LEU | ARG | ARG | ILE | GLN | ASN | TYR | SER | VAL |
| SEQRES | 23 | A | 728 | MET | ASP | ILE | CYS | ASP | TYR | ASP | GLU | SER | SER | GLY | ARG | TRP |
| SEQRES | 24 | A | 728 | ASN | CYS | LEU | VAL | ALA | ARG | GLN | HIS | ILE | GLU | MET | SER | THE |
| SEQRES | 25 | A | 728 | THR | GLY | TRP | VAL | GLY | ARG | PHE | ARG | PRO | SER | GLU | PRO | HIS |
| SEQRES | 26 | A | 728 | PHE | THR | LEU | ASP | GLY | ASN | SER | PHE | TYR | LYS | ILE | ILE | SER |
| SEQRES | 27 | A | 728 | ASN | GLU | GLU | GLY | TYR | ARG | HIS | ILE | CYS | TYR | PHE | GLN | ILE |
| SEQRES | 28 | A | 728 | ASP | LYS | LYS | ASP | CYS | THR | PHE | ILE | THR | LYS | GLY | THR | TRP |
| SEQRES | 29 | A | 728 | GLU | VAL | ILE | GLY | ILE | GLU | ALA | LEU | THR | SER | ASP | TYR | LEU |
| SEQRES | 30 | A | 728 | TYR | TYR | ILE | SER | ASN | GLU | TYR | LYS | GLY | MET | PRO | GLY | GLY |
| SEQRES | 31 | A | 728 | ARG | ASN | LEU | TYR | LYS | ILE | GLN | LEU | SER | ASP | TYR | THR | LYS |
| SEQRES | 32 | A | 728 | VAL | THR | CYS | LEU | SER | CYS | GLU | LEU | ASN | PRO | GLU | ARG | CYS |
| SEQRES | 33 | A | 728 | GLN | TYR | TYR | SER | VAL | SER | PHE | SER | LYS | GLU | ALA | LYS | TYR |
| SEQRES | 34 | A | 728 | TYR | GLN | LEU | ARG | CYS | SER | GLY | PRO | GLY | LEU | PRO | LEU | TYR |
| SEQRES | 35 | A | 728 | THR | LEU | HIS | SER | SER | VAL | ASN | ASP | LYS | GLY | LEU | ARG | VAL |
| SEQRES | 36 | A | 728 | LEU | GLU | ASP | ASN | SER | ALA | LEU | ASP | LYS | MET | LEU | GLN | ASN |
| SEQRES | 37 | A | 728 | VAL | GLN | MET | PRO | SER | LYS | LYS | LEU | ASP | PHE | ILE | ILE | LEU |
| SEQRES | 38 | A | 728 | ASN | GLU | THR | LYS | PHE | TRP | TYR | GLN | MET | ILE | LEU | PRO | PRO |
| SEQRES | 39 | A | 728 | HIS | PHE | ASP | LYS | SER | LYS | LYS | TYR | PRO | LEU | LEU | LEU | ASP |
| SEQRES | 40 | A | 728 | VAL | TYR | ALA | GLY | PRO | CYS | SER | GLN | LYS | ALA | ASP | THR | VAL |
| SEQRES | 41 | A | 728 | PHE | ARG | LEU | ASN | TRP | ALA | THR | TYR | LEU | ALA | SER | THR | GLU |
| SEQRES | 42 | A | 728 | ASN | ILE | ILE | VAL | ALA | SER | PHE | ASP | GLY | ARG | GLY | SER | GLY |
| SEQRES | 43 | A | 728 | TYR | GLN | GLY | ASP | LYS | ILE | MET | HIS | ALA | ILE | ASN | ARG | ARG |
| SEQRES | 44 | A | 728 | LEU | GLY | THR | PHE | GLU | VAL | GLU | ASP | GLN | ILE | GLU | ALA | ALA |
| SEQRES | 45 | A | 728 | ARG | GLN | PHE | SER | LYS | MET | GLY | PHE | VAL | ASP | ASN | LYS | ARG |
| SEQRES | 46 | A | 728 | ILE | ALA | ILE | TRP | GLY | TRP | SER | TYR | GLY | GLY | TYR | VAL | THR |
| SEQRES | 47 | A | 728 | SER | MET | VAL | LEU | GLY | SER | GLY | SER | GLY | VAL | PHE | LYS | CYS |
| SEQRES | 48 | A | 728 | GLY | ILE | ALA | VAL | ALA | PRO | VAL | SER | ARG | TRP | GLN | TYR | TYR |
| SEQRES | 49 | A | 728 | ASP | SER | VAL | TYR | THR | GLU | ARG | TYR | MET | GLY | LEU | PRO | THR |
| SEQRES | 50 | A | 728 | PRO | GLU | ASP | ASN | LEU | ASP | HIS | TYR | ARG | ASN | SER | THR | VAL |
| SEQRES | 51 | A | 728 | MET | SER | ARG | ALA | GLU | ASN | PHE | LYS | GLN | VAL | GLU | TYR | LEU |
| SEQRES | 52 | A | 728 | LEU | ILE | HIS | GLY | THR | ALA | ASP | ASP | ASN | VAL | HIS | PHE | GLN |
| SEQRES | 53 | A | 728 | GLN | SER | ALA | GLN | ILE | SER | LYS | ALA | LEU | VAL | ASP | VAL | GLY |
| SEQRES | 54 | A | 728 | VAL | ASP | PHE | GLN | ALA | MET | TRP | TYR | THR | ASP | GLU | ASP | HIS |
| SEQRES | 55 | A | 728 | GLY | ILE | ALA | SER | SER | THR | ALA | HIS | GLN | HIS | ILE | TYR | THR |
| SEQRES | 56 | A | 728 | HIS | MET | SER | HIS | PHE | ILE | LYS | GLN | CYS | PHE | SER | LEU | PRO |
| SEQRES | 1 | B | 728 | SER | ARG | LYS | THR | TYR | THR | LEU | THR | ASP | TYR | LEU | LYS | ASN |
| SEQRES | 2 | B | 728 | THR | TYR | ARG | LEU | LYS | LEU | TYR | SER | LEU | ARG | TRP | ILE | SER |
| SEQRES | 3 | B | 728 | ASP | HIS | GLU | TYR | LEU | TYR | LYS | GLN | GLU | ASN | ASN | ILE | LEU |
| SEQRES | 4 | B | 728 | VAL | PHE | ASN | ALA | GLU | TYR | GLY | ASN | SER | SER | VAL | PHE | LEU |
| SEQRES | 5 | B | 728 | GLU | ASN | SER | THR | PHE | ASP | GLU | PHE | GLY | HIS | SER | ILE | ASN |
| SEQRES | 6 | B | 728 | ASP | TYR | SER | ILE | SER | PRO | ASP | GLY | GLN | PHE | ILE | LEU | LEU |
| SEQRES | 7 | B | 728 | GLU | TYR | ASN | TYR | VAL | LYS | GLN | TRP | ARG | HIS | SER | TYR | THR |
| SEQRES | 8 | B | 728 | ALA | SER | TYR | ASP | ILE | TYR | ASP | LEU | ASN | LYS | ARG | GLN | LEU |
| SEQRES | 9 | B | 728 | ILE | THR | GLU | GLU | ARG | ILE | PRO | ASN | ASN | THR | GLN | TRP | VAL |
| SEQRES | 10 | B | 728 | THR | TRP | SER | PRO | VAL | GLY | HIS | LYS | LEU | ALA | TYR | VAL | TRP |
| SEQRES | 11 | B | 728 | ASN | ASN | ASP | ILE | TYR | VAL | LYS | ILE | GLU | PRO | ASN | LEU | PRO |
| SEQRES | 12 | B | 728 | SER | TYR | ARG | ILE | THR | TRP | THR | GLY | LYS | GLU | ASP | ILE | ILE |
| SEQRES | 13 | B | 728 | TYR | ASN | GLY | ILE | THR | ASP | TRP | VAL | TYR | GLU | GLU | GLU | VAL |
| SEQRES | 14 | B | 728 | PHE | SER | ALA | TYR | SER | ALA | LEU | TRP | TRP | SER | PRO | ASN | GLY |
| SEQRES | 15 | B | 728 | THR | PHE | LEU | ALA | TYR | ALA | GLN | PHE | ASN | ASP | THR | GLU | VAL |
| SEQRES | 16 | B | 728 | PRO | LEU | ILE | GLU | TYR | SER | PHE | TYR | SER | ASP | GLU | SER | LEU |
| SEQRES | 17 | B | 728 | GLN | TYR | PRO | LYS | THR | VAL | ARG | VAL | PRO | TYR | PRO | LYS | ALA |
| SEQRES | 18 | B | 728 | GLY | ALA | VAL | ASN | PRO | THR | VAL | LYS | PHE | PHE | VAL | VAL | ASN |
| SEQRES | 19 | B | 728 | THR | ASP | SER | LEU | SER | SER | VAL | THR | ASN | ALA | THR | SER | ILE |
| SEQRES | 20 | B | 728 | GLN | ILE | THR | ALA | PRO | ALA | SER | MET | LEU | ILE | GLY | ASP | HIS |
| SEQRES | 21 | B | 728 | TYR | LEU | CYS | ASP | VAL | THR | TRP | ALA | THR | GLN | GLU | ARG | ILE |
| SEQRES | 22 | B | 728 | SER | LEU | GLN | TRP | LEU | ARG | ARG | ILE | GLN | ASN | TYR | SER | VAL |
| SEQRES | 23 | B | 728 | MET | ASP | ILE | CYS | ASP | TYR | ASP | GLU | SER | SER | GLY | ARG | TRP |
| SEQRES | 24 | B | 728 | ASN | CYS | LEU | VAL | ALA | ARG | GLN | HIS | ILE | GLU | MET | SER | TER |
| SEQRES | 25 | B | 728 | THR | GLY | TRP | VAL | GLY | ARG | PHE | ARG | PRO | SER | GLU | PRO | HIS |
| SEQRES | 26 | B | 728 | PHE | THR | LEU | ASP | GLY | ASN | SER | PHE | TYR | LYS | ILE | ILE | SER |
| SEQRES | 27 | B | 728 | ASN | GLU | GLU | GLY | TYR | ARG | HIS | ILE | CYS | TYR | PHE | GLN | ILE |
| SEQRES | 28 | B | 728 | ASP | LYS | LYS | ASP | CYS | THR | PHE | ILE | THR | LYS | GLY | THR | TRP |
| SEQRES | 29 | B | 728 | GLU | VAL | ILE | GLY | ILE | GLU | ALA | LEU | THR | SER | ASP | TYR | LEU |
| SEQRES | 30 | B | 728 | TYR | TYR | ILE | SER | ASN | GLU | TYR | LYS | GLY | MET | PRO | GLY | GLY |
| SEQRES | 31 | B | 728 | ARG | ASN | LEU | TYR | LYS | ILE | GLN | LEU | SER | ASP | TYR | THR | LYS |
| SEQRES | 32 | B | 728 | VAL | THR | CYS | LEU | SER | CYS | GLU | LEU | ASN | PRO | GLU | ARG | CYS |
| SEQRES | 33 | B | 728 | GLN | TYR | TYR | SER | VAL | SER | PHE | SER | LYS | GLU | ALA | LYS | TYR |
| SEQRES | 34 | B | 728 | TYR | GLN | LEU | ARG | CYS | SER | GLY | PRO | GLY | LEU | PRO | LEU | TYR |
| SEQRES | 35 | B | 728 | THR | LEU | HIS | SER | SER | VAL | ASN | ASP | LYS | GLY | LEU | ARG | VAL |
| SEQRES | 36 | B | 728 | LEU | GLU | ASP | ASN | SER | ALA | LEU | ASP | LYS | MET | LEU | GLN | ASN |
| SEQRES | 37 | B | 728 | VAL | GLN | MET | PRO | SER | LYS | LYS | LEU | ASP | PHE | ILE | ILE | LEU |
| SEQRES | 38 | B | 728 | ASN | GLU | THR | LYS | PHE | TRP | TYR | GLN | MET | ILE | LEU | PRO | PRO |
| SEQRES | 39 | B | 728 | HIS | PHE | ASP | LYS | SER | LYS | LYS | TYR | PRO | LEU | LEU | LEU | ASP |
| SEQRES | 40 | B | 728 | VAL | TYR | ALA | GLY | PRO | CYS | SER | GLN | LYS | ALA | ASP | THR | VAL |
| SEQRES | 41 | B | 728 | PHE | ARG | LEU | ASN | TRP | ALA | THR | TYR | LEU | ALA | SER | THR | GLU |
| SEQRES | 42 | B | 728 | ASN | ILE | ILE | VAL | ALA | SER | PHE | ASP | GLY | ARG | GLY | SER | GLY |
| SEQRES | 43 | B | 728 | TYR | GLN | GLY | ASP | LYS | ILE | MET | HIS | ALA | ILE | ASN | ARG | ARG |
| SEQRES | 44 | B | 728 | LEU | GLY | THR | PHE | GLU | VAL | GLU | ASP | GLN | ILE | GLU | ALA | ALA |
| SEQRES | 45 | B | 728 | ARG | GLN | PHE | SER | LYS | MET | GLY | PHE | VAL | ASP | ASN | LYS | ARG |
| SEQRES | 46 | B | 728 | ILE | ALA | ILE | TRP | GLY | TRP | SER | TYR | GLY | GLY | TYR | VAL | THR |
| SEQRES | 47 | B | 728 | SER | MET | VAL | LEU | GLY | SER | GLY | SER | GLY | VAL | PHE | LYS | CYS |
| SEQRES | 48 | B | 728 | GLY | ILE | ALA | VAL | ALA | PRO | VAL | SER | ARG | TRP | GLU | TYR | TYR |
| SEQRES | 49 | B | 728 | ASP | SER | VAL | TYR | THR | GLU | ARG | TYR | MET | GLY | LEU | PRO | TER |
| SEQRES | 50 | B | 728 | PRO | GLU | ASP | ASN | LEU | ASP | HIS | TYR | ARG | ASN | SER | THR | VAL |
| SEQRES | 51 | B | 728 | MET | SER | ARG | ALA | GLU | ASN | PHE | LYS | GLN | VAL | GLU | TYR | LEU |
| SEQRES | 52 | B | 728 | LEU | ILE | HIS | GLY | THR | ALA | ASP | ASP | ASN | VAL | HIS | PHE | GLN |
| SEQRES | 53 | B | 728 | GLN | SER | ALA | GLN | ILE | SER | LYS | ALA | LEU | VAL | ASP | VAL | GLY |
| SEQRES | 54 | B | 728 | VAL | ASP | PHE | GLN | ALA | MET | TRP | TYR | THR | ASP | GLU | ASP | HIS |
| SEQRES | 55 | B | 728 | GLY | ILE | ALA | SER | SER | THR | ALA | HIS | GLN | HIS | ILE | TYR | THR |
| SEQRES | 56 | B | 728 | HIS | MET | SER | HIS | PHE | ILE | LYS | GLN | CYS | PHE | SER | LEU | PRO |

| | | | | |
|---|---|---|---|---|
| HET | NAG | A | 793 | 14 |
| HET | NAG | A | 794 | 14 |
| HET | NAG | A | 795 | 14 |
| HET | NAG | A | 796 | 14 |
| HET | NAG | B | 793 | 14 |
| HET | NAG | B | 794 | 14 |
| HET | NAG | B | 796 | 14 |
| HET | NAG | B | 797 | 14 |
| HET | HG | Y | 303 | 1 |
| HET | HG | Y | 301 | 1 |
| HET | HG | Y | 302 | 1 |
| HET | HG | Z | 303 | 1 |
| HET | HG | Z | 301 | 1 |
| HET | HG | Z | 302 | 1 |

| | | | | | | |
|---|---|---|---|---|---|---|
| FORMUL | 6 | NAG | C8 | H15 | N1 | O5 |
| FORMUL | 7 | NAG | C8 | H15 | N1 | O5 |
| FORMUL | 8 | NAG | C8 | H15 | N1 | O5 |
| FORMUL | 9 | NAG | C8 | H15 | N1 | O5 |
| FORMUL | 10 | NAG | C8 | H15 | N1 | O5 |
| FORMUL | 11 | NAG | C8 | H15 | N1 | O5 |
| FORMUL | 12 | NAG | C8 | H15 | N1 | O5 |
| FORMUL | 13 | NAG | C8 | H15 | N1 | O5 |
| FORMUL | 14 | HG | HG1 | ++ | | |
| FORMUL | 15 | HG | HG1 | ++ | | |
| FORMUL | 16 | HG | HG1 | ++ | | |
| FORMUL | 17 | HG | HG1 | ++ | | |
| FORMUL | 18 | HG | HG1 | ++ | | |
| FORMUL | 19 | HG | HG1 | ++ | | |

| | | | |
|---|---|---|---|
| FORMUL | 20 | HOH | *322(H2 O1) |

| | | | | | | | | | | | |
|---|---|---|---|---|---|---|---|---|---|---|---|
| CRYST1 | 65.496 | 68.240 | 419.289 | 90.00 | 90.00 | 90.00 | P | 21 | 21 | 21 | 4 |

| | 2 | 3 | 4 | | | | | | | 10 |
|---|---|---|---|---|---|---|---|---|---|---|
| Cl. 1 | Atom | | Aa | | 5 | 6 | 7 | 8 | 9 | B |
| ATOM No. | type | | type | | Aa No. | X | Y | Z | OCC | factor |
| ATOM | 1 | N | SER | A | 39 | 81.432 | 37.048 | 22.064 | 1.00 | 53.58 |
| ATOM | 2 | CA | SER | A | 39 | 81.906 | 38.278 | 21.379 | 1.00 | 53.40 |
| ATOM | 3 | C | SER | A | 39 | 82.622 | 39.311 | 22.300 | 1.00 | 53.71 |
| ATOM | 4 | O | SER | A | 39 | 82.300 | 40.493 | 22.268 | 1.00 | 54.99 |
| ATOM | 5 | CB | SER | A | 39 | 80.683 | 38.903 | 20.729 | 1.00 | 53.66 |
| ATOM | 6 | OG | SER | A | 39 | 79.738 | 37.881 | 20.418 | 1.00 | 51.39 |
| ATOM | 7 | N | ARG | A | 40 | 83.591 | 38.872 | 23.109 | 1.00 | 53.25 |
| ATOM | 8 | CA | ARG | A | 40 | 84.264 | 39.750 | 24.098 | 1.00 | 52.31 |
| ATOM | 9 | C | ARG | A | 40 | 83.601 | 40.490 | 25.265 | 1.00 | 50.54 |
| ATOM | 10 | O | ARG | A | 40 | 83.314 | 39.903 | 26.298 | 1.00 | 49.96 |
| ATOM | 11 | CB | ARG | A | 40 | 85.768 | 39.965 | 23.920 | 1.00 | 52.68 |
| ATOM | 12 | CG | ARG | A | 40 | 86.628 | 38.946 | 24.740 | 1.00 | 54.78 |
| ATOM | 13 | CD | ARG | A | 40 | 85.794 | 37.897 | 25.546 | 1.00 | 57.24 |
| ATOM | 14 | NE | ARG | A | 40 | 86.328 | 36.534 | 25.499 | 1.00 | 58.28 |
| ATOM | 15 | CZ | ARG | A | 40 | 85.660 | 35.448 | 25.096 | 1.00 | 59.61 |
| ATOM | 16 | NH1 | ARG | A | 40 | 84.401 | 35.530 | 24.686 | 1.00 | 61.49 |
| ATOM | 17 | NH2 | ARG | A | 40 | 86.261 | 34.262 | 25.094 | 1.00 | 58.92 |
| ATOM | 18 | N | LYS | A | 41 | 83.456 | 41.803 | 25.081 | 1.00 | 48.23 |
| ATOM | 19 | CA | LYS | A | 41 | 82.818 | 42.756 | 25.984 | 1.00 | 46.53 |
| ATOM | 20 | C | LYS | A | 41 | 81.370 | 42.368 | 26.314 | 1.00 | 44.42 |
| ATOM | 21 | O | LYS | A | 41 | 80.703 | 41.655 | 25.573 | 1.00 | 43.94 |
| ATOM | 22 | CB | LYS | A | 41 | 82.863 | 44.105 | 25.282 | 1.00 | 46.80 |
| ATOM | 23 | CG | LYS | A | 41 | 82.277 | 45.301 | 25.964 | 1.00 | 48.40 |
| ATOM | 24 | CD | LYS | A | 41 | 81.868 | 46.280 | 24.842 | 1.00 | 48.84 |
| ATOM | 25 | CE | LYS | A | 41 | 82.184 | 47.736 | 25.157 | 1.00 | 51.33 |
| ATOM | 26 | NZ | LYS | A | 41 | 82.581 | 48.511 | 23.903 | 1.00 | 53.12 |
| ATOM | 27 | N | THR | A | 42 | 80.885 | 42.833 | 27.447 | 1.00 | 41.70 |
| ATOM | 28 | CA | THR | A | 42 | 79.609 | 42.354 | 27.944 | 1.00 | 39.43 |
| ATOM | 29 | C | THR | A | 42 | 78.630 | 43.494 | 28.003 | 1.00 | 37.10 |
| ATOM | 30 | O | THR | A | 42 | 79.076 | 44.650 | 27.942 | 1.00 | 36.72 |
| ATOM | 31 | CB | THR | A | 42 | 79.896 | 41.729 | 29.310 | 1.00 | 39.73 |
| ATOM | 32 | OG1 | THR | A | 42 | 79.355 | 40.410 | 29.352 | 1.00 | 40.73 |
| ATOM | 33 | CG2 | THR | A | 42 | 79.301 | 42.522 | 30.430 | 1.00 | 37.70 |
| ATOM | 34 | N | TYR | A | 43 | 77.317 | 43.203 | 28.044 | 1.00 | 34.78 |
| ATOM | 35 | CA | TYR | A | 43 | 76.299 | 44.283 | 28.125 | 1.00 | 33.64 |
| ATOM | 36 | C | TYR | A | 43 | 76.198 | 44.822 | 29.571 | 1.00 | 32.40 |
| ATOM | 37 | O | TYR | A | 43 | 75.706 | 44.146 | 30.453 | 1.00 | 29.56 |
| ATOM | 38 | CB | TYR | A | 43 | 74.918 | 43.829 | 27.617 | 1.00 | 33.75 |
| ATOM | 39 | CG | TYR | A | 43 | 73.894 | 44.942 | 27.562 | 1.00 | 32.19 |
| ATOM | 40 | CD1 | TYR | A | 43 | 73.804 | 45.770 | 26.453 | 1.00 | 31.74 |
| ATOM | 41 | CD2 | TYR | A | 43 | 72.986 | 45.146 | 28.603 | 1.00 | 31.84 |
| ATOM | 42 | CE1 | TYR | A | 43 | 72.874 | 46.782 | 26.373 | 1.00 | 30.93 |
| ATOM | 43 | CE2 | TYR | A | 43 | 72.047 | 46.157 | 28.533 | 1.00 | 29.54 |
| ATOM | 44 | CZ | TYR | A | 43 | 71.978 | 46.965 | 27.408 | 1.00 | 31.35 |
| ATOM | 45 | OH | TYR | A | 43 | 71.044 | 48.003 | 27.358 | 1.00 | 31.31 |
| ATOM | 46 | N | THR | A | 44 | 76.629 | 46.056 | 29.758 | 1.00 | 32.15 |
| ATOM | 47 | CA | THR | A | 44 | 76.897 | 46.588 | 31.100 | 1.00 | 33.67 |
| ATOM | 48 | C | THR | A | 44 | 75.766 | 47.433 | 31.694 | 1.00 | 32.40 |
| ATOM | 49 | O | THR | A | 44 | 74.842 | 47.835 | 30.988 | 1.00 | 31.22 |
| ATOM | 50 | CB | THR | A | 44 | 78.193 | 47.433 | 31.066 | 1.00 | 33.26 |
| ATOM | 51 | OG1 | THR | A | 44 | 79.329 | 46.619 | 30.661 | 1.00 | 39.41 |
| ATOM | 52 | CG2 | THR | A | 44 | 78.592 | 47.767 | 32.396 | 1.00 | 36.50 |
| ATOM | 53 | N | LEU | A | 45 | 75.859 | 47.711 | 32.989 | 1.00 | 31.13 |
| ATOM | 54 | CA | LEU | A | 45 | 74.864 | 48.531 | 33.618 | 1.00 | 30.66 |
| ATOM | 55 | C | LEU | A | 45 | 74.926 | 49.885 | 32.988 | 1.00 | 30.59 |
| ATOM | 56 | O | LEU | A | 45 | 73.880 | 50.426 | 32.631 | 1.00 | 30.36 |
| ATOM | 57 | CB | LEU | A | 45 | 75.080 | 48.633 | 35.113 | 1.00 | 31.36 |
| ATOM | 58 | CG | LEU | A | 45 | 74.141 | 49.585 | 35.847 | 1.00 | 30.22 |
| ATOM | 59 | CD1 | LEU | A | 45 | 72.682 | 49.261 | 35.589 | 1.00 | 32.27 |
| ATOM | 60 | CD2 | LEU | A | 45 | 74.430 | 49.492 | 37.345 | 1.00 | 29.39 |
| ATOM | 61 | N | THR | A | 46 | 76.132 | 50.425 | 32.818 | 1.00 | 29.60 |
| ATOM | 62 | CA | THR | A | 46 | 76.279 | 51.719 | 32.170 | 1.00 | 30.54 |
| ATOM | 63 | C | THR | A | 46 | 75.693 | 51.704 | 30.747 | 1.00 | 30.14 |
| ATOM | 64 | O | THR | A | 46 | 75.083 | 52.682 | 30.318 | 1.00 | 30.45 |
| ATOM | 65 | CB | THR | A | 46 | 77.758 | 52.173 | 32.102 | 1.00 | 30.74 |
| ATOM | 66 | OG1 | THR | A | 46 | 78.263 | 52.477 | 33.401 | 1.00 | 30.83 |
| ATOM | 67 | CG2 | THR | A | 46 | 77.855 | 53.537 | 31.420 | 1.00 | 32.16 |
| ATOM | 68 | N | ASP | A | 47 | 75.874 | 50.598 | 30.031 | 1.00 | 30.76 |
| ATOM | 69 | CA | ASP | A | 47 | 75.344 | 50.455 | 28.666 | 1.00 | 31.14 |
| ATOM | 70 | C | ASP | A | 47 | 73.841 | 50.702 | 28.685 | 1.00 | 31.39 |
| ATOM | 71 | O | ASP | A | 47 | 73.303 | 51.474 | 27.910 | 1.00 | 32.18 |
| ATOM | 72 | CB | ASP | A | 47 | 75.630 | 49.064 | 28.116 | 1.00 | 30.01 |
| ATOM | 73 | CG | ASP | A | 47 | 77.082 | 48.892 | 27.660 | 1.00 | 29.29 |
| ATOM | 74 | OD1 | ASP | A | 47 | 77.714 | 49.894 | 27.275 | 1.00 | 28.27 |
| ATOM | 75 | OD2 | ASP | A | 47 | 77.672 | 47.793 | 27.663 | 1.00 | 24.95 |
| ATOM | 76 | N | TYR | A | 48 | 73.179 | 50.083 | 29.643 | 1.00 | 31.92 |
| ATOM | 77 | CA | TYR | A | 48 | 71.745 | 50.232 | 29.770 | 1.00 | 31.38 |
| ATOM | 78 | C | TYR | A | 48 | 71.353 | 51.637 | 30.150 | 1.00 | 31.54 |
| ATOM | 79 | O | TYR | A | 48 | 70.493 | 52.230 | 29.567 | 1.00 | 29.15 |
| ATOM | 80 | CB | TYR | A | 48 | 71.264 | 49.307 | 30.847 | 1.00 | 31.04 |
| ATOM | 81 | CG | TYR | A | 48 | 69.857 | 49.596 | 31.233 | 1.00 | 28.68 |
| ATOM | 82 | CD1 | TYR | A | 48 | 68.846 | 49.629 | 30.284 | 1.00 | 27.94 |
| ATOM | 83 | CD2 | TYR | A | 48 | 69.554 | 49.874 | 32.529 | 1.00 | 29.63 |
| ATOM | 84 | CE1 | TYR | A | 48 | 67.534 | 49.907 | 30.636 | 1.00 | 32.09 |
| ATOM | 85 | CE2 | TYR | A | 48 | 68.242 | 50.126 | 32.930 | 1.00 | 31.86 |
| ATOM | 86 | CZ | TYR | A | 48 | 67.229 | 50.153 | 31.984 | 1.00 | 32.29 |
| ATOM | 87 | OH | TYR | A | 48 | 65.943 | 50.438 | 32.420 | 1.00 | 31.53 |
| ATOM | 88 | N | LEU | A | 49 | 72.020 | 52.160 | 31.155 | 1.00 | 32.84 |
| ATOM | 89 | CA | LEU | A | 49 | 71.725 | 53.485 | 31.669 | 1.00 | 34.60 |
| ATOM | 90 | C | LEU | A | 49 | 72.100 | 54.588 | 30.697 | 1.00 | 35.66 |
| ATOM | 91 | O | LEU | A | 49 | 71.456 | 55.643 | 30.660 | 1.00 | 34.88 |
| ATOM | 92 | CB | LEU | A | 49 | 72.533 | 53.695 | 32.944 | 1.00 | 34.83 |
| ATOM | 93 | CG | LEU | A | 49 | 71.926 | 53.503 | 34.334 | 1.00 | 37.04 |
| ATOM | 94 | CD1 | LEU | A | 49 | 70.447 | 52.989 | 34.355 | 1.00 | 37.50 |
| ATOM | 95 | CD2 | LEU | A | 49 | 72.853 | 52.643 | 35.176 | 1.00 | 37.79 |
| ATOM | 96 | N | LYS | A | 50 | 73.161 | 54.374 | 29.922 | 1.00 | 36.93 |
| ATOM | 97 | CA | LYS | A | 50 | 73.625 | 55.444 | 29.055 | 1.00 | 39.10 |
| ATOM | 98 | C | LYS | A | 50 | 73.139 | 55.299 | 27.618 | 1.00 | 40.54 |
| ATOM | 99 | O | LYS | A | 50 | 73.333 | 56.202 | 26.789 | 1.00 | 39.65 |
| ATOM | 100 | CB | LYS | A | 50 | 75.147 | 55.568 | 29.166 | 1.00 | 39.68 |
| ATOM | 101 | CG | LYS | A | 50 | 75.559 | 55.978 | 30.583 | 1.00 | 41.11 |
| ATOM | 102 | CD | LYS | A | 50 | 74.992 | 57.392 | 30.909 | 1.00 | 43.27 |
| ATOM | 103 | CE | LYS | A | 50 | 75.551 | 57.976 | 32.226 | 1.00 | 45.34 |
| ATOM | 104 | NZ | LYS | A | 50 | 75.091 | 59.395 | 32.481 | 1.00 | 44.42 |
| ATOM | 105 | N | ASN | A | 51 | 72.470 | 54.165 | 27.363 | 1.00 | 41.96 |
| ATOM | 106 | CA | ASN | A | 51 | 71.929 | 53.851 | 26.061 | 1.00 | 43.12 |
| ATOM | 107 | C | ASN | A | 51 | 73.048 | 53.801 | 25.038 | 1.00 | 43.29 |
| ATOM | 108 | O | ASN | A | 51 | 73.003 | 54.506 | 24.069 | 1.00 | 43.46 |
| ATOM | 109 | CB | ASN | A | 51 | 70.928 | 54.919 | 25.603 | 1.00 | 43.38 |
| ATOM | 110 | CG | ASN | A | 51 | 69.665 | 54.976 | 26.443 | 1.00 | 46.23 |
| ATOM | 111 | OD1 | ASN | A | 51 | 69.127 | 53.945 | 26.903 | 1.00 | 48.91 |
| ATOM | 112 | ND2 | ASN | A | 51 | 69.151 | 56.193 | 26.616 | 1.00 | 44.86 |
| ATOM | 113 | N | THR | A | 52 | 74.038 | 52.954 | 25.254 | 1.00 | 44.16 |
| ATOM | 114 | CA | THR | A | 52 | 75.150 | 52.802 | 24.336 | 1.00 | 44.31 |
| ATOM | 115 | C | THR | A | 52 | 74.698 | 52.189 | 23.020 | 1.00 | 44.61 |
| ATOM | 116 | O | THR | A | 52 | 75.284 | 52.429 | 21.971 | 1.00 | 43.95 |
| ATOM | 117 | CB | THR | A | 52 | 76.166 | 51.790 | 24.900 | 1.00 | 44.63 |
| ATOM | 118 | OG1 | THR | A | 52 | 76.595 | 52.157 | 26.200 | 1.00 | 44.54 |
| ATOM | 119 | CG2 | THR | A | 52 | 77.446 | 51.804 | 24.084 | 1.00 | 44.92 |
| ATOM | 120 | N | TYR | A | 53 | 73.707 | 51.314 | 23.125 | 1.00 | 44.37 |
| ATOM | 121 | CA | TYR | A | 53 | 73.225 | 50.540 | 22.003 | 1.00 | 43.98 |
| ATOM | 122 | C | TYR | A | 53 | 71.765 | 50.895 | 21.754 | 1.00 | 43.98 |
| ATOM | 123 | O | TYR | A | 53 | 70.856 | 50.359 | 22.395 | 1.00 | 44.20 |
| ATOM | 124 | CB | TYR | A | 53 | 73.388 | 49.068 | 22.344 | 1.00 | 43.63 |
| ATOM | 125 | CG | TYR | A | 53 | 74.835 | 48.621 | 22.567 | 1.00 | 43.01 |
| ATOM | 126 | CD1 | TYR | A | 53 | 75.744 | 48.545 | 21.521 | 1.00 | 39.95 |
| ATOM | 127 | CD2 | TYR | A | 53 | 75.277 | 48.227 | 23.840 | 1.00 | 42.07 |
| ATOM | 128 | CE1 | TYR | A | 53 | 77.071 | 48.119 | 21.740 | 1.00 | 38.67 |
| ATOM | 129 | CE2 | TYR | A | 53 | 76.574 | 47.801 | 24.062 | 1.00 | 41.06 |
| ATOM | 130 | CZ | TYR | A | 53 | 77.471 | 47.744 | 23.009 | 1.00 | 41.11 |
| ATOM | 131 | OH | TYR | A | 53 | 78.754 | 47.311 | 23.258 | 1.00 | 37.02 |
| ATOM | 132 | N | ARG | A | 54 | 71.538 | 51.831 | 20.841 | 1.00 | 44.11 |
| ATOM | 133 | CA | ARG | A | 54 | 70.188 | 52.335 | 20.571 | 1.00 | 44.03 |
| ATOM | 134 | C | ARG | A | 54 | 69.433 | 51.486 | 19.553 | 1.00 | 42.21 |
| ATOM | 135 | O | ARG | A | 54 | 70.021 | 51.014 | 18.600 | 1.00 | 41.54 |
| ATOM | 136 | CB | ARG | A | 54 | 70.279 | 53.764 | 20.036 | 1.00 | 45.08 |
| ATOM | 137 | CG | ARG | A | 54 | 70.626 | 54.779 | 21.120 | 1.00 | 51.23 |
| ATOM | 138 | CD | ARG | A | 54 | 70.507 | 56.270 | 20.718 | 1.00 | 56.63 |
| ATOM | 139 | NE | ARG | A | 54 | 71.033 | 57.116 | 21.796 | 1.00 | 61.87 |
| ATOM | 140 | CZ | ARG | A | 54 | 70.352 | 57.501 | 22.887 | 1.00 | 65.60 |
| ATOM | 141 | NH1 | ARG | A | 54 | 69.074 | 57.152 | 23.064 | 1.00 | 66.87 |
| ATOM | 142 | NH2 | ARG | A | 54 | 70.958 | 58.252 | 23.806 | 1.00 | 66.50 |
| ATOM | 143 | N | LEU | A | 55 | 68.145 | 51.285 | 19.790 | 1.00 | 40.64 |
| ATOM | 144 | CA | LEU | A | 55 | 67.256 | 50.674 | 18.818 | 1.00 | 40.22 |
| ATOM | 145 | C | LEU | A | 55 | 66.805 | 51.807 | 17.946 | 1.00 | 39.01 |
| ATOM | 146 | O | LEU | A | 55 | 66.299 | 52.781 | 18.459 | 1.00 | 39.01 |
| ATOM | 147 | CB | LEU | A | 55 | 65.976 | 50.151 | 19.461 | 1.00 | 40.01 |
| ATOM | 148 | CG | LEU | A | 55 | 65.960 | 48.891 | 20.292 | 1.00 | 40.34 |
| ATOM | 149 | CD1 | LEU | A | 55 | 64.533 | 48.667 | 20.703 | 1.00 | 42.07 |
| ATOM | 150 | CD2 | LEU | A | 55 | 66.447 | 47.705 | 19.493 | 1.00 | 40.99 |
| ATOM | 151 | N | LYS | A | 56 | 66.977 | 51.709 | 16.641 | 1.00 | 38.06 |
| ATOM | 152 | CA | LYS | A | 56 | 66.403 | 52.735 | 15.760 | 1.00 | 37.23 |
| ATOM | 153 | C | LYS | A | 56 | 64.947 | 52.390 | 15.492 | 1.00 | 35.67 |
| ATOM | 154 | O | LYS | A | 56 | 64.572 | 51.223 | 15.475 | 1.00 | 33.89 |
| ATOM | 155 | CB | LYS | A | 56 | 67.153 | 52.841 | 14.441 | 1.00 | 37.02 |
| ATOM | 156 | CG | LYS | A | 56 | 68.642 | 53.149 | 14.570 | 1.00 | 40.24 |
| ATOM | 157 | CD | LYS | A | 56 | 69.188 | 54.004 | 13.350 | 1.00 | 45.04 |
| ATOM | 158 | CE | LYS | A | 56 | 70.602 | 53.570 | 12.876 | 1.00 | 46.95 |
| ATOM | 159 | NZ | LYS | A | 56 | 70.582 | 52.395 | 11.891 | 1.00 | 48.99 |
| ATOM | 160 | N | LEU | A | 57 | 64.165 | 53.431 | 15.248 | 1.00 | 35.61 |
| ATOM | 161 | CA | LEU | A | 57 | 62.723 | 53.375 | 15.048 | 1.00 | 35.84 |
| ATOM | 162 | C | LEU | A | 57 | 62.393 | 54.023 | 13.711 | 1.00 | 34.84 |
| ATOM | 163 | O | LEU | A | 57 | 63.258 | 54.595 | 13.092 | 1.00 | 34.28 |
| ATOM | 164 | CB | LEU | A | 57 | 62.053 | 54.211 | 16.149 | 1.00 | 36.81 |
| ATOM | 165 | CG | LEU | A | 57 | 62.147 | 53.711 | 17.602 | 1.00 | 40.98 |
| ATOM | 166 | CD1 | LEU | A | 57 | 61.272 | 54.531 | 18.559 | 1.00 | 43.05 |
| ATOM | 167 | CD2 | LEU | A | 57 | 61.679 | 52.265 | 17.647 | 1.00 | 45.26 |
| ATOM | 168 | N | TYR | A | 58 | 61.132 | 53.959 | 13.294 | 1.00 | 33.45 |
| ATOM | 169 | CA | TYR | A | 58 | 60.651 | 54.643 | 12.104 | 1.00 | 32.51 |
| ATOM | 170 | C | TYR | A | 58 | 59.214 | 55.080 | 12.403 | 1.00 | 32.52 |
| ATOM | 171 | O | TYR | A | 58 | 58.252 | 54.433 | 12.024 | 1.00 | 31.59 |
| ATOM | 172 | CB | TYR | A | 58 | 60.725 | 53.744 | 10.834 | 1.00 | 31.99 |
| ATOM | 173 | CG | TYR | A | 58 | 60.721 | 54.535 | 9.547 | 1.00 | 31.12 |
| ATOM | 174 | CD1 | TYR | A | 58 | 59.532 | 55.003 | 9.017 | 1.00 | 30.51 |
| ATOM | 175 | CD2 | TYR | A | 58 | 61.920 | 54.846 | 8.867 | 1.00 | 32.94 |
| ATOM | 176 | CE1 | TYR | A | 58 | 59.498 | 55.751 | 7.824 | 1.00 | 29.91 |
| ATOM | 177 | CE2 | TYR | A | 58 | 61.905 | 55.594 | 7.651 | 1.00 | 29.33 |
| ATOM | 178 | CZ | TYR | A | 58 | 60.683 | 56.039 | 7.163 | 1.00 | 30.84 |
| ATOM | 179 | OH | TYR | A | 58 | 60.582 | 56.782 | 6.032 | 1.00 | 32.25 |
| ATOM | 180 | N | SER | A | 59 | 59.089 | 56.188 | 13.114 | 1.00 | 32.95 |
| ATOM | 181 | CA | SER | A | 59 | 57.804 | 56.732 | 13.509 | 1.00 | 32.96 |
| ATOM | 182 | C | SER | A | 59 | 57.343 | 57.664 | 12.452 | 1.00 | 32.63 |
| ATOM | 183 | O | SER | A | 59 | 57.984 | 58.673 | 12.171 | 1.00 | 33.29 |
| ATOM | 184 | CB | SER | A | 59 | 57.949 | 57.434 | 14.846 | 1.00 | 33.95 |
| ATOM | 185 | OG | SER | A | 59 | 58.527 | 56.484 | 15.747 | 1.00 | 36.22 |
| ATOM | 186 | N | LEU | A | 60 | 56.232 | 57.311 | 11.842 | 1.00 | 31.26 |
| ATOM | 187 | CA | LEU | A | 60 | 55.727 | 58.068 | 10.744 | 1.00 | 31.83 |
| ATOM | 188 | C | LEU | A | 60 | 54.307 | 58.510 | 11.018 | 1.00 | 31.39 |
| ATOM | 189 | O | LEU | A | 60 | 53.623 | 57.907 | 11.800 | 1.00 | 31.01 |
| ATOM | 190 | CB | LEU | A | 60 | 55.850 | 57.211 | 9.458 | 1.00 | 31.26 |
| ATOM | 191 | CG | LEU | A | 60 | 54.798 | 56.366 | 8.730 | 1.00 | 32.72 |
| ATOM | 192 | CD1 | LEU | A | 60 | 55.544 | 55.212 | 7.978 | 1.00 | 32.95 |
| ATOM | 193 | CD2 | LEU | A | 60 | 53.669 | 55.791 | 9.492 | 1.00 | 30.55 |
| ATOM | 194 | N | ARG | A | 61 | 53.875 | 59.568 | 10.352 | 1.00 | 32.40 |
| ATOM | 195 | CA | ARG | A | 61 | 52.511 | 60.032 | 10.491 | 1.00 | 33.55 |
| ATOM | 196 | C | ARG | A | 61 | 51.777 | 60.077 | 9.127 | 1.00 | 32.37 |
| ATOM | 197 | O | ARG | A | 61 | 52.057 | 60.942 | 8.303 | 1.00 | 31.77 |
| ATOM | 198 | CB | ARG | A | 61 | 52.524 | 61.429 | 11.117 | 1.00 | 34.89 |
| ATOM | 199 | CG | ARG | A | 61 | 53.286 | 61.606 | 12.500 | 1.00 | 39.34 |
| ATOM | 200 | CD | ARG | A | 61 | 52.946 | 62.994 | 13.190 | 1.00 | 44.62 |
| ATOM | 201 | NE | ARG | A | 61 | 53.746 | 63.318 | 14.376 | 1.00 | 50.16 |
| ATOM | 202 | CZ | ARG | A | 61 | 53.610 | 64.448 | 15.089 | 1.00 | 52.70 |
| ATOM | 203 | NH1 | ARG | A | 61 | 52.722 | 65.363 | 14.729 | 1.00 | 53.04 |
| ATOM | 204 | NH2 | ARG | A | 61 | 54.379 | 64.680 | 16.147 | 1.00 | 54.30 |
| ATOM | 205 | N | TRP | A | 62 | 50.840 | 59.171 | 8.877 | 1.00 | 31.29 |
| ATOM | 206 | CA | TRP | A | 62 | 50.101 | 59.222 | 7.613 | 1.00 | 31.61 |
| ATOM | 207 | C | TRP | A | 62 | 49.282 | 60.518 | 7.540 | 1.00 | 32.94 |
| ATOM | 208 | O | TRP | A | 62 | 48.679 | 60.886 | 8.541 | 1.00 | 33.73 |
| ATOM | 209 | CB | TRP | A | 62 | 49.159 | 58.028 | 7.468 | 1.00 | 30.78 |
| ATOM | 210 | CG | TRP | A | 62 | 49.815 | 56.694 | 7.295 | 1.00 | 28.26 |
| ATOM | 211 | CD1 | TRP | A | 62 | 49.909 | 55.690 | 8.221 | 1.00 | 28.81 |
| ATOM | 212 | CD2 | TRP | A | 62 | 50.452 | 56.191 | 6.111 | 1.00 | 26.95 |
| ATOM | 213 | NE1 | TRP | A | 62 | 50.567 | 54.600 | 7.679 | 1.00 | 26.42 |
| ATOM | 214 | CE2 | TRP | A | 62 | 50.911 | 54.887 | 6.392 | 1.00 | 21.80 |
| ATOM | 215 | CE3 | TRP | A | 62 | 50.697 | 56.721 | 4.835 | 1.00 | 27.04 |
| ATOM | 216 | CZ2 | TRP | A | 62 | 51.573 | 54.123 | 5.468 | 1.00 | 23.60 |
| ATOM | 217 | CZ3 | TRP | A | 62 | 51.353 | 55.951 | 3.924 | 1.00 | 25.98 |
| ATOM | 218 | CH2 | TRP | A | 62 | 51.804 | 54.665 | 4.251 | 1.00 | 23.38 |
| ATOM | 219 | N | ILE | A | 63 | 49.293 | 61.219 | 6.398 | 1.00 | 32.81 |
| ATOM | 220 | CA | ILE | A | 63 | 48.442 | 62.378 | 6.202 | 1.00 | 33.78 |
| ATOM | 221 | C | ILE | A | 63 | 47.425 | 62.160 | 5.121 | 1.00 | 33.73 |
| ATOM | 222 | O | ILE | A | 63 | 46.623 | 63.047 | 4.865 | 1.00 | 34.24 |
| ATOM | 223 | CB | ILE | A | 63 | 49.201 | 63.689 | 5.868 | 1.00 | 34.14 |
| ATOM | 224 | CG1 | ILE | A | 63 | 50.038 | 63.552 | 4.604 | 1.00 | 34.97 |
| ATOM | 225 | CG2 | ILE | A | 63 | 50.039 | 64.100 | 7.046 | 1.00 | 35.73 |
| ATOM | 226 | CD1 | ILE | A | 63 | 51.006 | 64.668 | 4.426 | 1.00 | 35.37 |
| ATOM | 227 | N | SER | A | 64 | 47.466 | 61.022 | 4.452 | 1.00 | 33.90 |
| ATOM | 228 | CA | SER | A | 64 | 46.484 | 60.751 | 3.424 | 1.00 | 34.57 |
| ATOM | 229 | C | SER | A | 64 | 46.523 | 59.285 | 3.113 | 1.00 | 34.76 |
| ATOM | 230 | O | SER | A | 64 | 47.091 | 58.506 | 3.861 | 1.00 | 33.73 |
| ATOM | 231 | CB | SER | A | 64 | 46.730 | 61.583 | 2.159 | 1.00 | 35.22 |
| ATOM | 232 | OG | SER | A | 64 | 47.912 | 61.164 | 1.494 | 1.00 | 36.37 |
| ATOM | 233 | N | ASP | A | 65 | 45.885 | 58.905 | 2.021 | 1.00 | 35.24 |
| ATOM | 234 | CA | ASP | A | 65 | 45.871 | 57.519 | 1.626 | 1.00 | 36.01 |
| ATOM | 235 | C | ASP | A | 65 | 47.214 | 57.090 | 1.102 | 1.00 | 34.80 |
| ATOM | 236 | O | ASP | A | 65 | 47.463 | 55.895 | 1.025 | 1.00 | 35.68 |
| ATOM | 237 | CB | ASP | A | 65 | 44.807 | 57.283 | 0.549 | 1.00 | 36.24 |
| ATOM | 238 | CG | ASP | A | 65 | 44.905 | 58.287 | −0.607 | 1.00 | 40.58 |
| ATOM | 239 | OD1 | ASP | A | 65 | 45.448 | 59.403 | −0.383 | 1.00 | 44.21 |
| ATOM | 240 | OD2 | ASP | A | 65 | 44.438 | 58.053 | −1.762 | 1.00 | 44.77 |
| ATOM | 241 | N | HIS | A | 66 | 48.065 | 58.052 | 0.756 | 1.00 | 34.26 |
| ATOM | 242 | CA | HIS | A | 66 | 49.333 | 57.704 | 0.123 | 1.00 | 34.12 |
| ATOM | 243 | C | HIS | A | 66 | 50.612 | 58.510 | 0.502 | 1.00 | 33.56 |
| ATOM | 244 | O | HIS | A | 66 | 51.687 | 58.263 | −0.053 | 1.00 | 33.66 |
| ATOM | 245 | CB | HIS | A | 66 | 49.130 | 57.697 | −1.392 | 1.00 | 34.13 |
| ATOM | 246 | CG | HIS | A | 66 | 48.904 | 59.053 | −1.966 | 1.00 | 37.13 |
| ATOM | 247 | ND1 | HIS | A | 66 | 47.755 | 59.778 | −1.723 | 1.00 | 39.90 |
| ATOM | 248 | CD2 | HIS | A | 66 | 49.681 | 59.826 | −2.760 | 1.00 | 39.35 |
| ATOM | 249 | CE1 | HIS | A | 66 | 47.842 | 60.947 | −2.337 | 1.00 | 42.55 |
| ATOM | 250 | NE2 | HIS | A | 66 | 48.999 | 61.003 | −2.975 | 1.00 | 41.85 |
| ATOM | 251 | N | GLU | A | 67 | 50.525 | 59.434 | 1.455 | 1.00 | 31.81 |
| ATOM | 252 | CA | GLU | A | 67 | 51.679 | 60.192 | 1.908 | 1.00 | 31.27 |
| ATOM | 253 | C | GLU | A | 67 | 51.826 | 60.118 | 3.419 | 1.00 | 30.54 |
| ATOM | 254 | O | GLU | A | 67 | 50.830 | 59.974 | 4.152 | 1.00 | 27.91 |
| ATOM | 255 | CB | GLU | A | 67 | 51.592 | 61.675 | 1.534 | 1.00 | 31.52 |
| ATOM | 256 | CG | GLU | A | 67 | 51.635 | 62.014 | 0.057 | 1.00 | 34.42 |
| ATOM | 257 | CD | GLU | A | 67 | 51.862 | 63.498 | −0.160 | 1.00 | 39.59 |
| ATOM | 258 | OE1 | GLU | A | 67 | 51.272 | 64.312 | 0.589 | 1.00 | 43.25 |
| ATOM | 259 | OE2 | GLU | A | 67 | 52.662 | 63.867 | −1.046 | 1.00 | 43.29 |
| ATOM | 260 | N | TYR | A | 68 | 53.078 | 60.194 | 3.882 | 1.00 | 30.78 |
| ATOM | 261 | CA | TYR | A | 68 | 53.349 | 60.283 | 5.313 | 1.00 | 31.62 |
| ATOM | 262 | C | TYR | A | 68 | 54.434 | 61.302 | 5.593 | 1.00 | 32.69 |
| ATOM | 263 | O | TYR | A | 68 | 55.267 | 61.578 | 4.717 | 1.00 | 31.58 |
| ATOM | 264 | CB | TYR | A | 68 | 53.688 | 58.928 | 5.934 | 1.00 | 31.56 |
| ATOM | 265 | CG | TYR | A | 68 | 54.984 | 58.248 | 5.506 | 1.00 | 30.01 |
| ATOM | 266 | CD1 | TYR | A | 68 | 56.212 | 58.660 | 6.002 | 1.00 | 28.17 |
| ATOM | 267 | CD2 | TYR | A | 68 | 54.964 | 57.150 | 4.625 | 1.00 | 29.70 |
| ATOM | 268 | CE1 | TYR | A | 68 | 57.411 | 58.000 | 5.635 | 1.00 | 26.63 |
| ATOM | 269 | CE2 | TYR | A | 68 | 56.142 | 56.493 | 4.245 | 1.00 | 28.44 |
| ATOM | 270 | CZ | TYR | A | 68 | 57.372 | 56.925 | 4.748 | 1.00 | 27.85 |
| ATOM | 271 | OH | TYR | A | 68 | 58.550 | 56.280 | 4.371 | 1.00 | 30.35 |
| ATOM | 272 | N | LEU | A | 69 | 54.400 | 61.860 | 6.810 | 1.00 | 33.87 |
| ATOM | 273 | CA | LEU | A | 69 | 55.400 | 62.798 | 7.294 | 1.00 | 35.36 |
| ATOM | 274 | C | LEU | A | 69 | 56.359 | 62.028 | 8.184 | 1.00 | 36.95 |
| ATOM | 275 | O | LEU | A | 69 | 55.947 | 61.095 | 8.862 | 1.00 | 36.55 |
| ATOM | 276 | CB | LEU | A | 69 | 54.767 | 63.958 | 8.060 | 1.00 | 35.63 |
| ATOM | 277 | CG | LEU | A | 69 | 53.889 | 64.916 | 7.246 | 1.00 | 35.92 |
| ATOM | 278 | CD1 | LEU | A | 69 | 53.290 | 66.024 | 8.122 | 1.00 | 37.72 |
| ATOM | 279 | CD2 | LEU | A | 69 | 54.687 | 65.522 | 6.120 | 1.00 | 37.17 |
| ATOM | 280 | N | TYR | A | 70 | 57.642 | 62.392 | 8.132 | 1.00 | 39.01 |
| ATOM | 281 | CA | TYR | A | 70 | 58.696 | 61.726 | 8.897 | 1.00 | 41.80 |
| ATOM | 282 | C | TYR | A | 70 | 59.715 | 62.785 | 9.305 | 1.00 | 44.87 |
| ATOM | 283 | O | TYR | A | 70 | 60.156 | 63.617 | 8.490 | 1.00 | 43.81 |
| ATOM | 284 | CB | TYR | A | 70 | 59.352 | 60.618 | 8.067 | 1.00 | 41.37 |
| ATOM | 285 | CG | TYR | A | 70 | 60.490 | 59.832 | 8.721 | 1.00 | 41.66 |
| ATOM | 286 | CD1 | TYR | A | 70 | 60.250 | 58.923 | 9.740 | 1.00 | 42.66 |
| ATOM | 287 | CD2 | TYR | A | 70 | 61.792 | 59.957 | 8.267 | 1.00 | 42.62 |
| ATOM | 288 | CE1 | TYR | A | 70 | 61.274 | 58.190 | 10.309 | 1.00 | 41.91 |
| ATOM | 289 | CE2 | TYR | A | 70 | 62.826 | 59.218 | 8.823 | 1.00 | 43.07 |
| ATOM | 290 | CZ | TYR | A | 70 | 62.564 | 58.351 | 9.847 | 1.00 | 43.67 |
| ATOM | 291 | OH | TYR | A | 70 | 63.594 | 57.643 | 10.399 | 1.00 | 43.42 |
| ATOM | 292 | N | LYS | A | 71 | 60.057 | 62.793 | 10.582 | 1.00 | 48.84 |
| ATOM | 293 | CA | LYS | A | 71 | 60.980 | 63.806 | 11.069 | 1.00 | 52.37 |
| ATOM | 294 | C | LYS | A | 71 | 62.343 | 63.205 | 11.258 | 1.00 | 54.20 |
| ATOM | 295 | O | LYS | A | 71 | 62.560 | 62.450 | 12.201 | 1.00 | 54.67 |
| ATOM | 296 | CB | LYS | A | 71 | 60.496 | 64.499 | 12.359 | 1.00 | 53.08 |
| ATOM | 297 | CG | LYS | A | 71 | 59.964 | 63.608 | 13.478 | 1.00 | 56.42 |
| ATOM | 298 | CD | LYS | A | 71 | 59.417 | 64.468 | 14.680 | 1.00 | 60.93 |
| ATOM | 299 | CE | LYS | A | 71 | 58.518 | 63.648 | 15.658 | 1.00 | 63.37 |
| ATOM | 300 | NZ | LYS | A | 71 | 57.109 | 63.396 | 15.156 | 1.00 | 64.08 |
| ATOM | 301 | N | GLN | A | 72 | 63.230 | 63.514 | 10.306 | 1.00 | 56.38 |
| ATOM | 302 | CA | GLN | A | 72 | 64.616 | 63.082 | 10.317 | 1.00 | 57.30 |
| ATOM | 303 | C | GLN | A | 72 | 65.450 | 64.235 | 10.843 | 1.00 | 58.19 |
| ATOM | 304 | O | GLN | A | 72 | 65.382 | 65.341 | 10.304 | 1.00 | 58.25 |
| ATOM | 305 | CB | GLN | A | 72 | 65.073 | 62.737 | 8.905 | 1.00 | 57.62 |
| ATOM | 306 | CG | GLN | A | 72 | 66.361 | 61.951 | 8.881 | 1.00 | 59.06 |
| ATOM | 307 | CD | GLN | A | 72 | 66.409 | 60.910 | 7.782 | 1.00 | 59.25 |
| ATOM | 308 | OE1 | GLN | A | 72 | 66.596 | 61.238 | 6.613 | 1.00 | 60.39 |
| ATOM | 309 | NE2 | GLN | A | 72 | 66.273 | 59.651 | 8.160 | 1.00 | 59.37 |
| ATOM | 310 | N | GLU | A | 73 | 66.258 | 63.976 | 11.872 | 1.00 | 58.85 |
| ATOM | 311 | CA | GLU | A | 73 | 67.020 | 65.043 | 12.493 | 1.00 | 59.44 |
| ATOM | 312 | C | GLU | A | 73 | 65.903 | 65.938 | 12.992 | 1.00 | 58.82 |
| ATOM | 313 | O | GLU | A | 73 | 65.064 | 65.470 | 13.791 | 1.00 | 58.69 |
| ATOM | 314 | CB | GLU | A | 73 | 67.963 | 65.718 | 11.481 | 1.00 | 59.71 |
| ATOM | 315 | CG | GLU | A | 73 | 69.086 | 64.774 | 11.062 | 1.00 | 62.37 |
| ATOM | 316 | CD | GLU | A | 73 | 69.598 | 64.988 | 9.647 | 1.00 | 64.62 |
| ATOM | 317 | OE1 | GLU | A | 73 | 69.204 | 65.973 | 8.986 | 1.00 | 66.20 |
| ATOM | 318 | OE2 | GLU | A | 73 | 70.406 | 64.154 | 9.195 | 1.00 | 66.62 |
| ATOM | 319 | N | ASN | A | 74 | 65.859 | 67.193 | 12.548 | 1.00 | 57.47 |
| ATOM | 320 | CA | ASN | A | 74 | 64.689 | 67.994 | 12.867 | 1.00 | 56.86 |
| ATOM | 321 | C | ASN | A | 74 | 63.977 | 68.548 | 11.619 | 1.00 | 54.85 |
| ATOM | 322 | O | ASN | A | 74 | 63.092 | 69.397 | 11.698 | 1.00 | 55.01 |
| ATOM | 323 | CB | ASN | A | 74 | 65.015 | 69.039 | 13.938 | 1.00 | 57.63 |
| ATOM | 324 | CG | ASN | A | 74 | 65.399 | 68.391 | 15.263 | 1.00 | 59.00 |
| ATOM | 325 | OD1 | ASN | A | 74 | 66.429 | 67.702 | 15.356 | 1.00 | 61.58 |
| ATOM | 326 | ND2 | ASN | A | 74 | 64.560 | 68.574 | 16.283 | 1.00 | 60.07 |
| ATOM | 327 | N | ASN | A | 75 | 64.330 | 68.016 | 10.462 | 1.00 | 52.29 |
| ATOM | 328 | CA | ASN | A | 75 | 63.558 | 68.319 | 9.274 | 1.00 | 50.31 |
| ATOM | 329 | C | ASN | A | 75 | 62.360 | 67.397 | 9.195 | 1.00 | 48.13 |
| ATOM | 330 | O | ASN | A | 75 | 62.425 | 66.222 | 9.570 | 1.00 | 48.00 |
| ATOM | 331 | CB | ASN | A | 75 | 64.410 | 68.186 | 8.027 | 1.00 | 50.26 |
| ATOM | 332 | CG | ASN | A | 75 | 65.573 | 69.129 | 8.049 | 1.00 | 50.23 |
| ATOM | 333 | OD1 | ASN | A | 75 | 65.446 | 70.282 | 7.691 | 1.00 | 51.42 |
| ATOM | 334 | ND2 | ASN | A | 75 | 66.697 | 68.661 | 8.542 | 1.00 | 50.94 |
| ATOM | 335 | N | ILE | A | 76 | 61.246 | 67.953 | 8.750 | 1.00 | 44.92 |
| ATOM | 336 | CA | ILE | A | 76 | 60.072 | 67.153 | 8.529 | 1.00 | 42.61 |
| ATOM | 337 | C | ILE | A | 76 | 60.024 | 66.853 | 7.052 | 1.00 | 39.17 |
| ATOM | 338 | O | ILE | A | 76 | 60.026 | 67.733 | 6.247 | 1.00 | 38.33 |
| ATOM | 339 | CB | ILE | A | 76 | 58.822 | 67.869 | 9.015 | 1.00 | 42.75 |
| ATOM | 340 | CG1 | ILE | A | 76 | 58.971 | 68.180 | 10.512 | 1.00 | 44.18 |
| ATOM | 341 | CG2 | ILE | A | 76 | 57.605 | 67.001 | 8.788 | 1.00 | 43.04 |
| ATOM | 342 | CD1 | ILE | A | 76 | 57.881 | 69.115 | 11.061 | 1.00 | 46.10 |
| ATOM | 343 | N | LEU | A | 77 | 60.002 | 65.581 | 6.731 | 1.00 | 37.03 |
| ATOM | 344 | CA | LEU | A | 77 | 60.044 | 65.121 | 5.359 | 1.00 | 35.58 |
| ATOM | 345 | C | LEU | A | 77 | 58.709 | 64.557 | 4.990 | 1.00 | 34.01 |
| ATOM | 346 | O | LEU | A | 77 | 58.071 | 63.900 | 5.786 | 1.00 | 33.20 |
| ATOM | 347 | CB | LEU | A | 77 | 61.059 | 63.991 | 5.200 | 1.00 | 34.83 |
| ATOM | 348 | CG | LEU | A | 77 | 62.442 | 64.220 | 5.769 | 1.00 | 35.71 |
| ATOM | 349 | CD1 | LEU | A | 77 | 63.355 | 63.054 | 5.417 | 1.00 | 36.23 |
| ATOM | 350 | CD2 | LEU | A | 77 | 62.997 | 65.543 | 5.208 | 1.00 | 37.79 |
| ATOM | 351 | N | VAL | A | 78 | 58.283 | 64.827 | 3.775 | 1.00 | 32.73 |
| ATOM | 352 | CA | VAL | A | 78 | 57.109 | 64.165 | 3.268 | 1.00 | 32.11 |
| ATOM | 353 | C | VAL | A | 78 | 57.565 | 63.084 | 2.282 | 1.00 | 31.94 |
| ATOM | 354 | O | VAL | A | 78 | 58.464 | 63.296 | 1.464 | 1.00 | 29.80 |
| ATOM | 355 | CB | VAL | A | 78 | 56.074 | 65.137 | 2.673 | 1.00 | 33.08 |
| ATOM | 356 | CG1 | VAL | A | 78 | 56.620 | 66.060 | 1.620 | 1.00 | 34.71 |
| ATOM | 357 | CG2 | VAL | A | 78 | 54.851 | 64.359 | 2.110 | 1.00 | 34.03 |
| ATOM | 358 | N | PHE | A | 79 | 56.976 | 61.907 | 2.459 | 1.00 | 30.10 |
| ATOM | 359 | CA | PHE | A | 79 | 57.167 | 60.723 | 1.632 | 1.00 | 30.28 |
| ATOM | 360 | C | PHE | A | 79 | 55.902 | 60.331 | 0.855 | 1.00 | 29.50 |
| ATOM | 361 | O | PHE | A | 79 | 54.796 | 60.439 | 1.369 | 1.00 | 28.83 |
| ATOM | 362 | CB | PHE | A | 79 | 57.478 | 59.542 | 2.541 | 1.00 | 30.42 |
| ATOM | 363 | CG | PHE | A | 79 | 58.882 | 59.521 | 3.032 | 1.00 | 30.64 |
| ATOM | 364 | CD1 | PHE | A | 79 | 59.339 | 60.474 | 3.937 | 1.00 | 30.69 |
| ATOM | 365 | CD2 | PHE | A | 79 | 59.753 | 58.553 | 2.591 | 1.00 | 31.60 |
| ATOM | 366 | CE1 | PHE | A | 79 | 60.651 | 60.449 | 4.378 | 1.00 | 32.56 |
| ATOM | 367 | CE2 | PHE | A | 79 | 61.078 | 58.533 | 3.040 | 1.00 | 31.97 |
| ATOM | 368 | CZ | PHE | A | 79 | 61.514 | 59.483 | 3.931 | 1.00 | 31.51 |
| ATOM | 369 | N | ASN | A | 80 | 56.095 | 59.856 | −0.370 | 1.00 | 28.48 |
| ATOM | 370 | CA | ASN | A | 80 | 55.053 | 59.271 | −1.194 | 1.00 | 28.45 |
| ATOM | 371 | C | ASN | A | 80 | 55.145 | 57.756 | −1.039 | 1.00 | 28.68 |
| ATOM | 372 | O | ASN | A | 80 | 56.177 | 57.195 | −1.298 | 1.00 | 29.34 |
| ATOM | 373 | CB | ASN | A | 80 | 55.280 | 59.664 | −2.656 | 1.00 | 28.49 |
| ATOM | 374 | CG | ASN | A | 80 | 54.274 | 59.019 | −3.593 | 1.00 | 28.68 |
| ATOM | 375 | OD1 | ASN | A | 80 | 54.264 | 57.785 | −3.747 | 1.00 | 31.70 |
| ATOM | 376 | ND2 | ASN | A | 80 | 53.440 | 59.845 | −4.238 | 1.00 | 26.72 |
| ATOM | 377 | N | ALA | A | 81 | 54.108 | 57.081 | −0.575 | 1.00 | 28.87 |
| ATOM | 378 | CA | ALA | A | 81 | 54.221 | 55.647 | −0.280 | 1.00 | 29.20 |
| ATOM | 379 | C | ALA | A | 81 | 54.367 | 54.692 | −1.486 | 1.00 | 30.47 |
| ATOM | 380 | O | ALA | A | 81 | 55.068 | 53.667 | −1.391 | 1.00 | 28.22 |
| ATOM | 381 | CB | ALA | A | 81 | 53.055 | 55.226 | 0.529 | 1.00 | 29.86 |
| ATOM | 382 | N | GLU | A | 82 | 53.692 | 55.009 | −2.584 | 1.00 | 30.69 |
| ATOM | 383 | CA | GLU | A | 82 | 53.690 | 54.163 | −3.765 | 1.00 | 32.90 |
| ATOM | 384 | C | GLU | A | 82 | 55.085 | 54.085 | −4.380 | 1.00 | 32.70 |
| ATOM | 385 | O | GLU | A | 82 | 55.584 | 53.005 | −4.642 | 1.00 | 33.55 |
| ATOM | 386 | CB | GLU | A | 82 | 52.762 | 54.778 | −4.799 | 1.00 | 34.29 |
| ATOM | 387 | CG | GLU | A | 82 | 51.904 | 53.921 | −5.750 | 1.00 | 39.11 |
| ATOM | 388 | CD | GLU | A | 82 | 51.966 | 52.395 | −5.602 | 1.00 | 44.08 |
| ATOM | 389 | OE1 | GLU | A | 82 | 51.733 | 51.877 | −4.479 | 1.00 | 44.47 |
| ATOM | 390 | OE2 | GLU | A | 82 | 52.146 | 51.715 | −6.659 | 1.00 | 45.29 |
| ATOM | 391 | N | TYR | A | 83 | 55.711 | 55.239 | −4.577 | 1.00 | 32.46 |
| ATOM | 392 | CA | TYR | A | 83 | 56.988 | 55.343 | −5.293 | 1.00 | 31.80 |
| ATOM | 393 | C | TYR | A | 83 | 58.174 | 55.575 | −4.375 | 1.00 | 32.14 |
| ATOM | 394 | O | TYR | A | 83 | 59.315 | 55.439 | −4.779 | 1.00 | 31.65 |
| ATOM | 395 | CB | TYR | A | 83 | 56.894 | 56.474 | −6.341 | 1.00 | 30.74 |
| ATOM | 396 | CG | TYR | A | 83 | 55.736 | 56.289 | −7.262 | 1.00 | 27.96 |
| ATOM | 397 | CD1 | TYR | A | 83 | 55.723 | 55.245 | −8.168 | 1.00 | 26.86 |
| ATOM | 398 | CD2 | TYR | A | 83 | 54.612 | 57.127 | −7.189 | 1.00 | 30.33 |
| ATOM | 399 | CE1 | TYR | A | 83 | 54.640 | 55.039 | −9.025 | 1.00 | 30.17 |
| ATOM | 400 | CE2 | TYR | A | 83 | 53.510 | 56.937 | −8.014 | 1.00 | 30.63 |
| ATOM | 401 | CZ | TYR | A | 83 | 53.532 | 55.881 | −8.934 | 1.00 | 32.85 |
| ATOM | 402 | OH | TYR | A | 83 | 52.481 | 55.683 | −9.777 | 1.00 | 32.80 |
| ATOM | 403 | N | GLY | A | 84 | 57.916 | 55.975 | −3.135 | 1.00 | 32.26 |
| ATOM | 404 | CA | GLY | A | 84 | 58.994 | 56.133 | −2.186 | 1.00 | 31.32 |
| ATOM | 405 | C | GLY | A | 84 | 59.847 | 57.373 | −2.335 | 1.00 | 31.68 |
| ATOM | 406 | O | GLY | A | 84 | 60.834 | 57.521 | −1.613 | 1.00 | 31.73 |
| ATOM | 407 | N | ASN | A | 85 | 59.498 | 58.278 | −3.245 | 1.00 | 31.53 |
| ATOM | 408 | CA | ASN | A | 85 | 60.243 | 59.498 | −3.337 | 1.00 | 30.97 |
| ATOM | 409 | C | ASN | A | 85 | 59.864 | 60.412 | −2.169 | 1.00 | 32.48 |
| ATOM | 410 | O | ASN | A | 85 | 58.797 | 60.259 | −1.590 | 1.00 | 31.04 |
| ATOM | 411 | CB | ASN | A | 85 | 60.048 | 60.160 | −4.684 | 1.00 | 31.28 |
| ATOM | 412 | CG | ASN | A | 85 | 58.654 | 60.659 | −4.935 | 1.00 | 30.02 |
| ATOM | 413 | OD1 | ASN | A | 85 | 57.696 | 59.886 | −5.033 | 1.00 | 28.38 |
| ATOM | 414 | ND2 | ASN | A | 85 | 58.543 | 61.981 | −5.122 | 1.00 | 30.08 |
| ATOM | 415 | N | SER | A | 86 | 60.738 | 61.346 | −1.819 | 1.00 | 33.04 |
| ATOM | 416 | CA | SER | A | 86 | 60.474 | 62.209 | −0.704 | 1.00 | 34.61 |
| ATOM | 417 | C | SER | A | 86 | 61.111 | 63.576 | −0.858 | 1.00 | 35.31 |
| ATOM | 418 | O | SER | A | 86 | 61.992 | 63.777 | −1.699 | 1.00 | 34.45 |
| ATOM | 419 | CB | SER | A | 86 | 60.946 | 61.545 | 0.578 | 1.00 | 34.40 |
| ATOM | 420 | OG | SER | A | 86 | 62.361 | 61.625 | 0.669 | 1.00 | 37.11 |
| ATOM | 421 | N | SER | A | 87 | 60.633 | 64.524 | −0.059 | 1.00 | 36.39 |
| ATOM | 422 | CA | SER | A | 87 | 61.155 | 65.892 | −0.090 | 1.00 | 37.40 |
| ATOM | 423 | C | SER | A | 87 | 61.047 | 66.508 | 1.265 | 1.00 | 38.73 |
| ATOM | 424 | O | SER | A | 87 | 60.455 | 65.924 | 2.173 | 1.00 | 37.89 |
| ATOM | 425 | CB | SER | A | 87 | 60.383 | 66.761 | −1.079 | 1.00 | 37.51 |
| ATOM | 426 | OG | SER | A | 87 | 59.126 | 66.202 | −1.378 | 1.00 | 38.91 |
| ATOM | 427 | N | VAL | A | 88 | 61.589 | 67.708 | 1.409 | 1.00 | 39.45 |
| ATOM | 428 | CA | VAL | A | 88 | 61.470 | 68.393 | 2.679 | 1.00 | 41.20 |
| ATOM | 429 | C | VAL | A | 88 | 60.213 | 69.269 | 2.720 | 1.00 | 41.11 |
| ATOM | 430 | O | VAL | A | 88 | 59.995 | 70.168 | 1.903 | 1.00 | 41.19 |
| ATOM | 431 | CB | VAL | A | 88 | 62.803 | 69.144 | 3.105 | 1.00 | 42.08 |
| ATOM | 432 | CG1 | VAL | A | 88 | 63.609 | 69.658 | 1.901 | 1.00 | 44.39 |
| ATOM | 433 | CG2 | VAL | A | 88 | 62.484 | 70.266 | 4.121 | 1.00 | 42.80 |
| ATOM | 434 | N | PHE | A | 89 | 59.354 | 68.923 | 3.668 | 1.00 | 42.02 |
| ATOM | 435 | CA | PHE | A | 89 | 58.158 | 69.691 | 3.946 | 1.00 | 42.61 |
| ATOM | 436 | C | PHE | A | 89 | 58.515 | 70.913 | 4.740 | 1.00 | 42.65 |
| ATOM | 437 | O | PHE | A | 89 | 58.174 | 72.006 | 4.372 | 1.00 | 40.97 |
| ATOM | 438 | CB | PHE | A | 89 | 57.175 | 68.901 | 4.774 | 1.00 | 43.24 |
| ATOM | 439 | CG | PHE | A | 89 | 55.908 | 69.650 | 5.053 | 1.00 | 45.25 |
| ATOM | 440 | CD1 | PHE | A | 89 | 55.192 | 70.213 | 4.019 | 1.00 | 48.71 |
| ATOM | 441 | CD2 | PHE | A | 89 | 55.450 | 69.813 | 6.342 | 1.00 | 47.88 |
| ATOM | 442 | CE1 | PHE | A | 89 | 54.028 | 70.900 | 4.266 | 1.00 | 50.50 |
| ATOM | 443 | CE2 | PHE | A | 89 | 54.292 | 70.492 | 6.594 | 1.00 | 48.14 |
| ATOM | 444 | CZ | PHE | A | 89 | 53.578 | 71.034 | 5.562 | 1.00 | 50.14 |
| ATOM | 445 | N | LEU | A | 90 | 59.219 | 70.716 | 5.843 | 1.00 | 44.35 |
| ATOM | 446 | CA | LEU | A | 90 | 59.602 | 71.839 | 6.662 | 1.00 | 46.14 |
| ATOM | 447 | C | LEU | A | 90 | 61.058 | 71.751 | 7.079 | 1.00 | 46.98 |
| ATOM | 448 | O | LEU | A | 90 | 61.460 | 70.856 | 7.826 | 1.00 | 45.46 |
| ATOM | 449 | CB | LEU | A | 90 | 58.679 | 71.910 | 7.874 | 1.00 | 46.70 |
| ATOM | 450 | CG | LEU | A | 90 | 58.692 | 73.226 | 8.634 | 1.00 | 47.52 |
| ATOM | 451 | CD1 | LEU | A | 90 | 57.850 | 73.069 | 9.878 | 1.00 | 48.98 |
| ATOM | 452 | CD2 | LEU | A | 90 | 60.091 | 73.614 | 9.015 | 1.00 | 48.42 |
| ATOM | 453 | N | GLU | A | 91 | 61.852 | 72.688 | 6.570 | 1.00 | 49.09 |
| ATOM | 454 | CA | GLU | A | 91 | 63.274 | 72.711 | 6.877 | 1.00 | 51.12 |
| ATOM | 455 | C | GLU | A | 91 | 63.458 | 72.982 | 8.338 | 1.00 | 52.09 |
| ATOM | 456 | O | GLU | A | 91 | 62.770 | 73.830 | 8.903 | 1.00 | 51.30 |
| ATOM | 457 | CB | GLU | A | 91 | 63.988 | 73.808 | 6.125 | 1.00 | 51.65 |
| ATOM | 458 | CG | GLU | A | 91 | 64.529 | 73.406 | 4.785 | 1.00 | 54.17 |
| ATOM | 459 | CD | GLU | A | 91 | 64.541 | 74.582 | 3.827 | 1.00 | 57.08 |
| ATOM | 460 | OE1 | GLU | A | 91 | 63.540 | 75.332 | 3.832 | 1.00 | 58.58 |
| ATOM | 461 | OE2 | GLU | A | 91 | 65.527 | 74.747 | 3.080 | 1.00 | 57.71 |
| ATOM | 462 | N | ASN | A | 92 | 64.419 | 72.274 | 8.920 | 1.00 | 54.09 |
| ATOM | 463 | CA | ASN | A | 92 | 64.758 | 72.390 | 10.323 | 1.00 | 55.48 |
| ATOM | 464 | C | ASN | A | 92 | 65.158 | 73.822 | 10.655 | 1.00 | 56.47 |
| ATOM | 465 | O | ASN | A | 92 | 65.072 | 74.238 | 11.798 | 1.00 | 56.53 |
| ATOM | 466 | CB | ASN | A | 92 | 65.891 | 71.404 | 10.679 | 1.00 | 56.01 |
| ATOM | 467 | CG | ASN | A | 92 | 67.215 | 71.747 | 9.995 | 1.00 | 56.92 |
| ATOM | 468 | OD1 | ASN | A | 92 | 67.374 | 72.851 | 9.475 | 1.00 | 58.85 |
| ATOM | 469 | ND2 | ASN | A | 92 | 68.167 | 70.801 | 9.994 | 1.00 | 55.37 |
| ATOM | 470 | N | SER | A | 93 | 65.580 | 74.568 | 9.641 | 1.00 | 57.67 |
| ATOM | 471 | CA | SER | A | 93 | 65.998 | 75.956 | 9.812 | 1.00 | 59.12 |
| ATOM | 472 | C | SER | A | 93 | 64.889 | 77.022 | 9.927 | 1.00 | 59.80 |
| ATOM | 473 | O | SER | A | 93 | 65.141 | 78.116 | 10.453 | 1.00 | 59.65 |
| ATOM | 474 | CB | SER | A | 93 | 66.904 | 76.360 | 8.639 | 1.00 | 59.16 |
| ATOM | 475 | OG | SER | A | 93 | 66.789 | 75.443 | 7.558 | 1.00 | 60.69 |
| ATOM | 476 | N | THR | A | 94 | 63.683 | 76.718 | 9.452 | 1.00 | 60.44 |
| ATOM | 477 | CA | THR | A | 94 | 62.608 | 77.720 | 9.370 | 1.00 | 61.40 |
| ATOM | 478 | C | THR | A | 94 | 62.407 | 78.609 | 10.610 | 1.00 | 61.76 |
| ATOM | 479 | O | THR | A | 94 | 62.315 | 79.829 | 10.506 | 1.00 | 61.08 |
| ATOM | 480 | CB | THR | A | 94 | 61.272 | 77.027 | 9.052 | 1.00 | 61.66 |
| ATOM | 481 | OG1 | THR | A | 94 | 61.411 | 76.211 | 7.881 | 1.00 | 63.17 |
| ATOM | 482 | CG2 | THR | A | 94 | 60.204 | 78.042 | 8.677 | 1.00 | 62.07 |
| ATOM | 483 | N | PHE | A | 95 | 62.318 | 77.995 | 11.781 | 1.00 | 62.61 |
| ATOM | 484 | CA | PHE | A | 95 | 62.044 | 78.753 | 12.990 | 1.00 | 63.25 |
| ATOM | 485 | C | PHE | A | 95 | 63.298 | 79.067 | 13.829 | 1.00 | 64.05 |
| ATOM | 486 | O | PHE | A | 95 | 63.249 | 79.072 | 15.050 | 1.00 | 63.10 |
| ATOM | 487 | CB | PHE | A | 95 | 60.982 | 78.015 | 13.811 | 1.00 | 63.31 |
| ATOM | 488 | CG | PHE | A | 95 | 59.767 | 77.614 | 13.009 | 1.00 | 61.93 |
| ATOM | 489 | CD1 | PHE | A | 95 | 58.862 | 78.565 | 12.582 | 1.00 | 60.41 |
| ATOM | 490 | CD2 | PHE | A | 95 | 59.536 | 76.282 | 12.687 | 1.00 | 60.39 |
| ATOM | 491 | CE1 | PHE | A | 95 | 57.746 | 78.197 | 11.854 | 1.00 | 59.51 |
| ATOM | 492 | CE2 | PHE | A | 95 | 58.433 | 75.917 | 11.967 | 1.00 | 59.98 |
| ATOM | 493 | CZ | PHE | A | 95 | 57.531 | 76.881 | 11.544 | 1.00 | 59.56 |
| ATOM | 494 | N | ASP | A | 96 | 64.418 | 79.345 | 13.163 | 1.00 | 65.57 |
| ATOM | 495 | CA | ASP | A | 96 | 65.651 | 79.720 | 13.874 | 1.00 | 66.85 |
| ATOM | 496 | C | ASP | A | 96 | 65.436 | 80.979 | 14.732 | 1.00 | 67.10 |
| ATOM | 497 | O | ASP | A | 96 | 65.902 | 81.043 | 15.863 | 1.00 | 67.29 |
| ATOM | 498 | CB | ASP | A | 96 | 66.821 | 79.952 | 12.899 | 1.00 | 67.18 |
| ATOM | 499 | CG | ASP | A | 96 | 67.442 | 78.649 | 12.383 | 1.00 | 68.19 |
| ATOM | 500 | OD1 | ASP | A | 96 | 66.948 | 77.554 | 12.742 | 1.00 | 69.92 |
| ATOM | 501 | OD2 | ASP | A | 96 | 68.432 | 78.630 | 11.613 | 1.00 | 68.66 |
| ATOM | 502 | N | GLU | A | 97 | 64.725 | 81.968 | 14.197 | 1.00 | 67.56 |
| ATOM | 503 | CA | GLU | A | 97 | 64.459 | 83.209 | 14.936 | 1.00 | 67.97 |
| ATOM | 504 | C | GLU | A | 97 | 63.127 | 83.189 | 15.714 | 1.00 | 67.64 |
| ATOM | 505 | O | GLU | A | 97 | 62.552 | 84.234 | 16.000 | 1.00 | 67.78 |
| ATOM | 506 | CB | GLU | A | 97 | 64.515 | 84.432 | 13.994 | 1.00 | 67.97 |
| ATOM | 507 | CG | GLU | A | 97 | 65.920 | 84.765 | 13.490 | 1.00 | 68.74 |
| ATOM | 508 | CD | GLU | A | 97 | 66.349 | 86.191 | 13.828 | 1.00 | 69.30 |
| ATOM | 509 | OE1 | GLU | A | 97 | 66.456 | 86.505 | 15.033 | 1.00 | 68.09 |
| ATOM | 510 | OE2 | GLU | A | 97 | 66.582 | 87.001 | 12.895 | 1.00 | 70.23 |
| ATOM | 511 | N | PHE | A | 98 | 62.648 | 82.005 | 16.066 | 1.00 | 67.34 |
| ATOM | 512 | CA | PHE | A | 98 | 61.422 | 81.898 | 16.839 | 1.00 | 67.25 |
| ATOM | 513 | C | PHE | A | 98 | 61.657 | 82.331 | 18.272 | 1.00 | 66.90 |
| ATOM | 514 | O | PHE | A | 98 | 60.790 | 82.911 | 18.906 | 1.00 | 66.31 |
| ATOM | 515 | CB | PHE | A | 98 | 60.933 | 80.464 | 16.857 | 1.00 | 67.37 |
| ATOM | 516 | CG | PHE | A | 98 | 59.548 | 80.311 | 17.401 | 1.00 | 67.61 |
| ATOM | 517 | CD1 | PHE | A | 98 | 58.468 | 80.818 | 16.715 | 1.00 | 68.05 |
| ATOM | 518 | CD2 | PHE | A | 98 | 59.325 | 79.663 | 18.597 | 1.00 | 67.98 |
| ATOM | 519 | CE1 | PHE | A | 98 | 57.190 | 80.674 | 17.211 | 1.00 | 68.39 |
| ATOM | 520 | CE2 | PHE | A | 98 | 58.046 | 79.515 | 19.092 | 1.00 | 67.80 |
| ATOM | 521 | CZ | PHE | A | 98 | 56.983 | 80.021 | 18.399 | 1.00 | 67.93 |
| ATOM | 522 | N | GLY | A | 99 | 62.833 | 82.003 | 18.788 | 1.00 | 66.78 |
| ATOM | 523 | CA | GLY | A | 99 | 63.204 | 82.374 | 20.139 | 1.00 | 66.53 |
| ATOM | 524 | C | GLY | A | 99 | 62.856 | 81.297 | 21.133 | 1.00 | 66.11 |
| ATOM | 525 | O | GLY | A | 99 | 63.455 | 81.211 | 22.201 | 1.00 | 66.86 |
| ATOM | 526 | N | HIS | A | 100 | 61.885 | 80.466 | 20.787 | 1.00 | 65.36 |
| ATOM | 527 | CA | HIS | A | 100 | 61.447 | 79.418 | 21.693 | 1.00 | 64.86 |
| ATOM | 528 | C | HIS | A | 100 | 61.783 | 78.023 | 21.161 | 1.00 | 64.00 |
| ATOM | 529 | O | HIS | A | 100 | 61.700 | 77.745 | 19.959 | 1.00 | 62.72 |
| ATOM | 530 | CB | HIS | A | 100 | 59.934 | 79.519 | 21.936 | 1.00 | 65.08 |
| ATOM | 531 | CG | HIS | A | 100 | 59.490 | 80.812 | 22.561 | 1.00 | 65.42 |
| ATOM | 532 | ND1 | HIS | A | 100 | 58.685 | 81.717 | 21.904 | 1.00 | 65.80 |
| ATOM | 533 | CD2 | HIS | A | 100 | 59.702 | 81.330 | 23.797 | 1.00 | 66.64 |
| ATOM | 534 | CE1 | HIS | A | 100 | 58.441 | 82.748 | 22.696 | 1.00 | 66.12 |
| ATOM | 535 | NE2 | HIS | A | 100 | 59.045 | 82.536 | 23.852 | 1.00 | 65.68 |
| ATOM | 536 | N | SER | A | 101 | 62.175 | 77.146 | 22.071 | 1.00 | 63.26 |
| ATOM | 537 | CA | SER | A | 101 | 62.407 | 75.760 | 21.703 | 1.00 | 62.93 |
| ATOM | 538 | C | SER | A | 101 | 61.049 | 75.092 | 21.406 | 1.00 | 62.18 |
| ATOM | 539 | O | SER | A | 101 | 60.140 | 75.073 | 22.242 | 1.00 | 61.25 |
| ATOM | 540 | CB | SER | A | 101 | 63.161 | 75.034 | 22.823 | 1.00 | 63.06 |
| ATOM | 541 | OG | SER | A | 101 | 63.767 | 73.844 | 22.335 | 1.00 | 63.28 |
| ATOM | 542 | N | ILE | A | 102 | 60.910 | 74.552 | 20.204 | 1.00 | 61.55 |
| ATOM | 543 | CA | ILE | A | 102 | 59.650 | 73.964 | 19.801 | 1.00 | 60.98 |
| ATOM | 544 | C | ILE | A | 102 | 59.558 | 72.519 | 20.239 | 1.00 | 59.90 |
| ATOM | 545 | O | ILE | A | 102 | 60.478 | 71.751 | 20.018 | 1.00 | 60.20 |
| ATOM | 546 | CB | ILE | A | 102 | 59.487 | 74.108 | 18.302 | 1.00 | 61.31 |
| ATOM | 547 | CG1 | ILE | A | 102 | 59.204 | 75.580 | 17.988 | 1.00 | 61.66 |
| ATOM | 548 | CG2 | ILE | A | 102 | 58.343 | 73.215 | 17.805 | 1.00 | 61.95 |
| ATOM | 549 | CD1 | ILE | A | 102 | 59.190 | 75.918 | 16.529 | 1.00 | 61.93 |
| ATOM | 550 | N | ASN | A | 103 | 58.446 | 72.154 | 20.873 | 1.00 | 58.45 |
| ATOM | 551 | CA | ASN | A | 103 | 58.283 | 70.808 | 21.419 | 1.00 | 57.23 |
| ATOM | 552 | C | ASN | A | 103 | 57.658 | 69.826 | 20.430 | 1.00 | 56.00 |
| ATOM | 553 | O | ASN | A | 103 | 58.051 | 68.671 | 20.332 | 1.00 | 54.77 |
| ATOM | 554 | CB | ASN | A | 103 | 57.440 | 70.863 | 22.706 | 1.00 | 57.35 |
| ATOM | 555 | CG | ASN | A | 103 | 57.481 | 69.554 | 23.496 | 1.00 | 57.35 |
| ATOM | 556 | OD1 | ASN | A | 103 | 56.598 | 68.714 | 23.352 | 1.00 | 55.48 |
| ATOM | 557 | ND2 | ASN | A | 103 | 58.507 | 69.389 | 24.348 | 1.00 | 56.22 |
| ATOM | 558 | N | ASP | A | 104 | 56.663 | 70.277 | 19.702 | 1.00 | 55.10 |
| ATOM | 559 | CA | ASP | A | 104 | 56.014 | 69.397 | 18.765 | 1.00 | 54.76 |
| ATOM | 560 | C | ASP | A | 104 | 55.324 | 70.283 | 17.769 | 1.00 | 53.89 |
| ATOM | 561 | O | ASP | A | 104 | 55.329 | 71.511 | 17.921 | 1.00 | 54.05 |
| ATOM | 562 | CB | ASP | A | 104 | 55.018 | 68.470 | 19.477 | 1.00 | 55.23 |
| ATOM | 563 | CG | ASP | A | 104 | 54.749 | 67.177 | 18.699 | 1.00 | 56.71 |
| ATOM | 564 | OD1 | ASP | A | 104 | 55.381 | 66.957 | 17.647 | 1.00 | 55.96 |
| ATOM | 565 | OD2 | ASP | A | 104 | 53.919 | 66.320 | 19.064 | 1.00 | 60.44 |
| ATOM | 566 | N | TYR | A | 105 | 54.736 | 69.656 | 16.764 | 1.00 | 52.66 |
| ATOM | 567 | CA | TYR | A | 105 | 54.086 | 70.356 | 15.687 | 1.00 | 52.56 |
| ATOM | 568 | C | TYR | A | 105 | 52.838 | 69.609 | 15.319 | 1.00 | 51.25 |
| ATOM | 569 | O | TYR | A | 105 | 52.726 | 68.403 | 15.543 | 1.00 | 51.29 |
| ATOM | 570 | CB | TYR | A | 105 | 54.976 | 70.362 | 14.448 | 1.00 | 53.10 |
| ATOM | 571 | CG | TYR | A | 105 | 55.070 | 69.000 | 13.774 | 1.00 | 55.91 |
| ATOM | 572 | CD1 | TYR | A | 105 | 54.132 | 68.603 | 12.829 | 1.00 | 59.33 |
| ATOM | 573 | CD2 | TYR | A | 105 | 56.087 | 68.106 | 14.096 | 1.00 | 58.18 |
| ATOM | 574 | CE1 | TYR | A | 105 | 54.204 | 67.347 | 12.215 | 1.00 | 60.78 |
| ATOM | 575 | CE2 | TYR | A | 105 | 56.169 | 66.855 | 13.483 | 1.00 | 59.77 |
| ATOM | 576 | CZ | TYR | A | 105 | 55.231 | 66.485 | 12.542 | 1.00 | 60.60 |
| ATOM | 577 | OH | TYR | A | 105 | 55.300 | 65.245 | 11.937 | 1.00 | 60.13 |
| ATOM | 578 | N | SER | A | 106 | 51.899 | 70.313 | 14.724 | 1.00 | 49.68 |
| ATOM | 579 | CA | SER | A | 106 | 50.719 | 69.644 | 14.246 | 1.00 | 49.10 |
| ATOM | 580 | C | SER | A | 106 | 50.252 | 70.331 | 12.986 | 1.00 | 48.51 |
| ATOM | 581 | O | SER | A | 106 | 49.835 | 71.499 | 12.976 | 1.00 | 47.69 |
| ATOM | 582 | CB | SER | A | 106 | 49.614 | 69.625 | 15.291 | 1.00 | 48.76 |
| ATOM | 583 | OG | SER | A | 106 | 48.498 | 68.968 | 14.757 | 1.00 | 48.08 |
| ATOM | 584 | N | ILE | A | 107 | 50.272 | 69.567 | 11.920 | 1.00 | 47.91 |
| ATOM | 585 | CA | ILE | A | 107 | 49.959 | 70.124 | 10.650 | 1.00 | 48.19 |
| ATOM | 586 | C | ILE | A | 107 | 48.473 | 70.005 | 10.454 | 1.00 | 48.08 |
| ATOM | 587 | O | ILE | A | 107 | 47.875 | 68.975 | 10.737 | 1.00 | 47.65 |
| ATOM | 588 | CB | ILE | A | 107 | 50.820 | 69.433 | 9.595 | 1.00 | 48.45 |
| ATOM | 589 | CG1 | ILE | A | 107 | 52.193 | 69.181 | 10.252 | 1.00 | 48.77 |
| ATOM | 590 | CG2 | ILE | A | 107 | 50.856 | 70.256 | 8.325 | 1.00 | 48.58 |
| ATOM | 591 | CD1 | ILE | A | 107 | 53.411 | 69.417 | 9.444 | 1.00 | 50.54 |
| ATOM | 592 | N | SER | A | 108 | 47.860 | 71.104 | 10.053 | 1.00 | 48.44 |
| ATOM | 593 | CA | SER | A | 108 | 46.445 | 71.086 | 9.814 | 1.00 | 49.14 |
| ATOM | 594 | C | SER | A | 108 | 46.247 | 70.064 | 8.713 | 1.00 | 49.85 |
| ATOM | 595 | O | SER | A | 108 | 47.165 | 69.818 | 7.946 | 1.00 | 50.02 |
| ATOM | 596 | CB | SER | A | 108 | 45.958 | 72.469 | 9.394 | 1.00 | 49.19 |
| ATOM | 597 | OG | SER | A | 108 | 46.787 | 73.035 | 8.386 | 1.00 | 50.05 |
| ATOM | 598 | N | PRO | A | 109 | 45.062 | 69.465 | 8.631 | 1.00 | 50.74 |
| ATOM | 599 | CA | PRO | A | 109 | 44.782 | 68.440 | 7.617 | 1.00 | 50.80 |
| ATOM | 600 | C | PRO | A | 109 | 44.722 | 68.956 | 6.185 | 1.00 | 51.14 |
| ATOM | 601 | O | PRO | A | 109 | 44.922 | 68.161 | 5.264 | 1.00 | 51.45 |
| ATOM | 602 | CB | PRO | A | 109 | 43.390 | 67.936 | 7.981 | 1.00 | 50.92 |
| ATOM | 603 | CG | PRO | A | 109 | 43.059 | 68.544 | 9.324 | 1.00 | 51.33 |
| ATOM | 604 | CD | PRO | A | 109 | 43.907 | 69.734 | 9.501 | 1.00 | 50.54 |
| ATOM | 605 | N | ASP | A | 110 | 44.391 | 70.219 | 5.965 | 1.00 | 50.90 |
| ATOM | 606 | CA | ASP | A | 110 | 44.352 | 70.690 | 4.590 | 1.00 | 51.01 |
| ATOM | 607 | C | ASP | A | 110 | 45.715 | 71.225 | 4.196 | 1.00 | 51.06 |
| ATOM | 608 | O | ASP | A | 110 | 45.851 | 71.807 | 3.139 | 1.00 | 52.40 |
| ATOM | 609 | CB | ASP | A | 110 | 43.227 | 71.703 | 4.328 | 1.00 | 50.49 |
| ATOM | 610 | CG | ASP | A | 110 | 43.344 | 72.955 | 5.182 | 1.00 | 49.53 |
| ATOM | 611 | OD1 | ASP | A | 110 | 44.363 | 73.103 | 5.860 | 1.00 | 45.06 |
| ATOM | 612 | OD2 | ASP | A | 110 | 42.466 | 73.853 | 5.223 | 1.00 | 49.22 |
| ATOM | 613 | N | GLY | A | 111 | 46.725 | 71.007 | 5.031 | 1.00 | 51.13 |
| ATOM | 614 | CA | GLY | A | 111 | 48.084 | 71.465 | 4.746 | 1.00 | 51.16 |
| ATOM | 615 | C | GLY | A | 111 | 48.338 | 72.982 | 4.684 | 1.00 | 51.30 |
| ATOM | 616 | O | GLY | A | 111 | 49.409 | 73.417 | 4.250 | 1.00 | 51.86 |
| ATOM | 617 | N | GLN | A | 112 | 47.386 | 73.797 | 5.127 | 1.00 | 50.80 |
| ATOM | 618 | CA | GLN | A | 112 | 47.535 | 75.249 | 5.078 | 1.00 | 50.53 |
| ATOM | 619 | C | GLN | A | 112 | 48.236 | 75.898 | 6.299 | 1.00 | 49.99 |
| ATOM | 620 | O | GLN | A | 112 | 48.706 | 77.038 | 6.238 | 1.00 | 49.93 |
| ATOM | 621 | CB | GLN | A | 112 | 46.149 | 75.872 | 4.878 | 1.00 | 50.71 |
| ATOM | 622 | CG | GLN | A | 112 | 45.484 | 75.478 | 3.555 | 1.00 | 51.08 |
| ATOM | 623 | CD | GLN | A | 112 | 44.189 | 76.236 | 3.297 | 1.00 | 52.53 |
| ATOM | 624 | OE1 | GLN | A | 112 | 44.104 | 77.442 | 3.556 | 1.00 | 53.75 |
| ATOM | 625 | NE2 | GLN | A | 112 | 43.179 | 75.533 | 2.798 | 1.00 | 51.77 |
| ATOM | 626 | N | PHE | A | 113 | 48.318 | 75.191 | 7.414 | 1.00 | 49.50 |
| ATOM | 627 | CA | PHE | A | 113 | 48.899 | 75.781 | 8.613 | 1.00 | 48.63 |
| ATOM | 628 | C | PHE | A | 113 | 49.547 | 74.749 | 9.488 | 1.00 | 48.52 |
| ATOM | 629 | O | PHE | A | 113 | 49.183 | 73.567 | 9.479 | 1.00 | 48.60 |
| ATOM | 630 | CB | PHE | A | 113 | 47.818 | 76.424 | 9.488 | 1.00 | 48.00 |
| ATOM | 631 | CG | PHE | A | 113 | 46.954 | 77.415 | 8.782 | 1.00 | 47.61 |
| ATOM | 632 | CD1 | PHE | A | 113 | 47.307 | 78.752 | 8.741 | 1.00 | 47.99 |
| ATOM | 633 | CD2 | PHE | A | 113 | 45.777 | 77.025 | 8.185 | 1.00 | 46.46 |
| ATOM | 634 | CE1 | PHE | A | 113 | 46.503 | 79.673 | 8.092 | 1.00 | 47.46 |
| ATOM | 635 | CE2 | PHE | A | 113 | 44.969 | 77.950 | 7.540 | 1.00 | 47.60 |
| ATOM | 636 | CZ | PHE | A | 113 | 45.333 | 79.271 | 7.491 | 1.00 | 47.63 |
| ATOM | 637 | N | ILE | A | 114 | 50.470 | 75.220 | 10.309 | 1.00 | 48.29 |
| ATOM | 638 | CA | ILE | A | 114 | 51.071 | 74.359 | 11.288 | 1.00 | 48.07 |
| ATOM | 639 | C | ILE | A | 114 | 50.970 | 74.974 | 12.683 | 1.00 | 47.45 |
| ATOM | 640 | O | ILE | A | 114 | 51.136 | 76.180 | 12.853 | 1.00 | 46.70 |
| ATOM | 641 | CB | ILE | A | 114 | 52.529 | 74.065 | 10.915 | 1.00 | 48.57 |
| ATOM | 642 | CG1 | ILE | A | 114 | 53.144 | 73.118 | 11.949 | 1.00 | 48.34 |
| ATOM | 643 | CG2 | ILE | A | 114 | 53.324 | 75.366 | 10.775 | 1.00 | 48.17 |
| ATOM | 644 | CD1 | ILE | A | 114 | 54.622 | 72.906 | 11.770 | 1.00 | 50.52 |
| ATOM | 645 | N | LEU | A | 115 | 50.670 | 74.118 | 13.660 | 1.00 | 46.94 |
| ATOM | 646 | CA | LEU | A | 115 | 50.656 | 74.482 | 15.064 | 1.00 | 46.46 |
| ATOM | 647 | C | LEU | A | 115 | 52.013 | 74.192 | 15.606 | 1.00 | 45.85 |
| ATOM | 648 | O | LEU | A | 115 | 52.494 | 73.087 | 15.461 | 1.00 | 45.54 |
| ATOM | 649 | CB | LEU | A | 115 | 49.717 | 73.572 | 15.849 | 1.00 | 46.47 |
| ATOM | 650 | CG | LEU | A | 115 | 48.381 | 74.042 | 16.381 | 1.00 | 48.15 |
| ATOM | 651 | CD1 | LEU | A | 115 | 47.816 | 72.910 | 17.237 | 1.00 | 48.28 |
| ATOM | 652 | CD2 | LEU | A | 115 | 48.504 | 75.302 | 17.189 | 1.00 | 48.34 |
| ATOM | 653 | N | LEU | A | 116 | 52.608 | 75.154 | 16.282 | 1.00 | 45.86 |
| ATOM | 654 | CA | LEU | A | 116 | 53.872 | 74.950 | 16.952 | 1.00 | 45.94 |
| ATOM | 655 | C | LEU | A | 116 | 53.597 | 74.957 | 18.447 | 1.00 | 45.74 |
| ATOM | 656 | O | LEU | A | 116 | 53.005 | 75.875 | 18.958 | 1.00 | 45.58 |
| ATOM | 657 | CB | LEU | A | 116 | 54.849 | 76.077 | 16.616 | 1.00 | 46.09 |
| ATOM | 658 | CG | LEU | A | 116 | 55.204 | 76.301 | 15.150 | 1.00 | 46.93 |
| ATOM | 659 | CD1 | LEU | A | 116 | 56.002 | 77.596 | 15.013 | 1.00 | 48.50 |
| ATOM | 660 | CD2 | LEU | A | 116 | 55.989 | 75.160 | 14.614 | 1.00 | 48.06 |
| ATOM | 661 | N | GLU | A | 117 | 54.048 | 73.936 | 19.156 | 1.00 | 46.50 |
| ATOM | 662 | CA | GLU | A | 117 | 53.799 | 73.818 | 20.587 | 1.00 | 46.10 |
| ATOM | 663 | C | GLU | A | 117 | 55.084 | 74.081 | 21.341 | 1.00 | 45.85 |
| ATOM | 664 | O | GLU | A | 117 | 56.114 | 73.520 | 21.027 | 1.00 | 46.64 |
| ATOM | 665 | CB | GLU | A | 117 | 53.295 | 72.405 | 20.871 | 1.00 | 46.38 |
| ATOM | 666 | CG | GLU | A | 117 | 53.051 | 72.060 | 22.332 | 1.00 | 46.56 |
| ATOM | 667 | CD | GLU | A | 117 | 52.655 | 70.594 | 22.516 | 1.00 | 46.13 |
| ATOM | 668 | OE1 | GLU | A | 117 | 51.560 | 70.188 | 22.081 | 1.00 | 44.34 |
| ATOM | 669 | OE2 | GLU | A | 117 | 53.434 | 69.852 | 23.130 | 1.00 | 48.23 |
| ATOM | 670 | N | TYR | A | 118 | 55.045 | 74.930 | 22.347 | 1.00 | 45.50 |
| ATOM | 671 | CA | TYR | A | 118 | 56.261 | 75.224 | 23.081 | 1.00 | 44.57 |
| ATOM | 672 | C | TYR | A | 118 | 55.894 | 75.555 | 24.530 | 1.00 | 44.74 |
| ATOM | 673 | O | TYR | A | 118 | 54.712 | 75.640 | 24.860 | 1.00 | 44.74 |
| ATOM | 674 | CB | TYR | A | 118 | 57.021 | 76.347 | 22.360 | 1.00 | 44.78 |
| ATOM | 675 | CG | TYR | A | 118 | 56.363 | 77.714 | 22.363 | 1.00 | 43.33 |
| ATOM | 676 | CD1 | TYR | A | 118 | 55.410 | 78.056 | 21.429 | 1.00 | 43.21 |
| ATOM | 677 | CD2 | TYR | A | 118 | 56.732 | 78.666 | 23.276 | 1.00 | 42.55 |
| ATOM | 678 | CE1 | TYR | A | 118 | 54.826 | 79.311 | 21.428 | 1.00 | 42.60 |
| ATOM | 679 | CE2 | TYR | A | 118 | 56.149 | 79.908 | 23.289 | 1.00 | 42.33 |
| ATOM | 680 | CZ | TYR | A | 118 | 55.200 | 80.222 | 22.362 | 1.00 | 42.64 |
| ATOM | 681 | OH | TYR | A | 118 | 54.635 | 81.462 | 22.379 | 1.00 | 43.87 |
| ATOM | 682 | N | ASN | A | 119 | 56.883 | 75.719 | 25.399 | 1.00 | 44.34 |
| ATOM | 683 | CA | ASN | A | 119 | 56.610 | 75.968 | 26.805 | 1.00 | 44.35 |
| ATOM | 684 | C | ASN | A | 119 | 55.799 | 74.775 | 27.389 | 1.00 | 43.75 |
| ATOM | 685 | O | ASN | A | 119 | 54.819 | 74.960 | 28.116 | 1.00 | 42.59 |
| ATOM | 686 | CB | ASN | A | 119 | 55.826 | 77.282 | 27.002 | 1.00 | 44.61 |
| ATOM | 687 | CG | ASN | A | 119 | 56.673 | 78.550 | 26.780 | 1.00 | 46.44 |
| ATOM | 688 | OD1 | ASN | A | 119 | 57.911 | 78.515 | 26.675 | 1.00 | 48.82 |
| ATOM | 689 | ND2 | ASN | A | 119 | 55.992 | 79.688 | 26.725 | 1.00 | 46.66 |
| ATOM | 690 | N | TYR | A | 120 | 56.207 | 73.564 | 27.031 | 1.00 | 42.78 |
| ATOM | 691 | CA | TYR | A | 120 | 55.536 | 72.357 | 27.460 | 1.00 | 42.17 |
| ATOM | 692 | C | TYR | A | 120 | 55.849 | 72.045 | 28.917 | 1.00 | 41.40 |
| ATOM | 693 | O | TYR | A | 120 | 57.000 | 71.900 | 29.320 | 1.00 | 40.43 |
| ATOM | 694 | CB | TYR | A | 120 | 55.932 | 71.195 | 26.543 | 1.00 | 43.02 |
| ATOM | 695 | CG | TYR | A | 120 | 55.770 | 69.805 | 27.131 | 1.00 | 43.77 |
| ATOM | 696 | CD1 | TYR | A | 120 | 56.670 | 69.324 | 28.058 | 1.00 | 45.93 |
| ATOM | 697 | CD2 | TYR | A | 120 | 54.715 | 68.982 | 26.757 | 1.00 | 44.47 |
| ATOM | 698 | CE1 | TYR | A | 120 | 56.542 | 68.063 | 28.592 | 1.00 | 46.89 |
| ATOM | 699 | CE2 | TYR | A | 120 | 54.573 | 67.712 | 27.295 | 1.00 | 44.14 |
| ATOM | 700 | CZ | TYR | A | 120 | 55.489 | 67.265 | 28.204 | 1.00 | 45.39 |
| ATOM | 701 | OH | TYR | A | 120 | 55.379 | 66.029 | 28.766 | 1.00 | 45.22 |
| ATOM | 702 | N | VAL | A | 121 | 54.806 | 71.982 | 29.725 | 1.00 | 40.53 |
| ATOM | 703 | CA | VAL | A | 121 | 54.974 | 71.605 | 31.121 | 1.00 | 39.35 |
| ATOM | 704 | C | VAL | A | 121 | 54.123 | 70.370 | 31.318 | 1.00 | 37.68 |
| ATOM | 705 | O | VAL | A | 121 | 52.908 | 70.408 | 31.166 | 1.00 | 37.65 |
| ATOM | 706 | CB | VAL | A | 121 | 54.554 | 72.713 | 32.051 | 1.00 | 39.90 |
| ATOM | 707 | CG1 | VAL | A | 121 | 54.784 | 72.295 | 33.502 | 1.00 | 40.45 |
| ATOM | 708 | CG2 | VAL | A | 121 | 55.362 | 73.994 | 31.720 | 1.00 | 41.03 |
| ATOM | 709 | N | LYS | A | 122 | 54.778 | 69.270 | 31.616 | 1.00 | 35.67 |
| ATOM | 710 | CA | LYS | A | 122 | 54.099 | 68.033 | 31.821 | 1.00 | 35.20 |
| ATOM | 711 | C | LYS | A | 122 | 53.217 | 68.015 | 33.063 | 1.00 | 34.14 |
| ATOM | 712 | O | LYS | A | 122 | 53.673 | 68.378 | 34.146 | 1.00 | 33.72 |
| ATOM | 713 | CB | LYS | A | 122 | 55.092 | 66.876 | 31.952 | 1.00 | 35.21 |
| ATOM | 714 | CG | LYS | A | 122 | 54.369 | 65.582 | 32.319 | 1.00 | 36.44 |
| ATOM | 715 | CD | LYS | A | 122 | 55.212 | 64.328 | 32.387 | 1.00 | 38.33 |
| ATOM | 716 | CE | LYS | A | 122 | 54.469 | 63.297 | 33.288 | 1.00 | 40.56 |
| ATOM | 717 | NZ | LYS | A | 122 | 54.953 | 61.959 | 33.075 | 1.00 | 41.12 |
| ATOM | 718 | N | GLN | A | 123 | 51.986 | 67.512 | 32.913 | 1.00 | 32.33 |
| ATOM | 719 | CA | GLN | A | 123 | 51.137 | 67.272 | 34.086 | 1.00 | 31.49 |
| ATOM | 720 | C | GLN | A | 123 | 51.141 | 65.798 | 34.419 | 1.00 | 29.73 |
| ATOM | 721 | O | GLN | A | 123 | 52.073 | 65.318 | 34.997 | 1.00 | 29.32 |
| ATOM | 722 | CB | GLN | A | 123 | 49.705 | 67.800 | 33.922 | 1.00 | 31.37 |
| ATOM | 723 | CG | GLN | A | 123 | 49.014 | 67.763 | 35.303 | 1.00 | 32.48 |
| ATOM | 724 | CD | GLN | A | 123 | 47.565 | 68.168 | 35.344 | 1.00 | 35.18 |
| ATOM | 725 | OE1 | GLN | A | 123 | 47.118 | 68.685 | 36.364 | 1.00 | 41.34 |
| ATOM | 726 | NE2 | GLN | A | 123 | 46.810 | 67.879 | 34.295 | 1.00 | 33.83 |
| ATOM | 727 | N | TRP | A | 124 | 50.113 | 65.046 | 34.049 | 1.00 | 29.19 |
| ATOM | 728 | CA | TRP | A | 124 | 50.126 | 63.619 | 34.389 | 1.00 | 28.77 |
| ATOM | 729 | C | TRP | A | 124 | 50.649 | 62.794 | 33.216 | 1.00 | 28.48 |
| ATOM | 730 | O | TRP | A | 124 | 51.496 | 63.257 | 32.505 | 1.00 | 29.03 |
| ATOM | 731 | CB | TRP | A | 124 | 48.748 | 63.166 | 34.862 | 1.00 | 28.15 |
| ATOM | 732 | CG | TRP | A | 124 | 48.171 | 64.047 | 35.916 | 1.00 | 28.17 |
| ATOM | 733 | CD1 | TRP | A | 124 | 46.971 | 64.654 | 35.882 | 1.00 | 28.36 |
| ATOM | 734 | CD2 | TRP | A | 124 | 48.767 | 64.407 | 37.168 | 1.00 | 28.10 |
| ATOM | 735 | NE1 | TRP | A | 124 | 46.763 | 65.373 | 37.026 | 1.00 | 26.42 |
| ATOM | 736 | CE2 | TRP | A | 124 | 47.846 | 65.242 | 37.841 | 1.00 | 27.48 |
| ATOM | 737 | CE3 | TRP | A | 124 | 49.988 | 64.099 | 37.798 | 1.00 | 24.94 |
| ATOM | 738 | CZ2 | TRP | A | 124 | 48.102 | 65.801 | 39.107 | 1.00 | 24.59 |
| ATOM | 739 | CZ3 | TRP | A | 124 | 50.248 | 64.652 | 39.056 | 1.00 | 26.05 |
| ATOM | 740 | CH2 | TRP | A | 124 | 49.286 | 65.477 | 39.709 | 1.00 | 24.28 |
| ATOM | 741 | N | ARG | A | 125 | 50.164 | 61.579 | 33.003 | 1.00 | 29.19 |
| ATOM | 742 | CA | ARG | A | 125 | 50.657 | 60.764 | 31.907 | 1.00 | 29.06 |
| ATOM | 743 | C | ARG | A | 125 | 50.379 | 61.345 | 30.519 | 1.00 | 29.40 |
| ATOM | 744 | O | ARG | A | 125 | 51.212 | 61.246 | 29.642 | 1.00 | 28.73 |
| ATOM | 745 | CB | ARG | A | 125 | 50.103 | 59.330 | 31.998 | 1.00 | 29.53 |
| ATOM | 746 | CG | ARG | A | 125 | 50.796 | 58.308 | 31.001 | 1.00 | 29.97 |
| ATOM | 747 | CD | ARG | A | 125 | 50.153 | 56.928 | 30.934 | 1.00 | 27.63 |
| ATOM | 748 | NE | ARG | A | 125 | 50.045 | 56.297 | 32.248 | 1.00 | 28.44 |
| ATOM | 749 | CZ | ARG | A | 125 | 50.984 | 55.535 | 32.814 | 1.00 | 30.55 |
| ATOM | 750 | NH1 | ARG | A | 125 | 52.124 | 55.282 | 32.191 | 1.00 | 29.95 |
| ATOM | 751 | NH2 | ARG | A | 125 | 50.780 | 55.005 | 34.015 | 1.00 | 29.23 |
| ATOM | 752 | N | HIS | A | 126 | 49.219 | 61.961 | 30.300 | 1.00 | 29.58 |
| ATOM | 753 | CA | HIS | A | 126 | 48.922 | 62.516 | 28.977 | 1.00 | 29.53 |
| ATOM | 754 | C | HIS | A | 126 | 48.746 | 64.039 | 28.988 | 1.00 | 29.76 |
| ATOM | 755 | O | HIS | A | 126 | 49.039 | 64.700 | 27.993 | 1.00 | 30.56 |
| ATOM | 756 | CB | HIS | A | 126 | 47.651 | 61.858 | 28.416 | 1.00 | 29.39 |
| ATOM | 757 | CG | HIS | A | 126 | 47.682 | 60.359 | 28.467 | 1.00 | 29.10 |
| ATOM | 758 | ND1 | HIS | A | 126 | 48.429 | 59.602 | 27.596 | 1.00 | 24.61 |
| ATOM | 759 | CD2 | HIS | A | 126 | 47.125 | 59.481 | 29.343 | 1.00 | 31.06 |
| ATOM | 760 | CE1 | HIS | A | 126 | 48.262 | 58.321 | 27.869 | 1.00 | 27.22 |
| ATOM | 761 | NE2 | HIS | A | 126 | 47.489 | 58.216 | 28.941 | 1.00 | 27.52 |
| ATOM | 762 | N | SER | A | 127 | 48.262 | 64.586 | 30.106 | 1.00 | 28.69 |
| ATOM | 763 | CA | SER | A | 127 | 48.038 | 66.009 | 30.240 | 1.00 | 27.65 |
| ATOM | 764 | C | SER | A | 127 | 49.338 | 66.828 | 30.359 | 1.00 | 28.78 |
| ATOM | 765 | O | SER | A | 127 | 50.381 | 66.369 | 30.886 | 1.00 | 27.69 |
| ATOM | 766 | CB | SER | A | 127 | 47.190 | 66.282 | 31.491 | 1.00 | 28.00 |
| ATOM | 767 | OG | SER | A | 127 | 47.631 | 65.524 | 32.617 | 1.00 | 23.91 |
| ATOM | 768 | N | TYR | A | 128 | 49.244 | 68.059 | 29.890 | 1.00 | 29.78 |
| ATOM | 769 | CA | TYR | A | 128 | 50.328 | 69.009 | 29.997 | 1.00 | 31.21 |
| ATOM | 770 | C | TYR | A | 128 | 49.776 | 70.328 | 29.587 | 1.00 | 31.31 |
| ATOM | 771 | O | TYR | A | 128 | 48.699 | 70.388 | 29.051 | 1.00 | 30.93 |
| ATOM | 772 | CB | TYR | A | 128 | 51.476 | 68.615 | 29.063 | 1.00 | 31.91 |
| ATOM | 773 | CG | TYR | A | 128 | 51.108 | 68.469 | 27.608 | 1.00 | 32.71 |
| ATOM | 774 | CD1 | TYR | A | 128 | 51.009 | 69.582 | 26.785 | 1.00 | 36.05 |
| ATOM | 775 | CD2 | TYR | A | 128 | 50.892 | 67.209 | 27.036 | 1.00 | 35.28 |
| ATOM | 776 | CE1 | TYR | A | 128 | 50.722 | 69.452 | 25.436 | 1.00 | 34.89 |
| ATOM | 777 | CE2 | TYR | A | 128 | 50.570 | 67.073 | 25.686 | 1.00 | 33.91 |
| ATOM | 778 | CZ | TYR | A | 128 | 50.507 | 68.190 | 24.897 | 1.00 | 34.61 |
| ATOM | 779 | OH | TYR | A | 128 | 50.201 | 68.081 | 23.563 | 1.00 | 35.38 |
| ATOM | 780 | N | THR | A | 129 | 50.471 | 71.401 | 29.901 | 1.00 | 32.61 |
| ATOM | 781 | CA | THR | A | 129 | 50.099 | 72.681 | 29.340 | 1.00 | 33.63 |
| ATOM | 782 | C | THR | A | 129 | 51.201 | 73.142 | 28.423 | 1.00 | 33.39 |
| ATOM | 783 | O | THR | A | 129 | 52.343 | 72.669 | 28.518 | 1.00 | 32.91 |
| ATOM | 784 | CB | THR | A | 129 | 49.893 | 73.739 | 30.395 | 1.00 | 34.35 |
| ATOM | 785 | OG1 | THR | A | 129 | 50.974 | 73.693 | 31.337 | 1.00 | 35.37 |
| ATOM | 786 | CG2 | THR | A | 129 | 48.609 | 73.488 | 31.192 | 1.00 | 35.67 |
| ATOM | 787 | N | ALA | A | 130 | 50.846 | 74.106 | 27.580 | 1.00 | 33.49 |
| ATOM | 788 | CA | ALA | A | 130 | 51.762 | 74.679 | 26.616 | 1.00 | 34.05 |
| ATOM | 789 | C | ALA | A | 130 | 51.226 | 75.987 | 25.993 | 1.00 | 34.58 |
| ATOM | 790 | O | ALA | A | 130 | 50.034 | 76.359 | 26.172 | 1.00 | 34.01 |
| ATOM | 791 | CB | ALA | A | 130 | 52.032 | 73.668 | 25.512 | 1.00 | 33.60 |
| ATOM | 792 | N | SER | A | 131 | 52.139 | 76.655 | 25.266 | 1.00 | 34.97 |
| ATOM | 793 | CA | SER | A | 131 | 51.879 | 77.851 | 24.480 | 1.00 | 35.90 |
| ATOM | 794 | C | SER | A | 131 | 51.829 | 77.419 | 23.032 | 1.00 | 37.17 |
| ATOM | 795 | O | SER | A | 131 | 52.506 | 76.481 | 22.657 | 1.00 | 36.47 |
| ATOM | 796 | CB | SER | A | 131 | 53.034 | 78.835 | 24.570 | 1.00 | 36.15 |
| ATOM | 797 | OG | SER | A | 131 | 53.004 | 79.607 | 25.730 | 1.00 | 35.91 |
| ATOM | 798 | N | TYR | A | 132 | 51.084 | 78.135 | 22.205 | 1.00 | 38.73 |
| ATOM | 799 | CA | TYR | A | 132 | 50.949 | 77.736 | 20.820 | 1.00 | 41.07 |
| ATOM | 800 | C | TYR | A | 132 | 51.063 | 78.887 | 19.845 | 1.00 | 42.38 |
| ATOM | 801 | O | TYR | A | 132 | 50.642 | 80.002 | 20.108 | 1.00 | 42.56 |
| ATOM | 802 | CB | TYR | A | 132 | 49.605 | 77.030 | 20.581 | 1.00 | 41.24 |
| ATOM | 803 | CG | TYR | A | 132 | 49.564 | 75.660 | 21.185 | 1.00 | 41.94 |
| ATOM | 804 | CD1 | TYR | A | 132 | 50.010 | 74.563 | 20.487 | 1.00 | 42.96 |
| ATOM | 805 | CD2 | TYR | A | 132 | 49.131 | 75.478 | 22.486 | 1.00 | 42.55 |
| ATOM | 806 | CE1 | TYR | A | 132 | 50.006 | 73.296 | 21.071 | 1.00 | 44.60 |
| ATOM | 807 | CE2 | TYR | A | 132 | 49.128 | 74.240 | 23.073 | 1.00 | 44.31 |
| ATOM | 808 | CZ | TYR | A | 132 | 49.558 | 73.148 | 22.362 | 1.00 | 43.70 |
| ATOM | 809 | OH | TYR | A | 132 | 49.550 | 71.915 | 22.958 | 1.00 | 44.24 |
| ATOM | 810 | N | ASP | A | 133 | 51.625 | 78.584 | 18.699 | 1.00 | 44.24 |
| ATOM | 811 | CA | ASP | A | 133 | 51.641 | 79.536 | 17.614 | 1.00 | 46.11 |
| ATOM | 812 | C | ASP | A | 133 | 51.122 | 78.822 | 16.398 | 1.00 | 46.98 |
| ATOM | 813 | O | ASP | A | 133 | 51.206 | 77.598 | 16.306 | 1.00 | 47.50 |
| ATOM | 814 | CB | ASP | A | 133 | 53.055 | 80.053 | 17.361 | 1.00 | 46.19 |
| ATOM | 815 | CG | ASP | A | 133 | 53.341 | 81.301 | 18.110 | 1.00 | 46.73 |
| ATOM | 816 | OD1 | ASP | A | 133 | 52.395 | 81.915 | 18.640 | 1.00 | 47.58 |
| ATOM | 817 | OD2 | ASP | A | 133 | 54.490 | 81.754 | 18.227 | 1.00 | 51.67 |
| ATOM | 818 | N | ILE | A | 134 | 50.547 | 79.573 | 15.484 | 1.00 | 48.21 |
| ATOM | 819 | CA | ILE | A | 134 | 50.161 | 78.994 | 14.243 | 1.00 | 49.72 |
| ATOM | 820 | C | ILE | A | 134 | 50.946 | 79.703 | 13.159 | 1.00 | 51.48 |
| ATOM | 821 | O | ILE | A | 134 | 50.922 | 80.919 | 13.063 | 1.00 | 51.56 |
| ATOM | 822 | CB | ILE | A | 134 | 48.702 | 79.124 | 13.999 | 1.00 | 49.74 |
| ATOM | 823 | CG1 | ILE | A | 134 | 47.917 | 78.658 | 15.216 | 1.00 | 50.32 |
| ATOM | 824 | CG2 | ILE | A | 134 | 48.344 | 78.287 | 12.797 | 1.00 | 49.73 |
| ATOM | 825 | CD1 | ILE | A | 134 | 46.477 | 79.187 | 15.260 | 1.00 | 50.42 |
| ATOM | 826 | N | TYR | A | 135 | 51.650 | 78.918 | 12.356 | 1.00 | 53.32 |
| ATOM | 827 | CA | TYR | A | 135 | 52.464 | 79.424 | 11.279 | 1.00 | 54.70 |
| ATOM | 828 | C | TYR | A | 135 | 51.735 | 79.116 | 9.964 | 1.00 | 55.69 |
| ATOM | 829 | O | TYR | A | 135 | 51.351 | 77.968 | 9.702 | 1.00 | 55.37 |
| ATOM | 830 | CB | TYR | A | 135 | 53.840 | 78.770 | 11.391 | 1.00 | 55.05 |
| ATOM | 831 | CG | TYR | A | 135 | 54.844 | 79.160 | 10.345 | 1.00 | 56.75 |
| ATOM | 832 | CD1 | TYR | A | 135 | 55.449 | 80.398 | 10.357 | 1.00 | 58.08 |
| ATOM | 833 | CD2 | TYR | A | 135 | 55.203 | 78.273 | 9.362 | 1.00 | 58.33 |
| ATOM | 834 | CE1 | TYR | A | 135 | 56.381 | 80.745 | 9.396 | 1.00 | 59.70 |
| ATOM | 835 | CE2 | TYR | A | 135 | 56.123 | 78.606 | 8.404 | 1.00 | 60.29 |
| ATOM | 836 | CZ | TYR | A | 135 | 56.714 | 79.841 | 8.424 | 1.00 | 60.03 |
| ATOM | 837 | OH | TYR | A | 135 | 57.632 | 80.157 | 7.450 | 1.00 | 61.36 |
| ATOM | 838 | N | ASP | A | 136 | 51.508 | 80.166 | 9.170 | 1.00 | 57.00 |
| ATOM | 839 | CA | ASP | A | 136 | 50.751 | 80.082 | 7.911 | 1.00 | 58.19 |
| ATOM | 840 | C | ASP | A | 136 | 51.661 | 79.653 | 6.795 | 1.00 | 59.15 |
| ATOM | 841 | O | ASP | A | 136 | 52.551 | 80.379 | 6.416 | 1.00 | 58.91 |
| ATOM | 842 | CB | ASP | A | 136 | 50.148 | 81.448 | 7.586 | 1.00 | 58.42 |
| ATOM | 843 | CG | ASP | A | 136 | 49.311 | 81.463 | 6.304 | 1.00 | 58.07 |
| ATOM | 844 | OD1 | ASP | A | 136 | 49.647 | 80.776 | 5.305 | 1.00 | 56.04 |
| ATOM | 845 | OD2 | ASP | A | 136 | 48.292 | 82.190 | 6.228 | 1.00 | 58.58 |
| ATOM | 846 | N | LEU | A | 137 | 51.386 | 78.489 | 6.233 | 1.00 | 61.13 |
| ATOM | 847 | CA | LEU | A | 137 | 52.306 | 77.855 | 5.295 | 1.00 | 62.59 |
| ATOM | 848 | C | LEU | A | 137 | 52.425 | 78.444 | 3.901 | 1.00 | 63.90 |
| ATOM | 849 | O | LEU | A | 137 | 53.532 | 78.530 | 3.382 | 1.00 | 63.88 |
| ATOM | 850 | CB | LEU | A | 137 | 51.990 | 76.368 | 5.198 | 1.00 | 62.65 |
| ATOM | 851 | CG | LEU | A | 137 | 52.645 | 75.596 | 6.341 | 1.00 | 63.06 |
| ATOM | 852 | CD1 | LEU | A | 137 | 51.922 | 74.310 | 6.631 | 1.00 | 63.27 |
| ATOM | 853 | CD2 | LEU | A | 137 | 54.088 | 75.327 | 5.981 | 1.00 | 63.98 |
| ATOM | 854 | N | ASN | A | 138 | 51.315 | 78.829 | 3.284 | 1.00 | 65.63 |
| ATOM | 855 | CA | ASN | A | 138 | 51.375 | 79.327 | 1.907 | 1.00 | 66.97 |
| ATOM | 856 | C | ASN | A | 138 | 52.144 | 80.633 | 1.857 | 1.00 | 67.75 |
| ATOM | 857 | O | ASN | A | 138 | 52.926 | 80.893 | 0.935 | 1.00 | 68.07 |
| ATOM | 858 | CB | ASN | A | 138 | 49.975 | 79.431 | 1.304 | 1.00 | 67.06 |
| ATOM | 859 | CG | ASN | A | 138 | 49.442 | 78.077 | 0.889 | 1.00 | 67.65 |
| ATOM | 860 | OD1 | ASN | A | 138 | 50.227 | 77.151 | 0.629 | 1.00 | 68.25 |
| ATOM | 861 | ND2 | ASN | A | 138 | 48.108 | 77.938 | 0.842 | 1.00 | 67.87 |
| ATOM | 862 | N | LYS | A | 139 | 51.910 | 81.448 | 2.873 | 1.00 | 68.62 |
| ATOM | 863 | CA | LYS | A | 139 | 52.738 | 82.607 | 3.119 | 1.00 | 69.62 |
| ATOM | 864 | C | LYS | A | 139 | 53.816 | 81.972 | 3.992 | 1.00 | 69.94 |
| ATOM | 865 | O | LYS | A | 139 | 53.899 | 80.750 | 4.037 | 1.00 | 70.33 |
| ATOM | 866 | CB | LYS | A | 139 | 51.935 | 83.672 | 3.855 | 1.00 | 69.82 |
| ATOM | 867 | CG | LYS | A | 139 | 50.611 | 83.997 | 3.158 | 1.00 | 70.97 |
| ATOM | 868 | CD | LYS | A | 139 | 49.587 | 84.614 | 4.104 | 1.00 | 73.07 |
| ATOM | 869 | CE | LYS | A | 139 | 48.174 | 84.576 | 3.502 | 1.00 | 74.34 |
| ATOM | 870 | NZ | LYS | A | 139 | 47.102 | 84.617 | 4.550 | 1.00 | 75.16 |
| ATOM | 871 | N | ARG | A | 140 | 54.655 | 82.754 | 4.660 | 1.00 | 70.16 |
| ATOM | 872 | CA | ARG | A | 140 | 55.630 | 82.191 | 5.602 | 1.00 | 70.26 |
| ATOM | 873 | C | ARG | A | 140 | 55.494 | 82.987 | 6.880 | 1.00 | 69.45 |
| ATOM | 874 | O | ARG | A | 140 | 56.477 | 83.343 | 7.526 | 1.00 | 69.67 |
| ATOM | 875 | CB | ARG | A | 140 | 57.044 | 82.353 | 5.077 | 1.00 | 70.82 |
| ATOM | 876 | CG | ARG | A | 140 | 57.257 | 81.894 | 3.659 | 1.00 | 73.45 |
| ATOM | 877 | CD | ARG | A | 140 | 58.665 | 82.214 | 3.142 | 1.00 | 76.89 |
| ATOM | 878 | NE | ARG | A | 140 | 58.778 | 82.051 | 1.694 | 1.00 | 79.70 |
| ATOM | 879 | CZ | ARG | A | 140 | 58.291 | 82.904 | 0.792 | 1.00 | 82.20 |
| ATOM | 880 | NH1 | ARG | A | 140 | 57.656 | 84.008 | 1.168 | 1.00 | 82.19 |
| ATOM | 881 | NH2 | ARG | A | 140 | 58.458 | 82.654 | −0.500 | 1.00 | 83.24 |
| ATOM | 882 | N | GLN | A | 141 | 54.250 | 83.240 | 7.249 | 1.00 | 68.50 |
| ATOM | 883 | CA | GLN | A | 141 | 53.934 | 84.207 | 8.282 | 1.00 | 67.79 |
| ATOM | 884 | C | GLN | A | 141 | 53.402 | 83.537 | 9.525 | 1.00 | 66.29 |
| ATOM | 885 | O | GLN | A | 141 | 52.499 | 82.716 | 9.457 | 1.00 | 65.72 |
| ATOM | 886 | CB | GLN | A | 141 | 52.851 | 85.142 | 7.718 | 1.00 | 68.09 |
| ATOM | 887 | CG | GLN | A | 141 | 52.822 | 86.583 | 8.222 | 1.00 | 69.60 |
| ATOM | 888 | CD | GLN | A | 141 | 51.681 | 87.394 | 7.570 | 1.00 | 71.88 |
| ATOM | 889 | OE1 | GLN | A | 141 | 50.694 | 87.749 | 8.226 | 1.00 | 72.03 |
| ATOM | 890 | NE2 | GLN | A | 141 | 51.811 | 87.654 | 6.273 | 1.00 | 72.19 |
| ATOM | 891 | N | LEU | A | 142 | 53.984 | 83.871 | 10.659 | 1.00 | 65.02 |
| ATOM | 892 | CA | LEU | A | 142 | 53.403 | 83.477 | 11.916 | 1.00 | 64.42 |
| ATOM | 893 | C | LEU | A | 142 | 52.126 | 84.298 | 12.008 | 1.00 | 63.42 |
| ATOM | 894 | O | LEU | A | 142 | 52.021 | 85.355 | 11.388 | 1.00 | 63.00 |
| ATOM | 895 | CB | LEU | A | 142 | 54.330 | 83.840 | 13.067 | 1.00 | 64.58 |
| ATOM | 896 | CG | LEU | A | 142 | 55.543 | 82.928 | 13.195 | 1.00 | 65.68 |
| ATOM | 897 | CD1 | LEU | A | 142 | 56.665 | 83.608 | 13.991 | 1.00 | 66.52 |
| ATOM | 898 | CD2 | LEU | A | 142 | 55.130 | 81.600 | 13.821 | 1.00 | 65.75 |
| ATOM | 899 | N | ILE | A | 143 | 51.143 | 83.830 | 12.757 | 1.00 | 62.15 |
| ATOM | 900 | CA | ILE | A | 143 | 49.930 | 84.618 | 12.900 | 1.00 | 61.48 |
| ATOM | 901 | C | ILE | A | 143 | 50.038 | 85.417 | 14.190 | 1.00 | 60.32 |
| ATOM | 902 | O | ILE | A | 143 | 50.387 | 84.867 | 15.221 | 1.00 | 60.32 |
| ATOM | 903 | CB | ILE | A | 143 | 48.690 | 83.724 | 12.870 | 1.00 | 61.54 |
| ATOM | 904 | CG1 | ILE | A | 143 | 48.543 | 83.144 | 11.468 | 1.00 | 61.17 |
| ATOM | 905 | CG2 | ILE | A | 143 | 47.454 | 84.528 | 13.238 | 1.00 | 61.79 |
| ATOM | 906 | CD1 | ILE | A | 143 | 47.407 | 82.228 | 11.299 | 1.00 | 61.35 |
| ATOM | 907 | N | THR | A | 144 | 49.773 | 86.716 | 14.115 | 1.00 | 58.81 |
| ATOM | 908 | CA | THR | A | 144 | 49.916 | 87.586 | 15.263 | 1.00 | 58.06 |
| ATOM | 909 | C | THR | A | 144 | 48.555 | 87.935 | 15.856 | 1.00 | 57.10 |
| ATOM | 910 | O | THR | A | 144 | 48.469 | 88.455 | 16.963 | 1.00 | 57.12 |
| ATOM | 911 | CB | THR | A | 144 | 50.670 | 88.874 | 14.869 | 1.00 | 58.11 |
| ATOM | 912 | OG1 | THR | A | 144 | 51.839 | 88.550 | 14.118 | 1.00 | 58.85 |
| ATOM | 913 | CG2 | THR | A | 144 | 51.246 | 89.585 | 16.105 | 1.00 | 59.32 |
| ATOM | 914 | N | GLU | A | 145 | 47.501 | 87.614 | 15.126 | 1.00 | 55.99 |
| ATOM | 915 | CA | GLU | A | 145 | 46.136 | 87.937 | 15.513 | 1.00 | 55.66 |
| ATOM | 916 | C | GLU | A | 145 | 45.459 | 86.793 | 16.258 | 1.00 | 54.33 |
| ATOM | 917 | O | GLU | A | 145 | 45.570 | 85.638 | 15.850 | 1.00 | 53.39 |
| ATOM | 918 | CB | GLU | A | 145 | 45.332 | 88.143 | 14.237 | 1.00 | 56.14 |
| ATOM | 919 | CG | GLU | A | 145 | 44.515 | 89.407 | 14.110 | 1.00 | 58.11 |
| ATOM | 920 | CD | GLU | A | 145 | 44.375 | 89.792 | 12.642 | 1.00 | 60.69 |
| ATOM | 921 | OE1 | GLU | A | 145 | 45.384 | 90.216 | 12.048 | 1.00 | 62.49 |
| ATOM | 922 | OE2 | GLU | A | 145 | 43.283 | 89.628 | 12.059 | 1.00 | 62.39 |
| ATOM | 923 | N | GLU | A | 146 | 44.733 | 87.134 | 17.321 | 1.00 | 53.21 |
| ATOM | 924 | CA | GLU | A | 146 | 43.890 | 86.192 | 18.050 | 1.00 | 52.46 |
| ATOM | 925 | C | GLU | A | 146 | 44.601 | 84.893 | 18.376 | 1.00 | 51.84 |
| ATOM | 926 | O | GLU | A | 146 | 44.125 | 83.806 | 18.042 | 1.00 | 51.80 |
| ATOM | 927 | CB | GLU | A | 146 | 42.654 | 85.904 | 17.206 | 1.00 | 52.69 |
| ATOM | 928 | CG | GLU | A | 146 | 41.898 | 87.159 | 16.814 | 1.00 | 52.58 |
| ATOM | 929 | CD | GLU | A | 146 | 41.272 | 87.854 | 18.007 | 1.00 | 52.40 |
| ATOM | 930 | OE1 | GLU | A | 146 | 41.243 | 87.256 | 19.090 | 1.00 | 51.55 |
| ATOM | 931 | OE2 | GLU | A | 146 | 40.809 | 88.995 | 17.868 | 1.00 | 53.65 |
| ATOM | 932 | N | ARG | A | 147 | 45.749 | 85.011 | 19.021 | 1.00 | 50.64 |
| ATOM | 933 | CA | ARG | A | 147 | 46.569 | 83.861 | 19.314 | 1.00 | 50.07 |
| ATOM | 934 | C | ARG | A | 147 | 45.949 | 82.956 | 20.325 | 1.00 | 48.87 |
| ATOM | 935 | O | ARG | A | 147 | 45.159 | 83.385 | 21.170 | 1.00 | 48.31 |
| ATOM | 936 | CB | ARG | A | 147 | 47.891 | 84.314 | 19.880 | 1.00 | 50.41 |
| ATOM | 937 | CG | ARG | A | 147 | 48.732 | 85.057 | 18.901 | 1.00 | 53.00 |
| ATOM | 938 | CD | ARG | A | 147 | 50.090 | 85.381 | 19.439 | 1.00 | 55.81 |
| ATOM | 939 | NE | ARG | A | 147 | 50.998 | 85.691 | 18.348 | 1.00 | 60.02 |
| ATOM | 940 | CZ | ARG | A | 147 | 52.324 | 85.592 | 18.415 | 1.00 | 64.02 |
| ATOM | 941 | NH1 | ARG | A | 147 | 52.914 | 85.181 | 19.536 | 1.00 | 63.93 |
| ATOM | 942 | NH2 | ARG | A | 147 | 53.065 | 85.902 | 17.350 | 1.00 | 66.19 |
| ATOM | 943 | N | ILE | A | 148 | 46.333 | 81.691 | 20.238 | 1.00 | 47.51 |
| ATOM | 944 | CA | ILE | A | 148 | 45.945 | 80.711 | 21.217 | 1.00 | 46.54 |
| ATOM | 945 | C | ILE | A | 148 | 46.600 | 81.226 | 22.488 | 1.00 | 45.49 |
| ATOM | 946 | O | ILE | A | 148 | 47.712 | 81.697 | 22.446 | 1.00 | 45.33 |
| ATOM | 947 | CB | ILE | A | 148 | 46.454 | 79.320 | 20.816 | 1.00 | 46.49 |
| ATOM | 948 | CG1 | ILE | A | 148 | 45.726 | 78.846 | 19.554 | 1.00 | 46.40 |
| ATOM | 949 | CG2 | ILE | A | 148 | 46.192 | 78.311 | 21.917 | 1.00 | 46.14 |
| ATOM | 950 | CD1 | ILE | A | 148 | 46.344 | 77.600 | 18.955 | 1.00 | 47.77 |
| ATOM | 951 | N | PRO | A | 149 | 45.906 | 81.209 | 23.605 | 1.00 | 44.35 |
| ATOM | 952 | CA | PRO | A | 149 | 46.501 | 81.726 | 24.840 | 1.00 | 43.90 |
| ATOM | 953 | C | PRO | A | 149 | 47.525 | 80.798 | 25.484 | 1.00 | 42.87 |
| ATOM | 954 | O | PRO | A | 149 | 47.553 | 79.610 | 25.221 | 1.00 | 41.74 |
| ATOM | 955 | CB | PRO | A | 149 | 45.324 | 81.848 | 25.799 | 1.00 | 43.71 |
| ATOM | 956 | CG | PRO | A | 149 | 44.149 | 81.190 | 25.135 | 1.00 | 45.01 |
| ATOM | 957 | CD | PRO | A | 149 | 44.537 | 80.710 | 23.783 | 1.00 | 44.57 |
| ATOM | 958 | N | ASN | A | 150 | 48.336 | 81.387 | 26.353 | 1.00 | 42.54 |
| ATOM | 959 | CA | ASN | A | 150 | 49.270 | 80.668 | 27.189 | 1.00 | 42.35 |
| ATOM | 960 | C | ASN | A | 150 | 48.520 | 79.769 | 28.137 | 1.00 | 40.60 |
| ATOM | 961 | O | ASN | A | 150 | 47.369 | 80.033 | 28.475 | 1.00 | 40.98 |
| ATOM | 962 | CB | ASN | A | 150 | 50.128 | 81.645 | 28.009 | 1.00 | 43.01 |
| ATOM | 963 | CG | ASN | A | 150 | 51.107 | 82.422 | 27.153 | 1.00 | 45.14 |
| ATOM | 964 | OD1 | ASN | A | 150 | 51.764 | 81.853 | 26.283 | 1.00 | 44.71 |
| ATOM | 965 | ND2 | ASN | A | 150 | 51.185 | 83.745 | 27.385 | 1.00 | 50.11 |
| ATOM | 966 | N | ASN | A | 151 | 49.197 | 78.715 | 28.579 | 1.00 | 39.29 |
| ATOM | 967 | CA | ASN | A | 151 | 48.637 | 77.743 | 29.508 | 1.00 | 38.12 |
| ATOM | 968 | C | ASN | A | 151 | 47.429 | 77.004 | 28.903 | 1.00 | 36.63 |
| ATOM | 969 | O | ASN | A | 151 | 46.544 | 76.538 | 29.621 | 1.00 | 34.40 |
| ATOM | 970 | CB | ASN | A | 151 | 48.284 | 78.393 | 30.858 | 1.00 | 38.43 |
| ATOM | 971 | CG | ASN | A | 151 | 49.484 | 79.088 | 31.515 | 1.00 | 40.94 |
| ATOM | 972 | OD1 | ASN | A | 151 | 50.396 | 78.429 | 32.016 | 1.00 | 47.50 |
| ATOM | 973 | ND2 | ASN | A | 151 | 49.486 | 80.411 | 31.508 | 1.00 | 39.57 |
| ATOM | 974 | N | THR | A | 152 | 47.408 | 76.889 | 27.575 | 1.00 | 35.12 |
| ATOM | 975 | CA | THR | A | 152 | 46.381 | 76.084 | 26.917 | 1.00 | 34.29 |
| ATOM | 976 | C | THR | A | 152 | 46.579 | 74.644 | 27.290 | 1.00 | 33.28 |
| ATOM | 977 | O | THR | A | 152 | 47.716 | 74.159 | 27.346 | 1.00 | 32.47 |
| ATOM | 978 | CB | THR | A | 152 | 46.433 | 76.236 | 25.428 | 1.00 | 34.54 |
| ATOM | 979 | OG1 | THR | A | 152 | 45.978 | 77.550 | 25.079 | 1.00 | 34.46 |
| ATOM | 980 | CG2 | THR | A | 152 | 45.440 | 75.341 | 24.767 | 1.00 | 34.93 |
| ATOM | 981 | N | GLN | A | 153 | 45.461 | 73.960 | 27.524 | 1.00 | 32.18 |
| ATOM | 982 | CA | GLN | A | 153 | 45.485 | 72.602 | 28.045 | 1.00 | 32.33 |
| ATOM | 983 | C | GLN | A | 153 | 45.333 | 71.526 | 26.970 | 1.00 | 32.48 |
| ATOM | 984 | O | GLN | A | 153 | 45.804 | 70.420 | 27.145 | 1.00 | 33.22 |
| ATOM | 985 | CB | GLN | A | 153 | 44.401 | 72.448 | 29.144 | 1.00 | 31.84 |
| ATOM | 986 | CG | GLN | A | 153 | 44.600 | 73.376 | 30.357 | 1.00 | 29.66 |
| ATOM | 987 | CD | GLN | A | 153 | 43.301 | 73.649 | 31.122 | 1.00 | 30.78 |
| ATOM | 988 | OE1 | GLN | A | 153 | 42.395 | 74.320 | 30.598 | 1.00 | 29.78 |
| ATOM | 989 | NE2 | GLN | A | 153 | 43.192 | 73.112 | 32.339 | 1.00 | 28.52 |
| ATOM | 990 | N | TRP | A | 154 | 44.658 | 71.838 | 25.883 | 1.00 | 32.73 |
| ATOM | 991 | CA | TRP | A | 154 | 44.553 | 70.907 | 24.769 | 1.00 | 33.07 |
| ATOM | 992 | C | TRP | A | 154 | 44.181 | 71.690 | 23.557 | 1.00 | 33.13 |
| ATOM | 993 | O | TRP | A | 154 | 43.471 | 72.646 | 23.672 | 1.00 | 32.32 |
| ATOM | 994 | CB | TRP | A | 154 | 43.483 | 69.853 | 24.982 | 1.00 | 32.74 |
| ATOM | 995 | CG | TRP | A | 154 | 43.399 | 68.894 | 23.838 | 1.00 | 34.28 |
| ATOM | 996 | CD1 | TRP | A | 154 | 42.403 | 68.807 | 22.899 | 1.00 | 36.41 |
| ATOM | 997 | CD2 | TRP | A | 154 | 44.351 | 67.895 | 23.497 | 1.00 | 34.65 |
| ATOM | 998 | NE1 | TRP | A | 154 | 42.675 | 67.806 | 22.008 | 1.00 | 35.24 |
| ATOM | 999 | CE2 | TRP | A | 154 | 43.867 | 67.222 | 22.353 | 1.00 | 36.83 |
| ATOM | 1000 | CE3 | TRP | A | 154 | 45.555 | 67.479 | 24.056 | 1.00 | 34.17 |
| ATOM | 1001 | CZ2 | TRP | A | 154 | 44.570 | 66.160 | 21.735 | 1.00 | 36.92 |
| ATOM | 1002 | CZ3 | TRP | A | 154 | 46.248 | 66.415 | 23.452 | 1.00 | 37.72 |
| ATOM | 1003 | CH2 | TRP | A | 154 | 45.752 | 65.774 | 22.298 | 1.00 | 36.01 |
| ATOM | 1004 | N | VAL | A | 155 | 44.682 | 71.264 | 22.403 | 1.00 | 34.39 |
| ATOM | 1005 | CA | VAL | A | 155 | 44.330 | 71.845 | 21.127 | 1.00 | 35.30 |
| ATOM | 1006 | C | VAL | A | 155 | 44.176 | 70.731 | 20.105 | 1.00 | 35.02 |
| ATOM | 1007 | O | VAL | A | 155 | 44.928 | 69.760 | 20.138 | 1.00 | 34.58 |
| ATOM | 1008 | CB | VAL | A | 155 | 45.443 | 72.731 | 20.562 | 1.00 | 35.77 |
| ATOM | 1009 | CG1 | VAL | A | 155 | 44.890 | 73.635 | 19.468 | 1.00 | 36.34 |
| ATOM | 1010 | CG2 | VAL | A | 155 | 46.090 | 73.548 | 21.654 | 1.00 | 38.86 |
| ATOM | 1011 | N | THR | A | 156 | 43.222 | 70.873 | 19.193 | 1.00 | 34.41 |
| ATOM | 1012 | CA | THR | A | 156 | 43.109 | 69.926 | 18.125 | 1.00 | 35.30 |
| ATOM | 1013 | C | THR | A | 156 | 42.509 | 70.539 | 16.853 | 1.00 | 35.68 |
| ATOM | 1014 | O | THR | A | 156 | 41.553 | 71.308 | 16.919 | 1.00 | 34.62 |
| ATOM | 1015 | CB | THR | A | 156 | 42.328 | 68.675 | 18.567 | 1.00 | 35.21 |
| ATOM | 1016 | OG1 | THR | A | 156 | 42.256 | 67.767 | 17.476 | 1.00 | 37.50 |
| ATOM | 1017 | CG2 | THR | A | 156 | 40.870 | 68.954 | 18.826 | 1.00 | 35.59 |
| ATOM | 1018 | N | TRP | A | 157 | 43.124 | 70.231 | 15.704 | 1.00 | 35.68 |
| ATOM | 1019 | CA | TRP | A | 157 | 42.562 | 70.616 | 14.411 | 1.00 | 36.30 |
| ATOM | 1020 | C | TRP | A | 157 | 41.330 | 69.778 | 14.186 | 1.00 | 36.28 |
| ATOM | 1021 | O | TRP | A | 157 | 41.235 | 68.679 | 14.687 | 1.00 | 35.81 |
| ATOM | 1022 | CB | TRP | A | 157 | 43.507 | 70.240 | 13.263 | 1.00 | 36.56 |
| ATOM | 1023 | CG | TRP | A | 157 | 44.754 | 71.013 | 13.176 | 1.00 | 35.29 |
| ATOM | 1024 | CD1 | TRP | A | 157 | 46.003 | 70.555 | 13.352 | 1.00 | 35.35 |
| ATOM | 1025 | CD2 | TRP | A | 157 | 44.863 | 72.388 | 12.847 | 1.00 | 33.33 |
| ATOM | 1026 | NE1 | TRP | A | 157 | 46.910 | 71.572 | 13.161 | 1.00 | 36.83 |
| ATOM | 1027 | CE2 | TRP | A | 157 | 46.224 | 72.703 | 12.817 | 1.00 | 33.51 |
| ATOM | 1028 | CE3 | TRP | A | 157 | 43.940 | 73.385 | 12.530 | 1.00 | 32.60 |
| ATOM | 1029 | CZ2 | TRP | A | 157 | 46.678 | 73.976 | 12.563 | 1.00 | 32.88 |
| ATOM | 1030 | CZ3 | TRP | A | 157 | 44.391 | 74.628 | 12.233 | 1.00 | 30.65 |
| ATOM | 1031 | CH2 | TRP | A | 157 | 45.741 | 74.927 | 12.265 | 1.00 | 30.48 |
| ATOM | 1032 | N | SER | A | 158 | 40.407 | 70.290 | 13.404 | 1.00 | 37.62 |
| ATOM | 1033 | CA | SER | A | 158 | 39.260 | 69.503 | 12.980 | 1.00 | 38.44 |
| ATOM | 1034 | C | SER | A | 158 | 39.858 | 68.436 | 12.063 | 1.00 | 39.22 |
| ATOM | 1035 | O | SER | A | 158 | 41.005 | 68.554 | 11.675 | 1.00 | 38.90 |
| ATOM | 1036 | CB | SER | A | 158 | 38.261 | 70.381 | 12.251 | 1.00 | 37.53 |
| ATOM | 1037 | OG | SER | A | 158 | 38.943 | 71.285 | 11.441 | 1.00 | 36.37 |
| ATOM | 1038 | N | PRO | A | 159 | 39.127 | 67.384 | 11.736 | 1.00 | 40.86 |
| ATOM | 1039 | CA | PRO | A | 159 | 39.745 | 66.256 | 11.014 | 1.00 | 42.66 |
| ATOM | 1040 | C | PRO | A | 159 | 40.010 | 66.532 | 9.552 | 1.00 | 44.36 |
| ATOM | 1041 | O | PRO | A | 159 | 40.795 | 65.810 | 8.942 | 1.00 | 45.26 |
| ATOM | 1042 | CB | PRO | A | 159 | 38.708 | 65.138 | 11.103 | 1.00 | 42.08 |
| ATOM | 1043 | CG | PRO | A | 159 | 37.607 | 65.652 | 11.896 | 1.00 | 42.11 |
| ATOM | 1044 | CD | PRO | A | 159 | 37.709 | 67.164 | 11.985 | 1.00 | 40.54 |
| ATOM | 1045 | N | VAL | A | 160 | 39.383 | 67.558 | 9.004 | 1.00 | 46.04 |
| ATOM | 1046 | CA | VAL | A | 160 | 39.487 | 67.800 | 7.592 | 1.00 | 47.33 |
| ATOM | 1047 | C | VAL | A | 160 | 39.335 | 69.255 | 7.251 | 1.00 | 47.02 |
| ATOM | 1048 | O | VAL | A | 160 | 38.227 | 69.687 | 6.981 | 1.00 | 48.15 |
| ATOM | 1049 | CB | VAL | A | 160 | 38.351 | 67.046 | 6.891 | 1.00 | 48.20 |
| ATOM | 1050 | CG1 | VAL | A | 160 | 37.857 | 67.793 | 5.706 | 1.00 | 48.33 |
| ATOM | 1051 | CG2 | VAL | A | 160 | 38.803 | 65.639 | 6.499 | 1.00 | 49.68 |
| ATOM | 1052 | N | GLY | A | 161 | 40.428 | 70.010 | 7.237 | 1.00 | 45.77 |
| ATOM | 1053 | CA | GLY | A | 161 | 40.341 | 71.439 | 6.972 | 1.00 | 44.86 |
| ATOM | 1054 | C | GLY | A | 161 | 41.305 | 72.178 | 7.886 | 1.00 | 43.51 |
| ATOM | 1055 | O | GLY | A | 161 | 42.466 | 71.822 | 7.964 | 1.00 | 42.53 |
| ATOM | 1056 | N | HIS | A | 162 | 40.850 | 73.220 | 8.566 | 1.00 | 42.39 |
| ATOM | 1057 | CA | HIS | A | 162 | 41.748 | 73.892 | 9.512 | 1.00 | 41.52 |
| ATOM | 1058 | C | HIS | A | 162 | 41.047 | 74.636 | 10.666 | 1.00 | 40.24 |
| ATOM | 1059 | O | HIS | A | 162 | 41.517 | 75.654 | 11.123 | 1.00 | 38.94 |
| ATOM | 1060 | CB | HIS | A | 162 | 42.699 | 74.805 | 8.752 | 1.00 | 41.07 |
| ATOM | 1061 | CG | HIS | A | 162 | 42.007 | 75.869 | 7.976 | 1.00 | 42.04 |
| ATOM | 1062 | ND1 | HIS | A | 162 | 42.150 | 76.002 | 6.611 | 1.00 | 43.01 |
| ATOM | 1063 | CD2 | HIS | A | 162 | 41.174 | 76.861 | 8.371 | 1.00 | 42.61 |
| ATOM | 1064 | CE1 | HIS | A | 162 | 41.416 | 77.022 | 6.199 | 1.00 | 44.93 |
| ATOM | 1065 | NE2 | HIS | A | 162 | 40.813 | 77.559 | 7.248 | 1.00 | 43.01 |
| ATOM | 1066 | N | LYS | A | 163 | 39.900 | 74.139 | 11.101 | 1.00 | 40.02 |
| ATOM | 1067 | CA | LYS | A | 163 | 39.255 | 74.668 | 12.297 | 1.00 | 39.80 |
| ATOM | 1068 | C | LYS | A | 163 | 40.092 | 74.243 | 13.507 | 1.00 | 39.23 |
| ATOM | 1069 | O | LYS | A | 163 | 40.831 | 73.277 | 13.436 | 1.00 | 39.27 |
| ATOM | 1070 | CB | LYS | A | 163 | 37.825 | 74.133 | 12.427 | 1.00 | 40.08 |
| ATOM | 1071 | CG | LYS | A | 163 | 36.841 | 74.602 | 11.337 | 1.00 | 41.67 |
| ATOM | 1072 | CD | LYS | A | 163 | 35.389 | 74.250 | 11.717 | 1.00 | 43.49 |
| ATOM | 1073 | CE | LYS | A | 163 | 34.377 | 74.600 | 10.604 | 1.00 | 45.24 |
| ATOM | 1074 | NZ | LYS | A | 163 | 33.021 | 74.073 | 10.922 | 1.00 | 44.53 |
| ATOM | 1075 | N | LEU | A | 164 | 40.002 | 74.984 | 14.612 | 1.00 | 38.88 |
| ATOM | 1076 | CA | LEU | A | 164 | 40.754 | 74.659 | 15.817 | 1.00 | 38.35 |
| ATOM | 1077 | C | LEU | A | 164 | 39.831 | 74.614 | 17.021 | 1.00 | 37.44 |
| ATOM | 1078 | O | LEU | A | 164 | 38.947 | 75.453 | 17.144 | 1.00 | 37.74 |
| ATOM | 1079 | CB | LEU | A | 164 | 41.819 | 75.723 | 16.098 | 1.00 | 38.44 |
| ATOM | 1080 | CG | LEU | A | 164 | 43.153 | 75.629 | 15.373 | 1.00 | 39.70 |
| ATOM | 1081 | CD1 | LEU | A | 164 | 43.971 | 76.881 | 15.604 | 1.00 | 40.58 |
| ATOM | 1082 | CD2 | LEU | A | 164 | 43.939 | 74.433 | 15.817 | 1.00 | 39.25 |
| ATOM | 1083 | N | ALA | A | 165 | 40.040 | 73.641 | 17.909 | 1.00 | 36.10 |
| ATOM | 1084 | CA | ALA | A | 165 | 39.347 | 73.630 | 19.184 | 1.00 | 35.22 |
| ATOM | 1085 | C | ALA | A | 165 | 40.381 | 73.531 | 20.277 | 1.00 | 34.55 |
| ATOM | 1086 | O | ALA | A | 165 | 41.298 | 72.755 | 20.204 | 1.00 | 33.31 |
| ATOM | 1087 | CB | ALA | A | 165 | 38.310 | 72.508 | 19.289 | 1.00 | 34.85 |
| ATOM | 1088 | N | TYR | A | 166 | 40.234 | 74.337 | 21.311 | 1.00 | 34.62 |
| ATOM | 1089 | CA | TYR | A | 166 | 41.186 | 74.283 | 22.397 | 1.00 | 33.94 |
| ATOM | 1090 | C | TYR | A | 166 | 40.522 | 74.510 | 23.727 | 1.00 | 33.50 |
| ATOM | 1091 | O | TYR | A | 166 | 39.417 | 75.045 | 23.809 | 1.00 | 31.59 |
| ATOM | 1092 | CB | TYR | A | 166 | 42.299 | 75.291 | 22.179 | 1.00 | 34.14 |
| ATOM | 1093 | CG | TYR | A | 166 | 41.868 | 76.728 | 22.206 | 1.00 | 36.02 |
| ATOM | 1094 | CD1 | TYR | A | 166 | 41.811 | 77.419 | 23.410 | 1.00 | 38.52 |
| ATOM | 1095 | CD2 | TYR | A | 166 | 41.585 | 77.417 | 21.045 | 1.00 | 38.10 |
| ATOM | 1096 | CE1 | TYR | A | 166 | 41.470 | 78.729 | 23.473 | 1.00 | 38.65 |
| ATOM | 1097 | CE2 | TYR | A | 166 | 41.245 | 78.775 | 21.097 | 1.00 | 39.86 |
| ATOM | 1098 | CZ | TYR | A | 166 | 41.184 | 79.413 | 22.335 | 1.00 | 38.33 |
| ATOM | 1099 | OH | TYR | A | 166 | 40.824 | 80.725 | 22.463 | 1.00 | 35.71 |
| ATOM | 1100 | N | VAL | A | 167 | 41.248 | 74.105 | 24.767 | 1.00 | 33.32 |
| ATOM | 1101 | CA | VAL | A | 167 | 40.771 | 74.134 | 26.123 | 1.00 | 33.62 |
| ATOM | 1102 | C | VAL | A | 167 | 41.739 | 74.941 | 26.943 | 1.00 | 34.02 |
| ATOM | 1103 | O | VAL | A | 167 | 42.924 | 74.627 | 26.996 | 1.00 | 33.50 |
| ATOM | 1104 | CB | VAL | A | 167 | 40.723 | 72.732 | 26.689 | 1.00 | 33.48 |
| ATOM | 1105 | CG1 | VAL | A | 167 | 40.393 | 72.763 | 28.202 | 1.00 | 33.98 |
| ATOM | 1106 | CG2 | VAL | A | 167 | 39.737 | 71.916 | 25.934 | 1.00 | 34.69 |
| ATOM | 1107 | N | TRP | A | 168 | 41.212 | 75.977 | 27.580 | 1.00 | 34.37 |
| ATOM | 1108 | CA | TRP | A | 168 | 41.988 | 76.887 | 28.405 | 1.00 | 35.04 |
| ATOM | 1109 | C | TRP | A | 168 | 41.126 | 77.196 | 29.624 | 1.00 | 34.81 |
| ATOM | 1110 | O | TRP | A | 168 | 39.932 | 77.500 | 29.487 | 1.00 | 34.71 |
| ATOM | 1111 | CB | TRP | A | 168 | 42.292 | 78.150 | 27.603 | 1.00 | 35.59 |
| ATOM | 1112 | CG | TRP | A | 168 | 43.055 | 79.241 | 28.346 | 1.00 | 37.17 |
| ATOM | 1113 | CD1 | TRP | A | 168 | 44.354 | 79.214 | 28.720 | 1.00 | 37.65 |
| ATOM | 1114 | CD2 | TRP | A | 168 | 42.547 | 80.506 | 28.752 | 1.00 | 38.52 |
| ATOM | 1115 | NE1 | TRP | A | 168 | 44.695 | 80.380 | 29.353 | 1.00 | 39.17 |
| ATOM | 1116 | CE2 | TRP | A | 168 | 43.596 | 81.195 | 29.387 | 1.00 | 41.72 |
| ATOM | 1117 | CE3 | TRP | A | 168 | 41.310 | 81.130 | 28.647 | 1.00 | 41.50 |
| ATOM | 1118 | CZ2 | TRP | A | 168 | 43.444 | 82.489 | 29.912 | 1.00 | 43.72 |
| ATOM | 1119 | CZ3 | TRP | A | 168 | 41.152 | 82.414 | 29.179 | 1.00 | 44.45 |
| ATOM | 1120 | CH2 | TRP | A | 168 | 42.213 | 83.073 | 29.796 | 1.00 | 43.32 |
| ATOM | 1121 | N | ASN | A | 169 | 41.711 | 77.079 | 30.811 | 1.00 | 34.34 |
| ATOM | 1122 | CA | ASN | A | 169 | 40.989 | 77.316 | 32.045 | 1.00 | 34.21 |
| ATOM | 1123 | C | ASN | A | 169 | 39.729 | 76.453 | 32.119 | 1.00 | 33.09 |
| ATOM | 1124 | O | ASN | A | 169 | 38.691 | 76.851 | 32.618 | 1.00 | 30.84 |
| ATOM | 1125 | CB | ASN | A | 169 | 40.688 | 78.805 | 32.210 | 1.00 | 34.41 |
| ATOM | 1126 | CG | ASN | A | 169 | 41.888 | 79.576 | 32.756 | 1.00 | 38.60 |
| ATOM | 1127 | OD1 | ASN | A | 169 | 41.801 | 80.778 | 33.014 | 1.00 | 44.54 |
| ATOM | 1128 | ND2 | ASN | A | 169 | 43.012 | 78.882 | 32.954 | 1.00 | 39.87 |
| ATOM | 1129 | N | ASN | A | 170 | 39.862 | 75.244 | 31.596 | 1.00 | 32.83 |
| ATOM | 1130 | CA | ASN | A | 170 | 38.842 | 74.219 | 31.682 | 1.00 | 32.10 |
| ATOM | 1131 | C | ASN | A | 170 | 37.615 | 74.477 | 30.844 | 1.00 | 31.06 |
| ATOM | 1132 | O | ASN | A | 170 | 36.624 | 73.782 | 31.001 | 1.00 | 30.55 |
| ATOM | 1133 | CB | ASN | A | 170 | 38.462 | 73.971 | 33.153 | 1.00 | 32.85 |
| ATOM | 1134 | CG | ASN | A | 170 | 39.577 | 73.286 | 33.945 | 1.00 | 33.35 |
| ATOM | 1135 | OD1 | ASN | A | 170 | 40.751 | 73.469 | 33.679 | 1.00 | 34.79 |
| ATOM | 1136 | ND2 | ASN | A | 170 | 39.192 | 72.515 | 34.937 | 1.00 | 32.85 |
| ATOM | 1137 | N | ASP | A | 171 | 37.664 | 75.460 | 29.948 | 1.00 | 31.17 |
| ATOM | 1138 | CA | ASP | A | 171 | 36.562 | 75.684 | 29.005 | 1.00 | 31.06 |
| ATOM | 1139 | C | ASP | A | 171 | 37.013 | 75.482 | 27.535 | 1.00 | 30.59 |
| ATOM | 1140 | O | ASP | A | 171 | 38.167 | 75.659 | 27.190 | 1.00 | 30.25 |
| ATOM | 1141 | CB | ASP | A | 171 | 35.993 | 77.097 | 29.148 | 1.00 | 31.19 |
| ATOM | 1142 | CG | ASP | A | 171 | 35.138 | 77.270 | 30.383 | 1.00 | 31.15 |
| ATOM | 1143 | OD1 | ASP | A | 171 | 34.224 | 76.431 | 30.664 | 1.00 | 29.15 |
| ATOM | 1144 | OD2 | ASP | A | 171 | 35.321 | 78.238 | 31.125 | 1.00 | 30.82 |
| ATOM | 1145 | N | ILE | A | 172 | 36.067 | 75.147 | 26.673 | 1.00 | 30.17 |
| ATOM | 1146 | CA | ILE | A | 172 | 36.339 | 74.932 | 25.264 | 1.00 | 30.16 |
| ATOM | 1147 | C | ILE | A | 172 | 36.173 | 76.191 | 24.444 | 1.00 | 30.54 |
| ATOM | 1148 | O | ILE | A | 172 | 35.215 | 76.910 | 24.627 | 1.00 | 30.37 |
| ATOM | 1149 | CB | ILE | A | 172 | 35.385 | 73.882 | 24.724 | 1.00 | 29.51 |
| ATOM | 1150 | CG1 | ILE | A | 172 | 35.615 | 72.570 | 25.450 | 1.00 | 27.93 |
| ATOM | 1151 | CG2 | ILE | A | 172 | 35.583 | 73.729 | 23.235 | 1.00 | 30.53 |
| ATOM | 1152 | CD1 | ILE | A | 172 | 34.434 | 71.624 | 25.459 | 1.00 | 29.06 |
| ATOM | 1153 | N | TYR | A | 173 | 37.127 | 76.456 | 23.563 | 1.00 | 31.38 |
| ATOM | 1154 | CA | TYR | A | 173 | 37.010 | 77.526 | 22.590 | 1.00 | 33.28 |
| ATOM | 1155 | C | TYR | A | 173 | 37.193 | 76.928 | 21.181 | 1.00 | 34.60 |
| ATOM | 1156 | O | TYR | A | 173 | 37.876 | 75.901 | 21.016 | 1.00 | 33.51 |
| ATOM | 1157 | CB | TYR | A | 173 | 38.037 | 78.633 | 22.833 | 1.00 | 33.55 |
| ATOM | 1158 | CG | TYR | A | 173 | 37.867 | 79.289 | 24.189 | 1.00 | 33.93 |
| ATOM | 1159 | CD1 | TYR | A | 173 | 38.130 | 78.577 | 25.344 | 1.00 | 35.75 |
| ATOM | 1160 | CD2 | TYR | A | 173 | 37.415 | 80.602 | 24.311 | 1.00 | 32.65 |
| ATOM | 1161 | CE1 | TYR | A | 173 | 37.972 | 79.160 | 26.609 | 1.00 | 37.59 |
| ATOM | 1162 | CE2 | TYR | A | 173 | 37.236 | 81.198 | 25.571 | 1.00 | 34.72 |
| ATOM | 1163 | CZ | TYR | A | 173 | 37.524 | 80.474 | 26.711 | 1.00 | 36.29 |
| ATOM | 1164 | OH | TYR | A | 173 | 37.352 | 81.009 | 27.965 | 1.00 | 36.67 |
| ATOM | 1165 | N | VAL | A | 174 | 36.570 | 77.572 | 20.190 | 1.00 | 35.32 |
| ATOM | 1166 | CA | VAL | A | 174 | 36.677 | 77.154 | 18.813 | 1.00 | 36.57 |
| ATOM | 1167 | C | VAL | A | 174 | 37.133 | 78.311 | 17.940 | 1.00 | 37.79 |
| ATOM | 1168 | O | VAL | A | 174 | 36.676 | 79.424 | 18.108 | 1.00 | 38.26 |
| ATOM | 1169 | CB | VAL | A | 174 | 35.329 | 76.696 | 18.249 | 1.00 | 36.38 |
| ATOM | 1170 | CG1 | VAL | A | 174 | 35.462 | 76.409 | 16.776 | 1.00 | 37.71 |
| ATOM | 1171 | CG2 | VAL | A | 174 | 34.851 | 75.474 | 18.945 | 1.00 | 36.20 |
| ATOM | 1172 | N | LYS | A | 175 | 37.998 | 78.016 | 16.979 | 1.00 | 39.34 |
| ATOM | 1173 | CA | LYS | A | 175 | 38.463 | 78.984 | 15.998 | 1.00 | 40.41 |
| ATOM | 1174 | C | LYS | A | 175 | 38.191 | 78.463 | 14.599 | 1.00 | 40.91 |
| ATOM | 1175 | O | LYS | A | 175 | 38.711 | 77.420 | 14.191 | 1.00 | 40.44 |
| ATOM | 1176 | CB | LYS | A | 175 | 39.958 | 79.225 | 16.131 | 1.00 | 41.02 |
| ATOM | 1177 | CG | LYS | A | 175 | 40.310 | 80.403 | 17.018 | 1.00 | 42.53 |
| ATOM | 1178 | CD | LYS | A | 75 | 41.728 | 80.325 | 17.482 | 1.00 | 43.95 |
| ATOM | 1179 | CE | LYS | A | 175 | 42.378 | 81.679 | 17.502 | 1.00 | 45.63 |
| ATOM | 1180 | NZ | LYS | A | 175 | 42.799 | 82.132 | 16.146 | 1.00 | 44.89 |
| ATOM | 1181 | N | ILE | A | 176 | 37.372 | 79.209 | 13.870 | 1.00 | 41.50 |
| ATOM | 1182 | CA | ILE | A | 176 | 37.026 | 78.879 | 12.506 | 1.00 | 41.87 |
| ATOM | 1183 | C | ILE | A | 176 | 38.245 | 79.118 | 11.636 | 1.00 | 42.08 |
| ATOM | 1184 | O | ILE | A | 176 | 38.622 | 78.269 | 10.867 | 1.00 | 41.73 |
| ATOM | 1185 | CB | ILE | A | 176 | 35.829 | 79.699 | 12.080 | 1.00 | 42.04 |
| ATOM | 1186 | CG1 | ILE | A | 176 | 34.653 | 79.349 | 12.992 | 1.00 | 43.27 |
| ATOM | 1187 | CG2 | ILE | A | 176 | 35.447 | 79.428 | 10.616 | 1.00 | 41.99 |
| ATOM | 1188 | CD1 | ILE | A | 176 | 34.176 | 77.911 | 12.856 | 1.00 | 44.03 |
| ATOM | 1189 | N | GLU | A | 177 | 38.903 | 80.248 | 11.779 | 1.00 | 43.02 |
| ATOM | 1190 | CA | GLU | A | 177 | 40.162 | 80.444 | 11.058 | 1.00 | 44.07 |
| ATOM | 1191 | C | GLU | A | 177 | 41.207 | 80.777 | 12.077 | 1.00 | 44.83 |
| ATOM | 1192 | O | GLU | A | 177 | 40.907 | 81.329 | 13.126 | 1.00 | 44.24 |
| ATOM | 1193 | CB | GLU | A | 177 | 40.093 | 81.584 | 10.034 | 1.00 | 44.25 |
| ATOM | 1194 | CG | GLU | A | 177 | 38.809 | 81.638 | 9.220 | 1.00 | 45.05 |
| ATOM | 1195 | CD | GLU | A | 177 | 38.777 | 80.622 | 8.097 | 1.00 | 46.72 |
| ATOM | 1196 | OE1 | GLU | A | 177 | 39.841 | 80.068 | 7.766 | 1.00 | 45.39 |
| ATOM | 1197 | OE2 | GLU | A | 177 | 37.682 | 80.403 | 7.527 | 1.00 | 50.48 |
| ATOM | 1198 | N | PRO | A | 178 | 42.439 | 80.415 | 11.779 | 1.00 | 46.19 |
| ATOM | 1199 | CA | PRO | A | 178 | 43.546 | 80.666 | 12.688 | 1.00 | 46.98 |
| ATOM | 1200 | C | PRO | A | 178 | 43.634 | 82.100 | 13.117 | 1.00 | 47.64 |
| ATOM | 1201 | O | PRO | A | 178 | 43.873 | 82.382 | 14.290 | 1.00 | 46.95 |
| ATOM | 1202 | CB | PRO | A | 178 | 44.761 | 80.306 | 11.853 | 1.00 | 47.61 |
| ATOM | 1203 | CG | PRO | A | 178 | 44.262 | 79.292 | 10.878 | 1.00 | 47.37 |
| ATOM | 1204 | CD | PRO | A | 178 | 42.851 | 79.669 | 10.584 | 1.00 | 46.06 |
| ATOM | 1205 | N | ASN | A | 179 | 43.427 | 83.017 | 12.192 | 1.00 | 48.67 |
| ATOM | 1206 | CA | ASN | A | 179 | 43.621 | 84.414 | 12.547 | 1.00 | 49.57 |
| ATOM | 1207 | C | ASN | A | 179 | 42.397 | 85.113 | 13.136 | 1.00 | 49.92 |
| ATOM | 1208 | O | ASN | A | 179 | 42.503 | 86.263 | 13.570 | 1.00 | 50.50 |
| ATOM | 1209 | CB | ASN | A | 179 | 44.189 | 85.212 | 11.373 | 1.00 | 49.85 |
| ATOM | 1210 | CG | ASN | A | 179 | 43.192 | 85.421 | 10.273 | 1.00 | 49.77 |
| ATOM | 1211 | OD1 | ASN | A | 179 | 42.186 | 84.732 | 10.192 | 1.00 | 52.40 |
| ATOM | 1212 | ND2 | ASN | A | 179 | 43.486 | 86.357 | 9.396 | 1.00 | 48.53 |
| ATOM | 1213 | N | LEU | A | 180 | 41.267 | 84.418 | 13.215 | 1.00 | 49.23 |
| ATOM | 1214 | CA | LEU | A | 180 | 40.068 | 85.051 | 13.723 | 1.00 | 49.12 |
| ATOM | 1215 | C | LEU | A | 180 | 39.768 | 84.741 | 15.198 | 1.00 | 48.35 |
| ATOM | 1216 | O | LEU | A | 180 | 40.331 | 83.815 | 15.793 | 1.00 | 47.93 |
| ATOM | 1217 | CB | LEU | A | 180 | 38.862 | 84.710 | 12.835 | 1.00 | 49.65 |
| ATOM | 1218 | CG | LEU | A | 180 | 38.666 | 85.582 | 11.567 | 1.00 | 52.26 |
| ATOM | 1219 | CD1 | LEU | A | 180 | 39.327 | 86.967 | 11.695 | 1.00 | 53.45 |
| ATOM | 1220 | CD2 | LEU | A | 180 | 39.209 | 84.918 | 10.337 | 1.00 | 53.15 |
| ATOM | 1221 | N | PRO | A | 181 | 38.915 | 85.573 | 15.786 | 1.00 | 46.86 |
| ATOM | 1222 | CA | PRO | A | 181 | 38.511 | 85.431 | 17.179 | 1.00 | 46.36 |
| ATOM | 1223 | C | PRO | A | 181 | 37.861 | 84.100 | 17.474 | 1.00 | 45.21 |
| ATOM | 1224 | O | PRO | A | 181 | 37.065 | 83.622 | 16.702 | 1.00 | 46.04 |
| ATOM | 1225 | CB | PRO | A | 181 | 37.489 | 86.571 | 17.369 | 1.00 | 46.24 |
| ATOM | 1226 | CG | PRO | A | 181 | 37.866 | 87.586 | 16.359 | 1.00 | 46.31 |
| ATOM | 1227 | CD | PRO | A | 181 | 38.346 | 86.785 | 15.176 | 1.00 | 47.26 |
| ATOM | 1228 | N | SER | A | 182 | 38.194 | 83.526 | 18.612 | 1.00 | 43.72 |
| ATOM | 1229 | CA | SER | A | 182 | 37.631 | 82.264 | 19.011 | 1.00 | 43.04 |
| ATOM | 1230 | C | SER | A | 182 | 36.232 | 82.468 | 19.605 | 1.00 | 42.27 |
| ATOM | 1231 | O | SER | A | 182 | 35.922 | 83.501 | 20.169 | 1.00 | 41.67 |
| ATOM | 1232 | CB | SER | A | 182 | 38.561 | 81.612 | 20.025 | 1.00 | 42.97 |
| ATOM | 1233 | OG | SER | A | 182 | 38.449 | 82.245 | 21.280 | 1.00 | 42.30 |
| ATOM | 1234 | N | TYR | A | 183 | 35.369 | 81.490 | 19.424 | 1.00 | 41.67 |
| ATOM | 1235 | CA | TYR | A | 183 | 34.052 | 81.535 | 20.009 | 1.00 | 41.15 |
| ATOM | 1236 | C | TYR | A | 183 | 34.135 | 80.676 | 21.271 | 1.00 | 39.87 |
| ATOM | 1237 | O | TYR | A | 183 | 34.633 | 79.553 | 21.207 | 1.00 | 39.01 |
| ATOM | 1238 | CB | TYR | A | 183 | 33.021 | 80.925 | 19.061 | 1.00 | 41.56 |
| ATOM | 1239 | CG | TYR | A | 183 | 32.862 | 81.629 | 17.726 | 1.00 | 44.63 |
| ATOM | 1240 | CD1 | TYR | A | 183 | 33.729 | 81.369 | 16.672 | 1.00 | 46.43 |
| ATOM | 1241 | CD2 | TYR | A | 183 | 31.825 | 82.533 | 17.509 | 1.00 | 46.16 |
| ATOM | 1242 | CE1 | TYR | A | 183 | 33.571 | 81.989 | 15.454 | 1.00 | 48.20 |
| ATOM | 1243 | CE2 | TYR | A | 183 | 31.674 | 83.177 | 16.290 | 1.00 | 46.24 |
| ATOM | 1244 | CZ | TYR | A | 183 | 32.544 | 82.906 | 15.271 | 1.00 | 48.83 |
| ATOM | 1245 | OH | TYR | A | 183 | 32.391 | 83.530 | 14.042 | 1.00 | 51.62 |
| ATOM | 1246 | N | ARG | A | 184 | 33.620 | 81.195 | 22.391 | 1.00 | 38.83 |
| ATOM | 1247 | CA | ARG | A | 184 | 33.636 | 80.505 | 23.676 | 1.00 | 37.75 |
| ATOM | 1248 | C | ARG | A | 184 | 32.479 | 79.526 | 23.737 | 1.00 | 36.66 |
| ATOM | 1249 | O | ARG | A | 184 | 31.350 | 79.911 | 23.575 | 1.00 | 36.62 |
| ATOM | 1250 | CB | ARG | A | 184 | 33.544 | 81.535 | 24.816 | 1.00 | 38.30 |
| ATOM | 1251 | CG | ARG | A | 184 | 33.626 | 80.933 | 26.220 | 1.00 | 39.20 |
| ATOM | 1252 | CD | ARG | A | 184 | 34.091 | 81.882 | 27.312 | 1.00 | 38.92 |
| ATOM | 1253 | NE | ARG | A | 184 | 34.047 | 81.241 | 28.625 | 1.00 | 39.00 |
| ATOM | 1254 | CZ | ARG | A | 184 | 34.541 | 81.773 | 29.759 | 1.00 | 40.63 |
| ATOM | 1255 | NH1 | ARG | A | 184 | 35.125 | 82.966 | 29.768 | 1.00 | 40.89 |
| ATOM | 1256 | NH2 | ARG | A | 184 | 34.460 | 81.096 | 30.891 | 1.00 | 38.62 |
| ATOM | 1257 | N | ILE | A | 185 | 32.745 | 78.250 | 23.968 | 1.00 | 35.61 |
| ATOM | 1258 | CA | ILE | A | 185 | 31.674 | 77.269 | 23.984 | 1.00 | 35.15 |
| ATOM | 1259 | C | ILE | A | 185 | 31.088 | 77.004 | 25.377 | 1.00 | 35.45 |
| ATOM | 1260 | O | ILE | A | 185 | 29.907 | 76.689 | 25.530 | 1.00 | 35.36 |
| ATOM | 1261 | CB | ILE | A | 185 | 32.182 | 75.952 | 23.416 | 1.00 | 34.42 |
| ATOM | 1262 | CG1 | ILE | A | 185 | 32.793 | 76.150 | 22.038 | 1.00 | 35.36 |
| ATOM | 1263 | CG2 | ILE | A | 185 | 31.064 | 74.939 | 23.372 | 1.00 | 33.33 |
| ATOM | 1264 | CD1 | ILE | A | 185 | 31.872 | 76.837 | 20.993 | 1.00 | 33.99 |
| ATOM | 1265 | N | THR | A | 186 | 31.923 | 77.091 | 26.393 | 1.00 | 36.11 |
| ATOM | 1266 | CA | THR | A | 186 | 31.481 | 76.745 | 27.742 | 1.00 | 36.38 |
| ATOM | 1267 | C | THR | A | 186 | 31.870 | 77.830 | 28.715 | 1.00 | 36.91 |
| ATOM | 1268 | O | THR | A | 186 | 32.751 | 78.631 | 28.448 | 1.00 | 36.47 |
| ATOM | 1269 | CB | THR | A | 186 | 32.021 | 75.344 | 28.211 | 1.00 | 36.38 |
| ATOM | 1270 | OG1 | THR | A | 186 | 33.453 | 75.330 | 28.284 | 1.00 | 33.71 |
| ATOM | 1271 | CG2 | THR | A | 186 | 31.666 | 74.261 | 27.218 | 1.00 | 37.13 |
| ATOM | 1272 | N | TRP | A | 187 | 31.192 | 77.852 | 29.843 | 1.00 | 37.99 |
| ATOM | 1273 | CA | TRP | A | 187 | 31.405 | 78.906 | 30.820 | 1.00 | 39.81 |
| ATOM | 1274 | C | TRP | A | 187 | 31.515 | 78.390 | 32.228 | 1.00 | 39.36 |
| ATOM | 1275 | O | TRP | A | 187 | 31.762 | 79.160 | 33.139 | 1.00 | 40.25 |
| ATOM | 1276 | CB | TRP | A | 187 | 30.245 | 79.910 | 30.766 | 1.00 | 40.15 |
| ATOM | 1277 | CG | TRP | A | 187 | 30.143 | 80.591 | 29.426 | 1.00 | 43.68 |
| ATOM | 1278 | CD1 | TRP | A | 187 | 29.603 | 80.068 | 28.266 | 1.00 | 44.15 |
| ATOM | 1279 | CD2 | TRP | A | 187 | 30.637 | 81.892 | 29.082 | 1.00 | 45.22 |
| ATOM | 1280 | NE1 | TRP | A | 187 | 29.711 | 80.986 | 27.249 | 1.00 | 44.94 |
| ATOM | 1281 | CE2 | TRP | A | 187 | 30.339 | 82.110 | 27.720 | 1.00 | 43.72 |
| ATOM | 1282 | CE3 | TRP | A | 187 | 31.283 | 82.908 | 29.795 | 1.00 | 47.78 |
| ATOM | 1283 | CZ2 | TRP | A | 187 | 30.657 | 83.294 | 27.062 | 1.00 | 45.92 |
| ATOM | 1284 | CZ3 | TRP | A | 187 | 31.607 | 84.092 | 29.132 | 1.00 | 48.57 |
| ATOM | 1285 | CH2 | TRP | A | 187 | 31.287 | 84.270 | 27.772 | 1.00 | 47.33 |
| ATOM | 1286 | N | THR | A | 188 | 31.373 | 77.090 | 32.390 | 1.00 | 38.68 |
| ATOM | 1287 | CA | THR | A | 188 | 31.350 | 76.462 | 33.684 | 1.00 | 38.37 |
| ATOM | 1288 | C | THR | A | 188 | 32.706 | 75.969 | 34.141 | 1.00 | 38.26 |
| ATOM | 1289 | O | THR | A | 188 | 32.833 | 75.440 | 35.246 | 1.00 | 38.27 |
| ATOM | 1290 | CB | THR | A | 188 | 30.458 | 75.251 | 33.568 | 1.00 | 38.31 |
| ATOM | 1291 | OG1 | THR | A | 188 | 30.904 | 74.443 | 32.463 | 1.00 | 37.24 |
| ATOM | 1292 | CG2 | THR | A | 188 | 29.049 | 75.667 | 33.224 | 1.00 | 37.94 |
| ATOM | 1293 | N | GLY | A | 189 | 33.710 | 76.093 | 33.283 | 1.00 | 37.93 |
| ATOM | 1294 | CA | GLY | A | 189 | 35.023 | 75.565 | 33.606 | 1.00 | 38.12 |
| ATOM | 1295 | C | GLY | A | 189 | 35.476 | 76.074 | 34.957 | 1.00 | 38.04 |
| ATOM | 1296 | O | GLY | A | 189 | 35.295 | 77.246 | 35.247 | 1.00 | 38.63 |
| ATOM | 1297 | N | LYS | A | 190 | 36.074 | 75.209 | 35.769 | 1.00 | 37.97 |
| ATOM | 1298 | CA | LYS | A | 190 | 36.541 | 75.583 | 37.117 | 1.00 | 38.44 |
| ATOM | 1299 | C | LYS | A | 190 | 37.629 | 74.604 | 37.567 | 1.00 | 37.55 |
| ATOM | 1300 | O | LYS | A | 190 | 37.393 | 73.398 | 37.717 | 1.00 | 36.54 |
| ATOM | 1301 | CB | LYS | A | 190 | 35.346 | 75.597 | 38.124 | 1.00 | 38.40 |
| ATOM | 1302 | CG | LYS | A | 190 | 35.670 | 76.047 | 39.594 | 1.00 | 41.68 |
| ATOM | 1303 | CD | LYS | A | 190 | 34.366 | 76.151 | 40.490 | 1.00 | 44.07 |
| ATOM | 1304 | CE | LYS | A | 190 | 34.678 | 76.437 | 41.984 | 1.00 | 45.90 |
| ATOM | 1305 | NZ | LYS | A | 190 | 33.447 | 76.400 | 42.888 | 1.00 | 44.46 |
| ATOM | 1306 | N | GLU | A | 191 | 38.822 | 75.135 | 37.792 | 1.00 | 37.97 |
| ATOM | 1307 | CA | GLU | A | 191 | 39.978 | 74.324 | 38.182 | 1.00 | 37.95 |
| ATOM | 1308 | C | GLU | A | 191 | 39.641 | 73.268 | 39.232 | 1.00 | 36.73 |
| ATOM | 1309 | O | GLU | A | 191 | 38.993 | 73.560 | 40.232 | 1.00 | 35.27 |
| ATOM | 1310 | CB | GLU | A | 191 | 41.127 | 75.210 | 38.673 | 1.00 | 39.08 |
| ATOM | 1311 | CG | GLU | A | 191 | 42.497 | 74.512 | 38.619 | 1.00 | 42.11 |
| ATOM | 1312 | CD | GLU | A | 191 | 43.628 | 75.383 | 39.148 | 1.00 | 45.78 |
| ATOM | 1313 | OE1 | GLU | A | 191 | 43.375 | 76.562 | 39.474 | 1.00 | 49.77 |
| ATOM | 1314 | OE2 | GLU | A | 191 | 44.760 | 74.886 | 39.259 | 1.00 | 47.21 |
| ATOM | 1315 | N | ASP | A | 192 | 40.082 | 72.036 | 38.977 | 1.00 | 35.55 |
| ATOM | 1316 | CA | ASP | A | 192 | 39.835 | 70.903 | 39.875 | 1.00 | 35.43 |
| ATOM | 1317 | C | ASP | A | 192 | 38.394 | 70.518 | 40.097 | 1.00 | 34.50 |
| ATOM | 1318 | O | ASP | A | 192 | 38.127 | 69.640 | 40.892 | 1.00 | 33.73 |
| ATOM | 1319 | CB | ASP | A | 192 | 40.419 | 71.185 | 41.264 | 1.00 | 36.28 |
| ATOM | 1320 | CG | ASP | A | 192 | 41.923 | 71.224 | 41.257 | 1.00 | 37.00 |
| ATOM | 1321 | OD1 | ASP | A | 192 | 42.539 | 70.535 | 40.429 | 1.00 | 38.57 |
| ATOM | 1322 | OD2 | ASP | A | 192 | 42.571 | 71.911 | 42.061 | 1.00 | 41.87 |
| ATOM | 1323 | N | ILE | A | 193 | 37.448 | 71.130 | 39.395 | 1.00 | 33.93 |
| ATOM | 1324 | CA | ILE | A | 193 | 36.062 | 70.880 | 39.718 | 1.00 | 32.73 |
| ATOM | 1325 | C | ILE | A | 193 | 35.184 | 70.612 | 38.491 | 1.00 | 31.43 |
| ATOM | 1326 | O | ILE | A | 193 | 34.494 | 69.605 | 38.441 | 1.00 | 30.34 |
| ATOM | 1327 | CB | ILE | A | 193 | 35.573 | 72.043 | 40.593 | 1.00 | 33.45 |
| ATOM | 1328 | CG1 | ILE | A | 193 | 36.150 | 71.878 | 42.019 | 1.00 | 36.91 |
| ATOM | 1329 | CG2 | ILE | A | 193 | 34.056 | 72.077 | 40.686 | 1.00 | 34.61 |
| ATOM | 1330 | CD1 | ILE | A | 193 | 36.455 | 73.166 | 42.759 | 1.00 | 40.22 |
| ATOM | 1331 | N | ILE | A | 194 | 35.200 | 71.523 | 37.527 | 1.00 | 30.62 |
| ATOM | 1332 | CA | ILE | A | 194 | 34.448 | 71.376 | 36.312 | 1.00 | 30.28 |
| ATOM | 1333 | C | ILE | A | 194 | 35.435 | 71.323 | 35.166 | 1.00 | 29.31 |
| ATOM | 1334 | O | ILE | A | 194 | 36.236 | 72.244 | 34.974 | 1.00 | 28.40 |
| ATOM | 1335 | CB | ILE | A | 194 | 33.446 | 72.525 | 36.102 | 1.00 | 30.93 |
| ATOM | 1336 | CG1 | ILE | A | 194 | 32.462 | 72.643 | 37.267 | 1.00 | 31.11 |
| ATOM | 1337 | CG2 | ILE | A | 194 | 32.662 | 72.281 | 34.828 | 1.00 | 32.14 |
| ATOM | 1338 | CD1 | ILE | A | 194 | 31.795 | 71.369 | 37.640 | 1.00 | 32.16 |
| ATOM | 1339 | N | TYR | A | 195 | 35.408 | 70.210 | 34.443 | 1.00 | 28.47 |
| ATOM | 1340 | CA | TYR | A | 195 | 36.295 | 70.025 | 33.314 | 1.00 | 28.34 |
| ATOM | 1341 | C | TYR | A | 195 | 35.475 | 69.894 | 32.017 | 1.00 | 27.52 |
| ATOM | 1342 | O | TYR | A | 195 | 34.711 | 68.952 | 31.840 | 1.00 | 27.00 |
| ATOM | 1343 | CB | TYR | A | 195 | 37.147 | 68.746 | 33.481 | 1.00 | 28.77 |
| ATOM | 1344 | CG | TYR | A | 195 | 37.973 | 68.569 | 34.730 | 1.00 | 28.86 |
| ATOM | 1345 | CD1 | TYR | A | 195 | 37.375 | 68.485 | 35.982 | 1.00 | 31.13 |
| ATOM | 1346 | CD2 | TYR | A | 195 | 39.368 | 68.408 | 34.650 | 1.00 | 29.50 |
| ATOM | 1347 | CE1 | TYR | A | 195 | 38.138 | 68.291 | 37.125 | 1.00 | 30.70 |
| ATOM | 1348 | CE2 | TYR | A | 195 | 40.136 | 68.210 | 35.773 | 1.00 | 28.60 |
| ATOM | 1349 | CZ | TYR | A | 195 | 39.515 | 68.141 | 37.014 | 1.00 | 31.43 |
| ATOM | 1350 | OH | TYR | A | 195 | 40.250 | 67.942 | 38.161 | 1.00 | 30.49 |
| ATOM | 1351 | N | ASN | A | 196 | 35.638 | 70.841 | 31.124 | 1.00 | 26.73 |
| ATOM | 1352 | CA | ASN | A | 196 | 34.971 | 70.787 | 29.832 | 1.00 | 27.22 |
| ATOM | 1353 | C | ASN | A | 196 | 35.995 | 70.465 | 28.744 | 1.00 | 26.56 |
| ATOM | 1354 | O | ASN | A | 196 | 36.911 | 71.241 | 28.528 | 1.00 | 26.95 |
| ATOM | 1355 | CB | ASN | A | 196 | 34.270 | 72.110 | 29.517 | 1.00 | 26.46 |
| ATOM | 1356 | CG | ASN | A | 196 | 33.210 | 72.479 | 30.560 | 1.00 | 27.41 |
| ATOM | 1357 | OD1 | ASN | A | 196 | 32.132 | 71.847 | 30.661 | 1.00 | 26.50 |
| ATOM | 1358 | ND2 | ASN | A | 196 | 33.503 | 73.528 | 31.334 | 1.00 | 26.57 |
| ATOM | 1359 | N | GLY | A | 197 | 35.866 | 69.292 | 28.134 | 1.00 | 25.76 |
| ATOM | 1360 | CA | GLY | A | 197 | 36.693 | 68.911 | 27.014 | 1.00 | 26.04 |
| ATOM | 1361 | C | GLY | A | 197 | 38.060 | 68.332 | 27.353 | 1.00 | 25.60 |
| ATOM | 1362 | O | GLY | A | 197 | 38.854 | 68.199 | 26.466 | 1.00 | 26.73 |
| ATOM | 1363 | N | ILE | A | 198 | 38.303 | 68.025 | 28.617 | 1.00 | 25.23 |
| ATOM | 1364 | CA | ILE | A | 198 | 39.517 | 67.405 | 29.102 | 1.00 | 25.68 |
| ATOM | 1365 | C | ILE | A | 198 | 39.075 | 66.541 | 30.259 | 1.00 | 25.94 |
| ATOM | 1366 | O | ILE | A | 198 | 38.012 | 66.777 | 30.826 | 1.00 | 26.05 |
| ATOM | 1367 | CB | ILE | A | 198 | 40.579 | 68.431 | 29.589 | 1.00 | 25.47 |
| ATOM | 1368 | CG1 | ILE | A | 198 | 39.939 | 69.442 | 30.542 | 1.00 | 26.41 |
| ATOM | 1369 | CG2 | ILE | A | 198 | 41.225 | 69.120 | 28.403 | 1.00 | 26.50 |
| ATOM | 1370 | CD1 | ILE | A | 198 | 40.929 | 70.437 | 31.147 | 1.00 | 27.59 |
| ATOM | 1371 | N | THR | A | 199 | 39.877 | 65.535 | 30.588 | 1.00 | 25.78 |
| ATOM | 1372 | CA | THR | A | 199 | 39.521 | 64.555 | 31.581 | 1.00 | 27.13 |
| ATOM | 1373 | C | THR | A | 199 | 40.044 | 64.987 | 32.949 | 1.00 | 27.58 |
| ATOM | 1374 | O | THR | A | 199 | 40.994 | 65.765 | 33.006 | 1.00 | 27.38 |
| ATOM | 1375 | CB | THR | A | 199 | 40.183 | 63.217 | 31.240 | 1.00 | 27.56 |
| ATOM | 1376 | OG1 | THR | A | 199 | 41.546 | 63.434 | 30.778 | 1.00 | 28.44 |
| ATOM | 1377 | CG2 | THR | A | 199 | 39.459 | 62.527 | 30.071 | 1.00 | 29.85 |
| ATOM | 1378 | N | ASP | A | 200 | 39.407 | 64.484 | 34.018 | 1.00 | 27.06 |
| ATOM | 1379 | CA | ASP | A | 200 | 39.938 | 64.629 | 35.380 | 1.00 | 27.25 |
| ATOM | 1380 | C | ASP | A | 200 | 41.008 | 63.560 | 35.484 | 1.00 | 26.77 |
| ATOM | 1381 | O | ASP | A | 200 | 41.346 | 62.942 | 34.447 | 1.00 | 26.51 |
| ATOM | 1382 | CB | ASP | A | 200 | 38.850 | 64.530 | 36.456 | 1.00 | 27.28 |
| ATOM | 1383 | CG | ASP | A | 200 | 38.352 | 63.124 | 36.651 | 1.00 | 28.95 |
| ATOM | 1384 | OD1 | ASP | A | 200 | 38.616 | 62.275 | 35.776 | 1.00 | 25.86 |
| ATOM | 1385 | OD2 | ASP | A | 200 | 37.708 | 62.761 | 37.671 | 1.00 | 30.54 |
| ATOM | 1386 | N | TRP | A | 201 | 41.602 | 63.378 | 36.672 | 1.00 | 25.97 |
| ATOM | 1387 | CA | TRP | A | 201 | 42.704 | 62.420 | 36.831 | 1.00 | 24.90 |
| ATOM | 1388 | C | TRP | A | 201 | 42.321 | 60.967 | 36.477 | 1.00 | 23.95 |
| ATOM | 1389 | O | TRP | A | 201 | 43.038 | 60.323 | 35.690 | 1.00 | 22.08 |
| ATOM | 1390 | CB | TRP | A | 201 | 43.338 | 62.481 | 38.241 | 1.00 | 25.55 |
| ATOM | 1391 | CG | TRP | A | 201 | 44.643 | 61.680 | 38.351 | 1.00 | 23.04 |
| ATOM | 1392 | CD1 | TRP | A | 201 | 45.897 | 62.179 | 38.282 | 1.00 | 22.01 |
| ATOM | 1393 | CD2 | TRP | A | 201 | 44.789 | 60.258 | 38.521 | 1.00 | 19.68 |
| ATOM | 1394 | NE1 | TRP | A | 201 | 46.815 | 61.169 | 38.406 | 1.00 | 22.38 |
| ATOM | 1395 | CE2 | TRP | A | 201 | 46.156 | 59.978 | 38.550 | 1.00 | 21.58 |
| ATOM | 1396 | CE3 | TRP | A | 201 | 43.903 | 59.201 | 38.644 | 1.00 | 18.42 |
| ATOM | 1397 | CZ2 | TRP | A | 201 | 46.652 | 58.694 | 38.682 | 1.00 | 21.85 |
| ATOM | 1398 | CZ3 | TRP | A | 201 | 44.394 | 57.931 | 38.773 | 1.00 | 20.23 |
| ATOM | 1399 | CH2 | TRP | A | 201 | 45.764 | 57.684 | 38.804 | 1.00 | 21.03 |
| ATOM | 1400 | N | VAL | A | 202 | 41.199 | 60.470 | 37.007 | 1.00 | 22.84 |
| ATOM | 1401 | CA | VAL | A | 202 | 40.846 | 59.096 | 36.752 | 1.00 | 23.88 |
| ATOM | 1402 | C | VAL | A | 202 | 40.493 | 58.884 | 35.292 | 1.00 | 22.91 |
| ATOM | 1403 | O | VAL | A | 202 | 40.935 | 57.922 | 34.740 | 1.00 | 21.58 |
| ATOM | 1404 | CB | VAL | A | 202 | 39.592 | 58.483 | 37.380 | 1.00 | 24.76 |
| ATOM | 1405 | CG1 | VAL | A | 202 | 39.939 | 57.345 | 38.239 | 1.00 | 24.92 |
| ATOM | 1406 | CG2 | VAL | A | 202 | 38.597 | 59.452 | 37.914 | 1.00 | 27.11 |
| ATOM | 1407 | N | TYR | A | 203 | 39.660 | 59.750 | 34.724 | 1.00 | 23.45 |
| ATOM | 1408 | CA | TYR | A | 203 | 39.307 | 59.635 | 33.314 | 1.00 | 23.95 |
| ATOM | 1409 | C | TYR | A | 203 | 40.576 | 59.737 | 32.420 | 1.00 | 24.22 |
| ATOM | 1410 | O | TYR | A | 203 | 40.685 | 59.075 | 31.390 | 1.00 | 24.25 |
| ATOM | 1411 | CB | TYR | A | 203 | 38.224 | 60.637 | 32.910 | 1.00 | 23.86 |
| ATOM | 1412 | CG | TYR | A | 203 | 36.791 | 60.101 | 33.028 | 1.00 | 23.56 |
| ATOM | 1413 | CD1 | TYR | A | 203 | 36.053 | 60.218 | 34.209 | 1.00 | 23.85 |
| ATOM | 1414 | CD2 | TYR | A | 203 | 36.170 | 59.507 | 31.949 | 1.00 | 24.49 |
| ATOM | 1415 | CE1 | TYR | A | 203 | 34.766 | 59.732 | 34.277 | 1.00 | 23.23 |
| ATOM | 1416 | CE2 | TYR | A | 203 | 34.920 | 58.983 | 32.035 | 1.00 | 23.15 |
| ATOM | 1417 | CZ | TYR | A | 203 | 34.204 | 59.133 | 33.173 | 1.00 | 23.57 |
| ATOM | 1418 | OH | TYR | A | 203 | 32.935 | 58.637 | 33.204 | 1.00 | 25.76 |
| ATOM | 1419 | N | GLU | A | 204 | 41.539 | 60.563 | 32.811 | 1.00 | 23.96 |
| ATOM | 1420 | CA | GLU | A | 204 | 42.739 | 60.661 | 31.999 | 1.00 | 24.49 |
| ATOM | 1421 | C | GLU | A | 204 | 43.529 | 59.364 | 32.029 | 1.00 | 24.38 |
| ATOM | 1422 | O | GLU | A | 204 | 43.912 | 58.812 | 30.990 | 1.00 | 25.04 |
| ATOM | 1423 | CB | GLU | A | 204 | 43.678 | 61.760 | 32.468 | 1.00 | 24.31 |
| ATOM | 1424 | CG | GLU | A | 204 | 45.068 | 61.577 | 31.867 | 1.00 | 25.99 |
| ATOM | 1425 | CD | GLU | A | 204 | 46.009 | 62.723 | 32.142 | 1.00 | 23.67 |
| ATOM | 1426 | OE1 | GLU | A | 204 | 45.541 | 63.726 | 32.686 | 1.00 | 28.19 |
| ATOM | 1427 | OE2 | GLU | A | 204 | 47.216 | 62.611 | 31.846 | 1.00 | 22.68 |
| ATOM | 1428 | N | GLU | A | 205 | 43.745 | 58.868 | 33.230 | 1.00 | 23.35 |
| ATOM | 1429 | CA | GLU | A | 205 | 44.647 | 57.750 | 33.433 | 1.00 | 23.85 |
| ATOM | 1430 | C | GLU | A | 205 | 44.075 | 56.390 | 33.112 | 1.00 | 24.27 |
| ATOM | 1431 | O | GLU | A | 205 | 44.758 | 55.558 | 32.545 | 1.00 | 24.01 |
| ATOM | 1432 | CB | GLU | A | 205 | 45.109 | 57.783 | 34.873 | 1.00 | 22.48 |
| ATOM | 1433 | CG | GLU | A | 205 | 46.128 | 56.779 | 35.260 | 1.00 | 24.17 |
| ATOM | 1434 | CD | GLU | A | 205 | 47.329 | 56.653 | 34.337 | 1.00 | 24.44 |
| ATOM | 1435 | OE1 | GLU | A | 205 | 47.716 | 57.564 | 33.565 | 1.00 | 24.45 |
| ATOM | 1436 | OE2 | GLU | A | 205 | 47.903 | 55.581 | 34.421 | 1.00 | 24.40 |
| ATOM | 1437 | N | GLU | A | 206 | 42.826 | 56.182 | 33.504 | 1.00 | 25.05 |
| ATOM | 1438 | CA | GLU | A | 206 | 42.214 | 54.889 | 33.502 | 1.00 | 25.30 |
| ATOM | 1439 | C | GLU | A | 206 | 41.080 | 54.703 | 32.512 | 1.00 | 26.38 |
| ATOM | 1440 | O | GLU | A | 206 | 40.927 | 53.599 | 32.018 | 1.00 | 26.34 |
| ATOM | 1441 | CB | GLU | A | 206 | 41.673 | 54.606 | 34.908 | 1.00 | 25.85 |
| ATOM | 1442 | CG | GLU | A | 206 | 42.711 | 54.739 | 36.005 | 1.00 | 25.12 |
| ATOM | 1443 | CD | GLU | A | 206 | 43.655 | 53.554 | 36.079 | 1.00 | 26.60 |
| ATOM | 1444 | OE1 | GLU | A | 206 | 43.635 | 52.718 | 35.154 | 1.00 | 24.07 |
| ATOM | 1445 | OE2 | GLU | A | 206 | 44.383 | 53.434 | 37.097 | 1.00 | 23.28 |
| ATOM | 1446 | N | VAL | A | 207 | 40.295 | 55.742 | 32.212 | 1.00 | 26.89 |
| ATOM | 1447 | CA | VAL | A | 207 | 39.156 | 55.525 | 31.332 | 1.00 | 26.90 |
| ATOM | 1448 | C | VAL | A | 207 | 39.452 | 55.829 | 29.884 | 1.00 | 27.08 |
| ATOM | 1449 | O | VAL | A | 207 | 39.335 | 54.939 | 29.084 | 1.00 | 27.79 |
| ATOM | 1450 | CB | VAL | A | 207 | 37.865 | 56.185 | 31.789 | 1.00 | 27.63 |
| ATOM | 1451 | CG1 | VAL | A | 207 | 36.726 | 55.634 | 30.999 | 1.00 | 24.75 |
| ATOM | 1452 | CG2 | VAL | A | 207 | 37.584 | 55.880 | 33.255 | 1.00 | 26.16 |
| ATOM | 1453 | N | PHE | A | 208 | 39.868 | 57.041 | 29.552 | 1.00 | 26.16 |
| ATOM | 1454 | CA | PHE | A | 208 | 40.180 | 57.379 | 28.168 | 1.00 | 25.94 |
| ATOM | 1455 | C | PHE | A | 208 | 41.655 | 57.189 | 27.784 | 1.00 | 25.74 |
| ATOM | 1456 | O | PHE | A | 208 | 41.964 | 57.138 | 26.599 | 1.00 | 25.57 |
| ATOM | 1457 | CB | PHE | A | 208 | 39.887 | 58.846 | 27.841 | 1.00 | 25.16 |
| ATOM | 1458 | CG | PHE | A | 208 | 38.453 | 59.246 | 27.884 | 1.00 | 25.82 |
| ATOM | 1459 | CD1 | PHE | A | 208 | 37.441 | 58.346 | 28.011 | 1.00 | 28.40 |
| ATOM | 1460 | CD2 | PHE | A | 208 | 38.126 | 60.587 | 27.774 | 1.00 | 28.74 |
| ATOM | 1461 | CE1 | PHE | A | 208 | 36.138 | 58.765 | 28.026 | 1.00 | 27.18 |
| ATOM | 1462 | CE2 | PHE | A | 208 | 36.841 | 61.009 | 27.809 | 1.00 | 27.47 |
| ATOM | 1463 | CZ | PHE | A | 208 | 35.842 | 60.088 | 27.918 | 1.00 | 28.83 |
| ATOM | 1464 | N | SER | A | 209 | 42.562 | 57.097 | 28.755 | 1.00 | 25.92 |
| ATOM | 1465 | CA | SER | A | 209 | 44.025 | 57.083 | 28.461 | 1.00 | 26.32 |
| ATOM | 1466 | C | SER | A | 209 | 44.408 | 58.267 | 27.598 | 1.00 | 25.78 |
| ATOM | 1467 | O | SER | A | 209 | 45.199 | 58.162 | 26.634 | 1.00 | 25.56 |
| ATOM | 1468 | CB | SER | A | 209 | 44.491 | 55.779 | 27.788 | 1.00 | 26.23 |
| ATOM | 1469 | OG | SER | A | 209 | 44.260 | 54.669 | 28.654 | 1.00 | 28.15 |
| ATOM | 1470 | N | ALA | A | 210 | 43.873 | 59.415 | 27.964 | 1.00 | 25.14 |
| ATOM | 1471 | CA | ALA | A | 210 | 44.112 | 60.612 | 27.206 | 1.00 | 25.32 |
| ATOM | 1472 | C | ALA | A | 210 | 43.541 | 61.773 | 27.956 | 1.00 | 24.94 |
| ATOM | 1473 | O | ALA | A | 210 | 42.607 | 61.602 | 28.749 | 1.00 | 25.25 |
| ATOM | 1474 | CB | ALA | A | 210 | 43.427 | 60.472 | 25.805 | 1.00 | 26.30 |
| ATOM | 1475 | N | TYR | A | 211 | 44.117 | 62.945 | 27.746 | 1.00 | 25.21 |
| ATOM | 1476 | CA | TYR | A | 211 | 43.635 | 64.183 | 28.341 | 1.00 | 25.71 |
| ATOM | 1477 | C | TYR | A | 211 | 42.431 | 64.718 | 27.600 | 1.00 | 26.13 |
| ATOM | 1478 | O | TYR | A | 211 | 41.541 | 65.335 | 28.189 | 1.00 | 27.81 |
| ATOM | 1479 | CB | TYR | A | 211 | 44.709 | 65.241 | 28.293 | 1.00 | 25.39 |
| ATOM | 1480 | CG | TYR | A | 211 | 44.486 | 66.458 | 29.201 | 1.00 | 26.84 |
| ATOM | 1481 | CD1 | TYR | A | 211 | 43.726 | 66.388 | 30.368 | 1.00 | 28.16 |
| ATOM | 1482 | CD2 | TYR | A | 211 | 45.103 | 67.649 | 28.912 | 1.00 | 28.94 |
| ATOM | 1483 | CE1 | TYR | A | 211 | 43.580 | 67.503 | 31.206 | 1.00 | 27.97 |
| ATOM | 1484 | CE2 | TYR | A | 211 | 44.986 | 68.738 | 29.736 | 1.00 | 31.54 |
| ATOM | 1485 | CZ | TYR | A | 211 | 44.217 | 68.653 | 30.892 | 1.00 | 29.76 |
| ATOM | 1486 | OH | TYR | A | 211 | 44.094 | 69.774 | 31.654 | 1.00 | 28.46 |
| ATOM | 1487 | N | SER | A | 212 | 42.393 | 64.494 | 26.297 | 1.00 | 26.81 |
| ATOM | 1488 | CA | SER | A | 212 | 41.339 | 65.067 | 25.490 | 1.00 | 26.75 |
| ATOM | 1489 | C | SER | A | 212 | 39.978 | 64.470 | 25.719 | 1.00 | 26.28 |
| ATOM | 1490 | O | SER | A | 212 | 39.837 | 63.264 | 25.890 | 1.00 | 24.94 |
| ATOM | 1491 | CB | SER | A | 212 | 41.627 | 64.896 | 24.022 | 1.00 | 27.36 |
| ATOM | 1492 | OG | SER | A | 212 | 40.665 | 65.666 | 23.334 | 1.00 | 30.68 |
| ATOM | 1493 | N | ALA | A | 213 | 38.966 | 65.333 | 25.717 | 1.00 | 26.33 |
| ATOM | 1494 | CA | ALA | A | 213 | 37.614 | 64.864 | 25.821 | 1.00 | 26.95 |
| ATOM | 1495 | C | ALA | A | 213 | 36.758 | 65.656 | 24.847 | 1.00 | 27.47 |
| ATOM | 1496 | O | ALA | A | 213 | 35.665 | 66.116 | 25.190 | 1.00 | 26.21 |
| ATOM | 1497 | CB | ALA | A | 213 | 37.119 | 65.001 | 27.240 | 1.00 | 28.10 |
| ATOM | 1498 | N | LEU | A | 214 | 37.297 | 65.781 | 23.628 | 1.00 | 27.83 |
| ATOM | 1499 | CA | LEU | A | 214 | 36.675 | 66.459 | 22.488 | 1.00 | 28.52 |
| ATOM | 1500 | C | LEU | A | 214 | 36.680 | 65.512 | 21.249 | 1.00 | 28.36 |
| ATOM | 1501 | O | LEU | A | 214 | 37.685 | 64.886 | 20.948 | 1.00 | 26.65 |
| ATOM | 1502 | CB | LEU | A | 214 | 37.477 | 67.719 | 22.104 | 1.00 | 29.14 |
| ATOM | 1503 | CG | LEU | A | 214 | 37.670 | 68.866 | 23.103 | 1.00 | 30.74 |
| ATOM | 1504 | CD1 | LEU | A | 214 | 38.515 | 69.912 | 22.501 | 1.00 | 32.71 |
| ATOM | 1505 | CD2 | LEU | A | 214 | 36.363 | 69.457 | 23.442 | 1.00 | 31.48 |
| ATOM | 1506 | N | TRP | A | 215 | 35.581 | 65.472 | 20.504 | 1.00 | 28.74 |
| ATOM | 1507 | CA | TRP | A | 215 | 35.499 | 64.653 | 19.296 | 1.00 | 29.38 |
| ATOM | 1508 | C | TRP | A | 215 | 34.829 | 65.419 | 18.180 | 1.00 | 29.51 |
| ATOM | 1509 | O | TRP | A | 215 | 33.624 | 65.644 | 18.208 | 1.00 | 28.64 |
| ATOM | 1510 | CB | TRP | A | 215 | 34.719 | 63.378 | 19.571 | 1.00 | 28.75 |
| ATOM | 1511 | CG | TRP | A | 215 | 35.313 | 62.597 | 20.678 | 1.00 | 28.51 |
| ATOM | 1512 | CD1 | TRP | A | 215 | 36.238 | 61.608 | 20.582 | 1.00 | 29.30 |
| ATOM | 1513 | CD2 | TRP | A | 215 | 35.053 | 62.765 | 22.084 | 1.00 | 30.05 |
| ATOM | 1514 | NE1 | TRP | A | 215 | 36.555 | 61.125 | 21.841 | 1.00 | 27.30 |
| ATOM | 1515 | CE2 | TRP | A | 215 | 35.844 | 61.818 | 22.778 | 1.00 | 28.78 |
| ATOM | 1516 | CE3 | TRP | A | 215 | 34.225 | 63.612 | 22.824 | 1.00 | 29.11 |
| ATOM | 1517 | CZ2 | TRP | A | 215 | 35.825 | 61.692 | 24.165 | 1.00 | 27.86 |
| ATOM | 1518 | CZ3 | TRP | A | 215 | 34.204 | 63.486 | 24.192 | 1.00 | 29.94 |
| ATOM | 1519 | CH2 | TRP | A | 215 | 35.003 | 62.521 | 24.852 | 1.00 | 29.39 |
| ATOM | 1520 | N | TRP | A | 216 | 35.624 | 65.867 | 17.224 | 1.00 | 30.56 |
| ATOM | 1521 | CA | TRP | A | 216 | 35.084 | 66.533 | 16.037 | 1.00 | 31.55 |
| ATOM | 1522 | C | TRP | A | 216 | 34.417 | 65.488 | 15.143 | 1.00 | 32.02 |
| ATOM | 1523 | O | TRP | A | 216 | 34.866 | 64.370 | 15.089 | 1.00 | 31.09 |
| ATOM | 1524 | CB | TRP | A | 216 | 36.202 | 67.133 | 15.221 | 1.00 | 32.03 |
| ATOM | 1525 | CG | TRP | A | 216 | 36.828 | 68.453 | 15.659 | 1.00 | 32.53 |
| ATOM | 1526 | CD1 | TRP | A | 216 | 38.047 | 68.623 | 16.234 | 1.00 | 35.04 |
| ATOM | 1527 | CD2 | TRP | A | 216 | 36.318 | 69.765 | 15.428 | 1.00 | 33.64 |
| ATOM | 1528 | NE1 | TRP | A | 216 | 38.318 | 69.957 | 16.399 | 1.00 | 35.73 |
| ATOM | 1529 | CE2 | TRP | A | 216 | 37.259 | 70.679 | 15.924 | 1.00 | 34.58 |
| ATOM | 1530 | CE3 | TRP | A | 216 | 35.146 | 70.261 | 14.868 | 1.00 | 33.81 |
| ATOM | 1531 | CZ2 | TRP | A | 216 | 37.076 | 72.043 | 15.866 | 1.00 | 34.54 |
| ATOM | 1532 | CZ3 | TRP | A | 216 | 34.967 | 71.608 | 14.811 | 1.00 | 35.06 |
| ATOM | 1533 | CH2 | TRP | A | 216 | 35.930 | 72.491 | 15.310 | 1.00 | 35.33 |
| ATOM | 1534 | N | SER | A | 217 | 33.331 | 65.853 | 14.455 | 1.00 | 33.12 |
| ATOM | 1535 | CA | SER | A | 217 | 32.698 | 64.963 | 13.494 | 1.00 | 33.26 |
| ATOM | 1536 | C | SER | A | 217 | 33.629 | 64.910 | 12.267 | 1.00 | 33.93 |
| ATOM | 1537 | O | SER | A | 217 | 34.552 | 65.709 | 12.145 | 1.00 | 33.61 |
| ATOM | 1538 | CB | SER | A | 217 | 31.289 | 65.465 | 13.119 | 1.00 | 33.02 |
| ATOM | 1539 | OG | SER | A | 217 | 31.362 | 66.694 | 12.380 | 1.00 | 33.81 |
| ATOM | 1540 | N | PRO | A | 218 | 33.463 | 63.936 | 11.381 | 1.00 | 34.85 |
| ATOM | 1541 | CA | PRO | A | 218 | 34.421 | 63.810 | 10.260 | 1.00 | 35.78 |
| ATOM | 1542 | C | PRO | A | 218 | 34.404 | 65.047 | 9.348 | 1.00 | 36.49 |
| ATOM | 1543 | O | PRO | A | 218 | 35.442 | 65.464 | 8.877 | 1.00 | 36.64 |
| ATOM | 1544 | CB | PRO | A | 218 | 34.005 | 62.513 | 9.563 | 1.00 | 36.92 |
| ATOM | 1545 | CG | PRO | A | 218 | 33.048 | 61.787 | 10.573 | 1.00 | 35.44 |
| ATOM | 1546 | CD | PRO | A | 218 | 32.423 | 62.899 | 11.388 | 1.00 | 34.89 |
| ATOM | 1547 | N | ASN | A | 219 | 33.236 | 65.634 | 9.177 | 1.00 | 37.94 |
| ATOM | 1548 | CA | ASN | A | 219 | 33.011 | 66.915 | 8.483 | 1.00 | 39.83 |
| ATOM | 1549 | C | ASN | A | 219 | 33.683 | 68.167 | 9.086 | 1.00 | 40.56 |
| ATOM | 1550 | O | ASN | A | 219 | 33.913 | 69.187 | 8.395 | 1.00 | 40.45 |
| ATOM | 1551 | CB | ASN | A | 219 | 31.519 | 67.268 | 8.647 | 1.00 | 39.50 |
| ATOM | 1552 | CG | ASN | A | 219 | 30.787 | 67.317 | 7.360 | 1.00 | 41.31 |
| ATOM | 1553 | OD1 | ASN | A | 219 | 31.380 | 67.108 | 6.320 | 1.00 | 46.10 |
| ATOM | 1554 | ND2 | ASN | A | 219 | 29.472 | 67.605 | 7.409 | 1.00 | 39.61 |
| ATOM | 1555 | N | GLY | A | 220 | 33.869 | 68.125 | 10.404 | 1.00 | 40.09 |
| ATOM | 1556 | CA | GLY | A | 220 | 34.267 | 69.296 | 11.139 | 1.00 | 40.36 |
| ATOM | 1557 | C | GLY | A | 220 | 33.044 | 70.160 | 11.406 | 1.00 | 40.26 |
| ATOM | 1558 | O | GLY | A | 220 | 33.157 | 71.298 | 11.808 | 1.00 | 40.94 |
| ATOM | 1559 | N | THR | A | 221 | 31.865 | 69.607 | 11.192 | 1.00 | 40.25 |
| ATOM | 1560 | CA | THR | A | 221 | 30.644 | 70.340 | 11.402 | 1.00 | 40.02 |
| ATOM | 1561 | C | THR | A | 221 | 30.442 | 70.461 | 12.899 | 1.00 | 39.77 |
| ATOM | 1562 | O | THR | A | 221 | 30.412 | 71.573 | 13.433 | 1.00 | 39.48 |
| ATOM | 1563 | CB | THR | A | 221 | 29.493 | 69.574 | 10.772 | 1.00 | 40.24 |
| ATOM | 1564 | OG1 | THR | A | 221 | 29.619 | 69.594 | 9.347 | 1.00 | 42.51 |
| ATOM | 1565 | CG2 | THR | A | 221 | 28.189 | 70.258 | 10.991 | 1.00 | 42.08 |
| ATOM | 1566 | N | PHE | A | 222 | 30.337 | 69.292 | 13.548 | 1.00 | 38.47 |
| ATOM | 1567 | CA | PHE | A | 222 | 30.094 | 69.165 | 14.965 | 1.00 | 37.43 |
| ATOM | 1568 | C | PHE | A | 222 | 31.312 | 68.859 | 15.815 | 1.00 | 36.12 |
| ATOM | 1569 | O | PHE | A | 222 | 32.184 | 68.058 | 15.411 | 1.00 | 35.24 |
| ATOM | 1570 | CB | PHE | A | 222 | 29.177 | 67.985 | 15.216 | 1.00 | 37.84 |
| ATOM | 1571 | CG | PHE | A | 222 | 27.878 | 68.040 | 14.490 | 1.00 | 39.38 |
| ATOM | 1572 | CD1 | PHE | A | 222 | 26.830 | 68.768 | 14.996 | 1.00 | 39.66 |
| ATOM | 1573 | CD2 | PHE | A | 222 | 27.693 | 67.310 | 13.320 | 1.00 | 39.54 |
| ATOM | 1574 | CE1 | PHE | A | 222 | 25.605 | 68.804 | 14.331 | 1.00 | 41.91 |
| ATOM | 1575 | CE2 | PHE | A | 222 | 26.488 | 67.332 | 12.661 | 1.00 | 40.33 |
| ATOM | 1576 | CZ | PHE | A | 222 | 25.435 | 68.078 | 13.168 | 1.00 | 40.84 |
| ATOM | 1577 | N | LEU | A | 223 | 31.297 | 69.458 | 17.015 | 1.00 | 34.08 |
| ATOM | 1578 | CA | LEU | A | 223 | 32.253 | 69.207 | 18.093 | 1.00 | 32.28 |
| ATOM | 1579 | C | LEU | A | 223 | 31.474 | 68.612 | 19.231 | 1.00 | 31.26 |
| ATOM | 1580 | O | LEU | A | 223 | 30.575 | 69.264 | 19.804 | 1.00 | 30.76 |
| ATOM | 1581 | CB | LEU | A | 223 | 32.814 | 70.487 | 18.622 | 1.00 | 31.83 |
| ATOM | 1582 | CG | LEU | A | 223 | 34.272 | 70.592 | 19.033 | 1.00 | 31.24 |
| ATOM | 1583 | CD1 | LEU | A | 223 | 34.305 | 71.384 | 20.261 | 1.00 | 27.64 |
| ATOM | 1584 | CD2 | LEU | A | 223 | 35.040 | 69.292 | 19.185 | 1.00 | 30.20 |
| ATOM | 1585 | N | ALA | A | 224 | 31.780 | 67.373 | 19.546 | 1.00 | 29.71 |
| ATOM | 1586 | CA | ALA | A | 224 | 31.167 | 66.759 | 20.667 | 1.00 | 29.39 |
| ATOM | 1587 | C | ALA | A | 224 | 32.211 | 66.839 | 21.766 | 1.00 | 29.58 |
| ATOM | 1588 | O | ALA | A | 224 | 33.414 | 66.879 | 21.481 | 1.00 | 29.87 |
| ATOM | 1589 | CB | ALA | A | 224 | 30.815 | 65.374 | 20.381 | 1.00 | 29.73 |
| ATOM | 1590 | N | TYR | A | 225 | 31.746 | 66.905 | 23.004 | 1.00 | 28.33 |
| ATOM | 1591 | CA | TYR | A | 225 | 32.624 | 66.979 | 24.160 | 1.00 | 28.44 |
| ATOM | 1592 | C | TYR | A | 225 | 31.951 | 66.480 | 25.451 | 1.00 | 27.99 |
| ATOM | 1593 | O | TYR | A | 225 | 30.705 | 66.397 | 25.551 | 1.00 | 27.26 |
| ATOM | 1594 | CB | TYR | A | 225 | 33.106 | 68.386 | 24.375 | 1.00 | 28.46 |
| ATOM | 1595 | CG | TYR | A | 225 | 32.029 | 69.376 | 24.813 | 1.00 | 32.08 |
| ATOM | 1596 | CD1 | TYR | A | 225 | 31.692 | 69.521 | 26.145 | 1.00 | 32.98 |
| ATOM | 1597 | CD2 | TYR | A | 225 | 31.395 | 70.201 | 23.894 | 1.00 | 35.25 |
| ATOM | 1598 | CE1 | TYR | A | 225 | 30.742 | 70.421 | 26.541 | 1.00 | 35.61 |
| ATOM | 1599 | CE2 | TYR | A | 225 | 30.453 | 71.137 | 24.295 | 1.00 | 35.83 |
| ATOM | 1600 | CZ | TYR | A | 225 | 30.125 | 71.227 | 25.616 | 1.00 | 36.19 |
| ATOM | 1601 | OH | TYR | A | 225 | 29.195 | 72.133 | 26.040 | 1.00 | 36.52 |
| ATOM | 1602 | N | ALA | A | 226 | 32.806 | 66.130 | 26.415 | 1.00 | 27.68 |
| ATOM | 1603 | CA | ALA | A | 226 | 32.399 | 65.607 | 27.689 | 1.00 | 26.92 |
| ATOM | 1604 | C | ALA | A | 226 | 32.764 | 66.611 | 28.743 | 1.00 | 27.73 |
| ATOM | 1605 | O | ALA | A | 226 | 33.718 | 67.428 | 28.572 | 1.00 | 28.02 |
| ATOM | 1606 | CB | ALA | A | 226 | 33.052 | 64.303 | 27.961 | 1.00 | 26.51 |
| ATOM | 1607 | N | GLN | A | 227 | 31.979 | 66.590 | 29.821 | 1.00 | 26.98 |
| ATOM | 1608 | CA | GLN | A | 227 | 32.178 | 67.501 | 30.913 | 1.00 | 26.85 |
| ATOM | 1609 | C | GLN | A | 227 | 32.153 | 66.680 | 32.133 | 1.00 | 26.18 |
| ATOM | 1610 | O | GLN | A | 227 | 31.233 | 65.922 | 32.313 | 1.00 | 25.02 |
| ATOM | 1611 | CB | GLN | A | 227 | 31.066 | 68.534 | 31.012 | 1.00 | 27.87 |
| ATOM | 1612 | CG | GLN | A | 227 | 31.129 | 69.423 | 32.276 | 1.00 | 26.85 |
| ATOM | 1613 | CD | GLN | A | 227 | 29.856 | 70.174 | 32.462 | 1.00 | 25.53 |
| ATOM | 1614 | OE1 | GLN | A | 227 | 28.932 | 69.635 | 33.057 | 1.00 | 26.19 |
| ATOM | 1615 | NE2 | GLN | A | 227 | 29.772 | 71.394 | 31.914 | 1.00 | 26.60 |
| ATOM | 1616 | N | PHE | A | 228 | 33.187 | 66.846 | 32.948 | 1.00 | 25.69 |
| ATOM | 1617 | CA | PHE | A | 228 | 33.363 | 66.112 | 34.190 | 1.00 | 26.42 |
| ATOM | 1618 | C | PHE | A | 228 | 33.228 | 67.057 | 35.414 | 1.00 | 27.26 |
| ATOM | 1619 | O | PHE | A | 228 | 33.673 | 68.204 | 35.411 | 1.00 | 27.24 |
| ATOM | 1620 | CB | PHE | A | 228 | 34.722 | 65.395 | 34.210 | 1.00 | 25.89 |
| ATOM | 1621 | CG | PHE | A | 228 | 34.957 | 64.479 | 33.015 | 1.00 | 24.46 |
| ATOM | 1622 | CD1 | PHE | A | 228 | 34.352 | 63.243 | 32.933 | 1.00 | 23.87 |
| ATOM | 1623 | CD2 | PHE | A | 228 | 35.735 | 64.871 | 31.997 | 1.00 | 23.06 |
| ATOM | 1624 | CE1 | PHE | A | 228 | 34.547 | 62.444 | 31.869 | 1.00 | 23.93 |
| ATOM | 1625 | CE2 | PHE | A | 228 | 35.928 | 64.054 | 30.915 | 1.00 | 25.34 |
| ATOM | 1626 | CZ | PHE | A | 228 | 35.322 | 62.852 | 30.852 | 1.00 | 23.94 |
| ATOM | 1627 | N | ASN | A | 229 | 32.568 | 66.551 | 36.434 | 1.00 | 28.68 |
| ATOM | 1628 | CA | ASN | A | 229 | 32.295 | 67.292 | 37.659 | 1.00 | 29.51 |
| ATOM | 1629 | C | ASN | A | 229 | 32.904 | 66.514 | 38.786 | 1.00 | 29.69 |
| ATOM | 1630 | O | ASN | A | 229 | 32.467 | 65.417 | 39.049 | 1.00 | 29.16 |
| ATOM | 1631 | CB | ASN | A | 229 | 30.781 | 67.371 | 37.879 | 1.00 | 30.11 |
| ATOM | 1632 | CG | ASN | A | 229 | 30.409 | 68.340 | 38.981 | 1.00 | 30.80 |
| ATOM | 1633 | OD1 | ASN | A | 229 | 31.127 | 68.432 | 39.990 | 1.00 | 27.42 |
| ATOM | 1634 | ND2 | ASN | A | 229 | 29.280 | 69.083 | 38.778 | 1.00 | 30.54 |
| ATOM | 1635 | N | ASP | A | 230 | 33.923 | 67.085 | 39.435 | 1.00 | 31.03 |
| ATOM | 1636 | CA | ASP | A | 230 | 34.614 | 66.413 | 40.525 | 1.00 | 31.69 |
| ATOM | 1637 | C | ASP | A | 230 | 34.302 | 67.071 | 41.890 | 1.00 | 31.33 |
| ATOM | 1638 | O | ASP | A | 230 | 34.968 | 66.822 | 42.883 | 1.00 | 31.35 |
| ATOM | 1639 | CB | ASP | A | 230 | 36.120 | 66.408 | 40.231 | 1.00 | 31.98 |
| ATOM | 1640 | CG | ASP | A | 230 | 36.512 | 65.329 | 39.212 | 1.00 | 33.87 |
| ATOM | 1641 | OD1 | ASP | A | 230 | 35.938 | 65.329 | 38.087 | 1.00 | 32.91 |
| ATOM | 1642 | OD2 | ASP | A | 230 | 37.361 | 64.439 | 39.458 | 1.00 | 33.90 |
| ATOM | 1643 | N | THR | A | 231 | 33.255 | 67.868 | 41.935 | 1.00 | 31.67 |
| ATOM | 1644 | CA | THR | A | 231 | 32.858 | 68.565 | 43.170 | 1.00 | 32.12 |
| ATOM | 1645 | C | THR | A | 231 | 33.045 | 67.790 | 44.458 | 1.00 | 32.06 |
| ATOM | 1646 | O | THR | A | 231 | 33.673 | 68.307 | 45.387 | 1.00 | 32.58 |
| ATOM | 1647 | CB | THR | A | 231 | 31.416 | 69.087 | 43.087 | 1.00 | 32.16 |
| ATOM | 1648 | OG1 | THR | A | 231 | 31.318 | 70.061 | 42.046 | 1.00 | 31.97 |
| ATOM | 1649 | CG2 | THR | A | 231 | 31.048 | 69.939 | 44.353 | 1.00 | 34.78 |
| ATOM | 1650 | N | GLU | A | 232 | 32.518 | 66.574 | 44.525 | 1.00 | 31.58 |
| ATOM | 1651 | CA | GLU | A | 232 | 32.633 | 65.782 | 45.746 | 1.00 | 31.96 |
| ATOM | 1652 | C | GLU | A | 232 | 33.679 | 64.684 | 45.675 | 1.00 | 30.11 |
| ATOM | 1653 | O | GLU | A | 232 | 33.591 | 63.772 | 46.436 | 1.00 | 29.23 |
| ATOM | 1654 | CB | GLU | A | 232 | 31.273 | 65.070 | 46.060 | 1.00 | 33.29 |
| ATOM | 1655 | CG | GLU | A | 232 | 30.064 | 65.973 | 46.295 | 1.00 | 36.12 |
| ATOM | 1656 | CD | GLU | A | 232 | 28.797 | 65.181 | 46.584 | 1.00 | 42.23 |
| ATOM | 1657 | OE1 | GLU | A | 232 | 28.699 | 64.561 | 47.690 | 1.00 | 46.60 |
| ATOM | 1658 | OE2 | GLU | A | 232 | 27.910 | 65.145 | 45.696 | 1.00 | 43.91 |
| ATOM | 1659 | N | VAL | A | 233 | 34.595 | 64.674 | 44.709 | 1.00 | 28.95 |
| ATOM | 1660 | CA | VAL | A | 233 | 35.585 | 63.588 | 44.698 | 1.00 | 28.12 |
| ATOM | 1661 | C | VAL | A | 233 | 36.618 | 64.015 | 45.717 | 1.00 | 26.69 |
| ATOM | 1662 | O | VAL | A | 233 | 37.020 | 65.149 | 45.677 | 1.00 | 25.94 |
| ATOM | 1663 | CB | VAL | A | 233 | 36.300 | 63.426 | 43.333 | 1.00 | 27.51 |
| ATOM | 1664 | CG1 | VAL | A | 233 | 37.297 | 62.296 | 43.406 | 1.00 | 27.86 |
| ATOM | 1665 | CG2 | VAL | A | 233 | 35.316 | 63.155 | 42.235 | 1.00 | 29.07 |
| ATOM | 1666 | N | PRO | A | 234 | 37.017 | 63.161 | 46.638 | 1.00 | 26.33 |
| ATOM | 1667 | CA | PRO | A | 234 | 38.029 | 63.545 | 47.609 | 1.00 | 26.79 |
| ATOM | 1668 | C | PRO | A | 234 | 39.406 | 63.783 | 46.991 | 1.00 | 27.08 |
| ATOM | 1669 | O | PRO | A | 234 | 39.704 | 63.379 | 45.845 | 1.00 | 26.15 |
| ATOM | 1670 | CB | PRO | A | 234 | 38.062 | 62.358 | 48.564 | 1.00 | 27.74 |
| ATOM | 1671 | CG | PRO | A | 234 | 36.840 | 61.566 | 48.293 | 1.00 | 26.40 |
| ATOM | 1672 | CD | PRO | A | 234 | 36.566 | 61.777 | 46.860 | 1.00 | 27.06 |
| ATOM | 1673 | N | LEU | A | 235 | 40.246 | 64.459 | 47.764 | 1.00 | 26.80 |
| ATOM | 1674 | CA | LEU | A | 235 | 41.537 | 64.873 | 47.289 | 1.00 | 27.73 |
| ATOM | 1675 | C | LEU | A | 235 | 42.615 | 64.040 | 47.830 | 1.00 | 26.54 |
| ATOM | 1676 | O | LEU | A | 235 | 42.621 | 63.857 | 48.996 | 1.00 | 26.56 |
| ATOM | 1677 | CB | LEU | A | 235 | 41.819 | 66.300 | 47.752 | 1.00 | 28.91 |
| ATOM | 1678 | CG | LEU | A | 235 | 40.754 | 67.294 | 47.277 | 1.00 | 31.69 |
| ATOM | 1679 | CD1 | LEU | A | 235 | 40.920 | 68.649 | 47.957 | 1.00 | 32.34 |
| ATOM | 1680 | CD2 | LEU | A | 235 | 40.820 | 67.410 | 45.771 | 1.00 | 32.38 |
| ATOM | 1681 | N | ILE | A | 236 | 43.504 | 63.487 | 47.004 | 1.00 | 25.35 |
| ATOM | 1682 | CA | ILE | A | 236 | 44.723 | 62.929 | 47.574 | 1.00 | 25.28 |
| ATOM | 1683 | C | ILE | A | 236 | 45.668 | 64.117 | 47.741 | 1.00 | 25.50 |
| ATOM | 1684 | O | ILE | A | 236 | 45.717 | 65.003 | 46.891 | 1.00 | 26.26 |
| ATOM | 1685 | CB | ILE | A | 236 | 45.341 | 61.848 | 46.716 | 1.00 | 25.40 |
| ATOM | 1686 | CG1 | ILE | A | 236 | 46.695 | 61.374 | 47.307 | 1.00 | 25.58 |
| ATOM | 1687 | CG2 | ILE | A | 236 | 45.651 | 62.336 | 45.346 | 1.00 | 24.79 |
| ATOM | 1688 | CD1 | ILE | A | 236 | 46.610 | 60.657 | 48.552 | 1.00 | 24.55 |
| ATOM | 1689 | N | GLU | A | 237 | 46.423 | 64.127 | 48.828 | 1.00 | 25.87 |
| ATOM | 1690 | CA | GLU | A | 237 | 47.343 | 65.227 | 49.166 | 1.00 | 26.29 |
| ATOM | 1691 | C | GLU | A | 237 | 48.706 | 64.679 | 49.490 | 1.00 | 26.07 |
| ATOM | 1692 | O | GLU | A | 237 | 48.809 | 63.702 | 50.219 | 1.00 | 26.68 |
| ATOM | 1693 | CB | GLU | A | 237 | 46.834 | 66.023 | 50.364 | 1.00 | 25.50 |
| ATOM | 1694 | CG | GLU | A | 237 | 45.506 | 66.661 | 50.098 | 1.00 | 28.14 |
| ATOM | 1695 | CD | GLU | A | 237 | 45.044 | 67.593 | 51.197 | 1.00 | 30.95 |
| ATOM | 1696 | OE1 | GLU | A | 237 | 45.470 | 67.411 | 52.326 | 1.00 | 36.58 |
| ATOM | 1697 | OE2 | GLU | A | 237 | 44.245 | 68.498 | 50.924 | 1.00 | 33.62 |
| ATOM | 1698 | N | TYR | A | 238 | 49.750 | 65.277 | 48.914 | 1.00 | 25.45 |
| ATOM | 1699 | CA | TYR | A | 238 | 51.112 | 64.861 | 49.221 | 1.00 | 25.12 |
| ATOM | 1700 | C | TYR | A | 238 | 52.014 | 66.056 | 49.002 | 1.00 | 25.07 |
| ATOM | 1701 | O | TYR | A | 238 | 51.636 | 67.011 | 48.319 | 1.00 | 24.71 |
| ATOM | 1702 | CB | TYR | A | 238 | 51.563 | 63.678 | 48.371 | 1.00 | 25.28 |
| ATOM | 1703 | CG | TYR | A | 238 | 51.416 | 63.908 | 46.868 | 1.00 | 24.32 |
| ATOM | 1704 | CD1 | TYR | A | 238 | 52.438 | 64.483 | 46.123 | 1.00 | 24.57 |
| ATOM | 1705 | CD2 | TYR | A | 238 | 50.255 | 63.542 | 46.201 | 1.00 | 26.15 |
| ATOM | 1706 | CE1 | TYR | A | 238 | 52.306 | 64.657 | 44.717 | 1.00 | 25.65 |
| ATOM | 1707 | CE2 | TYR | A | 238 | 50.089 | 63.744 | 44.810 | 1.00 | 25.06 |
| ATOM | 1708 | CZ | TYR | A | 238 | 51.112 | 64.298 | 44.078 | 1.00 | 27.74 |
| ATOM | 1709 | OH | TYR | A | 238 | 50.935 | 64.498 | 42.721 | 1.00 | 29.66 |
| ATOM | 1710 | N | SER | A | 239 | 53.198 | 65.991 | 49.592 | 1.00 | 24.64 |
| ATOM | 1711 | CA | SER | A | 239 | 54.149 | 67.099 | 49.589 | 1.00 | 25.18 |
| ATOM | 1712 | C | SER | A | 239 | 54.986 | 67.085 | 48.329 | 1.00 | 25.42 |
| ATOM | 1713 | O | SER | A | 239 | 55.279 | 66.013 | 47.835 | 1.00 | 25.65 |
| ATOM | 1714 | CB | SER | A | 239 | 55.094 | 66.970 | 50.788 | 1.00 | 23.96 |
| ATOM | 1715 | OG | SER | A | 239 | 54.433 | 67.261 | 51.996 | 1.00 | 26.38 |
| ATOM | 1716 | N | PHE | A | 240 | 55.303 | 68.259 | 47.770 | 1.00 | 25.12 |
| ATOM | 1717 | CA | PHE | A | 240 | 56.235 | 68.334 | 46.636 | 1.00 | 27.19 |
| ATOM | 1718 | C | PHE | A | 240 | 57.338 | 69.297 | 47.116 | 1.00 | 27.23 |
| ATOM | 1719 | O | PHE | A | 240 | 57.045 | 70.421 | 47.479 | 1.00 | 28.10 |
| ATOM | 1720 | CB | PHE | A | 240 | 55.561 | 68.831 | 45.338 | 1.00 | 27.32 |
| ATOM | 1721 | CG | PHE | A | 240 | 56.423 | 68.708 | 44.091 | 1.00 | 26.88 |
| ATOM | 1722 | CD1 | PHE | A | 240 | 56.436 | 67.560 | 43.366 | 1.00 | 29.66 |
| ATOM | 1723 | CD2 | PHE | A | 240 | 57.180 | 69.758 | 43.652 | 1.00 | 26.22 |
| ATOM | 1724 | CE1 | PHE | A | 240 | 57.219 | 67.440 | 42.209 | 1.00 | 31.46 |
| ATOM | 1725 | CE2 | PHE | A | 240 | 57.948 | 69.661 | 42.556 | 1.00 | 29.05 |
| ATOM | 1726 | CZ | PHE | A | 240 | 57.973 | 68.483 | 41.804 | 1.00 | 30.46 |
| ATOM | 1727 | N | TYR | A | 241 | 58.585 | 68.849 | 47.126 | 1.00 | 27.65 |
| ATOM | 1728 | CA | TYR | A | 241 | 59.682 | 69.646 | 47.733 | 1.00 | 27.45 |
| ATOM | 1729 | C | TYR | A | 241 | 60.310 | 70.677 | 46.794 | 1.00 | 27.41 |
| ATOM | 1730 | O | TYR | A | 241 | 60.774 | 71.706 | 47.236 | 1.00 | 27.31 |
| ATOM | 1731 | CB | TYR | A | 241 | 60.713 | 68.702 | 48.345 | 1.00 | 27.05 |
| ATOM | 1732 | CG | TYR | A | 241 | 60.043 | 67.793 | 49.350 | 1.00 | 25.67 |
| ATOM | 1733 | CD1 | TYR | A | 241 | 59.699 | 68.266 | 50.584 | 1.00 | 25.88 |
| ATOM | 1734 | CD2 | TYR | A | 241 | 59.644 | 66.509 | 49.023 | 1.00 | 25.17 |
| ATOM | 1735 | CE1 | TYR | A | 241 | 59.026 | 67.492 | 51.499 | 1.00 | 22.03 |
| ATOM | 1736 | CE2 | TYR | A | 241 | 58.962 | 65.716 | 49.949 | 1.00 | 25.79 |
| ATOM | 1737 | CZ | TYR | A | 241 | 58.653 | 66.227 | 51.190 | 1.00 | 23.56 |
| ATOM | 1738 | OH | TYR | A | 241 | 57.963 | 65.495 | 52.149 | 1.00 | 21.07 |
| ATOM | 1739 | N | SER | A | 242 | 60.253 | 70.413 | 45.497 | 1.00 | 27.48 |
| ATOM | 1740 | CA | SER | A | 242 | 60.798 | 71.303 | 44.478 | 1.00 | 28.37 |
| ATOM | 1741 | C | SER | A | 242 | 62.315 | 71.519 | 44.630 | 1.00 | 28.05 |
| ATOM | 1742 | O | SER | A | 242 | 62.977 | 70.770 | 45.298 | 1.00 | 25.76 |
| ATOM | 1743 | CB | SER | A | 242 | 60.059 | 72.632 | 44.493 | 1.00 | 28.30 |
| ATOM | 1744 | OG | SER | A | 242 | 60.394 | 73.384 | 43.322 | 1.00 | 30.80 |
| ATOM | 1745 | N | ASP | A | 243 | 62.841 | 72.551 | 43.985 | 1.00 | 29.45 |
| ATOM | 1746 | CA | ASP | A | 243 | 64.239 | 72.889 | 44.103 | 1.00 | 30.35 |
| ATOM | 1747 | C | ASP | A | 243 | 64.607 | 73.246 | 45.548 | 1.00 | 30.57 |
| ATOM | 1748 | O | ASP | A | 243 | 63.767 | 73.633 | 46.347 | 1.00 | 29.24 |
| ATOM | 1749 | CB | ASP | A | 243 | 64.525 | 74.099 | 43.236 | 1.00 | 31.66 |
| ATOM | 1750 | CG | ASP | A | 243 | 64.376 | 73.792 | 41.724 | 1.00 | 37.62 |
| ATOM | 1751 | OD1 | ASP | A | 243 | 64.539 | 72.598 | 41.307 | 1.00 | 41.54 |
| ATOM | 1752 | OD2 | ASP | A | 243 | 64.094 | 74.696 | 40.888 | 1.00 | 43.31 |
| ATOM | 1753 | N | GLU | A | 244 | 65.889 | 73.163 | 45.849 | 1.00 | 31.16 |
| ATOM | 1754 | CA | GLU | A | 244 | 66.398 | 73.505 | 47.156 | 1.00 | 32.38 |
| ATOM | 1755 | C | GLU | A | 244 | 65.909 | 74.835 | 47.691 | 1.00 | 32.53 |
| ATOM | 1756 | O | GLU | A | 244 | 65.763 | 75.013 | 48.916 | 1.00 | 29.71 |
| ATOM | 1757 | CB | GLU | A | 244 | 67.895 | 73.640 | 47.058 | 1.00 | 32.97 |
| ATOM | 1758 | CG | GLU | A | 244 | 68.599 | 73.090 | 48.244 | 1.00 | 36.11 |
| ATOM | 1759 | CD | GLU | A | 244 | 70.079 | 73.349 | 48.175 | 1.00 | 38.61 |
| ATOM | 1760 | OE1 | GLU | A | 244 | 70.673 | 72.877 | 47.190 | 1.00 | 37.80 |
| ATOM | 1761 | OE2 | GLU | A | 244 | 70.612 | 74.004 | 49.096 | 1.00 | 38.10 |
| ATOM | 1762 | N | SER | A | 245 | 65.716 | 75.778 | 46.755 | 1.00 | 32.65 |
| ATOM | 1763 | CA | SER | A | 245 | 65.358 | 77.145 | 47.080 | 1.00 | 32.39 |
| ATOM | 1764 | C | SER | A | 245 | 63.979 | 77.307 | 47.670 | 1.00 | 32.11 |
| ATOM | 1765 | O | SER | A | 245 | 63.696 | 78.337 | 48.272 | 1.00 | 32.83 |
| ATOM | 1766 | CB | SER | A | 245 | 65.490 | 78.040 | 45.846 | 1.00 | 32.75 |
| ATOM | 1767 | OG | SER | A | 245 | 64.890 | 77.446 | 44.715 | 1.00 | 34.54 |
| ATOM | 1768 | N | LEU | A | 246 | 63.110 | 76.311 | 47.547 | 1.00 | 31.96 |
| ATOM | 1769 | CA | LEU | A | 246 | 61.750 | 76.508 | 48.086 | 1.00 | 31.53 |
| ATOM | 1770 | C | LEU | A | 246 | 61.838 | 76.325 | 49.579 | 1.00 | 29.60 |
| ATOM | 1771 | O | LEU | A | 246 | 62.186 | 75.250 | 50.040 | 1.00 | 27.72 |
| ATOM | 1772 | CB | LEU | A | 246 | 60.730 | 75.520 | 47.518 | 1.00 | 31.55 |
| ATOM | 1773 | CG | LEU | A | 246 | 59.289 | 75.989 | 47.192 | 1.00 | 35.35 |
| ATOM | 1774 | CD1 | LEU | A | 246 | 58.258 | 74.803 | 47.222 | 1.00 | 36.22 |
| ATOM | 1775 | CD2 | LEU | A | 246 | 58.759 | 77.136 | 47.970 | 1.00 | 34.37 |
| ATOM | 1776 | N | GLN | A | 247 | 61.493 | 77.376 | 50.319 | 1.00 | 28.90 |
| ATOM | 1777 | CA | GLN | A | 247 | 61.577 | 77.346 | 51.762 | 1.00 | 27.73 |
| ATOM | 1778 | C | GLN | A | 247 | 60.535 | 76.405 | 52.413 | 1.00 | 27.30 |
| ATOM | 1779 | O | GLN | A | 247 | 60.857 | 75.551 | 53.263 | 1.00 | 25.43 |
| ATOM | 1780 | CB | GLN | A | 247 | 61.510 | 78.759 | 52.314 | 1.00 | 28.64 |
| ATOM | 1781 | CG | GLN | A | 247 | 61.637 | 78.795 | 53.838 | 1.00 | 28.67 |
| ATOM | 1782 | CD | GLN | A | 247 | 61.930 | 80.174 | 54.399 | 1.00 | 30.01 |
| ATOM | 1783 | OE1 | GLN | A | 247 | 62.833 | 80.347 | 55.276 | 1.00 | 28.72 |
| ATOM | 1784 | NE2 | GLN | A | 247 | 61.177 | 81.152 | 53.940 | 1.00 | 29.07 |
| ATOM | 1785 | N | TYR | A | 248 | 59.305 | 76.502 | 51.946 | 1.00 | 26.79 |
| ATOM | 1786 | CA | TYR | A | 248 | 58.231 | 75.642 | 52.422 | 1.00 | 26.56 |
| ATOM | 1787 | C | TYR | A | 248 | 57.767 | 74.732 | 51.288 | 1.00 | 26.50 |
| ATOM | 1788 | O | TYR | A | 248 | 57.536 | 75.189 | 50.161 | 1.00 | 26.57 |
| ATOM | 1789 | CB | TYR | A | 248 | 57.029 | 76.433 | 52.838 | 1.00 | 26.24 |
| ATOM | 1790 | CG | TYR | A | 248 | 57.240 | 77.264 | 54.079 | 1.00 | 27.06 |
| ATOM | 1791 | CD1 | TYR | A | 248 | 57.980 | 78.450 | 54.039 | 1.00 | 26.07 |
| ATOM | 1792 | CD2 | TYR | A | 248 | 56.685 | 76.875 | 55.285 | 1.00 | 24.74 |
| ATOM | 1793 | CE1 | TYR | A | 248 | 58.175 | 79.206 | 55.193 | 1.00 | 28.15 |
| ATOM | 1794 | CE2 | TYR | A | 248 | 56.884 | 77.617 | 56.448 | 1.00 | 23.55 |
| ATOM | 1795 | CZ | TYR | A | 248 | 57.605 | 78.774 | 56.410 | 1.00 | 25.61 |
| ATOM | 1796 | OH | TYR | A | 248 | 57.744 | 79.500 | 57.583 | 1.00 | 25.29 |
| ATOM | 1797 | N | PRO | A | 249 | 57.664 | 73.457 | 51.583 | 1.00 | 25.69 |
| ATOM | 1798 | CA | PRO | A | 249 | 57.186 | 72.489 | 50.608 | 1.00 | 26.62 |
| ATOM | 1799 | C | PRO | A | 249 | 55.756 | 72.782 | 50.169 | 1.00 | 27.20 |
| ATOM | 1800 | O | PRO | A | 249 | 54.943 | 73.334 | 50.908 | 1.00 | 24.53 |
| ATOM | 1801 | CB | PRO | A | 249 | 57.238 | 71.166 | 51.373 | 1.00 | 27.47 |
| ATOM | 1802 | CG | PRO | A | 249 | 58.249 | 71.400 | 52.431 | 1.00 | 27.65 |
| ATOM | 1803 | CD | PRO | A | 249 | 58.019 | 72.840 | 52.862 | 1.00 | 25.35 |
| ATOM | 1804 | N | LYS | A | 250 | 55.466 | 72.391 | 48.937 | 1.00 | 28.36 |
| ATOM | 1805 | CA | LYS | A | 250 | 54.179 | 72.636 | 48.335 | 1.00 | 30.14 |
| ATOM | 1806 | C | LYS | A | 250 | 53.342 | 71.406 | 48.673 | 1.00 | 29.50 |
| ATOM | 1807 | O | LYS | A | 250 | 53.883 | 70.327 | 48.852 | 1.00 | 31.02 |
| ATOM | 1808 | CB | LYS | A | 250 | 54.407 | 72.812 | 46.805 | 1.00 | 30.66 |
| ATOM | 1809 | CG | LYS | A | 250 | 53.309 | 73.465 | 46.035 | 1.00 | 35.89 |
| ATOM | 1810 | CD | LYS | A | 250 | 53.636 | 73.529 | 44.530 | 1.00 | 40.72 |
| ATOM | 1811 | CE | LYS | A | 250 | 52.387 | 73.724 | 43.659 | 1.00 | 44.34 |
| ATOM | 1812 | NZ | LYS | A | 250 | 52.685 | 73.611 | 42.161 | 1.00 | 45.13 |
| ATOM | 1813 | N | THR | A | 251 | 52.052 | 71.571 | 48.878 | 1.00 | 29.70 |
| ATOM | 1814 | CA | THR | A | 251 | 51.155 | 70.441 | 49.042 | 1.00 | 29.79 |
| ATOM | 1815 | C | THR | A | 251 | 50.389 | 70.285 | 47.711 | 1.00 | 29.85 |
| ATOM | 1816 | O | THR | A | 251 | 49.656 | 71.169 | 47.303 | 1.00 | 29.97 |
| ATOM | 1817 | CB | THR | A | 251 | 50.135 | 70.660 | 50.148 | 1.00 | 29.96 |
| ATOM | 1818 | OG1 | THR | A | 251 | 50.769 | 70.672 | 51.434 | 1.00 | 28.50 |
| ATOM | 1819 | CG2 | THR | A | 251 | 49.157 | 69.445 | 50.230 | 1.00 | 31.41 |
| ATOM | 1820 | N | VAL | A | 252 | 50.571 | 69.175 | 47.025 | 1.00 | 29.44 |
| ATOM | 1821 | CA | VAL | A | 252 | 49.828 | 68.936 | 45.793 | 1.00 | 28.90 |
| ATOM | 1822 | C | VAL | A | 252 | 48.502 | 68.312 | 46.149 | 1.00 | 27.81 |
| ATOM | 1823 | O | VAL | A | 252 | 48.456 | 67.444 | 47.002 | 1.00 | 26.87 |
| ATOM | 1824 | CB | VAL | A | 252 | 50.594 | 67.995 | 44.908 | 1.00 | 29.42 |
| ATOM | 1825 | CG1 | VAL | A | 252 | 49.763 | 67.607 | 43.712 | 1.00 | 31.02 |
| ATOM | 1826 | CG2 | VAL | A | 252 | 51.887 | 68.656 | 44.459 | 1.00 | 30.50 |
| ATOM | 1827 | N | ARG | A | 253 | 47.425 | 68.776 | 45.519 | 1.00 | 27.54 |
| ATOM | 1828 | CA | ARG | A | 253 | 46.066 | 68.291 | 45.804 | 1.00 | 28.17 |
| ATOM | 1829 | C | ARG | A | 253 | 45.369 | 67.924 | 44.528 | 1.00 | 26.49 |
| ATOM | 1830 | O | ARG | A | 253 | 45.202 | 68.747 | 43.684 | 1.00 | 26.20 |
| ATOM | 1831 | CB | ARG | A | 253 | 45.215 | 69.377 | 46.533 | 1.00 | 29.21 |
| ATOM | 1832 | CG | ARG | A | 253 | 45.749 | 69.804 | 47.911 | 1.00 | 31.87 |
| ATOM | 1833 | CD | ARG | A | 253 | 45.055 | 71.048 | 48.487 | 1.00 | 39.81 |
| ATOM | 1834 | NE | ARG | A | 253 | 45.528 | 72.240 | 47.756 | 1.00 | 49.04 |
| ATOM | 1835 | CZ | ARG | A | 253 | 46.583 | 73.015 | 48.126 | 1.00 | 54.08 |
| ATOM | 1836 | NH1 | ARG | A | 253 | 47.260 | 72.775 | 49.254 | 1.00 | 51.93 |
| ATOM | 1837 | NH2 | ARG | A | 253 | 46.938 | 74.053 | 47.375 | 1.00 | 56.78 |
| ATOM | 1838 | N | VAL | A | 254 | 44.897 | 66.701 | 44.414 | 1.00 | 26.67 |
| ATOM | 1839 | CA | VAL | A | 254 | 44.282 | 66.249 | 43.178 | 1.00 | 26.16 |
| ATOM | 1840 | C | VAL | A | 254 | 43.055 | 65.428 | 43.507 | 1.00 | 26.32 |
| ATOM | 1841 | O | VAL | A | 254 | 43.119 | 64.558 | 44.369 | 1.00 | 25.35 |
| ATOM | 1842 | CB | VAL | A | 254 | 45.218 | 65.280 | 42.442 | 1.00 | 27.20 |
| ATOM | 1843 | CG1 | VAL | A | 254 | 44.594 | 64.755 | 41.129 | 1.00 | 26.94 |
| ATOM | 1844 | CG2 | VAL | A | 254 | 46.593 | 65.917 | 42.198 | 1.00 | 27.66 |
| ATOM | 1845 | N | PRO | A | 255 | 41.951 | 65.684 | 42.810 | 1.00 | 24.55 |
| ATOM | 1846 | CA | PRO | A | 255 | 40.759 | 64.856 | 42.931 | 1.00 | 24.42 |
| ATOM | 1847 | C | PRO | A | 255 | 41.057 | 63.482 | 42.339 | 1.00 | 23.94 |
| ATOM | 1848 | O | PRO | A | 255 | 41.366 | 63.386 | 41.161 | 1.00 | 24.49 |
| ATOM | 1849 | CB | PRO | A | 255 | 39.712 | 65.621 | 42.117 | 1.00 | 24.86 |
| ATOM | 1850 | CG | PRO | A | 255 | 40.213 | 66.975 | 41.956 | 1.00 | 24.37 |
| ATOM | 1851 | CD | PRO | A | 255 | 41.743 | 66.781 | 41.867 | 1.00 | 25.52 |
| ATOM | 1852 | N | TYR | A | 256 | 40.923 | 62.466 | 43.167 | 1.00 | 22.02 |
| ATOM | 1853 | CA | TYR | A | 256 | 41.305 | 61.150 | 42.888 | 1.00 | 21.42 |
| ATOM | 1854 | C | TYR | A | 256 | 40.424 | 60.239 | 43.690 | 1.00 | 21.84 |
| ATOM | 1855 | O | TYR | A | 256 | 40.562 | 60.188 | 44.896 | 1.00 | 22.15 |
| ATOM | 1856 | CB | TYR | A | 256 | 42.725 | 60.968 | 43.414 | 1.00 | 20.43 |
| ATOM | 1857 | CG | TYR | A | 256 | 43.336 | 59.599 | 43.166 | 1.00 | 20.95 |
| ATOM | 1858 | CD1 | TYR | A | 256 | 42.920 | 58.486 | 43.867 | 1.00 | 22.41 |
| ATOM | 1859 | CD2 | TYR | A | 256 | 44.371 | 59.440 | 42.283 | 1.00 | 19.15 |
| ATOM | 1860 | CE1 | TYR | A | 256 | 43.500 | 57.259 | 43.687 | 1.00 | 21.46 |
| ATOM | 1861 | CE2 | TYR | A | 256 | 44.948 | 58.223 | 42.083 | 1.00 | 19.11 |
| ATOM | 1862 | CZ | TYR | A | 256 | 44.535 | 57.129 | 42.781 | 1.00 | 21.87 |
| ATOM | 1863 | OH | TYR | A | 256 | 45.127 | 55.875 | 42.546 | 1.00 | 19.77 |
| ATOM | 1864 | N | PRO | A | 257 | 39.560 | 59.467 | 43.031 | 1.00 | 22.36 |
| ATOM | 1865 | CA | PRO | A | 257 | 38.666 | 58.524 | 43.725 | 1.00 | 22.11 |
| ATOM | 1866 | C | PRO | A | 257 | 39.325 | 57.225 | 44.064 | 1.00 | 22.59 |
| ATOM | 1867 | O | PRO | A | 257 | 39.655 | 56.471 | 43.148 | 1.00 | 24.08 |
| ATOM | 1868 | CB | PRO | A | 257 | 37.535 | 58.252 | 42.700 | 1.00 | 22.29 |
| ATOM | 1869 | CG | PRO | A | 257 | 38.102 | 58.730 | 41.316 | 1.00 | 23.05 |
| ATOM | 1870 | CD | PRO | A | 257 | 39.311 | 59.551 | 41.588 | 1.00 | 22.21 |
| ATOM | 1871 | N | LYS | A | 258 | 39.487 | 56.926 | 45.340 | 1.00 | 21.64 |
| ATOM | 1872 | CA | LYS | A | 258 | 40.003 | 55.658 | 45.750 | 1.00 | 21.77 |
| ATOM | 1873 | C | LYS | A | 258 | 38.828 | 54.695 | 45.750 | 1.00 | 22.73 |
| ATOM | 1874 | O | LYS | A | 258 | 37.704 | 55.116 | 45.589 | 1.00 | 22.89 |
| ATOM | 1875 | CB | LYS | A | 258 | 40.737 | 55.745 | 47.096 | 1.00 | 21.30 |
| ATOM | 1876 | CG | LYS | A | 258 | 41.902 | 56.717 | 47.043 | 1.00 | 21.60 |
| ATOM | 1877 | CD | LYS | A | 258 | 42.551 | 57.039 | 48.388 | 1.00 | 22.69 |
| ATOM | 1878 | CE | LYS | A | 258 | 43.967 | 57.664 | 48.222 | 1.00 | 22.08 |
| ATOM | 1879 | NZ | LYS | A | 258 | 45.102 | 56.699 | 48.446 | 1.00 | 19.83 |
| ATOM | 1880 | N | ALA | A | 259 | 39.111 | 53.404 | 45.849 | 1.00 | 23.38 |
| ATOM | 1881 | CA | ALA | A | 259 | 38.100 | 52.365 | 45.759 | 1.00 | 25.01 |
| ATOM | 1882 | C | ALA | A | 259 | 36.915 | 52.595 | 46.691 | 1.00 | 26.15 |
| ATOM | 1883 | O | ALA | A | 259 | 37.087 | 52.705 | 47.920 | 1.00 | 24.93 |
| ATOM | 1884 | CB | ALA | A | 259 | 38.723 | 51.017 | 46.042 | 1.00 | 25.41 |
| ATOM | 1885 | N | GLY | A | 260 | 35.722 | 52.685 | 46.083 | 1.00 | 25.93 |
| ATOM | 1886 | CA | GLY | A | 260 | 34.493 | 52.907 | 46.818 | 1.00 | 26.52 |
| ATOM | 1887 | C | GLY | A | 260 | 34.231 | 54.369 | 47.166 | 1.00 | 27.37 |
| ATOM | 1888 | O | GLY | A | 260 | 33.297 | 54.671 | 47.868 | 1.00 | 28.17 |
| ATOM | 1889 | N | ALA | A | 261 | 35.031 | 55.301 | 46.681 | 1.00 | 27.63 |
| ATOM | 1890 | CA | ALA | A | 261 | 34.779 | 56.694 | 47.042 | 1.00 | 27.82 |
| ATOM | 1891 | C | ALA | A | 261 | 33.853 | 57.342 | 46.022 | 1.00 | 27.48 |
| ATOM | 1892 | O | ALA | A | 261 | 33.516 | 56.715 | 45.067 | 1.00 | 29.08 |
| ATOM | 1893 | CB | ALA | A | 261 | 36.112 | 57.464 | 47.121 | 1.00 | 27.40 |
| ATOM | 1894 | N | VAL | A | 262 | 33.502 | 58.608 | 46.197 | 1.00 | 26.51 |
| ATOM | 1895 | CA | VAL | A | 262 | 32.677 | 59.306 | 45.242 | 1.00 | 26.35 |
| ATOM | 1896 | C | VAL | A | 262 | 33.470 | 59.507 | 43.928 | 1.00 | 27.11 |
| ATOM | 1897 | O | VAL | A | 262 | 34.591 | 60.033 | 43.969 | 1.00 | 27.25 |
| ATOM | 1898 | CB | VAL | A | 262 | 32.216 | 60.670 | 45.871 | 1.00 | 26.18 |
| ATOM | 1899 | CG1 | VAL | A | 262 | 31.510 | 61.532 | 44.897 | 1.00 | 27.60 |
| ATOM | 1900 | CG2 | VAL | A | 262 | 31.290 | 60.445 | 47.100 | 1.00 | 25.69 |
| ATOM | 1901 | N | ASN | A | 263 | 32.911 | 59.063 | 42.783 | 1.00 | 26.63 |
| ATOM | 1902 | CA | ASN | A | 263 | 33.526 | 59.241 | 41.456 | 1.00 | 26.59 |
| ATOM | 1903 | C | ASN | A | 263 | 33.094 | 60.531 | 40.841 | 1.00 | 26.64 |
| ATOM | 1904 | O | ASN | A | 263 | 32.046 | 61.067 | 41.206 | 1.00 | 28.69 |
| ATOM | 1905 | CB | ASN | A | 263 | 33.039 | 58.177 | 40.466 | 1.00 | 26.67 |
| ATOM | 1906 | CG | ASN | A | 263 | 33.876 | 56.899 | 40.462 | 1.00 | 25.47 |
| ATOM | 1907 | OD1 | ASN | A | 263 | 33.447 | 55.858 | 39.889 | 1.00 | 22.78 |
| ATOM | 1908 | ND2 | ASN | A | 263 | 35.032 | 56.935 | 41.104 | 1.00 | 21.04 |
| ATOM | 1909 | N | PRO | A | 264 | 33.823 | 61.022 | 39.855 | 1.00 | 25.42 |
| ATOM | 1910 | CA | PRO | A | 264 | 33.356 | 62.186 | 39.104 | 1.00 | 25.26 |
| ATOM | 1911 | C | PRO | A | 264 | 32.113 | 61.791 | 38.283 | 1.00 | 25.24 |
| ATOM | 1912 | O | PRO | A | 264 | 31.951 | 60.626 | 37.989 | 1.00 | 24.64 |
| ATOM | 1913 | CB | PRO | A | 264 | 34.509 | 62.446 | 38.162 | 1.00 | 25.04 |
| ATOM | 1914 | CG | PRO | A | 264 | 35.114 | 61.031 | 37.971 | 1.00 | 25.43 |
| ATOM | 1915 | CD | PRO | A | 264 | 35.089 | 60.500 | 39.329 | 1.00 | 25.69 |
| ATOM | 1916 | N | THR | A | 265 | 31.265 | 62.742 | 37.936 | 1.00 | 25.73 |
| ATOM | 1917 | CA | THR | A | 265 | 30.099 | 62.489 | 37.086 | 1.00 | 25.58 |
| ATOM | 1918 | C | THR | A | 265 | 30.461 | 63.060 | 35.758 | 1.00 | 25.42 |
| ATOM | 1919 | O | THR | A | 265 | 31.350 | 63.881 | 35.666 | 1.00 | 24.24 |
| ATOM | 1920 | CB | THR | A | 265 | 28.841 | 63.211 | 37.588 | 1.00 | 25.82 |
| ATOM | 1921 | OG1 | THR | A | 265 | 29.161 | 64.580 | 37.928 | 1.00 | 26.18 |
| ATOM | 1922 | CG2 | THR | A | 265 | 28.327 | 62.577 | 38.903 | 1.00 | 27.07 |
| ATOM | 1923 | N | VAL | A | 266 | 29.688 | 62.689 | 34.747 | 1.00 | 25.66 |
| ATOM | 1924 | CA | VAL | A | 266 | 30.023 | 63.031 | 33.391 | 1.00 | 25.11 |
| ATOM | 1925 | C | VAL | A | 266 | 28.770 | 63.344 | 32.634 | 1.00 | 25.81 |
| ATOM | 1926 | O | VAL | A | 266 | 27.747 | 62.675 | 32.823 | 1.00 | 25.44 |
| ATOM | 1927 | CB | VAL | A | 266 | 30.757 | 61.814 | 32.707 | 1.00 | 24.14 |
| ATOM | 1928 | CG1 | VAL | A | 266 | 29.846 | 60.614 | 32.638 | 1.00 | 24.68 |
| ATOM | 1929 | CG2 | VAL | A | 266 | 31.317 | 62.168 | 31.352 | 1.00 | 22.65 |
| ATOM | 1930 | N | LYS | A | 267 | 28.877 | 64.351 | 31.776 | 1.00 | 26.44 |
| ATOM | 1931 | CA | LYS | A | 267 | 27.818 | 64.704 | 30.845 | 1.00 | 28.81 |
| ATOM | 1932 | C | LYS | A | 267 | 28.438 | 64.693 | 29.463 | 1.00 | 29.40 |
| ATOM | 1933 | O | LYS | A | 267 | 29.647 | 64.853 | 29.339 | 1.00 | 29.11 |
| ATOM | 1934 | CB | LYS | A | 267 | 27.266 | 66.090 | 31.154 | 1.00 | 28.26 |
| ATOM | 1935 | CG | LYS | A | 267 | 26.294 | 66.103 | 32.339 | 1.00 | 31.32 |
| ATOM | 1936 | CD | LYS | A | 267 | 25.870 | 67.514 | 32.685 | 1.00 | 32.66 |
| ATOM | 1937 | CE | LYS | A | 267 | 25.259 | 67.592 | 34.075 | 1.00 | 35.71 |
| ATOM | 1938 | NZ | LYS | A | 267 | 24.396 | 68.825 | 34.141 | 1.00 | 36.32 |
| ATOM | 1939 | N | PHE | A | 268 | 27.598 | 64.533 | 28.440 | 1.00 | 30.61 |
| ATOM | 1940 | CA | PHE | A | 268 | 28.025 | 64.571 | 27.034 | 1.00 | 31.41 |
| ATOM | 1941 | C | PHE | A | 268 | 27.235 | 65.605 | 26.204 | 1.00 | 32.03 |
| ATOM | 1942 | O | PHE | A | 268 | 26.005 | 65.608 | 26.229 | 1.00 | 32.82 |
| ATOM | 1943 | CB | PHE | A | 268 | 27.838 | 63.204 | 26.411 | 1.00 | 31.30 |
| ATOM | 1944 | CG | PHE | A | 268 | 28.520 | 63.067 | 25.095 | 1.00 | 31.64 |
| ATOM | 1945 | CD1 | PHE | A | 268 | 29.880 | 62.827 | 25.035 | 1.00 | 33.36 |
| ATOM | 1946 | CD2 | PHE | A | 268 | 27.819 | 63.247 | 23.923 | 1.00 | 31.47 |
| ATOM | 1947 | CE1 | PHE | A | 268 | 30.533 | 62.727 | 23.802 | 1.00 | 32.09 |
| ATOM | 1948 | CE2 | PHE | A | 268 | 28.446 | 63.163 | 22.710 | 1.00 | 32.55 |
| ATOM | 1949 | CZ | PHE | A | 268 | 29.816 | 62.893 | 22.644 | 1.00 | 32.53 |
| ATOM | 1950 | N | PHE | A | 269 | 27.934 | 66.454 | 25.450 | 1.00 | 32.86 |
| ATOM | 1951 | CA | PHE | A | 269 | 27.291 | 67.521 | 24.674 | 1.00 | 32.97 |
| ATOM | 1952 | C | PHE | A | 269 | 27.756 | 67.551 | 23.205 | 1.00 | 32.67 |
| ATOM | 1953 | O | PHE | A | 269 | 28.879 | 67.166 | 22.915 | 1.00 | 32.52 |
| ATOM | 1954 | CB | PHE | A | 269 | 27.638 | 68.890 | 25.279 | 1.00 | 33.14 |
| ATOM | 1955 | CG | PHE | A | 269 | 27.269 | 69.041 | 26.719 | 1.00 | 34.68 |
| ATOM | 1956 | CD1 | PHE | A | 269 | 28.134 | 68.658 | 27.703 | 1.00 | 33.64 |
| ATOM | 1957 | CD2 | PHE | A | 269 | 26.047 | 69.584 | 27.087 | 1.00 | 35.51 |
| ATOM | 1958 | CE1 | PHE | A | 269 | 27.801 | 68.794 | 29.012 | 1.00 | 34.64 |
| ATOM | 1959 | CE2 | PHE | A | 269 | 25.708 | 69.713 | 28.399 | 1.00 | 35.64 |
| ATOM | 1960 | CZ | PHE | A | 269 | 26.588 | 69.304 | 29.372 | 1.00 | 33.01 |
| ATOM | 1961 | N | VAL | A | 270 | 26.915 | 68.049 | 22.294 | 1.00 | 32.38 |
| ATOM | 1962 | CA | VAL | A | 270 | 27.332 | 68.262 | 20.903 | 1.00 | 32.01 |
| ATOM | 1963 | C | VAL | A | 270 | 27.011 | 69.672 | 20.420 | 1.00 | 32.54 |
| ATOM | 1964 | O | VAL | A | 270 | 25.890 | 70.151 | 20.552 | 1.00 | 32.08 |
| ATOM | 1965 | CB | VAL | A | 270 | 26.626 | 67.299 | 19.943 | 1.00 | 32.41 |
| ATOM | 1966 | CG1 | VAL | A | 270 | 27.157 | 67.496 | 18.537 | 1.00 | 30.35 |
| ATOM | 1967 | CG2 | VAL | A | 270 | 26.808 | 65.873 | 20.387 | 1.00 | 31.14 |
| ATOM | 1968 | N | VAL | A | 271 | 27.970 | 70.343 | 19.822 | 1.00 | 33.76 |
| ATOM | 1969 | CA | VAL | A | 271 | 27.705 | 71.683 | 19.309 | 1.00 | 34.90 |
| ATOM | 1970 | C | VAL | A | 271 | 27.898 | 71.808 | 17.809 | 1.00 | 35.24 |
| ATOM | 1971 | O | VAL | A | 271 | 28.907 | 71.374 | 17.296 | 1.00 | 34.81 |
| ATOM | 1972 | CB | VAL | A | 271 | 28.687 | 72.657 | 19.884 | 1.00 | 35.36 |
| ATOM | 1973 | CG1 | VAL | A | 271 | 28.226 | 74.078 | 19.605 | 1.00 | 37.44 |
| ATOM | 1974 | CG2 | VAL | A | 271 | 28.838 | 72.412 | 21.348 | 1.00 | 36.04 |
| ATOM | 1975 | N | ASN | A | 272 | 26.941 | 72.413 | 17.116 | 1.00 | 36.45 |
| ATOM | 1976 | CA | ASN | A | 272 | 27.113 | 72.739 | 15.706 | 1.00 | 37.59 |
| ATOM | 1977 | C | ASN | A | 272 | 27.992 | 73.955 | 15.588 | 1.00 | 38.09 |
| ATOM | 1978 | O | ASN | A | 272 | 27.597 | 75.049 | 15.878 | 1.00 | 37.84 |
| ATOM | 1979 | CB | ASN | A | 272 | 25.803 | 73.040 | 15.001 | 1.00 | 37.83 |
| ATOM | 1980 | CG | ASN | A | 272 | 25.973 | 73.130 | 13.501 | 1.00 | 39.15 |
| ATOM | 1981 | OD1 | ASN | A | 272 | 27.064 | 73.491 | 12.980 | 1.00 | 39.51 |
| ATOM | 1982 | ND2 | ASN | A | 272 | 24.920 | 72.752 | 12.785 | 1.00 | 37.38 |
| ATOM | 1983 | N | THR | A | 273 | 29.189 | 73.691 | 15.127 | 1.00 | 39.64 |
| ATOM | 1984 | CA | THR | A | 273 | 30.254 | 74.616 | 14.914 | 1.00 | 41.12 |
| ATOM | 1985 | C | THR | A | 273 | 30.121 | 75.388 | 13.592 | 1.00 | 42.88 |
| ATOM | 1986 | O | THR | A | 273 | 30.963 | 76.213 | 13.256 | 1.00 | 42.65 |
| ATOM | 1987 | CB | THR | A | 273 | 31.493 | 73.705 | 14.887 | 1.00 | 41.68 |
| ATOM | 1988 | OG1 | THR | A | 273 | 32.322 | 73.920 | 16.053 | 1.00 | 42.46 |
| ATOM | 1989 | CG2 | THR | A | 273 | 32.345 | 73.930 | 13.693 | 1.00 | 40.87 |
| ATOM | 1990 | N | ASP | A | 274 | 29.089 | 75.113 | 12.804 | 1.00 | 44.51 |
| ATOM | 1991 | CA | ASP | A | 274 | 28.940 | 75.871 | 11.565 | 1.00 | 46.51 |
| ATOM | 1992 | C | ASP | A | 274 | 27.976 | 77.009 | 11.802 | 1.00 | 47.68 |
| ATOM | 1993 | O | ASP | A | 274 | 27.891 | 77.940 | 11.012 | 1.00 | 47.48 |
| ATOM | 1994 | CB | ASP | A | 274 | 28.509 | 74.985 | 10.376 | 1.00 | 46.35 |
| ATOM | 1995 | CG | ASP | A | 274 | 29.688 | 74.200 | 9.760 | 1.00 | 46.95 |
| ATOM | 1996 | OD1 | ASP | A | 274 | 30.821 | 74.727 | 9.679 | 1.00 | 47.04 |
| ATOM | 1997 | OD2 | ASP | A | 274 | 29.581 | 73.035 | 9.327 | 1.00 | 50.56 |
| ATOM | 1998 | N | SER | A | 275 | 27.314 | 76.966 | 12.947 | 1.00 | 49.96 |
| ATOM | 1999 | CA | SER | A | 275 | 26.278 | 77.928 | 13.251 | 1.00 | 51.76 |
| ATOM | 2000 | C | SER | A | 275 | 26.606 | 78.783 | 14.462 | 1.00 | 52.74 |
| ATOM | 2001 | O | SER | A | 275 | 25.736 | 79.040 | 15.279 | 1.00 | 52.56 |
| ATOM | 2002 | CB | SER | A | 275 | 24.982 | 77.173 | 13.516 | 1.00 | 52.07 |
| ATOM | 2003 | OG | SER | A | 275 | 25.106 | 76.433 | 14.709 | 1.00 | 52.67 |
| ATOM | 2004 | N | LEU | A | 276 | 27.856 | 79.224 | 14.573 | 1.00 | 54.10 |
| ATOM | 2005 | CA | LEU | A | 276 | 28.275 | 80.037 | 15.706 | 1.00 | 54.88 |
| ATOM | 2006 | C | LEU | A | 276 | 28.193 | 81.494 | 15.334 | 1.00 | 55.90 |
| ATOM | 2007 | O | LEU | A | 276 | 28.238 | 81.830 | 14.163 | 1.00 | 55.21 |
| ATOM | 2008 | CB | LEU | A | 276 | 29.721 | 79.723 | 16.110 | 1.00 | 54.72 |
| ATOM | 2009 | CG | LEU | A | 276 | 30.014 | 78.312 | 16.617 | 1.00 | 53.97 |
| ATOM | 2010 | CD1 | LEU | A | 276 | 31.507 | 78.087 | 16.693 | 1.00 | 53.31 |
| ATOM | 2011 | CD2 | LEU | A | 276 | 29.372 | 78.054 | 17.954 | 1.00 | 52.74 |
| ATOM | 2012 | N | SER | A | 277 | 28.107 | 82.344 | 16.351 | 1.00 | 57.26 |
| ATOM | 2013 | CA | SER | A | 277 | 28.045 | 83.792 | 16.172 | 1.00 | 58.43 |
| ATOM | 2014 | C | SER | A | 277 | 28.727 | 84.521 | 17.328 | 1.00 | 59.40 |
| ATOM | 2015 | O | SER | A | 277 | 28.709 | 84.065 | 18.476 | 1.00 | 59.33 |
| ATOM | 2016 | CB | SER | A | 277 | 26.592 | 84.249 | 16.080 | 1.00 | 58.74 |
| ATOM | 2017 | OG | SER | A | 277 | 26.380 | 85.378 | 16.898 | 1.00 | 58.81 |
| ATOM | 2018 | N | SER | A | 278 | 29.343 | 85.653 | 17.025 | 1.00 | 60.26 |
| ATOM | 2019 | CA | SER | A | 278 | 29.997 | 86.429 | 18.064 | 1.00 | 61.27 |
| ATOM | 2020 | C | SER | A | 278 | 28.981 | 87.052 | 19.033 | 1.00 | 61.57 |
| ATOM | 2021 | O | SER | A | 278 | 29.282 | 87.286 | 20.200 | 1.00 | 62.17 |
| ATOM | 2022 | CB | SER | A | 278 | 30.885 | 87.508 | 17.436 | 1.00 | 61.67 |
| ATOM | 2023 | OG | SER | A | 278 | 30.476 | 87.802 | 16.107 | 1.00 | 61.47 |
| ATOM | 2024 | N | VAL | A | 279 | 27.766 | 87.286 | 18.567 | 1.00 | 61.85 |
| ATOM | 2025 | CA | VAL | A | 279 | 26.763 | 87.921 | 19.412 | 1.00 | 62.09 |
| ATOM | 2026 | C | VAL | A | 279 | 26.070 | 86.953 | 20.337 | 1.00 | 61.70 |
| ATOM | 2027 | O | VAL | A | 279 | 25.911 | 87.210 | 21.532 | 1.00 | 61.70 |
| ATOM | 2028 | CB | VAL | A | 279 | 25.701 | 88.615 | 18.563 | 1.00 | 62.23 |
| ATOM | 2029 | CG1 | VAL | A | 279 | 24.411 | 88.787 | 19.336 | 1.00 | 63.11 |
| ATOM | 2030 | CG2 | VAL | A | 279 | 26.241 | 89.966 | 18.079 | 1.00 | 63.53 |
| ATOM | 2031 | N | THR | A | 280 | 25.635 | 85.845 | 19.770 | 1.00 | 61.15 |
| ATOM | 2032 | CA | THR | A | 280 | 24.894 | 84.869 | 20.531 | 1.00 | 60.92 |
| ATOM | 2033 | C | THR | A | 280 | 25.878 | 83.851 | 21.090 | 1.00 | 60.23 |
| ATOM | 2034 | O | THR | A | 280 | 26.992 | 83.731 | 20.585 | 1.00 | 59.81 |
| ATOM | 2035 | CB | THR | A | 280 | 23.799 | 84.253 | 19.625 | 1.00 | 61.10 |
| ATOM | 2036 | OG1 | THR | A | 280 | 22.619 | 85.069 | 19.702 | 1.00 | 61.10 |
| ATOM | 2037 | CG2 | THR | A | 280 | 23.326 | 82.886 | 20.109 | 1.00 | 61.31 |
| ATOM | 2038 | N | ASN | A | 281 | 25.480 | 83.163 | 22.160 | 1.00 | 59.15 |
| ATOM | 2039 | CA | ASN | A | 281 | 26.326 | 82.144 | 22.774 | 1.00 | 58.27 |
| ATOM | 2040 | C | ASN | A | 281 | 26.082 | 80.806 | 22.138 | 1.00 | 56.62 |
| ATOM | 2041 | O | ASN | A | 281 | 24.981 | 80.520 | 21.646 | 1.00 | 56.51 |
| ATOM | 2042 | CB | ASN | A | 281 | 26.087 | 82.026 | 24.287 | 1.00 | 58.50 |
| ATOM | 2043 | CG | ASN | A | 281 | 26.765 | 83.121 | 25.071 | 1.00 | 60.33 |
| ATOM | 2044 | OD1 | ASN | A | 281 | 27.927 | 83.443 | 24.832 | 1.00 | 62.44 |
| ATOM | 2045 | ND2 | ASN | A | 281 | 26.030 | 83.724 | 26.000 | 1.00 | 64.59 |
| ATOM | 2046 | N | ALA | A | 282 | 27.119 | 79.978 | 22.183 | 1.00 | 54.75 |
| ATOM | 2047 | CA | ALA | A | 282 | 27.089 | 78.657 | 21.591 | 1.00 | 53.17 |
| ATOM | 2048 | C | ALA | A | 282 | 26.007 | 77.776 | 22.194 | 1.00 | 51.89 |
| ATOM | 2049 | O | ALA | A | 282 | 25.768 | 77.757 | 23.392 | 1.00 | 51.73 |
| ATOM | 2050 | CB | ALA | A | 282 | 28.453 | 77.999 | 21.738 | 1.00 | 53.37 |
| ATOM | 2051 | N | THR | A | 283 | 25.374 | 77.026 | 21.321 | 1.00 | 50.24 |
| ATOM | 2052 | CA | THR | A | 283 | 24.312 | 76.134 | 21.668 | 1.00 | 49.16 |
| ATOM | 2053 | C | THR | A | 283 | 24.783 | 74.706 | 21.807 | 1.00 | 47.71 |
| ATOM | 2054 | O | THR | A | 283 | 24.977 | 74.024 | 20.801 | 1.00 | 48.48 |
| ATOM | 2055 | CB | THR | A | 283 | 23.342 | 76.147 | 20.524 | 1.00 | 49.07 |
| ATOM | 2056 | OG1 | THR | A | 283 | 22.738 | 77.439 | 20.434 | 1.00 | 49.95 |
| ATOM | 2057 | CG2 | THR | A | 283 | 22.202 | 75.190 | 20.790 | 1.00 | 50.39 |
| ATOM | 2058 | N | SER | A | 284 | 24.914 | 74.212 | 23.026 | 1.00 | 45.06 |
| ATOM | 2059 | CA | SER | A | 284 | 25.353 | 72.849 | 23.189 | 1.00 | 43.52 |
| ATOM | 2060 | C | SER | A | 284 | 24.132 | 71.988 | 23.452 | 1.00 | 42.27 |
| ATOM | 2061 | O | SER | A | 284 | 23.340 | 72.288 | 24.361 | 1.00 | 41.90 |
| ATOM | 2062 | CB | SER | A | 284 | 26.397 | 72.742 | 24.328 | 1.00 | 44.03 |
| ATOM | 2063 | OG | SER | A | 284 | 27.603 | 73.432 | 23.980 | 1.00 | 43.31 |
| ATOM | 2064 | N | ILE | A | 285 | 23.957 | 70.951 | 22.632 | 1.00 | 40.06 |
| ATOM | 2065 | CA | ILE | A | 285 | 22.898 | 69.982 | 22.833 | 1.00 | 39.55 |
| ATOM | 2066 | C | ILE | A | 285 | 23.412 | 68.790 | 23.658 | 1.00 | 39.06 |
| ATOM | 2067 | O | ILE | A | 285 | 24.395 | 68.138 | 23.293 | 1.00 | 38.89 |
| ATOM | 2068 | CB | ILE | A | 285 | 22.396 | 69.485 | 21.491 | 1.00 | 39.93 |
| ATOM | 2069 | CG1 | ILE | A | 285 | 21.833 | 70.649 | 20.671 | 1.00 | 42.07 |
| ATOM | 2070 | CG2 | ILE | A | 285 | 21.369 | 68.375 | 21.690 | 1.00 | 39.67 |
| ATOM | 2071 | CD1 | ILE | A | 285 | 20.587 | 71.287 | 21.301 | 1.00 | 43.81 |
| ATOM | 2072 | N | GLN | A | 286 | 22.731 | 68.487 | 24.759 | 1.00 | 38.49 |
| ATOM | 2073 | CA | GLN | A | 286 | 23.139 | 67.398 | 25.621 | 1.00 | 37.01 |
| ATOM | 2074 | C | GLN | A | 286 | 22.619 | 66.074 | 25.122 | 1.00 | 36.29 |
| ATOM | 2075 | O | GLN | A | 286 | 21.493 | 65.957 | 24.700 | 1.00 | 35.16 |
| ATOM | 2076 | CB | GLN | A | 286 | 22.630 | 67.616 | 27.055 | 1.00 | 37.50 |
| ATOM | 2077 | CG | GLN | A | 286 | 23.093 | 66.532 | 28.066 | 1.00 | 35.97 |
| ATOM | 2078 | CD | GLN | A | 286 | 22.924 | 66.959 | 29.564 | 1.00 | 38.40 |
| ATOM | 2079 | OE1 | GLN | A | 286 | 22.437 | 68.045 | 29.855 | 1.00 | 37.12 |
| ATOM | 2080 | NE2 | GLN | A | 286 | 23.323 | 66.089 | 30.482 | 1.00 | 31.63 |
| ATOM | 2081 | N | ILE | A | 287 | 23.458 | 65.054 | 25.163 | 1.00 | 36.20 |
| ATOM | 2082 | CA | ILE | A | 287 | 22.945 | 63.720 | 24.944 | 1.00 | 35.42 |
| ATOM | 2083 | C | ILE | A | 287 | 23.014 | 63.082 | 26.304 | 1.00 | 35.21 |
| ATOM | 2084 | O | ILE | A | 287 | 24.099 | 62.964 | 26.873 | 1.00 | 35.34 |
| ATOM | 2085 | CB | ILE | A | 287 | 23.797 | 62.918 | 23.961 | 1.00 | 35.12 |
| ATOM | 2086 | CG1 | ILE | A | 287 | 23.841 | 63.595 | 22.593 | 1.00 | 35.77 |
| ATOM | 2087 | CG2 | ILE | A | 287 | 23.228 | 61.474 | 23.822 | 1.00 | 35.11 |
| ATOM | 2088 | CD1 | ILE | A | 287 | 24.311 | 62.709 | 21.528 | 1.00 | 34.90 |
| ATOM | 2089 | N | THR | A | 288 | 21.892 | 62.639 | 26.831 | 1.00 | 34.92 |
| ATOM | 2090 | CA | THR | A | 288 | 21.922 | 61.970 | 28.127 | 1.00 | 35.17 |
| ATOM | 2091 | C | THR | A | 288 | 22.244 | 60.491 | 27.980 | 1.00 | 35.09 |
| ATOM | 2092 | O | THR | A | 288 | 22.034 | 59.893 | 26.958 | 1.00 | 33.84 |
| ATOM | 2093 | CB | THR | A | 288 | 20.582 | 62.139 | 28.836 | 1.00 | 35.05 |
| ATOM | 2094 | OG1 | THR | A | 288 | 19.536 | 61.783 | 27.943 | 1.00 | 36.03 |
| ATOM | 2095 | CG2 | THR | A | 288 | 20.327 | 63.585 | 29.135 | 1.00 | 36.30 |
| ATOM | 2096 | N | ALA | A | 289 | 22.748 | 59.902 | 29.040 | 1.00 | 35.95 |
| ATOM | 2097 | CA | ALA | A | 289 | 23.099 | 58.512 | 29.022 | 1.00 | 36.76 |
| ATOM | 2098 | C | ALA | A | 289 | 21.820 | 57.724 | 29.139 | 1.00 | 37.41 |
| ATOM | 2099 | O | ALA | A | 289 | 20.813 | 58.230 | 29.610 | 1.00 | 38.32 |
| ATOM | 2100 | CB | ALA | A | 289 | 24.016 | 58.200 | 30.189 | 1.00 | 36.36 |
| ATOM | 2101 | N | PRO | A | 290 | 21.862 | 56.485 | 28.687 | 1.00 | 37.99 |
| ATOM | 2102 | CA | PRO | A | 290 | 20.718 | 55.579 | 28.748 | 1.00 | 38.30 |
| ATOM | 2103 | C | PRO | A | 290 | 20.152 | 55.447 | 30.159 | 1.00 | 38.42 |
| ATOM | 2104 | O | PRO | A | 290 | 20.919 | 55.514 | 31.149 | 1.00 | 37.42 |
| ATOM | 2105 | CB | PRO | A | 290 | 21.320 | 54.224 | 28.386 | 1.00 | 38.47 |
| ATOM | 2106 | CG | PRO | A | 290 | 22.594 | 54.489 | 27.757 | 1.00 | 38.29 |
| ATOM | 2107 | CD | PRO | A | 290 | 23.028 | 55.879 | 28.045 | 1.00 | 38.09 |
| ATOM | 2108 | N | ALA | A | 291 | 18.844 | 55.200 | 30.229 | 1.00 | 37.43 |
| ATOM | 2109 | CA | ALA | A | 291 | 18.144 | 55.033 | 31.497 | 1.00 | 37.84 |
| ATOM | 2110 | C | ALA | A | 291 | 18.765 | 53.931 | 32.357 | 1.00 | 37.19 |
| ATOM | 2111 | O | ALA | A | 291 | 18.768 | 54.002 | 33.587 | 1.00 | 36.25 |
| ATOM | 2112 | CB | ALA | A | 291 | 16.661 | 54.713 | 31.231 | 1.00 | 37.87 |
| ATOM | 2113 | N | SER | A | 292 | 19.261 | 52.896 | 31.704 | 1.00 | 36.91 |
| ATOM | 2114 | CA | SER | A | 292 | 19.833 | 51.789 | 32.426 | 1.00 | 37.70 |
| ATOM | 2115 | C | SER | A | 292 | 21.222 | 52.115 | 33.015 | 1.00 | 37.65 |
| ATOM | 2116 | O | SER | A | 292 | 21.748 | 51.355 | 33.823 | 1.00 | 38.56 |
| ATOM | 2117 | CB | SER | A | 292 | 19.905 | 50.573 | 31.554 | 1.00 | 37.31 |
| ATOM | 2118 | OG | SER | A | 292 | 20.880 | 50.771 | 30.553 | 1.00 | 39.95 |
| ATOM | 2119 | N | MET | A | 293 | 21.783 | 53.250 | 32.630 | 1.00 | 37.04 |
| ATOM | 2120 | CA | MET | A | 293 | 23.026 | 53.714 | 33.210 | 1.00 | 37.24 |
| ATOM | 2121 | C | MET | A | 293 | 22.661 | 54.752 | 34.236 | 1.00 | 37.75 |
| ATOM | 2122 | O | MET | A | 293 | 23.286 | 54.865 | 35.298 | 1.00 | 37.19 |
| ATOM | 2123 | CB | MET | A | 293 | 23.936 | 54.325 | 32.142 | 1.00 | 36.55 |
| ATOM | 2124 | CG | MET | A | 293 | 24.469 | 53.296 | 31.175 | 1.00 | 34.83 |
| ATOM | 2125 | SD | MET | A | 293 | 25.771 | 52.417 | 31.979 | 1.00 | 34.30 |
| ATOM | 2126 | CE | MET | A | 293 | 25.702 | 50.765 | 31.245 | 1.00 | 36.98 |
| ATOM | 2127 | N | LEU | A | 294 | 21.591 | 55.469 | 33.937 | 1.00 | 38.01 |
| ATOM | 2128 | CA | LEU | A | 294 | 21.137 | 56.545 | 34.797 | 1.00 | 38.65 |
| ATOM | 2129 | C | LEU | A | 294 | 20.714 | 56.084 | 36.173 | 1.00 | 38.03 |
| ATOM | 2130 | O | LEU | A | 294 | 20.688 | 56.878 | 37.093 | 1.00 | 37.83 |
| ATOM | 2131 | CB | LEU | A | 294 | 20.009 | 57.315 | 34.105 | 1.00 | 39.32 |
| ATOM | 2132 | CG | LEU | A | 294 | 20.330 | 58.728 | 33.640 | 1.00 | 40.15 |
| ATOM | 2133 | CD1 | LEU | A | 294 | 21.790 | 58.848 | 33.338 | 1.00 | 40.48 |
| ATOM | 2134 | CD2 | LEU | A | 294 | 19.494 | 59.123 | 32.449 | 1.00 | 41.68 |
| ATOM | 2135 | N | ILE | A | 295 | 20.441 | 54.795 | 36.322 | 1.00 | 38.36 |
| ATOM | 2136 | CA | ILE | A | 295 | 20.032 | 54.214 | 37.615 | 1.00 | 39.03 |
| ATOM | 2137 | C | ILE | A | 295 | 21.062 | 54.413 | 38.743 | 1.00 | 37.93 |
| ATOM | 2138 | O | ILE | A | 295 | 20.705 | 54.505 | 39.912 | 1.00 | 37.90 |
| ATOM | 2139 | CB | ILE | A | 295 | 19.834 | 52.680 | 37.472 | 1.00 | 39.30 |
| ATOM | 2140 | CG1 | ILE | A | 295 | 18.553 | 52.306 | 36.750 | 1.00 | 43.56 |
| ATOM | 2141 | CG2 | ILE | A | 295 | 19.642 | 52.060 | 38.802 | 1.00 | 42.21 |
| ATOM | 2142 | CD1 | ILE | A | 295 | 18.528 | 50.744 | 36.416 | 1.00 | 47.46 |
| ATOM | 2143 | N | GLY | A | 296 | 22.345 | 54.408 | 38.386 | 1.00 | 36.37 |
| ATOM | 2144 | CA | GLY | A | 296 | 23.434 | 54.437 | 39.362 | 1.00 | 35.27 |
| ATOM | 2145 | C | GLY | A | 296 | 24.692 | 55.134 | 38.825 | 1.00 | 33.95 |
| ATOM | 2146 | O | GLY | A | 296 | 24.623 | 55.882 | 37.858 | 1.00 | 33.25 |
| ATOM | 2147 | N | ASP | A | 297 | 25.820 | 54.936 | 39.498 | 1.00 | 32.12 |
| ATOM | 2148 | CA | ASP | A | 297 | 27.074 | 55.508 | 39.067 | 1.00 | 30.86 |
| ATOM | 2149 | C | ASP | A | 297 | 27.442 | 54.812 | 37.763 | 1.00 | 29.01 |
| ATOM | 2150 | O | ASP | A | 297 | 27.265 | 53.631 | 37.670 | 1.00 | 25.96 |
| ATOM | 2151 | CB | ASP | A | 297 | 28.144 | 55.203 | 40.111 | 1.00 | 31.53 |
| ATOM | 2152 | CG | ASP | A | 297 | 28.157 | 56.201 | 41.278 | 1.00 | 32.76 |
| ATOM | 2153 | OD1 | ASP | A | 297 | 27.575 | 57.306 | 41.204 | 1.00 | 37.01 |
| ATOM | 2154 | OD2 | ASP | A | 297 | 28.762 | 55.951 | 42.305 | 1.00 | 34.16 |
| ATOM | 2155 | N | HIS | A | 298 | 27.969 | 55.541 | 36.779 | 1.00 | 28.26 |
| ATOM | 2156 | CA | HIS | A | 298 | 28.365 | 54.930 | 35.500 | 1.00 | 28.24 |
| ATOM | 2157 | C | HIS | A | 298 | 29.514 | 55.729 | 34.923 | 1.00 | 27.63 |
| ATOM | 2158 | O | HIS | A | 298 | 29.873 | 56.742 | 35.468 | 1.00 | 28.58 |
| ATOM | 2159 | CB | HIS | A | 298 | 27.179 | 54.959 | 34.509 | 1.00 | 28.44 |
| ATOM | 2160 | CG | HIS | A | 298 | 26.509 | 56.293 | 34.465 | 1.00 | 27.81 |
| ATOM | 2161 | ND1 | HIS | A | 298 | 26.917 | 57.296 | 33.617 | 1.00 | 24.43 |
| ATOM | 2162 | CD2 | HIS | A | 298 | 25.492 | 56.805 | 35.197 | 1.00 | 25.10 |
| ATOM | 2163 | CE1 | HIS | A | 298 | 26.214 | 58.387 | 33.858 | 1.00 | 25.84 |
| ATOM | 2164 | NE2 | HIS | A | 298 | 25.318 | 58.108 | 34.792 | 1.00 | 26.84 |
| ATOM | 2165 | N | TYR | A | 299 | 30.090 | 55.266 | 33.825 | 1.00 | 27.33 |
| ATOM | 2166 | CA | TYR | A | 299 | 31.157 | 55.984 | 33.129 | 1.00 | 27.46 |
| ATOM | 2167 | C | TYR | A | 299 | 30.819 | 56.134 | 31.652 | 1.00 | 28.16 |
| ATOM | 2168 | O | TYR | A | 299 | 30.091 | 55.308 | 31.084 | 1.00 | 28.79 |
| ATOM | 2169 | CB | TYR | A | 299 | 32.469 | 55.170 | 33.160 | 1.00 | 27.18 |
| ATOM | 2170 | CG | TYR | A | 299 | 32.962 | 54.739 | 34.517 | 1.00 | 24.61 |
| ATOM | 2171 | CD1 | TYR | A | 299 | 33.572 | 55.650 | 35.393 | 1.00 | 25.18 |
| ATOM | 2172 | CD2 | TYR | A | 299 | 32.903 | 53.429 | 34.892 | 1.00 | 21.82 |
| ATOM | 2173 | CE1 | TYR | A | 299 | 34.021 | 55.260 | 36.626 | 1.00 | 23.54 |
| ATOM | 2174 | CE2 | TYR | A | 299 | 33.373 | 53.024 | 36.140 | 1.00 | 27.88 |
| ATOM | 2175 | CZ | TYR | A | 299 | 33.915 | 53.950 | 37.006 | 1.00 | 23.90 |
| ATOM | 2176 | OH | TYR | A | 299 | 34.396 | 53.530 | 38.227 | 1.00 | 25.14 |
| ATOM | 2177 | N | LEU | A | 300 | 31.344 | 57.185 | 31.037 | 1.00 | 28.85 |
| ATOM | 2178 | CA | LEU | A | 300 | 31.366 | 57.304 | 29.588 | 1.00 | 29.34 |
| ATOM | 2179 | C | LEU | A | 300 | 32.715 | 56.697 | 29.201 | 1.00 | 29.69 |
| ATOM | 2180 | O | LEU | A | 300 | 33.744 | 57.252 | 29.571 | 1.00 | 30.77 |
| ATOM | 2181 | CB | LEU | A | 300 | 31.334 | 58.764 | 29.170 | 1.00 | 29.67 |
| ATOM | 2182 | CG | LEU | A | 300 | 31.261 | 59.019 | 27.651 | 1.00 | 30.84 |
| ATOM | 2183 | CD1 | LEU | A | 300 | 31.833 | 60.334 | 27.339 | 1.00 | 30.08 |
| ATOM | 2184 | CD2 | LEU | A | 300 | 32.026 | 58.008 | 26.912 | 1.00 | 34.39 |
| ATOM | 2185 | N | CYS | A | 301 | 32.726 | 55.595 | 28.458 | 1.00 | 30.05 |
| ATOM | 2186 | CA | CYS | A | 301 | 33.965 | 54.803 | 28.252 | 1.00 | 29.99 |
| ATOM | 2187 | C | CYS | A | 301 | 34.539 | 55.003 | 26.783 | 1.00 | 32.42 |
| ATOM | 2188 | O | CYS | A | 301 | 35.767 | 54.889 | 26.564 | 1.00 | 32.99 |
| ATOM | 2189 | CB | CYS | A | 301 | 33.772 | 53.270 | 28.856 | 1.00 | 30.22 |
| ATOM | 2190 | SG | CYS | A | 301 | 34.468 | 52.763 | 30.633 | 1.00 | 21.35 |
| ATOM | 2191 | N | ASP | A | 302 | 33.720 | 55.415 | 25.797 | 1.00 | 33.24 |
| ATOM | 2192 | CA | ASP | A | 302 | 34.165 | 55.509 | 24.374 | 1.00 | 34.53 |
| ATOM | 2193 | C | ASP | A | 302 | 33.171 | 56.299 | 23.480 | 1.00 | 33.63 |
| ATOM | 2194 | O | ASP | A | 302 | 31.954 | 56.160 | 23.599 | 1.00 | 33.45 |
| ATOM | 2195 | CB | ASP | A | 302 | 34.333 | 54.106 | 23.777 | 1.00 | 36.11 |
| ATOM | 2196 | CG | ASP | A | 302 | 34.995 | 54.115 | 22.399 | 1.00 | 39.99 |
| ATOM | 2197 | OD1 | ASP | A | 302 | 36.254 | 54.027 | 22.318 | 1.00 | 48.32 |
| ATOM | 2198 | OD2 | ASP | A | 302 | 34.356 | 54.180 | 21.330 | 1.00 | 46.68 |
| ATOM | 2199 | N | VAL | A | 303 | 33.704 | 57.144 | 22.617 | 1.00 | 31.84 |
| ATOM | 2200 | CA | VAL | A | 303 | 32.921 | 58.030 | 21.783 | 1.00 | 31.33 |
| ATOM | 2201 | C | VAL | A | 303 | 33.383 | 57.807 | 20.355 | 1.00 | 31.55 |
| ATOM | 2202 | O | VAL | A | 303 | 34.568 | 57.929 | 20.070 | 1.00 | 29.81 |
| ATOM | 2203 | CB | VAL | A | 303 | 33.142 | 59.517 | 22.167 | 1.00 | 30.97 |
| ATOM | 2204 | CG1 | VAL | A | 303 | 32.554 | 60.471 | 21.124 | 1.00 | 31.12 |
| ATOM | 2205 | CG2 | VAL | A | 303 | 32.535 | 59.826 | 23.528 | 1.00 | 31.39 |
| ATOM | 2206 | N | THR | A | 304 | 32.473 | 57.467 | 19.454 | 1.00 | 30.98 |
| ATOM | 2207 | CA | THR | A | 304 | 32.922 | 57.241 | 18.060 | 1.00 | 32.60 |
| ATOM | 2208 | C | THR | A | 304 | 31.983 | 57.831 | 17.012 | 1.00 | 32.56 |
| ATOM | 2209 | O | THR | A | 304 | 30.812 | 57.481 | 16.980 | 1.00 | 32.98 |
| ATOM | 2210 | CB | THR | A | 304 | 33.073 | 55.765 | 17.793 | 1.00 | 32.09 |
| ATOM | 2211 | OG1 | THR | A | 304 | 34.098 | 55.200 | 18.636 | 1.00 | 36.13 |
| ATOM | 2212 | CG2 | THR | A | 304 | 33.582 | 55.526 | 16.426 | 1.00 | 32.81 |
| ATOM | 2213 | N | TRP | A | 305 | 32.472 | 58.715 | 16.152 | 1.00 | 32.85 |
| ATOM | 2214 | CA | TRP | A | 305 | 31.617 | 59.188 | 15.062 | 1.00 | 33.54 |
| ATOM | 2215 | C | TRP | A | 305 | 31.488 | 58.066 | 13.993 | 1.00 | 33.99 |
| ATOM | 2216 | O | TRP | A | 305 | 32.489 | 57.644 | 13.436 | 1.00 | 35.39 |
| ATOM | 2217 | CB | TRP | A | 305 | 32.168 | 60.471 | 14.449 | 1.00 | 33.79 |
| ATOM | 2218 | CG | TRP | A | 305 | 31.974 | 61.622 | 15.291 | 1.00 | 33.47 |
| ATOM | 2219 | CD1 | TRP | A | 305 | 32.839 | 62.105 | 16.213 | 1.00 | 32.15 |
| ATOM | 2220 | CD2 | TRP | A | 305 | 30.814 | 62.454 | 15.353 | 1.00 | 33.18 |
| ATOM | 2221 | NE1 | TRP | A | 305 | 32.297 | 63.195 | 16.830 | 1.00 | 31.30 |
| ATOM | 2222 | CE2 | TRP | A | 305 | 31.051 | 63.427 | 16.326 | 1.00 | 31.04 |
| ATOM | 2223 | CE3 | TRP | A | 305 | 29.612 | 62.491 | 14.660 | 1.00 | 32.12 |
| ATOM | 2224 | CZ2 | TRP | A | 305 | 30.128 | 64.404 | 16.643 | 1.00 | 32.78 |
| ATOM | 2225 | CZ3 | TRP | A | 305 | 28.706 | 63.458 | 14.973 | 1.00 | 35.35 |
| ATOM | 2226 | CH2 | TRP | A | 305 | 28.956 | 64.399 | 15.960 | 1.00 | 33.19 |
| ATOM | 2227 | N | ALA | A | 306 | 30.276 | 57.572 | 13.753 | 1.00 | 33.77 |
| ATOM | 2228 | CA | ALA | A | 306 | 30.031 | 56.545 | 12.756 | 1.00 | 33.97 |
| ATOM | 2229 | C | ALA | A | 306 | 29.846 | 57.188 | 11.412 | 1.00 | 34.54 |
| ATOM | 2230 | O | ALA | A | 306 | 30.404 | 56.726 | 10.437 | 1.00 | 35.03 |
| ATOM | 2231 | CB | ALA | A | 306 | 28.815 | 55.731 | 13.085 | 1.00 | 34.43 |
| ATOM | 2232 | N | THR | A | 307 | 29.059 | 58.244 | 11.342 | 1.00 | 34.83 |
| ATOM | 2233 | CA | THR | A | 307 | 28.962 | 58.998 | 10.104 | 1.00 | 35.28 |
| ATOM | 2234 | C | THR | A | 307 | 28.993 | 60.468 | 10.442 | 1.00 | 36.71 |
| ATOM | 2235 | O | THR | A | 307 | 29.299 | 60.835 | 11.579 | 1.00 | 38.20 |
| ATOM | 2236 | CB | THR | A | 307 | 27.666 | 58.689 | 9.381 | 1.00 | 35.21 |
| ATOM | 2237 | OG1 | THR | A | 307 | 26.562 | 59.189 | 10.150 | 1.00 | 31.75 |
| ATOM | 2238 | CG2 | THR | A | 307 | 27.441 | 57.184 | 9.275 | 1.00 | 34.16 |
| ATOM | 2239 | N | GLN | A | 308 | 28.675 | 61.303 | 9.456 | 1.00 | 36.94 |
| ATOM | 2240 | CA | GLN | A | 308 | 28.595 | 62.746 | 9.610 | 1.00 | 36.92 |
| ATOM | 2241 | C | GLN | A | 308 | 27.472 | 63.119 | 10.560 | 1.00 | 37.03 |
| ATOM | 2242 | O | GLN | A | 308 | 27.420 | 64.244 | 11.084 | 1.00 | 37.35 |
| ATOM | 2243 | CB | GLN | A | 308 | 28.337 | 63.443 | 8.245 | 1.00 | 37.54 |
| ATOM | 2244 | CG | GLN | A | 308 | 29.417 | 63.194 | 7.140 | 1.00 | 38.17 |
| ATOM | 2245 | CD | GLN | A | 308 | 30.813 | 63.689 | 7.546 | 1.00 | 42.65 |
| ATOM | 2246 | OE1 | GLN | A | 308 | 30.954 | 64.550 | 8.441 | 1.00 | 43.08 |
| ATOM | 2247 | NE2 | GLN | A | 308 | 31.850 | 63.123 | 6.918 | 1.00 | 40.49 |
| ATOM | 2248 | N | GLU | A | 309 | 26.567 | 62.183 | 10.805 | 1.00 | 36.71 |
| ATOM | 2249 | CA | GLU | A | 309 | 25.377 | 62.488 | 11.575 | 1.00 | 35.50 |
| ATOM | 2250 | C | GLU | A | 309 | 25.050 | 61.421 | 12.602 | 1.00 | 34.91 |
| ATOM | 2251 | O | GLU | A | 309 | 23.963 | 61.416 | 13.167 | 1.00 | 35.07 |
| ATOM | 2252 | CB | GLU | A | 309 | 24.191 | 62.676 | 10.594 | 1.00 | 36.50 |
| ATOM | 2253 | CG | GLU | A | 309 | 24.558 | 63.640 | 9.472 | 1.00 | 36.43 |
| ATOM | 2254 | CD | GLU | A | 309 | 23.413 | 64.366 | 8.766 | 1.00 | 39.46 |
| ATOM | 2255 | OE1 | GLU | A | 309 | 22.199 | 64.156 | 9.081 | 1.00 | 34.63 |
| ATOM | 2256 | OE2 | GLU | A | 309 | 23.785 | 65.180 | 7.854 | 1.00 | 39.80 |
| ATOM | 2257 | N | ARG | A | 310 | 25.974 | 60.508 | 12.858 | 1.00 | 33.71 |
| ATOM | 2258 | CA | ARG | A | 310 | 25.690 | 59.428 | 13.769 | 1.00 | 33.41 |
| ATOM | 2259 | C | ARG | A | 310 | 26.826 | 59.287 | 14.765 | 1.00 | 33.15 |
| ATOM | 2260 | O | ARG | A | 310 | 27.982 | 59.203 | 14.356 | 1.00 | 32.29 |
| ATOM | 2261 | CB | ARG | A | 310 | 25.538 | 58.154 | 12.972 | 1.00 | 33.68 |
| ATOM | 2262 | CG | ARG | A | 310 | 25.274 | 56.893 | 13.777 | 1.00 | 36.39 |
| ATOM | 2263 | CD | ARG | A | 310 | 24.660 | 55.779 | 12.922 | 1.00 | 39.54 |
| ATOM | 2264 | NE | ARG | A | 310 | 23.250 | 55.583 | 13.231 | 1.00 | 43.09 |
| ATOM | 2265 | CZ | ARG | A | 310 | 22.353 | 55.034 | 12.434 | 1.00 | 46.21 |
| ATOM | 2266 | NH1 | ARG | A | 310 | 22.686 | 54.636 | 11.224 | 1.00 | 48.82 |
| ATOM | 2267 | NH2 | ARG | A | 310 | 21.089 | 54.925 | 12.845 | 1.00 | 47.65 |
| ATOM | 2268 | N | ILE | A | 311 | 26.494 | 59.243 | 16.055 | 1.00 | 33.31 |
| ATOM | 2269 | CA | ILE | A | 311 | 27.511 | 59.117 | 17.115 | 1.00 | 34.40 |
| ATOM | 2270 | C | ILE | A | 311 | 27.264 | 57.868 | 17.923 | 1.00 | 33.36 |
| ATOM | 2271 | O | ILE | A | 311 | 26.146 | 57.573 | 18.313 | 1.00 | 33.46 |
| ATOM | 2272 | CB | ILE | A | 311 | 27.532 | 60.338 | 18.101 | 1.00 | 34.77 |
| ATOM | 2273 | CG1 | ILE | A | 311 | 27.489 | 61.662 | 17.375 | 1.00 | 37.84 |
| ATOM | 2274 | CG2 | ILE | A | 311 | 28.825 | 60.388 | 18.907 | 1.00 | 36.40 |
| ATOM | 2275 | CD1 | ILE | A | 311 | 26.952 | 62.833 | 18.302 | 1.00 | 39.22 |
| ATOM | 2276 | N | SER | A | 312 | 28.327 | 57.140 | 18.197 | 1.00 | 32.30 |
| ATOM | 2277 | CA | SER | A | 312 | 28.213 | 55.957 | 19.042 | 1.00 | 32.09 |
| ATOM | 2278 | C | SER | A | 312 | 28.804 | 56.335 | 20.395 | 1.00 | 31.13 |
| ATOM | 2279 | O | SER | A | 312 | 29.871 | 56.919 | 20.425 | 1.00 | 30.12 |
| ATOM | 2280 | CB | SER | A | 312 | 29.020 | 54.844 | 18.416 | 1.00 | 32.17 |
| ATOM | 2281 | OG | SER | A | 312 | 28.961 | 53.709 | 19.195 | 1.00 | 32.79 |
| ATOM | 2282 | N | LEU | A | 313 | 28.095 | 56.053 | 21.485 | 1.00 | 30.37 |
| ATOM | 2283 | CA | LEU | A | 313 | 28.612 | 56.297 | 22.837 | 1.00 | 31.25 |
| ATOM | 2284 | C | LEU | A | 313 | 28.550 | 55.006 | 23.623 | 1.00 | 30.83 |
| ATOM | 2285 | O | LEU | A | 313 | 27.451 | 54.441 | 23.718 | 1.00 | 31.63 |
| ATOM | 2286 | CB | LEU | A | 313 | 27.777 | 57.335 | 23.584 | 1.00 | 30.44 |
| ATOM | 2287 | CG | LEU | A | 313 | 27.584 | 58.683 | 22.918 | 1.00 | 32.85 |
| ATOM | 2288 | CD1 | LEU | A | 313 | 26.682 | 59.541 | 23.773 | 1.00 | 32.34 |
| ATOM | 2289 | CD2 | LEU | A | 313 | 28.908 | 59.377 | 22.685 | 1.00 | 34.95 |
| ATOM | 2290 | N | GLN | A | 314 | 29.686 | 54.520 | 24.148 | 1.00 | 30.07 |
| ATOM | 2291 | CA | GLN | A | 314 | 29.689 | 53.350 | 25.021 | 1.00 | 30.67 |
| ATOM | 2292 | C | GLN | A | 314 | 29.751 | 53.818 | 26.476 | 1.00 | 28.79 |
| ATOM | 2293 | O | GLN | A | 314 | 30.629 | 54.604 | 26.841 | 1.00 | 28.37 |
| ATOM | 2294 | CB | GLN | A | 314 | 30.871 | 52.388 | 24.791 | 1.00 | 31.25 |
| ATOM | 2295 | CG | GLN | A | 314 | 30.608 | 51.259 | 23.849 | 1.00 | 37.45 |
| ATOM | 2296 | CD | GLN | A | 314 | 31.550 | 50.026 | 24.008 | 1.00 | 39.54 |
| ATOM | 2297 | OE1 | GLN | A | 314 | 31.070 | 48.894 | 24.133 | 1.00 | 40.38 |
| ATOM | 2298 | NE2 | GLN | A | 314 | 32.862 | 50.247 | 23.920 | 1.00 | 40.87 |
| ATOM | 2299 | N | TRP | A | 315 | 28.880 | 53.258 | 27.298 | 1.00 | 28.07 |
| ATOM | 2300 | CA | TRP | A | 315 | 28.799 | 53.572 | 28.714 | 1.00 | 27.91 |
| ATOM | 2301 | C | TRP | A | 315 | 28.931 | 52.310 | 29.508 | 1.00 | 27.90 |
| ATOM | 2302 | O | TRP | A | 315 | 28.627 | 51.256 | 28.997 | 1.00 | 26.86 |
| ATOM | 2303 | CB | TRP | A | 315 | 27.465 | 54.183 | 29.098 | 1.00 | 27.41 |
| ATOM | 2304 | CG | TRP | A | 315 | 27.037 | 55.330 | 28.340 | 1.00 | 27.24 |
| ATOM | 2305 | CD1 | TRP | A | 315 | 26.389 | 55.330 | 27.128 | 1.00 | 28.62 |
| ATOM | 2306 | CD2 | TRP | A | 315 | 27.125 | 56.681 | 28.737 | 1.00 | 27.34 |
| ATOM | 2307 | NE1 | TRP | A | 315 | 26.091 | 56.609 | 26.753 | 1.00 | 25.10 |
| ATOM | 2308 | CE2 | TRP | A | 315 | 26.530 | 57.457 | 27.729 | 1.00 | 25.13 |
| ATOM | 2309 | CE3 | TRP | A | 315 | 27.656 | 57.332 | 29.853 | 1.00 | 26.34 |
| ATOM | 2310 | CZ2 | TRP | A | 315 | 26.487 | 58.815 | 27.790 | 1.00 | 26.49 |
| ATOM | 2311 | CZ3 | TRP | A | 315 | 27.591 | 58.674 | 29.915 | 1.00 | 24.80 |
| ATOM | 2312 | CH2 | TRP | A | 315 | 27.019 | 59.408 | 28.896 | 1.00 | 26.78 |
| ATOM | 2313 | N | LEU | A | 316 | 29.341 | 52.436 | 30.786 | 1.00 | 27.28 |
| ATOM | 2314 | CA | LEU | A | 316 | 29.622 | 51.274 | 31.623 | 1.00 | 27.37 |
| ATOM | 2315 | C | LEU | A | 316 | 29.134 | 51.583 | 33.017 | 1.00 | 26.51 |
| ATOM | 2316 | O | LEU | A | 316 | 29.402 | 52.667 | 33.511 | 1.00 | 24.17 |
| ATOM | 2317 | CB | LEU | A | 316 | 31.138 | 51.125 | 31.756 | 1.00 | 27.59 |
| ATOM | 2318 | CG | LEU | A | 316 | 31.977 | 49.849 | 31.837 | 1.00 | 29.63 |
| ATOM | 2319 | CD1 | LEU | A | 316 | 33.388 | 50.130 | 32.427 | 1.00 | 27.30 |
| ATOM | 2320 | CD2 | LEU | A | 316 | 31.328 | 48.722 | 32.500 | 1.00 | 30.98 |
| ATOM | 2321 | N | ARG | A | 317 | 28.488 | 50.623 | 33.651 | 1.00 | 26.17 |
| ATOM | 2322 | CA | ARG | A | 317 | 28.050 | 50.814 | 35.007 | 1.00 | 29.17 |
| ATOM | 2323 | C | ARG | A | 317 | 29.274 | 50.717 | 35.931 | 1.00 | 29.53 |
| ATOM | 2324 | O | ARG | A | 317 | 30.220 | 50.025 | 35.624 | 1.00 | 30.93 |
| ATOM | 2325 | CB | ARG | A | 317 | 27.082 | 49.738 | 35.393 | 1.00 | 28.87 |
| ATOM | 2326 | CG | ARG | A | 317 | 25.693 | 49.932 | 34.913 | 1.00 | 31.02 |
| ATOM | 2327 | CD | ARG | A | 317 | 24.699 | 48.952 | 35.588 | 1.00 | 33.50 |
| ATOM | 2328 | NE | ARG | A | 317 | 23.383 | 49.133 | 35.025 | 1.00 | 38.70 |
| ATOM | 2329 | CZ | ARG | A | 317 | 22.459 | 48.190 | 34.950 | 1.00 | 41.63 |
| ATOM | 2330 | NH1 | ARG | A | 317 | 22.688 | 46.961 | 35.416 | 1.00 | 40.89 |
| ATOM | 2331 | NH2 | ARG | A | 317 | 21.299 | 48.482 | 34.390 | 1.00 | 41.28 |
| ATOM | 2332 | N | ARG | A | 318 | 29.267 | 51.436 | 37.028 | 1.00 | 30.66 |
| ATOM | 2333 | CA | ARG | A | 318 | 30.347 | 51.342 | 38.009 | 1.00 | 31.11 |
| ATOM | 2334 | C | ARG | A | 318 | 30.624 | 49.893 | 38.414 | 1.00 | 31.80 |
| ATOM | 2335 | O | ARG | A | 318 | 31.767 | 49.506 | 38.617 | 1.00 | 31.07 |
| ATOM | 2336 | CB | ARG | A | 318 | 30.023 | 52.190 | 39.224 | 1.00 | 31.69 |
| ATOM | 2337 | CG | ARG | A | 318 | 31.204 | 52.357 | 40.138 | 1.00 | 29.53 |
| ATOM | 2338 | CD | ARG | A | 318 | 31.040 | 53.395 | 41.175 | 1.00 | 29.39 |
| ATOM | 2339 | NE | ARG | A | 318 | 32.171 | 53.368 | 42.092 | 1.00 | 29.21 |
| ATOM | 2340 | CZ | ARG | A | 318 | 32.517 | 54.357 | 42.906 | 1.00 | 30.35 |
| ATOM | 2341 | NH1 | ARG | A | 318 | 31.801 | 55.464 | 42.957 | 1.00 | 28.27 |
| ATOM | 2342 | NH2 | ARG | A | 318 | 33.596 | 54.228 | 43.686 | 1.00 | 30.41 |
| ATOM | 2343 | N | ILE | A | 319 | 29.589 | 49.085 | 38.566 | 1.00 | 33.43 |
| ATOM | 2344 | CA | ILE | A | 319 | 29.831 | 47.650 | 38.626 | 1.00 | 34.95 |
| ATOM | 2345 | C | ILE | A | 319 | 30.090 | 47.290 | 37.171 | 1.00 | 35.08 |
| ATOM | 2346 | O | ILE | A | 319 | 29.178 | 47.125 | 36.365 | 1.00 | 34.25 |
| ATOM | 2347 | CB | ILE | A | 319 | 28.662 | 46.854 | 39.188 | 1.00 | 35.85 |
| ATOM | 2348 | CG1 | ILE | A | 319 | 28.281 | 47.352 | 40.575 | 1.00 | 40.14 |
| ATOM | 2349 | CG2 | ILE | A | 319 | 29.108 | 45.402 | 39.371 | 1.00 | 38.30 |
| ATOM | 2350 | CD1 | ILE | A | 319 | 27.121 | 46.470 | 41.213 | 1.00 | 44.56 |
| ATOM | 2351 | N | GLN | A | 320 | 31.357 | 47.171 | 36.829 | 1.00 | 35.11 |
| ATOM | 2352 | CA | GLN | A | 320 | 31.736 | 47.069 | 35.452 | 1.00 | 35.60 |
| ATOM | 2353 | C | GLN | A | 320 | 31.364 | 45.712 | 34.795 | 1.00 | 36.40 |
| ATOM | 2354 | O | GLN | A | 320 | 32.186 | 45.096 | 34.097 | 1.00 | 35.91 |
| ATOM | 2355 | CB | GLN | A | 320 | 33.207 | 47.403 | 35.386 | 1.00 | 35.70 |
| ATOM | 2356 | CG | GLN | A | 320 | 33.477 | 48.794 | 35.908 | 1.00 | 34.45 |
| ATOM | 2357 | CD | GLN | A | 320 | 34.925 | 49.180 | 35.785 | 1.00 | 34.19 |
| ATOM | 2358 | OE1 | GLN | A | 320 | 35.591 | 48.778 | 34.839 | 1.00 | 33.00 |
| ATOM | 2359 | NE2 | GLN | A | 320 | 35.415 | 49.990 | 36.727 | 1.00 | 30.75 |
| ATOM | 2360 | N | ASN | A | 321 | 30.103 | 45.303 | 35.020 | 1.00 | 36.78 |
| ATOM | 2361 | CA | ASN | A | 321 | 29.532 | 44.058 | 34.497 | 1.00 | 37.33 |
| ATOM | 2362 | C | ASN | A | 321 | 28.406 | 44.271 | 33.469 | 1.00 | 36.63 |
| ATOM | 2363 | O | ASN | A | 321 | 27.810 | 43.295 | 33.003 | 1.00 | 36.78 |
| ATOM | 2364 | CB | ASN | A | 321 | 28.996 | 43.127 | 35.629 | 1.00 | 37.05 |
| ATOM | 2365 | CG | ASN | A | 321 | 27.778 | 43.677 | 36.385 | 1.00 | 40.00 |
| ATOM | 2366 | OD1 | ASN | A | 321 | 27.238 | 44.766 | 36.135 | 1.00 | 43.74 |
| ATOM | 2367 | ND2 | ASN | A | 321 | 27.341 | 42.891 | 37.361 | 1.00 | 46.51 |
| ATOM | 2368 | N | TYR | A | 322 | 28.133 | 45.528 | 33.125 | 1.00 | 35.10 |
| ATOM | 2369 | CA | TYR | A | 322 | 27.088 | 45.850 | 32.197 | 1.00 | 34.16 |
| ATOM | 2370 | C | TYR | A | 322 | 27.468 | 47.149 | 31.455 | 1.00 | 33.35 |
| ATOM | 2371 | O | TYR | A | 322 | 27.757 | 48.162 | 32.086 | 1.00 | 32.02 |
| ATOM | 2372 | CB | TYR | A | 322 | 25.785 | 45.988 | 33.007 | 1.00 | 33.91 |
| ATOM | 2373 | CG | TYR | A | 322 | 24.508 | 46.086 | 32.191 | 1.00 | 35.53 |
| ATOM | 2374 | CD1 | TYR | A | 322 | 24.048 | 47.306 | 31.748 | 1.00 | 34.64 |
| ATOM | 2375 | CD2 | TYR | A | 322 | 23.745 | 44.956 | 31.906 | 1.00 | 36.13 |
| ATOM | 2376 | CE1 | TYR | A | 322 | 22.907 | 47.406 | 31.082 | 1.00 | 35.86 |
| ATOM | 2377 | CE2 | TYR | A | 322 | 22.593 | 45.057 | 31.236 | 1.00 | 35.40 |
| ATOM | 2378 | CZ | TYR | A | 322 | 22.179 | 46.282 | 30.804 | 1.00 | 37.49 |
| ATOM | 2379 | OH | TYR | A | 322 | 21.008 | 46.425 | 30.078 | 1.00 | 41.58 |
| ATOM | 2380 | N | SER | A | 323 | 27.517 | 47.104 | 30.126 | 1.00 | 32.85 |
| ATOM | 2381 | CA | SER | A | 323 | 27.810 | 48.283 | 29.315 | 1.00 | 32.99 |
| ATOM | 2382 | C | SER | A | 323 | 26.816 | 48.444 | 28.215 | 1.00 | 32.86 |
| ATOM | 2383 | O | SER | A | 323 | 26.299 | 47.487 | 27.690 | 1.00 | 32.06 |
| ATOM | 2384 | CB | SER | A | 323 | 29.175 | 48.177 | 28.647 | 1.00 | 33.46 |
| ATOM | 2385 | OG | SER | A | 323 | 29.331 | 46.915 | 28.052 | 1.00 | 35.41 |
| ATOM | 2386 | N | VAL | A | 324 | 26.584 | 49.673 | 27.822 | 1.00 | 33.68 |
| ATOM | 2387 | CA | VAL | A | 324 | 25.597 | 49.916 | 26.828 | 1.00 | 34.23 |
| ATOM | 2388 | C | VAL | A | 324 | 26.176 | 50.801 | 25.759 | 1.00 | 34.59 |
| ATOM | 2389 | O | VAL | A | 324 | 26.716 | 51.870 | 26.050 | 1.00 | 34.10 |
| ATOM | 2390 | CB | VAL | A | 324 | 24.380 | 50.610 | 27.434 | 1.00 | 34.28 |
| ATOM | 2391 | CG1 | VAL | A | 324 | 23.463 | 51.091 | 26.331 | 1.00 | 35.67 |
| ATOM | 2392 | CG2 | VAL | A | 324 | 23.605 | 49.672 | 28.402 | 1.00 | 34.51 |
| ATOM | 2393 | N | MET | A | 325 | 26.021 | 50.353 | 24.519 | 1.00 | 35.21 |
| ATOM | 2394 | CA | MET | A | 325 | 26.384 | 51.140 | 23.367 | 1.00 | 36.29 |
| ATOM | 2395 | C | MET | A | 325 | 25.102 | 51.785 | 22.865 | 1.00 | 36.58 |
| ATOM | 2396 | O | MET | A | 325 | 24.146 | 51.079 | 22.504 | 1.00 | 36.39 |
| ATOM | 2397 | CB | MET | A | 325 | 26.995 | 50.268 | 22.262 | 1.00 | 36.35 |
| ATOM | 2398 | CG | MET | A | 325 | 28.170 | 50.923 | 21.505 | 1.00 | 38.37 |
| ATOM | 2399 | SD | MET | A | 325 | 28.923 | 49.807 | 20.259 | 1.00 | 42.06 |
| ATOM | 2400 | CE | MET | A | 325 | 27.992 | 50.257 | 18.898 | 1.00 | 36.19 |
| ATOM | 2401 | N | ASP | A | 326 | 25.095 | 53.119 | 22.875 | 1.00 | 36.53 |
| ATOM | 2402 | CA | ASP | A | 326 | 24.010 | 53.917 | 22.341 | 1.00 | 36.73 |
| ATOM | 2403 | C | ASP | A | 326 | 24.376 | 54.427 | 20.980 | 1.00 | 36.99 |
| ATOM | 2404 | O | ASP | A | 326 | 25.490 | 54.847 | 20.759 | 1.00 | 37.13 |
| ATOM | 2405 | CB | ASP | A | 326 | 23.785 | 55.118 | 23.215 | 1.00 | 37.30 |
| ATOM | 2406 | CG | ASP | A | 326 | 22.371 | 55.262 | 23.618 | 1.00 | 39.85 |
| ATOM | 2407 | OD1 | ASP | A | 326 | 21.773 | 54.229 | 23.962 | 1.00 | 41.00 |
| ATOM | 2408 | OD2 | ASP | A | 326 | 21.762 | 56.357 | 23.611 | 1.00 | 44.95 |
| ATOM | 2409 | N | ILE | A | 327 | 23.433 | 54.411 | 20.052 | 1.00 | 37.51 |
| ATOM | 2410 | CA | ILE | A | 327 | 23.671 | 54.939 | 18.732 | 1.00 | 38.16 |
| ATOM | 2411 | C | ILE | A | 327 | 22.793 | 56.160 | 18.535 | 1.00 | 38.52 |
| ATOM | 2412 | O | ILE | A | 327 | 21.605 | 56.040 | 18.493 | 1.00 | 38.42 |
| ATOM | 2413 | CB | ILE | A | 327 | 23.373 | 53.832 | 17.703 | 1.00 | 38.43 |
| ATOM | 2414 | CG1 | ILE | A | 327 | 24.494 | 52.804 | 17.757 | 1.00 | 39.97 |
| ATOM | 2415 | CG2 | ILE | A | 327 | 23.305 | 54.383 | 16.298 | 1.00 | 37.48 |
| ATOM | 2416 | CD1 | ILE | A | 327 | 24.109 | 51.511 | 17.098 | 1.00 | 43.71 |
| ATOM | 2417 | N | CYS | A | 328 | 23.387 | 57.326 | 18.340 | 1.00 | 39.37 |
| ATOM | 2418 | CA | CYS | A | 328 | 22.631 | 58.571 | 18.312 | 1.00 | 39.97 |
| ATOM | 2419 | C | CYS | A | 328 | 22.714 | 59.309 | 16.993 | 1.00 | 40.41 |
| ATOM | 2420 | O | CYS | A | 328 | 23.804 | 59.495 | 16.454 | 1.00 | 39.69 |
| ATOM | 2421 | CB | CYS | A | 328 | 23.174 | 59.487 | 19.406 | 1.00 | 40.48 |
| ATOM | 2422 | SG | CYS | A | 328 | 23.240 | 58.708 | 21.027 | 1.00 | 42.44 |
| ATOM | 2423 | N | ASP | A | 329 | 21.563 | 59.793 | 16.517 | 1.00 | 40.89 |
| ATOM | 2424 | CA | ASP | A | 329 | 21.473 | 60.435 | 15.213 | 1.00 | 41.39 |
| ATOM | 2425 | C | ASP | A | 329 | 20.974 | 61.877 | 15.270 | 1.00 | 41.92 |
| ATOM | 2426 | O | ASP | A | 329 | 20.119 | 62.198 | 16.058 | 1.00 | 40.55 |
| ATOM | 2427 | CB | ASP | A | 329 | 20.520 | 59.611 | 14.334 | 1.00 | 41.65 |
| ATOM | 2428 | CG | ASP | A | 329 | 21.078 | 58.250 | 13.997 | 1.00 | 42.09 |
| ATOM | 2429 | OD1 | ASP | A | 329 | 22.316 | 58.073 | 13.960 | 1.00 | 42.62 |
| ATOM | 2430 | OD2 | ASP | A | 329 | 20.361 | 57.295 | 13.707 | 1.00 | 45.91 |
| ATOM | 2431 | N | TYR | A | 330 | 21.539 | 62.732 | 14.425 | 1.00 | 43.37 |
| ATOM | 2432 | CA | TYR | A | 330 | 21.146 | 64.118 | 14.330 | 1.00 | 45.55 |
| ATOM | 2433 | C | TYR | A | 330 | 19.844 | 64.297 | 13.567 | 1.00 | 47.23 |
| ATOM | 2434 | O | TYR | A | 330 | 19.696 | 63.790 | 12.479 | 1.00 | 46.53 |
| ATOM | 2435 | CB | TYR | A | 330 | 22.215 | 64.872 | 13.563 | 1.00 | 45.86 |
| ATOM | 2436 | CG | TYR | A | 330 | 21.989 | 66.358 | 13.397 | 1.00 | 46.44 |
| ATOM | 2437 | CD1 | TYR | A | 330 | 21.957 | 67.204 | 14.488 | 1.00 | 47.55 |
| ATOM | 2438 | CD2 | TYR | A | 330 | 21.851 | 66.924 | 12.143 | 1.00 | 48.93 |
| ATOM | 2439 | CE1 | TYR | A | 330 | 21.776 | 68.566 | 14.330 | 1.00 | 47.61 |
| ATOM | 2440 | CE2 | TYR | A | 330 | 21.671 | 68.295 | 11.983 | 1.00 | 47.43 |
| ATOM | 2441 | CZ | TYR | A | 330 | 21.645 | 69.100 | 13.080 | 1.00 | 47.48 |
| ATOM | 2442 | OH | TYR | A | 330 | 21.491 | 70.454 | 12.931 | 1.00 | 49.37 |
| ATOM | 2443 | N | ASP | A | 331 | 18.906 | 65.025 | 14.146 | 1.00 | 50.39 |
| ATOM | 2444 | CA | ASP | A | 331 | 17.643 | 65.331 | 13.482 | 1.00 | 52.45 |
| ATOM | 2445 | C | ASP | A | 331 | 17.730 | 66.709 | 12.850 | 1.00 | 54.21 |
| ATOM | 2446 | O | ASP | A | 331 | 17.660 | 67.719 | 13.541 | 1.00 | 53.88 |
| ATOM | 2447 | CB | ASP | A | 331 | 16.516 | 65.328 | 14.492 | 1.00 | 52.85 |
| ATOM | 2448 | CG | ASP | A | 331 | 15.175 | 65.672 | 13.863 | 1.00 | 53.55 |
| ATOM | 2449 | OD1 | ASP | A | 331 | 15.157 | 66.349 | 12.798 | 1.00 | 55.91 |
| ATOM | 2450 | OD2 | ASP | A | 331 | 14.103 | 65.314 | 14.385 | 1.00 | 51.08 |
| ATOM | 2451 | N | GLU | A | 332 | 17.851 | 66.743 | 11.528 | 1.00 | 56.40 |
| ATOM | 2452 | CA | GLU | A | 332 | 18.076 | 67.986 | 10.804 | 1.00 | 58.14 |
| ATOM | 2453 | C | GLU | A | 332 | 17.075 | 69.081 | 11.142 | 1.00 | 58.88 |
| ATOM | 2454 | O | GLU | A | 332 | 17.434 | 70.247 | 11.216 | 1.00 | 59.23 |
| ATOM | 2455 | CB | GLU | A | 332 | 18.019 | 67.716 | 9.308 | 1.00 | 58.97 |
| ATOM | 2456 | CG | GLU | A | 332 | 18.910 | 68.617 | 8.460 | 1.00 | 61.14 |
| ATOM | 2457 | CD | GLU | A | 332 | 18.759 | 68.313 | 6.972 | 1.00 | 64.90 |
| ATOM | 2458 | OE1 | GLU | A | 332 | 18.225 | 67.222 | 6.643 | 1.00 | 66.07 |
| ATOM | 2459 | OE2 | GLU | A | 332 | 19.156 | 69.164 | 6.132 | 1.00 | 66.87 |
| ATOM | 2460 | N | SER | A | 333 | 15.813 | 68.722 | 11.323 | 1.00 | 59.70 |
| ATOM | 2461 | CA | SER | A | 333 | 14.809 | 69.746 | 11.558 | 1.00 | 60.31 |
| ATOM | 2462 | C | SER | A | 333 | 14.873 | 70.137 | 13.026 | 1.00 | 60.35 |
| ATOM | 2463 | O | SER | A | 333 | 15.106 | 71.291 | 13.356 | 1.00 | 60.53 |
| ATOM | 2464 | CB | SER | A | 333 | 13.410 | 69.256 | 11.160 | 1.00 | 60.41 |
| ATOM | 2465 | OG | SER | A | 333 | 12.753 | 68.607 | 12.238 | 1.00 | 61.18 |
| ATOM | 2466 | N | SER | A | 334 | 14.721 | 69.149 | 13.894 | 1.00 | 60.21 |
| ATOM | 2467 | CA | SER | A | 334 | 14.773 | 69.363 | 15.330 | 1.00 | 60.35 |
| ATOM | 2468 | C | SER | A | 334 | 16.047 | 70.059 | 15.751 | 1.00 | 59.88 |
| ATOM | 2469 | O | SER | A | 334 | 16.044 | 70.869 | 16.662 | 1.00 | 60.48 |
| ATOM | 2470 | CB | SER | A | 334 | 14.699 | 68.013 | 16.035 | 1.00 | 60.48 |
| ATOM | 2471 | OG | SER | A | 334 | 14.620 | 68.147 | 17.435 | 1.00 | 61.75 |
| ATOM | 2472 | N | GLY | A | 335 | 17.141 | 69.740 | 15.074 | 1.00 | 59.42 |
| ATOM | 2473 | CA | GLY | A | 335 | 18.452 | 70.221 | 15.452 | 1.00 | 58.81 |
| ATOM | 2474 | C | GLY | A | 335 | 19.000 | 69.388 | 16.602 | 1.00 | 58.03 |
| ATOM | 2475 | O | GLY | A | 335 | 20.082 | 69.665 | 17.119 | 1.00 | 58.62 |
| ATOM | 2476 | N | ARG | A | 336 | 18.267 | 68.349 | 16.991 | 1.00 | 57.08 |
| ATOM | 2477 | CA | ARG | A | 336 | 18.621 | 67.551 | 18.169 | 1.00 | 56.39 |
| ATOM | 2478 | C | ARG | A | 336 | 19.324 | 66.225 | 17.858 | 1.00 | 54.43 |
| ATOM | 2479 | O | ARG | A | 336 | 19.731 | 65.982 | 16.721 | 1.00 | 53.58 |
| ATOM | 2480 | CB | ARG | A | 336 | 17.360 | 67.309 | 19.006 | 1.00 | 57.25 |
| ATOM | 2481 | CG | ARG | A | 336 | 16.756 | 68.602 | 19.551 | 1.00 | 59.72 |
| ATOM | 2482 | CD | ARG | A | 336 | 15.922 | 68.451 | 20.822 | 1.00 | 63.58 |
| ATOM | 2483 | NE | ARG | A | 336 | 15.666 | 69.766 | 21.422 | 1.00 | 66.15 |
| ATOM | 2484 | CZ | ARG | A | 336 | 16.409 | 70.339 | 22.375 | 1.00 | 68.29 |
| ATOM | 2485 | NH1 | ARG | A | 336 | 17.474 | 69.728 | 22.900 | 1.00 | 66.97 |
| ATOM | 2486 | NH2 | ARG | A | 336 | 16.068 | 71.542 | 22.816 | 1.00 | 69.67 |
| ATOM | 2487 | N | TRP | A | 337 | 19.498 | 65.396 | 18.891 | 1.00 | 52.10 |
| ATOM | 2488 | CA | TRP | A | 337 | 20.141 | 64.088 | 18.749 | 1.00 | 50.39 |
| ATOM | 2489 | C | TRP | A | 337 | 19.307 | 63.065 | 19.457 | 1.00 | 50.29 |
| ATOM | 2490 | O | TRP | A | 337 | 18.958 | 63.240 | 20.617 | 1.00 | 50.80 |
| ATOM | 2491 | CB | TRP | A | 337 | 21.549 | 64.062 | 19.358 | 1.00 | 49.01 |
| ATOM | 2492 | CG | TRP | A | 337 | 22.502 | 64.856 | 18.613 | 1.00 | 43.93 |
| ATOM | 2493 | CD1 | TRP | A | 337 | 22.748 | 66.176 | 18.766 | 1.00 | 41.17 |
| ATOM | 2494 | CD2 | TRP | A | 337 | 23.372 | 64.403 | 17.582 | 1.00 | 38.95 |
| ATOM | 2495 | NE1 | TRP | A | 337 | 23.704 | 66.586 | 17.874 | 1.00 | 38.20 |
| ATOM | 2496 | CE2 | TRP | A | 337 | 24.103 | 65.510 | 17.135 | 1.00 | 37.28 |
| ATOM | 2497 | CE3 | TRP | A | 337 | 23.565 | 63.182 | 16.945 | 1.00 | 36.08 |
| ATOM | 2498 | CZ2 | TRP | A | 337 | 25.012 | 65.429 | 16.112 | 1.00 | 36.93 |
| ATOM | 2499 | CZ3 | TRP | A | 337 | 24.475 | 63.106 | 15.948 | 1.00 | 35.12 |
| ATOM | 2500 | CH2 | TRP | A | 337 | 25.192 | 64.215 | 15.539 | 1.00 | 35.09 |
| ATOM | 2501 | N | ASN | A | 338 | 19.013 | 61.974 | 18.777 | 1.00 | 50.08 |
| ATOM | 2502 | CA | ASN | A | 338 | 18.151 | 60.966 | 19.360 | 1.00 | 50.17 |
| ATOM | 2503 | C | ASN | A | 338 | 18.782 | 59.601 | 19.364 | 1.00 | 49.12 |
| ATOM | 2504 | O | ASN | A | 338 | 19.398 | 59.184 | 18.387 | 1.00 | 49.10 |
| ATOM | 2505 | CB | ASN | A | 338 | 16.798 | 60.955 | 18.644 | 1.00 | 50.84 |
| ATOM | 2506 | CG | ASN | A | 338 | 15.967 | 62.160 | 19.000 | 1.00 | 52.32 |
| ATOM | 2507 | OD1 | ASN | A | 338 | 15.284 | 62.170 | 20.028 | 1.00 | 56.68 |
| ATOM | 2508 | ND2 | ASN | A | 338 | 16.045 | 63.201 | 18.179 | 1.00 | 53.70 |
| ATOM | 2509 | N | CYS | A | 339 | 18.623 | 58.912 | 20.476 | 1.00 | 48.04 |
| ATOM | 2510 | CA | CYS | A | 339 | 19.275 | 57.646 | 20.656 | 1.00 | 48.30 |
| ATOM | 2511 | C | CYS | A | 339 | 18.170 | 56.619 | 20.904 | 1.00 | 49.07 |
| ATOM | 2512 | O | CYS | A | 339 | 17.597 | 56.548 | 21.980 | 1.00 | 48.71 |
| ATOM | 2513 | CB | CYS | A | 339 | 20.288 | 57.722 | 21.818 | 1.00 | 47.82 |
| ATOM | 2514 | SG | CYS | A | 339 | 21.401 | 59.178 | 21.776 | 1.00 | 45.14 |
| ATOM | 2515 | N | LEU | A | 340 | 17.901 | 55.818 | 19.887 | 1.00 | 49.54 |
| ATOM | 2516 | CA | LEU | A | 340 | 16.809 | 54.885 | 19.929 | 1.00 | 50.67 |
| ATOM | 2517 | C | LEU | A | 340 | 17.090 | 53.745 | 20.865 | 1.00 | 50.43 |
| ATOM | 2518 | O | LEU | A | 340 | 17.987 | 52.965 | 20.633 | 1.00 | 50.01 |
| ATOM | 2519 | CB | LEU | A | 340 | 16.528 | 54.366 | 18.521 | 1.00 | 50.91 |
| ATOM | 2520 | CG | LEU | A | 340 | 15.056 | 54.371 | 18.159 | 1.00 | 52.84 |
| ATOM | 2521 | CD1 | LEU | A | 340 | 14.381 | 55.656 | 18.678 | 1.00 | 53.31 |
| ATOM | 2522 | CD2 | LEU | A | 340 | 14.893 | 54.230 | 16.644 | 1.00 | 52.52 |
| ATOM | 2523 | N | VAL | A | 341 | 16.275 | 53.620 | 21.904 | 1.00 | 51.21 |
| ATOM | 2524 | CA | VAL | A | 341 | 16.533 | 52.631 | 22.944 | 1.00 | 51.79 |
| ATOM | 2525 | C | VAL | A | 341 | 16.639 | 51.283 | 22.296 | 1.00 | 52.02 |
| ATOM | 2526 | O | VAL | A | 341 | 17.201 | 50.365 | 22.881 | 1.00 | 51.85 |
| ATOM | 2527 | CB | VAL | A | 341 | 15.446 | 52.638 | 24.097 | 1.00 | 52.37 |
| ATOM | 2528 | CG1 | VAL | A | 341 | 16.020 | 52.121 | 25.412 | 1.00 | 52.37 |
| ATOM | 2529 | CG2 | VAL | A | 341 | 14.913 | 54.043 | 24.351 | 1.00 | 53.02 |
| ATOM | 2530 | N | ALA | A | 342 | 16.155 | 51.182 | 21.055 | 1.00 | 52.45 |
| ATOM | 2531 | CA | ALA | A | 342 | 16.142 | 49.910 | 20.326 | 1.00 | 52.38 |
| ATOM | 2532 | C | ALA | A | 342 | 17.364 | 49.561 | 19.496 | 1.00 | 52.19 |
| ATOM | 2533 | O | ALA | A | 342 | 17.554 | 48.368 | 19.164 | 1.00 | 53.38 |
| ATOM | 2534 | CB | ALA | A | 342 | 14.890 | 49.817 | 19.452 | 1.00 | 53.05 |
| ATOM | 2535 | N | ARG | A | 343 | 18.184 | 50.517 | 19.055 | 1.00 | 50.99 |
| ATOM | 2536 | CA | ARG | A | 343 | 19.453 | 50.013 | 18.508 | 1.00 | 49.82 |
| ATOM | 2537 | C | ARG | A | 343 | 20.516 | 49.891 | 19.622 | 1.00 | 47.74 |
| ATOM | 2538 | O | ARG | A | 343 | 21.673 | 49.616 | 19.353 | 1.00 | 47.28 |
| ATOM | 2539 | CB | ARG | A | 343 | 19.980 | 50.735 | 17.255 | 1.00 | 49.82 |
| ATOM | 2540 | CG | ARG | A | 343 | 20.200 | 52.183 | 17.356 | 1.00 | 50.27 |
| ATOM | 2541 | CD | ARG | A | 343 | 19.170 | 52.981 | 16.676 | 1.00 | 51.54 |
| ATOM | 2542 | NE | ARG | A | 343 | 19.158 | 52.772 | 15.245 | 1.00 | 51.73 |
| ATOM | 2543 | CZ | ARG | A | 343 | 18.601 | 53.611 | 14.391 | 1.00 | 51.85 |
| ATOM | 2544 | NH1 | ARG | A | 343 | 18.023 | 54.705 | 14.840 | 1.00 | 48.59 |
| ATOM | 2545 | NH2 | ARG | A | 343 | 18.637 | 53.363 | 13.086 | 1.00 | 54.27 |
| ATOM | 2546 | N | GLN | A | 344 | 20.114 | 50.055 | 20.875 | 1.00 | 46.47 |
| ATOM | 2547 | CA | GLN | A | 344 | 21.075 | 49.932 | 21.967 | 1.00 | 44.89 |
| ATOM | 2548 | C | GLN | A | 344 | 21.723 | 48.582 | 21.927 | 1.00 | 44.26 |
| ATOM | 2549 | O | GLN | A | 344 | 21.055 | 47.590 | 21.800 | 1.00 | 43.75 |
| ATOM | 2550 | CB | GLN | A | 344 | 20.408 | 50.082 | 23.318 | 1.00 | 44.84 |
| ATOM | 2551 | CG | GLN | A | 344 | 20.271 | 51.483 | 23.815 | 1.00 | 43.22 |
| ATOM | 2552 | CD | GLN | A | 344 | 19.672 | 51.530 | 25.190 | 1.00 | 44.80 |
| ATOM | 2553 | OE1 | GLN | A | 344 | 19.569 | 50.492 | 25.868 | 1.00 | 42.35 |
| ATOM | 2554 | NE2 | GLN | A | 344 | 19.263 | 52.732 | 25.619 | 1.00 | 43.12 |
| ATOM | 2555 | N | HIS | A | 345 | 23.036 | 48.526 | 21.989 | 1.00 | 43.91 |
| ATOM | 2556 | CA | HIS | A | 345 | 23.655 | 47.223 | 22.119 | 1.00 | 44.32 |
| ATOM | 2557 | C | HIS | A | 345 | 24.208 | 47.031 | 23.533 | 1.00 | 43.74 |
| ATOM | 2558 | O | HIS | A | 345 | 24.812 | 47.927 | 24.104 | 1.00 | 43.43 |
| ATOM | 2559 | CB | HIS | A | 345 | 24.701 | 47.022 | 21.049 | 1.00 | 44.71 |
| ATOM | 2560 | CG | HIS | A | 345 | 24.118 | 46.552 | 19.758 | 1.00 | 46.48 |
| ATOM | 2561 | ND1 | HIS | A | 345 | 23.849 | 47.406 | 18.709 | 1.00 | 50.06 |
| ATOM | 2562 | CD2 | HIS | A | 345 | 23.717 | 45.321 | 19.359 | 1.00 | 47.51 |
| ATOM | 2563 | CE1 | HIS | A | 345 | 23.335 | 46.714 | 17.704 | 1.00 | 50.97 |
| ATOM | 2564 | NE2 | HIS | A | 345 | 23.250 | 45.445 | 18.071 | 1.00 | 49.88 |
| ATOM | 2565 | N | ILE | A | 346 | 24.010 | 45.839 | 24.068 | 1.00 | 44.11 |
| ATOM | 2566 | CA | ILE | A | 346 | 24.321 | 45.533 | 25.456 | 1.00 | 44.80 |
| ATOM | 2567 | C | ILE | A | 346 | 25.406 | 44.498 | 25.572 | 1.00 | 44.66 |
| ATOM | 2568 | O | ILE | A | 346 | 25.445 | 43.579 | 24.782 | 1.00 | 44.38 |
| ATOM | 2569 | CB | ILE | A | 346 | 23.061 | 44.957 | 26.130 | 1.00 | 45.03 |
| ATOM | 2570 | CG1 | ILE | A | 346 | 21.984 | 46.030 | 26.251 | 1.00 | 46.42 |
| ATOM | 2571 | CG2 | ILE | A | 346 | 23.403 | 44.349 | 27.485 | 1.00 | 45.11 |
| ATOM | 2572 | CD1 | ILE | A | 346 | 20.698 | 45.504 | 26.887 | 1.00 | 48.78 |
| ATOM | 2573 | N | GLU | A | 347 | 26.281 | 44.647 | 26.561 | 1.00 | 44.63 |
| ATOM | 2574 | CA | GLU | A | 347 | 27.304 | 43.634 | 26.846 | 1.00 | 45.49 |
| ATOM | 2575 | C | GLU | A | 347 | 27.319 | 43.349 | 28.332 | 1.00 | 46.21 |
| ATOM | 2576 | O | GLU | A | 347 | 27.265 | 44.287 | 29.130 | 1.00 | 46.36 |
| ATOM | 2577 | CB | GLU | A | 347 | 28.683 | 44.102 | 26.431 | 1.00 | 45.45 |
| ATOM | 2578 | CG | GLU | A | 347 | 28.840 | 44.218 | 24.941 | 1.00 | 45.59 |
| ATOM | 2579 | CD | GLU | A | 347 | 30.230 | 44.657 | 24.535 | 1.00 | 46.13 |
| ATOM | 2580 | OE1 | GLU | A | 347 | 31.228 | 44.114 | 25.070 | 1.00 | 44.42 |
| ATOM | 2581 | OE2 | GLU | A | 347 | 30.305 | 45.522 | 23.641 | 1.00 | 48.13 |
| ATOM | 2582 | N | MET | A | 348 | 27.383 | 42.064 | 28.692 | 1.00 | 46.97 |
| ATOM | 2583 | CA | MET | A | 348 | 27.417 | 41.624 | 30.080 | 1.00 | 47.81 |
| ATOM | 2584 | C | MET | A | 348 | 28.436 | 40.512 | 30.213 | 1.00 | 48.11 |
| ATOM | 2585 | O | MET | A | 348 | 28.596 | 39.690 | 29.314 | 1.00 | 48.00 |
| ATOM | 2586 | CB | MET | A | 348 | 26.072 | 41.037 | 30.495 | 1.00 | 48.89 |
| ATOM | 2587 | CG | MET | A | 348 | 24.850 | 41.803 | 30.045 | 1.00 | 52.13 |
| ATOM | 2588 | SD | MET | A | 348 | 23.299 | 41.001 | 30.546 | 1.00 | 57.88 |
| ATOM | 2589 | CE | MET | A | 348 | 23.747 | 39.227 | 30.232 | 1.00 | 58.62 |
| ATOM | 2590 | N | SER | A | 349 | 29.153 | 40.449 | 31.319 | 1.00 | 48.36 |
| ATOM | 2591 | CA | SER | A | 349 | 30.059 | 39.325 | 31.465 | 1.00 | 48.44 |
| ATOM | 2592 | C | SER | A | 349 | 29.454 | 38.520 | 32.551 | 1.00 | 48.12 |
| ATOM | 2593 | O | SER | A | 349 | 28.822 | 39.068 | 33.453 | 1.00 | 48.86 |
| ATOM | 2594 | CB | SER | A | 349 | 31.497 | 39.739 | 31.816 | 1.00 | 49.01 |
| ATOM | 2595 | OG | SER | A | 349 | 32.299 | 38.621 | 32.216 | 1.00 | 47.97 |
| ATOM | 2596 | N | THR | A | 350 | 29.617 | 37.214 | 32.456 | 1.00 | 47.84 |
| ATOM | 2597 | CA | THR | A | 350 | 29.142 | 36.342 | 33.507 | 1.00 | 47.83 |
| ATOM | 2598 | C | THR | A | 350 | 30.323 | 36.049 | 34.417 | 1.00 | 46.74 |
| ATOM | 2599 | O | THR | A | 350 | 30.217 | 36.207 | 35.620 | 1.00 | 46.92 |
| ATOM | 2600 | CB | THR | A | 350 | 28.503 | 35.029 | 32.918 | 1.00 | 48.52 |
| ATOM | 2601 | OG1 | THR | A | 350 | 27.077 | 35.036 | 33.128 | 1.00 | 48.99 |
| ATOM | 2602 | CG2 | THR | A | 350 | 28.935 | 33.779 | 33.680 | 1.00 | 49.06 |
| ATOM | 2603 | N | THR | A | 351 | 31.460 | 35.668 | 33.836 | 1.00 | 44.97 |
| ATOM | 2604 | CA | THR | A | 351 | 32.587 | 35.220 | 34.648 | 1.00 | 43.60 |
| ATOM | 2605 | C | THR | A | 351 | 33.506 | 36.322 | 35.211 | 1.00 | 42.20 |
| ATOM | 2606 | O | THR | A | 351 | 34.397 | 36.049 | 36.010 | 1.00 | 42.09 |
| ATOM | 2607 | CB | THR | A | 351 | 33.377 | 34.161 | 33.872 | 1.00 | 44.01 |
| ATOM | 2608 | OG1 | THR | A | 351 | 33.740 | 34.647 | 32.574 | 1.00 | 43.68 |
| ATOM | 2609 | CG2 | THR | A | 351 | 32.481 | 32.935 | 33.563 | 1.00 | 43.10 |
| ATOM | 2610 | N | GLY | A | 352 | 33.256 | 37.575 | 34.838 | 1.00 | 40.20 |
| ATOM | 2611 | CA | GLY | A | 352 | 34.104 | 38.652 | 35.275 | 1.00 | 37.56 |
| ATOM | 2612 | C | GLY | A | 352 | 33.645 | 40.018 | 34.828 | 1.00 | 35.69 |
| ATOM | 2613 | O | GLY | A | 352 | 32.492 | 40.400 | 35.022 | 1.00 | 33.74 |
| ATOM | 2614 | N | TRP | A | 353 | 34.549 | 40.757 | 34.199 | 1.00 | 32.99 |
| ATOM | 2615 | CA | TRP | A | 353 | 34.260 | 42.148 | 33.875 | 1.00 | 31.53 |
| ATOM | 2616 | C | TRP | A | 353 | 34.108 | 42.268 | 32.365 | 1.00 | 30.70 |
| ATOM | 2617 | O | TRP | A | 353 | 34.170 | 41.276 | 31.690 | 1.00 | 31.36 |
| ATOM | 2618 | CB | TRP | A | 353 | 35.384 | 43.033 | 34.441 | 1.00 | 30.98 |
| ATOM | 2619 | CG | TRP | A | 353 | 36.767 | 42.617 | 33.951 | 1.00 | 26.61 |
| ATOM | 2620 | CD1 | TRP | A | 353 | 37.385 | 43.116 | 32.901 | 1.00 | 22.83 |
| ATOM | 2621 | CD2 | TRP | A | 353 | 37.668 | 41.653 | 34.533 | 1.00 | 21.90 |
| ATOM | 2622 | NE1 | TRP | A | 353 | 38.637 | 42.556 | 32.764 | 1.00 | 26.63 |
| ATOM | 2623 | CE2 | TRP | A | 353 | 38.816 | 41.631 | 33.743 | 1.00 | 24.09 |
| ATOM | 2624 | CE3 | TRP | A | 353 | 37.623 | 40.821 | 35.648 | 1.00 | 21.95 |
| ATOM | 2625 | CZ2 | TRP | A | 353 | 39.899 | 40.814 | 34.006 | 1.00 | 23.28 |
| ATOM | 2626 | CZ3 | TRP | A | 353 | 38.715 | 39.999 | 35.926 | 1.00 | 18.74 |
| ATOM | 2627 | CH2 | TRP | A | 353 | 39.828 | 40.007 | 35.099 | 1.00 | 23.86 |
| ATOM | 2628 | N | VAL | A | 354 | 33.911 | 43.460 | 31.833 | 1.00 | 30.38 |
| ATOM | 2629 | CA | VAL | A | 354 | 33.693 | 43.639 | 30.388 | 1.00 | 30.47 |
| ATOM | 2630 | C | VAL | A | 354 | 34.944 | 44.147 | 29.691 | 1.00 | 30.78 |
| ATOM | 2631 | O | VAL | A | 354 | 35.436 | 45.236 | 29.995 | 1.00 | 31.50 |
| ATOM | 2632 | CB | VAL | A | 354 | 32.598 | 44.726 | 30.118 | 1.00 | 29.56 |
| ATOM | 2633 | CG1 | VAL | A | 354 | 32.331 | 44.886 | 28.720 | 1.00 | 29.31 |
| ATOM | 2634 | CG2 | VAL | A | 354 | 31.310 | 44.383 | 30.775 | 1.00 | 29.98 |
| ATOM | 2635 | N | GLY | A | 355 | 35.437 | 43.373 | 28.745 | 1.00 | 30.87 |
| ATOM | 2636 | CA | GLY | A | 355 | 36.612 | 43.739 | 27.966 | 1.00 | 31.04 |
| ATOM | 2637 | C | GLY | A | 355 | 37.883 | 43.431 | 28.714 | 1.00 | 31.16 |
| ATOM | 2638 | O | GLY | A | 355 | 37.868 | 42.748 | 29.745 | 1.00 | 31.10 |
| ATOM | 2639 | N | ARG | A | 356 | 39.001 | 43.944 | 28.215 | 1.00 | 32.24 |
| ATOM | 2640 | CA | ARG | A | 356 | 40.290 | 43.653 | 28.829 | 1.00 | 33.32 |
| ATOM | 2641 | C | ARG | A | 356 | 40.551 | 44.699 | 29.939 | 1.00 | 33.80 |
| ATOM | 2642 | O | ARG | A | 356 | 40.449 | 44.391 | 31.136 | 1.00 | 33.03 |
| ATOM | 2643 | CB | ARG | A | 356 | 41.389 | 43.602 | 27.776 | 1.00 | 33.33 |
| ATOM | 2644 | CG | ARG | A | 356 | 41.468 | 42.265 | 27.012 | 1.00 | 35.53 |
| ATOM | 2645 | CD | ARG | A | 356 | 42.500 | 42.252 | 25.810 | 1.00 | 36.37 |
| ATOM | 2646 | NE | ARG | A | 356 | 42.593 | 40.937 | 25.161 | 1.00 | 39.23 |
| ATOM | 2647 | CZ | ARG | A | 356 | 42.422 | 40.677 | 23.835 | 1.00 | 37.64 |
| ATOM | 2648 | NH1 | ARG | A | 356 | 42.114 | 41.615 | 22.958 | 1.00 | 36.00 |
| ATOM | 2649 | NH2 | ARG | A | 356 | 42.559 | 39.452 | 23.394 | 1.00 | 37.65 |
| ATOM | 2650 | N | PHE | A | 357 | 40.815 | 45.932 | 29.532 | 1.00 | 34.12 |
| ATOM | 2651 | CA | PHE | A | 357 | 40.960 | 47.044 | 30.471 | 1.00 | 35.49 |
| ATOM | 2652 | C | PHE | A | 357 | 39.778 | 47.998 | 30.390 | 1.00 | 35.77 |
| ATOM | 2653 | O | PHE | A | 357 | 39.651 | 48.933 | 31.179 | 1.00 | 34.97 |
| ATOM | 2654 | CB | PHE | A | 357 | 42.269 | 47.761 | 30.192 | 1.00 | 35.85 |
| ATOM | 2655 | CG | PHE | A | 357 | 43.459 | 46.902 | 30.485 | 1.00 | 38.15 |
| ATOM | 2656 | CD1 | PHE | A | 357 | 43.800 | 46.605 | 31.799 | 1.00 | 38.33 |
| ATOM | 2657 | CD2 | PHE | A | 357 | 44.184 | 46.330 | 29.460 | 1.00 | 40.95 |
| ATOM | 2658 | CE1 | PHE | A | 357 | 44.885 | 45.800 | 32.082 | 1.00 | 38.68 |
| ATOM | 2659 | CE2 | PHE | A | 357 | 45.269 | 45.527 | 29.739 | 1.00 | 42.58 |
| ATOM | 2660 | CZ | PHE | A | 357 | 45.618 | 45.261 | 31.057 | 1.00 | 41.21 |
| ATOM | 2661 | N | ARG | A | 358 | 38.897 | 47.706 | 29.437 | 1.00 | 36.06 |
| ATOM | 2662 | CA | ARG | A | 358 | 37.711 | 48.497 | 29.157 | 1.00 | 36.46 |
| ATOM | 2663 | C | ARG | A | 358 | 36.922 | 47.799 | 28.024 | 1.00 | 35.36 |
| ATOM | 2664 | O | ARG | A | 358 | 37.391 | 46.871 | 27.430 | 1.00 | 34.09 |
| ATOM | 2665 | CB | ARG | A | 358 | 38.115 | 49.913 | 28.726 | 1.00 | 36.17 |
| ATOM | 2666 | CG | ARG | A | 358 | 39.145 | 49.958 | 27.558 | 1.00 | 39.77 |
| ATOM | 2667 | CD | ARG | A | 358 | 39.180 | 51.309 | 26.811 | 1.00 | 43.70 |
| ATOM | 2668 | NE | ARG | A | 358 | 40.420 | 52.050 | 26.994 | 1.00 | 48.95 |
| ATOM | 2669 | CZ | ARG | A | 358 | 40.844 | 52.594 | 28.135 | 1.00 | 52.79 |
| ATOM | 2670 | NH1 | ARG | A | 358 | 40.115 | 52.528 | 29.240 | 1.00 | 56.15 |
| ATOM | 2671 | NH2 | ARG | A | 358 | 42.001 | 53.246 | 28.170 | 1.00 | 52.75 |
| ATOM | 2672 | N | PRO | A | 359 | 35.697 | 48.233 | 27.786 | 1.00 | 34.80 |
| ATOM | 2673 | CA | PRO | A | 359 | 34.923 | 47.749 | 26.658 | 1.00 | 33.84 |
| ATOM | 2674 | C | PRO | A | 359 | 35.692 | 47.972 | 25.354 | 1.00 | 33.44 |
| ATOM | 2675 | O | PRO | A | 359 | 36.257 | 49.023 | 25.165 | 1.00 | 32.66 |
| ATOM | 2676 | CB | PRO | A | 359 | 33.701 | 48.654 | 26.688 | 1.00 | 34.51 |
| ATOM | 2677 | CG | PRO | A | 359 | 33.538 | 48.976 | 28.143 | 1.00 | 35.09 |
| ATOM | 2678 | CD | PRO | A | 359 | 34.950 | 49.197 | 28.616 | 1.00 | 34.39 |
| ATOM | 2679 | N | SER | A | 360 | 35.667 | 46.992 | 24.461 | 1.00 | 31.83 |
| ATOM | 2680 | CA | SER | A | 360 | 36.344 | 47.084 | 23.212 | 1.00 | 30.20 |
| ATOM | 2681 | C | SER | A | 360 | 35.705 | 48.140 | 22.314 | 1.00 | 29.79 |
| ATOM | 2682 | O | SER | A | 360 | 34.533 | 48.475 | 22.423 | 1.00 | 29.04 |
| ATOM | 2683 | CB | SER | A | 360 | 36.335 | 45.724 | 22.527 | 1.00 | 30.82 |
| ATOM | 2684 | OG | SER | A | 360 | 35.019 | 45.301 | 22.235 | 1.00 | 29.59 |
| ATOM | 2685 | N | GLU | A | 361 | 36.525 | 48.701 | 21.450 | 1.00 | 29.11 |
| ATOM | 2686 | CA | GLU | A | 361 | 36.077 | 49.715 | 20.532 | 1.00 | 30.22 |
| ATOM | 2687 | C | GLU | A | 361 | 35.319 | 49.111 | 19.332 | 1.00 | 29.39 |
| ATOM | 2688 | O | GLU | A | 361 | 35.743 | 48.116 | 18.782 | 1.00 | 27.57 |
| ATOM | 2689 | CB | GLU | A | 361 | 37.312 | 50.426 | 20.028 | 1.00 | 30.69 |
| ATOM | 2690 | CG | GLU | A | 361 | 37.141 | 51.253 | 18.799 | 1.00 | 34.45 |
| ATOM | 2691 | CD | GLU | A | 361 | 38.464 | 51.764 | 18.297 | 1.00 | 38.00 |
| ATOM | 2692 | OE1 | GLU | A | 361 | 39.487 | 51.227 | 18.751 | 1.00 | 44.33 |
| ATOM | 2693 | OE2 | GLU | A | 361 | 38.490 | 52.699 | 17.475 | 1.00 | 40.62 |
| ATOM | 2694 | N | PRO | A | 362 | 34.228 | 49.754 | 18.941 | 1.00 | 30.05 |
| ATOM | 2695 | CA | PRO | A | 362 | 33.482 | 49.427 | 17.716 | 1.00 | 30.28 |
| ATOM | 2696 | C | PRO | A | 362 | 34.118 | 50.047 | 16.461 | 1.00 | 30.65 |
| ATOM | 2697 | O | PRO | A | 362 | 34.522 | 51.189 | 16.533 | 1.00 | 30.95 |
| ATOM | 2698 | CB | PRO | A | 362 | 32.160 | 50.116 | 17.938 | 1.00 | 30.19 |
| ATOM | 2699 | CG | PRO | A | 362 | 32.417 | 51.216 | 18.892 | 1.00 | 29.85 |
| ATOM | 2700 | CD | PRO | A | 362 | 33.630 | 50.892 | 19.653 | 1.00 | 30.30 |
| ATOM | 2701 | N | HIS | A | 363 | 34.185 | 49.309 | 15.348 | 1.00 | 30.69 |
| ATOM | 2702 | CA | HIS | A | 363 | 34.766 | 49.766 | 14.099 | 1.00 | 31.18 |
| ATOM | 2703 | C | HIS | A | 363 | 33.636 | 49.717 | 13.044 | 1.00 | 32.07 |
| ATOM | 2704 | O | HIS | A | 363 | 33.249 | 48.637 | 12.585 | 1.00 | 32.01 |
| ATOM | 2705 | CB | HIS | A | 363 | 35.898 | 48.828 | 13.709 | 1.00 | 31.43 |
| ATOM | 2706 | CG | HIS | A | 363 | 37.104 | 48.970 | 14.572 | 1.00 | 30.92 |
| ATOM | 2707 | ND1 | HIS | A | 363 | 37.120 | 48.604 | 15.905 | 1.00 | 33.58 |
| ATOM | 2708 | CD2 | HIS | A | 363 | 38.313 | 49.509 | 14.316 | 1.00 | 31.13 |
| ATOM | 2709 | CE1 | HIS | A | 363 | 38.309 | 48.864 | 16.413 | 1.00 | 32.05 |
| ATOM | 2710 | NE2 | HIS | A | 363 | 39.052 | 49.412 | 15.467 | 1.00 | 33.31 |
| ATOM | 2711 | N | PHE | A | 364 | 33.116 | 50.892 | 12.705 | 1.00 | 31.92 |
| ATOM | 2712 | CA | PHE | A | 364 | 31.934 | 51.036 | 11.883 | 1.00 | 32.06 |
| ATOM | 2713 | C | PHE | A | 364 | 32.243 | 50.969 | 10.422 | 1.00 | 32.54 |
| ATOM | 2714 | O | PHE | A | 364 | 33.218 | 51.536 | 9.961 | 1.00 | 31.64 |
| ATOM | 2715 | CB | PHE | A | 364 | 31.233 | 52.387 | 12.149 | 1.00 | 31.72 |
| ATOM | 2716 | CG | PHE | A | 364 | 30.437 | 52.437 | 13.439 | 1.00 | 31.78 |
| ATOM | 2717 | CD1 | PHE | A | 364 | 29.090 | 52.119 | 13.464 | 1.00 | 27.81 |
| ATOM | 2718 | CD2 | PHE | A | 364 | 31.053 | 52.790 | 14.639 | 1.00 | 33.24 |
| ATOM | 2719 | CE1 | PHE | A | 364 | 28.378 | 52.176 | 14.613 | 1.00 | 29.56 |
| ATOM | 2720 | CE2 | PHE | A | 364 | 30.352 | 52.821 | 15.795 | 1.00 | 31.13 |
| ATOM | 2721 | CZ | PHE | A | 364 | 28.986 | 52.518 | 15.784 | 1.00 | 32.61 |
| ATOM | 2722 | N | THR | A | 365 | 31.368 | 50.293 | 9.674 | 1.00 | 33.31 |
| ATOM | 2723 | CA | THR | A | 365 | 31.498 | 50.288 | 8.253 | 1.00 | 34.57 |
| ATOM | 2724 | C | THR | A | 365 | 31.228 | 51.716 | 7.819 | 1.00 | 34.99 |
| ATOM | 2725 | O | THR | A | 365 | 30.651 | 52.496 | 8.546 | 1.00 | 34.77 |
| ATOM | 2726 | CB | THR | A | 365 | 30.504 | 49.317 | 7.601 | 1.00 | 35.03 |
| ATOM | 2727 | OG1 | THR | A | 365 | 29.176 | 49.649 | 8.001 | 1.00 | 38.45 |
| ATOM | 2728 | CG2 | THR | A | 365 | 30.681 | 47.938 | 8.127 | 1.00 | 34.53 |
| ATOM | 2729 | N | LEU | A | 366 | 31.672 | 52.053 | 6.623 | 1.00 | 36.44 |
| ATOM | 2730 | CA | LEU | A | 366 | 31.561 | 53.401 | 6.106 | 1.00 | 37.06 |
| ATOM | 2731 | C | LEU | A | 366 | 30.167 | 53.996 | 6.119 | 1.00 | 37.17 |
| ATOM | 2732 | O | LEU | A | 366 | 30.032 | 55.214 | 6.280 | 1.00 | 37.82 |
| ATOM | 2733 | CB | LEU | A | 366 | 32.056 | 53.415 | 4.667 | 1.00 | 37.55 |
| ATOM | 2734 | CG | LEU | A | 366 | 33.483 | 53.889 | 4.390 | 1.00 | 39.89 |
| ATOM | 2735 | CD1 | LEU | A | 366 | 33.638 | 54.070 | 2.878 | 1.00 | 41.97 |
| ATOM | 2736 | CD2 | LEU | A | 366 | 33.776 | 55.209 | 5.134 | 1.00 | 42.04 |
| ATOM | 2737 | N | ASP | A | 367 | 29.139 | 53.174 | 5.912 | 1.00 | 36.03 |
| ATOM | 2738 | CA | ASP | A | 367 | 27.773 | 53.701 | 5.888 | 1.00 | 36.01 |
| ATOM | 2739 | C | ASP | A | 367 | 27.199 | 53.910 | 7.269 | 1.00 | 34.74 |
| ATOM | 2740 | O | ASP | A | 367 | 26.175 | 54.561 | 7.426 | 1.00 | 34.21 |
| ATOM | 2741 | CB | ASP | A | 367 | 26.812 | 52.825 | 5.054 | 1.00 | 36.66 |
| ATOM | 2742 | CG | ASP | A | 367 | 26.868 | 51.345 | 5.410 | 1.00 | 38.11 |
| ATOM | 2743 | OD1 | ASP | A | 367 | 27.230 | 50.976 | 6.536 | 1.00 | 38.62 |
| ATOM | 2744 | OD2 | ASP | A | 367 | 26.578 | 50.456 | 4.579 | 1.00 | 43.47 |
| ATOM | 2745 | N | GLY | A | 368 | 27.856 | 53.316 | 8.258 | 1.00 | 33.46 |
| ATOM | 2746 | CA | GLY | A | 368 | 27.506 | 53.488 | 9.650 | 1.00 | 32.51 |
| ATOM | 2747 | C | GLY | A | 368 | 26.426 | 52.599 | 10.153 | 1.00 | 31.58 |
| ATOM | 2748 | O | GLY | A | 368 | 25.992 | 52.784 | 11.281 | 1.00 | 30.96 |
| ATOM | 2749 | N | ASN | A | 369 | 25.998 | 51.633 | 9.334 | 1.00 | 30.28 |
| ATOM | 2750 | CA | ASN | A | 369 | 24.828 | 50.848 | 9.660 | 1.00 | 29.27 |
| ATOM | 2751 | C | ASN | A | 369 | 25.249 | 49.571 | 10.281 | 1.00 | 28.67 |
| ATOM | 2752 | O | ASN | A | 369 | 24.417 | 48.759 | 10.684 | 1.00 | 28.60 |
| ATOM | 2753 | CB | ASN | A | 369 | 24.027 | 50.569 | 8.372 | 1.00 | 30.29 |
| ATOM | 2754 | CG | ASN | A | 369 | 23.406 | 51.857 | 7.752 | 1.00 | 29.97 |
| ATOM | 2755 | OD1 | ASN | A | 369 | 23.046 | 52.775 | 8.459 | 1.00 | 30.57 |
| ATOM | 2756 | ND2 | ASN | A | 369 | 23.263 | 51.880 | 6.433 | 1.00 | 31.70 |
| ATOM | 2757 | N | SER | A | 370 | 26.561 | 49.372 | 10.370 | 1.00 | 27.14 |
| ATOM | 2758 | CA | SER | A | 370 | 27.082 | 48.168 | 10.954 | 1.00 | 26.22 |
| ATOM | 2759 | C | SER | A | 370 | 28.511 | 48.399 | 11.463 | 1.00 | 25.28 |
| ATOM | 2760 | O | SER | A | 370 | 29.195 | 49.343 | 11.038 | 1.00 | 21.89 |
| ATOM | 2761 | CB | SER | A | 370 | 27.082 | 47.027 | 9.927 | 1.00 | 25.88 |
| ATOM | 2762 | OG | SER | A | 370 | 27.952 | 47.340 | 8.858 | 1.00 | 30.29 |
| ATOM | 2763 | N | PHE | A | 371 | 28.929 | 47.505 | 12.362 | 1.00 | 25.10 |
| ATOM | 2764 | CA | PHE | A | 371 | 30.242 | 47.615 | 13.005 | 1.00 | 26.36 |
| ATOM | 2765 | C | PHE | A | 371 | 30.828 | 46.295 | 13.463 | 1.00 | 25.97 |
| ATOM | 2766 | O | PHE | A | 371 | 30.118 | 45.320 | 13.638 | 1.00 | 26.84 |
| ATOM | 2767 | CB | PHE | A | 371 | 30.188 | 48.599 | 14.177 | 1.00 | 26.14 |
| ATOM | 2768 | CG | PHE | A | 371 | 29.275 | 48.201 | 15.265 | 1.00 | 27.08 |
| ATOM | 2769 | CD1 | PHE | A | 371 | 29.715 | 47.412 | 16.300 | 1.00 | 30.96 |
| ATOM | 2770 | CD2 | PHE | A | 371 | 27.974 | 48.642 | 15.299 | 1.00 | 25.40 |
| ATOM | 2771 | CE1 | PHE | A | 371 | 28.856 | 47.055 | 17.334 | 1.00 | 26.99 |
| ATOM | 2772 | CE2 | PHE | A | 371 | 27.148 | 48.280 | 16.324 | 1.00 | 24.25 |
| ATOM | 2773 | CZ | PHE | A | 371 | 27.586 | 47.490 | 17.320 | 1.00 | 26.85 |
| ATOM | 2774 | N | TYR | A | 372 | 32.139 | 46.271 | 13.632 | 1.00 | 26.28 |
| ATOM | 2775 | CA | TYR | A | 372 | 32.825 | 45.074 | 14.091 | 1.00 | 26.83 |
| ATOM | 2776 | C | TYR | A | 372 | 33.463 | 45.428 | 15.431 | 1.00 | 28.08 |
| ATOM | 2777 | O | TYR | A | 372 | 33.841 | 46.570 | 15.649 | 1.00 | 28.37 |
| ATOM | 2778 | CB | TYR | A | 372 | 33.879 | 44.638 | 13.069 | 1.00 | 26.10 |
| ATOM | 2779 | CG | TYR | A | 372 | 33.347 | 44.403 | 11.661 | 1.00 | 26.71 |
| ATOM | 2780 | CD1 | TYR | A | 372 | 33.028 | 45.443 | 10.832 | 1.00 | 26.09 |
| ATOM | 2781 | CD2 | TYR | A | 372 | 33.209 | 43.138 | 11.160 | 1.00 | 29.23 |
| ATOM | 2782 | CE1 | TYR | A | 372 | 32.556 | 45.227 | 9.569 | 1.00 | 26.18 |
| ATOM | 2783 | CE2 | TYR | A | 372 | 32.743 | 42.916 | 9.900 | 1.00 | 28.68 |
| ATOM | 2784 | CZ | TYR | A | 372 | 32.424 | 43.958 | 9.117 | 1.00 | 27.90 |
| ATOM | 2785 | OH | TYR | A | 372 | 31.973 | 43.707 | 7.864 | 1.00 | 31.58 |
| ATOM | 2786 | N | LYS | A | 373 | 33.613 | 44.443 | 16.313 | 1.00 | 28.86 |
| ATOM | 2787 | CA | LYS | A | 373 | 34.072 | 44.700 | 17.656 | 1.00 | 29.30 |
| ATOM | 2788 | C | LYS | A | 373 | 34.489 | 43.366 | 18.288 | 1.00 | 29.14 |
| ATOM | 2789 | O | LYS | A | 373 | 33.875 | 42.340 | 18.010 | 1.00 | 29.72 |
| ATOM | 2790 | CB | LYS | A | 373 | 32.880 | 45.336 | 18.386 | 1.00 | 29.76 |
| ATOM | 2791 | CG | LYS | A | 373 | 32.978 | 45.558 | 19.860 | 1.00 | 30.57 |
| ATOM | 2792 | CD | LYS | A | 373 | 31.682 | 46.194 | 20.346 | 1.00 | 32.41 |
| ATOM | 2793 | CE | LYS | A | 373 | 31.844 | 47.024 | 21.596 | 1.00 | 32.07 |
| ATOM | 2794 | NZ | LYS | A | 373 | 32.598 | 46.321 | 22.684 | 1.00 | 29.62 |
| ATOM | 2795 | N | ILE | A | 374 | 35.499 | 43.391 | 19.148 | 1.00 | 28.91 |
| ATOM | 2796 | CA | ILE | A | 374 | 36.021 | 42.191 | 19.781 | 1.00 | 29.20 |
| ATOM | 2797 | C | ILE | A | 374 | 35.148 | 41.887 | 20.984 | 1.00 | 28.30 |
| ATOM | 2798 | O | ILE | A | 374 | 34.898 | 42.761 | 21.774 | 1.00 | 28.69 |
| ATOM | 2799 | CB | ILE | A | 374 | 37.494 | 42.401 | 20.204 | 1.00 | 30.10 |
| ATOM | 2800 | CG1 | ILE | A | 374 | 38.384 | 42.477 | 18.987 | 1.00 | 32.01 |
| ATOM | 2801 | CG2 | ILE | A | 374 | 37.993 | 41.269 | 21.108 | 1.00 | 29.55 |
| ATOM | 2802 | CD1 | ILE | A | 374 | 39.681 | 43.157 | 19.261 | 1.00 | 33.64 |
| ATOM | 2803 | N | ILE | A | 375 | 34.706 | 40.641 | 21.089 | 1.00 | 28.00 |
| ATOM | 2804 | CA | ILE | A | 375 | 33.741 | 40.154 | 22.089 | 1.00 | 28.41 |
| ATOM | 2805 | C | ILE | A | 375 | 34.173 | 38.776 | 22.497 | 1.00 | 27.88 |
| ATOM | 2806 | O | ILE | A | 375 | 34.505 | 37.967 | 21.650 | 1.00 | 26.80 |
| ATOM | 2807 | CB | ILE | A | 375 | 32.327 | 40.001 | 21.440 | 1.00 | 28.32 |
| ATOM | 2808 | CG1 | ILE | A | 375 | 31.805 | 41.337 | 20.929 | 1.00 | 30.15 |
| ATOM | 2809 | CG2 | ILE | A | 375 | 31.333 | 39.397 | 22.409 | 1.00 | 29.72 |
| ATOM | 2810 | CD1 | ILE | A | 375 | 31.377 | 42.302 | 21.983 | 1.00 | 30.99 |
| ATOM | 2811 | N | SER | A | 376 | 34.147 | 38.492 | 23.784 | 1.00 | 27.89 |
| ATOM | 2812 | CA | SER | A | 376 | 34.455 | 37.167 | 24.279 | 1.00 | 29.16 |
| ATOM | 2813 | C | SER | A | 376 | 33.410 | 36.201 | 23.729 | 1.00 | 30.08 |
| ATOM | 2814 | O | SER | A | 376 | 32.236 | 36.458 | 23.875 | 1.00 | 29.10 |
| ATOM | 2815 | CB | SER | A | 376 | 34.383 | 37.165 | 25.822 | 1.00 | 29.45 |
| ATOM | 2816 | OG | SER | A | 376 | 34.900 | 35.961 | 26.326 | 1.00 | 31.37 |
| ATOM | 2817 | N | ASN | A | 377 | 33.814 | 35.100 | 23.102 | 1.00 | 31.00 |
| ATOM | 2818 | CA | ASN | A | 377 | 32.823 | 34.179 | 22.567 | 1.00 | 32.06 |
| ATOM | 2819 | C | ASN | A | 377 | 32.376 | 33.201 | 23.630 | 1.00 | 34.27 |
| ATOM | 2820 | O | ASN | A | 377 | 32.726 | 33.353 | 24.800 | 1.00 | 34.39 |
| ATOM | 2821 | CB | ASN | A | 377 | 33.288 | 33.481 | 21.302 | 1.00 | 32.07 |
| ATOM | 2822 | CG | ASN | A | 377 | 34.414 | 32.523 | 21.538 | 1.00 | 30.43 |
| ATOM | 2823 | OD1 | ASN | A | 377 | 35.098 | 32.122 | 20.596 | 1.00 | 31.65 |
| ATOM | 2824 | ND2 | ASN | A | 377 | 34.622 | 32.151 | 22.767 | 1.00 | 28.95 |
| ATOM | 2825 | N | GLU | A | 378 | 31.595 | 32.205 | 23.245 | 1.00 | 35.46 |
| ATOM | 2826 | CA | GLU | A | 378 | 30.991 | 31.306 | 24.233 | 1.00 | 37.20 |
| ATOM | 2827 | C | GLU | A | 378 | 32.021 | 30.328 | 24.877 | 1.00 | 36.50 |
| ATOM | 2828 | O | GLU | A | 378 | 31.752 | 29.722 | 25.896 | 1.00 | 37.33 |
| ATOM | 2829 | CB | GLU | A | 378 | 29.697 | 30.690 | 23.617 | 1.00 | 37.64 |
| ATOM | 2830 | CG | GLU | A | 378 | 29.425 | 29.208 | 23.836 | 1.00 | 43.49 |
| ATOM | 2831 | CD | GLU | A | 378 | 28.157 | 28.707 | 23.103 | 1.00 | 49.09 |
| ATOM | 2832 | OE1 | GLU | A | 378 | 27.131 | 29.431 | 23.082 | 1.00 | 54.01 |
| ATOM | 2833 | OE2 | GLU | A | 378 | 28.168 | 27.584 | 22.544 | 1.00 | 53.42 |
| ATOM | 2834 | N | GLU | A | 379 | 33.225 | 30.231 | 24.340 | 1.00 | 35.87 |
| ATOM | 2835 | CA | GLU | A | 379 | 34.270 | 29.437 | 24.982 | 1.00 | 36.04 |
| ATOM | 2836 | C | GLU | A | 379 | 35.201 | 30.351 | 25.781 | 1.00 | 34.45 |
| ATOM | 2837 | O | GLU | A | 379 | 36.183 | 29.909 | 26.363 | 1.00 | 34.05 |
| ATOM | 2838 | CB | GLU | A | 379 | 35.131 | 28.688 | 23.957 | 1.00 | 37.56 |
| ATOM | 2839 | CG | GLU | A | 379 | 34.483 | 27.505 | 23.249 | 1.00 | 41.83 |
| ATOM | 2840 | CD | GLU | A | 379 | 33.709 | 27.905 | 22.009 | 1.00 | 48.27 |
| ATOM | 2841 | OE1 | GLU | A | 379 | 34.052 | 28.946 | 21.394 | 1.00 | 50.70 |
| ATOM | 2842 | OE2 | GLU | A | 379 | 32.738 | 27.172 | 21.652 | 1.00 | 53.49 |
| ATOM | 2843 | N | GLY | A | 380 | 34.922 | 31.634 | 25.802 | 1.00 | 32.96 |
| ATOM | 2844 | CA | GLY | A | 380 | 35.759 | 32.536 | 26.570 | 1.00 | 32.06 |
| ATOM | 2845 | C | GLY | A | 380 | 36.963 | 33.130 | 25.827 | 1.00 | 30.76 |
| ATOM | 2846 | O | GLY | A | 380 | 37.865 | 33.636 | 26.488 | 1.00 | 30.06 |
| ATOM | 2847 | N | TYR | A | 381 | 36.942 | 33.106 | 24.490 | 1.00 | 29.35 |
| ATOM | 2848 | CA | TYR | A | 381 | 37.990 | 33.659 | 23.635 | 1.00 | 29.39 |
| ATOM | 2849 | C | TYR | A | 381 | 37.496 | 34.879 | 22.840 | 1.00 | 28.64 |
| ATOM | 2850 | O | TYR | A | 381 | 36.388 | 34.888 | 22.230 | 1.00 | 28.59 |
| ATOM | 2851 | CB | TYR | A | 381 | 38.602 | 32.594 | 22.691 | 1.00 | 30.01 |
| ATOM | 2852 | CG | TYR | A | 381 | 39.328 | 31.479 | 23.441 | 1.00 | 31.34 |
| ATOM | 2853 | CD1 | TYR | A | 381 | 38.625 | 30.401 | 23.962 | 1.00 | 34.09 |
| ATOM | 2854 | CD2 | TYR | A | 381 | 40.698 | 31.535 | 23.677 | 1.00 | 31.70 |
| ATOM | 2855 | CE1 | TYR | A | 381 | 39.258 | 29.401 | 24.666 | 1.00 | 32.67 |
| ATOM | 2856 | CE2 | TYR | A | 381 | 41.338 | 30.526 | 24.368 | 1.00 | 29.86 |
| ATOM | 2857 | CZ | TYR | A | 381 | 40.604 | 29.474 | 24.862 | 1.00 | 32.07 |
| ATOM | 2858 | OH | TYR | A | 381 | 41.210 | 28.462 | 25.564 | 1.00 | 36.44 |
| ATOM | 2859 | N | ARG | A | 382 | 38.341 | 35.900 | 22.839 | 1.00 | 27.18 |
| ATOM | 2860 | CA | ARG | A | 382 | 38.030 | 37.190 | 22.262 | 1.00 | 27.54 |
| ATOM | 2861 | C | ARG | A | 382 | 38.183 | 37.171 | 20.751 | 1.00 | 27.17 |
| ATOM | 2862 | O | ARG | A | 382 | 39.281 | 37.077 | 20.241 | 1.00 | 27.99 |
| ATOM | 2863 | CB | ARG | A | 382 | 38.916 | 38.274 | 22.890 | 1.00 | 27.41 |
| ATOM | 2864 | CG | ARG | A | 382 | 38.377 | 38.750 | 24.239 | 1.00 | 27.74 |
| ATOM | 2865 | CD | ARG | A | 382 | 39.355 | 39.449 | 25.210 | 1.00 | 28.40 |
| ATOM | 2866 | NE | ARG | A | 382 | 38.855 | 39.054 | 26.526 | 1.00 | 27.90 |
| ATOM | 2867 | CZ | ARG | A | 382 | 37.797 | 39.602 | 27.121 | 1.00 | 25.68 |
| ATOM | 2868 | NH1 | ARG | A | 382 | 37.165 | 40.666 | 26.618 | 1.00 | 27.00 |
| ATOM | 2869 | NH2 | ARG | A | 382 | 37.370 | 39.080 | 28.224 | 1.00 | 24.84 |
| ATOM | 2870 | N | HIS | A | 383 | 37.064 | 37.313 | 20.061 | 1.00 | 26.95 |
| ATOM | 2871 | CA | HIS | A | 383 | 37.000 | 37.242 | 18.595 | 1.00 | 25.86 |
| ATOM | 2872 | C | HIS | A | 383 | 36.196 | 38.388 | 18.029 | 1.00 | 26.13 |
| ATOM | 2873 | O | HIS | A | 383 | 35.551 | 39.124 | 18.766 | 1.00 | 26.39 |
| ATOM | 2874 | CB | HIS | A | 383 | 36.436 | 35.902 | 18.170 | 1.00 | 25.61 |
| ATOM | 2875 | CG | HIS | A | 383 | 37.439 | 34.812 | 18.260 | 1.00 | 25.70 |
| ATOM | 2876 | ND1 | HIS | A | 383 | 38.503 | 34.733 | 17.391 | 1.00 | 23.67 |
| ATOM | 2877 | CD2 | HIS | A | 383 | 37.604 | 33.812 | 19.161 | 1.00 | 23.59 |
| ATOM | 2878 | CE1 | HIS | A | 383 | 39.282 | 33.728 | 17.761 | 1.00 | 22.66 |
| ATOM | 2879 | NE2 | HIS | A | 383 | 38.773 | 33.173 | 18.845 | 1.00 | 19.46 |
| ATOM | 2880 | N | ILE | A | 384 | 36.232 | 38.547 | 16.715 | 1.00 | 25.92 |
| ATOM | 2881 | CA | ILE | A | 384 | 35.586 | 39.671 | 16.091 | 1.00 | 26.57 |
| ATOM | 2882 | C | ILE | A | 384 | 34.165 | 39.283 | 15.784 | 1.00 | 27.32 |
| ATOM | 2883 | O | ILE | A | 384 | 33.921 | 38.262 | 15.141 | 1.00 | 27.14 |
| ATOM | 2884 | CB | ILE | A | 384 | 36.306 | 40.039 | 14.778 | 1.00 | 26.65 |
| ATOM | 2885 | CG1 | ILE | A | 384 | 37.734 | 40.458 | 15.054 | 1.00 | 27.00 |
| ATOM | 2886 | CG2 | ILE | A | 384 | 35.552 | 41.135 | 14.042 | 1.00 | 26.58 |
| ATOM | 2887 | CD1 | ILE | A | 384 | 38.700 | 40.235 | 13.849 | 1.00 | 29.40 |
| ATOM | 2888 | N | CYS | A | 385 | 33.238 | 40.114 | 16.222 | 1.00 | 27.67 |
| ATOM | 2889 | CA | CYS | A | 385 | 31.854 | 39.885 | 15.968 | 1.00 | 28.87 |
| ATOM | 2890 | C | CYS | A | 385 | 31.428 | 41.007 | 15.069 | 1.00 | 27.76 |
| ATOM | 2891 | O | CYS | A | 385 | 31.951 | 42.109 | 15.186 | 1.00 | 27.14 |
| ATOM | 2892 | CB | CYS | A | 385 | 31.028 | 39.905 | 17.272 | 1.00 | 29.48 |
| ATOM | 2893 | SG | CYS | A | 385 | 29.828 | 38.587 | 17.270 | 1.00 | 37.72 |
| ATOM | 2894 | N | TYR | A | 386 | 30.466 | 40.709 | 14.189 | 1.00 | 27.09 |
| ATOM | 2895 | CA | TYR | A | 386 | 29.895 | 41.657 | 13.260 | 1.00 | 27.30 |
| ATOM | 2896 | C | TYR | A | 386 | 28.467 | 41.893 | 13.698 | 1.00 | 27.66 |
| ATOM | 2897 | O | TYR | A | 386 | 27.728 | 40.921 | 13.954 | 1.00 | 29.00 |
| ATOM | 2898 | CB | TYR | A | 386 | 29.936 | 41.065 | 11.870 | 1.00 | 27.12 |
| ATOM | 2899 | CG | TYR | A | 386 | 29.181 | 41.792 | 10.797 | 1.00 | 26.46 |
| ATOM | 2900 | CD1 | TYR | A | 386 | 29.530 | 43.057 | 10.410 | 1.00 | 22.92 |
| ATOM | 2901 | CD2 | TYR | A | 386 | 28.133 | 41.158 | 10.124 | 1.00 | 27.21 |
| ATOM | 2902 | CE1 | TYR | A | 386 | 28.844 | 43.692 | 9.400 | 1.00 | 26.58 |
| ATOM | 2903 | CE2 | TYR | A | 386 | 27.428 | 41.801 | 9.142 | 1.00 | 27.27 |
| ATOM | 2904 | CZ | TYR | A | 386 | 27.788 | 43.055 | 8.773 | 1.00 | 26.83 |
| ATOM | 2905 | OH | TYR | A | 386 | 27.075 | 43.689 | 7.788 | 1.00 | 29.03 |
| ATOM | 2906 | N | PHE | A | 387 | 28.110 | 43.172 | 13.812 | 1.00 | 27.27 |
| ATOM | 2907 | CA | PHE | A | 387 | 26.840 | 43.648 | 14.343 | 1.00 | 27.38 |
| ATOM | 2908 | C | PHE | A | 387 | 26.159 | 44.523 | 13.298 | 1.00 | 27.63 |
| ATOM | 2909 | O | PHE | A | 387 | 26.830 | 45.404 | 12.719 | 1.00 | 27.68 |
| ATOM | 2910 | CB | PHE | A | 387 | 27.100 | 44.625 | 15.526 | 1.00 | 27.34 |
| ATOM | 2911 | CG | PHE | A | 387 | 27.511 | 43.969 | 16.806 | 1.00 | 28.85 |
| ATOM | 2912 | CD1 | PHE | A | 387 | 28.871 | 43.725 | 17.092 | 1.00 | 31.74 |
| ATOM | 2913 | CD2 | PHE | A | 387 | 26.575 | 43.626 | 17.750 | 1.00 | 28.35 |
| ATOM | 2914 | CE1 | PHE | A | 387 | 29.245 | 43.137 | 18.294 | 1.00 | 29.31 |
| ATOM | 2915 | CE2 | PHE | A | 387 | 26.965 | 43.056 | 18.952 | 1.00 | 30.57 |
| ATOM | 2916 | CZ | PHE | A | 387 | 28.294 | 42.800 | 19.210 | 1.00 | 30.81 |
| ATOM | 2917 | N | GLN | A | 388 | 24.852 | 44.352 | 13.101 | 1.00 | 27.28 |
| ATOM | 2918 | CA | GLN | A | 388 | 24.102 | 45.249 | 12.225 | 1.00 | 28.29 |
| ATOM | 2919 | C | GLN | A | 388 | 23.250 | 46.113 | 13.095 | 1.00 | 28.91 |
| ATOM | 2920 | O | GLN | A | 388 | 22.579 | 45.663 | 13.993 | 1.00 | 29.61 |
| ATOM | 2921 | CB | GLN | A | 388 | 23.320 | 44.493 | 11.152 | 1.00 | 27.95 |
| ATOM | 2922 | CG | GLN | A | 388 | 24.261 | 43.652 | 10.242 | 1.00 | 29.84 |
| ATOM | 2923 | CD | GLN | A | 388 | 23.520 | 42.916 | 9.116 | 1.00 | 30.76 |
| ATOM | 2924 | OE1 | GLN | A | 388 | 22.495 | 42.320 | 9.359 | 1.00 | 32.29 |
| ATOM | 2925 | NE2 | GLN | A | 388 | 24.056 | 42.966 | 7.896 | 1.00 | 32.24 |
| ATOM | 2926 | N | ILE | A | 389 | 23.244 | 47.385 | 12.822 | 1.00 | 31.80 |
| ATOM | 2927 | CA | ILE | A | 389 | 22.690 | 48.323 | 13.800 | 1.00 | 34.40 |
| ATOM | 2928 | C | ILE | A | 389 | 21.329 | 48.154 | 14.439 | 1.00 | 36.24 |
| ATOM | 2929 | O | ILE | A | 389 | 21.186 | 48.523 | 15.619 | 1.00 | 39.05 |
| ATOM | 2930 | CB | ILE | A | 389 | 22.894 | 49.752 | 13.337 | 1.00 | 34.30 |
| ATOM | 2931 | CG1 | ILE | A | 389 | 24.267 | 50.161 | 13.838 | 1.00 | 35.49 |
| ATOM | 2932 | CG2 | ILE | A | 389 | 21.840 | 50.662 | 13.911 | 1.00 | 35.63 |
| ATOM | 2933 | CD1 | ILE | A | 389 | 24.667 | 51.476 | 13.397 | 1.00 | 37.16 |
| ATOM | 2934 | N | ASP | A | 390 | 20.342 | 47.603 | 13.764 | 1.00 | 37.12 |
| ATOM | 2935 | CA | ASP | A | 390 | 19.049 | 47.422 | 14.446 | 1.00 | 38.19 |
| ATOM | 2936 | C | ASP | A | 390 | 18.648 | 45.969 | 14.536 | 1.00 | 37.79 |
| ATOM | 2937 | O | ASP | A | 390 | 17.470 | 45.665 | 14.668 | 1.00 | 36.89 |
| ATOM | 2938 | CB | ASP | A | 390 | 17.920 | 48.129 | 13.674 | 1.00 | 38.61 |
| ATOM | 2939 | CG | ASP | A | 390 | 17.817 | 49.577 | 14.029 | 1.00 | 41.07 |
| ATOM | 2940 | OD1 | ASP | A | 390 | 17.580 | 49.835 | 15.218 | 1.00 | 45.39 |
| ATOM | 2941 | OD2 | ASP | A | 390 | 17.971 | 50.513 | 13.212 | 1.00 | 43.05 |
| ATOM | 2942 | N | LYS | A | 391 | 19.622 | 45.083 | 14.434 | 1.00 | 36.90 |
| ATOM | 2943 | CA | LYS | A | 391 | 19.362 | 43.655 | 14.372 | 1.00 | 36.88 |
| ATOM | 2944 | C | LYS | A | 391 | 19.915 | 43.078 | 15.622 | 1.00 | 35.55 |
| ATOM | 2945 | O | LYS | A | 391 | 20.926 | 43.539 | 16.121 | 1.00 | 33.43 |
| ATOM | 2946 | CB | LYS | A | 391 | 20.103 | 43.015 | 13.160 | 1.00 | 37.29 |
| ATOM | 2947 | CG | LYS | A | 391 | 19.262 | 42.731 | 11.901 | 1.00 | 41.18 |
| ATOM | 2948 | CD | LYS | A | 391 | 20.093 | 41.805 | 10.904 | 1.00 | 45.70 |
| ATOM | 2949 | CE | LYS | A | 391 | 19.397 | 41.478 | 9.550 | 1.00 | 45.90 |
| ATOM | 2950 | NZ | LYS | A | 391 | 20.375 | 41.390 | 8.330 | 1.00 | 46.25 |
| ATOM | 2951 | N | LYS | A | 392 | 19.278 | 42.036 | 16.103 | 1.00 | 35.58 |
| ATOM | 2952 | CA | LYS | A | 392 | 19.758 | 41.339 | 17.270 | 1.00 | 36.06 |
| ATOM | 2953 | C | LYS | A | 392 | 21.064 | 40.596 | 17.002 | 1.00 | 36.18 |
| ATOM | 2954 | O | LYS | A | 392 | 21.482 | 40.424 | 15.839 | 1.00 | 35.91 |
| ATOM | 2955 | CB | LYS | A | 392 | 18.722 | 40.300 | 17.661 | 1.00 | 36.65 |
| ATOM | 2956 | CG | LYS | A | 392 | 18.442 | 39.262 | 16.551 | 1.00 | 38.84 |
| ATOM | 2957 | CD | LYS | A | 392 | 17.712 | 38.042 | 17.122 | 1.00 | 41.45 |
| ATOM | 2958 | CE | LYS | A | 392 | 16.893 | 37.290 | 16.095 | 1.00 | 41.02 |
| ATOM | 2959 | NZ | LYS | A | 392 | 16.224 | 36.066 | 16.691 | 1.00 | 38.35 |
| ATOM | 2960 | N | ASP | A | 393 | 21.667 | 40.138 | 18.099 | 1.00 | 35.78 |
| ATOM | 2961 | CA | ASP | A | 393 | 22.823 | 39.268 | 18.103 | 1.00 | 35.76 |
| ATOM | 2962 | C | ASP | A | 393 | 23.968 | 39.817 | 17.239 | 1.00 | 34.85 |
| ATOM | 2963 | O | ASP | A | 393 | 23.914 | 40.946 | 16.741 | 1.00 | 33.92 |
| ATOM | 2964 | CB | ASP | A | 393 | 22.446 | 37.857 | 17.584 | 1.00 | 36.56 |
| ATOM | 2965 | CG | ASP | A | 393 | 21.231 | 37.219 | 18.295 | 1.00 | 40.35 |
| ATOM | 2966 | OD1 | ASP | A | 393 | 21.026 | 37.404 | 19.531 | 1.00 | 42.98 |
| ATOM | 2967 | OD2 | ASP | A | 393 | 20.435 | 36.460 | 17.672 | 1.00 | 42.96 |
| ATOM | 2968 | N | CYS | A | 394 | 25.019 | 39.019 | 17.088 | 1.00 | 33.41 |
| ATOM | 2969 | CA | CYS | A | 394 | 26.088 | 39.369 | 16.187 | 1.00 | 33.82 |
| ATOM | 2970 | C | CYS | A | 394 | 26.580 | 38.083 | 15.570 | 1.00 | 32.97 |
| ATOM | 2971 | O | CYS | A | 394 | 26.256 | 36.967 | 16.039 | 1.00 | 32.97 |
| ATOM | 2972 | CB | CYS | A | 394 | 27.238 | 40.084 | 16.914 | 1.00 | 34.46 |
| ATOM | 2973 | SG | CYS | A | 394 | 28.009 | 39.022 | 18.136 | 1.00 | 38.67 |
| ATOM | 2974 | N | THR | A | 395 | 27.388 | 38.231 | 14.531 | 1.00 | 30.95 |
| ATOM | 2975 | CA | THR | A | 395 | 27.928 | 37.081 | 13.851 | 1.00 | 29.51 |
| ATOM | 2976 | C | THR | A | 395 | 29.434 | 37.066 | 14.088 | 1.00 | 28.32 |
| ATOM | 2977 | O | THR | A | 395 | 30.128 | 38.006 | 13.729 | 1.00 | 26.38 |
| ATOM | 2978 | CB | THR | A | 395 | 27.676 | 37.235 | 12.342 | 1.00 | 30.00 |
| ATOM | 2979 | OG1 | THR | A | 395 | 26.271 | 37.381 | 12.063 | 1.00 | 30.37 |
| ATOM | 2980 | CG2 | THR | A | 395 | 28.140 | 36.013 | 11.558 | 1.00 | 28.82 |
| ATOM | 2981 | N | PHE | A | 396 | 29.935 | 36.000 | 14.682 | 1.00 | 27.62 |
| ATOM | 2982 | CA | PHE | A | 396 | 31.356 | 35.805 | 14.830 | 1.00 | 27.77 |
| ATOM | 2983 | C | PHE | A | 396 | 32.016 | 35.496 | 13.486 | 1.00 | 28.57 |
| ATOM | 2984 | O | PHE | A | 396 | 31.582 | 34.611 | 12.753 | 1.00 | 29.01 |
| ATOM | 2985 | CB | PHE | A | 396 | 31.601 | 34.686 | 15.818 | 1.00 | 27.37 |
| ATOM | 2986 | CG | PHE | A | 396 | 31.567 | 35.149 | 17.238 | 1.00 | 28.76 |
| ATOM | 2987 | CD1 | PHE | A | 396 | 32.505 | 36.041 | 17.691 | 1.00 | 27.09 |
| ATOM | 2988 | CD2 | PHE | A | 396 | 30.540 | 34.760 | 18.091 | 1.00 | 29.92 |
| ATOM | 2989 | CE1 | PHE | A | 396 | 32.475 | 36.506 | 19.012 | 1.00 | 30.67 |
| ATOM | 2990 | CE2 | PHE | A | 396 | 30.488 | 35.228 | 19.392 | 1.00 | 30.26 |
| ATOM | 2991 | CZ | PHE | A | 396 | 31.451 | 36.112 | 19.855 | 1.00 | 31.58 |
| ATOM | 2992 | N | ILE | A | 397 | 33.018 | 36.271 | 13.127 | 1.00 | 29.00 |
| ATOM | 2993 | CA | ILE | A | 397 | 33.729 | 36.054 | 11.878 | 1.00 | 29.41 |
| ATOM | 2994 | C | ILE | A | 397 | 35.123 | 35.411 | 12.098 | 1.00 | 29.48 |
| ATOM | 2995 | O | ILE | A | 397 | 35.741 | 34.942 | 11.151 | 1.00 | 29.78 |
| ATOM | 2996 | CB | ILE | A | 397 | 33.821 | 37.354 | 11.057 | 1.00 | 29.93 |
| ATOM | 2997 | CG1 | ILE | A | 397 | 34.591 | 38.407 | 11.809 | 1.00 | 29.25 |
| ATOM | 2998 | CG2 | ILE | A | 397 | 32.457 | 37.835 | 10.718 | 1.00 | 29.75 |
| ATOM | 2999 | CD1 | ILE | A | 397 | 35.269 | 39.402 | 10.924 | 1.00 | 31.20 |
| ATOM | 3000 | N | THR | A | 398 | 35.589 | 35.367 | 13.339 | 1.00 | 29.02 |
| ATOM | 3001 | CA | THR | A | 398 | 36.754 | 34.565 | 13.695 | 1.00 | 29.32 |
| ATOM | 3002 | C | THR | A | 398 | 36.380 | 33.686 | 14.866 | 1.00 | 29.68 |
| ATOM | 3003 | O | THR | A | 398 | 35.436 | 33.977 | 15.590 | 1.00 | 29.46 |
| ATOM | 3004 | CB | THR | A | 398 | 37.963 | 35.412 | 14.114 | 1.00 | 29.68 |
| ATOM | 3005 | OG1 | THR | A | 398 | 37.645 | 36.178 | 15.304 | 1.00 | 29.00 |
| ATOM | 3006 | CG2 | THR | A | 398 | 38.339 | 36.415 | 13.020 | 1.00 | 28.51 |
| ATOM | 3007 | N | LYS | A | 399 | 37.143 | 32.629 | 15.074 | 1.00 | 30.35 |
| ATOM | 3008 | CA | LYS | A | 399 | 36.885 | 31.683 | 16.156 | 1.00 | 32.18 |
| ATOM | 3009 | C | LYS | A | 399 | 38.149 | 30.867 | 16.321 | 1.00 | 30.94 |
| ATOM | 3010 | O | LYS | A | 399 | 38.960 | 30.812 | 15.422 | 1.00 | 30.64 |
| ATOM | 3011 | CB | LYS | A | 399 | 35.741 | 30.716 | 15.802 | 1.00 | 33.03 |
| ATOM | 3012 | CG | LYS | A | 399 | 34.969 | 31.118 | 14.547 | 1.00 | 39.43 |
| ATOM | 3013 | CD | LYS | A | 399 | 33.751 | 30.221 | 14.171 | 1.00 | 44.29 |
| ATOM | 3014 | CE | LYS | A | 399 | 32.554 | 31.100 | 13.683 | 1.00 | 45.24 |
| ATOM | 3015 | NZ | LYS | A | 399 | 31.425 | 30.316 | 13.063 | 1.00 | 48.22 |
| ATOM | 3016 | N | GLY | A | 400 | 38.297 | 30.231 | 17.468 | 1.00 | 30.34 |
| ATOM | 3017 | CA | GLY | A | 400 | 39.430 | 29.389 | 17.734 | 1.00 | 30.41 |
| ATOM | 3018 | C | GLY | A | 400 | 39.998 | 29.650 | 19.130 | 1.00 | 31.49 |
| ATOM | 3019 | O | GLY | A | 400 | 39.655 | 30.639 | 19.811 | 1.00 | 29.63 |
| ATOM | 3020 | N | THR | A | 401 | 40.858 | 28.738 | 19.558 | 1.00 | 31.81 |
| ATOM | 3021 | CA | THR | A | 401 | 41.543 | 28.879 | 20.828 | 1.00 | 34.07 |
| ATOM | 3022 | C | THR | A | 401 | 42.763 | 29.832 | 20.740 | 1.00 | 32.71 |
| ATOM | 3023 | O | THR | A | 401 | 43.892 | 29.401 | 20.778 | 1.00 | 34.00 |
| ATOM | 3024 | CB | THR | A | 401 | 41.978 | 27.494 | 21.314 | 1.00 | 34.50 |
| ATOM | 3025 | OG1 | THR | A | 401 | 40.812 | 26.676 | 21.574 | 1.00 | 39.26 |
| ATOM | 3026 | CG2 | THR | A | 401 | 42.610 | 27.608 | 22.641 | 1.00 | 36.18 |
| ATOM | 3027 | N | TRP | A | 402 | 42.508 | 31.126 | 20.642 | 1.00 | 30.65 |
| ATOM | 3028 | CA | TRP | A | 402 | 43.548 | 32.138 | 20.576 | 1.00 | 28.93 |
| ATOM | 3029 | C | TRP | A | 402 | 42.723 | 33.392 | 20.565 | 1.00 | 27.54 |
| ATOM | 3030 | O | TRP | A | 402 | 41.507 | 33.307 | 20.544 | 1.00 | 25.60 |
| ATOM | 3031 | CB | TRP | A | 402 | 44.383 | 32.037 | 19.298 | 1.00 | 28.72 |
| ATOM | 3032 | CG | TRP | A | 402 | 43.607 | 31.774 | 18.069 | 1.00 | 29.95 |
| ATOM | 3033 | CD1 | TRP | A | 402 | 43.274 | 30.538 | 17.553 | 1.00 | 31.24 |
| ATOM | 3034 | CD2 | TRP | A | 402 | 43.058 | 32.735 | 17.160 | 1.00 | 30.14 |
| ATOM | 3035 | NE1 | TRP | A | 402 | 42.541 | 30.686 | 16.405 | 1.00 | 29.78 |
| ATOM | 3036 | CE2 | TRP | A | 402 | 42.397 | 32.017 | 16.133 | 1.00 | 30.91 |
| ATOM | 3037 | CE3 | TRP | A | 402 | 43.042 | 34.122 | 17.113 | 1.00 | 30.21 |
| ATOM | 3038 | CZ2 | TRP | A | 402 | 41.723 | 32.643 | 15.094 | 1.00 | 30.88 |
| ATOM | 3039 | CZ3 | TRP | A | 402 | 42.385 | 34.744 | 16.073 | 1.00 | 31.74 |
| ATOM | 3040 | CH2 | TRP | A | 402 | 41.730 | 34.008 | 15.077 | 1.00 | 30.37 |
| ATOM | 3041 | N | GLU | A | 403 | 43.325 | 34.564 | 20.491 | 1.00 | 27.23 |
| ATOM | 3042 | CA | GLU | A | 403 | 42.468 | 35.748 | 20.447 | 1.00 | 26.73 |
| ATOM | 3043 | C | GLU | A | 403 | 42.904 | 36.792 | 19.498 | 1.00 | 25.83 |
| ATOM | 3044 | O | GLU | A | 403 | 44.103 | 36.972 | 19.227 | 1.00 | 24.50 |
| ATOM | 3045 | CB | GLU | A | 403 | 42.383 | 36.461 | 21.830 | 1.00 | 27.45 |
| ATOM | 3046 | CG | GLU | A | 403 | 42.176 | 35.552 | 23.036 | 1.00 | 28.63 |
| ATOM | 3047 | CD | GLU | A | 403 | 41.798 | 36.314 | 24.299 | 1.00 | 31.80 |
| ATOM | 3048 | OE1 | GLU | A | 403 | 42.453 | 37.326 | 24.598 | 1.00 | 30.89 |
| ATOM | 3049 | OE2 | GLU | A | 403 | 40.842 | 35.882 | 24.992 | 1.00 | 36.19 |
| ATOM | 3050 | N | VAL | A | 404 | 41.899 | 37.558 | 19.077 | 1.00 | 25.24 |
| ATOM | 3051 | CA | VAL | A | 404 | 42.127 | 38.736 | 18.314 | 1.00 | 24.95 |
| ATOM | 3052 | C | VAL | A | 404 | 42.569 | 39.806 | 19.296 | 1.00 | 26.20 |
| ATOM | 3053 | O | VAL | A | 404 | 41.944 | 39.991 | 20.353 | 1.00 | 25.92 |
| ATOM | 3054 | CB | VAL | A | 404 | 40.908 | 39.172 | 17.633 | 1.00 | 24.47 |
| ATOM | 3055 | CG1 | VAL | A | 404 | 41.217 | 40.371 | 16.843 | 1.00 | 26.01 |
| ATOM | 3056 | CG2 | VAL | A | 404 | 40.379 | 38.055 | 16.711 | 1.00 | 25.01 |
| ATOM | 3057 | N | ILE | A | 405 | 43.646 | 40.501 | 18.960 | 1.00 | 26.33 |
| ATOM | 3058 | CA | ILE | A | 405 | 44.216 | 41.481 | 19.848 | 1.00 | 27.21 |
| ATOM | 3059 | C | ILE | A | 405 | 43.658 | 42.828 | 19.568 | 1.00 | 26.94 |
| ATOM | 3060 | O | ILE | A | 405 | 43.341 | 43.569 | 20.467 | 1.00 | 27.10 |
| ATOM | 3061 | CB | ILE | A | 405 | 45.726 | 41.499 | 19.685 | 1.00 | 28.08 |
| ATOM | 3062 | CG1 | ILE | A | 405 | 46.290 | 40.270 | 20.389 | 1.00 | 29.19 |
| ATOM | 3063 | CG2 | ILE | A | 405 | 46.301 | 42.773 | 20.334 | 1.00 | 29.53 |
| ATOM | 3064 | CD1 | ILE | A | 405 | 47.238 | 39.565 | 19.592 | 1.00 | 32.57 |
| ATOM | 3065 | N | GLY | A | 406 | 43.505 | 43.133 | 18.299 | 1.00 | 26.78 |
| ATOM | 3066 | CA | GLY | A | 406 | 42.928 | 44.393 | 17.909 | 1.00 | 26.62 |
| ATOM | 3067 | C | GLY | A | 406 | 42.486 | 44.334 | 16.460 | 1.00 | 26.29 |
| ATOM | 3068 | O | GLY | A | 406 | 43.010 | 43.541 | 15.673 | 1.00 | 26.04 |
| ATOM | 3069 | N | ILE | A | 407 | 41.493 | 45.141 | 16.126 | 1.00 | 25.77 |
| ATOM | 3070 | CA | ILE | A | 407 | 41.097 | 45.320 | 14.747 | 1.00 | 27.03 |
| ATOM | 3071 | C | ILE | A | 407 | 41.827 | 46.578 | 14.245 | 1.00 | 27.04 |
| ATOM | 3072 | O | ILE | A | 407 | 41.705 | 47.609 | 14.874 | 1.00 | 25.76 |
| ATOM | 3073 | CB | ILE | A | 407 | 39.593 | 45.587 | 14.689 | 1.00 | 26.94 |
| ATOM | 3074 | CG1 | ILE | A | 407 | 38.838 | 44.334 | 15.103 | 1.00 | 27.00 |
| ATOM | 3075 | CG2 | ILE | A | 407 | 39.227 | 46.096 | 13.298 | 1.00 | 26.67 |
| ATOM | 3076 | CD1 | ILE | A | 407 | 37.368 | 44.598 | 15.531 | 1.00 | 27.52 |
| ATOM | 3077 | N | GLU | A | 408 | 42.507 | 46.509 | 13.099 | 1.00 | 27.34 |
| ATOM | 3078 | CA | GLU | A | 408 | 43.382 | 47.617 | 12.656 | 1.00 | 27.57 |
| ATOM | 3079 | C | GLU | A | 408 | 42.929 | 48.440 | 11.473 | 1.00 | 27.19 |
| ATOM | 3080 | O | GLU | A | 408 | 43.285 | 49.590 | 11.350 | 1.00 | 26.59 |
| ATOM | 3081 | CB | GLU | A | 408 | 44.748 | 47.060 | 12.328 | 1.00 | 27.48 |
| ATOM | 3082 | CG | GLU | A | 408 | 45.292 | 46.188 | 13.431 | 1.00 | 28.71 |
| ATOM | 3083 | CD | GLU | A | 408 | 45.563 | 46.968 | 14.691 | 1.00 | 29.19 |
| ATOM | 3084 | OE1 | GLU | A | 408 | 46.036 | 48.123 | 14.625 | 1.00 | 33.20 |
| ATOM | 3085 | OE2 | GLU | A | 408 | 45.311 | 46.411 | 15.740 | 1.00 | 32.03 |
| ATOM | 3086 | N | ALA | A | 409 | 42.171 | 47.829 | 10.583 | 1.00 | 27.17 |
| ATOM | 3087 | CA | ALA | A | 409 | 41.609 | 48.561 | 9.464 | 1.00 | 27.43 |
| ATOM | 3088 | C | ALA | A | 409 | 40.409 | 47.836 | 8.841 | 1.00 | 28.16 |
| ATOM | 3089 | O | ALA | A | 409 | 40.258 | 46.600 | 8.909 | 1.00 | 27.79 |
| ATOM | 3090 | CB | ALA | A | 409 | 42.649 | 48.820 | 8.448 | 1.00 | 26.95 |
| ATOM | 3091 | N | LEU | A | 410 | 39.587 | 48.625 | 8.187 | 1.00 | 29.93 |
| ATOM | 3092 | CA | LEU | A | 410 | 38.356 | 48.130 | 7.595 | 1.00 | 31.53 |
| ATOM | 3093 | C | LEU | A | 410 | 38.122 | 48.796 | 6.276 | 1.00 | 31.83 |
| ATOM | 3094 | O | LEU | A | 410 | 38.068 | 50.000 | 6.214 | 1.00 | 30.83 |
| ATOM | 3095 | CB | LEU | A | 410 | 37.213 | 48.493 | 8.515 | 1.00 | 31.79 |
| ATOM | 3096 | CG | LEU | A | 410 | 35.865 | 47.763 | 8.429 | 1.00 | 34.75 |
| ATOM | 3097 | CD1 | LEU | A | 410 | 34.773 | 48.737 | 8.020 | 1.00 | 34.78 |
| ATOM | 3098 | CD2 | LEU | A | 410 | 35.860 | 46.511 | 7.533 | 1.00 | 34.05 |
| ATOM | 3099 | N | THR | A | 411 | 38.011 | 48.011 | 5.213 | 1.00 | 33.31 |
| ATOM | 3100 | CA | THR | A | 411 | 37.615 | 48.548 | 3.918 | 1.00 | 34.38 |
| ATOM | 3101 | C | THR | A | 411 | 36.421 | 47.729 | 3.500 | 1.00 | 34.97 |
| ATOM | 3102 | O | THR | A | 411 | 36.029 | 46.811 | 4.222 | 1.00 | 34.40 |
| ATOM | 3103 | CB | THR | A | 411 | 38.706 | 48.343 | 2.869 | 1.00 | 34.65 |
| ATOM | 3104 | OG1 | THR | A | 411 | 38.923 | 46.934 | 2.726 | 1.00 | 35.94 |
| ATOM | 3105 | CG2 | THR | A | 411 | 40.014 | 48.894 | 3.335 | 1.00 | 35.71 |
| ATOM | 3106 | N | SER | A | 412 | 35.905 | 48.016 | 2.299 | 1.00 | 36.01 |
| ATOM | 3107 | CA | SER | A | 412 | 34.772 | 47.309 | 1.697 | 1.00 | 36.64 |
| ATOM | 3108 | C | SER | A | 412 | 34.877 | 45.792 | 1.670 | 1.00 | 36.51 |
| ATOM | 3109 | O | SER | A | 412 | 33.891 | 45.081 | 1.952 | 1.00 | 37.08 |
| ATOM | 3110 | CB | SER | A | 412 | 34.616 | 47.740 | 0.231 | 1.00 | 37.15 |
| ATOM | 3111 | OG | SER | A | 412 | 34.192 | 49.076 | 0.121 | 1.00 | 38.14 |
| ATOM | 3112 | N | ASP | A | 413 | 36.054 | 45.322 | 1.275 | 1.00 | 35.36 |
| ATOM | 3113 | CA | ASP | A | 413 | 36.320 | 43.913 | 1.011 | 1.00 | 36.07 |
| ATOM | 3114 | C | ASP | A | 413 | 37.089 | 43.196 | 2.097 | 1.00 | 34.65 |
| ATOM | 3115 | O | ASP | A | 413 | 37.116 | 41.966 | 2.130 | 1.00 | 34.82 |
| ATOM | 3116 | CB | ASP | A | 413 | 37.122 | 43.802 | −0.290 | 1.00 | 36.80 |
| ATOM | 3117 | CG | ASP | A | 413 | 36.506 | 44.618 | −1.427 | 1.00 | 40.03 |
| ATOM | 3118 | OD1 | ASP | A | 413 | 35.273 | 44.524 | −1.685 | 1.00 | 42.94 |
| ATOM | 3119 | OD2 | ASP | A | 413 | 37.176 | 45.405 | −2.109 | 1.00 | 43.99 |
| ATOM | 3120 | N | TYR | A | 414 | 37.740 | 43.946 | 2.977 | 1.00 | 33.46 |
| ATOM | 3121 | CA | TYR | A | 414 | 38.603 | 43.310 | 3.950 | 1.00 | 32.77 |
| ATOM | 3122 | C | TYR | A | 414 | 38.612 | 43.975 | 5.315 | 1.00 | 30.84 |
| ATOM | 3123 | O | TYR | A | 414 | 38.589 | 45.206 | 5.422 | 1.00 | 31.66 |
| ATOM | 3124 | CB | TYR | A | 414 | 40.051 | 43.363 | 3.422 | 1.00 | 33.72 |
| ATOM | 3125 | CG | TYR | A | 414 | 40.316 | 42.543 | 2.178 | 1.00 | 36.47 |
| ATOM | 3126 | CD1 | TYR | A | 414 | 40.564 | 41.183 | 2.273 | 1.00 | 40.26 |
| ATOM | 3127 | CD2 | TYR | A | 414 | 40.369 | 43.137 | 0.915 | 1.00 | 37.42 |
| ATOM | 3128 | CE1 | TYR | A | 414 | 40.822 | 40.427 | 1.159 | 1.00 | 39.48 |
| ATOM | 3129 | CE2 | TYR | A | 414 | 40.629 | 42.385 | −0.221 | 1.00 | 38.74 |
| ATOM | 3130 | CZ | TYR | A | 414 | 40.848 | 41.021 | −0.086 | 1.00 | 40.96 |
| ATOM | 3131 | OH | TYR | A | 414 | 41.112 | 40.217 | −1.184 | 1.00 | 45.05 |
| ATOM | 3132 | N | LEU | A | 415 | 38.613 | 43.155 | 6.351 | 1.00 | 28.44 |
| ATOM | 3133 | CA | LEU | A | 415 | 38.906 | 43.631 | 7.682 | 1.00 | 27.58 |
| ATOM | 3134 | C | LEU | A | 415 | 40.332 | 43.153 | 8.016 | 1.00 | 27.60 |
| ATOM | 3135 | O | LEU | A | 415 | 40.671 | 41.994 | 7.804 | 1.00 | 26.61 |
| ATOM | 3136 | CB | LEU | A | 415 | 37.923 | 43.082 | 8.698 | 1.00 | 27.16 |
| ATOM | 3137 | CG | LEU | A | 415 | 38.089 | 43.643 | 10.096 | 1.00 | 26.32 |
| ATOM | 3138 | CD1 | LEU | A | 415 | 36.785 | 43.538 | 10.868 | 1.00 | 26.01 |
| ATOM | 3139 | CD2 | LEU | A | 415 | 39.212 | 42.913 | 10.798 | 1.00 | 24.32 |
| ATOM | 3140 | N | TYR | A | 416 | 41.169 | 44.047 | 8.541 | 1.00 | 27.81 |
| ATOM | 3141 | CA | TYR | A | 416 | 42.513 | 43.648 | 8.909 | 1.00 | 27.67 |
| ATOM | 3142 | C | TYR | A | 416 | 42.594 | 43.578 | 10.447 | 1.00 | 27.84 |
| ATOM | 3143 | O | TYR | A | 416 | 41.948 | 44.334 | 11.150 | 1.00 | 28.65 |
| ATOM | 3144 | CB | TYR | A | 416 | 43.530 | 44.631 | 8.325 | 1.00 | 27.67 |
| ATOM | 3145 | CG | TYR | A | 416 | 43.603 | 44.705 | 6.801 | 1.00 | 26.27 |
| ATOM | 3146 | CD1 | TYR | A | 416 | 42.648 | 45.398 | 6.065 | 1.00 | 24.50 |
| ATOM | 3147 | CD2 | TYR | A | 416 | 44.655 | 44.110 | 6.103 | 1.00 | 26.34 |
| ATOM | 3148 | CE1 | TYR | A | 416 | 42.734 | 45.514 | 4.695 | 1.00 | 26.01 |
| ATOM | 3149 | CE2 | TYR | A | 416 | 44.730 | 44.204 | 4.747 | 1.00 | 26.06 |
| ATOM | 3150 | CZ | TYR | A | 416 | 43.772 | 44.914 | 4.046 | 1.00 | 26.01 |
| ATOM | 3151 | OH | TYR | A | 416 | 43.840 | 44.993 | 2.696 | 1.00 | 34.51 |
| ATOM | 3152 | N | TYR | A | 417 | 43.374 | 42.651 | 10.976 | 1.00 | 27.82 |
| ATOM | 3153 | CA | TYR | A | 417 | 43.477 | 42.514 | 12.420 | 1.00 | 26.63 |
| ATOM | 3154 | C | TYR | A | 417 | 44.745 | 41.820 | 12.866 | 1.00 | 26.21 |
| ATOM | 3155 | O | TYR | A | 417 | 45.447 | 41.191 | 12.063 | 1.00 | 24.79 |
| ATOM | 3156 | CB | TYR | A | 417 | 42.258 | 41.763 | 12.991 | 1.00 | 26.04 |
| ATOM | 3157 | CG | TYR | A | 417 | 42.222 | 40.301 | 12.668 | 1.00 | 24.88 |
| ATOM | 3158 | CD1 | TYR | A | 417 | 41.636 | 39.838 | 11.495 | 1.00 | 26.75 |
| ATOM | 3159 | CD2 | TYR | A | 417 | 42.739 | 39.372 | 13.549 | 1.00 | 22.61 |
| ATOM | 3160 | CE1 | TYR | A | 417 | 41.588 | 38.450 | 11.216 | 1.00 | 25.54 |
| ATOM | 3162 | CE2 | TYR | A | 417 | 42.728 | 38.056 | 13.274 | 1.00 | 22.53 |
| ATOM | 3162 | CZ | TYR | A | 417 | 42.152 | 37.586 | 12.106 | 1.00 | 23.32 |
| ATOM | 3163 | OH | TYR | A | 417 | 42.133 | 36.225 | 11.888 | 1.00 | 20.94 |
| ATOM | 3164 | N | ILE | A | 418 | 45.014 | 41.940 | 14.176 | 1.00 | 26.21 |
| ATOM | 3165 | CA | ILE | A | 418 | 46.177 | 41.339 | 14.811 | 1.00 | 25.01 |
| ATOM | 3166 | C | ILE | A | 418 | 45.748 | 40.303 | 15.767 | 1.00 | 24.88 |
| ATOM | 3167 | O | ILE | A | 418 | 44.818 | 40.526 | 16.547 | 1.00 | 25.94 |
| ATOM | 3168 | CB | ILE | A | 418 | 47.000 | 42.390 | 15.518 | 1.00 | 25.33 |
| ATOM | 3169 | CG1 | ILE | A | 418 | 47.674 | 43.234 | 14.470 | 1.00 | 28.85 |
| ATOM | 3170 | CG2 | ILE | A | 418 | 48.059 | 41.769 | 16.379 | 1.00 | 24.12 |
| ATOM | 3171 | CD1 | ILE | A | 418 | 48.540 | 44.194 | 15.030 | 1.00 | 31.68 |
| ATOM | 3172 | N | SER | A | 419 | 46.420 | 39.155 | 15.743 | 1.00 | 24.26 |
| ATOM | 3173 | CA | SER | A | 419 | 46.106 | 38.103 | 16.678 | 1.00 | 25.53 |
| ATOM | 3174 | C | SER | A | 419 | 47.312 | 37.280 | 17.061 | 1.00 | 25.14 |
| ATOM | 3175 | O | SER | A | 419 | 48.396 | 37.373 | 16.451 | 1.00 | 25.43 |
| ATOM | 3176 | CB | SER | A | 419 | 45.066 | 37.149 | 16.081 | 1.00 | 26.23 |
| ATOM | 3177 | OG | SER | A | 419 | 45.697 | 36.205 | 15.211 | 1.00 | 28.19 |
| ATOM | 3178 | N | ASN | A | 420 | 47.099 | 36.430 | 18.042 | 1.00 | 25.39 |
| ATOM | 3179 | CA | ASN | A | 420 | 48.130 | 35.491 | 18.489 | 1.00 | 26.17 |
| ATOM | 3180 | C | ASN | A | 420 | 47.898 | 34.036 | 18.027 | 1.00 | 26.94 |
| ATOM | 3181 | O | ASN | A | 420 | 48.406 | 33.088 | 18.641 | 1.00 | 27.30 |
| ATOM | 3182 | CB | ASN | A | 420 | 48.376 | 35.601 | 20.035 | 1.00 | 25.12 |
| ATOM | 3183 | CG | ASN | A | 420 | 47.212 | 35.187 | 20.883 | 1.00 | 24.08 |
| ATOM | 3184 | OD1 | ASN | A | 420 | 47.257 | 35.303 | 22.141 | 1.00 | 25.35 |
| ATOM | 3185 | ND2 | ASN | A | 420 | 46.176 | 34.692 | 20.269 | 1.00 | 19.89 |
| ATOM | 3186 | N | GLU | A | 421 | 47.169 | 33.861 | 16.920 | 1.00 | 28.64 |
| ATOM | 3187 | CA | GLU | A | 421 | 46.899 | 32.514 | 16.372 | 1.00 | 29.10 |
| ATOM | 3188 | C | GLU | A | 421 | 48.151 | 31.761 | 15.959 | 1.00 | 29.52 |
| ATOM | 3189 | O | GLU | A | 421 | 48.269 | 30.590 | 16.247 | 1.00 | 29.85 |
| ATOM | 3190 | CB | GLU | A | 421 | 45.980 | 32.604 | 15.141 | 1.00 | 29.42 |
| ATOM | 3191 | CG | GLU | A | 421 | 45.615 | 31.262 | 14.536 | 1.00 | 29.34 |
| ATOM | 3192 | CD | GLU | A | 421 | 44.558 | 31.362 | 13.432 | 1.00 | 33.30 |
| ATOM | 3193 | OE1 | GLU | A | 421 | 44.398 | 32.432 | 12.827 | 1.00 | 32.10 |
| ATOM | 3194 | OE2 | GLU | A | 421 | 43.872 | 30.354 | 13.176 | 1.00 | 36.08 |
| ATOM | 3195 | N | TYR | A | 422 | 49.081 | 32.435 | 15.287 | 1.00 | 30.59 |
| ATOM | 3196 | CA | TYR | A | 422 | 50.233 | 31.754 | 14.681 | 1.00 | 31.98 |
| ATOM | 3197 | C | TYR | A | 422 | 50.999 | 30.846 | 15.642 | 1.00 | 31.87 |
| ATOM | 3198 | O | TYR | A | 422 | 51.506 | 31.287 | 16.655 | 1.00 | 31.13 |
| ATOM | 3199 | CB | TYR | A | 422 | 51.212 | 32.768 | 14.018 | 1.00 | 32.51 |
| ATOM | 3200 | CG | TYR | A | 422 | 52.199 | 32.109 | 13.069 | 1.00 | 35.45 |
| ATOM | 3201 | CD1 | TYR | A | 422 | 51.754 | 31.303 | 12.014 | 1.00 | 39.38 |
| ATOM | 3202 | CD2 | TYR | A | 422 | 53.568 | 32.294 | 13.200 | 1.00 | 38.76 |
| ATOM | 3203 | CE1 | TYR | A | 422 | 52.649 | 30.698 | 11.126 | 1.00 | 40.25 |
| ATOM | 3204 | CE2 | TYR | A | 422 | 54.476 | 31.674 | 12.306 | 1.00 | 39.86 |
| ATOM | 3205 | CZ | TYR | A | 422 | 53.997 | 30.881 | 11.287 | 1.00 | 39.55 |
| ATOM | 3206 | OH | TYR | A | 422 | 54.860 | 30.275 | 10.416 | 1.00 | 43.28 |
| ATOM | 3207 | N | LYS | A | 423 | 51.042 | 29.566 | 15.305 | 1.00 | 32.75 |
| ATOM | 3208 | CA | LYS | A | 423 | 51.821 | 28.576 | 16.015 | 1.00 | 33.66 |
| ATOM | 3209 | C | LYS | A | 423 | 51.305 | 28.368 | 17.404 | 1.00 | 33.06 |
| ATOM | 3210 | O | LYS | A | 423 | 51.957 | 27.749 | 18.205 | 1.00 | 32.45 |
| ATOM | 3211 | CB | LYS | A | 423 | 53.298 | 28.989 | 16.096 | 1.00 | 34.71 |
| ATOM | 3212 | CG | LYS | A | 423 | 54.060 | 29.012 | 14.786 | 1.00 | 38.92 |
| ATOM | 3213 | CD | LYS | A | 423 | 55.606 | 28.785 | 15.070 | 1.00 | 44.73 |
| ATOM | 3214 | CE | LYS | A | 423 | 56.503 | 28.952 | 13.824 | 1.00 | 47.80 |
| ATOM | 3215 | NZ | LYS | A | 423 | 57.976 | 28.612 | 14.100 | 1.00 | 48.84 |
| ATOM | 3216 | N | GLY | A | 424 | 50.128 | 28.901 | 17.706 | 1.00 | 33.60 |
| ATOM | 3217 | CA | GLY | A | 424 | 49.582 | 28.755 | 19.043 | 1.00 | 32.49 |
| ATOM | 3218 | C | GLY | A | 424 | 50.375 | 29.503 | 20.116 | 1.00 | 32.12 |
| ATOM | 3219 | O | GLY | A | 424 | 50.240 | 29.197 | 21.286 | 1.00 | 31.47 |
| ATOM | 3220 | N | MET | A | 425 | 51.202 | 30.468 | 19.720 | 1.00 | 31.65 |
| ATOM | 3221 | CA | MET | A | 425 | 52.039 | 31.210 | 20.656 | 1.00 | 31.13 |
| ATOM | 3222 | C | MET | A | 425 | 51.326 | 32.484 | 21.169 | 1.00 | 29.99 |
| ATOM | 3223 | O | MET | A | 425 | 51.157 | 33.469 | 20.452 | 1.00 | 28.28 |
| ATOM | 3224 | CB | MET | A | 425 | 53.362 | 31.533 | 19.979 | 1.00 | 32.30 |
| ATOM | 3225 | CG | MET | A | 425 | 54.366 | 30.328 | 19.854 | 1.00 | 34.67 |
| ATOM | 3226 | SD | MET | A | 425 | 55.791 | 30.781 | 18.773 | 1.00 | 43.19 |
| ATOM | 3227 | CE | MET | A | 425 | 56.752 | 31.697 | 19.859 | 1.00 | 40.54 |
| ATOM | 3228 | N | PRO | A | 426 | 50.842 | 32.463 | 22.404 | 1.00 | 29.14 |
| ATOM | 3229 | CA | PRO | A | 426 | 50.045 | 33.599 | 22.894 | 1.00 | 29.32 |
| ATOM | 3230 | C | PRO | A | 426 | 50.830 | 34.917 | 22.991 | 1.00 | 28.68 |
| ATOM | 3231 | O | PRO | A | 426 | 50.261 | 36.028 | 22.904 | 1.00 | 28.13 |
| ATOM | 3232 | CB | PRO | A | 426 | 49.586 | 33.123 | 24.255 | 1.00 | 30.45 |
| ATOM | 3233 | CG | PRO | A | 426 | 49.746 | 31.593 | 24.176 | 1.00 | 30.82 |
| ATOM | 3234 | CD | PRO | A | 426 | 50.957 | 31.390 | 23.398 | 1.00 | 28.81 |
| ATOM | 3235 | N | GLY | A | 427 | 52.137 | 34.794 | 23.123 | 1.00 | 27.82 |
| ATOM | 3236 | CA | GLY | A | 427 | 53.011 | 35.949 | 23.152 | 1.00 | 27.65 |
| ATOM | 3237 | C | GLY | A | 427 | 53.544 | 36.321 | 21.800 | 1.00 | 28.12 |
| ATOM | 3238 | O | GLY | A | 427 | 54.519 | 37.026 | 21.718 | 1.00 | 28.43 |
| ATOM | 3239 | N | GLY | A | 428 | 52.964 | 35.779 | 20.729 | 1.00 | 28.74 |
| ATOM | 3240 | CA | GLY | A | 428 | 53.351 | 36.140 | 19.374 | 1.00 | 27.66 |
| ATOM | 3241 | C | GLY | A | 428 | 52.211 | 36.992 | 18.856 | 1.00 | 27.88 |
| ATOM | 3242 | O | GLY | A | 428 | 51.126 | 36.931 | 19.420 | 1.00 | 27.92 |
| ATOM | 3243 | N | ARG | A | 429 | 52.464 | 37.790 | 17.825 | 1.00 | 28.39 |
| ATOM | 3244 | CA | ARG | A | 429 | 51.474 | 38.656 | 17.193 | 1.00 | 28.66 |
| ATOM | 3245 | C | ARG | A | 429 | 51.675 | 38.800 | 15.676 | 1.00 | 27.70 |
| ATOM | 3246 | O | ARG | A | 429 | 52.789 | 38.921 | 15.211 | 1.00 | 28.68 |
| ATOM | 3247 | CB | ARG | A | 429 | 51.538 | 40.024 | 17.836 | 1.00 | 28.54 |
| ATOM | 3248 | CG | ARG | A | 429 | 51.222 | 39.985 | 19.345 | 1.00 | 33.08 |
| ATOM | 3249 | CD | ARG | A | 429 | 50.733 | 41.296 | 19.829 | 1.00 | 35.58 |
| ATOM | 3250 | NE | ARG | A | 429 | 50.247 | 41.344 | 21.194 | 1.00 | 36.13 |
| ATOM | 3251 | CZ | ARG | A | 429 | 49.937 | 42.510 | 21.785 | 1.00 | 39.81 |
| ATOM | 3252 | NH1 | ARG | A | 429 | 50.070 | 43.661 | 21.095 | 1.00 | 35.49 |
| ATOM | 3253 | NH2 | ARG | A | 429 | 49.505 | 42.545 | 23.058 | 1.00 | 42.19 |
| ATOM | 3254 | N | ASN | A | 430 | 50.571 | 38.825 | 14.919 | 1.00 | 27.66 |
| ATOM | 3255 | CA | ASN | A | 430 | 50.591 | 38.901 | 13.457 | 1.00 | 26.33 |
| ATOM | 3256 | C | ASN | A | 430 | 49.336 | 39.533 | 12.861 | 1.00 | 26.87 |
| ATOM | 3257 | O | ASN | A | 430 | 48.218 | 39.455 | 13.408 | 1.00 | 24.20 |
| ATOM | 3258 | CB | ASN | A | 430 | 50.767 | 37.530 | 12.815 | 1.00 | 26.03 |
| ATOM | 3259 | CG | ASN | A | 430 | 52.193 | 37.015 | 12.895 | 1.00 | 25.47 |
| ATOM | 3260 | OD1 | ASN | A | 430 | 53.094 | 37.458 | 12.154 | 1.00 | 24.95 |
| ATOM | 3261 | ND2 | ASN | A | 430 | 52.410 | 36.087 | 13.799 | 1.00 | 21.72 |
| ATOM | 3262 | N | LEU | A | 431 | 49.531 | 40.139 | 11.697 | 1.00 | 26.92 |
| ATOM | 3263 | CA | LEU | A | 431 | 48.466 | 40.816 | 11.003 | 1.00 | 27.27 |
| ATOM | 3264 | C | LEU | A | 431 | 47.802 | 39.813 | 10.100 | 1.00 | 28.47 |
| ATOM | 3265 | O | LEU | A | 431 | 48.509 | 39.063 | 9.391 | 1.00 | 28.34 |
| ATOM | 3266 | CB | LEU | A | 431 | 49.072 | 41.867 | 10.113 | 1.00 | 27.86 |
| ATOM | 3267 | CG | LEU | A | 431 | 48.429 | 43.221 | 9.893 | 1.00 | 29.78 |
| ATOM | 3268 | CD1 | LEU | A | 431 | 48.707 | 43.681 | 8.457 | 1.00 | 29.33 |
| ATOM | 3269 | CD2 | LEU | A | 431 | 46.966 | 43.267 | 10.216 | 1.00 | 30.92 |
| ATOM | 3270 | N | TYR | A | 432 | 46.464 | 39.842 | 10.074 | 1.00 | 28.64 |
| ATOM | 3271 | CA | TYR | A | 432 | 45.667 | 39.047 | 9.151 | 1.00 | 28.97 |
| ATOM | 3272 | C | TYR | A | 432 | 44.654 | 39.899 | 8.412 | 1.00 | 28.90 |
| ATOM | 3273 | O | TYR | A | 432 | 44.328 | 40.988 | 8.841 | 1.00 | 29.46 |
| ATOM | 3274 | CB | TYR | A | 432 | 44.905 | 37.962 | 9.881 | 1.00 | 29.45 |
| ATOM | 3275 | CG | TYR | A | 432 | 45.762 | 36.984 | 10.591 | 1.00 | 28.94 |
| ATOM | 3276 | CD1 | TYR | A | 432 | 46.443 | 37.348 | 11.729 | 1.00 | 31.85 |
| ATOM | 3277 | CD2 | TYR | A | 432 | 45.861 | 35.686 | 10.158 | 1.00 | 29.80 |
| ATOM | 3278 | CE1 | TYR | A | 432 | 47.242 | 36.460 | 12.407 | 1.00 | 30.13 |
| ATOM | 3279 | CE2 | TYR | A | 432 | 46.635 | 34.774 | 10.830 | 1.00 | 32.21 |
| ATOM | 3280 | CZ | TYR | A | 432 | 47.333 | 35.190 | 11.965 | 1.00 | 30.68 |
| ATOM | 3281 | OH | TYR | A | 432 | 48.103 | 34.332 | 12.656 | 1.00 | 30.32 |
| ATOM | 3282 | N | LYS | A | 433 | 44.128 | 39.334 | 7.325 | 1.00 | 29.50 |
| ATOM | 3283 | CA | LYS | A | 433 | 43.153 | 39.919 | 6.406 | 1.00 | 29.94 |
| ATOM | 3284 | C | LYS | A | 433 | 42.035 | 38.900 | 6.339 | 1.00 | 30.64 |
| ATOM | 3285 | O | LYS | A | 433 | 42.328 | 37.729 | 6.168 | 1.00 | 30.63 |
| ATOM | 3286 | CB | LYS | A | 433 | 43.728 | 39.833 | 4.984 | 1.00 | 30.60 |
| ATOM | 3287 | CG | LYS | A | 433 | 43.650 | 40.975 | 4.056 | 1.00 | 31.91 |
| ATOM | 3288 | CD | LYS | A | 433 | 44.453 | 40.571 | 2.841 | 1.00 | 33.77 |
| ATOM | 3289 | CE | LYS | A | 433 | 44.114 | 41.333 | 1.587 | 1.00 | 36.03 |
| ATOM | 3290 | NZ | LYS | A | 433 | 44.761 | 40.646 | 0.431 | 1.00 | 35.95 |
| ATOM | 3291 | N | ILE | A | 434 | 40.784 | 39.355 | 6.385 | 1.00 | 31.20 |
| ATOM | 3292 | CA | ILE | A | 434 | 39.617 | 38.531 | 6.221 | 1.00 | 31.63 |
| ATOM | 3293 | C | ILE | A | 434 | 38.837 | 39.076 | 5.071 | 1.00 | 31.51 |
| ATOM | 3294 | O | ILE | A | 434 | 38.403 | 40.224 | 5.107 | 1.00 | 31.85 |
| ATOM | 3295 | CB | ILE | A | 434 | 38.675 | 38.672 | 7.412 | 1.00 | 32.38 |
| ATOM | 3296 | CG1 | ILE | A | 434 | 39.288 | 38.134 | 8.684 | 1.00 | 33.28 |
| ATOM | 3297 | CG2 | ILE | A | 434 | 37.413 | 37.945 | 7.099 | 1.00 | 33.44 |
| ATOM | 3298 | CD1 | ILE | A | 434 | 38.482 | 38.485 | 9.933 | 1.00 | 35.68 |
| ATOM | 3299 | N | GLN | A | 435 | 38.580 | 38.254 | 4.078 | 1.00 | 32.13 |
| ATOM | 3300 | CA | GLN | A | 435 | 37.756 | 38.681 | 2.956 | 1.00 | 32.42 |
| ATOM | 3301 | C | GLN | A | 435 | 36.309 | 38.729 | 3.462 | 1.00 | 31.87 |
| ATOM | 3302 | O | GLN | A | 435 | 35.772 | 37.735 | 3.981 | 1.00 | 31.80 |
| ATOM | 3303 | CB | GLN | A | 435 | 37.959 | 37.725 | 1.773 | 1.00 | 33.28 |
| ATOM | 3304 | CG | GLN | A | 435 | 38.283 | 38.438 | 0.474 | 1.00 | 37.80 |
| ATOM | 3305 | CD | GLN | A | 435 | 38.196 | 37.544 | −0.759 | 1.00 | 39.65 |
| ATOM | 3306 | OE1 | GLN | A | 435 | 37.661 | 37.960 | −1.787 | 1.00 | 43.51 |
| ATOM | 3307 | NE2 | GLN | A | 435 | 38.731 | 36.352 | −0.668 | 1.00 | 36.20 |
| ATOM | 3308 | N | LEU | A | 436 | 35.679 | 39.892 | 3.402 | 1.00 | 31.45 |
| ATOM | 3309 | CA | LEU | A | 436 | 34.322 | 40.019 | 3.951 | 1.00 | 32.16 |
| ATOM | 3310 | C | LEU | A | 436 | 33.214 | 39.216 | 3.211 | 1.00 | 33.57 |
| ATOM | 3311 | O | LEU | A | 436 | 32.222 | 38.810 | 3.835 | 1.00 | 32.78 |
| ATOM | 3312 | CB | LEU | A | 436 | 33.967 | 41.505 | 4.095 | 1.00 | 32.40 |
| ATOM | 3313 | CG | LEU | A | 436 | 34.958 | 42.257 | 5.046 | 1.00 | 31.40 |
| ATOM | 3314 | CD1 | LEU | A | 436 | 34.666 | 43.711 | 5.103 | 1.00 | 33.04 |
| ATOM | 3315 | CD2 | LEU | A | 436 | 34.920 | 41.684 | 6.449 | 1.00 | 30.51 |
| ATOM | 3316 | N | SER | A | 437 | 33.418 | 38.892 | 1.938 | 1.00 | 34.25 |
| ATOM | 3317 | CA | SER | A | 437 | 32.403 | 38.123 | 1.202 | 1.00 | 36.72 |
| ATOM | 3318 | C | SER | A | 437 | 32.508 | 36.603 | 1.433 | 1.00 | 36.62 |
| ATOM | 3319 | O | SER | A | 437 | 31.851 | 35.815 | 0.739 | 1.00 | 40.69 |
| ATOM | 3320 | CB | SER | A | 437 | 32.516 | 38.418 | −0.282 | 1.00 | 36.83 |
| ATOM | 3321 | OG | SER | A | 437 | 33.806 | 38.070 | −0.709 | 1.00 | 38.78 |
| ATOM | 3322 | N | ASP | A | 438 | 33.393 | 36.215 | 2.363 | 1.00 | 35.66 |
| ATOM | 3323 | CA | ASP | A | 438 | 33.577 | 34.814 | 2.806 | 1.00 | 34.84 |
| ATOM | 3324 | C | ASP | A | 438 | 34.562 | 34.703 | 3.979 | 1.00 | 33.83 |
| ATOM | 3325 | O | ASP | A | 438 | 35.765 | 34.547 | 3.790 | 1.00 | 33.54 |
| ATOM | 3326 | CB | ASP | A | 438 | 34.101 | 33.920 | 1.684 | 1.00 | 34.29 |
| ATOM | 3327 | CG | ASP | A | 438 | 34.300 | 32.480 | 2.139 | 1.00 | 33.79 |
| ATOM | 3328 | OD1 | ASP | A | 438 | 34.227 | 32.222 | 3.362 | 1.00 | 31.81 |
| ATOM | 3329 | OD2 | ASP | A | 438 | 34.513 | 31.535 | 1.354 | 1.00 | 32.29 |
| ATOM | 3330 | N | TYR | A | 439 | 34.037 | 34.660 | 5.183 | 1.00 | 33.43 |
| ATOM | 3331 | CA | TYR | A | 439 | 34.867 | 34.735 | 6.372 | 1.00 | 33.14 |
| ATOM | 3332 | C | TYR | A | 439 | 35.881 | 33.611 | 6.504 | 1.00 | 33.75 |
| ATOM | 3333 | O | TYR | A | 439 | 36.804 | 33.712 | 7.322 | 1.00 | 32.43 |
| ATOM | 3334 | CB | TYR | A | 439 | 34.009 | 34.815 | 7.618 | 1.00 | 32.78 |
| ATOM | 3335 | CG | TYR | A | 439 | 33.032 | 35.953 | 7.614 | 1.00 | 31.50 |
| ATOM | 3336 | CD1 | TYR | A | 439 | 33.363 | 37.196 | 7.084 | 1.00 | 29.56 |
| ATOM | 3337 | CD2 | TYR | A | 439 | 31.763 | 35.773 | 8.127 | 1.00 | 31.06 |
| ATOM | 3338 | CE1 | TYR | A | 439 | 32.453 | 38.222 | 7.081 | 1.00 | 32.55 |
| ATOM | 3339 | CE2 | TYR | A | 439 | 30.846 | 36.776 | 8.107 | 1.00 | 32.77 |
| ATOM | 3340 | CZ | TYR | A | 439 | 31.177 | 37.994 | 7.622 | 1.00 | 32.72 |
| ATOM | 3341 | OH | TYR | A | 439 | 30.215 | 38.969 | 7.671 | 1.00 | 34.45 |
| ATOM | 3342 | N | THR | A | 440 | 35.743 | 32.555 | 5.705 | 1.00 | 33.09 |
| ATOM | 3343 | CA | THR | A | 440 | 36.749 | 31.515 | 5.737 | 1.00 | 33.93 |
| ATOM | 3344 | C | THR | A | 440 | 38.011 | 31.959 | 5.002 | 1.00 | 33.99 |
| ATOM | 3345 | O | THR | A | 440 | 39.049 | 31.357 | 5.164 | 1.00 | 34.35 |
| ATOM | 3346 | CB | THR | A | 440 | 36.222 | 30.188 | 5.140 | 1.00 | 34.79 |
| ATOM | 3347 | OG1 | THR | A | 440 | 35.854 | 30.386 | 3.773 | 1.00 | 35.49 |
| ATOM | 3348 | CG2 | THR | A | 440 | 34.914 | 29.733 | 5.808 | 1.00 | 35.92 |
| ATOM | 3349 | N | LYS | A | 441 | 37.949 | 32.994 | 4.167 | 1.00 | 34.58 |
| ATOM | 3350 | CA | LYS | A | 441 | 39.161 | 33.419 | 3.485 | 1.00 | 34.94 |
| ATOM | 3351 | C | LYS | A | 441 | 39.982 | 34.419 | 4.316 | 1.00 | 34.57 |
| ATOM | 3352 | O | LYS | A | 441 | 39.780 | 35.645 | 4.218 | 1.00 | 33.38 |
| ATOM | 3353 | CB | LYS | A | 441 | 38.843 | 33.969 | 2.099 | 1.00 | 36.12 |
| ATOM | 3354 | CG | LYS | A | 441 | 38.246 | 32.904 | 1.199 | 1.00 | 38.45 |
| ATOM | 3355 | CD | LYS | A | 441 | 37.943 | 33.414 | −0.196 | 1.00 | 42.38 |
| ATOM | 3356 | CE | LYS | A | 441 | 37.790 | 32.241 | −1.204 | 1.00 | 44.53 |
| ATOM | 3357 | NZ | LYS | A | 441 | 37.079 | 32.642 | −2.444 | 1.00 | 44.38 |
| ATOM | 3358 | N | VAL | A | 442 | 40.918 | 33.844 | 5.081 | 1.00 | 34.16 |
| ATOM | 3359 | CA | VAL | A | 442 | 41.807 | 34.527 | 6.017 | 1.00 | 34.73 |
| ATOM | 3360 | C | VAL | A | 442 | 43.264 | 34.386 | 5.623 | 1.00 | 34.50 |
| ATOM | 3361 | O | VAL | A | 442 | 43.788 | 33.300 | 5.575 | 1.00 | 34.09 |
| ATOM | 3362 | CB | VAL | A | 442 | 41.744 | 33.883 | 7.405 | 1.00 | 34.66 |
| ATOM | 3363 | CG1 | VAL | A | 442 | 42.371 | 34.805 | 8.445 | 1.00 | 36.89 |
| ATOM | 3364 | CG2 | VAL | A | 442 | 40.351 | 33.605 | 7.809 | 1.00 | 35.16 |
| ATOM | 3365 | N | THR | A | 443 | 43.922 | 35.486 | 5.338 | 1.00 | 34.30 |
| ATOM | 3366 | CA | THR | A | 443 | 45.312 | 35.425 | 4.983 | 1.00 | 34.85 |
| ATOM | 3367 | C | THR | A | 443 | 46.177 | 35.999 | 6.132 | 1.00 | 34.39 |
| ATOM | 3368 | O | THR | A | 443 | 45.870 | 37.053 | 6.659 | 1.00 | 33.87 |
| ATOM | 3369 | CB | THR | A | 443 | 45.543 | 36.278 | 3.734 | 1.00 | 34.69 |
| ATOM | 3370 | OG1 | THR | A | 443 | 44.628 | 35.922 | 2.700 | 1.00 | 36.35 |
| ATOM | 3371 | CG2 | THR | A | 443 | 46.839 | 35.955 | 3.151 | 1.00 | 37.11 |
| ATOM | 3372 | N | CYS | A | 444 | 47.238 | 35.314 | 6.515 | 1.00 | 33.85 |
| ATOM | 3373 | CA | CYS | A | 444 | 48.191 | 35.920 | 7.424 | 1.00 | 33.80 |
| ATOM | 3374 | C | CYS | A | 444 | 49.197 | 36.741 | 6.612 | 1.00 | 32.96 |
| ATOM | 3375 | O | CYS | A | 444 | 49.896 | 36.202 | 5.763 | 1.00 | 33.20 |
| ATOM | 3376 | CB | CYS | A | 444 | 48.932 | 34.893 | 8.256 | 1.00 | 34.01 |
| ATOM | 3377 | SG | CYS | A | 444 | 49.930 | 35.769 | 9.498 | 1.00 | 35.63 |
| ATOM | 3378 | N | LEU | A | 445 | 49.259 | 38.049 | 6.846 | 1.00 | 31.35 |
| ATOM | 3379 | CA | LEU | A | 445 | 50.115 | 38.930 | 6.035 | 1.00 | 30.34 |
| ATOM | 3380 | C | LEU | A | 445 | 51.540 | 39.101 | 6.506 | 1.00 | 29.68 |
| ATOM | 3381 | O | LEU | A | 445 | 52.381 | 39.600 | 5.755 | 1.00 | 29.03 |
| ATOM | 3382 | CB | LEU | A | 445 | 49.511 | 40.320 | 5.989 | 1.00 | 30.44 |
| ATOM | 3383 | CG | LEU | A | 445 | 48.082 | 40.371 | 5.441 | 1.00 | 31.52 |
| ATOM | 3384 | CD1 | LEU | A | 445 | 47.519 | 41.766 | 5.595 | 1.00 | 31.49 |
| ATOM | 3385 | CD2 | LEU | A | 445 | 48.031 | 39.979 | 3.970 | 1.00 | 30.01 |
| ATOM | 3386 | N | SER | A | 446 | 51.809 | 38.716 | 7.751 | 1.00 | 28.93 |
| ATOM | 3387 | CA | SER | A | 446 | 53.115 | 38.914 | 8.335 | 1.00 | 28.62 |
| ATOM | 3388 | C | SER | A | 446 | 53.814 | 37.628 | 8.738 | 1.00 | 29.11 |
| ATOM | 3389 | O | SER | A | 446 | 55.032 | 37.622 | 8.909 | 1.00 | 29.02 |
| ATOM | 3390 | CB | SER | A | 446 | 53.001 | 39.829 | 9.553 | 1.00 | 27.81 |
| ATOM | 3391 | OG | SER | A | 446 | 52.252 | 39.228 | 10.616 | 1.00 | 24.74 |
| ATOM | 3392 | N | CYS | A | 447 | 53.042 | 36.569 | 8.909 | 1.00 | 29.85 |
| ATOM | 3393 | CA | CYS | A | 447 | 53.536 | 35.330 | 9.492 | 1.00 | 32.67 |
| ATOM | 3394 | C | CYS | A | 447 | 54.827 | 34.834 | 8.884 | 1.00 | 33.54 |
| ATOM | 3395 | O | CYS | A | 447 | 55.682 | 34.357 | 9.614 | 1.00 | 33.36 |
| ATOM | 3396 | CB | CYS | A | 447 | 52.484 | 34.203 | 9.376 | 1.00 | 33.28 |
| ATOM | 3397 | SG | CYS | A | 447 | 51.032 | 34.431 | 10.472 | 1.00 | 40.47 |
| ATOM | 3398 | N | GLU | A | 448 | 54.940 | 34.924 | 7.556 | 1.00 | 34.86 |
| ATOM | 3399 | CA | GLU | A | 448 | 56.066 | 34.346 | 6.817 | 1.00 | 36.63 |
| ATOM | 3400 | C | GLU | A | 448 | 57.017 | 35.375 | 6.270 | 1.00 | 36.08 |
| ATOM | 3401 | O | GLU | A | 448 | 57.845 | 35.036 | 5.447 | 1.00 | 35.92 |
| ATOM | 3402 | CB | GLU | A | 448 | 55.592 | 33.541 | 5.587 | 1.00 | 37.55 |
| ATOM | 3403 | CG | GLU | A | 448 | 54.507 | 32.502 | 5.845 | 1.00 | 42.12 |
| ATOM | 3404 | CD | GLU | A | 448 | 55.086 | 31.151 | 6.204 | 1.00 | 48.74 |
| ATOM | 3405 | OE1 | GLU | A | 448 | 55.945 | 31.107 | 7.129 | 1.00 | 50.57 |
| ATOM | 3406 | OE2 | GLU | A | 448 | 54.703 | 30.139 | 5.534 | 1.00 | 52.73 |
| ATOM | 3407 | N | LEU | A | 449 | 56.898 | 36.629 | 6.673 | 1.00 | 34.96 |
| ATOM | 3408 | CA | LEU | A | 449 | 57.825 | 37.606 | 6.149 | 1.00 | 34.44 |
| ATOM | 3409 | C | LEU | A | 449 | 59.294 | 37.265 | 6.502 | 1.00 | 34.44 |
| ATOM | 3410 | O | LEU | A | 449 | 60.147 | 37.251 | 5.621 | 1.00 | 33.97 |
| ATOM | 3411 | CB | LEU | A | 449 | 57.429 | 39.023 | 6.552 | 1.00 | 33.23 |
| ATOM | 3412 | CG | LEU | A | 449 | 56.115 | 39.457 | 5.858 | 1.00 | 35.54 |
| ATOM | 3413 | CD1 | LEU | A | 449 | 55.677 | 40.857 | 6.339 | 1.00 | 35.36 |
| ATOM | 3414 | CD2 | LEU | A | 449 | 56.138 | 39.422 | 4.295 | 1.00 | 34.45 |
| ATOM | 3415 | N | ASN | A | 450 | 59.586 | 37.025 | 7.775 | 1.00 | 34.23 |
| ATOM | 3416 | CA | ASN | A | 450 | 60.928 | 36.689 | 8.241 | 1.00 | 34.36 |
| ATOM | 3417 | C | ASN | A | 450 | 60.603 | 35.979 | 9.541 | 1.00 | 33.70 |
| ATOM | 3418 | O | ASN | A | 450 | 60.626 | 36.601 | 10.586 | 1.00 | 31.73 |
| ATOM | 3419 | CB | ASN | A | 450 | 61.811 | 37.938 | 8.517 | 1.00 | 35.46 |
| ATOM | 3420 | CG | ASN | A | 450 | 61.785 | 39.000 | 7.379 | 1.00 | 39.31 |
| ATOM | 3421 | OD1 | ASN | A | 450 | 62.830 | 39.335 | 6.764 | 1.00 | 42.36 |
| ATOM | 3422 | ND2 | ASN | A | 450 | 60.612 | 39.545 | 7.120 | 1.00 | 39.73 |
| ATOM | 3423 | N | PRO | A | 451 | 60.305 | 34.684 | 9.480 | 1.00 | 34.50 |
| ATOM | 3424 | CA | PRO | A | 451 | 59.725 | 33.963 | 10.625 | 1.00 | 35.42 |
| ATOM | 3425 | C | PRO | A | 451 | 60.614 | 33.694 | 11.792 | 1.00 | 35.46 |
| ATOM | 3426 | O | PRO | A | 451 | 60.088 | 33.430 | 12.863 | 1.00 | 35.48 |
| ATOM | 3427 | CB | PRO | A | 451 | 59.318 | 32.608 | 10.041 | 1.00 | 35.72 |
| ATOM | 3428 | CG | PRO | A | 451 | 59.652 | 32.646 | 8.570 | 1.00 | 34.74 |
| ATOM | 3429 | CD | PRO | A | 451 | 60.536 | 33.793 | 8.322 | 1.00 | 35.07 |
| ATOM | 3430 | N | GLU | A | 452 | 61.918 | 33.728 | 11.606 | 1.00 | 36.10 |
| ATOM | 3431 | CA | GLU | A | 452 | 62.809 | 33.444 | 12.697 | 1.00 | 37.19 |
| ATOM | 3432 | C | GLU | A | 452 | 63.138 | 34.796 | 13.333 | 1.00 | 35.91 |
| ATOM | 3433 | O | GLU | A | 452 | 63.356 | 34.883 | 14.500 | 1.00 | 36.70 |
| ATOM | 3434 | CB | GLU | A | 452 | 64.066 | 32.697 | 12.202 | 1.00 | 38.95 |
| ATOM | 3435 | CG | GLU | A | 452 | 63.927 | 31.164 | 12.122 | 1.00 | 44.32 |
| ATOM | 3436 | CD | GLU | A | 452 | 63.457 | 30.631 | 10.758 | 1.00 | 51.15 |
| ATOM | 3437 | OE1 | GLU | A | 452 | 62.294 | 30.923 | 10.354 | 1.00 | 53.26 |
| ATOM | 3438 | OE2 | GLU | A | 452 | 64.256 | 29.892 | 10.089 | 1.00 | 56.31 |
| ATOM | 3439 | N | ARG | A | 453 | 63.120 | 35.878 | 12.579 | 1.00 | 34.02 |
| ATOM | 3440 | CA | ARG | A | 453 | 63.451 | 37.157 | 13.189 | 1.00 | 32.24 |
| ATOM | 3441 | C | ARG | A | 453 | 62.219 | 37.953 | 13.712 | 1.00 | 31.58 |
| ATOM | 3442 | O | ARG | A | 453 | 62.326 | 38.802 | 14.597 | 1.00 | 30.26 |
| ATOM | 3443 | CB | ARG | A | 453 | 64.186 | 38.017 | 12.182 | 1.00 | 31.61 |
| ATOM | 3444 | CG | ARG | A | 453 | 64.295 | 39.448 | 12.600 | 1.00 | 32.11 |
| ATOM | 3445 | CD | ARG | A | 453 | 65.075 | 40.301 | 11.626 | 1.00 | 33.78 |
| ATOM | 3446 | NE | ARG | A | 453 | 65.181 | 41.697 | 12.055 | 1.00 | 32.76 |
| ATOM | 3447 | CZ | ARG | A | 453 | 65.862 | 42.602 | 11.380 | 1.00 | 32.63 |
| ATOM | 3448 | NH1 | ARG | A | 453 | 66.501 | 42.213 | 10.296 | 1.00 | 29.81 |
| ATOM | 3449 | NH2 | ARG | A | 453 | 65.951 | 43.881 | 11.793 | 1.00 | 31.21 |
| ATOM | 3450 | N | CYS | A | 454 | 61.061 | 37.664 | 13.147 | 1.00 | 30.42 |
| ATOM | 3451 | CA | CYS | A | 454 | 59.876 | 38.453 | 13.396 | 1.00 | 29.70 |
| ATOM | 3452 | C | CYS | A | 454 | 58.670 | 37.649 | 13.738 | 1.00 | 29.35 |
| ATOM | 3453 | O | CYS | A | 454 | 58.098 | 37.039 | 12.867 | 1.00 | 30.20 |
| ATOM | 3454 | CB | CYS | A | 454 | 59.579 | 39.262 | 12.163 | 1.00 | 29.27 |
| ATOM | 3455 | SG | CYS | A | 454 | 60.790 | 40.536 | 11.951 | 1.00 | 29.63 |
| ATOM | 3456 | N | GLN | A | 455 | 58.269 | 37.693 | 15.005 | 1.00 | 29.22 |
| ATOM | 3457 | CA | GLN | A | 455 | 57.087 | 36.993 | 15.516 | 1.00 | 29.05 |
| ATOM | 3458 | C | GLN | A | 455 | 56.162 | 37.906 | 16.337 | 1.00 | 28.56 |
| ATOM | 3459 | O | GLN | A | 455 | 55.245 | 37.423 | 16.997 | 1.00 | 29.64 |
| ATOM | 3460 | CB | GLN | A | 455 | 57.493 | 35.796 | 16.368 | 1.00 | 28.83 |
| ATOM | 3461 | CG | GLN | A | 455 | 58.178 | 34.676 | 15.550 | 1.00 | 30.86 |
| ATOM | 3462 | CD | GLN | A | 455 | 59.028 | 33.712 | 16.408 | 1.00 | 33.92 |
| ATOM | 3463 | OE1 | GLN | A | 455 | 58.881 | 33.616 | 17.639 | 1.00 | 36.61 |
| ATOM | 3464 | NE2 | GLN | A | 455 | 59.909 | 33.011 | 15.756 | 1.00 | 38.34 |
| ATOM | 3465 | N | TYR | A | 456 | 56.381 | 39.216 | 16.289 | 1.00 | 27.49 |
| ATOM | 3466 | CA | TYR | A | 456 | 55.570 | 40.163 | 17.053 | 1.00 | 26.38 |
| ATOM | 3467 | C | TYR | A | 456 | 55.436 | 41.437 | 16.272 | 1.00 | 25.33 |
| ATOM | 3468 | O | TYR | A | 456 | 56.342 | 42.254 | 16.278 | 1.00 | 26.26 |
| ATOM | 3469 | CB | TYR | A | 456 | 56.265 | 40.497 | 18.391 | 1.00 | 26.47 |
| ATOM | 3470 | CG | TYR | A | 456 | 55.357 | 41.032 | 19.483 | 1.00 | 23.54 |
| ATOM | 3471 | CD1 | TYR | A | 456 | 54.969 | 42.361 | 19.514 | 1.00 | 22.01 |
| ATOM | 3472 | CD2 | TYR | A | 456 | 54.938 | 40.205 | 20.510 | 1.00 | 22.91 |
| ATOM | 3473 | CE1 | TYR | A | 456 | 54.181 | 42.876 | 20.588 | 1.00 | 23.76 |
| ATOM | 3474 | CE2 | TYR | A | 456 | 54.126 | 40.684 | 21.551 | 1.00 | 24.99 |
| ATOM | 3475 | CZ | TYR | A | 456 | 53.755 | 42.019 | 21.581 | 1.00 | 24.52 |
| ATOM | 3476 | OH | TYR | A | 456 | 52.958 | 42.443 | 22.623 | 1.00 | 32.80 |
| ATOM | 3477 | N | TYR | A | 457 | 54.298 | 41.627 | 15.643 | 1.00 | 24.21 |
| ATOM | 3478 | CA | TYR | A | 457 | 54.063 | 42.767 | 14.773 | 1.00 | 23.89 |
| ATOM | 3479 | C | TYR | A | 457 | 52.990 | 43.731 | 15.313 | 1.00 | 24.27 |
| ATOM | 3480 | O | TYR | A | 457 | 52.067 | 43.282 | 16.028 | 1.00 | 23.48 |
| ATOM | 3481 | CB | TYR | A | 457 | 53.524 | 42.197 | 13.454 | 1.00 | 24.73 |
| ATOM | 3482 | CG | TYR | A | 457 | 54.585 | 41.559 | 12.548 | 1.00 | 25.26 |
| ATOM | 3483 | CD1 | TYR | A | 457 | 55.297 | 42.334 | 11.675 | 1.00 | 25.50 |
| ATOM | 3484 | CD2 | TYR | A | 457 | 54.812 | 40.204 | 12.549 | 1.00 | 25.73 |
| ATOM | 3485 | CE1 | TYR | A | 457 | 56.237 | 41.807 | 10.827 | 1.00 | 29.31 |
| ATOM | 3486 | CE2 | TYR | A | 457 | 55.769 | 39.642 | 11.701 | 1.00 | 26.68 |
| ATOM | 3487 | CZ | TYR | A | 457 | 56.470 | 40.468 | 10.835 | 1.00 | 26.22 |
| ATOM | 3488 | OH | TYR | A | 457 | 57.408 | 40.006 | 9.979 | 1.00 | 28.29 |
| ATOM | 3489 | N | SER | A | 458 | 53.151 | 45.028 | 15.011 | 1.00 | 23.41 |
| ATOM | 3490 | CA | SER | A | 458 | 52.075 | 45.987 | 15.118 | 1.00 | 24.13 |
| ATOM | 3491 | C | SER | A | 458 | 52.007 | 46.589 | 13.738 | 1.00 | 23.05 |
| ATOM | 3492 | O | SER | A | 458 | 52.877 | 46.344 | 12.906 | 1.00 | 21.75 |
| ATOM | 3493 | CB | SER | A | 458 | 52.258 | 47.064 | 16.189 | 1.00 | 23.41 |
| ATOM | 3494 | OG | SER | A | 458 | 53.293 | 47.909 | 15.806 | 1.00 | 26.48 |
| ATOM | 3495 | N | VAL | A | 459 | 50.981 | 47.385 | 13.484 | 1.00 | 23.05 |
| ATOM | 3496 | CA | VAL | A | 459 | 50.780 | 47.908 | 12.144 | 1.00 | 22.91 |
| ATOM | 3497 | C | VAL | A | 459 | 50.197 | 49.285 | 12.149 | 1.00 | 23.22 |
| ATOM | 3498 | O | VAL | A | 459 | 49.449 | 49.613 | 13.051 | 1.00 | 20.96 |
| ATOM | 3499 | CB | VAL | A | 459 | 49.782 | 47.064 | 11.372 | 1.00 | 23.35 |
| ATOM | 3500 | CG1 | VAL | A | 459 | 48.322 | 47.173 | 11.992 | 1.00 | 20.67 |
| ATOM | 3501 | CG2 | VAL | A | 459 | 49.776 | 47.518 | 9.941 | 1.00 | 23.93 |
| ATOM | 3502 | N | SER | A | 460 | 50.554 | 50.081 | 11.132 | 1.00 | 23.90 |
| ATOM | 3503 | CA | SER | A | 460 | 50.002 | 51.411 | 10.922 | 1.00 | 24.93 |
| ATOM | 3504 | C | SER | A | 460 | 49.569 | 51.602 | 9.488 | 1.00 | 25.93 |
| ATOM | 3505 | O | SER | A | 460 | 50.391 | 51.569 | 8.550 | 1.00 | 22.55 |
| ATOM | 3506 | CB | SER | A | 460 | 50.985 | 52.509 | 11.301 | 1.00 | 25.72 |
| ATOM | 3507 | OG | SER | A | 460 | 50.539 | 53.740 | 10.742 | 1.00 | 27.67 |
| ATOM | 3508 | N | PHE | A | 461 | 48.252 | 51.754 | 9.329 | 1.00 | 26.78 |
| ATOM | 3509 | CA | PHE | A | 461 | 47.633 | 51.920 | 8.026 | 1.00 | 27.31 |
| ATOM | 3510 | C | PHE | A | 461 | 47.418 | 53.377 | 7.628 | 1.00 | 28.08 |
| ATOM | 3511 | O | PHE | A | 461 | 47.054 | 54.190 | 8.443 | 1.00 | 28.52 |
| ATOM | 3512 | CB | PHE | A | 461 | 46.273 | 51.252 | 8.034 | 1.00 | 26.92 |
| ATOM | 3513 | CG | PHE | A | 461 | 46.299 | 49.777 | 7.822 | 1.00 | 25.25 |
| ATOM | 3514 | CD1 | PHE | A | 461 | 46.201 | 49.241 | 6.552 | 1.00 | 27.14 |
| ATOM | 3515 | CD2 | PHE | A | 461 | 46.327 | 48.920 | 8.894 | 1.00 | 27.63 |
| ATOM | 3516 | CE1 | PHE | A | 461 | 46.191 | 47.888 | 6.350 | 1.00 | 25.35 |
| ATOM | 3517 | CE2 | PHE | A | 461 | 46.329 | 47.570 | 8.713 | 1.00 | 25.76 |
| ATOM | 3518 | CZ | PHE | A | 461 | 46.271 | 47.045 | 7.447 | 1.00 | 28.77 |
| ATOM | 3519 | N | SER | A | 462 | 47.631 | 53.691 | 6.347 | 1.00 | 29.97 |
| ATOM | 3520 | CA | SER | A | 462 | 47.246 | 54.988 | 5.778 | 1.00 | 30.27 |
| ATOM | 3521 | C | SER | A | 462 | 45.723 | 55.127 | 5.749 | 1.00 | 31.48 |
| ATOM | 3522 | O | SER | A | 462 | 44.993 | 54.176 | 5.972 | 1.00 | 30.72 |
| ATOM | 3523 | CB | SER | A | 462 | 47.737 | 55.097 | 4.329 | 1.00 | 30.83 |
| ATOM | 3524 | OG | SER | A | 462 | 46.950 | 54.280 | 3.490 | 1.00 | 28.63 |
| ATOM | 3525 | N | LYS | A | 463 | 45.240 | 56.313 | 5.429 | 1.00 | 33.73 |
| ATOM | 3526 | CA | LYS | A | 463 | 43.799 | 56.526 | 5.293 | 1.00 | 35.96 |
| ATOM | 3527 | C | LYS | A | 463 | 43.305 | 55.681 | 4.120 | 1.00 | 36.37 |
| ATOM | 3528 | O | LYS | A | 463 | 44.018 | 55.519 | 3.105 | 1.00 | 38.14 |
| ATOM | 3529 | CB | LYS | A | 463 | 43.492 | 58.014 | 5.103 | 1.00 | 36.63 |
| ATOM | 3530 | CG | LYS | A | 463 | 44.403 | 58.902 | 5.944 | 1.00 | 39.71 |
| ATOM | 3531 | CD | LYS | A | 463 | 43.822 | 60.236 | 6.432 | 1.00 | 45.02 |
| ATOM | 3532 | CE | LYS | A | 463 | 44.530 | 60.660 | 7.780 | 1.00 | 48.04 |
| ATOM | 3533 | NZ | LYS | A | 463 | 44.959 | 62.107 | 7.840 | 1.00 | 48.63 |
| ATOM | 3534 | N | GLU | A | 464 | 42.132 | 55.080 | 4.269 | 1.00 | 35.63 |
| ATOM | 3535 | CA | GLU | A | 464 | 41.549 | 54.257 | 3.209 | 1.00 | 35.32 |
| ATOM | 3536 | C | GLU | A | 464 | 42.350 | 52.989 | 3.114 | 1.00 | 33.42 |
| ATOM | 3537 | O | GLU | A | 464 | 42.107 | 52.137 | 2.259 | 1.00 | 32.17 |
| ATOM | 3538 | CB | GLU | A | 464 | 41.473 | 54.978 | 1.849 | 1.00 | 36.66 |
| ATOM | 3539 | CG | GLU | A | 464 | 40.178 | 55.783 | 1.625 | 1.00 | 41.82 |
| ATOM | 3540 | CD | GLU | A | 464 | 38.919 | 54.943 | 1.348 | 1.00 | 49.22 |
| ATOM | 3541 | OE1 | GLU | A | 464 | 38.556 | 54.770 | 0.164 | 1.00 | 50.56 |
| ATOM | 3542 | OE2 | GLU | A | 464 | 38.259 | 54.483 | 2.310 | 1.00 | 53.57 |
| ATOM | 3543 | N | ALA | A | 465 | 43.302 | 52.871 | 4.031 | 1.00 | 32.42 |
| ATOM | 3544 | CA | ALA | A | 465 | 44.115 | 51.683 | 4.130 | 1.00 | 31.60 |
| ATOM | 3545 | C | ALA | A | 465 | 44.746 | 51.223 | 2.805 | 1.00 | 30.62 |
| ATOM | 3546 | O | ALA | A | 465 | 44.907 | 50.035 | 2.563 | 1.00 | 27.72 |
| ATOM | 3547 | CB | ALA | A | 465 | 43.279 | 50.550 | 4.737 | 1.00 | 32.19 |
| ATOM | 3548 | N | LYS | A | 466 | 45.108 | 52.139 | 1.934 | 1.00 | 31.42 |
| ATOM | 3549 | CA | LYS | A | 466 | 45.748 | 51.643 | 0.743 | 1.00 | 32.12 |
| ATOM | 3550 | C | LYS | A | 466 | 47.192 | 51.168 | 1.038 | 1.00 | 31.32 |
| ATOM | 3551 | O | LYS | A | 466 | 47.687 | 50.345 | 0.299 | 1.00 | 30.10 |
| ATOM | 3552 | CB | LYS | A | 466 | 45.656 | 52.630 | −0.428 | 1.00 | 33.58 |
| ATOM | 3553 | CG | LYS | A | 466 | 44.196 | 52.978 | −0.869 | 1.00 | 38.27 |
| ATOM | 3554 | CD | LYS | A | 466 | 43.427 | 51.805 | −1.556 | 1.00 | 43.92 |
| ATOM | 3555 | CE | LYS | A | 466 | 41.904 | 52.062 | −1.554 | 1.00 | 47.17 |
| ATOM | 3556 | NZ | LYS | A | 466 | 41.126 | 51.288 | −2.589 | 1.00 | 48.39 |
| ATOM | 3557 | N | TYR | A | 467 | 47.827 | 51.635 | 2.127 | 1.00 | 31.47 |
| ATOM | 3558 | CA | TYR | A | 467 | 49.198 | 51.263 | 2.498 | 1.00 | 30.89 |
| ATOM | 3559 | C | TYR | A | 467 | 49.333 | 50.950 | 4.001 | 1.00 | 30.71 |
| ATOM | 3560 | O | TYR | A | 467 | 48.572 | 51.497 | 4.829 | 1.00 | 30.78 |
| ATOM | 3561 | CB | TYR | A | 467 | 50.099 | 52.423 | 2.122 | 1.00 | 31.64 |
| ATOM | 3562 | CG | TYR | A | 467 | 50.056 | 52.721 | 0.646 | 1.00 | 31.24 |
| ATOM | 3563 | CD1 | TYR | A | 467 | 50.704 | 51.896 | −0.260 | 1.00 | 32.67 |
| ATOM | 3564 | CD2 | TYR | A | 467 | 49.383 | 53.822 | 0.159 | 1.00 | 30.99 |
| ATOM | 3565 | CE1 | TYR | A | 467 | 50.672 | 52.162 | −1.614 | 1.00 | 34.60 |
| ATOM | 3566 | CE2 | TYR | A | 467 | 49.333 | 54.093 | −1.184 | 1.00 | 31.48 |
| ATOM | 3567 | CZ | TYR | A | 467 | 49.976 | 53.257 | −2.072 | 1.00 | 34.16 |
| ATOM | 3568 | OH | TYR | A | 467 | 49.927 | 53.534 | −3.412 | 1.00 | 34.89 |
| ATOM | 3569 | N | TYR | A | 468 | 50.252 | 50.044 | 4.343 | 1.00 | 29.12 |
| ATOM | 3570 | CA | TYR | A | 468 | 50.549 | 49.763 | 5.731 | 1.00 | 28.87 |
| ATOM | 3571 | C | TYR | A | 468 | 52.062 | 49.651 | 6.052 | 1.00 | 28.68 |
| ATOM | 3572 | O | TYR | A | 468 | 52.877 | 49.243 | 5.210 | 1.00 | 28.35 |
| ATOM | 3573 | CB | TYR | A | 468 | 49.772 | 48.537 | 6.288 | 1.00 | 29.07 |
| ATOM | 3574 | CG | TYR | A | 468 | 49.899 | 47.194 | 5.553 | 1.00 | 28.06 |
| ATOM | 3575 | CD1 | TYR | A | 468 | 49.122 | 46.912 | 4.438 | 1.00 | 28.62 |
| ATOM | 3576 | CD2 | TYR | A | 468 | 50.698 | 46.193 | 6.045 | 1.00 | 26.47 |
| ATOM | 3577 | CE1 | TYR | A | 468 | 49.194 | 45.691 | 3.813 | 1.00 | 28.32 |
| ATOM | 3578 | CE2 | TYR | A | 468 | 50.782 | 44.984 | 5.446 | 1.00 | 26.79 |
| ATOM | 3579 | CZ | TYR | A | 468 | 50.033 | 44.737 | 4.311 | 1.00 | 28.16 |
| ATOM | 3580 | OH | TYR | A | 468 | 50.124 | 43.527 | 3.688 | 1.00 | 29.41 |
| ATOM | 3581 | N | GLN | A | 469 | 52.412 | 50.086 | 7.255 | 1.00 | 27.13 |
| ATOM | 3582 | CA | GLN | A | 469 | 53.763 | 49.962 | 7.777 | 1.00 | 27.19 |
| ATOM | 3583 | C | GLN | A | 469 | 53.697 | 48.856 | 8.804 | 1.00 | 27.67 |
| ATOM | 3584 | O | GLN | A | 469 | 52.864 | 48.893 | 9.715 | 1.00 | 26.15 |
| ATOM | 3585 | CB | GLN | A | 469 | 54.221 | 51.271 | 8.435 | 1.00 | 27.15 |
| ATOM | 3586 | CG | GLN | A | 469 | 55.515 | 51.121 | 9.271 | 1.00 | 27.66 |
| ATOM | 3587 | CD | GLN | A | 469 | 55.813 | 52.307 | 10.219 | 1.00 | 29.76 |
| ATOM | 3588 | OE1 | GLN | A | 469 | 54.907 | 52.829 | 10.922 | 1.00 | 28.34 |
| ATOM | 3589 | NE2 | GLN | A | 469 | 57.074 | 52.746 | 10.216 | 1.00 | 23.42 |
| ATOM | 3590 | N | LEU | A | 470 | 54.496 | 47.825 | 8.619 | 1.00 | 28.25 |
| ATOM | 3591 | CA | LEU | A | 470 | 54.587 | 46.804 | 9.611 | 1.00 | 29.45 |
| ATOM | 3592 | C | LEU | A | 470 | 55.797 | 47.118 | 10.459 | 1.00 | 30.58 |
| ATOM | 3593 | O | LEU | A | 470 | 56.836 | 47.556 | 9.967 | 1.00 | 30.74 |
| ATOM | 3594 | CB | LEU | A | 470 | 54.777 | 45.416 | 8.997 | 1.00 | 29.96 |
| ATOM | 3595 | CG | LEU | A | 470 | 53.477 | 44.700 | 8.545 | 1.00 | 31.71 |
| ATOM | 3596 | CD1 | LEU | A | 470 | 53.812 | 43.418 | 7.817 | 1.00 | 32.62 |
| ATOM | 3597 | CD2 | LEU | A | 470 | 52.576 | 44.404 | 9.710 | 1.00 | 29.84 |
| ATOM | 3598 | N | ARG | A | 471 | 55.667 | 46.868 | 11.740 | 1.00 | 32.00 |
| ATOM | 3599 | CA | ARG | A | 471 | 56.770 | 47.057 | 12.635 | 1.00 | 33.21 |
| ATOM | 3600 | C | ARG | A | 471 | 56.856 | 45.811 | 13.476 | 1.00 | 32.36 |
| ATOM | 3601 | O | ARG | A | 471 | 55.922 | 45.448 | 14.189 | 1.00 | 31.00 |
| ATOM | 3602 | CB | ARG | A | 471 | 56.614 | 48.363 | 13.390 | 1.00 | 34.66 |
| ATOM | 3603 | CG | ARG | A | 471 | 55.836 | 48.362 | 14.607 | 1.00 | 40.62 |
| ATOM | 3604 | CD | ARG | A | 471 | 56.698 | 48.373 | 15.909 | 1.00 | 48.15 |
| ATOM | 3605 | NE | ARG | A | 471 | 55.770 | 48.361 | 17.041 | 1.00 | 53.40 |
| ATOM | 3606 | CZ | ARG | A | 471 | 55.725 | 49.259 | 18.013 | 1.00 | 58.18 |
| ATOM | 3607 | NH1 | ARG | A | 471 | 56.628 | 50.241 | 18.094 | 1.00 | 61.35 |
| ATOM | 3608 | NH2 | ARG | A | 471 | 54.785 | 49.148 | 18.937 | 1.00 | 58.39 |
| ATOM | 3609 | N | CYS | A | 472 | 57.929 | 45.069 | 13.232 | 1.00 | 31.04 |
| ATOM | 3610 | CA | CYS | A | 472 | 58.189 | 43.834 | 13.942 | 1.00 | 31.86 |
| ATOM | 3611 | C | CYS | A | 472 | 59.049 | 44.198 | 15.119 | 1.00 | 31.18 |
| ATOM | 3612 | O | CYS | A | 472 | 59.922 | 45.056 | 14.990 | 1.00 | 29.72 |
| ATOM | 3613 | CB | CYS | A | 472 | 58.876 | 42.844 | 12.993 | 1.00 | 32.72 |
| ATOM | 3614 | SG | CYS | A | 472 | 60.216 | 41.804 | 13.588 | 1.00 | 34.36 |
| ATOM | 3615 | N | SER | A | 473 | 58.755 | 43.566 | 16.248 | 1.00 | 30.57 |
| ATOM | 3616 | CA | SER | A | 473 | 59.372 | 43.858 | 17.532 | 1.00 | 31.00 |
| ATOM | 3617 | C | SER | A | 473 | 60.270 | 42.760 | 18.076 | 1.00 | 30.76 |
| ATOM | 3618 | O | SER | A | 473 | 60.819 | 42.915 | 19.159 | 1.00 | 32.46 |
| ATOM | 3619 | CB | SER | A | 473 | 58.260 | 44.077 | 18.588 | 1.00 | 31.69 |
| ATOM | 3620 | OG | SER | A | 473 | 57.860 | 45.417 | 18.636 | 1.00 | 32.84 |
| ATOM | 3621 | N | GLY | A | 474 | 60.402 | 41.644 | 17.385 | 1.00 | 30.01 |
| ATOM | 3622 | CA | GLY | A | 474 | 61.186 | 40.535 | 17.887 | 1.00 | 29.79 |
| ATOM | 3623 | C | GLY | A | 474 | 60.725 | 39.199 | 17.347 | 1.00 | 29.71 |
| ATOM | 3624 | O | GLY | A | 474 | 59.682 | 39.128 | 16.739 | 1.00 | 28.91 |
| ATOM | 3625 | N | PRO | A | 475 | 61.418 | 38.118 | 17.679 | 1.00 | 29.74 |
| ATOM | 3626 | CA | PRO | A | 475 | 62.509 | 38.114 | 18.663 | 1.00 | 30.22 |
| ATOM | 3627 | C | PRO | A | 475 | 63.829 | 38.661 | 18.147 | 1.00 | 30.24 |
| ATOM | 3628 | O | PRO | A | 475 | 64.712 | 38.778 | 18.969 | 1.00 | 31.04 |
| ATOM | 3629 | CB | PRO | A | 475 | 62.674 | 36.609 | 19.015 | 1.00 | 29.67 |
| ATOM | 3630 | CG | PRO | A | 475 | 61.922 | 35.843 | 17.911 | 1.00 | 29.65 |
| ATOM | 3631 | CD | PRO | A | 475 | 61.206 | 36.800 | 17.066 | 1.00 | 30.13 |
| ATOM | 3632 | N | GLY | A | 476 | 63.963 | 38.978 | 16.855 | 1.00 | 29.71 |
| ATOM | 3633 | CA | GLY | A | 476 | 65.211 | 39.512 | 16.314 | 1.00 | 29.41 |
| ATOM | 3634 | C | GLY | A | 476 | 65.119 | 41.006 | 16.463 | 1.00 | 30.15 |
| ATOM | 3635 | O | GLY | A | 476 | 64.185 | 41.506 | 17.120 | 1.00 | 30.30 |
| ATOM | 3636 | N | LEU | A | 477 | 66.067 | 41.727 | 15.882 | 1.00 | 29.02 |
| ATOM | 3637 | CA | LEU | A | 477 | 66.074 | 43.170 | 15.928 | 1.00 | 28.65 |
| ATOM | 3638 | C | LEU | A | 477 | 64.868 | 43.791 | 15.190 | 1.00 | 28.40 |
| ATOM | 3639 | O | LEU | A | 477 | 64.433 | 43.304 | 14.157 | 1.00 | 26.15 |
| ATOM | 3640 | CB | LEU | A | 477 | 67.372 | 43.679 | 15.293 | 1.00 | 28.42 |
| ATOM | 3641 | CG | LEU | A | 477 | 68.606 | 43.457 | 16.177 | 1.00 | 29.87 |
| ATOM | 3642 | CD1 | LEU | A | 477 | 69.808 | 43.864 | 15.412 | 1.00 | 31.27 |
| ATOM | 3643 | CD2 | LEU | A | 477 | 68.505 | 44.276 | 17.417 | 1.00 | 32.61 |
| ATOM | 3644 | N | PRO | A | 478 | 64.337 | 44.870 | 15.724 | 1.00 | 28.72 |
| ATOM | 3645 | CA | PRO | A | 478 | 63.171 | 45.506 | 15.103 | 1.00 | 28.98 |
| ATOM | 3646 | C | PRO | A | 478 | 63.419 | 45.825 | 13.638 | 1.00 | 28.98 |
| ATOM | 3647 | O | PRO | A | 478 | 64.550 | 46.155 | 13.244 | 1.00 | 27.85 |
| ATOM | 3648 | CB | PRO | A | 478 | 62.978 | 46.761 | 15.941 | 1.00 | 28.60 |
| ATOM | 3649 | CG | PRO | A | 478 | 63.414 | 46.249 | 17.350 | 1.00 | 29.75 |
| ATOM | 3650 | CD | PRO | A | 478 | 64.714 | 45.532 | 16.984 | 1.00 | 29.65 |
| ATOM | 3651 | N | LEU | A | 479 | 62.338 | 45.722 | 12.864 | 1.00 | 28.30 |
| ATOM | 3652 | CA | LEU | A | 479 | 62.375 | 45.825 | 11.412 | 1.00 | 27.04 |
| ATOM | 3653 | C | LEU | A | 479 | 61.045 | 46.453 | 10.988 | 1.00 | 26.54 |
| ATOM | 3654 | O | LEU | A | 479 | 59.962 | 45.958 | 11.325 | 1.00 | 23.86 |
| ATOM | 3655 | CB | LEU | A | 479 | 62.564 | 44.428 | 10.862 | 1.00 | 27.50 |
| ATOM | 3656 | CG | LEU | A | 479 | 62.972 | 44.059 | 9.438 | 1.00 | 28.18 |
| ATOM | 3657 | CD1 | LEU | A | 479 | 61.838 | 43.399 | 8.745 | 1.00 | 29.02 |
| ATOM | 3658 | CD2 | LEU | A | 479 | 63.598 | 45.165 | 8.607 | 1.00 | 29.05 |
| ATOM | 3659 | N | TYR | A | 480 | 61.172 | 47.592 | 10.317 | 1.00 | 25.53 |
| ATOM | 3660 | CA | TYR | A | 480 | 60.084 | 48.394 | 9.838 | 1.00 | 25.57 |
| ATOM | 3661 | C | TYR | A | 480 | 60.084 | 48.347 | 8.311 | 1.00 | 25.69 |
| ATOM | 3662 | O | TYR | A | 480 | 61.123 | 48.628 | 7.682 | 1.00 | 24.97 |
| ATOM | 3663 | CB | TYR | A | 480 | 60.307 | 49.839 | 10.334 | 1.00 | 26.20 |
| ATOM | 3664 | CG | TYR | A | 480 | 60.366 | 49.940 | 11.886 | 1.00 | 26.83 |
| ATOM | 3665 | CD1 | TYR | A | 480 | 61.473 | 49.507 | 12.582 | 1.00 | 26.40 |
| ATOM | 3666 | CD2 | TYR | A | 480 | 59.288 | 50.441 | 12.624 | 1.00 | 28.75 |
| ATOM | 3667 | CE1 | TYR | A | 480 | 61.541 | 49.597 | 13.959 | 1.00 | 27.86 |
| ATOM | 3668 | CE2 | TYR | A | 480 | 59.342 | 50.563 | 14.009 | 1.00 | 27.36 |
| ATOM | 3669 | CZ | TYR | A | 480 | 60.474 | 50.126 | 14.671 | 1.00 | 28.64 |
| ATOM | 3670 | OH | TYR | A | 480 | 60.589 | 50.175 | 16.028 | 1.00 | 32.30 |
| ATOM | 3671 | N | THR | A | 481 | 58.947 | 47.948 | 7.740 | 1.00 | 25.79 |
| ATOM | 3672 | CA | THR | A | 481 | 58.733 | 47.783 | 6.280 | 1.00 | 25.94 |
| ATOM | 3673 | C | THR | A | 481 | 57.444 | 48.464 | 5.823 | 1.00 | 26.79 |
| ATOM | 3674 | O | THR | A | 481 | 56.468 | 48.610 | 6.603 | 1.00 | 27.75 |
| ATOM | 3675 | CB | THR | A | 481 | 58.642 | 46.300 | 5.940 | 1.00 | 25.77 |
| ATOM | 3676 | OG1 | THR | A | 481 | 57.689 | 45.652 | 6.796 | 1.00 | 24.73 |
| ATOM | 3677 | CG2 | THR | A | 481 | 59.953 | 45.559 | 6.259 | 1.00 | 25.11 |
| ATOM | 3678 | N | LEU | A | 482 | 57.407 | 48.893 | 4.577 | 1.00 | 27.02 |
| ATOM | 3679 | CA | LEU | A | 482 | 56.198 | 49.551 | 4.017 | 1.00 | 28.39 |
| ATOM | 3680 | C | LEU | A | 482 | 55.617 | 48.647 | 2.970 | 1.00 | 29.00 |
| ATOM | 3681 | O | LEU | A | 482 | 56.364 | 47.964 | 2.261 | 1.00 | 28.88 |
| ATOM | 3682 | CB | LEU | A | 482 | 56.583 | 50.886 | 3.393 | 1.00 | 28.80 |
| ATOM | 3683 | CG | LEU | A | 482 | 55.694 | 52.065 | 3.061 | 1.00 | 29.45 |
| ATOM | 3684 | CD1 | LEU | A | 482 | 55.322 | 52.000 | 1.602 | 1.00 | 34.74 |
| ATOM | 3685 | CD2 | LEU | A | 482 | 54.559 | 52.139 | 4.010 | 1.00 | 31.91 |
| ATOM | 3686 | N | HIS | A | 483 | 54.286 | 48.625 | 2.888 | 1.00 | 29.13 |
| ATOM | 3687 | CA | HIS | A | 483 | 53.561 | 47.745 | 1.987 | 1.00 | 29.27 |
| ATOM | 3688 | C | HIS | A | 483 | 52.327 | 48.347 | 1.339 | 1.00 | 29.70 |
| ATOM | 3689 | O | HIS | A | 483 | 51.631 | 49.183 | 1.928 | 1.00 | 29.62 |
| ATOM | 3690 | CB | HIS | A | 483 | 53.058 | 46.565 | 2.774 | 1.00 | 29.56 |
| ATOM | 3691 | CG | HIS | A | 483 | 54.109 | 45.781 | 3.488 | 1.00 | 30.37 |
| ATOM | 3692 | ND1 | HIS | A | 483 | 54.478 | 46.048 | 4.791 | 1.00 | 32.06 |
| ATOM | 3693 | CD2 | HIS | A | 483 | 54.809 | 44.682 | 3.121 | 1.00 | 29.51 |
| ATOM | 3694 | CE1 | HIS | A | 483 | 55.394 | 45.179 | 5.172 | 1.00 | 28.87 |
| ATOM | 3695 | NE2 | HIS | A | 483 | 55.614 | 44.340 | 4.181 | 1.00 | 29.23 |
| ATOM | 3696 | N | SER | A | 484 | 52.017 | 47.866 | 0.140 | 1.00 | 30.49 |
| ATOM | 3697 | CA | SER | A | 484 | 50.780 | 48.273 | −0.530 | 1.00 | 31.66 |
| ATOM | 3698 | C | SER | A | 484 | 49.644 | 47.255 | −0.340 | 1.00 | 31.04 |
| ATOM | 3699 | O | SER | A | 484 | 49.830 | 46.068 | −0.516 | 1.00 | 29.45 |
| ATOM | 3700 | CB | SER | A | 484 | 51.018 | 48.473 | −2.004 | 1.00 | 31.81 |
| ATOM | 3701 | OG | SER | A | 484 | 51.194 | 47.221 | −2.593 | 1.00 | 36.07 |
| ATOM | 3702 | N | SER | A | 485 | 48.471 | 47.713 | 0.052 | 1.00 | 31.59 |
| ATOM | 3703 | CA | SER | A | 485 | 47.377 | 46.779 | 0.341 | 1.00 | 31.97 |
| ATOM | 3704 | C | SER | A | 485 | 46.812 | 45.981 | −0.889 | 1.00 | 33.22 |
| ATOM | 3705 | O | SER | A | 485 | 46.347 | 44.871 | −0.733 | 1.00 | 31.17 |
| ATOM | 3706 | CB | SER | A | 485 | 46.224 | 47.517 | 1.056 | 1.00 | 31.84 |
| ATOM | 3707 | OG | SER | A | 485 | 46.495 | 47.820 | 2.429 | 1.00 | 30.78 |
| ATOM | 3708 | N | VAL | A | 486 | 46.906 | 46.520 | −2.092 | 1.00 | 35.73 |
| ATOM | 3709 | CA | VAL | A | 486 | 46.320 | 45.843 | −3.290 | 1.00 | 38.83 |
| ATOM | 3710 | C | VAL | A | 486 | 46.643 | 44.361 | −3.445 | 1.00 | 39.66 |
| ATOM | 3711 | O | VAL | A | 486 | 45.762 | 43.547 | −3.497 | 1.00 | 39.83 |
| ATOM | 3712 | CB | VAL | A | 486 | 46.779 | 46.461 | −4.616 | 1.00 | 39.39 |
| ATOM | 3713 | CG1 | VAL | A | 486 | 45.994 | 45.827 | −5.743 | 1.00 | 40.77 |
| ATOM | 3714 | CG2 | VAL | A | 486 | 46.560 | 47.936 | −4.622 | 1.00 | 40.89 |
| ATOM | 3715 | N | ASN | A | 487 | 47.918 | 44.043 | −3.544 | 1.00 | 41.49 |
| ATOM | 3716 | CA | ASN | A | 487 | 48.397 | 42.673 | −3.672 | 1.00 | 43.70 |
| ATOM | 3717 | C | ASN | A | 487 | 49.507 | 42.699 | −2.646 | 1.00 | 44.54 |
| ATOM | 3718 | O | ASN | A | 487 | 50.661 | 43.041 | −2.976 | 1.00 | 47.53 |
| ATOM | 3719 | CB | ASN | A | 487 | 48.968 | 42.424 | −5.079 | 1.00 | 43.60 |
| ATOM | 3720 | CG | ASN | A | 487 | 47.931 | 42.677 | −6.209 | 1.00 | 46.15 |
| ATOM | 3721 | OD1 | ASN | A | 487 | 47.879 | 43.771 | −6.808 | 1.00 | 45.78 |
| ATOM | 3722 | ND2 | ASN | A | 487 | 47.090 | 41.666 | −6.480 | 1.00 | 47.33 |
| ATOM | 3723 | N | ASP | A | 488 | 49.156 | 42.406 | −1.407 | 1.00 | 44.16 |
| ATOM | 3724 | CA | ASP | A | 488 | 49.991 | 42.765 | −0.256 | 1.00 | 43.13 |
| ATOM | 3725 | C | ASP | A | 488 | 51.440 | 42.640 | −0.572 | 1.00 | 42.07 |
| ATOM | 3726 | O | ASP | A | 488 | 52.063 | 41.698 | −0.129 | 1.00 | 41.39 |
| ATOM | 3727 | CB | ASP | A | 488 | 49.632 | 41.919 | 0.969 | 1.00 | 43.40 |
| ATOM | 3728 | CG | ASP | A | 488 | 48.261 | 42.269 | 1.535 | 1.00 | 44.42 |
| ATOM | 3729 | OD1 | ASP | A | 488 | 47.250 | 41.758 | 1.016 | 1.00 | 44.23 |
| ATOM | 3730 | OD2 | ASP | A | 488 | 48.113 | 43.048 | 2.498 | 1.00 | 47.17 |
| ATOM | 3731 | N | LYS | A | 489 | 51.970 | 43.591 | −1.336 | 1.00 | 41.40 |
| ATOM | 3732 | CA | LYS | A | 489 | 53.362 | 43.532 | −1.778 | 1.00 | 41.77 |
| ATOM | 3733 | C | LYS | A | 489 | 54.208 | 44.372 | −0.850 | 1.00 | 39.87 |
| ATOM | 3734 | O | LYS | A | 489 | 53.766 | 45.428 | −0.402 | 1.00 | 39.61 |
| ATOM | 3735 | CB | LYS | A | 489 | 53.509 | 44.059 | −3.223 | 1.00 | 43.04 |
| ATOM | 3736 | CG | LYS | A | 489 | 54.927 | 43.896 | −3.868 | 1.00 | 45.55 |
| ATOM | 3737 | CD | LYS | A | 489 | 54.887 | 44.193 | −5.389 | 1.00 | 48.94 |
| ATOM | 3738 | CE | LYS | A | 489 | 56.304 | 44.463 | −6.036 | 1.00 | 51.28 |
| ATOM | 3739 | NZ | LYS | A | 489 | 56.810 | 45.920 | −6.081 | 1.00 | 49.79 |
| ATOM | 3740 | N | GLY | A | 490 | 55.396 | 43.882 | −0.532 | 1.00 | 37.79 |
| ATOM | 3741 | CA | GLY | A | 490 | 56.324 | 44.656 | 0.259 | 1.00 | 37.25 |
| ATOM | 3742 | C | GLY | A | 490 | 56.914 | 45.694 | −0.665 | 1.00 | 36.45 |
| ATOM | 3743 | O | GLY | A | 490 | 57.299 | 45.383 | −1.788 | 1.00 | 37.78 |
| ATOM | 3744 | N | LEU | A | 491 | 56.948 | 46.942 | −0.240 | 1.00 | 35.33 |
| ATOM | 3745 | CA | LEU | A | 491 | 57.461 | 47.964 | −1.104 | 1.00 | 34.44 |
| ATOM | 3746 | C | LEU | A | 491 | 58.935 | 48.323 | −0.856 | 1.00 | 34.34 |
| ATOM | 3747 | O | LEU | A | 491 | 59.670 | 48.514 | −1.839 | 1.00 | 34.89 |
| ATOM | 3748 | CB | LEU | A | 491 | 56.580 | 49.197 | −0.989 | 1.00 | 34.55 |
| ATOM | 3749 | CG | LEU | A | 491 | 55.123 | 49.022 | −1.451 | 1.00 | 34.62 |
| ATOM | 3750 | CD1 | LEU | A | 491 | 54.288 | 50.122 | −0.858 | 1.00 | 33.61 |
| ATOM | 3751 | CD2 | LEU | A | 491 | 54.968 | 48.996 | −2.978 | 1.00 | 31.54 |
| ATOM | 3752 | N | ARG | A | 492 | 59.343 | 48.434 | 0.426 | 1.00 | 32.66 |
| ATOM | 3753 | CA | ARG | A | 492 | 60.660 | 48.927 | 0.871 | 1.00 | 31.58 |
| ATOM | 3754 | C | ARG | A | 492 | 60.864 | 48.546 | 2.277 | 1.00 | 30.93 |
| ATOM | 3755 | O | ARG | A | 492 | 59.901 | 48.480 | 3.019 | 1.00 | 30.06 |
| ATOM | 3756 | CB | ARG | A | 492 | 60.645 | 50.434 | 1.042 | 1.00 | 32.97 |
| ATOM | 3757 | CG | ARG | A | 492 | 60.828 | 51.171 | −0.161 | 1.00 | 34.92 |
| ATOM | 3758 | CD | ARG | A | 492 | 60.326 | 52.610 | −0.150 | 1.00 | 34.50 |
| ATOM | 3759 | NE | ARG | A | 492 | 59.490 | 52.636 | −1.321 | 1.00 | 32.59 |
| ATOM | 3760 | CZ | ARG | A | 492 | 58.219 | 52.906 | −1.332 | 1.00 | 34.65 |
| ATOM | 3761 | NH1 | ARG | A | 492 | 57.575 | 53.305 | −0.232 | 1.00 | 32.87 |
| ATOM | 3762 | NH2 | ARG | A | 492 | 57.586 | 52.809 | −2.491 | 1.00 | 37.06 |
| ATOM | 3763 | N | VAL | A | 493 | 62.127 | 48.449 | 2.653 | 1.00 | 30.19 |
| ATOM | 3764 | CA | VAL | A | 493 | 62.542 | 48.283 | 4.028 | 1.00 | 30.35 |
| ATOM | 3765 | C | VAL | A | 493 | 62.731 | 49.718 | 4.529 | 1.00 | 29.81 |
| ATOM | 3766 | O | VAL | A | 493 | 63.407 | 50.499 | 3.881 | 1.00 | 29.10 |
| ATOM | 3767 | CB | VAL | A | 493 | 63.861 | 47.525 | 4.125 | 1.00 | 30.28 |
| ATOM | 3768 | CG1 | VAL | A | 493 | 64.339 | 47.465 | 5.601 | 1.00 | 32.53 |
| ATOM | 3769 | CG2 | VAL | A | 493 | 63.706 | 46.116 | 3.602 | 1.00 | 30.23 |
| ATOM | 3770 | N | LEU | A | 494 | 62.112 | 50.085 | 5.653 | 1.00 | 29.29 |
| ATOM | 3771 | CA | LEU | A | 494 | 62.266 | 51.451 | 6.148 | 1.00 | 27.76 |
| ATOM | 3772 | C | LEU | A | 494 | 63.412 | 51.601 | 7.157 | 1.00 | 27.82 |
| ATOM | 3773 | O | LEU | A | 494 | 64.179 | 52.547 | 7.086 | 1.00 | 26.89 |
| ATOM | 3774 | CB | LEU | A | 494 | 60.947 | 51.946 | 6.728 | 1.00 | 28.18 |
| ATOM | 3775 | CG | LEU | A | 494 | 59.767 | 51.913 | 5.744 | 1.00 | 25.32 |
| ATOM | 3776 | CD1 | LEU | A | 494 | 58.460 | 52.048 | 6.574 | 1.00 | 25.03 |
| ATOM | 3777 | CD2 | LEU | A | 494 | 59.837 | 53.020 | 4.700 | 1.00 | 25.99 |
| ATOM | 3778 | N | GLU | A | 495 | 63.507 | 50.668 | 8.097 | 1.00 | 26.93 |
| ATOM | 3779 | CA | GLU | A | 495 | 64.583 | 50.634 | 9.053 | 1.00 | 26.86 |
| ATOM | 3780 | C | GLU | A | 495 | 64.800 | 49.191 | 9.423 | 1.00 | 27.38 |
| ATOM | 3781 | O | GLU | A | 495 | 63.850 | 48.505 | 9.886 | 1.00 | 27.45 |
| ATOM | 3782 | CB | GLU | A | 495 | 64.227 | 51.421 | 10.320 | 1.00 | 26.93 |
| ATOM | 3783 | CG | GLU | A | 495 | 65.284 | 51.279 | 11.406 | 1.00 | 27.46 |
| ATOM | 3784 | CD | GLU | A | 495 | 66.667 | 51.737 | 10.964 | 1.00 | 28.72 |
| ATOM | 3785 | OE1 | GLU | A | 495 | 66.849 | 52.939 | 10.662 | 1.00 | 27.14 |
| ATOM | 3786 | OE2 | GLU | A | 495 | 67.565 | 50.872 | 10.896 | 1.00 | 32.40 |
| ATOM | 3787 | N | ASP | A | 496 | 66.021 | 48.710 | 9.216 | 1.00 | 26.60 |
| ATOM | 3788 | CA | ASP | A | 496 | 66.343 | 47.347 | 9.595 | 1.00 | 27.19 |
| ATOM | 3789 | C | ASP | A | 496 | 67.312 | 47.209 | 10.761 | 1.00 | 26.76 |
| ATOM | 3790 | O | ASP | A | 496 | 67.664 | 46.094 | 11.095 | 1.00 | 25.91 |
| ATOM | 3791 | CB | ASP | A | 496 | 66.845 | 46.509 | 8.409 | 1.00 | 27.04 |
| ATOM | 3792 | CG | ASP | A | 496 | 68.163 | 46.991 | 7.848 | 1.00 | 30.52 |
| ATOM | 3793 | OD1 | ASP | A | 496 | 68.807 | 47.907 | 8.429 | 1.00 | 35.11 |
| ATOM | 3794 | OD2 | ASP | A | 496 | 68.622 | 46.504 | 6.788 | 1.00 | 32.05 |
| ATOM | 3795 | N | ASN | A | 497 | 67.705 | 48.316 | 11.377 | 1.00 | 27.15 |
| ATOM | 3796 | CA | ASN | A | 497 | 68.661 | 48.285 | 12.494 | 1.00 | 28.78 |
| ATOM | 3797 | C | ASN | A | 497 | 69.958 | 47.515 | 12.182 | 1.00 | 29.53 |
| ATOM | 3798 | O | ASN | A | 497 | 70.514 | 46.820 | 13.063 | 1.00 | 29.16 |
| ATOM | 3799 | CB | ASN | A | 497 | 67.977 | 47.719 | 13.784 | 1.00 | 29.45 |
| ATOM | 3800 | CG | ASN | A | 497 | 67.124 | 48.763 | 14.482 | 1.00 | 30.00 |
| ATOM | 3801 | OD1 | ASN | A | 497 | 67.646 | 49.769 | 14.931 | 1.00 | 29.24 |
| ATOM | 3802 | ND2 | ASN | A | 497 | 65.789 | 48.545 | 14.541 | 1.00 | 29.26 |
| ATOM | 3803 | N | SER | A | 498 | 70.432 | 47.622 | 10.936 | 1.00 | 29.99 |
| ATOM | 3804 | CA | SER | A | 498 | 71.712 | 47.020 | 10.552 | 1.00 | 31.47 |
| ATOM | 3805 | C | SER | A | 498 | 72.841 | 47.552 | 11.438 | 1.00 | 31.80 |
| ATOM | 3806 | O | SER | A | 498 | 73.730 | 46.819 | 11.855 | 1.00 | 32.35 |
| ATOM | 3807 | CB | SER | A | 498 | 72.055 | 47.346 | 9.088 | 1.00 | 31.18 |
| ATOM | 3808 | OG | SER | A | 498 | 72.035 | 48.768 | 8.935 | 1.00 | 33.29 |
| ATOM | 3809 | N | ALA | A | 499 | 72.798 | 48.834 | 11.730 | 1.00 | 32.02 |
| ATOM | 3810 | CA | ALA | A | 499 | 73.828 | 49.425 | 12.546 | 1.00 | 31.95 |
| ATOM | 3811 | C | ALA | A | 499 | 73.919 | 48.707 | 13.900 | 1.00 | 33.04 |
| ATOM | 3812 | O | ALA | A | 499 | 74.989 | 48.185 | 14.270 | 1.00 | 33.43 |
| ATOM | 3813 | CB | ALA | A | 499 | 73.590 | 50.888 | 12.681 | 1.00 | 31.12 |
| ATOM | 3814 | N | LEU | A | 500 | 72.794 | 48.580 | 14.605 | 1.00 | 33.98 |
| ATOM | 3815 | CA | LEU | A | 500 | 72.779 | 47.942 | 15.907 | 1.00 | 34.37 |
| ATOM | 3816 | C | LEU | A | 500 | 73.212 | 46.496 | 15.818 | 1.00 | 34.56 |
| ATOM | 3817 | O | LEU | A | 500 | 73.939 | 45.975 | 16.662 | 1.00 | 34.65 |
| ATOM | 3818 | CB | LEU | A | 500 | 71.365 | 48.041 | 16.494 | 1.00 | 35.16 |
| ATOM | 3819 | CG | LEU | A | 500 | 71.074 | 47.771 | 17.973 | 1.00 | 36.71 |
| ATOM | 3820 | CD1 | LEU | A | 500 | 70.484 | 46.432 | 18.084 | 1.00 | 38.33 |
| ATOM | 3821 | CD2 | LEU | A | 500 | 72.261 | 47.905 | 18.963 | 1.00 | 36.14 |
| ATOM | 3822 | N | ASP | A | 501 | 72.719 | 45.816 | 14.819 | 1.00 | 35.24 |
| ATOM | 3823 | CA | ASP | A | 501 | 73.139 | 44.443 | 14.585 | 1.00 | 36.88 |
| ATOM | 3824 | C | ASP | A | 501 | 74.696 | 44.349 | 14.649 | 1.00 | 36.67 |
| ATOM | 3825 | O | ASP | A | 501 | 75.249 | 43.495 | 15.355 | 1.00 | 35.36 |
| ATOM | 3826 | CB | ASP | A | 501 | 72.671 | 43.974 | 13.210 | 1.00 | 37.03 |
| ATOM | 3827 | CG | ASP | A | 501 | 72.683 | 42.495 | 13.088 | 1.00 | 41.29 |
| ATOM | 3828 | OD1 | ASP | A | 501 | 71.921 | 41.847 | 13.829 | 1.00 | 47.04 |
| ATOM | 3829 | OD2 | ASP | A | 501 | 73.420 | 41.864 | 12.295 | 1.00 | 48.28 |
| ATOM | 3830 | N | LYS | A | 502 | 75.404 | 45.224 | 13.936 | 1.00 | 36.25 |
| ATOM | 3831 | CA | LYS | A | 502 | 76.877 | 45.081 | 13.949 | 1.00 | 36.92 |
| ATOM | 3832 | C | LYS | A | 502 | 77.493 | 45.344 | 15.326 | 1.00 | 35.12 |
| ATOM | 3833 | O | LYS | A | 502 | 78.362 | 44.624 | 15.719 | 1.00 | 34.06 |
| ATOM | 3834 | CB | LYS | A | 502 | 77.591 | 45.872 | 12.839 | 1.00 | 37.57 |
| ATOM | 3835 | CG | LYS | A | 502 | 77.079 | 47.247 | 12.631 | 1.00 | 40.85 |
| ATOM | 3836 | CD | LYS | A | 502 | 78.126 | 48.152 | 11.978 | 1.00 | 45.63 |
| ATOM | 3837 | CE | LYS | A | 502 | 78.246 | 49.438 | 12.847 | 1.00 | 46.85 |
| ATOM | 3838 | NZ | LYS | A | 502 | 76.961 | 49.683 | 13.597 | 1.00 | 42.88 |
| ATOM | 3839 | N | MET | A | 503 | 76.995 | 46.309 | 16.087 | 1.00 | 34.82 |
| ATOM | 3840 | CA | MET | A | 503 | 77.579 | 46.538 | 17.380 | 1.00 | 35.39 |
| ATOM | 3841 | C | MET | A | 503 | 77.372 | 45.336 | 18.285 | 1.00 | 35.75 |
| ATOM | 3842 | O | MET | A | 503 | 78.279 | 44.940 | 19.019 | 1.00 | 35.08 |
| ATOM | 3843 | CB | MET | A | 503 | 77.014 | 47.788 | 18.017 | 1.00 | 35.70 |
| ATOM | 3844 | CG | MET | A | 503 | 77.302 | 49.022 | 17.252 | 1.00 | 37.65 |
| ATOM | 3845 | SD | MET | A | 503 | 76.521 | 50.415 | 18.018 | 1.00 | 43.87 |
| ATOM | 3846 | CE | MET | A | 503 | 74.863 | 50.228 | 17.447 | 1.00 | 44.49 |
| ATOM | 3847 | N | LEU | A | 504 | 76.187 | 44.735 | 18.181 | 1.00 | 36.04 |
| ATOM | 3848 | CA | LEU | A | 504 | 75.781 | 43.655 | 19.060 | 1.00 | 35.94 |
| ATOM | 3849 | C | LED | A | 504 | 76.558 | 42.406 | 18.780 | 1.00 | 36.06 |
| ATOM | 3850 | O | LEU | A | 504 | 76.739 | 41.551 | 19.664 | 1.00 | 35.69 |
| ATOM | 3851 | CB | LEU | A | 504 | 74.274 | 43.451 | 18.967 | 1.00 | 36.22 |
| ATOM | 3852 | CG | LEU | A | 504 | 73.461 | 44.615 | 19.567 | 1.00 | 37.88 |
| ATOM | 3853 | CD1 | LEU | A | 504 | 71.989 | 44.234 | 19.691 | 1.00 | 40.39 |
| ATOM | 3854 | CD2 | LEU | A | 504 | 73.950 | 45.071 | 20.940 | 1.00 | 37.32 |
| ATOM | 3855 | N | GLN | A | 505 | 77.069 | 42.308 | 17.557 | 1.00 | 36.55 |
| ATOM | 3856 | CA | GLN | A | 505 | 77.963 | 41.218 | 17.220 | 1.00 | 36.93 |
| ATOM | 3857 | C | GLN | A | 505 | 79.177 | 41.269 | 18.157 | 1.00 | 35.99 |
| ATOM | 3858 | O | GLN | A | 505 | 79.738 | 40.237 | 18.463 | 1.00 | 34.66 |
| ATOM | 3859 | CB | GLN | A | 505 | 78.407 | 41.308 | 15.752 | 1.00 | 37.71 |
| ATOM | 3860 | CG | GLN | A | 505 | 77.323 | 40.933 | 14.739 | 1.00 | 41.41 |
| ATOM | 3861 | CD | GLN | A | 505 | 77.637 | 41.489 | 13.330 | 1.00 | 47.47 |
| ATOM | 3862 | OE1 | GLN | A | 505 | 78.811 | 41.591 | 12.958 | 1.00 | 52.31 |
| ATOM | 3863 | NE2 | GLN | A | 505 | 76.596 | 41.866 | 12.566 | 1.00 | 48.31 |
| ATOM | 3864 | N | ASN | A | 506 | 79.584 | 42.457 | 18.605 | 1.00 | 36.22 |
| ATOM | 3865 | CA | ASN | A | 506 | 80.733 | 42.539 | 19.507 | 1.00 | 37.72 |
| ATOM | 3866 | C | ASN | A | 506 | 80.348 | 42.595 | 20.978 | 1.00 | 37.33 |
| ATOM | 3867 | O | ASN | A | 506 | 81.134 | 43.044 | 21.804 | 1.00 | 36.99 |
| ATOM | 3868 | CB | ASN | A | 506 | 81.685 | 43.699 | 19.196 | 1.00 | 38.48 |
| ATOM | 3869 | CG | ASN | A | 506 | 83.156 | 43.382 | 19.579 | 1.00 | 41.29 |
| ATOM | 3870 | OD1 | ASN | A | 506 | 83.488 | 42.260 | 20.002 | 1.00 | 46.82 |
| ATOM | 3871 | ND2 | ASN | A | 506 | 84.038 | 44.348 | 19.386 | 1.00 | 44.80 |
| ATOM | 3872 | N | VAL | A | 507 | 79.158 | 42.105 | 21.309 | 1.00 | 37.21 |
| ATOM | 3873 | CA | VAL | A | 507 | 78.777 | 42.019 | 22.697 | 1.00 | 36.18 |
| ATOM | 3874 | C | VAL | A | 507 | 78.353 | 40.601 | 23.064 | 1.00 | 35.75 |
| ATOM | 3875 | O | VAL | A | 507 | 77.729 | 39.885 | 22.272 | 1.00 | 33.24 |
| ATOM | 3876 | CB | VAL | A | 507 | 77.672 | 43.043 | 23.009 | 1.00 | 36.32 |
| ATOM | 3877 | CG1 | VAL | A | 507 | 77.465 | 43.154 | 24.527 | 1.00 | 36.71 |
| ATOM | 3878 | CG2 | VAL | A | 507 | 78.055 | 44.354 | 22.479 | 1.00 | 33.94 |
| ATOM | 3879 | N | GLN | A | 508 | 78.723 | 40.155 | 24.262 | 1.00 | 35.82 |
| ATOM | 3880 | CA | GLN | A | 508 | 78.250 | 38.829 | 24.703 | 1.00 | 36.41 |
| ATOM | 3881 | C | GLN | A | 508 | 76.760 | 38.960 | 25.132 | 1.00 | 36.27 |
| ATOM | 3882 | O | GLN | A | 508 | 76.448 | 39.117 | 26.317 | 1.00 | 37.04 |
| ATOM | 3883 | CB | GLN | A | 508 | 79.101 | 38.277 | 25.861 | 1.00 | 36.81 |
| ATOM | 3884 | CG | GLN | A | 508 | 80.602 | 38.172 | 25.575 | 1.00 | 36.67 |
| ATOM | 3885 | CD | GLN | A | 508 | 81.371 | 37.494 | 26.673 | 1.00 | 34.39 |
| ATOM | 3886 | OE1 | GLN | A | 508 | 82.271 | 38.098 | 27.272 | 1.00 | 37.16 |
| ATOM | 3887 | NE2 | GLN | A | 508 | 81.082 | 36.222 | 26.906 | 1.00 | 32.55 |
| ATOM | 3888 | N | MET | A | 509 | 75.851 | 38.873 | 24.172 | 1.00 | 35.31 |
| ATOM | 3889 | CA | MET | A | 509 | 74.428 | 39.078 | 24.429 | 1.00 | 35.41 |
| ATOM | 3890 | C | MET | A | 509 | 73.742 | 37.843 | 24.986 | 1.00 | 34.31 |
| ATOM | 3891 | O | MET | A | 509 | 74.036 | 36.754 | 24.543 | 1.00 | 33.81 |
| ATOM | 3892 | CB | MET | A | 509 | 73.741 | 39.499 | 23.125 | 1.00 | 35.57 |
| ATOM | 3893 | CG | MET | A | 509 | 74.126 | 40.891 | 22.688 | 1.00 | 38.49 |
| ATOM | 3894 | SD | MET | A | 509 | 73.589 | 42.110 | 23.958 | 1.00 | 44.97 |
| ATOM | 3895 | CE | MET | A | 509 | 71.763 | 41.920 | 23.553 | 1.00 | 40.97 |
| ATOM | 3896 | N | PRO | A | 510 | 72.811 | 38.009 | 25.935 | 1.00 | 33.30 |
| ATOM | 3897 | CA | PRO | A | 510 | 72.090 | 36.869 | 26.499 | 1.00 | 33.10 |
| ATOM | 3898 | C | PRO | A | 510 | 71.177 | 36.307 | 25.465 | 1.00 | 33.21 |
| ATOM | 3899 | O | PRO | A | 510 | 70.964 | 36.980 | 24.485 | 1.00 | 32.54 |
| ATOM | 3900 | CB | PRO | A | 510 | 71.163 | 37.501 | 27.580 | 1.00 | 33.42 |
| ATOM | 3901 | CG | PRO | A | 510 | 71.103 | 38.917 | 27.314 | 1.00 | 32.83 |
| ATOM | 3902 | CD | PRO | A | 510 | 72.326 | 39.288 | 26.467 | 1.00 | 32.45 |
| ATOM | 3903 | N | SER | A | 511 | 70.589 | 35.145 | 25.706 | 1.00 | 33.60 |
| ATOM | 3904 | CA | SER | A | 511 | 69.579 | 34.638 | 24.807 | 1.00 | 34.17 |
| ATOM | 3905 | C | SER | A | 511 | 68.271 | 34.653 | 25.565 | 1.00 | 34.99 |
| ATOM | 3906 | O | SER | A | 511 | 68.233 | 34.799 | 26.804 | 1.00 | 34.35 |
| ATOM | 3907 | CB | SER | A | 511 | 69.864 | 33.201 | 24.377 | 1.00 | 34.66 |
| ATOM | 3908 | OG | SER | A | 511 | 69.992 | 32.345 | 25.508 | 1.00 | 34.37 |
| ATOM | 3909 | N | LYS | A | 512 | 67.201 | 34.477 | 24.816 | 1.00 | 34.20 |
| ATOM | 3910 | CA | LYS | A | 512 | 65.893 | 34.427 | 25.396 | 1.00 | 34.13 |
| ATOM | 3911 | C | LYS | A | 512 | 65.214 | 33.122 | 25.035 | 1.00 | 33.91 |
| ATOM | 3912 | O | LYS | A | 512 | 65.164 | 32.716 | 23.866 | 1.00 | 32.89 |
| ATOM | 3913 | CB | LYS | A | 512 | 65.107 | 35.619 | 24.916 | 1.00 | 33.77 |
| ATOM | 3914 | CG | LYS | A | 512 | 63.673 | 35.564 | 25.266 | 1.00 | 33.43 |
| ATOM | 3915 | CD | LYS | A | 512 | 63.040 | 36.902 | 24.913 | 1.00 | 33.10 |
| ATOM | 3916 | CE | LYS | A | 512 | 61.708 | 37.119 | 25.555 | 1.00 | 29.91 |
| ATOM | 3917 | NZ | LYS | A | 512 | 61.085 | 38.350 | 24.967 | 1.00 | 28.32 |
| ATOM | 3918 | N | LYS | A | 513 | 64.699 | 32.471 | 26.058 | 1.00 | 33.75 |
| ATOM | 3919 | CA | LYS | A | 513 | 63.992 | 31.219 | 25.916 | 1.00 | 34.76 |
| ATOM | 3920 | C | LYS | A | 513 | 62.537 | 31.497 | 26.238 | 1.00 | 34.65 |
| ATOM | 3921 | O | LYS | A | 513 | 62.257 | 32.062 | 27.276 | 1.00 | 34.59 |
| ATOM | 3922 | CB | LYS | A | 513 | 64.575 | 30.201 | 26.889 | 1.00 | 35.33 |
| ATOM | 3923 | CG | LYS | A | 513 | 64.510 | 28.739 | 26.398 | 1.00 | 39.42 |
| ATOM | 3924 | CD | LYS | A | 513 | 63.413 | 27.898 | 27.072 | 1.00 | 41.17 |
| ATOM | 3925 | CE | LYS | A | 513 | 63.589 | 26.380 | 26.788 | 1.00 | 43.57 |
| ATOM | 3926 | NZ | LYS | A | 513 | 64.157 | 25.601 | 27.953 | 1.00 | 45.70 |
| ATOM | 3927 | N | LEU | A | 514 | 61.621 | 31.118 | 25.344 | 1.00 | 34.65 |
| ATOM | 3928 | CA | LEU | A | 514 | 60.191 | 31.352 | 25.505 | 1.00 | 34.56 |
| ATOM | 3929 | C | LEU | A | 514 | 59.563 | 29.987 | 25.383 | 1.00 | 35.35 |
| ATOM | 3930 | O | LEU | A | 514 | 59.745 | 29.340 | 24.381 | 1.00 | 34.58 |
| ATOM | 3931 | CB | LEU | A | 514 | 59.676 | 32.293 | 24.396 | 1.00 | 34.74 |
| ATOM | 3932 | CG | LEU | A | 514 | 58.176 | 32.603 | 24.293 | 1.00 | 34.11 |
| ATOM | 3933 | CD1 | LEU | A | 514 | 57.816 | 33.292 | 23.019 | 1.00 | 32.68 |
| ATOM | 3934 | CD2 | LEU | A | 514 | 57.357 | 31.351 | 24.362 | 1.00 | 35.00 |
| ATOM | 3935 | N | ASP | A | 515 | 58.784 | 29.555 | 26.374 | 1.00 | 36.47 |
| ATOM | 3936 | CA | ASP | A | 515 | 58.292 | 28.194 | 26.393 | 1.00 | 37.43 |
| ATOM | 3937 | C | ASP | A | 515 | 57.200 | 28.147 | 27.455 | 1.00 | 38.11 |
| ATOM | 3938 | O | ASP | A | 515 | 56.814 | 29.193 | 27.991 | 1.00 | 38.35 |
| ATOM | 3939 | CB | ASP | A | 515 | 59.456 | 27.286 | 26.789 | 1.00 | 38.01 |
| ATOM | 3940 | CG | ASP | A | 515 | 59.343 | 25.856 | 26.271 | 1.00 | 39.55 |
| ATOM | 3941 | OD1 | ASP | A | 515 | 58.247 | 25.307 | 26.003 | 1.00 | 40.04 |
| ATOM | 3942 | OD2 | ASP | A | 515 | 60.367 | 25.168 | 26.131 | 1.00 | 46.00 |
| ATOM | 3943 | N | PHE | A | 516 | 56.757 | 26.944 | 27.796 | 1.00 | 37.99 |
| ATOM | 3944 | CA | PHE | A | 516 | 55.673 | 26.763 | 28.721 | 1.00 | 39.35 |
| ATOM | 3945 | C | PHE | A | 516 | 55.867 | 25.545 | 29.646 | 1.00 | 40.21 |
| ATOM | 3946 | O | PHE | A | 516 | 56.629 | 24.643 | 29.343 | 1.00 | 40.04 |
| ATOM | 3947 | CB | PHE | A | 516 | 54.363 | 26.612 | 27.924 | 1.00 | 39.53 |
| ATOM | 3948 | CG | PHE | A | 516 | 54.296 | 25.371 | 27.028 | 1.00 | 39.01 |
| ATOM | 3949 | CD1 | PHE | A | 516 | 53.835 | 24.148 | 27.536 | 1.00 | 41.98 |
| ATOM | 3950 | CD2 | PHE | A | 516 | 54.612 | 25.451 | 25.677 | 1.00 | 39.54 |
| ATOM | 3951 | CE1 | PHE | A | 516 | 53.735 | 23.004 | 26.707 | 1.00 | 41.97 |
| ATOM | 3952 | CE2 | PHE | A | 516 | 54.510 | 24.324 | 24.817 | 1.00 | 40.66 |
| ATOM | 3953 | CZ | PHE | A | 516 | 54.072 | 23.102 | 25.331 | 1.00 | 41.88 |
| ATOM | 3954 | N | ILE | A | 517 | 55.183 | 25.561 | 30.777 | 1.00 | 41.13 |
| ATOM | 3955 | CA | ILE | A | 517 | 55.081 | 24.403 | 31.646 | 1.00 | 42.46 |
| ATOM | 3956 | C | ILE | A | 517 | 53.589 | 24.075 | 31.754 | 1.00 | 43.86 |
| ATOM | 3957 | O | ILE | A | 517 | 52.729 | 24.922 | 31.461 | 1.00 | 43.22 |
| ATOM | 3958 | CB | ILE | A | 517 | 55.692 | 24.669 | 33.035 | 1.00 | 42.62 |
| ATOM | 3959 | CG1 | ILE | A | 517 | 54.909 | 25.757 | 33.752 | 1.00 | 43.03 |
| ATOM | 3960 | CG2 | ILE | A | 517 | 57.195 | 25.002 | 32.910 | 1.00 | 42.66 |
| ATOM | 3961 | CD1 | ILE | A | 517 | 55.637 | 26.398 | 34.873 | 1.00 | 43.74 |
| ATOM | 3962 | N | ILE | A | 518 | 53.285 | 22.846 | 32.155 | 1.00 | 45.38 |
| ATOM | 3963 | CA | ILE | A | 518 | 51.915 | 22.378 | 32.232 | 1.00 | 47.13 |
| ATOM | 3964 | C | ILE | A | 518 | 51.552 | 22.224 | 33.670 | 1.00 | 48.18 |
| ATOM | 3965 | O | ILE | A | 518 | 52.152 | 21.424 | 34.374 | 1.00 | 48.20 |
| ATOM | 3966 | CB | ILE | A | 518 | 51.776 | 21.025 | 31.508 | 1.00 | 47.75 |
| ATOM | 3967 | CG1 | ILE | A | 518 | 51.830 | 21.240 | 30.010 | 1.00 | 48.10 |
| ATOM | 3968 | CG2 | ILE | A | 518 | 50.454 | 20.360 | 31.843 | 1.00 | 47.35 |
| ATOM | 3969 | CD1 | ILE | A | 518 | 51.486 | 19.994 | 29.206 | 1.00 | 49.90 |
| ATOM | 3970 | N | LEU | A | 519 | 50.574 | 22.983 | 34.133 | 1.00 | 49.54 |
| ATOM | 3971 | CA | LEU | A | 519 | 50.291 | 22.939 | 35.565 | 1.00 | 50.67 |
| ATOM | 3972 | C | LEU | A | 519 | 49.224 | 21.931 | 35.914 | 1.00 | 51.47 |
| ATOM | 3973 | O | LEU | A | 519 | 49.546 | 20.850 | 36.438 | 1.00 | 52.15 |
| ATOM | 3974 | CB | LEU | A | 519 | 50.001 | 24.321 | 36.129 | 1.00 | 50.73 |
| ATOM | 3975 | CG | LEU | A | 519 | 51.301 | 24.933 | 36.671 | 1.00 | 51.17 |
| ATOM | 3976 | CD1 | LEU | A | 519 | 51.149 | 26.363 | 37.118 | 1.00 | 50.67 |
| ATOM | 3977 | CD2 | LEU | A | 519 | 51.828 | 24.102 | 37.830 | 1.00 | 52.15 |
| ATOM | 3978 | N | ASN | A | 520 | 47.967 | 22.235 | 35.651 | 1.00 | 51.84 |
| ATOM | 3979 | CA | ASN | A | 520 | 46.957 | 21.211 | 35.875 | 1.00 | 52.12 |
| ATOM | 3980 | C | ASN | A | 520 | 46.840 | 20.538 | 34.517 | 1.00 | 51.91 |
| ATOM | 3981 | O | ASN | A | 520 | 47.726 | 19.752 | 34.154 | 1.00 | 53.00 |
| ATOM | 3982 | CB | ASN | A | 520 | 45.681 | 21.818 | 36.416 | 1.00 | 52.46 |
| ATOM | 3983 | CG | ASN | A | 520 | 45.876 | 22.412 | 37.808 | 1.00 | 53.98 |
| ATOM | 3984 | OD1 | ASN | A | 520 | 46.298 | 21.731 | 38.728 | 1.00 | 56.71 |
| ATOM | 3985 | ND2 | ASN | A | 520 | 45.598 | 23.699 | 37.952 | 1.00 | 57.42 |
| ATOM | 3986 | N | GLU | A | 521 | 45.812 | 20.818 | 33.738 | 1.00 | 50.80 |
| ATOM | 3987 | CA | GLU | A | 521 | 45.839 | 20.331 | 32.357 | 1.00 | 50.22 |
| ATOM | 3988 | C | GLU | A | 521 | 45.979 | 21.585 | 31.510 | 1.00 | 48.04 |
| ATOM | 3989 | O | GLU | A | 521 | 45.363 | 21.690 | 30.466 | 1.00 | 48.73 |
| ATOM | 3990 | CB | GLU | A | 521 | 44.530 | 19.654 | 31.917 | 1.00 | 51.10 |
| ATOM | 3991 | CG | GLU | A | 521 | 44.052 | 18.403 | 32.650 | 1.00 | 53.30 |
| ATOM | 3992 | CD | GLU | A | 521 | 42.565 | 18.109 | 32.365 | 1.00 | 55.46 |
| ATOM | 3993 | OE1 | GLU | A | 521 | 42.090 | 18.267 | 31.189 | 1.00 | 55.06 |
| ATOM | 3994 | OE2 | GLU | A | 521 | 41.856 | 17.724 | 33.318 | 1.00 | 56.66 |
| ATOM | 3995 | N | THR | A | 522 | 46.779 | 22.541 | 31.948 | 1.00 | 45.52 |
| ATOM | 3996 | CA | THR | A | 522 | 46.809 | 23.828 | 31.272 | 1.00 | 43.32 |
| ATOM | 3997 | C | THR | A | 522 | 48.208 | 24.414 | 31.061 | 1.00 | 41.37 |
| ATOM | 3998 | O | THR | A | 522 | 49.030 | 24.473 | 31.973 | 1.00 | 39.46 |
| ATOM | 3999 | CB | THR | A | 522 | 45.958 | 24.811 | 32.089 | 1.00 | 43.52 |
| ATOM | 4000 | OG1 | THR | A | 522 | 44.579 | 24.375 | 32.098 | 1.00 | 44.31 |
| ATOM | 4001 | CG2 | THR | A | 522 | 45.954 | 26.204 | 31.432 | 1.00 | 43.41 |
| ATOM | 4002 | N | LYS | A | 523 | 48.439 | 24.893 | 29.851 | 1.00 | 39.61 |
| ATOM | 4003 | CA | LYS | A | 523 | 49.709 | 25.512 | 29.493 | 1.00 | 38.88 |
| ATOM | 4004 | C | LYS | A | 523 | 49.904 | 26.925 | 30.083 | 1.00 | 36.51 |
| ATOM | 4005 | O | LYS | A | 523 | 49.067 | 27.784 | 29.929 | 1.00 | 34.48 |
| ATOM | 4006 | CB | LYS | A | 523 | 49.799 | 25.620 | 27.971 | 1.00 | 39.44 |
| ATOM | 4007 | CG | LYS | A | 523 | 49.762 | 24.274 | 27.245 | 1.00 | 43.67 |
| ATOM | 4008 | CD | LYS | A | 523 | 50.100 | 24.496 | 25.745 | 1.00 | 47.88 |
| ATOM | 4009 | CE | LYS | A | 523 | 49.565 | 23.373 | 24.868 | 1.00 | 51.58 |
| ATOM | 4010 | NZ | LYS | A | 523 | 49.673 | 23.651 | 23.387 | 1.00 | 53.42 |
| ATOM | 4011 | N | PHE | A | 524 | 51.032 | 27.145 | 30.746 | 1.00 | 34.85 |
| ATOM | 4012 | CA | PHE | A | 524 | 51.373 | 28.458 | 31.256 | 1.00 | 33.50 |
| ATOM | 4013 | C | PHE | A | 524 | 52.734 | 28.794 | 30.726 | 1.00 | 33.08 |
| ATOM | 4014 | O | PHE | A | 524 | 53.660 | 28.011 | 30.865 | 1.00 | 34.18 |
| ATOM | 4015 | CB | PHE | A | 524 | 51.317 | 28.465 | 32.750 | 1.00 | 32.45 |
| ATOM | 4016 | CG | PHE | A | 524 | 49.951 | 28.366 | 33.268 | 1.00 | 30.56 |
| ATOM | 4017 | CD1 | PHE | A | 524 | 49.126 | 29.469 | 33.244 | 1.00 | 29.53 |
| ATOM | 4018 | CD2 | PHE | A | 524 | 49.474 | 27.187 | 33.785 | 1.00 | 30.79 |
| ATOM | 4019 | CE1 | PHE | A | 524 | 47.881 | 29.403 | 33.723 | 1.00 | 30.52 |
| ATOM | 4020 | CE2 | PHE | A | 524 | 48.182 | 27.100 | 34.289 | 1.00 | 29.32 |
| ATOM | 4021 | CZ | PHE | A | 524 | 47.384 | 28.194 | 34.262 | 1.00 | 30.30 |
| ATOM | 4022 | N | TRP | A | 525 | 52.846 | 29.957 | 30.104 | 1.00 | 31.67 |
| ATOM | 4023 | CA | TRP | A | 525 | 54.031 | 30.328 | 29.376 | 1.00 | 30.71 |
| ATOM | 4024 | C | TRP | A | 525 | 55.013 | 31.079 | 30.236 | 1.00 | 31.46 |
| ATOM | 4025 | O | TRP | A | 525 | 54.614 | 31.813 | 31.157 | 1.00 | 30.78 |
| ATOM | 4026 | CB | TRP | A | 525 | 53.606 | 31.198 | 28.194 | 1.00 | 30.95 |
| ATOM | 4027 | CG | TRP | A | 525 | 52.901 | 30.439 | 27.136 | 1.00 | 28.92 |
| ATOM | 4028 | CD1 | TRP | A | 525 | 51.590 | 30.062 | 27.110 | 1.00 | 32.14 |
| ATOM | 4029 | CD2 | TRP | A | 525 | 53.497 | 29.891 | 25.966 | 1.00 | 32.39 |
| ATOM | 4030 | NE1 | TRP | A | 525 | 51.330 | 29.328 | 25.975 | 1.00 | 33.71 |
| ATOM | 4031 | CE2 | TRP | A | 525 | 52.485 | 29.216 | 25.248 | 1.00 | 32.14 |
| ATOM | 4032 | CE3 | TRP | A | 525 | 54.787 | 29.912 | 25.439 | 1.00 | 31.16 |
| ATOM | 4033 | CZ2 | TRP | A | 525 | 52.726 | 28.570 | 24.045 | 1.00 | 32.27 |
| ATOM | 4034 | CZ3 | TRP | A | 525 | 55.016 | 29.271 | 24.217 | 1.00 | 35.24 |
| ATOM | 4035 | CH2 | TRP | A | 525 | 53.984 | 28.624 | 23.541 | 1.00 | 33.52 |
| ATOM | 4036 | N | TYR | A | 526 | 56.301 | 30.894 | 29.944 | 1.00 | 31.49 |
| ATOM | 4037 | CA | TYR | A | 526 | 57.342 | 31.643 | 30.601 | 1.00 | 31.28 |
| ATOM | 4038 | C | TYR | A | 526 | 58.430 | 31.992 | 29.634 | 1.00 | 31.22 |
| ATOM | 4039 | O | TYR | A | 526 | 58.557 | 31.404 | 28.561 | 1.00 | 29.20 |
| ATOM | 4040 | CB | TYR | A | 526 | 57.960 | 30.847 | 31.721 | 1.00 | 31.72 |
| ATOM | 4041 | CG | TYR | A | 526 | 58.767 | 29.648 | 31.266 | 1.00 | 33.50 |
| ATOM | 4042 | CD1 | TYR | A | 526 | 58.139 | 28.468 | 30.883 | 1.00 | 36.96 |
| ATOM | 4043 | CD2 | TYR | A | 526 | 60.134 | 29.677 | 31.275 | 1.00 | 36.22 |
| ATOM | 4044 | CE1 | TYR | A | 526 | 58.855 | 27.372 | 30.484 | 1.00 | 36.32 |
| ATOM | 4045 | CE2 | TYR | A | 526 | 60.882 | 28.576 | 30.878 | 1.00 | 38.01 |
| ATOM | 4046 | CZ | TYR | A | 526 | 60.232 | 27.431 | 30.478 | 1.00 | 38.10 |
| ATOM | 4047 | OH | TYR | A | 526 | 60.965 | 26.341 | 30.083 | 1.00 | 37.14 |
| ATOM | 4048 | N | GLN | A | 527 | 59.238 | 32.967 | 30.046 | 1.00 | 31.39 |
| ATOM | 4049 | CA | GLN | A | 527 | 60.408 | 33.335 | 29.294 | 1.00 | 30.10 |
| ATOM | 4050 | C | GLN | A | 527 | 61.579 | 33.417 | 30.234 | 1.00 | 30.59 |
| ATOM | 4051 | O | GLN | A | 527 | 61.444 | 33.834 | 31.391 | 1.00 | 29.47 |
| ATOM | 4052 | CB | GLN | A | 527 | 60.223 | 34.657 | 28.566 | 1.00 | 30.41 |
| ATOM | 4053 | CG | GLN | A | 527 | 59.922 | 35.884 | 29.404 | 1.00 | 29.04 |
| ATOM | 4054 | CD | GLN | A | 527 | 60.067 | 37.144 | 28.614 | 1.00 | 26.06 |
| ATOM | 4055 | OE1 | GLN | A | 527 | 59.464 | 37.288 | 27.526 | 1.00 | 26.65 |
| ATOM | 4056 | NE2 | GLN | A | 527 | 60.888 | 38.066 | 29.113 | 1.00 | 20.18 |
| ATOM | 4057 | N | MET | A | 528 | 62.735 | 33.028 | 29.730 | 1.00 | 30.18 |
| ATOM | 4058 | CA | MET | A | 528 | 63.929 | 33.136 | 30.485 | 1.00 | 31.26 |
| ATOM | 4059 | C | MET | A | 528 | 64.915 | 33.930 | 29.690 | 1.00 | 31.92 |
| ATOM | 4060 | O | MET | A | 528 | 65.091 | 33.695 | 28.494 | 1.00 | 31.39 |
| ATOM | 4061 | CB | MET | A | 528 | 64.508 | 31.751 | 30.758 | 1.00 | 32.07 |
| ATOM | 4062 | CG | MET | A | 528 | 63.987 | 31.170 | 31.968 | 1.00 | 32.27 |
| ATOM | 4063 | SD | MET | A | 528 | 64.481 | 29.594 | 32.377 | 1.00 | 36.87 |
| ATOM | 4064 | CE | MET | A | 528 | 65.854 | 29.504 | 31.652 | 1.00 | 40.66 |
| ATOM | 4065 | N | ILE | A | 529 | 65.569 | 34.873 | 30.349 | 1.00 | 31.96 |
| ATOM | 4066 | CA | ILE | A | 529 | 66.678 | 35.526 | 29.725 | 1.00 | 32.44 |
| ATOM | 4067 | C | ILE | A | 529 | 67.992 | 34.929 | 30.256 | 1.00 | 33.22 |
| ATOM | 4068 | O | ILE | A | 529 | 68.279 | 34.971 | 31.458 | 1.00 | 32.16 |
| ATOM | 4069 | CB | ILE | A | 529 | 66.513 | 37.018 | 29.784 | 1.00 | 33.33 |
| ATOM | 4070 | CG1 | ILE | A | 529 | 65.543 | 37.355 | 28.620 | 1.00 | 34.42 |
| ATOM | 4071 | CG2 | ILE | A | 529 | 67.834 | 37.722 | 29.522 | 1.00 | 32.58 |
| ATOM | 4072 | CD1 | ILE | A | 529 | 64.917 | 38.586 | 28.695 | 1.00 | 35.57 |
| ATOM | 4073 | N | LEU | A | 530 | 68.765 | 34.342 | 29.333 | 1.00 | 33.52 |
| ATOM | 4074 | CA | LEU | A | 530 | 69.910 | 33.529 | 29.689 | 1.00 | 34.80 |
| ATOM | 4075 | C | LEU | A | 530 | 71.246 | 34.171 | 29.402 | 1.00 | 35.01 |
| ATOM | 4076 | O | LEU | A | 530 | 71.452 | 34.784 | 28.361 | 1.00 | 34.02 |
| ATOM | 4077 | CB | LEU | A | 530 | 69.862 | 32.182 | 28.957 | 1.00 | 35.02 |
| ATOM | 4078 | CG | LEU | A | 530 | 68.562 | 31.387 | 29.099 | 1.00 | 36.00 |
| ATOM | 4079 | CD1 | LEU | A | 530 | 68.568 | 30.215 | 28.183 | 1.00 | 38.87 |
| ATOM | 4080 | CD2 | LEU | A | 530 | 68.343 | 30.900 | 30.488 | 1.00 | 37.15 |
| ATOM | 4081 | N | PRO | A | 531 | 72.153 | 34.019 | 30.358 | 1.00 | 35.65 |
| ATOM | 4082 | CA | PRO | A | 531 | 73.519 | 34.505 | 30.216 | 1.00 | 36.34 |
| ATOM | 4083 | C | PRO | A | 531 | 74.156 | 33.893 | 28.989 | 1.00 | 37.00 |
| ATOM | 4084 | O | PRO | A | 531 | 73.874 | 32.737 | 28.657 | 1.00 | 36.54 |
| ATOM | 4085 | CB | PRO | A | 531 | 74.219 | 33.979 | 31.475 | 1.00 | 36.87 |
| ATOM | 4086 | CG | PRO | A | 531 | 73.102 | 33.815 | 32.504 | 1.00 | 36.79 |
| ATOM | 4087 | CD | PRO | A | 531 | 71.903 | 33.399 | 31.665 | 1.00 | 35.99 |
| ATOM | 4088 | N | PRO | A | 532 | 75.065 | 34.615 | 28.370 | 1.00 | 38.89 |
| ATOM | 4089 | CA | PRO | A | 532 | 75.736 | 34.112 | 27.172 | 1.00 | 40.94 |
| ATOM | 4090 | C | PRO | A | 532 | 76.540 | 32.899 | 27.578 | 1.00 | 42.87 |
| ATOM | 4091 | O | PRO | A | 532 | 76.969 | 32.844 | 28.726 | 1.00 | 42.62 |
| ATOM | 4092 | CB | PRO | A | 532 | 76.687 | 35.249 | 26.799 | 1.00 | 40.71 |
| ATOM | 4093 | CG | PRO | A | 532 | 76.937 | 35.960 | 28.098 | 1.00 | 40.62 |
| ATOM | 4094 | CD | PRO | A | 532 | 75.653 | 35.878 | 28.850 | 1.00 | 39.14 |
| ATOM | 4095 | N | HIS | A | 533 | 76.762 | 31.956 | 26.671 | 1.00 | 45.30 |
| ATOM | 4096 | CA | HIS | A | 533 | 77.589 | 30.799 | 27.004 | 1.00 | 46.60 |
| ATOM | 4097 | C | HIS | A | 533 | 76.869 | 30.036 | 28.093 | 1.00 | 47.82 |
| ATOM | 4098 | O | HIS | A | 533 | 77.483 | 29.434 | 28.973 | 1.00 | 48.86 |
| ATOM | 4099 | CB | HIS | A | 533 | 78.962 | 31.264 | 27.483 | 1.00 | 46.56 |
| ATOM | 4100 | CG | HIS | A | 533 | 79.694 | 32.095 | 26.477 | 1.00 | 49.41 |
| ATOM | 4101 | ND1 | HIS | A | 533 | 80.744 | 32.924 | 26.812 | 1.00 | 52.76 |
| ATOM | 4102 | CD2 | HIS | A | 533 | 79.527 | 32.227 | 25.138 | 1.00 | 50.29 |
| ATOM | 4103 | CE1 | HIS | A | 533 | 81.196 | 33.523 | 25.723 | 1.00 | 51.60 |
| ATOM | 4104 | NE2 | HIS | A | 533 | 80.479 | 33.111 | 24.695 | 1.00 | 51.07 |
| ATOM | 4105 | N | PHE | A | 534 | 75.548 | 30.097 | 28.045 | 1.00 | 48.63 |
| ATOM | 4106 | CA | PHE | A | 534 | 74.730 | 29.426 | 29.019 | 1.00 | 48.96 |
| ATOM | 4107 | C | PHE | A | 534 | 75.164 | 27.993 | 29.055 | 1.00 | 50.42 |
| ATOM | 4108 | O | PHE | A | 534 | 75.581 | 27.452 | 28.046 | 1.00 | 50.40 |
| ATOM | 4109 | CB | PHE | A | 534 | 73.264 | 29.510 | 28.629 | 1.00 | 48.57 |
| ATOM | 4110 | CG | PHE | A | 534 | 72.362 | 28.767 | 29.551 | 1.00 | 47.33 |
| ATOM | 4111 | CD1 | PHE | A | 534 | 72.208 | 29.175 | 30.859 | 1.00 | 46.58 |
| ATOM | 4112 | CD2 | PHE | A | 534 | 71.681 | 27.654 | 29.117 | 1.00 | 47.50 |
| ATOM | 4113 | CE1 | PHE | A | 534 | 71.388 | 28.498 | 31.708 | 1.00 | 46.08 |
| ATOM | 4114 | CE2 | PHE | A | 534 | 70.848 | 26.969 | 29.965 | 1.00 | 47.28 |
| ATOM | 4115 | CZ | PHE | A | 534 | 70.701 | 27.391 | 31.263 | 1.00 | 46.52 |
| ATOM | 4116 | N | ASP | A | 535 | 75.064 | 27.363 | 30.219 | 1.00 | 51.90 |
| ATOM | 4117 | CA | ASP | A | 535 | 75.465 | 25.969 | 30.334 | 1.00 | 52.77 |
| ATOM | 4118 | C | ASP | A | 535 | 74.546 | 25.238 | 31.320 | 1.00 | 52.89 |
| ATOM | 4119 | O | ASP | A | 535 | 74.634 | 25.411 | 32.545 | 1.00 | 52.37 |
| ATOM | 4120 | CB | ASP | A | 535 | 76.945 | 25.925 | 30.728 | 1.00 | 53.18 |
| ATOM | 4121 | CG | ASP | A | 535 | 77.425 | 24.531 | 31.095 | 1.00 | 54.56 |
| ATOM | 4122 | OD1 | ASP | A | 535 | 76.631 | 23.564 | 31.033 | 1.00 | 54.82 |
| ATOM | 4123 | OD2 | ASP | A | 535 | 78.594 | 24.330 | 31.494 | 1.00 | 55.84 |
| ATOM | 4124 | N | LYS | A | 536 | 73.669 | 24.415 | 30.762 | 1.00 | 53.04 |
| ATOM | 4125 | CA | LYS | A | 536 | 72.635 | 23.742 | 31.535 | 1.00 | 53.87 |
| ATOM | 4126 | C | LYS | A | 536 | 73.202 | 22.771 | 32.549 | 1.00 | 53.98 |
| ATOM | 4127 | O | LYS | A | 536 | 72.456 | 22.064 | 33.237 | 1.00 | 53.62 |
| ATOM | 4128 | CB | LYS | A | 536 | 71.661 | 23.022 | 30.605 | 1.00 | 54.22 |
| ATOM | 4129 | CG | LYS | A | 536 | 72.271 | 21.887 | 29.778 | 1.00 | 57.03 |
| ATOM | 4130 | CD | LYS | A | 536 | 71.204 | 21.191 | 28.873 | 1.00 | 60.19 |
| ATOM | 4131 | CE | LYS | A | 536 | 71.872 | 20.349 | 27.753 | 1.00 | 62.09 |
| ATOM | 4132 | NZ | LYS | A | 536 | 70.912 | 19.553 | 26.913 | 1.00 | 61.07 |
| ATOM | 4133 | N | SER | A | 537 | 74.526 | 22.740 | 32.628 | 1.00 | 54.14 |
| ATOM | 4134 | CA | SER | A | 537 | 75.216 | 21.897 | 33.572 | 1.00 | 54.42 |
| ATOM | 4135 | C | SER | A | 537 | 75.529 | 22.684 | 34.848 | 1.00 | 53.89 |
| ATOM | 4136 | O | SER | A | 537 | 75.640 | 22.090 | 35.910 | 1.00 | 54.06 |
| ATOM | 4137 | CB | SER | A | 537 | 76.512 | 21.351 | 32.943 | 1.00 | 54.73 |
| ATOM | 4138 | OG | SER | A | 537 | 77.546 | 22.337 | 32.961 | 1.00 | 55.90 |
| ATOM | 4139 | N | LYS | A | 538 | 75.674 | 24.009 | 34.745 | 1.00 | 53.11 |
| ATOM | 4140 | CA | LYS | A | 538 | 75.971 | 24.840 | 35.911 | 1.00 | 52.24 |
| ATOM | 4141 | C | LYS | A | 538 | 74.693 | 25.331 | 36.593 | 1.00 | 50.44 |
| ATOM | 4142 | O | LYS | A | 538 | 73.594 | 25.159 | 36.087 | 1.00 | 50.66 |
| ATOM | 4143 | CB | LYS | A | 538 | 76.875 | 26.004 | 35.526 | 1.00 | 53.03 |
| ATOM | 4144 | CG | LYS | A | 538 | 78.368 | 25.591 | 35.291 | 1.00 | 56.29 |
| ATOM | 4145 | CD | LYS | A | 538 | 79.214 | 25.570 | 36.598 | 1.00 | 59.62 |
| ATOM | 4146 | CE | LYS | A | 538 | 80.632 | 24.978 | 36.375 | 1.00 | 61.60 |
| ATOM | 4147 | NZ | LYS | A | 538 | 81.579 | 25.308 | 37.502 | 1.00 | 62.13 |
| ATOM | 4148 | N | LYS | A | 539 | 74.817 | 25.915 | 37.768 | 1.00 | 48.21 |
| ATOM | 4149 | CA | LYS | A | 539 | 73.621 | 26.344 | 38.484 | 1.00 | 46.43 |
| ATOM | 4150 | C | LYS | A | 539 | 73.671 | 27.826 | 38.569 | 1.00 | 43.84 |
| ATOM | 4151 | O | LYS | A | 539 | 74.626 | 28.379 | 39.085 | 1.00 | 44.47 |
| ATOM | 4152 | CB | LYS | A | 539 | 73.561 | 25.731 | 39.872 | 1.00 | 46.45 |
| ATOM | 4153 | CG | LYS | A | 539 | 73.409 | 24.226 | 39.859 | 1.00 | 48.24 |
| ATOM | 4154 | CD | LYS | A | 539 | 72.651 | 23.732 | 41.073 | 1.00 | 51.70 |
| ATOM | 4155 | CE | LYS | A | 539 | 72.770 | 22.224 | 41.266 | 1.00 | 54.22 |
| ATOM | 4156 | NZ | LYS | A | 539 | 72.715 | 21.841 | 42.745 | 1.00 | 55.81 |
| ATOM | 4157 | N | TYR | A | 540 | 72.667 | 28.481 | 38.019 | 1.00 | 41.27 |
| ATOM | 4158 | CA | TYR | A | 540 | 72.652 | 29.927 | 38.024 | 1.00 | 38.92 |
| ATOM | 4159 | C | TYR | A | 540 | 71.631 | 30.452 | 39.038 | 1.00 | 37.60 |
| ATOM | 4160 | O | TYR | A | 540 | 70.668 | 29.765 | 39.412 | 1.00 | 37.20 |
| ATOM | 4161 | CB | TYR | A | 540 | 72.319 | 30.444 | 36.636 | 1.00 | 38.62 |
| ATOM | 4162 | CG | TYR | A | 540 | 73.256 | 30.008 | 35.541 | 1.00 | 36.88 |
| ATOM | 4163 | CD1 | TYR | A | 540 | 73.229 | 28.715 | 35.043 | 1.00 | 38.33 |
| ATOM | 4164 | CD2 | TYR | A | 540 | 74.154 | 30.904 | 34.981 | 1.00 | 36.48 |
| ATOM | 4165 | CE1 | TYR | A | 540 | 74.084 | 28.324 | 34.034 | 1.00 | 37.41 |
| ATOM | 4166 | CE2 | TYR | A | 540 | 75.007 | 30.526 | 33.998 | 1.00 | 36.38 |
| ATOM | 4167 | CZ | TYR | A | 540 | 74.972 | 29.240 | 33.519 | 1.00 | 38.66 |
| ATOM | 4168 | OH | TYR | A | 540 | 75.824 | 28.891 | 32.493 | 1.00 | 42.11 |
| ATOM | 4169 | N | PRO | A | 541 | 71.882 | 31.646 | 39.535 | 1.00 | 35.14 |
| ATOM | 4170 | CA | PRO | A | 541 | 70.912 | 32.313 | 40.364 | 1.00 | 34.83 |
| ATOM | 4171 | C | PRO | A | 541 | 69.819 | 32.812 | 39.436 | 1.00 | 34.12 |
| ATOM | 4172 | O | PRO | A | 541 | 70.087 | 33.081 | 38.244 | 1.00 | 34.78 |
| ATOM | 4173 | CB | PRO | A | 541 | 71.685 | 33.482 | 40.946 | 1.00 | 34.50 |
| ATOM | 4174 | CG | PRO | A | 541 | 72.834 | 33.650 | 40.130 | 1.00 | 34.44 |
| ATOM | 4175 | CD | PRO | A | 541 | 73.110 | 32.418 | 39.401 | 1.00 | 35.37 |
| ATOM | 4176 | N | LEU | A | 542 | 68.623 | 32.968 | 39.980 | 1.00 | 32.64 |
| ATOM | 4177 | CA | LEU | A | 542 | 67.495 | 33.359 | 39.177 | 1.00 | 32.09 |
| ATOM | 4178 | C | LEU | A | 542 | 66.729 | 34.491 | 39.834 | 1.00 | 31.18 |
| ATOM | 4179 | O | LEU | A | 542 | 66.435 | 34.432 | 41.008 | 1.00 | 31.28 |
| ATOM | 4180 | CB | LEU | A | 542 | 66.613 | 32.139 | 38.958 | 1.00 | 32.37 |
| ATOM | 4181 | CG | LEU | A | 542 | 65.383 | 32.342 | 38.059 | 1.00 | 32.55 |
| ATOM | 4182 | CD1 | LEU | A | 542 | 65.322 | 31.290 | 36.999 | 1.00 | 31.31 |
| ATOM | 4183 | CD2 | LEU | A | 542 | 64.158 | 32.280 | 38.873 | 1.00 | 33.56 |
| ATOM | 4184 | N | LEU | A | 543 | 66.479 | 35.536 | 39.052 | 1.00 | 30.31 |
| ATOM | 4185 | CA | LEU | A | 543 | 65.644 | 36.658 | 39.420 | 1.00 | 28.73 |
| ATOM | 4186 | C | LEU | A | 543 | 64.297 | 36.502 | 38.690 | 1.00 | 28.32 |
| ATOM | 4187 | O | LEU | A | 543 | 64.246 | 36.430 | 37.459 | 1.00 | 24.84 |
| ATOM | 4188 | CB | LEU | A | 543 | 66.273 | 37.943 | 38.944 | 1.00 | 28.34 |
| ATOM | 4189 | CG | LEU | A | 543 | 66.028 | 39.272 | 39.658 | 1.00 | 28.31 |
| ATOM | 4190 | CD1 | LEU | A | 543 | 66.019 | 40.462 | 38.697 | 1.00 | 25.54 |
| ATOM | 4191 | CD2 | LEU | A | 543 | 64.824 | 39.281 | 40.610 | 1.00 | 27.96 |
| ATOM | 4192 | N | LEU | A | 544 | 63.208 | 36.433 | 39.447 | 1.00 | 28.18 |
| ATOM | 4193 | CA | LEU | A | 544 | 61.890 | 36.357 | 38.829 | 1.00 | 28.88 |
| ATOM | 4194 | C | LEU | A | 544 | 61.387 | 37.757 | 38.789 | 1.00 | 28.80 |
| ATOM | 4195 | O | LEU | A | 544 | 61.193 | 38.406 | 39.845 | 1.00 | 29.06 |
| ATOM | 4196 | CB | LEU | A | 544 | 60.930 | 35.574 | 39.667 | 1.00 | 29.67 |
| ATOM | 4197 | CG | LEU | A | 544 | 60.008 | 34.455 | 39.230 | 1.00 | 30.70 |
| ATOM | 4198 | CD1 | LEU | A | 544 | 58.839 | 34.547 | 40.212 | 1.00 | 31.74 |
| ATOM | 4199 | CD2 | LEU | A | 544 | 59.531 | 34.492 | 37.868 | 1.00 | 33.99 |
| ATOM | 4200 | N | ASP | A | 545 | 61.145 | 38.192 | 37.565 | 1.00 | 27.80 |
| ATOM | 4201 | CA | ASP | A | 545 | 60.688 | 39.513 | 37.227 | 1.00 | 27.84 |
| ATOM | 4202 | C | ASP | A | 545 | 59.175 | 39.409 | 37.011 | 1.00 | 27.55 |
| ATOM | 4203 | O | ASP | A | 545 | 58.687 | 38.741 | 36.077 | 1.00 | 27.66 |
| ATOM | 4204 | CB | ASP | A | 545 | 61.472 | 39.939 | 35.972 | 1.00 | 28.12 |
| ATOM | 4205 | CG | ASP | A | 545 | 61.014 | 41.229 | 35.379 | 1.00 | 29.19 |
| ATOM | 4206 | OD1 | ASP | A | 545 | 60.215 | 41.964 | 36.003 | 1.00 | 34.08 |
| ATOM | 4207 | OD2 | ASP | A | 545 | 61.391 | 41.583 | 34.254 | 1.00 | 28.60 |
| ATOM | 4208 | N | VAL | A | 546 | 58.422 | 40.032 | 37.895 | 1.00 | 26.54 |
| ATOM | 4209 | CA | VAL | A | 546 | 56.983 | 39.913 | 37.860 | 1.00 | 26.42 |
| ATOM | 4210 | C | VAL | A | 546 | 56.196 | 41.198 | 37.625 | 1.00 | 25.04 |
| ATOM | 4211 | O | VAL | A | 546 | 56.581 | 42.293 | 38.035 | 1.00 | 24.61 |
| ATOM | 4212 | CB | VAL | A | 546 | 56.498 | 39.350 | 39.181 | 1.00 | 27.68 |
| ATOM | 4213 | CG1 | VAL | A | 546 | 54.993 | 39.144 | 39.152 | 1.00 | 27.49 |
| ATOM | 4214 | CG2 | VAL | A | 546 | 57.196 | 38.014 | 39.445 | 1.00 | 29.91 |
| ATOM | 4215 | N | TYR | A | 547 | 55.064 | 41.041 | 36.959 | 1.00 | 23.69 |
| ATOM | 4216 | CA | TYR | A | 547 | 54.117 | 42.110 | 36.842 | 1.00 | 22.57 |
| ATOM | 4217 | C | TYR | A | 547 | 52.846 | 41.436 | 37.281 | 1.00 | 22.98 |
| ATOM | 4218 | O | TYR | A | 547 | 52.406 | 41.613 | 38.424 | 1.00 | 23.27 |
| ATOM | 4219 | CB | TYR | A | 547 | 54.052 | 42.699 | 35.441 | 1.00 | 23.31 |
| ATOM | 4220 | CG | TYR | A | 547 | 53.074 | 43.801 | 35.462 | 1.00 | 25.16 |
| ATOM | 4221 | CD1 | TYR | A | 547 | 53.351 | 44.984 | 36.153 | 1.00 | 25.12 |
| ATOM | 4222 | CD2 | TYR | A | 547 | 51.763 | 43.607 | 34.939 | 1.00 | 24.22 |
| ATOM | 4223 | CE1 | TYR | A | 547 | 52.371 | 46.016 | 36.257 | 1.00 | 25.27 |
| ATOM | 4224 | CE2 | TYR | A | 547 | 50.826 | 44.615 | 35.008 | 1.00 | 24.61 |
| ATOM | 4225 | CZ | TYR | A | 547 | 51.115 | 45.787 | 35.667 | 1.00 | 23.64 |
| ATOM | 4226 | OH | TYR | A | 547 | 50.117 | 46.657 | 35.763 | 1.00 | 24.55 |
| ATOM | 4227 | N | ALA | A | 548 | 52.265 | 40.636 | 36.385 | 1.00 | 22.11 |
| ATOM | 4228 | CA | ALA | A | 548 | 51.173 | 39.768 | 36.700 | 1.00 | 20.92 |
| ATOM | 4229 | C | ALA | A | 548 | 49.835 | 40.380 | 37.101 | 1.00 | 21.97 |
| ATOM | 4230 | O | ALA | A | 548 | 49.047 | 39.706 | 37.671 | 1.00 | 21.06 |
| ATOM | 4231 | CB | ALA | A | 548 | 51.612 | 38.766 | 37.761 | 1.00 | 21.37 |
| ATOM | 4232 | N | GLY | A | 549 | 49.547 | 41.623 | 36.780 | 1.00 | 22.48 |
| ATOM | 4233 | CA | GLY | A | 549 | 48.216 | 42.097 | 37.014 | 1.00 | 23.62 |
| ATOM | 4234 | C | GLY | A | 549 | 47.287 | 41.502 | 35.950 | 1.00 | 24.09 |
| ATOM | 4235 | O | GLY | A | 549 | 47.764 | 40.960 | 34.964 | 1.00 | 23.68 |
| ATOM | 4236 | N | PRO | A | 550 | 45.979 | 41.659 | 36.160 | 1.00 | 24.27 |
| ATOM | 4237 | CA | PRO | A | 550 | 44.918 | 41.212 | 35.243 | 1.00 | 25.13 |
| ATOM | 4238 | C | PRO | A | 550 | 45.109 | 41.623 | 33.781 | 1.00 | 24.88 |
| ATOM | 4239 | O | PRO | A | 550 | 45.328 | 42.799 | 33.463 | 1.00 | 22.84 |
| ATOM | 4240 | CB | PRO | A | 550 | 43.678 | 41.928 | 35.775 | 1.00 | 25.99 |
| ATOM | 4241 | CG | PRO | A | 550 | 43.973 | 42.352 | 37.159 | 1.00 | 25.28 |
| ATOM | 4242 | CD | PRO | A | 550 | 45.442 | 42.315 | 37.358 | 1.00 | 24.62 |
| ATOM | 4243 | N | CYS | A | 551 | 45.029 | 40.637 | 32.898 | 1.00 | 26.44 |
| ATOM | 4244 | CA | CYS | A | 551 | 45.190 | 40.856 | 31.450 | 1.00 | 27.76 |
| ATOM | 4245 | C | CYS | A | 551 | 46.656 | 41.022 | 30.984 | 1.00 | 28.00 |
| ATOM | 4246 | O | CYS | A | 551 | 46.934 | 41.154 | 29.798 | 1.00 | 29.90 |
| ATOM | 4247 | CB | CYS | A | 551 | 44.397 | 42.083 | 31.040 | 1.00 | 27.76 |
| ATOM | 4248 | SG | CYS | A | 551 | 42.666 | 41.973 | 31.557 | 1.00 | 33.83 |
| ATOM | 4249 | N | SER | A | 552 | 47.621 | 41.003 | 31.890 | 1.00 | 27.10 |
| ATOM | 4250 | CA | SER | A | 552 | 48.960 | 41.299 | 31.451 | 1.00 | 26.44 |
| ATOM | 4251 | C | SER | A | 552 | 49.660 | 40.071 | 30.870 | 1.00 | 25.68 |
| ATOM | 4252 | O | SER | A | 552 | 49.178 | 38.944 | 30.987 | 1.00 | 24.80 |
| ATOM | 4253 | CB | SER | A | 552 | 49.758 | 41.845 | 32.618 | 1.00 | 26.57 |
| ATOM | 4254 | OG | SER | A | 552 | 49.863 | 40.817 | 33.568 | 1.00 | 29.75 |
| ATOM | 4255 | N | GLN | A | 553 | 50.792 | 40.323 | 30.226 | 1.00 | 25.00 |
| ATOM | 4256 | CA | GLN | A | 553 | 51.598 | 39.286 | 29.634 | 1.00 | 25.59 |
| ATOM | 4257 | C | GLN | A | 553 | 53.039 | 39.648 | 29.727 | 1.00 | 25.83 |
| ATOM | 4258 | O | GLN | A | 553 | 53.472 | 40.647 | 29.162 | 1.00 | 25.04 |
| ATOM | 4259 | CB | GLN | A | 553 | 51.244 | 39.143 | 28.159 | 1.00 | 25.82 |
| ATOM | 4260 | CG | GLN | A | 553 | 52.067 | 38.059 | 27.375 | 1.00 | 26.19 |
| ATOM | 4261 | CD | GLN | A | 553 | 51.497 | 37.831 | 25.969 | 1.00 | 24.66 |
| ATOM | 4262 | OE1 | GLN | A | 553 | 51.699 | 38.647 | 25.100 | 1.00 | 28.01 |
| ATOM | 4263 | NE2 | GLN | A | 553 | 50.793 | 36.736 | 25.767 | 1.00 | 22.88 |
| ATOM | 4264 | N | LYS | A | 554 | 53.795 | 38.812 | 30.418 | 1.00 | 27.54 |
| ATOM | 4265 | CA | LYS | A | 554 | 55.215 | 39.028 | 30.609 | 1.00 | 28.83 |
| ATOM | 4266 | C | LYS | A | 554 | 56.100 | 38.043 | 29.838 | 1.00 | 29.25 |
| ATOM | 4267 | O | LYS | A | 554 | 57.315 | 38.218 | 29.801 | 1.00 | 29.54 |
| ATOM | 4268 | CB | LYS | A | 554 | 55.530 | 38.858 | 32.095 | 1.00 | 30.04 |
| ATOM | 4269 | CG | LYS | A | 554 | 55.528 | 40.130 | 32.889 | 1.00 | 32.17 |
| ATOM | 4270 | CD | LYS | A | 554 | 56.755 | 40.982 | 32.524 | 1.00 | 32.20 |
| ATOM | 4271 | CE | LYS | A | 554 | 57.468 | 41.547 | 33.734 | 1.00 | 29.61 |
| ATOM | 4272 | NZ | LYS | A | 554 | 58.540 | 42.527 | 33.279 | 1.00 | 26.97 |
| ATOM | 4273 | N | ALA | A | 555 | 55.527 | 36.971 | 29.303 | 1.00 | 29.98 |
| ATOM | 4274 | CA | ALA | A | 555 | 56.279 | 36.058 | 28.419 | 1.00 | 29.87 |
| ATOM | 4275 | C | ALA | A | 555 | 55.928 | 36.357 | 26.981 | 1.00 | 28.92 |
| ATOM | 4276 | O | ALA | A | 555 | 54.829 | 36.135 | 26.591 | 1.00 | 28.84 |
| ATOM | 4277 | CB | ALA | A | 555 | 55.908 | 34.628 | 28.717 | 1.00 | 30.59 |
| ATOM | 4278 | N | ASP | A | 556 | 56.845 | 36.848 | 26.175 | 1.00 | 29.29 |
| ATOM | 4279 | CA | ASP | A | 556 | 56.496 | 37.164 | 24.775 | 1.00 | 28.88 |
| ATOM | 4280 | C | ASP | A | 556 | 57.712 | 37.131 | 23.885 | 1.00 | 28.69 |
| ATOM | 4281 | O | ASP | A | 556 | 58.808 | 36.879 | 24.369 | 1.00 | 29.50 |
| ATOM | 4282 | CB | ASP | A | 556 | 55.814 | 38.516 | 24.700 | 1.00 | 28.24 |
| ATOM | 4283 | CG | ASP | A | 556 | 56.709 | 39.646 | 25.081 | 1.00 | 29.28 |
| ATOM | 4284 | OD1 | ASP | A | 556 | 57.958 | 39.515 | 24.984 | 1.00 | 28.75 |
| ATOM | 4285 | OD2 | ASP | A | 556 | 56.227 | 40.762 | 25.408 | 1.00 | 33.08 |
| ATOM | 4286 | N | THR | A | 557 | 57.547 | 37.437 | 22.605 | 1.00 | 27.97 |
| ATOM | 4287 | CA | THR | A | 557 | 58.664 | 37.344 | 21.675 | 1.00 | 27.37 |
| ATOM | 4288 | C | THR | A | 557 | 59.265 | 38.698 | 21.354 | 1.00 | 27.44 |
| ATOM | 4289 | O | THR | A | 557 | 59.921 | 38.854 | 20.326 | 1.00 | 27.29 |
| ATOM | 4290 | CB | THR | A | 557 | 58.224 | 36.741 | 20.355 | 1.00 | 27.18 |
| ATOM | 4291 | OG1 | THR | A | 557 | 57.098 | 37.466 | 19.876 | 1.00 | 25.91 |
| ATOM | 4292 | CG2 | THR | A | 557 | 57.702 | 35.365 | 20.505 | 1.00 | 26.17 |
| ATOM | 4293 | N | VAL | A | 558 | 59.072 | 39.666 | 22.229 | 1.00 | 27.15 |
| ATOM | 4294 | CA | VAL | A | 558 | 59.628 | 40.993 | 22.018 | 1.00 | 26.81 |
| ATOM | 4295 | C | VAL | A | 558 | 61.123 | 41.105 | 22.381 | 1.00 | 27.19 |
| ATOM | 4296 | O | VAL | A | 558 | 61.608 | 40.551 | 23.373 | 1.00 | 26.77 |
| ATOM | 4297 | CB | VAL | A | 558 | 58.806 | 42.038 | 22.782 | 1.00 | 27.50 |
| ATOM | 4298 | CG1 | VAL | A | 558 | 59.372 | 43.437 | 22.654 | 1.00 | 27.07 |
| ATOM | 4299 | CG2 | VAL | A | 558 | 57.362 | 42.018 | 22.308 | 1.00 | 26.92 |
| ATOM | 4300 | N | PHE | A | 559 | 61.851 | 41.822 | 21.517 | 1.00 | 27.05 |
| ATOM | 4301 | CA | PHE | A | 559 | 63.237 | 42.117 | 21.722 | 1.00 | 26.81 |
| ATOM | 4302 | C | PHE | A | 559 | 63.436 | 43.293 | 22.649 | 1.00 | 26.85 |
| ATOM | 4303 | O | PHE | A | 559 | 63.027 | 44.379 | 22.344 | 1.00 | 26.95 |
| ATOM | 4304 | CB | PHE | A | 559 | 63.920 | 42.482 | 20.405 | 1.00 | 26.92 |
| ATOM | 4305 | CG | PHE | A | 559 | 65.371 | 42.745 | 20.567 | 1.00 | 28.33 |
| ATOM | 4306 | CD1 | PHE | A | 559 | 66.240 | 41.694 | 20.770 | 1.00 | 31.89 |
| ATOM | 4307 | CD2 | PHE | A | 559 | 65.855 | 44.047 | 20.679 | 1.00 | 30.73 |
| ATOM | 4308 | CE1 | PHE | A | 559 | 67.630 | 41.947 | 20.978 | 1.00 | 34.04 |
| ATOM | 4309 | CE2 | PHE | A | 559 | 67.233 | 44.304 | 20.905 | 1.00 | 31.47 |
| ATOM | 4310 | CZ | PHE | A | 559 | 68.107 | 43.258 | 21.044 | 1.00 | 31.74 |
| ATOM | 4311 | N | ARG | A | 560 | 64.173 | 43.118 | 23.729 | 1.00 | 27.22 |
| ATOM | 4312 | CA | ARG | A | 560 | 64.360 | 44.244 | 24.644 | 1.00 | 28.08 |
| ATOM | 4313 | C | ARG | A | 560 | 65.819 | 44.392 | 25.069 | 1.00 | 28.04 |
| ATOM | 4314 | O | ARG | A | 560 | 66.505 | 43.423 | 25.237 | 1.00 | 27.21 |
| ATOM | 4315 | CB | ARG | A | 560 | 63.498 | 44.072 | 25.894 | 1.00 | 28.98 |
| ATOM | 4316 | CG | ARG | A | 560 | 61.936 | 44.178 | 25.701 | 1.00 | 29.93 |
| ATOM | 4317 | CD | ARG | A | 560 | 61.099 | 44.022 | 27.041 | 1.00 | 31.30 |
| ATOM | 4318 | NE | ARG | A | 560 | 59.699 | 44.108 | 26.716 | 1.00 | 30.89 |
| ATOM | 4319 | CZ | ARG | A | 560 | 58.919 | 43.085 | 26.413 | 1.00 | 31.97 |
| ATOM | 4320 | NH1 | ARG | A | 560 | 59.348 | 41.814 | 26.465 | 1.00 | 30.16 |
| ATOM | 4321 | NH2 | ARG | A | 560 | 57.679 | 43.349 | 26.051 | 1.00 | 30.62 |
| ATOM | 4322 | N | LEU | A | 561 | 66.271 | 45.632 | 25.207 | 1.00 | 28.54 |
| ATOM | 4323 | CA | LEU | A | 561 | 67.570 | 45.931 | 25.806 | 1.00 | 28.49 |
| ATOM | 4324 | C | LEU | A | 561 | 67.253 | 46.660 | 27.118 | 1.00 | 27.77 |
| ATOM | 4325 | O | LEU | A | 561 | 66.930 | 47.867 | 27.145 | 1.00 | 26.56 |
| ATOM | 4326 | CB | LEU | A | 561 | 68.402 | 46.835 | 24.924 | 1.00 | 28.27 |
| ATOM | 4327 | CG | LEU | A | 561 | 68.919 | 46.215 | 23.622 | 1.00 | 30.62 |
| ATOM | 4328 | CD1 | LEU | A | 561 | 69.466 | 47.274 | 22.684 | 1.00 | 30.40 |
| ATOM | 4329 | CD2 | LEU | A | 561 | 69.999 | 45.183 | 23.927 | 1.00 | 29.88 |
| ATOM | 4330 | N | ASN | A | 562 | 67.359 | 45.935 | 28.212 | 1.00 | 26.75 |
| ATOM | 4331 | CA | ASN | A | 562 | 66.937 | 46.508 | 29.482 | 1.00 | 26.54 |
| ATOM | 4332 | C | ASN | A | 562 | 67.811 | 46.000 | 30.646 | 1.00 | 25.75 |
| ATOM | 4333 | O | ASN | A | 562 | 68.890 | 45.429 | 30.409 | 1.00 | 24.65 |
| ATOM | 4334 | CB | ASN | A | 562 | 65.450 | 46.218 | 29.655 | 1.00 | 26.84 |
| ATOM | 4335 | CG | ASN | A | 562 | 65.165 | 44.728 | 29.713 | 1.00 | 27.06 |
| ATOM | 4336 | OD1 | ASN | A | 562 | 66.089 | 43.920 | 29.842 | 1.00 | 23.61 |
| ATOM | 4337 | ND2 | ASN | A | 562 | 63.884 | 44.361 | 29.627 | 1.00 | 26.04 |
| ATOM | 4338 | N | TRP | A | 563 | 67.379 | 46.230 | 31.884 | 1.00 | 24.68 |
| ATOM | 4339 | CA | TRP | A | 563 | 68.154 | 45.843 | 33.063 | 1.00 | 24.77 |
| ATOM | 4340 | C | TRP | A | 563 | 68.391 | 44.332 | 33.121 | 1.00 | 25.75 |
| ATOM | 4341 | O | TRP | A | 563 | 69.484 | 43.859 | 33.452 | 1.00 | 23.51 |
| ATOM | 4342 | CB | TRP | A | 563 | 67.428 | 46.310 | 34.321 | 1.00 | 24.44 |
| ATOM | 4343 | CG | TRP | A | 563 | 68.183 | 46.093 | 35.607 | 1.00 | 24.88 |
| ATOM | 4344 | CD1 | TRP | A | 563 | 69.474 | 46.449 | 35.897 | 1.00 | 25.62 |
| ATOM | 4345 | CD2 | TRP | A | 563 | 67.652 | 45.528 | 36.800 | 1.00 | 22.46 |
| ATOM | 4346 | NE1 | TRP | A | 563 | 69.770 | 46.121 | 37.202 | 1.00 | 24.87 |
| ATOM | 4347 | CE2 | TRP | A | 563 | 68.665 | 45.554 | 37.773 | 1.00 | 23.96 |
| ATOM | 4348 | CE3 | TRP | A | 563 | 66.390 | 45.038 | 37.159 | 1.00 | 23.58 |
| ATOM | 4349 | CZ2 | TRP | A | 563 | 68.475 | 45.061 | 39.064 | 1.00 | 25.00 |
| ATOM | 4350 | CZ3 | TRP | A | 563 | 66.213 | 44.541 | 38.417 | 1.00 | 22.02 |
| ATOM | 4351 | CH2 | TRP | A | 563 | 67.245 | 44.559 | 39.362 | 1.00 | 21.67 |
| ATOM | 4352 | N | ALA | A | 564 | 67.343 | 43.567 | 32.846 | 1.00 | 26.67 |
| ATOM | 4353 | CA | ALA | A | 564 | 67.496 | 42.122 | 32.730 | 1.00 | 28.06 |
| ATOM | 4354 | C | ALA | A | 564 | 68.614 | 41.771 | 31.714 | 1.00 | 28.72 |
| ATOM | 4355 | O | ALA | A | 564 | 69.345 | 40.782 | 31.882 | 1.00 | 28.25 |
| ATOM | 4356 | CB | ALA | A | 564 | 66.158 | 41.476 | 32.294 | 1.00 | 28.63 |
| ATOM | 4357 | N | THR | A | 565 | 68.770 | 42.592 | 30.685 | 1.00 | 29.69 |
| ATOM | 4358 | CA | THR | A | 565 | 69.811 | 42.339 | 29.683 | 1.00 | 30.16 |
| ATOM | 4359 | C | THR | A | 565 | 71.167 | 42.353 | 30.409 | 1.00 | 30.65 |
| ATOM | 4360 | O | THR | A | 565 | 71.984 | 41.421 | 30.272 | 1.00 | 30.39 |
| ATOM | 4361 | CB | THR | A | 565 | 69.786 | 43.408 | 28.561 | 1.00 | 30.79 |
| ATOM | 4362 | OG1 | THR | A | 565 | 68.577 | 43.308 | 27.825 | 1.00 | 29.29 |
| ATOM | 4363 | CG2 | THR | A | 565 | 70.853 | 43.135 | 27.490 | 1.00 | 33.12 |
| ATOM | 4364 | N | TYR | A | 566 | 71.387 | 43.401 | 31.190 | 1.00 | 30.55 |
| ATOM | 4365 | CA | TYR | A | 566 | 72.617 | 43.541 | 31.963 | 1.00 | 30.73 |
| ATOM | 4366 | C | TYR | A | 566 | 72.833 | 42.434 | 32.971 | 1.00 | 30.63 |
| ATOM | 4367 | O | TYR | A | 566 | 73.909 | 41.869 | 33.032 | 1.00 | 30.91 |
| ATOM | 4368 | CB | TYR | A | 566 | 72.681 | 44.910 | 32.660 | 1.00 | 30.89 |
| ATOM | 4369 | CG | TYR | A | 566 | 73.394 | 44.888 | 34.001 | 1.00 | 31.09 |
| ATOM | 4370 | CD1 | TYR | A | 566 | 74.779 | 44.595 | 34.106 | 1.00 | 31.58 |
| ATOM | 4371 | CD2 | TYR | A | 566 | 72.685 | 45.143 | 35.166 | 1.00 | 31.02 |
| ATOM | 4372 | CE1 | TYR | A | 566 | 75.412 | 44.568 | 35.345 | 1.00 | 28.78 |
| ATOM | 4373 | CE2 | TYR | A | 566 | 73.286 | 45.138 | 36.393 | 1.00 | 27.99 |
| ATOM | 4374 | CZ | TYR | A | 566 | 74.642 | 44.849 | 36.493 | 1.00 | 30.72 |
| ATOM | 4375 | OH | TYR | A | 566 | 75.193 | 44.877 | 37.735 | 1.00 | 25.46 |
| ATOM | 4376 | N | LEU | A | 567 | 71.825 | 42.130 | 33.775 | 1.00 | 30.20 |
| ATOM | 4377 | CA | LEU | A | 567 | 71.937 | 41.081 | 34.782 | 1.00 | 29.05 |
| ATOM | 4378 | C | LEU | A | 567 | 72.382 | 39.749 | 34.170 | 1.00 | 29.55 |
| ATOM | 4379 | O | LEU | A | 567 | 73.157 | 38.957 | 34.758 | 1.00 | 28.19 |
| ATOM | 4380 | CB | LEU | A | 567 | 70.582 | 40.904 | 35.473 | 1.00 | 28.13 |
| ATOM | 4381 | CG | LEU | A | 567 | 70.233 | 42.068 | 36.424 | 1.00 | 27.20 |
| ATOM | 4382 | CD1 | LEU | A | 567 | 68.905 | 41.852 | 37.092 | 1.00 | 25.37 |
| ATOM | 4383 | CD2 | LEU | A | 567 | 71.305 | 42.331 | 37.526 | 1.00 | 27.82 |
| ATOM | 4384 | N | ALA | A | 568 | 71.823 | 39.465 | 33.004 | 1.00 | 30.10 |
| ATOM | 4385 | CA | ALA | A | 568 | 72.149 | 38.232 | 32.334 | 1.00 | 29.91 |
| ATOM | 4386 | C | ALA | A | 568 | 73.547 | 38.312 | 31.721 | 1.00 | 29.69 |
| ATOM | 4387 | O | ALA | A | 568 | 74.336 | 37.417 | 31.925 | 1.00 | 29.40 |
| ATOM | 4388 | CB | ALA | A | 568 | 71.083 | 37.882 | 31.288 | 1.00 | 29.91 |
| ATOM | 4389 | N | SER | A | 569 | 73.888 | 39.405 | 31.053 | 1.00 | 30.36 |
| ATOM | 4390 | CA | SER | A | 569 | 75.130 | 39.433 | 30.266 | 1.00 | 30.98 |
| ATOM | 4391 | C | SER | A | 569 | 76.370 | 39.652 | 31.096 | 1.00 | 31.60 |
| ATOM | 4392 | O | SER | A | 569 | 77.354 | 39.000 | 30.873 | 1.00 | 31.24 |
| ATOM | 4393 | CB | SER | A | 569 | 75.062 | 40.484 | 29.180 | 1.00 | 31.49 |
| ATOM | 4394 | OG | SER | A | 569 | 76.343 | 40.827 | 28.698 | 1.00 | 29.58 |
| ATOM | 4395 | N | THR | A | 570 | 76.308 | 40.573 | 32.054 | 1.00 | 31.57 |
| ATOM | 4396 | CA | THR | A | 570 | 77.428 | 40.830 | 32.929 | 1.00 | 31.14 |
| ATOM | 4397 | C | THR | A | 570 | 77.448 | 40.013 | 34.226 | 1.00 | 31.26 |
| ATOM | 4398 | O | THR | A | 570 | 78.502 | 39.507 | 34.588 | 1.00 | 31.27 |
| ATOM | 4399 | CB | THR | A | 570 | 77.469 | 42.292 | 33.327 | 1.00 | 31.50 |
| ATOM | 4400 | OG1 | THR | A | 570 | 77.579 | 43.136 | 32.170 | 1.00 | 28.62 |
| ATOM | 4401 | CG2 | THR | A | 570 | 78.754 | 42.568 | 34.151 | 1.00 | 33.70 |
| ATOM | 4402 | N | GLU | A | 571 | 76.311 | 39.873 | 34.926 | 1.00 | 30.73 |
| ATOM | 4403 | CA | GLU | A | 571 | 76.299 | 39.189 | 36.216 | 1.00 | 29.94 |
| ATOM | 4404 | C | GLU | A | 571 | 75.955 | 37.733 | 36.172 | 1.00 | 30.81 |
| ATOM | 4405 | O | GLU | A | 571 | 76.038 | 37.043 | 37.190 | 1.00 | 31.01 |
| ATOM | 4406 | CB | GLU | A | 571 | 75.343 | 39.876 | 37.187 | 1.00 | 29.77 |
| ATOM | 4407 | CG | GLU | A | 571 | 75.566 | 41.362 | 37.397 | 1.00 | 30.28 |
| ATOM | 4408 | CD | GLU | A | 571 | 76.980 | 41.725 | 37.807 | 1.00 | 31.41 |
| ATOM | 4409 | OE1 | GLU | A | 571 | 77.675 | 40.823 | 38.285 | 1.00 | 30.55 |
| ATOM | 4410 | OE2 | GLU | A | 571 | 77.371 | 42.920 | 37.677 | 1.00 | 30.19 |
| ATOM | 4411 | N | ASN | A | 572 | 75.536 | 37.230 | 35.013 | 1.00 | 31.60 |
| ATOM | 4412 | CA | ASN | A | 572 | 75.242 | 35.815 | 34.903 | 1.00 | 30.95 |
| ATOM | 4413 | C | ASN | A | 572 | 74.082 | 35.378 | 35.749 | 1.00 | 29.28 |
| ATOM | 4414 | O | ASN | A | 572 | 74.063 | 34.293 | 36.321 | 1.00 | 28.59 |
| ATOM | 4415 | CB | ASN | A | 572 | 76.479 | 35.003 | 35.238 | 1.00 | 32.51 |
| ATOM | 4416 | CG | ASN | A | 572 | 77.530 | 35.092 | 34.159 | 1.00 | 36.25 |
| ATOM | 4417 | OD1 | ASN | A | 572 | 77.243 | 34.856 | 32.986 | 1.00 | 41.33 |
| ATOM | 4418 | ND2 | ASN | A | 572 | 78.753 | 35.470 | 34.543 | 1.00 | 40.87 |
| ATOM | 4419 | N | ILE | A | 573 | 73.068 | 36.223 | 35.792 | 1.00 | 28.23 |
| ATOM | 4420 | CA | ILE | A | 573 | 71.842 | 35.885 | 36.463 | 1.00 | 27.13 |
| ATOM | 4421 | C | ILE | A | 573 | 70.791 | 35.515 | 35.405 | 1.00 | 27.67 |
| ATOM | 4422 | O | ILE | A | 573 | 70.650 | 36.199 | 34.423 | 1.00 | 26.39 |
| ATOM | 4423 | CB | ILE | A | 573 | 71.390 | 37.108 | 37.262 | 1.00 | 27.08 |
| ATOM | 4424 | CG1 | ILE | A | 573 | 72.377 | 37.423 | 38.395 | 1.00 | 27.09 |
| ATOM | 4425 | CG2 | ILE | A | 573 | 70.028 | 36.899 | 37.835 | 1.00 | 27.15 |
| ATOM | 4426 | CD1 | ILE | A | 573 | 72.341 | 38.882 | 38.866 | 1.00 | 25.44 |
| ATOM | 4427 | N | ILE | A | 574 | 70.039 | 34.442 | 35.617 | 1.00 | 28.39 |
| ATOM | 4428 | CA | ILE | A | 574 | 68.933 | 34.131 | 34.744 | 1.00 | 29.05 |
| ATOM | 4429 | C | ILE | A | 574 | 67.765 | 35.024 | 35.172 | 1.00 | 29.34 |
| ATOM | 4430 | O | ILE | A | 574 | 67.456 | 35.119 | 36.363 | 1.00 | 29.44 |
| ATOM | 4431 | CB | ILE | A | 574 | 68.522 | 32.663 | 34.893 | 1.00 | 28.69 |
| ATOM | 4432 | CG1 | ILE | A | 574 | 69.543 | 31.741 | 34.226 | 1.00 | 31.22 |
| ATOM | 4433 | CG2 | ILE | A | 574 | 67.198 | 32.456 | 34.291 | 1.00 | 28.04 |
| ATOM | 4434 | CD1 | ILE | A | 574 | 69.232 | 30.289 | 34.468 | 1.00 | 32.74 |
| ATOM | 4435 | N | VAL | A | 575 | 67.112 | 35.667 | 34.216 | 1.00 | 29.53 |
| ATOM | 4436 | CA | VAL | A | 575 | 65.965 | 36.483 | 34.548 | 1.00 | 29.76 |
| ATOM | 4437 | C | VAL | A | 575 | 64.707 | 35.903 | 33.932 | 1.00 | 29.95 |
| ATOM | 4438 | O | VAL | A | 575 | 64.543 | 35.898 | 32.711 | 1.00 | 29.63 |
| ATOM | 4439 | CB | VAL | A | 575 | 66.160 | 37.860 | 34.098 | 1.00 | 29.91 |
| ATOM | 4440 | CG1 | VAL | A | 575 | 64.879 | 38.687 | 34.402 | 1.00 | 31.25 |
| ATOM | 4441 | CG2 | VAL | A | 575 | 67.391 | 38.441 | 34.821 | 1.00 | 29.45 |
| ATOM | 4442 | N | ALA | A | 576 | 63.813 | 35.409 | 34.780 | 1.00 | 28.39 |
| ATOM | 4443 | CA | ALA | A | 576 | 62.617 | 34.777 | 34.288 | 1.00 | 28.29 |
| ATOM | 4444 | C | ALA | A | 576 | 61.318 | 35.498 | 34.631 | 1.00 | 28.18 |
| ATOM | 4445 | O | ALA | A | 576 | 61.207 | 36.161 | 35.676 | 1.00 | 27.41 |
| ATOM | 4446 | CB | ALA | A | 576 | 62.559 | 33.389 | 34.810 | 1.00 | 29.28 |
| ATOM | 4447 | N | SER | A | 577 | 60.340 | 35.324 | 33.745 | 1.00 | 27.14 |
| ATOM | 4448 | CA | SER | A | 577 | 58.982 | 35.797 | 33.957 | 1.00 | 27.54 |
| ATOM | 4449 | C | SER | A | 577 | 57.993 | 34.732 | 33.539 | 1.00 | 27.49 |
| ATOM | 4450 | O | SER | A | 577 | 58.283 | 33.898 | 32.696 | 1.00 | 28.59 |
| ATOM | 4451 | CB | SER | A | 577 | 58.714 | 37.075 | 33.240 | 1.00 | 27.19 |
| ATOM | 4452 | OG | SER | A | 577 | 59.805 | 37.939 | 33.396 | 1.00 | 29.78 |
| ATOM | 4453 | N | PHE | A | 578 | 56.832 | 34.788 | 34.162 | 1.00 | 27.77 |
| ATOM | 4454 | CA | PHE | A | 578 | 55.822 | 33.771 | 34.094 | 1.00 | 28.34 |
| ATOM | 4455 | C | PHE | A | 578 | 54.423 | 34.359 | 34.048 | 1.00 | 28.40 |
| ATOM | 4456 | O | PHE | A | 578 | 54.080 | 35.247 | 34.838 | 1.00 | 27.15 |
| ATOM | 4457 | CB | PHE | A | 578 | 55.908 | 32.933 | 35.361 | 1.00 | 28.57 |
| ATOM | 4458 | CG | PHE | A | 578 | 54.948 | 31.793 | 35.386 | 1.00 | 29.13 |
| ATOM | 4459 | CD1 | PHE | A | 578 | 55.138 | 30.712 | 34.551 | 1.00 | 33.36 |
| ATOM | 4460 | CD2 | PHE | A | 578 | 53.870 | 31.787 | 36.237 | 1.00 | 27.73 |
| ATOM | 4461 | CE1 | PHE | A | 578 | 54.263 | 29.647 | 34.562 | 1.00 | 31.73 |
| ATOM | 4462 | CE2 | PHE | A | 578 | 53.023 | 30.741 | 36.270 | 1.00 | 28.38 |
| ATOM | 4463 | CZ | PHE | A | 578 | 53.208 | 29.666 | 35.431 | 1.00 | 29.75 |
| ATOM | 4464 | N | ASP | A | 579 | 53.628 | 33.838 | 33.125 | 1.00 | 28.25 |
| ATOM | 4465 | CA | ASP | A | 579 | 52.275 | 34.249 | 32.922 | 1.00 | 28.24 |
| ATOM | 4466 | C | ASP | A | 579 | 51.388 | 33.162 | 33.532 | 1.00 | 28.42 |
| ATOM | 4467 | O | ASP | A | 579 | 51.185 | 32.113 | 32.916 | 1.00 | 27.61 |
| ATOM | 4468 | CB | ASP | A | 579 | 52.006 | 34.452 | 31.419 | 1.00 | 28.54 |
| ATOM | 4469 | CG | ASP | A | 579 | 52.595 | 35.790 | 30.869 | 1.00 | 30.75 |
| ATOM | 4470 | OD1 | ASP | A | 579 | 52.820 | 36.746 | 31.647 | 1.00 | 30.82 |
| ATOM | 4471 | OD2 | ASP | A | 579 | 52.830 | 36.006 | 29.642 | 1.00 | 34.72 |
| ATOM | 4472 | N | GLY | A | 580 | 50.914 | 33.399 | 34.771 | 1.00 | 28.17 |
| ATOM | 4473 | CA | GLY | A | 580 | 50.018 | 32.489 | 35.469 | 1.00 | 27.52 |
| ATOM | 4474 | C | GLY | A | 580 | 48.557 | 32.909 | 35.376 | 1.00 | 27.45 |
| ATOM | 4475 | O | GLY | A | 580 | 48.182 | 33.736 | 34.523 | 1.00 | 26.73 |
| ATOM | 4476 | N | ARG | A | 581 | 47.710 | 32.384 | 36.267 | 1.00 | 27.25 |
| ATOM | 4477 | CA | ARG | A | 581 | 46.301 | 32.769 | 36.224 | 1.00 | 27.30 |
| ATOM | 4478 | C | ARG | A | 581 | 46.152 | 34.269 | 36.324 | 1.00 | 27.40 |
| ATOM | 4479 | O | ARG | A | 581 | 46.910 | 34.949 | 37.008 | 1.00 | 27.28 |
| ATOM | 4480 | CB | ARG | A | 581 | 45.456 | 32.081 | 37.291 | 1.00 | 27.42 |
| ATOM | 4481 | CG | ARG | A | 581 | 45.027 | 30.726 | 36.875 | 1.00 | 28.39 |
| ATOM | 4482 | CD | ARG | A | 581 | 44.672 | 29.758 | 37.961 | 1.00 | 28.82 |
| ATOM | 4483 | NE | ARG | A | 581 | 45.723 | 29.536 | 38.944 | 1.00 | 30.44 |
| ATOM | 4484 | CZ | ARG | A | 581 | 45.496 | 28.949 | 40.118 | 1.00 | 33.24 |
| ATOM | 4485 | NH1 | ARG | A | 581 | 44.255 | 28.618 | 40.427 | 1.00 | 33.68 |
| ATOM | 4486 | NH2 | ARG | A | 581 | 46.484 | 28.702 | 40.991 | 1.00 | 34.06 |
| ATOM | 4487 | N | GLY | A | 582 | 45.169 | 34.771 | 35.599 | 1.00 | 28.05 |
| ATOM | 4488 | CA | GLY | A | 582 | 44.868 | 36.189 | 35.565 | 1.00 | 28.06 |
| ATOM | 4489 | C | GLY | A | 582 | 45.539 | 36.827 | 34.385 | 1.00 | 27.42 |
| ATOM | 4490 | O | GLY | A | 582 | 45.218 | 37.945 | 34.012 | 1.00 | 28.10 |
| ATOM | 4491 | N | SER | A | 583 | 46.501 | 36.129 | 33.811 | 1.00 | 26.84 |
| ATOM | 4492 | CA | SER | A | 583 | 47.200 | 36.668 | 32.668 | 1.00 | 26.81 |
| ATOM | 4493 | C | SER | A | 583 | 46.268 | 36.722 | 31.399 | 1.00 | 26.26 |
| ATOM | 4494 | O | SER | A | 583 | 45.216 | 36.137 | 31.365 | 1.00 | 25.67 |
| ATOM | 4495 | CB | SER | A | 583 | 48.528 | 35.928 | 32.455 | 1.00 | 26.87 |
| ATOM | 4496 | OG | SER | A | 583 | 48.377 | 34.539 | 32.175 | 1.00 | 28.61 |
| ATOM | 4497 | N | GLY | A | 584 | 46.646 | 37.482 | 30.401 | 1.00 | 26.14 |
| ATOM | 4498 | CA | GLY | A | 584 | 45.786 | 37.695 | 29.276 | 1.00 | 26.40 |
| ATOM | 4499 | C | GLY | A | 584 | 46.104 | 36.962 | 28.016 | 1.00 | 26.15 |
| ATOM | 4500 | O | GLY | A | 584 | 47.047 | 36.179 | 27.922 | 1.00 | 24.92 |
| ATOM | 4501 | N | TYR | A | 585 | 45.233 | 37.221 | 27.057 | 1.00 | 27.27 |
| ATOM | 4502 | CA | TYR | A | 585 | 45.369 | 36.756 | 25.692 | 1.00 | 27.73 |
| ATOM | 4503 | C | TYR | A | 585 | 45.317 | 35.258 | 25.520 | 1.00 | 28.58 |
| ATOM | 4504 | O | TYR | A | 585 | 45.689 | 34.777 | 24.463 | 1.00 | 29.54 |
| ATOM | 4505 | CB | TYR | A | 585 | 46.671 | 37.242 | 25.075 | 1.00 | 28.47 |
| ATOM | 4506 | CG | TYR | A | 585 | 46.852 | 38.704 | 25.203 | 1.00 | 27.03 |
| ATOM | 4507 | CD1 | TYR | A | 585 | 45.971 | 39.574 | 24.589 | 1.00 | 26.76 |
| ATOM | 4508 | CD2 | TYR | A | 585 | 47.837 | 39.221 | 26.028 | 1.00 | 22.71 |
| ATOM | 4509 | CE1 | TYR | A | 585 | 46.085 | 40.922 | 24.731 | 1.00 | 24.58 |
| ATOM | 4510 | CE2 | TYR | A | 585 | 47.969 | 40.585 | 26.195 | 1.00 | 23.53 |
| ATOM | 4511 | CZ | TYR | A | 585 | 47.103 | 41.424 | 25.539 | 1.00 | 25.17 |
| ATOM | 4512 | OH | TYR | A | 585 | 47.229 | 42.752 | 25.675 | 1.00 | 27.86 |
| ATOM | 4513 | N | GLN | A | 586 | 44.822 | 34.533 | 26.509 | 1.00 | 27.92 |
| ATOM | 4514 | CA | GLN | A | 586 | 44.741 | 33.086 | 26.421 | 1.00 | 27.66 |
| ATOM | 4515 | C | GLN | A | 586 | 43.313 | 32.610 | 26.790 | 1.00 | 27.85 |
| ATOM | 4516 | O | GLN | A | 586 | 43.090 | 31.424 | 27.118 | 1.00 | 26.65 |
| ATOM | 4517 | CB | GLN | A | 586 | 45.751 | 32.452 | 27.387 | 1.00 | 27.58 |
| ATOM | 4518 | CG | GLN | A | 586 | 47.215 | 32.842 | 27.212 | 1.00 | 27.04 |
| ATOM | 4519 | CD | GLN | A | 586 | 47.976 | 32.807 | 28.537 | 1.00 | 26.67 |
| ATOM | 4520 | OE1 | GLN | A | 586 | 48.152 | 33.833 | 29.182 | 1.00 | 30.99 |
| ATOM | 4521 | NE2 | GLN | A | 586 | 48.376 | 31.650 | 28.952 | 1.00 | 24.71 |
| ATOM | 4522 | N | GLY | A | 587 | 42.357 | 33.539 | 26.778 | 1.00 | 27.74 |
| ATOM | 4523 | CA | GLY | A | 587 | 40.976 | 33.202 | 27.070 | 1.00 | 27.65 |
| ATOM | 4524 | C | GLY | A | 587 | 40.580 | 33.584 | 28.491 | 1.00 | 27.30 |
| ATOM | 4525 | O | GLY | A | 587 | 41.413 | 33.852 | 29.356 | 1.00 | 26.71 |
| ATOM | 4526 | N | ASP | A | 588 | 39.281 | 33.622 | 28.719 | 1.00 | 28.24 |
| ATOM | 4527 | CA | ASP | A | 588 | 38.717 | 34.063 | 29.985 | 1.00 | 28.72 |
| ATOM | 4528 | C | ASP | A | 588 | 38.889 | 33.097 | 31.115 | 1.00 | 29.23 |
| ATOM | 4529 | O | ASP | A | 588 | 38.938 | 33.493 | 32.288 | 1.00 | 27.88 |
| ATOM | 4530 | CB | ASP | A | 588 | 37.258 | 34.378 | 29.804 | 1.00 | 28.82 |
| ATOM | 4531 | CG | ASP | A | 588 | 37.048 | 35.713 | 29.127 | 1.00 | 30.71 |
| ATOM | 4532 | OD1 | ASP | A | 588 | 38.045 | 36.441 | 28.955 | 1.00 | 32.40 |
| ATOM | 4533 | OD2 | ASP | A | 588 | 35.934 | 36.115 | 28.737 | 1.00 | 33.98 |
| ATOM | 4534 | N | LYS | A | 589 | 39.025 | 31.821 | 30.794 | 1.00 | 30.05 |
| ATOM | 4535 | CA | LYS | A | 589 | 39.165 | 30.856 | 31.863 | 1.00 | 31.24 |
| ATOM | 4536 | C | LYS | A | 589 | 40.391 | 31.239 | 32.688 | 1.00 | 30.44 |
| ATOM | 4537 | O | LYS | A | 589 | 40.379 | 31.182 | 33.918 | 1.00 | 30.91 |
| ATOM | 4538 | CB | LYS | A | 589 | 39.336 | 29.435 | 31.333 | 1.00 | 31.78 |
| ATOM | 4539 | CG | LYS | A | 589 | 39.665 | 28.450 | 32.480 | 1.00 | 36.61 |
| ATOM | 4540 | CD | LYS | A | 589 | 39.570 | 26.997 | 32.065 | 1.00 | 42.76 |
| ATOM | 4541 | CE | LYS | A | 589 | 40.168 | 26.045 | 33.132 | 1.00 | 45.94 |
| ATOM | 4542 | NZ | LYS | A | 589 | 40.138 | 24.612 | 32.636 | 1.00 | 46.76 |
| ATOM | 4543 | N | ILE | A | 590 | 41.473 | 31.583 | 32.008 | 1.00 | 29.49 |
| ATOM | 4544 | CA | ILE | A | 590 | 42.690 | 31.983 | 32.699 | 1.00 | 29.07 |
| ATOM | 4545 | C | ILE | A | 590 | 42.582 | 33.424 | 33.202 | 1.00 | 28.20 |
| ATOM | 4546 | O | ILE | A | 590 | 42.932 | 33.708 | 34.352 | 1.00 | 29.03 |
| ATOM | 4547 | CB | ILE | A | 590 | 43.917 | 31.792 | 31.766 | 1.00 | 29.03 |
| ATOM | 4548 | CG1 | ILE | A | 590 | 44.305 | 30.299 | 31.697 | 1.00 | 29.00 |
| ATOM | 4549 | CG2 | ILE | A | 590 | 45.086 | 32.594 | 32.253 | 1.00 | 29.24 |
| ATOM | 4550 | CD1 | ILE | A | 590 | 45.365 | 29.961 | 30.622 | 1.00 | 27.13 |
| ATOM | 4551 | N | MET | A | 591 | 42.064 | 34.320 | 32.370 | 1.00 | 28.36 |
| ATOM | 4552 | CA | MET | A | 591 | 42.012 | 35.746 | 32.692 | 1.00 | 28.67 |
| ATOM | 4553 | C | MET | A | 591 | 41.074 | 36.094 | 33.837 | 1.00 | 29.62 |
| ATOM | 4554 | O | MET | A | 591 | 41.422 | 36.927 | 34.660 | 1.00 | 30.74 |
| ATOM | 4555 | CB | MET | A | 591 | 41.635 | 36.608 | 31.503 | 1.00 | 28.91 |
| ATOM | 4556 | CG | MET | A | 591 | 41.965 | 38.046 | 31.699 | 1.00 | 29.05 |
| ATOM | 4557 | SD | MET | A | 591 | 41.724 | 39.127 | 30.289 | 1.00 | 33.19 |
| ATOM | 4558 | CE | MET | A | 591 | 39.938 | 39.178 | 30.224 | 1.00 | 33.59 |
| ATOM | 4559 | N | HIS | A | 592 | 39.926 | 35.430 | 33.923 | 1.00 | 29.45 |
| ATOM | 4560 | CA | HIS | A | 592 | 38.938 | 35.742 | 34.924 | 1.00 | 29.13 |
| ATOM | 4561 | C | HIS | A | 592 | 39.151 | 34.959 | 36.190 | 1.00 | 29.85 |
| ATOM | 4562 | O | HIS | A | 592 | 38.400 | 35.120 | 37.141 | 1.00 | 29.65 |
| ATOM | 4563 | CB | HIS | A | 592 | 37.544 | 35.387 | 34.417 | 1.00 | 29.01 |
| ATOM | 4564 | CG | HIS | A | 592 | 37.013 | 36.312 | 33.371 | 1.00 | 28.57 |
| ATOM | 4565 | ND1 | HIS | A | 592 | 37.575 | 37.539 | 33.089 | 1.00 | 29.42 |
| ATOM | 4566 | CD2 | HIS | A | 592 | 35.917 | 36.216 | 32.593 | 1.00 | 27.96 |
| ATOM | 4567 | CE1 | HIS | A | 592 | 36.884 | 38.128 | 32.138 | 1.00 | 27.95 |
| ATOM | 4568 | NE2 | HIS | A | 592 | 35.857 | 37.352 | 31.834 | 1.00 | 29.38 |
| ATOM | 4569 | N | ALA | A | 593 | 40.165 | 34.119 | 36.235 | 1.00 | 29.91 |
| ATOM | 4570 | CA | ALA | A | 593 | 40.334 | 33.249 | 37.411 | 1.00 | 30.74 |
| ATOM | 4571 | C | ALA | A | 593 | 40.597 | 34.076 | 38.670 | 1.00 | 31.49 |
| ATOM | 4572 | O | ALA | A | 593 | 40.406 | 33.618 | 39.800 | 1.00 | 31.13 |
| ATOM | 4573 | CB | ALA | A | 593 | 41.460 | 32.329 | 37.171 | 1.00 | 30.13 |
| ATOM | 4574 | N | ILE | A | 594 | 41.007 | 35.316 | 38.455 | 1.00 | 31.17 |
| ATOM | 4575 | CA | ILE | A | 594 | 41.370 | 36.177 | 39.556 | 1.00 | 31.77 |
| ATOM | 4576 | C | ILE | A | 594 | 40.275 | 37.221 | 39.868 | 1.00 | 30.82 |
| ATOM | 4577 | O | ILE | A | 594 | 40.446 | 38.085 | 40.734 | 1.00 | 30.47 |
| ATOM | 4578 | CB | ILE | A | 594 | 42.747 | 36.727 | 39.206 | 1.00 | 31.82 |
| ATOM | 4579 | CG1 | ILE | A | 594 | 43.681 | 36.422 | 40.307 | 1.00 | 33.46 |
| ATOM | 4580 | CG2 | ILE | A | 594 | 42.739 | 38.166 | 38.748 | 1.00 | 34.20 |
| ATOM | 4581 | CD1 | ILE | A | 594 | 44.217 | 35.074 | 40.182 | 1.00 | 34.34 |
| ATOM | 4582 | N | ASN | A | 595 | 39.133 | 37.079 | 39.192 | 1.00 | 29.75 |
| ATOM | 4583 | CA | ASN | A | 595 | 37.991 | 37.952 | 39.401 | 1.00 | 29.62 |
| ATOM | 4584 | C | ASN | A | 595 | 37.646 | 38.132 | 40.897 | 1.00 | 29.97 |
| ATOM | 4585 | O | ASN | A | 595 | 37.551 | 37.160 | 41.639 | 1.00 | 28.99 |
| ATOM | 4586 | CB | ASN | A | 595 | 36.750 | 37.415 | 38.678 | 1.00 | 29.57 |
| ATOM | 4587 | CG | ASN | A | 595 | 35.624 | 38.427 | 38.684 | 1.00 | 28.63 |
| ATOM | 4588 | OD1 | ASN | A | 595 | 35.857 | 39.582 | 38.438 | 1.00 | 25.10 |
| ATOM | 4589 | ND2 | ASN | A | 595 | 34.417 | 38.002 | 38.992 | 1.00 | 27.23 |
| ATOM | 4590 | N | ARG | A | 596 | 37.467 | 39.373 | 41.327 | 1.00 | 30.19 |
| ATOM | 4591 | CA | ARG | A | 596 | 37.202 | 39.666 | 42.735 | 1.00 | 31.75 |
| ATOM | 4592 | C | ARG | A | 596 | 38.201 | 39.018 | 43.710 | 1.00 | 31.80 |
| ATOM | 4593 | O | ARG | A | 596 | 37.976 | 39.036 | 44.923 | 1.00 | 30.98 |
| ATOM | 4594 | CB | ARG | A | 596 | 35.733 | 39.305 | 43.130 | 1.00 | 32.08 |
| ATOM | 4595 | CG | ARG | A | 596 | 34.696 | 40.340 | 42.630 | 1.00 | 35.72 |
| ATOM | 4596 | CD | ARG | A | 596 | 33.177 | 39.894 | 42.698 | 1.00 | 41.54 |
| ATOM | 4597 | NE | ARG | A | 596 | 32.405 | 40.460 | 43.834 | 1.00 | 42.59 |
| ATOM | 4598 | CZ | ARG | A | 596 | 32.269 | 39.861 | 45.022 | 1.00 | 47.28 |
| ATOM | 4599 | NH1 | ARG | A | 596 | 32.856 | 38.689 | 45.258 | 1.00 | 51.26 |
| ATOM | 4600 | NH2 | ARG | A | 596 | 31.549 | 40.416 | 45.982 | 1.00 | 46.20 |
| ATOM | 4601 | N | ARG | A | 597 | 39.305 | 38.479 | 43.207 | 1.00 | 31.81 |
| ATOM | 4602 | CA | ARG | A | 597 | 40.270 | 37.827 | 44.073 | 1.00 | 32.75 |
| ATOM | 4603 | C | ARG | A | 597 | 41.699 | 38.280 | 43.789 | 1.00 | 31.55 |
| ATOM | 4604 | O | ARG | A | 597 | 42.568 | 37.437 | 43.658 | 1.00 | 30.88 |
| ATOM | 4605 | CB | ARG | A | 597 | 40.298 | 36.325 | 43.808 | 1.00 | 34.17 |
| ATOM | 4606 | CG | ARG | A | 597 | 39.136 | 35.511 | 44.204 | 1.00 | 39.92 |
| ATOM | 4607 | CD | ARG | A | 597 | 39.324 | 34.062 | 43.701 | 1.00 | 47.98 |
| ATOM | 4608 | NE | ARG | A | 597 | 38.700 | 33.034 | 44.551 | 1.00 | 53.43 |
| ATOM | 4609 | CZ | ARG | A | 597 | 39.353 | 32.310 | 45.465 | 1.00 | 57.69 |
| ATOM | 4610 | NH1 | ARG | A | 597 | 40.672 | 32.474 | 45.688 | 1.00 | 57.37 |
| ATOM | 4611 | NH2 | ARG | A | 597 | 38.677 | 31.412 | 46.168 | 1.00 | 59.75 |
| ATOM | 4612 | N | LEU | A | 598 | 41.967 | 39.573 | 43.672 | 1.00 | 31.19 |
| ATOM | 4613 | CA | LEU | A | 598 | 43.335 | 39.988 | 43.380 | 1.00 | 30.25 |
| ATOM | 4614 | C | LEU | A | 598 | 44.165 | 39.573 | 44.553 | 1.00 | 28.97 |
| ATOM | 4615 | O | LEU | A | 598 | 43.700 | 39.597 | 45.687 | 1.00 | 28.81 |
| ATOM | 4616 | CB | LEU | A | 598 | 43.461 | 41.481 | 43.174 | 1.00 | 30.07 |
| ATOM | 4617 | CG | LEU | A | 598 | 42.638 | 42.085 | 42.067 | 1.00 | 30.76 |
| ATOM | 4618 | CD1 | LEU | A | 598 | 42.995 | 43.563 | 41.972 | 1.00 | 29.68 |
| ATOM | 4619 | CD2 | LEU | A | 598 | 42.834 | 41.359 | 40.724 | 1.00 | 31.70 |
| ATOM | 4620 | N | GLY | A | 599 | 45.382 | 39.146 | 44.271 | 1.00 | 28.60 |
| ATOM | 4621 | CA | GLY | A | 599 | 46.314 | 38.760 | 45.309 | 1.00 | 28.03 |
| ATOM | 4622 | C | GLY | A | 599 | 46.217 | 37.315 | 45.727 | 1.00 | 28.30 |
| ATOM | 4623 | O | GLY | A | 599 | 46.696 | 36.919 | 46.802 | 1.00 | 27.10 |
| ATOM | 4624 | N | THR | A | 600 | 45.586 | 36.490 | 44.903 | 1.00 | 27.74 |
| ATOM | 4625 | CA | TER | A | 600 | 45.567 | 35.098 | 45.253 | 1.00 | 27.97 |
| ATOM | 4626 | C | TER | A | 600 | 46.324 | 34.265 | 44.229 | 1.00 | 28.18 |
| ATOM | 4627 | O | TER | A | 600 | 47.555 | 34.112 | 44.330 | 1.00 | 28.50 |
| ATOM | 4628 | CB | THR | A | 600 | 44.148 | 34.586 | 45.464 | 1.00 | 27.65 |
| ATOM | 4629 | OG1 | THR | A | 600 | 43.360 | 34.878 | 44.315 | 1.00 | 26.47 |
| ATOM | 4630 | CG2 | THR | A | 600 | 43.461 | 35.329 | 46.601 | 1.00 | 28.89 |
| ATOM | 4631 | N | PHE | A | 601 | 45.603 | 33.737 | 43.247 | 1.00 | 27.45 |
| ATOM | 4632 | CA | PHE | A | 601 | 46.192 | 32.799 | 42.293 | 1.00 | 28.22 |
| ATOM | 4633 | C | PHE | A | 601 | 47.363 | 33.381 | 41.488 | 1.00 | 27.67 |
| ATOM | 4634 | O | PHE | A | 601 | 48.336 | 32.689 | 41.225 | 1.00 | 29.22 |
| ATOM | 4635 | CB | PHE | A | 601 | 45.132 | 32.249 | 41.343 | 1.00 | 28.13 |
| ATOM | 4636 | CG | PHE | A | 601 | 43.997 | 31.581 | 42.005 | 1.00 | 30.17 |
| ATOM | 4637 | CD1 | PHE | A | 601 | 44.191 | 30.643 | 42.999 | 1.00 | 33.45 |
| ATOM | 4638 | CD2 | PHE | A | 601 | 42.706 | 31.884 | 41.633 | 1.00 | 35.26 |
| ATOM | 4639 | CE1 | PHE | A | 601 | 43.127 | 30.049 | 43.604 | 1.00 | 33.70 |
| ATOM | 4640 | CE2 | PHE | A | 601 | 41.642 | 31.268 | 42.218 | 1.00 | 33.26 |
| ATOM | 4641 | CZ | PHE | A | 601 | 41.852 | 30.355 | 43.202 | 1.00 | 36.32 |
| ATOM | 4642 | N | GLU | A | 602 | 47.310 | 34.654 | 41.137 | 1.00 | 27.82 |
| ATOM | 4643 | CA | GLU | A | 602 | 48.419 | 35.256 | 40.392 | 1.00 | 27.51 |
| ATOM | 4644 | C | GLU | A | 602 | 49.676 | 35.166 | 41.258 | 1.00 | 27.74 |
| ATOM | 4645 | O | GLU | A | 602 | 50.784 | 34.902 | 40.760 | 1.00 | 28.48 |
| ATOM | 4646 | CB | GLU | A | 602 | 48.095 | 36.702 | 39.939 | 1.00 | 26.94 |
| ATOM | 4647 | CG | GLU | A | 602 | 48.289 | 37.798 | 40.972 | 1.00 | 27.51 |
| ATOM | 4648 | CD | GLU | A | 602 | 47.038 | 38.130 | 41.774 | 1.00 | 28.08 |
| ATOM | 4649 | OE1 | GLU | A | 602 | 46.393 | 37.199 | 42.267 | 1.00 | 29.58 |
| ATOM | 4650 | OE2 | GLU | A | 602 | 46.686 | 39.341 | 41.890 | 1.00 | 26.35 |
| ATOM | 4651 | N | VAL | A | 603 | 49.494 | 35.332 | 42.562 | 1.00 | 28.37 |
| ATOM | 4652 | CA | VAL | A | 603 | 50.600 | 35.245 | 43.516 | 1.00 | 28.70 |
| ATOM | 4653 | C | VAL | A | 603 | 51.096 | 33.806 | 43.640 | 1.00 | 29.00 |
| ATOM | 4654 | O | VAL | A | 603 | 52.237 | 33.523 | 43.361 | 1.00 | 30.16 |
| ATOM | 4655 | CB | VAL | A | 603 | 50.156 | 35.787 | 44.906 | 1.00 | 29.48 |
| ATOM | 4656 | CG1 | VAL | A | 603 | 51.251 | 35.695 | 45.939 | 1.00 | 28.54 |
| ATOM | 4657 | CG2 | VAL | A | 603 | 49.650 | 37.230 | 44.762 | 1.00 | 29.58 |
| ATOM | 4658 | N | GLU | A | 604 | 50.238 | 32.897 | 44.061 | 1.00 | 30.18 |
| ATOM | 4659 | CA | GLU | A | 604 | 50.586 | 31.481 | 44.169 | 1.00 | 31.60 |
| ATOM | 4660 | C | GLU | A | 604 | 51.249 | 30.915 | 42.919 | 1.00 | 31.34 |
| ATOM | 4661 | O | GLU | A | 604 | 52.127 | 30.060 | 43.006 | 1.00 | 30.85 |
| ATOM | 4662 | CB | GLU | A | 604 | 49.326 | 30.629 | 44.364 | 1.00 | 32.23 |
| ATOM | 4663 | CG | GLU | A | 604 | 48.700 | 30.585 | 45.733 | 1.00 | 38.37 |
| ATOM | 4664 | CD | GLU | A | 604 | 47.337 | 29.897 | 45.668 | 1.00 | 44.37 |
| ATOM | 4665 | OE1 | GLU | A | 604 | 47.261 | 28.723 | 45.144 | 1.00 | 45.08 |
| ATOM | 4666 | OE2 | GLU | A | 604 | 46.351 | 30.558 | 46.091 | 1.00 | 45.55 |
| ATOM | 4667 | N | ASP | A | 605 | 50.782 | 31.332 | 41.746 | 1.00 | 31.12 |
| ATOM | 4668 | CA | ASP | A | 605 | 51.362 | 30.797 | 40.513 | 1.00 | 30.50 |
| ATOM | 4669 | C | ASP | A | 605 | 52.785 | 31.271 | 40.283 | 1.00 | 30.00 |
| ATOM | 4670 | O | ASP | A | 605 | 53.571 | 30.559 | 39.716 | 1.00 | 30.11 |
| ATOM | 4671 | CB | ASP | A | 605 | 50.468 | 31.123 | 39.334 | 1.00 | 30.77 |
| ATOM | 4672 | CG | ASP | A | 605 | 49.164 | 30.325 | 39.357 | 1.00 | 31.53 |
| ATOM | 4673 | OD1 | ASP | A | 605 | 49.077 | 29.378 | 40.177 | 1.00 | 31.02 |
| ATOM | 4674 | OD2 | ASP | A | 605 | 48.180 | 30.583 | 38.602 | 1.00 | 28.91 |
| ATOM | 4675 | N | GLN | A | 606 | 53.136 | 32.464 | 40.740 | 1.00 | 30.08 |
| ATOM | 4676 | CA | GLN | A | 606 | 54.516 | 32.921 | 40.641 | 1.00 | 29.63 |
| ATOM | 4677 | C | GLN | A | 606 | 55.396 | 32.053 | 41.538 | 1.00 | 30.06 |
| ATOM | 4678 | O | GLN | A | 606 | 56.483 | 31.648 | 41.155 | 1.00 | 29.24 |
| ATOM | 4679 | CB | GLN | A | 606 | 54.645 | 34.403 | 41.028 | 1.00 | 29.22 |
| ATOM | 4680 | CG | GLN | A | 606 | 54.028 | 35.364 | 40.051 | 1.00 | 28.78 |
| ATOM | 4681 | CD | GLN | A | 606 | 54.724 | 35.385 | 38.726 | 1.00 | 28.81 |
| ATOM | 4682 | OE1 | GLN | A | 606 | 55.950 | 35.570 | 38.649 | 1.00 | 33.30 |
| ATOM | 4683 | NE2 | GLN | A | 606 | 53.977 | 35.165 | 37.682 | 1.00 | 23.28 |
| ATOM | 4684 | N | ILE | A | 607 | 54.916 | 31.767 | 42.741 | 1.00 | 31.56 |
| ATOM | 4685 | CA | ILE | A | 607 | 55.651 | 30.919 | 43.672 | 1.00 | 32.01 |
| ATOM | 4686 | C | ILE | A | 607 | 55.812 | 29.550 | 43.042 | 1.00 | 33.52 |
| ATOM | 4687 | O | ILE | A | 607 | 56.917 | 28.983 | 43.044 | 1.00 | 33.45 |
| ATOM | 4688 | CB | ILE | A | 607 | 54.896 | 30.790 | 45.016 | 1.00 | 32.41 |
| ATOM | 4689 | CG1 | ILE | A | 607 | 54.992 | 32.075 | 45.834 | 1.00 | 32.46 |
| ATOM | 4690 | CG2 | ILE | A | 607 | 55.463 | 29.677 | 45.842 | 1.00 | 32.05 |
| ATOM | 4691 | CD1 | ILE | A | 607 | 54.085 | 32.086 | 47.054 | 1.00 | 33.73 |
| ATOM | 4692 | N | GLU | A | 608 | 54.734 | 29.021 | 42.460 | 1.00 | 34.16 |
| ATOM | 4693 | CA | GLU | A | 608 | 54.821 | 27.694 | 41.843 | 1.00 | 35.73 |
| ATOM | 4694 | C | GLU | A | 608 | 55.761 | 27.695 | 40.616 | 1.00 | 35.46 |
| ATOM | 4695 | O | GLU | A | 608 | 56.487 | 26.736 | 40.398 | 1.00 | 35.66 |
| ATOM | 4696 | CB | GLU | A | 608 | 53.418 | 27.127 | 41.539 | 1.00 | 36.56 |
| ATOM | 4697 | CG | GLU | A | 608 | 53.359 | 25.710 | 40.955 | 1.00 | 40.03 |
| ATOM | 4698 | CD | GLU | A | 608 | 53.927 | 24.616 | 41.855 | 1.00 | 44.24 |
| ATOM | 4699 | OE1 | GLU | A | 608 | 53.808 | 24.703 | 43.100 | 1.00 | 44.37 |
| ATOM | 4700 | OE2 | GLU | A | 608 | 54.515 | 23.652 | 41.300 | 1.00 | 48.00 |
| ATOM | 4701 | N | ALA | A | 609 | 55.802 | 28.788 | 39.859 | 1.00 | 34.52 |
| ATOM | 4702 | CA | ALA | A | 609 | 56.667 | 28.828 | 38.693 | 1.00 | 34.51 |
| ATOM | 4703 | C | ALA | A | 609 | 58.149 | 28.716 | 39.114 | 1.00 | 34.13 |
| ATOM | 4704 | O | ALA | A | 609 | 58.961 | 28.006 | 38.469 | 1.00 | 31.78 |
| ATOM | 4705 | CB | ALA | A | 609 | 56.437 | 30.088 | 37.884 | 1.00 | 33.49 |
| ATOM | 4706 | N | ALA | A | 610 | 58.479 | 29.447 | 40.171 | 1.00 | 34.05 |
| ATOM | 4707 | CA | ALA | A | 610 | 59.832 | 29.413 | 40.730 | 1.00 | 34.67 |
| ATOM | 4708 | C | ALA | A | 610 | 60.224 | 28.026 | 41.238 | 1.00 | 35.08 |
| ATOM | 4709 | O | ALA | A | 610 | 61.354 | 27.575 | 41.035 | 1.00 | 33.89 |
| ATOM | 4710 | CB | ALA | A | 610 | 59.988 | 30.448 | 41.822 | 1.00 | 34.17 |
| ATOM | 4711 | N | ARG | A | 611 | 59.298 | 27.339 | 41.883 | 1.00 | 36.94 |
| ATOM | 4712 | CA | ARG | A | 611 | 59.574 | 25.963 | 42.292 | 1.00 | 38.63 |
| ATOM | 4713 | C | ARG | A | 611 | 59.873 | 25.104 | 41.068 | 1.00 | 39.64 |
| ATOM | 4714 | O | ARG | A | 611 | 60.775 | 24.276 | 41.108 | 1.00 | 39.08 |
| ATOM | 4715 | CB | ARG | A | 611 | 58.406 | 25.322 | 43.009 | 1.00 | 38.66 |
| ATOM | 4716 | CG | ARG | A | 611 | 58.196 | 25.752 | 44.416 | 1.00 | 40.91 |
| ATOM | 4717 | CD | ARG | A | 611 | 57.304 | 24.810 | 45.195 | 1.00 | 42.36 |
| ATOM | 4718 | NE | ARG | A | 611 | 56.252 | 25.580 | 45.850 | 1.00 | 47.02 |
| ATOM | 4719 | CZ | ARG | A | 611 | 56.222 | 25.897 | 47.134 | 1.00 | 49.37 |
| ATOM | 4720 | NH1 | ARG | A | 611 | 57.180 | 25.503 | 47.966 | 1.00 | 51.26 |
| ATOM | 4721 | NH2 | ARG | A | 611 | 55.212 | 26.616 | 47.590 | 1.00 | 51.85 |
| ATOM | 4722 | N | GLN | A | 612 | 59.095 | 25.283 | 40.002 | 1.00 | 40.69 |
| ATOM | 4723 | CA | GLN | A | 612 | 59.295 | 24.516 | 38.788 | 1.00 | 42.53 |
| ATOM | 4724 | C | GLN | A | 612 | 60.617 | 24.888 | 38.130 | 1.00 | 43.64 |
| ATOM | 4725 | O | GLN | A | 612 | 61.286 | 24.045 | 37.494 | 1.00 | 44.69 |
| ATOM | 4726 | CB | GLN | A | 612 | 58.167 | 24.761 | 37.797 | 1.00 | 42.95 |
| ATOM | 4727 | CG | GLN | A | 612 | 56.828 | 24.301 | 38.290 | 1.00 | 44.37 |
| ATOM | 4728 | CD | GLN | A | 612 | 56.468 | 22.960 | 37.723 | 1.00 | 45.58 |
| ATOM | 4729 | OE1 | GLN | A | 612 | 56.577 | 22.774 | 36.523 | 1.00 | 49.79 |
| ATOM | 4730 | NE2 | GLN | A | 612 | 56.055 | 22.024 | 38.570 | 1.00 | 46.49 |
| ATOM | 4731 | N | PHE | A | 613 | 61.031 | 26.133 | 38.280 | 1.00 | 44.14 |
| ATOM | 4732 | CA | PHE | A | 613 | 62.286 | 26.495 | 37.660 | 1.00 | 44.66 |
| ATOM | 4733 | C | PHE | A | 613 | 63.380 | 25.812 | 38.424 | 1.00 | 46.53 |
| ATOM | 4734 | O | PHE | A | 613 | 64.423 | 25.517 | 37.859 | 1.00 | 46.58 |
| ATOM | 4735 | CB | PHE | A | 613 | 62.494 | 28.000 | 37.619 | 1.00 | 44.25 |
| ATOM | 4736 | CG | PHE | A | 613 | 61.499 | 28.723 | 36.770 | 1.00 | 42.36 |
| ATOM | 4737 | CD1 | PHE | A | 613 | 60.871 | 28.089 | 35.727 | 1.00 | 41.01 |
| ATOM | 4738 | CD2 | PHE | A | 613 | 61.187 | 30.039 | 37.024 | 1.00 | 41.38 |
| ATOM | 4739 | CE1 | PHE | A | 613 | 59.947 | 28.756 | 34.966 | 1.00 | 42.23 |
| ATOM | 4740 | CE2 | PHE | A | 613 | 60.273 | 30.704 | 36.251 | 1.00 | 41.19 |
| ATOM | 4741 | CZ | PHE | A | 613 | 59.652 | 30.063 | 35.227 | 1.00 | 40.85 |
| ATOM | 4742 | N | SER | A | 614 | 63.143 | 25.545 | 39.710 | 1.00 | 48.55 |
| ATOM | 4743 | CA | SER | A | 614 | 64.157 | 24.901 | 40.540 | 1.00 | 50.42 |
| ATOM | 4744 | C | SER | A | 614 | 64.372 | 23.447 | 40.118 | 1.00 | 52.01 |
| ATOM | 4745 | O | SER | A | 614 | 65.508 | 23.011 | 40.012 | 1.00 | 52.61 |
| ATOM | 4746 | CB | SER | A | 614 | 63.803 | 25.011 | 42.013 | 1.00 | 50.48 |
| ATOM | 4747 | OG | SER | A | 614 | 63.563 | 26.367 | 42.342 | 1.00 | 50.30 |
| ATOM | 4748 | N | LYS | A | 615 | 63.304 | 22.701 | 39.853 | 1.00 | 53.76 |
| ATOM | 4749 | CA | LYS | A | 615 | 63.475 | 21.333 | 39.354 | 1.00 | 54.92 |
| ATOM | 4750 | C | LYS | A | 615 | 64.531 | 21.398 | 38.257 | 1.00 | 55.07 |
| ATOM | 4751 | O | LYS | A | 615 | 65.654 | 20.886 | 38.439 | 1.00 | 55.88 |
| ATOM | 4752 | CB | LYS | A | 615 | 62.185 | 20.765 | 38.754 | 1.00 | 55.28 |
| ATOM | 4753 | CG | LYS | A | 615 | 61.109 | 20.315 | 39.739 | 1.00 | 57.19 |
| ATOM | 4754 | CD | LYS | A | 615 | 59.904 | 19.747 | 38.949 | 1.00 | 59.39 |
| ATOM | 4755 | CE | LYS | A | 615 | 58.582 | 19.712 | 39.737 | 1.00 | 60.46 |
| ATOM | 4756 | NZ | LYS | A | 615 | 57.504 | 18.979 | 38.966 | 1.00 | 60.49 |
| ATOM | 4757 | N | MET | A | 616 | 64.154 | 22.018 | 37.124 | 1.00 | 54.41 |
| ATOM | 4758 | CA | MET | A | 616 | 65.052 | 22.231 | 35.989 | 1.00 | 53.94 |
| ATOM | 4759 | C | MET | A | 616 | 66.436 | 22.449 | 36.593 | 1.00 | 52.94 |
| ATOM | 4760 | O | MET | A | 616 | 66.666 | 23.440 | 37.259 | 1.00 | 53.82 |
| ATOM | 4761 | CB | MET | A | 616 | 64.651 | 23.477 | 35.188 | 1.00 | 53.70 |
| ATOM | 4762 | CG | MET | A | 616 | 63.228 | 23.495 | 34.607 | 1.00 | 53.76 |
| ATOM | 4763 | SD | MET | A | 616 | 62.789 | 25.145 | 33.893 | 1.00 | 52.40 |
| ATOM | 4764 | CE | MET | A | 616 | 61.286 | 24.766 | 33.068 | 1.00 | 52.15 |
| ATOM | 4765 | N | GLY | A | 617 | 67.354 | 21.525 | 36.364 | 1.00 | 51.47 |
| ATOM | 4766 | CA | GLY | A | 617 | 68.633 | 21.534 | 37.053 | 1.00 | 49.40 |
| ATOM | 4767 | C | GLY | A | 617 | 69.663 | 22.605 | 36.777 | 1.00 | 48.16 |
| ATOM | 4768 | O | GLY | A | 617 | 70.841 | 22.369 | 37.016 | 1.00 | 48.41 |
| ATOM | 4769 | N | PHE | A | 618 | 69.287 | 23.791 | 36.306 | 1.00 | 46.53 |
| ATOM | 4770 | CA | PHE | A | 618 | 70.324 | 24.798 | 36.074 | 1.00 | 44.89 |
| ATOM | 4771 | C | PHE | A | 618 | 70.160 | 26.046 | 36.959 | 1.00 | 43.22 |
| ATOM | 4772 | O | PHE | A | 618 | 70.785 | 27.085 | 36.758 | 1.00 | 42.11 |
| ATOM | 4773 | CB | PHE | A | 618 | 70.412 | 25.149 | 34.595 | 1.00 | 44.90 |
| ATOM | 4774 | CG | PHE | A | 618 | 69.112 | 25.510 | 33.980 | 1.00 | 46.03 |
| ATOM | 4775 | CD1 | PHE | A | 618 | 68.587 | 26.780 | 34.142 | 1.00 | 47.22 |
| ATOM | 4776 | CD2 | PHE | A | 618 | 68.422 | 24.598 | 33.204 | 1.00 | 46.83 |
| ATOM | 4777 | CE1 | PHE | A | 618 | 67.381 | 27.120 | 33.564 | 1.00 | 46.69 |
| ATOM | 4778 | CE2 | PHE | A | 618 | 67.213 | 24.949 | 32.598 | 1.00 | 46.58 |
| ATOM | 4779 | CZ | PHE | A | 618 | 66.696 | 26.200 | 32.797 | 1.00 | 47.72 |
| ATOM | 4780 | N | VAL | A | 619 | 69.346 | 25.901 | 37.981 | 1.00 | 41.96 |
| ATOM | 4781 | CA | VAL | A | 619 | 69.077 | 26.980 | 38.871 | 1.00 | 41.17 |
| ATOM | 4782 | C | VAL | A | 619 | 69.578 | 26.625 | 40.240 | 1.00 | 40.50 |
| ATOM | 4783 | O | VAL | A | 619 | 69.354 | 25.522 | 40.721 | 1.00 | 39.68 |
| ATOM | 4784 | CB | VAL | A | 619 | 67.586 | 27.235 | 38.920 | 1.00 | 41.06 |
| ATOM | 4785 | CG1 | VAL | A | 619 | 67.224 | 28.165 | 40.078 | 1.00 | 40.61 |
| ATOM | 4786 | CG2 | VAL | A | 619 | 67.136 | 27.807 | 37.593 | 1.00 | 41.96 |
| ATOM | 4787 | N | ASP | A | 620 | 70.247 | 27.573 | 40.874 | 1.00 | 40.71 |
| ATOM | 4788 | CA | ASP | A | 620 | 70.709 | 27.386 | 42.251 | 1.00 | 41.12 |
| ATOM | 4789 | C | ASP | A | 620 | 69.556 | 27.739 | 43.152 | 1.00 | 40.94 |
| ATOM | 4790 | O | ASP | A | 620 | 69.176 | 28.901 | 43.251 | 1.00 | 40.18 |
| ATOM | 4791 | CB | ASP | A | 620 | 71.879 | 28.297 | 42.555 | 1.00 | 41.40 |
| ATOM | 4792 | CG | ASP | A | 620 | 72.267 | 28.286 | 44.023 | 1.00 | 42.62 |
| ATOM | 4793 | OD1 | ASP | A | 620 | 72.863 | 29.274 | 44.465 | 1.00 | 45.71 |
| ATOM | 4794 | OD2 | ASP | A | 620 | 72.035 | 27.352 | 44.813 | 1.00 | 46.38 |
| ATOM | 4795 | N | ASN | A | 621 | 68.988 | 26.777 | 43.849 | 1.00 | 41.25 |
| ATOM | 4796 | CA | ASN | A | 621 | 67.804 | 27.161 | 44.584 | 1.00 | 41.89 |
| ATOM | 4797 | C | ASN | A | 621 | 68.046 | 27.908 | 45.909 | 1.00 | 41.34 |
| ATOM | 4798 | O | ASN | A | 621 | 67.099 | 28.277 | 46.589 | 1.00 | 39.99 |
| ATOM | 4799 | CB | ASN | A | 621 | 66.766 | 26.036 | 44.645 | 1.00 | 43.11 |
| ATOM | 4800 | CG | ASN | A | 621 | 67.231 | 24.853 | 45.397 | 1.00 | 45.02 |
| ATOM | 4801 | OD1 | ASN | A | 621 | 67.014 | 23.714 | 44.973 | 1.00 | 50.87 |
| ATOM | 4802 | ND2 | ASN | A | 621 | 67.845 | 25.087 | 46.526 | 1.00 | 46.65 |
| ATOM | 4803 | N | LYS | A | 622 | 69.310 | 28.208 | 46.227 | 1.00 | 40.43 |
| ATOM | 4804 | CA | LYS | A | 622 | 69.589 | 29.069 | 47.385 | 1.00 | 39.96 |
| ATOM | 4805 | C | LYS | A | 622 | 69.584 | 30.538 | 46.948 | 1.00 | 38.41 |
| ATOM | 4806 | O | LYS | A | 622 | 69.594 | 31.442 | 47.777 | 1.00 | 38.04 |
| ATOM | 4807 | CB | LYS | A | 622 | 70.965 | 28.750 | 47.995 | 1.00 | 40.47 |
| ATOM | 4808 | CG | LYS | A | 622 | 71.167 | 27.286 | 48.347 | 1.00 | 43.46 |
| ATOM | 4809 | CD | LYS | A | 622 | 72.658 | 26.933 | 48.474 | 1.00 | 48.43 |
| ATOM | 4810 | CE | LYS | A | 622 | 72.827 | 25.462 | 48.888 | 1.00 | 50.37 |
| ATOM | 4811 | NZ | LYS | A | 622 | 74.269 | 25.016 | 48.993 | 1.00 | 54.19 |
| ATOM | 4812 | N | ARG | A | 623 | 69.596 | 30.773 | 45.639 | 1.00 | 36.15 |
| ATOM | 4813 | CA | ARG | A | 623 | 69.659 | 32.138 | 45.125 | 1.00 | 34.54 |
| ATOM | 4814 | C | ARG | A | 623 | 68.512 | 32.428 | 44.155 | 1.00 | 33.26 |
| ATOM | 4815 | O | ARG | A | 623 | 68.666 | 32.419 | 42.944 | 1.00 | 30.78 |
| ATOM | 4816 | CB | ARG | A | 623 | 71.044 | 32.401 | 44.507 | 1.00 | 34.09 |
| ATOM | 4817 | CG | ARG | A | 623 | 72.182 | 32.316 | 45.567 | 1.00 | 32.92 |
| ATOM | 4818 | CD | ARG | A | 623 | 73.528 | 32.690 | 45.050 | 1.00 | 33.79 |
| ATOM | 4819 | NE | ARG | A | 623 | 74.101 | 31.638 | 44.214 | 1.00 | 34.41 |
| ATOM | 4820 | CZ | ARG | A | 623 | 74.996 | 31.847 | 43.264 | 1.00 | 35.70 |
| ATOM | 4821 | NH1 | ARG | A | 623 | 75.434 | 33.083 | 43.002 | 1.00 | 33.42 |
| ATOM | 4822 | NH2 | ARG | A | 623 | 75.469 | 30.810 | 42.572 | 1.00 | 35.07 |
| ATOM | 4823 | N | ILE | A | 624 | 67.339 | 32.630 | 44.729 | 1.00 | 32.55 |
| ATOM | 4824 | CA | ILE | A | 624 | 66.173 | 33.001 | 43.941 | 1.00 | 32.44 |
| ATOM | 4825 | C | ILE | A | 624 | 65.601 | 34.288 | 44.517 | 1.00 | 31.62 |
| ATOM | 4826 | O | ILE | A | 624 | 65.414 | 34.462 | 45.733 | 1.00 | 30.99 |
| ATOM | 4827 | CB | ILE | A | 624 | 65.194 | 31.896 | 43.906 | 1.00 | 32.85 |
| ATOM | 4828 | CG1 | ILE | A | 624 | 65.898 | 30.628 | 43.386 | 1.00 | 34.14 |
| ATOM | 4829 | CG2 | ILE | A | 624 | 64.053 | 32.277 | 42.979 | 1.00 | 33.93 |
| ATOM | 4830 | CD1 | ILE | A | 624 | 64.961 | 29.405 | 43.207 | 1.00 | 36.13 |
| ATOM | 4831 | N | ALA | A | 625 | 65.415 | 35.240 | 43.638 | 1.00 | 30.01 |
| ATOM | 4832 | CA | ALA | A | 625 | 64.955 | 36.521 | 44.074 | 1.00 | 28.84 |
| ATOM | 4833 | C | ALA | A | 625 | 63.726 | 36.829 | 43.262 | 1.00 | 27.42 |
| ATOM | 4834 | O | ALA | A | 625 | 63.473 | 36.172 | 42.250 | 1.00 | 25.18 |
| ATOM | 4835 | CB | ALA | A | 625 | 66.014 | 37.558 | 43.807 | 1.00 | 28.63 |
| ATOM | 4836 | N | ILE | A | 626 | 63.021 | 37.872 | 43.677 | 1.00 | 26.42 |
| ATOM | 4837 | CA | ILE | A | 626 | 61.871 | 38.334 | 42.938 | 1.00 | 26.38 |
| ATOM | 4838 | C | ILE | A | 626 | 61.796 | 39.844 | 42.987 | 1.00 | 25.89 |
| ATOM | 4839 | O | ILE | A | 626 | 62.191 | 40.471 | 43.962 | 1.00 | 25.73 |
| ATOM | 4840 | CB | ILE | A | 626 | 60.623 | 37.689 | 43.494 | 1.00 | 26.45 |
| ATOM | 4841 | CG1 | ILE | A | 626 | 59.404 | 38.259 | 42.780 | 1.00 | 27.17 |
| ATOM | 4842 | CG2 | ILE | A | 626 | 60.566 | 37.887 | 45.004 | 1.00 | 26.71 |
| ATOM | 4843 | CD1 | ILE | A | 626 | 58.192 | 37.452 | 42.992 | 1.00 | 29.50 |
| ATOM | 4844 | N | TRP | A | 627 | 61.388 | 40.449 | 41.885 | 1.00 | 25.90 |
| ATOM | 4845 | CA | TRP | A | 627 | 61.195 | 41.883 | 41.870 | 1.00 | 25.90 |
| ATOM | 4846 | C | TRP | A | 627 | 60.116 | 42.345 | 40.938 | 1.00 | 26.04 |
| ATOM | 4847 | O | TRP | A | 627 | 59.762 | 41.680 | 39.959 | 1.00 | 25.99 |
| ATOM | 4848 | CB | TRP | A | 627 | 62.462 | 42.621 | 41.521 | 1.00 | 25.37 |
| ATOM | 4849 | CG | TRP | A | 627 | 62.614 | 42.982 | 40.096 | 1.00 | 26.40 |
| ATOM | 4850 | CD1 | TRP | A | 627 | 63.003 | 42.147 | 39.090 | 1.00 | 24.57 |
| ATOM | 4851 | CD2 | TRP | A | 627 | 62.464 | 44.288 | 39.505 | 1.00 | 24.31 |
| ATOM | 4852 | NE1 | TRP | A | 627 | 63.091 | 42.845 | 37.917 | 1.00 | 26.92 |
| ATOM | 4853 | CE2 | TRP | A | 627 | 62.768 | 44.160 | 38.139 | 1.00 | 24.75 |
| ATOM | 4854 | CE3 | TRP | A | 627 | 62.081 | 45.546 | 39.994 | 1.00 | 23.17 |
| ATOM | 4855 | CZ2 | TRP | A | 627 | 62.709 | 45.241 | 37.240 | 1.00 | 24.25 |
| ATOM | 4856 | CZ3 | TRP | A | 627 | 62.051 | 46.630 | 39.131 | 1.00 | 23.08 |
| ATOM | 4857 | CH2 | TRP | A | 627 | 62.350 | 46.473 | 37.744 | 1.00 | 23.04 |
| ATOM | 4858 | N | GLY | A | 628 | 59.619 | 43.538 | 41.219 | 1.00 | 25.40 |
| ATOM | 4859 | CA | GLY | A | 628 | 58.606 | 44.090 | 40.360 | 1.00 | 24.79 |
| ATOM | 4860 | C | GLY | A | 628 | 58.254 | 45.494 | 40.719 | 1.00 | 23.77 |
| ATOM | 4861 | O | GLY | A | 628 | 58.611 | 45.979 | 41.786 | 1.00 | 21.64 |
| ATOM | 4862 | N | TRP | A | 629 | 57.489 | 46.095 | 39.816 | 1.00 | 23.74 |
| ATOM | 4863 | CA | TRP | A | 629 | 57.087 | 47.503 | 39.854 | 1.00 | 24.09 |
| ATOM | 4864 | C | TRP | A | 629 | 55.580 | 47.499 | 39.761 | 1.00 | 23.81 |
| ATOM | 4865 | O | TRP | A | 629 | 55.006 | 46.713 | 39.018 | 1.00 | 23.65 |
| ATOM | 4866 | CB | TRP | A | 629 | 57.675 | 48.188 | 38.630 | 1.00 | 24.46 |
| ATOM | 4867 | CG | TRP | A | 629 | 57.929 | 49.640 | 38.721 | 1.00 | 25.53 |
| ATOM | 4868 | CD1 | TRP | A | 629 | 57.010 | 50.618 | 38.921 | 1.00 | 26.25 |
| ATOM | 4869 | CD2 | TRP | A | 629 | 59.186 | 50.315 | 38.506 | 1.00 | 23.91 |
| ATOM | 4870 | NE1 | TRP | A | 629 | 57.612 | 51.854 | 38.894 | 1.00 | 25.51 |
| ATOM | 4871 | CE2 | TRP | A | 629 | 58.952 | 51.692 | 38.636 | 1.00 | 26.50 |
| ATOM | 4872 | CE3 | TRP | A | 629 | 60.480 | 49.895 | 38.223 | 1.00 | 20.13 |
| ATOM | 4873 | CZ2 | TRP | A | 629 | 59.973 | 52.646 | 38.490 | 1.00 | 25.09 |
| ATOM | 4874 | CZ3 | TRP | A | 629 | 61.494 | 50.854 | 38.105 | 1.00 | 21.41 |
| ATOM | 4875 | CH2 | TRP | A | 629 | 61.233 | 52.194 | 38.231 | 1.00 | 21.98 |
| ATOM | 4876 | N | SER | A | 630 | 54.940 | 48.347 | 40.549 | 1.00 | 23.53 |
| ATOM | 4877 | CA | SER | A | 630 | 53.476 | 48.495 | 40.538 | 1.00 | 23.81 |
| ATOM | 4878 | C | SER | A | 630 | 52.706 | 47.239 | 40.961 | 1.00 | 23.46 |
| ATOM | 4879 | O | SER | A | 630 | 52.886 | 46.729 | 42.066 | 1.00 | 24.58 |
| ATOM | 4880 | CB | SER | A | 630 | 53.085 | 48.945 | 39.160 | 1.00 | 23.51 |
| ATOM | 4881 | OG | SER | A | 630 | 52.141 | 49.969 | 39.276 | 1.00 | 23.88 |
| ATOM | 4882 | N | TYR | A | 631 | 51.875 | 46.707 | 40.087 | 1.00 | 22.70 |
| ATOM | 4883 | CA | TYR | A | 631 | 51.241 | 45.474 | 40.384 | 1.00 | 21.81 |
| ATOM | 4884 | C | TYR | A | 631 | 52.357 | 44.489 | 40.697 | 1.00 | 22.55 |
| ATOM | 4885 | O | TYR | A | 631 | 52.188 | 43.602 | 41.532 | 1.00 | 21.39 |
| ATOM | 4886 | CB | TYR | A | 631 | 50.377 | 44.993 | 39.196 | 1.00 | 21.65 |
| ATOM | 4887 | CG | TYR | A | 631 | 49.347 | 44.009 | 39.632 | 1.00 | 21.44 |
| ATOM | 4888 | CD1 | TYR | A | 631 | 49.681 | 42.705 | 39.911 | 1.00 | 21.56 |
| ATOM | 4889 | CD2 | TYR | A | 631 | 48.049 | 44.406 | 39.883 | 1.00 | 24.94 |
| ATOM | 4890 | CE1 | TYR | A | 631 | 48.712 | 41.805 | 40.385 | 1.00 | 23.19 |
| ATOM | 4891 | CE2 | TYR | A | 631 | 47.076 | 43.499 | 40.331 | 1.00 | 24.52 |
| ATOM | 4892 | CZ | TYR | A | 631 | 47.414 | 42.205 | 40.586 | 1.00 | 23.00 |
| ATOM | 4893 | OH | TYR | A | 631 | 46.440 | 41.313 | 41.053 | 1.00 | 20.91 |
| ATOM | 4894 | N | GLY | A | 632 | 53.494 | 44.629 | 40.006 | 1.00 | 22.19 |
| ATOM | 4895 | CA | GLY | A | 632 | 54.627 | 43.746 | 40.239 | 1.00 | 23.91 |
| ATOM | 4896 | C | GLY | A | 632 | 55.255 | 43.912 | 41.627 | 1.00 | 23.98 |
| ATOM | 4897 | O | GLY | A | 632 | 55.741 | 42.950 | 42.239 | 1.00 | 23.20 |
| ATOM | 4898 | N | GLY | A | 633 | 55.236 | 45.145 | 42.130 | 1.00 | 24.94 |
| ATOM | 4899 | CA | GLY | A | 633 | 55.690 | 45.393 | 43.496 | 1.00 | 24.92 |
| ATOM | 4900 | C | GLY | A | 633 | 54.733 | 44.715 | 44.492 | 1.00 | 24.75 |
| ATOM | 4901 | O | GLY | A | 633 | 55.158 | 44.081 | 45.488 | 1.00 | 23.82 |
| ATOM | 4902 | N | TYR | A | 634 | 53.439 | 44.834 | 44.202 | 1.00 | 24.12 |
| ATOM | 4903 | CA | TYR | A | 634 | 52.419 | 44.239 | 45.024 | 1.00 | 23.97 |
| ATOM | 4904 | C | TYR | A | 634 | 52.605 | 42.715 | 45.065 | 1.00 | 24.59 |
| ATOM | 4905 | O | TYR | A | 634 | 52.688 | 42.100 | 46.134 | 1.00 | 24.79 |
| ATOM | 4906 | CB | TYR | A | 634 | 51.036 | 44.605 | 44.478 | 1.00 | 24.31 |
| ATOM | 4907 | CG | TYR | A | 634 | 49.889 | 43.855 | 45.111 | 1.00 | 23.82 |
| ATOM | 4908 | CD1 | TYR | A | 634 | 49.537 | 44.076 | 46.413 | 1.00 | 22.78 |
| ATOM | 4909 | CD2 | TYR | A | 634 | 49.141 | 42.944 | 44.381 | 1.00 | 21.52 |
| ATOM | 4910 | CE1 | TYR | A | 634 | 48.510 | 43.377 | 46.994 | 1.00 | 22.94 |
| ATOM | 4911 | CE2 | TYR | A | 634 | 48.089 | 42.282 | 44.941 | 1.00 | 22.09 |
| ATOM | 4912 | CZ | TYR | A | 634 | 47.766 | 42.496 | 46.246 | 1.00 | 22.45 |
| ATOM | 4913 | OH | TYR | A | 634 | 46.713 | 41.808 | 46.813 | 1.00 | 23.31 |
| ATOM | 4914 | N | VAL | A | 635 | 52.679 | 42.094 | 43.911 | 1.00 | 24.15 |
| ATOM | 4915 | CA | VAL | A | 635 | 52.865 | 40.652 | 43.888 | 1.00 | 24.87 |
| ATOM | 4916 | C | VAL | A | 635 | 54.173 | 40.241 | 44.517 | 1.00 | 24.65 |
| ATOM | 4917 | O | VAL | A | 635 | 54.235 | 39.251 | 45.199 | 1.00 | 24.18 |
| ATOM | 4918 | CB | VAL | A | 635 | 52.755 | 40.093 | 42.452 | 1.00 | 24.90 |
| ATOM | 4919 | CG1 | VAL | A | 635 | 53.081 | 38.613 | 42.426 | 1.00 | 25.11 |
| ATOM | 4920 | CG2 | VAL | A | 635 | 51.345 | 40.283 | 41.965 | 1.00 | 24.65 |
| ATOM | 4921 | N | THR | A | 636 | 55.221 | 41.004 | 44.284 | 1.00 | 25.12 |
| ATOM | 4922 | CA | THR | A | 636 | 56.512 | 40.709 | 44.864 | 1.00 | 25.36 |
| ATOM | 4923 | C | THR | A | 636 | 56.383 | 40.657 | 46.361 | 1.00 | 24.77 |
| ATOM | 4924 | O | THR | A | 636 | 56.873 | 39.736 | 47.028 | 1.00 | 24.50 |
| ATOM | 4925 | CB | THR | A | 636 | 57.531 | 41.829 | 44.459 | 1.00 | 26.95 |
| ATOM | 4926 | OG1 | THR | A | 636 | 58.035 | 41.606 | 43.126 | 1.00 | 26.06 |
| ATOM | 4927 | CG2 | THR | A | 636 | 58.823 | 41.791 | 45.315 | 1.00 | 26.91 |
| ATOM | 4928 | N | SER | A | 637 | 55.673 | 41.632 | 46.901 | 1.00 | 24.63 |
| ATOM | 4929 | CA | SER | A | 637 | 55.569 | 41.760 | 48.342 | 1.00 | 24.18 |
| ATOM | 4930 | C | SER | A | 637 | 54.662 | 40.683 | 48.920 | 1.00 | 25.28 |
| ATOM | 4931 | O | SER | A | 637 | 54.916 | 40.181 | 50.017 | 1.00 | 24.93 |
| ATOM | 4932 | CB | SER | A | 637 | 55.066 | 43.144 | 48.665 | 1.00 | 23.48 |
| ATOM | 4933 | OG | SER | A | 637 | 55.954 | 44.085 | 48.097 | 1.00 | 22.02 |
| ATOM | 4934 | N | MET | A | 638 | 53.631 | 40.292 | 48.167 | 1.00 | 24.69 |
| ATOM | 4935 | CA | MET | A | 638 | 52.741 | 39.262 | 48.643 | 1.00 | 24.72 |
| ATOM | 4936 | C | MET | A | 638 | 53.465 | 37.929 | 48.646 | 1.00 | 24.85 |
| ATOM | 4937 | O | MET | A | 638 | 53.264 | 37.130 | 49.549 | 1.00 | 23.44 |
| ATOM | 4938 | CB | MET | A | 638 | 51.476 | 39.197 | 47.775 | 1.00 | 25.04 |
| ATOM | 4939 | CG | MET | A | 638 | 50.589 | 40.396 | 47.989 | 1.00 | 25.97 |
| ATOM | 4940 | SD | MET | A | 638 | 49.706 | 40.451 | 49.503 | 1.00 | 24.26 |
| ATOM | 4941 | CE | MET | A | 638 | 48.273 | 39.399 | 49.121 | 1.00 | 28.34 |
| ATOM | 4942 | N | VAL | A | 639 | 54.286 | 37.675 | 47.628 | 1.00 | 24.38 |
| ATOM | 4943 | CA | VAL | A | 639 | 55.119 | 36.483 | 47.614 | 1.00 | 25.85 |
| ATOM | 4944 | C | VAL | A | 639 | 56.196 | 36.492 | 48.717 | 1.00 | 26.46 |
| ATOM | 4945 | O | VAL | A | 639 | 56.373 | 35.522 | 49.392 | 1.00 | 27.64 |
| ATOM | 4946 | CB | VAL | A | 639 | 55.908 | 36.344 | 46.272 | 1.00 | 26.72 |
| ATOM | 4947 | CG1 | VAL | A | 639 | 56.962 | 35.246 | 46.381 | 1.00 | 26.16 |
| ATOM | 4948 | CG2 | VAL | A | 639 | 54.979 | 36.090 | 45.121 | 1.00 | 27.54 |
| ATOM | 4949 | N | LEU | A | 640 | 56.939 | 37.573 | 48.899 | 1.00 | 27.30 |
| ATOM | 4950 | CA | LEU | A | 640 | 57.951 | 37.538 | 49.936 | 1.00 | 28.97 |
| ATOM | 4951 | C | LEU | A | 640 | 57.278 | 37.246 | 51.260 | 1.00 | 30.01 |
| ATOM | 4952 | O | LEU | A | 640 | 57.859 | 36.559 | 52.084 | 1.00 | 30.46 |
| ATOM | 4953 | CB | LEU | A | 640 | 58.738 | 38.831 | 50.055 | 1.00 | 28.39 |
| ATOM | 4954 | CG | LEU | A | 640 | 59.541 | 39.123 | 48.818 | 1.00 | 30.68 |
| ATOM | 4955 | CD1 | LEU | A | 640 | 59.983 | 40.560 | 48.808 | 1.00 | 31.22 |
| ATOM | 4956 | CD2 | LEU | A | 640 | 60.717 | 38.152 | 48.770 | 1.00 | 32.89 |
| ATOM | 4957 | N | GLY | A | 641 | 56.060 | 37.755 | 51.445 | 1.00 | 30.64 |
| ATOM | 4958 | CA | GLY | A | 641 | 55.335 | 37.561 | 52.683 | 1.00 | 31.58 |
| ATOM | 4959 | C | GLY | A | 641 | 54.415 | 36.363 | 52.781 | 1.00 | 32.11 |
| ATOM | 4960 | O | GLY | A | 641 | 53.599 | 36.272 | 53.722 | 1.00 | 32.54 |
| ATOM | 4961 | N | SER | A | 642 | 54.541 | 35.419 | 51.854 | 1.00 | 31.43 |
| ATOM | 4962 | CA | SER | A | 642 | 53.673 | 34.249 | 51.887 | 1.00 | 30.86 |
| ATOM | 4963 | C | SER | A | 642 | 54.255 | 33.136 | 52.764 | 1.00 | 31.44 |
| ATOM | 4964 | O | SER | A | 642 | 53.576 | 32.123 | 53.033 | 1.00 | 31.55 |
| ATOM | 4965 | CB | SER | A | 642 | 53.543 | 33.701 | 50.471 | 1.00 | 30.30 |
| ATOM | 4966 | OG | SER | A | 642 | 54.803 | 33.191 | 50.091 | 1.00 | 28.39 |
| ATOM | 4967 | N | GLY | A | 643 | 55.517 | 33.299 | 53.165 | 1.00 | 31.79 |
| ATOM | 4968 | CA | GLY | A | 643 | 56.219 | 32.290 | 53.944 | 1.00 | 32.28 |
| ATOM | 4969 | C | GLY | A | 643 | 56.597 | 31.034 | 53.160 | 1.00 | 32.91 |
| ATOM | 4970 | O | GLY | A | 643 | 56.811 | 29.976 | 53.738 | 1.00 | 32.68 |
| ATOM | 4971 | N | SER | A | 644 | 56.717 | 31.140 | 51.843 | 1.00 | 33.20 |
| ATOM | 4972 | CA | SER | A | 644 | 57.001 | 29.960 | 51.022 | 1.00 | 33.35 |
| ATOM | 4973 | C | SER | A | 644 | 58.383 | 29.357 | 51.263 | 1.00 | 33.95 |
| ATOM | 4974 | O | SER | A | 644 | 58.575 | 28.159 | 51.086 | 1.00 | 33.80 |
| ATOM | 4975 | CB | SER | A | 644 | 56.890 | 30.322 | 49.557 | 1.00 | 33.20 |
| ATOM | 4976 | OG | SER | A | 644 | 58.097 | 30.922 | 49.129 | 1.00 | 33.53 |
| ATOM | 4977 | N | GLY | A | 645 | 59.340 | 30.193 | 51.651 | 1.00 | 33.66 |
| ATOM | 4978 | CA | GLY | A | 645 | 60.710 | 29.748 | 51.844 | 1.00 | 33.54 |
| ATOM | 4979 | C | GLY | A | 645 | 61.443 | 29.611 | 50.508 | 1.00 | 33.70 |
| ATOM | 4980 | O | GLY | A | 645 | 62.651 | 29.390 | 50.463 | 1.00 | 32.41 |
| ATOM | 4981 | N | VAL | A | 646 | 60.714 | 29.802 | 49.411 | 1.00 | 33.25 |
| ATOM | 4982 | CA | VAL | A | 646 | 61.314 | 29.684 | 48.086 | 1.00 | 33.17 |
| ATOM | 4983 | C | VAL | A | 646 | 62.227 | 30.858 | 47.752 | 1.00 | 32.53 |
| ATOM | 4984 | O | VAL | A | 646 | 63.240 | 30.663 | 47.099 | 1.00 | 33.19 |
| ATOM | 4985 | CB | VAL | A | 646 | 60.226 | 29.583 | 46.990 | 1.00 | 33.15 |
| ATOM | 4986 | CG1 | VAL | A | 646 | 60.849 | 29.618 | 45.576 | 1.00 | 33.99 |
| ATOM | 4987 | CG2 | VAL | A | 646 | 59.397 | 28.324 | 47.195 | 1.00 | 33.67 |
| ATOM | 4988 | N | PHE | A | 647 | 61.884 | 32.075 | 48.168 | 1.00 | 31.56 |
| ATOM | 4989 | CA | PHE | A | 647 | 62.712 | 33.216 | 47.773 | 1.00 | 30.96 |
| ATOM | 4990 | C | PHE | A | 647 | 63.624 | 33.734 | 48.881 | 1.00 | 30.94 |
| ATOM | 4991 | O | PHE | A | 647 | 63.248 | 33.746 | 50.065 | 1.00 | 30.19 |
| ATOM | 4992 | CB | PHE | A | 647 | 61.856 | 34.356 | 47.230 | 1.00 | 30.77 |
| ATOM | 4993 | CG | PHE | A | 647 | 60.940 | 33.951 | 46.099 | 1.00 | 30.66 |
| ATOM | 4994 | CD1 | PHE | A | 647 | 59.737 | 33.315 | 46.354 | 1.00 | 29.51 |
| ATOM | 4995 | CD2 | PHE | A | 647 | 61.290 | 34.223 | 44.789 | 1.00 | 27.92 |
| ATOM | 4996 | CE1 | PHE | A | 647 | 58.887 | 32.943 | 45.286 | 1.00 | 32.03 |
| ATOM | 4997 | CE2 | PHE | A | 647 | 60.466 | 33.866 | 43.755 | 1.00 | 30.57 |
| ATOM | 4998 | CZ | PHE | A | 647 | 59.261 | 33.223 | 43.991 | 1.00 | 27.73 |
| ATOM | 4999 | N | LYS | A | 648 | 64.815 | 34.169 | 48.474 | 1.00 | 30.78 |
| ATOM | 5000 | CA | LYS | A | 648 | 65.806 | 34.693 | 49.399 | 1.00 | 31.48 |
| ATOM | 5001 | C | LYS | A | 648 | 65.645 | 36.169 | 49.604 | 1.00 | 31.25 |
| ATOM | 5002 | O | LYS | A | 648 | 65.859 | 36.687 | 50.675 | 1.00 | 30.03 |
| ATOM | 5003 | CB | LYS | A | 648 | 67.221 | 34.458 | 48.881 | 1.00 | 32.00 |
| ATOM | 5004 | CG | LYS | A | 648 | 68.309 | 34.871 | 49.892 | 1.00 | 30.90 |
| ATOM | 5005 | CD | LYS | A | 648 | 69.674 | 34.331 | 49.513 | 1.00 | 31.40 |
| ATOM | 5006 | CE | LYS | A | 648 | 70.674 | 34.600 | 50.627 | 1.00 | 31.69 |
| ATOM | 5007 | NZ | LYS | A | 648 | 71.597 | 35.694 | 50.288 | 1.00 | 34.00 |
| ATOM | 5008 | N | CYS | A | 649 | 65.267 | 36.857 | 48.546 | 1.00 | 32.32 |
| ATOM | 5009 | CA | CYS | A | 649 | 65.195 | 38.300 | 48.597 | 1.00 | 32.89 |
| ATOM | 5010 | C | CYS | A | 649 | 64.291 | 38.773 | 47.485 | 1.00 | 32.18 |
| ATOM | 5011 | O | CYS | A | 649 | 63.949 | 37.991 | 46.601 | 1.00 | 32.39 |
| ATOM | 5012 | CB | CYS | A | 649 | 66.594 | 38.878 | 48.433 | 1.00 | 33.39 |
| ATOM | 5013 | SG | CYS | A | 649 | 67.424 | 38.414 | 46.901 | 1.00 | 38.32 |
| ATOM | 5014 | N | GLY | A | 650 | 63.907 | 40.043 | 47.536 | 1.00 | 30.82 |
| ATOM | 5015 | CA | GLY | A | 650 | 63.102 | 40.625 | 46.489 | 1.00 | 29.96 |
| ATOM | 5016 | C | GLY | A | 650 | 62.993 | 42.133 | 46.627 | 1.00 | 29.16 |
| ATOM | 5017 | O | GLY | A | 650 | 63.251 | 42.698 | 47.702 | 1.00 | 27.45 |
| ATOM | 5018 | N | ILE | A | 651 | 62.593 | 42.781 | 45.534 | 1.00 | 27.34 |
| ATOM | 5019 | CA | ILE | A | 651 | 62.516 | 44.223 | 45.489 | 1.00 | 26.82 |
| ATOM | 5020 | C | ILE | A | 651 | 61.156 | 44.660 | 44.990 | 1.00 | 26.67 |
| ATOM | 5021 | O | ILE | A | 651 | 60.721 | 44.216 | 43.920 | 1.00 | 26.22 |
| ATOM | 5022 | CB | ILE | A | 651 | 63.526 | 44.765 | 44.531 | 1.00 | 27.03 |
| ATOM | 5023 | CG1 | ILE | A | 651 | 64.910 | 44.190 | 44.820 | 1.00 | 27.69 |
| ATOM | 5024 | CG2 | ILE | A | 651 | 63.528 | 46.266 | 44.570 | 1.00 | 27.62 |
| ATOM | 5025 | CD1 | ILE | A | 651 | 65.992 | 44.754 | 43.919 | 1.00 | 27.67 |
| ATOM | 5026 | N | ALA | A | 652 | 60.529 | 45.576 | 45.726 | 1.00 | 25.42 |
| ATOM | 5027 | CA | ALA | A | 652 | 59.212 | 46.085 | 45.369 | 1.00 | 25.10 |
| ATOM | 5028 | C | ALA | A | 652 | 59.287 | 47.556 | 45.063 | 1.00 | 24.43 |
| ATOM | 5029 | O | ALA | A | 652 | 59.646 | 48.354 | 45.922 | 1.00 | 22.80 |
| ATOM | 5030 | CB | ALA | A | 652 | 58.224 | 45.831 | 46.519 | 1.00 | 24.99 |
| ATOM | 5031 | N | VAL | A | 653 | 58.928 | 47.924 | 43.831 | 1.00 | 23.92 |
| ATOM | 5032 | CA | VAL | A | 653 | 58.965 | 49.319 | 43.441 | 1.00 | 23.46 |
| ATOM | 5033 | C | VAL | A | 653 | 57.594 | 49.880 | 43.243 | 1.00 | 23.10 |
| ATOM | 5034 | O | VAL | A | 653 | 56.831 | 49.360 | 42.421 | 1.00 | 24.71 |
| ATOM | 5035 | CB | VAL | A | 653 | 59.806 | 49.499 | 42.187 | 1.00 | 23.90 |
| ATOM | 5036 | CG1 | VAL | A | 653 | 59.927 | 50.968 | 41.798 | 1.00 | 21.62 |
| ATOM | 5037 | CG2 | VAL | A | 653 | 61.153 | 48.864 | 42.415 | 1.00 | 24.79 |
| ATOM | 5038 | N | ALA | A | 654 | 57.313 | 50.956 | 43.970 | 1.00 | 21.46 |
| ATOM | 5039 | CA | ALA | A | 654 | 56.044 | 51.634 | 43.964 | 1.00 | 21.63 |
| ATOM | 5040 | C | ALA | A | 654 | 54.883 | 50.657 | 43.980 | 1.00 | 22.36 |
| ATOM | 5041 | O | ALA | A | 654 | 54.016 | 50.702 | 43.148 | 1.00 | 22.12 |
| ATOM | 5042 | CB | ALA | A | 654 | 55.930 | 52.551 | 42.754 | 1.00 | 21.62 |
| ATOM | 5043 | N | PRO | A | 655 | 54.846 | 49.788 | 44.962 | 1.00 | 23.37 |
| ATOM | 5044 | CA | PRO | A | 655 | 53.793 | 48.772 | 45.019 | 1.00 | 23.85 |
| ATOM | 5045 | C | PRO | A | 655 | 52.444 | 49.278 | 45.479 | 1.00 | 24.40 |
| ATOM | 5046 | O | PRO | A | 655 | 52.332 | 50.249 | 46.245 | 1.00 | 23.99 |
| ATOM | 5047 | CB | PRO | A | 655 | 54.311 | 47.794 | 46.052 | 1.00 | 24.57 |
| ATOM | 5048 | CG | PRO | A | 655 | 55.242 | 48.635 | 46.918 | 1.00 | 24.95 |
| ATOM | 5049 | CD | PRO | A | 655 | 55.787 | 49.731 | 46.087 | 1.00 | 22.29 |
| ATOM | 5050 | N | VAL | A | 656 | 51.397 | 48.633 | 44.966 | 1.00 | 24.08 |
| ATOM | 5051 | CA | VAL | A | 656 | 50.092 | 48.801 | 45.555 | 1.00 | 23.00 |
| ATOM | 5052 | C | VAL | A | 656 | 50.242 | 47.927 | 46.766 | 1.00 | 23.22 |
| ATOM | 5053 | O | VAL | A | 656 | 50.901 | 46.907 | 46.654 | 1.00 | 23.66 |
| ATOM | 5054 | CB | VAL | A | 656 | 48.996 | 48.248 | 44.633 | 1.00 | 23.42 |
| ATOM | 5055 | CG1 | VAL | A | 656 | 47.831 | 47.830 | 45.408 | 1.00 | 22.09 |
| ATOM | 5056 | CG2 | VAL | A | 656 | 48.581 | 49.280 | 43.637 | 1.00 | 21.87 |
| ATOM | 5057 | N | SER | A | 657 | 49.708 | 48.314 | 47.928 | 1.00 | 22.68 |
| ATOM | 5058 | CA | SER | A | 657 | 49.749 | 47.460 | 49.139 | 1.00 | 22.65 |
| ATOM | 5059 | C | SER | A | 657 | 48.381 | 47.086 | 49.698 | 1.00 | 22.62 |
| ATOM | 5060 | O | SER | A | 657 | 48.314 | 46.142 | 50.476 | 1.00 | 24.25 |
| ATOM | 5061 | CB | SER | A | 657 | 50.497 | 48.154 | 50.306 | 1.00 | 22.94 |
| ATOM | 5062 | OG | SER | A | 657 | 49.785 | 49.330 | 50.750 | 1.00 | 21.12 |
| ATOM | 5063 | N | ARG | A | 658 | 47.322 | 47.821 | 49.328 | 1.00 | 22.76 |
| ATOM | 5064 | CA | ARG | A | 658 | 45.960 | 47.573 | 49.773 | 1.00 | 23.64 |
| ATOM | 5065 | C | ARG | A | 658 | 45.028 | 48.259 | 48.770 | 1.00 | 23.86 |
| ATOM | 5066 | O | ARG | A | 658 | 45.194 | 49.447 | 48.447 | 1.00 | 23.24 |
| ATOM | 5067 | CB | ARG | A | 658 | 45.789 | 48.118 | 51.197 | 1.00 | 24.97 |
| ATOM | 5068 | CG | ARG | A | 658 | 44.450 | 48.173 | 51.828 | 1.00 | 27.68 |
| ATOM | 5069 | CD | ARG | A | 658 | 44.608 | 48.584 | 53.292 | 1.00 | 32.89 |
| ATOM | 5070 | NE | ARG | A | 658 | 43.487 | 48.394 | 54.210 | 1.00 | 39.23 |
| ATOM | 5071 | CZ | ARG | A | 658 | 42.515 | 49.260 | 54.412 | 1.00 | 40.71 |
| ATOM | 5072 | NH1 | ARG | A | 658 | 42.437 | 50.367 | 53.692 | 1.00 | 42.58 |
| ATOM | 5073 | NH2 | ARG | A | 658 | 41.585 | 48.998 | 55.307 | 1.00 | 40.26 |
| ATOM | 5074 | N | TRP | A | 659 | 44.032 | 47.502 | 48.300 | 1.00 | 22.99 |
| ATOM | 5075 | CA | TRP | A | 659 | 43.247 | 47.925 | 47.165 | 1.00 | 22.51 |
| ATOM | 5076 | C | TRP | A | 659 | 42.364 | 49.102 | 47.479 | 1.00 | 22.51 |
| ATOM | 5077 | O | TRP | A | 659 | 42.112 | 49.894 | 46.602 | 1.00 | 21.24 |
| ATOM | 5078 | CB | TRP | A | 659 | 42.505 | 46.741 | 46.563 | 1.00 | 22.25 |
| ATOM | 5079 | CG | TRP | A | 659 | 43.443 | 45.839 | 45.961 | 1.00 | 21.75 |
| ATOM | 5080 | CD1 | TRP | A | 659 | 43.805 | 44.591 | 46.380 | 1.00 | 21.07 |
| ATOM | 5081 | CD2 | TRP | A | 659 | 44.200 | 46.103 | 44.790 | 1.00 | 19.12 |
| ATOM | 5082 | NE1 | TRP | A | 659 | 44.761 | 44.068 | 45.530 | 1.00 | 20.37 |
| ATOM | 5083 | CE2 | TRP | A | 659 | 45.031 | 44.991 | 44.560 | 1.00 | 20.52 |
| ATOM | 5084 | CE3 | TRP | A | 659 | 44.288 | 47.190 | 43.930 | 1.00 | 20.26 |
| ATOM | 5085 | CZ2 | TRP | A | 659 | 45.900 | 44.922 | 43.474 | 1.00 | 21.88 |
| ATOM | 5086 | CZ3 | TRP | A | 659 | 45.162 | 47.129 | 42.866 | 1.00 | 21.87 |
| ATOM | 5087 | CH2 | TRP | A | 659 | 45.936 | 45.993 | 42.640 | 1.00 | 21.25 |
| ATOM | 5088 | N | GLU | A | 660 | 41.981 | 49.279 | 48.738 | 1.00 | 23.00 |
| ATOM | 5089 | CA | GLU | A | 660 | 41.224 | 50.461 | 49.126 | 1.00 | 22.39 |
| ATOM | 5090 | C | GLU | A | 660 | 42.084 | 51.714 | 48.900 | 1.00 | 23.18 |
| ATOM | 5091 | O | GLU | A | 660 | 41.554 | 52.827 | 48.778 | 1.00 | 23.78 |
| ATOM | 5092 | CB | GLU | A | 660 | 40.676 | 50.387 | 50.588 | 1.00 | 24.05 |
| ATOM | 5093 | CG | GLU | A | 660 | 39.392 | 49.556 | 50.770 | 1.00 | 26.09 |
| ATOM | 5094 | CD | GLU | A | 660 | 39.262 | 48.942 | 52.189 | 1.00 | 30.94 |
| ATOM | 5095 | OE1 | GLU | A | 660 | 39.852 | 47.858 | 52.484 | 1.00 | 29.34 |
| ATOM | 5096 | OE2 | GLU | A | 660 | 38.555 | 49.524 | 53.046 | 1.00 | 35.02 |
| ATOM | 5097 | N | TYR | A | 661 | 43.395 | 51.579 | 48.777 | 1.00 | 22.69 |
| ATOM | 5098 | CA | TYR | A | 661 | 44.173 | 52.761 | 48.476 | 1.00 | 22.95 |
| ATOM | 5099 | C | TYR | A | 661 | 44.228 | 53.126 | 46.967 | 1.00 | 22.89 |
| ATOM | 5100 | O | TYR | A | 661 | 44.621 | 54.239 | 46.651 | 1.00 | 21.39 |
| ATOM | 5101 | CB | TYR | A | 661 | 45.611 | 52.589 | 48.930 | 1.00 | 22.98 |
| ATOM | 5102 | CG | TYR | A | 661 | 45.819 | 52.433 | 50.422 | 1.00 | 23.13 |
| ATOM | 5103 | CD1 | TYR | A | 661 | 44.956 | 53.014 | 51.351 | 1.00 | 24.47 |
| ATOM | 5104 | CD2 | TYR | A | 661 | 46.887 | 51.717 | 50.905 | 1.00 | 21.26 |
| ATOM | 5105 | CE1 | TYR | A | 661 | 45.166 | 52.840 | 52.693 | 1.00 | 24.70 |
| ATOM | 5106 | CE2 | TYR | A | 661 | 47.100 | 51.566 | 52.259 | 1.00 | 20.68 |
| ATOM | 5107 | CZ | TYR | A | 661 | 46.248 | 52.117 | 53.144 | 1.00 | 22.08 |
| ATOM | 5108 | OH | TYR | A | 661 | 46.446 | 51.935 | 54.523 | 1.00 | 21.51 |
| ATOM | 5109 | N | TYR | A | 662 | 43.906 | 52.192 | 46.063 | 1.00 | 21.57 |
| ATOM | 5110 | CA | TYR | A | 662 | 44.091 | 52.436 | 44.647 | 1.00 | 21.52 |
| ATOM | 5111 | C | TYR | A | 662 | 42.828 | 53.020 | 43.979 | 1.00 | 22.38 |
| ATOM | 5112 | O | TYR | A | 662 | 41.761 | 53.079 | 44.599 | 1.00 | 22.23 |
| ATOM | 5113 | CB | TYR | A | 662 | 44.657 | 51.187 | 43.933 | 1.00 | 22.04 |
| ATOM | 5114 | CG | TYR | A | 662 | 45.273 | 51.547 | 42.627 | 1.00 | 21.82 |
| ATOM | 5115 | CD1 | TYR | A | 662 | 46.229 | 52.581 | 42.568 | 1.00 | 22.72 |
| ATOM | 5116 | CD2 | TYR | A | 662 | 44.853 | 50.960 | 41.439 | 1.00 | 21.25 |
| ATOM | 5117 | CE1 | TYR | A | 662 | 46.760 | 52.983 | 41.386 | 1.00 | 21.34 |
| ATOM | 5118 | CE2 | TYR | A | 662 | 45.427 | 51.326 | 40.236 | 1.00 | 25.30 |
| ATOM | 5119 | CZ | TYR | A | 662 | 46.370 | 52.345 | 40.214 | 1.00 | 23.50 |
| ATOM | 5120 | OH | TYR | A | 662 | 46.898 | 52.771 | 39.048 | 1.00 | 20.23 |
| ATOM | 5121 | N | ASP | A | 663 | 42.922 | 53.492 | 42.736 | 1.00 | 22.24 |
| ATOM | 5122 | CA | ASP | A | 663 | 41.808 | 54.237 | 42.199 | 1.00 | 22.59 |
| ATOM | 5123 | C | ASP | A | 663 | 40.605 | 53.355 | 41.901 | 1.00 | 23.66 |
| ATOM | 5124 | O | ASP | A | 663 | 40.719 | 52.120 | 41.715 | 1.00 | 23.06 |
| ATOM | 5125 | CB | ASP | A | 663 | 42.203 | 55.065 | 40.984 | 1.00 | 23.04 |
| ATOM | 5126 | CG | ASP | A | 663 | 42.439 | 54.223 | 39.732 | 1.00 | 24.88 |
| ATOM | 5127 | OD1 | ASP | A | 663 | 41.517 | 53.513 | 39.230 | 1.00 | 26.15 |
| ATOM | 5128 | OD2 | ASP | A | 663 | 43.539 | 54.229 | 39.179 | 1.00 | 25.50 |
| ATOM | 5129 | N | SER | A | 664 | 39.454 | 54.015 | 41.894 | 1.00 | 23.69 |
| ATOM | 5130 | CA | SER | A | 664 | 38.199 | 53.343 | 41.685 | 1.00 | 24.34 |
| ATOM | 5131 | C | SER | A | 664 | 38.080 | 52.588 | 40.375 | 1.00 | 23.92 |
| ATOM | 5132 | O | SER | A | 664 | 37.619 | 51.473 | 40.397 | 1.00 | 23.78 |
| ATOM | 5133 | CB | SER | A | 664 | 37.065 | 54.334 | 41.743 | 1.00 | 24.41 |
| ATOM | 5134 | OG | SER | A | 664 | 37.255 | 55.329 | 40.782 | 1.00 | 24.35 |
| ATOM | 5135 | N | VAL | A | 665 | 38.446 | 53.195 | 39.253 | 1.00 | 23.95 |
| ATOM | 5136 | CA | VAL | A | 665 | 38.176 | 52.557 | 37.959 | 1.00 | 24.43 |
| ATOM | 5137 | C | VAL | A | 665 | 38.901 | 51.223 | 37.779 | 1.00 | 24.19 |
| ATOM | 5138 | O | VAL | A | 665 | 38.343 | 50.255 | 37.338 | 1.00 | 25.04 |
| ATOM | 5139 | CB | VAL | A | 665 | 38.510 | 53.485 | 36.775 | 1.00 | 25.20 |
| ATOM | 5140 | CG1 | VAL | A | 665 | 38.307 | 52.741 | 35.374 | 1.00 | 23.28 |
| ATOM | 5141 | CG2 | VAL | A | 665 | 37.629 | 54.748 | 36.854 | 1.00 | 26.35 |
| ATOM | 5142 | N | TYR | A | 666 | 40.155 | 51.172 | 38.130 | 1.00 | 23.72 |
| ATOM | 5143 | CA | TYR | A | 666 | 40.910 | 49.952 | 37.933 | 1.00 | 24.29 |
| ATOM | 5144 | C | TYR | A | 666 | 40.585 | 48.956 | 38.982 | 1.00 | 23.81 |
| ATOM | 5145 | O | TYR | A | 666 | 40.384 | 47.791 | 38.677 | 1.00 | 22.28 |
| ATOM | 5146 | CB | TYR | A | 666 | 42.382 | 50.267 | 38.030 | 1.00 | 24.47 |
| ATOM | 5147 | CG | TYR | A | 666 | 43.301 | 49.105 | 37.900 | 1.00 | 25.39 |
| ATOM | 5148 | CD1 | TYR | A | 666 | 43.624 | 48.336 | 38.997 | 1.00 | 27.84 |
| ATOM | 5149 | CD2 | TYR | A | 666 | 43.861 | 48.776 | 36.670 | 1.00 | 25.64 |
| ATOM | 5150 | CE1 | TYR | A | 666 | 44.493 | 47.296 | 38.891 | 1.00 | 26.70 |
| ATOM | 5151 | CE2 | TYR | A | 666 | 44.715 | 47.730 | 36.545 | 1.00 | 25.37 |
| ATOM | 5152 | CZ | TYR | A | 666 | 45.055 | 47.009 | 37.667 | 1.00 | 25.40 |
| ATOM | 5153 | OH | TYR | A | 666 | 45.894 | 45.942 | 37.574 | 1.00 | 24.41 |
| ATOM | 5154 | N | THR | A | 667 | 40.531 | 49.419 | 40.234 | 1.00 | 24.01 |
| ATOM | 5155 | CA | THR | A | 667 | 40.355 | 48.504 | 41.354 | 1.00 | 24.46 |
| ATOM | 5156 | C | THR | A | 667 | 38.998 | 47.836 | 41.314 | 1.00 | 25.21 |
| ATOM | 5157 | O | THR | A | 667 | 38.896 | 46.605 | 41.452 | 1.00 | 26.15 |
| ATOM | 5158 | CB | THR | A | 667 | 40.529 | 49.220 | 42.736 | 1.00 | 24.60 |
| ATOM | 5159 | OG1 | THR | A | 667 | 41.790 | 49.890 | 42.820 | 1.00 | 24.03 |
| ATOM | 5160 | CG2 | THR | A | 667 | 40.590 | 48.214 | 43.886 | 1.00 | 24.98 |
| ATOM | 5161 | N | GLU | A | 668 | 37.949 | 48.627 | 41.147 | 1.00 | 25.25 |
| ATOM | 5162 | CA | GLU | A | 668 | 36.603 | 48.073 | 41.246 | 1.00 | 25.97 |
| ATOM | 5163 | C | GLU | A | 668 | 36.276 | 47.103 | 40.084 | 1.00 | 26.85 |
| ATOM | 5164 | O | GLU | A | 668 | 35.475 | 46.201 | 40.206 | 1.00 | 26.02 |
| ATOM | 5165 | CB | GLU | A | 668 | 35.569 | 49.207 | 41.358 | 1.00 | 26.00 |
| ATOM | 5166 | CG | GLU | A | 668 | 35.673 | 49.996 | 42.671 | 1.00 | 25.76 |
| ATOM | 5167 | CD | GLU | A | 668 | 34.948 | 51.309 | 42.602 | 1.00 | 26.41 |
| ATOM | 5168 | OE1 | GLU | A | 668 | 34.045 | 51.443 | 41.722 | 1.00 | 28.15 |
| ATOM | 5169 | OE2 | GLU | A | 668 | 35.267 | 52.208 | 43.394 | 1.00 | 25.22 |
| ATOM | 5170 | N | ARG | A | 669 | 36.964 | 47.271 | 38.974 | 1.00 | 28.90 |
| ATOM | 5171 | CA | ARG | A | 669 | 36.802 | 46.366 | 37.844 | 1.00 | 30.04 |
| ATOM | 5172 | C | ARG | A | 669 | 37.070 | 44.946 | 38.254 | 1.00 | 29.89 |
| ATOM | 5173 | O | ARG | A | 669 | 36.313 | 44.034 | 37.940 | 1.00 | 28.41 |
| ATOM | 5174 | CB | ARG | A | 669 | 37.853 | 46.695 | 36.804 | 1.00 | 30.81 |
| ATOM | 5175 | CG | ARG | A | 669 | 37.285 | 46.953 | 35.499 | 1.00 | 34.76 |
| ATOM | 5176 | CD | ARG | A | 669 | 37.998 | 46.346 | 34.354 | 1.00 | 36.11 |
| ATOM | 5177 | NE | ARG | A | 669 | 39.380 | 46.723 | 34.302 | 1.00 | 38.37 |
| ATOM | 5178 | CZ | ARG | A | 669 | 39.849 | 47.943 | 34.131 | 1.00 | 38.35 |
| ATOM | 5179 | NH1 | ARG | A | 669 | 39.055 | 48.979 | 33.944 | 1.00 | 38.09 |
| ATOM | 5180 | NH2 | ARG | A | 669 | 41.158 | 48.098 | 34.108 | 1.00 | 35.77 |
| ATOM | 5181 | N | TYR | A | 670 | 38.201 | 44.770 | 38.934 | 1.00 | 29.76 |
| ATOM | 5182 | CA | TYR | A | 670 | 38.610 | 43.454 | 39.384 | 1.00 | 30.01 |
| ATOM | 5183 | C | TYR | A | 670 | 38.159 | 43.119 | 40.763 | 1.00 | 30.26 |
| ATOM | 5184 | O | TYR | A | 670 | 38.173 | 41.967 | 41.132 | 1.00 | 31.10 |
| ATOM | 5185 | CB | TYR | A | 670 | 40.132 | 43.310 | 39.314 | 1.00 | 29.59 |
| ATOM | 5186 | CG | TYR | A | 670 | 40.664 | 43.860 | 38.056 | 1.00 | 28.51 |
| ATOM | 5187 | CD1 | TYR | A | 670 | 40.384 | 43.242 | 36.826 | 1.00 | 30.93 |
| ATOM | 5188 | CD2 | TYR | A | 670 | 41.395 | 45.027 | 38.059 | 1.00 | 29.14 |
| ATOM | 5189 | CE1 | TYR | A | 670 | 40.856 | 43.778 | 35.628 | 1.00 | 28.01 |
| ATOM | 5190 | CE2 | TYR | A | 670 | 41.866 | 45.567 | 36.899 | 1.00 | 27.43 |
| ATOM | 5191 | CZ | TYR | A | 670 | 41.615 | 44.927 | 35.704 | 1.00 | 26.81 |
| ATOM | 5192 | OH | TYR | A | 670 | 42.038 | 45.491 | 34.588 | 1.00 | 30.30 |
| ATOM | 5193 | N | MET | A | 671 | 37.804 | 44.098 | 41.576 | 1.00 | 31.12 |
| ATOM | 5194 | CA | MET | A | 671 | 37.517 | 43.742 | 42.969 | 1.00 | 31.05 |
| ATOM | 5195 | C | MET | A | 671 | 36.100 | 44.043 | 43.486 | 1.00 | 31.36 |
| ATOM | 5196 | O | MET | A | 671 | 35.796 | 43.702 | 44.622 | 1.00 | 30.25 |
| ATOM | 5197 | CB | MET | A | 671 | 38.548 | 44.379 | 43.891 | 1.00 | 30.81 |
| ATOM | 5198 | CG | MET | A | 671 | 39.887 | 43.733 | 43.851 | 1.00 | 30.32 |
| ATOM | 5199 | SD | MET | A | 671 | 39.933 | 42.170 | 44.727 | 1.00 | 30.89 |
| ATOM | 5200 | CE | MET | A | 671 | 39.988 | 42.879 | 46.392 | 1.00 | 28.68 |
| ATOM | 5201 | N | GLY | A | 672 | 35.236 | 44.629 | 42.654 | 1.00 | 31.62 |
| ATOM | 5202 | CA | GLY | A | 672 | 33.922 | 45.029 | 43.126 | 1.00 | 32.28 |
| ATOM | 5203 | C | GLY | A | 672 | 34.067 | 46.167 | 44.121 | 1.00 | 33.48 |
| ATOM | 5204 | O | GLY | A | 672 | 35.074 | 46.868 | 44.117 | 1.00 | 34.40 |
| ATOM | 5205 | N | LEU | A | 673 | 33.075 | 46.383 | 44.970 | 1.00 | 33.67 |
| ATOM | 5206 | CA | LEU | A | 673 | 33.175 | 47.451 | 45.946 | 1.00 | 33.93 |
| ATOM | 5207 | C | LEU | A | 673 | 33.612 | 46.919 | 47.304 | 1.00 | 33.21 |
| ATOM | 5208 | O | LEU | A | 673 | 33.271 | 45.802 | 47.667 | 1.00 | 32.81 |
| ATOM | 5209 | CB | LEU | A | 673 | 31.835 | 48.152 | 46.103 | 1.00 | 34.34 |
| ATOM | 5210 | CG | LEU | A | 673 | 31.341 | 49.002 | 44.958 | 1.00 | 35.92 |
| ATOM | 5211 | CD1 | LEU | A | 673 | 29.903 | 49.435 | 45.261 | 1.00 | 38.82 |
| ATOM | 5212 | CD2 | LEU | A | 673 | 32.206 | 50.208 | 44.786 | 1.00 | 36.78 |
| ATOM | 5213 | N | PRO | A | 674 | 34.346 | 47.724 | 48.065 | 1.00 | 32.97 |
| ATOM | 5214 | CA | PRO | A | 674 | 34.777 | 47.323 | 49.399 | 1.00 | 33.06 |
| ATOM | 5215 | C | PRO | A | 674 | 33.721 | 47.625 | 50.497 | 1.00 | 33.83 |
| ATOM | 5216 | O | PRO | A | 674 | 33.996 | 48.429 | 51.402 | 1.00 | 32.53 |
| ATOM | 5217 | CB | PRO | A | 674 | 36.009 | 48.189 | 49.613 | 1.00 | 32.38 |
| ATOM | 5218 | CG | PRO | A | 674 | 35.621 | 49.475 | 49.044 | 1.00 | 32.34 |
| ATOM | 5219 | CD | PRO | A | 674 | 34.826 | 49.083 | 47.758 | 1.00 | 33.10 |
| ATOM | 5220 | N | THR | A | 675 | 32.550 | 46.997 | 50.395 | 1.00 | 34.72 |
| ATOM | 5221 | CA | THR | A | 675 | 31.519 | 47.046 | 51.443 | 1.00 | 36.32 |
| ATOM | 5222 | C | THR | A | 675 | 31.185 | 45.643 | 51.936 | 1.00 | 36.59 |
| ATOM | 5223 | O | THR | A | 675 | 31.431 | 44.661 | 51.258 | 1.00 | 36.02 |
| ATOM | 5224 | CB | THR | A | 675 | 30.208 | 47.605 | 50.910 | 1.00 | 36.75 |
| ATOM | 5225 | OG1 | THR | A | 675 | 29.806 | 46.817 | 49.771 | 1.00 | 38.87 |
| ATOM | 5226 | CG2 | THR | A | 675 | 30.385 | 49.028 | 50.381 | 1.00 | 36.51 |
| ATOM | 5227 | N | PRO | A | 676 | 30.584 | 45.552 | 53.112 | 1.00 | 37.32 |
| ATOM | 5228 | CA | PRO | A | 676 | 30.165 | 44.259 | 53.656 | 1.00 | 37.98 |
| ATOM | 5229 | C | PRO | A | 676 | 29.234 | 43.465 | 52.740 | 1.00 | 38.00 |
| ATOM | 5230 | O | PRO | A | 676 | 29.293 | 42.251 | 52.743 | 1.00 | 37.37 |
| ATOM | 5231 | CB | PRO | A | 676 | 29.451 | 44.655 | 54.952 | 1.00 | 38.17 |
| ATOM | 5232 | CG | PRO | A | 676 | 30.102 | 45.890 | 55.347 | 1.00 | 38.18 |
| ATOM | 5233 | CD | PRO | A | 676 | 30.301 | 46.645 | 54.053 | 1.00 | 37.59 |
| ATOM | 5234 | N | GLU | A | 677 | 28.429 | 44.144 | 51.939 | 1.00 | 39.07 |
| ATOM | 5235 | CA | GLU | A | 677 | 27.490 | 43.463 | 51.060 | 1.00 | 40.38 |
| ATOM | 5236 | C | GLU | A | 677 | 28.139 | 42.998 | 49.751 | 1.00 | 39.46 |
| ATOM | 5237 | O | GLU | A | 677 | 27.626 | 42.096 | 49.104 | 1.00 | 39.65 |
| ATOM | 5238 | CB | GLU | A | 677 | 26.261 | 44.358 | 50.807 | 1.00 | 41.65 |
| ATOM | 5239 | CG | GLU | A | 677 | 26.229 | 45.053 | 49.454 | 1.00 | 45.83 |
| ATOM | 5240 | CD | GLU | A | 677 | 25.316 | 46.285 | 49.437 | 1.00 | 52.13 |
| ATOM | 5241 | OE1 | GLU | A | 677 | 25.569 | 47.207 | 50.256 | 1.00 | 55.30 |
| ATOM | 5242 | OE2 | GLU | A | 677 | 24.365 | 46.341 | 48.596 | 1.00 | 53.85 |
| ATOM | 5243 | N | ASP | A | 678 | 29.275 | 43.591 | 49.366 | 1.00 | 38.46 |
| ATOM | 5244 | CA | ASP | A | 678 | 29.949 | 43.202 | 48.124 | 1.00 | 36.21 |
| ATOM | 5245 | C | ASP | A | 678 | 31.289 | 42.493 | 48.385 | 1.00 | 35.24 |
| ATOM | 5246 | O | ASP | A | 678 | 31.277 | 41.314 | 48.622 | 1.00 | 34.64 |
| ATOM | 5247 | CB | ASP | A | 678 | 30.068 | 44.384 | 47.168 | 1.00 | 36.35 |
| ATOM | 5248 | CG | ASP | A | 678 | 30.594 | 43.979 | 45.787 | 1.00 | 37.08 |
| ATOM | 5249 | OD1 | ASP | A | 678 | 30.891 | 42.770 | 45.598 | 1.00 | 36.62 |
| ATOM | 5250 | OD2 | ASP | A | 678 | 30.739 | 44.794 | 44.834 | 1.00 | 36.77 |
| ATOM | 5251 | N | ASN | A | 679 | 32.445 | 43.156 | 48.378 | 1.00 | 33.79 |
| ATOM | 5252 | CA | ASN | A | 679 | 33.701 | 42.366 | 48.454 | 1.00 | 32.51 |
| ATOM | 5253 | C | ASN | A | 679 | 34.670 | 42.785 | 49.562 | 1.00 | 31.96 |
| ATOM | 5254 | O | ASN | A | 679 | 35.856 | 42.512 | 49.463 | 1.00 | 29.64 |
| ATOM | 5255 | CB | ASN | A | 679 | 34.399 | 42.395 | 47.080 | 1.00 | 32.28 |
| ATOM | 5256 | CG | ASN | A | 679 | 35.400 | 41.251 | 46.849 | 1.00 | 29.98 |
| ATOM | 5257 | OD1 | ASN | A | 679 | 36.394 | 41.448 | 46.157 | 1.00 | 34.09 |
| ATOM | 5258 | ND2 | ASN | A | 679 | 35.141 | 40.077 | 47.383 | 1.00 | 26.34 |
| ATOM | 5259 | N | LEU | A | 680 | 34.163 | 43.385 | 50.649 | 1.00 | 32.19 |
| ATOM | 5260 | CA | LEU | A | 680 | 35.048 | 43.880 | 51.735 | 1.00 | 33.13 |
| ATOM | 5261 | C | LEU | A | 680 | 36.015 | 42.836 | 52.286 | 1.00 | 32.66 |
| ATOM | 5262 | O | LEU | A | 680 | 37.218 | 43.120 | 52.454 | 1.00 | 32.25 |
| ATOM | 5263 | CB | LEU | A | 680 | 34.253 | 44.508 | 52.882 | 1.00 | 33.62 |
| ATOM | 5264 | CG | LEU | A | 680 | 35.024 | 45.198 | 54.019 | 1.00 | 36.92 |
| ATOM | 5265 | CD1 | LEU | A | 680 | 35.911 | 46.349 | 53.515 | 1.00 | 37.94 |
| ATOM | 5266 | CD2 | LEU | A | 680 | 34.020 | 45.718 | 55.110 | 1.00 | 36.39 |
| ATOM | 5267 | N | ASP | A | 681 | 35.513 | 41.633 | 52.553 | 1.00 | 31.71 |
| ATOM | 5268 | CA | ASP | A | 681 | 36.356 | 40.598 | 53.099 | 1.00 | 32.46 |
| ATOM | 5269 | C | ASP | A | 681 | 37.599 | 40.402 | 52.251 | 1.00 | 31.68 |
| ATOM | 5270 | O | ASP | A | 681 | 38.688 | 40.384 | 52.786 | 1.00 | 31.47 |
| ATOM | 5271 | CB | ASP | A | 681 | 35.631 | 39.250 | 53.241 | 1.00 | 33.28 |
| ATOM | 5272 | CG | ASP | A | 681 | 34.621 | 39.226 | 54.411 | 1.00 | 36.99 |
| ATOM | 5273 | OD1 | ASP | A | 681 | 34.514 | 40.224 | 55.155 | 1.00 | 37.92 |
| ATOM | 5274 | OD2 | ASP | A | 681 | 33.899 | 38.220 | 54.659 | 1.00 | 41.78 |
| ATOM | 5275 | N | HIS | A | 682 | 37.461 | 40.231 | 50.945 | 1.00 | 30.30 |
| ATOM | 5276 | CA | HIS | A | 682 | 38.663 | 40.020 | 50.182 | 1.00 | 30.43 |
| ATOM | 5277 | C | HIS | A | 682 | 39.565 | 41.263 | 50.055 | 1.00 | 28.84 |
| ATOM | 5278 | O | HIS | A | 682 | 40.752 | 41.128 | 49.917 | 1.00 | 28.95 |
| ATOM | 5279 | CB | HIS | A | 682 | 38.419 | 39.434 | 48.816 | 1.00 | 30.48 |
| ATOM | 5280 | CG | HIS | A | 682 | 39.704 | 39.091 | 48.132 | 1.00 | 33.22 |
| ATOM | 5281 | ND1 | HIS | A | 682 | 40.619 | 38.219 | 48.684 | 1.00 | 35.26 |
| ATOM | 5282 | CD2 | HIS | A | 682 | 40.277 | 39.570 | 47.004 | 1.00 | 30.92 |
| ATOM | 5283 | CE1 | HIS | A | 682 | 41.672 | 38.128 | 47.897 | 1.00 | 31.98 |
| ATOM | 5284 | NE2 | HIS | A | 682 | 41.496 | 38.950 | 46.880 | 1.00 | 32.30 |
| ATOM | 5285 | N | TYR | A | 683 | 39.012 | 42.456 | 50.053 | 1.00 | 27.68 |
| ATOM | 5286 | CA | TYR | A | 683 | 39.834 | 43.649 | 50.072 | 1.00 | 27.60 |
| ATOM | 5287 | C | TYR | A | 683 | 40.704 | 43.634 | 51.355 | 1.00 | 27.75 |
| ATOM | 5288 | O | TYR | A | 683 | 41.834 | 44.118 | 51.367 | 1.00 | 25.84 |
| ATOM | 5289 | CB | TYR | A | 683 | 38.963 | 44.884 | 50.146 | 1.00 | 26.87 |
| ATOM | 5290 | CG | TYR | A | 683 | 38.554 | 45.591 | 48.850 | 1.00 | 29.98 |
| ATOM | 5291 | CD1 | TYR | A | 683 | 37.402 | 45.230 | 48.157 | 1.00 | 29.80 |
| ATOM | 5292 | CD2 | TYR | A | 683 | 39.283 | 46.691 | 48.382 | 1.00 | 27.64 |
| ATOM | 5293 | CE1 | TYR | A | 683 | 37.000 | 45.939 | 47.005 | 1.00 | 29.62 |
| ATOM | 5294 | CE2 | TYR | A | 683 | 38.900 | 47.373 | 47.278 | 1.00 | 29.75 |
| ATOM | 5295 | CZ | TYR | A | 683 | 37.744 | 47.000 | 46.587 | 1.00 | 29.38 |
| ATOM | 5296 | OH | TYR | A | 683 | 37.389 | 47.704 | 45.476 | 1.00 | 29.36 |
| ATOM | 5297 | N | ARG | A | 684 | 40.179 | 43.056 | 52.433 | 1.00 | 28.35 |
| ATOM | 5298 | CA | ARG | A | 684 | 40.851 | 43.174 | 53.724 | 1.00 | 29.33 |
| ATOM | 5299 | C | ARG | A | 684 | 41.889 | 42.102 | 53.953 | 1.00 | 29.28 |
| ATOM | 5300 | O | ARG | A | 684 | 42.902 | 42.380 | 54.574 | 1.00 | 27.35 |
| ATOM | 5301 | CB | ARG | A | 684 | 39.836 | 43.261 | 54.878 | 1.00 | 30.81 |
| ATOM | 5302 | CG | ARG | A | 684 | 39.623 | 44.659 | 55.354 | 1.00 | 33.87 |
| ATOM | 5303 | CD | ARG | A | 684 | 38.403 | 45.271 | 54.873 | 1.00 | 40.64 |
| ATOM | 5304 | NE | ARG | A | 684 | 38.428 | 46.748 | 54.722 | 1.00 | 45.03 |
| ATOM | 5305 | CZ | ARG | A | 684 | 38.149 | 47.623 | 55.676 | 1.00 | 44.59 |
| ATOM | 5306 | NH1 | ARG | A | 684 | 37.901 | 47.192 | 56.899 | 1.00 | 43.82 |
| ATOM | 5307 | NH2 | ARG | A | 684 | 38.112 | 48.927 | 55.393 | 1.00 | 44.58 |
| ATOM | 5308 | N | ASN | A | 685 | 41.700 | 40.948 | 53.305 | 1.00 | 29.40 |
| ATOM | 5309 | CA | ASN | A | 685 | 42.583 | 39.789 | 53.409 | 1.00 | 30.04 |
| ATOM | 5310 | C | ASN | A | 685 | 43.697 | 39.793 | 52.302 | 1.00 | 28.72 |
| ATOM | 5311 | O | ASN | A | 685 | 44.542 | 38.891 | 52.255 | 1.00 | 27.85 |
| ATOM | 5312 | CB | ASN | A | 685 | 41.724 | 38.491 | 53.340 | 1.00 | 32.20 |
| ATOM | 5313 | CG | ASN | A | 685 | 42.546 | 37.197 | 53.590 | 1.00 | 37.53 |
| ATOM | 5314 | OD1 | ASN | A | 685 | 43.730 | 37.264 | 53.925 | 1.00 | 45.27 |
| ATOM | 5315 | ND2 | ASN | A | 685 | 41.915 | 36.019 | 53.407 | 1.00 | 41.53 |
| ATOM | 5316 | N | SER | A | 686 | 43.723 | 40.825 | 51.456 | 1.00 | 26.15 |
| ATOM | 5317 | CA | SER | A | 686 | 44.703 | 40.884 | 50.362 | 1.00 | 25.93 |
| ATOM | 5318 | C | SER | A | 686 | 45.704 | 42.047 | 50.444 | 1.00 | 25.52 |
| ATOM | 5319 | O | SER | A | 686 | 46.200 | 42.520 | 49.442 | 1.00 | 25.36 |
| ATOM | 5320 | CB | SER | A | 686 | 43.988 | 40.878 | 49.007 | 1.00 | 24.81 |
| ATOM | 5321 | OG | SER | A | 686 | 43.164 | 42.012 | 48.829 | 1.00 | 23.66 |
| ATOM | 5322 | N | THR | A | 687 | 45.990 | 42.490 | 51.657 | 1.00 | 25.47 |
| ATOM | 5323 | CA | THR | A | 687 | 46.950 | 43.544 | 51.883 | 1.00 | 25.57 |
| ATOM | 5324 | C | THR | A | 687 | 48.323 | 42.961 | 52.129 | 1.00 | 24.54 |
| ATOM | 5325 | O | THR | A | 687 | 48.480 | 41.863 | 52.564 | 1.00 | 23.94 |
| ATOM | 5326 | CB | THR | A | 687 | 46.613 | 44.331 | 53.133 | 1.00 | 25.76 |
| ATOM | 5327 | OG1 | THR | A | 687 | 46.899 | 43.506 | 54.242 | 1.00 | 26.17 |
| ATOM | 5328 | CG2 | THR | A | 687 | 45.126 | 44.648 | 53.256 | 1.00 | 26.56 |
| ATOM | 5329 | N | VAL | A | 688 | 49.326 | 43.733 | 51.816 | 1.00 | 25.38 |
| ATOM | 5330 | CA | VAL | A | 688 | 50.688 | 43.335 | 52.102 | 1.00 | 25.28 |
| ATOM | 5331 | C | VAL | A | 688 | 50.865 | 43.423 | 53.615 | 1.00 | 24.05 |
| ATOM | 5332 | O | VAL | A | 688 | 51.516 | 42.599 | 54.224 | 1.00 | 22.44 |
| ATOM | 5333 | CB | VAL | A | 688 | 51.666 | 44.269 | 51.410 | 1.00 | 24.90 |
| ATOM | 5334 | CG1 | VAL | A | 688 | 53.097 | 43.949 | 51.842 | 1.00 | 27.31 |
| ATOM | 5335 | CG2 | VAL | A | 688 | 51.516 | 44.139 | 49.937 | 1.00 | 23.55 |
| ATOM | 5336 | N | MET | A | 689 | 50.222 | 44.395 | 54.229 | 1.00 | 24.40 |
| ATOM | 5337 | CA | MET | A | 689 | 50.450 | 44.604 | 55.655 | 1.00 | 25.35 |
| ATOM | 5338 | C | MET | A | 689 | 50.133 | 43.358 | 56.484 | 1.00 | 26.76 |
| ATOM | 5339 | O | MET | A | 689 | 50.857 | 43.071 | 57.409 | 1.00 | 26.90 |
| ATOM | 5340 | CB | MET | A | 689 | 49.669 | 45.805 | 56.167 | 1.00 | 25.18 |
| ATOM | 5341 | CG | MET | A | 689 | 50.343 | 47.157 | 55.795 | 1.00 | 25.17 |
| ATOM | 5342 | SD | MET | A | 689 | 50.258 | 47.541 | 53.985 | 1.00 | 26.00 |
| ATOM | 5343 | CE | MET | A | 689 | 48.576 | 48.033 | 53.891 | 1.00 | 25.22 |
| ATOM | 5344 | N | SER | A | 690 | 49.120 | 42.576 | 56.120 | 1.00 | 27.17 |
| ATOM | 5345 | CA | SER | A | 690 | 48.743 | 41.471 | 56.984 | 1.00 | 28.78 |
| ATOM | 5346 | C | SER | A | 690 | 49.790 | 40.354 | 57.003 | 1.00 | 28.89 |
| ATOM | 5347 | O | SER | A | 690 | 49.779 | 39.481 | 57.886 | 1.00 | 29.60 |
| ATOM | 5348 | CB | SER | A | 690 | 47.353 | 40.926 | 56.599 | 1.00 | 28.81 |
| ATOM | 5349 | OG | SER | A | 690 | 47.324 | 40.487 | 55.227 | 1.00 | 31.54 |
| ATOM | 5350 | N | ARG | A | 691 | 50.703 | 40.381 | 56.044 | 1.00 | 27.47 |
| ATOM | 5351 | CA | ARG | A | 691 | 51.711 | 39.372 | 55.986 | 1.00 | 27.51 |
| ATOM | 5352 | C | ARG | A | 691 | 53.045 | 39.801 | 56.611 | 1.00 | 27.64 |
| ATOM | 5353 | O | ARG | A | 691 | 54.049 | 39.092 | 56.442 | 1.00 | 27.10 |
| ATOM | 5354 | CB | ARG | A | 691 | 51.876 | 38.942 | 54.528 | 1.00 | 27.22 |
| ATOM | 5355 | CG | ARG | A | 691 | 50.571 | 38.318 | 53.933 | 1.00 | 26.70 |
| ATOM | 5356 | CD | ARG | A | 691 | 50.652 | 38.025 | 52.482 | 1.00 | 29.46 |
| ATOM | 5357 | NE | ARG | A | 691 | 49.423 | 37.469 | 51.911 | 1.00 | 31.01 |
| ATOM | 5358 | CZ | ARG | A | 691 | 49.439 | 36.635 | 50.876 | 1.00 | 31.66 |
| ATOM | 5359 | NH1 | ARG | A | 691 | 50.605 | 36.305 | 50.332 | 1.00 | 28.29 |
| ATOM | 5360 | NN2 | ARG | A | 691 | 48.309 | 36.112 | 50.400 | 1.00 | 32.39 |
| ATOM | 5361 | N | ALA | A | 692 | 53.046 | 40.923 | 57.328 | 1.00 | 27.06 |
| ATOM | 5362 | CA | ALA | A | 692 | 54.290 | 41.505 | 57.849 | 1.00 | 29.54 |
| ATOM | 5363 | C | ALA | A | 692 | 55.258 | 40.552 | 58.496 | 1.00 | 29.91 |
| ATOM | 5364 | O | ALA | A | 692 | 56.439 | 40.580 | 58.189 | 1.00 | 29.93 |
| ATOM | 5365 | CB | ALA | A | 692 | 53.987 | 42.625 | 58.856 | 1.00 | 30.04 |
| ATOM | 5366 | N | GLU | A | 693 | 54.748 | 39.748 | 59.420 | 1.00 | 31.37 |
| ATOM | 5367 | CA | GLU | A | 693 | 55.545 | 38.818 | 60.203 | 1.00 | 32.71 |
| ATOM | 5368 | C | GLU | A | 693 | 56.389 | 37.910 | 59.353 | 1.00 | 32.63 |
| ATOM | 5369 | O | GLU | A | 693 | 57.492 | 37.544 | 59.748 | 1.00 | 31.76 |
| ATOM | 5370 | CB | GLU | A | 693 | 54.639 | 37.898 | 61.025 | 1.00 | 33.70 |
| ATOM | 5371 | CG | GLU | A | 693 | 53.837 | 38.575 | 62.118 | 1.00 | 39.27 |
| ATOM | 5372 | CD | GLU | A | 693 | 54.597 | 38.701 | 63.439 | 1.00 | 44.49 |
| ATOM | 5373 | OE1 | GLU | A | 693 | 55.795 | 38.292 | 63.502 | 1.00 | 42.20 |
| ATOM | 5374 | OE2 | GLU | A | 693 | 53.968 | 39.212 | 64.412 | 1.00 | 46.31 |
| ATOM | 5375 | N | ASN | A | 694 | 55.836 | 37.514 | 58.210 | 1.00 | 32.72 |
| ATOM | 5376 | CA | ASN | A | 694 | 56.511 | 36.606 | 57.307 | 1.00 | 33.40 |
| ATOM | 5377 | C | ASN | A | 694 | 57.767 | 37.238 | 56.690 | 1.00 | 33.58 |
| ATOM | 5378 | O | ASN | A | 694 | 58.667 | 36.534 | 56.298 | 1.00 | 34.60 |
| ATOM | 5379 | CB | ASN | A | 694 | 55.521 | 36.125 | 56.211 | 1.00 | 33.62 |
| ATOM | 5380 | CG | ASN | A | 694 | 54.414 | 35.164 | 56.759 | 1.00 | 35.53 |
| ATOM | 5381 | OD1 | ASN | A | 694 | 54.608 | 34.474 | 57.741 | 1.00 | 40.35 |
| ATOM | 5382 | ND2 | ASN | A | 694 | 53.290 | 35.094 | 56.071 | 1.00 | 40.72 |
| ATOM | 5383 | N | PHE | A | 695 | 57.859 | 38.564 | 56.617 | 1.00 | 32.63 |
| ATOM | 5384 | CA | PHE | A | 695 | 59.011 | 39.160 | 55.992 | 1.00 | 32.04 |
| ATOM | 5385 | C | PHE | A | 695 | 60.322 | 38.897 | 56.779 | 1.00 | 32.80 |
| ATOM | 5386 | O | PHE | A | 695 | 61.408 | 39.201 | 56.341 | 1.00 | 31.80 |
| ATOM | 5387 | CB | PHE | A | 695 | 58.794 | 40.658 | 55.773 | 1.00 | 31.84 |
| ATOM | 5388 | CG | PHE | A | 695 | 57.918 | 40.999 | 54.580 | 1.00 | 30.01 |
| ATOM | 5389 | CD1 | PHE | A | 695 | 56.550 | 40.781 | 54.612 | 1.00 | 28.42 |
| ATOM | 5390 | CD2 | PHE | A | 695 | 58.471 | 41.591 | 53.464 | 1.00 | 32.11 |
| ATOM | 5391 | CE1 | PHE | A | 695 | 55.755 | 41.092 | 53.564 | 1.00 | 27.96 |
| ATOM | 5392 | CE2 | PHE | A | 695 | 57.691 | 41.914 | 52.373 | 1.00 | 33.00 |
| ATOM | 5393 | CZ | PHE | A | 695 | 56.320 | 41.688 | 52.424 | 1.00 | 30.28 |
| ATOM | 5394 | N | LYS | A | 696 | 60.233 | 38.283 | 57.933 | 1.00 | 34.34 |
| ATOM | 5395 | CA | LYS | A | 696 | 61.440 | 38.049 | 58.709 | 1.00 | 35.39 |
| ATOM | 5396 | C | LYS | A | 696 | 62.275 | 36.945 | 58.093 | 1.00 | 35.11 |
| ATOM | 5397 | O | LYS | A | 696 | 63.409 | 36.718 | 58.478 | 1.00 | 35.26 |
| ATOM | 5398 | CB | LYS | A | 696 | 61.053 | 37.670 | 60.127 | 1.00 | 36.41 |
| ATOM | 5399 | CG | LYS | A | 696 | 60.241 | 36.403 | 60.206 | 1.00 | 39.12 |
| ATOM | 5400 | CD | LYS | A | 696 | 59.597 | 36.216 | 61.622 | 1.00 | 44.25 |
| ATOM | 5401 | CE | LYS | A | 696 | 58.616 | 35.023 | 61.628 | 1.00 | 45.71 |
| ATOM | 5402 | NZ | LYS | A | 696 | 57.871 | 34.854 | 62.947 | 1.00 | 47.23 |
| ATOM | 5403 | N | GLN | A | 697 | 61.708 | 36.254 | 57.125 | 1.00 | 34.71 |
| ATOM | 5404 | CA | GLN | A | 697 | 62.380 | 35.138 | 56.499 | 1.00 | 35.52 |
| ATOM | 5405 | C | GLN | A | 697 | 63.187 | 35.522 | 55.295 | 1.00 | 34.21 |
| ATOM | 5406 | O | GLN | A | 697 | 63.848 | 34.677 | 54.731 | 1.00 | 35.13 |
| ATOM | 5407 | CB | GLN | A | 697 | 61.331 | 34.121 | 56.029 | 1.00 | 36.66 |
| ATOM | 5408 | CG | GLN | A | 697 | 60.840 | 33.188 | 57.117 | 1.00 | 40.42 |
| ATOM | 5409 | CD | GLN | A | 697 | 59.659 | 32.367 | 56.642 | 1.00 | 44.24 |
| ATOM | 5410 | OE1 | GLN | A | 697 | 59.817 | 31.445 | 55.803 | 1.00 | 48.10 |
| ATOM | 5411 | NE2 | GLN | A | 697 | 58.483 | 32.693 | 57.148 | 1.00 | 41.55 |
| ATOM | 5412 | N | VAL | A | 698 | 63.112 | 36.782 | 54.894 | 1.00 | 32.68 |
| ATOM | 5413 | CA | VAL | A | 698 | 63.728 | 37.230 | 53.655 | 1.00 | 31.46 |
| ATOM | 5414 | C | VAL | A | 698 | 64.325 | 38.649 | 53.776 | 1.00 | 30.29 |
| ATOM | 5415 | O | VAL | A | 698 | 64.119 | 39.323 | 54.753 | 1.00 | 29.83 |
| ATOM | 5416 | CB | VAL | A | 698 | 62.672 | 37.317 | 52.570 | 1.00 | 31.46 |
| ATOM | 5417 | CG1 | VAL | A | 698 | 61.821 | 36.048 | 52.487 | 1.00 | 28.93 |
| ATOM | 5418 | CG2 | VAL | A | 698 | 61.765 | 38.557 | 52.831 | 1.00 | 30.70 |
| ATOM | 5419 | N | GLU | A | 699 | 65.062 | 39.057 | 52.767 | 1.00 | 28.97 |
| ATOM | 5420 | CA | GLU | A | 699 | 65.604 | 40.415 | 52.648 | 1.00 | 29.28 |
| ATOM | 5421 | C | GLU | A | 699 | 64.813 | 41.169 | 51.591 | 1.00 | 27.24 |
| ATOM | 5422 | O | GLU | A | 699 | 64.712 | 40.710 | 50.445 | 1.00 | 27.18 |
| ATOM | 5423 | CB | GLU | A | 699 | 67.052 | 40.349 | 52.234 | 1.00 | 29.36 |
| ATOM | 5424 | CG | GLU | A | 699 | 67.823 | 39.373 | 53.122 | 1.00 | 33.38 |
| ATOM | 5425 | CD | GLU | A | 699 | 69.169 | 39.010 | 52.541 | 1.00 | 40.83 |
| ATOM | 5426 | OE1 | GLU | A | 699 | 69.909 | 39.934 | 52.077 | 1.00 | 42.56 |
| ATOM | 5427 | OE2 | GLU | A | 699 | 69.462 | 37.785 | 52.524 | 1.00 | 46.50 |
| ATOM | 5428 | N | TYR | A | 700 | 64.288 | 42.321 | 51.965 | 1.00 | 25.29 |
| ATOM | 5429 | CA | TYR | A | 700 | 63.348 | 43.087 | 51.117 | 1.00 | 24.48 |
| ATOM | 5430 | C | TYR | A | 700 | 63.823 | 44.489 | 50.917 | 1.00 | 23.48 |
| ATOM | 5431 | O | TYR | A | 700 | 64.304 | 45.101 | 51.843 | 1.00 | 21.89 |
| ATOM | 5432 | CB | TYR | A | 700 | 62.045 | 43.186 | 51.903 | 1.00 | 24.85 |
| ATOM | 5433 | CG | TYR | A | 700 | 60.811 | 43.889 | 51.344 | 1.00 | 24.00 |
| ATOM | 5434 | CD1 | TYR | A | 700 | 60.348 | 43.655 | 50.069 | 1.00 | 24.47 |
| ATOM | 5435 | CD2 | TYR | A | 700 | 60.002 | 44.629 | 52.201 | 1.00 | 24.32 |
| ATOM | 5436 | CE1 | TYR | A | 700 | 59.153 | 44.248 | 49.606 | 1.00 | 25.02 |
| ATOM | 5437 | CE2 | TYR | A | 700 | 58.818 | 45.204 | 51.767 | 1.00 | 26.02 |
| ATOM | 5438 | CZ | TYR | A | 700 | 58.383 | 45.004 | 50.467 | 1.00 | 25.69 |
| ATOM | 5439 | OH | TYR | A | 700 | 57.190 | 45.585 | 50.088 | 1.00 | 22.93 |
| ATOM | 5440 | N | LEU | A | 701 | 63.647 | 45.017 | 49.725 | 1.00 | 22.74 |
| ATOM | 5441 | CA | LEU | A | 701 | 63.969 | 46.408 | 49.458 | 1.00 | 22.82 |
| ATOM | 5442 | C | LEU | A | 701 | 62.708 | 47.006 | 48.890 | 1.00 | 23.35 |
| ATOM | 5443 | O | LEU | A | 701 | 62.166 | 46.520 | 47.892 | 1.00 | 23.55 |
| ATOM | 5444 | CB | LEU | A | 701 | 65.118 | 46.490 | 48.462 | 1.00 | 22.91 |
| ATOM | 5445 | CG | LEU | A | 701 | 65.497 | 47.829 | 47.856 | 1.00 | 22.89 |
| ATOM | 5446 | CD1 | LEU | A | 701 | 65.913 | 48.884 | 48.907 | 1.00 | 24.19 |
| ATOM | 5447 | CD2 | LEU | A | 701 | 66.608 | 47.557 | 46.920 | 1.00 | 22.79 |
| ATOM | 5448 | N | LEU | A | 702 | 62.251 | 48.073 | 49.531 | 1.00 | 23.76 |
| ATOM | 5449 | CA | LEU | A | 702 | 61.010 | 48.759 | 49.184 | 1.00 | 22.51 |
| ATOM | 5450 | C | LEU | A | 702 | 61.392 | 50.145 | 48.715 | 1.00 | 21.85 |
| ATOM | 5451 | O | LEU | A | 702 | 62.171 | 50.862 | 49.382 | 1.00 | 19.94 |
| ATOM | 5452 | CB | LEU | A | 702 | 60.135 | 48.847 | 50.464 | 1.00 | 23.03 |
| ATOM | 5453 | CG | LEU | A | 702 | 58.799 | 49.601 | 50.342 | 1.00 | 21.66 |
| ATOM | 5454 | CD1 | LEU | A | 702 | 57.813 | 48.855 | 49.423 | 1.00 | 21.68 |
| ATOM | 5455 | CD2 | LEU | A | 702 | 58.164 | 49.820 | 51.680 | 1.00 | 22.17 |
| ATOM | 5456 | N | ILE | A | 703 | 60.866 | 50.535 | 47.561 | 1.00 | 20.85 |
| ATOM | 5457 | CA | ILE | A | 703 | 61.237 | 51.802 | 46.963 | 1.00 | 20.96 |
| ATOM | 5458 | C | ILE | A | 703 | 59.977 | 52.489 | 46.456 | 1.00 | 21.27 |
| ATOM | 5459 | O | ILE | A | 703 | 59.062 | 51.822 | 45.904 | 1.00 | 21.42 |
| ATOM | 5460 | CB | ILE | A | 703 | 62.205 | 51.531 | 45.764 | 1.00 | 21.36 |
| ATOM | 5461 | CG1 | ILE | A | 703 | 63.374 | 50.669 | 46.183 | 1.00 | 20.77 |
| ATOM | 5462 | CG2 | ILE | A | 703 | 62.658 | 52.797 | 45.136 | 1.00 | 21.33 |
| ATOM | 5463 | CD1 | ILE | A | 703 | 64.345 | 50.290 | 45.094 | 1.00 | 22.08 |
| ATOM | 5464 | N | HIS | A | 704 | 59.918 | 53.802 | 46.592 | 1.00 | 20.79 |
| ATOM | 5465 | CA | HIS | A | 704 | 58.737 | 54.539 | 46.133 | 1.00 | 21.54 |
| ATOM | 5466 | C | HIS | A | 704 | 59.070 | 56.017 | 45.954 | 1.00 | 21.73 |
| ATOM | 5467 | O | HIS | A | 704 | 59.865 | 56.581 | 46.696 | 1.00 | 22.47 |
| ATOM | 5468 | CB | HIS | A | 704 | 57.620 | 54.357 | 47.174 | 1.00 | 21.36 |
| ATOM | 5469 | CG | HIS | A | 704 | 56.234 | 54.364 | 46.613 | 1.00 | 22.52 |
| ATOM | 5470 | ND1 | HIS | A | 704 | 55.313 | 53.380 | 46.901 | 1.00 | 20.34 |
| ATOM | 5471 | CD2 | HIS | A | 704 | 55.586 | 55.271 | 45.843 | 1.00 | 22.80 |
| ATOM | 5472 | CE1 | HIS | A | 704 | 54.184 | 53.649 | 46.274 | 1.00 | 22.86 |
| ATOM | 5473 | NE2 | HIS | A | 704 | 54.313 | 54.807 | 45.655 | 1.00 | 20.42 |
| ATOM | 5474 | N | GLY | A | 705 | 58.486 | 56.652 | 44.950 | 1.00 | 22.31 |
| ATOM | 5475 | CA | GLY | A | 705 | 58.654 | 58.077 | 44.775 | 1.00 | 21.36 |
| ATOM | 5476 | C | GLY | A | 705 | 57.634 | 58.815 | 45.596 | 1.00 | 21.59 |
| ATOM | 5477 | O | GLY | A | 705 | 56.461 | 58.390 | 45.698 | 1.00 | 21.44 |
| ATOM | 5478 | N | THR | A | 706 | 58.032 | 59.957 | 46.133 | 1.00 | 21.99 |
| ATOM | 5479 | CA | THR | A | 706 | 57.175 | 60.666 | 47.055 | 1.00 | 22.11 |
| ATOM | 5480 | C | THR | A | 706 | 56.129 | 61.449 | 46.345 | 1.00 | 22.60 |
| ATOM | 5481 | O | THR | A | 706 | 55.177 | 61.844 | 46.967 | 1.00 | 23.64 |
| ATOM | 5482 | CB | THR | A | 706 | 57.985 | 61.602 | 47.999 | 1.00 | 22.70 |
| ATOM | 5483 | OG1 | THR | A | 706 | 58.616 | 62.657 | 47.267 | 1.00 | 21.83 |
| ATOM | 5484 | CG2 | THR | A | 706 | 59.134 | 60.832 | 48.685 | 1.00 | 22.51 |
| ATOM | 5485 | N | ALA | A | 707 | 56.313 | 61.730 | 45.071 | 1.00 | 22.16 |
| ATOM | 5486 | CA | ALA | A | 707 | 55.277 | 62.453 | 44.344 | 1.00 | 23.18 |
| ATOM | 5487 | C | ALA | A | 707 | 54.522 | 61.557 | 43.388 | 1.00 | 22.84 |
| ATOM | 5488 | O | ALA | A | 707 | 54.086 | 62.027 | 42.317 | 1.00 | 22.90 |
| ATOM | 5489 | CB | ALA | A | 707 | 55.868 | 63.635 | 43.565 | 1.00 | 23.66 |
| ATOM | 5490 | N | ASP | A | 708 | 54.366 | 60.285 | 43.758 | 1.00 | 22.28 |
| ATOM | 5491 | CA | ASP | A | 708 | 53.624 | 59.337 | 42.932 | 1.00 | 23.26 |
| ATOM | 5492 | C | ASP | A | 708 | 52.100 | 59.638 | 43.031 | 1.00 | 23.76 |
| ATOM | 5493 | O | ASP | A | 708 | 51.484 | 59.426 | 44.041 | 1.00 | 22.55 |
| ATOM | 5494 | CB | ASP | A | 708 | 54.005 | 57.959 | 43.391 | 1.00 | 22.95 |
| ATOM | 5495 | CG | ASP | A | 708 | 53.609 | 56.843 | 42.417 | 1.00 | 23.76 |
| ATOM | 5496 | OD1 | ASP | A | 708 | 52.633 | 57.029 | 41.678 | 1.00 | 22.38 |
| ATOM | 5497 | OD2 | ASP | A | 708 | 54.176 | 55.707 | 42.429 | 1.00 | 18.33 |
| ATOM | 5498 | N | ASP | A | 709 | 51.510 | 60.161 | 41.953 | 1.00 | 24.61 |
| ATOM | 5499 | CA | ASP | A | 709 | 50.113 | 60.572 | 41.942 | 1.00 | 24.71 |
| ATOM | 5500 | C | ASP | A | 709 | 49.204 | 59.426 | 41.547 | 1.00 | 24.28 |
| ATOM | 5501 | O | ASP | A | 709 | 48.003 | 59.573 | 41.549 | 1.00 | 24.39 |
| ATOM | 5502 | CB | ASP | A | 709 | 49.914 | 61.650 | 40.890 | 1.00 | 25.52 |
| ATOM | 5503 | CG | ASP | A | 709 | 50.408 | 61.179 | 39.528 | 1.00 | 25.66 |
| ATOM | 5504 | OD1 | ASP | A | 709 | 51.643 | 61.032 | 39.357 | 1.00 | 25.08 |
| ATOM | 5505 | OD2 | ASP | A | 709 | 49.653 | 60.840 | 38.616 | 1.00 | 25.79 |
| ATOM | 5506 | N | ASN | A | 710 | 49.771 | 58.255 | 41.338 | 1.00 | 24.32 |
| ATOM | 5507 | CA | ASN | A | 710 | 49.010 | 57.125 | 40.826 | 1.00 | 24.34 |
| ATOM | 5508 | C | ASN | A | 710 | 48.946 | 56.073 | 41.930 | 1.00 | 23.77 |
| ATOM | 5509 | O | ASN | A | 710 | 47.907 | 55.862 | 42.499 | 1.00 | 22.19 |
| ATOM | 5510 | CB | ASN | A | 710 | 49.694 | 56.693 | 39.529 | 1.00 | 23.72 |
| ATOM | 5511 | CG | ASN | A | 710 | 49.111 | 55.457 | 38.877 | 1.00 | 25.68 |
| ATOM | 5512 | OD1 | ASN | A | 710 | 49.565 | 55.108 | 37.760 | 1.00 | 28.26 |
| ATOM | 5513 | ND2 | ASN | A | 710 | 48.155 | 54.793 | 39.503 | 1.00 | 20.50 |
| ATOM | 5514 | N | VAL | A | 711 | 50.057 | 55.420 | 42.238 | 1.00 | 23.96 |
| ATOM | 5515 | CA | VAL | A | 711 | 50.087 | 54.473 | 43.350 | 1.00 | 23.22 |
| ATOM | 5516 | C | VAL | A | 711 | 50.699 | 55.297 | 44.466 | 1.00 | 22.74 |
| ATOM | 5517 | O | VAL | A | 711 | 51.873 | 55.501 | 44.452 | 1.00 | 22.80 |
| ATOM | 5518 | CB | VAL | A | 711 | 50.972 | 53.283 | 43.047 | 1.00 | 22.97 |
| ATOM | 5519 | CG1 | VAL | A | 711 | 51.160 | 52.431 | 44.299 | 1.00 | 23.01 |
| ATOM | 5520 | CG2 | VAL | A | 711 | 50.368 | 52.450 | 41.924 | 1.00 | 23.83 |
| ATOM | 5521 | N | HIS | A | 712 | 49.904 | 55.814 | 45.400 | 1.00 | 22.89 |
| ATOM | 5522 | CA | HIS | A | 712 | 50.416 | 56.834 | 46.359 | 1.00 | 21.66 |
| ATOM | 5523 | C | HIS | A | 712 | 51.501 | 56.353 | 47.274 | 1.00 | 21.27 |
| ATOM | 5524 | O | HIS | A | 712 | 51.530 | 55.188 | 47.648 | 1.00 | 21.83 |
| ATOM | 5525 | CB | HIS | A | 712 | 49.277 | 57.418 | 47.149 | 1.00 | 21.88 |
| ATOM | 5526 | CG | HIS | A | 712 | 48.215 | 57.987 | 46.295 | 1.00 | 21.78 |
| ATOM | 5527 | ND1 | HIS | A | 712 | 46.879 | 57.853 | 46.585 | 1.00 | 23.44 |
| ATOM | 5528 | CD2 | HIS | A | 712 | 48.288 | 58.645 | 45.111 | 1.00 | 24.89 |
| ATOM | 5529 | CE1 | HIS | A | 712 | 46.172 | 58.414 | 45.617 | 1.00 | 26.25 |
| ATOM | 5530 | NE2 | HIS | A | 712 | 47.002 | 58.900 | 44.710 | 1.00 | 23.73 |
| ATOM | 5531 | N | PHE | A | 713 | 52.434 | 57.241 | 47.602 | 1.00 | 21.76 |
| ATOM | 5532 | CA | PHE | A | 713 | 53.548 | 56.891 | 48.497 | 1.00 | 21.66 |
| ATOM | 5533 | C | PHE | A | 713 | 52.955 | 56.240 | 49.755 | 1.00 | 21.63 |
| ATOM | 5534 | O | PHE | A | 713 | 53.514 | 55.305 | 50.331 | 1.00 | 21.09 |
| ATOM | 5535 | CB | PHE | A | 713 | 54.376 | 58.127 | 48.822 | 1.00 | 21.67 |
| ATOM | 5536 | CG | PHE | A | 713 | 55.544 | 57.844 | 49.691 | 1.00 | 22.98 |
| ATOM | 5537 | CD1 | PHE | A | 713 | 56.709 | 57.363 | 49.148 | 1.00 | 23.95 |
| ATOM | 5538 | CD2 | PHE | A | 713 | 55.464 | 58.032 | 51.068 | 1.00 | 23.46 |
| ATOM | 5539 | CE1 | PHE | A | 713 | 57.761 | 57.053 | 49.962 | 1.00 | 24.01 |
| ATOM | 5540 | CE2 | PHE | A | 713 | 56.543 | 57.743 | 51.890 | 1.00 | 24.07 |
| ATOM | 5541 | CZ | PHE | A | 713 | 57.680 | 57.269 | 51.347 | 1.00 | 22.72 |
| ATOM | 5542 | N | GLN | A | 714 | 51.801 | 56.747 | 50.153 | 1.00 | 21.89 |
| ATOM | 5543 | CA | GLN | A | 714 | 50.999 | 56.145 | 51.221 | 1.00 | 21.58 |
| ATOM | 5544 | C | GLN | A | 714 | 51.062 | 54.650 | 51.275 | 1.00 | 21.88 |
| ATOM | 5545 | O | GLN | A | 714 | 51.122 | 54.049 | 52.353 | 1.00 | 21.77 |
| ATOM | 5546 | CB | GLN | A | 714 | 49.530 | 56.516 | 50.996 | 1.00 | 22.22 |
| ATOM | 5547 | CG | GLN | A | 714 | 48.521 | 55.708 | 51.846 | 1.00 | 22.39 |
| ATOM | 5548 | CD | GLN | A | 714 | 47.083 | 55.934 | 51.412 | 1.00 | 25.35 |
| ATOM | 5549 | OE1 | GLN | A | 714 | 46.801 | 56.149 | 50.215 | 1.00 | 19.33 |
| ATOM | 5550 | NE2 | GLN | A | 714 | 46.162 | 55.906 | 52.385 | 1.00 | 24.01 |
| ATOM | 5551 | N | GLN | A | 715 | 50.991 | 54.021 | 50.111 | 1.00 | 22.48 |
| ATOM | 5552 | CA | GLN | A | 715 | 50.863 | 52.567 | 50.098 | 1.00 | 22.47 |
| ATOM | 5553 | C | GLN | A | 715 | 52.113 | 51.959 | 50.686 | 1.00 | 21.96 |
| ATOM | 5554 | O | GLN | A | 715 | 52.039 | 51.017 | 51.456 | 1.00 | 21.62 |
| ATOM | 5555 | CB | GLN | A | 715 | 50.590 | 52.006 | 48.671 | 1.00 | 23.33 |
| ATOM | 5556 | CG | GLN | A | 715 | 49.484 | 52.714 | 47.865 | 1.00 | 22.85 |
| ATOM | 5557 | CD | GLN | A | 715 | 48.460 | 51.803 | 47.206 | 1.00 | 23.26 |
| ATOM | 5558 | OE1 | GLN | A | 715 | 47.763 | 52.237 | 46.256 | 1.00 | 26.33 |
| ATOM | 5559 | NE2 | GLN | A | 715 | 48.357 | 50.553 | 47.672 | 1.00 | 21.21 |
| ATOM | 5560 | N | SER | A | 716 | 53.282 | 52.477 | 50.312 | 1.00 | 21.68 |
| ATOM | 5561 | CA | SER | A | 716 | 54.535 | 51.955 | 50.865 | 1.00 | 21.21 |
| ATOM | 5562 | C | SER | A | 716 | 54.790 | 52.436 | 52.288 | 1.00 | 21.13 |
| ATOM | 5563 | O | SER | A | 716 | 55.427 | 51.732 | 53.076 | 1.00 | 21.25 |
| ATOM | 5564 | CB | SER | A | 716 | 55.724 | 52.393 | 50.028 | 1.00 | 20.77 |
| ATOM | 5565 | OG | SER | A | 716 | 55.750 | 51.785 | 48.782 | 1.00 | 22.33 |
| ATOM | 5566 | N | ALA | A | 717 | 54.341 | 53.643 | 52.613 | 1.00 | 20.84 |
| ATOM | 5567 | CA | ALA | A | 717 | 54.434 | 54.129 | 54.003 | 1.00 | 21.57 |
| ATOM | 5568 | C | ALA | A | 717 | 53.702 | 53.210 | 54.988 | 1.00 | 21.28 |
| ATOM | 5569 | O | ALA | A | 717 | 54.114 | 53.054 | 56.120 | 1.00 | 21.81 |
| ATOM | 5570 | CB | ALA | A | 717 | 53.879 | 55.507 | 54.103 | 1.00 | 21.20 |
| ATOM | 5571 | N | GLN | A | 718 | 52.609 | 52.606 | 54.534 | 1.00 | 21.73 |
| ATOM | 5572 | CA | GLN | A | 718 | 51.833 | 51.680 | 55.345 | 1.00 | 20.77 |
| ATOM | 5573 | C | GLN | A | 718 | 52.543 | 50.336 | 55.332 | 1.00 | 22.09 |
| ATOM | 5574 | O | GLN | A | 718 | 52.531 | 49.610 | 56.321 | 1.00 | 21.08 |
| ATOM | 5575 | CB | GLN | A | 718 | 50.398 | 51.579 | 54.843 | 1.00 | 20.57 |
| ATOM | 5576 | CG | GLN | A | 718 | 49.534 | 52.822 | 55.036 | 1.00 | 19.42 |
| ATOM | 5577 | CD | GLN | A | 718 | 49.086 | 53.107 | 56.516 | 1.00 | 21.11 |
| ATOM | 5578 | OE1 | GLN | A | 718 | 49.500 | 52.423 | 57.442 | 1.00 | 20.25 |
| ATOM | 5579 | NE2 | GLN | A | 718 | 48.233 | 54.128 | 56.700 | 1.00 | 19.84 |
| ATOM | 5580 | N | ILE | A | 719 | 53.220 | 49.980 | 54.230 | 1.00 | 23.16 |
| ATOM | 5581 | CA | ILE | A | 719 | 54.015 | 48.754 | 54.294 | 1.00 | 23.10 |
| ATOM | 5582 | C | ILE | A | 719 | 55.157 | 48.879 | 55.323 | 1.00 | 23.04 |
| ATOM | 5583 | O | ILE | A | 719 | 55.402 | 47.996 | 56.111 | 1.00 | 21.76 |
| ATOM | 5584 | CB | ILE | A | 719 | 54.618 | 48.352 | 52.972 | 1.00 | 23.18 |
| ATOM | 5585 | CG1 | ILE | A | 719 | 53.513 | 47.935 | 52.000 | 1.00 | 24.68 |
| ATOM | 5586 | CG2 | ILE | A | 719 | 55.536 | 47.183 | 53.202 | 1.00 | 23.59 |
| ATOM | 5587 | CD1 | ILE | A | 719 | 54.013 | 47.409 | 50.705 | 1.00 | 24.80 |
| ATOM | 5588 | N | SER | A | 720 | 55.894 | 49.966 | 55.254 | 1.00 | 23.75 |
| ATOM | 5589 | CA | SER | A | 720 | 57.033 | 50.144 | 56.140 | 1.00 | 23.27 |
| ATOM | 5590 | C | SER | A | 720 | 56.568 | 50.144 | 57.604 | 1.00 | 22.52 |
| ATOM | 5591 | O | SER | A | 720 | 57.156 | 49.515 | 58.470 | 1.00 | 23.39 |
| ATOM | 5592 | CB | SER | A | 720 | 57.801 | 51.421 | 55.759 | 1.00 | 22.10 |
| ATOM | 5593 | OG | SER | A | 720 | 57.097 | 52.588 | 56.142 | 1.00 | 22.44 |
| ATOM | 5594 | N | LYS | A | 721 | 55.476 | 50.818 | 57.878 | 1.00 | 23.57 |
| ATOM | 5595 | CA | LYS | A | 721 | 55.037 | 50.922 | 59.238 | 1.00 | 23.10 |
| ATOM | 5596 | C | LYS | A | 721 | 54.591 | 49.594 | 59.802 | 1.00 | 23.13 |
| ATOM | 5597 | O | LYS | A | 721 | 54.776 | 49.314 | 61.013 | 1.00 | 22.76 |
| ATOM | 5598 | CB | LYS | A | 721 | 53.910 | 51.917 | 59.355 | 1.00 | 24.56 |
| ATOM | 5599 | CG | LYS | A | 721 | 53.364 | 52.005 | 60.767 | 1.00 | 22.88 |
| ATOM | 5600 | CD | LYS | A | 721 | 52.518 | 53.195 | 60.884 | 1.00 | 26.29 |
| ATOM | 5601 | CE | LYS | A | 721 | 51.164 | 52.901 | 60.264 | 1.00 | 28.49 |
| ATOM | 5602 | NZ | LYS | A | 721 | 50.635 | 54.174 | 59.874 | 1.00 | 29.77 |
| ATOM | 5603 | N | ALA | A | 722 | 54.045 | 48.741 | 58.942 | 1.00 | 21.71 |
| ATOM | 5604 | CA | ALA | A | 722 | 53.639 | 47.437 | 59.407 | 1.00 | 21.65 |
| ATOM | 5605 | C | ALA | A | 722 | 54.871 | 46.531 | 59.693 | 1.00 | 21.58 |
| ATOM | 5606 | O | ALA | A | 722 | 54.842 | 45.733 | 60.629 | 1.00 | 21.73 |
| ATOM | 5607 | CB | ALA | A | 722 | 52.657 | 46.800 | 58.432 | 1.00 | 22.32 |
| ATOM | 5608 | N | LEU | A | 723 | 55.942 | 46.652 | 58.922 | 1.00 | 21.13 |
| ATOM | 5609 | CA | LEU | A | 723 | 57.132 | 45.849 | 59.176 | 1.00 | 22.59 |
| ATOM | 5610 | C | LEU | A | 723 | 57.833 | 46.308 | 60.477 | 1.00 | 23.17 |
| ATOM | 5611 | O | LEU | A | 723 | 58.415 | 45.510 | 61.161 | 1.00 | 23.51 |
| ATOM | 5612 | CB | LEU | A | 723 | 58.108 | 45.903 | 57.981 | 1.00 | 22.95 |
| ATOM | 5613 | CG | LEU | A | 723 | 57.581 | 45.424 | 56.608 | 1.00 | 25.06 |
| ATOM | 5614 | CD1 | LEU | A | 723 | 58.559 | 45.715 | 55.470 | 1.00 | 25.60 |
| ATOM | 5615 | CD2 | LEU | A | 723 | 57.321 | 43.963 | 56.630 | 1.00 | 26.11 |
| ATOM | 5616 | N | VAL | A | 724 | 57.749 | 47.605 | 60.778 | 1.00 | 25.38 |
| ATOM | 5617 | CA | VAL | A | 724 | 58.296 | 48.200 | 62.001 | 1.00 | 26.75 |
| ATOM | 5618 | C | VAL | A | 724 | 57.515 | 47.720 | 63.211 | 1.00 | 27.37 |
| ATOM | 5619 | O | VAL | A | 724 | 58.121 | 47.337 | 64.192 | 1.00 | 25.90 |
| ATOM | 5620 | CB | VAL | A | 724 | 58.306 | 49.765 | 61.944 | 1.00 | 26.41 |
| ATOM | 5621 | CG1 | VAL | A | 724 | 58.535 | 50.414 | 63.349 | 1.00 | 26.97 |
| ATOM | 5622 | CG2 | VAL | A | 724 | 59.370 | 50.244 | 60.938 | 1.00 | 27.86 |
| ATOM | 5623 | N | ASP | A | 725 | 56.185 | 47.729 | 63.144 | 1.00 | 29.06 |
| ATOM | 5624 | CA | ASP | A | 725 | 55.358 | 47.253 | 64.271 | 1.00 | 30.17 |
| ATOM | 5625 | C | ASP | A | 725 | 55.558 | 45.775 | 64.575 | 1.00 | 30.11 |
| ATOM | 5626 | O | ASP | A | 725 | 55.224 | 45.321 | 65.672 | 1.00 | 30.77 |
| ATOM | 5627 | CB | ASP | A | 725 | 53.866 | 47.502 | 64.029 | 1.00 | 31.42 |
| ATOM | 5628 | CG | ASP | A | 725 | 53.522 | 48.994 | 63.937 | 1.00 | 34.46 |
| ATOM | 5629 | OD1 | ASP | A | 725 | 54.308 | 49.854 | 64.410 | 1.00 | 36.27 |
| ATOM | 5630 | OD2 | ASP | A | 725 | 52.471 | 49.377 | 63.418 | 1.00 | 36.23 |
| ATOM | 5631 | N | VAL | A | 726 | 56.101 | 45.012 | 63.640 | 1.00 | 29.60 |
| ATOM | 5632 | CA | VAL | A | 726 | 56.341 | 43.590 | 63.901 | 1.00 | 30.28 |
| ATOM | 5633 | C | VAL | A | 726 | 57.861 | 43.262 | 64.099 | 1.00 | 30.42 |
| ATOM | 5634 | O | VAL | A | 726 | 58.249 | 42.117 | 64.275 | 1.00 | 31.01 |
| ATOM | 5635 | CB | VAL | A | 726 | 55.626 | 42.761 | 62.768 | 1.00 | 31.65 |
| ATOM | 5636 | CG1 | VAL | A | 726 | 56.253 | 41.453 | 62.563 | 1.00 | 36.09 |
| ATOM | 5637 | CG2 | VAL | A | 726 | 54.135 | 42.552 | 63.096 | 1.00 | 31.33 |
| ATOM | 5638 | N | GLY | A | 727 | 58.723 | 44.277 | 64.124 | 1.00 | 29.48 |
| ATOM | 5639 | CA | GLY | A | 727 | 60.150 | 44.064 | 64.297 | 1.00 | 28.80 |
| ATOM | 5640 | C | GLY | A | 727 | 60.860 | 43.434 | 63.102 | 1.00 | 28.95 |
| ATOM | 5641 | O | GLY | A | 727 | 61.785 | 42.644 | 63.272 | 1.00 | 26.93 |
| ATOM | 5642 | N | VAL | A | 728 | 60.464 | 43.775 | 61.869 | 1.00 | 29.03 |
| ATOM | 5643 | CA | VAL | A | 728 | 61.109 | 43.125 | 60.737 | 1.00 | 28.73 |
| ATOM | 5644 | C | VAL | A | 728 | 62.058 | 44.099 | 60.160 | 1.00 | 27.94 |
| ATOM | 5645 | O | VAL | A | 728 | 61.684 | 45.196 | 59.883 | 1.00 | 27.99 |
| ATOM | 5646 | CB | VAL | A | 728 | 60.082 | 42.672 | 59.639 | 1.00 | 29.59 |
| ATOM | 5647 | CG1 | VAL | A | 728 | 60.754 | 42.337 | 58.345 | 1.00 | 29.88 |
| ATOM | 5648 | CG2 | VAL | A | 728 | 59.350 | 41.464 | 60.091 | 1.00 | 30.54 |
| ATOM | 5649 | N | ASP | A | 729 | 63.309 | 43.736 | 59.986 | 1.00 | 28.23 |
| ATOM | 5650 | CA | ASP | A | 729 | 64.223 | 44.677 | 59.377 | 1.00 | 29.21 |
| ATOM | 5651 | C | ASP | A | 729 | 64.138 | 44.520 | 57.832 | 1.00 | 29.09 |
| ATOM | 5652 | O | ASP | A | 729 | 63.759 | 43.476 | 57.362 | 1.00 | 29.53 |
| ATOM | 5653 | CB | ASP | A | 729 | 65.622 | 44.432 | 59.885 | 1.00 | 29.68 |
| ATOM | 5654 | CG | ASP | A | 729 | 66.604 | 45.518 | 59.439 | 1.00 | 32.15 |
| ATOM | 5655 | OD1 | ASP | A | 729 | 66.262 | 46.732 | 59.395 | 1.00 | 30.16 |
| ATOM | 5656 | OD2 | ASP | A | 729 | 67.772 | 45.225 | 59.140 | 1.00 | 37.36 |
| ATOM | 5657 | N | PHE | A | 730 | 64.433 | 45.575 | 57.088 | 1.00 | 28.48 |
| ATOM | 5658 | CA | PHE | A | 730 | 64.397 | 45.571 | 55.627 | 1.00 | 28.46 |
| ATOM | 5659 | C | PHE | A | 730 | 65.099 | 46.820 | 55.186 | 1.00 | 28.48 |
| ATOM | 5660 | O | PHE | A | 730 | 65.429 | 47.666 | 56.012 | 1.00 | 28.30 |
| ATOM | 5661 | CB | PHE | A | 730 | 62.965 | 45.646 | 55.103 | 1.00 | 28.89 |
| ATOM | 5662 | CG | PHE | A | 730 | 62.222 | 46.857 | 55.585 | 1.00 | 27.56 |
| ATOM | 5663 | CD1 | PHE | A | 730 | 61.704 | 46.895 | 56.851 | 1.00 | 26.58 |
| ATOM | 5664 | CD2 | PHE | A | 730 | 62.081 | 47.960 | 54.788 | 1.00 | 28.64 |
| ATOM | 5665 | CE1 | PHE | A | 730 | 61.031 | 48.016 | 57.316 | 1.00 | 25.09 |
| ATOM | 5666 | CE2 | PHE | A | 730 | 61.424 | 49.083 | 55.247 | 1.00 | 28.36 |
| ATOM | 5667 | CZ | PHE | A | 730 | 60.895 | 49.098 | 56.528 | 1.00 | 27.46 |
| ATOM | 5668 | N | GLN | A | 731 | 65.298 | 46.966 | 53.889 | 1.00 | 28.36 |
| ATOM | 5669 | CA | GLN | A | 731 | 65.953 | 48.144 | 53.363 | 1.00 | 29.43 |
| ATOM | 5670 | C | GLN | A | 731 | 64.909 | 48.998 | 52.632 | 1.00 | 29.60 |
| ATOM | 5671 | O | GLN | A | 731 | 63.884 | 48.482 | 52.143 | 1.00 | 29.07 |
| ATOM | 5672 | CB | GLN | A | 731 | 67.110 | 47.739 | 52.447 | 1.00 | 29.69 |
| ATOM | 5673 | CG | GLN | A | 731 | 68.266 | 46.944 | 53.180 | 1.00 | 34.65 |
| ATOM | 5674 | CD | GLN | A | 731 | 69.065 | 46.054 | 52.228 | 1.00 | 39.33 |
| ATOM | 5675 | OE1 | GLN | A | 731 | 69.361 | 44.845 | 52.519 | 1.00 | 40.43 |
| ATOM | 5676 | NE2 | GLN | A | 731 | 69.438 | 46.638 | 51.089 | 1.00 | 40.02 |
| ATOM | 5677 | N | ALA | A | 732 | 65.217 | 50.285 | 52.493 | 1.00 | 28.93 |
| ATOM | 5678 | CA | ALA | A | 732 | 64.301 | 51.224 | 51.903 | 1.00 | 28.92 |
| ATOM | 5679 | C | ALA | A | 732 | 64.989 | 52.315 | 51.072 | 1.00 | 28.18 |
| ATOM | 5680 | O | ALA | A | 732 | 66.126 | 52.630 | 51.271 | 1.00 | 28.14 |
| ATOM | 5681 | CB | ALA | A | 732 | 63.538 | 51.875 | 52.975 | 1.00 | 29.05 |
| ATOM | 5682 | N | MET | A | 733 | 64.228 | 52.947 | 50.208 | 1.00 | 26.98 |
| ATOM | 5683 | CA | MET | A | 733 | 64.705 | 54.082 | 49.478 | 1.00 | 26.14 |
| ATOM | 5684 | C | MET | A | 733 | 63.474 | 54.827 | 48.990 | 1.00 | 25.35 |
| ATOM | 5685 | O | MET | A | 733 | 62.614 | 54.253 | 48.296 | 1.00 | 25.39 |
| ATOM | 5686 | CB | MET | A | 733 | 65.527 | 53.651 | 48.288 | 1.00 | 25.92 |
| ATOM | 5687 | CG | MET | A | 733 | 65.990 | 54.829 | 47.411 | 1.00 | 28.55 |
| ATOM | 5688 | SD | MET | A | 733 | 67.202 | 55.830 | 48.257 | 1.00 | 31.06 |
| ATOM | 5689 | CE | MET | A | 733 | 68.354 | 54.472 | 48.738 | 1.00 | 30.06 |
| ATOM | 5690 | N | TRP | A | 734 | 63.368 | 56.086 | 49.369 | 1.00 | 24.30 |
| ATOM | 5691 | CA | TRP | A | 734 | 62.312 | 56.941 | 48.858 | 1.00 | 23.89 |
| ATOM | 5692 | C | TRP | A | 734 | 62.965 | 57.750 | 47.759 | 1.00 | 23.75 |
| ATOM | 5693 | O | TRP | A | 734 | 64.171 | 57.880 | 47.786 | 1.00 | 24.07 |
| ATOM | 5694 | CB | TRP | A | 734 | 61.799 | 57.833 | 49.974 | 1.00 | 23.65 |
| ATOM | 5695 | CG | TRP | A | 734 | 62.719 | 58.977 | 50.358 | 1.00 | 21.94 |
| ATOM | 5696 | CD1 | TRP | A | 734 | 62.863 | 60.139 | 49.699 | 1.00 | 20.95 |
| ATOM | 5697 | CD2 | TRP | A | 734 | 63.542 | 59.079 | 51.523 | 1.00 | 19.59 |
| ATOM | 5698 | NE1 | TRP | A | 734 | 63.763 | 60.954 | 50.351 | 1.00 | 21.98 |
| ATOM | 5699 | CE2 | TRP | A | 734 | 64.177 | 60.328 | 51.485 | 1.00 | 19.47 |
| ATOM | 5700 | CE3 | TRP | A | 734 | 63.808 | 58.243 | 52.602 | 1.00 | 19.81 |
| ATOM | 5701 | CZ2 | TRP | A | 734 | 65.064 | 60.745 | 52.455 | 1.00 | 20.40 |
| ATOM | 5702 | CZ3 | TRP | A | 734 | 64.723 | 58.649 | 53.554 | 1.00 | 19.84 |
| ATOM | 5703 | CH2 | TRP | A | 734 | 65.316 | 59.894 | 53.490 | 1.00 | 21.63 |
| ATOM | 5704 | N | TYR | A | 735 | 62.228 | 58.240 | 46.762 | 1.00 | 23.61 |
| ATOM | 5705 | CA | TYR | A | 735 | 62.867 | 59.074 | 45.731 | 1.00 | 24.11 |
| ATOM | 5706 | C | TYR | A | 735 | 62.082 | 60.358 | 45.708 | 1.00 | 23.54 |
| ATOM | 5707 | O | TYR | A | 735 | 60.917 | 60.392 | 45.252 | 1.00 | 22.73 |
| ATOM | 5708 | CB | TYR | A | 735 | 62.927 | 58.420 | 44.330 | 1.00 | 23.78 |
| ATOM | 5709 | CG | TYR | A | 735 | 64.078 | 57.476 | 44.193 | 1.00 | 23.59 |
| ATOM | 5710 | CD1 | TYR | A | 735 | 65.335 | 57.942 | 43.903 | 1.00 | 23.44 |
| ATOM | 5711 | CD2 | TYR | A | 735 | 63.916 | 56.122 | 44.371 | 1.00 | 23.85 |
| ATOM | 5712 | CE1 | TYR | A | 735 | 66.396 | 57.103 | 43.822 | 1.00 | 22.40 |
| ATOM | 5713 | CE2 | TYR | A | 735 | 64.984 | 55.276 | 44.305 | 1.00 | 26.43 |
| ATOM | 5714 | CZ | TYR | A | 735 | 66.235 | 55.784 | 44.016 | 1.00 | 25.37 |
| ATOM | 5715 | OH | TYR | A | 735 | 67.325 | 54.958 | 43.938 | 1.00 | 24.12 |
| ATOM | 5716 | N | THR | A | 736 | 62.729 | 61.425 | 46.174 | 1.00 | 23.46 |
| ATOM | 5717 | CA | THR | A | 736 | 62.009 | 62.673 | 46.288 | 1.00 | 22.55 |
| ATOM | 5718 | C | THR | A | 736 | 61.636 | 63.356 | 45.010 | 1.00 | 23.03 |
| ATOM | 5719 | O | THR | A | 736 | 62.434 | 63.507 | 44.094 | 1.00 | 22.95 |
| ATOM | 5720 | CB | THR | A | 736 | 62.527 | 63.627 | 47.410 | 1.00 | 23.38 |
| ATOM | 5721 | OG1 | THR | A | 736 | 62.759 | 64.943 | 46.931 | 1.00 | 19.53 |
| ATOM | 5722 | CG2 | THR | A | 736 | 63.748 | 63.165 | 48.096 | 1.00 | 21.12 |
| ATOM | 5723 | N | ASP | A | 737 | 60.341 | 63.678 | 44.976 | 1.00 | 23.42 |
| ATOM | 5724 | CA | ASP | A | 737 | 59.642 | 64.375 | 43.929 | 1.00 | 23.60 |
| ATOM | 5725 | C | ASP | A | 737 | 59.514 | 63.537 | 42.632 | 1.00 | 24.09 |
| ATOM | 5726 | O | ASP | A | 737 | 59.127 | 64.055 | 41.586 | 1.00 | 25.66 |
| ATOM | 5727 | CB | ASP | A | 737 | 60.289 | 65.751 | 43.664 | 1.00 | 23.78 |
| ATOM | 5728 | CG | ASP | A | 737 | 59.901 | 66.832 | 44.699 | 1.00 | 24.93 |
| ATOM | 5729 | OD1 | ASP | A | 737 | 59.103 | 66.578 | 45.666 | 1.00 | 26.08 |
| ATOM | 5730 | OD2 | ASP | A | 737 | 60.392 | 68.008 | 44.638 | 1.00 | 24.36 |
| ATOM | 5731 | N | GLU | A | 738 | 59.825 | 62.259 | 42.696 | 1.00 | 24.70 |
| ATOM | 5732 | CA | GLU | A | 738 | 59.634 | 61.377 | 41.565 | 1.00 | 25.89 |
| ATOM | 5733 | C | GLU | A | 738 | 58.203 | 60.778 | 41.573 | 1.00 | 25.42 |
| ATOM | 5734 | O | GLU | A | 738 | 57.594 | 60.595 | 42.647 | 1.00 | 26.04 |
| ATOM | 5735 | CB | GLU | A | 738 | 60.621 | 60.221 | 41.653 | 1.00 | 26.72 |
| ATOM | 5736 | CG | GLU | A | 738 | 62.029 | 60.501 | 41.164 | 1.00 | 28.86 |
| ATOM | 5737 | CD | GLU | A | 738 | 62.054 | 61.057 | 39.764 | 1.00 | 31.38 |
| ATOM | 5738 | OE1 | GLU | A | 738 | 61.602 | 60.349 | 38.821 | 1.00 | 30.60 |
| ATOM | 5739 | OE2 | GLU | A | 738 | 62.518 | 62.205 | 39.634 | 1.00 | 31.30 |
| ATOM | 5740 | N | ASP | A | 739 | 57.672 | 60.439 | 40.398 | 1.00 | 25.27 |
| ATOM | 5741 | CA | ASP | A | 739 | 56.303 | 59.841 | 40.313 | 1.00 | 24.84 |
| ATOM | 5742 | C | ASP | A | 739 | 56.306 | 58.346 | 40.117 | 1.00 | 23.80 |
| ATOM | 5743 | O | ASP | A | 739 | 57.271 | 57.696 | 40.435 | 1.00 | 24.54 |
| ATOM | 5744 | CB | ASP | A | 739 | 55.440 | 60.535 | 39.267 | 1.00 | 23.94 |
| ATOM | 5745 | CG | ASP | A | 739 | 55.999 | 60.417 | 37.863 | 1.00 | 24.92 |
| ATOM | 5746 | OD1 | ASP | A | 739 | 56.741 | 59.470 | 37.526 | 1.00 | 23.02 |
| ATOM | 5747 | OD2 | ASP | A | 739 | 55.667 | 61.211 | 36.999 | 1.00 | 28.71 |
| ATOM | 5748 | N | HIS | A | 740 | 55.231 | 57.781 | 39.595 | 1.00 | 23.98 |
| ATOM | 5749 | CA | HIS | A | 740 | 55.135 | 56.331 | 39.442 | 1.00 | 23.70 |
| ATOM | 5750 | C | HIS | A | 740 | 56.210 | 55.741 | 38.510 | 1.00 | 23.84 |
| ATOM | 5751 | O | HIS | A | 740 | 56.576 | 54.593 | 38.651 | 1.00 | 23.71 |
| ATOM | 5752 | CB | HIS | A | 740 | 53.772 | 55.959 | 38.866 | 1.00 | 23.70 |
| ATOM | 5753 | CG | HIS | A | 740 | 53.382 | 54.554 | 39.160 | 1.00 | 25.99 |
| ATOM | 5754 | ND1 | HIS | A | 740 | 53.592 | 53.977 | 40.397 | 1.00 | 26.03 |
| ATOM | 5755 | CD2 | HIS | A | 740 | 52.802 | 53.600 | 38.387 | 1.00 | 30.23 |
| ATOM | 5756 | CE1 | HIS | A | 740 | 53.150 | 52.733 | 40.374 | 1.00 | 28.49 |
| ATOM | 5757 | NE2 | HIS | A | 740 | 52.658 | 52.480 | 39.169 | 1.00 | 28.58 |
| ATOM | 5758 | N | GLY | A | 741 | 56.681 | 56.535 | 37.552 | 1.00 | 24.11 |
| ATOM | 5759 | CA | GLY | A | 741 | 57.718 | 56.102 | 36.630 | 1.00 | 24.98 |
| ATOM | 5760 | C | GLY | A | 741 | 59.147 | 56.281 | 37.093 | 1.00 | 25.21 |
| ATOM | 5761 | O | GLY | A | 741 | 60.049 | 55.720 | 36.462 | 1.00 | 27.78 |
| ATOM | 5762 | N | ILE | A | 742 | 59.370 | 57.036 | 38.163 | 1.00 | 24.28 |
| ATOM | 5763 | CA | ILE | A | 742 | 60.705 | 57.304 | 38.668 | 1.00 | 24.91 |
| ATOM | 5764 | C | ILE | A | 742 | 61.654 | 57.350 | 37.466 | 1.00 | 25.71 |
| ATOM | 5765 | O | ILE | A | 742 | 62.585 | 56.603 | 37.420 | 1.00 | 24.94 |
| ATOM | 5766 | CB | ILE | A | 742 | 61.209 | 56.243 | 39.746 | 1.00 | 25.62 |
| ATOM | 5767 | CG1 | ILE | A | 742 | 60.207 | 56.063 | 40.898 | 1.00 | 24.75 |
| ATOM | 5768 | CG2 | ILE | A | 742 | 62.507 | 56.668 | 40.361 | 1.00 | 23.09 |
| ATOM | 5769 | CD1 | ILE | A | 742 | 60.502 | 54.911 | 41.752 | 1.00 | 26.48 |
| ATOM | 5770 | N | ALA | A | 743 | 61.437 | 58.281 | 36.545 | 1.00 | 26.23 |
| ATOM | 5771 | CA | ALA | A | 743 | 62.089 | 58.214 | 35.256 | 1.00 | 27.99 |
| ATOM | 5772 | C | ALA | A | 743 | 62.870 | 59.440 | 34.873 | 1.00 | 28.21 |
| ATOM | 5773 | O | ALA | A | 743 | 63.293 | 59.519 | 33.736 | 1.00 | 28.10 |
| ATOM | 5774 | CB | ALA | A | 743 | 60.999 | 57.924 | 34.117 | 1.00 | 28.61 |
| ATOM | 5775 | N | SER | A | 744 | 63.001 | 60.437 | 35.735 | 1.00 | 27.98 |
| ATOM | 5776 | CA | SER | A | 744 | 63.927 | 61.485 | 35.369 | 1.00 | 29.60 |
| ATOM | 5777 | C | SER | A | 744 | 65.268 | 60.734 | 35.212 | 1.00 | 29.10 |
| ATOM | 5778 | O | SER | A | 744 | 65.435 | 59.690 | 35.841 | 1.00 | 29.17 |
| ATOM | 5779 | CB | SER | A | 744 | 63.909 | 62.637 | 36.405 | 1.00 | 29.43 |
| ATOM | 5780 | OG | SER | A | 744 | 64.575 | 62.278 | 37.565 | 1.00 | 35.33 |
| ATOM | 5781 | N | SER | A | 745 | 66.186 | 61.158 | 34.323 | 1.00 | 29.84 |
| ATOM | 5782 | CA | SER | A | 745 | 67.418 | 60.366 | 34.078 | 1.00 | 29.46 |
| ATOM | 5783 | C | SER | A | 745 | 68.256 | 60.108 | 35.306 | 1.00 | 28.35 |
| ATOM | 5784 | O | SER | A | 745 | 68.708 | 58.991 | 35.484 | 1.00 | 29.11 |
| ATOM | 5785 | CB | SER | A | 745 | 68.320 | 60.991 | 33.000 | 1.00 | 30.81 |
| ATOM | 5786 | OG | SER | A | 745 | 68.189 | 62.390 | 33.082 | 1.00 | 35.58 |
| ATOM | 5787 | N | THR | A | 746 | 68.472 | 61.091 | 36.164 | 1.00 | 27.21 |
| ATOM | 5788 | CA | THR | A | 746 | 69.252 | 60.797 | 37.370 | 1.00 | 27.83 |
| ATOM | 5789 | C | THR | A | 746 | 68.560 | 59.804 | 38.281 | 1.00 | 26.82 |
| ATOM | 5790 | O | THR | A | 746 | 69.204 | 58.902 | 38.767 | 1.00 | 24.88 |
| ATOM | 5791 | CB | THR | A | 746 | 69.584 | 62.021 | 38.176 | 1.00 | 27.88 |
| ATOM | 5792 | OG1 | THR | A | 746 | 68.398 | 62.792 | 38.395 | 1.00 | 29.32 |
| ATOM | 5793 | CG2 | THR | A | 746 | 70.580 | 62.912 | 37.400 | 1.00 | 29.80 |
| ATOM | 5794 | N | ALA | A | 747 | 67.249 | 59.933 | 38.483 | 1.00 | 26.80 |
| ATOM | 5795 | CA | ALA | A | 747 | 66.589 | 58.974 | 39.380 | 1.00 | 26.79 |
| ATOM | 5796 | C | ALA | A | 747 | 66.598 | 57.594 | 38.747 | 1.00 | 26.91 |
| ATOM | 5797 | O | ALA | A | 747 | 66.853 | 56.597 | 39.410 | 1.00 | 28.23 |
| ATOM | 5798 | CB | ALA | A | 747 | 65.199 | 59.398 | 39.692 | 1.00 | 27.13 |
| ATOM | 5799 | N | HIS | A | 748 | 66.319 | 57.513 | 37.451 | 1.00 | 26.90 |
| ATOM | 5800 | CA | HIS | A | 748 | 66.323 | 56.221 | 36.779 | 1.00 | 25.91 |
| ATOM | 5801 | C | HIS | A | 748 | 67.712 | 55.601 | 36.943 | 1.00 | 24.98 |
| ATOM | 5802 | O | HIS | A | 748 | 67.857 | 54.444 | 37.288 | 1.00 | 24.25 |
| ATOM | 5803 | CB | HIS | A | 748 | 65.995 | 56.434 | 35.290 | 1.00 | 26.95 |
| ATOM | 5804 | CG | HIS | A | 748 | 66.175 | 55.220 | 34.452 | 1.00 | 26.97 |
| ATOM | 5805 | ND1 | HIS | A | 748 | 65.215 | 54.252 | 34.345 | 1.00 | 26.07 |
| ATOM | 5806 | CD2 | HIS | A | 748 | 67.198 | 54.829 | 33.660 | 1.00 | 30.08 |
| ATOM | 5807 | CE1 | HIS | A | 748 | 65.644 | 53.299 | 33.540 | 1.00 | 30.19 |
| ATOM | 5808 | NE2 | HIS | A | 748 | 66.852 | 53.620 | 33.119 | 1.00 | 28.24 |
| ATOM | 5809 | N | GLN | A | 749 | 68.758 | 56.384 | 36.748 | 1.00 | 24.83 |
| ATOM | 5810 | CA | GLN | A | 749 | 70.109 | 55.821 | 36.866 | 1.00 | 24.85 |
| ATOM | 5811 | C | GLN | A | 749 | 70.350 | 55.378 | 38.323 | 1.00 | 23.61 |
| ATOM | 5812 | O | GLN | A | 749 | 70.890 | 54.295 | 38.603 | 1.00 | 21.42 |
| ATOM | 5813 | CB | GLN | A | 749 | 71.156 | 56.848 | 36.423 | 1.00 | 25.62 |
| ATOM | 5814 | CG | GLN | A | 749 | 71.047 | 57.212 | 34.965 | 1.00 | 28.72 |
| ATOM | 5815 | CD | GLN | A | 749 | 72.024 | 58.289 | 34.547 | 1.00 | 35.38 |
| ATOM | 5816 | OE1 | GLN | A | 749 | 73.220 | 58.031 | 34.446 | 1.00 | 40.06 |
| ATOM | 5817 | NE2 | GLN | A | 749 | 71.524 | 59.494 | 34.295 | 1.00 | 36.86 |
| ATOM | 5818 | N | HIS | A | 750 | 69.838 | 56.167 | 39.257 | 1.00 | 22.63 |
| ATOM | 5819 | CA | HIS | A | 750 | 70.119 | 55.874 | 40.639 | 1.00 | 24.02 |
| ATOM | 5820 | C | HIS | A | 750 | 69.340 | 54.675 | 41.143 | 1.00 | 22.54 |
| ATOM | 5821 | O | HIS | A | 750 | 69.909 | 53.814 | 41.764 | 1.00 | 23.89 |
| ATOM | 5822 | CB | HIS | A | 750 | 69.966 | 57.148 | 41.492 | 1.00 | 24.83 |
| ATOM | 5823 | CG | HIS | A | 750 | 70.304 | 56.957 | 42.934 | 1.00 | 27.84 |
| ATOM | 5824 | ND1 | HIS | A | 750 | 71.263 | 57.705 | 43.572 | 1.00 | 30.28 |
| ATOM | 5825 | CD2 | HIS | A | 750 | 69.812 | 56.104 | 43.863 | 1.00 | 28.68 |
| ATOM | 5826 | CE1 | HIS | A | 750 | 71.343 | 57.332 | 44.837 | 1.00 | 27.63 |
| ATOM | 5827 | NE2 | HIS | A | 750 | 70.485 | 56.348 | 45.034 | 1.00 | 28.75 |
| ATOM | 5828 | N | ILE | A | 751 | 68.073 | 54.535 | 40.831 | 1.00 | 23.23 |
| ATOM | 5829 | CA | ILE | A | 751 | 67.355 | 53.328 | 41.292 | 1.00 | 22.24 |
| ATOM | 5830 | C | ILE | A | 751 | 67.920 | 52.028 | 40.729 | 1.00 | 23.39 |
| ATOM | 5831 | O | ILE | A | 751 | 68.004 | 51.037 | 41.442 | 1.00 | 23.47 |
| ATOM | 5832 | CB | ILE | A | 751 | 65.847 | 53.429 | 41.013 | 1.00 | 21.86 |
| ATOM | 5833 | CG1 | ILE | A | 751 | 65.057 | 52.351 | 41.771 | 1.00 | 21.82 |
| ATOM | 5834 | CG2 | ILE | A | 751 | 65.526 | 53.269 | 39.575 | 1.00 | 21.39 |
| ATOM | 5835 | CD1 | ILE | A | 751 | 63.490 | 52.447 | 41.548 | 1.00 | 22.67 |
| ATOM | 5836 | N | TYR | A | 752 | 68.256 | 51.981 | 39.439 | 1.00 | 23.82 |
| ATOM | 5837 | CA | TYR | A | 752 | 68.771 | 50.716 | 38.890 | 1.00 | 24.50 |
| ATOM | 5838 | C | TYR | A | 752 | 70.154 | 50.417 | 39.436 | 1.00 | 23.94 |
| ATOM | 5839 | O | TYR | A | 752 | 70.538 | 49.276 | 39.554 | 1.00 | 24.99 |
| ATOM | 5840 | CB | TYR | A | 752 | 68.731 | 50.678 | 37.353 | 1.00 | 24.24 |
| ATOM | 5841 | CG | TYR | A | 752 | 67.348 | 50.335 | 36.850 | 1.00 | 24.42 |
| ATOM | 5842 | CD1 | TYR | A | 752 | 66.895 | 49.021 | 36.863 | 1.00 | 25.36 |
| ATOM | 5843 | CD2 | TYR | A | 752 | 66.491 | 51.310 | 36.418 | 1.00 | 23.66 |
| ATOM | 5844 | CE1 | TYR | A | 752 | 65.662 | 48.698 | 36.388 | 1.00 | 26.62 |
| ATOM | 5845 | CE2 | TYR | A | 752 | 65.230 | 50.996 | 35.993 | 1.00 | 26.62 |
| ATOM | 5846 | CZ | TYR | A | 752 | 64.823 | 49.679 | 35.983 | 1.00 | 27.74 |
| ATOM | 5847 | OH | TYR | A | 752 | 63.552 | 49.341 | 35.576 | 1.00 | 31.07 |
| ATOM | 5848 | N | THR | A | 753 | 70.881 | 51.445 | 39.792 | 1.00 | 23.66 |
| ATOM | 5849 | CA | THR | A | 753 | 72.180 | 51.266 | 40.416 | 1.00 | 24.64 |
| ATOM | 5850 | C | THR | A | 753 | 72.003 | 50.689 | 41.809 | 1.00 | 24.38 |
| ATOM | 5851 | O | THR | A | 753 | 72.634 | 49.706 | 42.159 | 1.00 | 24.12 |
| ATOM | 5852 | CB | THR | A | 753 | 72.948 | 52.620 | 40.431 | 1.00 | 25.26 |
| ATOM | 5853 | OG1 | THR | A | 753 | 73.155 | 53.053 | 39.068 | 1.00 | 25.67 |
| ATOM | 5854 | CG2 | THR | A | 753 | 74.346 | 52.468 | 40.989 | 1.00 | 24.69 |
| ATOM | 5855 | N | HIS | A | 754 | 71.066 | 51.235 | 42.571 | 1.00 | 25.36 |
| ATOM | 5856 | CA | HIS | A | 754 | 70.832 | 50.753 | 43.940 | 1.00 | 25.62 |
| ATOM | 5857 | C | HIS | A | 754 | 70.298 | 49.342 | 43.886 | 1.00 | 25.39 |
| ATOM | 5858 | O | HIS | A | 754 | 70.694 | 48.478 | 44.673 | 1.00 | 24.87 |
| ATOM | 5859 | CB | HIS | A | 754 | 69.855 | 51.667 | 44.667 | 1.00 | 26.41 |
| ATOM | 5860 | CG | HIS | A | 754 | 69.948 | 51.631 | 46.158 | 1.00 | 27.10 |
| ATOM | 5861 | ND1 | HIS | A | 754 | 71.102 | 51.933 | 46.843 | 1.00 | 30.98 |
| ATOM | 5862 | CD2 | HIS | A | 754 | 69.012 | 51.349 | 47.101 | 1.00 | 29.11 |
| ATOM | 5863 | CE1 | HIS | A | 754 | 70.877 | 51.807 | 48.146 | 1.00 | 32.00 |
| ATOM | 5864 | NE2 | HIS | A | 754 | 69.620 | 51.443 | 48.320 | 1.00 | 27.79 |
| ATOM | 5865 | N | MET | A | 755 | 69.380 | 49.086 | 42.971 | 1.00 | 24.38 |
| ATOM | 5866 | CA | MET | A | 755 | 68.807 | 47.753 | 42.904 | 1.00 | 24.59 |
| ATOM | 5867 | C | MET | A | 755 | 69.860 | 46.718 | 42.458 | 1.00 | 24.66 |
| ATOM | 5868 | O | MET | A | 755 | 69.820 | 45.559 | 42.880 | 1.00 | 23.92 |
| ATOM | 5869 | CB | MET | A | 755 | 67.606 | 47.727 | 41.968 | 1.00 | 24.88 |
| ATOM | 5870 | CG | MET | A | 755 | 66.364 | 48.479 | 42.427 | 1.00 | 26.01 |
| ATOM | 5871 | SD | MET | A | 755 | 64.919 | 48.067 | 41.396 | 1.00 | 28.08 |
| ATOM | 5872 | CE | MET | A | 755 | 65.463 | 48.670 | 39.959 | 1.00 | 29.78 |
| ATOM | 5873 | N | SER | A | 756 | 70.809 | 47.118 | 41.612 | 1.00 | 25.65 |
| ATOM | 5874 | CA | SER | A | 756 | 71.831 | 46.155 | 41.172 | 1.00 | 26.67 |
| ATOM | 5875 | C | SER | A | 756 | 72.724 | 45.760 | 42.352 | 1.00 | 26.83 |
| ATOM | 5876 | O | SER | A | 756 | 72.988 | 44.568 | 42.559 | 1.00 | 26.03 |
| ATOM | 5877 | CB | SER | A | 756 | 72.701 | 46.723 | 40.043 | 1.00 | 26.64 |
| ATOM | 5878 | OG | SER | A | 756 | 71.911 | 47.057 | 38.918 | 1.00 | 28.16 |
| ATOM | 5879 | N | HIS | A | 757 | 73.171 | 46.750 | 43.139 | 1.00 | 27.24 |
| ATOM | 5880 | CA | HIS | A | 757 | 73.969 | 46.437 | 44.313 | 1.00 | 27.68 |
| ATOM | 5881 | C | HIS | A | 757 | 73.222 | 45.445 | 45.171 | 1.00 | 27.98 |
| ATOM | 5882 | O | HIS | A | 757 | 73.808 | 44.452 | 45.642 | 1.00 | 28.11 |
| ATOM | 5883 | CB | HIS | A | 757 | 74.315 | 47.685 | 45.147 | 1.00 | 28.80 |
| ATOM | 5884 | CG | HIS | A | 757 | 75.311 | 48.605 | 44.489 | 1.00 | 29.61 |
| ATOM | 5885 | ND1 | HIS | A | 757 | 76.438 | 48.146 | 43.838 | 1.00 | 34.38 |
| ATOM | 5886 | CD2 | HIS | A | 757 | 75.363 | 49.956 | 44.418 | 1.00 | 30.86 |
| ATOM | 5887 | CE1 | HIS | A | 757 | 77.124 | 49.177 | 43.370 | 1.00 | 36.17 |
| ATOM | 5888 | NE2 | HIS | A | 757 | 76.493 | 50.289 | 43.715 | 1.00 | 33.47 |
| ATOM | 5889 | N | PHE | A | 758 | 71.915 | 45.649 | 45.335 | 1.00 | 27.26 |
| ATOM | 5890 | CA | PHE | A | 758 | 71.140 | 44.810 | 46.240 | 1.00 | 26.79 |
| ATOM | 5891 | C | PHE | A | 758 | 71.021 | 43.381 | 45.735 | 1.00 | 28.64 |
| ATOM | 5892 | O | PHE | A | 758 | 71.187 | 42.438 | 46.508 | 1.00 | 28.71 |
| ATOM | 5893 | CB | PHE | A | 758 | 69.771 | 45.447 | 46.514 | 1.00 | 26.31 |
| ATOM | 5894 | CG | PHE | A | 758 | 68.874 | 44.611 | 47.374 | 1.00 | 25.47 |
| ATOM | 5895 | CD1 | PHE | A | 758 | 68.008 | 43.691 | 46.801 | 1.00 | 24.16 |
| ATOM | 5896 | CD2 | PHE | A | 758 | 68.868 | 44.753 | 48.753 | 1.00 | 24.21 |
| ATOM | 5897 | CE1 | PHE | A | 758 | 67.143 | 42.914 | 47.595 | 1.00 | 23.28 |
| ATOM | 5898 | CE2 | PHE | A | 758 | 68.015 | 43.957 | 49.547 | 1.00 | 25.66 |
| ATOM | 5899 | CZ | PHE | A | 758 | 67.164 | 43.030 | 48.943 | 1.00 | 25.26 |
| ATOM | 5900 | N | ILE | A | 759 | 70.771 | 43.186 | 44.442 | 1.00 | 29.59 |
| ATOM | 5901 | CA | ILE | A | 759 | 70.711 | 41.827 | 43.920 | 1.00 | 30.59 |
| ATOM | 5902 | C | ILE | A | 759 | 72.091 | 41.168 | 44.009 | 1.00 | 31.21 |
| ATOM | 5903 | O | ILE | A | 759 | 72.204 | 39.986 | 44.386 | 1.00 | 30.69 |
| ATOM | 5904 | CB | ILE | A | 759 | 70.215 | 41.816 | 42.465 | 1.00 | 31.21 |
| ATOM | 5905 | CG1 | ILE | A | 759 | 68.740 | 42.175 | 42.397 | 1.00 | 33.66 |
| ATOM | 5906 | CG2 | ILE | A | 759 | 70.465 | 40.451 | 41.860 | 1.00 | 33.00 |
| ATOM | 5907 | CD1 | ILE | A | 759 | 67.781 | 41.070 | 43.005 | 1.00 | 35.66 |
| ATOM | 5908 | N | LYS | A | 760 | 73.140 | 41.922 | 43.668 | 1.00 | 32.46 |
| ATOM | 5909 | CA | LYS | A | 760 | 74.514 | 41.392 | 43.708 | 1.00 | 33.72 |
| ATOM | 5910 | C | LYS | A | 760 | 74.896 | 40.871 | 45.109 | 1.00 | 34.58 |
| ATOM | 5911 | O | LYS | A | 760 | 75.415 | 39.770 | 45.248 | 1.00 | 35.10 |
| ATOM | 5912 | CB | LYS | A | 760 | 75.523 | 42.433 | 43.174 | 1.00 | 33.78 |
| ATOM | 5913 | CG | LYS | A | 760 | 75.359 | 42.680 | 41.645 | 1.00 | 35.41 |
| ATOM | 5914 | CD | LYS | A | 760 | 76.636 | 42.811 | 40.894 | 1.00 | 37.74 |
| ATOM | 5915 | CE | LYS | A | 760 | 77.512 | 43.919 | 41.411 | 1.00 | 39.01 |
| ATOM | 5916 | NZ | LYS | A | 760 | 78.711 | 44.128 | 40.596 | 1.00 | 39.64 |
| ATOM | 5917 | N | GLN | A | 761 | 74.573 | 41.612 | 46.160 | 1.00 | 35.44 |
| ATOM | 5918 | CA | GLN | A | 761 | 74.928 | 41.152 | 47.495 | 1.00 | 35.72 |
| ATOM | 5919 | C | GLN | A | 761 | 74.048 | 40.003 | 47.970 | 1.00 | 36.14 |
| ATOM | 5920 | O | GLN | A | 761 | 74.552 | 39.051 | 48.584 | 1.00 | 34.84 |
| ATOM | 5921 | CB | GLN | A | 761 | 74.992 | 42.308 | 48.476 | 1.00 | 36.42 |
| ATOM | 5922 | CG | GLN | A | 761 | 73.719 | 42.855 | 48.947 | 1.00 | 39.46 |
| ATOM | 5923 | CD | GLN | A | 761 | 73.014 | 41.956 | 49.923 | 1.00 | 44.27 |
| ATOM | 5924 | OE1 | GLN | A | 761 | 73.652 | 41.146 | 50.597 | 1.00 | 47.15 |
| ATOM | 5925 | NE2 | GLN | A | 761 | 71.669 | 42.072 | 49.987 | 1.00 | 46.69 |
| ATOM | 5926 | N | CYS | A | 762 | 72.765 | 40.013 | 47.599 | 1.00 | 36.07 |
| ATOM | 5927 | CA | CYS | A | 762 | 71.921 | 38.898 | 47.934 | 1.00 | 37.51 |
| ATOM | 5928 | C | CYS | A | 762 | 72.395 | 37.625 | 47.229 | 1.00 | 37.30 |
| ATOM | 5929 | O | CYS | A | 762 | 72.158 | 36.518 | 47.728 | 1.00 | 37.19 |
| ATOM | 5930 | CB | CYS | A | 762 | 70.457 | 39.208 | 47.609 | 1.00 | 37.88 |
| ATOM | 5931 | SG | CYS | A | 762 | 69.317 | 37.793 | 47.558 | 1.00 | 43.96 |
| ATOM | 5932 | N | PHE | A | 763 | 73.053 | 37.773 | 46.076 | 1.00 | 37.34 |
| ATOM | 5933 | CA | PHE | A | 763 | 73.506 | 36.622 | 45.313 | 1.00 | 37.27 |
| ATOM | 5934 | C | PHE | A | 763 | 74.982 | 36.320 | 45.533 | 1.00 | 38.08 |
| ATOM | 5935 | O | PHE | A | 763 | 75.516 | 35.436 | 44.902 | 1.00 | 37.30 |
| ATOM | 5936 | CB | PHE | A | 763 | 73.242 | 36.832 | 43.818 | 1.00 | 37.64 |
| ATOM | 5937 | CG | PHE | A | 763 | 71.803 | 36.621 | 43.415 | 1.00 | 37.37 |
| ATOM | 5938 | CD1 | PHE | A | 763 | 70.904 | 36.029 | 44.274 | 1.00 | 34.64 |
| ATOM | 5939 | CD2 | PHE | A | 763 | 71.349 | 37.052 | 42.189 | 1.00 | 36.85 |
| ATOM | 5940 | CE1 | PHE | A | 763 | 69.611 | 35.850 | 43.907 | 1.00 | 33.81 |
| ATOM | 5941 | CE2 | PHE | A | 763 | 70.032 | 36.872 | 41.828 | 1.00 | 35.30 |
| ATOM | 5942 | CZ | PHE | A | 763 | 69.176 | 36.272 | 42.691 | 1.00 | 33.56 |
| ATOM | 5943 | N | SER | A | 764 | 75.606 | 37.034 | 46.467 | 1.00 | 39.95 |
| ATOM | 5944 | CA | SER | A | 764 | 77.031 | 36.880 | 46.818 | 1.00 | 41.43 |
| ATOM | 5945 | C | SER | A | 764 | 77.910 | 37.043 | 45.578 | 1.00 | 42.27 |
| ATOM | 5946 | O | SER | A | 764 | 78.843 | 36.274 | 45.374 | 1.00 | 42.04 |
| ATOM | 5947 | CB | SER | A | 764 | 77.315 | 35.531 | 47.502 | 1.00 | 41.77 |
| ATOM | 5948 | OG | SER | A | 764 | 76.407 | 35.254 | 48.579 | 1.00 | 41.18 |
| ATOM | 5949 | N | LEU | A | 765 | 77.587 | 38.058 | 44.772 | 1.00 | 42.51 |
| ATOM | 5950 | CA | LEU | A | 765 | 78.294 | 38.354 | 43.535 | 1.00 | 43.43 |
| ATOM | 5951 | C | LEU | A | 765 | 79.064 | 39.660 | 43.666 | 1.00 | 44.05 |
| ATOM | 5952 | O | LEU | A | 765 | 78.499 | 40.660 | 44.070 | 1.00 | 43.63 |
| ATOM | 5953 | CB | LEU | A | 765 | 77.307 | 38.511 | 42.357 | 1.00 | 42.63 |
| ATOM | 5954 | CG | LEU | A | 765 | 76.470 | 37.312 | 41.885 | 1.00 | 43.40 |
| ATOM | 5955 | CD1 | LEU | A | 765 | 75.445 | 37.749 | 40.818 | 1.00 | 42.42 |
| ATOM | 5956 | CD2 | LEU | A | 765 | 77.358 | 36.214 | 41.312 | 1.00 | 44.39 |
| ATOM | 5957 | N | PRO | A | 766 | 80.352 | 39.649 | 43.334 | 1.00 | 45.52 |
| ATOM | 5958 | CA | PRO | A | 766 | 81.149 | 40.878 | 43.291 | 1.00 | 46.31 |
| ATOM | 5959 | C | PRO | A | 766 | 80.972 | 41.528 | 41.939 | 1.00 | 47.02 |
| ATOM | 5960 | O | PRO | A | 766 | 80.814 | 40.744 | 40.984 | 1.00 | 48.05 |
| ATOM | 5961 | CB | PRO | A | 766 | 82.582 | 40.368 | 43.425 | 1.00 | 46.75 |
| ATOM | 5962 | CG | PRO | A | 766 | 82.441 | 38.840 | 43.696 | 1.00 | 46.59 |
| ATOM | 5963 | CD | PRO | A | 766 | 81.168 | 38.462 | 43.016 | 1.00 | 45.98 |
| TER | 5964 | | PRO | A | 766 | | | | | |
| HETATM | 5965 | C1 | NAG | A | 793 | 52.247 | 84.441 | 26.665 | 1.00 | 56.57 |
| HETATM | 5966 | C2 | NAG | A | 793 | 51.667 | 85.774 | 26.181 | 1.00 | 59.26 |
| HETATM | 5967 | N2 | NAG | A | 793 | 50.405 | 85.614 | 25.454 | 1.00 | 60.75 |
| HETATM | 5968 | C7 | NAG | A | 793 | 50.230 | 84.786 | 24.417 | 1.00 | 63.64 |
| HETATM | 5969 | O7 | NAG | A | 793 | 49.104 | 84.445 | 24.028 | 1.00 | 64.39 |
| HETATM | 5970 | C8 | NAG | A | 793 | 51.434 | 84.255 | 23.678 | 1.00 | 63.71 |
| HETATM | 5971 | C3 | NAG | A | 793 | 52.732 | 86.604 | 25.440 | 1.00 | 59.62 |
| HETATM | 5972 | O3 | NAG | A | 793 | 52.304 | 87.904 | 25.060 | 1.00 | 59.46 |
| HETATM | 5973 | C4 | NAG | A | 793 | 53.931 | 86.799 | 26.333 | 1.00 | 59.23 |
| HETATM | 5974 | O4 | NAG | A | 793 | 54.958 | 87.354 | 25.538 | 1.00 | 58.87 |
| HETATM | 5975 | C5 | NAG | A | 793 | 54.379 | 85.491 | 26.977 | 1.00 | 58.73 |
| HETATM | 5976 | C6 | NAG | A | 793 | 55.422 | 85.799 | 28.061 | 1.00 | 58.77 |
| HETATM | 5977 | O6 | NAG | A | 793 | 54.806 | 86.202 | 29.259 | 1.00 | 58.36 |
| HETATM | 5978 | O5 | NAG | A | 793 | 53.306 | 84.773 | 27.544 | 1.00 | 56.72 |
| HETATM | 5979 | C1 | NAG | A | 794 | 57.357 | 62.419 | −5.828 | 1.00 | 28.91 |
| HETATM | 5980 | C2 | NAG | A | 794 | 57.044 | 63.800 | −5.253 | 1.00 | 30.20 |
| HETATM | 5981 | N2 | NAG | A | 794 | 56.632 | 63.635 | −3.866 | 1.00 | 29.11 |
| HETATM | 5982 | C7 | NAG | A | 794 | 57.358 | 63.964 | −2.815 | 1.00 | 29.67 |
| HETATM | 5983 | O7 | NAG | A | 794 | 58.514 | 64.379 | −2.847 | 1.00 | 28.47 |
| HETATM | 5984 | C8 | NAG | A | 794 | 56.666 | 63.783 | −1.481 | 1.00 | 30.86 |
| HETATM | 5985 | C3 | NAG | A | 794 | 55.889 | 64.431 | −6.033 | 1.00 | 31.06 |
| HETATM | 5986 | O3 | NAG | A | 794 | 55.644 | 65.736 | −5.613 | 1.00 | 32.22 |
| HETATM | 5987 | C4 | NAG | A | 794 | 56.322 | 64.529 | −7.468 | 1.00 | 32.03 |
| HETATM | 5988 | O4 | NAG | A | 794 | 55.313 | 65.150 | −8.198 | 1.00 | 30.75 |
| HETATM | 5989 | C5 | NAG | A | 794 | 56.558 | 63.108 | −7.965 | 1.00 | 32.35 |
| HETATM | 5990 | C6 | NAG | A | 794 | 56.903 | 63.109 | −9.455 | 1.00 | 32.88 |
| HETATM | 5991 | O6 | NAG | A | 794 | 57.858 | 64.097 | −9.728 | 1.00 | 30.65 |
| HETATM | 5992 | O5 | NAG | A | 794 | 57.632 | 62.574 | −7.216 | 1.00 | 31.57 |
| HETATM | 5993 | C1 | NAG | A | 795 | 26.557 | 83.475 | 27.320 | 1.00 | 69.38 |
| HETATM | 5994 | C2 | NAG | A | 795 | 26.517 | 84.675 | 28.278 | 1.00 | 70.37 |
| HETATM | 5995 | N2 | NAG | A | 795 | 27.031 | 85.876 | 27.627 | 1.00 | 71.29 |
| HETATM | 5996 | C7 | NAG | A | 795 | 26.337 | 86.484 | 26.653 | 1.00 | 72.14 |
| HETATM | 5997 | O7 | NAG | A | 795 | 25.108 | 86.415 | 26.530 | 1.00 | 71.13 |
| HETATM | 5998 | C8 | NAG | A | 795 | 27.135 | 87.272 | 25.659 | 1.00 | 72.66 |
| HETATM | 5999 | C3 | NAG | A | 795 | 27.147 | 84.328 | 29.631 | 1.00 | 68.73 |
| HETATM | 6000 | O3 | NAG | A | 795 | 27.036 | 85.420 | 30.524 | 1.00 | 67.24 |
| HETATM | 6001 | C4 | NAG | A | 795 | 26.366 | 83.126 | 30.165 | 1.00 | 68.79 |
| HETATM | 6002 | O4 | NAG | A | 795 | 26.805 | 82.703 | 31.436 | 1.00 | 65.52 |
| HETATM | 6003 | C5 | NAG | A | 795 | 26.453 | 81.990 | 29.151 | 1.00 | 70.52 |
| HETATM | 6004 | C6 | NAG | A | 795 | 25.734 | 80.729 | 29.625 | 1.00 | 71.77 |
| HETATM | 6005 | O6 | NAG | A | 795 | 25.527 | 79.863 | 28.524 | 1.00 | 71.65 |
| HETATM | 6006 | O5 | NAG | A | 795 | 25.881 | 82.386 | 27.919 | 1.00 | 70.37 |
| HETATM | 6007 | C1 | NAG | A | 796 | 28.778 | 69.824 | 39.914 | 1.00 | 33.92 |
| HETATM | 6008 | C2 | NAG | A | 796 | 27.615 | 70.692 | 39.410 | 1.00 | 35.69 |
| HETATM | 6009 | N2 | NAG | A | 796 | 28.001 | 71.731 | 38.471 | 1.00 | 33.86 |
| HETATM | 6010 | C7 | NAG | A | 796 | 27.907 | 71.604 | 37.160 | 1.00 | 34.06 |
| HETATM | 6011 | O7 | NAG | A | 796 | 27.527 | 70.594 | 36.590 | 1.00 | 36.08 |
| HETATM | 6012 | C8 | NAG | A | 796 | 28.341 | 72.778 | 36.352 | 1.00 | 36.76 |
| HETATM | 6013 | C3 | NAG | A | 796 | 27.015 | 71.446 | 40.578 | 1.00 | 38.11 |
| HETATM | 6014 | O3 | NAG | A | 796 | 25.987 | 72.255 | 40.072 | 1.00 | 38.60 |
| HETATM | 6015 | C4 | NAG | A | 796 | 26.563 | 70.490 | 41.666 | 1.00 | 40.14 |
| HETATM | 6016 | O4 | NAG | A | 796 | 26.063 | 71.140 | 42.848 | 1.00 | 44.49 |
| HETATM | 6017 | C5 | NAG | A | 796 | 27.784 | 69.659 | 42.014 | 1.00 | 40.15 |
| HETATM | 6018 | C6 | NAG | A | 796 | 27.444 | 68.688 | 43.129 | 1.00 | 40.02 |
| HETATM | 6019 | O6 | NAG | A | 796 | 26.267 | 68.081 | 42.668 | 1.00 | 44.11 |
| HETATM | 6020 | O5 | NAG | A | 796 | 28.232 | 68.954 | 40.876 | 1.00 | 33.47 |
| ATOM | 6021 | N | SER | B | 39 | 83.809 | 35.290 | 81.108 | 1.00 | 44.51 |
| ATOM | 6022 | CA | SER | B | 39 | 82.610 | 34.403 | 81.141 | 1.00 | 44.41 |
| ATOM | 6023 | C | SER | B | 39 | 81.248 | 35.137 | 81.269 | 1.00 | 44.06 |
| ATOM | 6024 | O | SER | B | 39 | 80.264 | 34.696 | 80.681 | 1.00 | 44.10 |
| ATOM | 6025 | CB | SER | B | 39 | 82.751 | 33.364 | 82.277 | 1.00 | 44.82 |
| ATOM | 6026 | OG | SER | B | 39 | 81.500 | 32.762 | 82.630 | 1.00 | 44.84 |
| ATOM | 6027 | N | ARG | B | 40 | 81.151 | 36.191 | 82.082 | 1.00 | 43.21 |
| ATOM | 6028 | CA | ARG | B | 40 | 79.877 | 36.926 | 82.188 | 1.00 | 42.08 |
| ATOM | 6029 | C | ARG | B | 40 | 79.254 | 37.325 | 80.843 | 1.00 | 40.45 |
| ATOM | 6030 | O | ARG | B | 40 | 79.926 | 37.440 | 79.822 | 1.00 | 39.04 |
| ATOM | 6031 | CB | ARG | B | 40 | 80.006 | 38.223 | 83.002 | 1.00 | 42.78 |
| ATOM | 6032 | CG | ARG | B | 40 | 80.757 | 38.172 | 84.357 | 1.00 | 43.26 |
| ATOM | 6033 | CD | ARG | B | 40 | 82.151 | 38.804 | 84.248 | 1.00 | 45.08 |
| ATOM | 6034 | NE | ARG | B | 40 | 82.289 | 40.180 | 84.761 | 1.00 | 46.19 |
| ATOM | 6035 | CZ | ARG | B | 40 | 83.237 | 41.042 | 84.357 | 1.00 | 47.79 |
| ATOM | 6036 | NH1 | ARG | B | 40 | 84.100 | 40.711 | 83.413 | 1.00 | 49.11 |
| ATOM | 6037 | NH2 | ARG | B | 40 | 83.318 | 42.256 | 84.868 | 1.00 | 49.15 |
| ATOM | 6038 | N | LYS | B | 41 | 77.947 | 37.556 | 80.852 | 1.00 | 39.26 |
| ATOM | 6039 | CA | LYS | B | 41 | 77.314 | 38.071 | 79.643 | 1.00 | 38.31 |
| ATOM | 6040 | C | LYS | B | 41 | 77.790 | 39.496 | 79.349 | 1.00 | 36.90 |
| ATOM | 6041 | O | LYS | B | 41 | 78.326 | 40.226 | 80.180 | 1.00 | 36.21 |
| ATOM | 6042 | CB | LYS | B | 41 | 75.796 | 38.000 | 79.712 | 1.00 | 38.22 |
| ATOM | 6043 | CG | LYS | B | 41 | 75.166 | 38.814 | 80.815 | 1.00 | 39.94 |
| ATOM | 6044 | CD | LYS | B | 41 | 73.659 | 38.723 | 80.787 | 1.00 | 42.54 |
| ATOM | 6045 | CE | LYS | B | 41 | 72.987 | 40.028 | 80.320 | 1.00 | 44.09 |
| ATOM | 6046 | NZ | LYS | B | 41 | 73.185 | 40.341 | 78.870 | 1.00 | 43.20 |
| ATOM | 6047 | N | THR | B | 42 | 77.593 | 39.860 | 78.118 | 1.00 | 35.87 |
| ATOM | 6048 | CA | THR | B | 42 | 77.981 | 41.136 | 77.584 | 1.00 | 35.53 |
| ATOM | 6049 | C | THR | B | 42 | 76.658 | 41.941 | 77.535 | 1.00 | 33.81 |
| ATOM | 6050 | O | THR | B | 42 | 75.604 | 41.329 | 77.564 | 1.00 | 34.18 |
| ATOM | 6051 | CB | THR | B | 42 | 78.542 | 40.805 | 76.215 | 1.00 | 36.04 |
| ATOM | 6052 | OG1 | THR | B | 42 | 79.892 | 41.260 | 76.064 | 1.00 | 38.02 |
| ATOM | 6053 | CG2 | THR | B | 42 | 77.750 | 41.422 | 75.134 | 1.00 | 36.36 |
| ATOM | 6054 | N | TYR | B | 43 | 76.712 | 43.277 | 77.534 | 1.00 | 31.50 |
| ATOM | 6055 | CA | TYR | B | 43 | 75.520 | 44.133 | 77.483 | 1.00 | 30.40 |
| ATOM | 6056 | C | TYR | B | 43 | 75.040 | 44.146 | 76.014 | 1.00 | 30.55 |
| ATOM | 6057 | O | TYR | B | 43 | 75.735 | 44.670 | 75.120 | 1.00 | 29.79 |
| ATOM | 6058 | CB | TYR | B | 43 | 75.864 | 45.557 | 77.976 | 1.00 | 29.97 |
| ATOM | 6059 | CG | TYR | B | 43 | 74.702 | 46.530 | 78.032 | 1.00 | 28.32 |
| ATOM | 6060 | CD1 | TYR | B | 43 | 73.805 | 46.508 | 79.066 | 1.00 | 26.99 |
| ATOM | 6061 | CD2 | TYR | B | 43 | 74.512 | 47.470 | 77.042 | 1.00 | 27.08 |
| ATOM | 6062 | CE1 | TYR | B | 43 | 72.715 | 47.391 | 79.109 | 1.00 | 27.36 |
| ATOM | 6063 | CE2 | TYR | B | 43 | 73.467 | 48.335 | 77.081 | 1.00 | 28.70 |
| ATOM | 6064 | CZ | TYR | B | 43 | 72.557 | 48.300 | 78.113 | 1.00 | 27.28 |
| ATOM | 6065 | OH | TYR | B | 43 | 71.501 | 49.199 | 78.147 | 1.00 | 28.22 |
| ATOM | 6066 | N | THR | B | 44 | 73.881 | 43.556 | 75.748 | 1.00 | 30.61 |
| ATOM | 6067 | CA | THR | B | 44 | 73.467 | 43.377 | 74.351 | 1.00 | 31.45 |
| ATOM | 6068 | C | THR | B | 44 | 72.530 | 44.459 | 73.804 | 1.00 | 31.97 |
| ATOM | 6069 | O | THR | B | 44 | 72.050 | 45.329 | 74.531 | 1.00 | 32.10 |
| ATOM | 6070 | CB | THR | B | 44 | 72.778 | 42.027 | 74.174 | 1.00 | 31.45 |
| ATOM | 6071 | OG1 | THR | B | 44 | 71.592 | 42.008 | 74.944 | 1.00 | 32.12 |
| ATOM | 6072 | CG2 | THR | B | 44 | 73.598 | 40.874 | 74.752 | 1.00 | 33.15 |
| ATOM | 6073 | N | LEU | B | 45 | 72.258 | 44.387 | 72.503 | 1.00 | 32.35 |
| ATOM | 6074 | CA | LEU | B | 45 | 71.322 | 45.317 | 71.886 | 1.00 | 32.17 |
| ATOM | 6075 | C | LEU | B | 45 | 69.951 | 45.080 | 72.518 | 1.00 | 32.45 |
| ATOM | 6076 | O | LEU | B | 45 | 69.219 | 46.016 | 72.820 | 1.00 | 34.45 |
| ATOM | 6077 | CB | LEU | B | 45 | 71.277 | 45.089 | 70.376 | 1.00 | 31.73 |
| ATOM | 6078 | CG | LEU | B | 45 | 70.268 | 45.920 | 69.570 | 1.00 | 31.61 |
| ATOM | 6079 | CD1 | LEU | B | 45 | 70.556 | 47.392 | 69.759 | 1.00 | 27.17 |
| ATOM | 6080 | CD2 | LEU | B | 45 | 70.220 | 45.528 | 68.036 | 1.00 | 31.95 |
| ATOM | 6081 | N | THR | B | 46 | 69.577 | 43.835 | 72.715 | 1.00 | 32.08 |
| ATOM | 6082 | CA | THR | B | 46 | 68.289 | 43.560 | 73.345 | 1.00 | 32.44 |
| ATOM | 6083 | C | THR | B | 46 | 68.264 | 44.073 | 74.798 | 1.00 | 31.32 |
| ATOM | 6084 | O | THR | B | 46 | 67.229 | 44.472 | 75.275 | 1.00 | 30.32 |
| ATOM | 6085 | CB | THR | B | 46 | 67.985 | 42.039 | 73.325 | 1.00 | 32.87 |
| ATOM | 6086 | OG1 | THR | B | 46 | 67.778 | 41.617 | 71.991 | 1.00 | 33.53 |
| ATOM | 6087 | CG2 | THR | B | 46 | 66.664 | 41.732 | 73.905 | 1.00 | 33.39 |
| ATOM | 6088 | N | ASP | B | 47 | 69.396 | 44.063 | 75.504 | 1.00 | 30.43 |
| ATOM | 6089 | CA | ASP | B | 47 | 69.383 | 44.642 | 76.843 | 1.00 | 30.27 |
| ATOM | 6090 | C | ASP | B | 47 | 69.012 | 46.124 | 76.753 | 1.00 | 29.58 |
| ATOM | 6091 | O | ASP | B | 47 | 68.184 | 46.602 | 77.495 | 1.00 | 28.74 |
| ATOM | 6092 | CB | ASP | B | 47 | 70.711 | 44.462 | 77.579 | 1.00 | 30.15 |
| ATOM | 6093 | CG | ASP | B | 47 | 70.990 | 43.013 | 77.958 | 1.00 | 29.26 |
| ATOM | 6094 | OD1 | ASP | B | 47 | 70.064 | 42.291 | 78.382 | 1.00 | 28.86 |
| ATOM | 6095 | OD2 | ASP | B | 47 | 72.127 | 42.522 | 77.875 | 1.00 | 29.48 |
| ATOM | 6096 | N | TYR | B | 48 | 69.570 | 46.824 | 75.786 | 1.00 | 29.34 |
| ATOM | 6097 | CA | TYR | B | 48 | 69.287 | 48.234 | 75.649 | 1.00 | 29.95 |
| ATOM | 6098 | C | TYR | B | 48 | 67.869 | 48.475 | 75.180 | 1.00 | 30.53 |
| ATOM | 6099 | O | TYR | B | 48 | 67.152 | 49.340 | 75.738 | 1.00 | 29.81 |
| ATOM | 6100 | CB | TYR | B | 48 | 70.275 | 48.878 | 74.675 | 1.00 | 30.20 |
| ATOM | 6101 | CG | TYR | B | 48 | 69.859 | 50.254 | 74.224 | 1.00 | 29.34 |
| ATOM | 6102 | CD1 | TYR | B | 48 | 69.649 | 51.273 | 75.144 | 1.00 | 28.33 |
| ATOM | 6103 | CD2 | TYR | B | 48 | 69.650 | 50.521 | 72.891 | 1.00 | 29.07 |
| ATOM | 6104 | CE1 | TYR | B | 48 | 69.270 | 52.514 | 74.737 | 1.00 | 27.28 |
| ATOM | 6105 | CE2 | TYR | B | 48 | 69.263 | 51.773 | 72.458 | 1.00 | 27.71 |
| ATOM | 6106 | CZ | TYR | B | 48 | 69.056 | 52.741 | 73.376 | 1.00 | 28.43 |
| ATOM | 6107 | OH | TYR | B | 48 | 68.681 | 53.952 | 72.932 | 1.00 | 31.85 |
| ATOM | 6108 | N | LEU | B | 49 | 67.438 | 47.687 | 74.195 | 1.00 | 30.94 |
| ATOM | 6109 | CA | LEU | B | 49 | 66.091 | 47.858 | 73.649 | 1.00 | 32.45 |
| ATOM | 6110 | C | LEU | B | 49 | 64.983 | 47.561 | 74.640 | 1.00 | 33.72 |
| ATOM | 6111 | O | LEU | B | 49 | 64.011 | 48.295 | 74.713 | 1.00 | 33.40 |
| ATOM | 6112 | CB | LEU | B | 49 | 65.920 | 46.998 | 72.387 | 1.00 | 32.26 |
| ATOM | 6113 | CG | LEU | B | 49 | 66.194 | 47.788 | 71.098 | 1.00 | 31.99 |
| ATOM | 6114 | CD1 | LEU | B | 49 | 67.040 | 48.975 | 71.308 | 1.00 | 29.89 |
| ATOM | 6115 | CD2 | LEU | B | 49 | 66.713 | 46.908 | 69.977 | 1.00 | 33.89 |
| ATOM | 6116 | N | LYS | B | 50 | 65.121 | 46.481 | 75.400 | 1.00 | 35.26 |
| ATOM | 6117 | CA | LYS | B | 50 | 64.090 | 46.107 | 76.374 | 1.00 | 37.22 |
| ATOM | 6118 | C | LYS | B | 50 | 64.293 | 46.640 | 77.806 | 1.00 | 37.29 |
| ATOM | 6119 | O | LYS | B | 50 | 63.612 | 46.202 | 78.711 | 1.00 | 37.02 |
| ATOM | 6120 | CB | LYS | B | 50 | 64.007 | 44.583 | 76.466 | 1.00 | 38.22 |
| ATOM | 6121 | CG | LYS | B | 50 | 63.593 | 43.860 | 75.230 | 1.00 | 40.87 |
| ATOM | 6122 | CD | LYS | B | 50 | 64.223 | 42.456 | 75.249 | 1.00 | 47.00 |
| ATOM | 6123 | CE | LYS | B | 50 | 63.689 | 41.520 | 76.384 | 1.00 | 49.69 |
| ATOM | 6124 | NZ | LYS | B | 50 | 64.640 | 40.396 | 76.655 | 1.00 | 49.70 |
| ATOM | 6125 | N | ASN | B | 51 | 65.261 | 47.520 | 78.031 | 1.00 | 38.20 |
| ATOM | 6126 | CA | ASN | B | 51 | 65.390 | 48.144 | 79.339 | 1.00 | 39.09 |
| ATOM | 6127 | C | ASN | B | 51 | 65.606 | 47.121 | 80.454 | 1.00 | 39.31 |
| ATOM | 6128 | O | ASN | B | 51 | 65.004 | 47.216 | 81.504 | 1.00 | 39.91 |
| ATOM | 6129 | CB | ASN | B | 51 | 64.085 | 48.890 | 79.618 | 1.00 | 39.37 |
| ATOM | 6130 | CG | ASN | B | 51 | 64.298 | 50.254 | 80.233 | 1.00 | 42.26 |
| ATOM | 6131 | OD1 | ASN | B | 51 | 63.738 | 50.571 | 81.293 | 1.00 | 46.59 |
| ATOM | 6132 | ND2 | ASN | B | 51 | 65.079 | 51.081 | 79.569 | 1.00 | 42.03 |
| ATOM | 6133 | N | THR | B | 52 | 66.453 | 46.134 | 80.227 | 1.00 | 39.39 |
| ATOM | 6134 | CA | THR | B | 52 | 66.643 | 45.080 | 81.204 | 1.00 | 39.63 |
| ATOM | 6135 | C | THR | B | 52 | 67.329 | 45.541 | 82.496 | 1.00 | 39.19 |
| ATOM | 6136 | O | THR | B | 52 | 67.029 | 45.023 | 83.560 | 1.00 | 38.43 |
| ATOM | 6137 | CB | THR | B | 52 | 67.446 | 43.970 | 80.573 | 1.00 | 39.59 |
| ATOM | 6138 | OG1 | THR | B | 52 | 66.824 | 43.606 | 79.348 | 1.00 | 40.44 |
| ATOM | 6139 | CG2 | THR | B | 52 | 67.349 | 42.695 | 81.412 | 1.00 | 40.57 |
| ATOM | 6140 | N | TYR | B | 53 | 68.240 | 46.497 | 82.356 | 1.00 | 38.59 |
| ATOM | 6141 | CA | TYR | B | 53 | 68.989 | 47.077 | 83.443 | 1.00 | 38.99 |
| ATOM | 6142 | C | TYR | B | 53 | 68.498 | 48.514 | 83.663 | 1.00 | 39.15 |
| ATOM | 6143 | O | TYR | B | 53 | 68.932 | 49.451 | 82.998 | 1.00 | 39.02 |
| ATOM | 6144 | CB | TYR | B | 53 | 70.484 | 46.999 | 83.109 | 1.00 | 38.55 |
| ATOM | 6145 | CG | TYR | B | 53 | 70.948 | 45.564 | 82.960 | 1.00 | 38.41 |
| ATOM | 6146 | CD1 | TYR | B | 53 | 70.925 | 44.687 | 84.034 | 1.00 | 39.45 |
| ATOM | 6147 | CD2 | TYR | B | 53 | 71.368 | 45.067 | 81.733 | 1.00 | 40.02 |
| ATOM | 6148 | CE1 | TYR | B | 53 | 71.337 | 43.341 | 83.885 | 1.00 | 40.17 |
| ATOM | 6149 | CE2 | TYR | B | 53 | 71.769 | 43.737 | 81.580 | 1.00 | 39.74 |
| ATOM | 6150 | CZ | TYR | B | 53 | 71.749 | 42.889 | 82.650 | 1.00 | 39.48 |
| ATOM | 6151 | OH | TYR | B | 53 | 72.159 | 41.588 | 82.477 | 1.00 | 41.16 |
| ATOM | 6152 | N | ARG | B | 54 | 67.580 | 48.668 | 84.606 | 1.00 | 39.82 |
| ATOM | 6153 | CA | ARG | B | 54 | 66.880 | 49.929 | 84.811 | 1.00 | 41.09 |
| ATOM | 6154 | C | ARG | B | 54 | 67.437 | 50.820 | 85.918 | 1.00 | 39.95 |
| ATOM | 6155 | O | ARG | B | 54 | 67.650 | 50.345 | 87.042 | 1.00 | 40.34 |
| ATOM | 6156 | CB | ARG | B | 54 | 65.419 | 49.637 | 85.167 | 1.00 | 42.08 |
| ATOM | 6157 | CG | ARG | B | 54 | 64.600 | 49.054 | 84.064 | 1.00 | 48.62 |
| ATOM | 6158 | CD | ARG | B | 54 | 63.077 | 48.880 | 84.417 | 1.00 | 54.89 |
| ATOM | 6159 | NE | ARG | B | 54 | 62.411 | 47.906 | 83.531 | 1.00 | 59.33 |
| ATOM | 6160 | CZ | ARG | B | 54 | 61.659 | 48.219 | 82.463 | 1.00 | 63.36 |
| ATOM | 6161 | NH1 | ARG | B | 54 | 61.460 | 49.488 | 82.109 | 1.00 | 64.50 |
| ATOM | 6162 | NH2 | ARG | B | 54 | 61.103 | 47.254 | 81.736 | 1.00 | 64.31 |
| ATOM | 6163 | N | LEU | B | 55 | 67.624 | 52.099 | 85.602 | 1.00 | 38.60 |
| ATOM | 6164 | CA | LEU | B | 55 | 68.019 | 53.106 | 86.583 | 1.00 | 38.98 |
| ATOM | 6165 | C | LEU | B | 55 | 66.848 | 53.477 | 87.458 | 1.00 | 38.29 |
| ATOM | 6166 | O | LEU | B | 55 | 65.761 | 53.777 | 86.954 | 1.00 | 37.51 |
| ATOM | 6167 | CB | LEU | B | 55 | 68.541 | 54.365 | 85.898 | 1.00 | 38.96 |
| ATOM | 6168 | CG | LEU | B | 55 | 69.895 | 54.108 | 85.263 | 1.00 | 41.05 |
| ATOM | 6169 | CD1 | LEU | B | 55 | 70.193 | 55.111 | 84.136 | 1.00 | 43.07 |
| ATOM | 6170 | CD2 | LEU | B | 55 | 70.922 | 54.157 | 86.320 | 1.00 | 41.53 |
| ATOM | 6171 | N | LYS | B | 56 | 67.047 | 53.395 | 88.774 | 1.00 | 37.79 |
| ATOM | 6172 | CA | LYS | B | 56 | 65.993 | 53.746 | 89.732 | 1.00 | 37.45 |
| ATOM | 6173 | C | LYS | B | 56 | 66.122 | 55.239 | 90.005 | 1.00 | 36.19 |
| ATOM | 6174 | O | LYS | B | 56 | 67.226 | 55.745 | 90.142 | 1.00 | 36.81 |
| ATOM | 6175 | CB | LYS | B | 56 | 66.093 | 52.962 | 91.048 | 1.00 | 37.81 |
| ATOM | 6176 | CG | LYS | B | 56 | 65.489 | 51.564 | 91.056 | 1.00 | 40.35 |
| ATOM | 6177 | CD | LYS | B | 56 | 65.304 | 51.064 | 92.507 | 1.00 | 44.36 |
| ATOM | 6178 | CE | LYS | B | 56 | 65.245 | 49.527 | 92.650 | 1.00 | 46.29 |
| ATOM | 6179 | NZ | LYS | B | 56 | 65.354 | 49.015 | 94.089 | 1.00 | 43.46 |
| ATOM | 6180 | N | LEU | B | 57 | 64.976 | 55.903 | 90.107 | 1.00 | 34.92 |
| ATOM | 6181 | CA | LEU | B | 57 | 64.854 | 57.353 | 90.238 | 1.00 | 34.55 |
| ATOM | 6182 | C | LEU | B | 57 | 64.324 | 57.683 | 91.612 | 1.00 | 32.13 |
| ATOM | 6183 | O | LEU | B | 57 | 63.927 | 56.808 | 92.336 | 1.00 | 32.19 |
| ATOM | 6184 | CB | LEU | B | 57 | 63.815 | 57.886 | 89.209 | 1.00 | 34.23 |
| ATOM | 6185 | CG | LEU | B | 57 | 63.956 | 57.325 | 87.791 | 1.00 | 38.69 |
| ATOM | 6186 | CD1 | LEU | B | 57 | 62.694 | 57.519 | 86.874 | 1.00 | 40.17 |
| ATOM | 6187 | CD2 | LEU | B | 57 | 65.175 | 57.973 | 87.144 | 1.00 | 38.67 |
| ATOM | 6188 | N | TYR | B | 58 | 64.366 | 58.951 | 91.968 | 1.00 | 30.33 |
| ATOM | 6189 | CA | TYR | B | 58 | 63.645 | 59.437 | 93.133 | 1.00 | 28.99 |
| ATOM | 6190 | C | TYR | B | 58 | 63.147 | 60.832 | 92.827 | 1.00 | 28.96 |
| ATOM | 6191 | O | TYR | B | 58 | 63.755 | 61.816 | 93.195 | 1.00 | 28.57 |
| ATOM | 6192 | CB | TYR | B | 58 | 64.489 | 59.430 | 94.405 | 1.00 | 28.55 |
| ATOM | 6193 | CG | TYR | B | 58 | 63.678 | 59.376 | 95.687 | 1.00 | 26.14 |
| ATOM | 6194 | CD1 | TYR | B | 58 | 63.157 | 60.541 | 96.221 | 1.00 | 26.40 |
| ATOM | 6195 | CD2 | TYR | B | 58 | 63.436 | 58.166 | 96.362 | 1.00 | 25.89 |
| ATOM | 6196 | CE1 | TYR | B | 58 | 62.428 | 60.550 | 97.356 | 1.00 | 25.05 |
| ATOM | 6197 | CE2 | TYR | B | 58 | 62.668 | 58.141 | 97.571 | 1.00 | 24.22 |
| ATOM | 6198 | CZ | TYR | B | 58 | 62.169 | 59.359 | 98.037 | 1.00 | 27.02 |
| ATOM | 6199 | OH | TYR | B | 58 | 61.443 | 59.503 | 99.176 | 1.00 | 27.44 |
| ATOM | 6200 | N | SER | B | 59 | 62.014 | 60.891 | 92.154 | 1.00 | 28.77 |
| ATOM | 6201 | CA | SER | B | 59 | 61.351 | 62.127 | 91.819 | 1.00 | 28.72 |
| ATOM | 6202 | C | SER | B | 59 | 60.397 | 62.561 | 92.896 | 1.00 | 28.49 |
| ATOM | 6203 | O | SER | B | 59 | 59.401 | 61.897 | 93.153 | 1.00 | 27.25 |
| ATOM | 6204 | CB | SER | B | 59 | 60.541 | 61.917 | 90.537 | 1.00 | 29.06 |
| ATOM | 6205 | OG | SER | B | 59 | 61.360 | 61.214 | 89.608 | 1.00 | 31.54 |
| ATOM | 6206 | N | LEU | B | 60 | 60.662 | 63.723 | 93.479 | 1.00 | 28.81 |
| ATOM | 6207 | CA | LEU | B | 60 | 59.803 | 64.224 | 94.518 | 1.00 | 29.07 |
| ATOM | 6208 | C | LEU | B | 60 | 59.311 | 65.609 | 94.189 | 1.00 | 30.02 |
| ATOM | 6209 | O | LEU | B | 60 | 59.855 | 66.219 | 93.299 | 1.00 | 29.55 |
| ATOM | 6210 | CB | LEU | B | 60 | 60.532 | 64.214 | 95.864 | 1.00 | 28.50 |
| ATOM | 6211 | CG | LEU | B | 60 | 61.605 | 65.189 | 96.362 | 1.00 | 29.47 |
| ATOM | 6212 | CD1 | LEU | B | 60 | 62.895 | 64.477 | 96.481 | 1.00 | 32.38 |
| ATOM | 6213 | CD2 | LEU | B | 60 | 61.809 | 66.511 | 95.678 | 1.00 | 28.57 |
| ATOM | 6214 | N | ARG | B | 61 | 58.277 | 66.087 | 94.889 | 1.00 | 30.95 |
| ATOM | 6215 | CA | ARG | B | 61 | 57.791 | 67.457 | 94.701 | 1.00 | 32.76 |
| ATOM | 6216 | C | ARG | B | 61 | 57.674 | 68.106 | 96.066 | 1.00 | 31.69 |
| ATOM | 6217 | O | ARG | B | 61 | 56.884 | 67.687 | 96.880 | 1.00 | 32.62 |
| ATOM | 6218 | CB | ARG | B | 61 | 56.437 | 67.550 | 93.945 | 1.00 | 33.00 |
| ATOM | 6219 | CG | ARG | B | 61 | 56.301 | 66.653 | 92.722 | 1.00 | 37.71 |
| ATOM | 6220 | CD | ARG | B | 61 | 54.916 | 66.765 | 91.970 | 1.00 | 43.84 |
| ATOM | 6221 | NE | ARG | B | 61 | 54.765 | 65.788 | 90.874 | 1.00 | 46.60 |
| ATOM | 6222 | CZ | ARG | B | 61 | 53.631 | 65.576 | 90.187 | 1.00 | 48.81 |
| ATOM | 6223 | NH1 | ARG | B | 61 | 52.527 | 66.245 | 90.482 | 1.00 | 50.54 |
| ATOM | 6224 | NH2 | ARG | B | 61 | 53.592 | 64.681 | 89.209 | 1.00 | 49.42 |
| ATOM | 6225 | N | TRP | B | 62 | 58.489 | 69.106 | 96.305 | 1.00 | 30.71 |
| ATOM | 6226 | CA | TRP | B | 62 | 58.431 | 69.858 | 97.528 | 1.00 | 31.67 |
| ATOM | 6227 | C | TRP | B | 62 | 57.093 | 70.552 | 97.627 | 1.00 | 31.76 |
| ATOM | 6228 | O | TRP | B | 62 | 56.615 | 71.148 | 96.684 | 1.00 | 30.27 |
| ATOM | 6229 | CB | TRP | B | 62 | 59.554 | 70.906 | 97.586 | 1.00 | 30.92 |
| ATOM | 6230 | CG | TRP | B | 62 | 60.884 | 70.294 | 97.811 | 1.00 | 31.46 |
| ATOM | 6231 | CD1 | TRP | B | 62 | 61.894 | 70.191 | 96.920 | 1.00 | 29.49 |
| ATOM | 6232 | CD2 | TRP | B | 62 | 61.346 | 69.658 | 99.022 | 1.00 | 31.13 |
| ATOM | 6233 | NE1 | TRP | B | 62 | 62.968 | 69.561 | 97.503 | 1.00 | 31.80 |
| ATOM | 6234 | CE2 | TRP | B | 62 | 62.655 | 69.210 | 98.784 | 1.00 | 29.69 |
| ATOM | 6235 | CE3 | TRP | B | 62 | 60.786 | 69.446 | 100.281 | 1.00 | 26.50 |
| ATOM | 6236 | CZ2 | TRP | B | 62 | 63.416 | 68.553 | 99.752 | 1.00 | 31.02 |
| ATOM | 6237 | CZ3 | TRP | B | 62 | 61.544 | 68.796 | 101.258 | 1.00 | 31.33 |
| ATOM | 6238 | CH2 | TRP | B | 62 | 62.843 | 68.360 | 100.991 | 1.00 | 31.10 |
| ATOM | 6239 | N | ILE | B | 63 | 56.513 | 70.510 | 98.803 | 1.00 | 32.74 |
| ATOM | 6240 | CA | ILE | B | 63 | 55.209 | 71.110 | 98.987 | 1.00 | 33.71 |
| ATOM | 6241 | C | ILE | B | 63 | 55.150 | 72.147 | 100.103 | 1.00 | 33.06 |
| ATOM | 6242 | O | ILE | B | 63 | 54.101 | 72.746 | 100.340 | 1.00 | 32.38 |
| ATOM | 6243 | CB | ILE | B | 63 | 54.258 | 69.939 | 99.191 | 1.00 | 34.64 |
| ATOM | 6244 | CG1 | ILE | B | 63 | 53.250 | 69.964 | 98.088 | 1.00 | 36.41 |
| ATOM | 6245 | CG2 | ILE | B | 63 | 53.749 | 69.781 | 100.673 | 1.00 | 35.85 |
| ATOM | 6246 | CD1 | ILE | B | 63 | 52.966 | 68.618 | 97.611 | 1.00 | 38.12 |
| ATOM | 6247 | N | SER | B | 64 | 56.282 | 72.344 | 100.773 | 1.00 | 31.88 |
| ATOM | 6248 | CA | SER | B | 64 | 56.416 | 73.345 | 101.799 | 1.00 | 32.02 |
| ATOM | 6249 | C | SER | B | 64 | 57.886 | 73.450 | 102.100 | 1.00 | 32.02 |
| ATOM | 6250 | O | SER | B | 64 | 58.678 | 73.007 | 101.290 | 1.00 | 31.20 |
| ATOM | 6251 | CB | SER | B | 64 | 55.628 | 72.939 | 103.047 | 1.00 | 32.59 |
| ATOM | 6252 | OG | SER | B | 64 | 56.135 | 71.745 | 103.603 | 1.00 | 30.59 |
| ATOM | 6253 | N | ASP | B | 65 | 58.261 | 74.008 | 103.249 | 1.00 | 33.10 |
| ATOM | 6254 | CA | ASP | B | 65 | 59.676 | 74.070 | 103.598 | 1.00 | 34.48 |
| ATOM | 6255 | C | ASP | B | 65 | 60.244 | 72.806 | 104.207 | 1.00 | 33.71 |
| ATOM | 6256 | O | ASP | B | 65 | 61.452 | 72.723 | 104.392 | 1.00 | 33.81 |
| ATOM | 6257 | CB | ASP | B | 65 | 59.967 | 75.243 | 104.542 | 1.00 | 35.30 |
| ATOM | 6258 | CG | ASP | B | 65 | 59.330 | 75.094 | 105.936 | 1.00 | 39.93 |
| ATOM | 6259 | OD1 | ASP | B | 65 | 58.317 | 74.376 | 106.106 | 1.00 | 41.80 |
| ATOM | 6260 | OD2 | ASP | B | 65 | 59.777 | 75.718 | 106.938 | 1.00 | 46.16 |
| ATOM | 6261 | N | HIS | B | 66 | 59.391 | 71.842 | 104.520 | 1.00 | 33.76 |
| ATOM | 6262 | CA | HIS | B | 66 | 59.865 | 70.641 | 105.209 | 1.00 | 34.50 |
| ATOM | 6263 | C | HIS | B | 66 | 59.224 | 69.281 | 104.805 | 1.00 | 33.72 |
| ATOM | 6264 | O | HIS | B | 66 | 59.443 | 68.274 | 105.458 | 1.00 | 33.16 |
| ATOM | 6265 | CB | HIS | B | 66 | 59.711 | 70.884 | 106.716 | 1.00 | 35.88 |
| ATOM | 6266 | CG | HIS | B | 66 | 58.295 | 70.884 | 107.202 | 1.00 | 38.04 |
| ATOM | 6267 | ND1 | HIS | B | 66 | 57.772 | 69.855 | 107.963 | 1.00 | 45.25 |
| ATOM | 6268 | CD2 | HIS | B | 66 | 57.312 | 71.810 | 107.098 | 1.00 | 43.06 |
| ATOM | 6269 | CE1 | HIS | B | 66 | 56.510 | 70.127 | 108.264 | 1.00 | 46.62 |
| ATOM | 6270 | NE2 | HIS | B | 66 | 56.205 | 71.309 | 107.751 | 1.00 | 46.13 |
| ATOM | 6271 | N | GLU | B | 67 | 58.439 | 69.283 | 103.730 | 1.00 | 32.69 |
| ATOM | 6272 | CA | GLU | B | 67 | 57.729 | 68.118 | 103.271 | 1.00 | 33.17 |
| ATOM | 6273 | C | GLU | B | 67 | 57.654 | 68.046 | 101.761 | 1.00 | 31.79 |
| ATOM | 6274 | O | GLU | B | 67 | 57.596 | 69.071 | 101.075 | 1.00 | 31.54 |
| ATOM | 6275 | CB | GLU | B | 67 | 56.293 | 68.079 | 103.791 | 1.00 | 33.48 |
| ATOM | 6276 | CG | GLU | B | 67 | 56.113 | 68.009 | 105.296 | 1.00 | 37.72 |
| ATOM | 6277 | CD | GLU | B | 67 | 54.656 | 68.234 | 105.672 | 1.00 | 42.22 |
| ATOM | 6278 | OE1 | GLU | B | 67 | 54.164 | 69.349 | 105.378 | 1.00 | 48.76 |
| ATOM | 6279 | OE2 | GLU | B | 67 | 53.997 | 67.321 | 106.228 | 1.00 | 42.33 |
| ATOM | 6280 | N | TYR | B | 68 | 57.631 | 66.824 | 101.256 | 1.00 | 30.97 |
| ATOM | 6281 | CA | TYR | B | 68 | 57.509 | 66.585 | 99.825 | 1.00 | 29.71 |
| ATOM | 6282 | C | TYR | B | 68 | 56.596 | 65.424 | 99.528 | 1.00 | 29.93 |
| ATOM | 6283 | O | TYR | B | 68 | 56.321 | 64.578 | 100.413 | 1.00 | 29.08 |
| ATOM | 6284 | CB | TYR | B | 68 | 58.873 | 66.327 | 99.185 | 1.00 | 29.52 |
| ATOM | 6285 | CG | TYR | B | 68 | 59.668 | 65.092 | 99.671 | 1.00 | 28.39 |
| ATOM | 6286 | CD1 | TYR | B | 68 | 59.365 | 63.817 | 99.220 | 1.00 | 22.82 |
| ATOM | 6287 | CD2 | TYR | B | 68 | 60.730 | 65.231 | 100.587 | 1.00 | 28.35 |
| ATOM | 6288 | CE1 | TYR | B | 68 | 60.087 | 62.708 | 99.624 | 1.00 | 23.16 |
| ATOM | 6289 | CE2 | TYR | B | 68 | 61.479 | 64.106 | 101.005 | 1.00 | 29.42 |
| ATOM | 6290 | CZ | TYR | B | 68 | 61.137 | 62.848 | 100.526 | 1.00 | 29.06 |
| ATOM | 6291 | OH | TYR | B | 68 | 61.857 | 61.741 | 100.912 | 1.00 | 26.61 |
| ATOM | 6292 | N | LEU | B | 69 | 56.127 | 65.381 | 98.281 | 1.00 | 29.92 |
| ATOM | 6293 | CA | LEU | B | 69 | 55.358 | 64.233 | 97.786 | 1.00 | 30.28 |
| ATOM | 6294 | C | LEU | B | 69 | 56.188 | 63.278 | 96.978 | 1.00 | 30.18 |
| ATOM | 6295 | O | LEU | B | 69 | 57.070 | 63.652 | 96.218 | 1.00 | 30.00 |
| ATOM | 6296 | CB | LEU | B | 69 | 54.194 | 64.664 | 96.895 | 1.00 | 30.22 |
| ATOM | 6297 | CG | LEU | B | 69 | 53.193 | 65.594 | 97.530 | 1.00 | 30.36 |
| ATOM | 6298 | CD1 | LEU | B | 69 | 52.388 | 66.250 | 96.405 | 1.00 | 34.17 |
| ATOM | 6299 | CD2 | LEU | B | 69 | 52.338 | 64.841 | 98.468 | 1.00 | 31.47 |
| ATOM | 6300 | N | TYR | B | 70 | 55.833 | 62.024 | 97.085 | 1.00 | 31.67 |
| ATOM | 6301 | CA | TYR | B | 70 | 56.553 | 60.971 | 96.424 | 1.00 | 33.09 |
| ATOM | 6302 | C | TYR | B | 70 | 55.561 | 59.866 | 96.272 | 1.00 | 34.91 |
| ATOM | 6303 | O | TYR | B | 70 | 54.762 | 59.608 | 97.179 | 1.00 | 35.21 |
| ATOM | 6304 | CB | TYR | B | 70 | 57.730 | 60.511 | 97.296 | 1.00 | 33.20 |
| ATOM | 6305 | CG | TYR | B | 70 | 58.487 | 59.371 | 96.681 | 1.00 | 34.09 |
| ATOM | 6306 | CD1 | TYR | B | 70 | 59.342 | 59.571 | 95.613 | 1.00 | 34.32 |
| ATOM | 6307 | CD2 | TYR | B | 70 | 58.333 | 58.088 | 97.159 | 1.00 | 36.48 |
| ATOM | 6308 | CE1 | TYR | B | 70 | 60.027 | 58.518 | 95.043 | 1.00 | 35.68 |
| ATOM | 6309 | CE2 | TYR | B | 70 | 59.017 | 57.035 | 96.607 | 1.00 | 38.59 |
| ATOM | 6310 | CZ | TYR | B | 70 | 59.859 | 57.251 | 95.554 | 1.00 | 38.10 |
| ATOM | 6311 | OH | TYR | B | 70 | 60.530 | 56.175 | 95.042 | 1.00 | 38.44 |
| ATOM | 6312 | N | LYS | B | 71 | 55.580 | 59.217 | 95.132 | 1.00 | 37.42 |
| ATOM | 6313 | CA | LYS | B | 71 | 54.665 | 58.132 | 94.922 | 1.00 | 39.82 |
| ATOM | 6314 | C | LYS | B | 71 | 55.395 | 56.788 | 95.087 | 1.00 | 41.07 |
| ATOM | 6315 | O | LYS | B | 71 | 56.472 | 56.572 | 94.523 | 1.00 | 40.84 |
| ATOM | 6316 | CB | LYS | B | 71 | 53.902 | 58.294 | 93.598 | 1.00 | 40.28 |
| ATOM | 6317 | CG | LYS | B | 71 | 54.600 | 57.907 | 92.345 | 1.00 | 43.25 |
| ATOM | 6318 | CD | LYS | B | 71 | 53.531 | 57.754 | 91.197 | 1.00 | 48.13 |
| ATOM | 6319 | CE | LYS | B | 71 | 53.486 | 58.966 | 90.240 | 1.00 | 48.89 |
| ATOM | 6320 | NZ | LYS | B | 71 | 53.741 | 60.292 | 90.932 | 1.00 | 47.42 |
| ATOM | 6321 | N | GLN | B | 72 | 54.823 | 55.926 | 95.926 | 1.00 | 42.40 |
| ATOM | 6322 | CA | GLN | B | 72 | 55.399 | 54.622 | 96.244 | 1.00 | 44.10 |
| ATOM | 6323 | C | GLN | B | 72 | 54.321 | 53.572 | 96.095 | 1.00 | 45.27 |
| ATOM | 6324 | O | GLN | B | 72 | 53.281 | 53.651 | 96.762 | 1.00 | 44.58 |
| ATOM | 6325 | CB | GLN | B | 72 | 55.910 | 54.632 | 97.691 | 1.00 | 44.50 |
| ATOM | 6326 | CG | GLN | B | 72 | 56.800 | 53.468 | 98.088 | 1.00 | 44.48 |
| ATOM | 6327 | CD | GLN | B | 72 | 57.329 | 53.630 | 99.503 | 1.00 | 44.96 |
| ATOM | 6328 | OE1 | GLN | B | 72 | 56.615 | 53.373 | 100.474 | 1.00 | 43.70 |
| ATOM | 6329 | NE2 | GLN | B | 72 | 58.576 | 54.051 | 99.621 | 1.00 | 45.53 |
| ATOM | 6330 | N | GLU | B | 73 | 54.569 | 52.601 | 95.211 | 1.00 | 47.20 |
| ATOM | 6331 | CA | GLU | B | 73 | 53.630 | 51.505 | 94.957 | 1.00 | 48.73 |
| ATOM | 6332 | C | GLU | B | 73 | 52.286 | 52.156 | 94.671 | 1.00 | 48.68 |
| ATOM | 6333 | O | GLU | B | 73 | 51.254 | 51.786 | 95.215 | 1.00 | 48.41 |
| ATOM | 6334 | CB | GLU | B | 73 | 53.574 | 50.546 | 96.158 | 1.00 | 49.31 |
| ATOM | 6335 | CG | GLU | B | 73 | 54.947 | 50.028 | 96.582 | 1.00 | 51.35 |
| ATOM | 6336 | CD | GLU | B | 73 | 54.871 | 48.833 | 97.530 | 1.00 | 54.65 |
| ATOM | 6337 | OE1 | GLU | B | 73 | 54.276 | 47.787 | 97.142 | 1.00 | 55.54 |
| ATOM | 6338 | OE2 | GLU | B | 73 | 55.408 | 48.943 | 98.663 | 1.00 | 54.55 |
| ATOM | 6339 | N | ASN | B | 74 | 52.366 | 53.079 | 93.718 | 1.00 | 49.04 |
| ATOM | 6340 | CA | ASN | B | 74 | 51.376 | 54.107 | 93.416 | 1.00 | 49.01 |
| ATOM | 6341 | C | ASN | B | 74 | 50.382 | 54.603 | 94.488 | 1.00 | 47.27 |
| ATOM | 6342 | O | ASN | B | 74 | 49.229 | 54.930 | 94.228 | 1.00 | 46.85 |
| ATOM | 6343 | CB | ASN | B | 74 | 50.854 | 54.005 | 91.978 | 1.00 | 50.33 |
| ATOM | 6344 | CG | ASN | B | 74 | 51.795 | 54.740 | 91.011 | 1.00 | 53.96 |
| ATOM | 6345 | OD1 | ASN | B | 74 | 53.026 | 54.631 | 91.147 | 1.00 | 59.25 |
| ATOM | 6346 | ND2 | ASN | B | 74 | 51.243 | 55.541 | 90.102 | 1.00 | 58.66 |
| ATOM | 6347 | N | ASN | B | 75 | 50.912 | 54.711 | 95.701 | 1.00 | 45.27 |
| ATOM | 6348 | CA | ASN | B | 75 | 50.285 | 55.498 | 96.736 | 1.00 | 43.89 |
| ATOM | 6349 | C | ASN | B | 75 | 50.971 | 56.866 | 96.625 | 1.00 | 42.53 |
| ATOM | 6350 | O | ASN | B | 75 | 52.152 | 56.948 | 96.357 | 1.00 | 42.77 |
| ATOM | 6351 | CB | ASN | B | 75 | 50.559 | 54.934 | 98.133 | 1.00 | 43.56 |
| ATOM | 6352 | CG | ASN | B | 75 | 49.860 | 53.600 | 98.387 | 1.00 | 42.32 |
| ATOM | 6353 | OD1 | ASN | B | 75 | 48.634 | 53.518 | 98.429 | 1.00 | 38.62 |
| ATOM | 6354 | ND2 | ASN | B | 75 | 50.651 | 52.560 | 98.593 | 1.00 | 43.37 |
| ATOM | 6355 | N | ILE | B | 76 | 50.231 | 57.940 | 96.807 | 1.00 | 40.69 |
| ATOM | 6356 | CA | ILE | B | 76 | 50.842 | 59.233 | 96.857 | 1.00 | 38.95 |
| ATOM | 6357 | C | ILE | B | 76 | 51.150 | 59.520 | 98.314 | 1.00 | 36.65 |
| ATOM | 6358 | O | ILE | B | 76 | 50.255 | 59.693 | 99.109 | 1.00 | 34.71 |
| ATOM | 6359 | CB | ILE | B | 76 | 49.910 | 60.272 | 96.247 | 1.00 | 39.60 |
| ATOM | 6360 | CG1 | ILE | B | 76 | 49.892 | 60.098 | 94.722 | 1.00 | 40.79 |
| ATOM | 6361 | CG2 | ILE | B | 76 | 50.395 | 61.665 | 96.574 | 1.00 | 40.25 |
| ATOM | 6362 | CD1 | ILE | B | 76 | 49.030 | 61.130 | 94.000 | 1.00 | 43.78 |
| ATOM | 6363 | N | LEU | B | 77 | 52.433 | 59.560 | 98.653 | 1.00 | 34.92 |
| ATOM | 6364 | CA | LEU | B | 77 | 52.846 | 59.775 | 100.024 | 1.00 | 33.64 |
| ATOM | 6365 | C | LEU | B | 77 | 53.402 | 61.163 | 100.234 | 1.00 | 33.19 |
| ATOM | 6366 | O | LEU | B | 77 | 53.918 | 61.786 | 99.277 | 1.00 | 32.58 |
| ATOM | 6367 | CB | LEU | B | 77 | 53.966 | 58.807 | 100.384 | 1.00 | 33.67 |
| ATOM | 6368 | CG | LEU | B | 77 | 53.902 | 57.317 | 100.033 | 1.00 | 34.31 |
| ATOM | 6369 | CD1 | LEU | B | 77 | 55.169 | 56.615 | 100.550 | 1.00 | 34.52 |
| ATOM | 6370 | CD2 | LEU | B | 77 | 52.668 | 56.671 | 100.612 | 1.00 | 35.27 |
| ATOM | 6371 | N | VAL | B | 78 | 53.250 | 61.669 | 101.463 | 1.00 | 32.23 |
| ATOM | 6372 | CA | VAL | B | 78 | 54.007 | 62.830 | 101.890 | 1.00 | 32.17 |
| ATOM | 6373 | C | VAL | B | 78 | 55.108 | 62.367 | 102.840 | 1.00 | 31.20 |
| ATOM | 6374 | O | VAL | B | 78 | 54.875 | 61.585 | 103.751 | 1.00 | 30.88 |
| ATOM | 6375 | CB | VAL | B | 78 | 53.184 | 63.855 | 102.689 | 1.00 | 33.20 |
| ATOM | 6376 | CG1 | VAL | B | 78 | 54.002 | 65.131 | 102.964 | 1.00 | 32.36 |
| ATOM | 6377 | CG2 | VAL | B | 78 | 51.968 | 64.185 | 101.960 | 1.00 | 35.58 |
| ATOM | 6378 | N | PHE | B | 79 | 56.279 | 62.973 | 102.678 | 1.00 | 30.94 |
| ATOM | 6379 | CA | PHE | B | 79 | 57.414 | 62.708 | 103.545 | 1.00 | 30.29 |
| ATOM | 6380 | C | PHE | B | 79 | 57.793 | 63.902 | 104.376 | 1.00 | 30.36 |
| ATOM | 6381 | O | PHE | B | 79 | 57.673 | 65.025 | 103.911 | 1.00 | 30.80 |
| ATOM | 6382 | CB | PHE | B | 79 | 58.618 | 62.302 | 102.715 | 1.00 | 30.23 |
| ATOM | 6383 | CG | PHE | B | 79 | 58.592 | 60.885 | 102.308 | 1.00 | 30.96 |
| ATOM | 6384 | CD1 | PHE | B | 79 | 57.732 | 60.454 | 101.311 | 1.00 | 29.15 |
| ATOM | 6385 | CD2 | PHE | B | 79 | 59.397 | 59.937 | 102.960 | 1.00 | 29.20 |
| ATOM | 6386 | CE1 | PHE | B | 79 | 57.723 | 59.120 | 100.964 | 1.00 | 30.10 |
| ATOM | 6387 | CE2 | PHE | B | 79 | 59.382 | 58.639 | 102.614 | 1.00 | 25.52 |
| ATOM | 6388 | CZ | PHE | B | 79 | 58.554 | 58.211 | 101.632 | 1.00 | 25.12 |
| ATOM | 6389 | N | ASN | B | 80 | 58.255 | 63.657 | 105.598 | 1.00 | 29.03 |
| ATOM | 6390 | CA | ASN | B | 80 | 58.741 | 64.704 | 106.465 | 1.00 | 29.19 |
| ATOM | 6391 | C | ASN | B | 80 | 60.256 | 64.705 | 106.318 | 1.00 | 29.44 |
| ATOM | 6392 | O | ASN | B | 80 | 60.900 | 63.712 | 106.556 | 1.00 | 30.11 |
| ATOM | 6393 | CB | ASN | B | 80 | 58.296 | 64.431 | 107.898 | 1.00 | 28.74 |
| ATOM | 6394 | CG | ASN | B | 80 | 58.948 | 65.335 | 108.888 | 1.00 | 29.09 |
| ATOM | 6395 | OD1 | ASN | B | 80 | 60.147 | 65.268 | 109.094 | 1.00 | 29.35 |
| ATOM | 6396 | ND2 | ASN | B | 80 | 58.157 | 66.173 | 109.536 | 1.00 | 25.89 |
| ATOM | 6397 | N | ALA | B | 81 | 60.821 | 65.817 | 105.904 | 1.00 | 29.51 |
| ATOM | 6398 | CA | ALA | B | 81 | 62.222 | 65.853 | 105.590 | 1.00 | 30.69 |
| ATOM | 6399 | C | ALA | B | 81 | 63.150 | 65.623 | 106.792 | 1.00 | 30.80 |
| ATOM | 6400 | O | ALA | B | 81 | 64.079 | 64.840 | 106.699 | 1.00 | 30.60 |
| ATOM | 6401 | CB | ALA | B | 81 | 62.563 | 67.148 | 104.873 | 1.00 | 29.54 |
| ATOM | 6402 | N | GLU | B | 82 | 62.895 | 66.310 | 107.895 | 1.00 | 31.58 |
| ATOM | 6403 | CA | GLU | B | 82 | 63.749 | 66.231 | 109.069 | 1.00 | 32.74 |
| ATOM | 6404 | C | GLU | B | 82 | 63.857 | 64.795 | 109.558 | 1.00 | 32.27 |
| ATOM | 6405 | O | GLU | B | 82 | 64.960 | 64.303 | 109.744 | 1.00 | 32.51 |
| ATOM | 6406 | CB | GLU | B | 82 | 63.221 | 67.083 | 110.215 | 1.00 | 33.43 |
| ATOM | 6407 | CG | GLU | B | 82 | 64.199 | 67.164 | 111.383 | 1.00 | 36.43 |
| ATOM | 6408 | CD | GLU | B | 82 | 65.576 | 67.642 | 110.968 | 1.00 | 40.84 |
| ATOM | 6409 | OE1 | GLU | B | 82 | 65.677 | 68.473 | 110.041 | 1.00 | 44.66 |
| ATOM | 6410 | OE2 | GLU | B | 82 | 66.566 | 67.193 | 111.557 | 1.00 | 43.16 |
| ATOM | 6411 | N | TYR | B | 83 | 62.717 | 64.129 | 109.709 | 1.00 | 30.92 |
| ATOM | 6412 | CA | TYR | B | 83 | 62.684 | 62.779 | 110.283 | 1.00 | 30.84 |
| ATOM | 6413 | C | TYR | B | 83 | 62.452 | 61.596 | 109.367 | 1.00 | 30.26 |
| ATOM | 6414 | O | TYR | B | 83 | 62.676 | 60.488 | 109.780 | 1.00 | 29.50 |
| ATOM | 6415 | CB | TYR | B | 83 | 61.628 | 62.731 | 111.371 | 1.00 | 29.67 |
| ATOM | 6416 | CG | TYR | B | 83 | 61.864 | 63.793 | 112.364 | 1.00 | 30.76 |
| ATOM | 6417 | CD1 | TYR | B | 83 | 62.976 | 63.736 | 113.187 | 1.00 | 32.90 |
| ATOM | 6418 | CD2 | TYR | B | 83 | 61.007 | 64.870 | 112.488 | 1.00 | 29.61 |
| ATOM | 6419 | CE1 | TYR | B | 83 | 63.228 | 64.728 | 114.126 | 1.00 | 32.38 |
| ATOM | 6420 | CE2 | TYR | B | 83 | 61.244 | 65.857 | 113.414 | 1.00 | 32.14 |
| ATOM | 6421 | CZ | TYR | B | 83 | 62.379 | 65.786 | 114.221 | 1.00 | 34.48 |
| ATOM | 6422 | OH | TYR | B | 83 | 62.650 | 66.746 | 115.181 | 1.00 | 40.18 |
| ATOM | 6423 | N | GLY | B | 84 | 61.971 | 61.788 | 108.149 | 1.00 | 30.54 |
| ATOM | 6424 | CA | GLY | B | 84 | 61.819 | 60.641 | 107.265 | 1.00 | 30.76 |
| ATOM | 6425 | C | GLY | B | 84 | 60.520 | 59.863 | 107.382 | 1.00 | 30.75 |
| ATOM | 6426 | O | GLY | B | 84 | 60.257 | 58.998 | 106.549 | 1.00 | 31.47 |
| ATOM | 6427 | N | ASN | B | 85 | 59.691 | 60.138 | 108.387 | 1.00 | 31.23 |
| ATOM | 6428 | CA | ASN | B | 85 | 58.399 | 59.446 | 108.443 | 1.00 | 31.65 |
| ATOM | 6429 | C | ASN | B | 85 | 57.477 | 59.915 | 107.279 | 1.00 | 32.17 |
| ATOM | 6430 | O | ASN | B | 85 | 57.537 | 61.063 | 106.852 | 1.00 | 30.51 |
| ATOM | 6431 | CB | ASN | B | 85 | 57.699 | 59.607 | 109.803 | 1.00 | 30.47 |
| ATOM | 6432 | CG | ASN | B | 85 | 57.315 | 61.007 | 110.089 | 1.00 | 31.43 |
| ATOM | 6433 | OD1 | ASN | B | 85 | 58.191 | 61.876 | 110.191 | 1.00 | 33.75 |
| ATOM | 6434 | ND2 | ASN | B | 85 | 56.000 | 61.269 | 110.198 | 1.00 | 32.85 |
| ATOM | 6435 | N | SER | B | 86 | 56.628 | 59.017 | 106.810 | 1.00 | 33.47 |
| ATOM | 6436 | CA | SER | B | 86 | 55.761 | 59.280 | 105.674 | 1.00 | 35.12 |
| ATOM | 6437 | C | SER | B | 86 | 54.369 | 58.849 | 105.986 | 1.00 | 36.58 |
| ATOM | 6438 | O | SER | B | 86 | 54.146 | 58.032 | 106.877 | 1.00 | 36.15 |
| ATOM | 6439 | CB | SER | B | 86 | 56.200 | 58.460 | 104.490 | 1.00 | 35.25 |
| ATOM | 6440 | OG | SER | B | 86 | 56.483 | 57.165 | 104.932 | 1.00 | 37.10 |
| ATOM | 6441 | N | SER | B | 87 | 53.430 | 59.419 | 105.242 | 1.00 | 37.64 |
| ATOM | 6442 | CA | SER | B | 87 | 52.028 | 59.089 | 105.364 | 1.00 | 38.75 |
| ATOM | 6443 | C | SER | B | 87 | 51.394 | 59.084 | 103.977 | 1.00 | 39.14 |
| ATOM | 6444 | O | SER | B | 87 | 51.930 | 59.651 | 103.026 | 1.00 | 39.55 |
| ATOM | 6445 | CB | SER | B | 87 | 51.290 | 60.079 | 106.264 | 1.00 | 39.10 |
| ATOM | 6446 | OG | SER | B | 87 | 51.755 | 59.996 | 107.591 | 1.00 | 40.45 |
| ATOM | 6447 | N | VAL | B | 88 | 50.239 | 58.434 | 103.889 | 1.00 | 39.63 |
| ATOM | 6448 | CA | VAL | B | 88 | 49.509 | 58.296 | 102.643 | 1.00 | 40.87 |
| ATOM | 6449 | C | VAL | B | 88 | 48.643 | 59.539 | 102.386 | 1.00 | 41.23 |
| ATOM | 6450 | O | VAL | B | 88 | 47.661 | 59.782 | 103.086 | 1.00 | 40.79 |
| ATOM | 6451 | CB | VAL | B | 88 | 48.631 | 57.004 | 102.636 | 1.00 | 41.06 |
| ATOM | 6452 | CG1 | VAL | B | 88 | 48.060 | 56.712 | 104.036 | 1.00 | 43.96 |
| ATOM | 6453 | CG2 | VAL | B | 88 | 47.525 | 57.135 | 101.592 | 1.00 | 40.56 |
| ATOM | 6454 | N | PHE | B | 89 | 49.022 | 60.312 | 101.378 | 1.00 | 42.05 |
| ATOM | 6455 | CA | PHE | B | 89 | 48.214 | 61.446 | 100.964 | 1.00 | 43.87 |
| ATOM | 6456 | C | PHE | B | 89 | 46.989 | 61.027 | 100.163 | 1.00 | 44.21 |
| ATOM | 6457 | O | PHE | B | 89 | 45.917 | 61.555 | 100.345 | 1.00 | 43.68 |
| ATOM | 6458 | CB | PHE | B | 89 | 49.002 | 62.442 | 100.126 | 1.00 | 44.25 |
| ATOM | 6459 | CG | PHE | B | 89 | 48.279 | 63.735 | 99.959 | 1.00 | 46.62 |
| ATOM | 6460 | CD1 | PHE | B | 89 | 48.319 | 64.690 | 100.960 | 1.00 | 49.83 |
| ATOM | 6461 | CD2 | PHE | B | 89 | 47.494 | 63.967 | 98.849 | 1.00 | 48.66 |
| ATOM | 6462 | CE1 | PHE | B | 89 | 47.617 | 65.874 | 100.831 | 1.00 | 50.73 |
| ATOM | 6463 | CE2 | PHE | B | 89 | 46.785 | 65.136 | 98.725 | 1.00 | 49.25 |
| ATOM | 6464 | CZ | PHE | B | 89 | 46.848 | 66.088 | 99.719 | 1.00 | 50.32 |
| ATOM | 6465 | N | LEU | B | 90 | 47.173 | 60.072 | 99.268 | 1.00 | 45.65 |
| ATOM | 6466 | CA | LEU | B | 90 | 46.098 | 59.577 | 98.434 | 1.00 | 47.32 |
| ATOM | 6467 | C | LEU | B | 90 | 46.329 | 58.093 | 98.306 | 1.00 | 48.48 |
| ATOM | 6468 | O | LEU | B | 90 | 47.341 | 57.680 | 97.752 | 1.00 | 47.19 |
| ATOM | 6469 | CB | LEU | B | 90 | 46.192 | 60.220 | 97.050 | 1.00 | 47.51 |
| ATOM | 6470 | CG | LEU | B | 90 | 44.961 | 60.573 | 96.237 | 1.00 | 49.43 |
| ATOM | 6471 | CD1 | LEU | B | 90 | 45.201 | 60.128 | 94.783 | 1.00 | 50.89 |
| ATOM | 6472 | CD2 | LEU | B | 90 | 43.731 | 59.945 | 96.784 | 1.00 | 50.36 |
| ATOM | 6473 | N | GLU | B | 91 | 45.387 | 57.302 | 98.804 | 1.00 | 50.77 |
| ATOM | 6474 | CA | GLU | B | 91 | 45.523 | 55.845 | 98.815 | 1.00 | 53.33 |
| ATOM | 6475 | C | GLU | B | 91 | 45.681 | 55.245 | 97.410 | 1.00 | 54.61 |
| ATOM | 6476 | O | GLU | B | 91 | 44.958 | 55.617 | 96.503 | 1.00 | 53.67 |
| ATOM | 6477 | CB | GLU | B | 91 | 44.312 | 55.185 | 99.494 | 1.00 | 53.90 |
| ATOM | 6478 | CG | GLU | B | 91 | 43.496 | 56.067 | 100.445 | 1.00 | 56.83 |
| ATOM | 6479 | CD | GLU | B | 91 | 42.375 | 56.850 | 99.752 | 1.00 | 60.34 |
| ATOM | 6480 | OE1 | GLU | B | 91 | 42.639 | 57.978 | 99.252 | 1.00 | 59.61 |
| ATOM | 6481 | OE2 | GLU | B | 91 | 41.226 | 56.325 | 99.707 | 1.00 | 62.58 |
| ATOM | 6482 | N | ASN | B | 92 | 46.617 | 54.305 | 97.244 | 1.00 | 56.76 |
| ATOM | 6483 | CA | ASN | B | 92 | 46.790 | 53.626 | 95.957 | 1.00 | 58.94 |
| ATOM | 6484 | C | ASN | B | 92 | 45.440 | 53.196 | 95.373 | 1.00 | 60.58 |
| ATOM | 6485 | O | ASN | B | 92 | 45.181 | 53.331 | 94.167 | 1.00 | 60.40 |
| ATOM | 6486 | CB | ASN | B | 92 | 47.755 | 52.412 | 96.063 | 1.00 | 58.98 |
| ATOM | 6487 | CG | ASN | B | 92 | 47.259 | 51.311 | 97.022 | 1.00 | 59.52 |
| ATOM | 6488 | OD1 | ASN | B | 92 | 46.362 | 51.542 | 97.840 | 1.00 | 60.59 |
| ATOM | 6489 | ND2 | ASN | B | 92 | 47.817 | 50.094 | 96.897 | 1.00 | 61.81 |
| ATOM | 6490 | N | SER | B | 93 | 44.580 | 52.720 | 96.264 | 1.00 | 62.60 |
| ATOM | 6491 | CA | SER | B | 93 | 43.268 | 52.168 | 95.922 | 1.00 | 64.67 |
| ATOM | 6492 | C | SER | B | 93 | 42.282 | 53.169 | 95.329 | 1.00 | 65.69 |
| ATOM | 6493 | O | SER | B | 93 | 41.597 | 52.858 | 94.344 | 1.00 | 66.09 |
| ATOM | 6494 | CB | SER | B | 93 | 42.659 | 51.570 | 97.187 | 1.00 | 64.77 |
| ATOM | 6495 | OG | SER | B | 93 | 43.699 | 51.255 | 98.109 | 1.00 | 66.81 |
| ATOM | 6496 | N | THR | B | 94 | 42.182 | 54.349 | 95.943 | 1.00 | 66.89 |
| ATOM | 6497 | CA | THR | B | 94 | 41.285 | 55.377 | 95.440 | 1.00 | 67.90 |
| ATOM | 6498 | C | THR | B | 94 | 41.516 | 55.453 | 93.950 | 1.00 | 68.67 |
| ATOM | 6499 | O | THR | B | 94 | 42.652 | 55.387 | 93.481 | 1.00 | 68.63 |
| ATOM | 6500 | CB | THR | B | 94 | 41.571 | 56.749 | 96.064 | 1.00 | 68.14 |
| ATOM | 6501 | OG1 | THR | B | 94 | 42.354 | 56.609 | 97.254 | 1.00 | 67.83 |
| ATOM | 6502 | CG2 | THR | B | 94 | 40.274 | 57.410 | 96.532 | 1.00 | 68.08 |
| ATOM | 6503 | N | PHE | B | 95 | 40.430 | 55.583 | 93.207 | 1.00 | 69.89 |
| ATOM | 6504 | CA | PHE | B | 95 | 40.497 | 55.602 | 91.754 | 1.00 | 70.65 |
| ATOM | 6505 | C | PHE | B | 95 | 40.944 | 54.252 | 91.233 | 1.00 | 71.25 |
| ATOM | 6506 | O | PHE | B | 95 | 42.029 | 54.099 | 90.678 | 1.00 | 71.18 |
| ATOM | 6507 | CB | PHE | B | 95 | 41.392 | 56.735 | 91.256 | 1.00 | 70.73 |
| ATOM | 6508 | CG | PHE | B | 95 | 41.049 | 58.049 | 91.865 | 1.00 | 70.42 |
| ATOM | 6509 | CD1 | PHE | B | 95 | 39.734 | 58.469 | 91.910 | 1.00 | 70.08 |
| ATOM | 6510 | CD2 | PHE | B | 95 | 42.025 | 58.840 | 92.435 | 1.00 | 70.08 |
| ATOM | 6511 | CE1 | PHE | B | 95 | 39.403 | 59.654 | 92.493 | 1.00 | 69.12 |
| ATOM | 6512 | CE2 | PHE | B | 95 | 41.691 | 60.030 | 93.014 | 1.00 | 70.02 |
| ATOM | 6513 | CZ | PHE | B | 95 | 40.376 | 60.433 | 93.041 | 1.00 | 69.12 |
| ATOM | 6514 | N | ASP | B | 96 | 40.085 | 53.276 | 91.501 | 1.00 | 71.95 |
| ATOM | 6515 | CA | ASP | B | 96 | 40.174 | 51.930 | 90.968 | 1.00 | 72.26 |
| ATOM | 6516 | C | ASP | B | 96 | 38.920 | 51.861 | 90.104 | 1.00 | 72.48 |
| ATOM | 6517 | O | ASP | B | 96 | 38.931 | 51.431 | 88.940 | 1.00 | 72.15 |
| ATOM | 6518 | CB | ASP | B | 96 | 40.089 | 50.885 | 92.094 | 1.00 | 72.41 |
| ATOM | 6519 | CG | ASP | B | 96 | 41.452 | 50.303 | 92.491 | 1.00 | 72.76 |
| ATOM | 6520 | OD1 | ASP | B | 96 | 42.461 | 50.610 | 91.830 | 1.00 | 74.22 |
| ATOM | 6521 | OD2 | ASP | B | 96 | 41.606 | 49.509 | 93.450 | 1.00 | 71.03 |
| ATOM | 6522 | N | GLU | B | 97 | 37.831 | 52.337 | 90.701 | 1.00 | 72.66 |
| ATOM | 6523 | CA | GLU | B | 97 | 36.521 | 52.362 | 90.067 | 1.00 | 72.60 |
| ATOM | 6524 | C | GLU | B | 97 | 36.261 | 53.683 | 89.321 | 1.00 | 71.81 |
| ATOM | 6525 | O | GLU | B | 97 | 35.142 | 53.933 | 88.872 | 1.00 | 71.85 |
| ATOM | 6526 | CB | GLU | B | 97 | 35.486 | 52.159 | 91.167 | 1.00 | 73.02 |
| ATOM | 6527 | CG | GLU | B | 97 | 34.042 | 52.011 | 90.723 | 1.00 | 74.39 |
| ATOM | 6528 | CD | GLU | B | 97 | 33.130 | 51.759 | 91.910 | 1.00 | 76.22 |
| ATOM | 6529 | OE1 | GLU | B | 97 | 33.579 | 52.004 | 93.059 | 1.00 | 76.78 |
| ATOM | 6530 | OE2 | GLU | B | 97 | 31.979 | 51.313 | 91.696 | 1.00 | 76.84 |
| ATOM | 6531 | N | PHE | B | 98 | 37.294 | 54.518 | 89.175 | 1.00 | 70.65 |
| ATOM | 6532 | CA | PHE | B | 98 | 37.139 | 55.807 | 88.505 | 1.00 | 69.57 |
| ATOM | 6533 | C | PHE | B | 98 | 36.692 | 55.642 | 87.051 | 1.00 | 68.08 |
| ATOM | 6534 | O | PHE | B | 98 | 35.986 | 56.486 | 86.522 | 1.00 | 68.19 |
| ATOM | 6535 | CB | PHE | B | 98 | 38.429 | 56.624 | 88.577 | 1.00 | 69.94 |
| ATOM | 6536 | CG | PHE | B | 98 | 38.381 | 57.894 | 87.772 | 1.00 | 70.49 |
| ATOM | 6537 | CD1 | PHE | B | 98 | 37.458 | 58.887 | 88.073 | 1.00 | 70.34 |
| ATOM | 6538 | CD2 | PHE | B | 98 | 39.246 | 58.088 | 86.701 | 1.00 | 71.59 |
| ATOM | 6539 | CE1 | PHE | B | 98 | 37.411 | 60.063 | 87.333 | 1.00 | 70.63 |
| ATOM | 6540 | CE2 | PHE | B | 98 | 39.201 | 59.267 | 85.947 | 1.00 | 71.72 |
| ATOM | 6541 | CZ | PHE | B | 98 | 38.281 | 60.255 | 86.270 | 1.00 | 71.03 |
| ATOM | 6542 | N | GLY | B | 99 | 37.099 | 54.564 | 86.398 | 1.00 | 66.41 |
| ATOM | 6543 | CA | GLY | B | 99 | 36.599 | 54.293 | 85.060 | 1.00 | 65.26 |
| ATOM | 6544 | C | GLY | B | 99 | 37.471 | 54.761 | 83.913 | 1.00 | 63.94 |
| ATOM | 6545 | O | GLY | B | 99 | 37.077 | 54.635 | 82.743 | 1.00 | 63.67 |
| ATOM | 6546 | N | HIS | B | 100 | 38.636 | 55.311 | 84.252 | 1.00 | 62.08 |
| ATOM | 6547 | CA | HIS | B | 100 | 39.618 | 55.740 | 83.269 | 1.00 | 60.54 |
| ATOM | 6548 | C | HIS | B | 100 | 41.006 | 55.611 | 83.858 | 1.00 | 58.93 |
| ATOM | 6549 | O | HIS | B | 100 | 41.216 | 55.858 | 85.042 | 1.00 | 58.09 |
| ATOM | 6550 | CB | HIS | B | 100 | 39.495 | 57.227 | 82.924 | 1.00 | 60.44 |
| ATOM | 6551 | CG | HIS | B | 100 | 38.131 | 57.681 | 82.520 | 1.00 | 60.43 |
| ATOM | 6552 | ND1 | HIS | B | 100 | 37.367 | 58.516 | 83.309 | 1.00 | 61.86 |
| ATOM | 6553 | CD2 | HIS | B | 100 | 37.435 | 57.506 | 81.373 | 1.00 | 61.34 |
| ATOM | 6554 | CE1 | HIS | B | 100 | 36.236 | 58.795 | 82.682 | 1.00 | 61.63 |
| ATOM | 6555 | NE2 | HIS | B | 100 | 36.252 | 58.195 | 81.505 | 1.00 | 60.88 |
| ATOM | 6556 | N | SER | B | 101 | 41.968 | 55.264 | 83.019 | 1.00 | 57.46 |
| ATOM | 6557 | CA | SER | B | 101 | 43.352 | 55.333 | 83.427 | 1.00 | 56.47 |
| ATOM | 6558 | C | SER | B | 101 | 43.620 | 56.813 | 83.736 | 1.00 | 55.22 |
| ATOM | 6559 | O | SER | B | 101 | 43.280 | 57.678 | 82.930 | 1.00 | 55.25 |
| ATOM | 6560 | CB | SER | B | 101 | 44.261 | 54.834 | 82.300 | 1.00 | 56.82 |
| ATOM | 6561 | OG | SER | B | 101 | 45.485 | 55.565 | 82.254 | 1.00 | 57.96 |
| ATOM | 6562 | N | ILE | B | 102 | 44.170 | 57.103 | 84.912 | 1.00 | 53.65 |
| ATOM | 6563 | CA | ILE | B | 102 | 44.558 | 58.455 | 85.274 | 1.00 | 52.71 |
| ATOM | 6564 | C | ILE | B | 102 | 46.024 | 58.646 | 84.939 | 1.00 | 51.90 |
| ATOM | 6565 | O | ILE | B | 102 | 46.891 | 57.906 | 85.408 | 1.00 | 51.45 |
| ATOM | 6566 | CB | ILE | B | 102 | 44.352 | 58.732 | 86.761 | 1.00 | 52.96 |
| ATOM | 6567 | CG1 | ILE | B | 102 | 42.889 | 59.040 | 87.048 | 1.00 | 53.56 |
| ATOM | 6568 | CG2 | ILE | B | 102 | 45.213 | 59.930 | 87.210 | 1.00 | 52.05 |
| ATOM | 6569 | CD1 | ILE | B | 102 | 42.546 | 58.864 | 88.494 | 1.00 | 54.21 |
| ATOM | 6570 | N | ASN | B | 103 | 46.303 | 59.684 | 84.172 | 1.00 | 50.68 |
| ATOM | 6571 | CA | ASN | B | 103 | 47.633 | 59.886 | 83.621 | 1.00 | 50.19 |
| ATOM | 6572 | C | ASN | B | 103 | 48.569 | 60.727 | 84.493 | 1.00 | 49.33 |
| ATOM | 6573 | O | ASN | B | 103 | 49.776 | 60.549 | 84.451 | 1.00 | 48.89 |
| ATOM | 6574 | CB | ASN | B | 103 | 47.449 | 60.494 | 82.230 | 1.00 | 50.12 |
| ATOM | 6575 | CG | ASN | B | 103 | 48.722 | 60.935 | 81.606 | 1.00 | 50.68 |
| ATOM | 6576 | OD1 | ASN | B | 103 | 49.186 | 62.042 | 81.864 | 1.00 | 52.56 |
| ATOM | 6577 | ND2 | ASN | B | 103 | 49.272 | 60.106 | 80.721 | 1.00 | 48.56 |
| ATOM | 6578 | N | ASP | B | 104 | 48.018 | 61.627 | 85.291 | 1.00 | 48.67 |
| ATOM | 6579 | CA | ASP | B | 104 | 48.843 | 62.489 | 86.129 | 1.00 | 48.99 |
| ATOM | 6580 | C | ASP | B | 104 | 47.926 | 63.154 | 87.166 | 1.00 | 48.52 |
| ATOM | 6581 | O | ASP | B | 104 | 46.720 | 62.911 | 87.189 | 1.00 | 48.70 |
| ATOM | 6582 | CB | ASP | B | 104 | 49.593 | 63.515 | 85.258 | 1.00 | 49.21 |
| ATOM | 6583 | CG | ASP | B | 104 | 50.831 | 64.116 | 85.942 | 1.00 | 51.02 |
| ATOM | 6584 | OD1 | ASP | B | 104 | 50.886 | 64.220 | 87.192 | 1.00 | 57.49 |
| ATOM | 6585 | OD2 | ASP | B | 104 | 51.809 | 64.550 | 85.311 | 1.00 | 51.76 |
| ATOM | 6586 | N | TYR | B | 105 | 48.473 | 63.996 | 88.022 | 1.00 | 48.07 |
| ATOM | 6587 | CA | TYR | B | 105 | 47.669 | 64.584 | 89.072 | 1.00 | 48.29 |
| ATOM | 6588 | C | TYR | B | 105 | 48.363 | 65.835 | 89.531 | 1.00 | 47.82 |
| ATOM | 6589 | O | TYR | B | 105 | 49.553 | 66.008 | 89.297 | 1.00 | 46.92 |
| ATOM | 6590 | CB | TYR | B | 105 | 47.507 | 63.623 | 90.252 | 1.00 | 48.41 |
| ATOM | 6591 | CG | TYR | B | 105 | 48.802 | 63.411 | 90.975 | 1.00 | 50.88 |
| ATOM | 6592 | CD1 | TYR | B | 105 | 49.285 | 64.366 | 91.845 | 1.00 | 53.35 |
| ATOM | 6593 | CD2 | TYR | B | 105 | 49.571 | 62.279 | 90.754 | 1.00 | 53.13 |
| ATOM | 6594 | CE1 | TYR | B | 105 | 50.484 | 64.199 | 92.491 | 1.00 | 54.72 |
| ATOM | 6595 | CE2 | TYR | B | 105 | 50.780 | 62.100 | 91.410 | 1.00 | 54.12 |
| ATOM | 6596 | CZ | TYR | B | 105 | 51.229 | 63.071 | 92.277 | 1.00 | 54.90 |
| ATOM | 6597 | OH | TYR | B | 105 | 52.438 | 62.932 | 92.941 | 1.00 | 56.72 |
| ATOM | 6598 | N | SER | B | 106 | 47.607 | 66.712 | 90.179 | 1.00 | 47.41 |
| ATOM | 6599 | CA | SER | B | 106 | 48.154 | 67.975 | 90.634 | 1.00 | 47.19 |
| ATOM | 6600 | C | SER | B | 106 | 47.355 | 68.466 | 91.821 | 1.00 | 47.24 |
| ATOM | 6601 | O | SER | B | 106 | 46.183 | 68.832 | 91.712 | 1.00 | 46.40 |
| ATOM | 6602 | CB | SER | B | 106 | 48.134 | 69.020 | 89.533 | 1.00 | 47.09 |
| ATOM | 6603 | OG | SER | B | 106 | 48.471 | 70.278 | 90.078 | 1.00 | 47.17 |
| ATOM | 6604 | N | ILE | B | 107 | 48.030 | 68.507 | 92.955 | 1.00 | 47.54 |
| ATOM | 6605 | CA | ILE | B | 107 | 47.364 | 68.782 | 94.201 | 1.00 | 47.97 |
| ATOM | 6606 | C | ILE | B | 107 | 47.408 | 70.258 | 94.453 | 1.00 | 47.17 |
| ATOM | 6607 | O | ILE | B | 107 | 48.403 | 70.897 | 94.208 | 1.00 | 46.88 |
| ATOM | 6608 | CB | ILE | B | 107 | 48.023 | 67.952 | 95.315 | 1.00 | 48.46 |
| ATOM | 6609 | CG1 | ILE | B | 107 | 48.132 | 66.494 | 94.834 | 1.00 | 50.38 |
| ATOM | 6610 | CG2 | ILE | B | 107 | 47.221 | 68.028 | 96.586 | 1.00 | 48.95 |
| ATOM | 6611 | CD1 | ILE | B | 107 | 48.792 | 65.516 | 95.824 | 1.00 | 51.53 |
| ATOM | 6612 | N | SER | B | 108 | 46.280 | 70.797 | 94.874 | 1.00 | 46.98 |
| ATOM | 6613 | CA | SER | B | 108 | 46.165 | 72.198 | 95.182 | 1.00 | 47.44 |
| ATOM | 6614 | C | SER | B | 108 | 47.139 | 72.506 | 96.299 | 1.00 | 47.37 |
| ATOM | 6615 | O | SER | B | 108 | 47.360 | 71.655 | 97.148 | 1.00 | 47.37 |
| ATOM | 6616 | CB | SER | B | 108 | 44.750 | 72.479 | 95.637 | 1.00 | 47.48 |
| ATOM | 6617 | OG | SER | B | 108 | 43.908 | 72.555 | 94.511 | 1.00 | 50.87 |
| ATOM | 6618 | N | PRO | B | 109 | 47.680 | 73.714 | 96.314 | 1.00 | 47.60 |
| ATOM | 6619 | CA | PRO | B | 109 | 48.726 | 74.098 | 97.270 | 1.00 | 48.07 |
| ATOM | 6620 | C | PRO | B | 109 | 48.248 | 74.084 | 98.701 | 1.00 | 48.47 |
| ATOM | 6621 | O | PRO | B | 109 | 48.884 | 73.537 | 99.582 | 1.00 | 48.42 |
| ATOM | 6622 | CB | PRO | B | 109 | 49.067 | 75.550 | 96.896 | 1.00 | 48.14 |
| ATOM | 6623 | CG | PRO | B | 109 | 48.258 | 75.909 | 95.689 | 1.00 | 48.80 |
| ATOM | 6624 | CD | PRO | B | 109 | 47.293 | 74.812 | 95.418 | 1.00 | 48.07 |
| ATOM | 6625 | N | ASP | B | 110 | 47.118 | 74.732 | 98.915 | 1.00 | 48.72 |
| ATOM | 6626 | CA | ASP | B | 110 | 46.524 | 74.837 | 100.222 | 1.00 | 48.24 |
| ATOM | 6627 | C | ASP | B | 110 | 45.494 | 73.728 | 100.067 | 1.00 | 48.77 |
| ATOM | 6628 | O | ASP | B | 110 | 44.331 | 73.942 | 99.609 | 1.00 | 49.67 |
| ATOM | 6629 | CB | ASP | B | 110 | 46.040 | 76.281 | 100.413 | 1.00 | 47.85 |
| ATOM | 6630 | CG | ASP | B | 110 | 47.234 | 77.315 | 100.391 | 1.00 | 45.51 |
| ATOM | 6631 | OD1 | ASP | B | 110 | 48.376 | 76.906 | 100.255 | 1.00 | 41.05 |
| ATOM | 6632 | OD2 | ASP | B | 110 | 47.150 | 78.556 | 100.515 | 1.00 | 45.53 |
| ATOM | 6633 | N | GLY | B | 111 | 46.028 | 72.549 | 100.427 | 1.00 | 48.67 |
| ATOM | 6634 | CA | GLY | B | 111 | 45.611 | 71.174 | 100.106 | 1.00 | 47.96 |
| ATOM | 6635 | C | GLY | B | 111 | 44.234 | 70.538 | 100.027 | 1.00 | 47.65 |
| ATOM | 6636 | O | GLY | B | 111 | 44.157 | 69.318 | 100.186 | 1.00 | 48.21 |
| ATOM | 6637 | N | GLN | B | 112 | 43.191 | 71.280 | 99.681 | 1.00 | 46.73 |
| ATOM | 6638 | CA | GLN | B | 112 | 41.828 | 70.757 | 99.688 | 1.00 | 46.55 |
| ATOM | 6639 | C | GLN | B | 112 | 41.368 | 69.958 | 98.453 | 1.00 | 45.92 |
| ATOM | 6640 | O | GLN | B | 112 | 40.404 | 69.189 | 98.507 | 1.00 | 45.81 |
| ATOM | 6641 | CB | GLN | B | 112 | 40.891 | 71.939 | 99.910 | 1.00 | 47.23 |
| ATOM | 6642 | CG | GLN | B | 112 | 41.335 | 72.836 | 101.085 | 1.00 | 48.40 |
| ATOM | 6643 | CD | GLN | B | 112 | 40.268 | 73.776 | 101.521 | 1.00 | 50.36 |
| ATOM | 6644 | OE1 | GLN | B | 112 | 39.258 | 73.927 | 100.840 | 1.00 | 51.07 |
| ATOM | 6645 | NE2 | GLN | B | 112 | 40.464 | 74.407 | 102.671 | 1.00 | 52.21 |
| ATOM | 6646 | N | PHE | B | 113 | 42.057 | 70.122 | 97.334 | 1.00 | 45.08 |
| ATOM | 6647 | CA | PHE | B | 113 | 41.642 | 69.458 | 96.113 | 1.00 | 43.92 |
| ATOM | 6648 | C | PHE | B | 113 | 42.805 | 68.847 | 95.343 | 1.00 | 42.79 |
| ATOM | 6649 | O | PHE | B | 113 | 43.958 | 69.219 | 95.512 | 1.00 | 42.89 |
| ATOM | 6650 | CB | PHE | B | 113 | 40.929 | 70.466 | 95.214 | 1.00 | 43.63 |
| ATOM | 6651 | CG | PHE | B | 113 | 39.750 | 71.099 | 95.848 | 1.00 | 42.05 |
| ATOM | 6652 | CD1 | PHE | B | 113 | 38.488 | 70.569 | 95.677 | 1.00 | 42.72 |
| ATOM | 6653 | CD2 | PHE | B | 113 | 39.889 | 72.226 | 96.606 | 1.00 | 39.78 |
| ATOM | 6654 | CE1 | PHE | B | 113 | 37.383 | 71.177 | 96.261 | 1.00 | 42.10 |
| ATOM | 6655 | CE2 | PHE | B | 113 | 38.789 | 72.834 | 97.196 | 1.00 | 40.25 |
| ATOM | 6656 | CZ | PHE | B | 113 | 37.554 | 72.322 | 97.031 | 1.00 | 41.31 |
| ATOM | 6657 | N | ILE | B | 114 | 42.487 | 67.880 | 94.505 | 1.00 | 42.17 |
| ATOM | 6658 | CA | ILE | B | 114 | 43.473 | 67.356 | 93.598 | 1.00 | 41.68 |
| ATOM | 6659 | C | ILE | B | 114 | 42.892 | 67.268 | 92.197 | 1.00 | 40.71 |
| ATOM | 6660 | O | ILE | B | 114 | 41.741 | 66.874 | 92.020 | 1.00 | 40.61 |
| ATOM | 6661 | CB | ILE | B | 114 | 43.966 | 65.991 | 94.062 | 1.00 | 41.42 |
| ATOM | 6662 | CG1 | ILE | B | 114 | 45.153 | 65.574 | 93.200 | 1.00 | 41.62 |
| ATOM | 6663 | CG2 | ILE | B | 114 | 42.860 | 64.986 | 93.970 | 1.00 | 41.11 |
| ATOM | 6664 | CD1 | ILE | B | 114 | 45.635 | 64.163 | 93.416 | 1.00 | 42.89 |
| ATOM | 6665 | N | LEU | B | 115 | 43.715 | 67.599 | 91.201 | 1.00 | 39.94 |
| ATOM | 6666 | CA | LEU | B | 115 | 43.320 | 67.535 | 89.789 | 1.00 | 39.10 |
| ATOM | 6667 | C | LEU | B | 115 | 43.642 | 66.161 | 89.210 | 1.00 | 38.76 |
| ATOM | 6668 | O | LEU | B | 115 | 44.777 | 65.737 | 89.259 | 1.00 | 38.30 |
| ATOM | 6669 | CB | LEU | B | 115 | 44.130 | 68.536 | 89.002 | 1.00 | 39.65 |
| ATOM | 6670 | CG | LEU | B | 115 | 43.616 | 69.790 | 88.299 | 1.00 | 40.09 |
| ATOM | 6671 | CD1 | LEU | B | 115 | 44.778 | 70.170 | 87.397 | 1.00 | 40.91 |
| ATOM | 6672 | CD2 | LEU | B | 115 | 42.343 | 69.622 | 87.504 | 1.00 | 38.88 |
| ATOM | 6673 | N | LEU | B | 116 | 42.671 | 65.453 | 88.657 | 1.00 | 38.21 |
| ATOM | 6674 | CA | LEU | B | 116 | 42.982 | 64.185 | 88.024 | 1.00 | 38.17 |
| ATOM | 6675 | C | LEU | B | 116 | 42.968 | 64.422 | 86.527 | 1.00 | 37.63 |
| ATOM | 6676 | O | LEU | B | 116 | 41.992 | 64.913 | 85.989 | 1.00 | 37.08 |
| ATOM | 6677 | CB | LEU | B | 116 | 41.974 | 63.094 | 88.383 | 1.00 | 38.64 |
| ATOM | 6678 | CG | LEU | B | 116 | 41.649 | 62.939 | 89.858 | 1.00 | 39.18 |
| ATOM | 6679 | CD1 | LEU | B | 116 | 40.900 | 61.664 | 90.069 | 1.00 | 41.25 |
| ATOM | 6680 | CD2 | LEU | B | 116 | 42.905 | 62.919 | 90.652 | 1.00 | 39.01 |
| ATOM | 6681 | N | GLU | B | 117 | 44.063 | 64.061 | 85.870 | 1.00 | 37.51 |
| ATOM | 6682 | CA | GLU | B | 117 | 44.234 | 64.281 | 84.444 | 1.00 | 36.48 |
| ATOM | 6683 | C | GLU | B | 117 | 44.052 | 62.967 | 83.754 | 1.00 | 36.12 |
| ATOM | 6684 | O | GLU | B | 117 | 44.751 | 61.994 | 84.054 | 1.00 | 38.07 |
| ATOM | 6685 | CB | GLU | B | 117 | 45.649 | 64.823 | 84.205 | 1.00 | 36.68 |
| ATOM | 6686 | CG | GLU | B | 117 | 45.949 | 65.272 | 82.781 | 1.00 | 35.93 |
| ATOM | 6687 | CD | GLU | B | 117 | 47.387 | 65.728 | 82.593 | 1.00 | 35.27 |
| ATOM | 6688 | OE1 | GLU | B | 117 | 48.193 | 64.901 | 82.173 | 1.00 | 37.75 |
| ATOM | 6689 | OE2 | GLU | B | 117 | 47.718 | 66.905 | 82.825 | 1.00 | 36.09 |
| ATOM | 6690 | N | TYR | B | 118 | 43.138 | 62.902 | 82.817 | 1.00 | 34.75 |
| ATOM | 6691 | CA | TYR | B | 118 | 42.914 | 61.657 | 82.116 | 1.00 | 34.56 |
| ATOM | 6692 | C | TYR | B | 118 | 42.636 | 61.937 | 80.633 | 1.00 | 34.00 |
| ATOM | 6693 | O | TYR | B | 118 | 42.570 | 63.108 | 80.232 | 1.00 | 33.30 |
| ATOM | 6694 | CB | TYR | B | 118 | 41.797 | 60.859 | 82.818 | 1.00 | 34.86 |
| ATOM | 6695 | CG | TYR | B | 118 | 40.404 | 61.439 | 82.742 | 1.00 | 35.02 |
| ATOM | 6696 | CD1 | TYR | B | 118 | 39.990 | 62.450 | 83.598 | 1.00 | 39.23 |
| ATOM | 6697 | CD2 | TYR | B | 118 | 39.500 | 60.948 | 81.844 | 1.00 | 38.13 |
| ATOM | 6698 | CE1 | TYR | B | 118 | 38.677 | 62.981 | 83.529 | 1.00 | 38.18 |
| ATOM | 6699 | CE2 | TYR | B | 118 | 38.209 | 61.457 | 81.753 | 1.00 | 40.19 |
| ATOM | 6700 | CZ | TYR | B | 118 | 37.810 | 62.477 | 82.594 | 1.00 | 40.62 |
| ATOM | 6701 | OH | TYR | B | 118 | 36.538 | 62.959 | 82.462 | 1.00 | 43.07 |
| ATOM | 6702 | N | ASN | B | 119 | 42.518 | 60.876 | 79.834 | 1.00 | 34.57 |
| ATOM | 6703 | CA | ASN | B | 119 | 42.391 | 60.976 | 78.377 | 1.00 | 35.21 |
| ATOM | 6704 | C | ASN | B | 119 | 43.487 | 61.898 | 77.769 | 1.00 | 34.89 |
| ATOM | 6705 | O | ASN | B | 119 | 43.218 | 62.738 | 76.925 | 1.00 | 34.73 |
| ATOM | 6706 | CB | ASN | B | 119 | 41.029 | 61.548 | 77.987 | 1.00 | 35.70 |
| ATOM | 6707 | CG | ASN | B | 119 | 39.910 | 60.521 | 78.017 | 1.00 | 38.47 |
| ATOM | 6708 | OD1 | ASN | B | 119 | 40.140 | 59.326 | 77.869 | 1.00 | 40.47 |
| ATOM | 6709 | ND2 | ASN | B | 119 | 38.673 | 61.004 | 78.174 | 1.00 | 38.75 |
| ATOM | 6710 | N | TYR | B | 120 | 44.705 | 61.798 | 78.255 | 1.00 | 34.18 |
| ATOM | 6711 | CA | TYR | B | 120 | 45.789 | 62.576 | 77.705 | 1.00 | 33.43 |
| ATOM | 6712 | C | TYR | B | 120 | 46.053 | 62.232 | 76.247 | 1.00 | 32.61 |
| ATOM | 6713 | O | TYR | B | 120 | 46.092 | 61.059 | 75.884 | 1.00 | 32.29 |
| ATOM | 6714 | CB | TYR | B | 120 | 47.035 | 62.344 | 78.550 | 1.00 | 33.95 |
| ATOM | 6715 | CG | TYR | B | 120 | 48.380 | 62.689 | 77.930 | 1.00 | 35.78 |
| ATOM | 6716 | CD1 | TYR | B | 120 | 48.990 | 61.846 | 77.019 | 1.00 | 37.16 |
| ATOM | 6717 | CD2 | TYR | B | 120 | 49.081 | 63.814 | 78.349 | 1.00 | 37.19 |
| ATOM | 6718 | CE1 | TYR | B | 120 | 50.269 | 62.157 | 76.500 | 1.00 | 40.14 |
| ATOM | 6719 | CE2 | TYR | B | 120 | 50.331 | 64.116 | 77.855 | 1.00 | 38.09 |
| ATOM | 6720 | CZ | TYR | B | 120 | 50.912 | 63.301 | 76.933 | 1.00 | 39.35 |
| ATOM | 6721 | OH | TYR | B | 120 | 52.140 | 63.654 | 76.448 | 1.00 | 44.06 |
| ATOM | 6722 | N | VAL | B | 121 | 46.154 | 63.266 | 75.412 | 1.00 | 31.03 |
| ATOM | 6723 | CA | VAL | B | 121 | 46.616 | 63.116 | 74.035 | 1.00 | 30.99 |
| ATOM | 6724 | C | VAL | B | 121 | 47.685 | 64.176 | 73.750 | 1.00 | 29.59 |
| ATOM | 6725 | O | VAL | B | 121 | 47.482 | 65.377 | 73.914 | 1.00 | 28.46 |
| ATOM | 6726 | CB | VAL | B | 121 | 45.513 | 63.229 | 73.008 | 1.00 | 30.81 |
| ATOM | 6727 | CG1 | VAL | B | 121 | 46.050 | 62.953 | 71.627 | 1.00 | 33.17 |
| ATOM | 6728 | CG2 | VAL | B | 121 | 44.343 | 62.275 | 73.359 | 1.00 | 33.40 |
| ATOM | 6729 | N | LYS | B | 122 | 48.829 | 63.701 | 73.312 | 1.00 | 29.19 |
| ATOM | 6730 | CA | LYS | B | 122 | 49.961 | 64.558 | 73.126 | 1.00 | 28.96 |
| ATOM | 6731 | C | LYS | B | 122 | 49.876 | 65.317 | 71.807 | 1.00 | 28.45 |
| ATOM | 6732 | O | LYS | B | 122 | 49.398 | 64.788 | 70.791 | 1.00 | 26.56 |
| ATOM | 6733 | CB | LYS | B | 122 | 51.209 | 63.708 | 73.104 | 1.00 | 28.59 |
| ATOM | 6734 | CG | LYS | B | 122 | 52.495 | 64.485 | 72.963 | 1.00 | 30.76 |
| ATOM | 6735 | CD | LYS | B | 122 | 53.699 | 63.494 | 72.795 | 1.00 | 34.96 |
| ATOM | 6736 | CE | LYS | B | 122 | 54.590 | 63.911 | 71.635 | 1.00 | 36.27 |
| ATOM | 6737 | NZ | LYS | B | 122 | 55.583 | 64.866 | 72.057 | 1.00 | 40.16 |
| ATOM | 6738 | N | GLN | B | 123 | 50.389 | 66.538 | 71.849 | 1.00 | 27.65 |
| ATOM | 6739 | CA | GLN | B | 123 | 50.655 | 67.288 | 70.646 | 1.00 | 28.12 |
| ATOM | 6740 | C | GLN | B | 123 | 52.187 | 67.360 | 70.450 | 1.00 | 26.97 |
| ATOM | 6741 | O | GLN | B | 123 | 52.804 | 66.380 | 70.029 | 1.00 | 25.72 |
| ATOM | 6742 | CB | GLN | B | 123 | 50.000 | 68.652 | 70.735 | 1.00 | 28.16 |
| ATOM | 6743 | CG | GLN | B | 123 | 50.059 | 69.399 | 69.435 | 1.00 | 31.73 |
| ATOM | 6744 | CD | GLN | B | 123 | 49.247 | 70.665 | 69.458 | 1.00 | 35.72 |
| ATOM | 6745 | OE1 | GLN | B | 123 | 48.382 | 70.846 | 68.613 | 1.00 | 40.73 |
| ATOM | 6746 | NE2 | GLN | B | 123 | 49.506 | 71.540 | 70.437 | 1.00 | 35.54 |
| ATOM | 6747 | N | TRP | B | 124 | 52.813 | 68.495 | 70.766 | 1.00 | 26.13 |
| ATOM | 6748 | CA | TRP | B | 124 | 54.250 | 68.615 | 70.569 | 1.00 | 24.86 |
| ATOM | 6749 | C | TRP | B | 124 | 55.074 | 68.167 | 71.783 | 1.00 | 25.01 |
| ATOM | 6750 | O | TRP | B | 124 | 54.705 | 67.197 | 72.495 | 1.00 | 23.66 |
| ATOM | 6751 | CB | TRP | B | 124 | 54.606 | 70.023 | 70.089 | 1.00 | 25.47 |
| ATOM | 6752 | CG | TRP | B | 124 | 53.657 | 70.539 | 69.053 | 1.00 | 24.48 |
| ATOM | 6753 | CD1 | TRP | B | 124 | 52.942 | 71.705 | 69.104 | 1.00 | 24.28 |
| ATOM | 6754 | CD2 | TRP | B | 124 | 53.300 | 69.900 | 67.794 | 1.00 | 24.34 |
| ATOM | 6755 | NE1 | TRP | B | 124 | 52.157 | 71.823 | 67.976 | 1.00 | 25.29 |
| ATOM | 6756 | CE2 | TRP | B | 124 | 52.357 | 70.741 | 67.153 | 1.00 | 23.47 |
| ATOM | 6757 | CE3 | TRP | B | 124 | 53.667 | 68.703 | 67.171 | 1.00 | 21.99 |
| ATOM | 6758 | CZ2 | TRP | B | 124 | 51.757 | 70.420 | 65.934 | 1.00 | 21.90 |
| ATOM | 6759 | CZ3 | TRP | B | 124 | 53.076 | 68.371 | 65.936 | 1.00 | 22.42 |
| ATOM | 6760 | CH2 | TRP | B | 124 | 52.144 | 69.250 | 65.326 | 1.00 | 24.29 |
| ATOM | 6761 | N | ARG | B | 125 | 56.215 | 68.810 | 72.030 | 1.00 | 24.78 |
| ATOM | 6762 | CA | ARG | B | 125 | 57.052 | 68.328 | 73.137 | 1.00 | 25.46 |
| ATOM | 6763 | C | ARG | B | 125 | 56.406 | 68.582 | 74.491 | 1.00 | 24.75 |
| ATOM | 6764 | O | ARG | B | 125 | 56.511 | 67.747 | 75.365 | 1.00 | 26.89 |
| ATOM | 6765 | CB | ARG | B | 125 | 58.429 | 68.947 | 73.085 | 1.00 | 25.96 |
| ATOM | 6766 | CG | ARG | B | 125 | 59.462 | 68.319 | 74.001 | 1.00 | 27.60 |
| ATOM | 6767 | CD | ARG | B | 125 | 60.748 | 69.171 | 74.113 | 1.00 | 28.15 |
| ATOM | 6768 | NE | ARG | B | 125 | 61.355 | 69.340 | 72.796 | 1.00 | 27.56 |
| ATOM | 6769 | CZ | ARG | B | 125 | 62.162 | 68.458 | 72.220 | 1.00 | 28.92 |
| ATOM | 6770 | NH1 | ARG | B | 125 | 62.483 | 67.339 | 72.840 | 1.00 | 30.57 |
| ATOM | 6771 | NH2 | ARG | B | 125 | 62.676 | 68.704 | 71.017 | 1.00 | 29.52 |
| ATOM | 6772 | N | HIS | B | 126 | 55.721 | 69.706 | 74.665 | 1.00 | 24.36 |
| ATOM | 6773 | CA | HIS | B | 126 | 55.100 | 70.043 | 75.958 | 1.00 | 24.50 |
| ATOM | 6774 | C | HIS | B | 126 | 53.593 | 70.130 | 75.879 | 1.00 | 24.67 |
| ATOM | 6775 | O | HIS | B | 126 | 52.905 | 69.909 | 76.857 | 1.00 | 25.37 |
| ATOM | 6776 | CB | HIS | B | 126 | 55.651 | 71.374 | 76.453 | 1.00 | 24.85 |
| ATOM | 6777 | CG | HIS | B | 126 | 57.160 | 71.401 | 76.540 | 1.00 | 25.08 |
| ATOM | 6778 | ND1 | HIS | B | 126 | 57.956 | 72.073 | 75.629 | 1.00 | 27.14 |
| ATOM | 6779 | CD2 | HIS | B | 126 | 58.009 | 70.775 | 77.387 | 1.00 | 23.78 |
| ATOM | 6780 | CE1 | HIS | B | 126 | 59.227 | 71.897 | 75.944 | 1.00 | 24.80 |
| ATOM | 6781 | NE2 | HIS | B | 126 | 59.288 | 71.117 | 77.012 | 1.00 | 25.23 |
| ATOM | 6782 | N | SER | B | 127 | 53.066 | 70.394 | 74.693 | 1.00 | 24.40 |
| ATOM | 6783 | CA | SER | B | 127 | 51.658 | 70.631 | 74.553 | 1.00 | 24.97 |
| ATOM | 6784 | C | SER | B | 127 | 50.885 | 69.340 | 74.516 | 1.00 | 25.44 |
| ATOM | 6785 | O | SER | B | 127 | 51.376 | 68.316 | 74.076 | 1.00 | 25.17 |
| ATOM | 6786 | CB | SER | B | 127 | 51.363 | 71.440 | 73.292 | 1.00 | 24.87 |
| ATOM | 6787 | OG | SER | B | 127 | 52.058 | 70.913 | 72.165 | 1.00 | 22.76 |
| ATOM | 6788 | N | TYR | B | 128 | 49.674 | 69.409 | 75.033 | 1.00 | 27.02 |
| ATOM | 6789 | CA | TYR | B | 128 | 48.758 | 68.291 | 74.979 | 1.00 | 28.11 |
| ATOM | 6790 | C | TYR | B | 128 | 47.368 | 68.716 | 75.343 | 1.00 | 28.88 |
| ATOM | 6791 | O | TYR | B | 128 | 47.150 | 69.827 | 75.838 | 1.00 | 28.25 |
| ATOM | 6792 | CB | TYR | B | 128 | 49.187 | 67.168 | 75.907 | 1.00 | 28.22 |
| ATOM | 6793 | CG | TYR | B | 128 | 49.269 | 67.458 | 77.380 | 1.00 | 30.06 |
| ATOM | 6794 | CD1 | TYR | B | 128 | 48.140 | 67.288 | 78.201 | 1.00 | 31.72 |
| ATOM | 6795 | CD2 | TYR | B | 128 | 50.484 | 67.806 | 77.991 | 1.00 | 29.65 |
| ATOM | 6796 | CE1 | TYR | B | 128 | 48.212 | 67.504 | 79.598 | 1.00 | 31.79 |
| ATOM | 6797 | CE2 | TYR | B | 128 | 50.567 | 68.014 | 79.381 | 1.00 | 29.51 |
| ATOM | 6798 | CZ | TYR | B | 128 | 49.422 | 67.842 | 80.174 | 1.00 | 32.28 |
| ATOM | 6799 | OH | TYR | B | 128 | 49.443 | 68.027 | 81.543 | 1.00 | 34.81 |
| ATOM | 6800 | N | THR | B | 129 | 46.442 | 67.791 | 75.112 | 1.00 | 29.01 |
| ATOM | 6801 | CA | THR | B | 129 | 45.074 | 67.959 | 75.497 | 1.00 | 29.49 |
| ATOM | 6802 | C | THR | B | 129 | 44.674 | 66.844 | 76.495 | 1.00 | 29.76 |
| ATOM | 6803 | O | THR | B | 129 | 45.150 | 65.703 | 76.423 | 1.00 | 29.04 |
| ATOM | 6804 | CB | THR | B | 129 | 44.217 | 67.955 | 74.205 | 1.00 | 29.44 |
| ATOM | 6805 | OG1 | THR | B | 129 | 42.934 | 68.470 | 74.494 | 1.00 | 35.43 |
| ATOM | 6806 | CG2 | THR | B | 129 | 43.907 | 66.606 | 73.732 | 1.00 | 28.93 |
| ATOM | 6807 | N | ALA | B | 130 | 43.812 | 67.195 | 77.440 | 1.00 | 30.39 |
| ATOM | 6808 | CA | ALA | B | 130 | 43.297 | 66.238 | 78.387 | 1.00 | 30.28 |
| ATOM | 6809 | C | ALA | B | 130 | 41.922 | 66.613 | 78.895 | 1.00 | 31.10 |
| ATOM | 6810 | O | ALA | B | 130 | 41.466 | 67.759 | 78.766 | 1.00 | 30.80 |
| ATOM | 6811 | CB | ALA | B | 130 | 44.253 | 66.136 | 79.585 | 1.00 | 30.74 |
| ATOM | 6812 | N | SER | B | 131 | 41.282 | 65.620 | 79.517 | 1.00 | 31.93 |
| ATOM | 6813 | CA | SER | B | 131 | 40.089 | 65.826 | 80.321 | 1.00 | 31.96 |
| ATOM | 6814 | C | SER | B | 131 | 40.512 | 65.949 | 81.790 | 1.00 | 31.93 |
| ATOM | 6815 | O | SER | B | 131 | 41.546 | 65.445 | 82.162 | 1.00 | 32.28 |
| ATOM | 6816 | CB | SER | B | 131 | 39.171 | 64.654 | 80.145 | 1.00 | 31.92 |
| ATOM | 6817 | OG | SER | B | 131 | 38.721 | 64.642 | 78.807 | 1.00 | 34.06 |
| ATOM | 6818 | N | TYR | B | 132 | 39.713 | 66.602 | 82.632 | 1.00 | 31.99 |
| ATOM | 6819 | CA | TYR | B | 132 | 40.099 | 66.805 | 84.018 | 1.00 | 32.05 |
| ATOM | 6820 | C | TYR | B | 132 | 38.923 | 66.642 | 84.977 | 1.00 | 32.91 |
| ATOM | 6821 | O | TYR | B | 132 | 37.862 | 67.210 | 84.772 | 1.00 | 31.84 |
| ATOM | 6822 | CB | TYR | B | 132 | 40.716 | 68.214 | 84.203 | 1.00 | 31.57 |
| ATOM | 6823 | CG | TYR | B | 132 | 42.069 | 68.361 | 83.561 | 1.00 | 32.18 |
| ATOM | 6824 | CD1 | TYR | B | 132 | 43.234 | 67.880 | 84.184 | 1.00 | 32.14 |
| ATOM | 6825 | CD2 | TYR | B | 132 | 42.192 | 68.925 | 82.304 | 1.00 | 31.54 |
| ATOM | 6826 | CE1 | TYR | B | 132 | 44.496 | 67.990 | 83.547 | 1.00 | 33.68 |
| ATOM | 6827 | CE2 | TYR | B | 132 | 43.426 | 69.018 | 81.666 | 1.00 | 33.56 |
| ATOM | 6828 | CZ | TYR | B | 132 | 44.561 | 68.561 | 82.290 | 1.00 | 32.85 |
| ATOM | 6829 | OH | TYR | B | 132 | 45.734 | 68.690 | 81.622 | 1.00 | 34.63 |
| ATOM | 6830 | N | ASP | B | 133 | 39.121 | 65.872 | 86.033 | 1.00 | 35.04 |
| ATOM | 6831 | CA | ASP | B | 133 | 38.138 | 65.818 | 87.102 | 1.00 | 37.05 |
| ATOM | 6832 | C | ASP | B | 133 | 38.750 | 66.493 | 88.306 | 1.00 | 38.09 |
| ATOM | 6833 | O | ASP | B | 133 | 39.942 | 66.770 | 88.326 | 1.00 | 38.74 |
| ATOM | 6834 | CB | ASP | B | 133 | 37.676 | 64.392 | 87.368 | 1.00 | 36.73 |
| ATOM | 6835 | CG | ASP | B | 133 | 36.605 | 63.965 | 86.384 | 1.00 | 38.46 |
| ATOM | 6836 | OD1 | ASP | B | 133 | 35.733 | 64.824 | 86.085 | 1.00 | 38.84 |
| ATOM | 6837 | OD2 | ASP | B | 133 | 36.548 | 62.827 | 85.843 | 1.00 | 38.67 |
| ATOM | 6838 | N | ILE | B | 134 | 37.945 | 66.837 | 89.285 | 1.00 | 39.69 |
| ATOM | 6839 | CA | ILE | B | 134 | 38.499 | 67.440 | 90.495 | 1.00 | 41.09 |
| ATOM | 6840 | C | ILE | B | 134 | 37.970 | 66.675 | 91.691 | 1.00 | 43.16 |
| ATOM | 6841 | O | ILE | B | 134 | 36.779 | 66.539 | 91.822 | 1.00 | 42.96 |
| ATOM | 6842 | CB | ILE | B | 134 | 38.134 | 68.914 | 90.615 | 1.00 | 40.24 |
| ATOM | 6843 | CG1 | ILE | B | 134 | 38.732 | 69.685 | 89.449 | 1.00 | 39.46 |
| ATOM | 6844 | CG2 | ILE | B | 134 | 38.650 | 69.461 | 91.936 | 1.00 | 39.97 |
| ATOM | 6845 | CD1 | ILE | B | 134 | 38.549 | 71.149 | 89.505 | 1.00 | 37.38 |
| ATOM | 6846 | N | TYR | B | 135 | 38.867 | 66.176 | 92.542 | 1.00 | 45.80 |
| ATOM | 6847 | CA | TYR | B | 135 | 38.485 | 65.374 | 93.714 | 1.00 | 48.11 |
| ATOM | 6848 | C | TYR | B | 135 | 38.604 | 66.198 | 94.974 | 1.00 | 49.00 |
| ATOM | 6849 | O | TYR | B | 135 | 39.683 | 66.653 | 95.292 | 1.00 | 48.31 |
| ATOM | 6850 | CB | TYR | B | 135 | 39.408 | 64.156 | 93.819 | 1.00 | 48.71 |
| ATOM | 6851 | CG | TYR | B | 135 | 39.033 | 63.134 | 94.873 | 1.00 | 50.93 |
| ATOM | 6852 | CD1 | TYR | B | 135 | 39.611 | 63.179 | 96.136 | 1.00 | 52.94 |
| ATOM | 6853 | CD2 | TYR | B | 135 | 38.140 | 62.097 | 94.589 | 1.00 | 52.34 |
| ATOM | 6854 | CE1 | TYR | B | 135 | 39.293 | 62.249 | 97.101 | 1.00 | 55.03 |
| ATOM | 6855 | CE2 | TYR | B | 135 | 37.821 | 61.140 | 95.551 | 1.00 | 53.21 |
| ATOM | 6856 | CZ | TYR | B | 135 | 38.403 | 61.227 | 96.808 | 1.00 | 54.72 |
| ATOM | 6857 | OH | TYR | B | 135 | 38.106 | 60.319 | 97.796 | 1.00 | 54.58 |
| ATOM | 6858 | N | ASP | B | 136 | 37.474 | 66.416 | 95.648 | 1.00 | 51.11 |
| ATOM | 6859 | CA | ASP | B | 136 | 37.415 | 67.122 | 96.932 | 1.00 | 52.49 |
| ATOM | 6860 | C | ASP | B | 136 | 37.934 | 66.142 | 97.945 | 1.00 | 53.83 |
| ATOM | 6861 | O | ASP | B | 136 | 37.364 | 65.080 | 98.100 | 1.00 | 53.57 |
| ATOM | 6862 | CB | ASP | B | 136 | 35.962 | 67.442 | 97.290 | 1.00 | 52.71 |
| ATOM | 6863 | CG | ASP | B | 136 | 35.830 | 68.308 | 98.530 | 1.00 | 51.80 |
| ATOM | 6864 | OD1 | ASP | B | 136 | 36.742 | 68.293 | 99.384 | 1.00 | 49.25 |
| ATOM | 6865 | OD2 | ASP | B | 136 | 34.829 | 69.044 | 98.718 | 1.00 | 51.13 |
| ATOM | 6866 | N | LEU | B | 137 | 39.018 | 66.483 | 98.623 | 1.00 | 55.57 |
| ATOM | 6867 | CA | LEU | B | 137 | 39.616 | 65.553 | 99.557 | 1.00 | 57.54 |
| ATOM | 6868 | C | LEU | B | 137 | 38.884 | 65.516 | 100.917 | 1.00 | 59.20 |
| ATOM | 6869 | O | LEU | B | 137 | 38.734 | 64.447 | 101.491 | 1.00 | 59.25 |
| ATOM | 6870 | CB | LEU | B | 137 | 41.112 | 65.852 | 99.718 | 1.00 | 57.29 |
| ATOM | 6871 | CG | LEU | B | 137 | 41.972 | 65.236 | 98.612 | 1.00 | 58.13 |
| ATOM | 6872 | CD1 | LEU | B | 137 | 43.199 | 66.071 | 98.305 | 1.00 | 58.23 |
| ATOM | 6873 | CD2 | LEU | B | 137 | 42.363 | 63.813 | 98.978 | 1.00 | 58.48 |
| ATOM | 6874 | N | ASN | B | 138 | 38.422 | 66.666 | 101.415 | 1.00 | 60.98 |
| ATOM | 6875 | CA | ASN | B | 138 | 37.721 | 66.714 | 102.705 | 1.00 | 62.32 |
| ATOM | 6876 | C | ASN | B | 138 | 36.383 | 65.993 | 102.627 | 1.00 | 63.17 |
| ATOM | 6877 | O | ASN | B | 138 | 35.557 | 66.058 | 103.543 | 1.00 | 63.85 |
| ATOM | 6878 | CB | ASN | B | 138 | 37.535 | 68.169 | 103.185 | 1.00 | 62.59 |
| ATOM | 6879 | CG | ASN | B | 138 | 38.691 | 68.649 | 104.046 | 1.00 | 63.40 |
| ATOM | 6880 | OD1 | ASN | B | 138 | 38.575 | 68.746 | 105.268 | 1.00 | 64.31 |
| ATOM | 6881 | ND2 | ASN | B | 138 | 39.821 | 68.930 | 103.411 | 1.00 | 63.58 |
| ATOM | 6882 | N | LYS | B | 139 | 36.181 | 65.325 | 101.499 | 1.00 | 64.15 |
| ATOM | 6883 | CA | LYS | B | 139 | 35.023 | 64.493 | 101.266 | 1.00 | 64.69 |
| ATOM | 6884 | C | LYS | B | 139 | 35.532 | 63.413 | 100.320 | 1.00 | 65.21 |
| ATOM | 6885 | O | LYS | B | 139 | 36.479 | 63.642 | 99.585 | 1.00 | 65.06 |
| ATOM | 6886 | CB | LYS | B | 139 | 33.922 | 65.299 | 100.590 | 1.00 | 64.96 |
| ATOM | 6887 | CG | LYS | B | 139 | 33.728 | 66.744 | 101.069 | 1.00 | 64.79 |
| ATOM | 6888 | CD | LYS | B | 139 | 32.471 | 67.315 | 100.391 | 1.00 | 65.33 |
| ATOM | 6889 | CE | LYS | B | 139 | 32.153 | 68.768 | 100.757 | 1.00 | 65.49 |
| ATOM | 6890 | NZ | LYS | B | 139 | 31.094 | 69.330 | 99.832 | 1.00 | 63.71 |
| ATOM | 6891 | N | ARG | B | 140 | 34.956 | 62.222 | 100.335 | 1.00 | 65.77 |
| ATOM | 6892 | CA | ARG | B | 140 | 35.388 | 61.205 | 99.367 | 1.00 | 66.20 |
| ATOM | 6893 | C | ARG | B | 140 | 34.682 | 61.468 | 98.038 | 1.00 | 64.94 |
| ATOM | 6894 | O | ARG | B | 140 | 34.056 | 60.551 | 97.493 | 1.00 | 65.35 |
| ATOM | 6895 | CB | ARG | B | 140 | 34.966 | 59.796 | 99.807 | 1.00 | 67.02 |
| ATOM | 6896 | CG | ARG | B | 140 | 35.718 | 59.136 | 100.971 | 1.00 | 70.61 |
| ATOM | 6897 | CD | ARG | B | 140 | 35.212 | 57.700 | 101.215 | 1.00 | 74.78 |
| ATOM | 6898 | NE | ARG | B | 140 | 35.526 | 57.150 | 102.535 | 1.00 | 78.56 |
| ATOM | 6899 | CZ | ARG | B | 140 | 36.668 | 56.555 | 102.863 | 1.00 | 81.17 |
| ATOM | 6900 | NH1 | ARG | B | 140 | 37.656 | 56.438 | 101.977 | 1.00 | 82.23 |
| ATOM | 6901 | NH2 | ARG | B | 140 | 36.828 | 56.082 | 104.094 | 1.00 | 81.57 |
| ATOM | 6902 | N | GLN | B | 141 | 34.756 | 62.688 | 97.508 | 1.00 | 63.09 |
| ATOM | 6903 | CA | GLN | B | 141 | 33.921 | 63.024 | 96.353 | 1.00 | 61.89 |
| ATOM | 6904 | C | GLN | B | 141 | 34.563 | 63.756 | 95.163 | 1.00 | 59.88 |
| ATOM | 6905 | O | GLN | B | 141 | 35.290 | 64.735 | 95.317 | 1.00 | 58.49 |
| ATOM | 6906 | CB | GLN | B | 141 | 32.731 | 63.889 | 96.821 | 1.00 | 62.00 |
| ATOM | 6907 | CG | GLN | B | 141 | 31.581 | 63.186 | 97.546 | 1.00 | 63.58 |
| ATOM | 6908 | CD | GLN | B | 141 | 30.365 | 64.125 | 97.737 | 1.00 | 65.48 |
| ATOM | 6909 | OE1 | GLN | B | 141 | 30.518 | 65.355 | 97.718 | 1.00 | 66.14 |
| ATOM | 6910 | NE2 | GLN | B | 141 | 29.172 | 63.548 | 97.909 | 1.00 | 64.71 |
| ATOM | 6911 | N | LEU | B | 142 | 34.226 | 63.276 | 93.970 | 1.00 | 58.41 |
| ATOM | 6912 | CA | LEU | B | 142 | 34.549 | 63.956 | 92.724 | 1.00 | 57.40 |
| ATOM | 6913 | C | LEU | B | 142 | 33.516 | 65.075 | 92.566 | 1.00 | 56.15 |
| ATOM | 6914 | O | LEU | B | 142 | 32.351 | 64.887 | 92.903 | 1.00 | 55.20 |
| ATOM | 6915 | CB | LEU | B | 142 | 34.439 | 62.983 | 91.546 | 1.00 | 57.42 |
| ATOM | 6916 | CG | LEU | B | 142 | 35.432 | 61.806 | 91.423 | 1.00 | 58.38 |
| ATOM | 6917 | CD1 | LEU | B | 142 | 34.881 | 60.708 | 90.505 | 1.00 | 58.70 |
| ATOM | 6918 | CD2 | LEU | B | 142 | 36.823 | 62.241 | 90.928 | 1.00 | 57.90 |
| ATOM | 6919 | N | ILE | B | 143 | 33.910 | 66.251 | 92.096 | 1.00 | 54.79 |
| ATOM | 6920 | CA | ILE | B | 143 | 32.892 | 67.270 | 91.892 | 1.00 | 54.52 |
| ATOM | 6921 | C | ILE | B | 143 | 32.236 | 66.971 | 90.554 | 1.00 | 53.58 |
| ATOM | 6922 | O | ILE | B | 143 | 32.897 | 66.682 | 89.568 | 1.00 | 53.27 |
| ATOM | 6923 | CB | ILE | B | 143 | 33.420 | 68.714 | 91.929 | 1.00 | 54.74 |
| ATOM | 6924 | CG1 | ILE | B | 143 | 33.846 | 69.140 | 90.543 | 1.00 | 55.65 |
| ATOM | 6925 | CG2 | ILE | B | 143 | 34.528 | 68.876 | 92.987 | 1.00 | 54.99 |
| ATOM | 6926 | CD1 | ILE | B | 143 | 33.652 | 70.604 | 90.273 | 1.00 | 56.96 |
| ATOM | 6927 | N | THR | B | 144 | 30.922 | 67.044 | 90.547 | 1.00 | 52.14 |
| ATOM | 6928 | CA | THR | B | 144 | 30.130 | 66.662 | 89.410 | 1.00 | 51.43 |
| ATOM | 6929 | C | THR | B | 144 | 29.588 | 67.855 | 88.608 | 1.00 | 49.50 |
| ATOM | 6930 | O | THR | B | 144 | 29.032 | 67.663 | 87.546 | 1.00 | 49.43 |
| ATOM | 6931 | CB | THR | B | 144 | 28.989 | 65.746 | 89.966 | 1.00 | 51.93 |
| ATOM | 6932 | OG1 | THR | B | 144 | 29.462 | 64.382 | 90.017 | 1.00 | 52.78 |
| ATOM | 6933 | CG2 | THR | B | 144 | 27.758 | 65.689 | 89.053 | 1.00 | 53.08 |
| ATOM | 6934 | N | GLU | B | 145 | 29.791 | 69.075 | 89.099 | 1.00 | 47.72 |
| ATOM | 6935 | CA | GLU | B | 145 | 29.212 | 70.284 | 88.490 | 1.00 | 46.86 |
| ATOM | 6936 | C | GLU | B | 145 | 30.290 | 71.195 | 87.943 | 1.00 | 44.59 |
| ATOM | 6937 | O | GLU | B | 145 | 31.366 | 71.145 | 88.421 | 1.00 | 42.44 |
| ATOM | 6938 | CB | GLU | B | 145 | 28.497 | 71.160 | 89.547 | 1.00 | 46.71 |
| ATOM | 6939 | CG | GLU | B | 145 | 27.623 | 70.473 | 90.570 | 1.00 | 48.46 |
| ATOM | 6940 | CD | GLU | B | 145 | 26.846 | 71.497 | 91.401 | 1.00 | 50.31 |
| ATOM | 6941 | OE1 | GLU | B | 145 | 26.968 | 71.449 | 92.665 | 1.00 | 52.18 |
| ATOM | 6942 | OE2 | GLU | B | 145 | 26.156 | 72.368 | 90.784 | 1.00 | 46.09 |
| ATOM | 6943 | N | GLU | B | 146 | 29.958 | 72.075 | 86.998 | 1.00 | 43.57 |
| ATOM | 6944 | CA | GLU | B | 146 | 30.931 | 73.056 | 86.481 | 1.00 | 42.81 |
| ATOM | 6945 | C | GLU | B | 146 | 32.323 | 72.410 | 86.287 | 1.00 | 40.46 |
| ATOM | 6946 | O | GLU | B | 146 | 33.315 | 72.856 | 86.852 | 1.00 | 39.08 |
| ATOM | 6947 | CB | GLU | B | 146 | 31.052 | 74.245 | 87.454 | 1.00 | 43.05 |
| ATOM | 6948 | CG | GLU | B | 146 | 29.723 | 74.917 | 87.818 | 1.00 | 45.72 |
| ATOM | 6949 | CD | GLU | B | 146 | 28.904 | 75.360 | 86.627 | 1.00 | 46.80 |
| ATOM | 6950 | OE1 | GLU | B | 146 | 29.471 | 75.547 | 85.539 | 1.00 | 47.90 |
| ATOM | 6951 | OE2 | GLU | B | 146 | 27.679 | 75.521 | 86.775 | 1.00 | 48.73 |
| ATOM | 6952 | N | ARG | B | 147 | 32.359 | 71.331 | 85.531 | 1.00 | 38.23 |
| ATOM | 6953 | CA | ARG | B | 147 | 33.580 | 70.611 | 85.318 | 1.00 | 37.89 |
| ATOM | 6954 | C | ARG | B | 147 | 34.416 | 71.336 | 84.280 | 1.00 | 35.94 |
| ATOM | 6955 | O | ARG | B | 147 | 33.909 | 71.963 | 83.342 | 1.00 | 36.10 |
| ATOM | 6956 | CB | ARG | B | 147 | 33.302 | 69.182 | 84.821 | 1.00 | 38.26 |
| ATOM | 6957 | CG | ARG | B | 147 | 32.877 | 68.097 | 85.866 | 1.00 | 40.91 |
| ATOM | 6958 | CD | ARG | B | 147 | 32.619 | 66.719 | 85.170 | 1.00 | 46.23 |
| ATOM | 6959 | NE | ARG | B | 147 | 31.968 | 65.683 | 85.989 | 1.00 | 50.43 |
| ATOM | 6960 | CZ | ARG | B | 147 | 32.447 | 64.445 | 86.217 | 1.00 | 53.41 |
| ATOM | 6961 | NH1 | ARG | B | 147 | 33.618 | 64.053 | 85.735 | 1.00 | 53.39 |
| ATOM | 6962 | NH2 | ARG | B | 147 | 31.756 | 63.590 | 86.967 | 1.00 | 55.20 |
| ATOM | 6963 | N | ILE | B | 148 | 35.717 | 71.220 | 84.456 | 1.00 | 33.99 |
| ATOM | 6964 | CA | ILE | B | 148 | 36.693 | 71.657 | 83.471 | 1.00 | 31.85 |
| ATOM | 6965 | C | ILE | B | 148 | 36.340 | 70.859 | 82.218 | 1.00 | 30.03 |
| ATOM | 6966 | O | ILE | B | 148 | 36.159 | 69.669 | 82.298 | 1.00 | 28.67 |
| ATOM | 6967 | CB | ILE | B | 148 | 38.094 | 71.286 | 84.013 | 1.00 | 31.92 |
| ATOM | 6968 | CG1 | ILE | B | 148 | 38.473 | 72.268 | 85.147 | 1.00 | 34.33 |
| ATOM | 6969 | CG2 | ILE | B | 148 | 39.125 | 71.286 | 82.935 | 1.00 | 30.99 |
| ATOM | 6970 | CD1 | ILE | B | 148 | 39.951 | 72.101 | 85.737 | 1.00 | 34.47 |
| ATOM | 6971 | N | PRO | B | 149 | 36.240 | 71.484 | 81.062 | 1.00 | 28.84 |
| ATOM | 6972 | CA | PRO | B | 149 | 35.799 | 70.742 | 79.884 | 1.00 | 28.65 |
| ATOM | 6973 | C | PRO | B | 149 | 36.804 | 69.738 | 79.409 | 1.00 | 28.89 |
| ATOM | 6974 | O | PRO | B | 149 | 37.980 | 69.796 | 79.776 | 1.00 | 28.38 |
| ATOM | 6975 | CB | PRO | B | 149 | 35.601 | 71.820 | 78.802 | 1.00 | 28.36 |
| ATOM | 6976 | CG | PRO | B | 149 | 36.146 | 73.067 | 79.323 | 1.00 | 28.39 |
| ATOM | 6977 | CD | PRO | B | 149 | 36.544 | 72.892 | 80.760 | 1.00 | 29.09 |
| ATOM | 6978 | N | ASN | B | 150 | 36.318 | 68.799 | 78.614 | 1.00 | 29.51 |
| ATOM | 6979 | CA | ASN | B | 150 | 37.173 | 67.878 | 77.874 | 1.00 | 30.82 |
| ATOM | 6980 | C | ASN | B | 150 | 38.036 | 68.676 | 76.857 | 1.00 | 30.13 |
| ATOM | 6981 | O | ASN | B | 150 | 37.696 | 69.805 | 76.516 | 1.00 | 29.44 |
| ATOM | 6982 | CB | ASN | B | 150 | 36.281 | 66.855 | 77.146 | 1.00 | 31.55 |
| ATOM | 6983 | CG | ASN | B | 150 | 35.515 | 65.954 | 78.114 | 1.00 | 37.05 |
| ATOM | 6984 | OD1 | ASN | B | 150 | 35.879 | 65.831 | 79.298 | 1.00 | 36.83 |
| ATOM | 6985 | ND2 | ASN | B | 150 | 34.467 | 65.304 | 77.616 | 1.00 | 45.68 |
| ATOM | 6986 | N | ASN | B | 151 | 39.132 | 68.089 | 76.368 | 1.00 | 30.36 |
| ATOM | 6987 | CA | ASN | B | 151 | 40.029 | 68.784 | 75.425 | 1.00 | 30.63 |
| ATOM | 6988 | C | ASN | B | 151 | 40.568 | 70.080 | 76.006 | 1.00 | 29.87 |
| ATOM | 6989 | O | ASN | B | 151 | 40.780 | 71.032 | 75.268 | 1.00 | 30.45 |
| ATOM | 6990 | CB | ASN | B | 151 | 39.323 | 69.140 | 74.095 | 1.00 | 31.24 |
| ATOM | 6991 | CG | ASN | B | 151 | 38.597 | 67.942 | 73.466 | 1.00 | 33.32 |
| ATOM | 6992 | OD1 | ASN | B | 151 | 39.228 | 66.989 | 73.033 | 1.00 | 38.87 |
| ATOM | 6993 | ND2 | ASN | B | 151 | 37.277 | 67.986 | 73.435 | 1.00 | 33.94 |
| ATOM | 6994 | N | THR | B | 152 | 40.734 | 70.156 | 77.323 | 1.00 | 28.47 |
| ATOM | 6995 | CA | THR | B | 152 | 41.344 | 71.333 | 77.906 | 1.00 | 27.81 |
| ATOM | 6996 | C | THR | B | 152 | 42.811 | 71.275 | 77.522 | 1.00 | 27.45 |
| ATOM | 6997 | O | THR | B | 152 | 43.376 | 70.198 | 77.451 | 1.00 | 27.18 |
| ATOM | 6998 | CB | THR | B | 152 | 41.106 | 71.365 | 79.391 | 1.00 | 27.63 |
| ATOM | 6999 | OG1 | THR | B | 152 | 39.765 | 71.824 | 79.629 | 1.00 | 30.57 |
| ATOM | 7000 | CG2 | THR | B | 152 | 41.952 | 72.360 | 80.087 | 1.00 | 27.37 |
| ATOM | 7001 | N | GLN | B | 153 | 43.381 | 72.439 | 77.232 | 1.00 | 27.50 |
| ATOM | 7002 | CA | GLN | B | 153 | 44.743 | 72.586 | 76.699 | 1.00 | 27.91 |
| ATOM | 7003 | C | GLN | B | 153 | 45.821 | 72.836 | 77.764 | 1.00 | 28.03 |
| ATOM | 7004 | O | GLN | B | 153 | 46.979 | 72.527 | 77.573 | 1.00 | 27.08 |
| ATOM | 7005 | CB | GLN | B | 153 | 44.750 | 73.743 | 75.686 | 1.00 | 27.94 |
| ATOM | 7006 | CG | GLN | B | 153 | 44.107 | 73.436 | 74.316 | 1.00 | 26.46 |
| ATOM | 7007 | CD | GLN | B | 153 | 43.694 | 74.713 | 73.587 | 1.00 | 27.80 |
| ATOM | 7008 | OE1 | GLN | B | 153 | 42.905 | 75.503 | 74.125 | 1.00 | 28.54 |
| ATOM | 7009 | NE2 | GLN | B | 153 | 44.242 | 74.941 | 72.398 | 1.00 | 25.93 |
| ATOM | 7010 | N | TRP | B | 154 | 45.430 | 73.462 | 78.862 | 1.00 | 28.53 |
| ATOM | 7011 | CA | TRP | B | 154 | 46.316 | 73.658 | 79.961 | 1.00 | 28.10 |
| ATOM | 7012 | C | TRP | B | 154 | 45.476 | 73.969 | 81.180 | 1.00 | 28.08 |
| ATOM | 7013 | O | TRP | B | 154 | 44.400 | 74.514 | 81.035 | 1.00 | 26.97 |
| ATOM | 7014 | CB | TRP | B | 154 | 47.285 | 74.803 | 79.670 | 1.00 | 29.09 |
| ATOM | 7015 | CG | TRP | B | 154 | 48.174 | 75.032 | 80.832 | 1.00 | 30.42 |
| ATOM | 7016 | CD1 | TRP | B | 154 | 48.067 | 76.007 | 81.758 | 1.00 | 34.21 |
| ATOM | 7017 | CD2 | TRP | B | 154 | 49.260 | 74.221 | 81.218 | 1.00 | 29.80 |
| ATOM | 7018 | NE1 | TRP | B | 154 | 49.043 | 75.865 | 82.711 | 1.00 | 34.81 |
| ATOM | 7019 | CE2 | TRP | B | 154 | 49.794 | 74.765 | 82.399 | 1.00 | 33.62 |
| ATOM | 7020 | CE3 | TRP | B | 154 | 49.849 | 73.080 | 80.677 | 1.00 | 31.67 |
| ATOM | 7021 | CZ2 | TRP | B | 154 | 50.901 | 74.211 | 83.065 | 1.00 | 33.57 |
| ATOM | 7022 | CZ3 | TRP | B | 154 | 50.963 | 72.530 | 81.329 | 1.00 | 33.77 |
| ATOM | 7023 | CH2 | TRP | B | 154 | 51.468 | 73.100 | 82.511 | 1.00 | 35.86 |
| ATOM | 7024 | N | VAL | B | 155 | 45.944 | 73.584 | 82.372 | 1.00 | 27.30 |
| ATOM | 7025 | CA | VAL | B | 155 | 45.295 | 73.925 | 83.625 | 1.00 | 28.02 |
| ATOM | 7026 | C | VAL | B | 155 | 46.323 | 74.186 | 84.721 | 1.00 | 28.50 |
| ATOM | 7027 | O | VAL | B | 155 | 47.293 | 73.499 | 84.825 | 1.00 | 27.25 |
| ATOM | 7028 | CB | VAL | B | 155 | 44.468 | 72.766 | 84.254 | 1.00 | 28.49 |
| ATOM | 7029 | CG1 | VAL | B | 155 | 43.605 | 73.290 | 85.381 | 1.00 | 27.80 |
| ATOM | 7030 | CG2 | VAL | B | 155 | 43.669 | 72.012 | 83.259 | 1.00 | 29.00 |
| ATOM | 7031 | N | THR | B | 156 | 46.050 | 75.109 | 85.605 | 1.00 | 29.64 |
| ATOM | 7032 | CA | THR | B | 156 | 46.963 | 75.351 | 86.704 | 1.00 | 31.54 |
| ATOM | 7033 | C | THR | B | 156 | 46.229 | 75.874 | 87.899 | 1.00 | 31.50 |
| ATOM | 7034 | O | THR | B | 156 | 45.432 | 76.827 | 87.774 | 1.00 | 31.11 |
| ATOM | 7035 | CB | THR | B | 156 | 47.971 | 76.511 | 86.433 | 1.00 | 31.07 |
| ATOM | 7036 | OG1 | THR | B | 156 | 48.561 | 76.429 | 85.136 | 1.00 | 37.41 |
| ATOM | 7037 | CG2 | THR | B | 156 | 49.096 | 76.360 | 87.335 | 1.00 | 31.91 |
| ATOM | 7038 | N | TRP | B | 157 | 46.614 | 75.350 | 89.058 | 1.00 | 32.16 |
| ATOM | 7039 | CA | TRP | B | 157 | 46.212 | 75.919 | 90.344 | 1.00 | 32.69 |
| ATOM | 7040 | C | TRP | B | 157 | 46.976 | 77.210 | 90.519 | 1.00 | 33.37 |
| ATOM | 7041 | O | TRP | B | 157 | 48.056 | 77.340 | 89.997 | 1.00 | 33.68 |
| ATOM | 7042 | CB | TRP | B | 157 | 46.644 | 74.988 | 91.509 | 1.00 | 32.93 |
| ATOM | 7043 | CG | TRP | B | 157 | 45.962 | 73.635 | 91.559 | 1.00 | 30.74 |
| ATOM | 7044 | CD1 | TRP | B | 157 | 46.539 | 72.441 | 91.353 | 1.00 | 28.69 |
| ATOM | 7045 | CD2 | TRP | B | 157 | 44.597 | 73.371 | 91.906 | 1.00 | 28.30 |
| ATOM | 7046 | NE1 | TRP | B | 157 | 45.618 | 71.434 | 91.530 | 1.00 | 31.58 |
| ATOM | 7047 | CE2 | TRP | B | 157 | 44.412 | 71.993 | 91.857 | 1.00 | 29.40 |
| ATOM | 7048 | CE3 | TRP | B | 157 | 43.497 | 74.180 | 92.225 | 1.00 | 34.40 |
| ATOM | 7049 | CZ2 | TRP | B | 157 | 43.171 | 71.385 | 92.105 | 1.00 | 34.85 |
| ATOM | 7050 | CZ3 | TRP | B | 157 | 42.268 | 73.581 | 92.485 | 1.00 | 34.99 |
| ATOM | 7051 | CH2 | TRP | B | 157 | 42.117 | 72.194 | 92.423 | 1.00 | 35.01 |
| ATOM | 7052 | N | SER | B | 158 | 46.419 | 78.156 | 91.268 | 1.00 | 34.79 |
| ATOM | 7053 | CA | SER | B | 158 | 47.129 | 79.373 | 91.686 | 1.00 | 34.84 |
| ATOM | 7054 | C | SER | B | 158 | 48.159 | 78.893 | 92.697 | 1.00 | 34.81 |
| ATOM | 7055 | O | SER | B | 158 | 48.094 | 77.783 | 93.110 | 1.00 | 34.12 |
| ATOM | 7056 | CB | SER | B | 158 | 46.148 | 80.317 | 92.351 | 1.00 | 35.28 |
| ATOM | 7057 | OG | SER | B | 158 | 45.236 | 79.558 | 93.138 | 1.00 | 35.00 |
| ATOM | 7058 | N | PRO | B | 159 | 49.135 | 79.690 | 93.062 | 1.00 | 35.67 |
| ATOM | 7059 | CA | PRO | B | 159 | 50.207 | 79.190 | 93.919 | 1.00 | 36.90 |
| ATOM | 7060 | C | PRO | B | 159 | 49.799 | 78.909 | 95.352 | 1.00 | 38.17 |
| ATOM | 7061 | O | PRO | B | 159 | 50.396 | 78.066 | 96.000 | 1.00 | 39.07 |
| ATOM | 7062 | CB | PRO | B | 159 | 51.263 | 80.288 | 93.848 | 1.00 | 36.48 |
| ATOM | 7063 | CG | PRO | B | 159 | 50.882 | 81.140 | 92.744 | 1.00 | 36.04 |
| ATOM | 7064 | CD | PRO | B | 159 | 49.373 | 81.072 | 92.635 | 1.00 | 36.34 |
| ATOM | 7065 | N | VAL | B | 160 | 48.787 | 79.601 | 95.844 | 1.00 | 39.52 |
| ATOM | 7066 | CA | VAL | B | 160 | 48.314 | 79.362 | 97.192 | 1.00 | 39.85 |
| ATOM | 7067 | C | VAL | B | 160 | 46.849 | 79.215 | 96.995 | 1.00 | 39.62 |
| ATOM | 7068 | O | VAL | B | 160 | 46.318 | 79.748 | 96.043 | 1.00 | 40.31 |
| ATOM | 7069 | CB | VAL | B | 160 | 48.616 | 80.549 | 98.116 | 1.00 | 40.71 |
| ATOM | 7070 | CG1 | VAL | B | 160 | 50.140 | 80.769 | 98.235 | 1.00 | 41.63 |
| ATOM | 7071 | CG2 | VAL | B | 160 | 47.946 | 81.801 | 97.605 | 1.00 | 41.78 |
| ATOM | 7072 | N | GLY | B | 161 | 46.194 | 78.435 | 97.832 | 1.00 | 38.75 |
| ATOM | 7073 | CA | GLY | B | 161 | 44.758 | 78.307 | 97.765 | 1.00 | 38.17 |
| ATOM | 7074 | C | GLY | B | 161 | 44.285 | 77.225 | 96.830 | 1.00 | 37.54 |
| ATOM | 7075 | O | GLY | B | 161 | 44.794 | 76.100 | 96.821 | 1.00 | 36.68 |
| ATOM | 7076 | N | HIS | B | 162 | 43.243 | 77.543 | 96.076 | 1.00 | 36.80 |
| ATOM | 7077 | CA | HIS | B | 162 | 42.734 | 76.559 | 95.160 | 1.00 | 36.56 |
| ATOM | 7078 | C | HIS | B | 162 | 41.957 | 77.155 | 93.993 | 1.00 | 34.81 |
| ATOM | 7079 | O | HIS | B | 162 | 41.067 | 76.508 | 93.466 | 1.00 | 34.79 |
| ATOM | 7080 | CB | HIS | B | 162 | 41.902 | 75.554 | 95.938 | 1.00 | 37.14 |
| ATOM | 7081 | CG | HIS | B | 162 | 40.770 | 76.177 | 96.682 | 1.00 | 40.25 |
| ATOM | 7082 | ND1 | HIS | B | 162 | 40.455 | 75.837 | 97.977 | 1.00 | 42.53 |
| ATOM | 7083 | CD2 | HIS | B | 162 | 39.872 | 77.118 | 96.309 | 1.00 | 42.08 |
| ATOM | 7084 | CE1 | HIS | B | 162 | 39.415 | 76.548 | 98.376 | 1.00 | 43.68 |
| ATOM | 7085 | NE2 | HIS | B | 162 | 39.042 | 77.332 | 97.382 | 1.00 | 44.43 |
| ATOM | 7086 | N | LYS | B | 163 | 42.272 | 78.387 | 93.604 | 1.00 | 33.74 |
| ATOM | 7087 | CA | LYS | B | 163 | 41.727 | 78.953 | 92.370 | 1.00 | 34.09 |
| ATOM | 7088 | C | LYS | B | 163 | 42.340 | 78.168 | 91.224 | 1.00 | 32.87 |
| ATOM | 7089 | O | LYS | B | 163 | 43.407 | 77.657 | 91.394 | 1.00 | 32.77 |
| ATOM | 7090 | CB | LYS | B | 163 | 42.114 | 80.411 | 92.173 | 1.00 | 34.03 |
| ATOM | 7091 | CG | LYS | B | 163 | 41.512 | 81.400 | 93.157 | 1.00 | 34.96 |
| ATOM | 7092 | CD | LYS | B | 163 | 42.055 | 82.788 | 92.797 | 1.00 | 34.44 |
| ATOM | 7093 | CE | LYS | B | 163 | 41.737 | 83.843 | 93.846 | 1.00 | 34.13 |
| ATOM | 7094 | NZ | LYS | B | 163 | 42.162 | 85.145 | 93.358 | 1.00 | 30.81 |
| ATOM | 7095 | N | LEU | B | 164 | 41.635 | 78.028 | 90.107 | 1.00 | 32.21 |
| ATOM | 7096 | CA | LEU | B | 164 | 42.143 | 77.331 | 88.918 | 1.00 | 32.26 |
| ATOM | 7097 | C | LEU | B | 164 | 42.058 | 78.224 | 87.703 | 1.00 | 30.77 |
| ATOM | 7098 | O | LEU | B | 164 | 41.179 | 79.059 | 87.588 | 1.00 | 30.09 |
| ATOM | 7099 | CB | LEU | B | 164 | 41.308 | 76.107 | 88.570 | 1.00 | 32.35 |
| ATOM | 7100 | CG | LEU | B | 164 | 41.380 | 74.840 | 89.397 | 1.00 | 35.85 |
| ATOM | 7101 | CD1 | LEU | B | 164 | 40.073 | 74.031 | 89.319 | 1.00 | 37.85 |
| ATOM | 7102 | CD2 | LEU | B | 164 | 42.499 | 73.968 | 88.980 | 1.00 | 37.12 |
| ATOM | 7103 | N | ALA | B | 165 | 42.982 | 78.018 | 86.789 | 1.00 | 29.97 |
| ATOM | 7104 | CA | ALA | B | 165 | 42.969 | 78.719 | 85.527 | 1.00 | 29.70 |
| ATOM | 7105 | C | ALA | B | 165 | 43.264 | 77.683 | 84.452 | 1.00 | 29.05 |
| ATOM | 7106 | O | ALA | B | 165 | 44.157 | 76.845 | 84.621 | 1.00 | 29.22 |
| ATOM | 7107 | CB | ALA | B | 165 | 43.985 | 79.805 | 85.522 | 1.00 | 29.61 |
| ATOM | 7108 | N | TYR | B | 166 | 42.503 | 77.720 | 83.371 | 1.00 | 27.94 |
| ATOM | 7109 | CA | TYR | B | 166 | 42.702 | 76.775 | 82.286 | 1.00 | 27.80 |
| ATOM | 7110 | C | TYR | B | 166 | 42.479 | 77.394 | 80.911 | 1.00 | 27.07 |
| ATOM | 7111 | O | TYR | B | 166 | 41.950 | 78.492 | 80.773 | 1.00 | 26.60 |
| ATOM | 7112 | CB | TYR | B | 166 | 41.811 | 75.548 | 82.478 | 1.00 | 28.31 |
| ATOM | 7113 | CG | TYR | B | 166 | 40.313 | 75.795 | 82.488 | 1.00 | 28.99 |
| ATOM | 7114 | CD1 | TYR | B | 166 | 39.598 | 75.876 | 81.296 | 1.00 | 31.31 |
| ATOM | 7115 | CD2 | TYR | B | 166 | 39.607 | 75.893 | 83.680 | 1.00 | 30.82 |
| ATOM | 7116 | CE1 | TYR | B | 166 | 38.228 | 76.073 | 81.273 | 1.00 | 31.44 |
| ATOM | 7117 | CE2 | TYR | B | 166 | 38.204 | 76.077 | 83.669 | 1.00 | 33.40 |
| ATOM | 7118 | CZ | TYR | B | 166 | 37.528 | 76.179 | 82.450 | 1.00 | 31.55 |
| ATOM | 7119 | OH | TYR | B | 166 | 36.150 | 76.348 | 82.390 | 1.00 | 30.35 |
| ATOM | 7120 | N | VAL | B | 167 | 42.945 | 76.692 | 79.901 | 1.00 | 26.39 |
| ATOM | 7121 | CA | VAL | B | 167 | 42.799 | 77.133 | 78.540 | 1.00 | 26.34 |
| ATOM | 7122 | C | VAL | B | 167 | 42.038 | 76.045 | 77.759 | 1.00 | 25.99 |
| ATOM | 7123 | O | VAL | B | 167 | 42.388 | 74.866 | 77.788 | 1.00 | 25.38 |
| ATOM | 7124 | CB | VAL | B | 167 | 44.171 | 77.442 | 77.908 | 1.00 | 26.71 |
| ATOM | 7125 | CG1 | VAL | B | 167 | 44.041 | 77.790 | 76.447 | 1.00 | 27.08 |
| ATOM | 7126 | CG2 | VAL | B | 167 | 44.858 | 78.584 | 78.671 | 1.00 | 26.78 |
| ATOM | 7127 | N | TRP | B | 168 | 40.993 | 76.481 | 77.073 | 1.00 | 25.62 |
| ATOM | 7128 | CA | TRP | B | 168 | 40.125 | 75.616 | 76.311 | 1.00 | 26.65 |
| ATOM | 7129 | C | TRP | B | 168 | 39.689 | 76.403 | 75.089 | 1.00 | 26.23 |
| ATOM | 7130 | O | TRP | B | 168 | 39.330 | 77.566 | 75.176 | 1.00 | 25.50 |
| ATOM | 7131 | CB | TRP | B | 168 | 38.953 | 75.181 | 77.160 | 1.00 | 26.65 |
| ATOM | 7132 | CG | TRP | B | 168 | 37.956 | 74.299 | 76.417 | 1.00 | 28.92 |
| ATOM | 7133 | CD1 | TRP | B | 168 | 37.991 | 72.940 | 76.290 | 1.00 | 28.41 |
| ATOM | 7134 | CD2 | TRP | B | 168 | 36.758 | 74.723 | 75.782 | 1.00 | 29.22 |
| ATOM | 7135 | NE1 | TRP | B | 168 | 36.886 | 72.499 | 75.599 | 1.00 | 29.45 |
| ATOM | 7136 | CE2 | TRP | B | 168 | 36.106 | 73.570 | 75.287 | 1.00 | 28.79 |
| ATOM | 7137 | CE3 | TRP | B | 168 | 36.154 | 75.962 | 75.588 | 1.00 | 31.73 |
| ATOM | 7138 | CZ2 | TRP | B | 168 | 34.909 | 73.622 | 74.602 | 1.00 | 30.76 |
| ATOM | 7139 | CZ3 | TRP | B | 168 | 34.949 | 76.013 | 74.909 | 1.00 | 31.40 |
| ATOM | 7140 | CH2 | TRP | B | 168 | 34.354 | 74.847 | 74.403 | 1.00 | 31.36 |
| ATOM | 7141 | N | ASN | B | 169 | 39.801 | 75.765 | 73.938 | 1.00 | 27.78 |
| ATOM | 7142 | CA | ASN | B | 169 | 39.676 | 76.441 | 72.636 | 1.00 | 28.42 |
| ATOM | 7143 | C | ASN | B | 169 | 40.469 | 77.741 | 72.552 | 1.00 | 27.41 |
| ATOM | 7144 | O | ASN | B | 169 | 40.011 | 78.742 | 71.976 | 1.00 | 26.62 |
| ATOM | 7145 | CB | ASN | B | 169 | 38.225 | 76.603 | 72.239 | 1.00 | 29.40 |
| ATOM | 7146 | CG | ASN | B | 169 | 37.588 | 75.267 | 71.844 | 1.00 | 31.42 |
| ATOM | 7147 | OD1 | ASN | B | 169 | 36.440 | 75.203 | 71.546 | 1.00 | 37.40 |
| ATOM | 7148 | ND2 | ASN | B | 169 | 38.351 | 74.219 | 71.858 | 1.00 | 33.37 |
| ATOM | 7149 | N | ASN | B | 170 | 41.674 | 77.710 | 73.127 | 1.00 | 25.57 |
| ATOM | 7150 | CA | ASN | B | 170 | 42.621 | 78.822 | 73.013 | 1.00 | 25.04 |
| ATOM | 7151 | C | ASN | B | 170 | 42.273 | 80.047 | 73.808 | 1.00 | 24.12 |
| ATOM | 7152 | O | ASN | B | 170 | 42.907 | 81.088 | 73.635 | 1.00 | 23.07 |
| ATOM | 7153 | CB | ASN | B | 170 | 42.833 | 79.246 | 71.542 | 1.00 | 25.51 |
| ATOM | 7154 | CG | ASN | B | 170 | 43.671 | 78.250 | 70.742 | 1.00 | 25.77 |
| ATOM | 7155 | OD1 | ASN | B | 170 | 43.598 | 77.047 | 70.953 | 1.00 | 26.32 |
| ATOM | 7156 | ND2 | ASN | B | 170 | 44.494 | 78.766 | 69.848 | 1.00 | 23.45 |
| ATOM | 7157 | N | ASP | B | 171 | 41.255 | 79.942 | 74.653 | 1.00 | 24.36 |
| ATOM | 7158 | CA | ASP | B | 171 | 40.908 | 81.025 | 75.535 | 1.00 | 25.01 |
| ATOM | 7159 | C | ASP | B | 171 | 41.116 | 80.625 | 76.984 | 1.00 | 25.77 |
| ATOM | 7160 | O | ASP | B | 171 | 41.075 | 79.450 | 77.342 | 1.00 | 25.33 |
| ATOM | 7161 | CB | ASP | B | 171 | 39.461 | 81.451 | 75.322 | 1.00 | 25.44 |
| ATOM | 7162 | CG | ASP | B | 171 | 39.282 | 82.398 | 74.138 | 1.00 | 26.23 |
| ATOM | 7163 | OD1 | ASP | B | 171 | 40.006 | 83.402 | 73.973 | 1.00 | 26.67 |
| ATOM | 7164 | OD2 | ASP | B | 171 | 38.398 | 82.225 | 73.322 | 1.00 | 31.72 |
| ATOM | 7165 | N | ILE | B | 172 | 41.265 | 81.642 | 77.828 | 1.00 | 26.70 |
| ATOM | 7166 | CA | ILE | B | 172 | 41.503 | 81.463 | 79.254 | 1.00 | 26.99 |
| ATOM | 7167 | C | ILE | B | 172 | 40.224 | 81.567 | 80.042 | 1.00 | 27.13 |
| ATOM | 7168 | O | ILE | B | 172 | 39.430 | 82.447 | 79.780 | 1.00 | 28.16 |
| ATOM | 7169 | CB | ILE | B | 172 | 42.443 | 82.577 | 79.733 | 1.00 | 27.38 |
| ATOM | 7170 | CG1 | ILE | B | 172 | 43.694 | 82.571 | 78.891 | 1.00 | 26.31 |
| ATOM | 7171 | CG2 | ILE | B | 172 | 42.748 | 82.479 | 81.268 | 1.00 | 27.37 |
| ATOM | 7172 | CD1 | ILE | B | 172 | 44.628 | 83.682 | 79.203 | 1.00 | 28.62 |
| ATOM | 7173 | N | TYR | B | 173 | 40.053 | 80.672 | 81.002 | 1.00 | 27.60 |
| ATOM | 7174 | CA | TYR | B | 173 | 38.939 | 80.689 | 81.944 | 1.00 | 29.16 |
| ATOM | 7175 | C | TYR | B | 173 | 39.487 | 80.600 | 83.336 | 1.00 | 29.79 |
| ATOM | 7176 | O | TYR | B | 173 | 40.559 | 80.022 | 83.545 | 1.00 | 27.70 |
| ATOM | 7177 | CB | TYR | B | 173 | 38.030 | 79.479 | 81.753 | 1.00 | 29.36 |
| ATOM | 7178 | CG | TYR | B | 173 | 37.340 | 79.485 | 80.431 | 1.00 | 29.61 |
| ATOM | 7179 | CD1 | TYR | B | 173 | 38.031 | 79.156 | 79.289 | 1.00 | 29.96 |
| ATOM | 7180 | CD2 | TYR | B | 173 | 36.007 | 79.862 | 80.321 | 1.00 | 30.68 |
| ATOM | 7181 | CE1 | TYR | B | 173 | 37.417 | 79.173 | 78.037 | 1.00 | 31.87 |
| ATOM | 7182 | CE2 | TYR | B | 173 | 35.357 | 79.866 | 79.070 | 1.00 | 30.15 |
| ATOM | 7183 | CZ | TYR | B | 173 | 36.081 | 79.514 | 77.935 | 1.00 | 29.94 |
| ATOM | 7184 | OH | TYR | B | 173 | 35.514 | 79.553 | 76.692 | 1.00 | 28.17 |
| ATOM | 7185 | N | VAL | B | 174 | 38.728 | 81.139 | 84.295 | 1.00 | 31.23 |
| ATOM | 7186 | CA | VAL | B | 174 | 39.111 | 81.074 | 85.697 | 1.00 | 33.00 |
| ATOM | 7187 | C | VAL | B | 174 | 37.983 | 80.538 | 86.587 | 1.00 | 34.12 |
| ATOM | 7188 | O | VAL | B | 174 | 36.816 | 80.912 | 86.412 | 1.00 | 34.28 |
| ATOM | 7189 | CB | VAL | B | 174 | 39.546 | 82.482 | 86.223 | 1.00 | 33.90 |
| ATOM | 7190 | CG1 | VAL | B | 174 | 39.769 | 82.455 | 87.736 | 1.00 | 34.58 |
| ATOM | 7191 | CG2 | VAL | B | 174 | 40.807 | 82.954 | 85.556 | 1.00 | 32.03 |
| ATOM | 7192 | N | LYS | B | 175 | 38.343 | 79.641 | 87.508 | 1.00 | 34.69 |
| ATOM | 7193 | CA | LYS | B | 175 | 37.430 | 79.108 | 88.522 | 1.00 | 35.51 |
| ATOM | 7194 | C | LYS | B | 175 | 37.968 | 79.489 | 89.872 | 1.00 | 36.26 |
| ATOM | 7195 | O | LYS | B | 175 | 39.108 | 79.152 | 90.204 | 1.00 | 36.45 |
| ATOM | 7196 | CB | LYS | B | 175 | 37.365 | 77.602 | 88.501 | 1.00 | 35.81 |
| ATOM | 7197 | CG | LYS | B | 175 | 36.352 | 77.025 | 87.516 | 1.00 | 38.80 |
| ATOM | 7198 | CD | LYS | B | 175 | 36.367 | 75.511 | 87.545 | 1.00 | 40.55 |
| ATOM | 7199 | CE | LYS | B | 175 | 34.990 | 74.962 | 87.372 | 1.00 | 42.69 |
| ATOM | 7200 | NZ | LYS | B | 175 | 34.425 | 74.585 | 88.683 | 1.00 | 43.10 |
| ATOM | 7201 | N | ILE | B | 176 | 37.159 | 80.204 | 90.641 | 1.00 | 37.18 |
| ATOM | 7202 | CA | ILE | B | 176 | 37.528 | 80.636 | 91.986 | 1.00 | 37.14 |
| ATOM | 7203 | C | ILE | B | 176 | 37.298 | 79.471 | 92.909 | 1.00 | 37.45 |
| ATOM | 7204 | O | ILE | B | 176 | 38.066 | 79.265 | 93.823 | 1.00 | 37.82 |
| ATOM | 7205 | CB | ILE | B | 176 | 36.680 | 81.832 | 92.399 | 1.00 | 38.10 |
| ATOM | 7206 | CG1 | ILE | B | 176 | 37.003 | 83.028 | 91.494 | 1.00 | 38.17 |
| ATOM | 7207 | CG2 | ILE | B | 176 | 36.865 | 82.178 | 93.913 | 1.00 | 38.95 |
| ATOM | 7208 | CD1 | ILE | B | 176 | 38.466 | 83.421 | 91.501 | 1.00 | 38.95 |
| ATOM | 7209 | N | GLU | B | 177 | 36.251 | 78.680 | 92.681 | 1.00 | 37.61 |
| ATOM | 7210 | CA | GLU | B | 177 | 36.050 | 77.476 | 93.498 | 1.00 | 37.49 |
| ATOM | 7211 | C | GLU | B | 177 | 35.825 | 76.353 | 92.567 | 1.00 | 36.65 |
| ATOM | 7212 | O | GLU | B | 177 | 35.155 | 76.515 | 91.548 | 1.00 | 36.82 |
| ATOM | 7213 | CB | GLU | B | 177 | 34.821 | 77.561 | 94.427 | 1.00 | 38.49 |
| ATOM | 7214 | CG | GLU | B | 177 | 34.924 | 78.567 | 95.558 | 1.00 | 39.23 |
| ATOM | 7215 | CD | GLU | B | 177 | 36.037 | 78.220 | 96.501 | 1.00 | 41.78 |
| ATOM | 7216 | OE1 | GLU | B | 177 | 36.325 | 77.003 | 96.632 | 1.00 | 41.29 |
| ATOM | 7217 | OE2 | GLU | B | 177 | 36.617 | 79.160 | 97.099 | 1.00 | 42.97 |
| ATOM | 7218 | N | PRO | B | 178 | 36.378 | 75.208 | 92.899 | 1.00 | 36.33 |
| ATOM | 7219 | CA | PRO | B | 178 | 36.235 | 74.018 | 92.057 | 1.00 | 36.94 |
| ATOM | 7220 | C | PRO | B | 178 | 34.832 | 73.724 | 91.518 | 1.00 | 37.41 |
| ATOM | 7221 | O | PRO | B | 178 | 34.718 | 73.336 | 90.354 | 1.00 | 37.02 |
| ATOM | 7222 | CB | PRO | B | 178 | 36.723 | 72.900 | 92.968 | 1.00 | 36.73 |
| ATOM | 7223 | CG | PRO | B | 178 | 37.755 | 73.585 | 93.833 | 1.00 | 36.47 |
| ATOM | 7224 | CD | PRO | B | 178 | 37.212 | 74.954 | 94.079 | 1.00 | 36.06 |
| ATOM | 7225 | N | ASN | B | 179 | 33.781 | 73.905 | 92.303 | 1.00 | 38.31 |
| ATOM | 7226 | CA | ASN | B | 179 | 32.448 | 73.518 | 91.833 | 1.00 | 39.69 |
| ATOM | 7227 | C | ASN | B | 179 | 31.625 | 74.684 | 91.289 | 1.00 | 40.15 |
| ATOM | 7228 | O | ASN | B | 179 | 30.432 | 74.529 | 91.004 | 1.00 | 39.85 |
| ATOM | 7229 | CB | ASN | B | 179 | 31.676 | 72.810 | 92.942 | 1.00 | 40.15 |
| ATOM | 7230 | CG | ASN | B | 179 | 31.533 | 73.674 | 94.150 | 1.00 | 42.09 |
| ATOM | 7231 | OD1 | ASN | B | 179 | 32.178 | 74.721 | 94.250 | 1.00 | 43.38 |
| ATOM | 7232 | ND2 | ASN | B | 179 | 30.710 | 73.250 | 95.085 | 1.00 | 47.94 |
| ATOM | 7233 | N | LEU | B | 180 | 32.249 | 75.844 | 91.112 | 1.00 | 40.10 |
| ATOM | 7234 | CA | LEU | B | 180 | 31.520 | 76.966 | 90.547 | 1.00 | 40.56 |
| ATOM | 7235 | C | LEU | B | 180 | 31.829 | 77.257 | 89.073 | 1.00 | 39.05 |
| ATOM | 7236 | O | LEU | B | 180 | 32.855 | 76.849 | 88.543 | 1.00 | 38.39 |
| ATOM | 7237 | CB | LEU | B | 180 | 31.756 | 78.219 | 91.388 | 1.00 | 41.21 |
| ATOM | 7238 | CG | LEU | B | 180 | 30.847 | 78.321 | 92.619 | 1.00 | 45.88 |
| ATOM | 7239 | CD1 | LEU | B | 180 | 29.368 | 77.967 | 92.302 | 1.00 | 48.11 |
| ATOM | 7240 | CD2 | LEU | B | 180 | 31.318 | 77.406 | 93.714 | 1.00 | 49.70 |
| ATOM | 7241 | N | PRO | B | 181 | 30.898 | 77.926 | 88.410 | 1.00 | 37.96 |
| ATOM | 7242 | CA | PRO | B | 181 | 31.084 | 78.337 | 87.028 | 1.00 | 37.59 |
| ATOM | 7243 | C | PRO | B | 181 | 32.383 | 79.071 | 86.845 | 1.00 | 37.17 |
| ATOM | 7244 | O | PRO | B | 181 | 32.809 | 79.808 | 87.757 | 1.00 | 37.40 |
| ATOM | 7245 | CB | PRO | B | 181 | 29.931 | 79.331 | 86.799 | 1.00 | 37.51 |
| ATOM | 7246 | CG | PRO | B | 181 | 28.822 | 78.824 | 87.747 | 1.00 | 38.58 |
| ATOM | 7247 | CD | PRO | B | 181 | 29.563 | 78.287 | 88.921 | 1.00 | 38.00 |
| ATOM | 7248 | N | SER | B | 182 | 32.991 | 78.923 | 85.680 | 1.00 | 35.56 |
| ATOM | 7249 | CA | SER | B | 182 | 34.188 | 79.663 | 85.416 | 1.00 | 35.61 |
| ATOM | 7250 | C | SER | B | 182 | 33.842 | 81.037 | 84.885 | 1.00 | 35.09 |
| ATOM | 7251 | O | SER | B | 182 | 32.775 | 81.257 | 84.368 | 1.00 | 34.78 |
| ATOM | 7252 | CB | SER | B | 182 | 35.081 | 78.928 | 84.406 | 1.00 | 35.34 |
| ATOM | 7253 | OG | SER | B | 182 | 34.295 | 78.443 | 83.364 | 1.00 | 37.20 |
| ATOM | 7254 | N | TYR | B | 183 | 34.780 | 81.958 | 85.037 | 1.00 | 34.82 |
| ATOM | 7255 | CA | TYR | B | 183 | 34.688 | 83.262 | 84.450 | 1.00 | 34.50 |
| ATOM | 7256 | C | TYR | B | 183 | 35.520 | 83.177 | 83.193 | 1.00 | 33.68 |
| ATOM | 7257 | O | TYR | B | 183 | 36.659 | 82.720 | 83.241 | 1.00 | 33.67 |
| ATOM | 7258 | CB | TYR | B | 183 | 35.278 | 84.328 | 85.396 | 1.00 | 35.09 |
| ATOM | 7259 | CG | TYR | B | 183 | 34.510 | 84.382 | 86.676 | 1.00 | 34.39 |
| ATOM | 7260 | CD1 | TYR | B | 183 | 34.860 | 83.564 | 87.718 | 1.00 | 34.06 |
| ATOM | 7261 | CD2 | TYR | B | 183 | 33.369 | 85.201 | 86.807 | 1.00 | 34.61 |
| ATOM | 7262 | CE1 | TYR | B | 183 | 34.154 | 83.556 | 88.869 | 1.00 | 36.35 |
| ATOM | 7263 | CE2 | TYR | B | 183 | 32.648 | 85.219 | 87.973 | 1.00 | 35.36 |
| ATOM | 7264 | CZ | TYR | B | 183 | 33.040 | 84.377 | 89.003 | 1.00 | 37.94 |
| ATOM | 7265 | OH | TYR | B | 183 | 32.365 | 84.337 | 90.187 | 1.00 | 40.21 |
| ATOM | 7266 | N | ARG | B | 184 | 34.949 | 83.589 | 82.063 | 1.00 | 32.36 |
| ATOM | 7267 | CA | ARG | B | 184 | 35.686 | 83.605 | 80.804 | 1.00 | 32.58 |
| ATOM | 7268 | C | ARG | B | 184 | 36.505 | 84.877 | 80.728 | 1.00 | 31.81 |
| ATOM | 7269 | O | ARG | B | 184 | 35.964 | 85.932 | 80.829 | 1.00 | 33.62 |
| ATOM | 7270 | CB | ARG | B | 184 | 34.723 | 83.527 | 79.622 | 1.00 | 32.04 |
| ATOM | 7271 | CG | ARG | B | 184 | 35.402 | 83.574 | 78.268 | 1.00 | 33.23 |
| ATOM | 7272 | CD | ARG | B | 184 | 34.642 | 82.835 | 77.197 | 1.00 | 32.80 |
| ATOM | 7273 | NE | ARG | B | 184 | 35.371 | 82.777 | 75.937 | 1.00 | 34.26 |
| ATOM | 7274 | CZ | ARG | B | 184 | 34.838 | 82.391 | 74.773 | 1.00 | 32.66 |
| ATOM | 7275 | NH1 | ARG | B | 184 | 33.574 | 82.000 | 74.699 | 1.00 | 32.08 |
| ATOM | 7276 | NH2 | ARG | B | 184 | 35.573 | 82.370 | 73.698 | 1.00 | 27.82 |
| ATOM | 7277 | N | ILE | B | 185 | 37.805 | 84.791 | 80.511 | 1.00 | 31.72 |
| ATOM | 7278 | CA | ILE | B | 185 | 38.656 | 85.974 | 80.487 | 1.00 | 30.57 |
| ATOM | 7279 | C | ILE | B | 185 | 38.925 | 86.496 | 79.087 | 1.00 | 30.73 |
| ATOM | 7280 | O | ILE | B | 185 | 38.971 | 87.704 | 78.900 | 1.00 | 31.11 |
| ATOM | 7281 | CB | ILE | B | 185 | 40.019 | 85.616 | 81.148 | 1.00 | 30.53 |
| ATOM | 7282 | CG1 | ILE | B | 185 | 39.806 | 84.939 | 82.501 | 1.00 | 31.03 |
| ATOM | 7283 | CG2 | ILE | B | 185 | 40.951 | 86.815 | 81.252 | 1.00 | 28.61 |
| ATOM | 7284 | CD1 | ILE | B | 185 | 39.150 | 85.829 | 83.580 | 1.00 | 30.91 |
| ATOM | 7285 | N | THR | B | 186 | 39.202 | 85.604 | 78.127 | 1.00 | 29.74 |
| ATOM | 7286 | CA | THR | B | 186 | 39.437 | 86.022 | 76.753 | 1.00 | 28.74 |
| ATOM | 7287 | C | THR | B | 186 | 38.360 | 85.490 | 75.845 | 1.00 | 29.54 |
| ATOM | 7288 | O | THR | B | 186 | 37.757 | 84.466 | 76.158 | 1.00 | 28.68 |
| ATOM | 7289 | CB | THR | B | 186 | 40.792 | 85.552 | 76.208 | 1.00 | 29.21 |
| ATOM | 7290 | OG1 | THR | B | 186 | 40.892 | 84.098 | 76.217 | 1.00 | 24.81 |
| ATOM | 7291 | CG2 | THR | B | 186 | 41.922 | 86.139 | 77.084 | 1.00 | 28.72 |
| ATOM | 7292 | N | TRP | B | 187 | 38.169 | 86.169 | 74.710 | 1.00 | 30.37 |
| ATOM | 7293 | CA | TRP | B | 187 | 37.138 | 85.801 | 73.715 | 1.00 | 31.00 |
| ATOM | 7294 | C | TRP | B | 187 | 37.680 | 85.721 | 72.313 | 1.00 | 30.96 |
| ATOM | 7295 | O | TRP | B | 187 | 36.917 | 85.495 | 71.378 | 1.00 | 31.78 |
| ATOM | 7296 | CB | TRP | B | 187 | 36.000 | 86.840 | 73.734 | 1.00 | 31.13 |
| ATOM | 7297 | CG | TRP | B | 187 | 35.306 | 86.910 | 75.049 | 1.00 | 33.04 |
| ATOM | 7298 | CD1 | TRP | B | 187 | 35.733 | 87.586 | 76.161 | 1.00 | 34.11 |
| ATOM | 7299 | CD2 | TRP | B | 187 | 34.077 | 86.271 | 75.420 | 1.00 | 37.07 |
| ATOM | 7300 | NE1 | TRP | B | 187 | 34.841 | 87.400 | 77.191 | 1.00 | 36.25 |
| ATOM | 7301 | CE2 | TRP | B | 187 | 33.816 | 86.600 | 76.757 | 1.00 | 37.19 |
| ATOM | 7302 | CE3 | TRP | B | 187 | 33.173 | 85.440 | 74.756 | 1.00 | 41.52 |
| ATOM | 7303 | CZ2 | TRP | B | 187 | 32.696 | 86.135 | 77.441 | 1.00 | 41.86 |
| ATOM | 7304 | CZ3 | TRP | B | 187 | 32.047 | 84.987 | 75.435 | 1.00 | 41.77 |
| ATOM | 7305 | CH2 | TRP | B | 187 | 31.823 | 85.338 | 76.768 | 1.00 | 42.71 |
| ATOM | 7306 | N | THR | B | 188 | 38.992 | 85.913 | 72.155 | 1.00 | 30.36 |
| ATOM | 7307 | CA | THR | B | 188 | 39.627 | 85.973 | 70.866 | 1.00 | 30.00 |
| ATOM | 7308 | C | THR | B | 188 | 40.291 | 84.686 | 70.424 | 1.00 | 30.49 |
| ATOM | 7309 | O | THR | B | 188 | 40.908 | 84.651 | 69.373 | 1.00 | 30.70 |
| ATOM | 7310 | CB | THR | B | 188 | 40.730 | 87.063 | 70.897 | 1.00 | 30.29 |
| ATOM | 7311 | OG1 | THR | B | 188 | 41.580 | 86.859 | 72.032 | 1.00 | 27.37 |
| ATOM | 7312 | CG2 | THR | B | 188 | 40.137 | 88.460 | 71.127 | 1.00 | 30.50 |
| ATOM | 7313 | N | GLY | B | 189 | 40.236 | 83.654 | 71.241 | 1.00 | 30.99 |
| ATOM | 7314 | CA | GLY | B | 189 | 40.882 | 82.406 | 70.897 | 1.00 | 31.24 |
| ATOM | 7315 | C | GLY | B | 189 | 40.409 | 81.863 | 69.560 | 1.00 | 32.02 |
| ATOM | 7316 | O | GLY | B | 189 | 39.223 | 81.866 | 69.272 | 1.00 | 30.37 |
| ATOM | 7317 | N | LYS | B | 190 | 41.354 | 81.388 | 68.757 | 1.00 | 33.66 |
| ATOM | 7318 | CA | LYS | B | 190 | 41.038 | 80.824 | 67.452 | 1.00 | 35.04 |
| ATOM | 7319 | C | LYS | B | 190 | 42.166 | 79.864 | 67.015 | 1.00 | 34.99 |
| ATOM | 7320 | O | LYS | B | 190 | 43.356 | 80.221 | 66.913 | 1.00 | 34.01 |
| ATOM | 7321 | CB | LYS | B | 190 | 40.775 | 81.942 | 66.429 | 1.00 | 35.38 |
| ATOM | 7322 | CG | LYS | B | 190 | 40.545 | 81.428 | 65.018 | 1.00 | 39.53 |
| ATOM | 7323 | CD | LYS | B | 190 | 39.917 | 82.506 | 64.075 | 1.00 | 44.37 |
| ATOM | 7324 | CE | LYS | B | 190 | 38.972 | 81.861 | 63.027 | 1.00 | 45.44 |
| ATOM | 7325 | NZ | LYS | B | 190 | 37.900 | 82.816 | 62.564 | 1.00 | 48.69 |
| ATOM | 7326 | N | GLU | B | 191 | 41.757 | 78.640 | 66.750 | 1.00 | 34.96 |
| ATOM | 7327 | CA | GLU | B | 191 | 42.682 | 77.580 | 66.441 | 1.00 | 35.61 |
| ATOM | 7328 | C | GLU | B | 191 | 43.711 | 78.047 | 65.399 | 1.00 | 34.46 |
| ATOM | 7329 | O | GLU | B | 191 | 43.358 | 78.723 | 64.444 | 1.00 | 33.49 |
| ATOM | 7330 | CB | GLU | B | 191 | 41.892 | 76.365 | 65.968 | 1.00 | 36.18 |
| ATOM | 7331 | CG | GLU | B | 191 | 42.602 | 75.042 | 66.131 | 1.00 | 41.19 |
| ATOM | 7332 | CD | GLU | B | 191 | 41.692 | 73.861 | 65.794 | 1.00 | 45.56 |
| ATOM | 7333 | OE1 | GLU | B | 191 | 40.903 | 73.401 | 66.674 | 1.00 | 48.35 |
| ATOM | 7334 | OE2 | GLU | B | 191 | 41.745 | 73.415 | 64.639 | 1.00 | 44.81 |
| ATOM | 7335 | N | ASP | B | 192 | 44.975 | 77.691 | 65.646 | 1.00 | 32.21 |
| ATOM | 7336 | CA | ASP | B | 192 | 46.138 | 78.031 | 64.851 | 1.00 | 31.35 |
| ATOM | 7337 | C | ASP | B | 192 | 46.318 | 79.485 | 64.541 | 1.00 | 29.99 |
| ATOM | 7338 | O | ASP | B | 192 | 47.166 | 79.810 | 63.752 | 1.00 | 29.26 |
| ATOM | 7339 | CB | ASP | B | 192 | 46.128 | 77.279 | 63.514 | 1.00 | 32.07 |
| ATOM | 7340 | CG | ASP | B | 192 | 46.167 | 75.799 | 63.694 | 1.00 | 33.42 |
| ATOM | 7341 | OD1 | ASP | B | 192 | 46.877 | 75.306 | 64.610 | 1.00 | 36.83 |
| ATOM | 7342 | OD2 | ASP | B | 192 | 45.483 | 75.055 | 62.999 | 1.00 | 34.81 |
| ATOM | 7343 | N | ILE | B | 193 | 45.587 | 80.375 | 65.181 | 1.00 | 29.66 |
| ATOM | 7344 | CA | ILE | B | 193 | 45.702 | 81.777 | 64.823 | 1.00 | 29.80 |
| ATOM | 7345 | C | ILE | B | 193 | 45.914 | 82.678 | 66.028 | 1.00 | 28.95 |
| ATOM | 7346 | O | ILE | B | 193 | 46.898 | 83.399 | 66.073 | 1.00 | 28.62 |
| ATOM | 7347 | CB | ILE | B | 193 | 44.461 | 82.220 | 64.029 | 1.00 | 30.62 |
| ATOM | 7348 | CG1 | ILE | B | 193 | 44.373 | 81.433 | 62.728 | 1.00 | 32.28 |
| ATOM | 7349 | CG2 | ILE | B | 193 | 44.520 | 83.749 | 63.742 | 1.00 | 33.14 |
| ATOM | 7350 | CD1 | ILE | B | 193 | 43.175 | 81.840 | 61.855 | 1.00 | 36.86 |
| ATOM | 7351 | N | ILE | B | 194 | 44.987 | 82.671 | 66.983 | 1.00 | 27.43 |
| ATOM | 7352 | CA | ILE | B | 194 | 45.150 | 83.474 | 68.181 | 1.00 | 27.39 |
| ATOM | 7353 | C | ILE | B | 194 | 45.266 | 82.551 | 69.399 | 1.00 | 26.62 |
| ATOM | 7354 | O | ILE | B | 194 | 44.393 | 81.732 | 69.652 | 1.00 | 27.07 |
| ATOM | 7355 | CB | ILE | B | 194 | 43.959 | 84.369 | 68.414 | 1.00 | 27.86 |
| ATOM | 7356 | CG1 | ILE | B | 194 | 43.729 | 85.381 | 67.267 | 1.00 | 27.98 |
| ATOM | 7357 | CG2 | ILE | B | 194 | 44.093 | 85.053 | 69.769 | 1.00 | 29.81 |
| ATOM | 7358 | CD1 | ILE | B | 194 | 44.845 | 86.329 | 66.950 | 1.00 | 28.45 |
| ATOM | 7359 | N | TYR | B | 195 | 46.318 | 82.722 | 70.182 | 1.00 | 25.71 |
| ATOM | 7360 | CA | TYR | B | 195 | 46.555 | 81.858 | 71.340 | 1.00 | 25.32 |
| ATOM | 7361 | C | TYR | B | 195 | 46.614 | 82.683 | 72.678 | 1.00 | 24.78 |
| ATOM | 7362 | O | TYR | B | 195 | 47.474 | 83.517 | 72.868 | 1.00 | 24.30 |
| ATOM | 7363 | CB | TYR | B | 195 | 47.889 | 81.158 | 71.161 | 1.00 | 25.48 |
| ATOM | 7364 | CG | TYR | B | 195 | 48.147 | 80.261 | 69.958 | 1.00 | 25.25 |
| ATOM | 7365 | CD1 | TYR | B | 195 | 48.509 | 80.777 | 68.722 | 1.00 | 28.88 |
| ATOM | 7366 | CD2 | TYR | B | 195 | 48.154 | 78.870 | 70.095 | 1.00 | 28.51 |
| ATOM | 7367 | CE1 | TYR | B | 195 | 48.798 | 79.928 | 67.627 | 1.00 | 27.62 |
| ATOM | 7368 | CE2 | TYR | B | 195 | 48.470 | 78.012 | 69.003 | 1.00 | 25.69 |
| ATOM | 7369 | CZ | TYR | B | 195 | 48.784 | 78.552 | 67.803 | 1.00 | 28.86 |
| ATOM | 7370 | OH | TYR | B | 195 | 49.089 | 77.704 | 66.769 | 1.00 | 32.98 |
| ATOM | 7371 | N | ASN | B | 196 | 45.692 | 82.444 | 73.598 | 1.00 | 24.41 |
| ATOM | 7372 | CA | ASN | B | 196 | 45.674 | 83.183 | 74.834 | 1.00 | 24.30 |
| ATOM | 7373 | C | ASN | B | 196 | 46.053 | 82.200 | 75.937 | 1.00 | 24.78 |
| ATOM | 7374 | O | ASN | B | 196 | 45.365 | 81.220 | 76.188 | 1.00 | 23.78 |
| ATOM | 7375 | CB | ASN | B | 196 | 44.295 | 83.796 | 75.145 | 1.00 | 23.89 |
| ATOM | 7376 | CG | ASN | B | 196 | 43.853 | 84.855 | 74.119 | 1.00 | 23.79 |
| ATOM | 7377 | OD1 | ASN | B | 196 | 44.404 | 85.941 | 74.062 | 1.00 | 24.66 |
| ATOM | 7378 | ND2 | ASN | B | 196 | 42.810 | 84.547 | 73.364 | 1.00 | 22.39 |
| ATOM | 7379 | N | GLY | B | 197 | 47.150 | 82.476 | 76.599 | 1.00 | 25.39 |
| ATOM | 7380 | CA | GLY | B | 197 | 47.525 | 81.669 | 77.745 | 1.00 | 26.17 |
| ATOM | 7381 | C | GLY | B | 197 | 48.212 | 80.378 | 77.422 | 1.00 | 25.33 |
| ATOM | 7382 | O | GLY | B | 197 | 48.519 | 79.637 | 78.356 | 1.00 | 26.86 |
| ATOM | 7383 | N | ILE | B | 198 | 48.366 | 80.086 | 76.132 | 1.00 | 24.30 |
| ATOM | 7384 | CA | ILE | B | 198 | 49.213 | 79.004 | 75.671 | 1.00 | 23.97 |
| ATOM | 7385 | C | ILE | B | 198 | 50.078 | 79.547 | 74.521 | 1.00 | 24.02 |
| ATOM | 7386 | O | ILE | B | 198 | 49.754 | 80.607 | 73.954 | 1.00 | 24.44 |
| ATOM | 7387 | CB | ILE | B | 198 | 48.418 | 77.790 | 75.190 | 1.00 | 24.16 |
| ATOM | 7388 | CG1 | ILE | B | 198 | 47.310 | 78.236 | 74.222 | 1.00 | 24.52 |
| ATOM | 7389 | CG2 | ILE | B | 198 | 47.891 | 76.975 | 76.389 | 1.00 | 21.53 |
| ATOM | 7390 | CD1 | ILE | B | 198 | 46.628 | 77.132 | 73.499 | 1.00 | 24.60 |
| ATOM | 7391 | N | THR | B | 199 | 51.169 | 78.836 | 74.224 | 1.00 | 23.15 |
| ATOM | 7392 | CA | THR | B | 199 | 52.122 | 79.176 | 73.192 | 1.00 | 23.42 |
| ATOM | 7393 | C | THR | B | 199 | 51.810 | 78.461 | 71.870 | 1.00 | 23.55 |
| ATOM | 7394 | O | THR | B | 199 | 51.195 | 77.390 | 71.840 | 1.00 | 24.28 |
| ATOM | 7395 | CB | THR | B | 199 | 53.529 | 78.742 | 73.589 | 1.00 | 23.42 |
| ATOM | 7396 | OG1 | THR | B | 199 | 53.536 | 77.336 | 73.944 | 1.00 | 22.03 |
| ATOM | 7397 | CG2 | THR | B | 199 | 54.047 | 79.528 | 74.858 | 1.00 | 23.33 |
| ATOM | 7398 | N | ASP | B | 200 | 52.223 | 79.098 | 70.786 | 1.00 | 23.94 |
| ATOM | 7399 | CA | ASP | B | 200 | 52.202 | 78.499 | 69.449 | 1.00 | 24.01 |
| ATOM | 7400 | C | ASP | B | 200 | 53.425 | 77.600 | 69.345 | 1.00 | 23.87 |
| ATOM | 7401 | O | ASP | B | 200 | 54.156 | 77.393 | 70.346 | 1.00 | 23.48 |
| ATOM | 7402 | CB | ASP | B | 200 | 52.193 | 79.595 | 68.384 | 1.00 | 24.16 |
| ATOM | 7403 | CG | ASP | B | 200 | 53.550 | 80.189 | 68.127 | 1.00 | 24.91 |
| ATOM | 7404 | OD1 | ASP | B | 200 | 54.429 | 80.171 | 69.033 | 1.00 | 20.58 |
| ATOM | 7405 | OD2 | ASP | B | 200 | 53.835 | 80.661 | 67.004 | 1.00 | 29.25 |
| ATOM | 7406 | N | TRP | B | 201 | 53.697 | 77.095 | 68.150 | 1.00 | 23.54 |
| ATOM | 7407 | CA | TRP | B | 201 | 54.761 | 76.106 | 67.970 | 1.00 | 23.22 |
| ATOM | 7408 | C | TRP | B | 201 | 56.192 | 76.577 | 68.302 | 1.00 | 23.29 |
| ATOM | 7409 | O | TRP | B | 201 | 56.952 | 75.851 | 68.941 | 1.00 | 24.13 |
| ATOM | 7410 | CB | TRP | B | 201 | 54.770 | 75.533 | 66.543 | 1.00 | 22.81 |
| ATOM | 7411 | CG | TRP | B | 201 | 55.590 | 74.325 | 66.446 | 1.00 | 21.22 |
| ATOM | 7412 | CD1 | TRP | B | 201 | 55.150 | 73.069 | 66.497 | 1.00 | 21.30 |
| ATOM | 7413 | CD2 | TRP | B | 201 | 57.017 | 74.248 | 66.374 | 1.00 | 19.24 |
| ATOM | 7414 | NE1 | TRP | B | 201 | 56.196 | 72.182 | 66.423 | 1.00 | 20.03 |
| ATOM | 7415 | CE2 | TRP | B | 201 | 57.360 | 72.884 | 66.346 | 1.00 | 19.91 |
| ATOM | 7416 | CE3 | TRP | B | 201 | 58.031 | 75.183 | 66.257 | 1.00 | 22.09 |
| ATOM | 7417 | CZ2 | TRP | B | 201 | 58.672 | 72.429 | 66.245 | 1.00 | 20.94 |
| ATOM | 7418 | CZ3 | TRP | B | 201 | 59.367 | 74.732 | 66.168 | 1.00 | 22.14 |
| ATOM | 7419 | CH2 | TRP | B | 201 | 59.664 | 73.368 | 66.164 | 1.00 | 21.14 |
| ATOM | 7420 | N | VAL | B | 202 | 56.585 | 77.752 | 67.853 | 1.00 | 22.90 |
| ATOM | 7421 | CA | VAL | B | 202 | 57.938 | 78.137 | 68.096 | 1.00 | 23.28 |
| ATOM | 7422 | C | VAL | B | 202 | 58.149 | 78.500 | 69.531 | 1.00 | 22.71 |
| ATOM | 7423 | O | VAL | B | 202 | 59.209 | 78.256 | 70.034 | 1.00 | 21.85 |
| ATOM | 7424 | CB | VAL | B | 202 | 58.464 | 79.422 | 67.415 | 1.00 | 23.30 |
| ATOM | 7425 | CG1 | VAL | B | 202 | 59.617 | 79.138 | 66.585 | 1.00 | 25.16 |
| ATOM | 7426 | CG2 | VAL | B | 202 | 57.452 | 80.287 | 66.869 | 1.00 | 24.41 |
| ATOM | 7427 | N | TYR | B | 203 | 57.207 | 79.244 | 70.097 | 1.00 | 22.28 |
| ATOM | 7428 | CA | TYR | B | 203 | 57.253 | 79.597 | 71.521 | 1.00 | 22.69 |
| ATOM | 7429 | C | TYR | B | 203 | 57.235 | 78.382 | 72.441 | 1.00 | 23.05 |
| ATOM | 7430 | O | TYR | B | 203 | 57.927 | 78.381 | 73.477 | 1.00 | 23.40 |
| ATOM | 7431 | CB | TYR | B | 203 | 56.134 | 80.561 | 71.893 | 1.00 | 21.84 |
| ATOM | 7432 | CG | TYR | B | 203 | 56.503 | 82.031 | 71.761 | 1.00 | 23.10 |
| ATOM | 7433 | CD1 | TYR | B | 203 | 56.306 | 82.719 | 70.585 | 1.00 | 23.62 |
| ATOM | 7434 | CD2 | TYR | B | 203 | 57.086 | 82.726 | 72.834 | 1.00 | 24.96 |
| ATOM | 7435 | CE1 | TYR | B | 203 | 56.674 | 84.078 | 70.473 | 1.00 | 25.42 |
| ATOM | 7436 | CE2 | TYR | B | 203 | 57.466 | 84.042 | 72.728 | 1.00 | 25.18 |
| ATOM | 7437 | CZ | TYR | B | 203 | 57.239 | 84.720 | 71.559 | 1.00 | 27.71 |
| ATOM | 7438 | OH | TYR | B | 203 | 57.591 | 86.039 | 71.495 | 1.00 | 28.35 |
| ATOM | 7439 | N | GLU | B | 204 | 56.503 | 77.326 | 72.081 | 1.00 | 22.52 |
| ATOM | 7440 | CA | GLU | B | 204 | 56.475 | 76.160 | 72.952 | 1.00 | 22.72 |
| ATOM | 7441 | C | GLU | B | 204 | 57.839 | 75.592 | 72.942 | 1.00 | 23.42 |
| ATOM | 7442 | O | GLU | B | 204 | 58.439 | 75.359 | 73.972 | 1.00 | 24.10 |
| ATOM | 7443 | CB | GLU | B | 204 | 55.493 | 75.067 | 72.477 | 1.00 | 22.71 |
| ATOM | 7444 | CG | GLU | B | 204 | 55.757 | 73.681 | 73.076 | 1.00 | 21.04 |
| ATOM | 7445 | CD | GLU | B | 204 | 54.720 | 72.602 | 72.726 | 1.00 | 19.46 |
| ATOM | 7446 | OE1 | GLU | B | 204 | 53.661 | 72.876 | 72.147 | 1.00 | 20.21 |
| ATOM | 7447 | OE2 | GLU | B | 204 | 54.950 | 71.438 | 73.026 | 1.00 | 18.42 |
| ATOM | 7448 | N | GLU | B | 205 | 58.338 | 75.393 | 71.734 | 1.00 | 23.62 |
| ATOM | 7449 | CA | GLU | B | 205 | 59.547 | 74.638 | 71.544 | 1.00 | 23.72 |
| ATOM | 7450 | C | GLU | B | 205 | 60.834 | 75.418 | 71.788 | 1.00 | 24.81 |
| ATOM | 7451 | O | GLU | B | 205 | 61.733 | 74.886 | 72.425 | 1.00 | 23.39 |
| ATOM | 7452 | CB | GLU | B | 205 | 59.516 | 74.049 | 70.129 | 1.00 | 23.14 |
| ATOM | 7453 | CG | GLU | B | 205 | 60.709 | 73.231 | 69.668 | 1.00 | 23.21 |
| ATOM | 7454 | CD | GLU | B | 205 | 61.026 | 71.992 | 70.499 | 1.00 | 24.56 |
| ATOM | 7455 | OE1 | GLU | B | 205 | 60.159 | 71.490 | 71.251 | 1.00 | 24.88 |
| ATOM | 7456 | OE2 | GLU | B | 205 | 62.186 | 71.535 | 70.418 | 1.00 | 22.25 |
| ATOM | 7457 | N | GLU | B | 206 | 60.946 | 76.639 | 71.262 | 1.00 | 25.08 |
| ATOM | 7458 | CA | GLU | B | 206 | 62.201 | 77.361 | 71.344 | 1.00 | 26.18 |
| ATOM | 7459 | C | GLU | B | 206 | 62.268 | 78.488 | 72.381 | 1.00 | 27.62 |
| ATOM | 7460 | O | GLU | B | 206 | 63.314 | 78.744 | 72.938 | 1.00 | 29.23 |
| ATOM | 7461 | CB | GLU | B | 206 | 62.523 | 77.961 | 69.973 | 1.00 | 26.21 |
| ATOM | 7462 | CG | GLU | B | 206 | 62.593 | 76.964 | 68.847 | 1.00 | 27.07 |
| ATOM | 7463 | CD | GLU | B | 206 | 63.755 | 75.980 | 68.948 | 1.00 | 27.64 |
| ATOM | 7464 | OE1 | GLU | B | 206 | 64.470 | 76.004 | 69.936 | 1.00 | 28.80 |
| ATOM | 7465 | OE2 | GLU | B | 206 | 63.914 | 75.143 | 68.037 | 1.00 | 25.55 |
| ATOM | 7466 | N | VAL | B | 207 | 61.188 | 79.198 | 72.641 | 1.00 | 27.27 |
| ATOM | 7467 | CA | VAL | B | 207 | 61.319 | 80.286 | 73.586 | 1.00 | 27.65 |
| ATOM | 7468 | C | VAL | B | 207 | 60.983 | 79.914 | 75.030 | 1.00 | 26.94 |
| ATOM | 7469 | O | VAL | B | 207 | 61.803 | 80.149 | 75.933 | 1.00 | 25.85 |
| ATOM | 7470 | CB | VAL | B | 207 | 60.525 | 81.538 | 73.137 | 1.00 | 28.03 |
| ATOM | 7471 | CG1 | VAL | B | 207 | 60.995 | 82.724 | 73.892 | 1.00 | 27.28 |
| ATOM | 7472 | CG2 | VAL | B | 207 | 60.765 | 81.806 | 71.645 | 1.00 | 27.49 |
| ATOM | 7473 | N | PHE | B | 208 | 59.836 | 79.306 | 75.281 | 1.00 | 26.63 |
| ATOM | 7474 | CA | PHE | B | 208 | 59.503 | 79.017 | 76.672 | 1.00 | 26.90 |
| ATOM | 7475 | C | PHE | B | 208 | 59.844 | 77.614 | 77.116 | 1.00 | 26.57 |
| ATOM | 7476 | O | PHE | B | 208 | 59.875 | 77.383 | 78.298 | 1.00 | 25.78 |
| ATOM | 7477 | CB | PHE | B | 208 | 57.989 | 79.174 | 77.000 | 1.00 | 27.24 |
| ATOM | 7478 | CG | PHE | B | 208 | 57.488 | 80.562 | 76.986 | 1.00 | 27.03 |
| ATOM | 7479 | CD1 | PHE | B | 208 | 58.328 | 81.623 | 76.747 | 1.00 | 28.32 |
| ATOM | 7480 | CD2 | PHE | B | 208 | 56.145 | 80.800 | 77.176 | 1.00 | 27.44 |
| ATOM | 7481 | CE1 | PHE | B | 208 | 57.823 | 82.921 | 76.700 | 1.00 | 28.05 |
| ATOM | 7482 | CE2 | PHE | B | 208 | 55.633 | 82.068 | 77.118 | 1.00 | 28.17 |
| ATOM | 7483 | CZ | PHE | B | 208 | 56.482 | 83.138 | 76.888 | 1.00 | 28.67 |
| ATOM | 7484 | N | SER | B | 209 | 59.990 | 76.662 | 76.191 | 1.00 | 26.62 |
| ATOM | 7485 | CA | SER | B | 209 | 60.105 | 75.245 | 76.577 | 1.00 | 26.13 |
| ATOM | 7486 | C | SER | B | 209 | 58.900 | 74.899 | 77.448 | 1.00 | 26.36 |
| ATOM | 7487 | O | SER | B | 209 | 58.979 | 74.171 | 78.431 | 1.00 | 26.32 |
| ATOM | 7488 | CB | SER | B | 209 | 61.416 | 74.957 | 77.306 | 1.00 | 25.55 |
| ATOM | 7489 | OG | SER | B | 209 | 62.530 | 75.099 | 76.429 | 1.00 | 25.11 |
| ATOM | 7490 | N | ALA | B | 210 | 57.767 | 75.434 | 77.053 | 1.00 | 26.03 |
| ATOM | 7491 | CA | ALA | B | 210 | 56.530 | 75.139 | 77.750 | 1.00 | 27.12 |
| ATOM | 7492 | C | ALA | B | 210 | 55.368 | 75.586 | 76.863 | 1.00 | 26.75 |
| ATOM | 7493 | O | ALA | B | 210 | 55.554 | 76.453 | 75.977 | 1.00 | 26.11 |
| ATOM | 7494 | CB | ALA | B | 210 | 56.496 | 75.835 | 79.162 | 1.00 | 26.29 |
| ATOM | 7495 | N | TYR | B | 211 | 54.219 | 74.938 | 77.065 | 1.00 | 26.90 |
| ATOM | 7496 | CA | TYR | B | 211 | 52.957 | 75.218 | 76.366 | 1.00 | 27.97 |
| ATOM | 7497 | C | TYR | B | 211 | 52.230 | 76.354 | 77.033 | 1.00 | 28.34 |
| ATOM | 7498 | O | TYR | B | 211 | 51.469 | 77.130 | 76.423 | 1.00 | 27.52 |
| ATOM | 7499 | CB | TYR | B | 211 | 52.034 | 74.021 | 76.525 | 1.00 | 28.35 |
| ATOM | 7500 | CG | TYR | B | 211 | 50.822 | 74.005 | 75.605 | 1.00 | 28.82 |
| ATOM | 7501 | CD1 | TYR | B | 211 | 50.772 | 74.750 | 74.417 | 1.00 | 28.38 |
| ATOM | 7502 | CD2 | TYR | B | 211 | 49.740 | 73.223 | 75.910 | 1.00 | 28.18 |
| ATOM | 7503 | CE1 | TYR | B | 211 | 49.648 | 74.707 | 73.595 | 1.00 | 25.00 |
| ATOM | 7504 | CE2 | TYR | B | 211 | 48.642 | 73.164 | 75.092 | 1.00 | 26.81 |
| ATOM | 7505 | CZ | TYR | B | 211 | 48.594 | 73.903 | 73.951 | 1.00 | 24.68 |
| ATOM | 7506 | OH | TYR | B | 211 | 47.446 | 73.795 | 73.184 | 1.00 | 27.50 |
| ATOM | 7507 | N | SER | B | 212 | 52.473 | 76.413 | 78.330 | 1.00 | 29.04 |
| ATOM | 7508 | CA | SER | B | 212 | 51.835 | 77.354 | 79.211 | 1.00 | 29.32 |
| ATOM | 7509 | C | SER | B | 212 | 52.259 | 78.790 | 78.947 | 1.00 | 28.36 |
| ATOM | 7510 | O | SER | B | 212 | 53.408 | 79.068 | 78.695 | 1.00 | 28.30 |
| ATOM | 7511 | CB | SER | B | 212 | 52.195 | 76.983 | 80.642 | 1.00 | 29.52 |
| ATOM | 7512 | OG | SER | B | 212 | 51.407 | 77.736 | 81.521 | 1.00 | 34.46 |
| ATOM | 7513 | N | ALA | B | 213 | 51.326 | 79.708 | 79.038 | 1.00 | 28.09 |
| ATOM | 7514 | CA | ALA | B | 213 | 51.693 | 81.106 | 78.991 | 1.00 | 28.66 |
| ATOM | 7515 | C | ALA | B | 213 | 50.814 | 81.878 | 79.964 | 1.00 | 29.25 |
| ATOM | 7516 | O | ALA | B | 213 | 50.257 | 82.912 | 79.640 | 1.00 | 29.81 |
| ATOM | 7517 | CB | ALA | B | 213 | 51.579 | 81.627 | 77.605 | 1.00 | 28.39 |
| ATOM | 7518 | N | LEU | B | 214 | 50.728 | 81.325 | 81.162 | 1.00 | 30.34 |
| ATOM | 7519 | CA | LEU | B | 214 | 49.974 | 81.843 | 82.308 | 1.00 | 30.90 |
| ATOM | 7520 | C | LEU | B | 214 | 50.925 | 81.979 | 83.496 | 1.00 | 30.49 |
| ATOM | 7521 | O | LEU | B | 214 | 51.700 | 81.089 | 83.752 | 1.00 | 29.34 |
| ATOM | 7522 | CB | LEU | B | 214 | 48.948 | 80.788 | 82.727 | 1.00 | 31.14 |
| ATOM | 7523 | CG | LEU | B | 214 | 47.513 | 80.885 | 82.229 | 1.00 | 33.58 |
| ATOM | 7524 | CD1 | LEU | B | 214 | 47.436 | 81.255 | 80.838 | 1.00 | 36.22 |
| ATOM | 7525 | CD2 | LEU | B | 214 | 46.847 | 79.558 | 82.415 | 1.00 | 34.93 |
| ATOM | 7526 | N | TRP | B | 215 | 50.872 | 83.070 | 84.228 | 1.00 | 30.57 |
| ATOM | 7527 | CA | TRP | B | 215 | 51.706 | 83.193 | 85.419 | 1.00 | 30.30 |
| ATOM | 7528 | C | TRP | B | 215 | 50.869 | 83.817 | 86.503 | 1.00 | 30.05 |
| ATOM | 7529 | O | TRP | B | 215 | 50.581 | 84.989 | 86.471 | 1.00 | 30.63 |
| ATOM | 7530 | CB | TRP | B | 215 | 52.962 | 84.052 | 85.179 | 1.00 | 29.99 |
| ATOM | 7531 | CG | TRP | B | 215 | 53.786 | 83.601 | 84.035 | 1.00 | 31.13 |
| ATOM | 7532 | CD1 | TRP | B | 215 | 54.843 | 82.727 | 84.077 | 1.00 | 32.43 |
| ATOM | 7533 | CD2 | TRP | B | 215 | 53.608 | 83.950 | 82.649 | 1.00 | 29.56 |
| ATOM | 7534 | NE1 | TRP | B | 215 | 55.345 | 82.537 | 82.811 | 1.00 | 32.00 |
| ATOM | 7535 | CE2 | TRP | B | 215 | 54.600 | 83.257 | 81.913 | 1.00 | 31.76 |
| ATOM | 7536 | CE3 | TRP | B | 215 | 52.714 | 84.779 | 81.960 | 1.00 | 26.48 |
| ATOM | 7537 | CZ2 | TRP | B | 215 | 54.720 | 83.359 | 80.511 | 1.00 | 32.58 |
| ATOM | 7538 | CZ3 | TRP | B | 215 | 52.810 | 84.873 | 80.562 | 1.00 | 31.02 |
| ATOM | 7539 | CH2 | TRP | B | 215 | 53.820 | 84.172 | 79.854 | 1.00 | 32.58 |
| ATOM | 7540 | N | TRP | B | 216 | 50.484 | 83.038 | 87.478 | 1.00 | 30.12 |
| ATOM | 7541 | CA | TRP | B | 216 | 49.760 | 83.593 | 88.609 | 1.00 | 30.31 |
| ATOM | 7542 | C | TRP | B | 216 | 50.637 | 84.422 | 89.529 | 1.00 | 30.86 |
| ATOM | 7543 | O | TRP | B | 216 | 51.828 | 84.183 | 89.634 | 1.00 | 31.61 |
| ATOM | 7544 | CB | TRP | B | 216 | 49.214 | 82.478 | 89.461 | 1.00 | 29.58 |
| ATOM | 7545 | CG | TRP | B | 216 | 48.015 | 81.767 | 88.979 | 1.00 | 28.91 |
| ATOM | 7546 | CD1 | TRP | B | 216 | 47.986 | 80.546 | 88.384 | 1.00 | 29.29 |
| ATOM | 7547 | CD2 | TRP | B | 216 | 46.646 | 82.147 | 89.184 | 1.00 | 28.16 |
| ATOM | 7548 | NE1 | TRP | B | 216 | 46.688 | 80.168 | 88.161 | 1.00 | 31.36 |
| ATOM | 7549 | CE2 | TRP | B | 216 | 45.845 | 81.133 | 88.638 | 1.00 | 28.27 |
| ATOM | 7550 | CE3 | TRP | B | 216 | 46.016 | 83.268 | 89.742 | 1.00 | 29.24 |
| ATOM | 7551 | CZ2 | TRP | B | 216 | 44.457 | 81.193 | 88.627 | 1.00 | 30.27 |
| ATOM | 7552 | CZ3 | TRP | B | 216 | 44.628 | 83.340 | 89.726 | 1.00 | 31.04 |
| ATOM | 7553 | CH2 | TRP | B | 216 | 43.853 | 82.293 | 89.191 | 1.00 | 29.17 |
| ATOM | 7554 | N | SER | B | 217 | 50.037 | 85.441 | 90.143 | 1.00 | 31.96 |
| ATOM | 7555 | CA | SER | B | 217 | 50.629 | 86.205 | 91.257 | 1.00 | 32.49 |
| ATOM | 7556 | C | SER | B | 217 | 50.974 | 85.256 | 92.383 | 1.00 | 32.33 |
| ATOM | 7557 | O | SER | B | 217 | 50.307 | 84.243 | 92.544 | 1.00 | 30.55 |
| ATOM | 7558 | CB | SER | B | 217 | 49.532 | 87.020 | 91.937 | 1.00 | 32.34 |
| ATOM | 7559 | OG | SER | B | 217 | 49.459 | 88.290 | 91.440 | 1.00 | 34.18 |
| ATOM | 7560 | N | PRO | B | 218 | 51.911 | 85.632 | 93.244 | 1.00 | 33.78 |
| ATOM | 7561 | CA | PRO | B | 218 | 52.202 | 84.814 | 94.428 | 1.00 | 34.88 |
| ATOM | 7562 | C | PRO | B | 218 | 51.001 | 84.778 | 95.355 | 1.00 | 36.39 |
| ATOM | 7563 | O | PRO | B | 218 | 50.806 | 83.758 | 95.986 | 1.00 | 37.96 |
| ATOM | 7564 | CB | PRO | B | 218 | 53.396 | 85.525 | 95.063 | 1.00 | 35.69 |
| ATOM | 7565 | CG | PRO | B | 218 | 54.005 | 86.295 | 93.922 | 1.00 | 35.14 |
| ATOM | 7566 | CD | PRO | B | 218 | 52.783 | 86.806 | 93.157 | 1.00 | 33.44 |
| ATOM | 7567 | N | ASN | B | 219 | 50.232 | 85.859 | 95.397 | 1.00 | 37.49 |
| ATOM | 7568 | CA | ASN | B | 219 | 48.946 | 85.987 | 96.108 | 1.00 | 39.26 |
| ATOM | 7569 | C | ASN | B | 219 | 47.896 | 85.043 | 95.565 | 1.00 | 39.67 |
| ATOM | 7570 | O | ASN | B | 219 | 47.089 | 84.442 | 96.291 | 1.00 | 39.72 |
| ATOM | 7571 | CB | ASN | B | 219 | 48.330 | 87.410 | 95.850 | 1.00 | 38.98 |
| ATOM | 7572 | CG | ASN | B | 219 | 48.433 | 88.345 | 97.060 | 1.00 | 41.91 |
| ATOM | 7573 | OD1 | ASN | B | 219 | 48.311 | 87.888 | 98.183 | 1.00 | 46.79 |
| ATOM | 7574 | ND2 | ASN | B | 219 | 48.625 | 89.664 | 96.831 | 1.00 | 40.99 |
| ATOM | 7575 | N | GLY | B | 220 | 47.865 | 84.980 | 94.246 | 1.00 | 39.21 |
| ATOM | 7576 | CA | GLY | B | 220 | 46.757 | 84.360 | 93.561 | 1.00 | 38.69 |
| ATOM | 7577 | C | GLY | B | 220 | 45.819 | 85.466 | 93.098 | 1.00 | 38.39 |
| ATOM | 7578 | O | GLY | B | 220 | 44.786 | 85.208 | 92.488 | 1.00 | 38.87 |
| ATOM | 7579 | N | THR | B | 221 | 46.198 | 86.709 | 93.358 | 1.00 | 37.66 |
| ATOM | 7580 | CA | THR | B | 221 | 45.350 | 87.810 | 93.017 | 1.00 | 37.51 |
| ATOM | 7581 | C | THR | B | 221 | 45.312 | 88.018 | 91.540 | 1.00 | 37.18 |
| ATOM | 7582 | O | THR | B | 221 | 44.240 | 88.020 | 90.946 | 1.00 | 36.90 |
| ATOM | 7583 | CB | THR | B | 221 | 45.837 | 89.097 | 93.696 | 1.00 | 37.70 |
| ATOM | 7584 | OG1 | THR | B | 221 | 45.452 | 89.073 | 95.067 | 1.00 | 39.62 |
| ATOM | 7585 | CG2 | THR | B | 221 | 45.083 | 90.325 | 93.187 | 1.00 | 37.99 |
| ATOM | 7586 | N | PHE | B | 222 | 46.495 | 88.197 | 90.954 | 1.00 | 36.74 |
| ATOM | 7587 | CA | PHE | B | 222 | 46.612 | 88.519 | 89.558 | 1.00 | 36.26 |
| ATOM | 7588 | C | PHE | B | 222 | 46.906 | 87.321 | 88.696 | 1.00 | 36.17 |
| ATOM | 7589 | O | PHE | B | 222 | 47.702 | 86.467 | 89.069 | 1.00 | 36.16 |
| ATOM | 7590 | CB | PHE | B | 222 | 47.740 | 89.503 | 89.318 | 1.00 | 35.78 |
| ATOM | 7591 | CG | PHE | B | 222 | 47.521 | 90.848 | 89.914 | 1.00 | 37.68 |
| ATOM | 7592 | CD1 | PHE | B | 222 | 46.630 | 91.752 | 89.335 | 1.00 | 37.31 |
| ATOM | 7593 | CD2 | PHE | B | 222 | 48.253 | 91.252 | 91.030 | 1.00 | 37.75 |
| ATOM | 7594 | CE1 | PHE | B | 222 | 46.446 | 93.032 | 89.887 | 1.00 | 37.85 |
| ATOM | 7595 | CE2 | PHE | B | 222 | 48.072 | 92.530 | 91.576 | 1.00 | 38.13 |
| ATOM | 7596 | CZ | PHE | B | 222 | 47.179 | 93.415 | 91.009 | 1.00 | 37.06 |
| ATOM | 7597 | N | LEU | B | 223 | 46.328 | 87.331 | 87.488 | 1.00 | 35.02 |
| ATOM | 7598 | CA | LEU | B | 223 | 46.599 | 86.308 | 86.501 | 1.00 | 35.02 |
| ATOM | 7599 | C | LEU | B | 223 | 47.239 | 87.009 | 85.291 | 1.00 | 33.64 |
| ATOM | 7600 | O | LEU | B | 223 | 46.591 | 87.766 | 84.585 | 1.00 | 34.11 |
| ATOM | 7601 | CB | LEU | B | 223 | 45.301 | 85.598 | 86.101 | 1.00 | 34.43 |
| ATOM | 7602 | CG | LEU | B | 223 | 45.364 | 84.124 | 85.650 | 1.00 | 37.73 |
| ATOM | 7603 | CD1 | LEU | B | 223 | 44.380 | 83.825 | 84.519 | 1.00 | 35.89 |
| ATOM | 7604 | CD2 | LEU | B | 223 | 46.763 | 83.670 | 85.228 | 1.00 | 37.28 |
| ATOM | 7605 | N | ALA | B | 224 | 48.524 | 86.815 | 85.068 | 1.00 | 32.28 |
| ATOM | 7606 | CA | ALA | B | 224 | 49.121 | 87.381 | 83.858 | 1.00 | 31.54 |
| ATOM | 7607 | C | ALA | B | 224 | 49.196 | 86.324 | 82.780 | 1.00 | 30.26 |
| ATOM | 7608 | O | ALA | B | 224 | 49.320 | 85.158 | 83.085 | 1.00 | 28.46 |
| ATOM | 7609 | CB | ALA | B | 224 | 50.481 | 87.937 | 84.122 | 1.00 | 31.38 |
| ATOM | 7610 | N | TYR | B | 225 | 49.154 | 86.766 | 81.522 | 1.00 | 30.30 |
| ATOM | 7611 | CA | TYR | B | 225 | 49.173 | 85.882 | 80.367 | 1.00 | 29.65 |
| ATOM | 7612 | C | TYR | B | 225 | 49.641 | 86.597 | 79.120 | 1.00 | 29.29 |
| ATOM | 7613 | O | TYR | B | 225 | 49.570 | 87.822 | 78.998 | 1.00 | 29.09 |
| ATOM | 7614 | CB | TYR | B | 225 | 47.801 | 85.269 | 80.059 | 1.00 | 29.11 |
| ATOM | 7615 | CG | TYR | B | 225 | 46.745 | 86.248 | 79.586 | 1.00 | 30.78 |
| ATOM | 7616 | CD1 | TYR | B | 225 | 45.958 | 86.910 | 80.487 | 1.00 | 32.08 |
| ATOM | 7617 | CD2 | TYR | B | 225 | 46.512 | 86.490 | 78.230 | 1.00 | 30.84 |
| ATOM | 7618 | CE1 | TYR | B | 225 | 44.970 | 87.785 | 80.089 | 1.00 | 31.25 |
| ATOM | 7619 | CE2 | TYR | B | 225 | 45.530 | 87.377 | 77.835 | 1.00 | 30.95 |
| ATOM | 7620 | CZ | TYR | B | 225 | 44.767 | 88.025 | 78.777 | 1.00 | 32.45 |
| ATOM | 7621 | OH | TYR | B | 225 | 43.766 | 88.926 | 78.436 | 1.00 | 34.02 |
| ATOM | 7622 | N | ALA | B | 226 | 50.127 | 85.782 | 78.196 | 1.00 | 28.63 |
| ATOM | 7623 | CA | ALA | B | 226 | 50.610 | 86.237 | 76.907 | 1.00 | 28.11 |
| ATOM | 7624 | C | ALA | B | 226 | 49.622 | 85.793 | 75.854 | 1.00 | 27.59 |
| ATOM | 7625 | O | ALA | B | 226 | 48.916 | 84.793 | 76.027 | 1.00 | 27.17 |
| ATOM | 7626 | CB | ALA | B | 226 | 51.983 | 85.621 | 76.608 | 1.00 | 27.99 |
| ATOM | 7627 | N | GLN | B | 227 | 49.595 | 86.533 | 74.761 | 1.00 | 27.13 |
| ATOM | 7628 | CA | GLN | B | 227 | 48.750 | 86.216 | 73.620 | 1.00 | 27.83 |
| ATOM | 7629 | C | GLN | B | 227 | 49.612 | 86.226 | 72.383 | 1.00 | 27.32 |
| ATOM | 7630 | O | GLN | B | 227 | 50.416 | 87.141 | 72.181 | 1.00 | 25.90 |
| ATOM | 7631 | CB | GLN | B | 227 | 47.664 | 87.257 | 73.452 | 1.00 | 28.61 |
| ATOM | 7632 | CG | GLN | B | 227 | 46.691 | 86.956 | 72.370 | 1.00 | 27.98 |
| ATOM | 7633 | CD | GLN | B | 227 | 45.812 | 88.148 | 72.094 | 1.00 | 29.34 |
| ATOM | 7634 | OE1 | GLN | B | 227 | 46.259 | 89.131 | 71.487 | 1.00 | 29.32 |
| ATOM | 7635 | NE2 | GLN | B | 227 | 44.579 | 88.086 | 72.559 | 1.00 | 25.03 |
| ATOM | 7636 | N | PHE | B | 228 | 49.498 | 85.179 | 71.583 | 1.00 | 26.63 |
| ATOM | 7637 | CA | PHE | B | 228 | 50.315 | 85.107 | 70.405 | 1.00 | 27.20 |
| ATOM | 7638 | C | PHE | B | 228 | 49.429 | 85.135 | 69.209 | 1.00 | 26.71 |
| ATOM | 7639 | O | PHE | B | 228 | 48.351 | 84.548 | 69.235 | 1.00 | 28.26 |
| ATOM | 7640 | CB | PHE | B | 228 | 51.194 | 83.894 | 70.415 | 1.00 | 26.80 |
| ATOM | 7641 | CG | PHE | B | 228 | 51.989 | 83.746 | 71.681 | 1.00 | 27.53 |
| ATOM | 7642 | CD1 | PHE | B | 228 | 53.196 | 84.397 | 71.831 | 1.00 | 25.12 |
| ATOM | 7643 | CD2 | PHE | B | 228 | 51.517 | 82.964 | 72.729 | 1.00 | 24.95 |
| ATOM | 7644 | CE1 | PHE | B | 228 | 53.927 | 84.259 | 72.979 | 1.00 | 23.41 |
| ATOM | 7645 | CE2 | PHE | B | 228 | 52.264 | 82.821 | 73.888 | 1.00 | 24.50 |
| ATOM | 7646 | CZ | PHE | B | 228 | 53.472 | 83.460 | 74.006 | 1.00 | 22.75 |
| ATOM | 7647 | N | ASN | B | 229 | 49.865 | 85.851 | 68.186 | 1.00 | 26.75 |
| ATOM | 7648 | CA | ASN | B | 229 | 49.084 | 85.996 | 66.988 | 1.00 | 28.12 |
| ATOM | 7649 | C | ASN | B | 229 | 49.925 | 85.496 | 65.821 | 1.00 | 28.58 |
| ATOM | 7650 | O | ASN | B | 229 | 50.984 | 86.052 | 65.514 | 1.00 | 28.66 |
| ATOM | 7651 | CB | ASN | B | 229 | 48.654 | 87.465 | 66.821 | 1.00 | 28.28 |
| ATOM | 7652 | CG | ASN | B | 229 | 47.711 | 87.671 | 65.636 | 1.00 | 28.87 |
| ATOM | 7653 | OD1 | ASN | B | 229 | 47.694 | 86.887 | 64.668 | 1.00 | 27.69 |
| ATOM | 7654 | ND2 | ASN | B | 229 | 46.909 | 88.711 | 65.731 | 1.00 | 32.77 |
| ATOM | 7655 | N | ASP | B | 230 | 49.438 | 84.422 | 65.198 | 1.00 | 29.23 |
| ATOM | 7656 | CA | ASP | B | 230 | 50.127 | 83.693 | 64.139 | 1.00 | 29.42 |
| ATOM | 7657 | C | ASP | B | 230 | 49.504 | 83.839 | 62.730 | 1.00 | 29.87 |
| ATOM | 7658 | O | ASP | B | 230 | 49.922 | 83.182 | 61.765 | 1.00 | 28.49 |
| ATOM | 7659 | CB | ASP | B | 230 | 50.094 | 82.215 | 64.530 | 1.00 | 29.90 |
| ATOM | 7660 | CG | ASP | B | 230 | 51.209 | 81.847 | 65.465 | 1.00 | 30.77 |
| ATOM | 7661 | OD1 | ASP | B | 230 | 51.273 | 82.457 | 66.540 | 1.00 | 36.25 |
| ATOM | 7662 | OD2 | ASP | B | 230 | 52.063 | 80.994 | 65.214 | 1.00 | 31.62 |
| ATOM | 7663 | N | THR | B | 231 | 48.533 | 84.724 | 62.620 | 1.00 | 30.50 |
| ATOM | 7664 | CA | THR | B | 231 | 47.829 | 84.952 | 61.365 | 1.00 | 31.25 |
| ATOM | 7665 | C | THR | B | 231 | 48.641 | 84.879 | 60.118 | 1.00 | 31.98 |
| ATOM | 7666 | O | THR | B | 231 | 48.215 | 84.207 | 59.184 | 1.00 | 33.25 |
| ATOM | 7667 | CB | THR | B | 231 | 47.149 | 86.272 | 61.366 | 1.00 | 31.46 |
| ATOM | 7668 | OG1 | THR | B | 231 | 46.132 | 86.231 | 62.345 | 1.00 | 31.85 |
| ATOM | 7669 | CG2 | THR | B | 231 | 46.333 | 86.506 | 60.005 | 1.00 | 34.71 |
| ATOM | 7670 | N | GLU | B | 232 | 49.772 | 85.569 | 60.046 | 1.00 | 31.14 |
| ATOM | 7671 | CA | GLU | B | 232 | 50.509 | 85.533 | 58.801 | 1.00 | 31.74 |
| ATOM | 7672 | C | GLU | B | 232 | 51.747 | 84.650 | 58.894 | 1.00 | 30.99 |
| ATOM | 7673 | O | GLU | B | 232 | 52.658 | 84.822 | 58.132 | 1.00 | 30.42 |
| ATOM | 7674 | CB | GLU | B | 232 | 50.931 | 86.941 | 58.413 | 1.00 | 33.19 |
| ATOM | 7675 | CG | GLU | B | 232 | 49.805 | 87.952 | 58.494 | 1.00 | 36.69 |
| ATOM | 7676 | CD | GLU | B | 232 | 50.150 | 89.268 | 57.821 | 1.00 | 43.55 |
| ATOM | 7677 | OE1 | GLU | B | 232 | 50.032 | 89.349 | 56.567 | 1.00 | 45.34 |
| ATOM | 7678 | OE2 | GLU | B | 232 | 50.526 | 90.218 | 58.561 | 1.00 | 48.36 |
| ATOM | 7679 | N | VAL | B | 233 | 51.805 | 83.752 | 59.872 | 1.00 | 29.36 |
| ATOM | 7680 | CA | VAL | B | 233 | 52.922 | 82.852 | 59.945 | 1.00 | 28.97 |
| ATOM | 7681 | C | VAL | B | 233 | 52.656 | 81.746 | 58.927 | 1.00 | 27.82 |
| ATOM | 7682 | O | VAL | B | 233 | 51.558 | 81.243 | 58.860 | 1.00 | 27.90 |
| ATOM | 7683 | CB | VAL | B | 233 | 53.056 | 82.322 | 61.344 | 1.00 | 29.57 |
| ATOM | 7684 | CG1 | VAL | B | 233 | 54.181 | 81.310 | 61.438 | 1.00 | 29.42 |
| ATOM | 7685 | CG2 | VAL | B | 233 | 53.265 | 83.519 | 62.340 | 1.00 | 31.02 |
| ATOM | 7686 | N | PRO | B | 234 | 53.611 | 81.429 | 58.066 | 1.00 | 27.73 |
| ATOM | 7687 | CA | PRO | B | 234 | 53.378 | 80.375 | 57.063 | 1.00 | 28.03 |
| ATOM | 7688 | C | PRO | B | 234 | 53.297 | 78.995 | 57.693 | 1.00 | 28.08 |
| ATOM | 7689 | O | PRO | B | 234 | 53.815 | 78.743 | 58.790 | 1.00 | 29.28 |
| ATOM | 7690 | CB | PRO | B | 234 | 54.580 | 80.487 | 56.115 | 1.00 | 28.06 |
| ATOM | 7691 | CG | PRO | B | 234 | 55.243 | 81.799 | 56.477 | 1.00 | 28.38 |
| ATOM | 7692 | CD | PRO | B | 234 | 54.928 | 82.072 | 57.908 | 1.00 | 27.42 |
| ATOM | 7693 | N | LEU | B | 235 | 52.607 | 78.107 | 57.008 | 1.00 | 27.75 |
| ATOM | 7694 | CA | LEU | B | 235 | 52.366 | 76.786 | 57.502 | 1.00 | 27.64 |
| ATOM | 7695 | C | LEU | B | 235 | 53.280 | 75.745 | 56.922 | 1.00 | 27.09 |
| ATOM | 7696 | O | LEU | B | 235 | 53.567 | 75.789 | 55.734 | 1.00 | 26.39 |
| ATOM | 7697 | CB | LEU | B | 235 | 50.918 | 76.398 | 57.165 | 1.00 | 29.04 |
| ATOM | 7698 | CG | LEU | B | 235 | 49.888 | 77.425 | 57.648 | 1.00 | 30.71 |
| ATOM | 7699 | CD1 | LEU | B | 235 | 48.532 | 77.339 | 56.918 | 1.00 | 34.01 |
| ATOM | 7700 | CD2 | LEU | B | 235 | 49.669 | 77.245 | 59.095 | 1.00 | 30.08 |
| ATOM | 7701 | N | ILE | B | 236 | 53.806 | 74.853 | 57.781 | 1.00 | 25.72 |
| ATOM | 7702 | CA | ILE | B | 236 | 54.442 | 73.660 | 57.283 | 1.00 | 24.98 |
| ATOM | 7703 | C | ILE | B | 236 | 53.316 | 72.667 | 57.124 | 1.00 | 24.14 |
| ATOM | 7704 | O | ILE | B | 236 | 52.377 | 72.597 | 57.938 | 1.00 | 23.94 |
| ATOM | 7705 | CB | ILE | B | 236 | 55.598 | 73.143 | 58.182 | 1.00 | 25.42 |
| ATOM | 7706 | CG1 | ILE | B | 236 | 56.360 | 72.041 | 57.439 | 1.00 | 28.92 |
| ATOM | 7707 | CG2 | ILE | B | 236 | 55.122 | 72.770 | 59.579 | 1.00 | 26.00 |
| ATOM | 7708 | CD1 | ILE | B | 236 | 56.567 | 70.833 | 58.283 | 1.00 | 33.95 |
| ATOM | 7709 | N | GLU | B | 237 | 53.410 | 71.894 | 56.069 | 1.00 | 23.99 |
| ATOM | 7710 | CA | GLU | B | 237 | 52.372 | 70.939 | 55.679 | 1.00 | 24.82 |
| ATOM | 7711 | C | GLU | B | 237 | 53.073 | 69.617 | 55.389 | 1.00 | 24.01 |
| ATOM | 7712 | O | GLU | B | 237 | 54.087 | 69.604 | 54.769 | 1.00 | 23.08 |
| ATOM | 7713 | CB | GLU | B | 237 | 51.611 | 71.483 | 54.459 | 1.00 | 24.84 |
| ATOM | 7714 | CG | GLU | B | 237 | 50.961 | 72.848 | 54.752 | 1.00 | 27.02 |
| ATOM | 7715 | CD | GLU | B | 237 | 49.817 | 73.264 | 53.816 | 1.00 | 30.40 |
| ATOM | 7716 | OE1 | GLU | B | 237 | 49.655 | 72.689 | 52.733 | 1.00 | 30.28 |
| ATOM | 7717 | OE2 | GLU | B | 237 | 49.055 | 74.198 | 54.167 | 1.00 | 33.11 |
| ATOM | 7718 | N | TYR | B | 238 | 52.601 | 68.533 | 55.978 | 1.00 | 23.87 |
| ATOM | 7719 | CA | TYR | B | 238 | 53.143 | 67.213 | 55.721 | 1.00 | 23.57 |
| ATOM | 7720 | C | TYR | B | 238 | 52.022 | 66.237 | 55.926 | 1.00 | 24.22 |
| ATOM | 7721 | O | TYR | B | 238 | 51.055 | 66.512 | 56.644 | 1.00 | 22.25 |
| ATOM | 7722 | CB | TYR | B | 238 | 54.342 | 66.855 | 56.632 | 1.00 | 23.29 |
| ATOM | 7723 | CG | TYR | B | 238 | 54.099 | 67.003 | 58.135 | 1.00 | 24.82 |
| ATOM | 7724 | CD1 | TYR | B | 238 | 54.357 | 68.200 | 58.773 | 1.00 | 24.60 |
| ATOM | 7725 | CD2 | TYR | B | 238 | 53.622 | 65.964 | 58.897 | 1.00 | 25.53 |
| ATOM | 7726 | CE1 | TYR | B | 238 | 54.136 | 68.368 | 60.097 | 1.00 | 25.40 |
| ATOM | 7727 | CE2 | TYR | B | 238 | 53.413 | 66.126 | 60.293 | 1.00 | 24.43 |
| ATOM | 7728 | CZ | TYR | B | 238 | 53.694 | 67.340 | 60.857 | 1.00 | 24.49 |
| ATOM | 7729 | OH | TYR | B | 238 | 53.549 | 67.600 | 62.182 | 1.00 | 25.79 |
| ATOM | 7730 | N | SER | B | 239 | 52.164 | 65.090 | 55.265 | 1.00 | 25.22 |
| ATOM | 7731 | CA | SER | B | 239 | 51.223 | 63.983 | 55.363 | 1.00 | 25.50 |
| ATOM | 7732 | C | SER | B | 239 | 51.349 | 63.189 | 56.649 | 1.00 | 25.41 |
| ATOM | 7733 | O | SER | B | 239 | 52.422 | 63.075 | 57.174 | 1.00 | 26.57 |
| ATOM | 7734 | CB | SER | B | 239 | 51.485 | 63.044 | 54.211 | 1.00 | 25.24 |
| ATOM | 7735 | OG | SER | B | 239 | 51.186 | 63.705 | 53.021 | 1.00 | 24.84 |
| ATOM | 7736 | N | PHE | B | 240 | 50.220 | 62.717 | 57.179 | 1.00 | 25.98 |
| ATOM | 7737 | CA | PHE | B | 240 | 50.158 | 61.814 | 58.320 | 1.00 | 25.45 |
| ATOM | 7738 | C | PHE | B | 240 | 49.294 | 60.655 | 57.830 | 1.00 | 25.82 |
| ATOM | 7739 | O | PHE | B | 240 | 48.155 | 60.873 | 57.414 | 1.00 | 25.70 |
| ATOM | 7740 | CB | PHE | B | 240 | 49.484 | 62.466 | 59.537 | 1.00 | 25.55 |
| ATOM | 7741 | CG | PHE | B | 240 | 49.625 | 61.658 | 60.781 | 1.00 | 25.99 |
| ATOM | 7742 | CD1 | PHE | B | 240 | 50.773 | 61.749 | 61.550 | 1.00 | 28.27 |
| ATOM | 7743 | CD2 | PHE | B | 240 | 48.679 | 60.743 | 61.130 | 1.00 | 26.52 |
| ATOM | 7744 | CE1 | PHE | B | 240 | 50.940 | 60.961 | 62.696 | 1.00 | 29.81 |
| ATOM | 7745 | CE2 | PHE | B | 240 | 48.852 | 59.949 | 62.251 | 1.00 | 28.54 |
| ATOM | 7746 | CZ | PHE | B | 240 | 49.990 | 60.060 | 63.027 | 1.00 | 29.91 |
| ATOM | 7747 | N | TYR | B | 241 | 49.820 | 59.437 | 57.868 | 1.00 | 25.66 |
| ATOM | 7748 | CA | TYR | B | 241 | 49.142 | 58.310 | 57.264 | 1.00 | 25.52 |
| ATOM | 7749 | C | TYR | B | 241 | 48.157 | 57.530 | 58.182 | 1.00 | 26.95 |
| ATOM | 7750 | O | TYR | B | 241 | 47.129 | 57.045 | 57.706 | 1.00 | 25.02 |
| ATOM | 7751 | CB | TYR | B | 241 | 50.208 | 57.419 | 56.620 | 1.00 | 25.51 |
| ATOM | 7752 | CG | TYR | B | 241 | 51.079 | 58.205 | 55.630 | 1.00 | 25.02 |
| ATOM | 7753 | CD1 | TYR | B | 241 | 50.642 | 58.430 | 54.333 | 1.00 | 24.85 |
| ATOM | 7754 | CD2 | TYR | B | 241 | 52.313 | 58.742 | 56.019 | 1.00 | 24.74 |
| ATOM | 7755 | CE1 | TYR | B | 241 | 51.385 | 59.183 | 53.434 | 1.00 | 24.84 |
| ATOM | 7756 | CE2 | TYR | B | 241 | 53.084 | 59.469 | 55.143 | 1.00 | 25.22 |
| ATOM | 7757 | CZ | TYR | B | 241 | 52.599 | 59.681 | 53.841 | 1.00 | 24.72 |
| ATOM | 7758 | OH | TYR | B | 241 | 53.307 | 60.384 | 52.982 | 1.00 | 25.39 |
| ATOM | 7759 | N | SER | B | 242 | 48.442 | 57.507 | 59.498 | 1.00 | 28.06 |
| ATOM | 7760 | CA | SER | B | 242 | 47.718 | 56.743 | 60.527 | 1.00 | 28.10 |
| ATOM | 7761 | C | SER | B | 242 | 47.597 | 55.270 | 60.275 | 1.00 | 27.38 |
| ATOM | 7762 | O | SER | B | 242 | 48.408 | 54.696 | 59.581 | 1.00 | 27.05 |
| ATOM | 7763 | CB | SER | B | 242 | 46.339 | 57.314 | 60.912 | 1.00 | 28.67 |
| ATOM | 7764 | OG | SER | B | 242 | 45.832 | 58.068 | 59.883 | 1.00 | 34.27 |
| ATOM | 7765 | N | ASP | B | 243 | 46.621 | 54.657 | 60.943 | 1.00 | 27.89 |
| ATOM | 7766 | CA | ASP | B | 243 | 46.342 | 53.244 | 60.845 | 1.00 | 29.32 |
| ATOM | 7767 | C | ASP | B | 243 | 46.001 | 52.909 | 59.380 | 1.00 | 28.89 |
| ATOM | 7768 | O | ASP | B | 243 | 45.590 | 53.790 | 58.607 | 1.00 | 29.54 |
| ATOM | 7769 | CB | ASP | B | 243 | 45.185 | 52.876 | 61.818 | 1.00 | 30.16 |
| ATOM | 7770 | CG | ASP | B | 243 | 45.456 | 53.350 | 63.300 | 1.00 | 35.73 |
| ATOM | 7771 | OD1 | ASP | B | 243 | 46.600 | 53.169 | 63.810 | 1.00 | 38.58 |
| ATOM | 7772 | OD2 | ASP | B | 243 | 44.608 | 53.961 | 64.025 | 1.00 | 39.36 |
| ATOM | 7773 | N | GLU | B | 244 | 46.212 | 51.669 | 58.987 | 1.00 | 28.75 |
| ATOM | 7774 | CA | GLU | B | 244 | 45.862 | 51.205 | 57.648 | 1.00 | 30.00 |
| ATOM | 7775 | C | GLU | B | 244 | 44.417 | 51.504 | 57.191 | 1.00 | 29.87 |
| ATOM | 7776 | O | GLU | B | 244 | 44.161 | 51.540 | 55.985 | 1.00 | 29.78 |
| ATOM | 7777 | CB | GLU | B | 244 | 45.847 | 49.685 | 57.590 | 1.00 | 31.02 |
| ATOM | 7778 | CG | GLU | B | 244 | 47.091 | 48.927 | 57.831 | 1.00 | 33.19 |
| ATOM | 7779 | CD | GLU | B | 244 | 46.840 | 47.448 | 57.551 | 1.00 | 37.41 |
| ATOM | 7780 | OE1 | GLU | B | 244 | 46.089 | 47.131 | 56.597 | 1.00 | 35.31 |
| ATOM | 7781 | OE2 | GLU | B | 244 | 47.418 | 46.600 | 58.279 | 1.00 | 43.04 |
| ATOM | 7782 | N | SER | B | 245 | 43.478 | 51.638 | 58.136 | 1.00 | 28.42 |
| ATOM | 7783 | CA | SER | B | 245 | 42.078 | 51.855 | 57.793 | 1.00 | 28.88 |
| ATOM | 7784 | C | SER | B | 245 | 41.792 | 53.213 | 57.185 | 1.00 | 27.78 |
| ATOM | 7785 | O | SER | B | 245 | 40.774 | 53.415 | 56.553 | 1.00 | 29.01 |
| ATOM | 7786 | CB | SER | B | 245 | 41.185 | 51.664 | 59.043 | 1.00 | 28.82 |
| ATOM | 7787 | OG | SER | B | 245 | 41.671 | 52.500 | 60.086 | 1.00 | 30.11 |
| ATOM | 7788 | N | LEU | B | 246 | 42.658 | 54.173 | 57.389 | 1.00 | 26.88 |
| ATOM | 7789 | CA | LEU | B | 246 | 42.419 | 55.476 | 56.821 | 1.00 | 27.33 |
| ATOM | 7790 | C | LEU | B | 246 | 42.697 | 55.448 | 55.306 | 1.00 | 26.18 |
| ATOM | 7791 | O | LEU | B | 246 | 43.813 | 55.191 | 54.876 | 1.00 | 25.08 |
| ATOM | 7792 | CB | LEU | B | 246 | 43.354 | 56.483 | 57.425 | 1.00 | 27.45 |
| ATOM | 7793 | CG | LEU | B | 246 | 42.894 | 57.878 | 57.761 | 1.00 | 31.22 |
| ATOM | 7794 | CD1 | LEU | B | 246 | 43.988 | 58.847 | 57.373 | 1.00 | 32.53 |
| ATOM | 7795 | CD2 | LEU | B | 246 | 41.541 | 58.313 | 57.252 | 1.00 | 31.50 |
| ATOM | 7796 | N | GLN | B | 247 | 41.701 | 55.804 | 54.528 | 1.00 | 25.48 |
| ATOM | 7797 | CA | GLN | B | 247 | 41.773 | 55.696 | 53.090 | 1.00 | 25.50 |
| ATOM | 7798 | C | GLN | B | 247 | 42.559 | 56.842 | 52.452 | 1.00 | 25.73 |
| ATOM | 7799 | O | GLN | B | 247 | 43.349 | 56.623 | 51.553 | 1.00 | 26.11 |
| ATOM | 7800 | CB | GLN | B | 247 | 40.339 | 55.580 | 52.521 | 1.00 | 24.84 |
| ATOM | 7801 | CG | GLN | B | 247 | 40.311 | 55.160 | 51.065 | 1.00 | 25.22 |
| ATOM | 7802 | CD | GLN | B | 247 | 38.897 | 54.968 | 50.524 | 1.00 | 25.36 |
| ATOM | 7803 | OE1 | GLN | B | 247 | 37.963 | 55.646 | 50.962 | 1.00 | 24.22 |
| ATOM | 7804 | NE2 | GLN | B | 247 | 38.741 | 54.041 | 49.583 | 1.00 | 21.11 |
| ATOM | 7805 | N | TYR | B | 248 | 42.307 | 58.063 | 52.886 | 1.00 | 26.15 |
| ATOM | 7806 | CA | TYR | B | 248 | 43.064 | 59.198 | 52.417 | 1.00 | 26.69 |
| ATOM | 7807 | C | TYR | B | 248 | 43.936 | 59.730 | 53.539 | 1.00 | 27.73 |
| ATOM | 7808 | O | TYR | B | 248 | 43.465 | 59.967 | 54.649 | 1.00 | 27.01 |
| ATOM | 7809 | CB | TYR | B | 248 | 42.149 | 60.327 | 51.953 | 1.00 | 26.34 |
| ATOM | 7810 | CG | TYR | B | 248 | 41.422 | 60.005 | 50.663 | 1.00 | 27.01 |
| ATOM | 7811 | CD1 | TYR | B | 248 | 40.307 | 59.173 | 50.666 | 1.00 | 26.91 |
| ATOM | 7812 | CD2 | TYR | B | 248 | 41.840 | 60.546 | 49.441 | 1.00 | 27.29 |
| ATOM | 7813 | CE1 | TYR | B | 248 | 39.604 | 58.896 | 49.493 | 1.00 | 27.29 |
| ATOM | 7814 | CE2 | TYR | B | 248 | 41.154 | 60.242 | 48.248 | 1.00 | 28.74 |
| ATOM | 7815 | CZ | TYR | B | 248 | 40.044 | 59.408 | 48.291 | 1.00 | 27.43 |
| ATOM | 7816 | OH | TYR | B | 248 | 39.357 | 59.124 | 47.142 | 1.00 | 24.10 |
| ATOM | 7817 | N | PRO | B | 249 | 45.213 | 59.930 | 53.242 | 1.00 | 28.25 |
| ATOM | 7818 | CA | PRO | B | 249 | 46.134 | 60.497 | 54.223 | 1.00 | 28.79 |
| ATOM | 7819 | C | PRO | B | 249 | 45.698 | 61.885 | 54.691 | 1.00 | 29.17 |
| ATOM | 7820 | O | PRO | B | 249 | 45.064 | 62.633 | 53.951 | 1.00 | 26.49 |
| ATOM | 7821 | CB | PRO | B | 249 | 47.462 | 60.574 | 53.462 | 1.00 | 28.57 |
| ATOM | 7822 | CG | PRO | B | 249 | 47.306 | 59.622 | 52.325 | 1.00 | 29.61 |
| ATOM | 7823 | CD | PRO | B | 249 | 45.876 | 59.583 | 51.984 | 1.00 | 27.88 |
| ATOM | 7824 | N | LYS | B | 250 | 45.985 | 62.191 | 55.961 | 1.00 | 29.80 |
| ATOM | 7825 | CA | LYS | B | 250 | 45.693 | 63.513 | 56.475 | 1.00 | 29.97 |
| ATOM | 7826 | C | LYS | B | 250 | 46.885 | 64.417 | 56.200 | 1.00 | 29.32 |
| ATOM | 7827 | O | LYS | B | 250 | 48.024 | 63.962 | 56.092 | 1.00 | 28.82 |
| ATOM | 7828 | CB | LYS | B | 250 | 45.389 | 63.469 | 57.988 | 1.00 | 31.46 |
| ATOM | 7829 | CG | LYS | B | 250 | 44.694 | 64.750 | 58.497 | 1.00 | 35.67 |
| ATOM | 7830 | CD | LYS | B | 250 | 44.889 | 65.052 | 60.033 | 1.00 | 41.42 |
| ATOM | 7831 | CE | LYS | B | 250 | 45.094 | 66.583 | 60.300 | 1.00 | 43.24 |
| ATOM | 7832 | NZ | LYS | B | 250 | 45.086 | 66.930 | 61.773 | 1.00 | 45.60 |
| ATOM | 7833 | N | THR | B | 251 | 46.601 | 65.700 | 56.025 | 1.00 | 28.46 |
| ATOM | 7834 | CA | THR | B | 251 | 47.616 | 66.698 | 55.877 | 1.00 | 28.16 |
| ATOM | 7835 | C | THR | B | 251 | 47.671 | 67.550 | 57.163 | 1.00 | 27.00 |
| ATOM | 7836 | O | THR | B | 251 | 46.720 | 68.218 | 57.493 | 1.00 | 27.97 |
| ATOM | 7837 | CB | THR | B | 251 | 47.323 | 67.598 | 54.675 | 1.00 | 28.23 |
| ATOM | 7838 | OG1 | THR | B | 251 | 47.486 | 66.860 | 53.467 | 1.00 | 27.70 |
| ATOM | 7839 | CG2 | THR | B | 251 | 48.390 | 68.676 | 54.558 | 1.00 | 28.77 |
| ATOM | 7840 | N | VAL | B | 252 | 48.786 | 67.493 | 57.873 | 1.00 | 25.71 |
| ATOM | 7841 | CA | VAL | B | 252 | 48.999 | 68.270 | 59.078 | 1.00 | 25.09 |
| ATOM | 7842 | C | VAL | B | 252 | 49.496 | 69.653 | 58.664 | 1.00 | 25.01 |
| ATOM | 7843 | O | VAL | B | 252 | 50.368 | 69.785 | 57.804 | 1.00 | 23.34 |
| ATOM | 7844 | CB | VAL | B | 252 | 50.022 | 67.564 | 59.975 | 1.00 | 25.60 |
| ATOM | 7845 | CG1 | VAL | B | 252 | 50.202 | 68.289 | 61.320 | 1.00 | 25.24 |
| ATOM | 7846 | CG2 | VAL | B | 252 | 49.564 | 66.168 | 60.247 | 1.00 | 26.76 |
| ATOM | 7847 | N | ARG | B | 253 | 48.889 | 70.684 | 59.224 | 1.00 | 24.91 |
| ATOM | 7848 | CA | ARG | B | 253 | 49.261 | 72.040 | 58.915 | 1.00 | 26.21 |
| ATOM | 7849 | C | ARG | B | 253 | 49.566 | 72.741 | 60.236 | 1.00 | 25.85 |
| ATOM | 7850 | O | ARG | B | 253 | 48.699 | 72.826 | 61.087 | 1.00 | 26.18 |
| ATOM | 7851 | CB | ARG | B | 253 | 48.141 | 72.761 | 58.151 | 1.00 | 26.65 |
| ATOM | 7852 | CG | ARG | B | 253 | 47.931 | 72.258 | 56.759 | 1.00 | 31.04 |
| ATOM | 7853 | CD | ARG | B | 253 | 46.673 | 72.756 | 56.041 | 1.00 | 36.68 |
| ATOM | 7854 | NE | ARG | B | 253 | 45.671 | 71.694 | 56.094 | 1.00 | 45.06 |
| ATOM | 7855 | CZ | ARG | B | 253 | 45.255 | 70.954 | 55.056 | 1.00 | 48.15 |
| ATOM | 7856 | NH1 | ARG | B | 253 | 45.699 | 71.157 | 53.804 | 1.00 | 47.19 |
| ATOM | 7857 | NH2 | ARG | B | 253 | 44.366 | 70.001 | 55.287 | 1.00 | 48.13 |
| ATOM | 7858 | N | VAL | B | 254 | 50.808 | 73.190 | 60.400 | 1.00 | 25.30 |
| ATOM | 7859 | CA | VAL | B | 254 | 51.248 | 73.868 | 61.599 | 1.00 | 25.01 |
| ATOM | 7860 | C | VAL | B | 254 | 51.866 | 75.230 | 61.307 | 1.00 | 25.27 |
| ATOM | 7861 | O | VAL | B | 254 | 52.764 | 75.357 | 60.500 | 1.00 | 24.30 |
| ATOM | 7862 | CB | VAL | B | 254 | 52.329 | 73.064 | 62.258 | 1.00 | 24.74 |
| ATOM | 7863 | CG1 | VAL | B | 254 | 52.731 | 73.675 | 63.653 | 1.00 | 26.20 |
| ATOM | 7864 | CG2 | VAL | B | 254 | 51.902 | 71.651 | 62.386 | 1.00 | 24.36 |
| ATOM | 7865 | N | PRO | B | 255 | 51.413 | 76.257 | 61.990 | 1.00 | 25.74 |
| ATOM | 7866 | CA | PRO | B | 255 | 52.028 | 77.582 | 61.825 | 1.00 | 26.05 |
| ATOM | 7867 | C | PRO | B | 255 | 53.450 | 77.486 | 62.374 | 1.00 | 26.44 |
| ATOM | 7868 | O | PRO | B | 255 | 53.583 | 77.224 | 63.562 | 1.00 | 25.74 |
| ATOM | 7869 | CB | PRO | B | 255 | 51.156 | 78.486 | 62.661 | 1.00 | 26.77 |
| ATOM | 7870 | CG | PRO | B | 255 | 49.849 | 77.683 | 62.869 | 1.00 | 27.04 |
| ATOM | 7871 | CD | PRO | B | 255 | 50.316 | 76.248 | 62.959 | 1.00 | 26.03 |
| ATOM | 7872 | N | TYR | B | 256 | 54.467 | 77.610 | 61.506 | 1.00 | 25.43 |
| ATOM | 7873 | CA | TYR | B | 256 | 55.881 | 77.432 | 61.864 | 1.00 | 23.71 |
| ATOM | 7874 | C | TYR | B | 256 | 56.741 | 78.390 | 61.070 | 1.00 | 23.86 |
| ATOM | 7875 | O | TYR | B | 256 | 56.866 | 78.242 | 59.847 | 1.00 | 23.06 |
| ATOM | 7876 | CB | TYR | B | 256 | 56.275 | 76.019 | 61.451 | 1.00 | 24.30 |
| ATOM | 7877 | CG | TYR | B | 256 | 57.692 | 75.536 | 61.692 | 1.00 | 23.12 |
| ATOM | 7878 | CD1 | TYR | B | 256 | 58.773 | 76.045 | 60.985 | 1.00 | 23.72 |
| ATOM | 7879 | CD2 | TYR | B | 256 | 57.929 | 74.518 | 62.579 | 1.00 | 22.68 |
| ATOM | 7880 | CE1 | TYR | B | 256 | 60.066 | 75.552 | 61.179 | 1.00 | 22.28 |
| ATOM | 7881 | CE2 | TYR | B | 256 | 59.194 | 74.015 | 62.786 | 1.00 | 24.55 |
| ATOM | 7882 | CZ | TYR | B | 256 | 60.262 | 74.531 | 62.088 | 1.00 | 25.38 |
| ATOM | 7883 | OH | TYR | B | 256 | 61.499 | 74.011 | 62.310 | 1.00 | 22.59 |
| ATOM | 7884 | N | PRO | B | 257 | 57.347 | 79.378 | 61.732 | 1.00 | 23.65 |
| ATOM | 7885 | CA | PRO | B | 257 | 58.211 | 80.350 | 61.038 | 1.00 | 23.40 |
| ATOM | 7886 | C | PRO | B | 257 | 59.554 | 79.785 | 60.746 | 1.00 | 22.75 |
| ATOM | 7887 | O | PRO | B | 257 | 60.275 | 79.547 | 61.674 | 1.00 | 24.71 |
| ATOM | 7888 | CB | PRO | B | 257 | 58.429 | 81.473 | 62.068 | 1.00 | 24.29 |
| ATOM | 7889 | CG | PRO | B | 257 | 58.042 | 80.886 | 63.474 | 1.00 | 24.80 |
| ATOM | 7890 | CD | PRO | B | 257 | 57.282 | 79.604 | 63.181 | 1.00 | 24.55 |
| ATOM | 7891 | N | LYS | B | 258 | 59.874 | 79.580 | 59.491 | 1.00 | 23.80 |
| ATOM | 7892 | CA | LYS | B | 258 | 61.198 | 79.199 | 59.030 | 1.00 | 23.19 |
| ATOM | 7893 | C | LYS | B | 258 | 62.111 | 80.462 | 59.053 | 1.00 | 24.02 |
| ATOM | 7894 | O | LYS | B | 258 | 61.674 | 81.591 | 59.259 | 1.00 | 20.36 |
| ATOM | 7895 | CB | LYS | B | 258 | 61.108 | 78.534 | 57.642 | 1.00 | 23.36 |
| ATOM | 7896 | CG | LYS | B | 258 | 60.637 | 77.040 | 57.673 | 1.00 | 22.40 |
| ATOM | 7897 | CD | LYS | B | 258 | 60.277 | 76.473 | 56.281 | 1.00 | 23.16 |
| ATOM | 7898 | CE | LYS | B | 258 | 59.820 | 74.992 | 56.340 | 1.00 | 22.40 |
| ATOM | 7899 | NZ | LYS | B | 258 | 60.874 | 74.018 | 56.757 | 1.00 | 21.29 |
| ATOM | 7900 | N | ALA | B | 259 | 63.407 | 80.229 | 58.956 | 1.00 | 26.33 |
| ATOM | 7901 | CA | ALA | B | 259 | 64.377 | 81.292 | 59.168 | 1.00 | 27.36 |
| ATOM | 7902 | C | ALA | B | 259 | 64.066 | 82.392 | 58.213 | 1.00 | 28.09 |
| ATOM | 7903 | O | ALA | B | 259 | 63.938 | 82.139 | 57.022 | 1.00 | 27.41 |
| ATOM | 7904 | CB | ALA | B | 259 | 65.812 | 80.758 | 58.966 | 1.00 | 27.89 |
| ATOM | 7905 | N | GLY | B | 260 | 63.868 | 83.602 | 58.749 | 1.00 | 28.60 |
| ATOM | 7906 | CA | GLY | B | 260 | 63.602 | 84.769 | 57.932 | 1.00 | 28.14 |
| ATOM | 7907 | C | GLY | B | 260 | 62.147 | 85.007 | 57.621 | 1.00 | 28.41 |
| ATOM | 7908 | O | GLY | B | 260 | 61.799 | 86.031 | 57.026 | 1.00 | 28.16 |
| ATOM | 7909 | N | ALA | B | 261 | 61.280 | 84.100 | 58.039 | 1.00 | 27.69 |
| ATOM | 7910 | CA | ALA | B | 261 | 59.862 | 84.234 | 57.713 | 1.00 | 28.41 |
| ATOM | 7911 | C | ALA | B | 261 | 59.130 | 85.087 | 58.742 | 1.00 | 27.69 |
| ATOM | 7912 | O | ALA | B | 261 | 59.684 | 85.417 | 59.797 | 1.00 | 26.36 |
| ATOM | 7913 | CB | ALA | B | 261 | 59.209 | 82.853 | 57.611 | 1.00 | 29.03 |
| ATOM | 7914 | N | VAL | B | 262 | 57.892 | 85.463 | 58.424 | 1.00 | 26.40 |
| ATOM | 7915 | CA | VAL | B | 262 | 57.100 | 86.220 | 59.364 | 1.00 | 26.14 |
| ATOM | 7916 | C | VAL | B | 262 | 56.942 | 85.380 | 60.658 | 1.00 | 26.40 |
| ATOM | 7917 | O | VAL | B | 262 | 56.500 | 84.251 | 60.592 | 1.00 | 25.67 |
| ATOM | 7918 | CB | VAL | B | 262 | 55.689 | 86.556 | 58.816 | 1.00 | 26.61 |
| ATOM | 7919 | CG1 | VAL | B | 262 | 54.787 | 87.059 | 59.912 | 1.00 | 26.64 |
| ATOM | 7920 | CG2 | VAL | B | 262 | 55.718 | 87.604 | 57.635 | 1.00 | 27.06 |
| ATOM | 7921 | N | ASN | B | 263 | 57.300 | 85.950 | 61.815 | 1.00 | 25.81 |
| ATOM | 7922 | CA | ASN | B | 263 | 57.142 | 85.313 | 63.115 | 1.00 | 26.49 |
| ATOM | 7923 | C | ASN | B | 263 | 55.807 | 85.708 | 63.721 | 1.00 | 25.87 |
| ATOM | 7924 | O | ASN | B | 263 | 55.209 | 86.670 | 63.280 | 1.00 | 26.72 |
| ATOM | 7925 | CB | ASN | B | 263 | 58.196 | 85.859 | 64.083 | 1.00 | 26.60 |
| ATOM | 7926 | CG | ASN | B | 263 | 59.444 | 85.013 | 64.177 | 1.00 | 27.28 |
| ATOM | 7927 | OD1 | ASN | B | 263 | 60.455 | 85.469 | 64.758 | 1.00 | 33.18 |
| ATOM | 7928 | ND2 | ASN | B | 263 | 59.415 | 83.804 | 63.640 | 1.00 | 18.52 |
| ATOM | 7929 | N | PRO | B | 264 | 55.324 | 84.997 | 64.732 | 1.00 | 25.50 |
| ATOM | 7930 | CA | PRO | B | 264 | 54.143 | 85.442 | 65.463 | 1.00 | 25.63 |
| ATOM | 7931 | C | PRO | B | 264 | 54.432 | 86.709 | 66.282 | 1.00 | 26.29 |
| ATOM | 7932 | O | PRO | B | 264 | 55.572 | 86.954 | 66.662 | 1.00 | 25.52 |
| ATOM | 7933 | CB | PRO | B | 264 | 53.940 | 84.314 | 66.460 | 1.00 | 26.54 |
| ATOM | 7934 | CG | PRO | B | 264 | 55.338 | 83.804 | 66.699 | 1.00 | 24.95 |
| ATOM | 7935 | CD | PRO | B | 264 | 55.846 | 83.738 | 65.286 | 1.00 | 25.96 |
| ATOM | 7936 | N | THR | B | 265 | 53.424 | 87.516 | 66.550 | 1.00 | 26.67 |
| ATOM | 7937 | CA | THR | B | 265 | 53.621 | 88.650 | 67.431 | 1.00 | 27.04 |
| ATOM | 7938 | C | THR | B | 265 | 53.054 | 88.264 | 68.773 | 1.00 | 26.93 |
| ATOM | 7939 | O | THR | B | 265 | 52.300 | 87.304 | 68.888 | 1.00 | 24.94 |
| ATOM | 7940 | CB | THR | B | 265 | 52.860 | 89.840 | 66.942 | 1.00 | 27.26 |
| ATOM | 7941 | OG1 | THR | B | 265 | 51.525 | 89.412 | 66.683 | 1.00 | 25.84 |
| ATOM | 7942 | CG2 | THR | B | 265 | 53.422 | 90.368 | 65.611 | 1.00 | 28.09 |
| ATOM | 7943 | N | VAL | B | 266 | 53.357 | 89.073 | 69.779 | 1.00 | 27.81 |
| ATOM | 7944 | CA | VAL | B | 266 | 52.907 | 88.766 | 71.137 | 1.00 | 28.12 |
| ATOM | 7945 | C | VAL | B | 266 | 52.476 | 89.967 | 71.903 | 1.00 | 27.91 |
| ATOM | 7946 | O | VAL | B | 266 | 52.986 | 91.042 | 71.695 | 1.00 | 28.76 |
| ATOM | 7947 | CB | VAL | B | 266 | 54.032 | 88.068 | 71.923 | 1.00 | 28.38 |
| ATOM | 7948 | CG1 | VAL | B | 266 | 55.318 | 88.852 | 71.856 | 1.00 | 30.25 |
| ATOM | 7949 | CG2 | VAL | B | 266 | 53.630 | 87.871 | 73.366 | 1.00 | 29.69 |
| ATOM | 7950 | N | LYS | B | 267 | 51.524 | 89.769 | 72.808 | 1.00 | 28.94 |
| ATOM | 7951 | CA | LYS | B | 267 | 50.987 | 90.823 | 73.663 | 1.00 | 29.03 |
| ATOM | 7952 | C | LYS | B | 267 | 50.978 | 90.238 | 75.054 | 1.00 | 28.26 |
| ATOM | 7953 | O | LYS | B | 267 | 50.955 | 89.011 | 75.206 | 1.00 | 26.79 |
| ATOM | 7954 | CB | LYS | B | 267 | 49.556 | 91.216 | 73.258 | 1.00 | 29.74 |
| ATOM | 7955 | CG | LYS | B | 267 | 49.404 | 92.074 | 71.966 | 1.00 | 34.60 |
| ATOM | 7956 | CD | LYS | B | 267 | 50.108 | 93.458 | 72.157 | 1.00 | 40.68 |
| ATOM | 7957 | CE | LYS | B | 267 | 49.430 | 94.624 | 71.317 | 1.00 | 44.72 |
| ATOM | 7958 | NZ | LYS | B | 267 | 49.882 | 96.041 | 71.682 | 1.00 | 42.65 |
| ATOM | 7959 | N | PHE | B | 268 | 51.020 | 91.112 | 76.060 | 1.00 | 28.28 |
| ATOM | 7960 | CA | PHE | B | 268 | 51.060 | 90.697 | 77.453 | 1.00 | 28.24 |
| ATOM | 7961 | C | PHE | B | 268 | 50.017 | 91.429 | 78.247 | 1.00 | 28.18 |
| ATOM | 7962 | O | PHE | B | 268 | 49.842 | 92.624 | 78.075 | 1.00 | 28.10 |
| ATOM | 7963 | CB | PHE | B | 268 | 52.466 | 90.919 | 78.041 | 1.00 | 28.72 |
| ATOM | 7964 | CG | PHE | B | 268 | 52.652 | 90.334 | 79.425 | 1.00 | 27.25 |
| ATOM | 7965 | CD1 | PHE | B | 268 | 53.019 | 89.030 | 79.583 | 1.00 | 26.79 |
| ATOM | 7966 | CD2 | PHE | B | 268 | 52.450 | 91.100 | 80.539 | 1.00 | 28.85 |
| ATOM | 7967 | CE1 | PHE | B | 268 | 53.180 | 88.488 | 80.803 | 1.00 | 26.99 |
| ATOM | 7968 | CE2 | PHE | B | 268 | 52.635 | 90.579 | 81.789 | 1.00 | 28.72 |
| ATOM | 7969 | CZ | PHE | B | 268 | 52.991 | 89.250 | 81.922 | 1.00 | 29.46 |
| ATOM | 7970 | N | PHE | B | 269 | 49.342 | 90.707 | 79.138 | 1.00 | 28.38 |
| ATOM | 7971 | CA | PHE | B | 269 | 48.229 | 91.238 | 79.890 | 1.00 | 29.07 |
| ATOM | 7972 | C | PHE | B | 269 | 48.189 | 90.753 | 81.329 | 1.00 | 30.14 |
| ATOM | 7973 | O | PHE | B | 269 | 48.738 | 89.698 | 81.678 | 1.00 | 29.39 |
| ATOM | 7974 | CB | PHE | B | 269 | 46.912 | 90.772 | 79.266 | 1.00 | 29.33 |
| ATOM | 7975 | CG | PHE | B | 269 | 46.636 | 91.318 | 77.884 | 1.00 | 29.82 |
| ATOM | 7976 | CD1 | PHE | B | 269 | 46.060 | 92.573 | 77.703 | 1.00 | 31.70 |
| ATOM | 7977 | CD2 | PHE | B | 269 | 46.901 | 90.555 | 76.770 | 1.00 | 27.28 |
| ATOM | 7978 | CE1 | PHE | B | 269 | 45.751 | 93.025 | 76.404 | 1.00 | 31.90 |
| ATOM | 7979 | CE2 | PHE | B | 269 | 46.617 | 91.011 | 75.522 | 1.00 | 27.59 |
| ATOM | 7980 | CZ | PHE | B | 269 | 46.041 | 92.249 | 75.338 | 1.00 | 29.73 |
| ATOM | 7981 | N | VAL | B | 270 | 47.467 | 91.491 | 82.166 | 1.00 | 31.12 |
| ATOM | 7982 | CA | VAL | B | 270 | 47.295 | 91.070 | 83.544 | 1.00 | 32.19 |
| ATOM | 7983 | C | VAL | B | 270 | 45.900 | 91.401 | 83.950 | 1.00 | 32.90 |
| ATOM | 7984 | O | VAL | B | 270 | 45.427 | 92.487 | 83.686 | 1.00 | 32.40 |
| ATOM | 7985 | CB | VAL | B | 270 | 48.222 | 91.816 | 84.463 | 1.00 | 32.52 |
| ATOM | 7986 | CG1 | VAL | B | 270 | 48.212 | 91.172 | 85.864 | 1.00 | 32.49 |
| ATOM | 7987 | CG2 | VAL | B | 270 | 49.631 | 91.835 | 83.863 | 1.00 | 32.85 |
| ATOM | 7988 | N | VAL | B | 271 | 45.259 | 90.453 | 84.604 | 1.00 | 33.75 |
| ATOM | 7989 | CA | VAL | B | 271 | 43.900 | 90.604 | 85.044 | 1.00 | 35.00 |
| ATOM | 7990 | C | VAL | B | 271 | 43.826 | 90.377 | 86.539 | 1.00 | 35.04 |
| ATOM | 7991 | O | VAL | B | 271 | 44.457 | 89.457 | 87.045 | 1.00 | 33.68 |
| ATOM | 7992 | CB | VAL | B | 271 | 43.025 | 89.491 | 84.457 | 1.00 | 35.21 |
| ATOM | 7993 | CG1 | VAL | B | 271 | 41.595 | 89.679 | 84.869 | 1.00 | 35.84 |
| ATOM | 7994 | CG2 | VAL | B | 271 | 43.153 | 89.438 | 82.966 | 1.00 | 37.46 |
| ATOM | 7995 | N | ASN | B | 272 | 43.011 | 91.177 | 87.217 | 1.00 | 36.31 |
| ATOM | 7996 | CA | ASN | B | 272 | 42.713 | 90.975 | 88.646 | 1.00 | 37.31 |
| ATOM | 7997 | C | ASN | B | 272 | 41.664 | 89.919 | 88.809 | 1.00 | 37.75 |
| ATOM | 7998 | O | ASN | B | 272 | 40.532 | 90.091 | 88.427 | 1.00 | 37.06 |
| ATOM | 7999 | CB | ASN | B | 272 | 42.178 | 92.246 | 89.300 | 1.00 | 38.06 |
| ATOM | 8000 | CG | ASN | B | 272 | 42.238 | 92.185 | 90.817 | 1.00 | 37.62 |
| ATOM | 8001 | OD1 | ASN | B | 272 | 41.886 | 91.172 | 91.434 | 1.00 | 37.86 |
| ATOM | 8002 | ND2 | ASN | B | 272 | 42.694 | 93.254 | 91.417 | 1.00 | 35.80 |
| ATOM | 8003 | N | THR | B | 273 | 42.056 | 88.842 | 89.452 | 1.00 | 39.53 |
| ATOM | 8004 | CA | THR | B | 273 | 41.240 | 87.665 | 89.632 | 1.00 | 40.60 |
| ATOM | 8005 | C | THR | B | 273 | 40.280 | 87.769 | 90.834 | 1.00 | 42.89 |
| ATOM | 8006 | O | THR | B | 273 | 39.364 | 86.948 | 90.999 | 1.00 | 41.51 |
| ATOM | 8007 | CB | THR | B | 273 | 42.238 | 86.518 | 89.758 | 1.00 | 40.45 |
| ATOM | 8008 | OG1 | THR | B | 273 | 42.157 | 85.649 | 88.612 | 1.00 | 43.84 |
| ATOM | 8009 | CG2 | THR | B | 273 | 42.034 | 85.673 | 90.930 | 1.00 | 38.99 |
| ATOM | 8010 | N | ASP | B | 274 | 40.479 | 88.789 | 91.661 | 1.00 | 45.49 |
| ATOM | 8011 | CA | ASP | B | 274 | 39.691 | 88.935 | 92.889 | 1.00 | 48.12 |
| ATOM | 8012 | C | ASP | B | 274 | 38.436 | 89.759 | 92.620 | 1.00 | 49.85 |
| ATOM | 8013 | O | ASP | B | 274 | 37.487 | 89.750 | 93.406 | 1.00 | 49.50 |
| ATOM | 8014 | CB | ASP | B | 274 | 40.533 | 89.570 | 94.003 | 1.00 | 48.46 |
| ATOM | 8015 | CG | ASP | B | 274 | 41.502 | 88.578 | 94.648 | 1.00 | 49.63 |
| ATOM | 8016 | OD1 | ASP | B | 274 | 41.248 | 87.349 | 94.604 | 1.00 | 47.99 |
| ATOM | 8017 | OD2 | ASP | B | 274 | 42.543 | 88.951 | 95.240 | 1.00 | 52.84 |
| ATOM | 8018 | N | SER | B | 275 | 38.425 | 90.433 | 91.472 | 1.00 | 51.84 |
| ATOM | 8019 | CA | SER | B | 275 | 37.279 | 91.222 | 91.063 | 1.00 | 52.96 |
| ATOM | 8020 | C | SER | B | 275 | 36.699 | 90.683 | 89.775 | 1.00 | 54.06 |
| ATOM | 8021 | O | SER | B | 275 | 36.981 | 91.222 | 88.705 | 1.00 | 55.29 |
| ATOM | 8022 | CB | SER | B | 275 | 37.721 | 92.661 | 90.817 | 1.00 | 53.02 |
| ATOM | 8023 | OG | SER | B | 275 | 38.518 | 92.734 | 89.656 | 1.00 | 52.54 |
| ATOM | 8024 | N | LEU | B | 276 | 35.908 | 89.623 | 89.852 | 1.00 | 54.49 |
| ATOM | 8025 | CA | LEU | B | 276 | 35.311 | 89.070 | 88.649 | 1.00 | 54.65 |
| ATOM | 8026 | C | LEU | B | 276 | 33.824 | 88.957 | 88.839 | 1.00 | 54.97 |
| ATOM | 8027 | O | LEU | B | 276 | 33.358 | 88.767 | 89.945 | 1.00 | 54.57 |
| ATOM | 8028 | CB | LEU | B | 276 | 35.909 | 87.707 | 88.312 | 1.00 | 54.66 |
| ATOM | 8029 | CG | LEU | B | 276 | 37.364 | 87.736 | 87.872 | 1.00 | 53.89 |
| ATOM | 8030 | CD1 | LEU | B | 276 | 37.878 | 86.324 | 87.751 | 1.00 | 54.37 |
| ATOM | 8031 | CD2 | LEU | B | 276 | 37.527 | 88.496 | 86.575 | 1.00 | 51.77 |
| ATOM | 8032 | N | SER | B | 277 | 33.087 | 89.068 | 87.741 | 1.00 | 55.99 |
| ATOM | 8033 | CA | SER | B | 277 | 31.628 | 89.074 | 87.782 | 1.00 | 56.29 |
| ATOM | 8034 | C | SER | B | 277 | 30.999 | 87.959 | 86.971 | 1.00 | 56.82 |
| ATOM | 8035 | O | SER | B | 277 | 31.382 | 87.717 | 85.826 | 1.00 | 56.20 |
| ATOM | 8036 | CB | SER | B | 277 | 31.114 | 90.397 | 87.257 | 1.00 | 56.23 |
| ATOM | 8037 | OG | SER | B | 277 | 29.747 | 90.266 | 86.932 | 1.00 | 56.57 |
| ATOM | 8038 | N | SER | B | 278 | 29.994 | 87.327 | 87.569 | 1.00 | 57.97 |
| ATOM | 8039 | CA | SER | B | 278 | 29.334 | 86.171 | 86.985 | 1.00 | 58.95 |
| ATOM | 8040 | C | SER | B | 278 | 28.986 | 86.391 | 85.543 | 1.00 | 59.89 |
| ATOM | 8041 | O | SER | B | 278 | 29.183 | 85.492 | 84.726 | 1.00 | 60.37 |
| ATOM | 8042 | CB | SER | B | 278 | 28.078 | 85.777 | 87.775 | 1.00 | 59.11 |
| ATOM | 8043 | OG | SER | B | 278 | 28.378 | 84.786 | 88.768 | 1.00 | 59.81 |
| ATOM | 8044 | N | VAL | B | 279 | 28.496 | 87.583 | 85.210 | 1.00 | 60.61 |
| ATOM | 8045 | CA | VAL | B | 279 | 28.110 | 87.840 | 83.829 | 1.00 | 61.05 |
| ATOM | 8046 | C | VAL | B | 279 | 28.938 | 88.901 | 83.086 | 1.00 | 60.36 |
| ATOM | 8047 | O | VAL | B | 279 | 29.131 | 88.783 | 81.869 | 1.00 | 61.06 |
| ATOM | 8048 | CB | VAL | B | 279 | 26.623 | 88.223 | 83.723 | 1.00 | 61.89 |
| ATOM | 8049 | CG1 | VAL | B | 279 | 26.075 | 87.713 | 82.398 | 1.00 | 63.23 |
| ATOM | 8050 | CG2 | VAL | B | 279 | 25.816 | 87.640 | 84.889 | 1.00 | 62.56 |
| ATOM | 8051 | N | THR | B | 280 | 29.422 | 89.920 | 83.800 | 1.00 | 59.14 |
| ATOM | 8052 | CA | THR | B | 280 | 30.189 | 91.037 | 83.197 | 1.00 | 57.83 |
| ATOM | 8053 | C | THR | B | 280 | 31.635 | 90.680 | 82.805 | 1.00 | 56.12 |
| ATOM | 8054 | O | THR | B | 280 | 32.362 | 90.043 | 83.576 | 1.00 | 56.22 |
| ATOM | 8055 | CB | THR | B | 280 | 30.174 | 92.238 | 84.170 | 1.00 | 58.21 |
| ATOM | 8056 | OG1 | THR | B | 280 | 28.829 | 92.709 | 84.301 | 1.00 | 57.77 |
| ATOM | 8057 | CG2 | THR | B | 280 | 30.957 | 93.466 | 83.618 | 1.00 | 58.84 |
| ATOM | 8058 | N | ASN | B | 281 | 32.039 | 91.121 | 81.613 | 1.00 | 54.01 |
| ATOM | 8059 | CA | ASN | B | 281 | 33.341 | 90.780 | 81.024 | 1.00 | 52.59 |
| ATOM | 8060 | C | ASN | B | 281 | 34.552 | 91.311 | 81.799 | 1.00 | 51.17 |
| ATOM | 8061 | O | ASN | B | 281 | 34.623 | 92.490 | 82.136 | 1.00 | 51.36 |
| ATOM | 8062 | CB | ASN | B | 281 | 33.381 | 91.205 | 79.543 | 1.00 | 52.22 |
| ATOM | 8063 | CG | ASN | B | 281 | 32.565 | 90.259 | 78.649 | 1.00 | 53.14 |
| ATOM | 8064 | OD1 | ASN | B | 281 | 32.162 | 89.177 | 79.103 | 1.00 | 51.75 |
| ATOM | 8065 | ND2 | ASN | B | 281 | 32.329 | 90.646 | 77.377 | 1.00 | 50.81 |
| ATOM | 8066 | N | ALA | B | 282 | 35.495 | 90.416 | 82.083 | 1.00 | 49.39 |
| ATOM | 8067 | CA | ALA | B | 282 | 36.697 | 90.743 | 82.859 | 1.00 | 48.04 |
| ATOM | 8068 | C | ALA | B | 282 | 37.609 | 91.717 | 82.128 | 1.00 | 46.65 |
| ATOM | 8069 | O | ALA | B | 282 | 37.724 | 91.654 | 80.906 | 1.00 | 45.49 |
| ATOM | 8070 | CB | ALA | B | 282 | 37.459 | 89.473 | 83.164 | 1.00 | 48.05 |
| ATOM | 8071 | N | THR | B | 283 | 38.272 | 92.609 | 82.858 | 1.00 | 45.32 |
| ATOM | 8072 | CA | THR | B | 283 | 39.188 | 93.534 | 82.193 | 1.00 | 45.17 |
| ATOM | 8073 | C | THR | B | 283 | 40.644 | 93.048 | 82.252 | 1.00 | 43.92 |
| ATOM | 8074 | O | THR | B | 283 | 41.140 | 92.684 | 83.304 | 1.00 | 45.06 |
| ATOM | 8075 | CB | THR | B | 283 | 39.093 | 94.945 | 82.806 | 1.00 | 45.56 |
| ATOM | 8076 | OG1 | THR | B | 283 | 37.791 | 95.509 | 82.557 | 1.00 | 47.54 |
| ATOM | 8077 | CG2 | THR | B | 283 | 40.033 | 95.895 | 82.089 | 1.00 | 45.76 |
| ATOM | 8078 | N | SER | B | 284 | 41.311 | 93.030 | 81.117 | 1.00 | 41.77 |
| ATOM | 8079 | CA | SER | B | 284 | 42.708 | 92.665 | 81.049 | 1.00 | 40.86 |
| ATOM | 8080 | C | SER | B | 284 | 43.529 | 93.929 | 80.874 | 1.00 | 39.75 |
| ATOM | 8081 | O | SER | B | 284 | 43.259 | 94.700 | 79.963 | 1.00 | 39.36 |
| ATOM | 8082 | CB | SER | B | 284 | 42.952 | 91.755 | 79.838 | 1.00 | 40.93 |
| ATOM | 8083 | OG | SER | B | 284 | 42.604 | 90.405 | 80.107 | 1.00 | 40.28 |
| ATOM | 8084 | N | ILE | B | 285 | 44.508 | 94.182 | 81.734 | 1.00 | 38.45 |
| ATOM | 8085 | CA | ILE | B | 285 | 45.354 | 95.348 | 81.495 | 1.00 | 37.90 |
| ATOM | 8086 | C | ILE | B | 285 | 46.556 | 94.957 | 80.677 | 1.00 | 36.75 |
| ATOM | 8087 | O | ILE | B | 285 | 47.196 | 93.952 | 80.958 | 1.00 | 36.40 |
| ATOM | 8088 | CB | ILE | B | 285 | 45.752 | 96.057 | 82.781 | 1.00 | 37.70 |
| ATOM | 8089 | CG1 | ILE | B | 285 | 44.512 | 96.661 | 83.435 | 1.00 | 39.24 |
| ATOM | 8090 | CG2 | ILE | B | 285 | 46.701 | 97.209 | 82.477 | 1.00 | 37.80 |
| ATOM | 8091 | CD1 | ILE | B | 285 | 44.009 | 95.843 | 84.572 | 1.00 | 40.83 |
| ATOM | 8092 | N | GLN | B | 286 | 46.848 | 95.739 | 79.639 | 1.00 | 36.28 |
| ATOM | 8093 | CA | GLN | B | 286 | 47.933 | 95.408 | 78.741 | 1.00 | 36.02 |
| ATOM | 8094 | C | GLN | B | 286 | 49.270 | 96.017 | 79.167 | 1.00 | 36.44 |
| ATOM | 8095 | O | GLN | B | 286 | 49.335 | 97.183 | 79.507 | 1.00 | 35.96 |
| ATOM | 8096 | CB | GLN | B | 286 | 47.611 | 95.830 | 77.294 | 1.00 | 35.59 |
| ATOM | 8097 | CG | GLN | B | 286 | 48.760 | 95.542 | 76.284 | 1.00 | 34.01 |
| ATOM | 8098 | CD | GLN | B | 286 | 48.368 | 95.650 | 74.794 | 1.00 | 30.38 |
| ATOM | 8099 | OE1 | GLN | B | 286 | 47.325 | 96.170 | 74.437 | 1.00 | 32.82 |
| ATOM | 8100 | NE2 | GLN | B | 286 | 49.197 | 95.133 | 73.951 | 1.00 | 27.98 |
| ATOM | 8101 | N | ILE | B | 287 | 50.341 | 95.226 | 79.162 | 1.00 | 36.19 |
| ATOM | 8102 | CA | ILE | B | 287 | 51.628 | 95.820 | 79.365 | 1.00 | 36.28 |
| ATOM | 8103 | C | ILE | B | 287 | 52.345 | 95.765 | 78.029 | 1.00 | 36.85 |
| ATOM | 8104 | O | ILE | B | 287 | 52.539 | 94.720 | 77.428 | 1.00 | 36.85 |
| ATOM | 8105 | CB | ILE | B | 287 | 52.455 | 95.182 | 80.477 | 1.00 | 36.53 |
| ATOM | 8106 | CG1 | ILE | B | 287 | 51.718 | 95.200 | 81.808 | 1.00 | 36.38 |
| ATOM | 8107 | CG2 | ILE | B | 287 | 53.784 | 95.966 | 80.636 | 1.00 | 36.09 |
| ATOM | 8108 | CD1 | ILE | B | 287 | 52.495 | 94.510 | 82.943 | 1.00 | 37.45 |
| ATOM | 8109 | N | THR | B | 288 | 52.697 | 96.936 | 77.551 | 1.00 | 37.71 |
| ATOM | 8110 | CA | THR | B | 288 | 53.355 | 97.092 | 76.274 | 1.00 | 38.55 |
| ATOM | 8111 | C | THR | B | 288 | 54.865 | 96.886 | 76.355 | 1.00 | 37.97 |
| ATOM | 8112 | O | THR | B | 288 | 55.512 | 97.302 | 77.281 | 1.00 | 37.22 |
| ATOM | 8113 | CB | THR | B | 288 | 53.019 | 98.516 | 75.753 | 1.00 | 39.37 |
| ATOM | 8114 | OG1 | THR | B | 288 | 51.614 | 98.585 | 75.420 | 1.00 | 40.35 |
| ATOM | 8115 | CG2 | THR | B | 288 | 53.681 | 98.761 | 74.440 | 1.00 | 40.05 |
| ATOM | 8116 | N | ALA | B | 289 | 55.416 | 96.198 | 75.379 | 1.00 | 38.81 |
| ATOM | 8117 | CA | ALA | B | 289 | 56.850 | 96.042 | 75.308 | 1.00 | 39.29 |
| ATOM | 8118 | C | ALA | B | 289 | 57.457 | 97.433 | 75.256 | 1.00 | 39.54 |
| ATOM | 8119 | O | ALA | B | 289 | 56.809 | 98.366 | 74.835 | 1.00 | 39.93 |
| ATOM | 8120 | CB | ALA | B | 289 | 57.211 | 95.266 | 74.067 | 1.00 | 39.55 |
| ATOM | 8121 | N | PRO | B | 290 | 58.683 | 97.593 | 75.722 | 1.00 | 39.99 |
| ATOM | 8122 | CA | PRO | B | 290 | 59.360 | 98.887 | 75.635 | 1.00 | 40.02 |
| ATOM | 8123 | C | PRO | B | 290 | 59.585 | 99.299 | 74.214 | 1.00 | 39.92 |
| ATOM | 8124 | O | PRO | B | 290 | 59.813 | 98.446 | 73.349 | 1.00 | 39.32 |
| ATOM | 8125 | CB | PRO | B | 290 | 60.715 | 98.623 | 76.252 | 1.00 | 39.94 |
| ATOM | 8126 | CG | PRO | B | 290 | 60.505 | 97.448 | 77.064 | 1.00 | 41.06 |
| ATOM | 8127 | CD | PRO | B | 290 | 59.495 | 96.588 | 76.401 | 1.00 | 39.84 |
| ATOM | 8128 | N | ALA | B | 291 | 59.581 | 100.608 | 74.000 | 1.00 | 40.03 |
| ATOM | 8129 | CA | ALA | B | 291 | 59.780 | 101.186 | 72.681 | 1.00 | 39.44 |
| ATOM | 8130 | C | ALA | B | 291 | 61.023 | 100.669 | 72.001 | 1.00 | 38.45 |
| ATOM | 8131 | O | ALA | B | 291 | 61.044 | 100.531 | 70.790 | 1.00 | 38.60 |
| ATOM | 8132 | CB | ALA | B | 291 | 59.821 | 102.699 | 72.782 | 1.00 | 40.01 |
| ATOM | 8133 | N | SER | B | 292 | 62.071 | 100.368 | 72.744 | 1.00 | 37.54 |
| ATOM | 8134 | CA | SER | B | 292 | 63.264 | 99.846 | 72.072 | 1.00 | 37.26 |
| ATOM | 8135 | C | SER | B | 292 | 63.037 | 98.425 | 71.450 | 1.00 | 36.81 |
| ATOM | 8136 | O | SER | B | 292 | 63.881 | 97.924 | 70.690 | 1.00 | 35.22 |
| ATOM | 8137 | CB | SER | B | 292 | 64.418 | 99.814 | 73.031 | 1.00 | 37.00 |
| ATOM | 8138 | OG | SER | B | 292 | 64.198 | 98.820 | 73.985 | 1.00 | 37.49 |
| ATOM | 8139 | N | MET | B | 293 | 61.908 | 97.792 | 71.785 | 1.00 | 36.22 |
| ATOM | 8140 | CA | MET | B | 293 | 61.491 | 96.538 | 71.134 | 1.00 | 36.74 |
| ATOM | 8141 | C | MET | B | 293 | 60.449 | 96.774 | 70.041 | 1.00 | 37.20 |
| ATOM | 8142 | O | MET | B | 293 | 60.458 | 96.103 | 69.015 | 1.00 | 37.42 |
| ATOM | 8143 | CB | MET | B | 293 | 60.894 | 95.562 | 72.165 | 1.00 | 35.93 |
| ATOM | 8144 | CG | MET | B | 293 | 61.900 | 95.069 | 73.167 | 1.00 | 35.10 |
| ATOM | 8145 | SD | MET | B | 293 | 63.060 | 93.932 | 72.473 | 1.00 | 33.78 |
| ATOM | 8146 | CE | MET | B | 293 | 64.400 | 94.091 | 73.586 | 1.00 | 36.67 |
| ATOM | 8147 | N | LEU | B | 294 | 59.524 | 97.705 | 70.268 | 1.00 | 38.14 |
| ATOM | 8148 | CA | LEU | B | 294 | 58.458 | 97.958 | 69.305 | 1.00 | 38.99 |
| ATOM | 8149 | C | LEU | B | 294 | 59.023 | 98.512 | 67.991 | 1.00 | 39.37 |
| ATOM | 8150 | O | LEU | B | 294 | 58.341 | 98.590 | 66.995 | 1.00 | 39.80 |
| ATOM | 8151 | CB | LEU | B | 294 | 57.433 | 98.903 | 69.887 | 1.00 | 39.18 |
| ATOM | 8152 | CG | LEU | B | 294 | 56.705 | 98.329 | 71.123 | 1.00 | 41.58 |
| ATOM | 8153 | CD1 | LEU | B | 294 | 55.889 | 99.401 | 71.811 | 1.00 | 40.69 |
| ATOM | 8154 | CD2 | LEU | B | 294 | 55.799 | 97.135 | 70.762 | 1.00 | 41.80 |
| ATOM | 8155 | N | ILE | B | 295 | 60.271 | 98.926 | 68.034 | 1.00 | 39.37 |
| ATOM | 8156 | CA | ILE | B | 295 | 61.001 | 99.382 | 66.872 | 1.00 | 40.16 |
| ATOM | 8157 | C | ILE | B | 295 | 60.918 | 98.331 | 65.744 | 1.00 | 39.08 |
| ATOM | 8158 | O | ILE | B | 295 | 60.846 | 98.695 | 64.579 | 1.00 | 38.34 |
| ATOM | 8159 | CB | ILE | B | 295 | 62.441 | 99.617 | 67.359 | 1.00 | 40.97 |
| ATOM | 8160 | CG1 | ILE | B | 295 | 63.505 | 99.653 | 66.286 | 1.00 | 43.52 |
| ATOM | 8161 | CG2 | ILE | B | 295 | 62.868 | 98.483 | 68.240 | 1.00 | 41.91 |
| ATOM | 8162 | CD1 | ILE | B | 295 | 64.938 | 99.846 | 66.952 | 1.00 | 45.53 |
| ATOM | 8163 | N | GLY | B | 296 | 60.916 | 97.040 | 66.097 | 1.00 | 37.48 |
| ATOM | 8164 | CA | GLY | B | 296 | 60.843 | 95.967 | 65.105 | 1.00 | 37.13 |
| ATOM | 8165 | C | GLY | B | 296 | 60.356 | 94.616 | 65.635 | 1.00 | 35.92 |
| ATOM | 8166 | O | GLY | B | 296 | 59.589 | 94.573 | 66.582 | 1.00 | 35.78 |
| ATOM | 8167 | N | ASP | B | 297 | 60.779 | 93.521 | 65.013 | 1.00 | 34.38 |
| ATOM | 8168 | CA | ASP | B | 297 | 60.367 | 92.200 | 65.458 | 1.00 | 33.95 |
| ATOM | 8169 | C | ASP | B | 297 | 61.055 | 91.884 | 66.815 | 1.00 | 32.55 |
| ATOM | 8170 | O | ASP | B | 297 | 62.251 | 92.111 | 66.986 | 1.00 | 32.71 |
| ATOM | 8171 | CB | ASP | B | 297 | 60.769 | 91.133 | 64.431 | 1.00 | 34.16 |
| ATOM | 8172 | CG | ASP | B | 297 | 59.768 | 90.974 | 63.284 | 1.00 | 35.85 |
| ATOM | 8173 | OD1 | ASP | B | 297 | 58.741 | 91.689 | 63.212 | 1.00 | 38.80 |
| ATOM | 8174 | OD2 | ASP | B | 297 | 59.939 | 90.109 | 62.401 | 1.00 | 38.68 |
| ATOM | 8175 | N | HIS | B | 298 | 60.312 | 91.350 | 67.759 | 1.00 | 30.55 |
| ATOM | 8176 | CA | HIS | B | 298 | 60.874 | 91.004 | 69.054 | 1.00 | 30.71 |
| ATOM | 8177 | C | HIS | B | 298 | 60.130 | 89.772 | 69.621 | 1.00 | 29.76 |
| ATOM | 8178 | O | HIS | B | 298 | 59.090 | 89.365 | 69.063 | 1.00 | 27.72 |
| ATOM | 8179 | CB | HIS | B | 298 | 60.714 | 92.206 | 70.014 | 1.00 | 30.05 |
| ATOM | 8180 | CG | HIS | B | 298 | 59.328 | 92.743 | 70.019 | 1.00 | 31.67 |
| ATOM | 8181 | ND1 | HIS | B | 298 | 58.289 | 92.093 | 70.651 | 1.00 | 32.69 |
| ATOM | 8182 | CD2 | HIS | B | 298 | 58.778 | 93.807 | 69.387 | 1.00 | 32.54 |
| ATOM | 8183 | CE1 | HIS | B | 298 | 57.165 | 92.763 | 70.445 | 1.00 | 33.57 |
| ATOM | 8184 | NE2 | HIS | B | 298 | 57.433 | 93.807 | 69.682 | 1.00 | 32.79 |
| ATOM | 8185 | N | TYR | B | 299 | 60.646 | 89.263 | 70.761 | 1.00 | 28.58 |
| ATOM | 8186 | CA | TYR | B | 299 | 60.088 | 88.128 | 71.506 | 1.00 | 27.63 |
| ATOM | 8187 | C | TYR | B | 299 | 59.928 | 88.447 | 72.996 | 1.00 | 27.71 |
| ATOM | 8188 | O | TYR | B | 299 | 60.705 | 89.211 | 73.574 | 1.00 | 26.61 |
| ATOM | 8189 | CB | TYR | B | 299 | 61.044 | 86.939 | 71.462 | 1.00 | 27.77 |
| ATOM | 8190 | CG | TYR | B | 299 | 61.450 | 86.482 | 70.086 | 1.00 | 26.03 |
| ATOM | 8191 | CD1 | TYR | B | 299 | 60.543 | 85.858 | 69.243 | 1.00 | 25.67 |
| ATOM | 8192 | CD2 | TYR | B | 299 | 62.746 | 86.682 | 69.632 | 1.00 | 24.84 |
| ATOM | 8193 | CE1 | TYR | B | 299 | 60.930 | 85.444 | 67.989 | 1.00 | 27.49 |
| ATOM | 8194 | CE2 | TYR | B | 299 | 63.137 | 86.274 | 68.382 | 1.00 | 25.15 |
| ATOM | 8195 | CZ | TYR | B | 299 | 62.236 | 85.657 | 67.561 | 1.00 | 25.06 |
| ATOM | 8196 | OH | TYR | B | 299 | 62.642 | 85.223 | 66.324 | 1.00 | 24.17 |
| ATOM | 8197 | N | LEU | B | 300 | 58.941 | 87.821 | 73.619 | 1.00 | 28.49 |
| ATOM | 8198 | CA | LEU | B | 300 | 58.779 | 87.852 | 75.055 | 1.00 | 28.84 |
| ATOM | 8199 | C | LEU | B | 300 | 59.562 | 86.601 | 75.450 | 1.00 | 29.14 |
| ATOM | 8200 | O | LEU | B | 300 | 59.231 | 85.527 | 74.992 | 1.00 | 27.77 |
| ATOM | 8201 | CB | LEU | B | 300 | 57.322 | 87.707 | 75.434 | 1.00 | 29.23 |
| ATOM | 8202 | CG | LEU | B | 300 | 56.811 | 88.266 | 76.750 | 1.00 | 32.12 |
| ATOM | 8203 | CD1 | LEU | B | 300 | 55.589 | 87.482 | 77.288 | 1.00 | 32.90 |
| ATOM | 8204 | CD2 | LEU | B | 300 | 57.835 | 88.324 | 77.774 | 1.00 | 32.13 |
| ATOM | 8205 | N | CYS | B | 301 | 60.597 | 86.728 | 76.272 | 1.00 | 29.52 |
| ATOM | 8206 | CA | CYS | B | 301 | 61.468 | 85.588 | 76.511 | 1.00 | 31.07 |
| ATOM | 8207 | C | CYS | B | 301 | 61.295 | 85.042 | 78.009 | 1.00 | 33.70 |
| ATOM | 8208 | O | CYS | B | 301 | 61.457 | 83.843 | 78.255 | 1.00 | 35.22 |
| ATOM | 8209 | CB | CYS | B | 301 | 62.955 | 85.890 | 75.934 | 1.00 | 31.26 |
| ATOM | 8210 | SG | CYS | B | 301 | 63.459 | 85.226 | 74.152 | 1.00 | 27.63 |
| ATOM | 8211 | N | ASP | B | 302 | 60.840 | 85.858 | 78.975 | 1.00 | 35.00 |
| ATOM | 8212 | CA | ASP | B | 302 | 60.731 | 85.433 | 80.390 | 1.00 | 35.86 |
| ATOM | 8213 | C | ASP | B | 302 | 59.799 | 86.343 | 81.231 | 1.00 | 34.62 |
| ATOM | 8214 | O | ASP | B | 302 | 59.860 | 87.545 | 81.093 | 1.00 | 34.73 |
| ATOM | 8215 | CB | ASP | B | 302 | 62.139 | 85.511 | 81.022 | 1.00 | 37.25 |
| ATOM | 8216 | CG | ASP | B | 302 | 62.420 | 84.378 | 81.981 | 1.00 | 41.73 |
| ATOM | 8217 | OD1 | ASP | B | 302 | 61.930 | 84.430 | 83.144 | 1.00 | 44.78 |
| ATOM | 8218 | OD2 | ASP | B | 302 | 63.155 | 83.394 | 81.660 | 1.00 | 49.19 |
| ATOM | 8219 | N | VAL | B | 303 | 58.955 | 85.778 | 82.098 | 1.00 | 33.21 |
| ATOM | 8220 | CA | VAL | B | 303 | 58.075 | 86.546 | 82.968 | 1.00 | 32.77 |
| ATOM | 8221 | C | VAL | B | 303 | 58.258 | 86.081 | 84.410 | 1.00 | 32.40 |
| ATOM | 8222 | O | VAL | B | 303 | 58.163 | 84.890 | 84.672 | 1.00 | 31.17 |
| ATOM | 8223 | CB | VAL | B | 303 | 56.578 | 86.310 | 82.680 | 1.00 | 33.24 |
| ATOM | 8224 | CG1 | VAL | B | 303 | 55.759 | 87.164 | 83.572 | 1.00 | 32.91 |
| ATOM | 8225 | CG2 | VAL | B | 303 | 56.218 | 86.597 | 81.232 | 1.00 | 33.73 |
| ATOM | 8226 | N | THR | B | 304 | 58.471 | 87.013 | 85.343 | 1.00 | 31.14 |
| ATOM | 8227 | CA | THR | B | 304 | 58.775 | 86.624 | 86.706 | 1.00 | 31.77 |
| ATOM | 8228 | C | THR | B | 304 | 58.112 | 87.603 | 87.658 | 1.00 | 31.80 |
| ATOM | 8229 | O | THR | B | 304 | 58.304 | 88.816 | 87.523 | 1.00 | 32.38 |
| ATOM | 8230 | CB | THR | B | 304 | 60.327 | 86.611 | 86.970 | 1.00 | 31.19 |
| ATOM | 8231 | OG1 | THR | B | 304 | 60.992 | 85.722 | 86.057 | 1.00 | 32.47 |
| ATOM | 8232 | CG2 | THR | B | 304 | 60.633 | 86.006 | 88.314 | 1.00 | 34.29 |
| ATOM | 8233 | N | TRP | B | 305 | 57.327 | 87.086 | 88.603 | 1.00 | 31.45 |
| ATOM | 8234 | CA | TRP | B | 305 | 56.718 | 87.935 | 89.617 | 1.00 | 31.62 |
| ATOM | 8235 | C | TRP | B | 305 | 57.811 | 88.187 | 90.637 | 1.00 | 31.10 |
| ATOM | 8236 | O | TRP | B | 305 | 58.330 | 87.248 | 91.154 | 1.00 | 31.07 |
| ATOM | 8237 | CB | TRP | B | 305 | 55.517 | 87.225 | 90.269 | 1.00 | 31.70 |
| ATOM | 8238 | CG | TRP | B | 305 | 54.311 | 87.317 | 89.468 | 1.00 | 31.84 |
| ATOM | 8239 | CD1 | TRP | B | 305 | 53.852 | 86.405 | 88.560 | 1.00 | 33.39 |
| ATOM | 8240 | CD2 | TRP | B | 305 | 53.406 | 88.424 | 89.410 | 1.00 | 31.59 |
| ATOM | 8241 | NE1 | TRP | B | 305 | 52.711 | 86.879 | 87.959 | 1.00 | 31.28 |
| ATOM | 8242 | CE2 | TRP | B | 305 | 52.419 | 88.116 | 88.470 | 1.00 | 31.64 |
| ATOM | 8243 | CE3 | TRP | B | 305 | 53.335 | 89.644 | 90.057 | 1.00 | 33.06 |
| ATOM | 8244 | CZ2 | TRP | B | 305 | 51.377 | 88.979 | 88.178 | 1.00 | 33.53 |
| ATOM | 8245 | CZ3 | TRP | B | 305 | 52.286 | 90.497 | 89.770 | 1.00 | 34.48 |
| ATOM | 8246 | CH2 | TRP | B | 305 | 51.323 | 90.160 | 88.853 | 1.00 | 32.18 |
| ATOM | 8247 | N | ALA | B | 306 | 58.217 | 89.433 | 90.864 | 1.00 | 31.93 |
| ATOM | 8248 | CA | ALA | B | 306 | 59.201 | 89.737 | 91.912 | 1.00 | 32.77 |
| ATOM | 8249 | C | ALA | B | 306 | 58.518 | 89.898 | 93.262 | 1.00 | 33.56 |
| ATOM | 8250 | O | ALA | B | 306 | 59.051 | 89.449 | 94.250 | 1.00 | 35.17 |
| ATOM | 8251 | CB | ALA | B | 306 | 59.980 | 90.949 | 91.575 | 1.00 | 32.78 |
| ATOM | 8252 | N | THR | B | 307 | 57.365 | 90.554 | 93.297 | 1.00 | 34.17 |
| ATOM | 8253 | CA | THR | B | 307 | 56.524 | 90.673 | 94.506 | 1.00 | 35.64 |
| ATOM | 8254 | C | THR | B | 307 | 55.055 | 90.588 | 94.074 | 1.00 | 36.27 |
| ATOM | 8255 | O | THR | B | 307 | 54.780 | 90.554 | 92.869 | 1.00 | 36.59 |
| ATOM | 8256 | CB | THR | B | 307 | 56.695 | 92.039 | 95.248 | 1.00 | 35.48 |
| ATOM | 8257 | OG1 | THR | B | 307 | 56.206 | 93.080 | 94.416 | 1.00 | 34.55 |
| ATOM | 8258 | CG2 | THR | B | 307 | 58.175 | 92.410 | 95.488 | 1.00 | 36.51 |
| ATOM | 8259 | N | GLN | B | 308 | 54.120 | 90.631 | 95.034 | 1.00 | 36.50 |
| ATOM | 8260 | CA | GLN | B | 308 | 52.690 | 90.608 | 94.714 | 1.00 | 36.66 |
| ATOM | 8261 | C | GLN | B | 308 | 52.351 | 91.765 | 93.762 | 1.00 | 36.45 |
| ATOM | 8262 | O | GLN | B | 308 | 51.316 | 91.743 | 93.107 | 1.00 | 36.23 |
| ATOM | 8263 | CB | GLN | B | 308 | 51.789 | 90.732 | 95.984 | 1.00 | 37.14 |
| ATOM | 8264 | CG | GLN | B | 308 | 52.147 | 89.883 | 97.215 | 1.00 | 37.08 |
| ATOM | 8265 | CD | GLN | B | 308 | 51.937 | 88.392 | 96.987 | 1.00 | 40.81 |
| ATOM | 8266 | OE1 | GLN | B | 308 | 51.351 | 87.991 | 95.972 | 1.00 | 38.86 |
| ATOM | 8267 | NE2 | GLN | B | 308 | 52.418 | 87.562 | 97.924 | 1.00 | 37.75 |
| ATOM | 8268 | N | GLU | B | 309 | 53.212 | 92.775 | 93.688 | 1.00 | 36.25 |
| ATOM | 8269 | CA | GLU | B | 309 | 52.907 | 93.966 | 92.883 | 1.00 | 36.43 |
| ATOM | 8270 | C | GLU | B | 309 | 53.954 | 94.349 | 91.879 | 1.00 | 35.70 |
| ATOM | 8271 | O | GLU | B | 309 | 53.939 | 95.485 | 91.363 | 1.00 | 35.36 |
| ATOM | 8272 | CB | GLU | B | 309 | 52.725 | 95.188 | 93.765 | 1.00 | 36.88 |
| ATOM | 8273 | CG | GLU | B | 309 | 51.746 | 94.973 | 94.891 | 1.00 | 41.29 |
| ATOM | 8274 | CD | GLU | B | 309 | 51.386 | 96.277 | 95.577 | 1.00 | 46.84 |
| ATOM | 8275 | OE1 | GLU | B | 309 | 52.313 | 97.065 | 95.905 | 1.00 | 50.25 |
| ATOM | 8276 | OE2 | GLU | B | 309 | 50.169 | 96.513 | 95.748 | 1.00 | 51.11 |
| ATOM | 8277 | N | ARG | B | 310 | 54.873 | 93.443 | 91.588 | 1.00 | 34.99 |
| ATOM | 8278 | CA | ARG | B | 310 | 55.907 | 93.777 | 90.620 | 1.00 | 34.77 |
| ATOM | 8279 | C | ARG | B | 310 | 56.220 | 92.582 | 89.759 | 1.00 | 34.15 |
| ATOM | 8280 | O | ARG | B | 310 | 56.494 | 91.501 | 90.272 | 1.00 | 34.19 |
| ATOM | 8281 | CB | ARG | B | 310 | 57.165 | 94.261 | 91.337 | 1.00 | 34.67 |
| ATOM | 8282 | CG | ARG | B | 310 | 58.381 | 94.291 | 90.481 | 1.00 | 34.36 |
| ATOM | 8283 | CD | ARG | B | 310 | 59.598 | 94.806 | 91.231 | 1.00 | 37.03 |
| ATOM | 8284 | NE | ARG | B | 310 | 59.478 | 96.236 | 91.473 | 1.00 | 38.15 |
| ATOM | 8285 | CZ | ARG | B | 310 | 60.277 | 96.964 | 92.226 | 1.00 | 40.61 |
| ATOM | 8286 | NH1 | ARG | B | 310 | 60.038 | 98.258 | 92.349 | 1.00 | 42.04 |
| ATOM | 8287 | NH2 | ARG | B | 310 | 61.298 | 96.422 | 92.870 | 1.00 | 41.05 |
| ATOM | 8288 | N | ILE | B | 311 | 56.158 | 92.800 | 88.453 | 1.00 | 33.58 |
| ATOM | 8289 | CA | ILE | B | 311 | 56.441 | 91.777 | 87.475 | 1.00 | 33.34 |
| ATOM | 8290 | C | ILE | B | 311 | 57.663 | 92.159 | 86.679 | 1.00 | 32.04 |
| ATOM | 8291 | O | ILE | B | 311 | 57.848 | 93.319 | 86.298 | 1.00 | 31.10 |
| ATOM | 8292 | CB | ILE | B | 311 | 55.290 | 91.632 | 86.488 | 1.00 | 33.75 |
| ATOM | 8293 | CG1 | ILE | B | 311 | 53.962 | 91.831 | 87.171 | 1.00 | 36.32 |
| ATOM | 8294 | CG2 | ILE | B | 311 | 55.320 | 90.264 | 85.828 | 1.00 | 33.59 |
| ATOM | 8295 | CD1 | ILE | B | 311 | 52.812 | 91.743 | 86.200 | 1.00 | 38.06 |
| ATOM | 8296 | N | SER | B | 312 | 58.482 | 91.162 | 86.401 | 1.00 | 30.98 |
| ATOM | 8297 | CA | SER | B | 312 | 59.650 | 91.365 | 85.576 | 1.00 | 30.92 |
| ATOM | 8298 | C | SER | B | 312 | 59.436 | 90.700 | 84.218 | 1.00 | 30.94 |
| ATOM | 8299 | O | SER | B | 312 | 59.183 | 89.489 | 84.163 | 1.00 | 31.01 |
| ATOM | 8300 | CB | SER | B | 312 | 60.824 | 90.703 | 86.242 | 1.00 | 30.36 |
| ATOM | 8301 | OG | SER | B | 312 | 61.950 | 91.004 | 85.505 | 1.00 | 28.13 |
| ATOM | 8302 | N | LEU | B | 313 | 59.540 | 91.468 | 83.139 | 1.00 | 31.72 |
| ATOM | 8303 | CA | LEU | B | 313 | 59.361 | 90.929 | 81.780 | 1.00 | 31.65 |
| ATOM | 8304 | C | LEU | B | 313 | 60.658 | 91.067 | 81.018 | 1.00 | 31.48 |
| ATOM | 8305 | O | LEU | B | 313 | 61.245 | 92.154 | 80.953 | 1.00 | 31.36 |
| ATOM | 8306 | CB | LEU | B | 313 | 58.273 | 91.687 | 81.038 | 1.00 | 32.02 |
| ATOM | 8307 | CG | LEU | B | 313 | 56.897 | 91.696 | 81.697 | 1.00 | 33.58 |
| ATOM | 8308 | CD1 | LEU | B | 313 | 56.013 | 92.618 | 80.928 | 1.00 | 33.88 |
| ATOM | 8309 | CD2 | LEU | B | 313 | 56.267 | 90.298 | 81.757 | 1.00 | 35.31 |
| ATOM | 8310 | N | GLN | B | 314 | 61.129 | 89.979 | 80.454 | 1.00 | 30.90 |
| ATOM | 8311 | CA | GLN | B | 314 | 62.364 | 90.044 | 79.708 | 1.00 | 31.97 |
| ATOM | 8312 | C | GLN | B | 314 | 62.066 | 89.884 | 78.215 | 1.00 | 31.49 |
| ATOM | 8313 | O | GLN | B | 314 | 61.493 | 88.884 | 77.814 | 1.00 | 32.91 |
| ATOM | 8314 | CB | GLN | B | 314 | 63.364 | 89.018 | 80.210 | 1.00 | 31.34 |
| ATOM | 8315 | CG | GLN | B | 314 | 64.795 | 89.386 | 79.861 | 1.00 | 35.07 |
| ATOM | 8316 | CD | GLN | B | 314 | 65.863 | 88.543 | 80.626 | 1.00 | 35.88 |
| ATOM | 8317 | OE1 | GLN | B | 314 | 67.037 | 88.877 | 80.607 | 1.00 | 37.83 |
| ATOM | 8318 | NE2 | GLN | B | 314 | 65.443 | 87.481 | 81.274 | 1.00 | 36.84 |
| ATOM | 8319 | N | TRP | B | 315 | 62.408 | 90.904 | 77.437 | 1.00 | 30.58 |
| ATOM | 8320 | CA | TRP | B | 315 | 62.148 | 90.949 | 76.010 | 1.00 | 30.91 |
| ATOM | 8321 | C | TRP | B | 315 | 63.425 | 90.754 | 75.234 | 1.00 | 29.95 |
| ATOM | 8322 | O | TRP | B | 315 | 64.484 | 91.034 | 75.744 | 1.00 | 31.13 |
| ATOM | 8323 | CB | TRP | B | 315 | 61.521 | 92.302 | 75.597 | 1.00 | 30.37 |
| ATOM | 8324 | CG | TRP | B | 315 | 60.236 | 92.639 | 76.303 | 1.00 | 31.01 |
| ATOM | 8325 | CD1 | TRP | B | 315 | 60.102 | 93.311 | 77.494 | 1.00 | 32.19 |
| ATOM | 8326 | CD2 | TRP | B | 315 | 58.904 | 92.327 | 75.881 | 1.00 | 30.33 |
| ATOM | 8327 | NE1 | TRP | B | 315 | 58.777 | 93.424 | 77.820 | 1.00 | 30.77 |
| ATOM | 8328 | CE2 | TRP | B | 315 | 58.025 | 92.848 | 76.839 | 1.00 | 29.77 |
| ATOM | 8329 | CE3 | TRP | B | 315 | 58.365 | 91.655 | 74.776 | 1.00 | 31.81 |
| ATOM | 8330 | CZ2 | TRP | B | 315 | 56.646 | 92.716 | 76.741 | 1.00 | 32.04 |
| ATOM | 8331 | CZ3 | TRP | B | 315 | 57.014 | 91.527 | 74.681 | 1.00 | 31.97 |
| ATOM | 8332 | CH2 | TRP | B | 315 | 56.162 | 92.050 | 75.655 | 1.00 | 31.73 |
| ATOM | 8333 | N | LEU | B | 316 | 63.319 | 90.296 | 73.996 | 1.00 | 29.83 |
| ATOM | 8334 | CA | LEU | B | 316 | 64.495 | 90.080 | 73.131 | 1.00 | 31.28 |
| ATOM | 8335 | C | LEU | B | 316 | 64.189 | 90.512 | 71.687 | 1.00 | 31.65 |
| ATOM | 8336 | O | LEU | B | 316 | 63.095 | 90.273 | 71.174 | 1.00 | 31.45 |
| ATOM | 8337 | CB | LEU | B | 316 | 64.932 | 88.610 | 73.164 | 1.00 | 30.42 |
| ATOM | 8338 | CG | LEU | B | 316 | 66.108 | 88.062 | 72.350 | 1.00 | 33.06 |
| ATOM | 8339 | CD1 | LEU | B | 316 | 67.422 | 88.670 | 72.724 | 1.00 | 34.47 |
| ATOM | 8340 | CD2 | LEU | B | 316 | 66.158 | 86.509 | 72.506 | 1.00 | 34.10 |
| ATOM | 8341 | N | ARG | B | 317 | 65.147 | 91.171 | 71.047 | 1.00 | 33.19 |
| ATOM | 8342 | CA | ARG | B | 317 | 64.979 | 91.553 | 69.659 | 1.00 | 34.37 |
| ATOM | 8343 | C | ARG | B | 317 | 65.123 | 90.335 | 68.784 | 1.00 | 33.87 |
| ATOM | 8344 | O | ARG | B | 317 | 65.815 | 89.380 | 69.140 | 1.00 | 34.03 |
| ATOM | 8345 | CB | ARG | B | 317 | 65.996 | 92.625 | 69.277 | 1.00 | 36.07 |
| ATOM | 8346 | CG | ARG | B | 317 | 65.670 | 94.068 | 69.853 | 1.00 | 37.65 |
| ATOM | 8347 | CD | ARG | B | 317 | 66.288 | 95.194 | 68.990 | 1.00 | 38.57 |
| ATOM | 8348 | NE | ARG | B | 317 | 66.022 | 96.529 | 69.532 | 1.00 | 40.43 |
| ATOM | 8349 | CZ | ARG | B | 317 | 66.934 | 97.509 | 69.653 | 1.00 | 40.38 |
| ATOM | 8350 | NH1 | ARG | B | 317 | 68.199 | 97.337 | 69.241 | 1.00 | 37.57 |
| ATOM | 8351 | NH2 | ARG | B | 317 | 66.568 | 98.677 | 70.180 | 1.00 | 37.66 |
| ATOM | 8352 | N | ARG | B | 318 | 64.436 | 90.339 | 67.652 | 1.00 | 33.75 |
| ATOM | 8353 | CA | ARG | B | 318 | 64.582 | 89.259 | 66.696 | 1.00 | 33.76 |
| ATOM | 8354 | C | ARG | B | 318 | 66.031 | 89.091 | 66.348 | 1.00 | 34.27 |
| ATOM | 8355 | O | ARG | B | 318 | 66.533 | 87.974 | 66.209 | 1.00 | 35.49 |
| ATOM | 8356 | CB | ARG | B | 318 | 63.749 | 89.501 | 65.431 | 1.00 | 33.09 |
| ATOM | 8357 | CG | ARG | B | 318 | 63.566 | 88.230 | 64.624 | 1.00 | 32.42 |
| ATOM | 8358 | CD | ARG | B | 318 | 62.759 | 88.436 | 63.348 | 1.00 | 32.20 |
| ATOM | 8359 | NE | ARG | B | 318 | 62.754 | 87.234 | 62.545 | 1.00 | 31.52 |
| ATOM | 8360 | CZ | ARG | B | 318 | 61.754 | 86.850 | 61.757 | 1.00 | 26.85 |
| ATOM | 8361 | NH1 | ARG | B | 318 | 60.670 | 87.581 | 61.616 | 1.00 | 25.05 |
| ATOM | 8362 | NH2 | ARG | B | 318 | 61.859 | 85.718 | 61.113 | 1.00 | 27.58 |
| ATOM | 8363 | N | ILE | B | 319 | 66.721 | 90.187 | 66.138 | 1.00 | 35.43 |
| ATOM | 8364 | CA | ILE | B | 319 | 68.178 | 90.117 | 66.032 | 1.00 | 36.02 |
| ATOM | 8365 | C | ILE | B | 319 | 68.609 | 89.953 | 67.496 | 1.00 | 35.99 |
| ATOM | 8366 | O | ILE | B | 319 | 68.636 | 90.912 | 68.248 | 1.00 | 36.06 |
| ATOM | 8367 | CB | ILE | B | 319 | 68.699 | 91.387 | 65.382 | 1.00 | 36.52 |
| ATOM | 8368 | CG1 | ILE | B | 319 | 68.314 | 91.373 | 63.903 | 1.00 | 39.38 |
| ATOM | 8369 | CG2 | ILE | B | 319 | 70.197 | 91.477 | 65.464 | 1.00 | 38.69 |
| ATOM | 8370 | CD1 | ILE | B | 319 | 68.390 | 92.774 | 63.236 | 1.00 | 42.38 |
| ATOM | 8371 | N | GLN | B | 320 | 68.944 | 88.730 | 67.889 | 1.00 | 35.86 |
| ATOM | 8372 | CA | GLN | B | 320 | 69.067 | 88.366 | 69.300 | 1.00 | 36.22 |
| ATOM | 8373 | C | GLN | B | 320 | 70.372 | 88.820 | 69.962 | 1.00 | 36.74 |
| ATOM | 8374 | O | GLN | B | 320 | 71.063 | 88.031 | 70.624 | 1.00 | 36.55 |
| ATOM | 8375 | CB | GLN | B | 320 | 68.848 | 86.863 | 69.443 | 1.00 | 35.91 |
| ATOM | 8376 | CG | GLN | B | 320 | 67.536 | 86.390 | 68.779 | 1.00 | 36.08 |
| ATOM | 8377 | CD | GLN | B | 320 | 67.310 | 84.878 | 68.855 | 1.00 | 37.12 |
| ATOM | 8378 | OE1 | GLN | B | 320 | 67.791 | 84.200 | 69.778 | 1.00 | 33.28 |
| ATOM | 8379 | NE2 | GLN | B | 320 | 66.569 | 84.344 | 67.870 | 1.00 | 37.03 |
| ATOM | 8380 | N | ASN | B | 321 | 70.603 | 90.124 | 69.816 | 1.00 | 36.83 |
| ATOM | 8381 | CA | ASN | B | 321 | 71.791 | 90.866 | 70.229 | 1.00 | 37.99 |
| ATOM | 8382 | C | ASN | B | 321 | 71.561 | 91.766 | 71.422 | 1.00 | 36.96 |
| ATOM | 8383 | O | ASN | B | 321 | 72.496 | 92.360 | 71.978 | 1.00 | 36.20 |
| ATOM | 8384 | CB | ASN | B | 321 | 72.175 | 91.860 | 69.080 | 1.00 | 37.62 |
| ATOM | 8385 | CG | ASN | B | 321 | 73.398 | 91.459 | 68.385 | 1.00 | 41.47 |
| ATOM | 8386 | OD1 | ASN | B | 321 | 74.024 | 90.479 | 68.794 | 1.00 | 51.40 |
| ATOM | 8387 | ND2 | ASN | B | 321 | 73.790 | 92.188 | 67.323 | 1.00 | 45.34 |
| ATOM | 8388 | N | TYR | B | 322 | 70.294 | 91.917 | 71.760 | 1.00 | 37.02 |
| ATOM | 8389 | CA | TYR | B | 322 | 69.881 | 92.994 | 72.619 | 1.00 | 36.16 |
| ATOM | 8390 | C | TYR | B | 322 | 68.657 | 92.554 | 73.355 | 1.00 | 35.83 |
| ATOM | 8391 | O | TYR | B | 322 | 67.706 | 92.077 | 72.751 | 1.00 | 36.21 |
| ATOM | 8392 | CB | TYR | B | 322 | 69.586 | 94.207 | 71.703 | 1.00 | 36.28 |
| ATOM | 8393 | CG | TYR | B | 322 | 69.327 | 95.521 | 72.410 | 1.00 | 35.45 |
| ATOM | 8394 | CD1 | TYR | B | 322 | 70.357 | 96.402 | 72.714 | 1.00 | 35.79 |
| ATOM | 8395 | CD2 | TYR | B | 322 | 68.062 | 95.876 | 72.761 | 1.00 | 35.35 |
| ATOM | 8396 | CE1 | TYR | B | 322 | 70.102 | 97.614 | 73.393 | 1.00 | 35.68 |
| ATOM | 8397 | CE2 | TYR | B | 322 | 67.799 | 97.071 | 73.418 | 1.00 | 37.85 |
| ATOM | 8398 | CZ | TYR | B | 322 | 68.822 | 97.938 | 73.726 | 1.00 | 37.94 |
| ATOM | 8399 | OH | TYR | B | 322 | 68.509 | 99.110 | 74.380 | 1.00 | 39.12 |
| ATOM | 8400 | N | SER | B | 323 | 68.690 | 92.688 | 74.672 | 1.00 | 34.97 |
| ATOM | 8401 | CA | SER | B | 323 | 67.581 | 92.350 | 75.511 | 1.00 | 34.88 |
| ATOM | 8402 | C | SER | B | 323 | 67.423 | 93.386 | 76.600 | 1.00 | 34.54 |
| ATOM | 8403 | O | SER | B | 323 | 68.352 | 94.026 | 77.047 | 1.00 | 33.93 |
| ATOM | 8404 | CB | SER | B | 323 | 67.725 | 90.979 | 76.161 | 1.00 | 34.56 |
| ATOM | 8405 | OG | SER | B | 323 | 68.860 | 90.954 | 76.990 | 1.00 | 36.68 |
| ATOM | 8406 | N | VAL | B | 324 | 66.200 | 93.470 | 77.043 | 1.00 | 34.54 |
| ATOM | 8407 | CA | VAL | B | 324 | 65.787 | 94.427 | 77.996 | 1.00 | 34.86 |
| ATOM | 8408 | C | VAL | B | 324 | 64.873 | 93.729 | 78.955 | 1.00 | 35.00 |
| ATOM | 8409 | O | VAL | B | 324 | 63.910 | 93.100 | 78.542 | 1.00 | 35.60 |
| ATOM | 8410 | CB | VAL | B | 324 | 64.969 | 95.495 | 77.261 | 1.00 | 34.19 |
| ATOM | 8411 | CG1 | VAL | B | 324 | 64.281 | 96.374 | 78.220 | 1.00 | 34.10 |
| ATOM | 8412 | CG2 | VAL | B | 324 | 65.874 | 96.292 | 76.361 | 1.00 | 35.00 |
| ATOM | 8413 | N | MET | B | 325 | 65.165 | 93.885 | 80.226 | 1.00 | 35.13 |
| ATOM | 8414 | CA | MET | B | 325 | 64.326 | 93.430 | 81.296 | 1.00 | 36.34 |
| ATOM | 8415 | C | MET | B | 325 | 63.537 | 94.679 | 81.759 | 1.00 | 36.30 |
| ATOM | 8416 | O | MET | B | 325 | 64.130 | 95.719 | 82.062 | 1.00 | 35.13 |
| ATOM | 8417 | CB | MET | B | 325 | 65.227 | 92.906 | 82.409 | 1.00 | 37.09 |
| ATOM | 8418 | CG | MET | B | 325 | 64.619 | 91.939 | 83.381 | 1.00 | 40.89 |
| ATOM | 8419 | SD | MET | B | 325 | 65.964 | 91.400 | 84.519 | 1.00 | 44.02 |
| ATOM | 8420 | CE | MET | B | 325 | 65.131 | 91.287 | 85.946 | 1.00 | 41.92 |
| ATOM | 8421 | N | ASP | B | 326 | 62.206 | 94.579 | 81.759 | 1.00 | 36.01 |
| ATOM | 8422 | CA | ASP | B | 326 | 61.339 | 95.665 | 82.167 | 1.00 | 36.76 |
| ATOM | 8423 | C | ASP | B | 326 | 60.755 | 95.336 | 83.531 | 1.00 | 36.82 |
| ATOM | 8424 | O | ASP | B | 326 | 60.292 | 94.218 | 83.763 | 1.00 | 36.86 |
| ATOM | 8425 | CB | ASP | B | 326 | 60.186 | 95.785 | 81.196 | 1.00 | 37.27 |
| ATOM | 8426 | CG | ASP | B | 326 | 59.940 | 97.198 | 80.757 | 1.00 | 40.61 |
| ATOM | 8427 | OD1 | ASP | B | 326 | 60.662 | 98.086 | 81.246 | 1.00 | 43.19 |
| ATOM | 8428 | OD2 | ASP | B | 326 | 59.061 | 97.507 | 79.914 | 1.00 | 42.90 |
| ATOM | 8429 | N | ILE | B | 327 | 60.748 | 96.302 | 84.424 | 1.00 | 36.94 |
| ATOM | 8430 | CA | ILE | B | 327 | 60.206 | 96.073 | 85.740 | 1.00 | 37.80 |
| ATOM | 8431 | C | ILE | B | 327 | 58.933 | 96.886 | 85.884 | 1.00 | 38.50 |
| ATOM | 8432 | O | ILE | B | 327 | 58.924 | 98.104 | 85.704 | 1.00 | 38.44 |
| ATOM | 8433 | CB | ILE | B | 327 | 61.269 | 96.361 | 86.767 | 1.00 | 38.09 |
| ATOM | 8434 | CG1 | ILE | B | 327 | 62.305 | 95.246 | 86.660 | 1.00 | 38.18 |
| ATOM | 8435 | CG2 | ILE | B | 327 | 60.678 | 96.341 | 88.167 | 1.00 | 38.82 |
| ATOM | 8436 | CD1 | ILE | B | 327 | 63.637 | 95.704 | 86.910 | 1.00 | 41.48 |
| ATOM | 8437 | N | CYS | B | 328 | 57.843 | 96.182 | 86.157 | 1.00 | 39.11 |
| ATOM | 8438 | CA | CYS | B | 328 | 56.525 | 96.784 | 86.136 | 1.00 | 40.17 |
| ATOM | 8439 | C | CYS | B | 328 | 55.810 | 96.662 | 87.478 | 1.00 | 40.31 |
| ATOM | 8440 | O | CYS | B | 328 | 55.657 | 95.574 | 88.012 | 1.00 | 39.11 |
| ATOM | 8441 | CB | CYS | B | 328 | 55.695 | 96.150 | 85.032 | 1.00 | 39.94 |
| ATOM | 8442 | SG | CYS | B | 328 | 56.529 | 96.102 | 83.429 | 1.00 | 43.57 |
| ATOM | 8443 | N | ASP | B | 329 | 55.339 | 97.803 | 87.971 | 1.00 | 41.37 |
| ATOM | 8444 | CA | ASP | B | 329 | 54.739 | 97.898 | 89.300 | 1.00 | 42.47 |
| ATOM | 8445 | C | ASP | B | 329 | 53.266 | 98.195 | 89.268 | 1.00 | 43.58 |
| ATOM | 8446 | O | ASP | B | 329 | 52.818 | 99.060 | 88.512 | 1.00 | 43.63 |
| ATOM | 8447 | CB | ASP | B | 329 | 55.442 | 98.978 | 90.099 | 1.00 | 41.46 |
| ATOM | 8448 | CG | ASP | B | 329 | 56.810 | 98.539 | 90.582 | 1.00 | 43.42 |
| ATOM | 8449 | OD1 | ASP | B | 329 | 57.151 | 97.335 | 90.435 | 1.00 | 42.36 |
| ATOM | 8450 | OD2 | ASP | B | 329 | 57.622 | 99.334 | 91.110 | 1.00 | 45.24 |
| ATOM | 8451 | N | TYR | B | 330 | 52.509 | 97.463 | 90.068 | 1.00 | 45.51 |
| ATOM | 8452 | CA | TYR | B | 330 | 51.080 | 97.712 | 90.145 | 1.00 | 48.06 |
| ATOM | 8453 | C | TYR | B | 330 | 50.792 | 99.073 | 90.787 | 1.00 | 49.73 |
| ATOM | 8454 | O | TYR | B | 330 | 51.414 | 99.436 | 91.778 | 1.00 | 48.59 |
| ATOM | 8455 | CB | TYR | B | 330 | 50.361 | 96.626 | 90.924 | 1.00 | 48.00 |
| ATOM | 8456 | CG | TYR | B | 330 | 48.883 | 96.872 | 90.940 | 1.00 | 49.60 |
| ATOM | 8457 | CD1 | TYR | B | 330 | 48.186 | 97.056 | 89.750 | 1.00 | 52.40 |
| ATOM | 8458 | CD2 | TYR | B | 330 | 48.182 | 96.962 | 92.132 | 1.00 | 49.68 |
| ATOM | 8459 | CE1 | TYR | B | 330 | 46.829 | 97.307 | 89.752 | 1.00 | 51.02 |
| ATOM | 8460 | CE2 | TYR | B | 330 | 46.831 | 97.198 | 92.141 | 1.00 | 51.01 |
| ATOM | 8461 | CZ | TYR | B | 330 | 46.170 | 97.381 | 90.947 | 1.00 | 50.55 |
| ATOM | 8462 | OH | TYR | B | 330 | 44.838 | 97.637 | 90.945 | 1.00 | 52.45 |
| ATOM | 8463 | N | ASP | B | 331 | 49.863 | 99.826 | 90.199 | 1.00 | 52.21 |
| ATOM | 8464 | CA | ASP | B | 331 | 49.489 | 101.142 | 90.731 | 1.00 | 54.21 |
| ATOM | 8465 | C | ASP | B | 331 | 48.147 | 101.135 | 91.443 | 1.00 | 55.17 |
| ATOM | 8466 | O | ASP | B | 331 | 47.102 | 101.105 | 90.809 | 1.00 | 54.64 |
| ATOM | 8467 | CB | ASP | B | 331 | 49.425 | 102.188 | 89.616 | 1.00 | 54.82 |
| ATOM | 8468 | CG | ASP | B | 331 | 49.389 | 103.623 | 90.159 | 1.00 | 56.20 |
| ATOM | 8469 | OD1 | ASP | B | 331 | 49.417 | 103.797 | 91.402 | 1.00 | 57.58 |
| ATOM | 8470 | OD2 | ASP | B | 331 | 49.333 | 104.630 | 89.417 | 1.00 | 55.34 |
| ATOM | 8471 | N | GLU | B | 332 | 48.207 | 101.164 | 92.764 | 1.00 | 57.04 |
| ATOM | 8472 | CA | GLU | B | 332 | 47.033 | 101.259 | 93.634 | 1.00 | 58.48 |
| ATOM | 8473 | C | GLU | B | 332 | 45.973 | 102.249 | 93.098 | 1.00 | 58.60 |
| ATOM | 8474 | O | GLU | B | 332 | 44.781 | 101.938 | 93.040 | 1.00 | 58.62 |
| ATOM | 8475 | CB | GLU | B | 332 | 47.508 | 101.647 | 95.064 | 1.00 | 59.12 |
| ATOM | 8476 | CG | GLU | B | 332 | 47.186 | 103.075 | 95.550 | 1.00 | 61.50 |
| ATOM | 8477 | CD | GLU | B | 332 | 48.209 | 103.648 | 96.544 | 1.00 | 63.70 |
| ATOM | 8478 | OE1 | GLU | B | 332 | 48.425 | 103.040 | 97.621 | 1.00 | 63.97 |
| ATOM | 8479 | OE2 | GLU | B | 332 | 48.785 | 104.733 | 96.254 | 1.00 | 64.40 |
| ATOM | 8480 | N | SER | B | 333 | 46.427 | 103.424 | 92.677 | 1.00 | 59.26 |
| ATOM | 8481 | CA | SER | B | 333 | 45.549 | 104.481 | 92.182 | 1.00 | 59.33 |
| ATOM | 8482 | C | SER | B | 333 | 44.928 | 104.118 | 90.856 | 1.00 | 59.48 |
| ATOM | 8483 | O | SER | B | 333 | 43.722 | 103.946 | 90.745 | 1.00 | 60.00 |
| ATOM | 8484 | CB | SER | B | 333 | 46.337 | 105.789 | 91.992 | 1.00 | 59.31 |
| ATOM | 8485 | OG | SER | B | 333 | 46.801 | 106.327 | 93.225 | 1.00 | 59.60 |
| ATOM | 8486 | N | SER | B | 334 | 45.779 | 104.027 | 89.841 | 1.00 | 59.75 |
| ATOM | 8487 | CA | SER | B | 334 | 45.358 | 103.794 | 88.464 | 1.00 | 59.29 |
| ATOM | 8488 | C | SER | B | 334 | 44.758 | 102.424 | 88.202 | 1.00 | 58.62 |
| ATOM | 8489 | O | SER | B | 334 | 43.925 | 102.277 | 87.321 | 1.00 | 58.94 |
| ATOM | 8490 | CB | SER | B | 334 | 46.570 | 103.984 | 87.535 | 1.00 | 59.95 |
| ATOM | 8491 | OG | SER | B | 334 | 46.844 | 102.813 | 86.757 | 1.00 | 60.68 |
| ATOM | 8492 | N | GLY | B | 335 | 45.177 | 101.424 | 88.966 | 1.00 | 57.60 |
| ATOM | 8493 | CA | GLY | B | 335 | 44.808 | 100.056 | 88.676 | 1.00 | 56.75 |
| ATOM | 8494 | C | GLY | B | 335 | 45.530 | 99.590 | 87.413 | 1.00 | 55.98 |
| ATOM | 8495 | O | GLY | B | 335 | 45.144 | 98.612 | 86.786 | 1.00 | 55.77 |
| ATOM | 8496 | N | ARG | B | 336 | 46.559 | 100.312 | 87.001 | 1.00 | 55.26 |
| ATOM | 8497 | CA | ARG | B | 336 | 47.340 | 99.878 | 85.849 | 1.00 | 55.10 |
| ATOM | 8498 | C | ARG | B | 336 | 48.786 | 99.564 | 86.214 | 1.00 | 53.48 |
| ATOM | 8499 | O | ARG | B | 336 | 49.166 | 99.575 | 87.390 | 1.00 | 53.40 |
| ATOM | 8500 | CB | ARG | B | 336 | 47.220 | 100.861 | 84.686 | 1.00 | 55.82 |
| ATOM | 8501 | CG | ARG | B | 336 | 45.872 | 100.694 | 83.991 | 1.00 | 58.15 |
| ATOM | 8502 | CD | ARG | B | 336 | 45.589 | 101.645 | 82.855 | 1.00 | 62.20 |
| ATOM | 8503 | NE | ARG | B | 336 | 44.205 | 101.486 | 82.401 | 1.00 | 65.16 |
| ATOM | 8504 | CZ | ARG | B | 336 | 43.836 | 100.932 | 81.248 | 1.00 | 67.78 |
| ATOM | 8505 | NH1 | ARG | B | 336 | 44.748 | 100.482 | 80.388 | 1.00 | 67.72 |
| ATOM | 8506 | NH2 | ARG | B | 336 | 42.540 | 100.829 | 80.952 | 1.00 | 67.74 |
| ATOM | 8507 | N | TRP | B | 337 | 49.579 | 99.242 | 85.209 | 1.00 | 51.65 |
| ATOM | 8508 | CA | TRP | B | 337 | 50.931 | 98.806 | 85.452 | 1.00 | 50.02 |
| ATOM | 8509 | C | TRP | B | 337 | 51.925 | 99.718 | 84.765 | 1.00 | 50.23 |
| ATOM | 8510 | O | TRP | B | 337 | 51.857 | 99.882 | 83.556 | 1.00 | 50.77 |
| ATOM | 8511 | CB | TRP | B | 337 | 51.077 | 97.379 | 84.940 | 1.00 | 49.16 |
| ATOM | 8512 | CG | TRP | B | 337 | 50.349 | 96.355 | 85.753 | 1.00 | 44.51 |
| ATOM | 8513 | CD1 | TRP | B | 337 | 49.075 | 95.930 | 85.582 | 1.00 | 39.99 |
| ATOM | 8514 | CD2 | TRP | B | 337 | 50.869 | 95.618 | 86.868 | 1.00 | 39.72 |
| ATOM | 8515 | NE1 | TRP | B | 337 | 48.767 | 94.970 | 86.514 | 1.00 | 38.99 |
| ATOM | 8516 | CE2 | TRP | B | 337 | 49.860 | 94.757 | 87.310 | 1.00 | 39.18 |
| ATOM | 8517 | CE3 | TRP | B | 337 | 52.090 | 95.593 | 87.521 | 1.00 | 37.17 |
| ATOM | 8518 | CZ2 | TRP | B | 337 | 50.033 | 93.896 | 88.373 | 1.00 | 39.08 |
| ATOM | 8519 | CZ3 | TRP | B | 337 | 52.270 | 94.735 | 88.557 | 1.00 | 37.64 |
| ATOM | 8520 | CH2 | TRP | B | 337 | 51.247 | 93.898 | 88.983 | 1.00 | 38.93 |
| ATOM | 8521 | N | ASN | B | 338 | 52.842 | 100.302 | 85.541 | 1.00 | 49.98 |
| ATOM | 8522 | CA | ASN | B | 338 | 53.878 | 101.208 | 85.024 | 1.00 | 49.89 |
| ATOM | 8523 | C | ASN | B | 338 | 55.257 | 100.575 | 85.007 | 1.00 | 49.40 |
| ATOM | 8524 | O | ASN | B | 338 | 55.649 | 99.926 | 85.961 | 1.00 | 49.36 |
| ATOM | 8525 | CB | ASN | B | 338 | 53.944 | 102.484 | 85.865 | 1.00 | 49.80 |
| ATOM | 8526 | CG | ASN | B | 338 | 52.696 | 103.329 | 85.718 | 1.00 | 51.11 |
| ATOM | 8527 | OD1 | ASN | B | 338 | 52.312 | 103.707 | 84.587 | 1.00 | 50.56 |
| ATOM | 8528 | ND2 | ASN | B | 338 | 52.030 | 103.611 | 86.847 | 1.00 | 47.68 |
| ATOM | 8529 | N | CYS | B | 339 | 55.961 | 100.768 | 83.905 | 1.00 | 49.38 |
| ATOM | 8530 | CA | CYS | B | 339 | 57.289 | 100.212 | 83.685 | 1.00 | 49.73 |
| ATOM | 8531 | C | CYS | B | 339 | 58.205 | 101.408 | 83.455 | 1.00 | 50.11 |
| ATOM | 8532 | O | CYS | B | 339 | 58.305 | 101.916 | 82.340 | 1.00 | 50.56 |
| ATOM | 8533 | CB | CYS | B | 339 | 57.337 | 99.255 | 82.452 | 1.00 | 49.43 |
| ATOM | 8534 | SG | CYS | B | 339 | 56.155 | 97.857 | 82.365 | 1.00 | 47.42 |
| ATOM | 8535 | N | LEU | B | 340 | 58.848 | 101.876 | 84.520 | 1.00 | 50.25 |
| ATOM | 8536 | CA | LEU | B | 340 | 59.744 | 103.020 | 84.431 | 1.00 | 50.41 |
| ATOM | 8537 | C | LEU | B | 340 | 60.955 | 102.752 | 83.556 | 1.00 | 50.03 |
| ATOM | 8538 | O | LEU | B | 340 | 61.773 | 101.891 | 83.832 | 1.00 | 49.59 |
| ATOM | 8539 | CB | LEU | B | 340 | 60.216 | 103.430 | 85.825 | 1.00 | 51.05 |
| ATOM | 8540 | CG | LEU | B | 340 | 59.748 | 104.746 | 86.446 | 1.00 | 52.01 |
| ATOM | 8541 | CD1 | LEU | B | 340 | 58.249 | 104.940 | 86.344 | 1.00 | 54.40 |
| ATOM | 8542 | CD2 | LEU | B | 340 | 60.184 | 104.750 | 87.902 | 1.00 | 53.37 |
| ATOM | 8543 | N | VAL | B | 341 | 61.081 | 103.541 | 82.506 | 1.00 | 50.16 |
| ATOM | 8544 | CA | VAL | B | 341 | 62.188 | 103.400 | 81.585 | 1.00 | 50.23 |
| ATOM | 8545 | C | VAL | B | 341 | 63.565 | 103.419 | 82.240 | 1.00 | 49.64 |
| ATOM | 8546 | O | VAL | B | 341 | 64.471 | 102.692 | 81.817 | 1.00 | 49.50 |
| ATOM | 8547 | CB | VAL | B | 341 | 62.143 | 104.493 | 80.532 | 1.00 | 50.32 |
| ATOM | 8548 | CG1 | VAL | B | 341 | 63.501 | 104.623 | 79.847 | 1.00 | 50.95 |
| ATOM | 8549 | CG2 | VAL | B | 341 | 61.020 | 104.205 | 79.513 | 1.00 | 51.51 |
| ATOM | 8550 | N | ALA | B | 342 | 63.746 | 104.222 | 83.275 | 1.00 | 48.96 |
| ATOM | 8551 | CA | ALA | B | 342 | 65.083 | 104.348 | 83.826 | 1.00 | 48.51 |
| ATOM | 8552 | C | ALA | B | 342 | 65.420 | 103.128 | 84.638 | 1.00 | 47.95 |
| ATOM | 8553 | O | ALA | B | 342 | 66.568 | 102.921 | 85.011 | 1.00 | 48.49 |
| ATOM | 8554 | CB | ALA | B | 342 | 65.232 | 105.631 | 84.654 | 1.00 | 48.86 |
| ATOM | 8555 | N | ARG | B | 343 | 64.434 | 102.278 | 84.875 | 1.00 | 47.01 |
| ATOM | 8556 | CA | ARG | B | 343 | 64.668 | 101.083 | 85.687 | 1.00 | 46.47 |
| ATOM | 8557 | C | ARG | B | 343 | 64.755 | 99.795 | 84.816 | 1.00 | 45.21 |
| ATOM | 8558 | O | ARG | B | 343 | 64.712 | 98.683 | 85.302 | 1.00 | 44.07 |
| ATOM | 8559 | CB | ARG | B | 343 | 63.614 | 101.071 | 86.782 | 1.00 | 46.53 |
| ATOM | 8560 | CG | ARG | B | 343 | 63.260 | 99.809 | 87.425 | 1.00 | 47.76 |
| ATOM | 8561 | CD | ARG | B | 343 | 61.939 | 99.939 | 88.173 | 1.00 | 49.49 |
| ATOM | 8562 | NE | ARG | B | 343 | 62.057 | 100.749 | 89.374 | 1.00 | 51.13 |
| ATOM | 8563 | CZ | ARG | B | 343 | 61.039 | 101.378 | 89.974 | 1.00 | 53.68 |
| ATOM | 8564 | NH1 | ARG | B | 343 | 59.819 | 101.320 | 89.455 | 1.00 | 54.07 |
| ATOM | 8565 | NH2 | ARG | B | 343 | 61.241 | 102.078 | 91.093 | 1.00 | 52.56 |
| ATOM | 8566 | N | GLN | B | 344 | 64.920 | 99.987 | 83.517 | 1.00 | 44.33 |
| ATOM | 8567 | CA | GLN | B | 344 | 65.117 | 98.889 | 82.586 | 1.00 | 43.95 |
| ATOM | 8568 | C | GLN | B | 344 | 66.514 | 98.317 | 82.736 | 1.00 | 43.83 |
| ATOM | 8569 | O | GLN | B | 344 | 67.463 | 99.061 | 82.880 | 1.00 | 43.78 |
| ATOM | 8570 | CB | GLN | B | 344 | 64.986 | 99.396 | 81.122 | 1.00 | 43.32 |
| ATOM | 8571 | CG | GLN | B | 344 | 63.550 | 99.535 | 80.623 | 1.00 | 43.47 |
| ATOM | 8572 | CD | GLN | B | 344 | 63.419 | 100.271 | 79.278 | 1.00 | 41.94 |
| ATOM | 8573 | OE1 | GLN | B | 344 | 64.364 | 100.388 | 78.534 | 1.00 | 39.19 |
| ATOM | 8574 | NE2 | GLN | B | 344 | 62.230 | 100.727 | 78.979 | 1.00 | 43.55 |
| ATOM | 8575 | N | HIS | B | 345 | 66.664 | 97.003 | 82.683 | 1.00 | 44.19 |
| ATOM | 8576 | CA | HIS | B | 345 | 68.009 | 96.443 | 82.590 | 1.00 | 44.32 |
| ATOM | 8577 | C | HIS | B | 345 | 68.256 | 95.843 | 81.221 | 1.00 | 43.03 |
| ATOM | 8578 | O | HIS | B | 345 | 67.430 | 95.099 | 80.700 | 1.00 | 43.19 |
| ATOM | 8579 | CB | HIS | B | 345 | 68.320 | 95.537 | 83.760 | 1.00 | 44.46 |
| ATOM | 8580 | CG | HIS | B | 345 | 68.718 | 96.323 | 84.975 | 1.00 | 49.20 |
| ATOM | 8581 | ND1 | HIS | B | 345 | 67.873 | 96.529 | 86.048 | 1.00 | 52.36 |
| ATOM | 8582 | CD2 | HIS | B | 345 | 69.851 | 97.019 | 85.246 | 1.00 | 51.01 |
| ATOM | 8583 | CE1 | HIS | B | 345 | 68.486 | 97.279 | 86.948 | 1.00 | 54.64 |
| ATOM | 8584 | NE2 | HIS | B | 345 | 69.683 | 97.597 | 86.480 | 1.00 | 54.56 |
| ATOM | 8585 | N | ILE | B | 346 | 69.396 | 96.226 | 80.648 | 1.00 | 41.59 |
| ATOM | 8586 | CA | ILE | B | 346 | 69.757 | 95.956 | 79.267 | 1.00 | 41.02 |
| ATOM | 8587 | C | ILE | B | 346 | 70.982 | 95.099 | 79.127 | 1.00 | 40.37 |
| ATOM | 8588 | O | ILE | B | 346 | 71.973 | 95.327 | 79.790 | 1.00 | 40.23 |
| ATOM | 8589 | CB | ILE | B | 346 | 70.050 | 97.301 | 78.577 | 1.00 | 41.46 |
| ATOM | 8590 | CG1 | ILE | B | 346 | 68.815 | 98.189 | 78.604 | 1.00 | 42.09 |
| ATOM | 8591 | CG2 | ILE | B | 346 | 70.507 | 97.096 | 77.141 | 1.00 | 41.20 |
| ATOM | 8592 | CD1 | ILE | B | 346 | 69.057 | 99.578 | 78.042 | 1.00 | 43.90 |
| ATOM | 8593 | N | GLU | B | 347 | 70.910 | 94.080 | 78.292 | 1.00 | 40.45 |
| ATOM | 8594 | CA | GLU | B | 347 | 72.074 | 93.254 | 78.001 | 1.00 | 41.42 |
| ATOM | 8595 | C | GLU | B | 347 | 72.243 | 93.244 | 76.498 | 1.00 | 42.11 |
| ATOM | 8596 | O | GLU | B | 347 | 71.252 | 93.214 | 75.761 | 1.00 | 41.58 |
| ATOM | 8597 | CB | GLU | B | 347 | 71.909 | 91.820 | 78.463 | 1.00 | 41.54 |
| ATOM | 8598 | CG | GLU | B | 347 | 71.613 | 91.647 | 79.925 | 1.00 | 42.14 |
| ATOM | 8599 | CD | GLU | B | 347 | 71.340 | 90.199 | 80.249 | 1.00 | 42.92 |
| ATOM | 8600 | OE1 | GLU | B | 347 | 71.894 | 89.339 | 79.524 | 1.00 | 40.30 |
| ATOM | 8601 | OE2 | GLU | B | 347 | 70.611 | 89.932 | 81.245 | 1.00 | 43.22 |
| ATOM | 8602 | N | MET | B | 348 | 73.497 | 93.293 | 76.063 | 1.00 | 42.77 |
| ATOM | 8603 | CA | MET | B | 348 | 73.847 | 93.338 | 74.670 | 1.00 | 43.61 |
| ATOM | 8604 | C | MET | B | 348 | 75.009 | 92.412 | 74.520 | 1.00 | 43.46 |
| ATOM | 8605 | O | MET | B | 348 | 75.694 | 92.113 | 75.477 | 1.00 | 41.97 |
| ATOM | 8606 | CB | MET | B | 348 | 74.345 | 94.728 | 74.243 | 1.00 | 44.65 |
| ATOM | 8607 | CG | MET | B | 348 | 73.612 | 95.910 | 74.808 | 1.00 | 47.60 |
| ATOM | 8608 | SD | MET | B | 348 | 73.971 | 97.448 | 73.889 | 1.00 | 54.54 |
| ATOM | 8609 | CE | MET | B | 348 | 73.036 | 98.636 | 74.807 | 1.00 | 55.02 |
| ATOM | 8610 | N | SER | B | 349 | 75.240 | 91.944 | 73.312 | 1.00 | 44.14 |
| ATOM | 8611 | CA | SER | B | 349 | 76.439 | 91.164 | 73.078 | 1.00 | 45.08 |
| ATOM | 8612 | C | SER | B | 349 | 77.087 | 91.677 | 71.836 | 1.00 | 45.42 |
| ATOM | 8613 | O | SER | B | 349 | 76.417 | 92.017 | 70.862 | 1.00 | 45.19 |
| ATOM | 8614 | CB | SER | B | 349 | 76.131 | 89.677 | 72.911 | 1.00 | 45.44 |
| ATOM | 8615 | OG | SER | B | 349 | 77.328 | 88.900 | 72.852 | 1.00 | 45.77 |
| ATOM | 8616 | N | THR | B | 350 | 78.403 | 91.742 | 71.876 | 1.00 | 46.25 |
| ATOM | 8617 | CA | THR | B | 350 | 79.156 | 92.062 | 70.682 | 1.00 | 46.88 |
| ATOM | 8618 | C | THR | B | 350 | 79.737 | 90.764 | 70.158 | 1.00 | 46.10 |
| ATOM | 8619 | O | THR | B | 350 | 79.806 | 90.559 | 68.952 | 1.00 | 47.60 |
| ATOM | 8620 | CB | THR | B | 350 | 80.264 | 93.119 | 70.981 | 1.00 | 47.27 |
| ATOM | 8621 | OG1 | THR | B | 350 | 79.877 | 94.396 | 70.448 | 1.00 | 48.80 |
| ATOM | 8622 | CG2 | THR | B | 350 | 81.510 | 92.828 | 70.216 | 1.00 | 48.44 |
| ATOM | 8623 | N | THR | B | 351 | 80.103 | 89.867 | 71.065 | 1.00 | 44.83 |
| ATOM | 8624 | CA | THR | B | 351 | 80.770 | 88.623 | 70.704 | 1.00 | 44.31 |
| ATOM | 8625 | C | THR | B | 351 | 79.832 | 87.607 | 69.986 | 1.00 | 42.17 |
| ATOM | 8626 | O | THR | B | 351 | 80.258 | 86.796 | 69.166 | 1.00 | 41.47 |
| ATOM | 8627 | CB | THR | B | 351 | 81.387 | 88.060 | 72.004 | 1.00 | 45.00 |
| ATOM | 8628 | OG1 | THR | B | 351 | 82.318 | 89.023 | 72.529 | 1.00 | 48.79 |
| ATOM | 8629 | CG2 | THR | B | 351 | 82.282 | 86.861 | 71.767 | 1.00 | 46.07 |
| ATOM | 8630 | N | GLY | B | 352 | 78.542 | 87.671 | 70.267 | 1.00 | 39.36 |
| ATOM | 8631 | CA | GLY | B | 352 | 77.638 | 86.685 | 69.715 | 1.00 | 37.02 |
| ATOM | 8632 | C | GLY | B | 352 | 76.230 | 86.990 | 70.099 | 1.00 | 34.71 |
| ATOM | 8633 | O | GLY | B | 352 | 75.762 | 88.057 | 69.773 | 1.00 | 33.50 |
| ATOM | 8634 | N | TRP | B | 353 | 75.573 | 86.073 | 70.815 | 1.00 | 32.66 |
| ATOM | 8635 | CA | TRP | B | 353 | 74.176 | 86.260 | 71.159 | 1.00 | 31.41 |
| ATOM | 8636 | C | TRP | B | 353 | 73.994 | 86.437 | 72.646 | 1.00 | 31.25 |
| ATOM | 8637 | O | TRP | B | 353 | 74.958 | 86.404 | 73.415 | 1.00 | 31.98 |
| ATOM | 8638 | CB | TRP | B | 353 | 73.325 | 85.102 | 70.607 | 1.00 | 31.08 |
| ATOM | 8639 | CG | TRP | B | 353 | 73.819 | 83.802 | 71.120 | 1.00 | 28.71 |
| ATOM | 8640 | CD1 | TRP | B | 353 | 73.403 | 83.170 | 72.216 | 1.00 | 27.12 |
| ATOM | 8641 | CD2 | TRP | B | 353 | 74.843 | 83.009 | 70.563 | 1.00 | 25.31 |
| ATOM | 8642 | NE1 | TRP | B | 353 | 74.118 | 82.023 | 72.394 | 1.00 | 26.82 |
| ATOM | 8643 | CE2 | TRP | B | 353 | 74.998 | 81.898 | 71.373 | 1.00 | 25.30 |
| ATOM | 8644 | CE3 | TRP | B | 353 | 75.624 | 83.105 | 69.428 | 1.00 | 22.97 |
| ATOM | 8645 | CZ2 | TRP | B | 353 | 75.929 | 80.904 | 71.113 | 1.00 | 24.59 |
| ATOM | 8646 | CZ3 | TRP | B | 353 | 76.557 | 82.129 | 69.184 | 1.00 | 23.91 |
| ATOM | 8647 | CH2 | TRP | B | 353 | 76.694 | 81.039 | 70.020 | 1.00 | 20.31 |
| ATOM | 8648 | N | VAL | B | 354 | 72.771 | 86.682 | 73.074 | 1.00 | 31.63 |
| ATOM | 8649 | CA | VAL | B | 354 | 72.506 | 86.887 | 74.500 | 1.00 | 32.39 |
| ATOM | 8650 | C | VAL | B | 354 | 72.115 | 85.591 | 75.189 | 1.00 | 32.75 |
| ATOM | 8651 | O | VAL | B | 354 | 71.120 | 85.006 | 74.814 | 1.00 | 32.29 |
| ATOM | 8652 | CB | VAL | B | 354 | 71.338 | 87.819 | 74.701 | 1.00 | 32.04 |
| ATOM | 8653 | CG1 | VAL | B | 354 | 71.114 | 88.057 | 76.202 | 1.00 | 32.87 |
| ATOM | 8654 | CG2 | VAL | B | 354 | 71.593 | 89.112 | 73.948 | 1.00 | 33.99 |
| ATOM | 8655 | N | GLY | B | 355 | 72.887 | 85.167 | 76.189 | 1.00 | 31.57 |
| ATOM | 8656 | CA | GLY | B | 355 | 72.635 | 83.934 | 76.909 | 1.00 | 32.00 |
| ATOM | 8657 | C | GLY | B | 355 | 73.115 | 82.677 | 76.207 | 1.00 | 32.28 |
| ATOM | 8658 | O | GLY | B | 355 | 73.858 | 82.730 | 75.246 | 1.00 | 32.47 |
| ATOM | 8659 | N | ARG | B | 356 | 72.718 | 81.525 | 76.715 | 1.00 | 33.25 |
| ATOM | 8660 | CA | ARG | B | 356 | 73.065 | 80.266 | 76.074 | 1.00 | 34.63 |
| ATOM | 8661 | C | ARG | B | 356 | 72.014 | 79.978 | 75.026 | 1.00 | 34.42 |
| ATOM | 8662 | O | ARG | B | 356 | 72.204 | 80.281 | 73.864 | 1.00 | 35.19 |
| ATOM | 8663 | CB | ARG | B | 356 | 73.173 | 79.159 | 77.121 | 1.00 | 34.64 |
| ATOM | 8664 | CG | ARG | B | 356 | 74.475 | 79.292 | 77.933 | 1.00 | 35.41 |
| ATOM | 8665 | CD | ARG | B | 356 | 74.571 | 78.329 | 79.080 | 1.00 | 35.52 |
| ATOM | 8666 | NE | ARG | B | 356 | 75.793 | 78.531 | 79.846 | 1.00 | 38.21 |
| ATOM | 8667 | CZ | ARG | B | 356 | 75.894 | 78.449 | 81.200 | 1.00 | 39.82 |
| ATOM | 8668 | NH1 | ARG | B | 356 | 74.820 | 78.183 | 81.957 | 1.00 | 36.94 |
| ATOM | 8669 | NH2 | ARG | B | 356 | 77.087 | 78.598 | 81.775 | 1.00 | 37.65 |
| ATOM | 8670 | N | PHE | B | 357 | 70.866 | 79.483 | 75.443 | 1.00 | 35.26 |
| ATOM | 8671 | CA | PHE | B | 357 | 69.758 | 79.256 | 74.502 | 1.00 | 35.55 |
| ATOM | 8672 | C | PHE | B | 357 | 68.712 | 80.331 | 74.630 | 1.00 | 34.88 |
| ATOM | 8673 | O | PHE | B | 357 | 67.720 | 80.325 | 73.919 | 1.00 | 34.09 |
| ATOM | 8674 | CB | PHE | B | 357 | 69.126 | 77.893 | 74.766 | 1.00 | 35.51 |
| ATOM | 8675 | CG | PHE | B | 357 | 70.019 | 76.753 | 74.386 | 1.00 | 37.45 |
| ATOM | 8676 | CD1 | PHE | B | 357 | 70.316 | 76.521 | 73.067 | 1.00 | 35.48 |
| ATOM | 8677 | CD2 | PHE | B | 357 | 70.577 | 75.928 | 75.351 | 1.00 | 39.96 |
| ATOM | 8678 | CE1 | PHE | B | 357 | 71.138 | 75.472 | 72.688 | 1.00 | 37.76 |
| ATOM | 8679 | CE2 | PHE | B | 357 | 71.415 | 74.876 | 74.969 | 1.00 | 41.24 |
| ATOM | 8680 | CZ | PHE | B | 357 | 71.702 | 74.667 | 73.635 | 1.00 | 37.57 |
| ATOM | 8681 | N | ARG | B | 358 | 68.954 | 81.268 | 75.546 | 1.00 | 35.42 |
| ATOM | 8682 | CA | ARG | B | 358 | 68.012 | 82.347 | 75.833 | 1.00 | 35.39 |
| ATOM | 8683 | C | ARG | B | 358 | 68.648 | 83.253 | 76.857 | 1.00 | 34.33 |
| ATOM | 8684 | O | ARG | B | 358 | 69.666 | 82.926 | 77.425 | 1.00 | 34.66 |
| ATOM | 8685 | CB | ARG | B | 358 | 66.667 | 81.805 | 76.370 | 1.00 | 35.32 |
| ATOM | 8686 | CG | ARG | B | 358 | 66.731 | 81.096 | 77.770 | 1.00 | 38.01 |
| ATOM | 8687 | CD | ARG | B | 358 | 65.429 | 80.293 | 78.171 | 1.00 | 42.56 |
| ATOM | 8688 | NE | ARG | B | 358 | 65.604 | 78.889 | 77.792 | 1.00 | 49.10 |
| ATOM | 8689 | CZ | ARG | B | 358 | 64.871 | 78.203 | 76.909 | 1.00 | 51.71 |
| ATOM | 8690 | NH1 | ARG | B | 358 | 63.820 | 78.739 | 76.303 | 1.00 | 52.49 |
| ATOM | 8691 | NH2 | ARG | B | 358 | 65.190 | 76.939 | 76.663 | 1.00 | 51.90 |
| ATOM | 8692 | N | PRO | B | 359 | 68.079 | 84.425 | 77.048 | 1.00 | 32.96 |
| ATOM | 8693 | CA | PRO | B | 359 | 68.495 | 85.278 | 78.148 | 1.00 | 32.14 |
| ATOM | 8694 | C | PRO | B | 359 | 68.309 | 84.553 | 79.478 | 1.00 | 30.76 |
| ATOM | 8695 | O | PRO | B | 359 | 67.307 | 83.888 | 79.667 | 1.00 | 30.00 |
| ATOM | 8696 | CB | PRO | B | 359 | 67.540 | 86.463 | 78.014 | 1.00 | 32.50 |
| ATOM | 8697 | CG | PRO | B | 359 | 67.289 | 86.504 | 76.527 | 1.00 | 33.46 |
| ATOM | 8698 | CD | PRO | B | 359 | 67.029 | 85.049 | 76.211 | 1.00 | 33.28 |
| ATOM | 8699 | N | SER | B | 360 | 69.266 | 84.675 | 80.397 | 1.00 | 30.44 |
| ATOM | 8700 | CA | SER | B | 360 | 69.198 | 83.945 | 81.676 | 1.00 | 29.20 |
| ATOM | 8701 | C | SER | B | 360 | 68.112 | 84.453 | 82.602 | 1.00 | 28.79 |
| ATOM | 8702 | O | SER | B | 360 | 67.624 | 85.562 | 82.505 | 1.00 | 28.07 |
| ATOM | 8703 | CB | SER | B | 360 | 70.522 | 84.017 | 82.409 | 1.00 | 29.03 |
| ATOM | 8704 | OG | SER | B | 360 | 70.890 | 85.376 | 82.554 | 1.00 | 30.75 |
| ATOM | 8705 | N | GLU | B | 361 | 67.754 | 83.603 | 83.528 | 1.00 | 29.04 |
| ATOM | 8706 | CA | GLU | B | 361 | 66.686 | 83.869 | 84.431 | 1.00 | 30.28 |
| ATOM | 8707 | C | GLU | B | 361 | 67.151 | 84.828 | 85.541 | 1.00 | 29.46 |
| ATOM | 8708 | O | GLU | B | 361 | 68.238 | 84.630 | 86.091 | 1.00 | 28.02 |
| ATOM | 8709 | CB | GLU | B | 361 | 66.244 | 82.518 | 85.049 | 1.00 | 30.80 |
| ATOM | 8710 | CG | GLU | B | 361 | 64.975 | 82.620 | 85.892 | 1.00 | 35.11 |
| ATOM | 8711 | CD | GLU | B | 361 | 64.623 | 81.357 | 86.687 | 1.00 | 40.13 |
| ATOM | 8712 | OE1 | GLU | B | 361 | 65.395 | 80.387 | 86.648 | 1.00 | 43.02 |
| ATOM | 8713 | OE2 | GLU | B | 361 | 63.549 | 81.338 | 87.357 | 1.00 | 42.81 |
| ATOM | 8714 | N | PRO | B | 362 | 66.335 | 85.829 | 85.892 | 1.00 | 29.14 |
| ATOM | 8715 | CA | PRO | B | 362 | 66.613 | 86.687 | 87.066 | 1.00 | 28.78 |
| ATOM | 8716 | C | PRO | B | 362 | 66.064 | 86.097 | 88.366 | 1.00 | 28.53 |
| ATOM | 8717 | O | PRO | B | 362 | 64.914 | 85.729 | 88.357 | 1.00 | 28.21 |
| ATOM | 8718 | CB | PRO | B | 362 | 65.840 | 87.974 | 86.748 | 1.00 | 29.13 |
| ATOM | 8719 | CG | PRO | B | 362 | 64.578 | 87.491 | 85.880 | 1.00 | 28.93 |
| ATOM | 8720 | CD | PRO | B | 362 | 65.101 | 86.246 | 85.188 | 1.00 | 29.81 |
| ATOM | 8721 | N | HIS | B | 363 | 66.838 | 86.048 | 89.456 | 1.00 | 28.24 |
| ATOM | 8722 | CA | HIS | B | 363 | 66.357 | 85.516 | 90.729 | 1.00 | 28.25 |
| ATOM | 8723 | C | HIS | B | 363 | 66.263 | 86.681 | 91.748 | 1.00 | 28.40 |
| ATOM | 8724 | O | HIS | B | 363 | 67.276 | 87.210 | 92.185 | 1.00 | 27.96 |
| ATOM | 8725 | CB | HIS | B | 363 | 67.272 | 84.399 | 91.234 | 1.00 | 28.51 |
| ATOM | 8726 | CG | HIS | B | 363 | 67.297 | 83.182 | 90.360 | 1.00 | 28.22 |
| ATOM | 8727 | ND1 | HIS | B | 363 | 67.792 | 83.195 | 89.071 | 1.00 | 32.54 |
| ATOM | 8728 | CD2 | HIS | B | 363 | 66.889 | 81.913 | 90.588 | 1.00 | 27.37 |
| ATOM | 8729 | CE1 | HIS | B | 363 | 67.688 | 81.984 | 88.549 | 1.00 | 30.27 |
| ATOM | 8730 | NE2 | HIS | B | 363 | 67.142 | 81.191 | 89.452 | 1.00 | 29.70 |
| ATOM | 8731 | N | PHE | B | 364 | 65.043 | 87.058 | 92.125 | 1.00 | 28.36 |
| ATOM | 8732 | CA | PHE | B | 364 | 64.802 | 88.199 | 92.989 | 1.00 | 28.95 |
| ATOM | 8733 | C | PHE | B | 364 | 64.917 | 87.945 | 94.485 | 1.00 | 29.03 |
| ATOM | 8734 | O | PHE | B | 364 | 64.492 | 86.934 | 94.994 | 1.00 | 29.68 |
| ATOM | 8735 | CB | PHE | B | 364 | 63.419 | 88.788 | 92.725 | 1.00 | 28.09 |
| ATOM | 8736 | CG | PHE | B | 364 | 63.342 | 89.597 | 91.458 | 1.00 | 30.67 |
| ATOM | 8737 | CD1 | PHE | B | 364 | 63.142 | 88.993 | 90.238 | 1.00 | 28.84 |
| ATOM | 8738 | CD2 | PHE | B | 364 | 63.483 | 90.953 | 91.484 | 1.00 | 29.07 |
| ATOM | 8739 | CE1 | PHE | B | 364 | 63.050 | 89.732 | 89.082 | 1.00 | 29.83 |
| ATOM | 8740 | CE2 | PHE | B | 364 | 63.385 | 91.681 | 90.314 | 1.00 | 31.45 |
| ATOM | 8741 | CZ | PHE | B | 364 | 63.179 | 91.048 | 89.107 | 1.00 | 28.43 |
| ATOM | 8742 | N | THR | B | 365 | 65.498 | 88.889 | 95.180 | 1.00 | 30.02 |
| ATOM | 8743 | CA | THR | B | 365 | 65.501 | 88.851 | 96.639 | 1.00 | 31.47 |
| ATOM | 8744 | C | THR | B | 365 | 64.051 | 88.945 | 97.071 | 1.00 | 32.11 |
| ATOM | 8745 | O | THR | B | 365 | 63.203 | 89.406 | 96.319 | 1.00 | 32.79 |
| ATOM | 8746 | CB | THR | B | 365 | 66.180 | 90.081 | 97.159 | 1.00 | 31.27 |
| ATOM | 8747 | OG1 | THR | B | 365 | 65.607 | 91.202 | 96.482 | 1.00 | 28.45 |
| ATOM | 8748 | CG2 | THR | B | 365 | 67.593 | 90.095 | 96.767 | 1.00 | 33.17 |
| ATOM | 8749 | N | LEU | B | 366 | 63.778 | 88.542 | 98.288 | 1.00 | 33.65 |
| ATOM | 8750 | CA | LEU | B | 366 | 62.422 | 88.551 | 98.822 | 1.00 | 34.69 |
| ATOM | 8751 | C | LEU | B | 366 | 61.714 | 89.899 | 98.692 | 1.00 | 34.24 |
| ATOM | 8752 | O | LEU | B | 366 | 60.527 | 89.936 | 98.435 | 1.00 | 33.60 |
| ATOM | 8753 | CB | LEU | B | 366 | 62.437 | 88.096 | 100.286 | 1.00 | 35.14 |
| ATOM | 8754 | CG | LEU | B | 366 | 61.060 | 87.911 | 100.930 | 1.00 | 38.40 |
| ATOM | 8755 | CD1 | LEU | B | 366 | 60.213 | 86.873 | 100.145 | 1.00 | 39.24 |
| ATOM | 8756 | CD2 | LEU | B | 366 | 61.191 | 87.466 | 102.408 | 1.00 | 41.28 |
| ATOM | 8757 | N | ASP | B | 367 | 62.415 | 91.019 | 98.842 | 1.00 | 34.28 |
| ATOM | 8758 | CA | ASP | B | 367 | 61.708 | 92.303 | 98.718 | 1.00 | 34.37 |
| ATOM | 8759 | C | ASP | B | 367 | 61.542 | 92.770 | 97.272 | 1.00 | 33.70 |
| ATOM | 8760 | O | ASP | B | 367 | 60.904 | 93.778 | 97.025 | 1.00 | 34.18 |
| ATOM | 8761 | CB | ASP | B | 367 | 62.381 | 93.386 | 99.544 | 1.00 | 34.77 |
| ATOM | 8762 | CG | ASP | B | 367 | 63.844 | 93.626 | 99.136 | 1.00 | 38.12 |
| ATOM | 8763 | OD1 | ASP | B | 367 | 64.185 | 93.593 | 97.906 | 1.00 | 37.36 |
| ATOM | 8764 | OD2 | ASP | B | 367 | 64.721 | 93.849 | 100.003 | 1.00 | 40.82 |
| ATOM | 8765 | N | GLY | B | 368 | 62.096 | 92.040 | 96.315 | 1.00 | 33.10 |
| ATOM | 8766 | CA | GLY | B | 368 | 61.976 | 92.413 | 94.912 | 1.00 | 33.37 |
| ATOM | 8767 | C | GLY | B | 368 | 62.787 | 93.633 | 94.521 | 1.00 | 33.02 |
| ATOM | 8768 | O | GLY | B | 368 | 62.665 | 94.129 | 93.412 | 1.00 | 32.64 |
| ATOM | 8769 | N | ASN | B | 369 | 63.658 | 94.091 | 95.415 | 1.00 | 32.97 |
| ATOM | 8770 | CA | ASN | B | 369 | 64.459 | 95.265 | 95.121 | 1.00 | 32.76 |
| ATOM | 8771 | C | ASN | B | 369 | 65.768 | 94.991 | 94.482 | 1.00 | 30.68 |
| ATOM | 8772 | O | ASN | B | 369 | 66.471 | 95.905 | 94.109 | 1.00 | 29.88 |
| ATOM | 8773 | CB | ASN | B | 369 | 64.692 | 96.083 | 96.380 | 1.00 | 33.68 |
| ATOM | 8774 | CG | ASN | B | 369 | 63.565 | 96.993 | 96.653 | 1.00 | 36.77 |
| ATOM | 8775 | OD1 | ASN | B | 369 | 62.763 | 97.230 | 95.762 | 1.00 | 40.15 |
| ATOM | 8776 | ND2 | ASN | B | 369 | 63.460 | 97.500 | 97.902 | 1.00 | 44.89 |
| ATOM | 8777 | N | SER | B | 370 | 66.132 | 93.730 | 94.381 | 1.00 | 29.62 |
| ATOM | 8778 | CA | SER | B | 370 | 67.354 | 93.402 | 93.711 | 1.00 | 28.42 |
| ATOM | 8779 | C | SER | B | 370 | 67.210 | 91.984 | 93.213 | 1.00 | 27.80 |
| ATOM | 8780 | O | SER | B | 370 | 66.286 | 91.264 | 93.617 | 1.00 | 28.01 |
| ATOM | 8781 | CB | SER | B | 370 | 68.521 | 93.509 | 94.679 | 1.00 | 27.82 |
| ATOM | 8782 | OG | SER | B | 370 | 68.386 | 92.513 | 95.682 | 1.00 | 28.71 |
| ATOM | 8783 | N | PHE | B | 371 | 68.140 | 91.571 | 92.370 | 1.00 | 26.77 |
| ATOM | 8784 | CA | PHE | B | 371 | 68.122 | 90.218 | 91.872 | 1.00 | 26.51 |
| ATOM | 8785 | C | PHE | B | 371 | 69.476 | 89.724 | 91.429 | 1.00 | 25.96 |
| ATOM | 8786 | O | PHE | B | 371 | 70.433 | 90.494 | 91.261 | 1.00 | 26.04 |
| ATOM | 8787 | CB | PHE | B | 371 | 67.163 | 90.106 | 90.712 | 1.00 | 25.97 |
| ATOM | 8788 | CG | PHE | B | 371 | 67.525 | 90.965 | 89.516 | 1.00 | 26.30 |
| ATOM | 8789 | CD1 | PHE | B | 371 | 67.081 | 92.277 | 89.425 | 1.00 | 26.19 |
| ATOM | 8790 | CD2 | PHE | B | 371 | 68.305 | 90.470 | 88.493 | 1.00 | 26.20 |
| ATOM | 8791 | CE1 | PHE | B | 371 | 67.366 | 93.056 | 88.336 | 1.00 | 28.41 |
| ATOM | 8792 | CE2 | PHE | B | 371 | 68.589 | 91.241 | 87.380 | 1.00 | 25.01 |
| ATOM | 8793 | CZ | PHE | B | 371 | 68.126 | 92.543 | 87.299 | 1.00 | 26.85 |
| ATOM | 8794 | N | TYR | B | 372 | 69.560 | 88.422 | 91.244 | 1.00 | 25.84 |
| ATOM | 8795 | CA | TYR | B | 372 | 70.807 | 87.819 | 90.776 | 1.00 | 26.43 |
| ATOM | 8796 | C | TYR | B | 372 | 70.592 | 87.159 | 89.421 | 1.00 | 27.24 |
| ATOM | 8797 | O | TYR | B | 372 | 69.539 | 86.583 | 89.167 | 1.00 | 27.59 |
| ATOM | 8798 | CB | TYR | B | 372 | 71.299 | 86.800 | 91.791 | 1.00 | 26.09 |
| ATOM | 8799 | CG | TYR | B | 372 | 71.576 | 87.370 | 93.136 | 1.00 | 25.58 |
| ATOM | 8800 | CD1 | TYR | B | 372 | 70.561 | 87.609 | 94.014 | 1.00 | 25.70 |
| ATOM | 8801 | CD2 | TYR | B | 372 | 72.871 | 87.683 | 93.539 | 1.00 | 27.45 |
| ATOM | 8802 | CE1 | TYR | B | 372 | 70.812 | 88.117 | 95.313 | 1.00 | 25.19 |
| ATOM | 8803 | CE2 | TYR | B | 372 | 73.130 | 88.184 | 94.830 | 1.00 | 27.08 |
| ATOM | 8804 | CZ | TYR | B | 372 | 72.080 | 88.414 | 95.693 | 1.00 | 28.33 |
| ATOM | 8805 | OH | TYR | B | 372 | 72.281 | 88.915 | 96.950 | 1.00 | 28.13 |
| ATOM | 8806 | N | LYS | B | 373 | 71.611 | 87.194 | 88.572 | 1.00 | 27.72 |
| ATOM | 8807 | CA | LYS | B | 373 | 71.473 | 86.695 | 87.226 | 1.00 | 28.24 |
| ATOM | 8808 | C | LYS | B | 373 | 72.840 | 86.299 | 86.698 | 1.00 | 28.06 |
| ATOM | 8809 | O | LYS | B | 373 | 73.867 | 86.951 | 86.973 | 1.00 | 27.66 |
| ATOM | 8810 | CB | LYS | B | 373 | 70.877 | 87.824 | 86.341 | 1.00 | 28.35 |
| ATOM | 8811 | CG | LYS | B | 373 | 70.197 | 87.434 | 85.077 | 1.00 | 30.16 |
| ATOM | 8812 | CD | LYS | B | 373 | 69.538 | 88.673 | 84.427 | 1.00 | 31.31 |
| ATOM | 8813 | CE | LYS | B | 373 | 68.950 | 88.428 | 83.026 | 1.00 | 32.45 |
| ATOM | 8814 | NZ | LYS | B | 373 | 69.803 | 87.650 | 82.077 | 1.00 | 31.51 |
| ATOM | 8815 | N | ILE | B | 374 | 72.839 | 85.245 | 85.895 | 1.00 | 27.56 |
| ATOM | 8816 | CA | ILE | B | 374 | 74.059 | 84.780 | 85.249 | 1.00 | 27.63 |
| ATOM | 8817 | C | ILE | B | 374 | 74.259 | 85.606 | 83.996 | 1.00 | 27.86 |
| ATOM | 8818 | O | ILE | B | 374 | 73.350 | 85.803 | 83.226 | 1.00 | 26.36 |
| ATOM | 8819 | CB | ILE | B | 374 | 73.963 | 83.320 | 84.883 | 1.00 | 27.04 |
| ATOM | 8820 | CG1 | ILE | B | 374 | 73.842 | 82.470 | 86.148 | 1.00 | 27.49 |
| ATOM | 8821 | CG2 | ILE | B | 374 | 75.160 | 82.917 | 84.002 | 1.00 | 28.04 |
| ATOM | 8822 | CD1 | ILE | B | 374 | 73.590 | 81.009 | 85.875 | 1.00 | 27.19 |
| ATOM | 8823 | N | ILE | B | 375 | 75.478 | 86.053 | 83.817 | 1.00 | 29.57 |
| ATOM | 8824 | CA | ILE | B | 375 | 75.851 | 86.968 | 82.760 | 1.00 | 30.61 |
| ATOM | 8825 | C | ILE | B | 375 | 77.305 | 86.726 | 82.465 | 1.00 | 30.53 |
| ATOM | 8826 | O | ILE | B | 375 | 78.086 | 86.330 | 83.355 | 1.00 | 29.58 |
| ATOM | 8827 | CB | ILE | B | 375 | 75.602 | 88.438 | 83.230 | 1.00 | 31.78 |
| ATOM | 8828 | CG1 | ILE | B | 375 | 74.191 | 88.871 | 82.811 | 1.00 | 33.37 |
| ATOM | 8829 | CG2 | ILE | B | 375 | 76.591 | 89.407 | 82.605 | 1.00 | 31.79 |
| ATOM | 8830 | CD1 | ILE | B | 375 | 73.602 | 89.765 | 83.742 | 1.00 | 34.82 |
| ATOM | 8831 | N | SER | B | 376 | 77.646 | 86.930 | 81.202 | 1.00 | 30.08 |
| ATOM | 8832 | CA | SER | B | 376 | 78.992 | 86.734 | 80.730 | 1.00 | 30.69 |
| ATOM | 8833 | C | SER | B | 376 | 79.913 | 87.823 | 81.270 | 1.00 | 30.51 |
| ATOM | 8834 | O | SER | B | 376 | 79.617 | 89.003 | 81.142 | 1.00 | 29.37 |
| ATOM | 8835 | CB | SER | B | 376 | 78.973 | 86.729 | 79.200 | 1.00 | 30.58 |
| ATOM | 8836 | OG | SER | B | 376 | 80.189 | 86.251 | 78.714 | 1.00 | 32.76 |
| ATOM | 8837 | N | ASN | B | 377 | 81.026 | 87.440 | 81.884 | 1.00 | 31.48 |
| ATOM | 8838 | CA | ASN | B | 377 | 81.927 | 88.467 | 82.464 | 1.00 | 32.07 |
| ATOM | 8839 | C | ASN | B | 377 | 82.887 | 89.002 | 81.385 | 1.00 | 32.94 |
| ATOM | 8840 | O | ASN | B | 377 | 82.818 | 88.558 | 80.262 | 1.00 | 32.30 |
| ATOM | 8841 | CB | ASN | B | 377 | 82.631 | 87.984 | 83.751 | 1.00 | 30.65 |
| ATOM | 8842 | CG | ASN | B | 377 | 83.804 | 87.047 | 83.496 | 1.00 | 29.57 |
| ATOM | 8843 | OD1 | ASN | B | 377 | 84.375 | 86.530 | 84.451 | 1.00 | 29.05 |
| ATOM | 8844 | ND2 | ASN | B | 377 | 84.196 | 86.849 | 82.243 | 1.00 | 21.88 |
| ATOM | 8845 | N | GLU | B | 378 | 83.724 | 89.977 | 81.718 | 1.00 | 34.32 |
| ATOM | 8846 | CA | GLU | B | 378 | 84.634 | 90.586 | 80.743 | 1.00 | 35.84 |
| ATOM | 8847 | C | GLU | B | 378 | 85.590 | 89.559 | 80.127 | 1.00 | 35.27 |
| ATOM | 8848 | O | GLU | B | 378 | 86.042 | 89.730 | 79.014 | 1.00 | 34.81 |
| ATOM | 8849 | CB | GLU | B | 378 | 85.353 | 91.833 | 81.338 | 1.00 | 36.36 |
| ATOM | 8850 | CG | GLU | B | 378 | 84.435 | 93.079 | 81.291 | 1.00 | 40.98 |
| ATOM | 8851 | CD | GLU | B | 378 | 84.928 | 94.334 | 82.059 | 1.00 | 45.85 |
| ATOM | 8852 | OE1 | GLU | B | 378 | 86.144 | 94.551 | 82.247 | 1.00 | 48.23 |
| ATOM | 8853 | OE2 | GLU | B | 378 | 84.064 | 95.154 | 82.455 | 1.00 | 49.28 |
| ATOM | 8854 | N | GLU | B | 379 | 85.849 | 88.459 | 80.814 | 1.00 | 34.78 |
| ATOM | 8855 | CA | GLU | B | 379 | 86.670 | 87.420 | 80.221 | 1.00 | 35.19 |
| ATOM | 8856 | C | GLU | B | 379 | 85.865 | 86.476 | 79.313 | 1.00 | 34.29 |
| ATOM | 8857 | O | GLU | B | 379 | 86.427 | 85.632 | 78.660 | 1.00 | 34.07 |
| ATOM | 8858 | CB | GLU | B | 379 | 87.385 | 86.628 | 81.284 | 1.00 | 35.34 |
| ATOM | 8859 | CG | GLU | B | 379 | 88.484 | 87.413 | 81.956 | 1.00 | 39.50 |
| ATOM | 8860 | CD | GLU | B | 379 | 88.025 | 88.775 | 82.405 | 1.00 | 43.86 |
| ATOM | 8861 | OE1 | GLU | B | 379 | 87.059 | 88.832 | 83.215 | 1.00 | 48.30 |
| ATOM | 8862 | OE2 | GLU | B | 379 | 88.608 | 89.777 | 81.927 | 1.00 | 46.28 |
| ATOM | 8863 | N | GLY | B | 380 | 84.556 | 86.651 | 79.251 | 1.00 | 33.33 |
| ATOM | 8864 | CA | GLY | B | 380 | 83.705 | 85.747 | 78.507 | 1.00 | 32.76 |
| ATOM | 8865 | C | GLY | B | 380 | 83.334 | 84.470 | 79.280 | 1.00 | 31.95 |
| ATOM | 8866 | O | GLY | B | 380 | 83.083 | 83.450 | 78.654 | 1.00 | 32.76 |
| ATOM | 8867 | N | TYR | B | 381 | 83.316 | 84.498 | 80.613 | 1.00 | 29.75 |
| ATOM | 8868 | CA | TYR | B | 381 | 82.882 | 83.340 | 81.381 | 1.00 | 28.76 |
| ATOM | 8869 | C | TYR | B | 381 | 81.604 | 83.722 | 82.133 | 1.00 | 28.78 |
| ATOM | 8870 | O | TYR | B | 381 | 81.552 | 84.769 | 82.750 | 1.00 | 29.82 |
| ATOM | 8871 | CB | TYR | B | 381 | 83.947 | 82.866 | 82.363 | 1.00 | 28.72 |
| ATOM | 8872 | CG | TYR | B | 381 | 85.074 | 82.105 | 81.721 | 1.00 | 29.30 |
| ATOM | 8873 | CD1 | TYR | B | 381 | 86.142 | 82.766 | 81.160 | 1.00 | 31.49 |
| ATOM | 8874 | CD2 | TYR | B | 381 | 85.078 | 80.724 | 81.684 | 1.00 | 30.04 |
| ATOM | 8875 | CE1 | TYR | B | 381 | 87.193 | 82.072 | 80.532 | 1.00 | 31.47 |
| ATOM | 8876 | CE2 | TYR | B | 381 | 86.106 | 80.034 | 81.078 | 1.00 | 29.68 |
| ATOM | 8877 | CZ | TYR | B | 381 | 87.163 | 80.732 | 80.492 | 1.00 | 32.82 |
| ATOM | 8878 | OH | TYR | B | 381 | 88.216 | 80.059 | 79.885 | 1.00 | 36.53 |
| ATOM | 8879 | N | ARG | B | 382 | 80.580 | 82.874 | 82.059 | 1.00 | 27.19 |
| ATOM | 8880 | CA | ARG | B | 382 | 79.314 | 83.098 | 82.722 | 1.00 | 26.71 |
| ATOM | 8881 | C | ARG | B | 382 | 79.386 | 82.934 | 84.261 | 1.00 | 25.48 |
| ATOM | 8882 | O | ARG | B | 382 | 79.690 | 81.857 | 84.791 | 1.00 | 25.66 |
| ATOM | 8883 | CB | ARG | B | 382 | 78.277 | 82.151 | 82.102 | 1.00 | 27.16 |
| ATOM | 8884 | CG | ARG | B | 382 | 77.974 | 82.601 | 80.659 | 1.00 | 28.04 |
| ATOM | 8885 | CD | ARG | B | 382 | 77.363 | 81.557 | 79.695 | 1.00 | 30.58 |
| ATOM | 8886 | NE | ARG | B | 382 | 77.567 | 82.097 | 78.355 | 1.00 | 31.02 |
| ATOM | 8887 | CZ | ARG | B | 382 | 76.882 | 83.109 | 77.849 | 1.00 | 31.62 |
| ATOM | 8888 | NH1 | ARG | B | 382 | 75.855 | 83.620 | 78.497 | 1.00 | 31.41 |
| ATOM | 8889 | NH2 | ARG | B | 382 | 77.211 | 83.594 | 76.673 | 1.00 | 32.85 |
| ATOM | 8890 | N | HIS | B | 383 | 79.084 | 84.016 | 84.941 | 1.00 | 24.15 |
| ATOM | 8891 | CA | HIS | B | 383 | 79.165 | 84.137 | 86.401 | 1.00 | 23.61 |
| ATOM | 8892 | C | HIS | B | 383 | 77.978 | 84.910 | 86.989 | 1.00 | 23.83 |
| ATOM | 8893 | O | HIS | B | 383 | 77.300 | 85.645 | 86.276 | 1.00 | 22.88 |
| ATOM | 8894 | CB | HIS | B | 383 | 80.525 | 84.819 | 86.759 | 1.00 | 23.36 |
| ATOM | 8895 | CG | HIS | B | 383 | 81.656 | 83.841 | 86.857 | 1.00 | 23.03 |
| ATOM | 8896 | ND1 | HIS | B | 383 | 82.490 | 83.532 | 85.801 | 1.00 | 22.11 |
| ATOM | 8897 | CD2 | HIS | B | 383 | 82.079 | 83.088 | 87.893 | 1.00 | 25.36 |
| ATOM | 8898 | CE1 | HIS | B | 383 | 83.371 | 82.630 | 86.186 | 1.00 | 23.99 |
| ATOM | 8899 | NE2 | HIS | B | 383 | 83.139 | 82.340 | 87.454 | 1.00 | 25.80 |
| ATOM | 8900 | N | ILE | B | 384 | 77.739 | 84.763 | 88.302 | 1.00 | 25.28 |
| ATOM | 8901 | CA | ILE | B | 384 | 76.612 | 85.422 | 88.951 | 1.00 | 26.29 |
| ATOM | 8902 | C | ILE | B | 384 | 76.839 | 86.916 | 89.191 | 1.00 | 27.83 |
| ATOM | 8903 | O | ILE | B | 384 | 77.830 | 87.297 | 89.825 | 1.00 | 29.35 |
| ATOM | 8904 | CB | ILE | B | 384 | 76.295 | 84.719 | 90.285 | 1.00 | 26.60 |
| ATOM | 8905 | CG1 | ILE | B | 384 | 76.014 | 83.222 | 90.045 | 1.00 | 25.88 |
| ATOM | 8906 | CG2 | ILE | B | 384 | 75.167 | 85.373 | 90.954 | 1.00 | 25.18 |
| ATOM | 8907 | CD1 | ILE | B | 384 | 75.990 | 82.403 | 91.282 | 1.00 | 24.31 |
| ATOM | 8908 | N | CYS | B | 385 | 75.912 | 87.738 | 88.701 | 1.00 | 29.31 |
| ATOM | 8909 | CA | CYS | B | 385 | 75.907 | 89.180 | 88.884 | 1.00 | 31.49 |
| ATOM | 8910 | C | CYS | B | 385 | 74.746 | 89.630 | 89.761 | 1.00 | 30.90 |
| ATOM | 8911 | O | CYS | B | 385 | 73.610 | 89.178 | 89.583 | 1.00 | 29.44 |
| ATOM | 8912 | CB | CYS | B | 385 | 75.741 | 89.914 | 87.552 | 1.00 | 32.52 |
| ATOM | 8913 | SG | CYS | B | 385 | 77.023 | 91.169 | 87.259 | 1.00 | 41.33 |
| ATOM | 8914 | N | TYR | B | 386 | 75.048 | 90.574 | 90.659 | 1.00 | 30.34 |
| ATOM | 8915 | CA | TYR | B | 386 | 74.096 | 91.148 | 91.589 | 1.00 | 30.15 |
| ATOM | 8916 | C | TYR | B | 386 | 73.657 | 92.487 | 91.066 | 1.00 | 30.01 |
| ATOM | 8917 | O | TYR | B | 386 | 74.472 | 93.316 | 90.795 | 1.00 | 30.40 |
| ATOM | 8918 | CB | TYR | B | 386 | 74.762 | 91.325 | 92.964 | 1.00 | 30.21 |
| ATOM | 8919 | CG | TYR | B | 386 | 73.883 | 91.980 | 94.011 | 1.00 | 30.72 |
| ATOM | 8920 | CD1 | TYR | B | 386 | 72.621 | 91.474 | 94.301 | 1.00 | 29.66 |
| ATOM | 8921 | CD2 | TYR | B | 386 | 74.329 | 93.076 | 94.732 | 1.00 | 32.09 |
| ATOM | 8922 | CE1 | TYR | B | 386 | 71.802 | 92.084 | 95.267 | 1.00 | 33.09 |
| ATOM | 8923 | CE2 | TYR | B | 386 | 73.522 | 93.682 | 95.733 | 1.00 | 33.08 |
| ATOM | 8924 | CZ | TYR | B | 386 | 72.276 | 93.170 | 95.999 | 1.00 | 31.44 |
| ATOM | 8925 | OH | TYR | B | 386 | 71.473 | 93.751 | 96.949 | 1.00 | 36.22 |
| ATOM | 8926 | N | PHE | B | 387 | 72.359 | 92.689 | 90.939 | 1.00 | 30.76 |
| ATOM | 8927 | CA | PHE | B | 387 | 71.794 | 93.881 | 90.337 | 1.00 | 31.09 |
| ATOM | 8928 | C | PHE | B | 387 | 70.891 | 94.524 | 91.384 | 1.00 | 32.31 |
| ATOM | 8929 | O | PHE | B | 387 | 70.170 | 93.836 | 92.091 | 1.00 | 30.31 |
| ATOM | 8930 | CB | PHE | B | 387 | 70.906 | 93.514 | 89.127 | 1.00 | 31.28 |
| ATOM | 8931 | CG | PHE | B | 387 | 71.665 | 93.062 | 87.874 | 1.00 | 31.87 |
| ATOM | 8932 | CD1 | PHE | B | 387 | 71.999 | 91.738 | 87.675 | 1.00 | 30.46 |
| ATOM | 8933 | CD2 | PHE | B | 387 | 72.051 | 93.995 | 86.911 | 1.00 | 34.40 |
| ATOM | 8934 | CE1 | PHE | B | 387 | 72.683 | 91.335 | 86.539 | 1.00 | 33.89 |
| ATOM | 8935 | CE2 | PHE | B | 387 | 72.739 | 93.613 | 85.768 | 1.00 | 34.48 |
| ATOM | 8936 | CZ | PHE | B | 387 | 73.058 | 92.274 | 85.582 | 1.00 | 36.19 |
| ATOM | 8937 | N | GLN | B | 388 | 70.896 | 95.845 | 91.468 | 1.00 | 34.06 |
| ATOM | 8938 | CA | GLN | B | 388 | 69.942 | 96.521 | 92.340 | 1.00 | 35.90 |
| ATOM | 8939 | C | GLN | B | 388 | 69.069 | 97.263 | 91.372 | 1.00 | 37.74 |
| ATOM | 8940 | O | GLN | B | 388 | 69.591 | 97.877 | 90.453 | 1.00 | 37.76 |
| ATOM | 8941 | CB | GLN | B | 388 | 70.654 | 97.415 | 93.358 | 1.00 | 35.39 |
| ATOM | 8942 | CG | GLN | B | 388 | 71.594 | 96.584 | 94.276 | 1.00 | 36.10 |
| ATOM | 8943 | CD | GLN | B | 388 | 72.371 | 97.422 | 95.270 | 1.00 | 35.87 |
| ATOM | 8944 | OE1 | GLN | B | 388 | 72.861 | 98.479 | 94.926 | 1.00 | 38.43 |
| ATOM | 8945 | NE2 | GLN | B | 388 | 72.507 | 96.933 | 96.492 | 1.00 | 37.79 |
| ATOM | 8946 | N | ILE | B | 389 | 67.758 | 97.231 | 91.592 | 1.00 | 40.35 |
| ATOM | 8947 | CA | ILE | B | 389 | 66.774 | 97.704 | 90.603 | 1.00 | 43.25 |
| ATOM | 8948 | C | ILE | B | 389 | 66.861 | 99.086 | 89.988 | 1.00 | 45.07 |
| ATOM | 8949 | O | ILE | B | 389 | 66.611 | 99.222 | 88.787 | 1.00 | 47.31 |
| ATOM | 8950 | CB | ILE | B | 389 | 65.344 | 97.464 | 91.121 | 1.00 | 44.21 |
| ATOM | 8951 | CG1 | ILE | B | 389 | 64.826 | 96.163 | 90.527 | 1.00 | 45.14 |
| ATOM | 8952 | CG2 | ILE | B | 389 | 64.392 | 98.594 | 90.734 | 1.00 | 44.71 |
| ATOM | 8953 | CD1 | ILE | B | 389 | 63.959 | 95.453 | 91.453 | 1.00 | 47.44 |
| ATOM | 8954 | N | ASP | B | 390 | 67.181 | 100.110 | 90.755 | 1.00 | 46.92 |
| ATOM | 8955 | CA | ASP | B | 390 | 67.277 | 101.449 | 90.172 | 1.00 | 48.07 |
| ATOM | 8956 | C | ASP | B | 390 | 68.756 | 101.842 | 90.063 | 1.00 | 49.25 |
| ATOM | 8957 | O | ASP | B | 390 | 69.087 | 103.011 | 90.098 | 1.00 | 49.25 |
| ATOM | 8958 | CB | ASP | B | 390 | 66.516 | 102.482 | 91.045 | 1.00 | 48.49 |
| ATOM | 8959 | CG | ASP | B | 390 | 64.998 | 102.496 | 90.783 | 1.00 | 48.30 |
| ATOM | 8960 | OD1 | ASP | B | 390 | 64.590 | 102.514 | 89.612 | 1.00 | 49.59 |
| ATOM | 8961 | OD2 | ASP | B | 390 | 64.131 | 102.503 | 91.677 | 1.00 | 48.98 |
| ATOM | 8962 | N | LYS | B | 391 | 69.642 | 100.852 | 89.990 | 1.00 | 50.15 |
| ATOM | 8963 | CA | LYS | B | 391 | 71.075 | 101.102 | 89.821 | 1.00 | 50.80 |
| ATOM | 8964 | C | LYS | B | 391 | 71.482 | 100.336 | 88.592 | 1.00 | 50.82 |
| ATOM | 8965 | O | LYS | B | 391 | 70.958 | 99.261 | 88.320 | 1.00 | 51.41 |
| ATOM | 8966 | CB | LYS | B | 391 | 71.887 | 100.669 | 91.051 | 1.00 | 50.88 |
| ATOM | 8967 | CG | LYS | B | 391 | 71.596 | 101.499 | 92.317 | 1.00 | 51.89 |
| ATOM | 8968 | CD | LYS | B | 391 | 72.836 | 102.252 | 92.832 | 1.00 | 53.68 |
| ATOM | 8969 | CE | LYS | B | 391 | 72.503 | 103.218 | 93.957 | 1.00 | 55.27 |
| ATOM | 8970 | NZ | LYS | B | 391 | 73.674 | 104.105 | 94.333 | 1.00 | 57.04 |
| ATOM | 8971 | N | LYS | B | 392 | 72.448 | 100.865 | 87.867 | 1.00 | 51.42 |
| ATOM | 8972 | CA | LYS | B | 392 | 72.757 | 100.359 | 86.536 | 1.00 | 51.86 |
| ATOM | 8973 | C | LYS | B | 392 | 73.714 | 99.176 | 86.410 | 1.00 | 51.31 |
| ATOM | 8974 | O | LYS | B | 392 | 73.448 | 98.220 | 85.693 | 1.00 | 51.18 |
| ATOM | 8975 | CB | LYS | B | 392 | 73.261 | 101.506 | 85.648 | 1.00 | 52.67 |
| ATOM | 8976 | CG | LYS | B | 392 | 73.932 | 102.688 | 86.392 | 1.00 | 54.96 |
| ATOM | 8977 | CD | LYS | B | 392 | 75.348 | 102.953 | 85.871 | 1.00 | 58.49 |
| ATOM | 8978 | CE | LYS | B | 392 | 75.716 | 104.447 | 85.878 | 1.00 | 60.19 |
| ATOM | 8979 | NZ | LYS | B | 392 | 75.395 | 105.170 | 84.572 | 1.00 | 60.31 |
| ATOM | 8980 | N | ASP | B | 393 | 74.848 | 99.216 | 87.064 | 1.00 | 50.38 |
| ATOM | 8981 | CA | ASP | B | 393 | 75.774 | 98.139 | 86.809 | 1.00 | 50.08 |
| ATOM | 8982 | C | ASP | B | 393 | 75.479 | 96.973 | 87.709 | 1.00 | 48.21 |
| ATOM | 8983 | O | ASP | B | 393 | 74.579 | 97.024 | 88.532 | 1.00 | 49.43 |
| ATOM | 8984 | CB | ASP | B | 393 | 77.206 | 98.620 | 86.957 | 1.00 | 50.72 |
| ATOM | 8985 | CG | ASP | B | 393 | 77.617 | 99.543 | 85.820 | 1.00 | 53.43 |
| ATOM | 8986 | OD1 | ASP | B | 393 | 77.555 | 99.116 | 84.628 | 1.00 | 55.71 |
| ATOM | 8987 | OD2 | ASP | B | 393 | 77.999 | 100.714 | 86.034 | 1.00 | 57.39 |
| ATOM | 8988 | N | CYS | B | 394 | 76.187 | 95.884 | 87.536 | 1.00 | 45.33 |
| ATOM | 8989 | CA | CYS | B | 394 | 75.963 | 94.802 | 88.461 | 1.00 | 43.02 |
| ATOM | 8990 | C | CYS | B | 394 | 77.288 | 94.481 | 89.056 | 1.00 | 40.29 |
| ATOM | 8991 | O | CYS | B | 394 | 78.308 | 95.025 | 88.650 | 1.00 | 38.33 |
| ATOM | 8992 | CB | CYS | B | 394 | 75.347 | 93.604 | 87.766 | 1.00 | 43.53 |
| ATOM | 8993 | SG | CYS | B | 394 | 76.360 | 92.904 | 86.459 | 1.00 | 44.49 |
| ATOM | 8994 | N | THR | B | 395 | 77.250 | 93.629 | 90.060 | 1.00 | 37.49 |
| ATOM | 8995 | CA | THR | B | 395 | 78.437 | 93.246 | 90.763 | 1.00 | 35.54 |
| ATOM | 8996 | C | THR | B | 395 | 78.599 | 91.760 | 90.638 | 1.00 | 33.54 |
| ATOM | 8997 | O | THR | B | 395 | 77.741 | 90.995 | 91.057 | 1.00 | 32.26 |
| ATOM | 8998 | CB | THR | B | 395 | 78.290 | 93.644 | 92.268 | 1.00 | 35.46 |
| ATOM | 8999 | OG1 | THR | B | 395 | 78.242 | 95.060 | 92.361 | 1.00 | 34.51 |
| ATOM | 9000 | CG2 | THR | B | 395 | 79.534 | 93.247 | 93.102 | 1.00 | 35.50 |
| ATOM | 9001 | N | PHE | B | 396 | 79.705 | 91.356 | 90.053 | 1.00 | 32.02 |
| ATOM | 9002 | CA | PHE | B | 396 | 80.028 | 89.949 | 89.976 | 1.00 | 31.62 |
| ATOM | 9003 | C | PHE | B | 396 | 80.393 | 89.408 | 91.339 | 1.00 | 31.00 |
| ATOM | 9004 | O | PHE | B | 396 | 81.157 | 89.983 | 92.069 | 1.00 | 31.02 |
| ATOM | 9005 | CB | PHE | B | 396 | 81.158 | 89.704 | 88.994 | 1.00 | 31.73 |
| ATOM | 9006 | CG | PHE | B | 396 | 80.707 | 89.754 | 87.577 | 1.00 | 32.92 |
| ATOM | 9007 | CD1 | PHE | B | 396 | 79.940 | 88.718 | 87.049 | 1.00 | 32.79 |
| ATOM | 9008 | CD2 | PHE | B | 396 | 81.012 | 90.852 | 86.773 | 1.00 | 35.08 |
| ATOM | 9009 | CE1 | PHE | B | 396 | 79.491 | 88.766 | 85.732 | 1.00 | 31.84 |
| ATOM | 9010 | CE2 | PHE | B | 396 | 80.563 | 90.912 | 85.473 | 1.00 | 33.94 |
| ATOM | 9011 | CZ | PHE | B | 396 | 79.795 | 89.853 | 84.950 | 1.00 | 35.28 |
| ATOM | 9012 | N | ILE | B | 397 | 79.857 | 88.249 | 91.632 | 1.00 | 30.00 |
| ATOM | 9013 | CA | ILE | B | 397 | 79.987 | 87.618 | 92.911 | 1.00 | 28.98 |
| ATOM | 9014 | C | ILE | B | 397 | 80.719 | 86.311 | 92.760 | 1.00 | 28.29 |
| ATOM | 9015 | O | ILE | B | 397 | 81.147 | 85.707 | 93.756 | 1.00 | 26.77 |
| ATOM | 9016 | CB | ILE | B | 397 | 78.566 | 87.480 | 93.378 | 1.00 | 30.09 |
| ATOM | 9017 | CG1 | ILE | B | 397 | 78.255 | 88.618 | 94.308 | 1.00 | 30.11 |
| ATOM | 9018 | CG2 | ILE | B | 397 | 78.181 | 86.088 | 93.850 | 1.00 | 33.13 |
| ATOM | 9019 | CD1 | ILE | B | 397 | 77.145 | 89.355 | 93.736 | 1.00 | 34.11 |
| ATOM | 9020 | N | THR | B | 398 | 80.916 | 85.882 | 91.507 | 1.00 | 26.82 |
| ATOM | 9021 | CA | THR | B | 398 | 81.759 | 84.692 | 91.241 | 1.00 | 26.63 |
| ATOM | 9022 | C | THR | B | 398 | 82.607 | 85.058 | 90.082 | 1.00 | 26.53 |
| ATOM | 9023 | O | THR | B | 398 | 82.279 | 85.964 | 89.351 | 1.00 | 25.15 |
| ATOM | 9024 | CB | THR | B | 398 | 80.958 | 83.405 | 90.855 | 1.00 | 26.34 |
| ATOM | 9025 | OG1 | THR | B | 398 | 80.066 | 83.700 | 89.776 | 1.00 | 24.02 |
| ATOM | 9026 | CG2 | THR | B | 398 | 80.114 | 82.910 | 91.966 | 1.00 | 24.85 |
| ATOM | 9027 | N | LYS | B | 399 | 83.699 | 84.354 | 89.906 | 1.00 | 27.52 |
| ATOM | 9028 | CA | LYS | B | 399 | 84.562 | 84.650 | 88.796 | 1.00 | 29.60 |
| ATOM | 9029 | C | LYS | B | 399 | 85.525 | 83.540 | 88.621 | 1.00 | 28.96 |
| ATOM | 9030 | O | LYS | B | 399 | 85.620 | 82.659 | 89.454 | 1.00 | 27.40 |
| ATOM | 9031 | CB | LYS | B | 399 | 85.331 | 85.973 | 89.004 | 1.00 | 30.65 |
| ATOM | 9032 | CG | LYS | B | 399 | 86.196 | 85.968 | 90.241 | 1.00 | 33.69 |
| ATOM | 9033 | CD | LYS | B | 399 | 87.538 | 86.536 | 89.932 | 1.00 | 39.54 |
| ATOM | 9034 | CE | LYS | B | 399 | 87.562 | 88.053 | 89.776 | 1.00 | 41.78 |
| ATOM | 9035 | NZ | LYS | B | 399 | 88.942 | 88.504 | 89.306 | 1.00 | 45.54 |
| ATOM | 9036 | N | GLY | B | 400 | 86.219 | 83.587 | 87.494 | 1.00 | 28.97 |
| ATOM | 9037 | CA | GLY | B | 400 | 87.209 | 82.608 | 87.186 | 1.00 | 29.71 |
| ATOM | 9038 | C | GLY | B | 400 | 86.942 | 81.913 | 85.862 | 1.00 | 29.99 |
| ATOM | 9039 | O | GLY | B | 400 | 85.961 | 82.201 | 85.158 | 1.00 | 30.25 |
| ATOM | 9040 | N | THR | B | 401 | 87.839 | 81.000 | 85.550 | 1.00 | 29.97 |
| ATOM | 9041 | CA | THR | B | 401 | 87.836 | 80.234 | 84.296 | 1.00 | 31.55 |
| ATOM | 9042 | C | THR | B | 401 | 86.956 | 79.005 | 84.392 | 1.00 | 29.87 |
| ATOM | 9043 | O | THR | B | 401 | 87.419 | 77.881 | 84.282 | 1.00 | 30.82 |
| ATOM | 9044 | CB | THR | B | 401 | 89.266 | 79.725 | 84.038 | 1.00 | 31.58 |
| ATOM | 9045 | OG1 | THR | B | 401 | 90.167 | 80.834 | 83.969 | 1.00 | 35.93 |
| ATOM | 9046 | CG2 | THR | B | 401 | 89.362 | 79.179 | 82.687 | 1.00 | 35.92 |
| ATOM | 9047 | N | TRP | B | 402 | 85.684 | 79.219 | 84.592 | 1.00 | 28.95 |
| ATOM | 9048 | CA | TRP | B | 402 | 84.738 | 78.136 | 84.696 | 1.00 | 27.57 |
| ATOM | 9049 | C | TRP | B | 402 | 83.433 | 78.870 | 84.627 | 1.00 | 26.43 |
| ATOM | 9050 | O | TRP | B | 402 | 83.435 | 80.082 | 84.519 | 1.00 | 24.58 |
| ATOM | 9051 | CB | TRP | B | 402 | 84.908 | 77.356 | 85.991 | 1.00 | 27.62 |
| ATOM | 9052 | CG | TRP | B | 402 | 85.024 | 78.201 | 87.275 | 1.00 | 29.73 |
| ATOM | 9053 | CD1 | TRP | B | 402 | 86.192 | 78.594 | 87.908 | 1.00 | 31.52 |
| ATOM | 9054 | CD2 | TRP | B | 402 | 83.953 | 78.686 | 88.102 | 1.00 | 30.78 |
| ATOM | 9055 | NE1 | TRP | B | 402 | 85.906 | 79.278 | 89.065 | 1.00 | 31.48 |
| ATOM | 9056 | CE2 | TRP | B | 402 | 84.548 | 79.365 | 89.215 | 1.00 | 32.72 |
| ATOM | 9057 | CE3 | TRP | B | 402 | 82.564 | 78.599 | 88.039 | 1.00 | 28.88 |
| ATOM | 9058 | CZ2 | TRP | B | 402 | 83.797 | 79.979 | 90.216 | 1.00 | 30.83 |
| ATOM | 9059 | CZ3 | TRP | B | 402 | 81.810 | 79.196 | 89.044 | 1.00 | 30.58 |
| ATOM | 9060 | CH2 | TRP | B | 402 | 82.435 | 79.894 | 90.125 | 1.00 | 32.45 |
| ATOM | 9061 | N | GLU | B | 403 | 82.321 | 78.154 | 84.654 | 1.00 | 26.31 |
| ATOM | 9062 | CA | GLU | B | 403 | 81.032 | 78.798 | 84.531 | 1.00 | 25.43 |
| ATOM | 9063 | C | GLU | B | 403 | 79.993 | 78.330 | 85.506 | 1.00 | 24.97 |
| ATOM | 9064 | O | GLU | B | 403 | 79.951 | 77.164 | 85.890 | 1.00 | 24.08 |
| ATOM | 9065 | CB | GLU | B | 403 | 80.476 | 78.571 | 83.141 | 1.00 | 24.99 |
| ATOM | 9066 | CG | GLU | B | 403 | 81.355 | 79.047 | 82.020 | 1.00 | 26.01 |
| ATOM | 9067 | CD | GLU | B | 403 | 80.550 | 79.358 | 80.756 | 1.00 | 30.67 |
| ATOM | 9068 | OE1 | GLU | B | 403 | 79.631 | 78.581 | 80.456 | 1.00 | 33.68 |
| ATOM | 9069 | OE2 | GLU | B | 403 | 80.805 | 80.383 | 80.077 | 1.00 | 32.90 |
| ATOM | 9070 | N | VAL | B | 404 | 79.143 | 79.269 | 85.876 | 1.00 | 24.19 |
| ATOM | 9071 | CA | VAL | B | 404 | 78.021 | 78.964 | 86.702 | 1.00 | 25.23 |
| ATOM | 9072 | C | VAL | B | 404 | 77.000 | 78.456 | 85.747 | 1.00 | 25.03 |
| ATOM | 9073 | O | VAL | B | 404 | 76.678 | 79.074 | 84.712 | 1.00 | 23.31 |
| ATOM | 9074 | CB | VAL | B | 404 | 77.467 | 80.232 | 87.452 | 1.00 | 25.92 |
| ATOM | 9075 | CG1 | VAL | B | 404 | 76.106 | 79.958 | 88.025 | 1.00 | 27.06 |
| ATOM | 9076 | CG2 | VAL | B | 404 | 78.470 | 80.712 | 88.550 | 1.00 | 23.98 |
| ATOM | 9077 | N | ILE | B | 405 | 76.476 | 77.304 | 86.092 | 1.00 | 26.18 |
| ATOM | 9078 | CA | ILE | B | 405 | 75.489 | 76.685 | 85.257 | 1.00 | 26.99 |
| ATOM | 9079 | C | ILE | B | 405 | 74.079 | 77.139 | 85.605 | 1.00 | 26.62 |
| ATOM | 9080 | O | ILE | B | 405 | 73.263 | 77.366 | 84.720 | 1.00 | 25.39 |
| ATOM | 9081 | CB | ILE | B | 405 | 75.670 | 75.240 | 85.340 | 1.00 | 27.77 |
| ATOM | 9082 | CG1 | ILE | B | 405 | 76.971 | 74.952 | 84.604 | 1.00 | 30.83 |
| ATOM | 9083 | CG2 | ILE | B | 405 | 74.449 | 74.493 | 84.696 | 1.00 | 29.84 |
| ATOM | 9084 | CD1 | ILE | B | 405 | 77.439 | 73.644 | 84.822 | 1.00 | 33.92 |
| ATOM | 9085 | N | GLY | B | 406 | 73.803 | 77.312 | 86.884 | 1.00 | 25.76 |
| ATOM | 9086 | CA | GLY | B | 406 | 72.486 | 77.790 | 87.267 | 1.00 | 26.15 |
| ATOM | 9087 | C | GLY | B | 406 | 72.456 | 78.242 | 88.716 | 1.00 | 26.02 |
| ATOM | 9088 | O | GLY | B | 406 | 73.205 | 77.698 | 89.554 | 1.00 | 24.13 |
| ATOM | 9089 | N | ILE | B | 407 | 71.619 | 79.261 | 88.981 | 1.00 | 26.05 |
| ATOM | 9090 | CA | ILE | B | 407 | 71.280 | 79.674 | 90.347 | 1.00 | 26.77 |
| ATOM | 9091 | C | ILE | B | 407 | 70.081 | 78.868 | 90.832 | 1.00 | 27.33 |
| ATOM | 9092 | O | ILE | B | 407 | 68.983 | 78.933 | 90.237 | 1.00 | 27.55 |
| ATOM | 9093 | CB | ILE | B | 407 | 70.943 | 81.145 | 90.378 | 1.00 | 26.48 |
| ATOM | 9094 | CG1 | ILE | B | 407 | 72.205 | 81.953 | 90.101 | 1.00 | 26.38 |
| ATOM | 9095 | CG2 | ILE | B | 407 | 70.365 | 81.491 | 91.655 | 1.00 | 27.27 |
| ATOM | 9096 | CD1 | ILE | B | 407 | 71.902 | 83.371 | 89.644 | 1.00 | 25.94 |
| ATOM | 9097 | N | GLU | B | 408 | 70.277 | 78.133 | 91.923 | 1.00 | 26.92 |
| ATOM | 9098 | CA | GLU | B | 408 | 69.316 | 77.142 | 92.348 | 1.00 | 27.73 |
| ATOM | 9099 | C | GLU | B | 408 | 68.463 | 77.505 | 93.557 | 1.00 | 28.16 |
| ATOM | 9100 | O | GLU | B | 408 | 67.355 | 77.002 | 93.695 | 1.00 | 28.50 |
| ATOM | 9101 | CB | GLU | B | 408 | 70.033 | 75.846 | 92.615 | 1.00 | 28.28 |
| ATOM | 9102 | CG | GLU | B | 408 | 70.781 | 75.323 | 91.405 | 1.00 | 30.97 |
| ATOM | 9103 | CD | GLU | B | 408 | 69.860 | 74.929 | 90.275 | 1.00 | 31.77 |
| ATOM | 9104 | OE1 | GLU | B | 408 | 68.715 | 74.532 | 90.478 | 1.00 | 32.78 |
| ATOM | 9105 | OE2 | GLU | B | 408 | 70.269 | 75.048 | 89.156 | 1.00 | 39.65 |
| ATOM | 9106 | N | ALA | B | 409 | 68.983 | 78.347 | 94.438 | 1.00 | 27.97 |
| ATOM | 9107 | CA | ALA | B | 409 | 68.194 | 78.895 | 95.507 | 1.00 | 27.24 |
| ATOM | 9108 | C | ALA | B | 409 | 68.812 | 80.169 | 96.106 | 1.00 | 27.92 |
| ATOM | 9109 | O | ALA | B | 409 | 70.039 | 80.448 | 96.014 | 1.00 | 27.82 |
| ATOM | 9110 | CB | ALA | B | 409 | 67.968 | 77.835 | 96.584 | 1.00 | 28.64 |
| ATOM | 9111 | N | LEU | B | 410 | 67.951 | 80.934 | 96.753 | 1.00 | 28.55 |
| ATOM | 9112 | CA | LEU | B | 410 | 68.334 | 82.194 | 97.320 | 1.00 | 29.03 |
| ATOM | 9113 | C | LEU | B | 410 | 67.521 | 82.462 | 98.576 | 1.00 | 28.80 |
| ATOM | 9114 | O | LEU | B | 410 | 66.316 | 82.382 | 98.555 | 1.00 | 26.13 |
| ATOM | 9115 | CB | LEU | B | 410 | 68.073 | 83.295 | 96.278 | 1.00 | 29.70 |
| ATOM | 9116 | CG | LEU | B | 410 | 68.224 | 84.705 | 96.823 | 1.00 | 31.21 |
| ATOM | 9117 | CD1 | LEU | B | 410 | 69.711 | 85.058 | 96.967 | 1.00 | 32.59 |
| ATOM | 9118 | CD2 | LEU | B | 410 | 67.536 | 85.707 | 95.927 | 1.00 | 33.71 |
| ATOM | 9119 | N | THR | B | 411 | 68.229 | 82.682 | 99.680 | 1.00 | 29.36 |
| ATOM | 9120 | CA | THR | B | 411 | 67.656 | 83.132 | 100.933 | 1.00 | 30.44 |
| ATOM | 9121 | C | THR | B | 411 | 68.417 | 84.420 | 101.260 | 1.00 | 31.21 |
| ATOM | 9122 | O | THR | B | 411 | 69.276 | 84.849 | 100.517 | 1.00 | 31.07 |
| ATOM | 9123 | CB | THR | B | 411 | 67.882 | 82.113 | 102.087 | 1.00 | 30.75 |
| ATOM | 9124 | OG1 | THR | B | 411 | 69.276 | 82.093 | 102.466 | 1.00 | 30.99 |
| ATOM | 9125 | CG2 | THR | B | 411 | 67.613 | 80.696 | 101.653 | 1.00 | 31.01 |
| ATOM | 9126 | N | SER | B | 412 | 68.166 | 85.015 | 102.408 | 1.00 | 32.99 |
| ATOM | 9127 | CA | SER | B | 412 | 68.879 | 86.230 | 102.750 | 1.00 | 33.86 |
| ATOM | 9128 | C | SER | B | 412 | 70.348 | 86.027 | 103.168 | 1.00 | 33.76 |
| ATOM | 9129 | O | SER | B | 412 | 71.138 | 86.976 | 103.098 | 1.00 | 35.07 |
| ATOM | 9130 | CB | SER | B | 412 | 68.136 | 86.949 | 103.855 | 1.00 | 34.08 |
| ATOM | 9131 | OG | SER | B | 412 | 68.298 | 86.251 | 105.064 | 1.00 | 37.09 |
| ATOM | 9132 | N | ASP | B | 413 | 70.711 | 84.820 | 103.609 | 1.00 | 33.55 |
| ATOM | 9133 | CA | ASP | B | 413 | 72.094 | 84.541 | 104.037 | 1.00 | 33.66 |
| ATOM | 9134 | C | ASP | B | 413 | 72.955 | 83.923 | 102.951 | 1.00 | 33.40 |
| ATOM | 9135 | O | ASP | B | 413 | 74.177 | 84.069 | 102.965 | 1.00 | 34.09 |
| ATOM | 9136 | CB | ASP | B | 413 | 72.147 | 83.717 | 105.349 | 1.00 | 34.67 |
| ATOM | 9137 | CG | ASP | B | 413 | 71.291 | 84.350 | 106.472 | 1.00 | 37.43 |
| ATOM | 9138 | OD1 | ASP | B | 413 | 71.142 | 85.591 | 106.512 | 1.00 | 35.18 |
| ATOM | 9139 | OD2 | ASP | B | 413 | 70.682 | 83.677 | 107.314 | 1.00 | 41.17 |
| ATOM | 9140 | N | TYR | B | 414 | 72.315 | 83.241 | 102.004 | 1.00 | 32.74 |
| ATOM | 9141 | CA | TYR | B | 414 | 73.023 | 82.442 | 101.042 | 1.00 | 32.44 |
| ATOM | 9142 | C | TYR | B | 414 | 72.379 | 82.402 | 99.639 | 1.00 | 31.27 |
| ATOM | 9143 | O | TYR | B | 414 | 71.164 | 82.522 | 99.514 | 1.00 | 29.95 |
| ATOM | 9144 | CB | TYR | B | 414 | 73.061 | 81.010 | 101.548 | 1.00 | 33.10 |
| ATOM | 9145 | CG | TYR | B | 414 | 73.550 | 80.834 | 102.967 | 1.00 | 37.63 |
| ATOM | 9146 | CD1 | TYR | B | 414 | 74.871 | 81.095 | 103.311 | 1.00 | 43.56 |
| ATOM | 9147 | CD2 | TYR | B | 414 | 72.691 | 80.389 | 103.964 | 1.00 | 42.38 |
| ATOM | 9148 | CE1 | TYR | B | 414 | 75.319 | 80.908 | 104.606 | 1.00 | 45.87 |
| ATOM | 9149 | CE2 | TYR | B | 414 | 73.130 | 80.234 | 105.262 | 1.00 | 45.10 |
| ATOM | 9150 | CZ | TYR | B | 414 | 74.442 | 80.506 | 105.570 | 1.00 | 45.30 |
| ATOM | 9151 | OH | TYR | B | 414 | 74.876 | 80.344 | 106.861 | 1.00 | 51.32 |
| ATOM | 9152 | N | LEU | B | 415 | 73.237 | 82.251 | 98.615 | 1.00 | 29.76 |
| ATOM | 9153 | CA | LEU | B | 415 | 72.847 | 81.988 | 97.231 | 1.00 | 28.70 |
| ATOM | 9154 | C | LEU | B | 415 | 73.486 | 80.654 | 96.880 | 1.00 | 28.09 |
| ATOM | 9155 | O | LEU | B | 415 | 74.708 | 80.467 | 97.041 | 1.00 | 27.98 |
| ATOM | 9156 | CB | LEU | B | 415 | 73.371 | 83.056 | 96.271 | 1.00 | 28.13 |
| ATOM | 9157 | CG | LEU | B | 415 | 72.962 | 82.857 | 94.815 | 1.00 | 29.69 |
| ATOM | 9158 | CD1 | LEU | B | 415 | 72.755 | 84.190 | 94.108 | 1.00 | 29.73 |
| ATOM | 9159 | CD2 | LEU | B | 415 | 73.952 | 81.961 | 94.065 | 1.00 | 30.10 |
| ATOM | 9160 | N | TYR | B | 416 | 72.675 | 79.708 | 96.432 | 1.00 | 26.86 |
| ATOM | 9161 | CA | TYR | B | 416 | 73.186 | 78.391 | 96.098 | 1.00 | 26.62 |
| ATOM | 9162 | C | TYR | B | 416 | 73.166 | 78.226 | 94.565 | 1.00 | 25.78 |
| ATOM | 9163 | O | TYR | B | 416 | 72.160 | 78.527 | 93.927 | 1.00 | 26.80 |
| ATOM | 9164 | CB | TYR | B | 416 | 72.308 | 77.281 | 96.697 | 1.00 | 25.80 |
| ATOM | 9165 | CG | TYR | B | 416 | 72.240 | 77.193 | 98.173 | 1.00 | 28.15 |
| ATOM | 9166 | CD1 | TYR | B | 416 | 71.371 | 78.007 | 98.876 | 1.00 | 27.63 |
| ATOM | 9167 | CD2 | TYR | B | 416 | 72.953 | 76.256 | 98.868 | 1.00 | 28.81 |
| ATOM | 9168 | CE1 | TYR | B | 416 | 71.247 | 77.933 | 100.196 | 1.00 | 28.41 |
| ATOM | 9169 | CE2 | TYR | B | 416 | 72.822 | 76.176 | 100.242 | 1.00 | 31.88 |
| ATOM | 9170 | CZ | TYR | B | 416 | 71.970 | 77.042 | 100.887 | 1.00 | 30.74 |
| ATOM | 9171 | OH | TYR | B | 416 | 71.763 | 77.015 | 102.225 | 1.00 | 27.12 |
| ATOM | 9172 | N | TYR | B | 417 | 74.250 | 77.723 | 94.003 | 1.00 | 24.99 |
| ATOM | 9173 | CA | TYR | B | 417 | 74.373 | 77.573 | 92.573 | 1.00 | 25.31 |
| ATOM | 9174 | C | TYR | B | 417 | 75.128 | 76.325 | 92.181 | 1.00 | 24.04 |
| ATOM | 9175 | O | TYR | B | 417 | 75.848 | 75.757 | 92.985 | 1.00 | 23.73 |
| ATOM | 9176 | CB | TYR | B | 417 | 75.065 | 78.809 | 91.972 | 1.00 | 25.15 |
| ATOM | 9177 | CG | TYR | B | 417 | 76.537 | 78.928 | 92.293 | 1.00 | 28.08 |
| ATOM | 9178 | CD1 | TYR | B | 417 | 76.976 | 79.616 | 93.428 | 1.00 | 27.99 |
| ATOM | 9179 | CD2 | TYR | B | 417 | 77.500 | 78.329 | 91.491 | 1.00 | 28.55 |
| ATOM | 9180 | CE1 | TYR | B | 417 | 78.331 | 79.734 | 93.718 | 1.00 | 27.43 |
| ATOM | 9181 | CE2 | TYR | B | 417 | 78.852 | 78.418 | 91.811 | 1.00 | 29.29 |
| ATOM | 9182 | CZ | TYR | B | 417 | 79.253 | 79.143 | 92.915 | 1.00 | 28.63 |
| ATOM | 9183 | OH | TYR | B | 417 | 80.606 | 79.212 | 93.242 | 1.00 | 29.92 |
| ATOM | 9184 | N | ILE | B | 418 | 74.960 | 75.903 | 90.932 | 1.00 | 22.92 |
| ATOM | 9185 | CA | ILE | B | 418 | 75.687 | 74.766 | 90.395 | 1.00 | 22.89 |
| ATOM | 9186 | C | ILE | B | 418 | 76.734 | 75.268 | 89.427 | 1.00 | 22.06 |
| ATOM | 9187 | O | ILE | B | 418 | 76.488 | 76.179 | 88.668 | 1.00 | 21.82 |
| ATOM | 9188 | CB | ILE | B | 418 | 74.727 | 73.795 | 89.704 | 1.00 | 24.28 |
| ATOM | 9189 | CG1 | ILE | B | 418 | 73.965 | 73.011 | 90.762 | 1.00 | 26.51 |
| ATOM | 9190 | CG2 | ILE | B | 418 | 75.475 | 72.742 | 88.818 | 1.00 | 23.28 |
| ATOM | 9191 | CD1 | ILE | B | 418 | 72.754 | 72.473 | 90.209 | 1.00 | 26.35 |
| ATOM | 9192 | N | SER | B | 419 | 77.921 | 74.707 | 89.459 | 1.00 | 22.69 |
| ATOM | 9193 | CA | SER | B | 419 | 78.984 | 75.110 | 88.501 | 1.00 | 22.54 |
| ATOM | 9194 | C | SER | B | 419 | 79.863 | 73.981 | 88.105 | 1.00 | 22.43 |
| ATOM | 9195 | O | SER | B | 419 | 79.835 | 72.928 | 88.740 | 1.00 | 22.38 |
| ATOM | 9196 | CB | SER | B | 419 | 79.892 | 76.217 | 89.080 | 1.00 | 23.04 |
| ATOM | 9197 | OG | SER | B | 419 | 80.960 | 75.726 | 89.913 | 1.00 | 22.13 |
| ATOM | 9198 | N | ASN | B | 420 | 80.682 | 74.209 | 87.078 | 1.00 | 22.56 |
| ATOM | 9199 | CA | ASN | B | 420 | 81.700 | 73.236 | 86.735 | 1.00 | 23.33 |
| ATOM | 9200 | C | ASN | B | 420 | 83.064 | 73.570 | 87.316 | 1.00 | 24.36 |
| ATOM | 9201 | O | ASN | B | 420 | 84.080 | 73.076 | 86.795 | 1.00 | 23.91 |
| ATOM | 9202 | CB | ASN | B | 420 | 81.859 | 72.970 | 85.212 | 1.00 | 23.30 |
| ATOM | 9203 | CG | ASN | B | 420 | 82.003 | 74.234 | 84.387 | 1.00 | 23.65 |
| ATOM | 9204 | OD1 | ASN | B | 420 | 81.738 | 74.231 | 83.187 | 1.00 | 25.64 |
| ATOM | 9205 | ND2 | ASN | B | 420 | 82.358 | 75.321 | 85.026 | 1.00 | 24.56 |
| ATOM | 9206 | N | GLU | B | 421 | 83.104 | 74.307 | 88.432 | 1.00 | 25.65 |
| ATOM | 9207 | CA | GLU | B | 421 | 84.396 | 74.668 | 88.992 | 1.00 | 26.66 |
| ATOM | 9208 | C | GLU | B | 421 | 85.244 | 73.495 | 89.362 | 1.00 | 26.87 |
| ATOM | 9209 | O | GLU | B | 421 | 86.443 | 73.530 | 89.134 | 1.00 | 28.68 |
| ATOM | 9210 | CB | GLU | B | 421 | 84.304 | 75.596 | 90.251 | 1.00 | 27.29 |
| ATOM | 9211 | CG | GLU | B | 421 | 85.672 | 76.069 | 90.692 | 1.00 | 28.18 |
| ATOM | 9212 | CD | GLU | B | 421 | 85.657 | 77.091 | 91.819 | 1.00 | 30.20 |
| ATOM | 9213 | OE1 | GLU | B | 421 | 84.593 | 77.354 | 92.391 | 1.00 | 28.33 |
| ATOM | 9214 | OE2 | GLU | B | 421 | 86.743 | 77.642 | 92.101 | 1.00 | 30.05 |
| ATOM | 9215 | N | TYR | B | 422 | 84.668 | 72.470 | 89.966 | 1.00 | 28.24 |
| ATOM | 9216 | CA | TYR | B | 422 | 85.499 | 71.428 | 90.558 | 1.00 | 29.25 |
| ATOM | 9217 | C | TYR | B | 422 | 86.528 | 70.779 | 89.621 | 1.00 | 30.07 |
| ATOM | 9218 | O | TYR | B | 422 | 86.189 | 70.226 | 88.558 | 1.00 | 27.85 |
| ATOM | 9219 | CB | TYR | B | 422 | 84.630 | 70.374 | 91.187 | 1.00 | 29.87 |
| ATOM | 9220 | CG | TYR | B | 422 | 85.346 | 69.559 | 92.200 | 1.00 | 33.87 |
| ATOM | 9221 | CD1 | TYR | B | 422 | 86.083 | 70.174 | 93.237 | 1.00 | 35.94 |
| ATOM | 9222 | CD2 | TYR | B | 422 | 85.310 | 68.161 | 92.147 | 1.00 | 35.90 |
| ATOM | 9223 | CE1 | TYR | B | 422 | 86.749 | 69.415 | 94.164 | 1.00 | 35.72 |
| ATOM | 9224 | CE2 | TYR | B | 422 | 85.980 | 67.393 | 93.088 | 1.00 | 34.40 |
| ATOM | 9225 | CZ | TYR | B | 422 | 86.674 | 68.013 | 94.091 | 1.00 | 37.30 |
| ATOM | 9226 | OH | TYR | B | 422 | 87.336 | 67.228 | 95.014 | 1.00 | 39.63 |
| ATOM | 9227 | N | LYS | B | 423 | 87.788 | 70.862 | 90.058 | 1.00 | 30.25 |
| ATOM | 9228 | CA | LYS | B | 423 | 88.952 | 70.283 | 89.386 | 1.00 | 30.83 |
| ATOM | 9229 | C | LYS | B | 423 | 89.091 | 70.788 | 87.951 | 1.00 | 30.10 |
| ATOM | 9230 | O | LYS | B | 423 | 89.702 | 70.169 | 87.114 | 1.00 | 30.09 |
| ATOM | 9231 | CB | LYS | B | 423 | 88.912 | 68.756 | 89.420 | 1.00 | 31.66 |
| ATOM | 9232 | CG | LYS | B | 423 | 88.821 | 68.130 | 90.813 | 1.00 | 34.36 |
| ATOM | 9233 | CD | LYS | B | 423 | 88.615 | 66.591 | 90.727 | 1.00 | 38.25 |
| ATOM | 9234 | CE | LYS | B | 423 | 89.054 | 65.847 | 92.039 | 1.00 | 42.76 |
| ATOM | 9235 | NZ | LYS | B | 423 | 88.749 | 64.333 | 92.051 | 1.00 | 41.23 |
| ATOM | 9236 | N | GLY | B | 424 | 88.557 | 71.946 | 87.662 | 1.00 | 29.74 |
| ATOM | 9237 | CA | GLY | B | 424 | 88.680 | 72.460 | 86.311 | 1.00 | 29.83 |
| ATOM | 9238 | C | GLY | B | 424 | 88.062 | 71.569 | 85.238 | 1.00 | 29.16 |
| ATOM | 9239 | O | GLY | B | 424 | 88.463 | 71.667 | 84.113 | 1.00 | 29.94 |
| ATOM | 9240 | N | MET | B | 425 | 87.122 | 70.697 | 85.586 | 1.00 | 29.00 |
| ATOM | 9241 | CA | MET | B | 425 | 86.456 | 69.773 | 84.652 | 1.00 | 29.38 |
| ATOM | 9242 | C | MET | B | 425 | 85.169 | 70.364 | 84.076 | 1.00 | 27.92 |
| ATOM | 9243 | O | MET | B | 425 | 84.164 | 70.399 | 84.708 | 1.00 | 26.93 |
| ATOM | 9244 | CB | MET | B | 425 | 86.124 | 68.479 | 85.402 | 1.00 | 31.16 |
| ATOM | 9245 | CG | MET | B | 425 | 87.373 | 67.743 | 85.946 | 1.00 | 35.72 |
| ATOM | 9246 | SD | MET | B | 425 | 87.074 | 66.168 | 86.864 | 1.00 | 44.07 |
| ATOM | 9247 | CE | MET | B | 425 | 86.176 | 65.122 | 85.731 | 1.00 | 42.30 |
| ATOM | 9248 | N | PRO | B | 426 | 85.176 | 70.861 | 82.858 | 1.00 | 27.75 |
| ATOM | 9249 | CA | PRO | B | 426 | 83.961 | 71.487 | 82.344 | 1.00 | 26.75 |
| ATOM | 9250 | C | PRO | B | 426 | 82.736 | 70.537 | 82.296 | 1.00 | 26.04 |
| ATOM | 9251 | O | PRO | B | 426 | 81.585 | 70.974 | 82.330 | 1.00 | 25.05 |
| ATOM | 9252 | CB | PRO | B | 426 | 84.395 | 71.964 | 80.963 | 1.00 | 27.69 |
| ATOM | 9253 | CG | PRO | B | 426 | 85.901 | 72.089 | 81.072 | 1.00 | 28.01 |
| ATOM | 9254 | CD | PRO | B | 426 | 86.286 | 70.905 | 81.901 | 1.00 | 27.08 |
| ATOM | 9255 | N | GLY | B | 427 | 82.975 | 69.254 | 82.287 | 1.00 | 24.77 |
| ATOM | 9256 | CA | GLY | B | 427 | 81.901 | 68.298 | 82.172 | 1.00 | 25.53 |
| ATOM | 9257 | C | GLY | B | 427 | 81.412 | 67.795 | 83.514 | 1.00 | 25.53 |
| ATOM | 9258 | O | GLY | B | 427 | 80.577 | 66.894 | 83.557 | 1.00 | 27.27 |
| ATOM | 9259 | N | GLY | B | 428 | 81.911 | 68.353 | 84.600 | 1.00 | 24.46 |
| ATOM | 9260 | CA | GLY | B | 428 | 81.469 | 67.955 | 85.927 | 1.00 | 24.87 |
| ATOM | 9261 | C | GLY | B | 428 | 80.536 | 69.029 | 86.446 | 1.00 | 25.22 |
| ATOM | 9262 | O | GLY | B | 428 | 80.496 | 70.110 | 85.878 | 1.00 | 25.42 |
| ATOM | 9263 | N | ARG | B | 429 | 79.782 | 68.736 | 87.502 | 1.00 | 26.60 |
| ATOM | 9264 | CA | ARG | B | 429 | 78.786 | 69.672 | 88.065 | 1.00 | 26.57 |
| ATOM | 9265 | C | ARG | B | 429 | 78.664 | 69.495 | 89.593 | 1.00 | 26.15 |
| ATOM | 9266 | O | ARG | B | 429 | 78.396 | 68.366 | 90.077 | 1.00 | 25.49 |
| ATOM | 9267 | CB | ARG | B | 429 | 77.398 | 69.441 | 87.461 | 1.00 | 26.67 |
| ATOM | 9268 | CG | ARG | B | 429 | 77.292 | 69.587 | 85.922 | 1.00 | 30.09 |
| ATOM | 9269 | CD | ARG | B | 429 | 76.659 | 70.851 | 85.445 | 1.00 | 29.27 |
| ATOM | 9270 | NE | ARG | B | 429 | 76.286 | 70.790 | 84.037 | 1.00 | 33.96 |
| ATOM | 9271 | CZ | ARG | B | 429 | 75.029 | 70.664 | 83.554 | 1.00 | 37.82 |
| ATOM | 9272 | NH1 | ARG | B | 429 | 73.955 | 70.565 | 84.352 | 1.00 | 36.70 |
| ATOM | 9273 | NH2 | ARG | B | 429 | 74.839 | 70.658 | 82.237 | 1.00 | 40.32 |
| ATOM | 9274 | N | ASN | B | 430 | 78.820 | 70.596 | 90.347 | 1.00 | 24.43 |
| ATOM | 9275 | CA | ASN | B | 430 | 78.633 | 70.524 | 91.796 | 1.00 | 23.95 |
| ATOM | 9276 | C | ASN | B | 430 | 77.848 | 71.705 | 92.315 | 1.00 | 23.68 |
| ATOM | 9277 | O | ASN | B | 430 | 77.730 | 72.743 | 91.648 | 1.00 | 22.09 |
| ATOM | 9278 | CB | ASN | B | 430 | 79.986 | 70.439 | 92.542 | 1.00 | 24.71 |
| ATOM | 9279 | CG | ASN | B | 430 | 80.579 | 69.069 | 92.508 | 1.00 | 21.74 |
| ATOM | 9280 | OD1 | ASN | B | 430 | 80.143 | 68.225 | 93.231 | 1.00 | 23.45 |
| ATOM | 9281 | ND2 | ASN | B | 430 | 81.560 | 68.836 | 91.620 | 1.00 | 23.43 |
| ATOM | 9282 | N | LEU | B | 431 | 77.276 | 71.506 | 93.501 | 1.00 | 23.76 |
| ATOM | 9283 | CA | LEU | B | 431 | 76.518 | 72.517 | 94.194 | 1.00 | 23.69 |
| ATOM | 9284 | C | LEU | B | 431 | 77.396 | 73.352 | 95.107 | 1.00 | 24.86 |
| ATOM | 9285 | O | LEU | B | 431 | 78.162 | 72.784 | 95.904 | 1.00 | 24.09 |
| ATOM | 9286 | CB | LEU | B | 431 | 75.524 | 71.809 | 95.081 | 1.00 | 23.99 |
| ATOM | 9287 | CG | LEU | B | 431 | 74.567 | 72.713 | 95.859 | 1.00 | 21.86 |
| ATOM | 9288 | CD1 | LEU | B | 431 | 73.714 | 73.587 | 94.974 | 1.00 | 22.04 |
| ATOM | 9289 | CD2 | LEU | B | 431 | 73.682 | 71.849 | 96.637 | 1.00 | 21.87 |
| ATOM | 9290 | N | TYR | B | 432 | 77.267 | 74.679 | 95.031 | 1.00 | 25.52 |
| ATOM | 9291 | CA | TYR | B | 432 | 77.987 | 75.589 | 95.942 | 1.00 | 25.38 |
| ATOM | 9292 | C | TYR | B | 432 | 77.034 | 76.564 | 96.670 | 1.00 | 26.29 |
| ATOM | 9293 | O | TYR | B | 432 | 75.879 | 76.822 | 96.267 | 1.00 | 26.62 |
| ATOM | 9294 | CB | TYR | B | 432 | 79.083 | 76.397 | 95.231 | 1.00 | 25.14 |
| ATOM | 9295 | CG | TYR | B | 432 | 80.157 | 75.564 | 94.491 | 1.00 | 26.70 |
| ATOM | 9296 | CD1 | TYR | B | 432 | 79.870 | 74.928 | 93.298 | 1.00 | 25.29 |
| ATOM | 9297 | CD2 | TYR | B | 432 | 81.461 | 75.459 | 94.981 | 1.00 | 29.33 |
| ATOM | 9298 | CE1 | TYR | B | 432 | 80.811 | 74.172 | 92.643 | 1.00 | 26.84 |
| ATOM | 9299 | CE2 | TYR | B | 432 | 82.442 | 74.723 | 94.302 | 1.00 | 28.11 |
| ATOM | 9300 | CZ | TYR | B | 432 | 82.103 | 74.074 | 93.146 | 1.00 | 27.94 |
| ATOM | 9301 | OH | TYR | B | 432 | 83.042 | 73.324 | 92.479 | 1.00 | 27.73 |
| ATOM | 9302 | N | LYS | B | 433 | 77.586 | 77.137 | 97.715 | 1.00 | 26.07 |
| ATOM | 9303 | CA | LYS | B | 433 | 76.914 | 78.002 | 98.601 | 1.00 | 28.02 |
| ATOM | 9304 | C | LYS | B | 433 | 77.777 | 79.265 | 98.873 | 1.00 | 28.15 |
| ATOM | 9305 | O | LYS | B | 433 | 78.951 | 79.179 | 99.267 | 1.00 | 28.25 |
| ATOM | 9306 | CB | LYS | B | 433 | 76.702 | 77.185 | 99.868 | 1.00 | 27.35 |
| ATOM | 9307 | CG | LYS | B | 433 | 75.957 | 77.846 | 100.956 | 1.00 | 31.09 |
| ATOM | 9308 | CD | LYS | B | 433 | 76.123 | 77.047 | 102.240 | 1.00 | 34.56 |
| ATOM | 9309 | CE | LYS | B | 433 | 75.152 | 77.578 | 103.272 | 1.00 | 36.16 |
| ATOM | 9310 | NZ | LYS | B | 433 | 75.234 | 76.877 | 104.562 | 1.00 | 34.35 |
| ATOM | 9311 | N | ILE | B | 434 | 77.195 | 80.431 | 98.653 | 1.00 | 27.94 |
| ATOM | 9312 | CA | ILE | B | 434 | 77.888 | 81.680 | 98.925 | 1.00 | 28.10 |
| ATOM | 9313 | C | ILE | B | 434 | 77.183 | 82.400 | 100.044 | 1.00 | 27.39 |
| ATOM | 9314 | O | ILE | B | 434 | 75.950 | 82.589 | 99.989 | 1.00 | 25.69 |
| ATOM | 9315 | CB | ILE | B | 434 | 77.829 | 82.611 | 97.742 | 1.00 | 28.57 |
| ATOM | 9316 | CG1 | ILE | B | 434 | 78.135 | 81.917 | 96.436 | 1.00 | 28.81 |
| ATOM | 9317 | CG2 | ILE | B | 434 | 78.818 | 83.742 | 97.905 | 1.00 | 29.92 |
| ATOM | 9318 | CD1 | ILE | B | 434 | 77.749 | 82.790 | 95.262 | 1.00 | 29.33 |
| ATOM | 9319 | N | GLN | B | 435 | 77.955 | 82.817 | 101.039 | 1.00 | 27.61 |
| ATOM | 9320 | CA | GLN | B | 435 | 77.449 | 83.693 | 102.094 | 1.00 | 28.32 |
| ATOM | 9321 | C | GLN | B | 435 | 77.384 | 85.069 | 101.533 | 1.00 | 27.86 |
| ATOM | 9322 | O | GLN | B | 435 | 78.388 | 85.589 | 101.142 | 1.00 | 28.87 |
| ATOM | 9323 | CB | GLN | B | 435 | 78.415 | 83.792 | 103.274 | 1.00 | 29.60 |
| ATOM | 9324 | CG | GLN | B | 435 | 78.708 | 82.549 | 103.997 | 1.00 | 32.31 |
| ATOM | 9325 | CD | GLN | B | 435 | 79.361 | 82.786 | 105.354 | 1.00 | 39.38 |
| ATOM | 9326 | OE1 | GLN | B | 435 | 78.832 | 83.531 | 106.192 | 1.00 | 40.29 |
| ATOM | 9327 | NE2 | GLN | B | 435 | 80.488 | 82.107 | 105.592 | 1.00 | 35.81 |
| ATOM | 9328 | N | LEU | B | 436 | 76.224 | 85.688 | 101.526 | 1.00 | 28.49 |
| ATOM | 9329 | CA | LEU | B | 436 | 76.063 | 86.996 | 100.920 | 1.00 | 28.61 |
| ATOM | 9330 | C | LEU | B | 436 | 76.790 | 88.146 | 101.680 | 1.00 | 29.22 |
| ATOM | 9331 | O | LEU | B | 436 | 76.972 | 89.207 | 101.126 | 1.00 | 28.54 |
| ATOM | 9332 | CB | LEU | B | 436 | 74.554 | 87.255 | 100.718 | 1.00 | 28.45 |
| ATOM | 9333 | CG | LEU | B | 436 | 73.897 | 86.104 | 99.912 | 1.00 | 28.99 |
| ATOM | 9334 | CD1 | LEU | B | 436 | 72.411 | 86.225 | 99.772 | 1.00 | 29.93 |
| ATOM | 9335 | CD2 | LEU | B | 436 | 74.557 | 86.016 | 98.503 | 1.00 | 31.60 |
| ATOM | 9336 | N | SER | B | 437 | 77.220 | 87.914 | 102.916 | 1.00 | 29.84 |
| ATOM | 9337 | CA | SER | B | 437 | 77.941 | 88.904 | 103.708 | 1.00 | 31.25 |
| ATOM | 9338 | C | SER | B | 437 | 79.450 | 89.025 | 103.325 | 1.00 | 31.20 |
| ATOM | 9339 | O | SER | B | 437 | 80.142 | 89.931 | 103.785 | 1.00 | 33.20 |
| ATOM | 9340 | CB | SER | B | 437 | 77.738 | 88.593 | 105.209 | 1.00 | 31.10 |
| ATOM | 9341 | OG | SER | B | 437 | 78.718 | 87.662 | 105.727 | 1.00 | 33.45 |
| ATOM | 9342 | N | ASP | B | 438 | 79.925 | 88.093 | 102.480 | 1.00 | 31.28 |
| ATOM | 9343 | CA | ASP | B | 438 | 81.309 | 88.002 | 102.006 | 1.00 | 31.22 |
| ATOM | 9344 | C | ASP | B | 438 | 81.383 | 86.943 | 100.866 | 1.00 | 31.12 |
| ATOM | 9345 | O | ASP | B | 438 | 81.469 | 85.737 | 101.101 | 1.00 | 29.30 |
| ATOM | 9346 | CB | ASP | B | 438 | 82.279 | 87.641 | 103.134 | 1.00 | 31.63 |
| ATOM | 9347 | CG | ASP | B | 438 | 83.732 | 87.507 | 102.655 | 1.00 | 34.50 |
| ATOM | 9348 | OD1 | ASP | B | 438 | 84.011 | 87.584 | 101.430 | 1.00 | 37.97 |
| ATOM | 9349 | OD2 | ASP | B | 438 | 84.675 | 87.335 | 103.457 | 1.00 | 40.92 |
| ATOM | 9350 | N | TYR | B | 439 | 81.388 | 87.432 | 99.638 | 1.00 | 31.76 |
| ATOM | 9351 | CA | TYR | B | 439 | 81.363 | 86.596 | 98.449 | 1.00 | 32.36 |
| ATOM | 9352 | C | TYR | B | 439 | 82.532 | 85.657 | 98.377 | 1.00 | 33.28 |
| ATOM | 9353 | O | TYR | B | 439 | 82.527 | 84.743 | 97.563 | 1.00 | 33.53 |
| ATOM | 9354 | CB | TYR | B | 439 | 81.310 | 87.472 | 97.182 | 1.00 | 32.45 |
| ATOM | 9355 | CG | TYR | B | 439 | 80.147 | 88.444 | 97.151 | 1.00 | 29.88 |
| ATOM | 9356 | CD1 | TYR | B | 439 | 78.929 | 88.090 | 97.671 | 1.00 | 31.80 |
| ATOM | 9357 | CD2 | TYR | B | 439 | 80.273 | 89.703 | 96.593 | 1.00 | 29.37 |
| ATOM | 9358 | CE1 | TYR | B | 439 | 77.842 | 88.963 | 97.670 | 1.00 | 31.92 |
| ATOM | 9359 | CE2 | TYR | B | 439 | 79.192 | 90.610 | 96.599 | 1.00 | 30.27 |
| ATOM | 9360 | CZ | TYR | B | 439 | 77.980 | 90.205 | 97.126 | 1.00 | 32.14 |
| ATOM | 9361 | OH | TYR | B | 439 | 76.891 | 91.018 | 97.115 | 1.00 | 32.51 |
| ATOM | 9362 | N | THR | B | 440 | 83.535 | 85.827 | 99.237 | 1.00 | 33.58 |
| ATOM | 9363 | CA | THR | B | 440 | 84.728 | 84.991 | 99.124 | 1.00 | 33.41 |
| ATOM | 9364 | C | THR | B | 440 | 84.504 | 83.761 | 99.940 | 1.00 | 33.90 |
| ATOM | 9365 | O | THR | B | 440 | 85.247 | 82.801 | 99.862 | 1.00 | 33.02 |
| ATOM | 9366 | CB | THR | B | 440 | 85.969 | 85.708 | 99.642 | 1.00 | 34.51 |
| ATOM | 9367 | OG1 | THR | B | 440 | 85.790 | 86.038 | 101.022 | 1.00 | 33.37 |
| ATOM | 9368 | CG2 | THR | B | 440 | 86.162 | 87.058 | 98.952 | 1.00 | 35.23 |
| ATOM | 9369 | N | LYS | B | 441 | 83.476 | 83.797 | 100.760 | 1.00 | 34.65 |
| ATOM | 9370 | CA | LYS | B | 441 | 83.162 | 82.649 | 101.570 | 1.00 | 35.47 |
| ATOM | 9371 | C | LYS | B | 441 | 82.248 | 81.768 | 100.779 | 1.00 | 34.84 |
| ATOM | 9372 | O | LYS | B | 441 | 81.042 | 81.992 | 100.765 | 1.00 | 34.38 |
| ATOM | 9373 | CB | LYS | B | 441 | 82.488 | 83.077 | 102.868 | 1.00 | 36.77 |
| ATOM | 9374 | CG | LYS | B | 441 | 83.412 | 83.807 | 103.801 | 1.00 | 40.34 |
| ATOM | 9375 | CD | LYS | B | 441 | 82.825 | 83.897 | 105.197 | 1.00 | 46.20 |
| ATOM | 9376 | CE | LYS | B | 441 | 83.650 | 84.855 | 106.066 | 1.00 | 49.13 |
| ATOM | 9377 | NZ | LYS | B | 441 | 82.871 | 85.350 | 107.231 | 1.00 | 52.95 |
| ATOM | 9378 | N | VAL | B | 442 | 82.836 | 80.749 | 100.154 | 1.00 | 34.86 |
| ATOM | 9379 | CA | VAL | B | 442 | 82.139 | 79.842 | 99.268 | 1.00 | 34.00 |
| ATOM | 9380 | C | VAL | B | 442 | 82.481 | 78.404 | 99.590 | 1.00 | 34.08 |
| ATOM | 9381 | O | VAL | B | 442 | 83.635 | 78.005 | 99.555 | 1.00 | 33.95 |
| ATOM | 9382 | CB | VAL | B | 442 | 82.496 | 80.051 | 97.796 | 1.00 | 34.34 |
| ATOM | 9383 | CG1 | VAL | B | 442 | 81.530 | 79.260 | 96.932 | 1.00 | 34.20 |
| ATOM | 9384 | CG2 | VAL | B | 442 | 82.433 | 81.507 | 97.403 | 1.00 | 33.73 |
| ATOM | 9385 | N | THR | B | 443 | 81.449 | 77.624 | 99.863 | 1.00 | 33.29 |
| ATOM | 9386 | CA | THR | B | 443 | 81.594 | 76.226 | 100.159 | 1.00 | 33.89 |
| ATOM | 9387 | C | THR | B | 443 | 81.036 | 75.347 | 99.037 | 1.00 | 33.32 |
| ATOM | 9388 | O | THR | B | 443 | 79.954 | 75.600 | 98.506 | 1.00 | 32.30 |
| ATOM | 9389 | CB | THR | B | 443 | 80.803 | 75.883 | 101.428 | 1.00 | 33.93 |
| ATOM | 9390 | OG1 | THR | B | 443 | 81.518 | 76.324 | 102.588 | 1.00 | 36.06 |
| ATOM | 9391 | CG2 | THR | B | 443 | 80.764 | 74.405 | 101.620 | 1.00 | 36.80 |
| ATOM | 9392 | N | CYS | B | 444 | 81.769 | 74.303 | 98.702 | 1.00 | 32.49 |
| ATOM | 9393 | CA | CYS | B | 444 | 81.255 | 73.315 | 97.792 | 1.00 | 32.00 |
| ATOM | 9394 | C | CYS | B | 444 | 80.508 | 72.321 | 98.639 | 1.00 | 30.90 |
| ATOM | 9395 | O | CYS | B | 444 | 81.100 | 71.700 | 99.497 | 1.00 | 31.13 |
| ATOM | 9396 | CB | CYS | B | 444 | 82.345 | 72.599 | 97.034 | 1.00 | 31.25 |
| ATOM | 9397 | SG | CYS | B | 444 | 81.517 | 71.608 | 95.740 | 1.00 | 33.54 |
| ATOM | 9398 | N | LEU | B | 445 | 79.228 | 72.138 | 98.395 | 1.00 | 29.70 |
| ATOM | 9399 | CA | LEU | B | 445 | 78.449 | 71.269 | 99.248 | 1.00 | 29.76 |
| ATOM | 9400 | C | LEU | B | 445 | 78.429 | 69.824 | 98.811 | 1.00 | 29.48 |
| ATOM | 9401 | O | LEU | B | 445 | 78.045 | 68.955 | 99.607 | 1.00 | 29.20 |
| ATOM | 9402 | CB | LEU | B | 445 | 76.985 | 71.752 | 99.351 | 1.00 | 29.21 |
| ATOM | 9403 | CG | LEU | B | 445 | 76.725 | 73.159 | 99.886 | 1.00 | 30.36 |
| ATOM | 9404 | CD1 | LEU | B | 445 | 75.235 | 73.460 | 100.030 | 1.00 | 29.62 |
| ATOM | 9405 | CD2 | LEU | B | 445 | 77.393 | 73.317 | 101.258 | 1.00 | 32.84 |
| ATOM | 9406 | N | SER | B | 446 | 78.819 | 69.546 | 97.574 | 1.00 | 28.92 |
| ATOM | 9407 | CA | SER | B | 446 | 78.678 | 68.204 | 97.053 | 1.00 | 29.28 |
| ATOM | 9408 | C | SER | B | 446 | 79.976 | 67.570 | 96.576 | 1.00 | 30.53 |
| ATOM | 9409 | O | SER | B | 446 | 80.015 | 66.361 | 96.435 | 1.00 | 30.85 |
| ATOM | 9410 | CB | SER | B | 446 | 77.697 | 68.216 | 95.866 | 1.00 | 29.34 |
| ATOM | 9411 | OG | SER | B | 446 | 78.343 | 68.793 | 94.730 | 1.00 | 25.10 |
| ATOM | 9412 | N | CYS | B | 447 | 81.006 | 68.377 | 96.313 | 1.00 | 31.43 |
| ATOM | 9413 | CA | CYS | B | 447 | 82.256 | 67.903 | 95.699 | 1.00 | 33.66 |
| ATOM | 9414 | C | CYS | B | 447 | 82.850 | 66.676 | 96.379 | 1.00 | 33.76 |
| ATOM | 9415 | O | CYS | B | 447 | 83.270 | 65.747 | 95.723 | 1.00 | 34.81 |
| ATOM | 9416 | CB | CYS | B | 447 | 83.336 | 69.024 | 95.735 | 1.00 | 34.48 |
| ATOM | 9417 | SG | CYS | B | 447 | 82.901 | 70.477 | 94.785 | 1.00 | 37.87 |
| ATOM | 9418 | N | GLU | B | 448 | 82.878 | 66.673 | 97.694 | 1.00 | 33.01 |
| ATOM | 9419 | CA | GLU | B | 448 | 83.555 | 65.608 | 98.408 | 1.00 | 34.22 |
| ATOM | 9420 | C | GLU | B | 448 | 82.643 | 64.621 | 99.083 | 1.00 | 33.60 |
| ATOM | 9421 | O | GLU | B | 448 | 83.107 | 63.862 | 99.927 | 1.00 | 33.36 |
| ATOM | 9422 | CB | GLU | B | 448 | 84.444 | 66.194 | 99.498 | 1.00 | 34.39 |
| ATOM | 9423 | CG | GLU | B | 448 | 85.418 | 67.212 | 98.982 | 1.00 | 38.17 |
| ATOM | 9424 | CD | GLU | B | 448 | 86.415 | 66.626 | 98.017 | 1.00 | 40.37 |
| ATOM | 9425 | OE1 | GLU | B | 448 | 86.776 | 65.442 | 98.173 | 1.00 | 45.35 |
| ATOM | 9426 | OE2 | GLU | B | 448 | 86.858 | 67.372 | 97.118 | 1.00 | 44.29 |
| ATOM | 9427 | N | LEU | B | 449 | 81.370 | 64.598 | 98.715 | 1.00 | 33.47 |
| ATOM | 9428 | CA | LEU | B | 449 | 80.439 | 63.717 | 99.420 | 1.00 | 33.91 |
| ATOM | 9429 | C | LEU | B | 449 | 80.726 | 62.247 | 99.103 | 1.00 | 34.37 |
| ATOM | 9430 | O | LEU | B | 449 | 80.551 | 61.374 | 99.955 | 1.00 | 33.40 |
| ATOM | 9431 | CB | LEU | B | 449 | 78.979 | 64.029 | 99.032 | 1.00 | 33.56 |
| ATOM | 9432 | CG | LEU | B | 449 | 78.270 | 65.247 | 99.619 | 1.00 | 34.92 |
| ATOM | 9433 | CD1 | LEU | B | 449 | 76.909 | 65.461 | 98.934 | 1.00 | 37.34 |
| ATOM | 9434 | CD2 | LEU | B | 449 | 78.067 | 65.109 | 101.125 | 1.00 | 34.58 |
| ATOM | 9435 | N | ASN | B | 450 | 81.113 | 61.996 | 97.852 | 1.00 | 34.40 |
| ATOM | 9436 | CA | ASN | B | 450 | 81.286 | 60.653 | 97.331 | 1.00 | 35.35 |
| ATOM | 9437 | C | ASN | B | 450 | 82.036 | 60.852 | 96.017 | 1.00 | 34.66 |
| ATOM | 9438 | O | ASN | B | 450 | 81.498 | 60.567 | 94.969 | 1.00 | 34.27 |
| ATOM | 9439 | CB | ASN | B | 450 | 79.911 | 60.067 | 96.990 | 1.00 | 35.97 |
| ATOM | 9440 | CG | ASN | B | 450 | 79.281 | 59.263 | 98.097 | 1.00 | 40.57 |
| ATOM | 9441 | OD1 | ASN | B | 450 | 79.803 | 58.213 | 98.492 | 1.00 | 44.98 |
| ATOM | 9442 | ND2 | ASN | B | 450 | 78.079 | 59.697 | 98.546 | 1.00 | 42.91 |
| ATOM | 9443 | N | PRO | B | 451 | 83.285 | 61.294 | 96.085 | 1.00 | 35.39 |
| ATOM | 9444 | CA | PRO | B | 451 | 84.036 | 61.820 | 94.919 | 1.00 | 34.83 |
| ATOM | 9445 | C | PRO | B | 451 | 84.399 | 60.928 | 93.726 | 1.00 | 35.41 |
| ATOM | 9446 | O | PRO | B | 451 | 84.689 | 61.437 | 92.662 | 1.00 | 34.70 |
| ATOM | 9447 | CB | PRO | B | 451 | 85.347 | 62.340 | 95.529 | 1.00 | 34.92 |
| ATOM | 9448 | CG | PRO | B | 451 | 85.310 | 62.005 | 97.016 | 1.00 | 36.21 |
| ATOM | 9449 | CD | PRO | B | 451 | 84.081 | 61.259 | 97.337 | 1.00 | 35.15 |
| ATOM | 9450 | N | GLU | B | 452 | 84.468 | 59.625 | 93.899 | 1.00 | 36.27 |
| ATOM | 9451 | CA | GLU | B | 452 | 84.771 | 58.782 | 92.769 | 1.00 | 36.68 |
| ATOM | 9452 | C | GLU | B | 452 | 83.504 | 58.377 | 92.047 | 1.00 | 35.32 |
| ATOM | 9453 | O | GLU | B | 452 | 83.536 | 58.042 | 90.871 | 1.00 | 35.61 |
| ATOM | 9454 | CB | GLU | B | 452 | 85.459 | 57.517 | 93.226 | 1.00 | 37.91 |
| ATOM | 9455 | CG | GLU | B | 452 | 86.958 | 57.644 | 93.324 | 1.00 | 41.25 |
| ATOM | 9456 | CD | GLU | B | 452 | 87.396 | 57.480 | 94.747 | 1.00 | 46.23 |
| ATOM | 9457 | OE1 | GLU | B | 452 | 87.352 | 56.315 | 95.221 | 1.00 | 50.42 |
| ATOM | 9458 | OE2 | GLU | B | 452 | 87.757 | 58.506 | 95.379 | 1.00 | 48.73 |
| ATOM | 9459 | N | ARG | B | 453 | 82.399 | 58.368 | 92.780 | 1.00 | 33.86 |
| ATOM | 9460 | CA | ARG | B | 453 | 81.134 | 57.969 | 92.228 | 1.00 | 32.45 |
| ATOM | 9461 | C | ARG | B | 453 | 80.325 | 59.160 | 91.742 | 1.00 | 31.92 |
| ATOM | 9462 | O | ARG | B | 453 | 79.527 | 59.049 | 90.813 | 1.00 | 30.67 |
| ATOM | 9463 | CB | ARG | B | 453 | 80.330 | 57.222 | 93.275 | 1.00 | 32.79 |
| ATOM | 9464 | CG | ARG | B | 453 | 79.002 | 56.646 | 92.768 | 1.00 | 31.10 |
| ATOM | 9465 | CD | ARG | B | 453 | 78.183 | 56.114 | 93.884 | 1.00 | 32.75 |
| ATOM | 9466 | NE | ARG | B | 453 | 76.827 | 55.806 | 93.497 | 1.00 | 34.45 |
| ATOM | 9467 | CZ | ARG | B | 453 | 76.050 | 54.974 | 94.169 | 1.00 | 33.89 |
| ATOM | 9468 | NH1 | ARG | B | 453 | 76.521 | 54.366 | 95.252 | 1.00 | 31.33 |
| ATOM | 9469 | NH2 | ARG | B | 453 | 74.803 | 54.737 | 93.766 | 1.00 | 31.42 |
| ATOM | 9470 | N | CYS | B | 454 | 80.537 | 60.311 | 92.348 | 1.00 | 30.98 |
| ATOM | 9471 | CA | CYS | B | 454 | 79.628 | 61.416 | 92.077 | 1.00 | 30.23 |
| ATOM | 9472 | C | CYS | B | 454 | 80.330 | 62.698 | 91.739 | 1.00 | 28.75 |
| ATOM | 9473 | O | CYS | B | 454 | 80.968 | 63.299 | 92.581 | 1.00 | 28.32 |
| ATOM | 9474 | CB | CYS | B | 454 | 78.722 | 61.596 | 93.271 | 1.00 | 30.58 |
| ATOM | 9475 | SG | CYS | B | 454 | 77.582 | 60.243 | 93.475 | 1.00 | 30.80 |
| ATOM | 9476 | N | GLN | B | 455 | 80.198 | 63.100 | 90.479 | 1.00 | 27.50 |
| ATOM | 9477 | CA | GLN | B | 455 | 80.859 | 64.284 | 89.977 | 1.00 | 26.27 |
| ATOM | 9478 | C | GLN | B | 455 | 79.901 | 65.140 | 89.143 | 1.00 | 25.48 |
| ATOM | 9479 | O | GLN | B | 455 | 80.327 | 66.147 | 88.624 | 1.00 | 25.25 |
| ATOM | 9480 | CB | GLN | B | 455 | 82.092 | 63.894 | 89.132 | 1.00 | 25.42 |
| ATOM | 9481 | CG | GLN | B | 455 | 83.283 | 63.322 | 89.864 | 1.00 | 27.34 |
| ATOM | 9482 | CD | GLN | B | 455 | 84.294 | 62.520 | 88.937 | 1.00 | 32.33 |
| ATOM | 9483 | OE1 | GLN | B | 455 | 84.123 | 62.440 | 87.732 | 1.00 | 32.86 |
| ATOM | 9484 | NE2 | GLN | B | 455 | 85.320 | 61.948 | 89.535 | 1.00 | 31.75 |
| ATOM | 9485 | N | TYR | B | 456 | 78.637 | 64.728 | 89.008 | 1.00 | 25.74 |
| ATOM | 9486 | CA | TYR | B | 456 | 77.616 | 65.451 | 88.214 | 1.00 | 26.19 |
| ATOM | 9487 | C | TYR | B | 456 | 76.305 | 65.566 | 89.017 | 1.00 | 26.13 |
| ATOM | 9488 | O | TYR | B | 456 | 75.471 | 64.642 | 89.062 | 1.00 | 26.14 |
| ATOM | 9489 | CB | TYR | B | 456 | 77.336 | 64.745 | 86.880 | 1.00 | 26.83 |
| ATOM | 9490 | CG | TYR | B | 456 | 76.775 | 65.604 | 85.763 | 1.00 | 24.10 |
| ATOM | 9491 | CD1 | TYR | B | 456 | 75.408 | 65.856 | 85.647 | 1.00 | 25.09 |
| ATOM | 9492 | CD2 | TYR | B | 456 | 77.613 | 66.155 | 84.811 | 1.00 | 25.81 |
| ATOM | 9493 | CE1 | TYR | B | 456 | 74.884 | 66.645 | 84.564 | 1.00 | 23.20 |
| ATOM | 9494 | CE2 | TYR | B | 456 | 77.120 | 66.931 | 83.752 | 1.00 | 22.75 |
| ATOM | 9495 | CZ | TYR | B | 456 | 75.759 | 67.171 | 83.632 | 1.00 | 24.21 |
| ATOM | 9496 | OH | TYR | B | 456 | 75.326 | 67.971 | 82.582 | 1.00 | 23.14 |
| ATOM | 9497 | N | TYR | B | 457 | 76.126 | 66.699 | 89.668 | 1.00 | 25.26 |
| ATOM | 9498 | CA | TYR | B | 457 | 74.969 | 66.874 | 90.535 | 1.00 | 24.93 |
| ATOM | 9499 | C | TYR | B | 457 | 73.966 | 67.883 | 89.981 | 1.00 | 25.41 |
| ATOM | 9500 | O | TYR | B | 457 | 74.353 | 68.823 | 89.273 | 1.00 | 24.40 |
| ATOM | 9501 | CB | TYR | B | 457 | 75.416 | 67.413 | 91.887 | 1.00 | 25.11 |
| ATOM | 9502 | CG | TYR | B | 457 | 76.131 | 66.426 | 92.804 | 1.00 | 24.76 |
| ATOM | 9503 | CD1 | TYR | B | 457 | 75.407 | 65.643 | 93.682 | 1.00 | 26.61 |
| ATOM | 9504 | CD2 | TYR | B | 457 | 77.505 | 66.326 | 92.832 | 1.00 | 23.91 |
| ATOM | 9505 | CE1 | TYR | B | 457 | 76.013 | 64.761 | 94.544 | 1.00 | 25.28 |
| ATOM | 9506 | CE2 | TYR | B | 457 | 78.142 | 65.414 | 93.694 | 1.00 | 26.22 |
| ATOM | 9507 | CZ | TYR | B | 457 | 77.378 | 64.647 | 94.538 | 1.00 | 25.74 |
| ATOM | 9508 | OH | TYR | B | 457 | 77.941 | 63.748 | 95.387 | 1.00 | 29.90 |
| ATOM | 9509 | N | SER | B | 458 | 72.684 | 67.629 | 90.267 | 1.00 | 25.31 |
| ATOM | 9510 | CA | SER | B | 458 | 71.622 | 68.618 | 90.197 | 1.00 | 26.57 |
| ATOM | 9511 | C | SER | B | 458 | 70.940 | 68.599 | 91.598 | 1.00 | 26.57 |
| ATOM | 9512 | O | SER | B | 458 | 71.214 | 67.736 | 92.401 | 1.00 | 25.56 |
| ATOM | 9513 | CB | SER | B | 458 | 70.614 | 68.261 | 89.113 | 1.00 | 26.59 |
| ATOM | 9514 | OG | SER | B | 458 | 69.964 | 67.072 | 89.530 | 1.00 | 30.73 |
| ATOM | 9515 | N | VAL | B | 459 | 70.047 | 69.541 | 91.890 | 1.00 | 27.82 |
| ATOM | 9516 | CA | VAL | B | 459 | 69.487 | 69.668 | 93.224 | 1.00 | 27.60 |
| ATOM | 9517 | C | VAL | B | 459 | 68.062 | 70.172 | 93.170 | 1.00 | 27.58 |
| ATOM | 9518 | O | VAL | B | 459 | 67.659 | 70.785 | 92.199 | 1.00 | 25.23 |
| ATOM | 9519 | CB | VAL | B | 459 | 70.333 | 70.681 | 94.044 | 1.00 | 28.32 |
| ATOM | 9520 | CG1 | VAL | B | 459 | 70.239 | 72.074 | 93.432 | 1.00 | 28.21 |
| ATOM | 9521 | CG2 | VAL | B | 459 | 69.956 | 70.722 | 95.464 | 1.00 | 28.98 |
| ATOM | 9522 | N | SER | B | 460 | 67.335 | 69.956 | 94.266 | 1.00 | 27.43 |
| ATOM | 9523 | CA | SER | B | 460 | 65.971 | 70.433 | 94.415 | 1.00 | 28.37 |
| ATOM | 9524 | C | SER | B | 460 | 65.756 | 70.855 | 95.861 | 1.00 | 28.75 |
| ATOM | 9525 | O | SER | B | 460 | 65.780 | 70.030 | 96.748 | 1.00 | 29.69 |
| ATOM | 9526 | CB | SER | B | 460 | 65.001 | 69.318 | 93.967 | 1.00 | 29.10 |
| ATOM | 9527 | OG | SER | B | 460 | 63.716 | 69.512 | 94.466 | 1.00 | 29.79 |
| ATOM | 9528 | N | PHE | B | 461 | 65.597 | 72.150 | 96.096 | 1.00 | 28.74 |
| ATOM | 9529 | CA | PHE | B | 461 | 65.476 | 72.713 | 97.416 | 1.00 | 28.88 |
| ATOM | 9530 | C | PHE | B | 461 | 64.022 | 72.821 | 97.845 | 1.00 | 29.45 |
| ATOM | 9531 | O | PHE | B | 461 | 63.196 | 73.183 | 97.053 | 1.00 | 28.41 |
| ATOM | 9532 | CB | PHE | B | 461 | 66.075 | 74.133 | 97.446 | 1.00 | 28.68 |
| ATOM | 9533 | CG | PHE | B | 461 | 67.584 | 74.176 | 97.514 | 1.00 | 28.23 |
| ATOM | 9534 | CD1 | PHE | B | 461 | 68.236 | 74.124 | 98.718 | 1.00 | 26.64 |
| ATOM | 9535 | CD2 | PHE | B | 461 | 68.342 | 74.261 | 96.371 | 1.00 | 28.54 |
| ATOM | 9536 | CE1 | PHE | B | 461 | 69.598 | 74.160 | 98.786 | 1.00 | 27.72 |
| ATOM | 9537 | CE2 | PHE | B | 461 | 69.735 | 74.294 | 96.440 | 1.00 | 26.16 |
| ATOM | 9538 | CZ | PHE | B | 461 | 70.348 | 74.239 | 97.627 | 1.00 | 26.94 |
| ATOM | 9539 | N | SER | B | 462 | 63.725 | 72.550 | 99.117 | 1.00 | 29.82 |
| ATOM | 9540 | CA | SER | B | 462 | 62.365 | 72.769 | 99.641 | 1.00 | 31.14 |
| ATOM | 9541 | C | SER | B | 462 | 61.999 | 74.273 | 99.620 | 1.00 | 32.09 |
| ATOM | 9542 | O | SER | B | 462 | 62.755 | 75.097 | 99.142 | 1.00 | 30.13 |
| ATOM | 9543 | CB | SER | B | 462 | 62.281 | 72.302 | 101.084 | 1.00 | 29.40 |
| ATOM | 9544 | OG | SER | B | 462 | 63.140 | 73.116 | 101.864 | 1.00 | 29.33 |
| ATOM | 9545 | N | LYS | B | 463 | 60.854 | 74.641 | 100.183 | 1.00 | 34.66 |
| ATOM | 9546 | CA | LYS | B | 463 | 60.551 | 76.073 | 100.309 | 1.00 | 36.75 |
| ATOM | 9547 | C | LYS | B | 463 | 61.505 | 76.702 | 101.311 | 1.00 | 36.72 |
| ATOM | 9548 | O | LYS | B | 463 | 61.863 | 76.081 | 102.329 | 1.00 | 37.78 |
| ATOM | 9549 | CB | LYS | B | 463 | 59.107 | 76.336 | 100.715 | 1.00 | 37.72 |
| ATOM | 9550 | CG | LYS | B | 463 | 58.093 | 76.100 | 99.634 | 1.00 | 40.79 |
| ATOM | 9551 | CD | LYS | B | 463 | 56.699 | 76.615 | 100.104 | 1.00 | 46.74 |
| ATOM | 9552 | CE | LYS | B | 463 | 55.580 | 76.397 | 99.045 | 1.00 | 48.05 |
| ATOM | 9553 | NZ | LYS | B | 463 | 54.302 | 77.053 | 99.434 | 1.00 | 48.81 |
| ATOM | 9554 | N | GLU | B | 464 | 61.952 | 77.917 | 101.008 | 1.00 | 36.96 |
| ATOM | 9555 | CA | GLU | B | 464 | 62.874 | 78.661 | 101.882 | 1.00 | 37.22 |
| ATOM | 9556 | C | GLU | B | 464 | 64.215 | 77.977 | 102.016 | 1.00 | 35.82 |
| ATOM | 9557 | O | GLU | B | 464 | 65.014 | 78.325 | 102.887 | 1.00 | 34.26 |
| ATOM | 9558 | CB | GLU | B | 464 | 62.326 | 78.889 | 103.291 | 1.00 | 38.30 |
| ATOM | 9559 | CG | GLU | B | 464 | 60.963 | 79.522 | 103.408 | 1.00 | 43.61 |
| ATOM | 9560 | CD | GLU | B | 464 | 60.676 | 79.872 | 104.853 | 1.00 | 50.71 |
| ATOM | 9561 | OE1 | GLU | B | 464 | 60.442 | 78.922 | 105.651 | 1.00 | 55.30 |
| ATOM | 9562 | OE2 | GLU | B | 464 | 60.724 | 81.087 | 105.204 | 1.00 | 53.88 |
| ATOM | 9563 | N | ALA | B | 465 | 64.465 | 77.006 | 101.148 | 1.00 | 35.12 |
| ATOM | 9564 | CA | ALA | B | 465 | 65.737 | 76.298 | 101.157 | 1.00 | 34.85 |
| ATOM | 9565 | C | ALA | B | 465 | 66.163 | 75.705 | 102.523 | 1.00 | 34.02 |
| ATOM | 9566 | O | ALA | B | 465 | 67.347 | 75.684 | 102.857 | 1.00 | 33.87 |
| ATOM | 9567 | CB | ALA | B | 465 | 66.832 | 77.210 | 100.607 | 1.00 | 35.11 |
| ATOM | 9568 | N | LYS | B | 466 | 65.204 | 75.158 | 103.268 | 1.00 | 34.17 |
| ATOM | 9569 | CA | LYS | B | 466 | 65.492 | 74.466 | 104.537 | 1.00 | 34.05 |
| ATOM | 9570 | C | LYS | B | 466 | 66.128 | 73.096 | 104.314 | 1.00 | 33.03 |
| ATOM | 9571 | O | LYS | B | 466 | 66.928 | 72.628 | 105.126 | 1.00 | 33.44 |
| ATOM | 9572 | CB | LYS | B | 466 | 64.214 | 74.266 | 105.322 | 1.00 | 34.59 |
| ATOM | 9573 | CG | LYS | B | 466 | 64.380 | 74.568 | 106.755 | 1.00 | 37.18 |
| ATOM | 9574 | CD | LYS | B | 466 | 63.201 | 74.139 | 107.580 | 1.00 | 41.58 |
| ATOM | 9575 | CE | LYS | B | 466 | 63.616 | 73.940 | 109.037 | 1.00 | 43.50 |
| ATOM | 9576 | NZ | LYS | B | 466 | 62.774 | 72.833 | 109.527 | 1.00 | 45.13 |
| ATOM | 9577 | N | TYR | B | 467 | 65.747 | 72.462 | 103.215 | 1.00 | 31.59 |
| ATOM | 9578 | CA | TYR | B | 467 | 66.300 | 71.177 | 102.860 | 1.00 | 30.73 |
| ATOM | 9579 | C | TYR | B | 467 | 66.474 | 71.070 | 101.358 | 1.00 | 29.96 |
| ATOM | 9580 | O | TYR | B | 467 | 65.889 | 71.837 | 100.604 | 1.00 | 28.87 |
| ATOM | 9581 | CB | TYR | B | 467 | 65.382 | 70.042 | 103.319 | 1.00 | 31.25 |
| ATOM | 9582 | CG | TYR | B | 467 | 65.066 | 70.028 | 104.796 | 1.00 | 30.99 |
| ATOM | 9583 | CD1 | TYR | B | 467 | 63.998 | 70.738 | 105.304 | 1.00 | 33.27 |
| ATOM | 9584 | CD2 | TYR | B | 467 | 65.800 | 69.281 | 105.673 | 1.00 | 30.15 |
| ATOM | 9585 | CE1 | TYR | B | 467 | 63.686 | 70.704 | 106.688 | 1.00 | 33.81 |
| ATOM | 9586 | CE2 | TYR | B | 467 | 65.500 | 69.262 | 107.043 | 1.00 | 32.44 |
| ATOM | 9587 | CZ | TYR | B | 467 | 64.443 | 69.986 | 107.533 | 1.00 | 32.21 |
| ATOM | 9588 | OH | TYR | B | 467 | 64.127 | 69.954 | 108.883 | 1.00 | 32.73 |
| ATOM | 9589 | N | TYR | B | 468 | 67.287 | 70.106 | 100.940 | 1.00 | 29.46 |
| ATOM | 9590 | CA | TYR | B | 468 | 67.547 | 69.847 | 99.537 | 1.00 | 28.49 |
| ATOM | 9591 | C | TYR | B | 468 | 67.794 | 68.381 | 99.253 | 1.00 | 28.61 |
| ATOM | 9592 | O | TYR | B | 468 | 68.358 | 67.645 | 100.095 | 1.00 | 28.77 |
| ATOM | 9593 | CB | TYR | B | 468 | 68.707 | 70.709 | 99.012 | 1.00 | 27.78 |
| ATOM | 9594 | CG | TYR | B | 468 | 70.088 | 70.640 | 99.691 | 1.00 | 28.05 |
| ATOM | 9595 | CD1 | TYR | B | 468 | 70.398 | 71.471 | 100.759 | 1.00 | 28.76 |
| ATOM | 9596 | CD2 | TYR | B | 468 | 71.101 | 69.816 | 99.211 | 1.00 | 28.66 |
| ATOM | 9597 | CE1 | TYR | B | 468 | 71.641 | 71.444 | 101.357 | 1.00 | 28.54 |
| ATOM | 9598 | CE2 | TYR | B | 468 | 72.367 | 69.793 | 99.805 | 1.00 | 28.13 |
| ATOM | 9599 | CZ | TYR | B | 468 | 72.617 | 70.623 | 100.880 | 1.00 | 27.70 |
| ATOM | 9600 | OH | TYR | B | 468 | 73.837 | 70.630 | 101.527 | 1.00 | 27.01 |
| ATOM | 9601 | N | GLN | B | 469 | 67.333 | 67.947 | 98.078 | 1.00 | 28.39 |
| ATOM | 9602 | CA | GLN | B | 469 | 67.655 | 66.620 | 97.552 | 1.00 | 27.88 |
| ATOM | 9603 | C | GLN | B | 469 | 68.787 | 66.762 | 96.570 | 1.00 | 27.66 |
| ATOM | 9604 | O | GLN | B | 469 | 68.701 | 67.579 | 95.670 | 1.00 | 26.59 |
| ATOM | 9605 | CB | GLN | B | 469 | 66.480 | 66.001 | 96.776 | 1.00 | 28.62 |
| ATOM | 9606 | CG | GLN | B | 469 | 66.748 | 64.572 | 96.227 | 1.00 | 26.39 |
| ATOM | 9607 | CD | GLN | B | 469 | 65.749 | 64.143 | 95.130 | 1.00 | 28.98 |
| ATOM | 9608 | OE1 | GLN | B | 469 | 65.374 | 64.939 | 94.301 | 1.00 | 31.48 |
| ATOM | 9609 | NE2 | GLN | B | 469 | 65.268 | 62.896 | 95.193 | 1.00 | 29.61 |
| ATOM | 9610 | N | LEU | B | 470 | 69.840 | 65.968 | 96.730 | 1.00 | 27.61 |
| ATOM | 9611 | CA | LEU | B | 470 | 70.898 | 65.928 | 95.754 | 1.00 | 28.28 |
| ATOM | 9612 | C | LEU | B | 470 | 70.746 | 64.731 | 94.849 | 1.00 | 28.23 |
| ATOM | 9613 | O | LEU | B | 470 | 70.341 | 63.656 | 95.304 | 1.00 | 27.32 |
| ATOM | 9614 | CB | LEU | B | 470 | 72.279 | 65.892 | 96.388 | 1.00 | 27.67 |
| ATOM | 9615 | CG | LEU | B | 470 | 72.785 | 67.229 | 96.904 | 1.00 | 30.41 |
| ATOM | 9616 | CD1 | LEU | B | 470 | 74.044 | 66.988 | 97.626 | 1.00 | 28.59 |
| ATOM | 9617 | CD2 | LEU | B | 470 | 72.985 | 68.289 | 95.801 | 1.00 | 31.31 |
| ATOM | 9618 | N | ARG | B | 471 | 71.059 | 64.950 | 93.568 | 1.00 | 28.24 |
| ATOM | 9619 | CA | ARG | B | 471 | 70.967 | 63.945 | 92.522 | 1.00 | 29.03 |
| ATOM | 9620 | C | ARG | B | 471 | 72.295 | 63.882 | 91.765 | 1.00 | 29.35 |
| ATOM | 9621 | O | ARG | B | 471 | 72.635 | 64.753 | 90.965 | 1.00 | 26.50 |
| ATOM | 9622 | CB | ARG | B | 471 | 69.859 | 64.231 | 91.485 | 1.00 | 30.47 |
| ATOM | 9623 | CG | ARG | B | 471 | 70.105 | 63.401 | 90.148 | 1.00 | 35.53 |
| ATOM | 9624 | CD | ARG | B | 471 | 68.872 | 63.172 | 89.177 | 1.00 | 42.75 |
| ATOM | 9625 | NE | ARG | B | 471 | 68.849 | 64.130 | 88.078 | 1.00 | 47.16 |
| ATOM | 9626 | CZ | ARG | B | 471 | 67.781 | 64.435 | 87.344 | 1.00 | 51.84 |
| ATOM | 9627 | NE1 | ARG | B | 471 | 66.603 | 63.840 | 87.555 | 1.00 | 52.12 |
| ATOM | 9628 | NH2 | ARG | B | 471 | 67.905 | 65.353 | 86.389 | 1.00 | 51.86 |
| ATOM | 9629 | N | CYS | B | 472 | 73.039 | 62.843 | 92.071 | 1.00 | 29.45 |
| ATOM | 9630 | CA | CYS | B | 472 | 74.273 | 62.560 | 91.411 | 1.00 | 30.96 |
| ATOM | 9631 | C | CYS | B | 472 | 73.955 | 61.712 | 90.186 | 1.00 | 29.94 |
| ATOM | 9632 | O | CYS | B | 472 | 73.263 | 60.743 | 90.315 | 1.00 | 29.73 |
| ATOM | 9633 | CB | CYS | B | 472 | 75.137 | 61.782 | 92.412 | 1.00 | 31.75 |
| ATOM | 9634 | SG | CYS | B | 472 | 76.340 | 60.578 | 91.768 | 1.00 | 36.58 |
| ATOM | 9635 | N | SER | B | 473 | 74.462 | 62.081 | 89.014 | 1.00 | 29.26 |
| ATOM | 9636 | CA | SER | B | 473 | 74.212 | 61.325 | 87.795 | 1.00 | 29.27 |
| ATOM | 9637 | C | SER | B | 473 | 75.397 | 60.566 | 87.258 | 1.00 | 27.53 |
| ATOM | 9638 | O | SER | B | 473 | 75.282 | 59.961 | 86.221 | 1.00 | 27.50 |
| ATOM | 9639 | CB | SER | B | 473 | 73.731 | 62.245 | 86.684 | 1.00 | 28.76 |
| ATOM | 9640 | OG | SER | B | 473 | 72.382 | 62.487 | 86.896 | 1.00 | 32.10 |
| ATOM | 9641 | N | GLY | B | 474 | 76.525 | 60.578 | 87.943 | 1.00 | 26.50 |
| ATOM | 9642 | CA | GLY | B | 474 | 77.666 | 59.806 | 87.503 | 1.00 | 25.47 |
| ATOM | 9643 | C | GLY | B | 474 | 78.932 | 60.365 | 88.098 | 1.00 | 25.87 |
| ATOM | 9644 | O | GLY | B | 474 | 78.846 | 61.403 | 88.770 | 1.00 | 25.00 |
| ATOM | 9645 | N | PRO | B | 475 | 80.108 | 59.796 | 87.778 | 1.00 | 26.49 |
| ATOM | 9646 | CA | PRO | B | 475 | 80.259 | 58.688 | 86.820 | 1.00 | 26.73 |
| ATOM | 9647 | C | PRO | B | 475 | 79.769 | 57.375 | 87.280 | 1.00 | 27.39 |
| ATOM | 9648 | O | PRO | B | 475 | 79.668 | 56.558 | 86.405 | 1.00 | 26.57 |
| ATOM | 9649 | CB | PRO | B | 475 | 81.782 | 58.574 | 86.624 | 1.00 | 27.70 |
| ATOM | 9650 | CG | PRO | B | 475 | 82.343 | 59.123 | 87.911 | 1.00 | 26.66 |
| ATOM | 9651 | CD | PRO | B | 475 | 81.427 | 60.304 | 88.212 | 1.00 | 26.62 |
| ATOM | 9652 | N | GLY | B | 476 | 79.483 | 57.177 | 88.578 | 1.00 | 27.70 |
| ATOM | 9653 | CA | GLY | B | 476 | 78.979 | 55.898 | 89.060 | 1.00 | 26.91 |
| ATOM | 9654 | C | GLY | B | 476 | 77.468 | 55.857 | 88.895 | 1.00 | 27.17 |
| ATOM | 9655 | O | GLY | B | 476 | 76.858 | 56.716 | 88.201 | 1.00 | 26.20 |
| ATOM | 9656 | N | LEU | B | 477 | 76.854 | 54.849 | 89.489 | 1.00 | 27.44 |
| ATOM | 9657 | CA | LEU | B | 477 | 75.414 | 54.730 | 89.460 | 1.00 | 29.14 |
| ATOM | 9658 | C | LEU | B | 477 | 74.786 | 55.907 | 90.179 | 1.00 | 29.25 |
| ATOM | 9659 | O | LEU | B | 477 | 75.281 | 56.352 | 91.229 | 1.00 | 28.98 |
| ATOM | 9660 | CB | LEU | B | 477 | 74.986 | 53.436 | 90.149 | 1.00 | 29.87 |
| ATOM | 9661 | CG | LEU | B | 477 | 75.705 | 52.213 | 89.586 | 1.00 | 33.15 |
| ATOM | 9662 | CD1 | LEU | B | 477 | 75.080 | 50.971 | 90.149 | 1.00 | 35.05 |
| ATOM | 9663 | CD2 | LEU | B | 477 | 75.645 | 52.212 | 88.035 | 1.00 | 35.43 |
| ATOM | 9664 | N | PRO | B | 478 | 73.711 | 56.434 | 89.610 | 1.00 | 29.49 |
| ATOM | 9665 | CA | PRO | B | 478 | 72.984 | 57.552 | 90.226 | 1.00 | 28.80 |
| ATOM | 9666 | C | PRO | B | 478 | 72.717 | 57.345 | 91.708 | 1.00 | 28.27 |
| ATOM | 9667 | O | PRO | B | 478 | 72.384 | 56.230 | 92.132 | 1.00 | 28.59 |
| ATOM | 9668 | CB | PRO | B | 478 | 71.673 | 57.577 | 89.432 | 1.00 | 28.93 |
| ATOM | 9669 | CG | PRO | B | 478 | 72.099 | 57.117 | 88.030 | 1.00 | 29.83 |
| ATOM | 9670 | CD | PRO | B | 478 | 73.115 | 56.029 | 88.325 | 1.00 | 29.62 |
| ATOM | 9671 | N | LEU | B | 479 | 72.806 | 58.431 | 92.462 | 1.00 | 27.47 |
| ATOM | 9672 | CA | LEU | B | 479 | 72.659 | 58.444 | 93.903 | 1.00 | 27.62 |
| ATOM | 9673 | C | LEU | B | 479 | 71.794 | 59.627 | 94.339 | 1.00 | 26.96 |
| ATOM | 9674 | O | LEU | B | 479 | 72.108 | 60.779 | 94.071 | 1.00 | 25.61 |
| ATOM | 9675 | CB | LEU | B | 479 | 74.048 | 58.628 | 94.544 | 1.00 | 28.55 |
| ATOM | 9676 | CG | LEU | B | 479 | 74.281 | 57.974 | 95.893 | 1.00 | 31.83 |
| ATOM | 9677 | CD1 | LEU | B | 479 | 75.361 | 58.642 | 96.719 | 1.00 | 33.40 |
| ATOM | 9678 | CD2 | LEU | B | 479 | 73.037 | 57.890 | 96.694 | 1.00 | 33.81 |
| ATOM | 9679 | N | TYR | B | 480 | 70.711 | 59.355 | 95.023 | 1.00 | 26.74 |
| ATOM | 9680 | CA | TYR | B | 480 | 69.848 | 60.420 | 95.438 | 1.00 | 27.84 |
| ATOM | 9681 | C | TYR | B | 480 | 69.858 | 60.524 | 96.971 | 1.00 | 27.91 |
| ATOM | 9682 | O | TYR | B | 480 | 69.579 | 59.543 | 97.693 | 1.00 | 28.32 |
| ATOM | 9683 | CB | TYR | B | 480 | 68.423 | 60.190 | 94.938 | 1.00 | 28.47 |
| ATOM | 9684 | CG | TYR | B | 480 | 68.258 | 60.043 | 93.408 | 1.00 | 28.87 |
| ATOM | 9685 | CD1 | TYR | B | 480 | 68.722 | 58.922 | 92.727 | 1.00 | 29.58 |
| ATOM | 9686 | CD2 | TYR | B | 480 | 67.591 | 61.015 | 92.679 | 1.00 | 30.14 |
| ATOM | 9687 | CE1 | TYR | B | 480 | 68.538 | 58.785 | 91.304 | 1.00 | 29.30 |
| ATOM | 9688 | CE2 | TYR | B | 480 | 67.423 | 60.911 | 91.295 | 1.00 | 31.04 |
| ATOM | 9689 | CZ | TYR | B | 480 | 67.909 | 59.797 | 90.613 | 1.00 | 30.37 |
| ATOM | 9690 | OH | TYR | B | 480 | 67.703 | 59.712 | 89.257 | 1.00 | 32.11 |
| ATOM | 9691 | N | THR | B | 481 | 70.133 | 61.717 | 97.460 | 1.00 | 27.62 |
| ATOM | 9692 | CA | THR | B | 481 | 70.252 | 61.928 | 98.888 | 1.00 | 28.14 |
| ATOM | 9693 | C | THR | B | 481 | 69.454 | 63.124 | 99.348 | 1.00 | 28.12 |
| ATOM | 9694 | O | THR | B | 481 | 69.095 | 64.012 | 98.546 | 1.00 | 26.65 |
| ATOM | 9695 | CB | THR | B | 481 | 71.713 | 62.164 | 99.225 | 1.00 | 28.73 |
| ATOM | 9696 | OG1 | THR | B | 481 | 72.253 | 63.151 | 98.338 | 1.00 | 29.12 |
| ATOM | 9697 | CG2 | THR | B | 481 | 72.567 | 60.907 | 98.947 | 1.00 | 28.66 |
| ATOM | 9698 | N | LEU | B | 482 | 69.180 | 63.137 | 100.649 | 1.00 | 28.65 |
| ATOM | 9699 | CA | LEU | B | 482 | 68.458 | 64.229 | 101.282 | 1.00 | 28.48 |
| ATOM | 9700 | C | LEU | B | 482 | 69.356 | 64.914 | 102.280 | 1.00 | 29.11 |
| ATOM | 9701 | O | LEU | B | 482 | 70.196 | 64.291 | 102.924 | 1.00 | 28.32 |
| ATOM | 9702 | CB | LEU | B | 482 | 67.177 | 63.752 | 101.924 | 1.00 | 28.28 |
| ATOM | 9703 | CG | LEU | B | 482 | 66.102 | 64.833 | 102.044 | 1.00 | 28.91 |
| ATOM | 9704 | CD1 | LEU | B | 482 | 65.650 | 65.377 | 100.720 | 1.00 | 26.67 |
| ATOM | 9705 | CD2 | LEU | B | 482 | 64.906 | 64.302 | 102.821 | 1.00 | 27.26 |
| ATOM | 9706 | N | HIS | B | 483 | 69.178 | 66.229 | 102.397 | 1.00 | 29.12 |
| ATOM | 9707 | CA | HIS | B | 483 | 70.073 | 67.026 | 103.203 | 1.00 | 28.26 |
| ATOM | 9708 | C | HIS | B | 483 | 69.396 | 68.181 | 103.927 | 1.00 | 28.91 |
| ATOM | 9709 | O | HIS | B | 483 | 68.454 | 68.801 | 103.416 | 1.00 | 27.89 |
| ATOM | 9710 | CB | HIS | B | 483 | 71.131 | 67.637 | 102.290 | 1.00 | 28.44 |
| ATOM | 9711 | CG | HIS | B | 483 | 72.123 | 66.657 | 101.762 | 1.00 | 27.09 |
| ATOM | 9712 | ND1 | HIS | B | 483 | 71.965 | 66.011 | 100.548 | 1.00 | 29.70 |
| ATOM | 9713 | CD2 | HIS | B | 483 | 73.265 | 66.179 | 102.303 | 1.00 | 27.79 |
| ATOM | 9714 | CE1 | HIS | B | 483 | 72.987 | 65.191 | 100.362 | 1.00 | 30.77 |
| ATOM | 9715 | NE2 | HIS | B | 483 | 73.793 | 65.278 | 101.408 | 1.00 | 26.04 |
| ATOM | 9716 | N | SER | B | 484 | 69.944 | 68.538 | 105.085 | 1.00 | 29.00 |
| ATOM | 9717 | CA | SER | B | 484 | 69.468 | 69.698 | 105.842 | 1.00 | 29.39 |
| ATOM | 9718 | C | SER | B | 484 | 70.397 | 70.903 | 105.664 | 1.00 | 29.47 |
| ATOM | 9719 | O | SER | B | 484 | 71.614 | 70.785 | 105.718 | 1.00 | 27.76 |
| ATOM | 9720 | CB | SER | B | 484 | 69.306 | 69.338 | 107.296 | 1.00 | 29.27 |
| ATOM | 9721 | OG | SER | B | 484 | 69.382 | 70.500 | 108.077 | 1.00 | 32.58 |
| ATOM | 9722 | N | SER | B | 485 | 69.807 | 72.066 | 105.400 | 1.00 | 30.49 |
| ATOM | 9723 | CA | SER | B | 485 | 70.572 | 73.268 | 105.094 | 1.00 | 31.59 |
| ATOM | 9724 | C | SER | B | 485 | 71.282 | 73.943 | 106.272 | 1.00 | 33.08 |
| ATOM | 9725 | O | SER | B | 485 | 72.350 | 74.536 | 106.096 | 1.00 | 32.69 |
| ATOM | 9726 | CB | SER | B | 485 | 69.661 | 74.289 | 104.418 | 1.00 | 31.58 |
| ATOM | 9727 | OG | SER | B | 485 | 69.465 | 73.987 | 103.049 | 1.00 | 31.48 |
| ATOM | 9728 | N | VAL | B | 486 | 70.729 | 73.850 | 107.462 | 1.00 | 34.59 |
| ATOM | 9729 | CA | VAL | B | 486 | 71.284 | 74.659 | 108.534 | 1.00 | 36.93 |
| ATOM | 9730 | C | VAL | B | 486 | 72.762 | 74.420 | 108.654 | 1.00 | 36.73 |
| ATOM | 9731 | O | VAL | B | 486 | 73.536 | 75.376 | 108.644 | 1.00 | 37.87 |
| ATOM | 9732 | CB | VAL | B | 486 | 70.646 | 74.455 | 109.933 | 1.00 | 38.37 |
| ATOM | 9733 | CG1 | VAL | B | 486 | 70.631 | 75.824 | 110.673 | 1.00 | 40.52 |
| ATOM | 9734 | CG2 | VAL | B | 486 | 69.283 | 73.863 | 109.863 | 1.00 | 37.95 |
| ATOM | 9735 | N | ASN | B | 487 | 73.149 | 73.163 | 108.782 | 1.00 | 36.88 |
| ATOM | 9736 | CA | ASN | B | 487 | 74.559 | 72.803 | 108.783 | 1.00 | 37.65 |
| ATOM | 9737 | C | ASN | B | 487 | 74.925 | 71.855 | 107.656 | 1.00 | 37.06 |
| ATOM | 9738 | O | ASN | B | 487 | 75.924 | 71.176 | 107.720 | 1.00 | 36.28 |
| ATOM | 9739 | CB | ASN | B | 487 | 74.953 | 72.198 | 110.124 | 1.00 | 38.31 |
| ATOM | 9740 | CG | ASN | B | 487 | 75.105 | 73.257 | 111.203 | 1.00 | 42.08 |
| ATOM | 9741 | OD1 | ASN | B | 487 | 74.366 | 73.264 | 112.190 | 1.00 | 45.04 |
| ATOM | 9742 | ND2 | ASN | B | 487 | 76.044 | 74.175 | 111.002 | 1.00 | 44.16 |
| ATOM | 9743 | N | ASP | B | 488 | 74.104 | 71.813 | 106.621 | 1.00 | 37.01 |
| ATOM | 9744 | CA | ASP | B | 488 | 74.351 | 70.945 | 105.488 | 1.00 | 36.95 |
| ATOM | 9745 | C | ASP | B | 488 | 74.672 | 69.537 | 105.924 | 1.00 | 36.35 |
| ATOM | 9746 | O | ASP | B | 488 | 75.630 | 68.959 | 105.452 | 1.00 | 36.59 |
| ATOM | 9747 | CB | ASP | B | 488 | 75.479 | 71.483 | 104.624 | 1.00 | 36.37 |
| ATOM | 9748 | CG | ASP | B | 488 | 75.113 | 72.793 | 103.972 | 1.00 | 36.30 |
| ATOM | 9749 | OD1 | ASP | B | 488 | 74.391 | 72.777 | 102.954 | 1.00 | 32.54 |
| ATOM | 9750 | OD2 | ASP | B | 488 | 75.479 | 73.886 | 104.423 | 1.00 | 34.83 |
| ATOM | 9751 | N | LYS | B | 489 | 73.878 | 68.995 | 106.828 | 1.00 | 35.78 |
| ATOM | 9752 | CA | LYS | B | 489 | 74.103 | 67.639 | 107.249 | 1.00 | 36.75 |
| ATOM | 9753 | C | LYS | B | 489 | 73.393 | 66.684 | 106.292 | 1.00 | 35.41 |
| ATOM | 9754 | O | LYS | B | 489 | 72.326 | 67.022 | 105.761 | 1.00 | 33.88 |
| ATOM | 9755 | CB | LYS | B | 489 | 73.583 | 67.402 | 108.665 | 1.00 | 37.41 |
| ATOM | 9756 | CG | LYS | B | 489 | 73.970 | 66.006 | 109.152 | 1.00 | 43.10 |
| ATOM | 9757 | CD | LYS | B | 489 | 73.914 | 65.802 | 110.666 | 1.00 | 47.45 |
| ATOM | 9758 | CE | LYS | B | 489 | 74.643 | 64.498 | 111.029 | 1.00 | 49.75 |
| ATOM | 9759 | NZ | LYS | B | 489 | 73.966 | 63.784 | 112.162 | 1.00 | 52.01 |
| ATOM | 9760 | N | GLY | B | 490 | 74.010 | 65.519 | 106.069 | 1.00 | 33.74 |
| ATOM | 9761 | CA | GLY | B | 490 | 73.389 | 64.429 | 105.323 | 1.00 | 33.26 |
| ATOM | 9762 | C | GLY | B | 490 | 72.260 | 63.858 | 106.173 | 1.00 | 32.76 |
| ATOM | 9763 | O | GLY | B | 490 | 72.438 | 63.624 | 107.347 | 1.00 | 32.36 |
| ATOM | 9764 | N | LEU | B | 491 | 71.055 | 63.734 | 105.636 | 1.00 | 33.01 |
| ATOM | 9765 | CA | LEU | B | 491 | 69.974 | 63.157 | 106.427 | 1.00 | 33.73 |
| ATOM | 9766 | C | LEU | B | 491 | 69.923 | 61.654 | 106.250 | 1.00 | 34.58 |
| ATOM | 9767 | O | LEU | B | 491 | 69.950 | 60.918 | 107.206 | 1.00 | 32.32 |
| ATOM | 9768 | CB | LEU | B | 491 | 68.624 | 63.745 | 106.026 | 1.00 | 33.78 |
| ATOM | 9769 | CG | LEU | B | 491 | 68.517 | 65.161 | 106.584 | 1.00 | 36.24 |
| ATOM | 9770 | CD1 | LEU | B | 491 | 67.357 | 65.934 | 105.994 | 1.00 | 37.30 |
| ATOM | 9771 | CD2 | LEU | B | 491 | 68.376 | 65.069 | 108.117 | 1.00 | 38.67 |
| ATOM | 9772 | N | ARG | B | 492 | 69.904 | 61.234 | 104.982 | 1.00 | 35.31 |
| ATOM | 9773 | CA | ARG | B | 492 | 69.635 | 59.865 | 104.603 | 1.00 | 36.20 |
| ATOM | 9774 | C | ARG | B | 492 | 70.301 | 59.591 | 103.277 | 1.00 | 36.65 |
| ATOM | 9775 | O | ARG | B | 492 | 71.191 | 60.333 | 102.829 | 1.00 | 42.14 |
| ATOM | 9776 | CB | ARG | B | 492 | 68.153 | 59.765 | 104.305 | 1.00 | 36.71 |
| ATOM | 9777 | CG | ARG | B | 492 | 67.302 | 59.179 | 105.321 | 1.00 | 38.38 |
| ATOM | 9778 | CD | ARG | B | 492 | 65.846 | 59.766 | 105.368 | 1.00 | 37.82 |
| ATOM | 9779 | NE | ARG | B | 492 | 65.740 | 60.571 | 106.570 | 1.00 | 37.08 |
| ATOM | 9780 | CZ | ARG | B | 492 | 65.113 | 61.712 | 106.676 | 1.00 | 36.90 |
| ATOM | 9781 | NH1 | ARG | B | 492 | 64.458 | 62.231 | 105.651 | 1.00 | 37.47 |
| ATOM | 9782 | NH2 | ARG | B | 492 | 65.141 | 62.343 | 107.836 | 1.00 | 37.23 |
| ATOM | 9783 | N | VAL | B | 493 | 69.743 | 58.579 | 102.634 | 1.00 | 34.01 |
| ATOM | 9784 | CA | VAL | B | 493 | 70.026 | 58.130 | 101.285 | 1.00 | 31.94 |
| ATOM | 9785 | C | VAL | B | 493 | 68.606 | 57.792 | 100.799 | 1.00 | 30.58 |
| ATOM | 9786 | O | VAL | B | 493 | 67.872 | 57.111 | 101.479 | 1.00 | 29.50 |
| ATOM | 9787 | CB | VAL | B | 493 | 70.865 | 56.823 | 101.237 | 1.00 | 32.57 |
| ATOM | 9788 | CG1 | VAL | B | 493 | 70.785 | 56.170 | 99.856 | 1.00 | 32.60 |
| ATOM | 9789 | CG2 | VAL | B | 493 | 72.359 | 57.060 | 101.638 | 1.00 | 30.30 |
| ATOM | 9790 | N | LEU | B | 494 | 68.182 | 58.335 | 99.668 | 1.00 | 29.23 |
| ATOM | 9791 | CA | LEU | B | 494 | 66.841 | 58.051 | 99.168 | 1.00 | 28.90 |
| ATOM | 9792 | C | LEU | B | 494 | 66.799 | 56.875 | 98.178 | 1.00 | 28.74 |
| ATOM | 9793 | O | LEU | B | 494 | 65.873 | 56.074 | 98.206 | 1.00 | 28.66 |
| ATOM | 9794 | CB | LEU | B | 494 | 66.238 | 59.316 | 98.529 | 1.00 | 28.87 |
| ATOM | 9795 | CG | LEU | B | 494 | 66.174 | 60.493 | 99.513 | 1.00 | 28.66 |
| ATOM | 9796 | CD1 | LEU | B | 494 | 66.005 | 61.778 | 98.767 | 1.00 | 30.43 |
| ATOM | 9797 | CD2 | LEU | B | 494 | 65.057 | 60.283 | 100.491 | 1.00 | 28.40 |
| ATOM | 9798 | N | GLU | B | 495 | 67.786 | 56.795 | 97.302 | 1.00 | 28.15 |
| ATOM | 9799 | CA | GLU | B | 495 | 67.887 | 55.707 | 96.379 | 1.00 | 27.95 |
| ATOM | 9800 | C | GLU | B | 495 | 69.337 | 55.586 | 95.978 | 1.00 | 28.14 |
| ATOM | 9801 | O | GLU | B | 495 | 69.961 | 56.580 | 95.595 | 1.00 | 27.78 |
| ATOM | 9802 | CB | GLU | B | 495 | 67.032 | 55.973 | 95.150 | 1.00 | 28.63 |
| ATOM | 9803 | CG | GLU | B | 495 | 67.354 | 55.054 | 93.983 | 1.00 | 28.25 |
| ATOM | 9804 | CD | GLU | B | 495 | 66.934 | 53.629 | 94.279 | 1.00 | 27.99 |
| ATOM | 9805 | OE1 | GLU | B | 495 | 65.762 | 53.446 | 94.588 | 1.00 | 28.05 |
| ATOM | 9806 | OE2 | GLU | B | 495 | 67.775 | 52.715 | 94.225 | 1.00 | 29.19 |
| ATOM | 9807 | N | ASP | B | 496 | 69.892 | 54.384 | 96.073 | 1.00 | 28.51 |
| ATOM | 9808 | CA | ASP | B | 496 | 71.311 | 54.212 | 95.778 | 1.00 | 29.15 |
| ATOM | 9809 | C | ASP | B | 496 | 71.593 | 53.254 | 94.667 | 1.00 | 29.08 |
| ATOM | 9810 | O | ASP | B | 496 | 72.753 | 52.957 | 94.382 | 1.00 | 28.40 |
| ATOM | 9811 | CB | ASP | B | 496 | 72.096 | 53.812 | 97.023 | 1.00 | 29.89 |
| ATOM | 9812 | CG | ASP | B | 496 | 71.724 | 52.460 | 97.552 | 1.00 | 31.02 |
| ATOM | 9813 | OD1 | ASP | B | 496 | 71.067 | 51.659 | 96.856 | 1.00 | 35.67 |
| ATOM | 9814 | OD2 | ASP | B | 496 | 72.039 | 52.119 | 98.687 | 1.00 | 34.89 |
| ATOM | 9815 | N | ASN | B | 497 | 70.517 | 52.797 | 94.026 | 1.00 | 30.29 |
| ATOM | 9816 | CA | ASN | B | 497 | 70.582 | 51.889 | 92.893 | 1.00 | 29.79 |
| ATOM | 9817 | C | ASN | B | 497 | 71.361 | 50.576 | 93.166 | 1.00 | 30.49 |
| ATOM | 9818 | O | ASN | B | 497 | 71.994 | 50.019 | 92.256 | 1.00 | 30.08 |
| ATOM | 9819 | CB | ASN | B | 497 | 71.171 | 52.639 | 91.702 | 1.00 | 30.35 |
| ATOM | 9820 | CG | ASN | B | 497 | 70.102 | 53.332 | 90.851 | 1.00 | 30.90 |
| ATOM | 9821 | OD1 | ASN | B | 497 | 69.234 | 52.671 | 90.257 | 1.00 | 32.35 |
| ATOM | 9822 | ND2 | ASN | B | 497 | 70.201 | 54.647 | 90.732 | 1.00 | 30.45 |
| ATOM | 9823 | N | SER | B | 498 | 71.332 | 50.098 | 94.415 | 1.00 | 31.45 |
| ATOM | 9824 | CA | SER | B | 498 | 71.999 | 48.843 | 94.787 | 1.00 | 32.80 |
| ATOM | 9825 | C | SER | B | 498 | 71.441 | 47.651 | 93.976 | 1.00 | 33.07 |
| ATOM | 9826 | O | SER | B | 498 | 72.184 | 46.767 | 93.603 | 1.00 | 32.06 |
| ATOM | 9827 | CB | SER | B | 498 | 71.924 | 48.571 | 96.304 | 1.00 | 33.14 |
| ATOM | 9828 | OG | SER | B | 498 | 70.582 | 48.684 | 96.811 | 1.00 | 35.98 |
| ATOM | 9829 | N | ALA | B | 499 | 70.158 | 47.673 | 93.627 | 1.00 | 33.72 |
| ATOM | 9830 | CA | ALA | B | 499 | 69.624 | 46.581 | 92.847 | 1.00 | 34.12 |
| ATOM | 9831 | C | ALA | B | 499 | 70.327 | 46.512 | 91.515 | 1.00 | 34.76 |
| ATOM | 9832 | O | ALA | B | 499 | 70.775 | 45.437 | 91.109 | 1.00 | 34.90 |
| ATOM | 9833 | CB | ALA | B | 499 | 68.133 | 46.713 | 92.645 | 1.00 | 34.02 |
| ATOM | 9834 | N | LEU | B | 500 | 70.457 | 47.660 | 90.848 | 1.00 | 35.42 |
| ATOM | 9835 | CA | LEU | B | 500 | 71.099 | 47.714 | 89.528 | 1.00 | 35.59 |
| ATOM | 9836 | C | LEU | B | 500 | 72.545 | 47.234 | 89.678 | 1.00 | 35.28 |
| ATOM | 9837 | O | LEU | B | 500 | 73.070 | 46.446 | 88.882 | 1.00 | 34.53 |
| ATOM | 9838 | CB | LEU | B | 500 | 71.062 | 49.159 | 88.981 | 1.00 | 34.97 |
| ATOM | 9839 | CG | LEU | B | 500 | 71.027 | 49.422 | 87.471 | 1.00 | 37.81 |
| ATOM | 9840 | CD1 | LEU | B | 500 | 71.798 | 50.707 | 87.044 | 1.00 | 37.23 |
| ATOM | 9841 | CD2 | LEU | B | 500 | 71.501 | 48.241 | 86.654 | 1.00 | 36.80 |
| ATOM | 9842 | N | ASP | B | 501 | 73.167 | 47.709 | 90.734 | 1.00 | 35.54 |
| ATOM | 9843 | CA | ASP | B | 501 | 74.569 | 47.395 | 91.038 | 1.00 | 36.79 |
| ATOM | 9844 | C | ASP | B | 501 | 74.796 | 45.879 | 91.053 | 1.00 | 36.68 |
| ATOM | 9845 | O | ASP | B | 501 | 75.735 | 45.371 | 90.410 | 1.00 | 34.68 |
| ATOM | 9846 | CB | ASP | B | 501 | 74.939 | 48.008 | 92.403 | 1.00 | 37.28 |
| ATOM | 9847 | CG | ASP | B | 501 | 76.433 | 47.924 | 92.714 | 1.00 | 39.44 |
| ATOM | 9848 | OD1 | ASP | B | 501 | 77.265 | 48.026 | 91.803 | 1.00 | 39.64 |
| ATOM | 9849 | OD2 | ASP | B | 501 | 76.866 | 47.768 | 93.877 | 1.00 | 47.25 |
| ATOM | 9850 | N | LYS | B | 502 | 73.898 | 45.171 | 91.744 | 1.00 | 37.33 |
| ATOM | 9851 | CA | LYS | B | 502 | 73.987 | 43.718 | 91.841 | 1.00 | 39.36 |
| ATOM | 9852 | C | LYS | B | 502 | 73.823 | 43.087 | 90.464 | 1.00 | 38.86 |
| ATOM | 9853 | O | LYS | B | 502 | 74.649 | 42.281 | 90.098 | 1.00 | 37.72 |
| ATOM | 9854 | CB | LYS | B | 502 | 73.046 | 43.111 | 92.925 | 1.00 | 39.93 |
| ATOM | 9855 | CG | LYS | B | 502 | 73.774 | 43.104 | 94.326 | 1.00 | 44.28 |
| ATOM | 9856 | CD | LYS | B | 502 | 72.979 | 42.616 | 95.592 | 1.00 | 50.35 |
| ATOM | 9857 | CE | LYS | B | 502 | 73.659 | 43.132 | 96.928 | 1.00 | 52.43 |
| ATOM | 9858 | NZ | LYS | B | 502 | 73.915 | 42.088 | 98.002 | 1.00 | 52.50 |
| ATOM | 9859 | N | MET | B | 503 | 72.807 | 43.489 | 89.699 | 1.00 | 38.71 |
| ATOM | 9860 | CA | MET | B | 503 | 72.674 | 42.995 | 88.322 | 1.00 | 39.36 |
| ATOM | 9861 | C | MET | B | 503 | 73.907 | 43.319 | 87.458 | 1.00 | 38.16 |
| ATOM | 9862 | O | MET | B | 503 | 74.358 | 42.498 | 86.698 | 1.00 | 38.26 |
| ATOM | 9863 | CB | MET | B | 503 | 71.441 | 43.584 | 87.651 | 1.00 | 39.98 |
| ATOM | 9864 | CG | MET | B | 503 | 70.136 | 42.959 | 88.096 | 1.00 | 44.31 |
| ATOM | 9865 | SD | MET | B | 503 | 68.772 | 43.508 | 87.082 | 1.00 | 51.44 |
| ATOM | 9866 | CE | MET | B | 503 | 68.643 | 45.197 | 87.624 | 1.00 | 52.23 |
| ATOM | 9867 | N | LEU | B | 504 | 74.492 | 44.497 | 87.592 | 1.00 | 38.11 |
| ATOM | 9868 | CA | LEU | B | 504 | 75.578 | 44.868 | 86.672 | 1.00 | 37.49 |
| ATOM | 9869 | C | LEU | B | 504 | 76.902 | 44.113 | 86.877 | 1.00 | 38.02 |
| ATOM | 9870 | O | LEU | B | 504 | 77.662 | 43.874 | 85.915 | 1.00 | 36.62 |
| ATOM | 9871 | CB | LEU | B | 504 | 75.778 | 46.381 | 86.679 | 1.00 | 36.89 |
| ATOM | 9872 | CG | LEU | B | 504 | 74.661 | 47.111 | 85.894 | 1.00 | 36.85 |
| ATOM | 9873 | CD1 | LEU | B | 504 | 74.774 | 48.625 | 86.012 | 1.00 | 36.54 |
| ATOM | 9874 | CD2 | LEU | B | 504 | 74.606 | 46.721 | 84.398 | 1.00 | 36.84 |
| ATOM | 9875 | N | GLN | B | 505 | 77.151 | 43.715 | 88.125 | 1.00 | 38.71 |
| ATOM | 9876 | CA | GLN | B | 505 | 78.328 | 42.934 | 88.458 | 1.00 | 39.81 |
| ATOM | 9877 | C | GLN | B | 505 | 78.379 | 41.669 | 87.594 | 1.00 | 39.33 |
| ATOM | 9878 | O | GLN | B | 505 | 79.428 | 41.163 | 87.366 | 1.00 | 37.85 |
| ATOM | 9879 | CB | GLN | B | 505 | 78.398 | 42.623 | 89.983 | 1.00 | 40.66 |
| ATOM | 9880 | CG | GLN | B | 505 | 78.882 | 43.837 | 90.863 | 1.00 | 43.70 |
| ATOM | 9881 | CD | GLN | B | 505 | 78.675 | 43.624 | 92.373 | 1.00 | 48.71 |
| ATOM | 9882 | OE1 | GLN | B | 505 | 78.565 | 42.483 | 92.828 | 1.00 | 53.18 |
| ATOM | 9883 | NE2 | GLN | B | 505 | 78.609 | 44.725 | 93.148 | 1.00 | 50.14 |
| ATOM | 9884 | N | ASN | B | 506 | 77.238 | 41.162 | 87.126 | 1.00 | 40.26 |
| ATOM | 9885 | CA | ASN | B | 506 | 77.246 | 40.027 | 86.185 | 1.00 | 40.96 |
| ATOM | 9886 | C | ASN | B | 506 | 77.392 | 40.357 | 84.692 | 1.00 | 39.89 |
| ATOM | 9887 | O | ASN | B | 506 | 77.086 | 39.526 | 83.856 | 1.00 | 41.00 |
| ATOM | 9888 | CB | ASN | B | 506 | 75.991 | 39.186 | 86.344 | 1.00 | 41.01 |
| ATOM | 9889 | CG | ASN | B | 506 | 75.866 | 38.596 | 87.737 | 1.00 | 44.86 |
| ATOM | 9890 | OD1 | ASN | B | 506 | 76.847 | 38.085 | 88.280 | 1.00 | 47.76 |
| ATOM | 9891 | ND2 | ASN | B | 506 | 74.660 | 38.685 | 88.336 | 1.00 | 45.29 |
| ATOM | 9892 | N | VAL | B | 507 | 77.846 | 41.541 | 84.326 | 1.00 | 39.73 |
| ATOM | 9893 | CA | VAL | B | 507 | 77.972 | 41.834 | 82.899 | 1.00 | 39.38 |
| ATOM | 9894 | C | VAL | B | 507 | 79.276 | 42.499 | 82.603 | 1.00 | 38.86 |
| ATOM | 9895 | O | VAL | B | 507 | 79.876 | 43.126 | 83.464 | 1.00 | 38.82 |
| ATOM | 9896 | CB | VAL | B | 507 | 76.728 | 42.619 | 82.312 | 1.00 | 39.16 |
| ATOM | 9897 | CG1 | VAL | B | 507 | 76.185 | 43.507 | 83.274 | 1.00 | 39.25 |
| ATOM | 9898 | CG2 | VAL | B | 507 | 77.074 | 43.370 | 81.001 | 1.00 | 39.86 |
| ATOM | 9899 | N | GLN | B | 508 | 79.764 | 42.299 | 81.397 | 1.00 | 38.32 |
| ATOM | 9900 | CA | GLN | B | 508 | 81.007 | 42.929 | 81.008 | 1.00 | 38.75 |
| ATOM | 9901 | C | GLN | B | 508 | 80.707 | 44.357 | 80.558 | 1.00 | 38.50 |
| ATOM | 9902 | O | GLN | B | 508 | 80.597 | 44.636 | 79.384 | 1.00 | 38.85 |
| ATOM | 9903 | CB | GLN | B | 508 | 81.712 | 42.146 | 79.904 | 1.00 | 38.76 |
| ATOM | 9904 | CG | GLN | B | 508 | 81.790 | 40.648 | 80.165 | 1.00 | 39.75 |
| ATOM | 9905 | CD | GLN | B | 508 | 82.822 | 39.949 | 79.313 | 1.00 | 38.27 |
| ATOM | 9906 | OE1 | GLN | B | 508 | 83.768 | 40.560 | 78.850 | 1.00 | 39.71 |
| ATOM | 9907 | NE2 | GLN | B | 508 | 82.637 | 38.668 | 79.114 | 1.00 | 38.73 |
| ATOM | 9908 | N | MET | B | 509 | 80.585 | 45.246 | 81.527 | 1.00 | 38.00 |
| ATOM | 9909 | CA | MET | B | 509 | 80.310 | 46.635 | 81.267 | 1.00 | 37.57 |
| ATOM | 9910 | C | MET | B | 509 | 81.524 | 47.344 | 80.710 | 1.00 | 36.16 |
| ATOM | 9911 | O | MET | B | 509 | 82.628 | 47.031 | 81.051 | 1.00 | 36.25 |
| ATOM | 9912 | CB | MET | B | 509 | 79.876 | 47.311 | 82.560 | 1.00 | 37.43 |
| ATOM | 9913 | CG | MET | B | 509 | 78.539 | 46.824 | 83.006 | 1.00 | 38.53 |
| ATOM | 9914 | SD | MET | B | 509 | 77.297 | 47.127 | 81.754 | 1.00 | 42.19 |
| ATOM | 9915 | CE | MET | B | 509 | 77.117 | 48.812 | 81.941 | 1.00 | 41.34 |
| ATOM | 9916 | N | PRO | B | 510 | 81.300 | 48.300 | 79.831 | 1.00 | 34.57 |
| ATOM | 9917 | CA | PRO | B | 510 | 82.376 | 49.102 | 79.288 | 1.00 | 33.86 |
| ATOM | 9918 | C | PRO | B | 510 | 82.774 | 50.098 | 80.333 | 1.00 | 32.76 |
| ATOM | 9919 | O | PRO | B | 510 | 82.014 | 50.241 | 81.244 | 1.00 | 31.08 |
| ATOM | 9920 | CB | PRO | B | 510 | 81.700 | 49.901 | 78.187 | 1.00 | 33.77 |
| ATOM | 9921 | CG | PRO | B | 510 | 80.316 | 49.835 | 78.421 | 1.00 | 33.46 |
| ATOM | 9922 | CD | PRO | B | 510 | 79.990 | 48.710 | 79.336 | 1.00 | 34.56 |
| ATOM | 9923 | N | SER | B | 511 | 83.899 | 50.774 | 80.166 | 1.00 | 32.79 |
| ATOM | 9924 | CA | SER | B | 511 | 84.280 | 51.877 | 81.033 | 1.00 | 33.68 |
| ATOM | 9925 | C | SER | B | 511 | 84.307 | 53.151 | 80.178 | 1.00 | 34.32 |
| ATOM | 9926 | O | SER | B | 511 | 84.276 | 53.084 | 78.955 | 1.00 | 33.49 |
| ATOM | 9927 | CB | SER | B | 511 | 85.686 | 51.678 | 81.567 | 1.00 | 33.29 |
| ATOM | 9928 | OG | SER | B | 511 | 86.548 | 51.743 | 80.467 | 1.00 | 32.77 |
| ATOM | 9929 | N | LYS | B | 512 | 84.486 | 54.284 | 80.852 | 1.00 | 35.20 |
| ATOM | 9930 | CA | LYS | B | 512 | 84.432 | 55.594 | 80.247 | 1.00 | 35.05 |
| ATOM | 9931 | C | LYS | B | 512 | 85.666 | 56.384 | 80.609 | 1.00 | 35.24 |
| ATOM | 9932 | O | LYS | B | 512 | 86.055 | 56.478 | 81.783 | 1.00 | 35.24 |
| ATOM | 9933 | CB | LYS | B | 512 | 83.177 | 56.322 | 80.747 | 1.00 | 35.16 |
| ATOM | 9934 | CG | LYS | B | 512 | 82.882 | 57.666 | 80.076 | 1.00 | 34.89 |
| ATOM | 9935 | CD | LYS | B | 512 | 81.520 | 58.200 | 80.523 | 1.00 | 31.54 |
| ATOM | 9936 | CE | LYS | B | 512 | 81.212 | 59.556 | 79.895 | 1.00 | 31.66 |
| ATOM | 9937 | NZ | LYS | B | 512 | 79.844 | 60.196 | 80.379 | 1.00 | 30.48 |
| ATOM | 9938 | N | LYS | B | 513 | 86.308 | 56.926 | 79.588 | 1.00 | 34.66 |
| ATOM | 9939 | CA | LYS | B | 513 | 87.422 | 57.811 | 79.789 | 1.00 | 34.55 |
| ATOM | 9940 | C | LYS | B | 513 | 86.985 | 59.227 | 79.377 | 1.00 | 33.27 |
| ATOM | 9941 | O | LYS | B | 513 | 86.400 | 59.410 | 78.302 | 1.00 | 32.86 |
| ATOM | 9942 | CB | LYS | B | 513 | 88.582 | 57.355 | 78.914 | 1.00 | 35.28 |
| ATOM | 9943 | CG | LYS | B | 513 | 89.911 | 57.867 | 79.360 | 1.00 | 38.42 |
| ATOM | 9944 | CD | LYS | B | 513 | 90.834 | 58.109 | 78.151 | 1.00 | 42.99 |
| ATOM | 9945 | CE | LYS | B | 513 | 92.356 | 58.129 | 78.533 | 1.00 | 43.66 |
| ATOM | 9946 | NZ | LYS | B | 513 | 93.216 | 58.331 | 77.286 | 1.00 | 45.03 |
| ATOM | 9947 | N | LEU | B | 514 | 87.317 | 60.193 | 80.225 | 1.00 | 31.88 |
| ATOM | 9948 | CA | LEU | B | 514 | 87.053 | 61.623 | 80.103 | 1.00 | 32.15 |
| ATOM | 9949 | C | LEU | B | 514 | 88.411 | 62.317 | 80.196 | 1.00 | 32.80 |
| ATOM | 9950 | O | LEU | B | 514 | 89.101 | 62.203 | 81.224 | 1.00 | 31.05 |
| ATOM | 9951 | CB | LEU | B | 514 | 86.226 | 62.083 | 81.299 | 1.00 | 32.55 |
| ATOM | 9952 | CG | LEU | B | 514 | 85.150 | 63.166 | 81.171 | 1.00 | 35.94 |
| ATOM | 9953 | CD1 | LEU | B | 514 | 84.978 | 63.884 | 82.488 | 1.00 | 35.20 |
| ATOM | 9954 | CD2 | LEU | B | 514 | 85.384 | 64.165 | 80.046 | 1.00 | 37.34 |
| ATOM | 9955 | N | ASP | B | 515 | 88.803 | 63.044 | 79.154 | 1.00 | 33.60 |
| ATOM | 9956 | CA | ASP | B | 515 | 90.157 | 63.628 | 79.091 | 1.00 | 33.94 |
| ATOM | 9957 | C | ASP | B | 515 | 90.149 | 64.734 | 78.048 | 1.00 | 33.67 |
| ATOM | 9958 | O | ASP | B | 515 | 89.094 | 65.114 | 77.563 | 1.00 | 32.82 |
| ATOM | 9959 | CB | ASP | B | 515 | 91.142 | 62.547 | 78.675 | 1.00 | 34.54 |
| ATOM | 9960 | CG | ASP | B | 515 | 92.569 | 62.806 | 79.134 | 1.00 | 37.60 |
| ATOM | 9961 | OD1 | ASP | B | 515 | 92.889 | 63.930 | 79.590 | 1.00 | 37.94 |
| ATOM | 9962 | OD2 | ASP | B | 515 | 93.428 | 61.893 | 79.085 | 1.00 | 39.77 |
| ATOM | 9963 | N | PHE | B | 516 | 91.307 | 65.257 | 77.690 | 1.00 | 34.11 |
| ATOM | 9964 | CA | PHE | B | 516 | 91.348 | 66.335 | 76.720 | 1.00 | 35.27 |
| ATOM | 9965 | C | PHE | B | 516 | 92.548 | 66.256 | 75.791 | 1.00 | 36.20 |
| ATOM | 9966 | O | PHE | B | 516 | 93.502 | 65.580 | 76.089 | 1.00 | 35.24 |
| ATOM | 9967 | CB | PHE | B | 516 | 91.345 | 67.673 | 77.432 | 1.00 | 35.14 |
| ATOM | 9968 | CG | PHE | B | 516 | 92.512 | 67.863 | 78.341 | 1.00 | 37.49 |
| ATOM | 9969 | CD1 | PHE | B | 516 | 93.738 | 68.247 | 77.843 | 1.00 | 37.88 |
| ATOM | 9970 | CD2 | PHE | B | 516 | 92.400 | 67.606 | 79.709 | 1.00 | 37.60 |
| ATOM | 9971 | CE1 | PHE | B | 516 | 94.831 | 68.408 | 78.699 | 1.00 | 39.15 |
| ATOM | 9972 | CE2 | PHE | B | 516 | 93.484 | 67.777 | 80.558 | 1.00 | 37.83 |
| ATOM | 9973 | CZ | PHE | B | 516 | 94.690 | 68.171 | 80.062 | 1.00 | 38.26 |
| ATOM | 9974 | N | ILE | B | 517 | 92.436 | 66.922 | 74.638 | 1.00 | 37.39 |
| ATOM | 9975 | CA | ILE | B | 517 | 93.513 | 67.051 | 73.677 | 1.00 | 38.97 |
| ATOM | 9976 | C | ILE | B | 517 | 93.673 | 68.531 | 73.511 | 1.00 | 39.81 |
| ATOM | 9977 | O | ILE | B | 517 | 92.835 | 69.307 | 73.953 | 1.00 | 39.05 |
| ATOM | 9978 | CB | ILE | B | 517 | 93.203 | 66.452 | 72.271 | 1.00 | 39.15 |
| ATOM | 9979 | CG1 | ILE | B | 517 | 91.788 | 66.798 | 71.825 | 1.00 | 39.44 |
| ATOM | 9980 | CG2 | ILE | B | 517 | 93.393 | 64.974 | 72.266 | 1.00 | 40.32 |
| ATOM | 9981 | CD1 | ILE | B | 517 | 91.429 | 66.206 | 70.534 | 1.00 | 40.76 |
| ATOM | 9982 | N | ILE | B | 518 | 94.752 | 68.900 | 72.846 | 1.00 | 40.72 |
| ATOM | 9983 | CA | ILE | B | 518 | 95.088 | 70.276 | 72.615 | 1.00 | 41.90 |
| ATOM | 9984 | C | ILE | B | 518 | 94.874 | 70.476 | 71.158 | 1.00 | 42.73 |
| ATOM | 9985 | O | ILE | B | 518 | 95.324 | 69.688 | 70.362 | 1.00 | 44.34 |
| ATOM | 9986 | CB | ILE | B | 518 | 96.573 | 70.515 | 72.897 | 1.00 | 42.61 |
| ATOM | 9987 | CG1 | ILE | B | 518 | 96.979 | 69.981 | 74.279 | 1.00 | 41.35 |
| ATOM | 9988 | CG2 | ILE | B | 518 | 96.920 | 71.993 | 72.712 | 1.00 | 43.42 |
| ATOM | 9989 | CD1 | ILE | B | 518 | 96.412 | 70.764 | 75.432 | 1.00 | 40.08 |
| ATOM | 9990 | N | LEU | B | 519 | 94.162 | 71.503 | 70.788 | 1.00 | 43.62 |
| ATOM | 9991 | CA | LEU | B | 519 | 94.045 | 71.842 | 69.387 | 1.00 | 44.63 |
| ATOM | 9992 | C | LEU | B | 519 | 94.407 | 73.301 | 69.372 | 1.00 | 44.70 |
| ATOM | 9993 | O | LEU | B | 519 | 93.726 | 74.111 | 69.973 | 1.00 | 43.61 |
| ATOM | 9994 | CB | LEU | B | 519 | 92.621 | 71.638 | 68.847 | 1.00 | 44.70 |
| ATOM | 9995 | CG | LEU | B | 519 | 92.252 | 70.377 | 68.061 | 1.00 | 46.16 |
| ATOM | 9996 | CD1 | LEU | B | 519 | 92.594 | 69.095 | 68.773 | 1.00 | 47.42 |
| ATOM | 9997 | CD2 | LEU | B | 519 | 90.767 | 70.403 | 67.732 | 1.00 | 46.83 |
| ATOM | 9998 | N | ASN | B | 520 | 95.531 | 73.623 | 68.747 | 1.00 | 45.98 |
| ATOM | 9999 | CA | ASN | B | 520 | 95.927 | 75.005 | 68.595 | 1.00 | 46.60 |
| ATOM | 10000 | C | ASN | B | 520 | 95.971 | 75.772 | 69.875 | 1.00 | 46.05 |
| ATOM | 10001 | O | ASN | B | 520 | 95.267 | 76.770 | 70.030 | 1.00 | 46.58 |
| ATOM | 10002 | CB | ASN | B | 520 | 94.967 | 75.712 | 67.629 | 1.00 | 46.98 |
| ATOM | 10003 | CG | ASN | B | 520 | 95.511 | 75.757 | 66.248 | 1.00 | 49.34 |
| ATOM | 10004 | OD1 | ASN | B | 520 | 96.647 | 75.308 | 66.033 | 1.00 | 51.14 |
| ATOM | 10005 | ND2 | ASN | B | 520 | 94.742 | 76.314 | 65.291 | 1.00 | 49.12 |
| ATOM | 10006 | N | GLU | B | 521 | 96.781 | 75.311 | 70.803 | 1.00 | 45.72 |
| ATOM | 10007 | CA | GLU | B | 521 | 96.954 | 76.038 | 72.054 | 1.00 | 45.79 |
| ATOM | 10008 | C | GLU | B | 521 | 95.832 | 75.864 | 73.051 | 1.00 | 44.04 |
| ATOM | 10009 | O | GLU | B | 521 | 95.960 | 76.346 | 74.189 | 1.00 | 45.10 |
| ATOM | 10010 | CB | GLU | B | 521 | 97.053 | 77.555 | 71.797 | 1.00 | 47.02 |
| ATOM | 10011 | CG | GLU | B | 521 | 98.446 | 78.176 | 71.813 | 1.00 | 50.35 |
| ATOM | 10012 | CD | GLU | B | 521 | 98.397 | 79.707 | 71.986 | 1.00 | 53.77 |
| ATOM | 10013 | OE1 | GLU | B | 521 | 97.369 | 80.234 | 72.479 | 1.00 | 51.60 |
| ATOM | 10014 | OE2 | GLU | B | 521 | 99.397 | 80.385 | 71.635 | 1.00 | 57.13 |
| ATOM | 10015 | N | THR | B | 522 | 94.716 | 75.247 | 72.661 | 1.00 | 41.38 |
| ATOM | 10016 | CA | THR | B | 522 | 93.586 | 75.164 | 73.595 | 1.00 | 38.96 |
| ATOM | 10017 | C | THR | B | 522 | 93.209 | 73.746 | 74.005 | 1.00 | 36.68 |
| ATOM | 10018 | O | THR | B | 522 | 93.399 | 72.791 | 73.270 | 1.00 | 34.63 |
| ATOM | 10019 | CB | THR | B | 522 | 92.328 | 75.864 | 72.997 | 1.00 | 39.60 |
| ATOM | 10020 | OG1 | THR | B | 522 | 92.560 | 77.277 | 72.831 | 1.00 | 38.31 |
| ATOM | 10021 | CG2 | THR | B | 522 | 91.126 | 75.759 | 73.977 | 1.00 | 38.25 |
| ATOM | 10022 | N | LYS | B | 523 | 92.637 | 73.634 | 75.192 | 1.00 | 34.80 |
| ATOM | 10023 | CA | LYS | B | 523 | 92.145 | 72.359 | 75.667 | 1.00 | 33.28 |
| ATOM | 10024 | C | LYS | B | 523 | 90.781 | 72.060 | 75.086 | 1.00 | 30.81 |
| ATOM | 10025 | O | LYS | B | 523 | 89.830 | 72.859 | 75.224 | 1.00 | 28.33 |
| ATOM | 10026 | CB | LYS | B | 523 | 91.939 | 72.389 | 77.167 | 1.00 | 33.48 |
| ATOM | 10027 | CG | LYS | B | 523 | 93.135 | 72.259 | 78.039 | 1.00 | 37.54 |
| ATOM | 10028 | CD | LYS | B | 523 | 92.604 | 72.123 | 79.487 | 1.00 | 41.59 |
| ATOM | 10029 | CE | LYS | B | 523 | 93.644 | 71.666 | 80.482 | 1.00 | 45.90 |
| ATOM | 10030 | NZ | LYS | B | 523 | 92.930 | 71.329 | 81.773 | 1.00 | 48.02 |
| ATOM | 10031 | N | PHE | B | 524 | 90.641 | 70.889 | 74.494 | 1.00 | 29.69 |
| ATOM | 10032 | CA | PHE | B | 524 | 89.278 | 70.448 | 74.102 | 1.00 | 29.81 |
| ATOM | 10033 | C | PHE | B | 524 | 89.023 | 69.054 | 74.626 | 1.00 | 28.20 |
| ATOM | 10034 | O | PHE | B | 524 | 89.818 | 68.140 | 74.403 | 1.00 | 28.48 |
| ATOM | 10035 | CB | PHE | B | 524 | 89.100 | 70.495 | 72.623 | 1.00 | 29.61 |
| ATOM | 10036 | CG | PHE | B | 524 | 89.034 | 71.877 | 72.082 | 1.00 | 31.07 |
| ATOM | 10037 | CD1 | PHE | B | 524 | 87.851 | 72.583 | 72.101 | 1.00 | 30.34 |
| ATOM | 10038 | CD2 | PHE | B | 524 | 90.146 | 72.466 | 71.536 | 1.00 | 30.96 |
| ATOM | 10039 | CE1 | PHE | B | 524 | 87.806 | 73.832 | 71.632 | 1.00 | 30.24 |
| ATOM | 10040 | CE2 | PHE | B | 524 | 90.087 | 73.723 | 71.051 | 1.00 | 29.57 |
| ATOM | 10041 | CZ | PHE | B | 524 | 88.933 | 74.402 | 71.079 | 1.00 | 28.05 |
| ATOM | 10042 | N | TRP | B | 525 | 87.923 | 68.905 | 75.345 | 1.00 | 26.62 |
| ATOM | 10043 | CA | TRP | B | 525 | 87.602 | 67.652 | 76.004 | 1.00 | 25.72 |
| ATOM | 10044 | C | TRP | B | 525 | 86.908 | 66.623 | 75.126 | 1.00 | 25.25 |
| ATOM | 10045 | O | TRP | B | 525 | 86.174 | 66.974 | 74.195 | 1.00 | 24.05 |
| ATOM | 10046 | CB | TRP | B | 525 | 86.732 | 67.965 | 77.216 | 1.00 | 25.79 |
| ATOM | 10047 | CG | TRP | B | 525 | 87.455 | 68.732 | 78.277 | 1.00 | 27.63 |
| ATOM | 10048 | CD1 | TRP | B | 525 | 87.758 | 70.073 | 78.275 | 1.00 | 27.16 |
| ATOM | 10049 | CD2 | TRP | B | 525 | 88.017 | 68.192 | 79.477 | 1.00 | 29.89 |
| ATOM | 10050 | NE1 | TRP | B | 525 | 88.463 | 70.390 | 79.407 | 1.00 | 31.50 |
| ATOM | 10051 | CE2 | TRP | B | 525 | 88.634 | 69.255 | 80.166 | 1.00 | 30.31 |
| ATOM | 10052 | CE3 | TRP | B | 525 | 88.075 | 66.904 | 80.030 | 1.00 | 30.50 |
| ATOM | 10053 | CZ2 | TRP | B | 525 | 89.270 | 69.086 | 81.386 | 1.00 | 30.73 |
| ATOM | 10054 | CZ3 | TRP | B | 525 | 88.697 | 66.730 | 81.249 | 1.00 | 31.25 |
| ATOM | 10055 | CH2 | TRP | B | 525 | 89.290 | 67.823 | 81.923 | 1.00 | 31.94 |
| ATOM | 10056 | N | TYR | B | 526 | 87.121 | 65.356 | 75.449 | 1.00 | 24.69 |
| ATOM | 10057 | CA | TYR | B | 526 | 86.422 | 64.269 | 74.801 | 1.00 | 25.76 |
| ATOM | 10058 | C | TYR | B | 526 | 86.160 | 63.161 | 75.804 | 1.00 | 25.33 |
| ATOM | 10059 | O | TYR | B | 526 | 86.720 | 63.184 | 76.895 | 1.00 | 25.36 |
| ATOM | 10060 | CB | TYR | B | 526 | 87.260 | 63.675 | 73.664 | 1.00 | 26.43 |
| ATOM | 10061 | CG | TYR | B | 526 | 88.489 | 62.959 | 74.141 | 1.00 | 29.07 |
| ATOM | 10062 | CD1 | TYR | B | 526 | 89.675 | 63.649 | 74.354 | 1.00 | 30.32 |
| ATOM | 10063 | CD2 | TYR | B | 526 | 88.466 | 61.605 | 74.380 | 1.00 | 32.46 |
| ATOM | 10064 | CE1 | TYR | B | 526 | 90.807 | 63.009 | 74.809 | 1.00 | 32.27 |
| ATOM | 10065 | CE2 | TYR | B | 526 | 89.594 | 60.943 | 74.818 | 1.00 | 36.27 |
| ATOM | 10066 | CZ | TYR | B | 526 | 90.772 | 61.657 | 75.029 | 1.00 | 36.33 |
| ATOM | 10067 | OH | TYR | B | 526 | 91.899 | 60.992 | 75.453 | 1.00 | 38.46 |
| ATOM | 10068 | N | GLN | B | 527 | 85.284 | 62.222 | 75.439 | 1.00 | 25.09 |
| ATOM | 10069 | CA | GLN | B | 527 | 85.060 | 60.994 | 76.189 | 1.00 | 25.08 |
| ATOM | 10070 | C | GLN | B | 527 | 85.163 | 59.824 | 75.238 | 1.00 | 26.26 |
| ATOM | 10071 | O | GLN | B | 527 | 84.866 | 59.952 | 74.021 | 1.00 | 26.67 |
| ATOM | 10072 | CB | GLN | B | 527 | 83.670 | 60.932 | 76.904 | 1.00 | 25.67 |
| ATOM | 10073 | CG | GLN | B | 527 | 82.391 | 61.144 | 76.040 | 1.00 | 24.49 |
| ATOM | 10074 | CD | GLN | B | 527 | 81.106 | 60.932 | 76.867 | 1.00 | 26.97 |
| ATOM | 10075 | OE1 | GLN | B | 527 | 80.956 | 61.540 | 77.952 | 1.00 | 24.92 |
| ATOM | 10076 | NE2 | GLN | B | 527 | 80.187 | 60.047 | 76.379 | 1.00 | 24.49 |
| ATOM | 10077 | N | MET | B | 528 | 85.608 | 58.691 | 75.773 | 1.00 | 27.54 |
| ATOM | 10078 | CA | MET | B | 528 | 85.525 | 57.424 | 75.062 | 1.00 | 28.64 |
| ATOM | 10079 | C | MET | B | 528 | 84.841 | 56.371 | 75.930 | 1.00 | 28.90 |
| ATOM | 10080 | O | MET | B | 528 | 85.150 | 56.202 | 77.127 | 1.00 | 28.28 |
| ATOM | 10081 | CB | MET | B | 528 | 86.891 | 56.882 | 74.673 | 1.00 | 28.81 |
| ATOM | 10082 | CG | MET | B | 528 | 87.784 | 57.848 | 73.914 | 1.00 | 31.74 |
| ATOM | 10083 | SD | MET | B | 528 | 89.285 | 57.067 | 73.244 | 1.00 | 29.83 |
| ATOM | 10084 | CE | MET | B | 528 | 89.692 | 58.217 | 72.070 | 1.00 | 29.90 |
| ATOM | 10085 | N | ILE | B | 529 | 83.915 | 55.659 | 75.312 | 1.00 | 29.12 |
| ATOM | 10086 | CA | ILE | B | 529 | 83.330 | 54.488 | 75.899 | 1.00 | 29.94 |
| ATOM | 10087 | C | ILE | B | 529 | 84.141 | 53.290 | 75.398 | 1.00 | 30.98 |
| ATOM | 10088 | O | ILE | B | 529 | 84.114 | 52.986 | 74.198 | 1.00 | 30.01 |
| ATOM | 10089 | CB | ILE | B | 529 | 81.866 | 54.401 | 75.481 | 1.00 | 30.15 |
| ATOM | 10090 | CG1 | ILE | B | 529 | 81.154 | 55.633 | 76.046 | 1.00 | 32.73 |
| ATOM | 10091 | CG2 | ILE | B | 529 | 81.281 | 53.175 | 76.084 | 1.00 | 29.20 |
| ATOM | 10092 | CD1 | ILE | B | 529 | 79.855 | 55.866 | 75.526 | 1.00 | 37.12 |
| ATOM | 10093 | N | LEU | B | 530 | 84.865 | 52.626 | 76.311 | 1.00 | 31.29 |
| ATOM | 10094 | CA | LEU | B | 530 | 85.788 | 51.554 | 75.924 | 1.00 | 31.73 |
| ATOM | 10095 | C | LEU | B | 530 | 85.256 | 50.178 | 76.240 | 1.00 | 31.76 |
| ATOM | 10096 | O | LEU | B | 530 | 84.612 | 49.949 | 77.283 | 1.00 | 32.88 |
| ATOM | 10097 | CB | LEU | B | 530 | 87.140 | 51.727 | 76.613 | 1.00 | 32.22 |
| ATOM | 10098 | CG | LEU | B | 530 | 87.690 | 53.151 | 76.564 | 1.00 | 33.27 |
| ATOM | 10099 | CD1 | LEU | B | 530 | 88.595 | 53.449 | 77.722 | 1.00 | 35.58 |
| ATOM | 10100 | CD2 | LEU | B | 530 | 88.414 | 53.337 | 75.272 | 1.00 | 34.59 |
| ATOM | 10101 | N | PRO | B | 531 | 85.511 | 49.245 | 75.346 | 1.00 | 31.94 |
| ATOM | 10102 | CA | PRO | B | 531 | 85.110 | 47.863 | 75.585 | 1.00 | 32.83 |
| ATOM | 10103 | C | PRO | B | 531 | 85.674 | 47.323 | 76.913 | 1.00 | 33.43 |
| ATOM | 10104 | O | PRO | B | 531 | 86.714 | 47.735 | 77.390 | 1.00 | 31.92 |
| ATOM | 10105 | CB | PRO | B | 531 | 85.738 | 47.102 | 74.417 | 1.00 | 32.20 |
| ATOM | 10106 | CG | PRO | B | 531 | 85.898 | 48.097 | 73.360 | 1.00 | 32.54 |
| ATOM | 10107 | CD | PRO | B | 531 | 86.176 | 49.419 | 74.045 | 1.00 | 32.48 |
| ATOM | 10108 | N | PRO | B | 532 | 85.012 | 46.330 | 77.443 | 1.00 | 35.12 |
| ATOM | 10109 | CA | PRO | B | 532 | 85.471 | 45.710 | 78.677 | 1.00 | 37.14 |
| ATOM | 10110 | C | PRO | B | 532 | 86.816 | 45.052 | 78.373 | 1.00 | 38.82 |
| ATOM | 10111 | O | PRO | B | 532 | 87.105 | 44.764 | 77.204 | 1.00 | 39.18 |
| ATOM | 10112 | CB | PRO | B | 532 | 84.402 | 44.675 | 78.985 | 1.00 | 37.36 |
| ATOM | 10113 | CG | PRO | B | 532 | 83.577 | 44.523 | 77.738 | 1.00 | 36.74 |
| ATOM | 10114 | CD | PRO | B | 532 | 83.878 | 45.636 | 76.816 | 1.00 | 35.14 |
| ATOM | 10115 | N | HIS | B | 533 | 87.640 | 44.862 | 79.396 | 1.00 | 41.03 |
| ATOM | 10116 | CA | HIS | B | 533 | 88.966 | 44.280 | 79.212 | 1.00 | 42.46 |
| ATOM | 10117 | C | HIS | B | 533 | 89.682 | 45.018 | 78.103 | 1.00 | 43.42 |
| ATOM | 10118 | O | HIS | B | 533 | 90.328 | 44.421 | 77.263 | 1.00 | 44.13 |
| ATOM | 10119 | CB | HIS | B | 533 | 88.854 | 42.805 | 78.863 | 1.00 | 42.84 |
| ATOM | 10120 | CG | HIS | B | 533 | 88.023 | 42.033 | 79.831 | 1.00 | 43.24 |
| ATOM | 10121 | ND1 | HIS | B | 533 | 87.031 | 41.164 | 79.437 | 1.00 | 43.68 |
| ATOM | 10122 | CD2 | HIS | B | 533 | 88.016 | 42.023 | 81.184 | 1.00 | 46.76 |
| ATOM | 10123 | CE1 | HIS | B | 533 | 86.454 | 40.639 | 80.505 | 1.00 | 44.62 |
| ATOM | 10124 | NE2 | HIS | B | 533 | 87.028 | 41.150 | 81.578 | 1.00 | 46.14 |
| ATOM | 10125 | N | PHE | B | 534 | 89.531 | 46.326 | 78.083 | 1.00 | 43.97 |
| ATOM | 10126 | CA | PHE | B | 534 | 90.203 | 47.128 | 77.080 | 1.00 | 44.87 |
| ATOM | 10127 | C | PHE | B | 534 | 91.678 | 46.784 | 77.047 | 1.00 | 45.83 |
| ATOM | 10128 | O | PHE | B | 534 | 92.276 | 46.550 | 78.082 | 1.00 | 46.41 |
| ATOM | 10129 | CB | PHE | B | 534 | 90.040 | 48.594 | 77.432 | 1.00 | 44.17 |
| ATOM | 10130 | CG | PHE | B | 534 | 90.647 | 49.507 | 76.454 | 1.00 | 44.16 |
| ATOM | 10131 | CD1 | PHE | B | 534 | 90.300 | 49.438 | 75.124 | 1.00 | 45.18 |
| ATOM | 10132 | CD2 | PHE | B | 534 | 91.558 | 50.466 | 76.863 | 1.00 | 45.36 |
| ATOM | 10133 | CE1 | PHE | B | 534 | 90.844 | 50.306 | 74.200 | 1.00 | 45.22 |
| ATOM | 10134 | CE2 | PHE | B | 534 | 92.107 | 51.337 | 75.958 | 1.00 | 46.95 |
| ATOM | 10135 | CZ | PHE | B | 534 | 91.745 | 51.248 | 74.602 | 1.00 | 46.30 |
| ATOM | 10136 | N | ASP | B | 535 | 92.281 | 46.770 | 75.871 | 1.00 | 46.97 |
| ATOM | 10137 | CA | ASP | B | 535 | 93.705 | 46.471 | 75.775 | 1.00 | 47.32 |
| ATOM | 10138 | C | ASP | B | 535 | 94.317 | 47.342 | 74.721 | 1.00 | 47.18 |
| ATOM | 10139 | O | ASP | B | 535 | 94.052 | 47.138 | 73.557 | 1.00 | 46.48 |
| ATOM | 10140 | CB | ASP | B | 535 | 93.932 | 45.013 | 75.400 | 1.00 | 47.73 |
| ATOM | 10141 | CG | ASP | B | 535 | 95.405 | 44.720 | 75.057 | 1.00 | 49.55 |
| ATOM | 10142 | OD1 | ASP | B | 535 | 96.253 | 45.647 | 75.162 | 1.00 | 50.88 |
| ATOM | 10143 | OD2 | ASP | B | 535 | 95.797 | 43.608 | 74.653 | 1.00 | 49.67 |
| ATOM | 10144 | N | LYS | B | 536 | 95.150 | 48.297 | 75.126 | 1.00 | 47.86 |
| ATOM | 10145 | CA | LYS | B | 536 | 95.718 | 49.278 | 74.195 | 1.00 | 48.50 |
| ATOM | 10146 | C | LYS | B | 536 | 96.760 | 48.780 | 73.184 | 1.00 | 48.52 |
| ATOM | 10147 | O | LYS | B | 536 | 97.236 | 49.576 | 72.363 | 1.00 | 47.71 |
| ATOM | 10148 | CB | LYS | B | 536 | 96.286 | 50.480 | 74.944 | 1.00 | 49.03 |
| ATOM | 10149 | CG | LYS | B | 536 | 97.480 | 50.191 | 75.880 | 1.00 | 52.28 |
| ATOM | 10150 | CD | LYS | B | 536 | 97.644 | 51.365 | 76.881 | 1.00 | 55.15 |
| ATOM | 10151 | CE | LYS | B | 536 | 98.639 | 51.039 | 78.025 | 1.00 | 57.36 |
| ATOM | 10152 | NZ | LYS | B | 536 | 98.718 | 52.151 | 79.040 | 1.00 | 56.81 |
| ATOM | 10153 | N | SER | B | 537 | 97.103 | 47.496 | 73.210 | 1.00 | 48.46 |
| ATOM | 10154 | CA | SER | B | 537 | 98.028 | 46.973 | 72.208 | 1.00 | 49.13 |
| ATOM | 10155 | C | SER | B | 537 | 97.243 | 46.475 | 70.993 | 1.00 | 49.24 |
| ATOM | 10156 | O | SER | B | 537 | 97.824 | 45.991 | 70.009 | 1.00 | 49.23 |
| ATOM | 10157 | CB | SER | B | 537 | 98.896 | 45.860 | 72.785 | 1.00 | 49.28 |
| ATOM | 10158 | OG | SER | B | 537 | 98.097 | 44.746 | 73.152 | 1.00 | 50.50 |
| ATOM | 10159 | N | LYS | B | 538 | 95.916 | 46.590 | 71.063 | 1.00 | 48.47 |
| ATOM | 10160 | CA | LYS | B | 538 | 95.075 | 46.210 | 69.931 | 1.00 | 47.89 |
| ATOM | 10161 | C | LYS | B | 538 | 94.537 | 47.443 | 69.249 | 1.00 | 46.38 |
| ATOM | 10162 | O | LYS | B | 538 | 94.581 | 48.545 | 69.788 | 1.00 | 46.40 |
| ATOM | 10163 | CB | LYS | B | 538 | 93.899 | 45.336 | 70.373 | 1.00 | 48.32 |
| ATOM | 10164 | CG | LYS | B | 538 | 94.264 | 43.869 | 70.583 | 1.00 | 50.04 |
| ATOM | 10165 | CD | LYS | B | 538 | 93.423 | 43.238 | 71.688 | 1.00 | 52.37 |
| ATOM | 10166 | CE | LYS | B | 538 | 94.133 | 42.062 | 72.398 | 1.00 | 53.34 |
| ATOM | 10167 | NZ | LYS | B | 538 | 93.285 | 40.818 | 72.399 | 1.00 | 53.62 |
| ATOM | 10168 | N | LYS | B | 539 | 94.022 | 47.262 | 68.050 | 1.00 | 44.87 |
| ATOM | 10169 | CA | LYS | B | 539 | 93.405 | 48.369 | 67.358 | 1.00 | 43.55 |
| ATOM | 10170 | C | LYS | B | 539 | 91.914 | 48.090 | 67.297 | 1.00 | 40.89 |
| ATOM | 10171 | O | LYS | B | 539 | 91.524 | 47.076 | 66.789 | 1.00 | 40.49 |
| ATOM | 10172 | CB | LYS | B | 539 | 94.033 | 48.530 | 65.973 | 1.00 | 44.47 |
| ATOM | 10173 | CG | LYS | B | 539 | 95.538 | 48.675 | 66.038 | 1.00 | 45.62 |
| ATOM | 10174 | CD | LYS | B | 539 | 96.074 | 49.677 | 65.051 | 1.00 | 47.78 |
| ATOM | 10175 | CE | LYS | B | 539 | 97.484 | 50.141 | 65.445 | 1.00 | 48.95 |
| ATOM | 10176 | NZ | LYS | B | 539 | 97.610 | 51.616 | 65.209 | 1.00 | 50.32 |
| ATOM | 10177 | N | TYR | B | 540 | 91.100 | 48.969 | 67.889 | 1.00 | 38.45 |
| ATOM | 10178 | CA | TYR | B | 540 | 89.641 | 48.855 | 67.858 | 1.00 | 35.25 |
| ATOM | 10179 | C | TYR | B | 540 | 89.085 | 49.795 | 66.805 | 1.00 | 34.02 |
| ATOM | 10180 | O | TYR | B | 540 | 89.690 | 50.825 | 66.525 | 1.00 | 33.35 |
| ATOM | 10181 | CB | TYR | B | 540 | 89.030 | 49.243 | 69.212 | 1.00 | 34.98 |
| ATOM | 10182 | CG | TYR | B | 540 | 89.520 | 48.404 | 70.374 | 1.00 | 32.83 |
| ATOM | 10183 | CD1 | TYR | B | 540 | 90.776 | 48.578 | 70.885 | 1.00 | 33.15 |
| ATOM | 10184 | CD2 | TYR | B | 540 | 88.742 | 47.423 | 70.902 | 1.00 | 32.68 |
| ATOM | 10185 | CE1 | TYR | B | 540 | 91.228 | 47.809 | 71.912 | 1.00 | 34.42 |
| ATOM | 10186 | CE2 | TYR | B | 540 | 89.169 | 46.640 | 71.916 | 1.00 | 33.22 |
| ATOM | 10187 | CZ | TYR | B | 540 | 90.420 | 46.832 | 72.421 | 1.00 | 33.72 |
| ATOM | 10188 | OH | TYR | B | 540 | 90.856 | 46.047 | 73.430 | 1.00 | 32.51 |
| ATOM | 10189 | N | PRO | B | 541 | 87.954 | 49.430 | 66.204 | 1.00 | 32.50 |
| ATOM | 10190 | CA | PRO | B | 541 | 87.208 | 50.343 | 65.337 | 1.00 | 31.98 |
| ATOM | 10191 | C | PRO | B | 541 | 86.599 | 51.474 | 66.176 | 1.00 | 31.61 |
| ATOM | 10192 | O | PRO | B | 541 | 86.225 | 51.267 | 67.314 | 1.00 | 31.17 |
| ATOM | 10193 | CB | PRO | B | 541 | 86.109 | 49.453 | 64.786 | 1.00 | 32.02 |
| ATOM | 10194 | CG | PRO | B | 541 | 85.926 | 48.454 | 65.866 | 1.00 | 32.49 |
| ATOM | 10195 | CD | PRO | B | 541 | 87.305 | 48.111 | 66.277 | 1.00 | 32.29 |
| ATOM | 10196 | N | LEU | B | 542 | 86.537 | 52.671 | 65.619 | 1.00 | 31.70 |
| ATOM | 10197 | CA | LEU | B | 542 | 86.027 | 53.796 | 66.352 | 1.00 | 31.15 |
| ATOM | 10198 | C | LEU | B | 542 | 84.801 | 54.432 | 65.736 | 1.00 | 30.44 |
| ATOM | 10199 | O | LEU | B | 542 | 84.798 | 54.793 | 64.525 | 1.00 | 29.93 |
| ATOM | 10200 | CB | LEU | B | 542 | 87.102 | 54.872 | 66.465 | 1.00 | 32.05 |
| ATOM | 10201 | CG | LEU | B | 542 | 86.691 | 55.940 | 67.489 | 1.00 | 34.95 |
| ATOM | 10202 | CD1 | LEU | B | 542 | 87.860 | 56.396 | 68.295 | 1.00 | 35.53 |
| ATOM | 10203 | CD2 | LEU | B | 542 | 86.030 | 57.086 | 66.798 | 1.00 | 35.86 |
| ATOM | 10204 | N | LEU | B | 543 | 83.784 | 54.608 | 66.586 | 1.00 | 29.46 |
| ATOM | 10205 | CA | LEU | B | 543 | 82.586 | 55.368 | 66.227 | 1.00 | 29.14 |
| ATOM | 10206 | C | LEU | B | 543 | 82.555 | 56.698 | 66.958 | 1.00 | 28.16 |
| ATOM | 10207 | O | LEU | B | 543 | 82.537 | 56.755 | 68.212 | 1.00 | 28.34 |
| ATOM | 10208 | CB | LEU | B | 543 | 81.329 | 54.600 | 66.579 | 1.00 | 29.73 |
| ATOM | 10209 | CG | LEU | B | 543 | 80.002 | 55.227 | 66.202 | 1.00 | 30.86 |
| ATOM | 10210 | CD1 | LEU | B | 543 | 78.876 | 54.568 | 66.950 | 1.00 | 29.26 |
| ATOM | 10211 | CD2 | LEU | B | 543 | 80.047 | 56.664 | 66.530 | 1.00 | 36.44 |
| ATOM | 10212 | N | LEU | B | 544 | 82.536 | 57.769 | 66.190 | 1.00 | 26.68 |
| ATOM | 10213 | CA | LEU | B | 544 | 82.401 | 59.105 | 66.760 | 1.00 | 26.88 |
| ATOM | 10214 | C | LEU | B | 544 | 80.904 | 59.502 | 66.795 | 1.00 | 27.16 |
| ATOM | 10215 | O | LEU | B | 544 | 80.256 | 59.720 | 65.735 | 1.00 | 26.33 |
| ATOM | 10216 | CB | LEU | B | 544 | 83.171 | 60.079 | 65.918 | 1.00 | 26.04 |
| ATOM | 10217 | CG | LEU | B | 544 | 83.254 | 61.524 | 66.311 | 1.00 | 27.83 |
| ATOM | 10218 | CD1 | LEU | B | 544 | 84.240 | 61.777 | 67.414 | 1.00 | 28.08 |
| ATOM | 10219 | CD2 | LEU | B | 544 | 83.663 | 62.290 | 65.115 | 1.00 | 31.47 |
| ATOM | 10220 | N | ASP | B | 545 | 80.377 | 59.541 | 68.019 | 1.00 | 26.72 |
| ATOM | 10221 | CA | ASP | B | 545 | 79.019 | 59.952 | 68.320 | 1.00 | 26.49 |
| ATOM | 10222 | C | ASP | B | 545 | 79.052 | 61.471 | 68.519 | 1.00 | 26.44 |
| ATOM | 10223 | O | ASP | B | 545 | 79.704 | 61.954 | 69.427 | 1.00 | 24.96 |
| ATOM | 10224 | CB | ASP | B | 545 | 78.543 | 59.222 | 69.552 | 1.00 | 26.52 |
| ATOM | 10225 | CG | ASP | B | 545 | 77.240 | 59.745 | 70.081 | 1.00 | 28.25 |
| ATOM | 10226 | OD1 | ASP | B | 545 | 76.638 | 60.688 | 69.494 | 1.00 | 31.33 |
| ATOM | 10227 | OD2 | ASP | B | 545 | 76.751 | 59.265 | 71.091 | 1.00 | 29.13 |
| ATOM | 10228 | N | VAL | B | 546 | 78.403 | 62.200 | 67.611 | 1.00 | 26.32 |
| ATOM | 10229 | CA | VAL | B | 546 | 78.468 | 63.659 | 67.571 | 1.00 | 26.13 |
| ATOM | 10230 | C | VAL | B | 546 | 77.144 | 64.381 | 67.756 | 1.00 | 25.53 |
| ATOM | 10231 | O | VAL | B | 546 | 76.100 | 63.921 | 67.300 | 1.00 | 25.95 |
| ATOM | 10232 | CB | VAL | B | 546 | 79.046 | 64.135 | 66.206 | 1.00 | 26.98 |
| ATOM | 10233 | CG1 | VAL | B | 546 | 78.388 | 63.436 | 65.031 | 1.00 | 29.08 |
| ATOM | 10234 | CG2 | VAL | B | 546 | 78.880 | 65.635 | 66.016 | 1.00 | 27.58 |
| ATOM | 10235 | N | TYR | B | 547 | 77.173 | 65.525 | 68.423 | 1.00 | 25.15 |
| ATOM | 10236 | CA | TYR | B | 547 | 76.002 | 66.388 | 68.456 | 1.00 | 25.44 |
| ATOM | 10237 | C | TYR | B | 547 | 76.560 | 67.668 | 67.953 | 1.00 | 25.50 |
| ATOM | 10238 | O | TYR | B | 547 | 76.371 | 67.991 | 66.794 | 1.00 | 24.39 |
| ATOM | 10239 | CB | TYR | B | 547 | 75.324 | 66.549 | 69.854 | 1.00 | 25.83 |
| ATOM | 10240 | CG | TYR | B | 547 | 74.125 | 67.470 | 69.728 | 1.00 | 26.34 |
| ATOM | 10241 | CD1 | TYR | B | 547 | 72.982 | 67.064 | 68.992 | 1.00 | 26.01 |
| ATOM | 10242 | CD2 | TYR | B | 547 | 74.141 | 68.760 | 70.256 | 1.00 | 26.81 |
| ATOM | 10243 | CE1 | TYR | B | 547 | 71.875 | 67.901 | 68.834 | 1.00 | 23.53 |
| ATOM | 10244 | CE2 | TYR | B | 547 | 73.047 | 69.620 | 70.111 | 1.00 | 23.51 |
| ATOM | 10245 | CZ | TYR | B | 547 | 71.933 | 69.192 | 69.351 | 1.00 | 24.77 |
| ATOM | 10246 | OH | TYR | B | 547 | 70.885 | 70.024 | 69.172 | 1.00 | 19.38 |
| ATOM | 10247 | N | ALA | B | 548 | 77.254 | 68.387 | 68.860 | 1.00 | 25.88 |
| ATOM | 10248 | CA | ALA | B | 548 | 78.052 | 69.568 | 68.561 | 1.00 | 24.96 |
| ATOM | 10249 | C | ALA | B | 548 | 77.318 | 70.841 | 68.194 | 1.00 | 24.67 |
| ATOM | 10250 | O | ALA | B | 548 | 77.898 | 71.723 | 67.623 | 1.00 | 23.45 |
| ATOM | 10251 | CB | ALA | B | 548 | 79.087 | 69.238 | 67.493 | 1.00 | 26.28 |
| ATOM | 10252 | N | GLY | B | 549 | 76.050 | 70.966 | 68.542 | 1.00 | 25.80 |
| ATOM | 10253 | CA | GLY | B | 549 | 75.344 | 72.240 | 68.353 | 1.00 | 25.68 |
| ATOM | 10254 | C | GLY | B | 549 | 75.859 | 73.322 | 69.335 | 1.00 | 26.52 |
| ATOM | 10255 | O | GLY | B | 549 | 76.501 | 73.033 | 70.370 | 1.00 | 24.55 |
| ATOM | 10256 | N | PRO | B | 550 | 75.584 | 74.570 | 69.005 | 1.00 | 27.31 |
| ATOM | 10257 | CA | PRO | B | 550 | 76.015 | 75.694 | 69.819 | 1.00 | 28.20 |
| ATOM | 10258 | C | PRO | B | 550 | 75.621 | 75.464 | 71.271 | 1.00 | 28.35 |
| ATOM | 10259 | O | PRO | B | 550 | 74.472 | 75.140 | 71.498 | 1.00 | 26.88 |
| ATOM | 10260 | CB | PRO | B | 550 | 75.212 | 76.869 | 69.264 | 1.00 | 28.88 |
| ATOM | 10261 | CG | PRO | B | 550 | 74.711 | 76.461 | 67.951 | 1.00 | 29.49 |
| ATOM | 10262 | CD | PRO | B | 550 | 74.844 | 74.988 | 67.808 | 1.00 | 28.76 |
| ATOM | 10263 | N | CYS | B | 551 | 76.579 | 75.576 | 72.190 | 1.00 | 28.41 |
| ATOM | 10264 | CA | CYS | B | 551 | 76.368 | 75.469 | 73.645 | 1.00 | 29.87 |
| ATOM | 10265 | C | CYS | B | 551 | 76.268 | 74.041 | 74.183 | 1.00 | 29.72 |
| ATOM | 10266 | O | CYS | B | 551 | 76.151 | 73.840 | 75.388 | 1.00 | 28.91 |
| ATOM | 10267 | CB | CYS | B | 551 | 75.140 | 76.259 | 74.131 | 1.00 | 30.07 |
| ATOM | 10268 | SG | CYS | B | 551 | 74.882 | 77.910 | 73.466 | 1.00 | 36.88 |
| ATOM | 10269 | N | SER | B | 552 | 76.353 | 73.047 | 73.314 | 1.00 | 29.28 |
| ATOM | 10270 | CA | SER | B | 552 | 76.260 | 71.681 | 73.761 | 1.00 | 29.02 |
| ATOM | 10271 | C | SER | B | 552 | 77.538 | 71.129 | 74.374 | 1.00 | 27.88 |
| ATOM | 10272 | O | SER | B | 552 | 78.649 | 71.647 | 74.221 | 1.00 | 28.06 |
| ATOM | 10273 | CB | SER | B | 552 | 75.885 | 70.784 | 72.583 | 1.00 | 29.55 |
| ATOM | 10274 | OG | SER | B | 552 | 76.966 | 70.759 | 71.674 | 1.00 | 32.67 |
| ATOM | 10275 | N | GLN | B | 553 | 77.347 | 70.024 | 75.047 | 1.00 | 27.04 |
| ATOM | 10276 | CA | GLN | B | 553 | 78.404 | 69.312 | 75.723 | 1.00 | 26.04 |
| ATOM | 10277 | C | GLN | B | 553 | 78.065 | 67.857 | 75.650 | 1.00 | 25.26 |
| ATOM | 10278 | O | GLN | B | 553 | 77.026 | 67.447 | 76.167 | 1.00 | 23.05 |
| ATOM | 10279 | CB | GLN | B | 553 | 78.451 | 69.697 | 77.197 | 1.00 | 25.65 |
| ATOM | 10280 | CG | GLN | B | 553 | 79.552 | 68.978 | 77.973 | 1.00 | 24.70 |
| ATOM | 10281 | CD | GLN | B | 553 | 79.844 | 69.624 | 79.349 | 1.00 | 26.63 |
| ATOM | 10282 | OE1 | GLN | B | 553 | 79.084 | 69.437 | 80.312 | 1.00 | 25.51 |
| ATOM | 10283 | NE2 | GLN | B | 553 | 80.960 | 70.334 | 79.448 | 1.00 | 24.19 |
| ATOM | 10284 | N | LYS | B | 554 | 78.976 | 67.103 | 75.064 | 1.00 | 24.65 |
| ATOM | 10285 | CA | LYS | B | 554 | 78.866 | 65.660 | 74.903 | 1.00 | 25.85 |
| ATOM | 10286 | C | LYS | B | 554 | 79.982 | 64.914 | 75.646 | 1.00 | 25.86 |
| ATOM | 10287 | O | LYS | B | 554 | 80.008 | 63.692 | 75.644 | 1.00 | 24.49 |
| ATOM | 10288 | CB | LYS | B | 554 | 78.995 | 65.306 | 73.420 | 1.00 | 26.26 |
| ATOM | 10289 | CG | LYS | B | 554 | 77.701 | 65.173 | 72.671 | 1.00 | 29.51 |
| ATOM | 10290 | CD | LYS | B | 554 | 77.061 | 63.802 | 72.931 | 1.00 | 32.34 |
| ATOM | 10291 | CE | LYS | B | 554 | 76.806 | 63.009 | 71.600 | 1.00 | 31.09 |
| ATOM | 10292 | NZ | LYS | B | 554 | 75.734 | 61.984 | 71.855 | 1.00 | 29.67 |
| ATOM | 10293 | N | ALA | B | 555 | 80.935 | 65.626 | 76.250 | 1.00 | 25.91 |
| ATOM | 10294 | CA | ALA | B | 555 | 81.943 | 64.935 | 77.043 | 1.00 | 26.38 |
| ATOM | 10295 | C | ALA | B | 555 | 81.626 | 65.285 | 78.461 | 1.00 | 25.75 |
| ATOM | 10296 | O | ALA | B | 555 | 81.709 | 66.428 | 78.843 | 1.00 | 26.47 |
| ATOM | 10297 | CB | ALA | B | 555 | 83.432 | 65.350 | 76.647 | 1.00 | 26.01 |
| ATOM | 10298 | N | ASP | B | 556 | 81.192 | 64.321 | 79.245 | 1.00 | 25.63 |
| ATOM | 10299 | CA | ASP | B | 556 | 80.840 | 64.644 | 80.594 | 1.00 | 25.67 |
| ATOM | 10300 | C | ASP | B | 556 | 81.001 | 63.486 | 81.553 | 1.00 | 25.52 |
| ATOM | 10301 | O | ASP | B | 556 | 81.546 | 62.454 | 81.198 | 1.00 | 24.68 |
| ATOM | 10302 | CB | ASP | B | 556 | 79.452 | 65.282 | 80.678 | 1.00 | 25.54 |
| ATOM | 10303 | CG | ASP | B | 556 | 78.306 | 64.376 | 80.212 | 1.00 | 26.43 |
| ATOM | 10304 | OD1 | ASP | B | 556 | 78.346 | 63.149 | 80.346 | 1.00 | 25.95 |
| ATOM | 10305 | OD2 | ASP | B | 556 | 77.258 | 64.873 | 79.724 | 1.00 | 32.39 |
| ATOM | 10306 | N | THR | B | 557 | 80.553 | 63.677 | 82.780 | 1.00 | 25.33 |
| ATOM | 10307 | CA | THR | B | 557 | 80.737 | 62.642 | 83.791 | 1.00 | 25.75 |
| ATOM | 10308 | C | THR | B | 557 | 79.481 | 61.895 | 84.114 | 1.00 | 25.51 |
| ATOM | 10309 | O | THR | B | 557 | 79.426 | 61.174 | 85.121 | 1.00 | 25.86 |
| ATOM | 10310 | CB | THR | B | 557 | 81.342 | 63.228 | 85.076 | 1.00 | 25.89 |
| ATOM | 10311 | OG1 | THR | B | 557 | 80.558 | 64.341 | 85.553 | 1.00 | 26.22 |
| ATOM | 10312 | CG2 | THR | B | 557 | 82.713 | 63.817 | 84.788 | 1.00 | 25.47 |
| ATOM | 10313 | N | VAL | B | 558 | 78.488 | 61.971 | 83.241 | 1.00 | 25.81 |
| ATOM | 10314 | CA | VAL | B | 558 | 77.274 | 61.221 | 83.502 | 1.00 | 26.28 |
| ATOM | 10315 | C | VAL | B | 558 | 77.351 | 59.740 | 83.115 | 1.00 | 26.36 |
| ATOM | 10316 | O | VAL | B | 558 | 78.028 | 59.339 | 82.181 | 1.00 | 25.31 |
| ATOM | 10317 | CB | VAL | B | 558 | 75.968 | 61.994 | 83.088 | 1.00 | 26.68 |
| ATOM | 10318 | CG1 | VAL | B | 558 | 76.224 | 63.016 | 82.165 | 1.00 | 29.23 |
| ATOM | 10319 | CG2 | VAL | B | 558 | 74.827 | 61.107 | 82.617 | 1.00 | 27.76 |
| ATOM | 10320 | N | PHE | B | 559 | 76.723 | 58.928 | 83.955 | 1.00 | 26.78 |
| ATOM | 10321 | CA | PHE | B | 559 | 76.582 | 57.528 | 83.705 | 1.00 | 27.96 |
| ATOM | 10322 | C | PHE | B | 559 | 75.394 | 57.309 | 82.782 | 1.00 | 28.30 |
| ATOM | 10323 | O | PHE | B | 559 | 74.333 | 57.847 | 83.032 | 1.00 | 27.33 |
| ATOM | 10324 | CB | PHE | B | 559 | 76.247 | 56.831 | 84.973 | 1.00 | 27.82 |
| ATOM | 10325 | CG | PHE | B | 559 | 76.111 | 55.368 | 84.822 | 1.00 | 29.69 |
| ATOM | 10326 | CD1 | PHE | B | 559 | 77.235 | 54.556 | 84.834 | 1.00 | 34.43 |
| ATOM | 10327 | CD2 | PHE | B | 559 | 74.868 | 54.785 | 84.700 | 1.00 | 29.96 |
| ATOM | 10328 | CE1 | PHE | B | 559 | 77.101 | 53.163 | 84.721 | 1.00 | 36.18 |
| ATOM | 10329 | CE2 | PHE | B | 559 | 74.739 | 53.426 | 84.567 | 1.00 | 31.21 |
| ATOM | 10330 | CZ | PHE | B | 559 | 75.848 | 52.609 | 84.584 | 1.00 | 32.62 |
| ATOM | 10331 | N | ARG | B | 560 | 75.570 | 56.505 | 81.744 | 1.00 | 29.23 |
| ATOM | 10332 | CA | ARG | B | 560 | 74.450 | 56.203 | 80.830 | 1.00 | 30.37 |
| ATOM | 10333 | C | ARG | B | 560 | 74.486 | 54.755 | 80.377 | 1.00 | 30.72 |
| ATOM | 10334 | O | ARG | B | 560 | 75.569 | 54.167 | 80.276 | 1.00 | 31.32 |
| ATOM | 10335 | CB | ARG | B | 560 | 74.526 | 57.065 | 79.582 | 1.00 | 30.18 |
| ATOM | 10336 | CG | ARG | B | 560 | 74.389 | 58.556 | 79.805 | 1.00 | 31.05 |
| ATOM | 10337 | CD | ARG | B | 560 | 74.486 | 59.389 | 78.504 | 1.00 | 33.99 |
| ATOM | 10338 | NE | ARG | B | 560 | 74.310 | 60.812 | 78.810 | 1.00 | 34.92 |
| ATOM | 10339 | CZ | ARG | B | 560 | 75.281 | 61.655 | 79.082 | 1.00 | 32.60 |
| ATOM | 10340 | NH1 | ARG | B | 560 | 76.539 | 61.256 | 79.039 | 1.00 | 33.75 |
| ATOM | 10341 | NH2 | ARG | B | 560 | 74.989 | 62.912 | 79.404 | 1.00 | 31.64 |
| ATOM | 10342 | N | LEU | B | 561 | 73.300 | 54.189 | 80.127 | 1.00 | 30.35 |
| ATOM | 10343 | CA | LEU | B | 561 | 73.153 | 52.884 | 79.516 | 1.00 | 29.48 |
| ATOM | 10344 | C | LEU | B | 561 | 72.453 | 53.147 | 78.209 | 1.00 | 28.29 |
| ATOM | 10345 | O | LEU | B | 561 | 71.287 | 53.421 | 78.178 | 1.00 | 27.23 |
| ATOM | 10346 | CB | LEU | B | 561 | 72.367 | 51.939 | 80.391 | 1.00 | 30.20 |
| ATOM | 10347 | CG | LEU | B | 561 | 73.127 | 51.661 | 81.716 | 1.00 | 32.81 |
| ATOM | 10348 | CD1 | LEU | B | 561 | 72.160 | 51.200 | 82.755 | 1.00 | 34.14 |
| ATOM | 10349 | CD2 | LEU | B | 561 | 74.223 | 50.640 | 81.489 | 1.00 | 32.06 |
| ATOM | 10350 | N | ASN | B | 562 | 73.195 | 53.060 | 77.119 | 1.00 | 27.42 |
| ATOM | 10351 | CA | ASN | B | 562 | 72.622 | 53.400 | 75.835 | 1.00 | 27.13 |
| ATOM | 10352 | C | ASN | B | 562 | 73.192 | 52.504 | 74.724 | 1.00 | 26.83 |
| ATOM | 10353 | O | ASN | B | 562 | 73.792 | 51.486 | 75.016 | 1.00 | 25.75 |
| ATOM | 10354 | CB | ASN | B | 562 | 72.836 | 54.891 | 75.558 | 1.00 | 26.53 |
| ATOM | 10355 | CG | ASN | B | 562 | 74.324 | 55.312 | 75.579 | 1.00 | 26.20 |
| ATOM | 10356 | OD1 | ASN | B | 562 | 74.606 | 56.482 | 75.619 | 1.00 | 27.64 |
| ATOM | 10357 | ND2 | ASN | B | 562 | 75.240 | 54.376 | 75.534 | 1.00 | 24.14 |
| ATOM | 10358 | N | TRP | B | 563 | 72.990 | 52.907 | 73.481 | 1.00 | 26.73 |
| ATOM | 10359 | CA | TRP | B | 563 | 73.399 | 52.136 | 72.317 | 1.00 | 27.54 |
| ATOM | 10360 | C | TRP | B | 563 | 74.895 | 51.986 | 72.371 | 1.00 | 27.72 |
| ATOM | 10361 | O | TRP | B | 563 | 75.411 | 50.923 | 72.167 | 1.00 | 27.64 |
| ATOM | 10362 | CB | TRP | B | 563 | 72.950 | 52.860 | 71.028 | 1.00 | 27.25 |
| ATOM | 10363 | CG | TRP | B | 563 | 73.216 | 52.132 | 69.715 | 1.00 | 27.56 |
| ATOM | 10364 | CD1 | TRP | B | 563 | 72.896 | 50.855 | 69.432 | 1.00 | 27.76 |
| ATOM | 10365 | CD2 | TRP | B | 563 | 73.789 | 52.670 | 68.514 | 1.00 | 26.74 |
| ATOM | 10366 | NE1 | TRP | B | 563 | 73.226 | 50.548 | 68.142 | 1.00 | 27.33 |
| ATOM | 10367 | CE2 | TRP | B | 563 | 73.773 | 51.646 | 67.548 | 1.00 | 27.18 |
| ATOM | 10368 | CE3 | TRP | B | 563 | 74.269 | 53.925 | 68.140 | 1.00 | 27.86 |
| ATOM | 10369 | CZ2 | TRP | B | 563 | 74.269 | 51.811 | 66.264 | 1.00 | 27.69 |
| ATOM | 10370 | CZ3 | TRP | B | 563 | 74.787 | 54.087 | 66.874 | 1.00 | 25.86 |
| ATOM | 10371 | CH2 | TRP | B | 563 | 74.769 | 53.042 | 65.941 | 1.00 | 27.84 |
| ATOM | 10372 | N | ALA | B | 564 | 75.586 | 53.081 | 72.667 | 1.00 | 29.15 |
| ATOM | 10373 | CA | ALA | B | 564 | 77.041 | 53.060 | 72.754 | 1.00 | 29.28 |
| ATOM | 10374 | C | ALA | B | 564 | 77.493 | 52.026 | 73.771 | 1.00 | 28.68 |
| ATOM | 10375 | O | ALA | B | 564 | 78.456 | 51.342 | 73.540 | 1.00 | 29.79 |
| ATOM | 10376 | CB | ALA | B | 564 | 77.594 | 54.448 | 73.089 | 1.00 | 28.92 |
| ATOM | 10377 | N | THR | B | 565 | 76.753 | 51.852 | 74.846 | 1.00 | 28.77 |
| ATOM | 10378 | CA | THR | B | 565 | 77.126 | 50.862 | 75.825 | 1.00 | 28.57 |
| ATOM | 10379 | C | THR | B | 565 | 77.145 | 49.506 | 75.150 | 1.00 | 28.84 |
| ATOM | 10380 | O | THR | B | 565 | 77.973 | 48.662 | 75.487 | 1.00 | 28.94 |
| ATOM | 10381 | CB | THR | B | 565 | 76.116 | 50.787 | 76.990 | 1.00 | 28.72 |
| ATOM | 10382 | OG1 | THR | B | 565 | 75.860 | 52.081 | 77.558 | 1.00 | 27.98 |
| ATOM | 10383 | CG2 | THR | B | 565 | 76.650 | 49.863 | 78.157 | 1.00 | 28.57 |
| ATOM | 10384 | N | TYR | B | 566 | 76.151 | 49.247 | 74.309 | 1.00 | 28.41 |
| ATOM | 10385 | CA | TYR | B | 566 | 76.064 | 47.974 | 73.639 | 1.00 | 28.41 |
| ATOM | 10386 | C | TYR | B | 566 | 77.154 | 47.850 | 72.595 | 1.00 | 28.23 |
| ATOM | 10387 | O | TYR | B | 566 | 77.761 | 46.806 | 72.467 | 1.00 | 29.10 |
| ATOM | 10388 | CB | TYR | B | 566 | 74.697 | 47.782 | 72.987 | 1.00 | 29.06 |
| ATOM | 10389 | CG | TYR | B | 566 | 74.773 | 47.080 | 71.676 | 1.00 | 30.04 |
| ATOM | 10390 | CD1 | TYR | B | 566 | 75.035 | 45.721 | 71.619 | 1.00 | 32.31 |
| ATOM | 10391 | CD2 | TYR | B | 566 | 74.620 | 47.779 | 70.481 | 1.00 | 32.97 |
| ATOM | 10392 | CE1 | TYR | B | 566 | 75.113 | 45.058 | 70.415 | 1.00 | 33.88 |
| ATOM | 10393 | CE2 | TYR | B | 566 | 74.684 | 47.110 | 69.249 | 1.00 | 35.94 |
| ATOM | 10394 | CZ | TYR | B | 566 | 74.946 | 45.753 | 69.229 | 1.00 | 34.02 |
| ATOM | 10395 | OH | TYR | B | 566 | 75.052 | 45.081 | 68.042 | 1.00 | 33.67 |
| ATOM | 10396 | N | LEU | B | 567 | 77.455 | 48.913 | 71.878 | 1.00 | 27.02 |
| ATOM | 10397 | CA | LEU | B | 567 | 78.478 | 48.806 | 70.859 | 1.00 | 26.41 |
| ATOM | 10398 | C | LEU | B | 567 | 79.879 | 48.455 | 71.416 | 1.00 | 27.00 |
| ATOM | 10399 | O | LEU | B | 567 | 80.671 | 47.724 | 70.785 | 1.00 | 24.89 |
| ATOM | 10400 | CB | LEU | B | 567 | 78.526 | 50.105 | 70.052 | 1.00 | 25.83 |
| ATOM | 10401 | CG | LEU | B | 567 | 77.258 | 50.402 | 69.240 | 1.00 | 25.67 |
| ATOM | 10402 | CD1 | LEU | B | 567 | 77.333 | 51.828 | 68.696 | 1.00 | 25.99 |
| ATOM | 10403 | CD2 | LEU | B | 567 | 77.090 | 49.382 | 68.094 | 1.00 | 24.78 |
| ATOM | 10404 | N | ALA | B | 568 | 80.202 | 49.026 | 72.571 | 1.00 | 27.42 |
| ATOM | 10405 | CA | ALA | B | 568 | 81.472 | 48.748 | 73.227 | 1.00 | 27.76 |
| ATOM | 10406 | C | ALA | B | 568 | 81.442 | 47.394 | 73.959 | 1.00 | 28.48 |
| ATOM | 10407 | O | ALA | B | 568 | 82.362 | 46.593 | 73.864 | 1.00 | 29.45 |
| ATOM | 10408 | CB | ALA | B | 568 | 81.778 | 49.865 | 74.201 | 1.00 | 28.47 |
| ATOM | 10409 | N | SER | B | 569 | 80.377 | 47.113 | 74.684 | 1.00 | 28.55 |
| ATOM | 10410 | CA | SER | B | 569 | 80.317 | 45.863 | 75.419 | 1.00 | 28.79 |
| ATOM | 10411 | C | SER | B | 569 | 80.205 | 44.601 | 74.539 | 1.00 | 30.00 |
| ATOM | 10412 | O | SER | B | 569 | 80.818 | 43.591 | 74.827 | 1.00 | 28.42 |
| ATOM | 10413 | CB | SER | B | 569 | 79.176 | 45.921 | 76.365 | 1.00 | 28.03 |
| ATOM | 10414 | OG | SER | B | 569 | 78.903 | 44.674 | 76.945 | 1.00 | 30.41 |
| ATOM | 10415 | N | THR | B | 570 | 79.421 | 44.651 | 73.462 | 1.00 | 30.76 |
| ATOM | 10416 | CA | THR | B | 570 | 79.207 | 43.445 | 72.708 | 1.00 | 31.35 |
| ATOM | 10417 | C | THR | B | 570 | 80.023 | 43.441 | 71.426 | 1.00 | 32.03 |
| ATOM | 10418 | O | THR | B | 570 | 80.632 | 42.455 | 71.107 | 1.00 | 32.46 |
| ATOM | 10419 | CB | THR | B | 570 | 77.733 | 43.281 | 72.450 | 1.00 | 32.16 |
| ATOM | 10420 | OG1 | THR | B | 570 | 77.042 | 43.148 | 73.698 | 1.00 | 31.14 |
| ATOM | 10421 | CG2 | THR | B | 570 | 77.408 | 41.967 | 71.676 | 1.00 | 32.40 |
| ATOM | 10422 | N | GLU | B | 571 | 80.065 | 44.565 | 70.725 | 1.00 | 32.39 |
| ATOM | 10423 | CA | GLU | B | 571 | 80.789 | 44.674 | 69.474 | 1.00 | 32.25 |
| ATOM | 10424 | C | GLU | B | 571 | 82.252 | 45.091 | 69.617 | 1.00 | 32.22 |
| ATOM | 10425 | O | GLU | B | 571 | 82.988 | 45.068 | 68.655 | 1.00 | 33.65 |
| ATOM | 10426 | CB | GLU | B | 571 | 80.072 | 45.657 | 68.587 | 1.00 | 32.41 |
| ATOM | 10427 | CG | GLU | B | 571 | 78.621 | 45.281 | 68.383 | 1.00 | 34.67 |
| ATOM | 10428 | CD | GLU | B | 571 | 78.410 | 43.831 | 67.946 | 1.00 | 36.63 |
| ATOM | 10429 | OE1 | GLU | B | 571 | 79.270 | 43.253 | 67.270 | 1.00 | 39.21 |
| ATOM | 10430 | OE2 | GLU | B | 571 | 77.352 | 43.268 | 68.263 | 1.00 | 39.45 |
| ATOM | 10431 | N | ASN | B | 572 | 82.691 | 45.465 | 70.806 | 1.00 | 31.26 |
| ATOM | 10432 | CA | ASN | B | 572 | 84.079 | 45.874 | 70.969 | 1.00 | 31.13 |
| ATOM | 10433 | C | ASN | B | 572 | 84.403 | 47.096 | 70.105 | 1.00 | 29.57 |
| ATOM | 10434 | O | ASN | B | 572 | 85.478 | 47.208 | 69.542 | 1.00 | 29.31 |
| ATOM | 10435 | CB | ASN | B | 572 | 85.074 | 44.688 | 70.746 | 1.00 | 31.11 |
| ATOM | 10436 | CG | ASN | B | 572 | 84.926 | 43.593 | 71.821 | 1.00 | 32.45 |
| ATOM | 10437 | OD1 | ASN | B | 572 | 85.101 | 43.855 | 72.988 | 1.00 | 36.78 |
| ATOM | 10438 | ND2 | ASN | B | 572 | 84.548 | 42.384 | 71.420 | 1.00 | 33.77 |
| ATOM | 10439 | N | ILE | B | 573 | 83.475 | 48.029 | 70.061 | 1.00 | 28.95 |
| ATOM | 10440 | CA | ILE | B | 573 | 83.682 | 49.287 | 69.383 | 1.00 | 29.13 |
| ATOM | 10441 | C | ILE | B | 573 | 83.934 | 50.419 | 70.382 | 1.00 | 28.98 |
| ATOM | 10442 | O | ILE | B | 573 | 83.268 | 50.526 | 71.407 | 1.00 | 28.09 |
| ATOM | 10443 | CB | ILE | B | 573 | 82.455 | 49.633 | 68.576 | 1.00 | 29.67 |
| ATOM | 10444 | CG1 | ILE | B | 573 | 82.180 | 48.531 | 67.555 | 1.00 | 30.91 |
| ATOM | 10445 | CG2 | ILE | B | 573 | 82.679 | 50.955 | 67.858 | 1.00 | 30.51 |
| ATOM | 10446 | CD1 | ILE | B | 573 | 80.871 | 48.702 | 66.885 | 1.00 | 31.18 |
| ATOM | 10447 | N | ILE | B | 574 | 84.877 | 51.286 | 70.056 | 1.00 | 28.35 |
| ATOM | 10448 | CA | ILE | B | 574 | 85.116 | 52.446 | 70.856 | 1.00 | 28.11 |
| ATOM | 10449 | C | ILE | B | 574 | 84.162 | 53.511 | 70.370 | 1.00 | 27.24 |
| ATOM | 10450 | O | ILE | B | 574 | 84.158 | 53.815 | 69.187 | 1.00 | 27.92 |
| ATOM | 10451 | CB | ILE | B | 574 | 86.569 | 52.940 | 70.662 | 1.00 | 28.66 |
| ATOM | 10452 | CG1 | ILE | B | 574 | 87.547 | 52.084 | 71.454 | 1.00 | 29.17 |
| ATOM | 10453 | CG2 | ILE | B | 574 | 86.740 | 54.365 | 71.206 | 1.00 | 30.07 |
| ATOM | 10454 | CD1 | ILE | B | 574 | 88.994 | 52.424 | 71.168 | 1.00 | 29.40 |
| ATOM | 10455 | N | VAL | B | 575 | 83.404 | 54.118 | 71.276 | 1.00 | 26.67 |
| ATOM | 10456 | CA | VAL | B | 575 | 82.464 | 55.191 | 70.921 | 1.00 | 26.59 |
| ATOM | 10457 | C | VAL | B | 575 | 82.890 | 56.485 | 71.557 | 1.00 | 25.86 |
| ATOM | 10458 | O | VAL | B | 575 | 82.791 | 56.645 | 72.765 | 1.00 | 26.51 |
| ATOM | 10459 | CB | VAL | B | 575 | 81.030 | 54.912 | 71.378 | 1.00 | 26.48 |
| ATOM | 10460 | CG1 | VAL | B | 575 | 80.097 | 56.070 | 70.967 | 1.00 | 27.49 |
| ATOM | 10461 | CG2 | VAL | B | 575 | 80.511 | 53.651 | 70.745 | 1.00 | 26.97 |
| ATOM | 10462 | N | ALA | B | 576 | 83.301 | 57.424 | 70.721 | 1.00 | 25.19 |
| ATOM | 10463 | CA | ALA | B | 576 | 83.895 | 58.659 | 71.149 | 1.00 | 24.50 |
| ATOM | 10464 | C | ALA | B | 576 | 83.026 | 59.880 | 70.885 | 1.00 | 24.77 |
| ATOM | 10465 | O | ALA | B | 576 | 82.308 | 59.925 | 69.906 | 1.00 | 24.84 |
| ATOM | 10466 | CB | ALA | B | 576 | 85.165 | 58.819 | 70.433 | 1.00 | 24.61 |
| ATOM | 10467 | N | SER | B | 577 | 83.087 | 60.856 | 71.789 | 1.00 | 24.60 |
| ATOM | 10468 | CA | SER | B | 577 | 82.431 | 62.133 | 71.603 | 1.00 | 24.51 |
| ATOM | 10469 | C | SER | B | 577 | 83.430 | 63.227 | 71.969 | 1.00 | 25.26 |
| ATOM | 10470 | O | SER | B | 577 | 84.197 | 63.074 | 72.918 | 1.00 | 25.83 |
| ATOM | 10471 | CB | SER | B | 577 | 81.194 | 62.194 | 72.457 | 1.00 | 24.10 |
| ATOM | 10472 | OG | SER | B | 577 | 80.266 | 61.204 | 72.035 | 1.00 | 26.22 |
| ATOM | 10473 | N | PHE | B | 578 | 83.395 | 64.326 | 71.232 | 1.00 | 25.14 |
| ATOM | 10474 | CA | PHE | B | 578 | 84.330 | 65.408 | 71.390 | 1.00 | 25.35 |
| ATOM | 10475 | C | PHE | B | 578 | 83.595 | 66.734 | 71.388 | 1.00 | 26.04 |
| ATOM | 10476 | O | PHE | B | 578 | 82.664 | 66.941 | 70.584 | 1.00 | 25.63 |
| ATOM | 10477 | CB | PHE | B | 578 | 85.270 | 65.367 | 70.176 | 1.00 | 26.44 |
| ATOM | 10478 | CG | PHE | B | 578 | 86.254 | 66.500 | 70.104 | 1.00 | 24.81 |
| ATOM | 10479 | CD1 | PHE | B | 578 | 87.358 | 66.525 | 70.906 | 1.00 | 27.02 |
| ATOM | 10480 | CD2 | PHE | B | 578 | 86.091 | 67.491 | 69.201 | 1.00 | 23.55 |
| ATOM | 10481 | CE1 | PHE | B | 578 | 88.302 | 67.543 | 70.779 | 1.00 | 27.15 |
| ATOM | 10482 | CE2 | PHE | B | 578 | 86.994 | 68.524 | 69.112 | 1.00 | 28.20 |
| ATOM | 10483 | CZ | PHE | B | 578 | 88.096 | 68.550 | 69.925 | 1.00 | 24.21 |
| ATOM | 10484 | N | ASP | B | 579 | 84.025 | 67.638 | 72.254 | 1.00 | 24.66 |
| ATOM | 10485 | CA | ASP | B | 579 | 83.457 | 68.981 | 72.325 | 1.00 | 25.26 |
| ATOM | 10486 | C | ASP | B | 579 | 84.456 | 70.023 | 71.763 | 1.00 | 25.12 |
| ATOM | 10487 | O | ASP | B | 579 | 85.415 | 70.421 | 72.460 | 1.00 | 23.61 |
| ATOM | 10488 | CB | ASP | B | 579 | 83.183 | 69.348 | 73.791 | 1.00 | 25.33 |
| ATOM | 10489 | CG | ASP | B | 579 | 82.008 | 68.570 | 74.408 | 1.00 | 28.29 |
| ATOM | 10490 | OD1 | ASP | B | 579 | 81.068 | 68.144 | 73.658 | 1.00 | 25.80 |
| ATOM | 10491 | OD2 | ASP | B | 579 | 81.937 | 68.377 | 75.657 | 1.00 | 30.85 |
| ATOM | 10492 | N | GLY | B | 580 | 84.201 | 70.487 | 70.547 | 1.00 | 24.76 |
| ATOM | 10493 | CA | GLY | B | 580 | 85.038 | 71.465 | 69.894 | 1.00 | 25.40 |
| ATOM | 10494 | C | GLY | B | 580 | 84.510 | 72.874 | 69.988 | 1.00 | 25.60 |
| ATOM | 10495 | O | GLY | B | 580 | 83.721 | 73.216 | 70.883 | 1.00 | 25.56 |
| ATOM | 10496 | N | ARG | B | 581 | 84.946 | 73.698 | 69.040 | 1.00 | 25.93 |
| ATOM | 10497 | CA | ARG | B | 581 | 84.535 | 75.068 | 69.006 | 1.00 | 26.46 |
| ATOM | 10498 | C | ARG | B | 581 | 83.043 | 75.079 | 68.851 | 1.00 | 27.29 |
| ATOM | 10499 | O | ARG | B | 581 | 82.451 | 74.176 | 68.217 | 1.00 | 26.72 |
| ATOM | 10500 | CB | ARG | B | 581 | 85.247 | 75.859 | 67.929 | 1.00 | 27.05 |
| ATOM | 10501 | CG | ARG | B | 581 | 86.670 | 76.261 | 68.345 | 1.00 | 28.13 |
| ATOM | 10502 | CD | ARG | B | 581 | 87.510 | 76.860 | 67.244 | 1.00 | 29.07 |
| ATOM | 10503 | NE | ARG | B | 581 | 87.882 | 75.882 | 66.232 | 1.00 | 30.12 |
| ATOM | 10504 | CZ | ARG | B | 581 | 88.502 | 76.185 | 65.083 | 1.00 | 32.47 |
| ATOM | 10505 | NH1 | ARG | B | 581 | 88.795 | 77.444 | 64.794 | 1.00 | 32.23 |
| ATOM | 10506 | NH2 | ARG | B | 581 | 88.793 | 75.237 | 64.205 | 1.00 | 30.51 |
| ATOM | 10507 | N | GLY | B | 582 | 82.436 | 76.075 | 69.504 | 1.00 | 28.33 |
| ATOM | 10508 | CA | GLY | B | 582 | 80.975 | 76.200 | 69.574 | 1.00 | 28.60 |
| ATOM | 10509 | C | GLY | B | 582 | 80.391 | 75.512 | 70.805 | 1.00 | 28.42 |
| ATOM | 10510 | O | GLY | B | 582 | 79.274 | 75.796 | 71.217 | 1.00 | 27.45 |
| ATOM | 10511 | N | SER | B | 583 | 81.152 | 74.603 | 71.414 | 1.00 | 28.74 |
| ATOM | 10512 | CA | SER | B | 583 | 80.652 | 73.903 | 72.605 | 1.00 | 28.30 |
| ATOM | 10513 | C | SER | B | 583 | 80.541 | 74.783 | 73.843 | 1.00 | 27.52 |
| ATOM | 10514 | O | SER | B | 583 | 81.150 | 75.837 | 73.941 | 1.00 | 27.35 |
| ATOM | 10515 | CB | SER | B | 583 | 81.458 | 72.638 | 72.882 | 1.00 | 29.15 |
| ATOM | 10516 | OG | SER | B | 583 | 82.784 | 72.889 | 73.323 | 1.00 | 30.66 |
| ATOM | 10517 | N | GLY | B | 584 | 79.722 | 74.363 | 74.793 | 1.00 | 26.43 |
| ATOM | 10518 | CA | GLY | B | 584 | 79.439 | 75.220 | 75.907 | 1.00 | 26.10 |
| ATOM | 10519 | C | GLY | B | 584 | 80.212 | 74.976 | 77.182 | 1.00 | 25.98 |
| ATOM | 10520 | O | GLY | B | 584 | 80.966 | 74.014 | 77.342 | 1.00 | 24.58 |
| ATOM | 10521 | N | TYR | B | 585 | 79.978 | 75.901 | 78.092 | 1.00 | 26.84 |
| ATOM | 10522 | CA | TYR | B | 585 | 80.396 | 75.781 | 79.483 | 1.00 | 27.30 |
| ATOM | 10523 | C | TYR | B | 585 | 81.883 | 76.015 | 79.734 | 1.00 | 27.23 |
| ATOM | 10524 | O | TYR | B | 585 | 82.332 | 75.746 | 80.831 | 1.00 | 27.02 |
| ATOM | 10525 | CB | TYR | B | 585 | 80.008 | 74.404 | 80.017 | 1.00 | 27.04 |
| ATOM | 10526 | CG | TYR | B | 585 | 78.559 | 74.131 | 79.899 | 1.00 | 28.27 |
| ATOM | 10527 | CD1 | TYR | B | 585 | 77.640 | 74.849 | 80.635 | 1.00 | 30.07 |
| ATOM | 10528 | CD2 | TYR | B | 585 | 78.092 | 73.160 | 79.025 | 1.00 | 31.05 |
| ATOM | 10529 | CE1 | TYR | B | 585 | 76.279 | 74.619 | 80.503 | 1.00 | 30.51 |
| ATOM | 10530 | CE2 | TYR | B | 585 | 76.751 | 72.912 | 78.891 | 1.00 | 31.04 |
| ATOM | 10531 | CZ | TYR | B | 585 | 75.850 | 73.637 | 79.628 | 1.00 | 31.77 |
| ATOM | 10532 | OH | TYR | B | 585 | 74.518 | 73.367 | 79.484 | 1.00 | 32.27 |
| ATOM | 10533 | N | GLN | B | 586 | 82.596 | 76.520 | 78.733 | 1.00 | 27.28 |
| ATOM | 10534 | CA | GLN | B | 586 | 84.018 | 76.789 | 78.792 | 1.00 | 27.85 |
| ATOM | 10535 | C | GLN | B | 586 | 84.362 | 78.225 | 78.330 | 1.00 | 27.62 |
| ATOM | 10536 | O | GLN | B | 586 | 85.498 | 78.510 | 77.913 | 1.00 | 28.03 |
| ATOM | 10537 | CB | GLN | B | 586 | 84.745 | 75.804 | 77.896 | 1.00 | 28.25 |
| ATOM | 10538 | CG | GLN | B | 586 | 84.307 | 74.361 | 78.064 | 1.00 | 29.26 |
| ATOM | 10539 | CD | GLN | B | 586 | 84.428 | 73.587 | 76.784 | 1.00 | 29.61 |
| ATOM | 10540 | OE1 | GLN | B | 586 | 83.382 | 73.308 | 76.103 | 1.00 | 31.20 |
| ATOM | 10541 | NE2 | GLN | B | 586 | 85.680 | 73.306 | 76.374 | 1.00 | 23.71 |
| ATOM | 10542 | N | GLY | B | 587 | 83.372 | 79.107 | 78.359 | 1.00 | 27.18 |
| ATOM | 10543 | CA | GLY | B | 587 | 83.549 | 80.498 | 78.004 | 1.00 | 27.53 |
| ATOM | 10544 | C | GLY | B | 587 | 83.131 | 80.788 | 76.585 | 1.00 | 28.64 |
| ATOM | 10545 | O | GLY | B | 587 | 82.967 | 79.858 | 75.753 | 1.00 | 28.95 |
| ATOM | 10546 | N | ASP | B | 588 | 82.951 | 82.075 | 76.315 | 1.00 | 29.40 |
| ATOM | 10547 | CA | ASP | B | 588 | 82.531 | 82.573 | 75.033 | 1.00 | 31.43 |
| ATOM | 10548 | C | ASP | B | 588 | 83.551 | 82.483 | 73.880 | 1.00 | 32.96 |
| ATOM | 10549 | O | ASP | B | 588 | 83.162 | 82.556 | 72.707 | 1.00 | 33.17 |
| ATOM | 10550 | CB | ASP | B | 588 | 82.111 | 84.024 | 75.157 | 1.00 | 31.39 |
| ATOM | 10551 | CG | ASP | B | 588 | 80.825 | 84.191 | 75.914 | 1.00 | 34.45 |
| ATOM | 10552 | OD1 | ASP | B | 588 | 80.179 | 83.155 | 76.201 | 1.00 | 37.51 |
| ATOM | 10553 | OD2 | ASP | B | 588 | 80.378 | 85.323 | 76.260 | 1.00 | 34.75 |
| ATOM | 10554 | N | LYS | B | 589 | 84.837 | 82.397 | 74.185 | 1.00 | 33.13 |
| ATOM | 10555 | CA | LYS | B | 589 | 85.824 | 82.333 | 73.125 | 1.00 | 33.91 |
| ATOM | 10556 | C | LYS | B | 589 | 85.618 | 81.028 | 72.373 | 1.00 | 32.65 |
| ATOM | 10557 | O | LYS | B | 589 | 85.767 | 80.960 | 71.179 | 1.00 | 33.07 |
| ATOM | 10558 | CB | LYS | B | 589 | 87.264 | 82.380 | 73.678 | 1.00 | 34.26 |
| ATOM | 10559 | CG | LYS | B | 589 | 88.320 | 82.387 | 72.572 | 1.00 | 37.91 |
| ATOM | 10560 | CD | LYS | B | 589 | 89.765 | 82.367 | 73.123 | 1.00 | 42.91 |
| ATOM | 10561 | CE | LYS | B | 589 | 90.868 | 82.113 | 72.032 | 1.00 | 43.62 |
| ATOM | 10562 | NZ | LYS | B | 589 | 92.247 | 81.993 | 72.731 | 1.00 | 43.08 |
| ATOM | 10563 | N | ILE | B | 590 | 85.295 | 79.980 | 73.105 | 1.00 | 31.38 |
| ATOM | 10564 | CA | ILE | B | 590 | 85.024 | 78.707 | 72.500 | 1.00 | 30.12 |
| ATOM | 10565 | C | ILE | B | 590 | 83.590 | 78.656 | 72.024 | 1.00 | 30.27 |
| ATOM | 10566 | O | ILE | B | 590 | 83.325 | 78.310 | 70.873 | 1.00 | 30.58 |
| ATOM | 10567 | CB | ILE | B | 590 | 85.322 | 77.594 | 73.491 | 1.00 | 30.61 |
| ATOM | 10568 | CG1 | ILE | B | 590 | 86.870 | 77.484 | 73.670 | 1.00 | 28.29 |
| ATOM | 10569 | CG2 | ILE | B | 590 | 84.620 | 76.270 | 73.078 | 1.00 | 26.65 |
| ATOM | 10570 | CD1 | ILE | B | 590 | 87.277 | 76.467 | 74.700 | 1.00 | 29.97 |
| ATOM | 10571 | N | MET | B | 591 | 82.651 | 79.022 | 72.871 | 1.00 | 30.17 |
| ATOM | 10572 | CA | MET | B | 591 | 81.267 | 78.887 | 72.482 | 1.00 | 29.62 |
| ATOM | 10573 | C | MET | B | 591 | 80.884 | 79.781 | 71.303 | 1.00 | 29.25 |
| ATOM | 10574 | O | MET | B | 591 | 80.259 | 79.294 | 70.363 | 1.00 | 29.57 |
| ATOM | 10575 | CB | MET | B | 591 | 80.347 | 79.154 | 73.658 | 1.00 | 30.52 |
| ATOM | 10576 | CG | MET | B | 591 | 78.871 | 79.080 | 73.278 | 1.00 | 30.68 |
| ATOM | 10577 | SD | MET | B | 591 | 77.811 | 78.961 | 74.661 | 1.00 | 31.62 |
| ATOM | 10578 | CE | MET | B | 591 | 77.284 | 80.640 | 74.802 | 1.00 | 28.20 |
| ATOM | 10579 | N | HIS | B | 592 | 81.234 | 81.067 | 71.318 | 1.00 | 27.94 |
| ATOM | 10580 | CA | HIS | B | 592 | 80.885 | 81.943 | 70.186 | 1.00 | 27.62 |
| ATOM | 10581 | C | HIS | B | 592 | 81.793 | 81.813 | 68.949 | 1.00 | 27.34 |
| ATOM | 10582 | O | HIS | B | 592 | 81.648 | 82.537 | 67.996 | 1.00 | 27.16 |
| ATOM | 10583 | CB | HIS | B | 592 | 80.884 | 83.419 | 70.609 | 1.00 | 28.46 |
| ATOM | 10584 | CG | HIS | B | 592 | 79.782 | 83.789 | 71.569 | 1.00 | 28.43 |
| ATOM | 10585 | ND1 | HIS | B | 592 | 79.903 | 84.823 | 72.470 | 1.00 | 25.69 |
| ATOM | 10586 | CD2 | HIS | B | 592 | 78.552 | 83.249 | 71.785 | 1.00 | 30.21 |
| ATOM | 10587 | CE1 | HIS | B | 592 | 78.813 | 84.905 | 73.208 | 1.00 | 25.59 |
| ATOM | 10588 | NE2 | HIS | B | 592 | 77.958 | 83.981 | 72.795 | 1.00 | 29.95 |
| ATOM | 10589 | N | ALA | B | 593 | 82.719 | 80.879 | 68.909 | 1.00 | 27.75 |
| ATOM | 10590 | CA | ALA | B | 593 | 83.619 | 80.888 | 67.772 | 1.00 | 28.43 |
| ATOM | 10591 | C | ALA | B | 593 | 82.826 | 80.686 | 66.509 | 1.00 | 29.66 |
| ATOM | 10592 | O | ALA | B | 593 | 83.285 | 80.971 | 65.415 | 1.00 | 29.97 |
| ATOM | 10593 | CB | ALA | B | 593 | 84.587 | 79.805 | 67.910 | 1.00 | 27.97 |
| ATOM | 10594 | N | ILE | B | 594 | 81.618 | 80.172 | 66.664 | 1.00 | 29.79 |
| ATOM | 10595 | CA | ILE | B | 594 | 80.890 | 79.719 | 65.529 | 1.00 | 30.72 |
| ATOM | 10596 | C | ILE | B | 594 | 79.752 | 80.625 | 65.167 | 1.00 | 31.16 |
| ATOM | 10597 | O | ILE | B | 594 | 78.905 | 80.306 | 64.323 | 1.00 | 31.43 |
| ATOM | 10598 | CB | ILE | B | 594 | 80.520 | 78.290 | 65.877 | 1.00 | 31.20 |
| ATOM | 10599 | CG1 | ILE | B | 594 | 80.946 | 77.391 | 64.760 | 1.00 | 31.99 |
| ATOM | 10600 | CG2 | ILE | B | 594 | 79.091 | 78.126 | 66.365 | 1.00 | 31.20 |
| ATOM | 10601 | CD1 | ILE | B | 594 | 81.811 | 76.349 | 65.216 | 1.00 | 33.13 |
| ATOM | 10602 | N | ASN | B | 595 | 79.755 | 81.785 | 65.808 | 1.00 | 32.03 |
| ATOM | 10603 | CA | ASN | B | 595 | 78.712 | 82.778 | 65.639 | 1.00 | 31.64 |
| ATOM | 10604 | C | ASN | B | 595 | 78.553 | 83.096 | 64.183 | 1.00 | 32.87 |
| ATOM | 10605 | O | ASN | B | 595 | 79.546 | 83.274 | 63.459 | 1.00 | 33.90 |
| ATOM | 10606 | CB | ASN | B | 595 | 79.097 | 84.025 | 66.391 | 1.00 | 31.87 |
| ATOM | 10607 | CG | ASN | B | 595 | 78.022 | 85.083 | 66.362 | 1.00 | 31.16 |
| ATOM | 10608 | OD1 | ASN | B | 595 | 76.837 | 84.789 | 66.322 | 1.00 | 32.97 |
| ATOM | 10609 | ND2 | ASN | B | 595 | 78.436 | 86.321 | 66.395 | 1.00 | 30.64 |
| ATOM | 10610 | N | ARG | B | 596 | 77.313 | 83.175 | 63.731 | 1.00 | 32.50 |
| ATOM | 10611 | CA | ARG | B | 596 | 77.044 | 83.449 | 62.327 | 1.00 | 33.09 |
| ATOM | 10612 | C | ARG | B | 596 | 77.798 | 82.494 | 61.422 | 1.00 | 32.46 |
| ATOM | 10613 | O | ARG | B | 596 | 77.969 | 82.765 | 60.243 | 1.00 | 30.88 |
| ATOM | 10614 | CB | ARG | B | 596 | 77.383 | 84.910 | 61.971 | 1.00 | 33.50 |
| ATOM | 10615 | CG | ARG | B | 596 | 76.572 | 85.990 | 62.745 | 1.00 | 36.26 |
| ATOM | 10616 | CD | ARG | B | 596 | 76.985 | 87.482 | 62.409 | 1.00 | 40.45 |
| ATOM | 10617 | NE | ARG | B | 596 | 76.324 | 88.059 | 61.201 | 1.00 | 44.20 |
| ATOM | 10618 | CZ | ARG | B | 596 | 76.860 | 88.114 | 59.978 | 1.00 | 46.01 |
| ATOM | 10619 | NH1 | ARG | B | 596 | 78.084 | 87.620 | 59.739 | 1.00 | 47.70 |
| ATOM | 10620 | NH2 | ARG | B | 596 | 76.170 | 88.672 | 58.989 | 1.00 | 43.53 |
| ATOM | 10621 | N | ARG | B | 597 | 78.256 | 81.368 | 61.943 | 1.00 | 32.93 |
| ATOM | 10622 | CA | ARG | B | 597 | 79.043 | 80.476 | 61.081 | 1.00 | 34.19 |
| ATOM | 10623 | C | ARG | B | 597 | 78.829 | 78.990 | 61.332 | 1.00 | 32.69 |
| ATOM | 10624 | O | ARG | B | 597 | 79.796 | 78.272 | 61.458 | 1.00 | 32.70 |
| ATOM | 10625 | CB | ARG | B | 597 | 80.549 | 80.815 | 61.232 | 1.00 | 36.10 |
| ATOM | 10626 | CG | ARG | B | 597 | 80.956 | 82.200 | 60.699 | 1.00 | 41.65 |
| ATOM | 10627 | CD | ARG | B | 597 | 82.277 | 82.747 | 61.266 | 1.00 | 51.98 |
| ATOM | 10628 | NE | ARG | B | 597 | 82.324 | 82.880 | 62.755 | 1.00 | 58.21 |
| ATOM | 10629 | CZ | ARG | B | 597 | 82.566 | 84.023 | 63.421 | 1.00 | 61.33 |
| ATOM | 10630 | NH1 | ARG | B | 597 | 82.767 | 85.173 | 62.766 | 1.00 | 63.35 |
| ATOM | 10631 | NH2 | ARG | B | 597 | 82.613 | 84.014 | 64.750 | 1.00 | 62.56 |
| ATOM | 10632 | N | LEU | B | 598 | 77.575 | 78.525 | 61.381 | 1.00 | 31.58 |
| ATOM | 10633 | CA | LEU | B | 598 | 77.279 | 77.124 | 61.645 | 1.00 | 31.01 |
| ATOM | 10634 | C | LEU | B | 598 | 77.799 | 76.267 | 60.555 | 1.00 | 30.47 |
| ATOM | 10635 | O | LEU | B | 598 | 77.947 | 76.718 | 59.469 | 1.00 | 31.95 |
| ATOM | 10636 | CB | LEU | B | 598 | 75.768 | 76.869 | 61.795 | 1.00 | 31.31 |
| ATOM | 10637 | CG | LEU | B | 598 | 75.164 | 77.669 | 62.926 | 1.00 | 30.57 |
| ATOM | 10638 | CD1 | LEU | B | 598 | 73.698 | 77.411 | 62.997 | 1.00 | 32.93 |
| ATOM | 10639 | CD2 | LEU | B | 598 | 75.858 | 77.326 | 64.254 | 1.00 | 28.42 |
| ATOM | 10640 | N | GLY | B | 599 | 78.101 | 75.015 | 60.848 | 1.00 | 30.02 |
| ATOM | 10641 | CA | GLY | B | 599 | 78.646 | 74.140 | 59.832 | 1.00 | 29.74 |
| ATOM | 10642 | C | GLY | B | 599 | 80.079 | 74.472 | 59.434 | 1.00 | 29.84 |
| ATOM | 10643 | O | GLY | B | 599 | 80.514 | 74.163 | 58.348 | 1.00 | 29.03 |
| ATOM | 10644 | N | THR | B | 600 | 80.844 | 75.032 | 60.348 | 1.00 | 29.63 |
| ATOM | 10645 | CA | THR | B | 600 | 82.196 | 75.444 | 60.022 | 1.00 | 30.03 |
| ATOM | 10646 | C | THR | B | 600 | 83.197 | 74.870 | 61.038 | 1.00 | 29.73 |
| ATOM | 10647 | O | THR | B | 600 | 83.554 | 73.691 | 60.949 | 1.00 | 29.64 |
| ATOM | 10648 | CB | THR | B | 600 | 82.100 | 76.984 | 59.909 | 1.00 | 31.08 |
| ATOM | 10649 | OG1 | THR | B | 600 | 82.558 | 77.447 | 58.634 | 1.00 | 34.89 |
| ATOM | 10650 | CG2 | THR | B | 600 | 82.794 | 77.710 | 60.930 | 1.00 | 28.28 |
| ATOM | 10651 | N | PHE | B | 601 | 83.606 | 75.627 | 62.044 | 1.00 | 29.95 |
| ATOM | 10652 | CA | PHE | B | 601 | 84.644 | 75.146 | 62.961 | 1.00 | 30.35 |
| ATOM | 10653 | C | PHE | B | 601 | 84.194 | 73.919 | 63.736 | 1.00 | 29.59 |
| ATOM | 10654 | O | PHE | B | 601 | 84.991 | 73.038 | 63.982 | 1.00 | 28.80 |
| ATOM | 10655 | CB | PHE | B | 601 | 85.083 | 76.239 | 63.944 | 1.00 | 30.77 |
| ATOM | 10656 | CG | PHE | B | 601 | 85.644 | 77.480 | 63.290 | 1.00 | 34.23 |
| ATOM | 10657 | CD1 | PHE | B | 601 | 86.546 | 77.401 | 62.245 | 1.00 | 38.59 |
| ATOM | 10658 | CD2 | PHE | B | 601 | 85.292 | 78.735 | 63.746 | 1.00 | 38.13 |
| ATOM | 10659 | CE1 | PHE | B | 601 | 87.070 | 78.560 | 61.663 | 1.00 | 38.92 |
| ATOM | 10660 | CE2 | PHE | B | 601 | 85.821 | 79.893 | 63.162 | 1.00 | 38.48 |
| ATOM | 10661 | CZ | PHE | B | 601 | 86.700 | 79.800 | 62.132 | 1.00 | 38.17 |
| ATOM | 10662 | N | GLU | B | 602 | 82.906 | 73.835 | 64.086 | 1.00 | 29.35 |
| ATOM | 10663 | CA | GLU | B | 602 | 82.423 | 72.673 | 64.819 | 1.00 | 28.58 |
| ATOM | 10664 | C | GLU | B | 602 | 82.525 | 71.384 | 63.979 | 1.00 | 28.39 |
| ATOM | 10665 | O | GLU | B | 602 | 82.773 | 70.290 | 64.510 | 1.00 | 28.66 |
| ATOM | 10666 | CB | GLU | B | 602 | 81.000 | 72.916 | 65.356 | 1.00 | 28.78 |
| ATOM | 10667 | CG | GLU | B | 602 | 79.859 | 72.581 | 64.437 | 1.00 | 27.88 |
| ATOM | 10668 | CD | GLU | B | 602 | 79.514 | 73.699 | 63.503 | 1.00 | 28.39 |
| ATOM | 10669 | OE1 | GLU | B | 602 | 80.433 | 74.414 | 63.025 | 1.00 | 28.79 |
| ATOM | 10670 | OE2 | GLU | B | 602 | 78.311 | 73.818 | 63.212 | 1.00 | 29.14 |
| ATOM | 10671 | N | VAL | B | 603 | 82.423 | 71.528 | 62.663 | 1.00 | 28.03 |
| ATOM | 10672 | CA | VAL | B | 603 | 82.535 | 70.393 | 61.736 | 1.00 | 27.82 |
| ATOM | 10673 | C | VAL | B | 603 | 84.012 | 70.018 | 61.601 | 1.00 | 28.52 |
| ATOM | 10674 | O | VAL | B | 603 | 84.410 | 68.848 | 61.662 | 1.00 | 27.76 |
| ATOM | 10675 | CB | VAL | B | 603 | 81.969 | 70.803 | 60.340 | 1.00 | 27.10 |
| ATOM | 10676 | CG1 | VAL | B | 603 | 82.140 | 69.735 | 59.338 | 1.00 | 27.69 |
| ATOM | 10677 | CG2 | VAL | B | 603 | 80.530 | 71.170 | 60.453 | 1.00 | 28.49 |
| ATOM | 10678 | N | GLU | B | 604 | 84.818 | 71.036 | 61.361 | 1.00 | 29.64 |
| ATOM | 10679 | CA | GLU | B | 604 | 86.261 | 70.877 | 61.261 | 1.00 | 30.17 |
| ATOM | 10680 | C | GLU | B | 604 | 86.855 | 70.214 | 62.486 | 1.00 | 29.17 |
| ATOM | 10681 | O | GLU | B | 604 | 87.694 | 69.330 | 62.376 | 1.00 | 26.66 |
| ATOM | 10682 | CB | GLU | B | 604 | 86.895 | 72.253 | 61.125 | 1.00 | 31.80 |
| ATOM | 10683 | CG | GLU | B | 604 | 86.525 | 72.986 | 59.830 | 1.00 | 35.77 |
| ATOM | 10684 | CD | GLU | B | 604 | 87.043 | 74.421 | 59.795 | 1.00 | 42.21 |
| ATOM | 10685 | OE1 | GLU | B | 604 | 88.206 | 74.647 | 60.303 | 1.00 | 42.15 |
| ATOM | 10686 | OE2 | GLU | B | 604 | 86.277 | 75.314 | 59.270 | 1.00 | 43.50 |
| ATOM | 10687 | N | ASP | B | 605 | 86.378 | 70.625 | 63.661 | 1.00 | 28.35 |
| ATOM | 10688 | CA | ASP | B | 605 | 86.962 | 70.164 | 64.900 | 1.00 | 28.64 |
| ATOM | 10689 | C | ASP | B | 605 | 86.642 | 68.688 | 65.133 | 1.00 | 27.94 |
| ATOM | 10690 | O | ASP | B | 605 | 87.458 | 67.954 | 65.662 | 1.00 | 27.49 |
| ATOM | 10691 | CB | ASP | B | 605 | 86.496 | 71.020 | 66.084 | 1.00 | 29.20 |
| ATOM | 10692 | CG | ASP | B | 605 | 87.165 | 72.419 | 66.138 | 1.00 | 31.44 |
| ATOM | 10693 | OD1 | ASP | B | 605 | 87.929 | 72.797 | 65.213 | 1.00 | 29.89 |
| ATOM | 10694 | OD2 | ASP | B | 605 | 86.931 | 73.228 | 67.085 | 1.00 | 30.59 |
| ATOM | 10695 | N | GLN | B | 606 | 85.462 | 68.242 | 64.730 | 1.00 | 27.69 |
| ATOM | 10696 | CA | GLN | B | 606 | 85.127 | 66.838 | 64.861 | 1.00 | 26.79 |
| ATOM | 10697 | C | GLN | B | 606 | 86.104 | 66.041 | 63.985 | 1.00 | 27.77 |
| ATOM | 10698 | O | GLN | B | 606 | 86.541 | 64.963 | 64.357 | 1.00 | 27.24 |
| ATOM | 10699 | CB | GLN | B | 606 | 83.670 | 66.574 | 64.448 | 1.00 | 26.75 |
| ATOM | 10700 | CG | GLN | B | 606 | 82.590 | 67.157 | 65.402 | 1.00 | 25.51 |
| ATOM | 10701 | CD | GLN | B | 606 | 82.546 | 66.412 | 66.737 | 1.00 | 25.37 |
| ATOM | 10702 | OE1 | GLN | B | 606 | 82.500 | 65.177 | 66.759 | 1.00 | 31.48 |
| ATOM | 10703 | NE2 | GLN | B | 606 | 82.634 | 67.141 | 67.833 | 1.00 | 22.23 |
| ATOM | 10704 | N | ILE | B | 607 | 86.438 | 66.573 | 62.819 | 1.00 | 28.71 |
| ATOM | 10705 | CA | ILE | B | 607 | 87.398 | 65.911 | 61.918 | 1.00 | 30.05 |
| ATOM | 10706 | C | ILE | B | 607 | 88.787 | 65.823 | 62.484 | 1.00 | 30.76 |
| ATOM | 10707 | O | ILE | B | 607 | 89.416 | 64.772 | 62.428 | 1.00 | 31.62 |
| ATOM | 10708 | CB | ILE | B | 607 | 87.475 | 66.626 | 60.579 | 1.00 | 29.64 |
| ATOM | 10709 | CG1 | ILE | B | 607 | 86.180 | 66.412 | 59.831 | 1.00 | 29.14 |
| ATOM | 10710 | CG2 | ILE | B | 607 | 88.632 | 66.044 | 59.715 | 1.00 | 31.57 |
| ATOM | 10711 | CD1 | ILE | B | 607 | 86.001 | 67.251 | 58.585 | 1.00 | 31.21 |
| ATOM | 10712 | N | GLU | B | 608 | 89.277 | 66.925 | 63.029 | 1.00 | 31.23 |
| ATOM | 10713 | CA | GLU | B | 608 | 90.602 | 66.916 | 63.642 | 1.00 | 31.09 |
| ATOM | 10714 | C | GLU | B | 608 | 90.653 | 65.920 | 64.840 | 1.00 | 30.08 |
| ATOM | 10715 | O | GLU | B | 608 | 91.575 | 65.100 | 64.973 | 1.00 | 29.15 |
| ATOM | 10716 | CB | GLU | B | 608 | 90.978 | 68.347 | 63.985 | 1.00 | 31.42 |
| ATOM | 10717 | CG | GLU | B | 608 | 92.234 | 68.546 | 64.831 | 1.00 | 36.11 |
| ATOM | 10718 | CD | GLU | B | 608 | 93.471 | 67.991 | 64.190 | 1.00 | 38.52 |
| ATOM | 10719 | OE1 | GLU | B | 608 | 93.445 | 67.821 | 62.960 | 1.00 | 40.58 |
| ATOM | 10720 | OE2 | GLU | B | 608 | 94.447 | 67.702 | 64.929 | 1.00 | 40.81 |
| ATOM | 10721 | N | ALA | B | 609 | 89.616 | 65.907 | 65.666 | 1.00 | 29.75 |
| ATOM | 10722 | CA | ALA | B | 609 | 89.602 | 65.008 | 66.815 | 1.00 | 27.89 |
| ATOM | 10723 | C | ALA | B | 609 | 89.757 | 63.593 | 66.372 | 1.00 | 28.03 |
| ATOM | 10724 | O | ALA | B | 609 | 90.522 | 62.823 | 66.983 | 1.00 | 27.07 |
| ATOM | 10725 | CB | ALA | B | 609 | 88.332 | 65.143 | 67.576 | 1.00 | 28.09 |
| ATOM | 10726 | N | ALA | B | 610 | 88.980 | 63.224 | 65.341 | 1.00 | 29.31 |
| ATOM | 10727 | CA | ALA | B | 610 | 89.038 | 61.882 | 64.796 | 1.00 | 30.46 |
| ATOM | 10728 | C | ALA | B | 610 | 90.447 | 61.590 | 64.239 | 1.00 | 31.96 |
| ATOM | 10729 | O | ALA | B | 610 | 90.983 | 60.499 | 64.393 | 1.00 | 30.76 |
| ATOM | 10730 | CB | ALA | B | 610 | 87.993 | 61.728 | 63.725 | 1.00 | 31.13 |
| ATOM | 10731 | N | ARG | B | 611 | 91.047 | 62.569 | 63.576 | 1.00 | 34.38 |
| ATOM | 10732 | CA | ARG | B | 611 | 92.448 | 62.417 | 63.173 | 1.00 | 36.62 |
| ATOM | 10733 | C | ARG | B | 611 | 93.359 | 62.083 | 64.363 | 1.00 | 37.55 |
| ATOM | 10734 | O | ARG | B | 611 | 94.146 | 61.132 | 64.319 | 1.00 | 37.49 |
| ATOM | 10735 | CB | ARG | B | 611 | 92.975 | 63.686 | 62.556 | 1.00 | 37.15 |
| ATOM | 10736 | CG | ARG | B | 611 | 92.409 | 64.030 | 61.232 | 1.00 | 39.17 |
| ATOM | 10737 | CD | ARG | B | 611 | 93.246 | 65.083 | 60.505 | 1.00 | 41.10 |
| ATOM | 10738 | NE | ARG | B | 611 | 92.638 | 65.476 | 59.242 | 1.00 | 43.54 |
| ATOM | 10739 | CZ | ARG | B | 611 | 92.682 | 64.741 | 58.142 | 1.00 | 47.60 |
| ATOM | 10740 | NH1 | ARG | B | 611 | 93.313 | 63.552 | 58.139 | 1.00 | 46.75 |
| ATOM | 10741 | NH2 | ARG | B | 611 | 92.104 | 65.195 | 57.030 | 1.00 | 49.60 |
| ATOM | 10742 | N | GLN | B | 612 | 93.282 | 62.871 | 65.418 | 1.00 | 38.42 |
| ATOM | 10743 | CA | GLN | B | 612 | 94.140 | 62.595 | 66.567 | 1.00 | 39.96 |
| ATOM | 10744 | C | GLN | B | 612 | 93.799 | 61.266 | 67.182 | 1.00 | 41.27 |
| ATOM | 10745 | O | GLN | B | 612 | 94.711 | 60.510 | 67.554 | 1.00 | 42.50 |
| ATOM | 10746 | CB | GLN | B | 612 | 94.067 | 63.688 | 67.613 | 1.00 | 40.19 |
| ATOM | 10747 | CG | GLN | B | 612 | 94.906 | 64.871 | 67.197 | 1.00 | 40.92 |
| ATOM | 10748 | CD | GLN | B | 612 | 95.099 | 65.864 | 68.276 | 1.00 | 40.60 |
| ATOM | 10749 | OE1 | GLN | B | 612 | 95.153 | 65.511 | 69.446 | 1.00 | 40.23 |
| ATOM | 10750 | NE2 | GLN | B | 612 | 95.204 | 67.127 | 67.893 | 1.00 | 41.14 |
| ATOM | 10751 | N | PHE | B | 613 | 92.515 | 60.923 | 67.245 | 1.00 | 41.37 |
| ATOM | 10752 | CA | PHE | B | 613 | 92.180 | 59.645 | 67.835 | 1.00 | 41.93 |
| ATOM | 10753 | C | PHE | B | 613 | 92.852 | 58.529 | 67.056 | 1.00 | 42.81 |
| ATOM | 10754 | O | PHE | B | 613 | 93.341 | 57.564 | 67.655 | 1.00 | 42.45 |
| ATOM | 10755 | CB | PHE | B | 613 | 90.664 | 59.428 | 67.915 | 1.00 | 42.06 |
| ATOM | 10756 | CG | PHE | B | 613 | 89.963 | 60.342 | 68.897 | 1.00 | 40.97 |
| ATOM | 10757 | CD1 | PHE | B | 613 | 90.580 | 60.723 | 70.086 | 1.00 | 39.82 |
| ATOM | 10758 | CD2 | PHE | B | 613 | 88.675 | 60.807 | 68.625 | 1.00 | 38.60 |
| ATOM | 10759 | CE1 | PHE | B | 613 | 89.934 | 61.556 | 70.977 | 1.00 | 38.32 |
| ATOM | 10760 | CE2 | PHE | B | 613 | 88.019 | 61.621 | 69.504 | 1.00 | 37.41 |
| ATOM | 10761 | CZ | PHE | B | 613 | 88.649 | 62.012 | 70.690 | 1.00 | 38.28 |
| ATOM | 10762 | N | SER | B | 614 | 92.864 | 58.671 | 65.732 | 1.00 | 44.37 |
| ATOM | 10763 | CA | SER | B | 614 | 93.507 | 57.716 | 64.813 | 1.00 | 46.26 |
| ATOM | 10764 | C | SER | B | 614 | 94.969 | 57.460 | 65.102 | 1.00 | 47.54 |
| ATOM | 10765 | O | SER | B | 614 | 95.479 | 56.382 | 64.779 | 1.00 | 47.97 |
| ATOM | 10766 | CB | SER | B | 614 | 93.457 | 58.219 | 63.379 | 1.00 | 46.05 |
| ATOM | 10767 | OG | SER | B | 614 | 92.122 | 58.265 | 62.954 | 1.00 | 47.57 |
| ATOM | 10768 | N | LYS | B | 615 | 95.644 | 58.456 | 65.672 | 1.00 | 48.47 |
| ATOM | 10769 | CA | LYS | B | 615 | 97.060 | 58.323 | 65.981 | 1.00 | 49.77 |
| ATOM | 10770 | C | LYS | B | 615 | 97.262 | 57.757 | 67.375 | 1.00 | 49.54 |
| ATOM | 10771 | O | LYS | B | 615 | 98.340 | 57.901 | 67.947 | 1.00 | 50.00 |
| ATOM | 10772 | CB | LYS | B | 615 | 97.764 | 59.679 | 65.921 | 1.00 | 49.86 |
| ATOM | 10773 | CG | LYS | B | 615 | 97.995 | 60.231 | 64.539 | 1.00 | 52.76 |
| ATOM | 10774 | CD | LYS | B | 615 | 99.008 | 61.414 | 64.620 | 1.00 | 55.47 |
| ATOM | 10775 | CE | LYS | B | 615 | 98.927 | 62.385 | 63.415 | 1.00 | 56.51 |
| ATOM | 10776 | NZ | LYS | B | 615 | 99.486 | 63.757 | 63.735 | 1.00 | 55.70 |
| ATOM | 10777 | N | MET | B | 616 | 96.221 | 57.200 | 67.973 | 1.00 | 49.06 |
| ATOM | 10778 | CA | MET | B | 616 | 96.404 | 56.596 | 69.284 | 1.00 | 48.60 |
| ATOM | 10779 | C | MET | B | 616 | 96.638 | 55.111 | 68.946 | 1.00 | 47.93 |
| ATOM | 10780 | O | MET | B | 616 | 95.936 | 54.482 | 68.142 | 1.00 | 48.34 |
| ATOM | 10781 | CB | MET | B | 616 | 95.213 | 56.851 | 70.238 | 1.00 | 49.16 |
| ATOM | 10782 | CG | MET | B | 616 | 95.060 | 58.332 | 70.751 | 1.00 | 49.13 |
| ATOM | 10783 | SD | MET | B | 616 | 93.571 | 58.737 | 71.827 | 1.00 | 49.24 |
| ATOM | 10784 | CE | MET | B | 616 | 93.720 | 60.480 | 71.881 | 1.00 | 43.96 |
| ATOM | 10785 | N | GLY | B | 617 | 97.670 | 54.546 | 69.522 | 1.00 | 46.79 |
| ATOM | 10786 | CA | GLY | B | 617 | 98.040 | 53.204 | 69.131 | 1.00 | 45.51 |
| ATOM | 10787 | C | GLY | B | 617 | 96.886 | 52.233 | 69.138 | 1.00 | 43.75 |
| ATOM | 10788 | O | GLY | B | 617 | 97.038 | 51.139 | 68.590 | 1.00 | 43.25 |
| ATOM | 10789 | N | PHE | B | 618 | 95.742 | 52.621 | 69.721 | 1.00 | 41.77 |
| ATOM | 10790 | CA | PHE | B | 618 | 94.635 | 51.677 | 69.889 | 1.00 | 40.09 |
| ATOM | 10791 | C | PHE | B | 618 | 93.398 | 51.831 | 68.974 | 1.00 | 39.67 |
| ATOM | 10792 | O | PHE | B | 618 | 92.412 | 51.092 | 69.122 | 1.00 | 38.13 |
| ATOM | 10793 | CB | PHE | B | 618 | 94.238 | 51.590 | 71.361 | 1.00 | 39.95 |
| ATOM | 10794 | CG | PHE | B | 618 | 93.787 | 52.894 | 71.975 | 1.00 | 40.73 |
| ATOM | 10795 | CD1 | PHE | B | 618 | 92.480 | 53.300 | 71.882 | 1.00 | 38.86 |
| ATOM | 10796 | CD2 | PHE | B | 618 | 94.678 | 53.689 | 72.692 | 1.00 | 42.73 |
| ATOM | 10797 | CE1 | PHE | B | 618 | 92.052 | 54.469 | 72.451 | 1.00 | 39.59 |
| ATOM | 10798 | CE2 | PHE | B | 618 | 94.261 | 54.881 | 73.272 | 1.00 | 41.95 |
| ATOM | 10799 | CZ | PHE | B | 618 | 92.936 | 55.267 | 73.161 | 1.00 | 42.27 |
| ATOM | 10800 | N | VAL | B | 619 | 93.429 | 52.750 | 68.019 | 1.00 | 39.25 |
| ATOM | 10801 | CA | VAL | B | 619 | 92.303 | 52.791 | 67.094 | 1.00 | 39.86 |
| ATOM | 10802 | C | VAL | B | 619 | 92.742 | 52.435 | 65.686 | 1.00 | 39.43 |
| ATOM | 10803 | O | VAL | B | 619 | 93.769 | 52.868 | 65.199 | 1.00 | 39.46 |
| ATOM | 10804 | CB | VAL | B | 619 | 91.497 | 54.087 | 67.092 | 1.00 | 39.76 |
| ATOM | 10805 | CG1 | VAL | B | 619 | 91.676 | 54.839 | 68.343 | 1.00 | 41.05 |
| ATOM | 10806 | CG2 | VAL | B | 619 | 91.840 | 54.915 | 65.930 | 1.00 | 41.29 |
| ATOM | 10807 | N | ASP | B | 620 | 91.975 | 51.603 | 65.021 | 1.00 | 39.47 |
| ATOM | 10808 | CA | ASP | B | 620 | 92.405 | 51.266 | 63.705 | 1.00 | 39.50 |
| ATOM | 10809 | C | ASP | B | 620 | 91.786 | 52.278 | 62.779 | 1.00 | 38.38 |
| ATOM | 10810 | O | ASP | B | 620 | 90.565 | 52.445 | 62.734 | 1.00 | 37.52 |
| ATOM | 10811 | CB | ASP | B | 620 | 92.112 | 49.807 | 63.361 | 1.00 | 40.45 |
| ATOM | 10812 | CG | ASP | B | 620 | 91.185 | 49.668 | 62.253 | 1.00 | 41.41 |
| ATOM | 10813 | OD1 | ASP | B | 620 | 90.472 | 50.658 | 62.056 | 1.00 | 52.22 |
| ATOM | 10814 | OD2 | ASP | B | 620 | 91.032 | 48.635 | 61.566 | 1.00 | 37.52 |
| ATOM | 10815 | N | ASN | B | 621 | 92.656 | 52.992 | 62.085 | 1.00 | 37.29 |
| ATOM | 10816 | CA | ASN | B | 621 | 92.254 | 54.027 | 61.171 | 1.00 | 37.29 |
| ATOM | 10817 | C | ASN | B | 621 | 91.521 | 53.559 | 59.922 | 1.00 | 36.59 |
| ATOM | 10818 | O | ASN | B | 621 | 91.154 | 54.386 | 59.121 | 1.00 | 36.60 |
| ATOM | 10819 | CB | ASN | B | 621 | 93.471 | 54.840 | 60.754 | 1.00 | 38.33 |
| ATOM | 10820 | CG | ASN | B | 621 | 94.532 | 53.987 | 60.099 | 1.00 | 40.06 |
| ATOM | 10821 | OD1 | ASN | B | 621 | 95.711 | 54.332 | 60.099 | 1.00 | 45.02 |
| ATOM | 10822 | ND2 | ASN | B | 621 | 94.128 | 52.832 | 59.596 | 1.00 | 41.57 |
| ATOM | 10823 | N | LYS | B | 622 | 91.317 | 52.258 | 59.735 | 1.00 | 36.48 |
| ATOM | 10824 | CA | LYS | B | 622 | 90.550 | 51.768 | 58.560 | 1.00 | 36.62 |
| ATOM | 10825 | C | LYS | B | 622 | 89.068 | 51.682 | 58.889 | 1.00 | 34.86 |
| ATOM | 10826 | O | LYS | B | 622 | 88.223 | 51.552 | 57.988 | 1.00 | 34.55 |
| ATOM | 10827 | CB | LYS | B | 622 | 90.989 | 50.353 | 58.136 | 1.00 | 37.22 |
| ATOM | 10828 | CG | LYS | B | 622 | 92.528 | 50.136 | 57.901 | 1.00 | 40.89 |
| ATOM | 10829 | CD | LYS | B | 622 | 92.799 | 48.711 | 57.342 | 1.00 | 45.06 |
| ATOM | 10830 | CE | LYS | B | 622 | 93.442 | 47.742 | 58.372 | 1.00 | 46.89 |
| ATOM | 10831 | NZ | LYS | B | 622 | 93.320 | 46.309 | 57.939 | 1.00 | 45.93 |
| ATOM | 10832 | N | ARG | B | 623 | 88.750 | 51.725 | 60.179 | 1.00 | 32.22 |
| ATOM | 10833 | CA | ARG | B | 623 | 87.368 | 51.618 | 60.588 | 1.00 | 31.40 |
| ATOM | 10834 | C | ARG | B | 623 | 86.945 | 52.695 | 61.579 | 1.00 | 30.66 |
| ATOM | 10835 | O | ARG | B | 623 | 86.631 | 52.413 | 62.755 | 1.00 | 30.63 |
| ATOM | 10836 | CB | ARG | B | 623 | 87.083 | 50.232 | 61.147 | 1.00 | 31.13 |
| ATOM | 10837 | CG | ARG | B | 623 | 87.357 | 49.109 | 60.184 | 1.00 | 31.70 |
| ATOM | 10838 | CD | ARG | B | 623 | 87.110 | 47.735 | 60.762 | 1.00 | 32.44 |
| ATOM | 10839 | NE | ARG | B | 623 | 88.102 | 47.366 | 61.741 | 1.00 | 34.08 |
| ATOM | 10840 | CZ | ARG | B | 623 | 87.936 | 46.458 | 62.663 | 1.00 | 35.71 |
| ATOM | 10841 | NH1 | ARG | B | 623 | 86.796 | 45.822 | 62.770 | 1.00 | 37.93 |
| ATOM | 10842 | NH2 | ARG | B | 623 | 88.902 | 46.204 | 63.527 | 1.00 | 38.72 |
| ATOM | 10843 | N | ILE | B | 624 | 86.944 | 53.923 | 61.080 | 1.00 | 29.13 |
| ATOM | 10844 | CA | ILE | B | 624 | 86.370 | 55.044 | 61.773 | 1.00 | 28.86 |
| ATOM | 10845 | C | ILE | B | 624 | 85.078 | 55.496 | 61.104 | 1.00 | 28.02 |
| ATOM | 10846 | O | ILE | B | 624 | 85.050 | 55.766 | 59.897 | 1.00 | 27.56 |
| ATOM | 10847 | CB | ILE | B | 624 | 87.327 | 56.199 | 61.805 | 1.00 | 29.62 |
| ATOM | 10848 | CG1 | ILE | B | 624 | 88.700 | 55.682 | 62.273 | 1.00 | 31.76 |
| ATOM | 10849 | CG2 | ILE | B | 624 | 86.773 | 57.277 | 62.713 | 1.00 | 26.67 |
| ATOM | 10850 | CD1 | ILE | B | 624 | 89.588 | 56.760 | 62.828 | 1.00 | 34.52 |
| ATOM | 10851 | N | ALA | B | 625 | 84.011 | 55.545 | 61.902 | 1.00 | 27.38 |
| ATOM | 10852 | CA | ALA | B | 625 | 82.688 | 55.998 | 61.480 | 1.00 | 26.62 |
| ATOM | 10853 | C | ALA | B | 625 | 82.232 | 57.184 | 62.293 | 1.00 | 26.66 |
| ATOM | 10854 | O | ALA | B | 625 | 82.800 | 57.490 | 63.366 | 1.00 | 26.60 |
| ATOM | 10855 | CB | ALA | B | 625 | 81.694 | 54.880 | 61.663 | 1.00 | 27.16 |
| ATOM | 10856 | N | ILE | B | 626 | 81.165 | 57.826 | 61.832 | 1.00 | 25.62 |
| ATOM | 10857 | CA | ILE | B | 626 | 80.584 | 58.918 | 62.591 | 1.00 | 25.90 |
| ATOM | 10858 | C | ILE | B | 626 | 79.033 | 58.874 | 62.559 | 1.00 | 25.62 |
| ATOM | 10859 | O | ILE | B | 626 | 78.433 | 58.405 | 61.593 | 1.00 | 24.90 |
| ATOM | 10860 | CB | ILE | B | 626 | 81.107 | 60.237 | 62.034 | 1.00 | 25.89 |
| ATOM | 10861 | CG1 | ILE | B | 626 | 80.392 | 61.433 | 62.664 | 1.00 | 26.49 |
| ATOM | 10862 | CG2 | ILE | B | 626 | 80.877 | 60.271 | 60.593 | 1.00 | 25.97 |
| ATOM | 10863 | CD1 | ILE | B | 626 | 81.076 | 62.736 | 62.353 | 1.00 | 26.25 |
| ATOM | 10864 | N | TRP | B | 627 | 78.389 | 59.326 | 63.635 | 1.00 | 25.37 |
| ATOM | 10865 | CA | TRP | B | 627 | 76.932 | 59.378 | 63.642 | 1.00 | 25.54 |
| ATOM | 10866 | C | TRP | B | 627 | 76.355 | 60.378 | 64.609 | 1.00 | 25.72 |
| ATOM | 10867 | O | TRP | B | 627 | 77.024 | 60.856 | 65.563 | 1.00 | 23.56 |
| ATOM | 10868 | CB | TRP | B | 627 | 76.338 | 58.002 | 63.902 | 1.00 | 26.23 |
| ATOM | 10869 | CG | TRP | B | 627 | 75.971 | 57.723 | 65.307 | 1.00 | 26.20 |
| ATOM | 10870 | CD1 | TRP | B | 627 | 76.804 | 57.334 | 66.314 | 1.00 | 27.25 |
| ATOM | 10871 | CD2 | TRP | B | 627 | 74.668 | 57.779 | 65.865 | 1.00 | 28.18 |
| ATOM | 10872 | NE1 | TRP | B | 627 | 76.093 | 57.163 | 67.478 | 1.00 | 30.10 |
| ATOM | 10873 | CE2 | TRP | B | 627 | 74.777 | 57.434 | 67.232 | 1.00 | 29.88 |
| ATOM | 10874 | CE3 | TRP | B | 627 | 73.415 | 58.131 | 65.365 | 1.00 | 30.86 |
| ATOM | 10875 | CZ2 | TRP | B | 627 | 73.693 | 57.402 | 68.075 | 1.00 | 30.30 |
| ATOM | 10876 | CZ3 | TRP | B | 627 | 72.339 | 58.087 | 66.207 | 1.00 | 30.30 |
| ATOM | 10877 | CH2 | TRP | B | 627 | 72.486 | 57.738 | 67.545 | 1.00 | 31.45 |
| ATOM | 10878 | N | GLY | B | 628 | 75.094 | 60.706 | 64.337 | 1.00 | 25.24 |
| ATOM | 10879 | CA | GLY | B | 628 | 74.390 | 61.673 | 65.142 | 1.00 | 25.27 |
| ATOM | 10880 | C | GLY | B | 628 | 72.983 | 61.934 | 64.681 | 1.00 | 24.81 |
| ATOM | 10881 | O | GLY | B | 628 | 72.542 | 61.457 | 63.623 | 1.00 | 26.04 |
| ATOM | 10882 | N | TRP | B | 629 | 72.300 | 62.719 | 65.484 | 1.00 | 24.13 |
| ATOM | 10883 | CA | TRP | B | 629 | 70.876 | 62.978 | 65.343 | 1.00 | 24.48 |
| ATOM | 10884 | C | TRP | B | 629 | 70.629 | 64.466 | 65.431 | 1.00 | 23.61 |
| ATOM | 10885 | O | TRP | B | 629 | 71.149 | 65.111 | 66.328 | 1.00 | 23.92 |
| ATOM | 10886 | CB | TRP | B | 629 | 70.206 | 62.301 | 66.553 | 1.00 | 24.77 |
| ATOM | 10887 | CG | TRP | B | 629 | 68.757 | 61.953 | 66.444 | 1.00 | 24.22 |
| ATOM | 10888 | CD1 | TRP | B | 629 | 67.736 | 62.796 | 66.211 | 1.00 | 22.92 |
| ATOM | 10889 | CD2 | TRP | B | 629 | 68.169 | 60.661 | 66.678 | 1.00 | 23.65 |
| ATOM | 10890 | NE1 | TRP | B | 629 | 66.547 | 62.111 | 66.246 | 1.00 | 24.46 |
| ATOM | 10891 | CE2 | TRP | B | 629 | 66.786 | 60.803 | 66.550 | 1.00 | 22.65 |
| ATOM | 10892 | CE3 | TRP | B | 629 | 68.685 | 59.403 | 66.999 | 1.00 | 24.62 |
| ATOM | 10893 | CZ2 | TRP | B | 629 | 65.904 | 59.756 | 66.709 | 1.00 | 24.04 |
| ATOM | 10894 | CZ3 | TRP | B | 629 | 67.807 | 58.361 | 67.167 | 1.00 | 27.54 |
| ATOM | 10895 | CH2 | TRP | B | 629 | 66.421 | 58.545 | 67.022 | 1.00 | 24.47 |
| ATOM | 10896 | N | SER | B | 630 | 69.846 | 65.011 | 64.518 | 1.00 | 23.06 |
| ATOM | 10897 | CA | SER | B | 630 | 69.485 | 66.425 | 64.581 | 1.00 | 23.38 |
| ATOM | 10898 | C | SER | B | 630 | 70.723 | 67.254 | 64.264 | 1.00 | 23.53 |
| ATOM | 10899 | O | SER | B | 630 | 71.281 | 67.062 | 63.174 | 1.00 | 24.59 |
| ATOM | 10900 | CB | SER | B | 630 | 68.828 | 66.734 | 65.925 | 1.00 | 23.43 |
| ATOM | 10901 | OG | SER | B | 630 | 68.001 | 67.853 | 65.825 | 1.00 | 24.43 |
| ATOM | 10902 | N | TYR | B | 631 | 71.184 | 68.138 | 65.153 | 1.00 | 22.69 |
| ATOM | 10903 | CA | TYR | B | 631 | 72.402 | 68.853 | 64.874 | 1.00 | 21.94 |
| ATOM | 10904 | C | TYR | B | 631 | 73.504 | 67.820 | 64.589 | 1.00 | 21.26 |
| ATOM | 10905 | O | TYR | B | 631 | 74.304 | 67.972 | 63.686 | 1.00 | 18.51 |
| ATOM | 10906 | CB | TYR | B | 631 | 72.797 | 69.855 | 65.987 | 1.00 | 21.15 |
| ATOM | 10907 | CG | TYR | B | 631 | 73.646 | 70.999 | 65.477 | 1.00 | 22.05 |
| ATOM | 10908 | CD1 | TYR | B | 631 | 74.992 | 70.829 | 65.194 | 1.00 | 26.96 |
| ATOM | 10909 | CD2 | TYR | B | 631 | 73.118 | 72.225 | 65.281 | 1.00 | 25.64 |
| ATOM | 10910 | CE1 | TYR | B | 631 | 75.780 | 71.878 | 64.717 | 1.00 | 24.46 |
| ATOM | 10911 | CE2 | TYR | B | 631 | 73.889 | 73.281 | 64.805 | 1.00 | 26.67 |
| ATOM | 10912 | CZ | TYR | B | 631 | 75.213 | 73.090 | 64.515 | 1.00 | 24.34 |
| ATOM | 10913 | OH | TYR | B | 631 | 75.955 | 74.140 | 64.047 | 1.00 | 24.92 |
| ATOM | 10914 | N | GLY | B | 632 | 73.511 | 66.743 | 65.366 | 1.00 | 21.64 |
| ATOM | 10915 | CA | GLY | B | 632 | 74.465 | 65.679 | 65.120 | 1.00 | 21.85 |
| ATOM | 10916 | C | GLY | B | 632 | 74.338 | 65.083 | 63.728 | 1.00 | 23.13 |
| ATOM | 10917 | O | GLY | B | 632 | 75.323 | 64.583 | 63.184 | 1.00 | 22.77 |
| ATOM | 10918 | N | GLY | B | 633 | 73.138 | 65.138 | 63.139 | 1.00 | 23.35 |
| ATOM | 10919 | CA | GLY | B | 633 | 72.944 | 64.608 | 61.800 | 1.00 | 23.84 |
| ATOM | 10920 | C | GLY | B | 633 | 73.525 | 65.537 | 60.736 | 1.00 | 23.80 |
| ATOM | 10921 | O | GLY | B | 633 | 74.095 | 65.114 | 59.758 | 1.00 | 23.33 |
| ATOM | 10922 | N | TYR | B | 634 | 73.344 | 66.828 | 60.937 | 1.00 | 24.33 |
| ATOM | 10923 | CA | TYR | B | 634 | 73.911 | 67.831 | 60.064 | 1.00 | 24.48 |
| ATOM | 10924 | C | TYR | B | 634 | 75.442 | 67.723 | 60.070 | 1.00 | 25.35 |
| ATOM | 10925 | O | TYR | B | 634 | 76.078 | 67.661 | 58.998 | 1.00 | 24.10 |
| ATOM | 10926 | CB | TYR | B | 634 | 73.485 | 69.181 | 60.609 | 1.00 | 24.24 |
| ATOM | 10927 | CG | TYR | B | 634 | 74.110 | 70.393 | 59.949 | 1.00 | 26.45 |
| ATOM | 10928 | CD1 | TYR | B | 634 | 73.843 | 70.714 | 58.629 | 1.00 | 25.37 |
| ATOM | 10929 | CD2 | TYR | B | 634 | 74.927 | 71.248 | 60.677 | 1.00 | 25.94 |
| ATOM | 10930 | CE1 | TYR | B | 634 | 74.399 | 71.834 | 58.047 | 1.00 | 27.88 |
| ATOM | 10931 | CE2 | TYR | B | 634 | 75.490 | 72.378 | 60.109 | 1.00 | 26.59 |
| ATOM | 10932 | CZ | TYR | B | 634 | 75.234 | 72.675 | 58.806 | 1.00 | 29.36 |
| ATOM | 10933 | OH | TYR | B | 634 | 75.812 | 73.802 | 58.241 | 1.00 | 28.75 |
| ATOM | 10934 | N | VAL | B | 635 | 76.025 | 67.702 | 61.280 | 1.00 | 24.81 |
| ATOM | 10935 | CA | VAL | B | 635 | 77.487 | 67.681 | 61.406 | 1.00 | 25.08 |
| ATOM | 10936 | C | VAL | B | 635 | 78.055 | 66.432 | 60.774 | 1.00 | 24.69 |
| ATOM | 10937 | O | VAL | B | 635 | 79.019 | 66.512 | 60.003 | 1.00 | 23.47 |
| ATOM | 10938 | CB | VAL | B | 635 | 77.966 | 67.859 | 62.895 | 1.00 | 24.90 |
| ATOM | 10939 | CG1 | VAL | B | 635 | 79.469 | 67.653 | 63.033 | 1.00 | 25.35 |
| ATOM | 10940 | CG2 | VAL | B | 635 | 77.553 | 69.233 | 63.354 | 1.00 | 26.03 |
| ATOM | 10941 | N | THR | B | 636 | 77.438 | 65.292 | 61.063 | 1.00 | 25.24 |
| ATOM | 10942 | CA | THR | B | 636 | 77.819 | 64.024 | 60.439 | 1.00 | 25.51 |
| ATOM | 10943 | C | THR | B | 636 | 77.768 | 64.125 | 58.931 | 1.00 | 26.59 |
| ATOM | 10944 | O | THR | B | 636 | 78.602 | 63.524 | 58.224 | 1.00 | 28.02 |
| ATOM | 10945 | CB | THR | B | 636 | 76.827 | 62.951 | 60.801 | 1.00 | 25.92 |
| ATOM | 10946 | OG1 | THR | B | 636 | 76.883 | 62.701 | 62.188 | 1.00 | 24.04 |
| ATOM | 10947 | CG2 | THR | B | 636 | 77.181 | 61.551 | 60.119 | 1.00 | 25.77 |
| ATOM | 10948 | N | SER | B | 637 | 76.774 | 64.848 | 58.429 | 1.00 | 26.02 |
| ATOM | 10949 | CA | SER | B | 637 | 76.591 | 64.956 | 56.999 | 1.00 | 26.59 |
| ATOM | 10950 | C | SER | B | 637 | 77.638 | 65.867 | 56.384 | 1.00 | 26.80 |
| ATOM | 10951 | O | SER | B | 637 | 78.242 | 65.539 | 55.354 | 1.00 | 26.72 |
| ATOM | 10952 | CB | SER | B | 637 | 75.170 | 65.405 | 56.663 | 1.00 | 26.60 |
| ATOM | 10953 | OG | SER | B | 637 | 74.215 | 64.389 | 57.024 | 1.00 | 26.80 |
| ATOM | 10954 | N | MET | B | 638 | 77.882 | 66.985 | 57.042 | 1.00 | 27.30 |
| ATOM | 10955 | CA | MET | B | 638 | 78.882 | 67.937 | 56.597 | 1.00 | 27.13 |
| ATOM | 10956 | C | MET | B | 638 | 80.245 | 67.261 | 56.653 | 1.00 | 27.34 |
| ATOM | 10957 | O | MET | B | 638 | 81.064 | 67.389 | 55.746 | 1.00 | 27.78 |
| ATOM | 10958 | CB | MET | B | 638 | 78.822 | 69.183 | 57.477 | 1.00 | 27.08 |
| ATOM | 10959 | CG | MET | B | 638 | 77.519 | 69.960 | 57.350 | 1.00 | 26.30 |
| ATOM | 10960 | SD | MET | B | 638 | 77.261 | 70.726 | 55.761 | 1.00 | 28.61 |
| ATOM | 10961 | CE | MET | B | 638 | 78.254 | 72.143 | 55.853 | 1.00 | 26.11 |
| ATOM | 10962 | N | VAL | B | 639 | 80.480 | 66.486 | 57.689 | 1.00 | 27.29 |
| ATOM | 10963 | CA | VAL | B | 639 | 81.743 | 65.787 | 57.804 | 1.00 | 27.36 |
| ATOM | 10964 | C | VAL | B | 639 | 81.896 | 64.778 | 56.687 | 1.00 | 27.81 |
| ATOM | 10965 | O | VAL | B | 639 | 82.939 | 64.720 | 56.049 | 1.00 | 27.30 |
| ATOM | 10966 | CB | VAL | B | 639 | 81.870 | 65.024 | 59.092 | 1.00 | 26.81 |
| ATOM | 10967 | CG1 | VAL | B | 639 | 83.004 | 64.058 | 58.981 | 1.00 | 26.22 |
| ATOM | 10968 | CG2 | VAL | B | 639 | 82.091 | 65.966 | 60.253 | 1.00 | 26.28 |
| ATOM | 10969 | N | LEU | B | 640 | 80.875 | 63.961 | 56.467 | 1.00 | 27.96 |
| ATOM | 10970 | CA | LEU | B | 640 | 80.961 | 62.971 | 55.410 | 1.00 | 28.54 |
| ATOM | 10971 | C | LEU | B | 640 | 81.085 | 63.628 | 54.037 | 1.00 | 28.94 |
| ATOM | 10972 | O | LEU | B | 640 | 81.571 | 63.015 | 53.107 | 1.00 | 29.26 |
| ATOM | 10973 | CB | LEU | B | 640 | 79.754 | 62.024 | 55.416 | 1.00 | 28.12 |
| ATOM | 10974 | CG | LEU | B | 640 | 79.710 | 61.126 | 56.631 | 1.00 | 27.04 |
| ATOM | 10975 | CD1 | LEU | B | 640 | 78.400 | 60.545 | 56.665 | 1.00 | 27.15 |
| ATOM | 10976 | CD2 | LEU | B | 640 | 80.752 | 60.066 | 56.551 | 1.00 | 27.07 |
| ATOM | 10977 | N | GLY | B | 641 | 80.615 | 64.858 | 53.922 | 1.00 | 28.92 |
| ATOM | 10978 | CA | GLY | B | 641 | 80.716 | 65.577 | 52.690 | 1.00 | 29.39 |
| ATOM | 10979 | C | GLY | B | 641 | 81.948 | 66.473 | 52.614 | 1.00 | 29.96 |
| ATOM | 10980 | O | GLY | B | 641 | 82.051 | 67.295 | 51.696 | 1.00 | 29.05 |
| ATOM | 10981 | N | SER | B | 642 | 82.873 | 66.316 | 53.560 | 1.00 | 29.53 |
| ATOM | 10982 | CA | SER | B | 642 | 84.017 | 67.187 | 53.620 | 1.00 | 29.91 |
| ATOM | 10983 | C | SER | B | 642 | 85.170 | 66.768 | 52.678 | 1.00 | 31.18 |
| ATOM | 10984 | O | SER | B | 642 | 85.976 | 67.607 | 52.287 | 1.00 | 31.66 |
| ATOM | 10985 | CB | SER | B | 642 | 84.582 | 67.206 | 55.028 | 1.00 | 29.68 |
| ATOM | 10986 | OG | SER | B | 642 | 85.219 | 65.956 | 55.281 | 1.00 | 28.01 |
| ATOM | 10987 | N | GLY | B | 643 | 85.259 | 65.483 | 52.364 | 1.00 | 32.16 |
| ATOM | 10988 | CA | GLY | B | 643 | 86.341 | 64.941 | 51.565 | 1.00 | 33.04 |
| ATOM | 10989 | C | GLY | B | 643 | 87.582 | 64.561 | 52.375 | 1.00 | 33.77 |
| ATOM | 10990 | O | GLY | B | 643 | 88.597 | 64.215 | 51.814 | 1.00 | 33.62 |
| ATOM | 10991 | N | SER | B | 644 | 87.463 | 64.579 | 53.695 | 1.00 | 34.07 |
| ATOM | 10992 | CA | SER | B | 644 | 88.615 | 64.404 | 54.595 | 1.00 | 34.04 |
| ATOM | 10993 | C | SER | B | 644 | 89.333 | 63.079 | 54.409 | 1.00 | 34.10 |
| ATOM | 10994 | O | SER | B | 644 | 90.552 | 62.990 | 54.610 | 1.00 | 34.02 |
| ATOM | 10995 | CB | SER | B | 644 | 88.167 | 64.519 | 56.076 | 1.00 | 33.03 |
| ATOM | 10996 | OG | SER | B | 644 | 87.585 | 63.294 | 56.498 | 1.00 | 31.58 |
| ATOM | 10997 | N | GLY | B | 645 | 88.569 | 62.047 | 54.075 | 1.00 | 34.16 |
| ATOM | 10998 | CA | GLY | B | 645 | 89.112 | 60.707 | 53.898 | 1.00 | 33.94 |
| ATOM | 10999 | C | GLY | B | 645 | 89.207 | 59.961 | 55.203 | 1.00 | 34.82 |
| ATOM | 11000 | O | GLY | B | 645 | 89.521 | 58.765 | 55.245 | 1.00 | 35.73 |
| ATOM | 11001 | N | VAL | B | 646 | 88.917 | 60.647 | 56.300 | 1.00 | 34.33 |
| ATOM | 11002 | CA | VAL | B | 646 | 89.080 | 60.025 | 57.605 | 1.00 | 33.90 |
| ATOM | 11003 | C | VAL | B | 646 | 87.989 | 59.008 | 57.932 | 1.00 | 33.16 |
| ATOM | 11004 | O | VAL | B | 646 | 88.235 | 58.021 | 58.615 | 1.00 | 32.54 |
| ATOM | 11005 | CB | VAL | B | 646 | 89.111 | 61.129 | 58.672 | 1.00 | 34.58 |
| ATOM | 11006 | CG1 | VAL | B | 646 | 89.123 | 60.546 | 60.086 | 1.00 | 34.63 |
| ATOM | 11007 | CG2 | VAL | B | 646 | 90.338 | 62.054 | 58.407 | 1.00 | 34.31 |
| ATOM | 11008 | N | PHE | B | 647 | 86.780 | 59.217 | 57.433 | 1.00 | 32.43 |
| ATOM | 11009 | CA | PHE | B | 647 | 85.668 | 58.345 | 57.828 | 1.00 | 32.12 |
| ATOM | 11010 | C | PHE | B | 647 | 85.239 | 57.394 | 56.756 | 1.00 | 31.73 |
| ATOM | 11011 | O | PHE | B | 647 | 85.004 | 57.789 | 55.670 | 1.00 | 32.31 |
| ATOM | 11012 | CB | PHE | B | 647 | 84.484 | 59.215 | 58.241 | 1.00 | 32.01 |
| ATOM | 11013 | CG | PHE | B | 647 | 84.819 | 60.173 | 59.325 | 1.00 | 31.30 |
| ATOM | 11014 | CD1 | PHE | B | 647 | 85.398 | 61.391 | 59.037 | 1.00 | 31.77 |
| ATOM | 11015 | CD2 | PHE | B | 647 | 84.561 | 59.863 | 60.643 | 1.00 | 31.08 |
| ATOM | 11016 | CE1 | PHE | B | 647 | 85.716 | 62.284 | 60.066 | 1.00 | 28.09 |
| ATOM | 11017 | CE2 | PHE | B | 647 | 84.894 | 60.754 | 61.656 | 1.00 | 28.81 |
| ATOM | 11018 | CZ | PHE | B | 647 | 85.477 | 61.944 | 61.353 | 1.00 | 27.39 |
| ATOM | 11019 | N | LYS | B | 648 | 85.096 | 56.127 | 57.090 | 1.00 | 32.24 |
| ATOM | 11020 | CA | LYS | B | 648 | 84.673 | 55.125 | 56.138 | 1.00 | 31.64 |
| ATOM | 11021 | C | LYS | B | 648 | 83.168 | 55.162 | 55.964 | 1.00 | 32.05 |
| ATOM | 11022 | O | LYS | B | 648 | 82.679 | 54.875 | 54.897 | 1.00 | 30.31 |
| ATOM | 11023 | CB | LYS | B | 648 | 85.045 | 53.751 | 56.660 | 1.00 | 32.05 |
| ATOM | 11024 | CG | LYS | B | 648 | 84.533 | 52.555 | 55.852 | 1.00 | 31.04 |
| ATOM | 11025 | CD | LYS | B | 648 | 85.342 | 51.367 | 56.257 | 1.00 | 32.31 |
| ATOM | 11026 | CE | LYS | B | 648 | 84.668 | 50.064 | 56.104 | 1.00 | 35.71 |
| ATOM | 11027 | NZ | LYS | B | 648 | 84.285 | 49.810 | 54.731 | 1.00 | 39.53 |
| ATOM | 11028 | N | CYS | B | 649 | 82.422 | 55.500 | 57.013 | 1.00 | 31.54 |
| ATOM | 11029 | CA | CYS | B | 649 | 80.966 | 55.524 | 56.893 | 1.00 | 32.41 |
| ATOM | 11030 | C | CYS | B | 649 | 80.289 | 56.334 | 57.988 | 1.00 | 31.12 |
| ATOM | 11031 | O | CYS | B | 649 | 80.907 | 56.668 | 58.967 | 1.00 | 30.91 |
| ATOM | 11032 | CB | CYS | B | 649 | 80.428 | 54.118 | 56.967 | 1.00 | 32.74 |
| ATOM | 11033 | SG | CYS | B | 649 | 80.676 | 53.369 | 58.582 | 1.00 | 37.84 |
| ATOM | 11034 | N | GLY | B | 650 | 79.015 | 56.663 | 57.789 | 1.00 | 30.42 |
| ATOM | 11035 | CA | GLY | B | 650 | 78.238 | 57.378 | 58.787 | 1.00 | 29.72 |
| ATOM | 11036 | C | GLY | B | 650 | 76.727 | 57.321 | 58.638 | 1.00 | 28.44 |
| ATOM | 11037 | O | GLY | B | 650 | 76.193 | 56.973 | 57.571 | 1.00 | 27.80 |
| ATOM | 11038 | N | ILE | B | 651 | 76.055 | 57.697 | 59.731 | 1.00 | 26.95 |
| ATOM | 11039 | CA | ILE | B | 651 | 74.614 | 57.677 | 59.821 | 1.00 | 25.90 |
| ATOM | 11040 | C | ILE | B | 651 | 74.102 | 59.005 | 60.319 | 1.00 | 25.19 |
| ATOM | 11041 | O | ILE | B | 651 | 74.453 | 59.425 | 61.392 | 1.00 | 24.71 |
| ATOM | 11042 | CB | ILE | B | 651 | 74.137 | 56.626 | 60.817 | 1.00 | 25.72 |
| ATOM | 11043 | CG1 | ILE | B | 651 | 74.768 | 55.285 | 60.574 | 1.00 | 24.38 |
| ATOM | 11044 | CG2 | ILE | B | 651 | 72.594 | 56.507 | 60.774 | 1.00 | 26.85 |
| ATOM | 11045 | CD1 | ILE | B | 651 | 74.579 | 54.338 | 61.740 | 1.00 | 26.43 |
| ATOM | 11046 | N | ALA | B | 652 | 73.254 | 59.651 | 59.538 | 1.00 | 24.38 |
| ATOM | 11047 | CA | ALA | B | 652 | 72.638 | 60.873 | 59.953 | 1.00 | 24.14 |
| ATOM | 11048 | C | ALA | B | 652 | 71.128 | 60.638 | 60.160 | 1.00 | 24.49 |
| ATOM | 11049 | O | ALA | B | 652 | 70.425 | 60.230 | 59.236 | 1.00 | 24.20 |
| ATOM | 11050 | CB | ALA | B | 652 | 72.879 | 61.919 | 58.931 | 1.00 | 23.84 |
| ATOM | 11051 | N | VAL | B | 653 | 70.638 | 60.921 | 61.361 | 1.00 | 24.13 |
| ATOM | 11052 | CA | VAL | B | 653 | 69.238 | 60.712 | 61.690 | 1.00 | 23.22 |
| ATOM | 11053 | C | VAL | B | 653 | 68.633 | 62.063 | 61.885 | 1.00 | 22.38 |
| ATOM | 11054 | O | VAL | B | 653 | 69.149 | 62.864 | 62.635 | 1.00 | 20.78 |
| ATOM | 11055 | CB | VAL | B | 653 | 69.077 | 59.917 | 62.993 | 1.00 | 23.83 |
| ATOM | 11056 | CG1 | VAL | B | 653 | 67.599 | 59.668 | 63.306 | 1.00 | 25.62 |
| ATOM | 11057 | CG2 | VAL | B | 653 | 69.837 | 58.616 | 62.932 | 1.00 | 23.64 |
| ATOM | 11058 | N | ALA | B | 654 | 67.538 | 62.300 | 61.172 | 1.00 | 22.39 |
| ATOM | 11059 | CA | ALA | B | 654 | 66.783 | 63.561 | 61.200 | 1.00 | 22.03 |
| ATOM | 11060 | C | ALA | B | 654 | 67.645 | 64.821 | 61.182 | 1.00 | 22.07 |
| ATOM | 11061 | O | ALA | B | 654 | 67.473 | 65.774 | 61.948 | 1.00 | 22.17 |
| ATOM | 11062 | CB | ALA | B | 654 | 65.812 | 63.558 | 62.309 | 1.00 | 22.31 |
| ATOM | 11063 | N | PRO | B | 655 | 68.523 | 64.883 | 60.208 | 1.00 | 22.10 |
| ATOM | 11064 | CA | PRO | B | 655 | 69.455 | 66.002 | 60.119 | 1.00 | 22.53 |
| ATOM | 11065 | C | PRO | B | 655 | 68.845 | 67.297 | 59.654 | 1.00 | 23.04 |
| ATOM | 11066 | O | PRO | B | 655 | 67.907 | 67.279 | 58.873 | 1.00 | 22.83 |
| ATOM | 11067 | CB | PRO | B | 655 | 70.425 | 65.536 | 59.034 | 1.00 | 21.39 |
| ATOM | 11068 | CG | PRO | B | 655 | 69.488 | 64.812 | 58.095 | 1.00 | 22.64 |
| ATOM | 11069 | CD | PRO | B | 655 | 68.706 | 63.928 | 59.102 | 1.00 | 22.27 |
| ATOM | 11070 | N | VAL | B | 656 | 69.387 | 68.415 | 60.130 | 1.00 | 23.35 |
| ATOM | 11071 | CA | VAL | B | 656 | 69.122 | 69.685 | 59.474 | 1.00 | 23.76 |
| ATOM | 11072 | C | VAL | B | 656 | 69.979 | 69.620 | 58.218 | 1.00 | 22.91 |
| ATOM | 11073 | O | VAL | B | 656 | 71.054 | 69.072 | 58.281 | 1.00 | 21.69 |
| ATOM | 11074 | CB | VAL | B | 656 | 69.627 | 70.838 | 60.331 | 1.00 | 24.87 |
| ATOM | 11075 | CG1 | VAL | B | 656 | 69.783 | 72.094 | 59.487 | 1.00 | 25.30 |
| ATOM | 11076 | CG2 | VAL | B | 656 | 68.687 | 71.073 | 61.522 | 1.00 | 25.10 |
| ATOM | 11077 | N | SER | B | 657 | 69.520 | 70.141 | 57.088 | 1.00 | 23.02 |
| ATOM | 11078 | CA | SER | B | 657 | 70.337 | 70.107 | 55.856 | 1.00 | 23.47 |
| ATOM | 11079 | C | SER | B | 657 | 70.642 | 71.474 | 55.351 | 1.00 | 23.21 |
| ATOM | 11080 | O | SER | B | 657 | 71.584 | 71.633 | 54.620 | 1.00 | 23.83 |
| ATOM | 11081 | CB | SER | B | 657 | 69.650 | 69.306 | 54.731 | 1.00 | 23.46 |
| ATOM | 11082 | OG | SER | B | 657 | 68.412 | 69.873 | 54.391 | 1.00 | 24.11 |
| ATOM | 11083 | N | ARG | B | 658 | 69.894 | 72.470 | 55.816 | 1.00 | 23.91 |
| ATOM | 11084 | CA | ARG | B | 658 | 69.950 | 73.807 | 55.268 | 1.00 | 24.20 |
| ATOM | 11085 | C | ARG | B | 658 | 69.214 | 74.683 | 56.216 | 1.00 | 24.78 |
| ATOM | 11086 | O | ARG | B | 658 | 68.035 | 74.396 | 56.586 | 1.00 | 24.47 |
| ATOM | 11087 | CB | ARG | B | 658 | 69.275 | 73.816 | 53.920 | 1.00 | 26.18 |
| ATOM | 11088 | CG | ARG | B | 658 | 69.037 | 75.156 | 53.338 | 1.00 | 27.15 |
| ATOM | 11089 | CD | ARG | B | 658 | 68.373 | 75.065 | 52.046 | 1.00 | 29.42 |
| ATOM | 11090 | NE | ARG | B | 658 | 68.658 | 76.130 | 51.134 | 1.00 | 32.58 |
| ATOM | 11091 | CZ | ARG | B | 658 | 67.776 | 76.999 | 50.687 | 1.00 | 38.45 |
| ATOM | 11092 | NH1 | ARG | B | 658 | 66.518 | 76.975 | 51.125 | 1.00 | 41.84 |
| ATOM | 11093 | NH2 | ARG | B | 658 | 68.153 | 77.918 | 49.791 | 1.00 | 37.18 |
| ATOM | 11094 | N | TRP | B | 659 | 69.888 | 75.755 | 56.624 | 1.00 | 24.24 |
| ATOM | 11095 | CA | TRP | B | 659 | 69.449 | 76.533 | 57.741 | 1.00 | 25.17 |
| ATOM | 11096 | C | TRP | B | 659 | 68.193 | 77.296 | 57.439 | 1.00 | 25.41 |
| ATOM | 11097 | O | TRP | B | 659 | 67.378 | 77.516 | 58.338 | 1.00 | 24.31 |
| ATOM | 11098 | CB | TRP | B | 659 | 70.610 | 77.363 | 58.354 | 1.00 | 25.34 |
| ATOM | 11099 | CG | TRP | B | 659 | 71.507 | 76.474 | 59.089 | 1.00 | 25.05 |
| ATOM | 11100 | CD1 | TRP | B | 659 | 72.773 | 76.130 | 58.761 | 1.00 | 26.68 |
| ATOM | 11101 | CD2 | TRP | B | 659 | 71.194 | 75.758 | 60.275 | 1.00 | 21.37 |
| ATOM | 11102 | NE1 | TRP | B | 659 | 73.260 | 75.222 | 59.662 | 1.00 | 26.82 |
| ATOM | 11103 | CE2 | TRP | B | 659 | 72.300 | 74.975 | 60.597 | 1.00 | 22.41 |
| ATOM | 11104 | CE3 | TRP | B | 659 | 70.073 | 75.687 | 61.091 | 1.00 | 21.36 |
| ATOM | 11105 | CZ2 | TRP | B | 659 | 72.334 | 74.164 | 61.710 | 1.00 | 22.83 |
| ATOM | 11106 | CZ3 | TRP | B | 659 | 70.103 | 74.876 | 62.200 | 1.00 | 18.75 |
| ATOM | 11107 | CH2 | TRP | B | 659 | 71.201 | 74.125 | 62.494 | 1.00 | 20.87 |
| ATOM | 11108 | N | GLU | B | 660 | 67.951 | 77.556 | 56.158 | 1.00 | 25.57 |
| ATOM | 11109 | CA | GLU | B | 660 | 66.691 | 78.180 | 55.733 | 1.00 | 26.05 |
| ATOM | 11110 | C | GLU | B | 660 | 65.523 | 77.225 | 55.997 | 1.00 | 25.19 |
| ATOM | 11111 | O | GLU | B | 660 | 64.406 | 77.669 | 56.099 | 1.00 | 25.53 |
| ATOM | 11112 | CB | GLU | B | 660 | 66.702 | 78.651 | 54.243 | 1.00 | 27.16 |
| ATOM | 11113 | CG | GLU | B | 660 | 67.219 | 80.087 | 54.084 | 1.00 | 31.79 |
| ATOM | 11114 | CD | GLU | B | 660 | 67.825 | 80.417 | 52.703 | 1.00 | 35.43 |
| ATOM | 11115 | OE1 | GLU | B | 660 | 68.987 | 80.034 | 52.429 | 1.00 | 36.77 |
| ATOM | 11116 | OE2 | GLU | B | 660 | 67.151 | 81.092 | 51.884 | 1.00 | 38.64 |
| ATOM | 11117 | N | TYR | B | 661 | 65.745 | 75.931 | 56.172 | 1.00 | 24.04 |
| ATOM | 11118 | CA | TYR | B | 661 | 64.596 | 75.073 | 56.481 | 1.00 | 23.76 |
| ATOM | 11119 | C | TYR | B | 661 | 64.227 | 75.006 | 57.960 | 1.00 | 23.19 |
| ATOM | 11120 | O | TYR | B | 661 | 63.156 | 74.455 | 58.326 | 1.00 | 23.04 |
| ATOM | 11121 | CB | TYR | B | 661 | 64.844 | 73.629 | 56.023 | 1.00 | 24.22 |
| ATOM | 11122 | CG | TYR | B | 661 | 65.054 | 73.451 | 54.570 | 1.00 | 23.41 |
| ATOM | 11123 | CD1 | TYR | B | 661 | 64.499 | 74.306 | 53.638 | 1.00 | 25.96 |
| ATOM | 11124 | CD2 | TYR | B | 661 | 65.813 | 72.391 | 54.102 | 1.00 | 28.26 |
| ATOM | 11125 | CE1 | TYR | B | 661 | 64.750 | 74.139 | 52.255 | 1.00 | 24.48 |
| ATOM | 11126 | CE2 | TYR | B | 661 | 66.026 | 72.195 | 52.733 | 1.00 | 26.73 |
| ATOM | 11127 | CZ | TYR | B | 661 | 65.500 | 73.078 | 51.826 | 1.00 | 27.30 |
| ATOM | 11128 | OH | TYR | B | 661 | 65.727 | 72.842 | 50.473 | 1.00 | 31.51 |
| ATOM | 11129 | N | TYR | B | 662 | 65.093 | 75.552 | 58.817 | 1.00 | 22.68 |
| ATOM | 11130 | CA | TYR | B | 662 | 64.879 | 75.452 | 60.256 | 1.00 | 22.16 |
| ATOM | 11131 | C | TYR | B | 662 | 64.161 | 76.652 | 60.890 | 1.00 | 22.58 |
| ATOM | 11132 | O | TYR | B | 662 | 63.988 | 77.668 | 60.239 | 1.00 | 21.76 |
| ATOM | 11133 | CB | TYR | B | 662 | 66.164 | 75.072 | 60.980 | 1.00 | 22.02 |
| ATOM | 11134 | CG | TYR | B | 662 | 65.868 | 74.449 | 62.325 | 1.00 | 21.48 |
| ATOM | 11135 | CD1 | TYR | B | 662 | 65.003 | 73.388 | 62.411 | 1.00 | 21.91 |
| ATOM | 11136 | CD2 | TYR | B | 662 | 66.387 | 74.967 | 63.495 | 1.00 | 23.40 |
| ATOM | 11137 | CE1 | TYR | B | 662 | 64.649 | 72.838 | 63.607 | 1.00 | 22.92 |
| ATOM | 11138 | CE2 | TYR | B | 662 | 66.054 | 74.383 | 64.770 | 1.00 | 24.30 |
| ATOM | 11139 | CZ | TYR | B | 662 | 65.174 | 73.322 | 64.797 | 1.00 | 24.12 |
| ATOM | 11140 | OH | TYR | B | 662 | 64.801 | 72.662 | 65.959 | 1.00 | 19.23 |
| ATOM | 11141 | N | ASP | B | 663 | 63.669 | 76.493 | 62.123 | 1.00 | 22.74 |
| ATOM | 11142 | CA | ASP | B | 663 | 62.882 | 77.560 | 62.761 | 1.00 | 24.01 |
| ATOM | 11143 | C | ASP | B | 663 | 63.660 | 78.860 | 63.030 | 1.00 | 24.75 |
| ATOM | 11144 | O | ASP | B | 663 | 64.884 | 78.873 | 63.182 | 1.00 | 24.67 |
| ATOM | 11145 | CB | ASP | B | 663 | 62.075 | 77.087 | 63.970 | 1.00 | 23.36 |
| ATOM | 11146 | CG | ASP | B | 663 | 62.895 | 76.808 | 65.212 | 1.00 | 24.97 |
| ATOM | 11147 | OD1 | ASP | B | 663 | 63.512 | 77.721 | 65.839 | 1.00 | 21.98 |
| ATOM | 11148 | OD2 | ASP | B | 663 | 62.889 | 75.668 | 65.707 | 1.00 | 27.41 |
| ATOM | 11149 | N | SER | B | 664 | 62.919 | 79.956 | 63.000 | 1.00 | 25.55 |
| ATOM | 11150 | CA | SER | B | 664 | 63.519 | 81.272 | 63.135 | 1.00 | 26.43 |
| ATOM | 11151 | C | SER | B | 664 | 64.241 | 81.495 | 64.431 | 1.00 | 26.07 |
| ATOM | 11152 | O | SER | B | 664 | 65.350 | 81.982 | 64.430 | 1.00 | 26.65 |
| ATOM | 11153 | CB | SER | B | 664 | 62.452 | 82.368 | 62.995 | 1.00 | 26.25 |
| ATOM | 11154 | OG | SER | B | 664 | 61.415 | 82.164 | 63.911 | 1.00 | 25.95 |
| ATOM | 11155 | N | VAL | B | 665 | 63.599 | 81.167 | 65.543 | 1.00 | 25.54 |
| ATOM | 11156 | CA | VAL | B | 665 | 64.180 | 81.466 | 66.822 | 1.00 | 25.02 |
| ATOM | 11157 | C | VAL | B | 665 | 65.531 | 80.820 | 67.018 | 1.00 | 25.27 |
| ATOM | 11158 | O | VAL | B | 665 | 66.462 | 81.471 | 67.493 | 1.00 | 25.08 |
| ATOM | 11159 | CB | VAL | B | 665 | 63.250 | 81.065 | 68.007 | 1.00 | 25.18 |
| ATOM | 11160 | CG1 | VAL | B | 665 | 63.897 | 81.417 | 69.407 | 1.00 | 24.49 |
| ATOM | 11161 | CG2 | VAL | B | 665 | 61.913 | 81.772 | 67.909 | 1.00 | 24.54 |
| ATOM | 11162 | N | TYR | B | 666 | 65.640 | 79.528 | 66.709 | 1.00 | 24.73 |
| ATOM | 11163 | CA | TYR | B | 666 | 66.872 | 78.828 | 66.941 | 1.00 | 23.57 |
| ATOM | 11164 | C | TYR | B | 666 | 67.916 | 79.252 | 65.928 | 1.00 | 24.38 |
| ATOM | 11165 | O | TYR | B | 666 | 69.052 | 79.527 | 66.308 | 1.00 | 24.18 |
| ATOM | 11166 | CB | TYR | B | 666 | 66.697 | 77.316 | 66.900 | 1.00 | 23.24 |
| ATOM | 11167 | CG | TYR | B | 666 | 67.994 | 76.469 | 67.056 | 1.00 | 21.71 |
| ATOM | 11168 | CD1 | TYR | B | 666 | 68.850 | 76.288 | 65.993 | 1.00 | 22.85 |
| ATOM | 11169 | CD2 | TYR | B | 666 | 68.300 | 75.864 | 68.254 | 1.00 | 25.29 |
| ATOM | 11170 | CE1 | TYR | B | 666 | 69.985 | 75.538 | 66.098 | 1.00 | 25.06 |
| ATOM | 11171 | CE2 | TYR | B | 666 | 69.470 | 75.074 | 68.414 | 1.00 | 27.49 |
| ATOM | 11172 | CZ | TYR | B | 666 | 70.306 | 74.934 | 67.322 | 1.00 | 27.05 |
| ATOM | 11173 | OH | TYR | B | 666 | 71.421 | 74.186 | 67.419 | 1.00 | 27.36 |
| ATOM | 11174 | N | THR | B | 667 | 67.532 | 79.343 | 64.658 | 1.00 | 24.04 |
| ATOM | 11175 | CA | THR | B | 667 | 68.504 | 79.562 | 63.599 | 1.00 | 24.34 |
| ATOM | 11176 | C | THR | B | 667 | 69.097 | 80.944 | 63.584 | 1.00 | 25.40 |
| ATOM | 11177 | O | THR | B | 667 | 70.315 | 81.104 | 63.493 | 1.00 | 24.23 |
| ATOM | 11178 | CB | THR | B | 667 | 67.848 | 79.313 | 62.258 | 1.00 | 25.25 |
| ATOM | 11179 | OG1 | THR | B | 667 | 67.267 | 78.003 | 62.248 | 1.00 | 25.15 |
| ATOM | 11180 | CG2 | THR | B | 667 | 68.876 | 79.340 | 61.107 | 1.00 | 23.81 |
| ATOM | 11181 | N | GLU | B | 668 | 68.217 | 81.940 | 63.678 | 1.00 | 26.00 |
| ATOM | 11182 | CA | GLU | B | 668 | 68.639 | 83.324 | 63.599 | 1.00 | 26.42 |
| ATOM | 11183 | C | GLU | B | 668 | 69.486 | 83.675 | 64.817 | 1.00 | 27.19 |
| ATOM | 11184 | O | GLU | B | 668 | 70.377 | 84.522 | 64.740 | 1.00 | 27.51 |
| ATOM | 11185 | CB | GLU | B | 668 | 67.417 | 84.233 | 63.498 | 1.00 | 25.94 |
| ATOM | 11186 | CG | GLU | B | 668 | 66.572 | 83.896 | 62.243 | 1.00 | 27.24 |
| ATOM | 11187 | CD | GLU | B | 668 | 65.231 | 84.553 | 62.236 | 1.00 | 28.69 |
| ATOM | 11188 | OE1 | GLU | B | 668 | 65.018 | 85.479 | 63.066 | 1.00 | 29.27 |
| ATOM | 11189 | OE2 | GLU | B | 668 | 64.380 | 84.162 | 61.394 | 1.00 | 30.62 |
| ATOM | 11190 | N | ARG | B | 669 | 69.252 | 82.995 | 65.928 | 1.00 | 27.00 |
| ATOM | 11191 | CA | ARG | B | 669 | 70.044 | 83.287 | 67.101 | 1.00 | 27.41 |
| ATOM | 11192 | C | ARG | B | 669 | 71.528 | 83.126 | 66.810 | 1.00 | 27.58 |
| ATOM | 11193 | O | ARG | B | 669 | 72.339 | 83.880 | 67.335 | 1.00 | 27.79 |
| ATOM | 11194 | CB | ARG | B | 669 | 69.679 | 82.386 | 68.276 | 1.00 | 26.74 |
| ATOM | 11195 | CG | ARG | B | 669 | 70.552 | 82.687 | 69.524 | 1.00 | 28.52 |
| ATOM | 11196 | CD | ARG | B | 669 | 69.894 | 82.262 | 70.799 | 1.00 | 30.22 |
| ATOM | 11197 | NE | ARG | B | 669 | 69.815 | 80.820 | 70.853 | 1.00 | 32.37 |
| ATOM | 11198 | CZ | ARG | B | 669 | 68.708 | 80.079 | 70.812 | 1.00 | 31.32 |
| ATOM | 11199 | NH1 | ARG | B | 669 | 67.505 | 80.610 | 70.736 | 1.00 | 32.60 |
| ATOM | 11200 | NH2 | ARG | B | 669 | 68.835 | 78.762 | 70.875 | 1.00 | 32.35 |
| ATOM | 11201 | N | TYR | B | 670 | 71.877 | 82.106 | 66.031 | 1.00 | 28.01 |
| ATOM | 11202 | CA | TYR | B | 670 | 73.256 | 81.851 | 65.678 | 1.00 | 28.42 |
| ATOM | 11203 | C | TYR | B | 670 | 73.632 | 82.365 | 64.286 | 1.00 | 29.28 |
| ATOM | 11204 | O | TYR | B | 670 | 74.809 | 82.531 | 63.980 | 1.00 | 28.53 |
| ATOM | 11205 | CB | TYR | B | 670 | 73.569 | 80.351 | 65.733 | 1.00 | 28.12 |
| ATOM | 11206 | CG | TYR | B | 670 | 73.046 | 79.710 | 66.989 | 1.00 | 28.98 |
| ATOM | 11207 | CD1 | TYR | B | 670 | 73.635 | 79.983 | 68.210 | 1.00 | 31.34 |
| ATOM | 11208 | CD2 | TYR | B | 670 | 71.918 | 78.904 | 66.971 | 1.00 | 27.11 |
| ATOM | 11209 | CE1 | TYR | B | 670 | 73.130 | 79.450 | 69.394 | 1.00 | 29.84 |
| ATOM | 11210 | CE2 | TYR | B | 670 | 71.416 | 78.344 | 68.151 | 1.00 | 26.97 |
| ATOM | 11211 | CZ | TYR | B | 670 | 72.029 | 78.622 | 69.341 | 1.00 | 27.33 |
| ATOM | 11212 | OH | TYR | B | 670 | 71.533 | 78.119 | 70.512 | 1.00 | 28.94 |
| ATOM | 11213 | N | MET | B | 671 | 72.667 | 82.625 | 63.431 | 1.00 | 30.33 |
| ATOM | 11214 | CA | MET | B | 671 | 73.046 | 82.882 | 62.049 | 1.00 | 30.67 |
| ATOM | 11215 | C | MET | B | 671 | 72.600 | 84.235 | 61.536 | 1.00 | 31.06 |
| ATOM | 11216 | O | MET | B | 671 | 72.901 | 84.586 | 60.392 | 1.00 | 30.69 |
| ATOM | 11217 | CB | MET | B | 671 | 72.442 | 81.795 | 61.132 | 1.00 | 30.61 |
| ATOM | 11218 | CG | MET | B | 672 | 73.115 | 80.414 | 61.185 | 1.00 | 30.65 |
| ATOM | 11219 | SD | MET | B | 671 | 74.640 | 80.253 | 60.254 | 1.00 | 30.11 |
| ATOM | 11220 | CE | MET | B | 671 | 73.905 | 80.430 | 58.601 | 1.00 | 32.95 |
| ATOM | 11221 | N | GLY | B | 672 | 71.831 | 84.952 | 62.336 | 1.00 | 31.71 |
| ATOM | 11222 | CA | GLY | B | 672 | 71.235 | 86.197 | 61.891 | 1.00 | 32.29 |
| ATOM | 11223 | C | GLY | B | 672 | 70.160 | 85.871 | 60.860 | 1.00 | 32.93 |
| ATOM | 11224 | O | GLY | B | 672 | 69.617 | 84.779 | 60.870 | 1.00 | 32.80 |
| ATOM | 11225 | N | LEU | B | 673 | 69.854 | 86.820 | 59.983 | 1.00 | 33.57 |
| ATOM | 11226 | CA | LEU | B | 673 | 68.852 | 86.643 | 58.947 | 1.00 | 34.26 |
| ATOM | 11227 | C | LEU | B | 673 | 69.450 | 86.222 | 57.621 | 1.00 | 35.15 |
| ATOM | 11228 | O | LEU | B | 673 | 70.515 | 86.694 | 57.218 | 1.00 | 33.95 |
| ATOM | 11229 | CB | LEU | B | 673 | 68.112 | 87.956 | 58.741 | 1.00 | 34.73 |
| ATOM | 11230 | CG | LEU | B | 673 | 67.448 | 88.500 | 60.011 | 1.00 | 36.45 |
| ATOM | 11231 | CD1 | LEU | B | 673 | 66.879 | 89.870 | 59.764 | 1.00 | 36.42 |
| ATOM | 11232 | CD2 | LEU | B | 673 | 66.362 | 87.532 | 60.417 | 1.00 | 36.99 |
| ATOM | 11233 | N | PRO | B | 674 | 68.749 | 85.352 | 56.903 | 1.00 | 37.00 |
| ATOM | 11234 | CA | PRO | B | 674 | 69.217 | 84.926 | 55.600 | 1.00 | 38.48 |
| ATOM | 11235 | C | PRO | B | 674 | 68.829 | 85.963 | 54.528 | 1.00 | 40.15 |
| ATOM | 11236 | O | PRO | B | 674 | 68.021 | 85.646 | 53.645 | 1.00 | 39.73 |
| ATOM | 11237 | CB | PRO | B | 674 | 68.464 | 83.635 | 55.425 | 1.00 | 37.86 |
| ATOM | 11238 | CG | PRO | B | 674 | 67.139 | 84.010 | 55.931 | 1.00 | 38.01 |
| ATOM | 11239 | CD | PRO | B | 674 | 67.477 | 84.689 | 57.226 | 1.00 | 37.42 |
| ATOM | 11240 | N | THR | B | 675 | 69.366 | 87.182 | 54.646 | 1.00 | 41.47 |
| ATOM | 11241 | CA | THR | B | 675 | 69.218 | 88.225 | 53.623 | 1.00 | 43.02 |
| ATOM | 11242 | C | THR | B | 675 | 70.611 | 88.638 | 53.146 | 1.00 | 43.72 |
| ATOM | 11243 | O | THR | B | 675 | 71.604 | 88.445 | 53.856 | 1.00 | 43.16 |
| ATOM | 11244 | CB | THR | B | 675 | 68.479 | 89.477 | 54.164 | 1.00 | 43.15 |
| ATOM | 11245 | OG1 | THR | B | 675 | 69.224 | 90.066 | 55.243 | 1.00 | 45.83 |
| ATOM | 11246 | CG2 | THR | B | 675 | 67.126 | 89.136 | 54.787 | 1.00 | 43.20 |
| ATOM | 11247 | N | PRO | B | 676 | 70.700 | 89.191 | 51.937 | 1.00 | 44.24 |
| ATOM | 11248 | CA | PRO | B | 676 | 71.991 | 89.634 | 51.380 | 1.00 | 44.30 |
| ATOM | 11249 | C | PRO | B | 676 | 72.603 | 90.713 | 52.256 | 1.00 | 43.57 |
| ATOM | 11250 | O | PRO | B | 676 | 73.800 | 90.838 | 52.439 | 1.00 | 43.64 |
| ATOM | 11251 | CB | PRO | B | 676 | 71.591 | 90.231 | 50.014 | 1.00 | 44.31 |
| ATOM | 11252 | CG | PRO | B | 676 | 70.307 | 89.548 | 49.685 | 1.00 | 44.53 |
| ATOM | 11253 | CD | PRO | B | 676 | 69.588 | 89.428 | 51.000 | 1.00 | 44.58 |
| ATOM | 11254 | N | GLU | B | 677 | 71.701 | 91.491 | 52.798 | 1.00 | 43.67 |
| ATOM | 11255 | CA | GLU | B | 677 | 71.973 | 92.572 | 53.706 | 1.00 | 44.03 |
| ATOM | 11256 | C | GLU | B | 677 | 72.621 | 92.019 | 54.986 | 1.00 | 43.44 |
| ATOM | 11257 | O | GLU | B | 677 | 73.256 | 92.771 | 55.728 | 1.00 | 43.51 |
| ATOM | 11258 | CB | GLU | B | 677 | 70.625 | 93.245 | 54.036 | 1.00 | 44.76 |
| ATOM | 11259 | CG | GLU | B | 677 | 69.575 | 93.052 | 52.916 | 1.00 | 47.36 |
| ATOM | 11260 | CD | GLU | B | 677 | 68.135 | 93.125 | 53.392 | 1.00 | 51.20 |
| ATOM | 11261 | OE1 | GLU | B | 677 | 67.866 | 93.836 | 54.381 | 1.00 | 54.53 |
| ATOM | 11262 | OE2 | GLU | B | 677 | 67.256 | 92.479 | 52.770 | 1.00 | 54.13 |
| ATOM | 11263 | N | ASP | B | 678 | 72.482 | 90.720 | 55.251 | 1.00 | 41.72 |
| ATOM | 11264 | CA | ASP | B | 678 | 73.013 | 90.185 | 56.502 | 1.00 | 40.99 |
| ATOM | 11265 | C | ASP | B | 678 | 73.846 | 88.917 | 56.341 | 1.00 | 39.07 |
| ATOM | 11266 | O | ASP | B | 678 | 75.044 | 88.986 | 56.074 | 1.00 | 37.79 |
| ATOM | 11267 | CB | ASP | B | 678 | 71.882 | 89.992 | 57.538 | 1.00 | 41.34 |
| ATOM | 11268 | CG | ASP | B | 678 | 72.413 | 89.618 | 58.936 | 1.00 | 42.84 |
| ATOM | 11269 | OD1 | ASP | B | 678 | 73.628 | 89.707 | 59.162 | 1.00 | 46.59 |
| ATOM | 11270 | OD2 | ASP | B | 678 | 71.699 | 89.210 | 59.868 | 1.00 | 45.40 |
| ATOM | 11271 | N | ASN | B | 679 | 73.240 | 87.751 | 56.491 | 1.00 | 37.03 |
| ATOM | 11272 | CA | ASN | B | 679 | 74.061 | 86.561 | 56.470 | 1.00 | 36.29 |
| ATOM | 11273 | C | ASN | B | 679 | 73.701 | 85.502 | 55.435 | 1.00 | 35.66 |
| ATOM | 11274 | O | ASN | B | 679 | 74.024 | 84.344 | 55.623 | 1.00 | 35.59 |
| ATOM | 11275 | CB | ASN | B | 679 | 74.112 | 85.966 | 57.888 | 1.00 | 35.38 |
| ATOM | 11276 | CG | ASN | B | 679 | 75.315 | 85.076 | 58.093 | 1.00 | 35.81 |
| ATOM | 11277 | OD1 | ASN | B | 679 | 75.271 | 84.065 | 58.829 | 1.00 | 36.44 |
| ATOM | 11278 | ND2 | ASN | B | 679 | 76.407 | 85.429 | 57.426 | 1.00 | 30.49 |
| ATOM | 11279 | N | LEU | B | 680 | 73.074 | 85.893 | 54.330 | 1.00 | 36.18 |
| ATOM | 11280 | CA | LEU | B | 680 | 72.665 | 84.940 | 53.282 | 1.00 | 37.04 |
| ATOM | 11281 | C | LEU | B | 680 | 73.759 | 84.052 | 52.745 | 1.00 | 37.17 |
| ATOM | 11282 | O | LEU | B | 680 | 73.558 | 82.845 | 52.559 | 1.00 | 37.42 |
| ATOM | 11283 | CB | LEU | B | 680 | 72.076 | 85.664 | 52.075 | 1.00 | 37.58 |
| ATOM | 11284 | CG | LEU | B | 680 | 70.958 | 85.022 | 51.240 | 1.00 | 39.69 |
| ATOM | 11285 | CD1 | LEU | B | 680 | 71.226 | 85.257 | 49.772 | 1.00 | 41.11 |
| ATOM | 11286 | CD2 | LEU | B | 680 | 70.729 | 83.532 | 51.484 | 1.00 | 40.41 |
| ATOM | 11287 | N | ASP | B | 681 | 74.926 | 84.623 | 52.483 | 1.00 | 37.50 |
| ATOM | 11288 | CA | ASP | B | 681 | 75.964 | 83.855 | 51.830 | 1.00 | 38.01 |
| ATOM | 11289 | C | ASP | B | 681 | 76.345 | 82.586 | 52.632 | 1.00 | 37.14 |
| ATOM | 11290 | O | ASP | B | 681 | 76.483 | 81.483 | 52.065 | 1.00 | 36.38 |
| ATOM | 11291 | CB | ASP | B | 681 | 77.199 | 84.722 | 51.515 | 1.00 | 39.14 |
| ATOM | 11292 | CG | ASP | B | 681 | 76.887 | 85.920 | 50.571 | 1.00 | 43.26 |
| ATOM | 11293 | OD1 | ASP | B | 681 | 76.109 | 85.762 | 49.609 | 1.00 | 46.71 |
| ATOM | 11294 | OD2 | ASP | B | 681 | 77.384 | 87.070 | 50.723 | 1.00 | 48.74 |
| ATOM | 11295 | N | HIS | B | 682 | 76.568 | 82.742 | 53.929 | 1.00 | 35.59 |
| ATOM | 11296 | CA | HIS | B | 682 | 76.893 | 81.582 | 54.738 | 1.00 | 35.67 |
| ATOM | 11297 | C | HIS | B | 682 | 75.673 | 80.632 | 54.930 | 1.00 | 33.61 |
| ATOM | 11298 | O | HIS | B | 682 | 75.867 | 79.452 | 55.143 | 1.00 | 33.15 |
| ATOM | 11299 | CB | HIS | B | 682 | 77.524 | 81.927 | 56.099 | 1.00 | 35.65 |
| ATOM | 11300 | CG | HIS | B | 682 | 78.040 | 80.714 | 56.806 | 1.00 | 37.66 |
| ATOM | 11301 | ND1 | HIS | B | 682 | 77.298 | 80.030 | 57.750 | 1.00 | 37.90 |
| ATOM | 11302 | CD2 | HIS | B | 682 | 79.190 | 80.012 | 56.659 | 1.00 | 38.17 |
| ATOM | 11303 | CE1 | HIS | B | 682 | 77.969 | 78.967 | 58.159 | 1.00 | 31.97 |
| ATOM | 11304 | NE2 | HIS | B | 682 | 79.123 | 78.935 | 57.518 | 1.00 | 37.82 |
| ATOM | 11305 | N | TYR | B | 683 | 74.448 | 81.149 | 54.879 | 1.00 | 32.10 |
| ATOM | 11306 | CA | TYR | B | 683 | 73.285 | 80.257 | 54.857 | 1.00 | 31.92 |
| ATOM | 11307 | C | TYR | B | 683 | 73.414 | 79.342 | 53.630 | 1.00 | 31.93 |
| ATOM | 11308 | O | TYR | B | 683 | 73.244 | 78.158 | 53.724 | 1.00 | 28.90 |
| ATOM | 11309 | CB | TYR | B | 683 | 71.986 | 81.044 | 54.770 | 1.00 | 31.15 |
| ATOM | 11310 | CG | TYR | B | 683 | 71.275 | 81.405 | 56.071 | 1.00 | 30.21 |
| ATOM | 11311 | CD1 | TYR | B | 683 | 71.535 | 82.598 | 56.716 | 1.00 | 29.38 |
| ATOM | 11312 | CD2 | TYR | B | 683 | 70.290 | 80.571 | 56.619 | 1.00 | 28.21 |
| ATOM | 11313 | CE1 | TYR | B | 683 | 70.866 | 82.965 | 57.828 | 1.00 | 27.01 |
| ATOM | 11314 | CE2 | TYR | B | 683 | 69.611 | 80.935 | 57.728 | 1.00 | 27.18 |
| ATOM | 11315 | CZ | TYR | B | 683 | 69.905 | 82.136 | 58.339 | 1.00 | 27.90 |
| ATOM | 11316 | OH | TYR | B | 683 | 69.254 | 82.523 | 59.472 | 1.00 | 27.60 |
| ATOM | 11317 | N | ARG | B | 684 | 73.825 | 79.909 | 52.490 | 1.00 | 33.70 |
| ATOM | 11318 | CA | ARG | B | 684 | 73.958 | 79.138 | 51.231 | 1.00 | 33.97 |
| ATOM | 11319 | C | ARG | B | 684 | 75.188 | 78.258 | 51.116 | 1.00 | 33.32 |
| ATOM | 11320 | O | ARG | B | 684 | 75.154 | 77.247 | 50.441 | 1.00 | 31.95 |
| ATOM | 11321 | CB | ARG | B | 684 | 73.975 | 80.080 | 50.031 | 1.00 | 35.11 |
| ATOM | 11322 | CG | ARG | B | 684 | 72.642 | 80.274 | 49.422 | 1.00 | 39.47 |
| ATOM | 11323 | CD | ARG | B | 684 | 71.744 | 80.970 | 50.302 | 1.00 | 42.04 |
| ATOM | 11324 | NE | ARG | B | 684 | 70.290 | 80.772 | 50.137 | 1.00 | 45.44 |
| ATOM | 11325 | CZ | ARG | B | 684 | 69.535 | 81.284 | 49.182 | 1.00 | 45.72 |
| ATOM | 11326 | NH1 | ARG | B | 684 | 70.057 | 81.935 | 48.153 | 1.00 | 46.39 |
| ATOM | 11327 | NH2 | ARG | B | 684 | 68.238 | 81.139 | 49.267 | 1.00 | 46.70 |
| ATOM | 11328 | N | ASN | B | 685 | 76.275 | 78.662 | 51.766 | 1.00 | 32.64 |
| ATOM | 11329 | CA | ASN | B | 685 | 77.527 | 77.935 | 51.742 | 1.00 | 32.24 |
| ATOM | 11330 | C | ASN | B | 685 | 77.526 | 76.786 | 52.737 | 1.00 | 31.28 |
| ATOM | 11331 | O | ASN | B | 685 | 78.473 | 76.008 | 52.780 | 1.00 | 30.38 |
| ATOM | 11332 | CB | ASN | B | 685 | 78.681 | 78.900 | 52.170 | 1.00 | 33.42 |
| ATOM | 11333 | CG | ASN | B | 685 | 79.238 | 79.730 | 51.006 | 1.00 | 38.29 |
| ATOM | 11334 | OD1 | ASN | B | 685 | 78.759 | 79.629 | 49.844 | 1.00 | 44.79 |
| ATOM | 11335 | ND2 | ASN | B | 685 | 80.270 | 80.551 | 51.293 | 1.00 | 42.71 |
| ATOM | 11336 | N | SER | B | 686 | 76.493 | 76.679 | 53.579 | 1.00 | 29.78 |
| ATOM | 11337 | CA | SER | B | 686 | 76.580 | 75.721 | 54.678 | 1.00 | 28.50 |
| ATOM | 11338 | C | SER | B | 686 | 75.479 | 74.671 | 54.712 | 1.00 | 28.26 |
| ATOM | 11339 | O | SER | B | 686 | 75.116 | 74.186 | 55.785 | 1.00 | 26.91 |
| ATOM | 11340 | CB | SER | B | 686 | 76.587 | 76.461 | 56.009 | 1.00 | 27.40 |
| ATOM | 11341 | OG | SER | B | 686 | 75.380 | 77.183 | 56.153 | 1.00 | 27.07 |
| ATOM | 11342 | N | THR | B | 687 | 75.014 | 74.283 | 53.532 | 1.00 | 28.34 |
| ATOM | 11343 | CA | THR | B | 687 | 74.061 | 73.221 | 53.366 | 1.00 | 27.78 |
| ATOM | 11344 | C | THR | B | 687 | 74.820 | 71.912 | 53.120 | 1.00 | 28.66 |
| ATOM | 11345 | O | THR | B | 687 | 75.969 | 71.921 | 52.657 | 1.00 | 28.67 |
| ATOM | 11346 | CB | THR | B | 687 | 73.203 | 73.452 | 52.142 | 1.00 | 27.60 |
| ATOM | 11347 | OG1 | THR | B | 687 | 74.026 | 73.394 | 50.971 | 1.00 | 30.02 |
| ATOM | 11348 | CG2 | THR | B | 687 | 72.548 | 74.850 | 52.110 | 1.00 | 28.26 |
| ATOM | 11349 | N | VAL | B | 688 | 74.159 | 70.791 | 53.420 | 1.00 | 27.61 |
| ATOM | 11350 | CA | VAL | B | 688 | 74.678 | 69.485 | 53.139 | 1.00 | 27.53 |
| ATOM | 11351 | C | VAL | B | 688 | 74.680 | 69.231 | 51.619 | 1.00 | 27.76 |
| ATOM | 11352 | O | VAL | B | 688 | 75.573 | 68.569 | 51.086 | 1.00 | 25.95 |
| ATOM | 11353 | CB | VAL | B | 688 | 73.801 | 68.405 | 53.828 | 1.00 | 27.65 |
| ATOM | 11354 | CG1 | VAL | B | 688 | 74.189 | 67.016 | 53.405 | 1.00 | 27.71 |
| ATOM | 11355 | CG2 | VAL | B | 688 | 73.874 | 68.538 | 55.383 | 1.00 | 28.56 |
| ATOM | 11356 | N | MET | B | 689 | 73.655 | 69.732 | 50.938 | 1.00 | 28.69 |
| ATOM | 11357 | CA | MET | B | 689 | 73.477 | 69.496 | 49.500 | 1.00 | 29.91 |
| ATOM | 11358 | C | MET | B | 689 | 74.662 | 69.928 | 48.657 | 1.00 | 30.86 |
| ATOM | 11359 | O | MET | B | 689 | 75.003 | 69.255 | 47.721 | 1.00 | 31.07 |
| ATOM | 11360 | CB | MET | B | 689 | 72.234 | 70.231 | 48.981 | 1.00 | 29.60 |
| ATOM | 11361 | CG | MET | B | 689 | 70.930 | 69.589 | 49.381 | 1.00 | 30.81 |
| ATOM | 11362 | SD | MET | B | 689 | 70.459 | 69.694 | 51.143 | 1.00 | 28.72 |
| ATOM | 11363 | CE | MET | B | 689 | 69.884 | 71.281 | 51.212 | 1.00 | 29.52 |
| ATOM | 11364 | N | SER | B | 690 | 75.285 | 71.043 | 49.014 | 1.00 | 32.36 |
| ATOM | 11365 | CA | SER | B | 690 | 76.424 | 71.574 | 48.286 | 1.00 | 33.32 |
| ATOM | 11366 | C | SER | B | 690 | 77.614 | 70.628 | 48.314 | 1.00 | 33.16 |
| ATOM | 11367 | O | SER | B | 690 | 78.499 | 70.717 | 47.474 | 1.00 | 33.15 |
| ATOM | 11368 | CB | SER | B | 690 | 76.845 | 72.948 | 48.858 | 1.00 | 34.19 |
| ATOM | 11369 | OG | SER | B | 690 | 77.345 | 72.867 | 50.191 | 1.00 | 34.77 |
| ATOM | 11370 | N | ARG | B | 691 | 77.628 | 69.709 | 49.261 | 1.00 | 32.36 |
| ATOM | 11371 | CA | ARG | B | 691 | 78.734 | 68.783 | 49.374 | 1.00 | 31.79 |
| ATOM | 11372 | C | ARG | B | 691 | 78.420 | 67.428 | 48.792 | 1.00 | 31.37 |
| ATOM | 11373 | O | ARG | B | 691 | 79.177 | 66.516 | 48.970 | 1.00 | 31.21 |
| ATOM | 11374 | CB | ARG | B | 691 | 79.083 | 68.599 | 50.840 | 1.00 | 32.03 |
| ATOM | 11375 | CG | ARG | B | 691 | 79.171 | 69.886 | 51.582 | 1.00 | 33.93 |
| ATOM | 11376 | CD | ARG | B | 691 | 79.839 | 69.788 | 52.943 | 1.00 | 34.80 |
| ATOM | 11377 | NE | ARG | B | 691 | 80.389 | 71.079 | 53.379 | 1.00 | 35.85 |
| ATOM | 11378 | CZ | ARG | B | 691 | 81.344 | 71.205 | 54.309 | 1.00 | 34.51 |
| ATOM | 11379 | NH1 | ARG | B | 691 | 81.836 | 70.137 | 54.906 | 1.00 | 30.90 |
| ATOM | 11380 | NH2 | ARG | B | 691 | 81.789 | 72.405 | 54.651 | 1.00 | 36.61 |
| ATOM | 11381 | N | ALA | B | 692 | 77.319 | 67.305 | 48.064 | 1.00 | 31.22 |
| ATOM | 11382 | CA | ALA | B | 692 | 76.877 | 66.016 | 47.558 | 1.00 | 31.18 |
| ATOM | 11383 | C | ALA | B | 692 | 77.953 | 65.161 | 46.871 | 1.00 | 31.15 |
| ATOM | 11384 | O | ALA | B | 692 | 78.087 | 63.959 | 47.160 | 1.00 | 30.04 |
| ATOM | 11385 | CB | ALA | B | 692 | 75.692 | 66.245 | 46.604 | 1.00 | 31.81 |
| ATOM | 11386 | N | GLU | B | 693 | 78.727 | 65.767 | 45.980 | 1.00 | 31.68 |
| ATOM | 11387 | CA | GLU | B | 693 | 79.738 | 65.032 | 45.223 | 1.00 | 32.97 |
| ATOM | 11388 | C | GLU | B | 693 | 80.723 | 64.204 | 46.083 | 1.00 | 32.82 |
| ATOM | 11389 | O | GLU | B | 693 | 81.062 | 63.089 | 45.698 | 1.00 | 31.25 |
| ATOM | 11390 | CB | GLU | B | 693 | 80.494 | 65.971 | 44.273 | 1.00 | 34.43 |
| ATOM | 11391 | CG | GLU | B | 693 | 79.567 | 67.035 | 43.690 | 1.00 | 40.37 |
| ATOM | 11392 | CD | GLU | B | 693 | 79.988 | 67.617 | 42.355 | 1.00 | 49.09 |
| ATOM | 11393 | OE1 | GLU | B | 693 | 80.533 | 66.861 | 41.489 | 1.00 | 54.53 |
| ATOM | 11394 | OE2 | GLU | B | 693 | 79.732 | 68.843 | 42.166 | 1.00 | 53.36 |
| ATOM | 11395 | N | ASN | B | 694 | 81.134 | 64.715 | 47.248 | 1.00 | 33.08 |
| ATOM | 11396 | CA | ASN | B | 694 | 82.109 | 64.029 | 48.119 | 1.00 | 33.68 |
| ATOM | 11397 | C | ASN | B | 694 | 81.570 | 62.806 | 48.868 | 1.00 | 34.46 |
| ATOM | 11398 | O | ASN | B | 694 | 82.305 | 62.082 | 49.544 | 1.00 | 35.44 |
| ATOM | 11399 | CB | ASN | B | 694 | 82.682 | 65.018 | 49.143 | 1.00 | 34.38 |
| ATOM | 11400 | CG | ASN | B | 694 | 83.586 | 66.086 | 48.505 | 1.00 | 35.36 |
| ATOM | 11401 | OD1 | ASN | B | 694 | 84.453 | 65.786 | 47.686 | 1.00 | 40.24 |
| ATOM | 11402 | ND2 | ASN | B | 694 | 83.386 | 67.316 | 48.888 | 1.00 | 34.81 |
| ATOM | 11403 | N | PHE | B | 695 | 80.269 | 62.571 | 48.777 | 1.00 | 34.92 |
| ATOM | 11404 | CA | PHE | B | 695 | 79.689 | 61.450 | 49.453 | 1.00 | 34.22 |
| ATOM | 11405 | C | PHE | B | 695 | 79.991 | 60.193 | 48.692 | 1.00 | 34.61 |
| ATOM | 11406 | O | PHE | B | 695 | 79.716 | 59.100 | 49.181 | 1.00 | 33.25 |
| ATOM | 11407 | CB | PHE | B | 695 | 78.192 | 61.644 | 49.575 | 1.00 | 34.62 |
| ATOM | 11408 | CG | PHE | B | 695 | 77.772 | 62.458 | 50.744 | 1.00 | 32.13 |
| ATOM | 11409 | CD1 | PHE | B | 695 | 77.832 | 63.813 | 50.707 | 1.00 | 31.70 |
| ATOM | 11410 | CD2 | PHE | B | 695 | 77.245 | 61.858 | 51.854 | 1.00 | 34.97 |
| ATOM | 11411 | CE1 | PHE | B | 695 | 77.404 | 64.565 | 51.762 | 1.00 | 32.49 |
| ATOM | 11412 | CE2 | PHE | B | 695 | 76.799 | 62.612 | 52.922 | 1.00 | 34.20 |
| ATOM | 11413 | CZ | PHE | B | 695 | 76.882 | 63.968 | 52.873 | 1.00 | 32.00 |
| ATOM | 11414 | N | LYS | B | 696 | 80.576 | 60.343 | 47.496 | 1.00 | 35.78 |
| ATOM | 11415 | CA | LYS | B | 696 | 80.986 | 59.173 | 46.713 | 1.00 | 36.63 |
| ATOM | 11416 | C | LYS | B | 696 | 82.089 | 58.418 | 47.417 | 1.00 | 36.37 |
| ATOM | 11417 | O | LYS | B | 696 | 82.193 | 57.220 | 47.250 | 1.00 | 36.72 |
| ATOM | 11418 | CB | LYS | B | 696 | 81.296 | 59.492 | 45.223 | 1.00 | 37.25 |
| ATOM | 11419 | CG | LYS | B | 696 | 82.562 | 60.245 | 44.839 | 1.00 | 40.92 |
| ATOM | 11420 | CD | LYS | B | 696 | 82.355 | 61.033 | 43.490 | 1.00 | 44.08 |
| ATOM | 11421 | CE | LYS | B | 696 | 83.601 | 61.872 | 43.107 | 1.00 | 46.36 |
| ATOM | 11422 | NZ | LYS | B | 696 | 83.396 | 62.925 | 42.052 | 1.00 | 45.25 |
| ATOM | 11423 | N | GLN | B | 697 | 82.839 | 59.094 | 48.290 | 1.00 | 36.14 |
| ATOM | 11424 | CA | GLN | B | 697 | 83.938 | 58.448 | 49.033 | 1.00 | 35.61 |
| ATOM | 11425 | C | GLN | B | 697 | 83.488 | 57.706 | 50.333 | 1.00 | 34.57 |
| ATOM | 11426 | O | GLN | B | 697 | 84.291 | 57.075 | 51.005 | 1.00 | 33.84 |
| ATOM | 11427 | CB | GLN | B | 697 | 84.984 | 59.505 | 49.417 | 1.00 | 35.75 |
| ATOM | 11428 | CG | GLN | B | 697 | 85.543 | 60.418 | 48.287 | 1.00 | 39.27 |
| ATOM | 11429 | CD | GLN | B | 697 | 86.447 | 61.543 | 48.835 | 1.00 | 44.90 |
| ATOM | 11430 | OE1 | GLN | B | 697 | 87.581 | 61.288 | 49.275 | 1.00 | 49.24 |
| ATOM | 11431 | NE2 | GLN | B | 697 | 85.929 | 62.783 | 48.843 | 1.00 | 47.40 |
| ATOM | 11432 | N | VAL | B | 698 | 82.210 | 57.755 | 50.681 | 1.00 | 33.48 |
| ATOM | 11433 | CA | VAL | B | 698 | 81.760 | 57.191 | 51.967 | 1.00 | 31.84 |
| ATOM | 11434 | C | VAL | B | 698 | 80.462 | 56.400 | 51.846 | 1.00 | 31.25 |
| ATOM | 11435 | O | VAL | B | 698 | 79.782 | 56.489 | 50.826 | 1.00 | 30.83 |
| ATOM | 11436 | CB | VAL | B | 698 | 81.539 | 58.324 | 52.958 | 1.00 | 31.73 |
| ATOM | 11437 | CG1 | VAL | B | 698 | 82.813 | 59.102 | 53.216 | 1.00 | 30.88 |
| ATOM | 11438 | CG2 | VAL | B | 698 | 80.491 | 59.258 | 52.452 | 1.00 | 32.77 |
| ATOM | 11439 | N | GLU | B | 699 | 80.145 | 55.598 | 52.869 | 1.00 | 30.04 |
| ATOM | 11440 | CA | GLU | B | 699 | 78.878 | 54.868 | 52.939 | 1.00 | 29.36 |
| ATOM | 11441 | C | GLU | B | 699 | 78.014 | 55.697 | 53.882 | 1.00 | 28.35 |
| ATOM | 11442 | O | GLU | B | 699 | 78.437 | 55.958 | 55.029 | 1.00 | 26.89 |
| ATOM | 11443 | CB | GLU | B | 699 | 79.026 | 53.449 | 53.492 | 1.00 | 29.28 |
| ATOM | 11444 | CG | GLU | B | 699 | 79.896 | 52.499 | 52.658 | 1.00 | 34.79 |
| ATOM | 11445 | CD | GLU | B | 699 | 80.472 | 51.307 | 53.466 | 1.00 | 41.12 |
| ATOM | 11446 | OE1 | GLU | B | 699 | 79.746 | 50.719 | 54.317 | 1.00 | 44.68 |
| ATOM | 11447 | OE2 | GLU | B | 699 | 81.662 | 50.940 | 53.250 | 1.00 | 45.23 |
| ATOM | 11448 | N | TYR | B | 700 | 76.839 | 56.121 | 53.394 | 1.00 | 26.10 |
| ATOM | 11449 | CA | TYR | B | 700 | 75.930 | 56.964 | 54.143 | 1.00 | 25.87 |
| ATOM | 11450 | C | TYR | B | 700 | 74.560 | 56.293 | 54.351 | 1.00 | 25.50 |
| ATOM | 11451 | O | TYR | B | 700 | 73.997 | 55.657 | 53.418 | 1.00 | 24.19 |
| ATOM | 11452 | CB | TYR | B | 700 | 75.764 | 58.238 | 53.352 | 1.00 | 26.07 |
| ATOM | 11453 | CG | TYR | B | 700 | 74.997 | 59.418 | 53.933 | 1.00 | 24.42 |
| ATOM | 11454 | CD1 | TYR | B | 700 | 75.240 | 59.934 | 55.215 | 1.00 | 23.88 |
| ATOM | 11455 | CD2 | TYR | B | 700 | 74.114 | 60.085 | 53.138 | 1.00 | 20.95 |
| ATOM | 11456 | CE1 | TYR | B | 700 | 74.579 | 61.067 | 55.649 | 1.00 | 21.97 |
| ATOM | 11457 | CE2 | TYR | B | 700 | 73.453 | 61.175 | 53.560 | 1.00 | 22.57 |
| ATOM | 11458 | CZ | TYR | B | 700 | 73.685 | 61.683 | 54.807 | 1.00 | 24.01 |
| ATOM | 11459 | OH | TYR | B | 700 | 72.979 | 62.799 | 55.149 | 1.00 | 24.24 |
| ATOM | 11460 | N | LEU | B | 701 | 74.078 | 56.390 | 55.594 | 1.00 | 23.78 |
| ATOM | 11461 | CA | LEU | B | 701 | 72.740 | 56.002 | 55.958 | 1.00 | 23.60 |
| ATOM | 11462 | C | LEU | B | 701 | 72.050 | 57.268 | 56.454 | 1.00 | 23.98 |
| ATOM | 11463 | O | LEU | B | 701 | 72.565 | 57.992 | 57.353 | 1.00 | 23.64 |
| ATOM | 11464 | CB | LEU | B | 701 | 72.732 | 54.920 | 57.007 | 1.00 | 23.26 |
| ATOM | 11465 | CG | LEU | B | 701 | 71.381 | 54.467 | 57.569 | 1.00 | 24.75 |
| ATOM | 11466 | CD1 | LEU | B | 701 | 70.344 | 54.071 | 56.500 | 1.00 | 26.22 |
| ATOM | 11467 | CD2 | LEU | B | 701 | 71.595 | 53.318 | 58.502 | 1.00 | 22.99 |
| ATOM | 11468 | N | LEU | B | 702 | 70.912 | 57.536 | 55.820 | 1.00 | 23.77 |
| ATOM | 11469 | CA | LEU | B | 702 | 70.125 | 58.717 | 56.064 | 1.00 | 23.66 |
| ATOM | 11470 | C | LEU | B | 702 | 68.759 | 58.265 | 56.525 | 1.00 | 22.87 |
| ATOM | 11471 | O | LEU | B | 702 | 68.089 | 57.440 | 55.863 | 1.00 | 22.89 |
| ATOM | 11472 | CB | LEU | B | 702 | 70.068 | 59.512 | 54.769 | 1.00 | 23.91 |
| ATOM | 11473 | CG | LEU | B | 702 | 69.135 | 60.686 | 54.696 | 1.00 | 23.94 |
| ATOM | 11474 | CD1 | LEU | B | 702 | 69.731 | 61.768 | 55.572 | 1.00 | 26.82 |
| ATOM | 11475 | CD2 | LEU | B | 702 | 69.079 | 61.125 | 53.298 | 1.00 | 25.37 |
| ATOM | 11476 | N | ILE | B | 703 | 68.356 | 58.761 | 57.680 | 1.00 | 22.92 |
| ATOM | 11477 | CA | ILE | B | 703 | 67.108 | 58.346 | 58.299 | 1.00 | 23.25 |
| ATOM | 11478 | C | ILE | B | 703 | 66.308 | 59.532 | 58.745 | 1.00 | 23.23 |
| ATOM | 11479 | O | ILE | B | 703 | 66.868 | 60.463 | 59.318 | 1.00 | 23.16 |
| ATOM | 11480 | CB | ILE | B | 703 | 67.375 | 57.497 | 59.513 | 1.00 | 24.17 |
| ATOM | 11481 | CG1 | ILE | B | 703 | 68.285 | 56.354 | 59.156 | 1.00 | 25.71 |
| ATOM | 11482 | CG2 | ILE | B | 703 | 66.057 | 56.981 | 60.096 | 1.00 | 23.96 |
| ATOM | 11483 | CD1 | ILE | B | 703 | 68.895 | 55.693 | 60.368 | 1.00 | 27.51 |
| ATOM | 11484 | N | HIS | B | 704 | 64.989 | 59.498 | 58.510 | 1.00 | 22.56 |
| ATOM | 11485 | CA | HIS | B | 704 | 64.120 | 60.615 | 58.931 | 1.00 | 21.90 |
| ATOM | 11486 | C | HIS | B | 704 | 62.684 | 60.148 | 59.135 | 1.00 | 21.79 |
| ATOM | 11487 | O | HIS | B | 704 | 62.214 | 59.265 | 58.411 | 1.00 | 23.20 |
| ATOM | 11488 | CB | HIS | B | 704 | 64.173 | 61.714 | 57.891 | 1.00 | 20.48 |
| ATOM | 11489 | CG | HIS | B | 704 | 64.148 | 63.084 | 58.455 | 1.00 | 20.84 |
| ATOM | 11490 | ND1 | HIS | B | 704 | 65.091 | 64.034 | 58.125 | 1.00 | 22.71 |
| ATOM | 11491 | CD2 | HIS | B | 704 | 63.285 | 63.687 | 59.309 | 1.00 | 19.35 |
| ATOM | 11492 | CE1 | HIS | B | 704 | 64.812 | 65.163 | 58.766 | 1.00 | 22.05 |
| ATOM | 11493 | NE2 | HIS | B | 704 | 63.738 | 64.969 | 59.513 | 1.00 | 17.00 |
| ATOM | 11494 | N | GLY | B | 705 | 61.992 | 60.719 | 60.115 | 1.00 | 21.89 |
| ATOM | 11495 | CA | GLY | B | 705 | 60.612 | 60.378 | 60.420 | 1.00 | 21.24 |
| ATOM | 11496 | C | GLY | B | 705 | 59.716 | 61.273 | 59.587 | 1.00 | 22.48 |
| ATOM | 11497 | O | GLY | B | 705 | 60.002 | 62.462 | 59.447 | 1.00 | 22.15 |
| ATOM | 11498 | N | THR | B | 706 | 58.628 | 60.740 | 59.020 | 1.00 | 22.26 |
| ATOM | 11499 | CA | THR | B | 706 | 57.878 | 61.557 | 58.086 | 1.00 | 22.34 |
| ATOM | 11500 | C | THR | B | 706 | 56.952 | 62.542 | 58.755 | 1.00 | 21.18 |
| ATOM | 11501 | O | THR | B | 706 | 56.578 | 63.486 | 58.121 | 1.00 | 20.70 |
| ATOM | 11502 | CB | THR | B | 706 | 57.077 | 60.724 | 57.052 | 1.00 | 22.35 |
| ATOM | 11503 | OG1 | THR | B | 706 | 56.131 | 59.951 | 57.740 | 1.00 | 20.72 |
| ATOM | 11504 | CG2 | THR | B | 706 | 57.913 | 59.659 | 56.425 | 1.00 | 24.38 |
| ATOM | 11505 | N | ALA | B | 707 | 56.622 | 62.369 | 60.022 | 1.00 | 21.54 |
| ATOM | 11506 | CA | ALA | B | 707 | 55.778 | 63.352 | 60.713 | 1.00 | 22.57 |
| ATOM | 11507 | C | ALA | B | 707 | 56.614 | 64.271 | 61.623 | 1.00 | 23.06 |
| ATOM | 11508 | O | ALA | B | 707 | 56.133 | 64.673 | 62.665 | 1.00 | 23.66 |
| ATOM | 11509 | CB | ALA | B | 707 | 54.675 | 62.642 | 61.583 | 1.00 | 22.37 |
| ATOM | 11510 | N | ASP | B | 708 | 57.850 | 64.573 | 61.232 | 1.00 | 23.56 |
| ATOM | 11511 | CA | ASP | B | 708 | 58.731 | 65.467 | 61.996 | 1.00 | 23.27 |
| ATOM | 11512 | C | ASP | B | 708 | 58.293 | 66.920 | 61.844 | 1.00 | 22.78 |
| ATOM | 11513 | O | ASP | B | 708 | 58.520 | 67.556 | 60.809 | 1.00 | 22.16 |
| ATOM | 11514 | CB | ASP | B | 708 | 60.165 | 65.284 | 61.523 | 1.00 | 23.23 |
| ATOM | 11515 | CG | ASP | B | 708 | 61.218 | 65.711 | 62.566 | 1.00 | 23.33 |
| ATOM | 11516 | OD1 | ASP | B | 708 | 60.975 | 66.664 | 63.341 | 1.00 | 18.66 |
| ATOM | 11517 | OD2 | ASP | B | 708 | 62.319 | 65.109 | 62.642 | 1.00 | 17.00 |
| ATOM | 11518 | N | ASP | B | 709 | 57.646 | 67.420 | 62.881 | 1.00 | 22.06 |
| ATOM | 11519 | CA | ASP | B | 709 | 57.170 | 68.793 | 62.954 | 1.00 | 22.26 |
| ATOM | 11520 | C | ASP | B | 709 | 58.278 | 69.756 | 63.284 | 1.00 | 21.89 |
| ATOM | 11521 | O | ASP | B | 709 | 58.053 | 70.941 | 63.221 | 1.00 | 21.79 |
| ATOM | 11522 | CB | ASP | B | 709 | 56.140 | 68.959 | 64.097 | 1.00 | 21.75 |
| ATOM | 11523 | CG | ASP | B | 709 | 56.670 | 68.443 | 65.449 | 1.00 | 23.45 |
| ATOM | 11524 | OD1 | ASP | B | 709 | 56.818 | 67.219 | 65.622 | 1.00 | 21.03 |
| ATOM | 11525 | OD2 | ASP | B | 709 | 56.958 | 69.184 | 66.403 | 1.00 | 24.68 |
| ATOM | 11526 | N | ASN | B | 710 | 59.432 | 69.235 | 63.702 | 1.00 | 22.00 |
| ATOM | 11527 | CA | ASN | B | 710 | 60.568 | 70.042 | 64.178 | 1.00 | 21.88 |
| ATOM | 11528 | C | ASN | B | 710 | 61.607 | 70.273 | 63.084 | 1.00 | 20.54 |
| ATOM | 11529 | O | ASN | B | 710 | 61.738 | 71.383 | 62.555 | 1.00 | 20.05 |
| ATOM | 11530 | CB | ASN | B | 710 | 61.128 | 69.354 | 65.446 | 1.00 | 21.90 |
| ATOM | 11531 | CG | ASN | B | 710 | 62.126 | 70.223 | 66.259 | 1.00 | 24.00 |
| ATOM | 11532 | OD1 | ASN | B | 710 | 62.401 | 69.902 | 67.439 | 1.00 | 30.50 |
| ATOM | 11533 | ND2 | ASN | B | 710 | 62.686 | 71.259 | 65.656 | 1.00 | 20.43 |
| ATOM | 11534 | N | VAL | B | 711 | 62.377 | 69.244 | 62.775 | 1.00 | 20.79 |
| ATOM | 11535 | CA | VAL | B | 711 | 63.293 | 69.282 | 61.648 | 1.00 | 21.00 |
| ATOM | 11536 | C | VAL | B | 711 | 62.507 | 68.571 | 60.563 | 1.00 | 21.12 |
| ATOM | 11537 | O | VAL | B | 711 | 62.433 | 67.346 | 60.537 | 1.00 | 21.84 |
| ATOM | 11538 | CB | VAL | B | 711 | 64.593 | 68.498 | 61.927 | 1.00 | 21.08 |
| ATOM | 11539 | CG1 | VAL | B | 711 | 65.469 | 68.390 | 60.671 | 1.00 | 22.87 |
| ATOM | 11540 | CG2 | VAL | B | 711 | 65.347 | 69.124 | 63.093 | 1.00 | 24.03 |
| ATOM | 11541 | N | HIS | B | 712 | 61.924 | 69.335 | 59.655 | 1.00 | 21.51 |
| ATOM | 11542 | CA | HIS | B | 712 | 60.994 | 68.751 | 58.695 | 1.00 | 22.37 |
| ATOM | 11543 | C | HIS | B | 712 | 61.604 | 67.679 | 57.802 | 1.00 | 22.30 |
| ATOM | 11544 | O | HIS | B | 712 | 62.793 | 67.747 | 57.456 | 1.00 | 23.20 |
| ATOM | 11545 | CB | HIS | B | 712 | 60.320 | 69.899 | 57.919 | 1.00 | 22.00 |
| ATOM | 11546 | CG | HIS | B | 712 | 59.629 | 70.855 | 58.827 | 1.00 | 22.77 |
| ATOM | 11547 | ND1 | HIS | B | 712 | 59.533 | 72.206 | 58.579 | 1.00 | 24.42 |
| ATOM | 11548 | CD2 | HIS | B | 712 | 59.053 | 70.651 | 60.036 | 1.00 | 22.52 |
| ATOM | 11549 | CE1 | HIS | B | 712 | 58.909 | 72.791 | 59.584 | 1.00 | 25.18 |
| ATOM | 11550 | NE2 | HIS | B | 712 | 58.583 | 71.865 | 60.472 | 1.00 | 24.45 |
| ATOM | 11551 | N | PHE | B | 713 | 60.802 | 66.690 | 57.421 | 1.00 | 21.85 |
| ATOM | 11552 | CA | PHE | B | 713 | 61.291 | 65.610 | 56.547 | 1.00 | 21.49 |
| ATOM | 11553 | C | PHE | B | 713 | 61.848 | 66.203 | 55.254 | 1.00 | 22.74 |
| ATOM | 11554 | O | PHE | B | 713 | 62.771 | 65.650 | 54.660 | 1.00 | 22.10 |
| ATOM | 11555 | CB | PHE | B | 713 | 60.148 | 64.644 | 56.225 | 1.00 | 22.62 |
| ATOM | 11556 | CG | PHE | B | 713 | 60.535 | 63.528 | 55.349 | 1.00 | 20.27 |
| ATOM | 11557 | CD1 | PHE | B | 713 | 61.023 | 62.357 | 55.888 | 1.00 | 20.13 |
| ATOM | 11558 | CD2 | PHE | B | 713 | 60.449 | 63.658 | 53.967 | 1.00 | 25.42 |
| ATOM | 11559 | CE1 | PHE | B | 713 | 61.407 | 61.317 | 55.077 | 1.00 | 23.04 |
| ATOM | 11560 | CE2 | PHE | B | 713 | 60.838 | 62.602 | 53.132 | 1.00 | 25.22 |
| ATOM | 11561 | CZ | PHE | B | 713 | 61.323 | 61.431 | 53.704 | 1.00 | 24.08 |
| ATOM | 11562 | N | GLN | B | 714 | 61.259 | 67.322 | 54.817 | 1.00 | 22.17 |
| ATOM | 11563 | CA | GLN | B | 714 | 61.814 | 68.102 | 53.726 | 1.00 | 22.19 |
| ATOM | 11564 | C | GLN | B | 714 | 63.327 | 68.117 | 53.726 | 1.00 | 22.48 |
| ATOM | 11565 | O | GLN | B | 714 | 63.965 | 67.952 | 52.677 | 1.00 | 21.65 |
| ATOM | 11566 | CB | GLN | B | 714 | 61.342 | 69.560 | 53.875 | 1.00 | 22.19 |
| ATOM | 11567 | CG | GLN | B | 714 | 62.190 | 70.636 | 53.144 | 1.00 | 23.25 |
| ATOM | 11568 | CD | GLN | B | 714 | 61.742 | 72.058 | 53.493 | 1.00 | 23.78 |
| ATOM | 11569 | OE1 | GLN | B | 714 | 61.589 | 72.367 | 54.663 | 1.00 | 24.99 |
| ATOM | 11570 | NE2 | GLN | B | 714 | 61.585 | 72.926 | 52.491 | 1.00 | 24.29 |
| ATOM | 11571 | N | GLN | B | 715 | 63.899 | 68.307 | 54.919 | 1.00 | 21.92 |
| ATOM | 11572 | CA | GLN | B | 715 | 65.307 | 68.551 | 55.037 | 1.00 | 21.53 |
| ATOM | 11573 | C | GLN | B | 715 | 66.092 | 67.407 | 54.451 | 1.00 | 21.26 |
| ATOM | 11574 | O | GLN | B | 715 | 67.033 | 67.631 | 53.710 | 1.00 | 21.16 |
| ATOM | 11575 | CB | GLN | B | 715 | 65.715 | 68.855 | 56.500 | 1.00 | 21.69 |
| ATOM | 11576 | CG | GLN | B | 715 | 65.009 | 70.103 | 57.110 | 1.00 | 22.09 |
| ATOM | 11577 | CD | GLN | B | 715 | 65.926 | 71.119 | 57.862 | 1.00 | 19.03 |
| ATOM | 11578 | OE1 | GLN | B | 715 | 65.515 | 71.748 | 58.868 | 1.00 | 23.44 |
| ATOM | 11579 | NE2 | GLN | B | 715 | 67.094 | 71.293 | 57.379 | 1.00 | 16.51 |
| ATOM | 11580 | N | SER | B | 716 | 65.733 | 66.182 | 54.821 | 1.00 | 22.18 |
| ATOM | 11581 | CA | SER | B | 716 | 66.418 | 65.010 | 54.317 | 1.00 | 22.37 |
| ATOM | 11582 | C | SER | B | 716 | 65.971 | 64.742 | 52.868 | 1.00 | 23.64 |
| ATOM | 11583 | O | SER | B | 716 | 66.707 | 64.131 | 52.080 | 1.00 | 24.04 |
| ATOM | 11584 | CB | SER | B | 716 | 66.092 | 63.783 | 55.147 | 1.00 | 22.45 |
| ATOM | 11585 | OG | SER | B | 716 | 66.807 | 63.738 | 56.376 | 1.00 | 20.69 |
| ATOM | 11586 | N | ALA | B | 717 | 64.771 | 65.188 | 52.523 | 1.00 | 22.98 |
| ATOM | 11587 | CA | ALA | B | 717 | 64.281 | 64.913 | 51.188 | 1.00 | 24.31 |
| ATOM | 11588 | C | ALA | B | 717 | 65.196 | 65.644 | 50.212 | 1.00 | 23.91 |
| ATOM | 11589 | O | ALA | B | 717 | 65.492 | 65.147 | 49.172 | 1.00 | 24.21 |
| ATOM | 11590 | CB | ALA | B | 717 | 62.834 | 65.365 | 51.004 | 1.00 | 23.00 |
| ATOM | 11591 | N | GLN | B | 718 | 65.609 | 66.843 | 50.576 | 1.00 | 24.92 |
| ATOM | 11592 | CA | GLN | B | 718 | 66.521 | 67.596 | 49.741 | 1.00 | 25.91 |
| ATOM | 11593 | C | GLN | B | 718 | 67.924 | 66.982 | 49.769 | 1.00 | 25.69 |
| ATOM | 11594 | O | GLN | B | 718 | 68.618 | 67.111 | 48.814 | 1.00 | 26.05 |
| ATOM | 11595 | CB | GLN | B | 718 | 66.516 | 69.059 | 50.134 | 1.00 | 25.77 |
| ATOM | 11596 | CG | GLN | B | 718 | 65.141 | 69.747 | 49.884 | 1.00 | 28.26 |
| ATOM | 11597 | CD | GLN | B | 718 | 64.906 | 70.071 | 48.413 | 1.00 | 28.48 |
| ATOM | 11598 | OE1 | GLN | B | 718 | 65.645 | 69.614 | 47.565 | 1.00 | 30.00 |
| ATOM | 11599 | NE2 | GLN | B | 718 | 63.916 | 70.886 | 48.127 | 1.00 | 29.10 |
| ATOM | 11600 | N | ILE | B | 719 | 68.343 | 66.320 | 50.844 | 1.00 | 25.52 |
| ATOM | 11601 | CA | ILE | B | 719 | 69.634 | 65.647 | 50.809 | 1.00 | 25.65 |
| ATOM | 11602 | C | ILE | B | 719 | 69.630 | 64.487 | 49.777 | 1.00 | 25.82 |
| ATOM | 11603 | O | ILE | B | 719 | 70.555 | 64.345 | 48.957 | 1.00 | 25.97 |
| ATOM | 11604 | CB | ILE | B | 719 | 70.022 | 65.082 | 52.172 | 1.00 | 25.32 |
| ATOM | 11605 | CG1 | ILE | B | 719 | 70.333 | 66.183 | 53.160 | 1.00 | 25.43 |
| ATOM | 11606 | CG2 | ILE | B | 719 | 71.289 | 64.246 | 52.025 | 1.00 | 25.41 |
| ATOM | 11607 | CD1 | ILE | B | 719 | 70.485 | 65.688 | 54.569 | 1.00 | 22.72 |
| ATOM | 11608 | N | SER | B | 720 | 68.615 | 63.632 | 49.831 | 1.00 | 25.27 |
| ATOM | 11609 | CA | SER | B | 720 | 68.550 | 62.505 | 48.886 | 1.00 | 24.93 |
| ATOM | 11610 | C | SER | B | 720 | 68.476 | 62.969 | 47.430 | 1.00 | 25.54 |
| ATOM | 11611 | O | SER | B | 720 | 69.115 | 62.383 | 46.568 | 1.00 | 24.83 |
| ATOM | 11612 | CB | SER | B | 720 | 67.363 | 61.583 | 49.220 | 1.00 | 25.30 |
| ATOM | 11613 | OG | SER | B | 720 | 66.126 | 62.268 | 49.085 | 1.00 | 22.78 |
| ATOM | 11614 | N | LYS | B | 721 | 67.722 | 64.042 | 47.177 | 1.00 | 26.85 |
| ATOM | 11615 | CA | LYS | B | 721 | 67.559 | 64.596 | 45.848 | 1.00 | 27.84 |
| ATOM | 11616 | C | LYS | B | 721 | 68.939 | 65.015 | 45.340 | 1.00 | 28.04 |
| ATOM | 11617 | O | LYS | B | 721 | 69.278 | 64.750 | 44.207 | 1.00 | 26.90 |
| ATOM | 11618 | CB | LYS | B | 721 | 66.580 | 65.809 | 45.846 | 1.00 | 29.05 |
| ATOM | 11619 | CG | LYS | B | 721 | 66.109 | 66.372 | 44.416 | 1.00 | 31.60 |
| ATOM | 11620 | CD | LYS | B | 721 | 65.107 | 67.681 | 44.415 | 1.00 | 35.20 |
| ATOM | 11621 | CE | LYS | B | 721 | 64.834 | 68.185 | 42.881 | 1.00 | 39.27 |
| ATOM | 11622 | NZ | LYS | B | 721 | 64.418 | 69.677 | 42.463 | 1.00 | 33.78 |
| ATOM | 11623 | N | ALA | B | 722 | 69.757 | 65.632 | 46.191 | 1.00 | 28.55 |
| ATOM | 11624 | CA | ALA | B | 722 | 71.034 | 66.136 | 45.715 | 1.00 | 27.85 |
| ATOM | 11625 | C | ALA | B | 722 | 71.948 | 64.993 | 45.455 | 1.00 | 27.50 |
| ATOM | 11626 | O | ALA | B | 722 | 72.633 | 64.981 | 44.439 | 1.00 | 29.51 |
| ATOM | 11627 | CB | ALA | B | 722 | 71.642 | 67.131 | 46.682 | 1.00 | 27.80 |
| ATOM | 11628 | N | LEU | B | 723 | 71.953 | 64.008 | 46.334 | 1.00 | 26.95 |
| ATOM | 11629 | CA | LEU | B | 723 | 72.780 | 62.851 | 46.130 | 1.00 | 26.84 |
| ATOM | 11630 | C | LEU | B | 723 | 72.343 | 62.168 | 44.830 | 1.00 | 28.27 |
| ATOM | 11631 | O | LEU | B | 723 | 73.156 | 61.687 | 44.071 | 1.00 | 28.67 |
| ATOM | 11632 | CB | LEU | B | 723 | 72.695 | 61.897 | 47.327 | 1.00 | 27.02 |
| ATOM | 11633 | CG | LEU | B | 723 | 73.238 | 62.436 | 48.685 | 1.00 | 28.19 |
| ATOM | 11634 | CD1 | LEU | B | 723 | 72.926 | 61.499 | 49.870 | 1.00 | 27.76 |
| ATOM | 11635 | CD2 | LEU | B | 723 | 74.722 | 62.713 | 48.607 | 1.00 | 26.33 |
| ATOM | 11636 | N | VAL | B | 724 | 71.049 | 62.114 | 44.569 | 1.00 | 29.67 |
| ATOM | 11637 | CA | VAL | B | 724 | 70.570 | 61.507 | 43.334 | 1.00 | 30.31 |
| ATOM | 11638 | C | VAL | B | 724 | 71.007 | 62.366 | 42.129 | 1.00 | 30.78 |
| ATOM | 11639 | O | VAL | B | 724 | 71.481 | 61.826 | 41.151 | 1.00 | 29.49 |
| ATOM | 11640 | CB | VAL | B | 724 | 69.028 | 61.285 | 43.353 | 1.00 | 29.84 |
| ATOM | 11641 | CG1 | VAL | B | 724 | 68.515 | 60.949 | 41.936 | 1.00 | 28.94 |
| ATOM | 11642 | CG2 | VAL | B | 724 | 68.653 | 60.141 | 44.369 | 1.00 | 27.98 |
| ATOM | 11643 | N | ASP | B | 725 | 70.861 | 63.692 | 42.196 | 1.00 | 31.74 |
| ATOM | 11644 | CA | ASP | B | 725 | 71.261 | 64.520 | 41.053 | 1.00 | 33.00 |
| ATOM | 11645 | C | ASP | B | 725 | 72.725 | 64.245 | 40.652 | 1.00 | 33.14 |
| ATOM | 11646 | O | ASP | B | 725 | 73.100 | 64.358 | 39.488 | 1.00 | 33.52 |
| ATOM | 11647 | CB | ASP | B | 725 | 71.107 | 66.035 | 41.327 | 1.00 | 33.88 |
| ATOM | 11648 | CG | ASP | B | 725 | 69.630 | 66.495 | 41.493 | 1.00 | 36.92 |
| ATOM | 11649 | OD1 | ASP | B | 725 | 68.679 | 65.847 | 40.967 | 1.00 | 36.79 |
| ATOM | 11650 | OD2 | ASP | B | 725 | 69.338 | 67.524 | 42.156 | 1.00 | 41.86 |
| ATOM | 11651 | N | VAL | B | 726 | 73.543 | 63.832 | 41.610 | 1.00 | 32.56 |
| ATOM | 11652 | CA | VAL | B | 726 | 74.948 | 63.621 | 41.354 | 1.00 | 32.51 |
| ATOM | 11653 | C | VAL | B | 726 | 75.397 | 62.154 | 41.186 | 1.00 | 31.29 |
| ATOM | 11654 | O | VAL | B | 726 | 76.592 | 61.875 | 41.065 | 1.00 | 29.89 |
| ATOM | 11655 | CB | VAL | B | 726 | 75.709 | 64.370 | 42.493 | 1.00 | 33.37 |
| ATOM | 11656 | CG1 | VAL | B | 726 | 76.678 | 63.529 | 43.189 | 1.00 | 34.52 |
| ATOM | 11657 | CG2 | VAL | B | 726 | 76.328 | 65.596 | 41.953 | 1.00 | 35.51 |
| ATOM | 11658 | N | GLY | B | 727 | 74.465 | 61.208 | 41.153 | 1.00 | 29.96 |
| ATOM | 11659 | CA | GLY | B | 727 | 74.844 | 59.805 | 40.982 | 1.00 | 29.57 |
| ATOM | 11660 | C | GLY | B | 727 | 75.622 | 59.125 | 42.115 | 1.00 | 29.81 |
| ATOM | 11661 | O | GLY | B | 727 | 76.456 | 58.239 | 41.881 | 1.00 | 29.75 |
| ATOM | 11662 | N | VAL | B | 728 | 75.343 | 59.504 | 43.358 | 1.00 | 29.64 |
| ATOM | 11663 | CA | VAL | B | 728 | 75.964 | 58.889 | 44.526 | 1.00 | 29.43 |
| ATOM | 11664 | C | VAL | B | 728 | 75.019 | 57.918 | 45.186 | 1.00 | 29.95 |
| ATOM | 11665 | O | VAL | B | 728 | 73.850 | 58.238 | 45.466 | 1.00 | 29.36 |
| ATOM | 11666 | CB | VAL | B | 728 | 76.286 | 59.943 | 45.591 | 1.00 | 29.54 |
| ATOM | 11667 | CG1 | VAL | B | 728 | 76.716 | 59.313 | 46.853 | 1.00 | 29.43 |
| ATOM | 11668 | CG2 | VAL | B | 728 | 77.369 | 60.889 | 45.092 | 1.00 | 33.38 |
| ATOM | 11669 | N | ASP | B | 729 | 75.525 | 56.740 | 45.464 | 1.00 | 29.47 |
| ATOM | 11670 | CA | ASP | B | 729 | 74.742 | 55.769 | 46.124 | 1.00 | 30.28 |
| ATOM | 11671 | C | ASP | B | 729 | 74.880 | 55.871 | 47.661 | 1.00 | 29.90 |
| ATOM | 11672 | O | ASP | B | 729 | 75.933 | 56.207 | 48.225 | 1.00 | 29.99 |
| ATOM | 11673 | CB | ASP | B | 729 | 75.077 | 54.373 | 45.594 | 1.00 | 30.38 |
| ATOM | 11674 | CG | ASP | B | 729 | 74.072 | 53.317 | 46.058 | 1.00 | 30.43 |
| ATOM | 11675 | OD1 | ASP | B | 729 | 72.852 | 53.554 | 45.946 | 1.00 | 30.84 |
| ATOM | 11676 | OD2 | ASP | B | 729 | 74.410 | 52.231 | 46.552 | 1.00 | 32.04 |
| ATOM | 11677 | N | PHE | B | 730 | 73.770 | 55.603 | 48.328 | 1.00 | 29.05 |
| ATOM | 11678 | CA | PHE | B | 730 | 73.667 | 55.756 | 49.761 | 1.00 | 28.20 |
| ATOM | 11679 | C | PHE | B | 730 | 72.502 | 54.900 | 50.194 | 1.00 | 28.15 |
| ATOM | 11680 | O | PHE | B | 730 | 71.750 | 54.418 | 49.346 | 1.00 | 27.29 |
| ATOM | 11681 | CB | PHE | B | 730 | 73.443 | 57.234 | 50.115 | 1.00 | 27.96 |
| ATOM | 11682 | CG | PHE | B | 730 | 72.176 | 57.823 | 49.561 | 1.00 | 29.42 |
| ATOM | 11683 | CD1 | PHE | B | 730 | 72.127 | 58.314 | 48.280 | 1.00 | 30.54 |
| ATOM | 11684 | CD2 | PHE | B | 730 | 71.033 | 57.899 | 50.342 | 1.00 | 33.30 |
| ATOM | 11685 | CE1 | PHE | B | 730 | 70.953 | 58.860 | 47.749 | 1.00 | 32.34 |
| ATOM | 11686 | CE2 | PHE | B | 730 | 69.851 | 58.448 | 49.834 | 1.00 | 33.34 |
| ATOM | 11687 | CZ | PHE | B | 730 | 69.818 | 58.931 | 48.532 | 1.00 | 33.29 |
| ATOM | 11688 | N | GLN | B | 731 | 72.352 | 54.706 | 51.498 | 1.00 | 28.88 |
| ATOM | 11689 | CA | GLN | B | 731 | 71.275 | 53.904 | 52.066 | 1.00 | 29.87 |
| ATOM | 11690 | C | GLN | B | 731 | 70.330 | 54.840 | 52.761 | 1.00 | 29.70 |
| ATOM | 11691 | O | GLN | B | 731 | 70.748 | 55.886 | 53.318 | 1.00 | 29.43 |
| ATOM | 11692 | CB | GLN | B | 731 | 71.785 | 52.923 | 53.115 | 1.00 | 31.26 |
| ATOM | 11693 | CG | GLN | B | 731 | 73.154 | 52.361 | 52.838 | 1.00 | 35.94 |
| ATOM | 11694 | CD | GLN | B | 731 | 73.103 | 50.988 | 52.257 | 1.00 | 42.08 |
| ATOM | 11695 | OE1 | GLN | B | 731 | 72.304 | 50.713 | 51.336 | 1.00 | 42.62 |
| ATOM | 11696 | NE2 | GLN | B | 731 | 73.959 | 50.091 | 52.793 | 1.00 | 44.08 |
| ATOM | 11697 | N | ALA | B | 732 | 69.067 | 54.439 | 52.801 | 1.00 | 29.28 |
| ATOM | 11698 | CA | ALA | B | 732 | 68.023 | 55.293 | 53.361 | 1.00 | 29.21 |
| ATOM | 11699 | C | ALA | B | 732 | 66.951 | 54.520 | 54.129 | 1.00 | 29.27 |
| ATOM | 11700 | O | ALA | B | 732 | 66.761 | 53.313 | 53.925 | 1.00 | 29.99 |
| ATOM | 11701 | CB | ALA | B | 732 | 67.375 | 56.145 | 52.274 | 1.00 | 28.18 |
| ATOM | 11702 | N | MET | B | 733 | 66.303 | 55.245 | 55.035 | 1.00 | 28.87 |
| ATOM | 11703 | CA | MET | B | 733 | 65.145 | 54.760 | 55.769 | 1.00 | 28.98 |
| ATOM | 11704 | C | MET | B | 733 | 64.260 | 55.891 | 56.248 | 1.00 | 27.35 |
| ATOM | 11705 | O | MET | B | 733 | 64.706 | 56.747 | 57.047 | 1.00 | 24.40 |
| ATOM | 11706 | CB | MET | B | 733 | 65.570 | 54.042 | 57.018 | 1.00 | 29.60 |
| ATOM | 11707 | CG | MET | B | 733 | 65.317 | 52.628 | 56.979 | 1.00 | 35.13 |
| ATOM | 11708 | SD | MET | B | 733 | 63.595 | 52.220 | 56.963 | 1.00 | 36.43 |
| ATOM | 11709 | CE | MET | B | 733 | 63.819 | 50.655 | 56.702 | 1.00 | 38.49 |
| ATOM | 11710 | N | TRP | B | 734 | 63.006 | 55.881 | 55.802 | 1.00 | 25.36 |
| ATOM | 11711 | CA | TRP | B | 734 | 62.025 | 56.798 | 56.369 | 1.00 | 24.63 |
| ATOM | 11712 | C | TRP | B | 734 | 61.279 | 56.066 | 57.480 | 1.00 | 23.31 |
| ATOM | 11713 | O | TRP | B | 734 | 61.258 | 54.858 | 57.475 | 1.00 | 21.83 |
| ATOM | 11714 | CB | TRP | B | 734 | 61.059 | 57.308 | 55.302 | 1.00 | 24.60 |
| ATOM | 11715 | CG | TRP | B | 734 | 60.014 | 56.283 | 54.885 | 1.00 | 25.26 |
| ATOM | 11716 | CD1 | TRP | B | 734 | 58.862 | 55.980 | 55.548 | 1.00 | 25.43 |
| ATOM | 11717 | CD2 | TRP | B | 734 | 60.044 | 55.451 | 53.732 | 1.00 | 24.00 |
| ATOM | 11718 | NE1 | TRP | B | 734 | 58.155 | 55.014 | 54.867 | 1.00 | 26.11 |
| ATOM | 11719 | CE2 | TRP | B | 734 | 58.861 | 54.666 | 53.745 | 1.00 | 25.69 |
| ATOM | 11720 | CE3 | TRP | B | 734 | 60.937 | 55.298 | 52.671 | 1.00 | 26.17 |
| ATOM | 11721 | CZ2 | TRP | B | 734 | 58.567 | 53.718 | 52.762 | 1.00 | 22.58 |
| ATOM | 11722 | CZ3 | TRP | B | 734 | 60.641 | 54.373 | 51.670 | 1.00 | 26.40 |
| ATOM | 11723 | CH2 | TRP | B | 734 | 59.459 | 53.597 | 51.725 | 1.00 | 27.21 |
| ATOM | 11724 | N | TYR | B | 735 | 60.727 | 56.795 | 58.464 | 1.00 | 23.19 |
| ATOM | 11725 | CA | TYR | B | 735 | 59.876 | 56.163 | 59.519 | 1.00 | 22.65 |
| ATOM | 11726 | C | TYR | B | 735 | 58.507 | 56.813 | 59.510 | 1.00 | 21.88 |
| ATOM | 11727 | O | TYR | B | 735 | 58.306 | 57.948 | 59.953 | 1.00 | 19.78 |
| ATOM | 11728 | CB | TYR | B | 735 | 60.472 | 56.186 | 60.909 | 1.00 | 22.20 |
| ATOM | 11729 | CG | TYR | B | 735 | 61.503 | 55.148 | 61.079 | 1.00 | 21.04 |
| ATOM | 11730 | CD1 | TYR | B | 735 | 61.145 | 53.851 | 61.385 | 1.00 | 22.90 |
| ATOM | 11731 | CD2 | TYR | B | 735 | 62.821 | 55.419 | 60.816 | 1.00 | 22.29 |
| ATOM | 11732 | CE1 | TYR | B | 735 | 62.070 | 52.859 | 61.472 | 1.00 | 22.47 |
| ATOM | 11733 | CE2 | TYR | B | 735 | 63.789 | 54.421 | 60.900 | 1.00 | 23.07 |
| ATOM | 11734 | CZ | TYR | B | 735 | 63.406 | 53.142 | 61.222 | 1.00 | 22.45 |
| ATOM | 11735 | OH | TYR | B | 735 | 64.365 | 52.155 | 61.340 | 1.00 | 20.77 |
| ATOM | 11736 | N | THR | B | 736 | 57.578 | 56.044 | 58.983 | 1.00 | 21.77 |
| ATOM | 11737 | CA | THR | B | 736 | 56.226 | 56.496 | 58.788 | 1.00 | 22.82 |
| ATOM | 11738 | C | THR | B | 736 | 55.607 | 57.007 | 60.057 | 1.00 | 22.70 |
| ATOM | 11739 | O | THR | B | 736 | 55.587 | 56.294 | 61.042 | 1.00 | 22.68 |
| ATOM | 11740 | CB | THR | B | 736 | 55.368 | 55.322 | 58.241 | 1.00 | 23.25 |
| ATOM | 11741 | OG1 | THR | B | 736 | 55.776 | 55.010 | 56.905 | 1.00 | 22.43 |
| ATOM | 11742 | CG2 | THR | B | 736 | 53.894 | 55.727 | 58.053 | 1.00 | 23.21 |
| ATOM | 11743 | N | ASP | B | 737 | 55.118 | 58.250 | 60.005 | 1.00 | 22.73 |
| ATOM | 11744 | CA | ASP | B | 737 | 54.381 | 58.891 | 61.080 | 1.00 | 23.45 |
| ATOM | 11745 | C | ASP | B | 737 | 55.175 | 59.113 | 62.351 | 1.00 | 23.50 |
| ATOM | 11746 | O | ASP | B | 737 | 54.566 | 59.442 | 63.404 | 1.00 | 22.54 |
| ATOM | 11747 | CB | ASP | B | 737 | 53.109 | 58.086 | 61.456 | 1.00 | 23.34 |
| ATOM | 11748 | CG | ASP | B | 737 | 52.015 | 58.161 | 60.388 | 1.00 | 25.87 |
| ATOM | 11749 | OD1 | ASP | B | 737 | 52.066 | 59.048 | 59.485 | 1.00 | 21.91 |
| ATOM | 11750 | OD2 | ASP | B | 737 | 51.008 | 57.404 | 60.432 | 1.00 | 27.05 |
| ATOM | 11751 | N | GLU | B | 738 | 56.492 | 58.933 | 62.279 | 1.00 | 22.71 |
| ATOM | 11752 | CA | GLU | B | 738 | 57.325 | 59.187 | 63.444 | 1.00 | 23.28 |
| ATOM | 11753 | C | GLU | B | 738 | 57.791 | 60.625 | 63.430 | 1.00 | 22.93 |
| ATOM | 11754 | O | GLU | B | 738 | 57.944 | 61.229 | 62.352 | 1.00 | 21.96 |
| ATOM | 11755 | CB | GLU | B | 738 | 58.519 | 58.243 | 63.512 | 1.00 | 23.38 |
| ATOM | 11756 | CG | GLU | B | 738 | 58.146 | 56.794 | 63.843 | 1.00 | 25.58 |
| ATOM | 11757 | CD | GLU | B | 738 | 57.587 | 56.626 | 65.263 | 1.00 | 29.05 |
| ATOM | 11758 | OE1 | GLU | B | 738 | 58.195 | 57.140 | 66.255 | 1.00 | 27.71 |
| ATOM | 11759 | OE2 | GLU | B | 738 | 56.522 | 55.986 | 65.394 | 1.00 | 33.08 |
| ATOM | 11760 | N | ASP | B | 739 | 58.000 | 61.181 | 64.625 | 1.00 | 22.43 |
| ATOM | 11761 | CA | ASP | B | 739 | 58.505 | 62.562 | 64.722 | 1.00 | 23.17 |
| ATOM | 11762 | C | ASP | B | 739 | 60.036 | 62.685 | 64.963 | 1.00 | 22.55 |
| ATOM | 11763 | O | ASP | B | 739 | 60.756 | 61.738 | 64.656 | 1.00 | 24.43 |
| ATOM | 11764 | CB | ASP | B | 739 | 57.659 | 63.364 | 65.715 | 1.00 | 21.61 |
| ATOM | 11765 | CG | ASP | B | 739 | 57.844 | 62.961 | 67.117 | 1.00 | 20.29 |
| ATOM | 11766 | OD1 | ASP | B | 739 | 58.765 | 62.204 | 67.507 | 1.00 | 20.22 |
| ATOM | 11767 | OD2 | ASP | B | 739 | 57.033 | 63.378 | 67.957 | 1.00 | 21.30 |
| ATOM | 11768 | N | HIS | B | 740 | 60.521 | 63.838 | 65.428 | 1.00 | 22.13 |
| ATOM | 11769 | CA | HIS | B | 740 | 61.938 | 64.043 | 65.752 | 1.00 | 22.65 |
| ATOM | 11770 | C | HIS | B | 740 | 62.535 | 63.036 | 66.723 | 1.00 | 23.57 |
| ATOM | 11771 | O | HIS | B | 740 | 63.739 | 62.760 | 66.661 | 1.00 | 24.38 |
| ATOM | 11772 | CB | HIS | B | 740 | 62.219 | 65.451 | 66.306 | 1.00 | 22.48 |
| ATOM | 11773 | CG | HIS | B | 740 | 63.588 | 65.974 | 65.975 | 1.00 | 21.69 |
| ATOM | 11774 | ND1 | HIS | B | 740 | 64.131 | 65.894 | 64.711 | 1.00 | 24.01 |
| ATOM | 11775 | CD2 | HIS | B | 740 | 64.504 | 66.632 | 66.738 | 1.00 | 21.78 |
| ATOM | 11776 | CE1 | HIS | B | 740 | 65.356 | 66.408 | 64.730 | 1.00 | 23.93 |
| ATOM | 11777 | NE2 | HIS | B | 740 | 65.594 | 66.884 | 65.946 | 1.00 | 21.27 |
| ATOM | 11778 | N | GLY | B | 741 | 61.745 | 62.477 | 67.629 | 1.00 | 24.04 |
| ATOM | 11779 | CA | GLY | B | 741 | 62.324 | 61.524 | 68.557 | 1.00 | 24.01 |
| ATOM | 11780 | C | GLY | B | 741 | 62.381 | 60.115 | 68.020 | 1.00 | 23.93 |
| ATOM | 11781 | O | GLY | B | 741 | 63.160 | 59.294 | 68.501 | 1.00 | 24.35 |
| ATOM | 11782 | N | ILE | B | 742 | 61.616 | 59.840 | 66.975 | 1.00 | 24.05 |
| ATOM | 11783 | CA | ILE | B | 742 | 61.440 | 58.455 | 66.513 | 1.00 | 24.15 |
| ATOM | 11784 | C | ILE | B | 742 | 61.389 | 57.569 | 67.742 | 1.00 | 24.36 |
| ATOM | 11785 | O | ILE | B | 742 | 62.127 | 56.591 | 67.829 | 1.00 | 24.69 |
| ATOM | 11786 | CB | ILE | B | 742 | 62.558 | 57.994 | 65.565 | 1.00 | 24.15 |
| ATOM | 11787 | CG1 | ILE | B | 742 | 62.716 | 58.966 | 64.413 | 1.00 | 24.88 |
| ATOM | 11788 | CG2 | ILE | B | 742 | 62.244 | 56.614 | 64.979 | 1.00 | 22.95 |
| ATOM | 11789 | CD1 | ILE | B | 742 | 63.908 | 58.768 | 63.510 | 1.00 | 25.20 |
| ATOM | 11790 | N | ALA | B | 743 | 60.512 | 57.915 | 68.677 | 1.00 | 24.91 |
| ATOM | 11791 | CA | ALA | B | 743 | 60.485 | 57.274 | 70.001 | 1.00 | 26.61 |
| ATOM | 11792 | C | ALA | B | 743 | 59.398 | 56.284 | 70.291 | 1.00 | 27.02 |
| ATOM | 11793 | O | ALA | B | 743 | 59.448 | 55.645 | 71.345 | 1.00 | 27.69 |
| ATOM | 11794 | CB | ALA | B | 743 | 60.500 | 58.327 | 71.115 | 1.00 | 27.01 |
| ATOM | 11795 | N | SER | B | 744 | 58.441 | 56.084 | 69.393 | 1.00 | 26.67 |
| ATOM | 11796 | CA | SER | B | 744 | 57.478 | 55.042 | 69.692 | 1.00 | 27.68 |
| ATOM | 11797 | C | SER | B | 744 | 58.303 | 53.793 | 69.935 | 1.00 | 28.02 |
| ATOM | 11798 | O | SER | B | 744 | 59.344 | 53.594 | 69.336 | 1.00 | 26.35 |
| ATOM | 11799 | CB | SER | B | 744 | 56.464 | 54.810 | 68.563 | 1.00 | 26.67 |
| ATOM | 11800 | OG | SER | B | 744 | 57.078 | 54.249 | 67.405 | 1.00 | 31.26 |
| ATOM | 11801 | N | SER | B | 745 | 57.784 | 52.919 | 70.771 | 1.00 | 28.75 |
| ATOM | 11802 | CA | SER | B | 745 | 58.516 | 51.748 | 71.161 | 1.00 | 30.40 |
| ATOM | 11803 | C | SER | B | 745 | 59.004 | 50.887 | 69.974 | 1.00 | 29.85 |
| ATOM | 11804 | O | SER | B | 745 | 60.137 | 50.424 | 69.986 | 1.00 | 29.98 |
| ATOM | 11805 | CB | SER | B | 745 | 57.677 | 50.909 | 72.112 | 1.00 | 30.34 |
| ATOM | 11806 | OG | SER | B | 745 | 58.337 | 49.672 | 72.256 | 1.00 | 34.56 |
| ATOM | 11807 | N | THR | B | 746 | 58.182 | 50.675 | 68.946 | 1.00 | 29.65 |
| ATOM | 11808 | CA | THR | B | 746 | 58.630 | 49.790 | 67.847 | 1.00 | 28.90 |
| ATOM | 11809 | C | THR | B | 746 | 59.582 | 50.487 | 66.901 | 1.00 | 27.78 |
| ATOM | 11810 | O | THR | B | 746 | 60.524 | 49.895 | 66.385 | 1.00 | 27.10 |
| ATOM | 11811 | CB | THR | B | 746 | 57.451 | 49.234 | 67.068 | 1.00 | 29.68 |
| ATOM | 11812 | OG1 | THR | B | 746 | 56.540 | 50.295 | 66.707 | 1.00 | 27.85 |
| ATOM | 11813 | CG2 | THR | B | 746 | 56.651 | 48.232 | 67.942 | 1.00 | 30.75 |
| ATOM | 11814 | N | ALA | B | 747 | 59.364 | 51.760 | 66.645 | 1.00 | 27.53 |
| ATOM | 11815 | CA | ALA | B | 747 | 60.298 | 52.413 | 65.734 | 1.00 | 27.18 |
| ATOM | 11816 | C | ALA | B | 747 | 61.675 | 52.509 | 66.400 | 1.00 | 26.49 |
| ATOM | 11817 | O | ALA | B | 747 | 62.724 | 52.320 | 65.745 | 1.00 | 24.33 |
| ATOM | 11818 | CB | ALA | B | 747 | 59.797 | 53.754 | 65.327 | 1.00 | 27.22 |
| ATOM | 11819 | N | HIS | B | 748 | 61.673 | 52.753 | 67.716 | 1.00 | 26.90 |
| ATOM | 11820 | CA | HIS | B | 748 | 62.934 | 52.897 | 68.463 | 1.00 | 26.24 |
| ATOM | 11821 | C | HIS | B | 748 | 63.734 | 51.610 | 68.350 | 1.00 | 27.05 |
| ATOM | 11822 | O | HIS | B | 748 | 64.953 | 51.637 | 68.076 | 1.00 | 25.75 |
| ATOM | 11823 | CB | HIS | B | 748 | 62.669 | 53.226 | 69.931 | 1.00 | 27.44 |
| ATOM | 11824 | CG | HIS | B | 748 | 63.885 | 53.220 | 70.794 | 1.00 | 24.61 |
| ATOM | 11825 | ND1 | HIS | B | 748 | 64.797 | 54.244 | 70.785 | 1.00 | 26.51 |
| ATOM | 11826 | CD2 | HIS | B | 748 | 64.321 | 52.338 | 71.725 | 1.00 | 27.73 |
| ATOM | 11827 | CE1 | HIS | B | 748 | 65.763 | 53.987 | 71.657 | 1.00 | 26.61 |
| ATOM | 11828 | NE2 | HIS | B | 748 | 65.498 | 52.836 | 72.241 | 1.00 | 25.81 |
| ATOM | 11829 | N | GLN | B | 749 | 63.059 | 50.470 | 68.510 | 1.00 | 27.15 |
| ATOM | 11830 | CA | GLN | B | 749 | 63.789 | 49.190 | 68.399 | 1.00 | 28.01 |
| ATOM | 11831 | C | GLN | B | 749 | 64.280 | 48.984 | 66.968 | 1.00 | 27.20 |
| ATOM | 11832 | O | GLN | B | 749 | 65.369 | 48.472 | 66.678 | 1.00 | 26.40 |
| ATOM | 11833 | CB | GLN | B | 749 | 62.896 | 48.020 | 68.842 | 1.00 | 28.88 |
| ATOM | 11834 | CG | GLN | B | 749 | 62.459 | 48.107 | 70.299 | 1.00 | 30.75 |
| ATOM | 11835 | CD | GLN | B | 749 | 61.433 | 47.043 | 70.712 | 1.00 | 35.63 |
| ATOM | 11836 | OE1 | GLN | B | 749 | 61.787 | 45.894 | 70.922 | 1.00 | 41.39 |
| ATOM | 11837 | NE2 | GLN | B | 749 | 60.184 | 47.440 | 70.862 | 1.00 | 37.11 |
| ATOM | 11838 | N | HIS | B | 750 | 63.443 | 49.404 | 66.055 | 1.00 | 27.01 |
| ATOM | 11839 | CA | HIS | B | 750 | 63.721 | 49.167 | 64.681 | 1.00 | 27.01 |
| ATOM | 11840 | C | HIS | B | 750 | 64.890 | 50.052 | 64.227 | 1.00 | 25.80 |
| ATOM | 11841 | O | HIS | B | 750 | 65.794 | 49.546 | 63.602 | 1.00 | 25.66 |
| ATOM | 11842 | CB | HIS | B | 750 | 62.441 | 49.327 | 63.860 | 1.00 | 27.22 |
| ATOM | 11843 | CG | HIS | B | 750 | 62.555 | 48.845 | 62.451 | 1.00 | 28.98 |
| ATOM | 11844 | ND1 | HIS | B | 750 | 61.614 | 48.022 | 61.876 | 1.00 | 34.83 |
| ATOM | 11845 | CD2 | HIS | B | 750 | 63.489 | 49.055 | 61.506 | 1.00 | 28.49 |
| ATOM | 11846 | CE1 | HIS | B | 750 | 61.968 | 47.748 | 60.635 | 1.00 | 31.78 |
| ATOM | 11847 | NE2 | HIS | B | 750 | 63.099 | 48.366 | 60.383 | 1.00 | 27.98 |
| ATOM | 11848 | N | ILE | B | 751 | 64.915 | 51.331 | 64.584 | 1.00 | 24.75 |
| ATOM | 11849 | CA | ILE | B | 751 | 65.981 | 52.202 | 64.090 | 1.00 | 24.56 |
| ATOM | 11850 | C | ILE | B | 751 | 67.350 | 51.745 | 64.630 | 1.00 | 24.17 |
| ATOM | 11851 | O | ILE | B | 751 | 68.305 | 51.572 | 63.858 | 1.00 | 23.48 |
| ATOM | 11852 | CB | ILE | B | 751 | 65.711 | 53.688 | 64.305 | 1.00 | 23.55 |
| ATOM | 11853 | CG1 | ILE | B | 751 | 66.729 | 54.493 | 63.524 | 1.00 | 25.42 |
| ATOM | 11854 | CG2 | ILE | B | 751 | 65.814 | 54.070 | 65.770 | 1.00 | 26.58 |
| ATOM | 11855 | CD1 | ILE | B | 751 | 66.537 | 55.999 | 63.550 | 1.00 | 25.36 |
| ATOM | 11856 | N | TYR | B | 752 | 67.435 | 51.461 | 65.922 | 1.00 | 23.88 |
| ATOM | 11857 | CA | TYR | B | 752 | 68.716 | 51.006 | 66.471 | 1.00 | 23.89 |
| ATOM | 11858 | C | TYR | B | 752 | 69.152 | 49.642 | 65.939 | 1.00 | 24.36 |
| ATOM | 11859 | O | TYR | B | 752 | 70.356 | 49.341 | 65.864 | 1.00 | 24.86 |
| ATOM | 11860 | CB | TYR | B | 752 | 68.696 | 51.033 | 68.016 | 1.00 | 23.90 |
| ATOM | 11861 | CG | TYR | B | 752 | 68.908 | 52.438 | 68.505 | 1.00 | 23.39 |
| ATOM | 11862 | CD1 | TYR | B | 752 | 70.202 | 52.985 | 68.564 | 1.00 | 21.30 |
| ATOM | 11863 | CD2 | TYR | B | 752 | 67.820 | 53.256 | 68.841 | 1.00 | 21.53 |
| ATOM | 11864 | CE1 | TYR | B | 752 | 70.406 | 54.301 | 69.001 | 1.00 | 19.82 |
| ATOM | 11865 | CE2 | TYR | B | 752 | 68.023 | 54.584 | 69.216 | 1.00 | 22.84 |
| ATOM | 11866 | CZ | TYR | B | 752 | 69.322 | 55.069 | 69.328 | 1.00 | 22.05 |
| ATOM | 11867 | OH | TYR | B | 752 | 69.504 | 56.353 | 69.701 | 1.00 | 26.93 |
| ATOM | 11868 | N | THR | B | 753 | 68.190 | 48.823 | 65.545 | 1.00 | 24.72 |
| ATOM | 11869 | CA | THR | B | 753 | 68.499 | 47.540 | 64.949 | 1.00 | 25.04 |
| ATOM | 11870 | C | THR | B | 753 | 69.130 | 47.719 | 63.599 | 1.00 | 25.21 |
| ATOM | 11871 | O | THR | B | 753 | 70.055 | 46.986 | 63.227 | 1.00 | 25.42 |
| ATOM | 11872 | CB | THR | B | 753 | 67.254 | 46.686 | 64.821 | 1.00 | 25.19 |
| ATOM | 11873 | OG1 | THR | B | 753 | 66.815 | 46.327 | 66.129 | 1.00 | 26.51 |
| ATOM | 11874 | CG2 | THR | B | 753 | 67.552 | 45.322 | 64.189 | 1.00 | 26.77 |
| ATOM | 11875 | N | HIS | B | 754 | 68.591 | 48.676 | 62.859 | 1.00 | 25.43 |
| ATOM | 11876 | CA | HIS | B | 754 | 68.992 | 48.934 | 61.509 | 1.00 | 24.78 |
| ATOM | 11877 | C | HIS | B | 754 | 70.363 | 49.549 | 61.526 | 1.00 | 24.51 |
| ATOM | 11878 | O | HIS | B | 754 | 71.207 | 49.166 | 60.769 | 1.00 | 23.52 |
| ATOM | 11879 | CB | HIS | B | 754 | 68.031 | 49.899 | 60.832 | 1.00 | 24.32 |
| ATOM | 11880 | CG | HIS | B | 754 | 68.090 | 49.863 | 59.336 | 1.00 | 23.90 |
| ATOM | 11881 | ND1 | HIS | B | 754 | 67.869 | 48.707 | 58.609 | 1.00 | 23.25 |
| ATOM | 11882 | CD2 | HIS | B | 754 | 68.269 | 50.852 | 58.428 | 1.00 | 24.23 |
| ATOM | 11883 | CE1 | HIS | B | 754 | 67.960 | 48.982 | 57.319 | 1.00 | 21.88 |
| ATOM | 11884 | NE2 | HIS | B | 754 | 68.190 | 50.276 | 57.180 | 1.00 | 23.11 |
| ATOM | 11885 | N | MET | B | 755 | 70.577 | 50.512 | 62.407 | 1.00 | 25.06 |
| ATOM | 11886 | CA | MET | B | 755 | 71.887 | 51.163 | 62.511 | 1.00 | 24.45 |
| ATOM | 11887 | C | MET | B | 755 | 72.973 | 50.195 | 63.052 | 1.00 | 24.60 |
| ATOM | 11888 | O | MET | B | 755 | 74.151 | 50.335 | 62.717 | 1.00 | 22.03 |
| ATOM | 11889 | CB | MET | B | 755 | 71.789 | 52.381 | 63.426 | 1.00 | 24.41 |
| ATOM | 11890 | CG | MET | B | 755 | 70.852 | 53.499 | 62.924 | 1.00 | 24.99 |
| ATOM | 11891 | SD | MET | B | 755 | 71.094 | 55.090 | 63.747 | 1.00 | 28.21 |
| ATOM | 11892 | CE | MET | B | 755 | 70.546 | 54.690 | 65.314 | 1.00 | 29.16 |
| ATOM | 11893 | N | SER | B | 756 | 72.583 | 49.243 | 63.909 | 1.00 | 24.26 |
| ATOM | 11894 | CA | SER | B | 756 | 73.553 | 48.292 | 64.433 | 1.00 | 24.91 |
| ATOM | 11895 | C | SER | B | 756 | 74.000 | 47.471 | 63.221 | 1.00 | 26.55 |
| ATOM | 11896 | O | SER | B | 756 | 75.194 | 47.388 | 62.952 | 1.00 | 26.29 |
| ATOM | 11897 | CB | SER | B | 756 | 73.006 | 47.385 | 65.550 | 1.00 | 24.53 |
| ATOM | 11898 | OG | SER | B | 756 | 72.423 | 48.114 | 66.652 | 1.00 | 23.99 |
| ATOM | 11899 | N | HIS | B | 757 | 73.045 | 46.956 | 62.430 | 1.00 | 27.68 |
| ATOM | 11900 | CA | HIS | B | 757 | 73.434 | 46.220 | 61.197 | 1.00 | 28.64 |
| ATOM | 11901 | C | HIS | B | 757 | 74.393 | 47.051 | 60.329 | 1.00 | 28.24 |
| ATOM | 11902 | O | HIS | B | 757 | 75.466 | 46.568 | 59.873 | 1.00 | 28.92 |
| ATOM | 11903 | CB | HIS | B | 757 | 72.213 | 45.782 | 60.351 | 1.00 | 28.51 |
| ATOM | 11904 | CG | HIS | B | 757 | 71.382 | 44.709 | 60.993 | 1.00 | 30.88 |
| ATOM | 11905 | ND1 | HIS | B | 757 | 71.936 | 43.626 | 61.646 | 1.00 | 37.16 |
| ATOM | 11906 | CD2 | HIS | B | 757 | 70.037 | 44.550 | 61.083 | 1.00 | 31.61 |
| ATOM | 11907 | CE1 | HIS | B | 757 | 70.966 | 42.849 | 62.109 | 1.00 | 35.73 |
| ATOM | 11908 | NE2 | HIS | B | 757 | 69.806 | 43.388 | 61.784 | 1.00 | 31.22 |
| ATOM | 11909 | N | PHE | B | 758 | 74.017 | 48.286 | 60.065 | 1.00 | 26.88 |
| ATOM | 11910 | CA | PHE | B | 758 | 74.846 | 49.117 | 59.214 | 1.00 | 27.31 |
| ATOM | 11911 | C | PHE | B | 758 | 76.286 | 49.250 | 59.739 | 1.00 | 28.46 |
| ATOM | 11912 | O | PHE | B | 758 | 77.293 | 49.055 | 59.003 | 1.00 | 28.22 |
| ATOM | 11913 | CB | PHE | B | 758 | 74.221 | 50.499 | 59.123 | 1.00 | 27.19 |
| ATOM | 11914 | CG | PHE | B | 758 | 74.922 | 51.419 | 58.206 | 1.00 | 26.78 |
| ATOM | 11915 | CD1 | PHE | B | 758 | 74.714 | 51.341 | 56.829 | 1.00 | 27.43 |
| ATOM | 11916 | CD2 | PHE | B | 758 | 75.776 | 52.367 | 58.697 | 1.00 | 27.39 |
| ATOM | 11917 | CE1 | PHE | B | 758 | 75.372 | 52.179 | 55.967 | 1.00 | 27.51 |
| ATOM | 11918 | CE2 | PHE | B | 758 | 76.428 | 53.234 | 57.847 | 1.00 | 28.75 |
| ATOM | 11919 | CZ | PHE | B | 758 | 76.235 | 53.140 | 56.465 | 1.00 | 28.75 |
| ATOM | 11920 | N | ILE | B | 759 | 76.404 | 49.597 | 61.009 | 1.00 | 28.83 |
| ATOM | 11921 | CA | ILE | B | 759 | 77.723 | 49.762 | 61.582 | 1.00 | 30.09 |
| ATOM | 11922 | C | ILE | B | 759 | 78.505 | 48.449 | 61.646 | 1.00 | 30.59 |
| ATOM | 11923 | O | ILE | B | 759 | 79.684 | 48.447 | 61.372 | 1.00 | 30.39 |
| ATOM | 11924 | CB | ILE | B | 759 | 77.631 | 50.512 | 62.924 | 1.00 | 30.25 |
| ATOM | 11925 | CG1 | ILE | B | 759 | 77.290 | 51.949 | 62.602 | 1.00 | 32.97 |
| ATOM | 11926 | CG2 | ILE | B | 759 | 78.982 | 50.584 | 63.638 | 1.00 | 30.73 |
| ATOM | 11927 | CD1 | ILE | B | 759 | 76.802 | 52.655 | 63.707 | 1.00 | 36.65 |
| ATOM | 11928 | N | LYS | B | 760 | 77.856 | 47.333 | 61.927 | 1.00 | 32.18 |
| ATOM | 11929 | CA | LYS | B | 760 | 78.582 | 46.073 | 62.000 | 1.00 | 34.39 |
| ATOM | 11930 | C | LYS | B | 760 | 79.181 | 45.764 | 60.636 | 1.00 | 35.25 |
| ATOM | 11931 | O | LYS | B | 760 | 80.369 | 45.391 | 60.507 | 1.00 | 35.22 |
| ATOM | 11932 | CB | LYS | B | 760 | 77.679 | 44.917 | 62.459 | 1.00 | 34.88 |
| ATOM | 11933 | CG | LYS | B | 760 | 77.221 | 44.989 | 63.895 | 1.00 | 36.76 |
| ATOM | 11934 | CD | LYS | B | 760 | 77.344 | 43.653 | 64.570 | 1.00 | 39.28 |
| ATOM | 11935 | CE | LYS | B | 760 | 76.063 | 42.919 | 64.666 | 1.00 | 42.26 |
| ATOM | 11936 | NZ | LYS | B | 760 | 76.310 | 41.567 | 65.259 | 1.00 | 45.49 |
| ATOM | 11937 | N | GLN | B | 761 | 78.377 | 45.970 | 59.604 | 1.00 | 35.65 |
| ATOM | 11938 | CA | GLN | B | 761 | 78.845 | 45.690 | 58.254 | 1.00 | 37.11 |
| ATOM | 11939 | C | GLN | B | 761 | 79.935 | 46.657 | 57.813 | 1.00 | 37.13 |
| ATOM | 11940 | O | GLN | B | 761 | 80.897 | 46.238 | 57.191 | 1.00 | 36.55 |
| ATOM | 11941 | CB | GLN | B | 761 | 77.687 | 45.622 | 57.237 | 1.00 | 37.51 |
| ATOM | 11942 | CG | GLN | B | 761 | 76.992 | 46.909 | 56.935 | 1.00 | 40.25 |
| ATOM | 11943 | CD | GLN | B | 761 | 77.690 | 47.748 | 55.884 | 1.00 | 46.19 |
| ATOM | 11944 | OE1 | GLN | B | 761 | 78.493 | 47.228 | 55.088 | 1.00 | 48.05 |
| ATOM | 11945 | NE2 | GLN | B | 761 | 77.396 | 49.061 | 55.880 | 1.00 | 47.46 |
| ATOM | 11946 | N | CYS | B | 762 | 79.810 | 47.934 | 58.161 | 1.00 | 37.50 |
| ATOM | 11947 | CA | CYS | B | 762 | 80.856 | 48.893 | 57.840 | 1.00 | 38.43 |
| ATOM | 11948 | C | CYS | B | 762 | 82.187 | 48.452 | 58.431 | 1.00 | 38.75 |
| ATOM | 11949 | O | CYS | B | 762 | 83.237 | 48.656 | 57.837 | 1.00 | 38.74 |
| ATOM | 11950 | CB | CYS | B | 762 | 80.510 | 50.249 | 58.433 | 1.00 | 38.71 |
| ATOM | 11951 | SG | CYS | B | 762 | 81.673 | 51.592 | 58.105 | 1.00 | 41.98 |
| ATOM | 11952 | N | PHE | B | 763 | 82.118 | 47.832 | 59.600 | 1.00 | 38.96 |
| ATOM | 11953 | CA | PHE | B | 763 | 83.287 | 47.495 | 60.381 | 1.00 | 39.41 |
| ATOM | 11954 | C | PHE | B | 763 | 83.680 | 46.005 | 60.240 | 1.00 | 40.32 |
| ATOM | 11955 | O | PHE | B | 763 | 84.482 | 45.487 | 61.015 | 1.00 | 39.92 |
| ATOM | 11956 | CB | PHE | B | 763 | 82.956 | 47.807 | 61.868 | 1.00 | 38.92 |
| ATOM | 11957 | CG | PHE | B | 763 | 83.038 | 49.273 | 62.249 | 1.00 | 35.95 |
| ATOM | 11958 | CD1 | PHE | B | 763 | 83.606 | 50.211 | 61.423 | 1.00 | 34.28 |
| ATOM | 11959 | CD2 | PHE | B | 763 | 82.584 | 49.692 | 63.488 | 1.00 | 36.76 |
| ATOM | 11960 | CE1 | PHE | B | 763 | 83.702 | 51.517 | 61.819 | 1.00 | 33.55 |
| ATOM | 11961 | CE2 | PHE | B | 763 | 82.673 | 51.020 | 63.878 | 1.00 | 33.00 |
| ATOM | 11962 | CZ | PHE | B | 763 | 83.227 | 51.917 | 63.061 | 1.00 | 32.46 |
| ATOM | 11963 | N | SER | B | 764 | 83.060 | 45.320 | 59.283 | 1.00 | 41.75 |
| ATOM | 11964 | CA | SER | B | 764 | 83.281 | 43.895 | 59.036 | 1.00 | 42.70 |
| ATOM | 11965 | C | SER | B | 764 | 83.192 | 43.024 | 60.284 | 1.00 | 44.33 |
| ATOM | 11966 | O | SER | B | 764 | 84.038 | 42.145 | 60.498 | 1.00 | 45.27 |
| ATOM | 11967 | CB | SER | B | 764 | 84.631 | 43.677 | 58.354 | 1.00 | 42.68 |
| ATOM | 11968 | OG | SER | B | 764 | 84.761 | 44.575 | 57.279 | 1.00 | 40.08 |
| ATOM | 11969 | N | LEU | B | 765 | 82.167 | 43.263 | 61.091 | 1.00 | 45.12 |
| ATOM | 11970 | CA | LEU | B | 765 | 81.891 | 42.461 | 62.264 | 1.00 | 46.20 |
| ATOM | 11971 | C | LEU | B | 765 | 80.703 | 41.527 | 61.982 | 1.00 | 47.52 |
| ATOM | 11972 | O | LEU | B | 765 | 79.635 | 42.000 | 61.626 | 1.00 | 47.72 |
| ATOM | 11973 | CB | LEU | B | 765 | 81.550 | 43.373 | 63.450 | 1.00 | 46.04 |
| ATOM | 11974 | CG | LEU | B | 765 | 82.688 | 44.265 | 63.995 | 1.00 | 46.16 |
| ATOM | 11975 | CD1 | LEU | B | 765 | 82.184 | 45.259 | 64.996 | 1.00 | 46.29 |
| ATOM | 11976 | CD2 | LEU | B | 765 | 83.787 | 43.424 | 64.619 | 1.00 | 46.53 |
| ATOM | 11977 | N | PRO | B | 766 | 80.873 | 40.212 | 62.143 | 1.00 | 48.72 |
| ATOM | 11978 | CA | PRO | B | 766 | 79.765 | 39.261 | 61.948 | 1.00 | 49.30 |
| ATOM | 11979 | C | PRO | B | 766 | 78.590 | 39.490 | 62.907 | 1.00 | 49.42 |
| ATOM | 11980 | O | PRO | B | 766 | 78.756 | 39.282 | 64.104 | 1.00 | 49.18 |
| ATOM | 11981 | CB | PRO | B | 766 | 80.407 | 37.899 | 62.243 | 1.00 | 49.19 |
| ATOM | 11982 | CG | PRO | B | 766 | 81.831 | 38.101 | 62.017 | 1.00 | 49.63 |
| ATOM | 11983 | CD | PRO | B | 766 | 82.124 | 39.517 | 62.486 | 1.00 | 49.18 |
| TER | 11984 | | PRO | B | 766 | | | | | |
| HETATM | 11985 | C1 | NAG | B | 793 | 33.999 | 64.461 | 78.683 | 1.00 | 57.99 |
| HETATM | 11986 | C2 | NAG | B | 793 | 32.573 | 65.000 | 78.742 | 1.00 | 61.12 |
| HETATM | 11987 | N2 | NAG | B | 793 | 32.577 | 66.287 | 79.402 | 1.00 | 61.79 |
| HETATM | 11988 | C7 | NAG | B | 793 | 33.128 | 66.514 | 80.598 | 1.00 | 63.53 |
| HETATM | 11989 | O7 | NAG | B | 793 | 33.450 | 67.654 | 80.963 | 1.00 | 65.37 |
| HETATM | 11990 | C8 | NAG | B | 793 | 33.376 | 65.370 | 81.538 | 1.00 | 63.95 |
| HETATM | 11991 | C3 | NAG | B | 793 | 31.628 | 63.988 | 79.397 | 1.00 | 63.28 |
| HETATM | 11992 | O3 | NAG | B | 793 | 30.285 | 64.421 | 79.591 | 1.00 | 61.99 |
| HETATM | 11993 | C4 | NAG | B | 793 | 31.723 | 62.749 | 78.530 | 1.00 | 63.93 |
| HETATM | 11994 | O4 | NAG | B | 793 | 30.728 | 61.837 | 78.927 | 1.00 | 62.43 |
| HETATM | 11995 | C5 | NAG | B | 793 | 33.122 | 62.160 | 78.732 | 1.00 | 64.92 |
| HETATM | 11996 | C6 | NAG | B | 793 | 33.290 | 60.932 | 77.842 | 1.00 | 66.07 |
| HETATM | 11997 | O6 | NAG | B | 793 | 34.269 | 61.217 | 76.867 | 1.00 | 66.84 |
| HETATM | 11998 | O5 | NAG | B | 793 | 34.179 | 63.069 | 78.445 | 1.00 | 60.71 |
| HETATM | 11999 | C1 | NAG | B | 794 | 55.667 | 62.444 | 110.980 | 1.00 | 34.19 |
| HETATM | 12000 | C2 | NAG | B | 794 | 54.300 | 62.821 | 110.434 | 1.00 | 34.04 |
| HETATM | 12001 | N2 | NAG | B | 794 | 54.500 | 63.199 | 109.054 | 1.00 | 32.45 |
| HETATM | 12002 | C7 | NAG | B | 794 | 54.026 | 62.522 | 108.007 | 1.00 | 30.69 |
| HETATM | 12003 | O7 | NAG | B | 794 | 53.459 | 61.412 | 108.025 | 1.00 | 29.04 |
| HETATM | 12004 | C8 | NAG | B | 794 | 54.215 | 63.246 | 106.719 | 1.00 | 28.12 |
| HETATM | 12005 | C3 | NAG | B | 794 | 53.760 | 64.012 | 111.199 | 1.00 | 35.55 |
| HETATM | 12006 | O3 | NAG | B | 794 | 52.487 | 64.340 | 110.702 | 1.00 | 40.00 |
| HETATM | 12007 | C4 | NAG | B | 794 | 53.648 | 63.653 | 112.679 | 1.00 | 36.40 |
| HETATM | 12008 | O4 | NAG | B | 794 | 53.272 | 64.805 | 113.391 | 1.00 | 34.43 |
| HETATM | 12009 | C5 | NAG | B | 794 | 55.010 | 63.195 | 113.177 | 1.00 | 32.95 |
| HETATM | 12010 | C6 | NAG | B | 794 | 54.946 | 62.678 | 114.607 | 1.00 | 33.12 |
| HETATM | 12011 | O6 | NAG | B | 794 | 53.967 | 61.672 | 114.764 | 1.00 | 28.48 |
| HETATM | 12012 | O5 | NAG | B | 794 | 55.494 | 62.146 | 112.353 | 1.00 | 35.51 |
| HETATM | 12013 | C1 | NAG | B | 796 | 46.134 | 89.074 | 64.573 | 1.00 | 37.06 |
| HETATM | 12014 | C2 | NAG | B | 796 | 45.064 | 90.053 | 65.068 | 1.00 | 39.18 |
| HETATM | 12015 | N2 | NAG | B | 796 | 44.194 | 89.535 | 66.099 | 1.00 | 38.69 |
| HETATM | 12016 | C7 | NAG | B | 796 | 44.254 | 89.894 | 67.359 | 1.00 | 41.45 |
| HETATM | 12017 | O7 | NAG | B | 796 | 45.136 | 90.611 | 67.793 | 1.00 | 45.89 |
| HETATM | 12018 | C8 | NAG | B | 796 | 43.179 | 89.381 | 68.270 | 1.00 | 41.70 |
| HETATM | 12019 | C3 | NAG | B | 796 | 44.162 | 90.454 | 63.915 | 1.00 | 40.21 |
| HETATM | 12020 | O3 | NAG | B | 796 | 43.318 | 91.468 | 64.403 | 1.00 | 39.86 |
| HETATM | 12021 | C4 | NAG | B | 796 | 44.987 | 90.975 | 62.748 | 1.00 | 43.50 |
| HETATM | 12022 | O4 | NAG | B | 796 | 44.302 | 90.814 | 61.526 | 1.00 | 44.51 |
| HETATM | 12023 | C5 | NAG | B | 796 | 46.322 | 90.248 | 62.597 | 1.00 | 43.28 |
| HETATM | 12024 | C6 | NAG | B | 796 | 47.244 | 91.170 | 61.831 | 1.00 | 43.03 |
| HETATM | 12025 | O6 | NAG | B | 796 | 47.617 | 90.355 | 60.756 | 1.00 | 44.68 |
| HETATM | 12026 | O5 | NAG | B | 796 | 46.961 | 89.876 | 63.806 | 1.00 | 36.35 |
| HETATM | 12027 | C1 | NAG | B | 797 | 49.268 | 49.936 | 96.936 | 1.00 | 61.01 |
| HETATM | 12028 | C2 | NAG | B | 797 | 49.691 | 48.602 | 96.293 | 1.00 | 62.31 |
| HETATM | 12029 | N2 | NAG | B | 797 | 48.733 | 48.188 | 95.291 | 1.00 | 62.72 |
| HETATM | 12030 | C7 | NAG | B | 797 | 48.798 | 48.715 | 94.079 | 1.00 | 63.61 |
| HETATM | 12031 | O7 | NAG | B | 797 | 49.868 | 48.995 | 93.512 | 1.00 | 61.06 |
| HETATM | 12032 | C8 | NAG | B | 797 | 47.463 | 48.951 | 93.439 | 1.00 | 64.40 |
| HETATM | 12033 | C3 | NAG | B | 797 | 49.905 | 47.435 | 97.253 | 1.00 | 61.57 |
| HETATM | 12034 | O3 | NAG | B | 797 | 50.543 | 46.394 | 96.566 | 1.00 | 60.47 |
| HETATM | 12035 | C4 | NAG | B | 797 | 50.776 | 47.922 | 98.392 | 1.00 | 61.34 |
| HETATM | 12036 | O4 | NAG | B | 797 | 51.162 | 46.853 | 99.231 | 1.00 | 59.75 |
| HETATM | 12037 | C5 | NAG | B | 797 | 49.921 | 48.982 | 99.073 | 1.00 | 61.24 |
| HETATM | 12038 | C6 | NAG | B | 797 | 50.437 | 49.402 | 100.454 | 1.00 | 60.99 |
| HETATM | 12039 | O6 | NAG | B | 797 | 51.831 | 49.550 | 100.437 | 1.00 | 58.34 |
| HETATM | 12040 | O5 | NAG | B | 797 | 49.878 | 50.102 | 98.209 | 1.00 | 61.78 |
| HETATM | 12041 | O | HOH | | 1 | 69.755 | 80.399 | 86.643 | 1.00 | 7.73 |
| HETATM | 12042 | O | HOH | | 2 | 39.998 | 46.901 | 18.457 | 1.00 | 10.31 |
| HETATM | 12043 | O | HOH | | 4 | 62.220 | 40.486 | 27.246 | 1.00 | 12.87 |
| HETATM | 12044 | O | HOH | | 5 | 64.826 | 62.571 | 43.828 | 1.00 | 19.31 |
| HETATM | 12045 | O | HOH | | 6 | 56.767 | 36.556 | 36.335 | 1.00 | 21.00 |
| HETATM | 12046 | O | HOH | | 7 | 50.245 | 35.394 | 15.711 | 1.00 | 20.14 |
| HETATM | 12047 | O | HOH | | 8 | 78.281 | 56.483 | 80.453 | 1.00 | 37.18 |
| HETATM | 12048 | O | HOH | | 9 | 56.125 | 73.117 | 69.619 | 1.00 | 19.38 |
| HETATM | 12049 | O | HOH | | 10 | 36.083 | 37.083 | 46.767 | 1.00 | 39.78 |
| HETATM | 12050 | O | HOH | | 11 | 59.581 | 32.583 | 52.936 | 1.00 | 27.38 |
| HETATM | 12051 | O | HOH | | 12 | 34.041 | 50.937 | 39.008 | 1.00 | 20.61 |
| HETATM | 12052 | O | HOH | | 13 | 65.320 | 47.187 | 62.009 | 1.00 | 24.23 |
| HETATM | 12053 | O | HOH | | 14 | 31.494 | 58.838 | 35.693 | 1.00 | 22.60 |
| HETATM | 12054 | O | HOH | | 15 | 62.642 | 72.239 | 59.850 | 1.00 | 23.18 |
| HETATM | 12055 | O | HOH | | 16 | 77.995 | 58.866 | 78.221 | 1.00 | 15.42 |
| HETATM | 12056 | O | HOH | | 17 | 58.533 | 67.745 | 55.662 | 1.00 | 22.30 |
| HETATM | 12057 | O | HOH | | 18 | 88.208 | 29.565 | 21.093 | 1.00 | 49.26 |
| HETATM | 12058 | O | HOH | | 19 | 40.525 | 72.824 | 73.221 | 1.00 | 21.32 |
| HETATM | 12059 | O | HOH | | 20 | 48.198 | 59.827 | 34.355 | 1.00 | 25.55 |
| HETATM | 12060 | O | HOH | | 21 | 73.384 | 76.365 | 48.426 | 1.00 | 33.50 |
| HETATM | 12061 | O | HOH | | 22 | 50.583 | 31.754 | 30.128 | 1.00 | 27.05 |
| HETATM | 12062 | O | HOH | | 23 | 31.043 | 56.428 | 38.581 | 1.00 | 23.41 |
| HETATM | 12063 | O | HOH | | 24 | 52.509 | 59.981 | 46.771 | 1.00 | 25.42 |
| HETATM | 12064 | O | HOH | | 25 | 36.878 | 45.981 | 19.156 | 1.00 | 26.52 |
| HETATM | 12065 | O | HOH | | 26 | 65.794 | 61.426 | 46.509 | 1.00 | 22.37 |
| HETATM | 12066 | O | HOH | | 27 | 50.084 | 61.646 | 50.977 | 1.00 | 25.11 |
| HETATM | 12067 | O | HOH | | 28 | 54.775 | 55.861 | 63.543 | 1.00 | 31.76 |
| HETATM | 12068 | O | HOH | | 29 | 80.149 | 72.731 | 69.030 | 1.00 | 22.40 |
| HETATM | 12069 | O | HOH | | 30 | 27.782 | 60.784 | 35.598 | 1.00 | 29.54 |
| HETATM | 12070 | O | HOH | | 31 | 74.208 | 56.149 | 72.170 | 1.00 | 19.00 |
| HETATM | 12071 | O | HOH | | 32 | 82.869 | 57.914 | 96.204 | 1.00 | 32.13 |
| HETATM | 12072 | O | HOH | | 33 | 80.923 | 58.743 | 73.558 | 1.00 | 27.31 |
| HETATM | 12073 | O | HOH | | 34 | 55.022 | 78.846 | 65.695 | 1.00 | 26.47 |
| HETATM | 12074 | O | HOH | | 35 | 69.840 | 71.834 | 89.800 | 1.00 | 22.74 |
| HETATM | 12075 | O | HOH | | 36 | 30.001 | 58.932 | 39.011 | 1.00 | 25.83 |
| HETATM | 12076 | O | HOH | | 37 | 62.208 | 61.143 | 103.510 | 1.00 | 37.15 |
| HETATM | 12077 | O | HOH | | 38 | 57.842 | 52.910 | 58.684 | 1.00 | 23.27 |
| HETATM | 12078 | O | HOH | | 39 | 37.983 | 42.872 | 24.658 | 1.00 | 20.62 |
| HETATM | 12079 | O | HOH | | 40 | 52.081 | 75.115 | 70.879 | 1.00 | 26.06 |
| HETATM | 12080 | O | HOH | | 41 | 72.496 | 54.302 | 43.326 | 1.00 | 32.87 |
| HETATM | 12081 | O | HOH | | 42 | 73.302 | 69.745 | 86.804 | 1.00 | 26.45 |
| HETATM | 12082 | O | HOH | | 43 | 43.663 | 65.019 | 34.139 | 1.00 | 23.93 |
| HETATM | 12083 | O | HOH | | 44 | 66.893 | 48.699 | 89.087 | 1.00 | 30.37 |
| HETATM | 12084 | O | HOH | | 45 | 56.462 | 68.617 | 53.861 | 1.00 | 24.87 |
| HETATM | 12085 | O | HOH | | 46 | 58.082 | 66.385 | 58.226 | 1.00 | 23.90 |
| HETATM | 12086 | O | HOH | | 48 | 25.975 | 61.038 | 32.629 | 1.00 | 27.79 |
| HETATM | 12087 | O | HOH | | 49 | 34.089 | 59.708 | 48.959 | 1.00 | 28.45 |
| HETATM | 12088 | O | HOH | | 50 | 82.210 | 91.526 | 102.461 | 1.00 | 41.43 |
| HETATM | 12089 | O | HOH | | 51 | 29.874 | 69.711 | 84.012 | 1.00 | 35.20 |
| HETATM | 12090 | O | HOH | | 52 | 83.150 | 70.002 | 67.331 | 1.00 | 33.57 |
| HETATM | 12091 | O | HOH | | 53 | 43.509 | 44.094 | 56.593 | 1.00 | 32.63 |
| HETATM | 12092 | O | HOH | | 54 | 38.606 | 76.598 | 91.780 | 1.00 | 30.60 |
| HETATM | 12093 | O | HOH | | 55 | 51.005 | 36.317 | 35.795 | 1.00 | 39.38 |
| HETATM | 12094 | O | HOH | | 56 | 70.432 | 83.664 | 85.676 | 1.00 | 26.32 |
| HETATM | 12095 | O | HOH | | 57 | 35.211 | 69.922 | 74.499 | 1.00 | 37.11 |
| HETATM | 12096 | O | HOH | | 58 | 81.995 | 70.997 | 89.633 | 1.00 | 28.50 |
| HETATM | 12097 | O | HOH | | 59 | 50.463 | 38.807 | 22.087 | 1.00 | 39.80 |
| HETATM | 12098 | O | HOH | | 60 | 47.949 | 31.885 | 21.015 | 1.00 | 22.69 |
| HETATM | 12099 | O | HOH | | 61 | 47.182 | 54.781 | 45.141 | 1.00 | 20.23 |
| HETATM | 12100 | O | HOH | | 62 | 60.983 | 33.345 | 50.537 | 1.00 | 28.58 |
| HETATM | 12101 | O | HOH | | 63 | 65.450 | 85.673 | 65.652 | 1.00 | 29.90 |
| HETATM | 12102 | O | HOH | | 64 | 62.565 | 62.304 | 62.410 | 1.00 | 16.92 |
| HETATM | 12103 | O | HOH | | 65 | 61.679 | 72.418 | 49.807 | 1.00 | 24.32 |
| HETATM | 12104 | O | HOH | | 66 | 79.506 | 66.115 | 69.527 | 1.00 | 26.96 |
| HETATM | 12105 | O | HOH | | 68 | 54.535 | 64.150 | 64.525 | 1.00 | 19.13 |
| HETATM | 12106 | O | HOH | | 69 | 60.608 | 52.351 | 58.371 | 1.00 | 22.94 |
| HETATM | 12107 | O | HOH | | 70 | 51.046 | 59.344 | 49.440 | 1.00 | 22.26 |
| HETATM | 12108 | O | HOH | | 72 | 56.903 | 55.928 | 42.618 | 1.00 | 21.85 |
| HETATM | 12109 | O | HOH | | 73 | 50.482 | 56.281 | 62.640 | 1.00 | 24.14 |
| HETATM | 12110 | O | HOH | | 74 | 83.719 | 69.898 | 87.710 | 1.00 | 24.93 |
| HETATM | 12111 | O | HOH | | 75 | 79.910 | 80.755 | 77.636 | 1.00 | 24.86 |
| HETATM | 12112 | O | HOH | | 76 | 57.730 | 71.048 | 70.318 | 1.00 | 22.20 |
| HETATM | 12113 | O | HOH | | 77 | 65.844 | 87.314 | 99.634 | 1.00 | 23.32 |
| HETATM | 12114 | O | HOH | | 78 | 73.533 | 63.176 | 68.120 | 1.00 | 17.04 |
| HETATM | 12115 | O | HOH | | 79 | 82.010 | 77.243 | 76.246 | 1.00 | 23.87 |
| HETATM | 12116 | O | HOH | | 80 | 57.970 | 68.804 | 68.829 | 1.00 | 19.26 |
| HETATM | 12117 | O | HOH | | 81 | 81.575 | 64.030 | 69.005 | 1.00 | 27.93 |
| HETATM | 12118 | O | HOH | | 82 | 64.683 | 44.537 | 33.113 | 1.00 | 24.42 |
| HETATM | 12119 | O | HOH | | 83 | 46.606 | 55.961 | 55.295 | 1.00 | 22.83 |
| HETATM | 12120 | O | HOH | | 84 | 52.899 | 58.974 | 38.346 | 1.00 | 18.29 |
| HETATM | 12121 | O | HOH | | 85 | 73.770 | 82.389 | 80.349 | 1.00 | 21.67 |
| HETATM | 12122 | O | HOH | | 86 | 44.010 | 34.967 | 29.064 | 1.00 | 26.14 |
| HETATM | 12123 | O | HOH | | 87 | 58.796 | 62.203 | 38.296 | 1.00 | 38.76 |
| HETATM | 12124 | O | HOH | | 88 | 52.117 | 52.744 | 34.980 | 1.00 | 34.59 |
| HETATM | 12125 | O | HOH | | 89 | 57.055 | 44.447 | 37.217 | 1.00 | 20.82 |
| HETATM | 12126 | O | HOH | | 90 | 51.256 | 34.490 | 38.049 | 1.00 | 33.84 |
| HETATM | 12127 | O | HOH | | 91 | 46.666 | 25.435 | 39.522 | 1.00 | 46.43 |
| HETATM | 12128 | O | HOH | | 92 | 46.232 | 22.556 | 41.496 | 1.00 | 42.39 |
| HETATM | 12129 | O | HOH | | 93 | 59.548 | 45.139 | 35.000 | 1.00 | 42.25 |
| HETATM | 12130 | O | HOH | | 94 | 62.857 | 46.175 | 65.622 | 1.00 | 19.05 |
| HETATM | 12131 | O | HOH | | 95 | 55.251 | 51.600 | 69.221 | 1.00 | 24.07 |
| HETATM | 12132 | O | HOH | | 96 | 67.380 | 57.546 | 70.835 | 1.00 | 28.58 |
| HETATM | 12133 | O | HOH | | 97 | 70.645 | 58.099 | 73.586 | 1.00 | 30.64 |
| HETATM | 12134 | O | HOH | | 98 | 74.810 | 58.797 | 72.603 | 1.00 | 27.25 |
| HETATM | 12135 | O | HOH | | 99 | 77.511 | 55.043 | 76.924 | 1.00 | 26.31 |
| HETATM | 12136 | O | HOH | | 100 | 60.609 | 64.994 | 69.994 | 1.00 | 34.49 |
| HETATM | 12137 | O | HOH | | 101 | 61.574 | 66.028 | 75.139 | 1.00 | 31.31 |
| HETATM | 12138 | O | HOH | | 102 | 68.125 | 68.933 | 71.197 | 1.00 | 32.38 |
| HETATM | 12139 | O | HOH | | 103 | 93.361 | 75.876 | 61.155 | 1.00 | 50.17 |
| HETATM | 12140 | O | HOH | | 104 | 92.339 | 75.801 | 77.524 | 1.00 | 44.12 |
| HETATM | 12141 | O | HOH | | 105 | 89.509 | 76.668 | 77.238 | 1.00 | 36.60 |
| HETATM | 12142 | O | HOH | | 106 | 96.453 | 75.594 | 77.314 | 1.00 | 34.90 |
| HETATM | 12143 | O | HOH | | 107 | 83.581 | 63.013 | 51.557 | 1.00 | 28.63 |
| HETATM | 12144 | O | HOH | | 108 | 76.910 | 56.310 | 50.467 | 1.00 | 28.57 |
| HETATM | 12145 | O | HOH | | 109 | 88.046 | 54.771 | 55.131 | 1.00 | 44.11 |
| HETATM | 12146 | O | HOH | | 110 | 80.838 | 89.466 | 77.925 | 1.00 | 33.60 |
| HETATM | 12147 | O | HOH | | 111 | 81.869 | 77.174 | 92.013 | 1.00 | 26.28 |
| HETATM | 12148 | O | HOH | | 112 | 85.641 | 73.512 | 93.741 | 1.00 | 26.83 |
| HETATM | 12149 | O | HOH | | 113 | 88.338 | 80.641 | 90.398 | 1.00 | 40.73 |
| HETATM | 12150 | O | HOH | | 114 | 72.839 | 75.180 | 103.334 | 1.00 | 31.56 |
| HETATM | 12151 | O | HOH | | 115 | 76.390 | 64.819 | 106.832 | 1.00 | 32.74 |
| HETATM | 12152 | O | HOH | | 116 | 70.062 | 56.424 | 104.604 | 1.00 | 34.31 |
| HETATM | 12153 | O | HOH | | 117 | 67.748 | 54.004 | 101.069 | 1.00 | 38.13 |
| HETATM | 12154 | O | HOH | | 118 | 68.759 | 50.320 | 98.367 | 1.00 | 57.66 |
| HETATM | 12155 | O | HOH | | 119 | 84.379 | 38.809 | 82.154 | 1.00 | 36.63 |
| HETATM | 12156 | O | HOH | | 120 | 73.754 | 50.392 | 47.910 | 1.00 | 21.15 |
| HETATM | 12157 | O | HOH | | 121 | 78.379 | 46.740 | 34.934 | 1.00 | 24.31 |
| HETATM | 12158 | O | HOH | | 122 | 63.544 | 35.488 | 9.380 | 1.00 | 31.34 |
| HETATM | 12159 | O | HOH | | 123 | 44.179 | 34.979 | 13.105 | 1.00 | 25.61 |
| HETATM | 12160 | O | HOH | | 124 | 52.909 | 50.685 | 14.735 | 1.00 | 34.54 |
| HETATM | 12161 | O | HOH | | 125 | 50.961 | 44.497 | 18.295 | 1.00 | 23.10 |
| HETATM | 12162 | O | HOH | | 126 | 36.677 | 57.474 | 22.449 | 1.00 | 29.77 |
| HETATM | 12163 | O | HOH | | 127 | 41.507 | 53.667 | 24.386 | 1.00 | 51.04 |
| HETATM | 12164 | O | HOH | | 128 | 27.040 | 64.555 | 5.523 | 1.00 | 39.18 |
| HETATM | 12165 | O | HOH | | 129 | 20.081 | 69.745 | 25.535 | 1.00 | 36.99 |
| HETATM | 12166 | O | HOH | | 130 | 22.434 | 70.829 | 16.822 | 1.00 | 37.46 |
| HETATM | 12167 | O | HOH | | 131 | 41.884 | 83.942 | 26.261 | 1.00 | 44.34 |
| HETATM | 12168 | O | HOH | | 132 | 39.866 | 65.248 | 76.576 | 1.00 | 25.68 |
| HETATM | 12169 | O | HOH | | 133 | 36.727 | 68.844 | 86.164 | 1.00 | 37.09 |
| HETATM | 12170 | O | HOH | | 134 | 62.595 | 52.028 | 99.281 | 1.00 | 43.29 |
| HETATM | 12171 | O | HOH | | 135 | 63.099 | 64.910 | 92.518 | 1.00 | 26.46 |
| HETATM | 12172 | O | HOH | | 136 | 66.993 | 73.636 | 91.917 | 1.00 | 35.76 |
| HETATM | 12173 | O | HOH | | 137 | 63.825 | 76.390 | 90.847 | 1.00 | 40.25 |
| HETATM | 12174 | O | HOH | | 138 | 57.554 | 81.223 | 81.797 | 1.00 | 49.39 |
| HETATM | 12175 | O | HOH | | 139 | 51.439 | 80.508 | 87.594 | 1.00 | 31.00 |
| HETATM | 12176 | O | HOH | | 140 | 50.917 | 67.304 | 92.867 | 1.00 | 29.73 |
| HETATM | 12177 | O | HOH | | 141 | 53.638 | 67.099 | 74.968 | 1.00 | 27.88 |
| HETATM | 12178 | O | HOH | | 142 | 59.142 | 88.533 | 56.693 | 1.00 | 40.75 |
| HETATM | 12179 | O | HOH | | 143 | 58.554 | 79.052 | 50.505 | 1.00 | 29.32 |
| HETATM | 12180 | O | HOH | | 144 | 67.618 | 71.785 | 44.131 | 1.00 | 24.36 |
| HETATM | 12181 | O | HOH | | 145 | 53.907 | 81.654 | 47.344 | 1.00 | 43.67 |
| HETATM | 12182 | O | HOH | | 146 | 53.793 | 62.777 | −3.567 | 1.00 | 29.70 |
| HETATM | 12183 | O | HOH | | 147 | 64.234 | 48.230 | 24.925 | 1.00 | 33.58 |
| HETATM | 12184 | O | HOH | | 148 | 66.492 | 30.910 | 47.228 | 1.00 | 29.81 |
| HETATM | 12185 | O | HOH | | 202 | 32.619 | 40.712 | 51.879 | 1.00 | 30.37 |
| HETATM | 12186 | O | HOH | | 204 | 43.839 | 44.592 | 49.564 | 1.00 | 28.20 |
| HETATM | 12187 | O | HOH | | 206 | 37.580 | 57.461 | 52.650 | 1.00 | 27.92 |
| HETATM | 12188 | O | HOH | | 208 | 42.183 | 61.804 | 55.700 | 1.00 | 34.76 |
| HETATM | 12189 | O | HOH | | 210 | 39.538 | 58.414 | 54.332 | 1.00 | 25.41 |
| HETATM | 12190 | O | HOH | | 212 | 48.967 | 56.509 | 65.352 | 1.00 | 44.84 |
| HETATM | 12191 | O | HOH | | 214 | 71.887 | 58.634 | 75.646 | 1.00 | 28.47 |
| HETATM | 12192 | O | HOH | | 216 | 71.427 | 55.779 | 72.292 | 1.00 | 35.84 |
| HETATM | 12193 | O | HOH | | 218 | 70.822 | 58.005 | 77.949 | 1.00 | 30.76 |
| HETATM | 12194 | O | HOH | | 220 | 69.998 | 78.732 | 78.356 | 1.00 | 22.45 |
| HETATM | 12195 | O | HOH | | 222 | 71.248 | 86.759 | 79.600 | 1.00 | 38.95 |
| HETATM | 12196 | O | HOH | | 224 | 56.680 | 84.166 | 88.555 | 1.00 | 37.10 |
| HETATM | 12197 | O | HOH | | 226 | 57.373 | 80.916 | 86.390 | 1.00 | 49.30 |
| HETATM | 12198 | O | HOH | | 228 | 75.894 | 84.232 | 74.472 | 1.00 | 28.54 |
| HETATM | 12199 | O | HOH | | 230 | 75.429 | 94.083 | 66.254 | 1.00 | 48.76 |
| HETATM | 12200 | O | HOH | | 232 | 56.996 | 84.593 | 55.734 | 1.00 | 33.84 |
| HETATM | 12201 | O | HOH | | 234 | 64.723 | 80.578 | 51.996 | 1.00 | 38.72 |
| HETATM | 12202 | O | HOH | | 236 | 51.212 | 78.791 | 54.717 | 1.00 | 25.78 |
| HETATM | 12203 | O | HOH | | 238 | 48.051 | 75.518 | 66.773 | 1.00 | 29.08 |
| HETATM | 12204 | O | HOH | | 240 | 41.990 | 72.224 | 71.279 | 1.00 | 32.98 |
| HETATM | 12205 | O | HOH | | 242 | 39.086 | 89.203 | 74.974 | 1.00 | 26.27 |
| HETATM | 12206 | O | HOH | | 244 | 81.152 | 36.150 | 29.584 | 1.00 | 33.69 |
| HETATM | 12207 | O | HOH | | 245 | 78.600 | 49.867 | 34.219 | 1.00 | 26.08 |
| HETATM | 12208 | O | HOH | | 246 | 51.499 | 59.265 | −6.134 | 1.00 | 34.07 |
| HETATM | 12209 | O | HOH | | 247 | 46.560 | 55.997 | 30.696 | 1.00 | 26.99 |
| HETATM | 12210 | O | HOH | | 248 | 51.695 | 64.028 | 29.990 | 1.00 | 23.45 |
| HETATM | 12211 | O | HOH | | 249 | 50.152 | 59.677 | 36.121 | 1.00 | 27.45 |
| HETATM | 12212 | O | HOH | | 250 | 53.824 | 56.732 | 35.090 | 1.00 | 31.08 |
| HETATM | 12213 | O | HOH | | 251 | 52.542 | 60.702 | 34.717 | 1.00 | 33.68 |
| HETATM | 12214 | O | HOH | | 252 | 57.043 | 64.788 | 39.705 | 1.00 | 38.98 |
| HETATM | 12215 | O | HOH | | 253 | 54.472 | 63.858 | 40.439 | 1.00 | 29.74 |
| HETATM | 12216 | O | HOH | | 254 | 47.192 | 45.492 | 35.067 | 1.00 | 38.53 |
| HETATM | 12217 | O | HOH | | 255 | 42.136 | 51.046 | 33.979 | 1.00 | 37.48 |
| HETATM | 12218 | O | HOH | | 256 | 48.624 | 43.656 | 28.724 | 1.00 | 35.99 |
| HETATM | 12219 | O | HOH | | 257 | 53.099 | 40.645 | 24.972 | 1.00 | 24.40 |
| HETATM | 12220 | O | HOH | | 258 | 49.300 | 44.995 | 31.514 | 1.00 | 35.06 |
| HETATM | 12221 | O | HOH | | 259 | 46.593 | 49.097 | 33.004 | 1.00 | 45.88 |
| HETATM | 12222 | O | HOH | | 260 | 44.469 | 49.208 | 32.906 | 1.00 | 39.68 |
| HETATM | 12223 | O | HOH | | 261 | 41.442 | 46.842 | 26.549 | 1.00 | 26.59 |
| HETATM | 12224 | O | HOH | | 262 | 43.277 | 51.017 | 31.045 | 1.00 | 40.35 |
| HETATM | 12225 | O | HOH | | 263 | 39.106 | 54.477 | 24.710 | 1.00 | 57.23 |
| HETATM | 12226 | O | HOH | | 264 | 40.193 | 61.253 | 24.178 | 1.00 | 27.83 |
| HETATM | 12227 | O | HOH | | 265 | 43.286 | 61.838 | 21.847 | 1.00 | 47.16 |
| HETATM | 12228 | O | HOH | | 266 | 35.232 | 59.526 | 15.974 | 1.00 | 33.54 |
| HETATM | 12229 | O | HOH | | 267 | 38.799 | 65.129 | 17.277 | 1.00 | 32.63 |
| HETATM | 12230 | O | HOH | | 268 | 45.335 | 68.698 | 15.891 | 1.00 | 39.70 |
| HETATM | 12231 | O | HOH | | 269 | 47.617 | 70.146 | 23.139 | 1.00 | 31.12 |
| HETATM | 12232 | O | HOH | | 270 | 34.678 | 53.242 | 13.234 | 1.00 | 31.57 |
| HETATM | 12233 | O | HOH | | 271 | 29.153 | 73.980 | 30.341 | 1.00 | 43.04 |
| HETATM | 12234 | O | HOH | | 272 | 28.449 | 76.672 | 29.593 | 1.00 | 31.36 |
| HETATM | 12235 | O | HOH | | 273 | 34.907 | 79.293 | 33.911 | 1.00 | 38.34 |
| HETATM | 12236 | O | HOH | | 274 | 45.725 | 61.512 | 35.273 | 1.00 | 22.54 |
| HETATM | 12237 | O | HOH | | 275 | 39.889 | 61.856 | 39.172 | 1.00 | 22.94 |
| HETATM | 12238 | O | HOH | | 276 | 41.209 | 65.035 | 39.137 | 1.00 | 33.27 |
| HETATM | 12239 | O | HOH | | 277 | 27.132 | 67.569 | 7.800 | 1.00 | 44.95 |
| HETATM | 12240 | O | HOH | | 278 | 24.932 | 69.691 | 8.276 | 1.00 | 47.36 |
| HETATM | 12241 | O | HOH | | 279 | 35.912 | 64.257 | 50.788 | 1.00 | 53.43 |
| HETATM | 12242 | O | HOH | | 280 | 60.943 | 79.936 | 49.167 | 1.00 | 27.23 |
| HETATM | 12243 | O | HOH | | 281 | 65.094 | 67.150 | 70.063 | 1.00 | 29.89 |
| HETATM | 12244 | O | HOH | | 282 | 67.023 | 65.591 | 68.876 | 1.00 | 32.35 |
| HETATM | 12245 | O | HOH | | 283 | 64.065 | 65.176 | 70.832 | 1.00 | 43.75 |
| HETATM | 12246 | O | HOH | | 284 | 60.509 | 67.789 | 68.395 | 1.00 | 22.45 |
| HETATM | 12247 | O | HOH | | 285 | 57.398 | 66.633 | 70.380 | 1.00 | 33.83 |
| HETATM | 12248 | O | HOH | | 286 | 58.553 | 64.183 | 70.306 | 1.00 | 45.07 |
| HETATM | 12249 | O | HOH | | 287 | 28.754 | 79.787 | 24.414 | 1.00 | 41.13 |
| HETATM | 12250 | O | HOH | | 288 | 27.759 | 71.284 | 45.936 | 1.00 | 47.91 |
| HETATM | 12251 | O | HOH | | 289 | 23.927 | 72.799 | 35.757 | 1.00 | 51.30 |
| HETATM | 12252 | O | HOH | | 290 | 29.955 | 73.971 | 39.463 | 1.00 | 36.46 |
| HETATM | 12253 | O | HOH | | 291 | 25.897 | 53.293 | 41.801 | 1.00 | 33.14 |
| HETATM | 12254 | O | HOH | | 292 | 23.797 | 50.547 | 38.975 | 1.00 | 31.04 |
| HETATM | 12255 | O | HOH | | 293 | 26.779 | 49.888 | 39.145 | 1.00 | 36.09 |
| HETATM | 12256 | O | HOH | | 294 | 27.839 | 58.254 | 37.402 | 1.00 | 26.51 |
| HETATM | 12257 | O | HOH | | 295 | 29.803 | 58.215 | 43.171 | 1.00 | 23.11 |
| HETATM | 12258 | O | HOH | | 296 | 29.469 | 60.011 | 41.576 | 1.00 | 34.44 |
| HETATM | 12259 | O | HOH | | 297 | 32.193 | 40.552 | 38.804 | 1.00 | 45.78 |
| HETATM | 12260 | O | HOH | | 298 | 33.709 | 34.220 | 29.537 | 1.00 | 32.56 |
| HETATM | 12261 | O | HOH | | 299 | 39.324 | 47.614 | 21.483 | 1.00 | 33.19 |
| HETATM | 12262 | O | HOH | | 300 | 33.791 | 44.525 | 25.455 | 1.00 | 34.40 |
| HETATM | 12263 | O | HOH | | 301 | 34.210 | 32.867 | 17.969 | 1.00 | 23.95 |
| HETATM | 12264 | O | HOH | | 302 | 23.518 | 42.390 | 14.824 | 1.00 | 33.39 |
| HETATM | 12265 | O | HOH | | 303 | 28.153 | 45.492 | 6.361 | 1.00 | 30.26 |
| HETATM | 12266 | O | HOH | | 304 | 26.608 | 48.522 | 7.079 | 1.00 | 29.68 |
| HETATM | 12267 | O | HOH | | 305 | 38.605 | 48.045 | −0.774 | 1.00 | 48.54 |
| HETATM | 12268 | O | HOH | | 306 | 36.442 | 48.639 | −1.382 | 1.00 | 51.66 |
| HETATM | 12269 | O | HOH | | 307 | 33.276 | 49.992 | 5.200 | 1.00 | 34.73 |
| HETATM | 12270 | O | HOH | | 308 | 34.560 | 28.406 | −1.463 | 1.00 | 56.41 |
| HETATM | 12271 | O | HOH | | 309 | 46.509 | 52.025 | 11.464 | 1.00 | 23.72 |
| HETATM | 12272 | O | HOH | | 310 | 40.013 | 51.475 | 8.495 | 1.00 | 39.95 |
| HETATM | 12273 | O | HOH | | 311 | 63.562 | 52.804 | 2.547 | 1.00 | 38.56 |
| HETATM | 12274 | O | HOH | | 312 | 66.967 | 44.809 | 5.191 | 1.00 | 43.64 |
| HETATM | 12275 | O | HOH | | 313 | 76.726 | 33.117 | 24.145 | 1.00 | 31.10 |
| HETATM | 12276 | O | HOH | | 314 | 45.201 | 27.566 | 28.129 | 1.00 | 32.65 |
| HETATM | 12277 | O | HOH | | 315 | 62.406 | 37.653 | 31.681 | 1.00 | 33.49 |
| HETATM | 12278 | O | HOH | | 316 | 67.033 | 50.301 | 26.622 | 1.00 | 28.28 |
| HETATM | 12279 | O | HOH | | 317 | 48.216 | 37.093 | 36.293 | 1.00 | 27.89 |
| HETATM | 12280 | O | HOH | | 318 | 36.680 | 27.536 | 26.666 | 1.00 | 43.58 |
| HETATM | 12281 | O | HOH | | 319 | 42.690 | 28.000 | 29.436 | 1.00 | 29.76 |
| HETATM | 12282 | O | HOH | | 320 | 47.256 | 39.106 | 52.493 | 1.00 | 27.93 |
| HETATM | 12283 | O | HOH | | 321 | 58.126 | 34.638 | 53.518 | 1.00 | 32.97 |
| HETATM | 12284 | O | HOH | | 322 | 64.011 | 42.183 | 54.777 | 1.00 | 26.52 |
| HETATM | 12285 | O | HOH | | 323 | 57.427 | 64.632 | 46.535 | 1.00 | 24.28 |
| HETATM | 12286 | O | HOH | | 324 | 56.723 | 63.053 | 51.391 | 1.00 | 23.85 |
| HETATM | 12287 | O | HOH | | 325 | 67.474 | 64.172 | 35.795 | 1.00 | 26.71 |
| HETATM | 12288 | O | HOH | | 326 | 65.117 | 63.674 | 33.106 | 1.00 | 34.74 |
| HETATM | 12289 | O | HOH | | 327 | 77.532 | 52.988 | 43.002 | 1.00 | 35.17 |
| HETATM | 12290 | O | HOH | | 328 | 73.665 | 41.787 | 70.523 | 1.00 | 23.80 |
| HETATM | 12291 | O | HOH | | 329 | 74.243 | 39.155 | 71.502 | 1.00 | 37.50 |
| HETATM | 12292 | O | HOH | | 330 | 65.915 | 51.647 | 74.886 | 1.00 | 33.80 |
| HETATM | 12293 | O | HOH | | 331 | 63.198 | 51.539 | 76.002 | 1.00 | 44.63 |
| HETATM | 12294 | O | HOH | | 332 | 68.579 | 56.719 | 74.627 | 1.00 | 36.47 |
| HETATM | 12295 | O | HOH | | 333 | 62.332 | 54.612 | 89.660 | 1.00 | 36.15 |
| HETATM | 12296 | O | HOH | | 334 | 59.454 | 68.706 | 111.542 | 1.00 | 31.80 |
| HETATM | 12297 | O | HOH | | 335 | 53.783 | 65.446 | 77.107 | 1.00 | 35.33 |
| HETATM | 12298 | O | HOH | | 336 | 52.096 | 74.528 | 87.111 | 1.00 | 54.53 |
| HETATM | 12299 | O | HOH | | 337 | 53.792 | 79.518 | 82.367 | 1.00 | 42.24 |
| HETATM | 12300 | O | HOH | | 338 | 45.757 | 92.494 | 97.309 | 1.00 | 39.29 |
| HETATM | 12301 | O | HOH | | 339 | 39.105 | 56.189 | 55.767 | 1.00 | 27.64 |
| HETATM | 12302 | O | HOH | | 340 | 43.199 | 92.659 | 61.430 | 1.00 | 37.84 |
| HETATM | 12303 | O | HOH | | 341 | 53.836 | 85.197 | 55.803 | 1.00 | 33.80 |
| HETATM | 12304 | O | HOH | | 342 | 53.706 | 94.980 | 73.302 | 1.00 | 28.63 |
| HETATM | 12305 | O | HOH | | 343 | 51.760 | 94.004 | 75.045 | 1.00 | 33.39 |
| HETATM | 12306 | O | HOH | | 344 | 58.030 | 89.168 | 61.516 | 1.00 | 25.90 |
| HETATM | 12307 | O | HOH | | 345 | 50.970 | 107.755 | 84.519 | 1.00 | 48.79 |
| HETATM | 12308 | O | HOH | | 346 | 64.514 | 83.981 | 93.646 | 1.00 | 31.88 |
| HETATM | 12309 | O | HOH | | 347 | 80.236 | 91.940 | 81.786 | 1.00 | 40.68 |
| HETATM | 12310 | O | HOH | | 348 | 75.328 | 85.599 | 104.775 | 1.00 | 24.56 |
| HETATM | 12311 | O | HOH | | 349 | 79.517 | 79.180 | 102.402 | 1.00 | 25.88 |
| HETATM | 12312 | O | HOH | | 350 | 80.747 | 63.743 | 95.369 | 1.00 | 27.89 |
| HETATM | 12313 | O | HOH | | 351 | 64.969 | 77.943 | 106.765 | 1.00 | 44.27 |
| HETATM | 12314 | O | HOH | | 352 | 95.965 | 80.561 | 67.682 | 1.00 | 44.80 |
| HETATM | 12315 | O | HOH | | 353 | 86.914 | 49.199 | 79.546 | 1.00 | 28.91 |
| HETATM | 12316 | O | HOH | | 354 | 77.363 | 57.263 | 75.679 | 1.00 | 24.96 |
| HETATM | 12317 | O | HOH | | 355 | 78.207 | 53.138 | 78.606 | 1.00 | 31.72 |
| HETATM | 12318 | O | HOH | | 356 | 87.975 | 68.747 | 54.296 | 1.00 | 50.80 |
| HETATM | 12319 | O | HOH | | 357 | 85.047 | 62.868 | 55.786 | 1.00 | 31.85 |
| HETATM | 12320 | O | HOH | | 358 | 86.034 | 61.805 | 52.552 | 1.00 | 31.60 |
| HETATM | 12321 | O | HOH | | 359 | 79.445 | 74.128 | 45.275 | 1.00 | 33.57 |
| HETATM | 12322 | O | HOH | | 360 | 56.053 | 54.524 | 102.588 | 1.00 | 171.18 |
| HETATM | 12323 | O | HOH | | 361 | 48.029 | 63.170 | 110.923 | 1.00 | 47.18 |
| HETATM | 12324 | O | HOH | | 362 | 51.605 | 65.693 | 106.423 | 1.00 | 47.01 |
| HETATM | 12325 | O | HOH | | 363 | 50.673 | 68.039 | 105.495 | 1.00 | 46.13 |
| HETATM | 12326 | O | HOH | | 364 | 94.322 | 44.608 | 67.120 | 1.00 | 45.40 |
| HETATM | 12327 | O | HOH | | 365 | 86.923 | 43.646 | 74.686 | 1.00 | 33.81 |
| HETATM | 12328 | O | HOH | | 366 | 79.642 | 38.900 | 69.678 | 1.00 | 50.27 |
| HETATM | 12329 | O | HOH | | 367 | 67.633 | 24.490 | 28.602 | 1.00 | 52.13 |
| HETATM | 12330 | O | HOH | | 368 | 54.251 | 58.966 | 34.469 | 1.00 | 43.40 |
| HETATM | 12331 | O | HOH | | 369 | 51.371 | 57.464 | 36.619 | 1.00 | 24.00 |
| HETATM | 12332 | O | HOH | | 370 | 59.016 | 48.135 | 34.799 | 1.00 | 40.23 |
| HETATM | 12333 | O | HOH | | 371 | 34.879 | 31.553 | 9.868 | 1.00 | 44.94 |
| HETATM | 12334 | O | HOH | | 372 | 27.580 | 41.566 | 39.476 | 1.00 | 42.65 |
| HETATM | 12335 | O | HOH | | 373 | 24.846 | 42.734 | 35.135 | 1.00 | 51.21 |
| HETATM | 12336 | O | HOH | | 374 | 19.556 | 46.158 | 34.315 | 1.00 | 53.49 |
| HETATM | 12337 | O | HOH | | 375 | 83.691 | 70.175 | 77.027 | 1.00 | 28.28 |
| HETATM | 12338 | O | HOH | | 376 | 74.717 | 68.866 | 78.833 | 1.00 | 37.76 |
| HETATM | 12339 | O | HOH | | 377 | 76.631 | 68.088 | 80.362 | 1.00 | 29.47 |
| HETATM | 12340 | O | HOH | | 378 | 58.860 | 55.128 | 1.822 | 1.00 | 30.66 |
| HETATM | 12341 | O | HOH | | 379 | 62.809 | 55.151 | −3.277 | 1.00 | 40.41 |
| HETATM | 12342 | O | HOH | | 380 | 33.273 | 62.466 | 49.352 | 1.00 | 41.97 |
| HETATM | 12343 | O | HOH | | 381 | 28.588 | 59.352 | 49.926 | 1.00 | 41.32 |
| HETATM | 12344 | O | HOH | | 382 | 30.906 | 56.703 | 47.696 | 1.00 | 41.49 |
| HETATM | 12345 | O | HOH | | 383 | 35.506 | 55.437 | 50.284 | 1.00 | 36.64 |
| HETATM | 12346 | O | HOH | | 384 | 87.842 | 80.426 | 66.401 | 1.00 | 43.96 |
| HETATM | 12347 | O | HOH | | 385 | 86.490 | 79.913 | 76.221 | 1.00 | 32.49 |
| HETATM | 12348 | O | HOH | | 386 | 84.867 | 74.141 | 57.146 | 1.00 | 40.97 |
| HETATM | 12349 | O | HOH | | 387 | 82.643 | 79.545 | 52.006 | 1.00 | 50.83 |
| HETATM | 12350 | O | HOH | | 388 | 68.042 | 83.874 | 47.140 | 1.00 | 49.08 |
| HETATM | 12351 | O | HOH | | 389 | 52.056 | 92.832 | 68.966 | 1.00 | 43.35 |
| HETATM | 12352 | O | HOH | | 390 | 54.797 | 93.224 | 71.916 | 1.00 | 40.36 |
| HETATM | 12353 | O | HOH | | 391 | 57.293 | 91.228 | 67.268 | 1.00 | 24.55 |
| HETATM | 12354 | O | HOH | | 392 | 56.898 | 89.074 | 65.525 | 1.00 | 32.59 |
| HETATM | 12355 | O | HOH | | 393 | 55.335 | 90.968 | 68.860 | 1.00 | 25.76 |
| HETATM | 12356 | O | HOH | | 394 | 56.153 | 89.417 | 62.819 | 1.00 | 30.99 |
| HETATM | 12357 | O | HOH | | 395 | 59.579 | 102.134 | 76.705 | 1.00 | 40.70 |
| HETATM | 12358 | O | HOH | | 396 | 61.841 | 100.995 | 93.173 | 1.00 | 50.46 |
| HETATM | 12359 | O | HOH | | 397 | 71.154 | 98.292 | 81.895 | 1.00 | 31.13 |
| HETATM | 12360 | O | HOH | | 398 | 75.477 | 93.747 | 78.061 | 1.00 | 36.92 |
| HETATM | 12361 | O | HOH | | 399 | 79.703 | 89.990 | 74.196 | 1.00 | 49.92 |
| HETATM | 12362 | O | HOH | | 400 | 85.642 | 70.504 | 75.265 | 1.00 | 34.21 |
| HETATM | 12363 | HG | HG | Y | 303 | 42.410 | 43.821 | 32.702 | 1.00 | 59.73 |
| HETATM | 12364 | HG | HG | Y | 301 | 35.399 | 52.819 | 33.178 | 1.00 | 65.74 |
| HETATM | 12365 | HG | HG | Y | 302 | 36.321 | 52.198 | 31.093 | 1.00 | 103.48 |
| HETATM | 12366 | HG | HG | Z | 303 | 73.145 | 77.979 | 72.298 | 1.00 | 63.81 |
| HETATM | 12367 | HG | HG | Z | 301 | 63.582 | 84.279 | 71.535 | 1.00 | 65.41 |
| HETATM | 12368 | HG | HG | Z | 302 | 64.171 | 83.832 | 74.081 | 1.00 | 106.14 |
| END | | | | | | | | | | |

Column 2 lists A number for the atom in the structure.
Column 3 lists the element whose coordinates are measured. The first letter in the column defines the element.
Column 4 lists the type of amino acid.
Column 5 lists A number for the amino acid in the structure.
Columns 6-8 list the crystallographic coordinates X, Y, and Z respectively. The crystallographic coordinates define the atomic position of the element measured.
Column 9 lists an occupancy factor that refers to the fraction of the molecules in which each atom occupies the position specified by the coordinates. A value of “1” indicates that each atom has the same conformation, i.e., the same position, in all molecules of the crystal.
Column 10 lists a thermal factor “B” that measures movement of the atom around its atomic center.

What is claimed is:

1. A crystal of the extracellular domain of mammalian DPP-IV, wherein the crystal has an orthorhombic space group of P2₁2₁2₁ and one homodimer of DPP-IV in the asymmetric unit.

2. The crystal of claim 1, wherein the crystal has unit cell dimensions of:
a is from 63 Å to 70 Å;
b is from 66 Å to 70 Å;
c is from 416 Å to 424 Å;
and a P2₁2₁2₁ symmetry.

3. The crystal of claim 2, wherein the crystal has the atomic structure coordinates according to Table 4.

4. A co-crystal of the extracellular domain of mammalian DPP-IV which comprises a ligand bound to the active site of the mammalion DPP-IV, wherein the crystal has an orthorhombic space group of P2₁2₁2₁ and one homodimer of DPP-IV in the asymmetric unit.

5. The co-crystal of claim 4, wherein the co-crystal has unit cell dimensions of:
a is from 63 Å to 70 Å;
b is from 66 Å to 70 Å;
c is from 416 Å to 424 Å;
and a P2₁2₁2₁ symmetry.

6. The co-crystal of claim 4 further comprising HgCl₂.

7. A co-crystal of the extracellular domain of mammalian DPP-IV which comprises a ligand bound to an allosteric binding site of the mammalian DPP-IV, wherein the crystal has an orthorhombic space group of P2₁2₁2₁ and one homodimer of DPP-IV in the asymmetric unit.

8. The co-crystal of claim 7 further comprising HgCl₂.

9. A method for crystallizing mammalian DPP-IV, the method comprising
(a) providing a buffered, aqueous solution of pH 7 to 8.5 with a concentration of 7 mg/ml to 22 mg/ml of the extracellular domain of mammalian DPP-IV; and
(b) growing crystals by vapor diffusion using a buffered reservoir solution with between 10% and 30% PEG, between 10% and 20% glycerol, wherein PEG has an average molecular weight between 1000 and 20000.

10. The method according to claim 9, wherein the extracellular domain of mammalian DPP-IV of step (a) is produced in P. pastoris and then deglycosylated.

11. A method for co-crystallizing mammalian DPP-IV and an active site ligand, the method comprising
(a) providing a buffered, aqueous solution of pH 7 to 8.5 with a concentration of 7 mg/ml to 22 mg/ml of the extracellular domain of mammalian DPP-IV;
(b) adding a molar excess of the active site ligand to the aqueous solution of mammalian DPP-IV;
(c) growing crystals by vapor diffusion using a buffered reservoir solution with between 10% and 30% PEG, between 10% and 20% glycerol, wherein PEG has an average molecular weight between 1000 and 20000.

12. The method according to claim 11, wherein the extracellular domain of mammalian DPP-IV of step (a) is produced in P. pastoris and then deglycosylated.

13. A crystal produced by the method according to claim 9.

14. A co-crystal produced by the method according to claim 11.

15. An isolated nucleic acid sequence which encodes the soluble extracellular domain of DPP-IV, comprising the nucleotide sequence of SEQ ID NO:1.

16. A nucleic acid construct comprising an expression vector and the nucleic acid sequence according to claim 15.

17. A host cell transformed with the nucleic acid construct according to claim 16.

18. A method of producing the soluble extracellular domain of DPP-IV comprising culturing the host cell of claim 17 under conditions permitting the expression of the soluble extracellular domain of DPP-IV by the host cell.

19. The method according to claim 18, wherein the host cell is P. pastoris.

20. A polypeptide comprising the soluble extracellular domain of DPP-IV as set forth in SEQ ID NO:2.


SEQUENCE LISTING (11 sequences)

SEQ ID NO: 1 - DNA, 2211 bp, Homo sapiens
ggcacagatgatgctacagctgacagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctggtcatatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttag

SEQ ID NO: 2 - protein, 736 aa, Homo sapiens
GTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 3 - protein, 765 aa, Homo sapiens
MTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 4 - protein, 767 aa, Rattus norvegicus
MKTPWKVLLGLLGAAALVTIITVPVVLLNKDEAAADSARTYTLADYLKNTFRVKSYSLRWVSDSEYLYKQENNILLFNAEHGNSSIFLENSTFEIFGDSISDYSVSPDRLFVLLEYNYVKQWRHSYTASYSIYDLNKRQLITEEKIPNNTQWITWSQEGHKLAYVWKNDIYVKIEPHLPSHRITSTGKENVIFNGINDWVYEEEIFGAYSALWWSPNGTFLAYAQFNDTGVPLIEYSFYSDESLQYPKTVWIPYPKAGAVNPTVKFFVVNTDSLSSTTTTIPMQITAPASVTTGDHYLCDVAWVSEDRISLQWLRRIQNYSVMAICDYDKTTLVWNCPTTQEHIETSATGWCGRFRPAEPHFTSDGSSFYKIVSDKDGYKHICQFQKDRKPEQVCTFITKGAWEVISIEALTSDYLYYISNEYKEMPGGRNLYKIQLTDHTNKKCLSCDLNPERCQYYSVSLSKEAKYYQLGCRGPGLPLYTLHRSTDQKELRVLEDNSALDKMLQDVQMPSKKLDFIVLNETRFWYQMILPPHFDKSKKYPLLIDVYAGPCSQKADAAFRLNWATYLASTENIIVASFDGRGSGYQGDKIMHAINKRLGTLEVEDQIEAARQFLKMGFVDSKRVAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDAGVDFQAMWYTDEDHGIASSTAHQHIYSHMSHFLQQCFSLR

SEQ ID NO: 5 - protein, 283 aa, Sus scrofa
KGIDASDYQTVQIFYPSKDGTKIPMFIVHKKGIKLDGSHPAFLYGYGGFNISITPNYSVSRLIFVRHMGGVLAVANIRGGGEYGETWHKGGILANKQNCFDDFQCAAEYLIKEGYTSPKRLTINGGSNGGLLVATCANQRPDLFGCVIAQVGVMDMLKFHKYTIGHAWTTDYGCSDSKQHFEWLIKYSPLHNVKLPEADDIQYPSMLLLTADHDDRVVPLHSLKFIATLQYIVGRSRKQNNPLLIHVDTKAGHGAGKPTAKVIEEVSDMFAFIARCLNIDWIP

SEQ ID NO: 6 - DNA, 28 bp, Artificial Sequence
  Description of Artificial Sequence Primer
tgctggaattcggcacagatgatgctac

SEQ ID NO: 7 - DNA, 23 bp, Artificial Sequence
  Description of Artificial Sequence Primer
gcatggtaccttgaggtgctaag

SEQ ID NO: 8 - protein, 5 aa, Unknown Organism
  Description of Unknown Organism Mammalian DPP-IV peptide fragment
  MOD_RES (2): Variable amino acid
GXSYG

SEQ ID NO: 9 - protein, 8 aa, Unknown Organism
  Description of Unknown Organism Mammalian conserved DPP-IV peptide fragment
  MOD_RES (3): Variable amino acid
  MOD_RES (8): Variable amino acid
DWXYEEEX

SEQ ID NO: 10 - protein, 4 aa, Homo sapiens
LVAR

SEQ ID NO: 11 - protein, 5 aa, Homo sapiens
ESSGR